(19) United States
(12) Patent Application Publication
(10) Pub. No.: US 2024/0349605 A1
(43) Pub. Date: Oct. 17, 2024

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR); Da Jung LEE, Daejeon (KR)

(21) Appl. No.: 18/293,874

(22) PCT Filed: Aug. 5, 2022
(86) PCT No.: PCT/KR2022/011706

§ 371 Date: Jan. 31, 2024

(30) Foreign Application Priority Data
Aug. 5, 2021 (KR) 10-2021-0103336

(51) Int. Cl. H10K 85/60; C07C 211/54; C07C 211/61; C07D 209/86; C07D 307/91; C07D 333/76; C07D 405/04; C07D 405/10; C07D 405/12; C07D 405/14; C07D 409/12; C07D 409/14; C09K 11/06; H10K 50/11; H10K 101/00

(52) U.S. Cl. CPC H10K 85/6574; C07C 211/54; C07C 211/61; C07D 209/86; C07D 307/91; C07D 333/76; C07D 405/04; C07D 405/10; C07D 405/12; C07D 405/14; C07D 409/12; C07D 409/14; C09K 11/06; H10K 85/622; H10K 85/626; H10K 85/633; H10K 85/636; H10K 85/654; H10K 85/6576; C07C 2603/18; C07C 2603/26; C07C 2603/42; C09K 2211/1003; C09K 2211/1018; H10K 50/11; H10K 85/6572; H10K 2101/90

(57) ABSTRACT

An organic light-emitting device. light emitting device having a light emitting layer including a compound of Chemical Formula 1 and a compound of Chemical Formula 2:

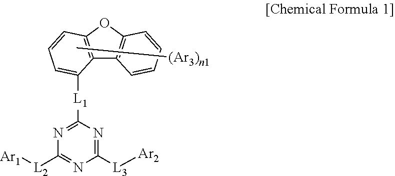

Ar₁ and Ar₂ are each independently a substituted or unsubstituted C₆₋₆₀ aryl or C₂₋₆₀ heteroaryl containing at least one of N, O and S, each Ar₃ is independently hydrogen, deuterium, or a substituted or unsubstituted C₆₋₆₀ aryl or C₂₋₆₀ heteroaryl containing at least one of N, O and S, and at least one of Ar₁, Ar₂-Ar₃ is a substituted or unsubstituted C₁₆₋₆₀ aryl polycyclic aromatic ring,

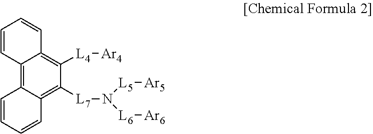

Ar₅ and Ar₆ are each substituted or unsubstituted C₆₋₆₀ aryl or C₂₋₆₀ heteroaryl containing at least one of N, O and S, and the other substituents are as defined in the specification, provided that Ar₃ is not

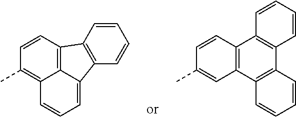

when Ar₃ is an unsubstituted C₁₆₋₆₀ aryl polycyclic aromatic ring.

### CROSS CITATION TO RELATED APPLICATION(S)

This application is a National Stage Application of International Application No. PCT/KR2022/011706 filed on Aug. 5, 2022, which claims the benefit of Korean Patent Application No. 10-2021-0103336 filed on Aug. 5, 2021 in the Korean Intellectual Property Office, the disclosures of which are incorporated herein by reference in their entirety.

### TECHNICAL FIELD

The present disclosure relates to an organic light emitting device.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of an organic light emitting device having improved driving voltage, efficiency, and lifespan.

### PRIOR ART LITERATURE

### Patent Literature

(Patent Literature 1) Korean Unexamined Patent Publication No. 10-2000-0051826

### BRIEF DESCRIPTION

### Technical Problem

The present disclosure relates to an organic light emitting device having improved driving voltage, efficiency, and lifespan.

### Technical Solution

In the present disclosure, provided is an organic light emitting device including
an anode;
a cathode; and
a light emitting layer that is provided between the anode and the cathode,
wherein the light emitting layer includes a compound of the following Chemical Formula 1 and a compound of the following Chemical Formula 2:

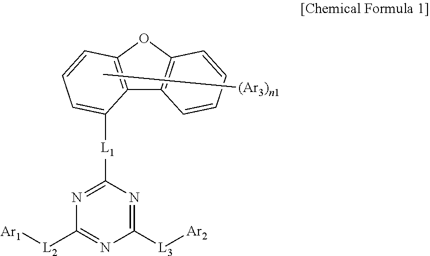

wherein in Chemical Formula 1:
L₁ to L₃ are each independently a single bond or a substituted or unsubstituted C₆₋₆₀ arylene;
Ar₁ and Ar₂ are each independently a substituted or unsubstituted C₆₋₆₀ aryl or a substituted or unsubstituted C₂₋₆₀ heteroaryl containing at least one heteroatom selected from the group consisting of N, O and S;
each Ar₃ is independently hydrogen, deuterium, a substituted or unsubstituted C₆₋₆₀ aryl, or a substituted or unsubstituted C₂₋₆₀ heteroaryl containing at least one heteroatom selected from the group consisting of N, O and S,
provided that at least one of Ar₁, Ar₂, and Ar₃ is substituted or unsubstituted C₁₆₋₆₀ aryl polycyclic aromatic ring; and
n1 is an integer of 0 to 7;

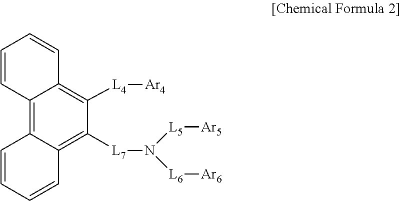

wherein in Chemical Formula 2:
Ar₄ is hydrogen, a substituted or unsubstituted C₆₋₆₀ aryl, or a substituted or unsubstituted C₂₋₆₀ heteroaryl containing at least one heteroatom selected from the group consisting of N, O and S;
Ar₅ and Ar₆ are each a substituted or unsubstituted C₆₋₆₀ aryl, or a substituted or unsubstituted C₂₋₆₀ heteroaryl containing at least one heteroatom selected from the group consisting of N, O and S;
L₄ to L₆ are each independently a single bond, a substituted or unsubstituted C₆₋₆₀ arylene, or a substituted or unsubstituted C₂₋₆₀ heteroarylene containing at least one heteroatom selected from the group consisting of N, O and S; and
L₇ is substituted or unsubstituted C₆₋₆₀ arylene,
provided that Ar₃ is not

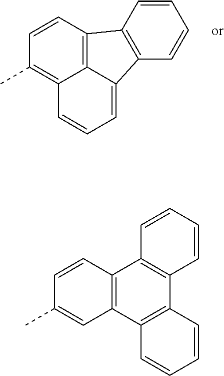

when Ar₃ is a substituted or unsubstituted C₁₆₋₆₀ aryl polycyclic aromatic ring.

### Advantageous Effects

The above-described organic light emitting device includes the compound of Chemical Formula 1 and the compound of Chemical Formula 2 in the light emitting layer, and thus can have improved efficiency, low driving voltage, and/or improved lifespan.

### BRIEF DESCRIPTION OF THE DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION OF THE EMBODIMENTS

Hereinafter, embodiments of the present disclosure will be described in more detail to facilitate understanding of the invention.

As used herein, the notation

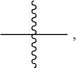

or [symbol] means a bond linked to another substituent group.

As used herein, the term “substituted or unsubstituted” means being unsubstituted or substituted with one or more substituents selected from the group consisting of deuterium; a halogen group; a nitrile group; a nitro group; a hydroxyl group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent in which two or more substituents of the above-exemplified substituents are connected to each other. For example, “a substituent in which two or more substituents are connected” can be a biphenyl group. Namely, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a substituent having the following structural formulae, but is not limited thereto:

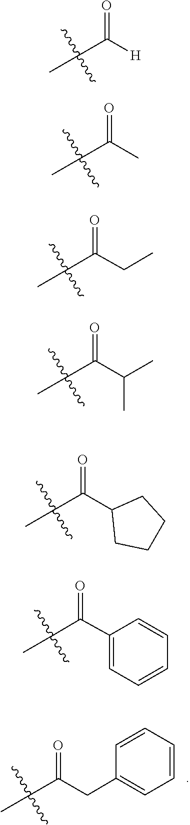

In the present disclosure, an ester group can have a structure in which oxygen of the ester group is substituted by a straight-chain, branched-chain, or cyclic alkyl group having 1 to 25 carbon atoms, or an aryl group having 6 to 25 carbon atoms. Specifically, the ester group can be a substituent having the following structural formulae, but is not limited thereto:

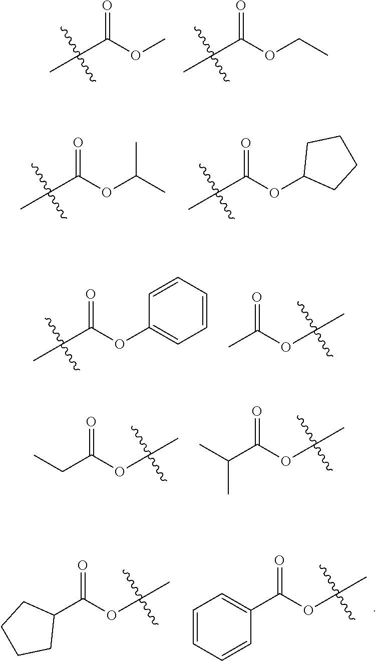

In the present disclosure, the carbon number of an imide group is not particularly limited, but is preferably 1 to 25. Specifically, the imide group can be a substituent having the following structural formulae, but is not limited thereto:

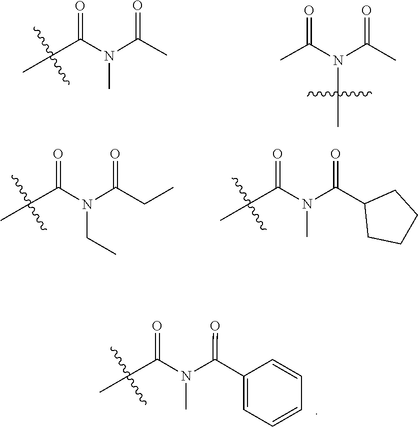

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, a phenylboron group and the like, but is not limited thereto.

In the present disclosure, examples of a halogen group include fluorine, chlorine, bromine, or iodine.

In the present disclosure, the alkyl group can be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl) vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group or the like, but is not limited thereto.

In the present disclosure, a fluorenyl group can be substituted, and two substituents can be bonded to each other to form a spiro structure. In the case where the fluorenyl group is substituted,

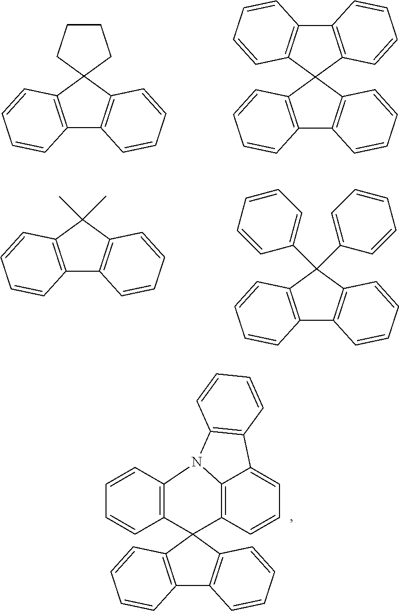

and the like can be formed. However, the structure is not limited thereto.

In the present disclosure, a heterocyclic group is a heterocyclic group containing at least one heteroatom of O, N, Si and S as a heterogeneous element, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

The present disclosure will be described in detail for each configuration.

### Anode and Cathode

The anode and cathode used in the present disclosure refer to electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Hole Injection Layer

The organic light emitting device according to the present disclosure can further include a hole injection layer on the anode, if necessary.

The hole injection layer is a layer for injecting holes from the electrode, and the hole injection material is preferably a compound which can transport the holes, thus has a hole-injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer.

Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

### Hole Transport Layer

The organic light emitting device according to the present disclosure can include a hole transport layer on the anode (or on the hole injection layer if there is a hole injection layer), if necessary.

The hole transport layer is a layer that receives holes from an anode or a hole injection layer and transports the holes to the light emitting layer. The hole transport material is suitably a material having large mobility to the holes, which can receive holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

### Electron Blocking Layer

The electron blocking layer is a layer provided between the hole transport layer and the light emitting layer in order to prevent electrons injected in the cathode from being transferred to the hole transport layer without being recombined in the light emitting layer, which can also be referred to as an electron inhibition layer or an electron stopping layer. The electron blocking layer is preferably a material having a smaller electron affinity than the electron transport layer.

### Light Emitting Layer

The organic light emitting device according to the present disclosure includes a light emitting layer between an anode and a cathode. The light emitting layer includes a compound of Chemical Formula 1 (hereinafter, ‘first compound’) and a compound of Chemical Formula 2 (hereinafter, ‘second compound’) as host materials. The light emitting layer used in the present disclosure means a layer that can emit light in the visible light region by combining holes and electrons transported from the anode and the cathode. Generally, the light emitting layer includes a host material and a dopant material, and in the present disclosure, the compound of Chemical Formula 1 and the compound of Chemical Formula 2 are included as a host. Specifically, the first compound functions as an N-type host material having an electron transport ability superior to a hole transport ability, and the second compound functions as a P-type host material having a hole transport ability superior to an electron transport ability, thereby maintaining the ratio of holes to electrons in the light emitting layer. Accordingly, the excitons emit lights evenly throughout the light emitting layer, so that the luminous efficiency and lifespan of the organic light emitting device can be simultaneously improved.

Hereinafter, the first compound and the second compound will be described.

### (First Compound)

The first compound is the following Chemical Formula 1. Specifically, it has a structure in which a triazinyl group is bonded to the 4-position of the dibenzofuran-based core through linker L₁ and at least one of Ar₁, Ar₂, and Ar₃, which are aryl or heteroaryl substituted to the dibenzofuran-based core or the triazinyl group, is a substituted or unsubstituted C₁₆₋₆₀ aryl polycyclic aromatic ring. In particular, the first compound according to the present disclosure has excellent electron transport capability, compared to a compound having a different structures or substituents, that is, a compound in which a naphthyl group or a phenanthryl group is substituted on the dibenzofuran-based core or the triazinyl group, or a compound in which the triazinyl group is substituted at a position other than the 4-position of the dibenzofuran-based core. Accordingly, the first compound according to the present disclosure has the specific structure and substituents as described above to increase the probability of recombination of holes and electrons in the light emitting layer by efficiently delivering electrons to a dopant material.

In Chemical Formula 1 related to the first compound included in the organic light-emitting device of the present disclosure,
L₁ to L₃ are each independently a single bond or substituted or unsubstituted C₆₋₆₀ arylene,
Ar₁ and Ar₂ are each independently a substituted or unsubstituted C₆₋₆₀ aryl, or substituted or unsubstituted C₂₋₆₀ heteroaryl containing at least one heteroatom selected from the group consisting of N, O and S,
each Ar₃ is independently hydrogen, deuterium, a substituted or unsubstituted C₆₋₆₀ aryl, or a substituted or unsubstituted C₂₋₆₀ heteroaryl containing at least one heteroatom selected from the group consisting of N, O and S,
provided that at least one of Ar₁, Ar₂, and Ar₃ is a substituted or unsubstituted C₁₆₋₆₀ aryl polycyclic aromatic ring, and
n1 is an integer of 0 to 7,
provided that Ar₃ is not

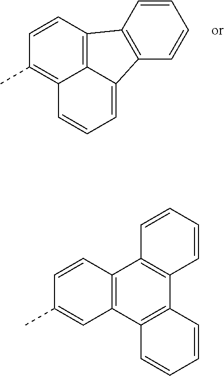

when Ar₃ is substituted or unsubstituted C₁₆₋₆₀ aryl polycyclic aromatic ring.

Specifically, the compound of Chemical Formula 1 is the following Chemical Formula 1-1 or Chemical Formula 1-2:

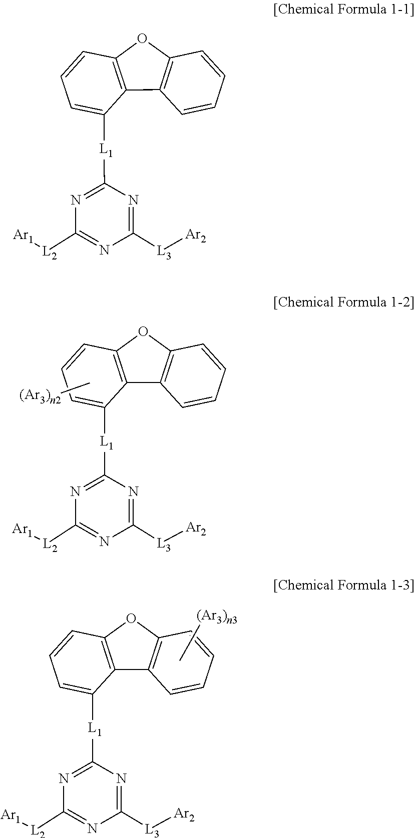

wherein in Chemical Formulae 1-1 to 1-3:
L₁ to L₃ and Ar₁ to Ar₃ are as defined in Chemical Formula 1,
n2 is an integer of 1 to 3, and
n3 is an integer of 1 to 4.

Preferably, in Chemical Formula 1 and Chemical Formulae 1-1 to 1-3, L₁ to L₃ can each independently be a single bond or a substituted or unsubstituted C₆₋₂₀ arylene.

Specifically, L₁ to L₃ can each independently be a single bond, phenylene, biphenylene, or naphthylene.

For example, L₁ to L₃ can each independently be a single bond or any one group selected from the group consisting of the following groups:

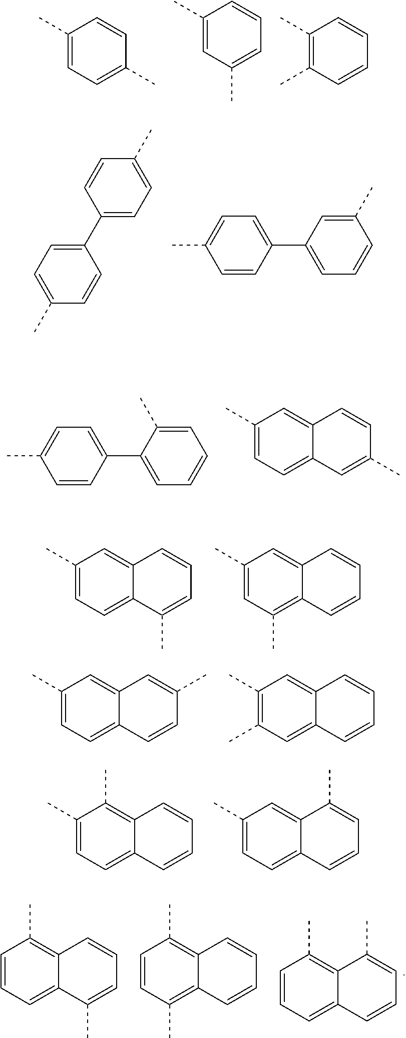

More preferably, L₁ to L₃ can each independently be a single bond, phenylene, or naphthylene. For example, L₁ can be a single bond, phenylene, or naphthylene, and L₂ and L₃ can each independently be a single bond or phenylene.

Specifically, in Chemical Formula 1 and Chemical Formulae 1-1 to 1-3, Ar₁ and Ar₂ can each independently be a substituted or unsubstituted C₆₋₂₀ aryl or substituted or unsubstituted C₂₋₂₀ heteroaryl containing at least one heteroatom selected from the group consisting of N, O and S.

More Specifically, Ar₁ and Ar₂ can each independently be phenyl, phenyl substituted with naphthyl, biphenyl, terphenyl, naphthyl, naphthyl substituted with phenyl, anthracenyl, phenanthrenyl, naphthacenyl, benzanthracenyl, chrysenyl, benzophenanthrenyl, pyrenyl, fluoranthenyl, triphenylenyl, perylenyl, dihydroindenyl, dibenzofuranyl, dibenzothiophenyl, benzonaphthofuranyl, or benzonaphthothiophenyl.

Preferably, Ar₁ and Ar₂ can each independently be phenyl, phenyl substituted with naphthyl, biphenyl, naphthyl, naphthyl substituted with phenyl, anthracenyl, phenanthrenyl, naphthacenyl, benzanthracenyl, chrysenyl, benzophenanthrenyl, pyrenyl, fluoranthenyl, dibenzofuranyl, or dibenzothiophenyl.

For example, Ar₁ and Ar₂ can each independently be any one group selected from the group consisting of the following groups:

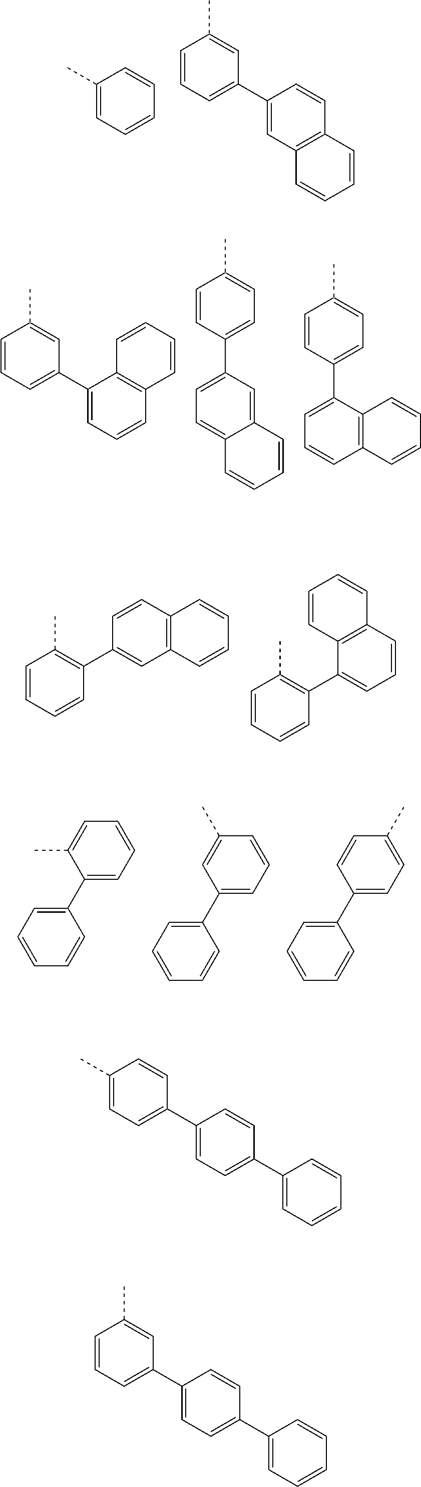

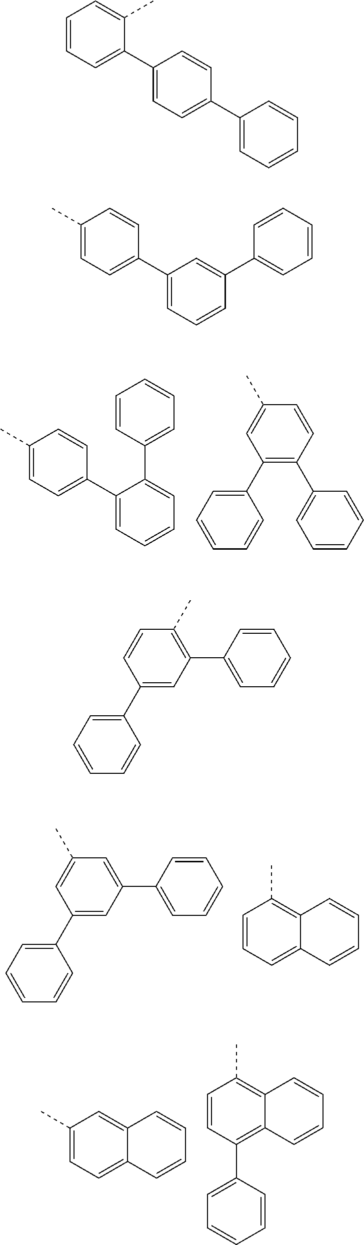

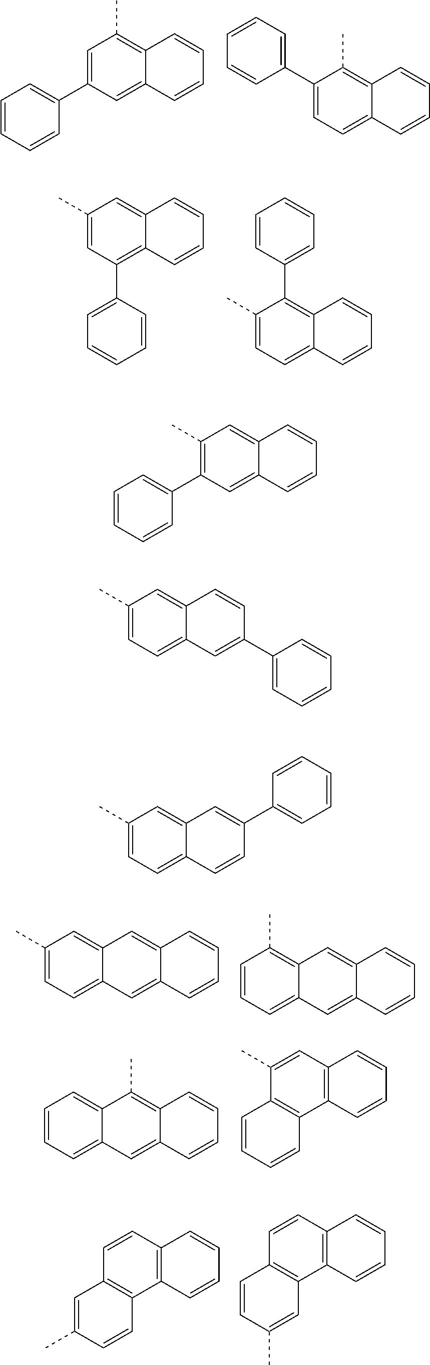

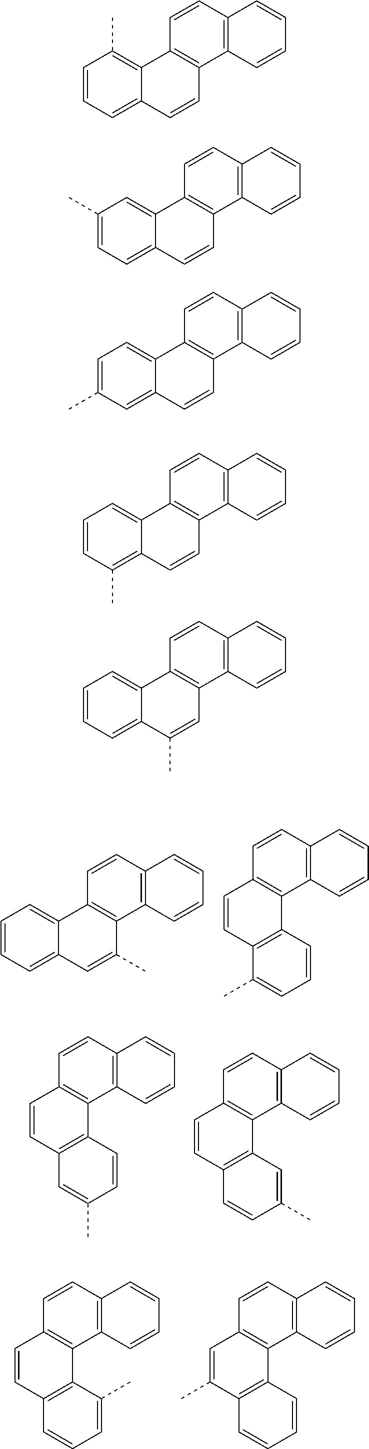

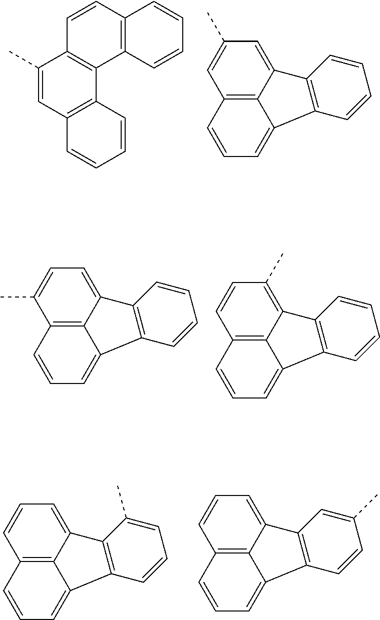

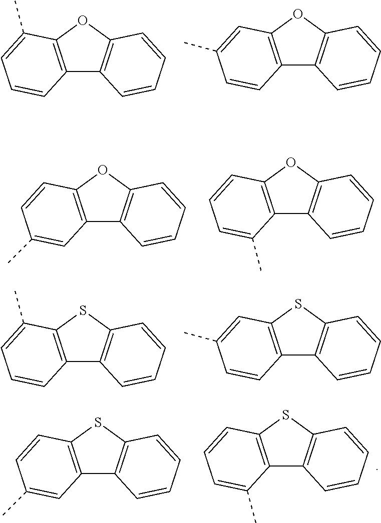

More preferably, Ar₁ and Ar₂ can each independently be phenyl, phenyl substituted with naphthyl, biphenyl, naphthyl, naphthyl substituted with phenyl, naphthacenyl, benzanthracenyl, chrysenyl, benzophenanthrenyl, pyrenyl, fluoranthenyl, dibenzofuranyl, or dibenzothiophenyl.

Specifically, Ar₁ and Ar₂ can each independently be phenyl, phenyl substituted with naphthyl, biphenyl, naphthyl, naphthyl substituted with phenyl, chrysenyl, benzophenanthrenyl, fluoranthenyl, dibenzofuranyl, or dibenzothiophenyl.

Further, one of Ar₁ and Ar₂ can be phenyl, phenyl substituted with naphthyl, biphenyl, naphthyl, naphthyl substituted with phenyl, dibenzofuranyl, or dibenzothiophenyl, and the other of Ar₁ and Ar₂ can be phenyl, phenyl substituted with naphthyl, biphenyl, naphthyl, naphthyl substituted with phenyl, chrysenyl, benzophenanthrenyl, or fluoranthenyl.

Meanwhile, in Chemical Formula 1 and Chemical Formulae 1-1 to 1-3, each Ar₃ independently can be hydrogen, deuterium, a substituted or unsubstituted C₆₋₂₀ aryl, or a substituted or unsubstituted C₂₋₂₀ heteroaryl containing at least one heteroatom selected from the group consisting of N, O and S.

Preferably, each Ar₃ independently can be hydrogen, deuterium, or a substituted or unsubstituted C₆₋₂₀ aryl.

Specifically, each Ar₃ independently can be hydrogen, deuterium, phenyl, phenyl substituted with naphthyl, biphenyl, terphenyl, naphthyl, naphthyl substituted with phenyl, anthracenyl, phenanthrenyl, naphthacenyl, benzanthracenyl, chrysenyl, benzophenanthrenyl, pyrenyl, fluoranthenyl, triphenylenyl, or perylenyl.

However, if Ar₃ is a substituted or unsubstituted C₁₆₋₆₀ aryl polycyclic aromatic ring or a C₁₆₋₂₀ aryl polycyclic aromatic ring, Ar₃ is not

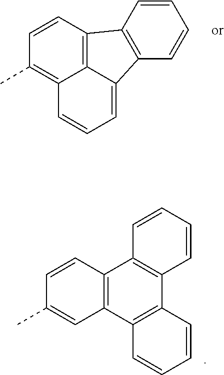

Preferably, each Ar₃ independently can be hydrogen, deuterium, phenyl, phenyl substituted with naphthyl, biphenyl, terphenyl, naphthyl, naphthyl substituted with phenyl, naphthacenyl, benzanthracenyl, chrysenyl, benzophenanthrenyl, pyrenyl, or fluoranthenyl.

For example, each Ar₃ independently can be hydrogen or deuterium, or any one group selected from the group consisting of the following groups:

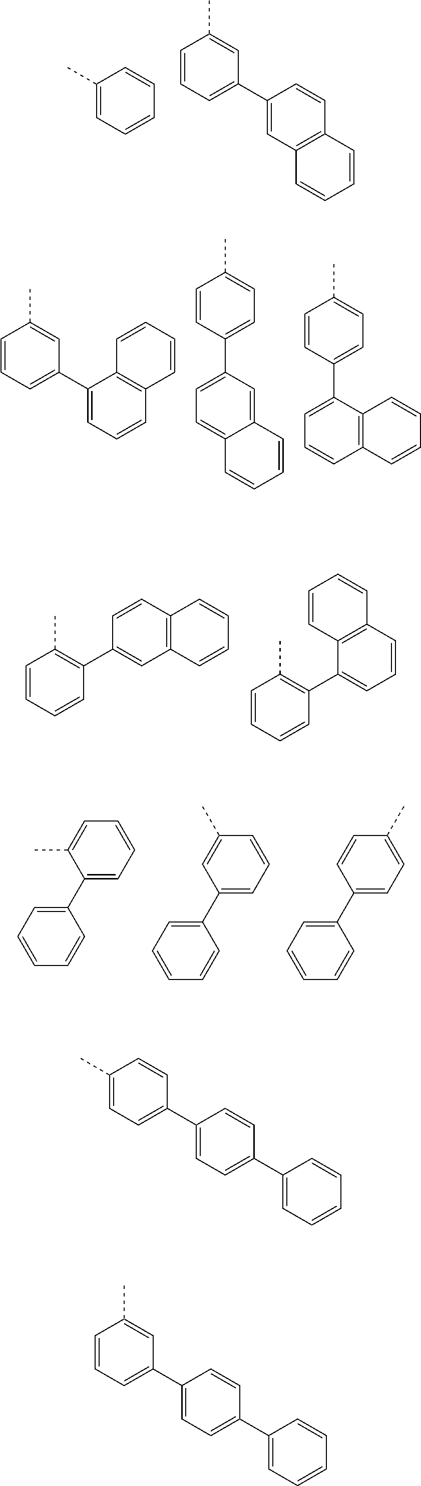

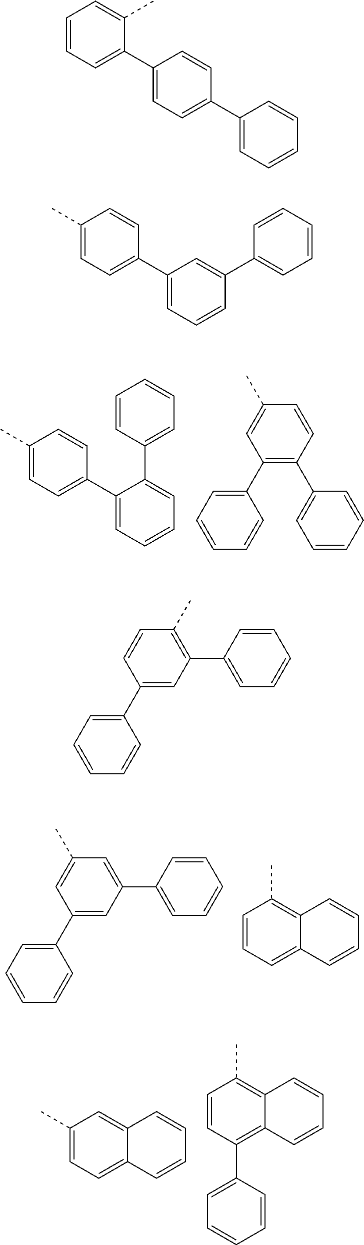

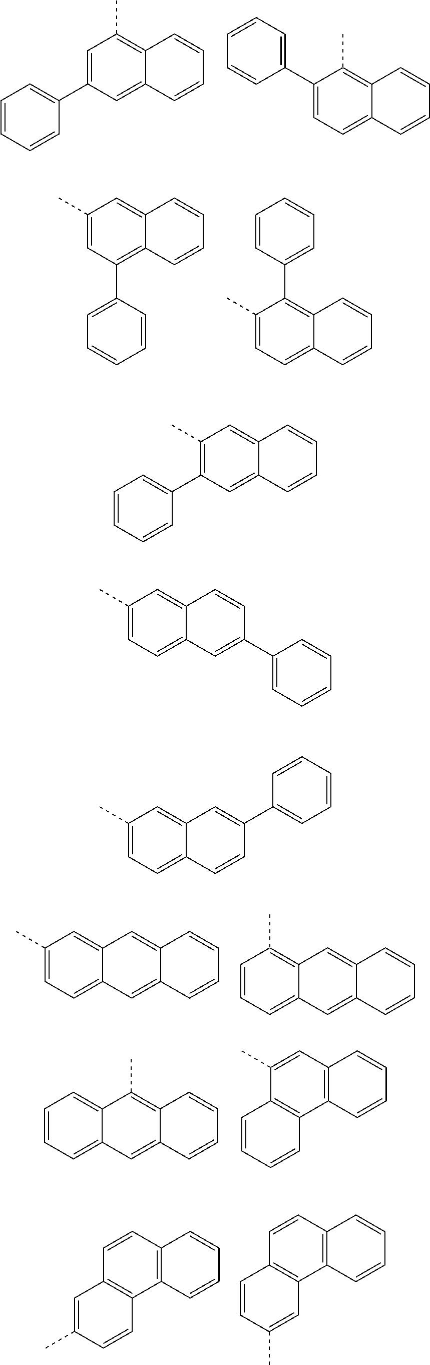

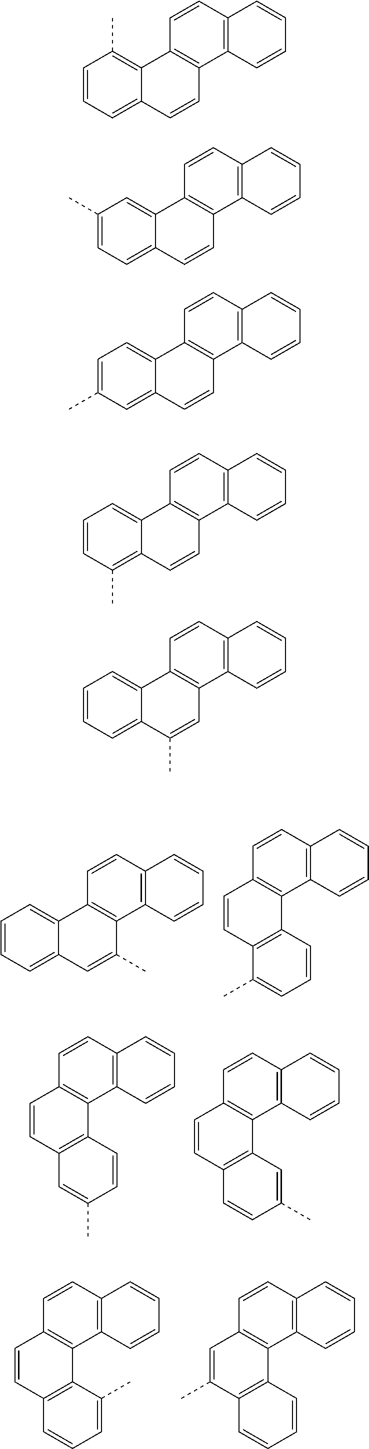

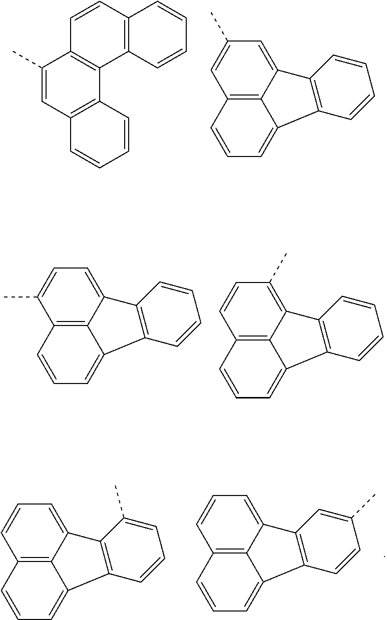

More preferably, each Ar₃ independently can be hydrogen, deuterium, phenyl, phenyl substituted with naphthyl, biphenyl, naphthyl, naphthyl substituted with phenyl, chrysenyl, benzophenanthrenyl, or fluoranthenyl.

In Chemical Formula 1, Chemical Formula 1-2, and Chemical Formula 1-3, n1, n2, n3 can be 0 or 1. Here, in Chemical Formula 1, when n1 is 0, it is a structure in which Ar₃ is not substituted but hydrogen is substituted in the dibenzofuran ring, which corresponds to Chemical Formula 1-1.

In addition, all hydrogens included in Chemical Formula 1 and Chemical Formulae 1-1 to 1-3 can each independently be replaced with deuterium.

Specifically, in Chemical Formula 1 and Chemical Formulae 1-1 to 1-3, at least one of Ar₁, Ar₂, and Ar₃ is a substituted or unsubstituted C₁₆₋₆₀ aryl polycyclic aromatic ring, preferably a substituted or unsubstituted C₁₆₋₆₀ aryl polynuclear aromatic ring having a structure in which three or more of benzene rings are fused. Here, the C₁₆₋₆₀ aryl polycyclic aromatic ring is a type of polynuclear aromatic hydrocarbons with a pair of common carbons sharing two carbon atoms of the benzene ring, referring to an aryl ring composed of 16 to 60 carbon atoms. For example, the C₁₆₋₆₀ aryl polycyclic aromatic ring has a structure in which three or more of benzene rings are fused, and is an aryl fused ring composed of 16 to 60 carbon atoms.

In the present disclosure, the compounds of Chemical Formula 1 and Chemical Formulae 1-1 to 1-3 have the advantageous feature of improving the efficiency, lowering the driving voltage, and/or enhancing the lifespan characteristics in organic light-emitting devices, by including a substituted or unsubstituted C₁₆₋₆₀ aryl polycyclic aromatic ring, for example, a substituted or unsubstituted C₁₆₋₆₀ aryl polycyclic aromatic ring in which three or more of benzene rings are fused, as one or more of Ar₁, Ar₂, and Ar₃, as mentioned above.

Specifically, at least one of Ar₁, Ar₂, and Ar₃ can be a C₁₆₋₃₀ aryl polycyclic aromatic ring, or C₁₆₋₂₄ aryl polycyclic aromatic ring, or C₁₆₋₂₀ aryl polycyclic aromatic ring.

Preferably, at least one of Ar₁, Ar₂ and Ar₃ can be naphthacenyl, benzanthracenyl, chrysenyl, benzophenanthrenyl, pyrenyl, fluoranthenyl, triphenylenyl, or perylenyl.

More preferably, at least one of Ar₁, Ar₂ and Ar₃ can be chrysenyl, benzophenanthrenyl, or fluoranthenyl.

For example, at least one of Ar₁, Ar₂ and Ar₃ can be any one group selected from the group consisting of the following groups:

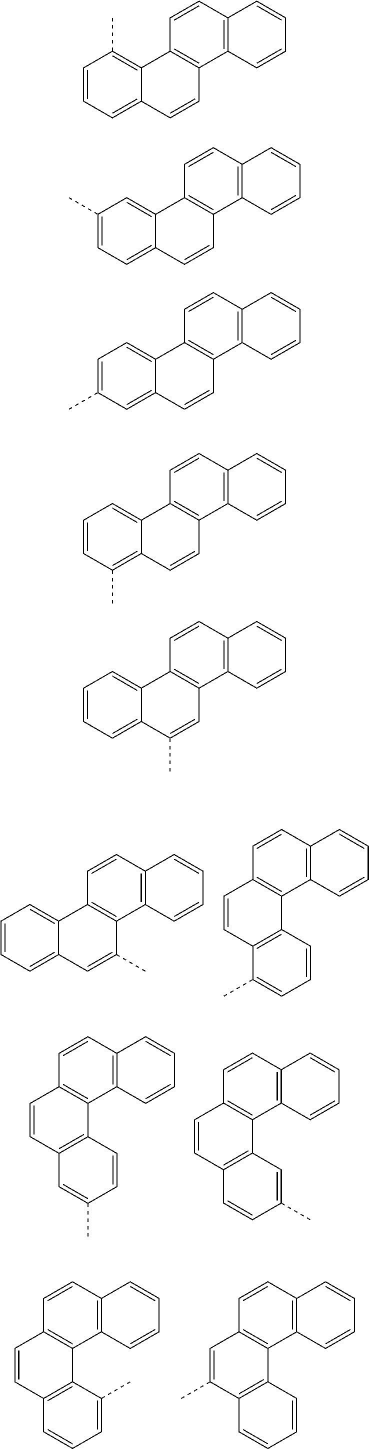

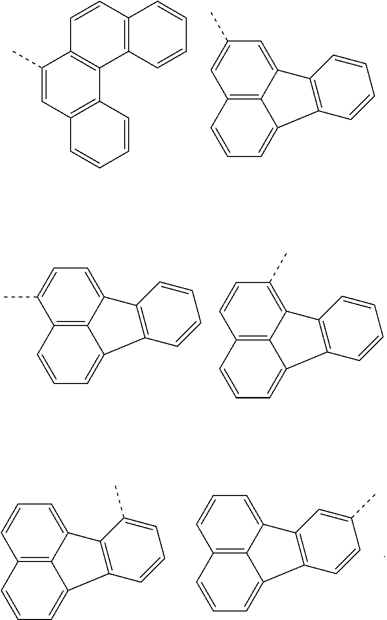

Meanwhile, if Ar₃ is an aromatic ring consisting of 16 to 60 carbon atoms in Chemical Formula 1, Chemical Formula 1-2, and Chemical Formula 1-3, Ar₃ is not

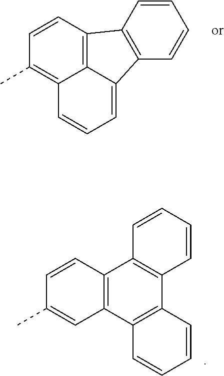

Specifically, if Ar₃ is fluoranthenyl or triphenylenyl in Chemical Formula 1, Chemical Formula 1-2, and Chemical Formula 1-3, Ar₃ is not

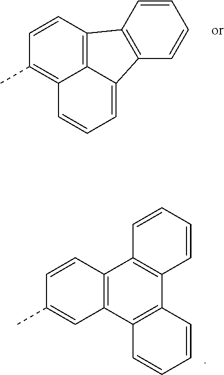

For example, one of Ar₁ and Ar₂ can be phenyl, phenyl substituted with naphthyl, biphenyl, naphthyl, naphthyl substituted with phenyl, dibenzofuranyl, or dibenzothiophenyl; the other of Ar₁ and Ar₂ can be phenyl, phenyl substituted with naphthyl, biphenyl, naphthyl, naphthyl substituted with phenyl, chrysenyl, benzophenanthrenyl, or fluoranthenyl; and each Ar₃ independently can be hydrogen, deuterium, phenyl, phenyl substituted with naphthyl, biphenyl, naphthyl, naphthyl substituted with phenyl, chrysenyl, benzophenanthrenyl, or fluoranthenyl.

More specifically, one of Ar₁ and Ar₂ can be phenyl, phenyl substituted with naphthyl, biphenyl, naphthyl, naphthyl substituted with phenyl, dibenzofuranyl, or dibenzothiophenyl; the other of Ar₁ and Ar₂ can be phenyl, phenyl substituted with naphthyl, biphenyl, naphthyl, or naphthyl substituted with phenyl; at least one of Ar₃ can be chrysenyl, benzophenanthrenyl, or fluoranthenyl; and the rest of Ar₃ can each independently be hydrogen, deuterium, phenyl, phenyl substituted with naphthyl, biphenyl, naphthyl, or naphthyl substituted with phenyl.

In addition, one of Ar₁ and Ar₂ can be phenyl, phenyl substituted with naphthyl, biphenyl, naphthyl, naphthyl substituted with phenyl, dibenzofuranyl, or dibenzothiophenyl; the other of Ar₁ and Ar₂ can be chrysenyl, benzophenanthrenyl, or fluoranthenyl; and each Ar₃ independently can be hydrogen, deuterium, phenyl, phenyl substituted with naphthyl, biphenyl, naphthyl, or naphthyl substituted with phenyl.

Meanwhile, all hydrogens included in Chemical Formula 1 and Chemical Formulae 1-1 to 1-3 can each independently be replaced with deuterium (D).

Representative examples of the compound of Chemical Formula 1 are as follows:

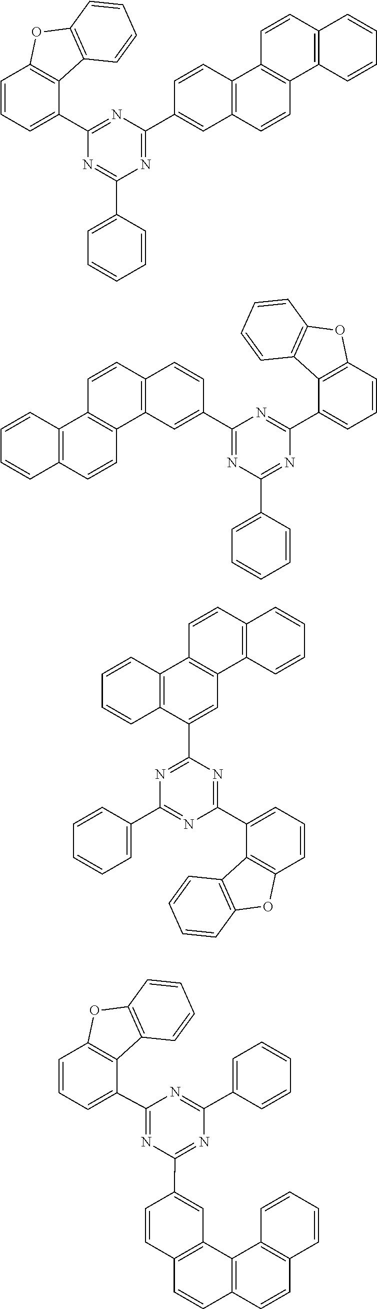

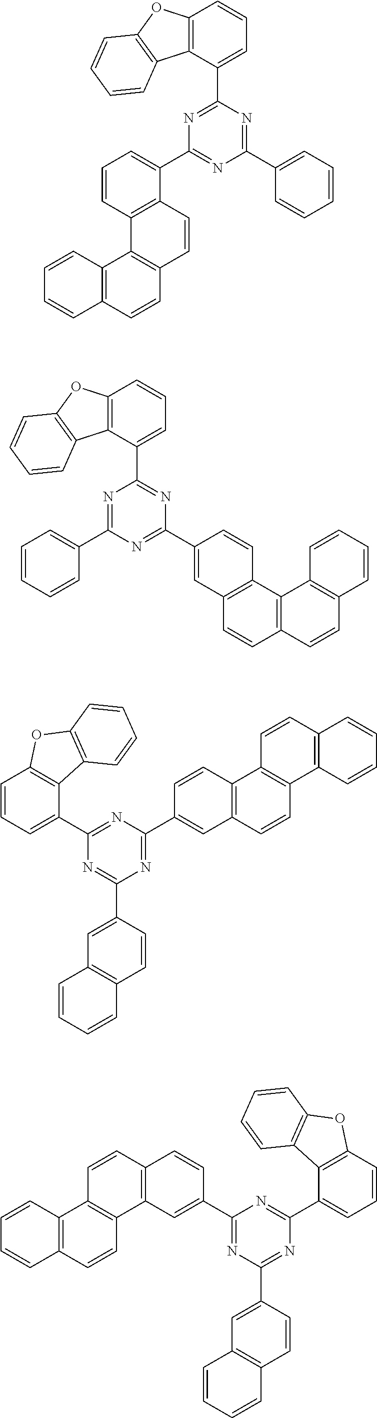 [SMILES: c1ccc(-c2nc(-c3cccc4c3ccc3ccc5ccccc5c34)nc(-c3cccc4oc5ccccc5c34)n2)cc1 ; c1ccc(-c2nc(-c3ccc4c(ccc5ccc6ccccc6c54)c3)nc(-c3cccc4oc5ccccc5c34)n2)cc1 ; c1ccc2cc(-c3nc(-c4ccc5c(ccc6c7ccccc7ccc56)c4)nc(-c4cccc5oc6ccccc6c45)n3)ccc2c1 ; c1ccc2cc(-c3nc(-c4ccc5ccc6c7ccccc7ccc6c5c4)nc(-c4cccc5oc6ccccc6c45)n3)ccc2c1]

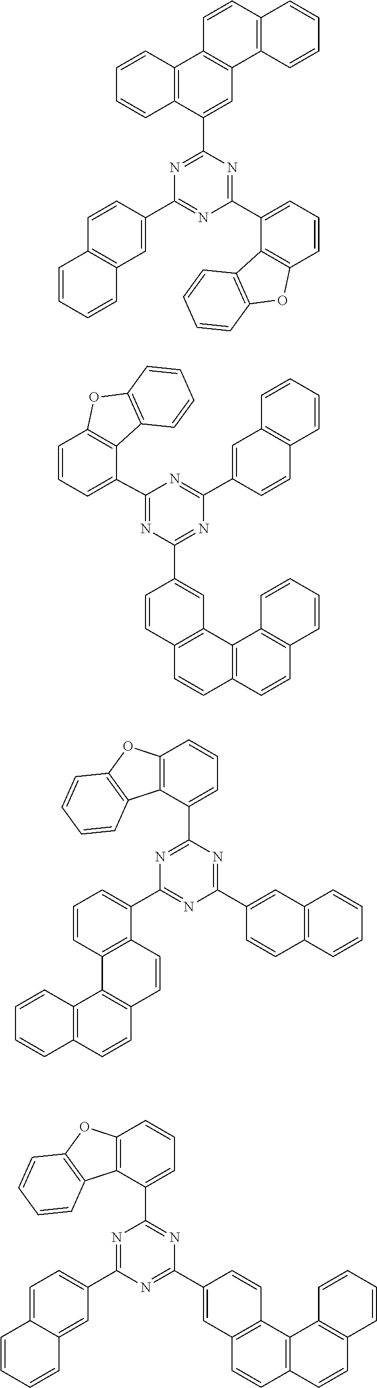

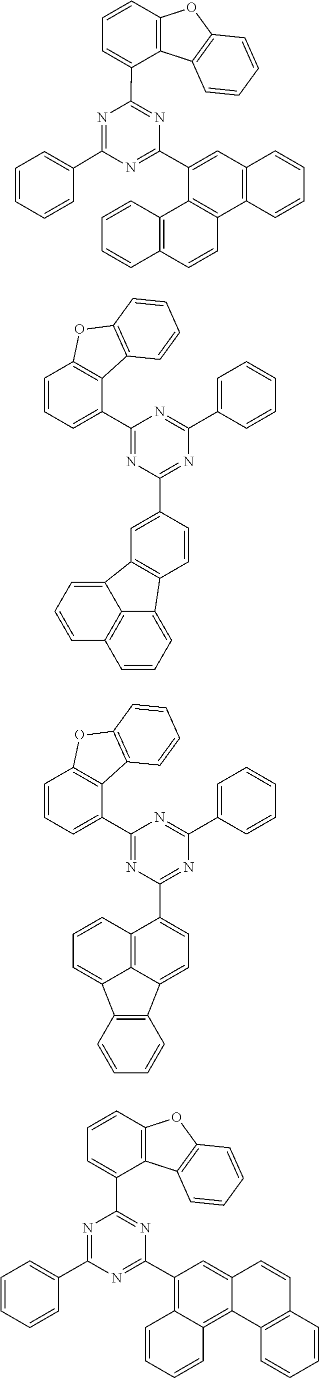

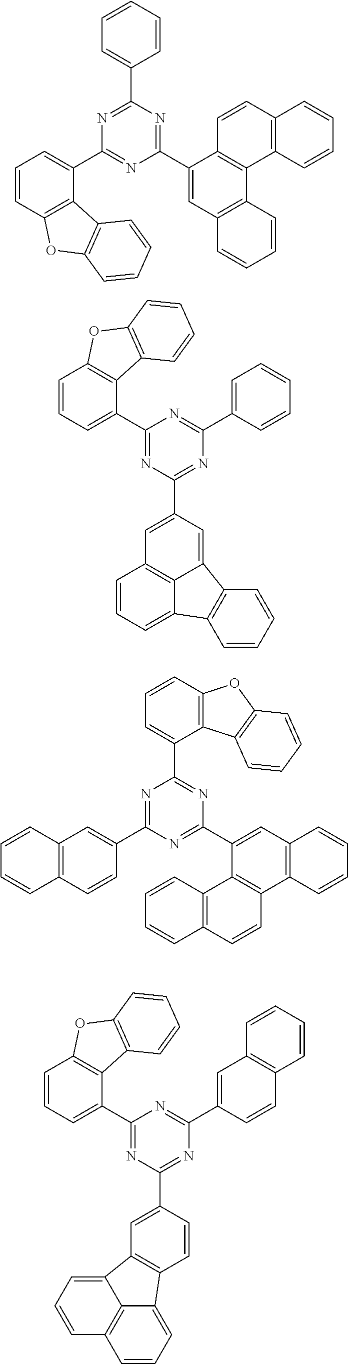

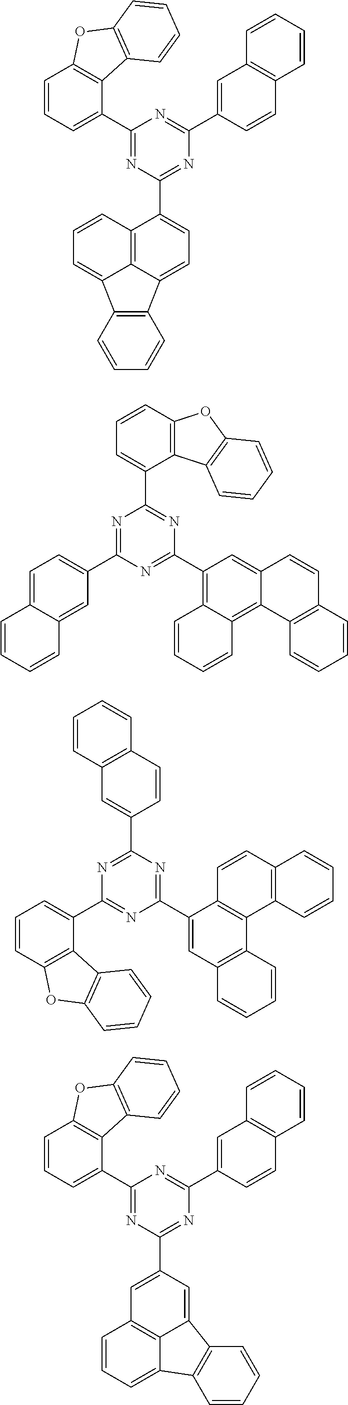

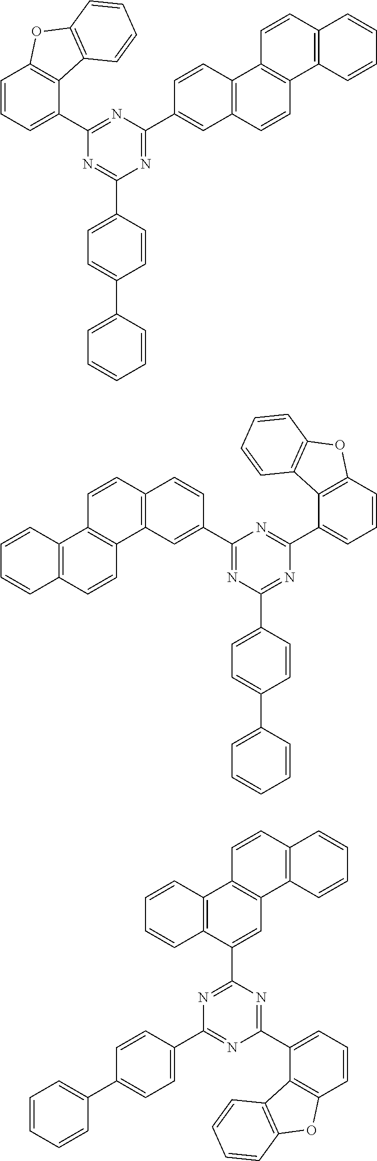

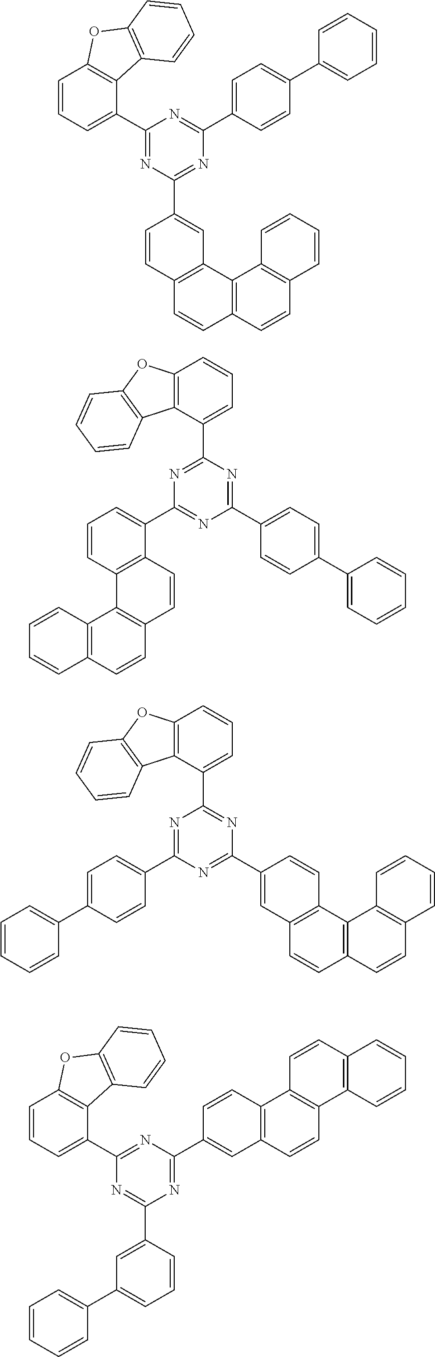

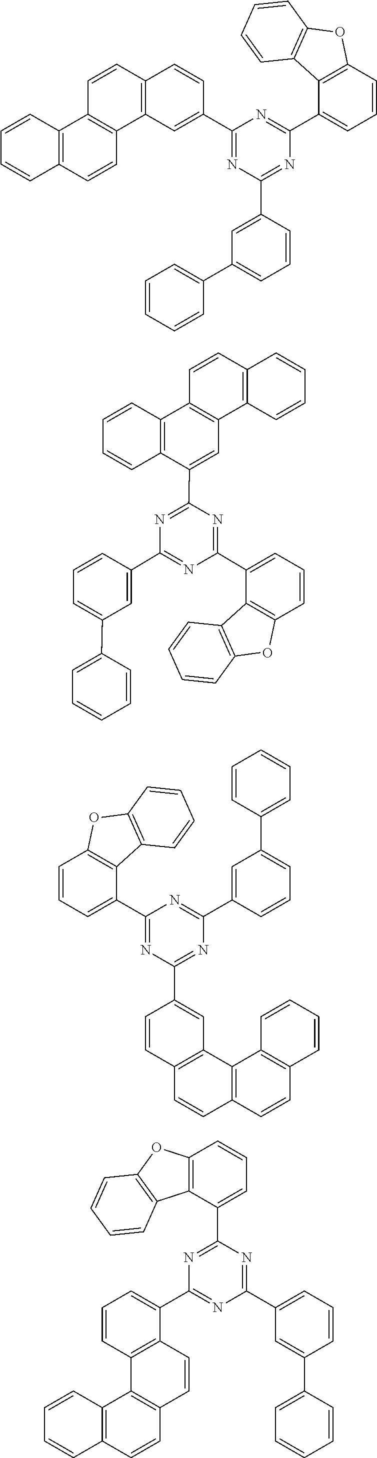

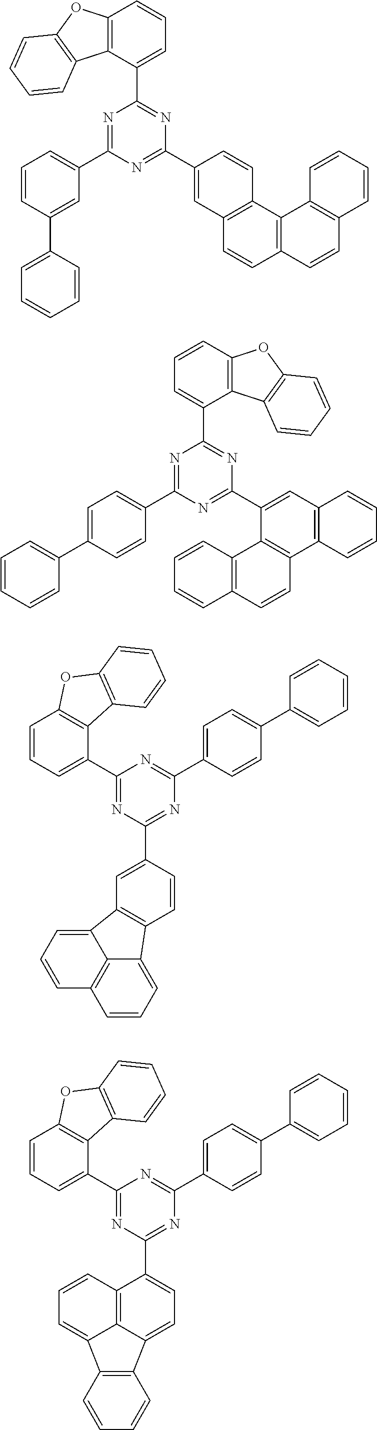

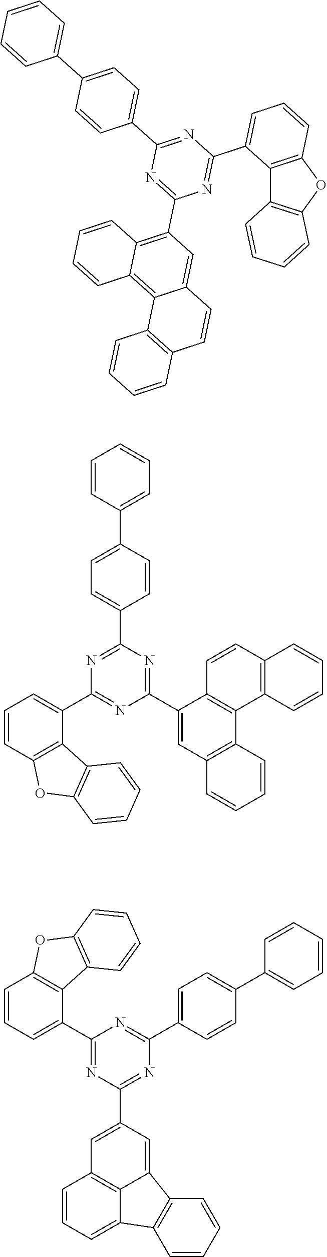 [SMILES: c1ccc(-c2ccc(-c3nc(-c4cc5ccc6ccccc6c5c5ccccc45)nc(-c4cccc5oc6ccccc6c45)n3)cc2)cc1 ; c1ccc(-c2ccc(-c3nc(-c4cc5ccccc5c5c4ccc4ccccc45)nc(-c4cccc5oc6ccccc6c45)n3)cc2)cc1 ; c1ccc(-c2ccc(-c3nc(-c4cc5c6c(cccc6c4)-c4ccccc4-5)nc(-c4cccc5oc6ccccc6c45)n3)cc2)cc1]

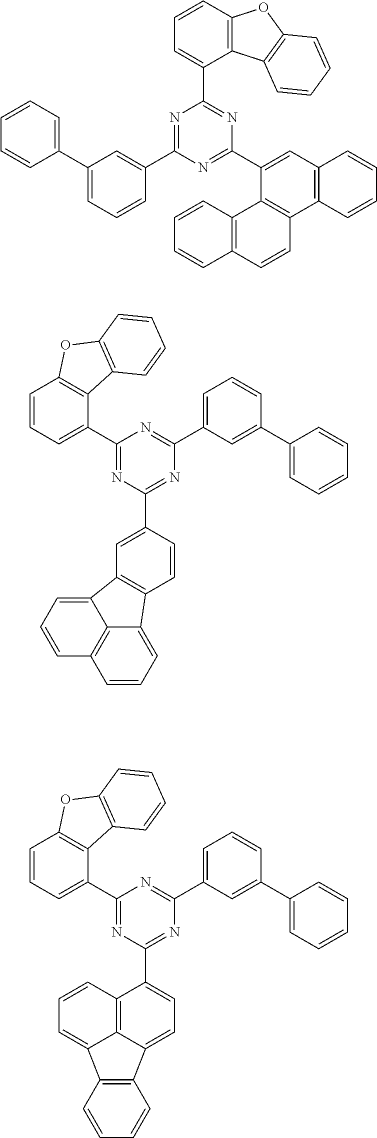 [SMILES: c1ccc(-c2cccc(-c3nc(-c4cccc5oc6ccccc6c45)nc(-c4cc5ccccc5c5ccc6ccccc6c45)n3)c2)cc1 ; c1ccc(-c2cccc(-c3nc(-c4ccc5c(c4)-c4cccc6cccc-5c46)nc(-c4cccc5oc6ccccc6c45)n3)c2)cc1 ; c1ccc(-c2cccc(-c3nc(-c4ccc5c6c(cccc46)-c4ccccc4-5)nc(-c4cccc5oc6ccccc6c45)n3)c2)cc1]

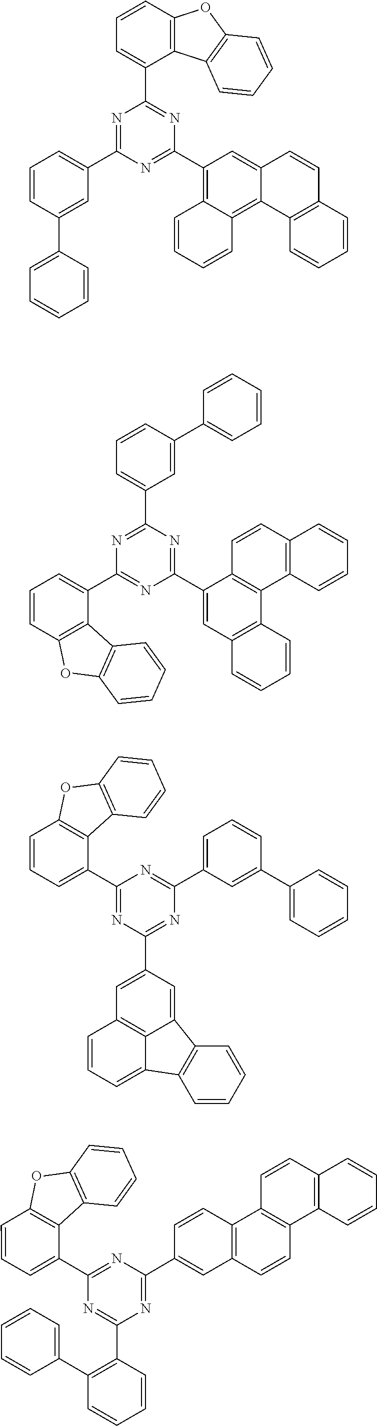 [SMILES: c1ccc(-c2cccc(-c3nc(-c4cc5ccc6ccccc6c5c5ccccc45)nc(-c4cccc5oc6ccccc6c45)n3)c2)cc1 ; c1ccc(-c2cccc(-c3nc(-c4cc5ccccc5c5c4ccc4ccccc45)nc(-c4cccc5oc6ccccc6c45)n3)c2)cc1 ; c1ccc(-c2cccc(-c3nc(-c4cc5c6c(cccc6c4)-c4ccccc4-5)nc(-c4cccc5oc6ccccc6c45)n3)c2)cc1 ; c1ccc(-c2ccccc2-c2nc(-c3ccc4c(ccc5c6ccccc6ccc45)c3)nc(-c3cccc4oc5ccccc5c34)n2)cc1]

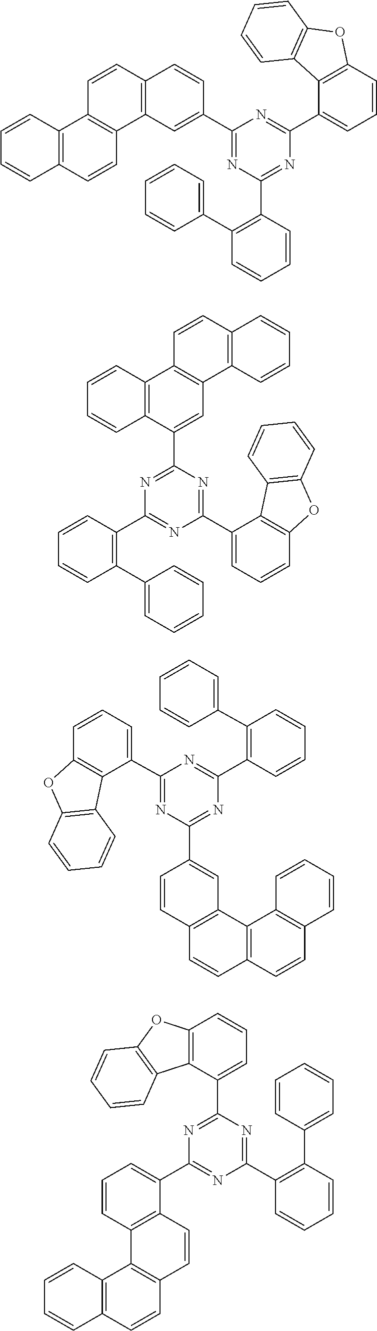

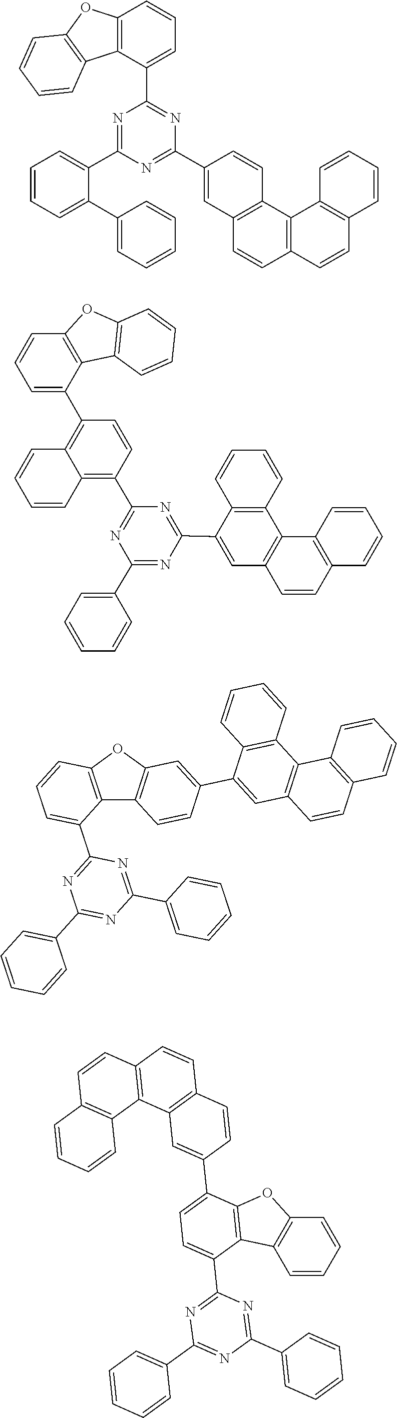

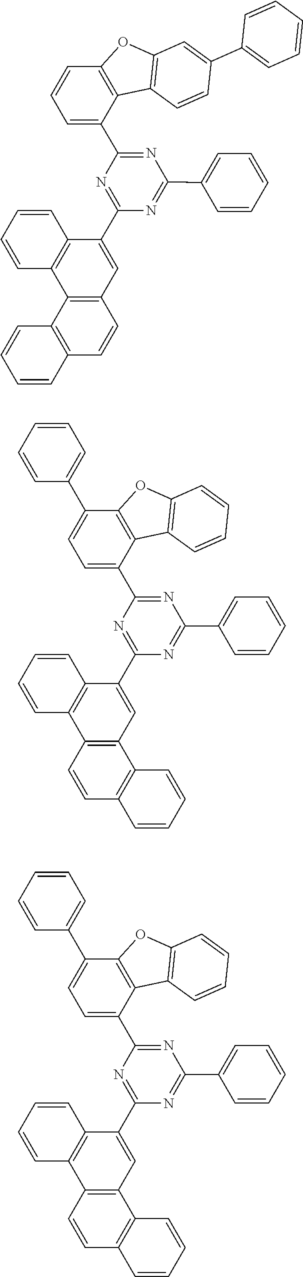

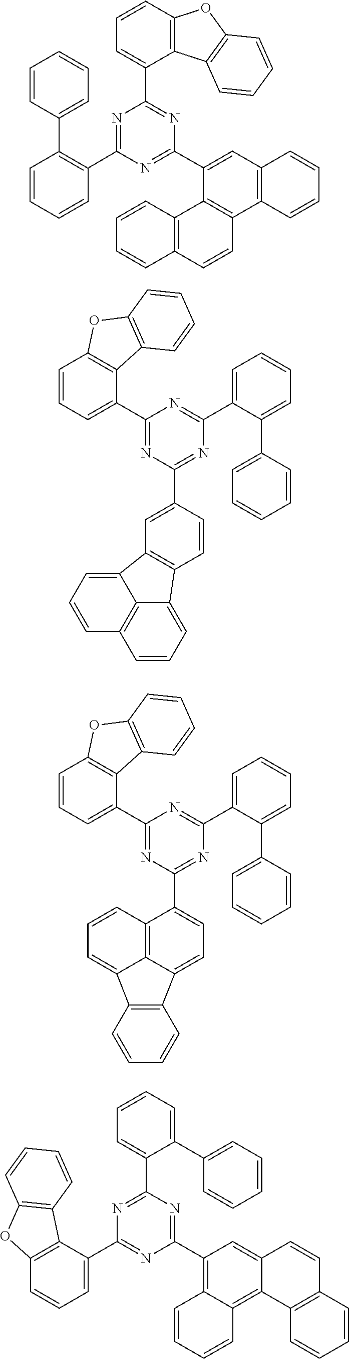

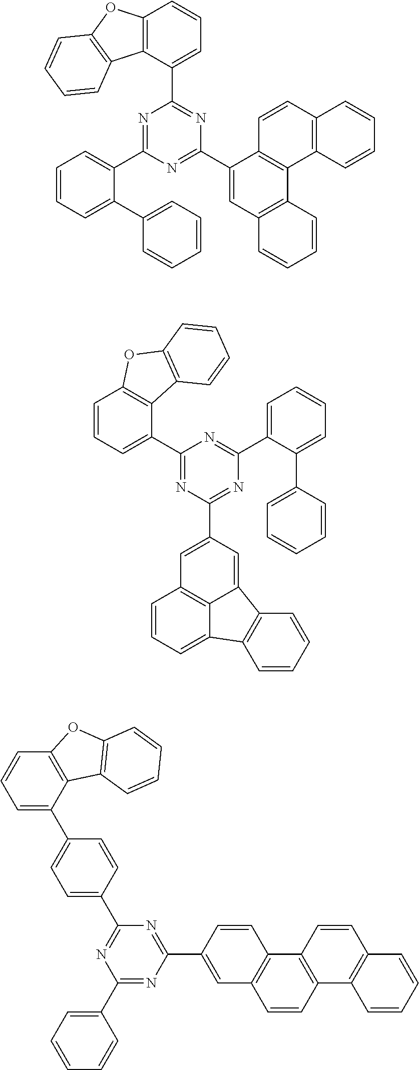 [SMILES: c1ccc(-c2ccccc2-c2nc(-c3cc4ccccc4c4c3ccc3ccccc34)nc(-c3cccc4oc5ccccc5c34)n2)cc1 ; c1ccc(-c2ccccc2-c2nc(-c3cc4c5c(cccc5c3)-c3ccccc3-4)nc(-c3cccc4oc5ccccc5c34)n2)cc1 ; c1ccc(-c2nc(-c3ccc(-c4cccc5oc6ccccc6c45)cc3)nc(-c3ccc4c(ccc5c6ccccc6ccc45)c3)n2)cc1]

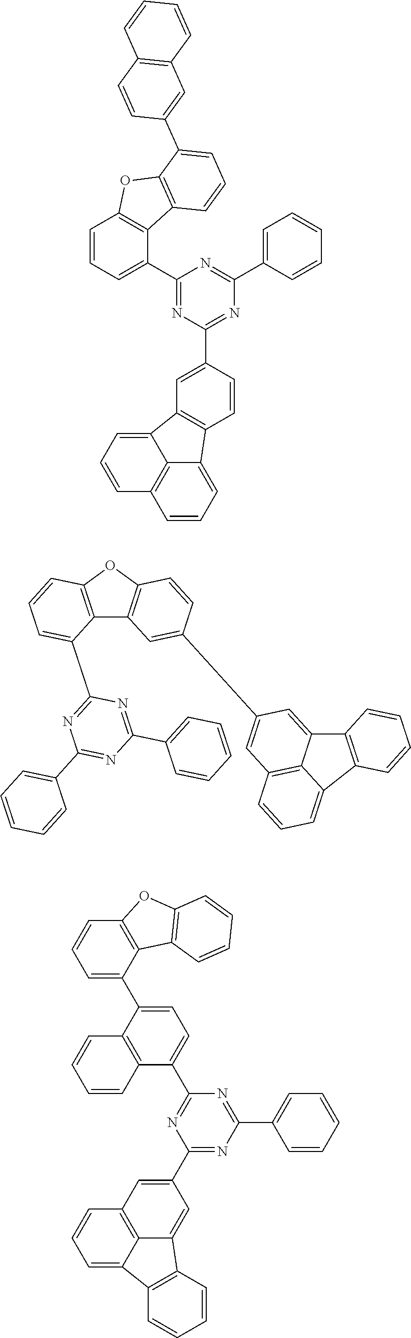 [SMILES: c1ccc(-c2nc(-c3ccc4c(c3)-c3cccc5cccc-4c35)nc(-c3cccc4oc5c(-c6ccc7ccccc7c6)cccc5c34)n2)cc1 ; c1ccc(-c2nc(-c3ccccc3)nc(-c3cccc4oc5ccc(-c6cc7c8c(cccc8c6)-c6ccccc6-7)cc5c34)n2)cc1 ; c1ccc(-c2nc(-c3cc4c5c(cccc5c3)-c3ccccc3-4)nc(-c3ccc(-c4cccc5oc6ccccc6c45)c4ccccc34)n2)cc1]

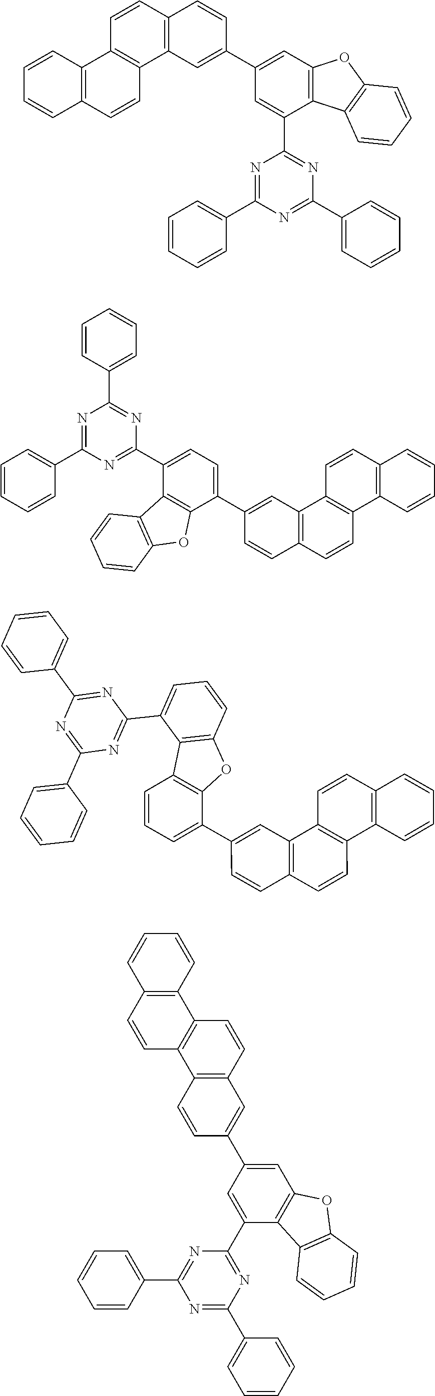 [SMILES: c1ccc(-c2nc(-c3ccccc3)nc(-c3cc(-c4ccc5ccc6c7ccccc7ccc6c5c4)cc4oc5ccccc5c34)n2)cc1 ; c1ccc(-c2nc(-c3ccccc3)nc(-c3ccc(-c4ccc5ccc6c7ccccc7ccc6c5c4)c4oc5ccccc5c34)n2)cc1 ; c1ccc(-c2nc(-c3ccccc3)nc(-c3cccc4oc5c(-c6ccc7ccc8c9ccccc9ccc8c7c6)cccc5c34)n2)cc1 ; c1ccc(-c2nc(-c3ccccc3)nc(-c3cc(-c4ccc5c(ccc6c7ccccc7ccc56)c4)cc4oc5ccccc5c34)n2)cc1]

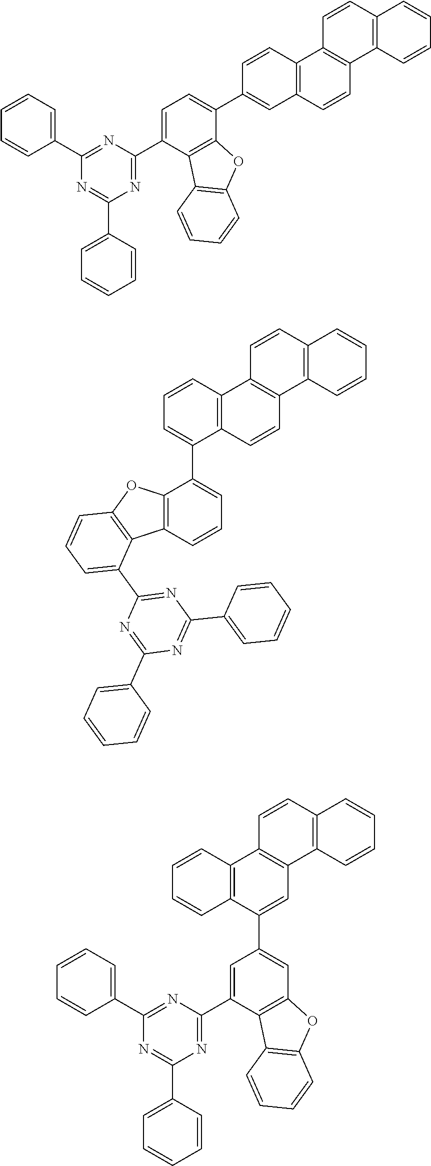 [SMILES: c1ccc(-c2nc(-c3ccccc3)nc(-c3ccc(-c4ccc5c(ccc6c7ccccc7ccc56)c4)c4oc5ccccc5c34)n2)cc1 ; c1ccc(-c2nc(-c3ccccc3)nc(-c3cccc4oc5c(-c6cccc7c6ccc6c8ccccc8ccc76)cccc5c34)n2)cc1 ; c1ccc(-c2nc(-c3ccccc3)nc(-c3cc(-c4cc5c6ccccc6ccc5c5ccccc45)cc4oc5ccccc5c34)n2)cc1]

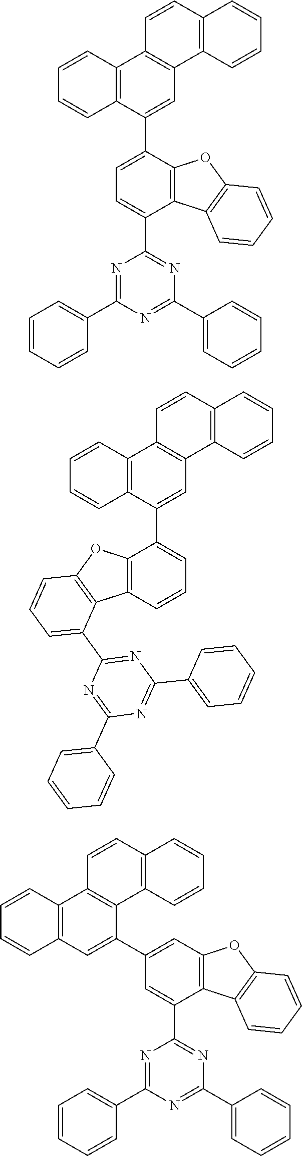

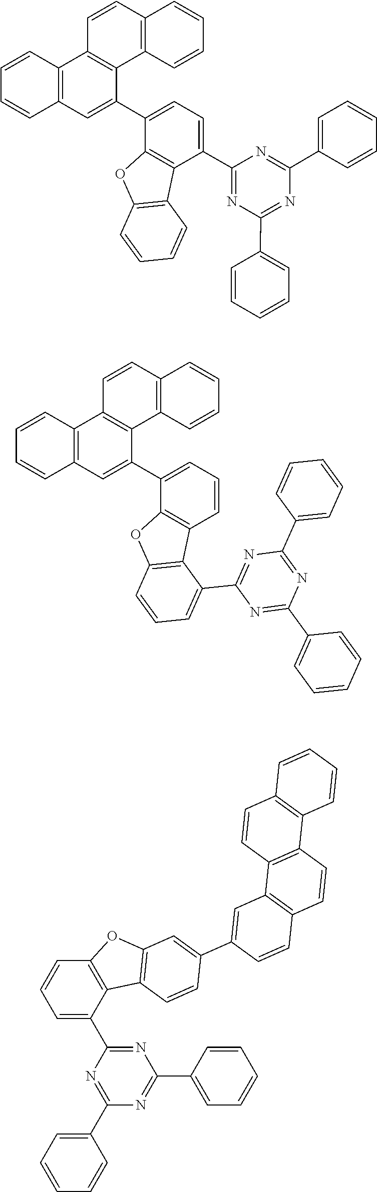 [SMILES: c1ccc(-c2nc(-c3ccccc3)nc(-c3ccc(-c4cc5ccccc5c5ccc6ccccc6c45)c4oc5ccccc5c34)n2)cc1 ; c1ccc(-c2nc(-c3ccccc3)nc(-c3cccc4oc5c(-c6cc7ccccc7c7ccc8ccccc8c67)cccc5c34)n2)cc1 ; c1ccc(-c2nc(-c3ccccc3)nc(-c3cccc4oc5cc(-c6ccc7ccc8c9ccccc9ccc8c7c6)ccc5c34)n2)cc1]

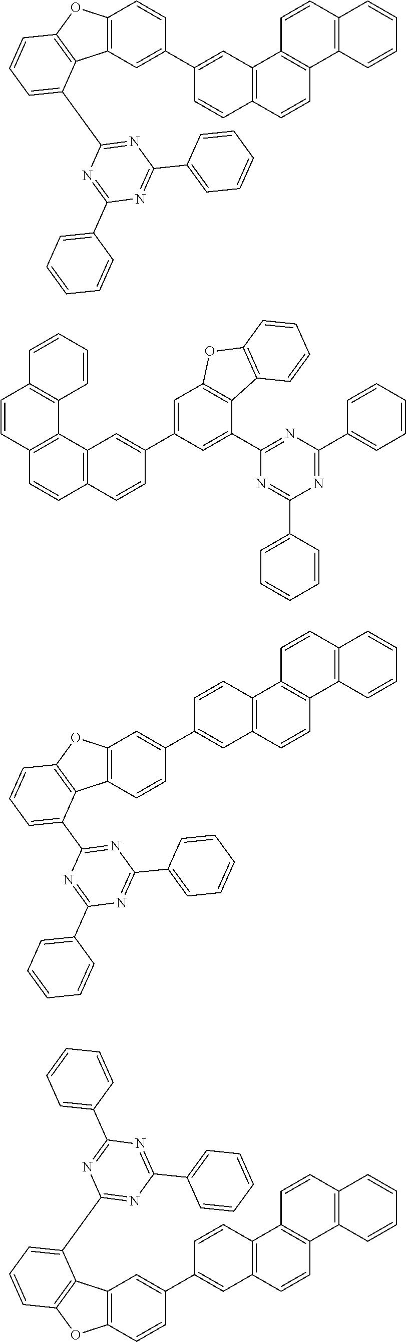

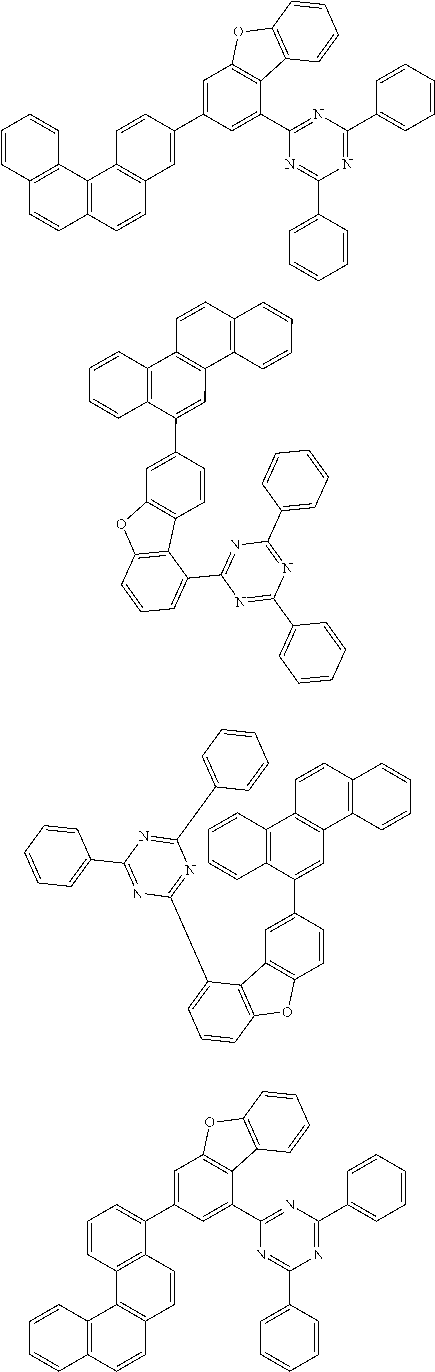

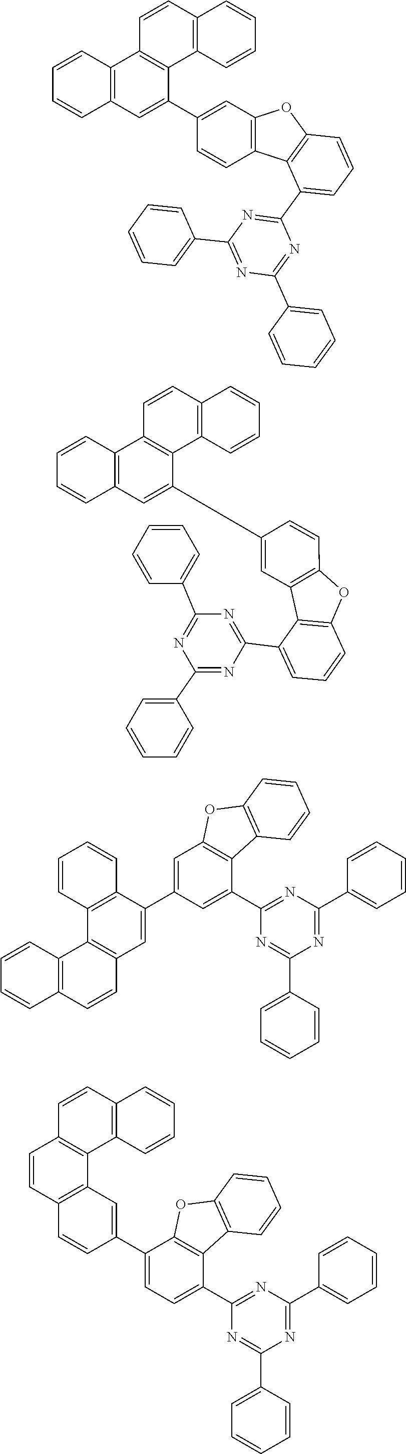

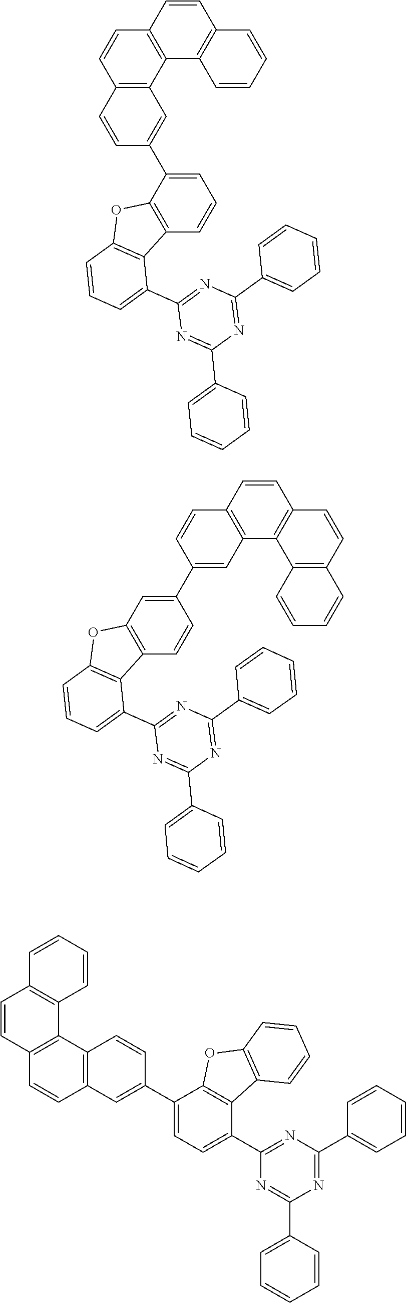

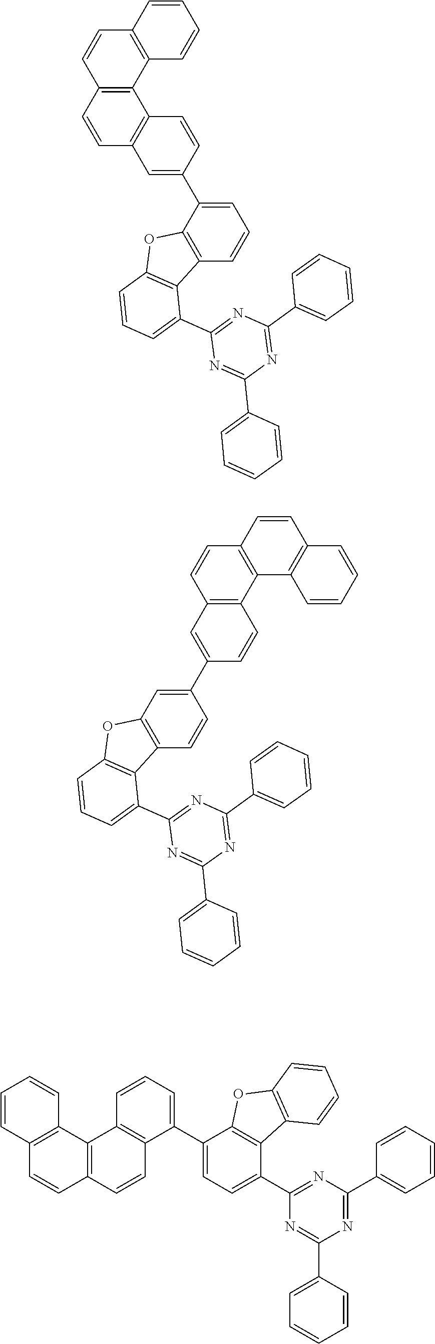

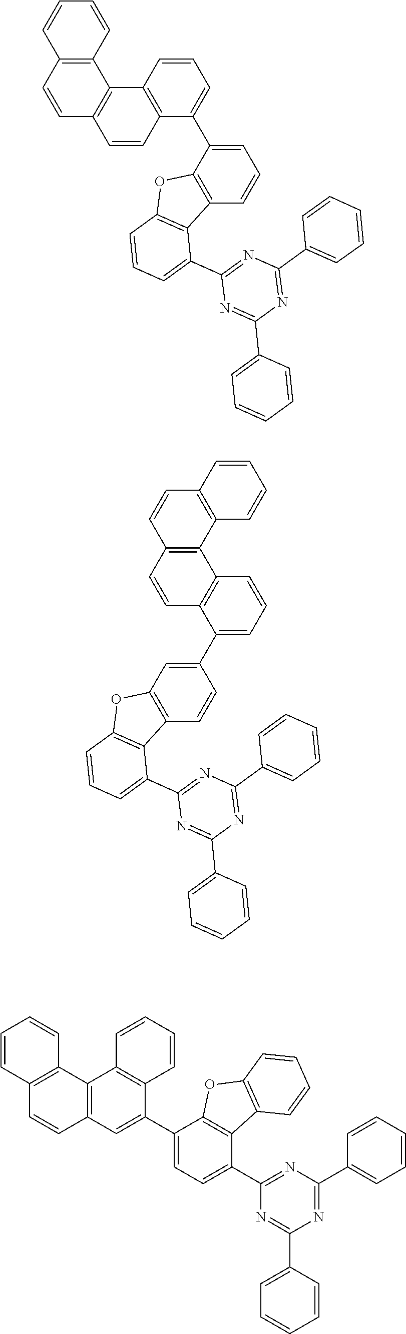

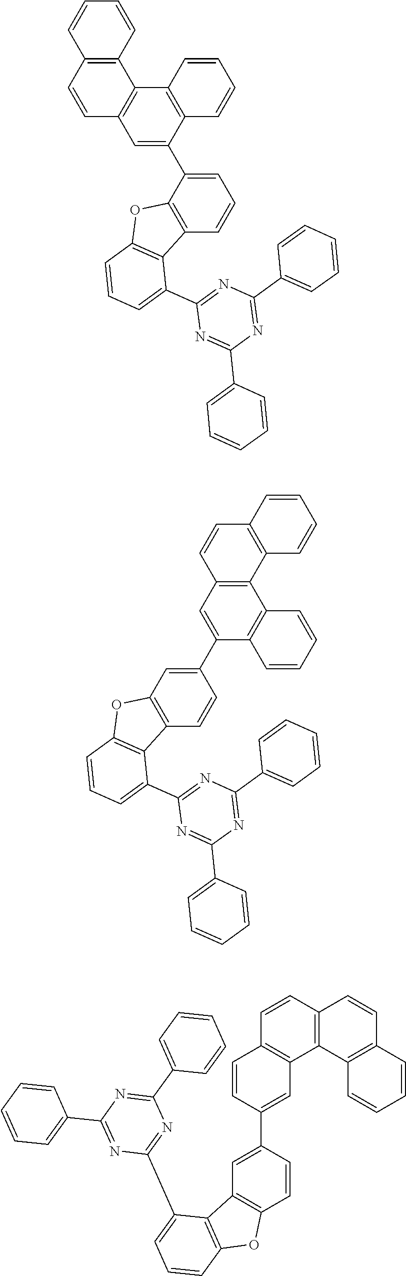 [SMILES: c1ccc(-c2nc(-c3ccccc3)nc(-c3cccc4oc5c(-c6cc7ccc8ccccc8c7c7ccccc67)cccc5c34)n2)cc1 ; c1ccc(-c2nc(-c3ccccc3)nc(-c3cccc4oc5cc(-c6cc7ccc8ccccc8c7c7ccccc67)ccc5c34)n2)cc1 ; c1ccc(-c2nc(-c3ccccc3)nc(-c3cccc4oc5ccc(-c6ccc7ccc8ccc9ccccc9c8c7c6)cc5c34)n2)cc1]

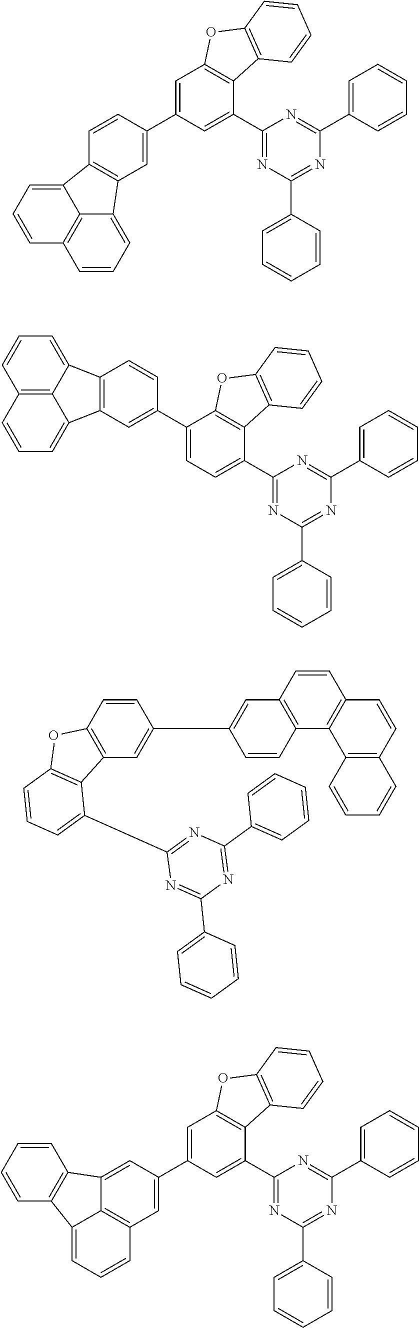

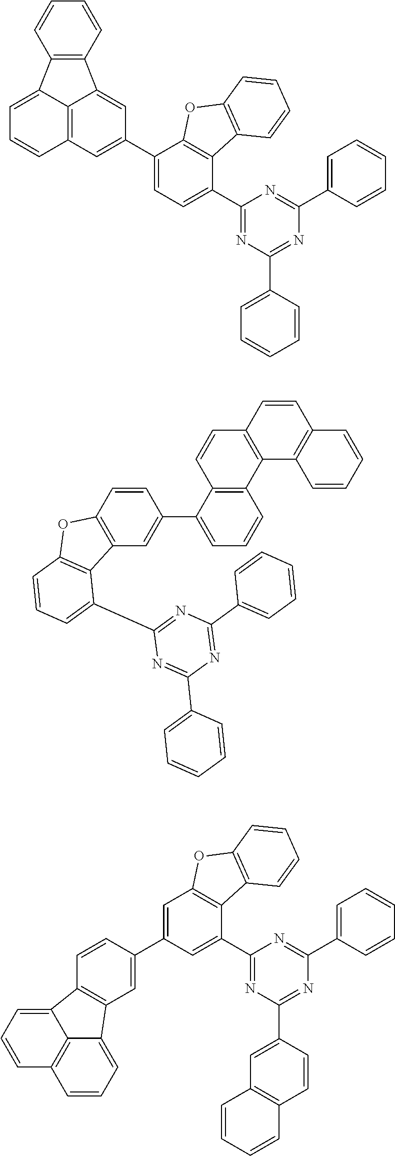

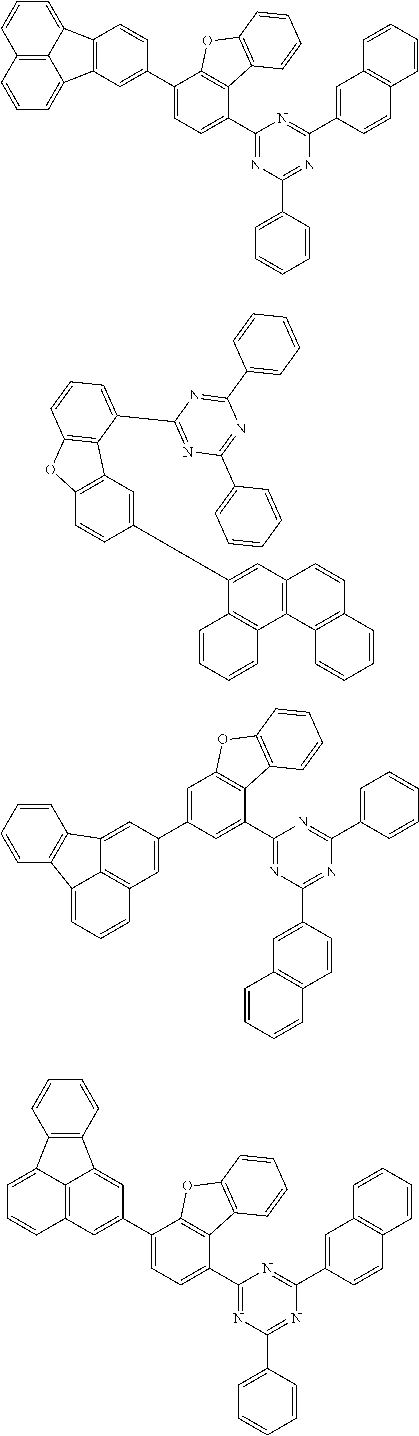

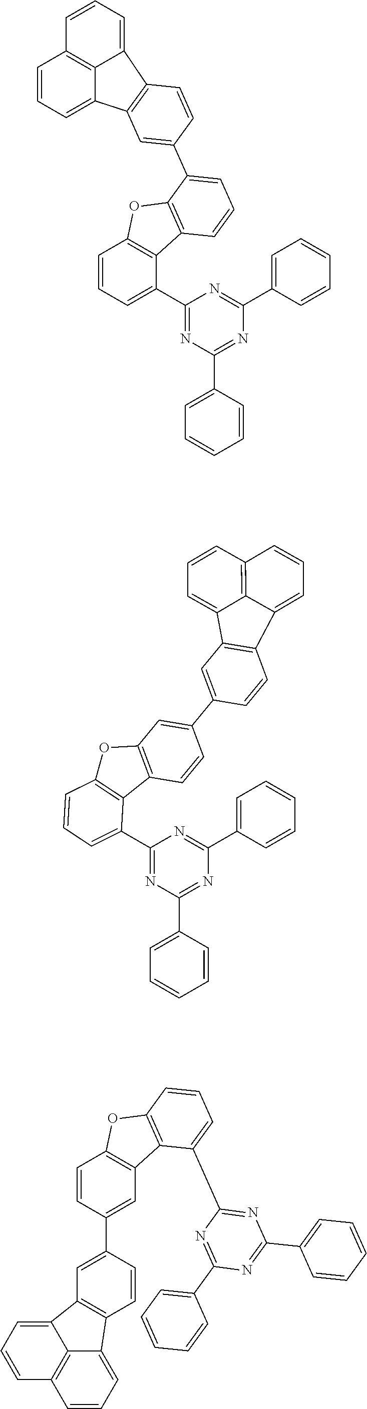 [SMILES: c1ccc(-c2nc(-c3ccccc3)nc(-c3cccc4oc5c(-c6ccc7c(c6)-c6cccc8cccc-7c68)cccc5c34)n2)cc1 ; c1ccc(-c2nc(-c3ccccc3)nc(-c3cccc4oc5cc(-c6ccc7c(c6)-c6cccc8cccc-7c68)ccc5c34)n2)cc1 ; c1ccc(-c2nc(-c3ccccc3)nc(-c3cccc4oc5ccc(-c6ccc7c(c6)-c6cccc8cccc-7c68)cc5c34)n2)cc1]

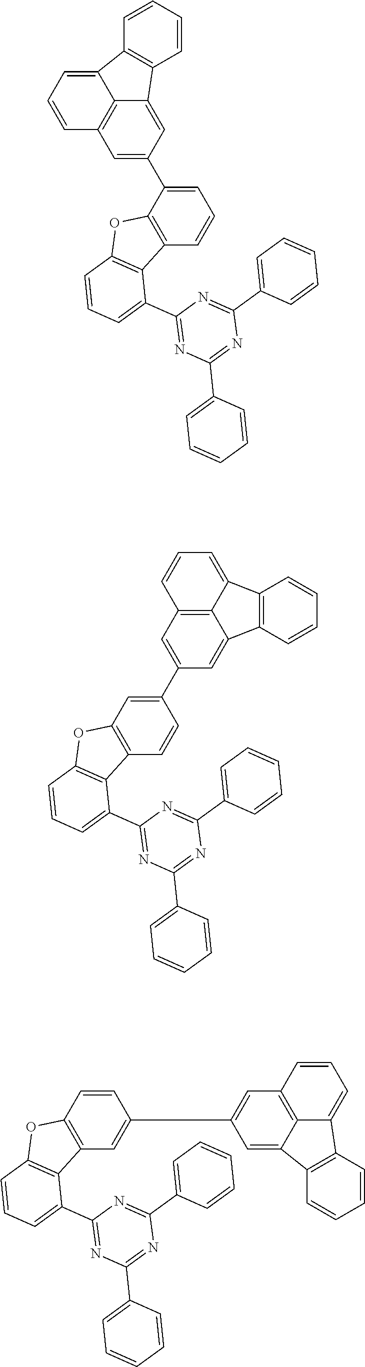 [SMILES: c1ccc(-c2nc(-c3ccccc3)nc(-c3cccc4oc5c(-c6cc7c8c(cccc8c6)-c6ccccc6-7)cccc5c34)n2)cc1 ; c1ccc(-c2nc(-c3ccccc3)nc(-c3cccc4oc5cc(-c6cc7c8c(cccc8c6)-c6ccccc6-7)ccc5c34)n2)cc1 ; c1ccc(-c2nc(-c3ccccc3)nc(-c3cccc4oc5ccc(-c6cc7c8c(cccc8c6)-c6ccccc6-7)cc5c34)n2)cc1]

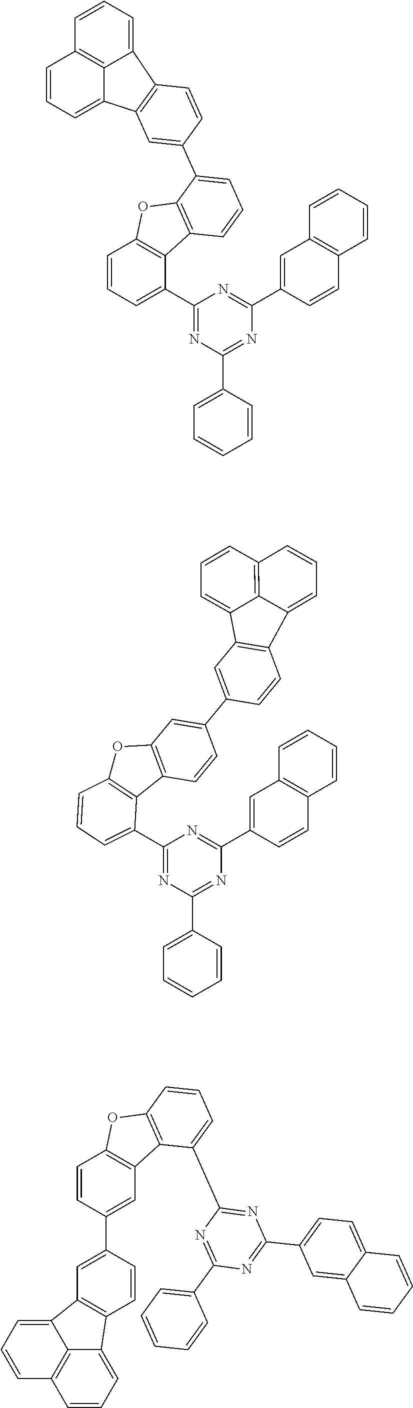 [SMILES: c1ccc(-c2nc(-c3ccc4ccccc4c3)nc(-c3cccc4oc5c(-c6ccc7c(c6)-c6cccc8cccc-7c68)cccc5c34)n2)cc1 ; c1ccc(-c2nc(-c3ccc4ccccc4c3)nc(-c3cccc4oc5cc(-c6ccc7c(c6)-c6cccc8cccc-7c68)ccc5c34)n2)cc1 ; c1ccc(-c2nc(-c3ccc4ccccc4c3)nc(-c3cccc4oc5ccc(-c6ccc7c(c6)-c6cccc8cccc-7c68)cc5c34)n2)cc1]

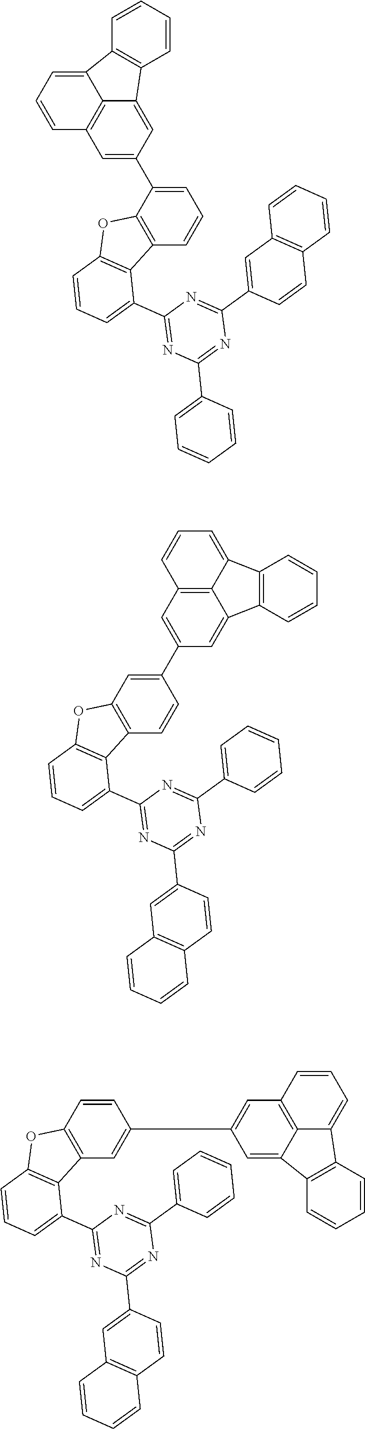 [SMILES: c1ccc(-c2nc(-c3ccc4ccccc4c3)nc(-c3cccc4oc5c(-c6cc7c8c(cccc8c6)-c6ccccc6-7)cccc5c34)n2)cc1 ; c1ccc(-c2nc(-c3ccc4ccccc4c3)nc(-c3cccc4oc5cc(-c6cc7c8c(cccc8c6)-c6ccccc6-7)ccc5c34)n2)cc1 ; c1ccc(-c2nc(-c3ccc4ccccc4c3)nc(-c3cccc4oc5ccc(-c6cc7c8c(cccc8c6)-c6ccccc6-7)cc5c34)n2)cc1]

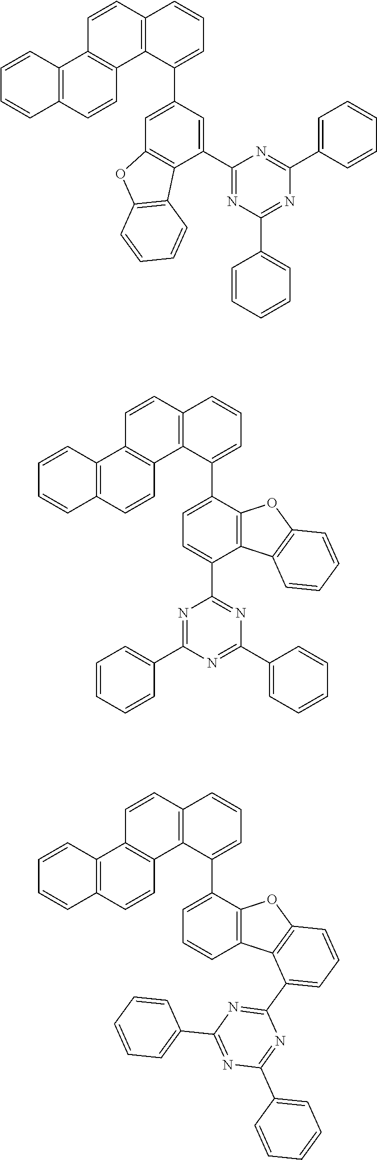 [SMILES: c1ccc(-c2nc(-c3ccccc3)nc(-c3cc(-c4cccc5ccc6c7ccccc7ccc6c45)cc4oc5ccccc5c34)n2)cc1 ; c1ccc(-c2nc(-c3ccccc3)nc(-c3ccc(-c4cccc5ccc6c7ccccc7ccc6c45)c4oc5ccccc5c34)n2)cc1 ; c1ccc(-c2nc(-c3ccccc3)nc(-c3cccc4oc5c(-c6cccc7ccc8c9ccccc9ccc8c67)cccc5c34)n2)cc1]

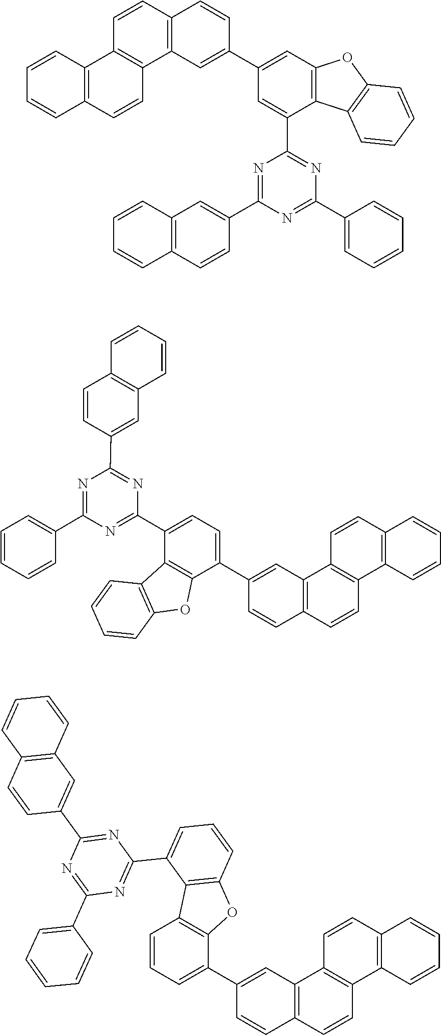 [SMILES: c1ccc(-c2nc(-c3ccc4ccccc4c3)nc(-c3cc(-c4ccc5ccc6c7ccccc7ccc6c5c4)cc4oc5ccccc5c34)n2)cc1 ; c1ccc(-c2nc(-c3ccc4ccccc4c3)nc(-c3ccc(-c4ccc5ccc6c7ccccc7ccc6c5c4)c4oc5ccccc5c34)n2)cc1 ; c1ccc(-c2nc(-c3ccc4ccccc4c3)nc(-c3cccc4oc5c(-c6ccc7ccc8c9ccccc9ccc8c7c6)cccc5c34)n2)cc1]

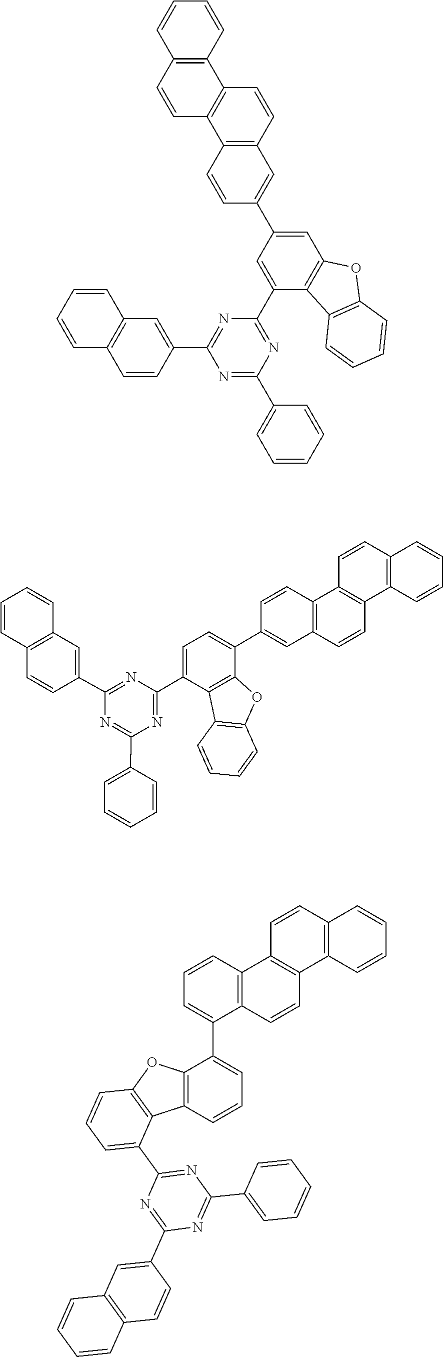 [SMILES: c1ccc(-c2nc(-c3ccc4ccccc4c3)nc(-c3cc(-c4ccc5c(ccc6c7ccccc7ccc56)c4)cc4oc5ccccc5c34)n2)cc1 ; c1ccc(-c2nc(-c3ccc4ccccc4c3)nc(-c3ccc(-c4ccc5c(ccc6c7ccccc7ccc56)c4)c4oc5ccccc5c34)n2)cc1 ; c1ccc(-c2nc(-c3ccc4ccccc4c3)nc(-c3cccc4oc5c(-c6cccc7c6ccc6c8ccccc8ccc76)cccc5c34)n2)cc1]

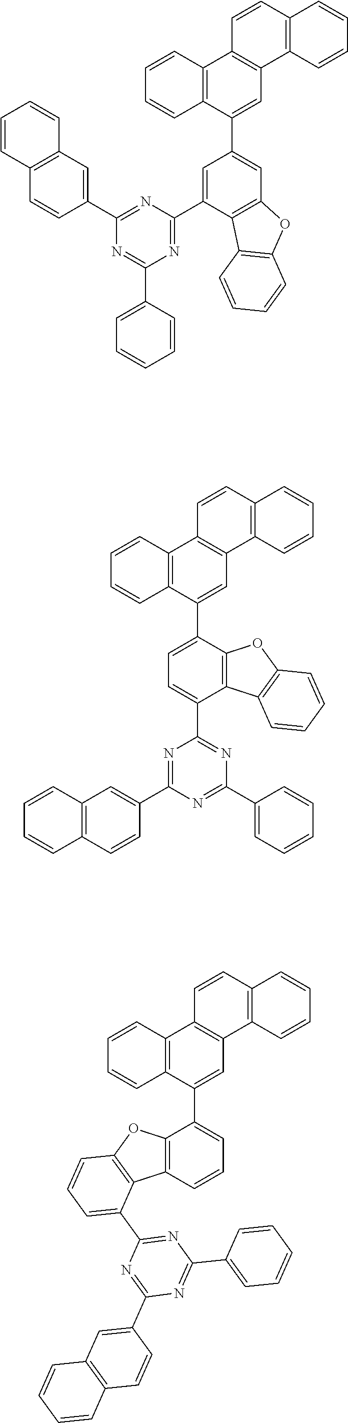 [SMILES: c1ccc(-c2nc(-c3ccc4ccccc4c3)nc(-c3cc(-c4cc5c6ccccc6ccc5c5ccccc45)cc4oc5ccccc5c34)n2)cc1 ; c1ccc(-c2nc(-c3ccc4ccccc4c3)nc(-c3ccc(-c4cc5c6ccccc6ccc5c5ccccc45)c4oc5ccccc5c34)n2)cc1 ; c1ccc(-c2nc(-c3ccc4ccccc4c3)nc(-c3cccc4oc5c(-c6cc7c8ccccc8ccc7c7ccccc67)cccc5c34)n2)cc1]

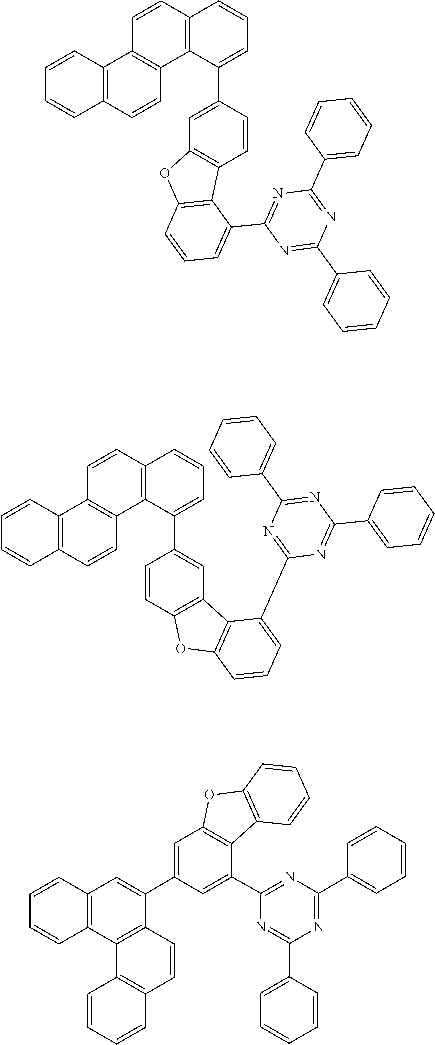 [SMILES: c1ccc(-c2nc(-c3ccccc3)nc(-c3cccc4oc5cc(-c6cccc7ccc8c9ccccc9ccc8c67)ccc5c34)n2)cc1 ; c1ccc(-c2nc(-c3ccccc3)nc(-c3cccc4oc5ccc(-c6cccc7ccc8c9ccccc9ccc8c67)cc5c34)n2)cc1 ; c1ccc(-c2nc(-c3ccccc3)nc(-c3cc(-c4cc5ccccc5c5c4ccc4ccccc45)cc4oc5ccccc5c34)n2)cc1]

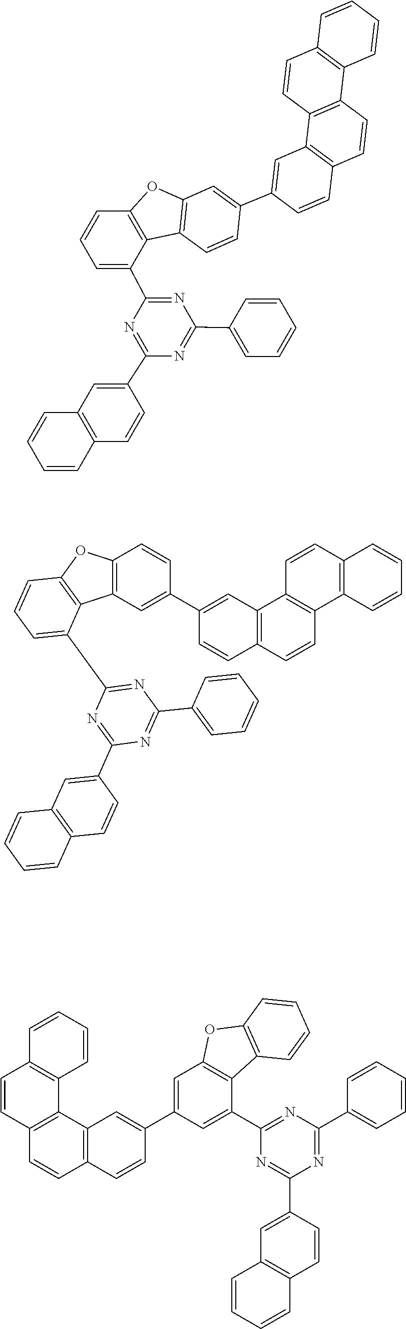 [SMILES: c1ccc(-c2nc(-c3ccc4ccccc4c3)nc(-c3cccc4oc5cc(-c6ccc7ccc8c9ccccc9ccc8c7c6)ccc5c34)n2)cc1 ; c1ccc(-c2nc(-c3ccc4ccccc4c3)nc(-c3cccc4oc5ccc(-c6ccc7ccc8c9ccccc9ccc8c7c6)cc5c34)n2)cc1 ; c1ccc(-c2nc(-c3ccc4ccccc4c3)nc(-c3cc(-c4ccc5ccc6ccc7ccccc7c6c5c4)cc4oc5ccccc5c34)n2)cc1]

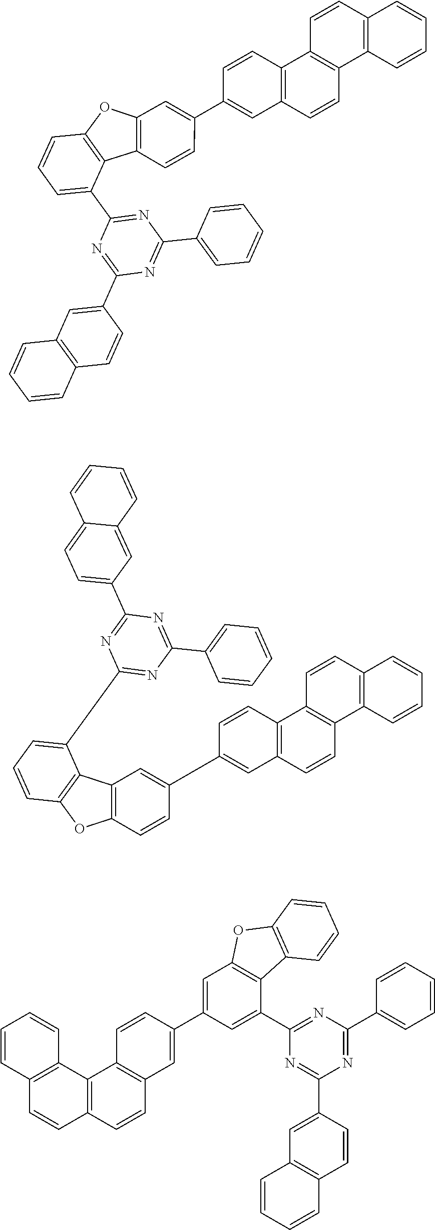 [SMILES: c1ccc(-c2nc(-c3ccc4ccccc4c3)nc(-c3cccc4oc5cc(-c6ccc7c(ccc8c9ccccc9ccc78)c6)ccc5c34)n2)cc1 ; c1ccc(-c2nc(-c3ccc4ccccc4c3)nc(-c3cccc4oc5ccc(-c6ccc7c(ccc8c9ccccc9ccc78)c6)cc5c34)n2)cc1 ; c1ccc(-c2nc(-c3ccc4ccccc4c3)nc(-c3cc(-c4ccc5c(ccc6ccc7ccccc7c65)c4)cc4oc5ccccc5c34)n2)cc1]

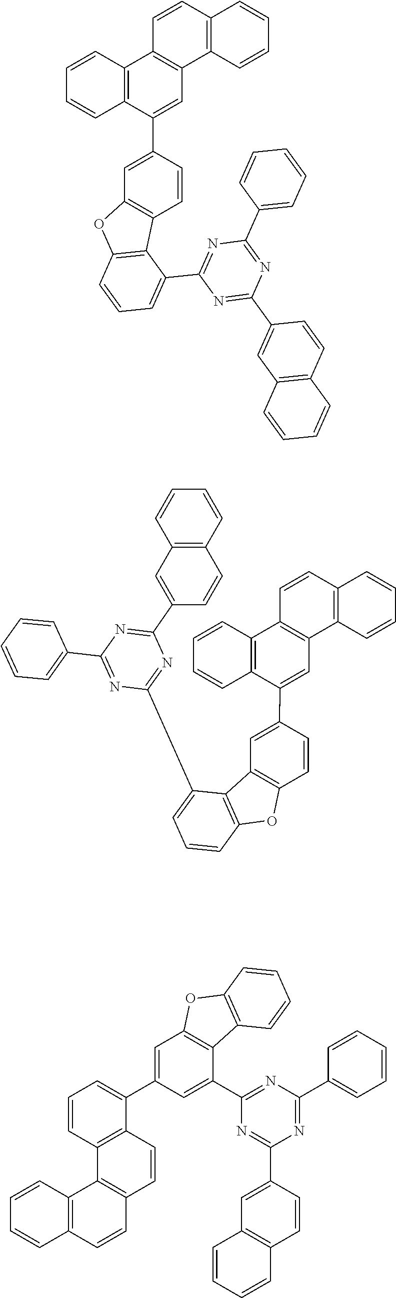 [SMILES: c1ccc(-c2nc(-c3ccc4ccccc4c3)nc(-c3cccc4oc5cc(-c6cc7c8ccccc8ccc7c7ccccc67)ccc5c34)n2)cc1 ; c1ccc(-c2nc(-c3ccc4ccccc4c3)nc(-c3cccc4oc5ccc(-c6cc7c8ccccc8ccc7c7ccccc67)cc5c34)n2)cc1 ; c1ccc(-c2nc(-c3ccc4ccccc4c3)nc(-c3cc(-c4cccc5c4ccc4ccc6ccccc6c45)cc4oc5ccccc5c34)n2)cc1]

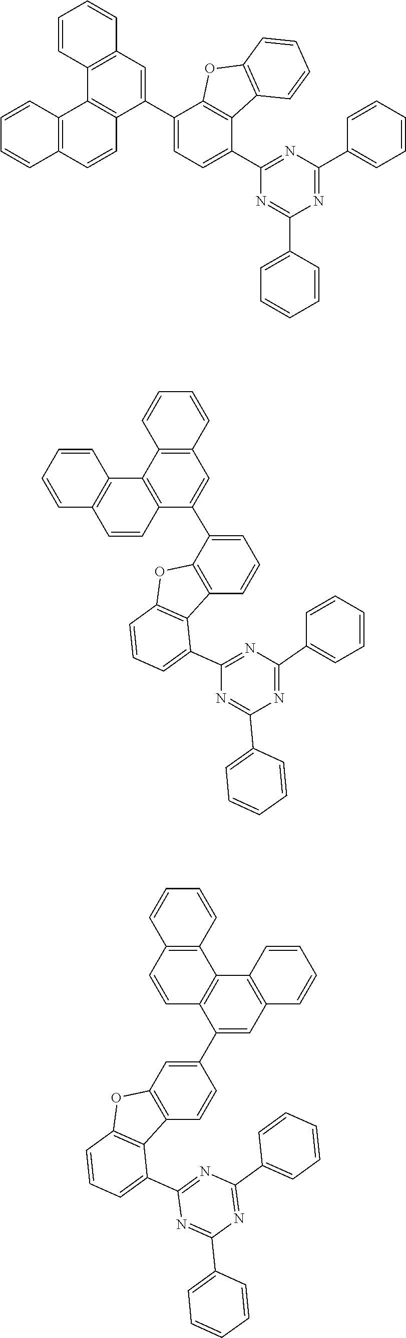 [SMILES: c1ccc(-c2nc(-c3ccccc3)nc(-c3ccc(-c4cc5ccccc5c5c4ccc4ccccc45)c4oc5ccccc5c34)n2)cc1 ; c1ccc(-c2nc(-c3ccccc3)nc(-c3cccc4oc5c(-c6cc7ccccc7c7c6ccc6ccccc67)cccc5c34)n2)cc1 ; c1ccc(-c2nc(-c3ccccc3)nc(-c3cccc4oc5cc(-c6cc7ccccc7c7c6ccc6ccccc67)ccc5c34)n2)cc1]

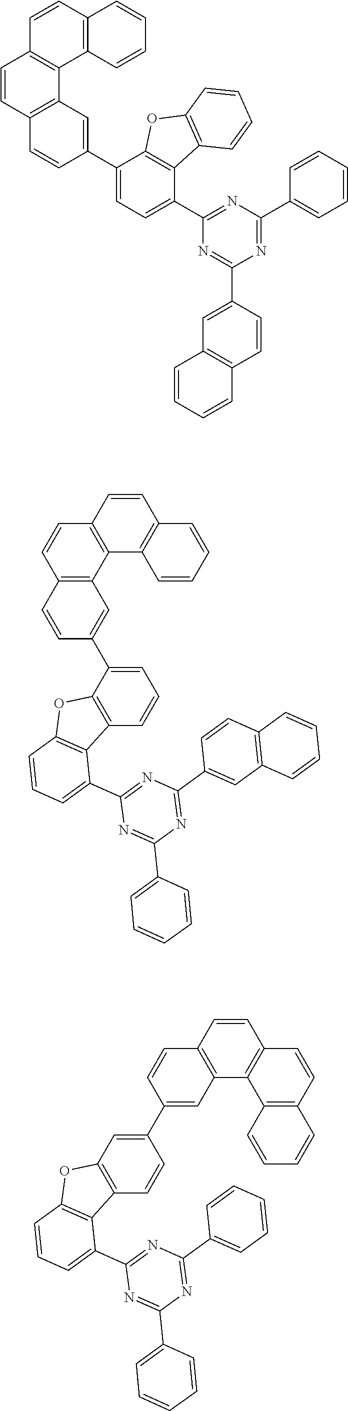 [SMILES: c1ccc(-c2nc(-c3ccc4ccccc4c3)nc(-c3ccc(-c4ccc5ccc6ccc7ccccc7c6c5c4)c4oc5ccccc5c34)n2)cc1 ; c1ccc(-c2nc(-c3ccc4ccccc4c3)nc(-c3cccc4oc5c(-c6ccc7ccc8ccc9ccccc9c8c7c6)cccc5c34)n2)cc1 ; c1ccc(-c2nc(-c3ccccc3)nc(-c3cccc4oc5cc(-c6ccc7ccc8ccc9ccccc9c8c7c6)ccc5c34)n2)cc1]

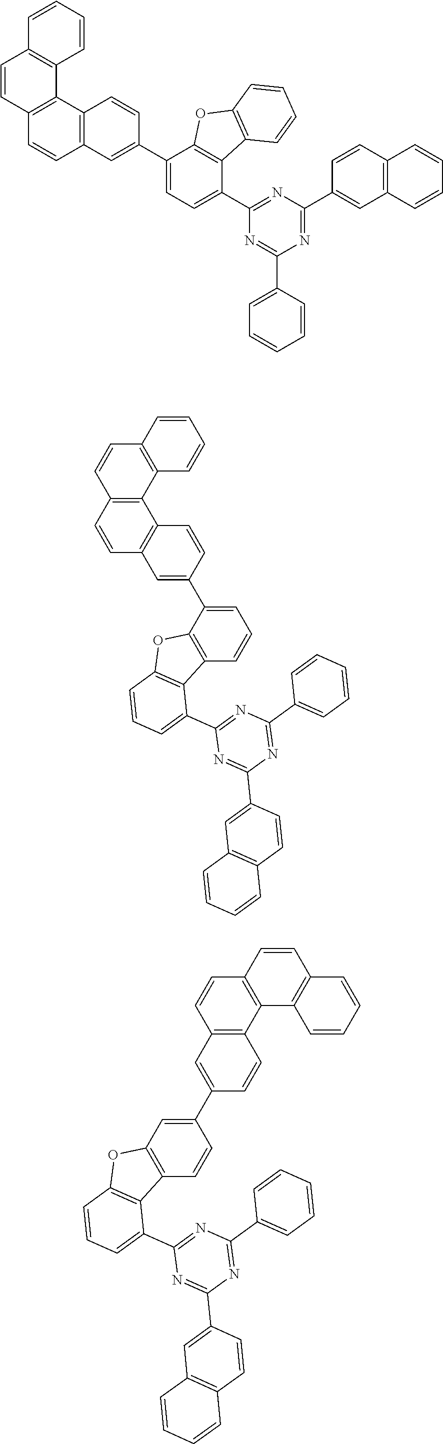

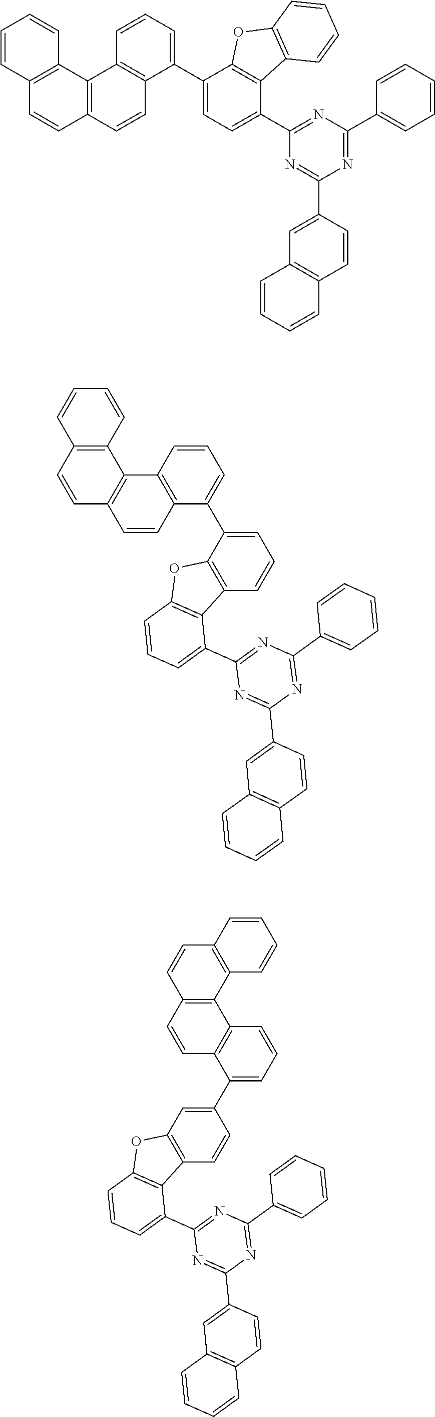 [SMILES: c1ccc(-c2nc(-c3ccc4ccccc4c3)nc(-c3ccc(-c4cccc5c4ccc4ccc6ccccc6c45)c4oc5ccccc5c34)n2)cc1 ; c1ccc(-c2nc(-c3ccc4ccccc4c3)nc(-c3cccc4oc5c(-c6cccc7c6ccc6ccc8ccccc8c67)cccc5c34)n2)cc1 ; c1ccc(-c2nc(-c3ccc4ccccc4c3)nc(-c3cccc4oc5cc(-c6cccc7c6ccc6ccc8ccccc8c67)ccc5c34)n2)cc1]

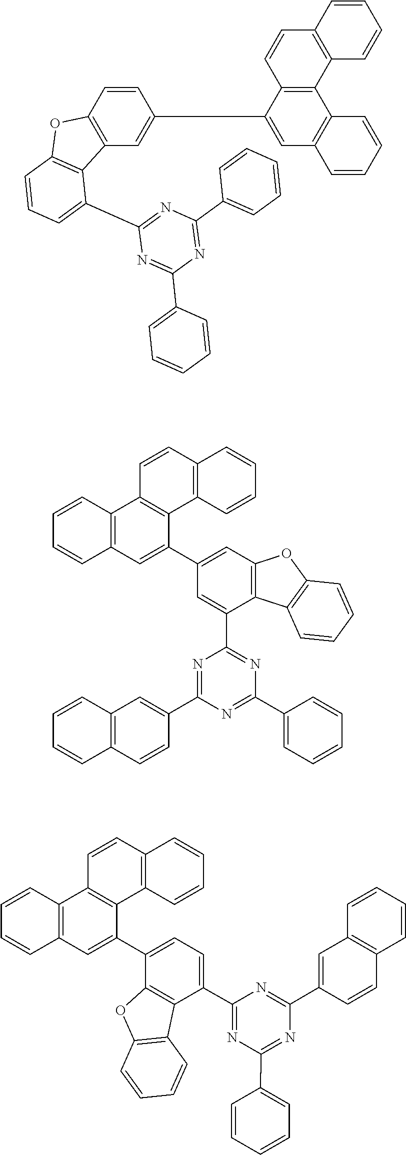 [SMILES: c1ccc(-c2nc(-c3ccccc3)nc(-c3cccc4oc5ccc(-c6cc7ccccc7c7c6ccc6ccccc67)cc5c34)n2)cc1 ; c1ccc(-c2nc(-c3ccc4ccccc4c3)nc(-c3cc(-c4cc5ccccc5c5ccc6ccccc6c45)cc4oc5ccccc5c34)n2)cc1 ; c1ccc(-c2nc(-c3ccc4ccccc4c3)nc(-c3ccc(-c4cc5ccccc5c5ccc6ccccc6c45)c4oc5ccccc5c34)n2)cc1]

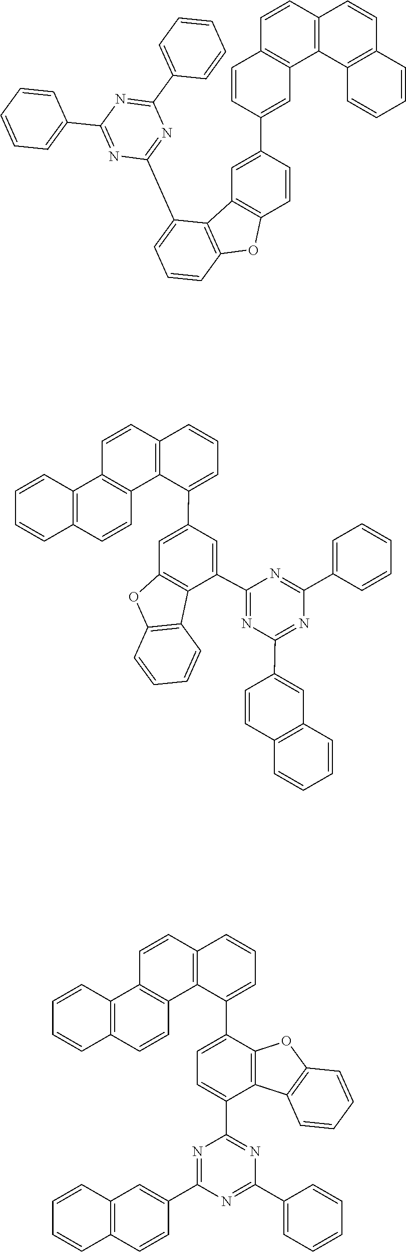 [SMILES: c1ccc(-c2nc(-c3ccccc3)nc(-c3cccc4oc5ccc(-c6ccc7ccc8ccc9ccccc9c8c7c6)cc5c34)n2)cc1 ; c1ccc(-c2nc(-c3ccc4ccccc4c3)nc(-c3cc(-c4cccc5ccc6c7ccccc7ccc6c45)cc4oc5ccccc5c34)n2)cc1 ; c1ccc(-c2nc(-c3ccc4ccccc4c3)nc(-c3ccc(-c4cccc5ccc6c7ccccc7ccc6c45)c4oc5ccccc5c34)n2)cc1]

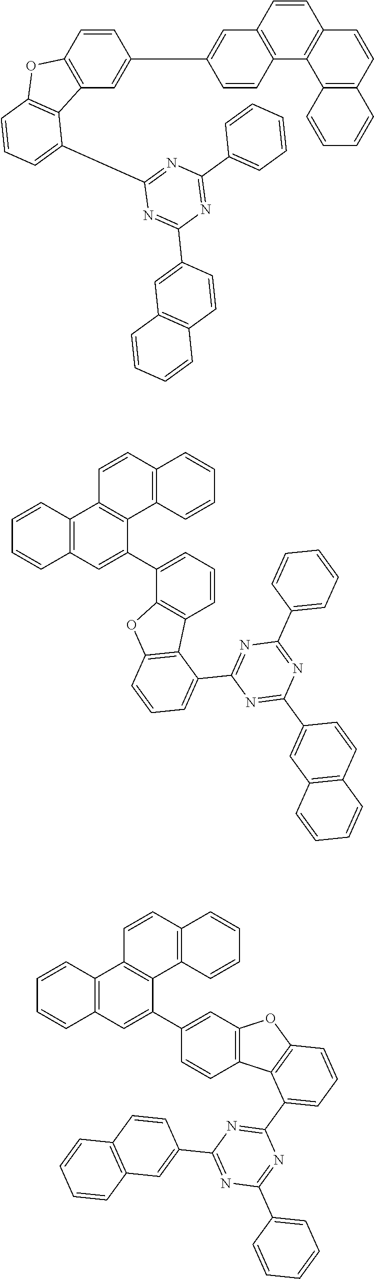 [SMILES: c1ccc(-c2nc(-c3ccc4ccccc4c3)nc(-c3cccc4oc5ccc(-c6ccc7c(ccc8ccc9ccccc9c87)c6)cc5c34)n2)cc1 ; c1ccc(-c2nc(-c3ccc4ccccc4c3)nc(-c3cccc4oc5c(-c6cc7ccccc7c7ccc8ccccc8c67)cccc5c34)n2)cc1 ; c1ccc(-c2nc(-c3ccc4ccccc4c3)nc(-c3cccc4oc5cc(-c6cc7ccccc7c7ccc8ccccc8c67)ccc5c34)n2)cc1]

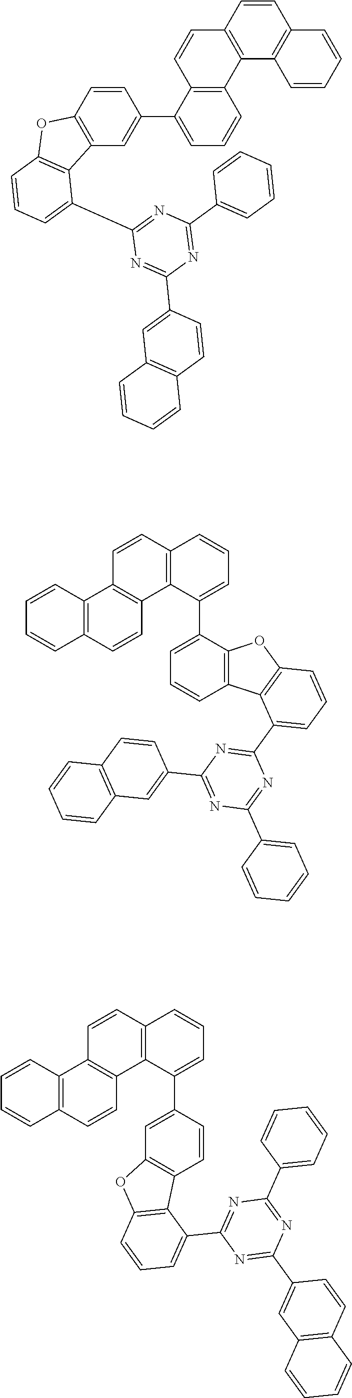 [SMILES: c1ccc(-c2nc(-c3ccc4ccccc4c3)nc(-c3cccc4oc5ccc(-c6cccc7c6ccc6ccc8ccccc8c67)cc5c34)n2)cc1 ; c1ccc(-c2nc(-c3ccc4ccccc4c3)nc(-c3cccc4oc5c(-c6cccc7ccc8c9ccccc9ccc8c67)cccc5c34)n2)cc1 ; c1ccc(-c2nc(-c3ccc4ccccc4c3)nc(-c3cccc4oc5cc(-c6cccc7ccc8c9ccccc9ccc8c67)ccc5c34)n2)cc1]

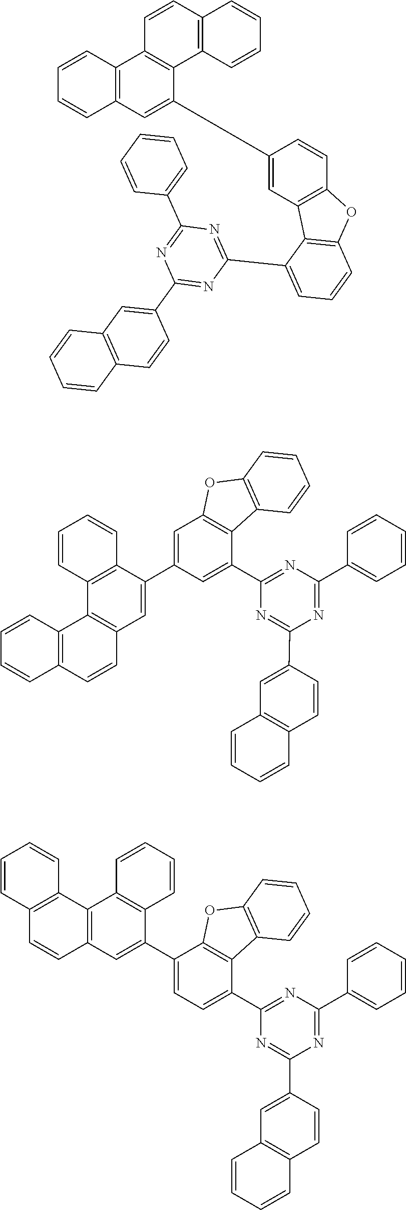 [SMILES: c1ccc(-c2nc(-c3ccc4ccccc4c3)nc(-c3cccc4oc5ccc(-c6cc7ccccc7c7ccc8ccccc8c67)cc5c34)n2)cc1 ; c1ccc(-c2nc(-c3ccc4ccccc4c3)nc(-c3cc(-c4cc5ccc6ccccc6c5c5ccccc45)cc4oc5ccccc5c34)n2)cc1 ; c1ccc(-c2nc(-c3ccc4ccccc4c3)nc(-c3ccc(-c4cc5ccc6ccccc6c5c5ccccc45)c4oc5ccccc5c34)n2)cc1]

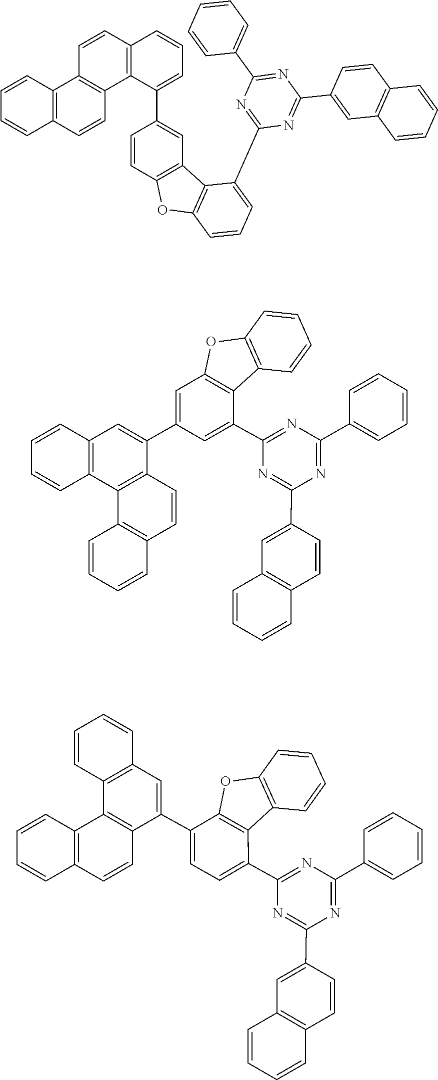 [SMILES: c1ccc(-c2nc(-c3ccc4ccccc4c3)nc(-c3cccc4oc5ccc(-c6cccc7ccc8c9ccccc9ccc8c67)cc5c34)n2)cc1 ; c1ccc(-c2nc(-c3ccc4ccccc4c3)nc(-c3cc(-c4cc5ccccc5c5c4ccc4ccccc45)cc4oc5ccccc5c34)n2)cc1 ; c1ccc(-c2nc(-c3ccc4ccccc4c3)nc(-c3ccc(-c4cc5ccccc5c5c4ccc4ccccc45)c4oc5ccccc5c34)n2)cc1]

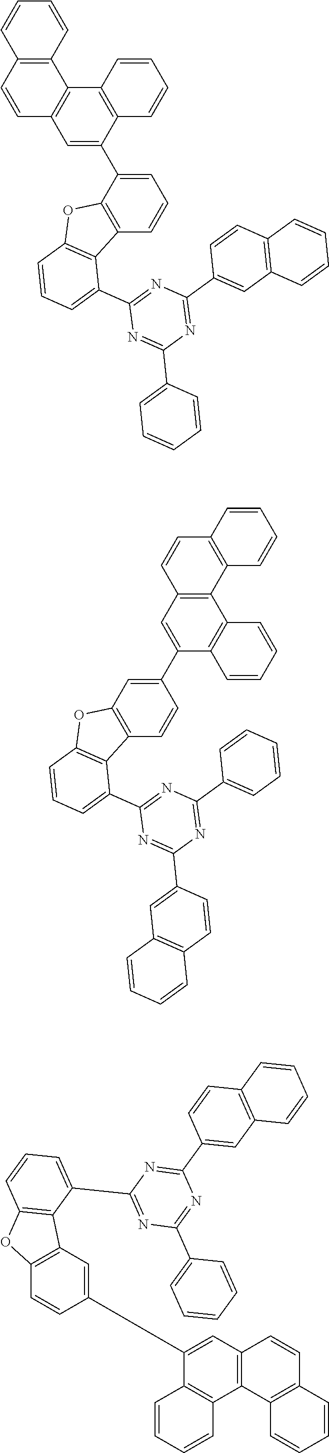 [SMILES: c1ccc(-c2nc(-c3ccc4ccccc4c3)nc(-c3cccc4oc5c(-c6cc7ccc8ccccc8c7c7ccccc67)cccc5c34)n2)cc1 ; c1ccc(-c2nc(-c3ccc4ccccc4c3)nc(-c3cccc4oc5cc(-c6cc7ccc8ccccc8c7c7ccccc67)ccc5c34)n2)cc1 ; c1ccc(-c2nc(-c3ccc4ccccc4c3)nc(-c3cccc4oc5ccc(-c6cc7ccc8ccccc8c7c7ccccc67)cc5c34)n2)cc1]

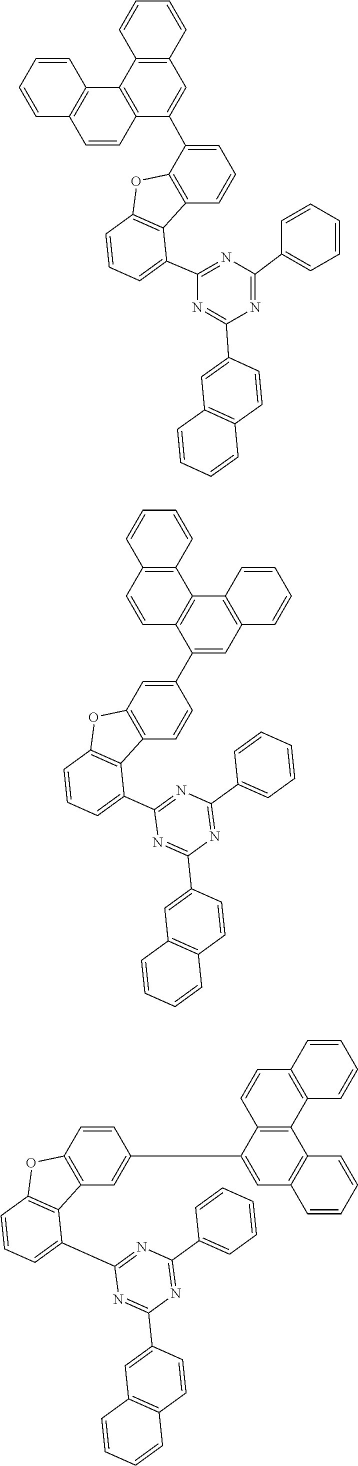 [SMILES: c1ccc(-c2nc(-c3ccc4ccccc4c3)nc(-c3cccc4oc5c(-c6cc7ccccc7c7c6ccc6ccccc67)cccc5c34)n2)cc1 ; c1ccc(-c2nc(-c3ccc4ccccc4c3)nc(-c3cccc4oc5cc(-c6cc7ccccc7c7c6ccc6ccccc67)ccc5c34)n2)cc1 ; c1ccc(-c2nc(-c3ccc4ccccc4c3)nc(-c3cccc4oc5ccc(-c6cc7ccccc7c7c6ccc6ccccc67)cc5c34)n2)cc1]

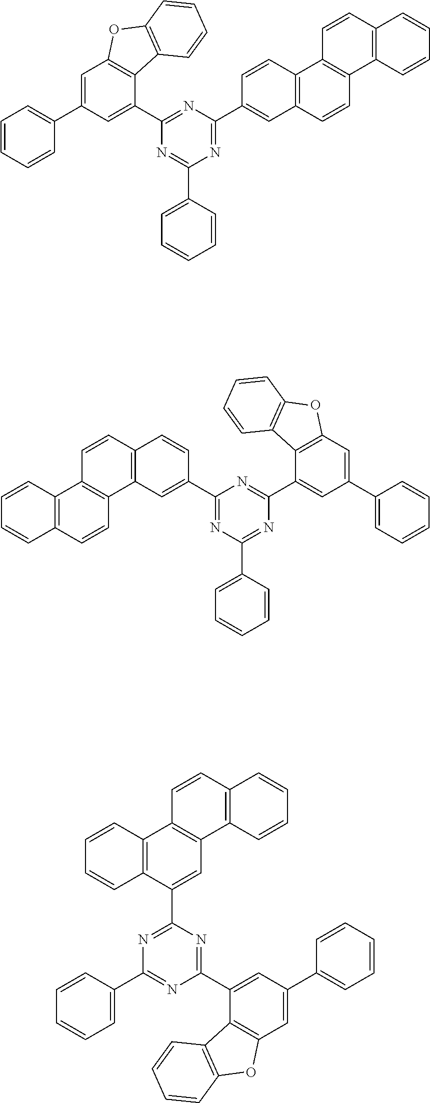 [SMILES: c1ccc(-c2cc(-c3nc(-c4ccccc4)nc(-c4ccc5c(ccc6c7ccccc7ccc56)c4)n3)c3c(c2)oc2ccccc23)cc1 ; c1ccc(-c2cc(-c3nc(-c4ccccc4)nc(-c4ccc5ccc6c7ccccc7ccc6c5c4)n3)c3c(c2)oc2ccccc23)cc1 ; c1ccc(-c2cc(-c3nc(-c4ccccc4)nc(-c4cc5c6ccccc6ccc5c5ccccc45)n3)c3c(c2)oc2ccccc23)cc1]

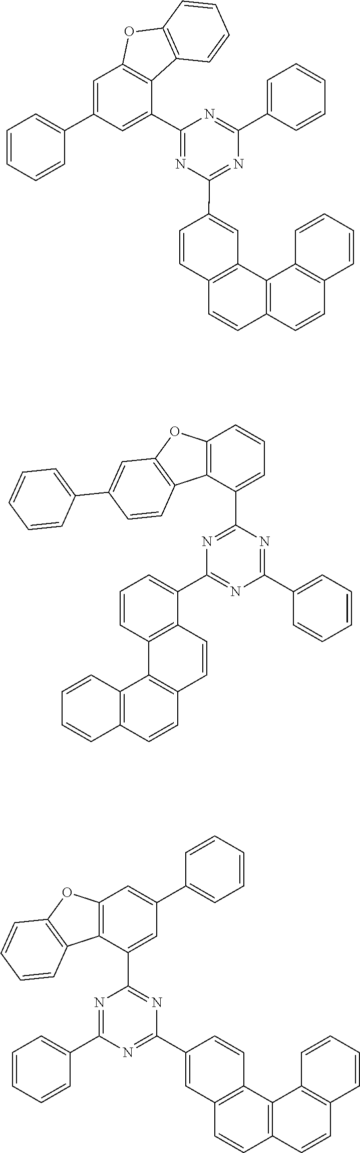 [SMILES: c1ccc(-c2cc(-c3nc(-c4ccccc4)nc(-c4ccc5ccc6ccc7ccccc7c6c5c4)n3)c3c(c2)oc2ccccc23)cc1 ; c1ccc(-c2ccc3c(c2)oc2cccc(-c4nc(-c5ccccc5)nc(-c5cccc6c5ccc5ccc7ccccc7c56)n4)c23)cc1 ; c1ccc(-c2cc(-c3nc(-c4ccccc4)nc(-c4ccc5c(ccc6ccc7ccccc7c65)c4)n3)c3c(c2)oc2ccccc23)cc1]

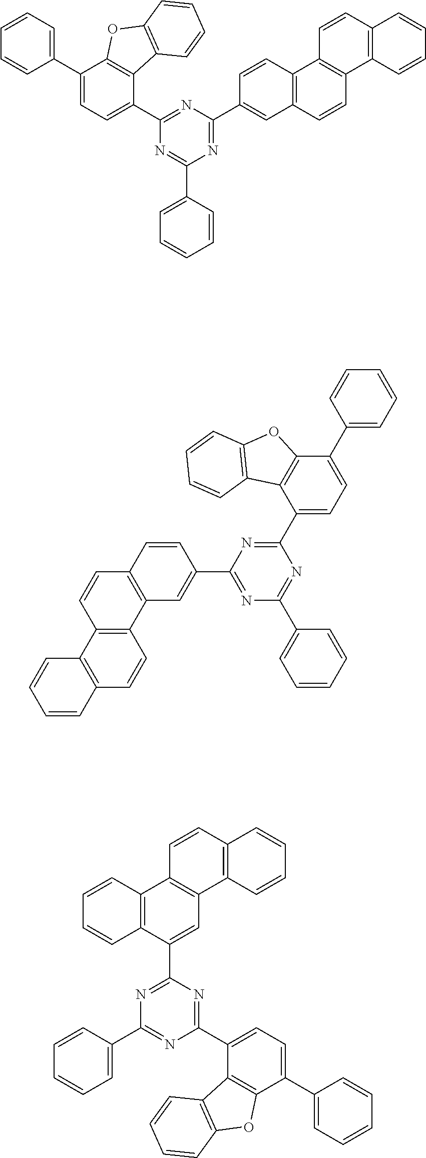 [SMILES: c1ccc(-c2nc(-c3ccc4c(ccc5c6ccccc6ccc45)c3)nc(-c3ccc(-c4ccccc4)c4oc5ccccc5c34)n2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2nc(-c3cc4c5ccccc5ccc4c4ccccc34)nc(-c3ccc(-c4ccccc4)c4oc5ccccc5c34)n2)cc1]

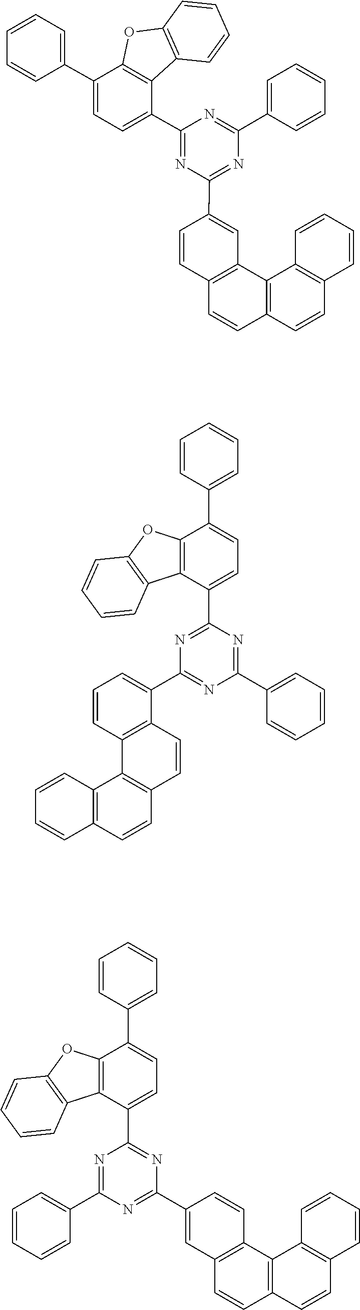 [SMILES: c1ccc(-c2nc(-c3ccc4ccc5ccc6ccccc6c5c4c3)nc(-c3ccc(-c4ccccc4)c4oc5ccccc5c34)n2)cc1 ; c1ccc(-c2nc(-c3cccc4c3ccc3ccc5ccccc5c34)nc(-c3ccc(-c4ccccc4)c4oc5ccccc5c34)n2)cc1 ; c1ccc(-c2nc(-c3ccc4c(ccc5ccc6ccccc6c54)c3)nc(-c3ccc(-c4ccccc4)c4oc5ccccc5c34)n2)cc1]

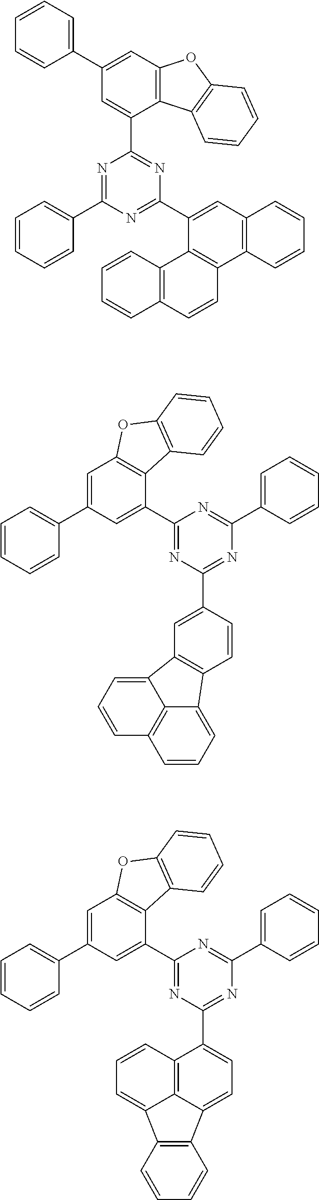 [SMILES: c1ccc(-c2cc(-c3nc(-c4ccccc4)nc(-c4cc5ccccc5c5ccc6ccccc6c45)n3)c3c(c2)oc2ccccc23)cc1 ; c1ccc(-c2cc(-c3nc(-c4ccccc4)nc(-c4ccc5c(c4)-c4cccc6cccc-5c46)n3)c3c(c2)oc2ccccc23)cc1 ; c1ccc(-c2cc(-c3nc(-c4ccccc4)nc(-c4ccc5c6c(cccc46)-c4ccccc4-5)n3)c3c(c2)oc2ccccc23)cc1]

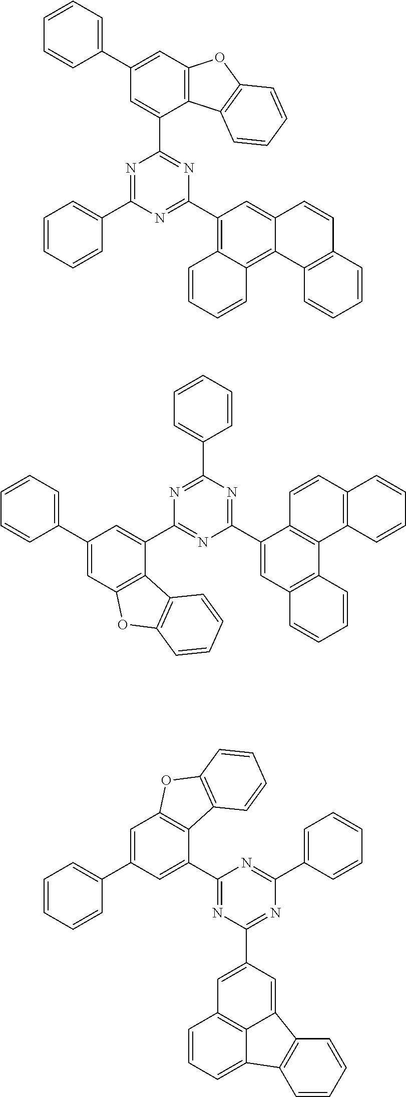 [SMILES: c1ccc(-c2cc(-c3nc(-c4ccccc4)nc(-c4cc5ccc6ccccc6c5c5ccccc45)n3)c3c(c2)oc2ccccc23)cc1 ; c1ccc(-c2cc(-c3nc(-c4ccccc4)nc(-c4cc5ccccc5c5c4ccc4ccccc45)n3)c3c(c2)oc2ccccc23)cc1 ; c1ccc(-c2cc(-c3nc(-c4ccccc4)nc(-c4cc5c6c(cccc6c4)-c4ccccc4-5)n3)c3c(c2)oc2ccccc23)cc1]

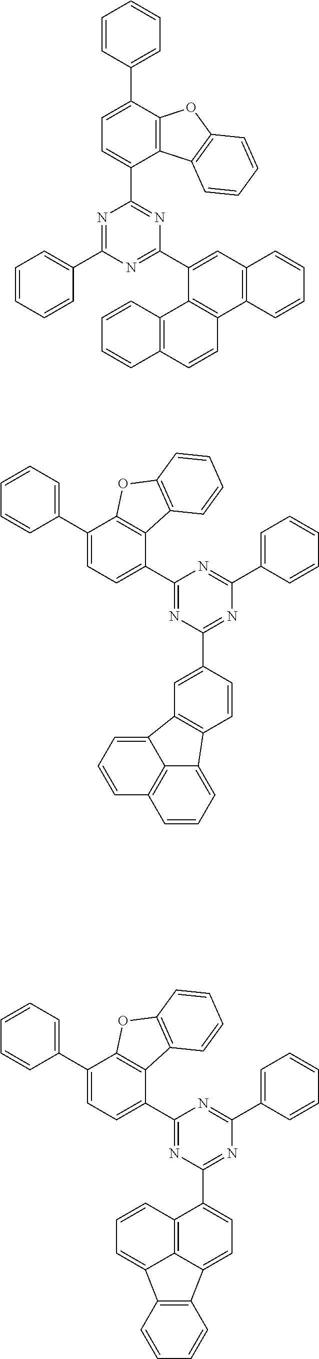 [SMILES: c1ccc(-c2nc(-c3ccc(-c4ccccc4)c4oc5ccccc5c34)nc(-c3cc4ccccc4c4ccc5ccccc5c34)n2)cc1 ; c1ccc(-c2nc(-c3ccc4c(c3)-c3cccc5cccc-4c35)nc(-c3ccc(-c4ccccc4)c4oc5ccccc5c34)n2)cc1 ; c1ccc(-c2nc(-c3ccc4c5c(cccc35)-c3ccccc3-4)nc(-c3ccc(-c4ccccc4)c4oc5ccccc5c34)n2)cc1]

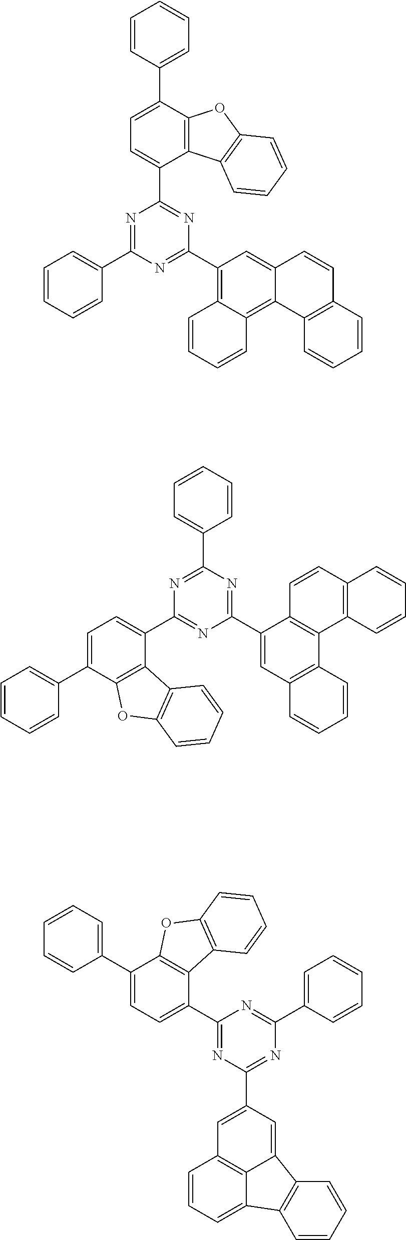 [SMILES: c1ccc(-c2nc(-c3cc4ccc5ccccc5c4c4ccccc34)nc(-c3ccc(-c4ccccc4)c4oc5ccccc5c34)n2)cc1 ; c1ccc(-c2nc(-c3cc4ccccc4c4c3ccc3ccccc34)nc(-c3ccc(-c4ccccc4)c4oc5ccccc5c34)n2)cc1 ; c1ccc(-c2nc(-c3cc4c5c(cccc5c3)-c3ccccc3-4)nc(-c3ccc(-c4ccccc4)c4oc5ccccc5c34)n2)cc1]

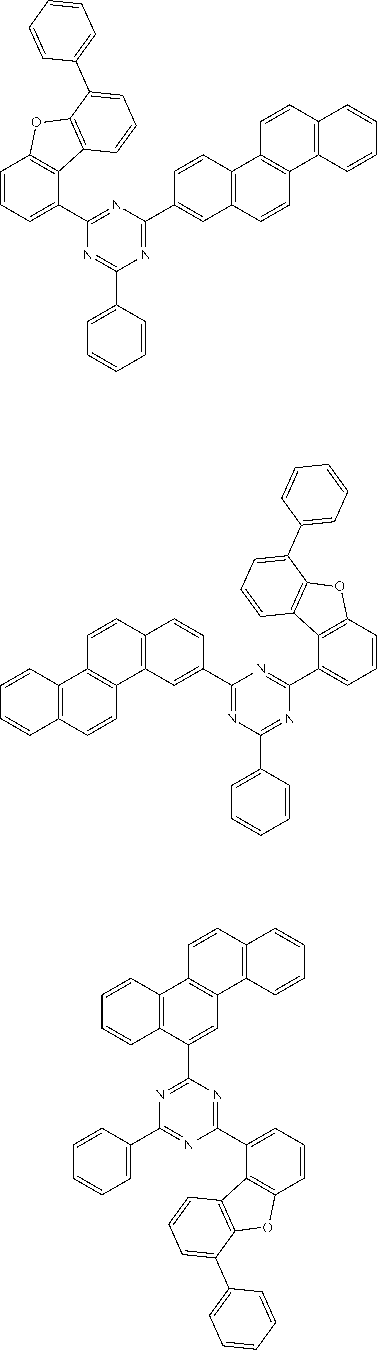 [SMILES: c1ccc(-c2nc(-c3ccc4c(ccc5c6ccccc6ccc45)c3)nc(-c3cccc4oc5c(-c6ccccc6)cccc5c34)n2)cc1 ; c1ccc(-c2nc(-c3ccc4ccc5c6ccccc6ccc5c4c3)nc(-c3cccc4oc5c(-c6ccccc6)cccc5c34)n2)cc1 ; c1ccc(-c2nc(-c3cc4c5ccccc5ccc4c4ccccc34)nc(-c3cccc4oc5c(-c6ccccc6)cccc5c34)n2)cc1]

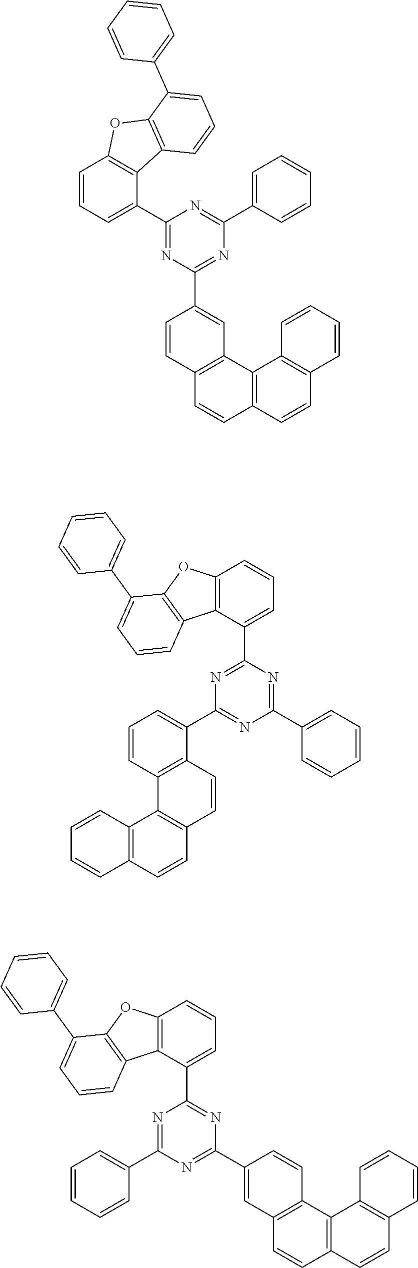 [SMILES: c1ccc(-c2nc(-c3ccc4ccc5ccc6ccccc6c5c4c3)nc(-c3cccc4oc5c(-c6ccccc6)cccc5c34)n2)cc1 ; c1ccc(-c2nc(-c3cccc4c3ccc3ccc5ccccc5c34)nc(-c3cccc4oc5c(-c6ccccc6)cccc5c34)n2)cc1 ; c1ccc(-c2nc(-c3ccc4c(ccc5ccc6ccccc6c54)c3)nc(-c3cccc4oc5c(-c6ccccc6)cccc5c34)n2)cc1]

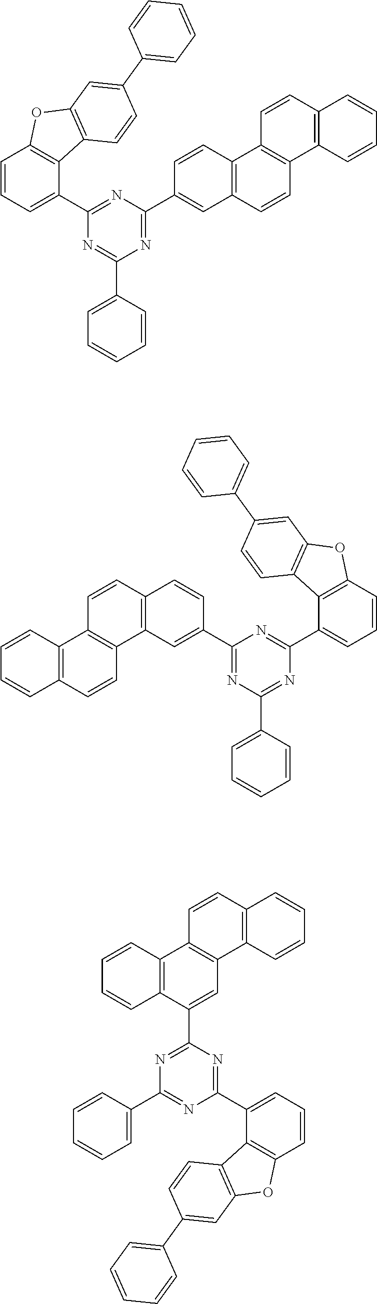 [SMILES: c1ccc(-c2ccc3c(c2)oc2cccc(-c4nc(-c5ccccc5)nc(-c5ccc6c(ccc7c8ccccc8ccc67)c5)n4)c23)cc1 ; c1ccc(-c2ccc3c(c2)oc2cccc(-c4nc(-c5ccccc5)nc(-c5ccc6ccc7c8ccccc8ccc7c6c5)n4)c23)cc1 ; c1ccc(-c2ccc3c(c2)oc2cccc(-c4nc(-c5ccccc5)nc(-c5cc6c7ccccc7ccc6c6ccccc56)n4)c23)cc1]

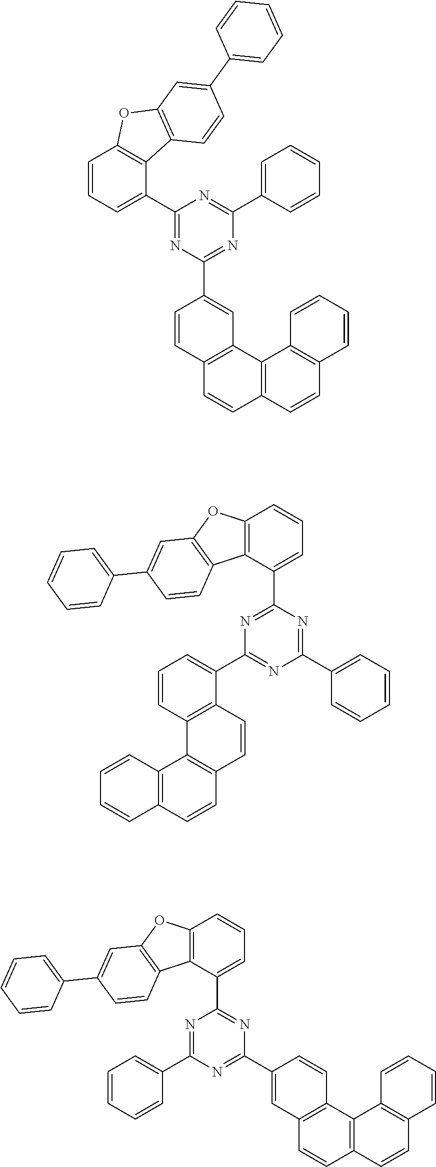 [SMILES: c1ccc(-c2ccc3c(c2)oc2cccc(-c4nc(-c5ccccc5)nc(-c5ccc6ccc7ccc8ccccc8c7c6c5)n4)c23)cc1 ; c1ccc(-c2ccc3c(c2)oc2cccc(-c4nc(-c5ccccc5)nc(-c5cccc6c5ccc5ccc7ccccc7c56)n4)c23)cc1 ; c1ccc(-c2ccc3c(c2)oc2cccc(-c4nc(-c5ccccc5)nc(-c5ccc6c(ccc7ccc8ccccc8c76)c5)n4)c23)cc1]

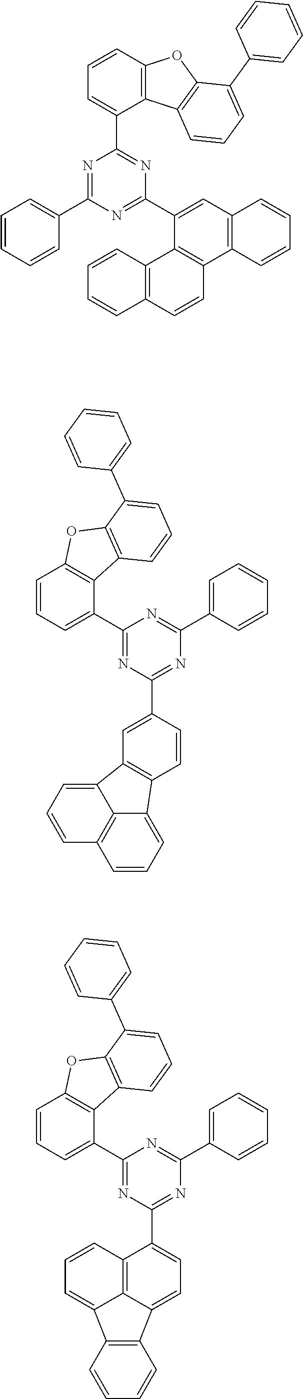 [SMILES: c1ccc(-c2nc(-c3cccc4oc5c(-c6ccccc6)cccc5c34)nc(-c3cc4ccccc4c4ccc5ccccc5c34)n2)cc1 ; c1ccc(-c2nc(-c3ccc4c(c3)-c3cccc5cccc-4c35)nc(-c3cccc4oc5c(-c6ccccc6)cccc5c34)n2)cc1 ; c1ccc(-c2nc(-c3ccc4c5c(cccc35)-c3ccccc3-4)nc(-c3cccc4oc5c(-c6ccccc6)cccc5c34)n2)cc1]

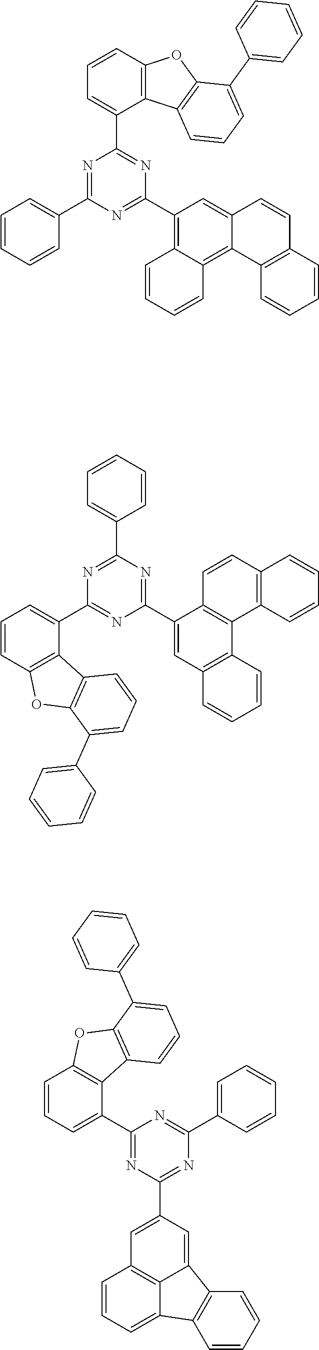 [SMILES: c1ccc(-c2nc(-c3cc4ccc5ccccc5c4c4ccccc34)nc(-c3cccc4oc5c(-c6ccccc6)cccc5c34)n2)cc1 ; c1ccc(-c2nc(-c3cc4ccccc4c4c3ccc3ccccc34)nc(-c3cccc4oc5c(-c6ccccc6)cccc5c34)n2)cc1 ; c1ccc(-c2nc(-c3cc4c5c(cccc5c3)-c3ccccc3-4)nc(-c3cccc4oc5c(-c6ccccc6)cccc5c34)n2)cc1]

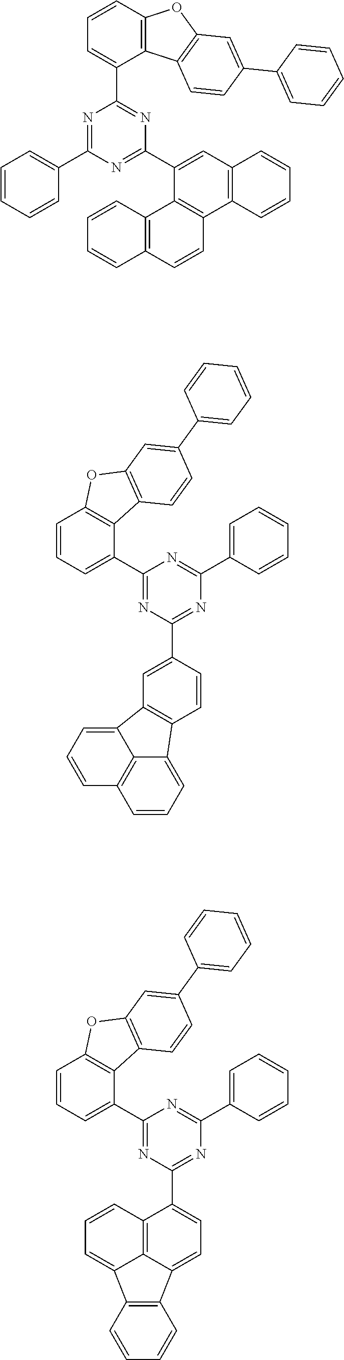 [SMILES: c1ccc(-c2ccc3c(c2)oc2cccc(-c4nc(-c5ccccc5)nc(-c5cc6ccccc6c6ccc7ccccc7c56)n4)c23)cc1 ; c1ccc(-c2ccc3c(c2)oc2cccc(-c4nc(-c5ccccc5)nc(-c5ccc6c(c5)-c5cccc7cccc-6c57)n4)c23)cc1 ; c1ccc(-c2ccc3c(c2)oc2cccc(-c4nc(-c5ccccc5)nc(-c5ccc6c7c(cccc57)-c5ccccc5-6)n4)c23)cc1]

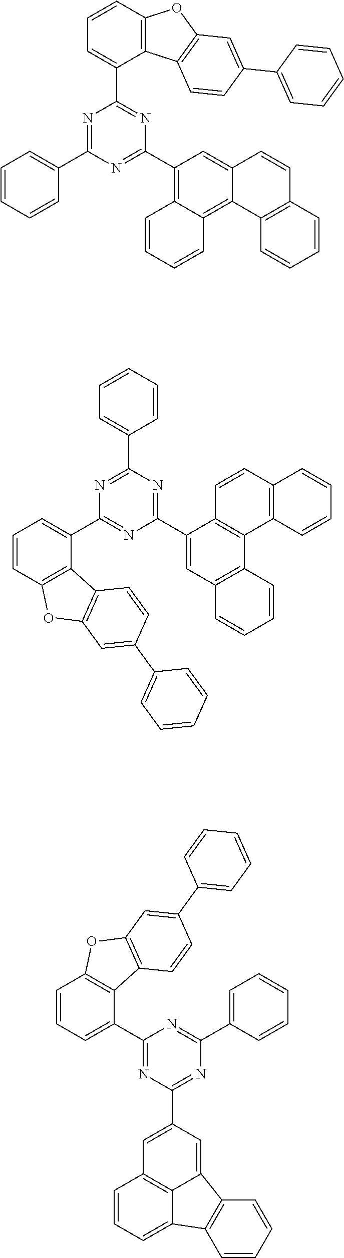 [SMILES: c1ccc(-c2ccc3c(c2)oc2cccc(-c4nc(-c5ccccc5)nc(-c5cc6ccc7ccccc7c6c6ccccc56)n4)c23)cc1 ; c1ccc(-c2ccc3c(c2)oc2cccc(-c4nc(-c5ccccc5)nc(-c5cc6ccccc6c6c5ccc5ccccc56)n4)c23)cc1 ; c1ccc(-c2ccc3c(c2)oc2cccc(-c4nc(-c5ccccc5)nc(-c5cc6c7c(cccc7c5)-c5ccccc5-6)n4)c23)cc1]

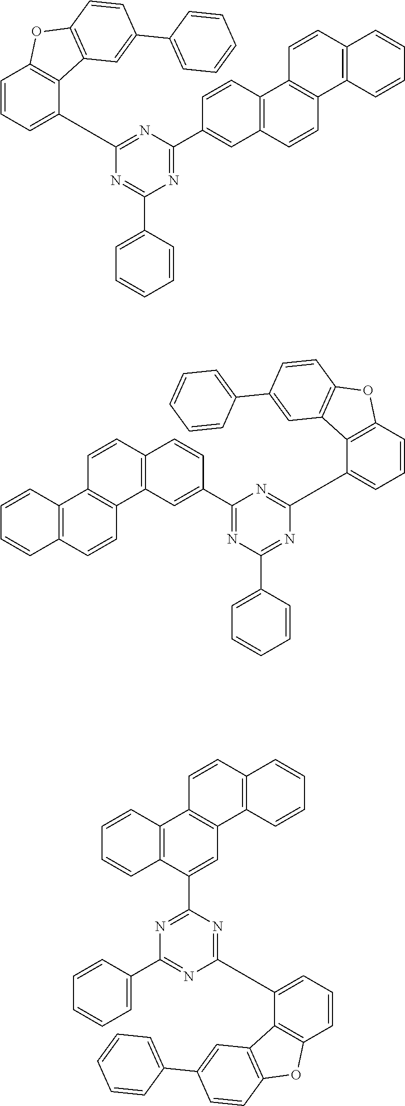 [SMILES: c1ccc(-c2ccc3oc4cccc(-c5nc(-c6ccccc6)nc(-c6ccc7c(ccc8c9ccccc9ccc78)c6)n5)c4c3c2)cc1 ; c1ccc(-c2ccc3oc4cccc(-c5nc(-c6ccccc6)nc(-c6ccc7ccc8c9ccccc9ccc8c7c6)n5)c4c3c2)cc1 ; c1ccc(-c2ccc3oc4cccc(-c5nc(-c6ccccc6)nc(-c6cc7c8ccccc8ccc7c7ccccc67)n5)c4c3c2)cc1]

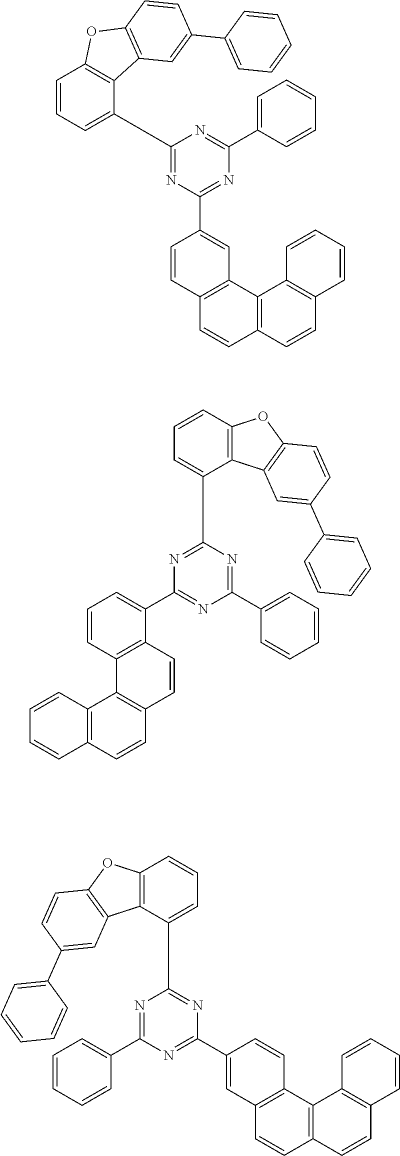 [SMILES: c1ccc(-c2ccc3oc4cccc(-c5nc(-c6ccccc6)nc(-c6ccc7ccc8ccc9ccccc9c8c7c6)n5)c4c3c2)cc1 ; c1ccc(-c2ccc3oc4cccc(-c5nc(-c6ccccc6)nc(-c6cccc7c6ccc6ccc8ccccc8c67)n5)c4c3c2)cc1 ; c1ccc(-c2ccc3oc4cccc(-c5nc(-c6ccccc6)nc(-c6ccc7c(ccc8ccc9ccccc9c87)c6)n5)c4c3c2)cc1]

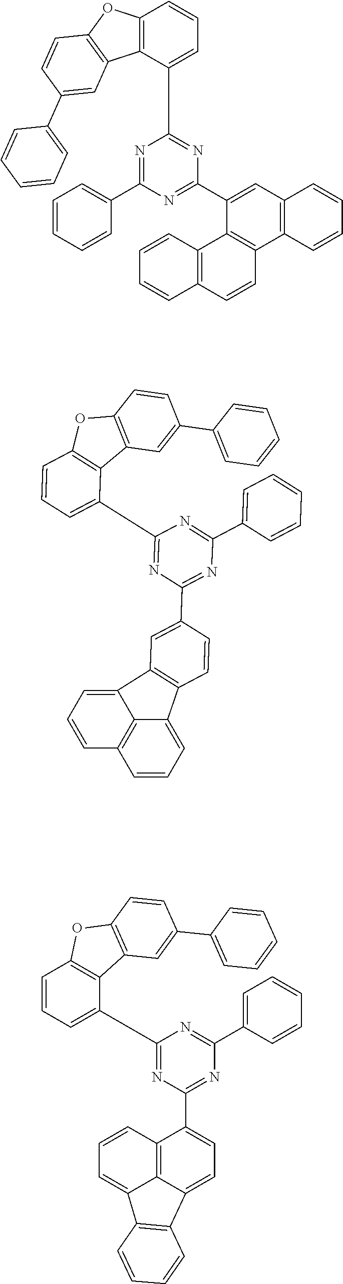 [SMILES: c1ccc(-c2ccc3oc4cccc(-c5nc(-c6ccccc6)nc(-c6cc7ccccc7c7ccc8ccccc8c67)n5)c4c3c2)cc1 ; c1ccc(-c2ccc3oc4cccc(-c5nc(-c6ccccc6)nc(-c6ccc7c(c6)-c6cccc8cccc-7c68)n5)c4c3c2)cc1 ; c1ccc(-c2ccc3oc4cccc(-c5nc(-c6ccccc6)nc(-c6ccc7c8c(cccc68)-c6ccccc6-7)n5)c4c3c2)cc1]

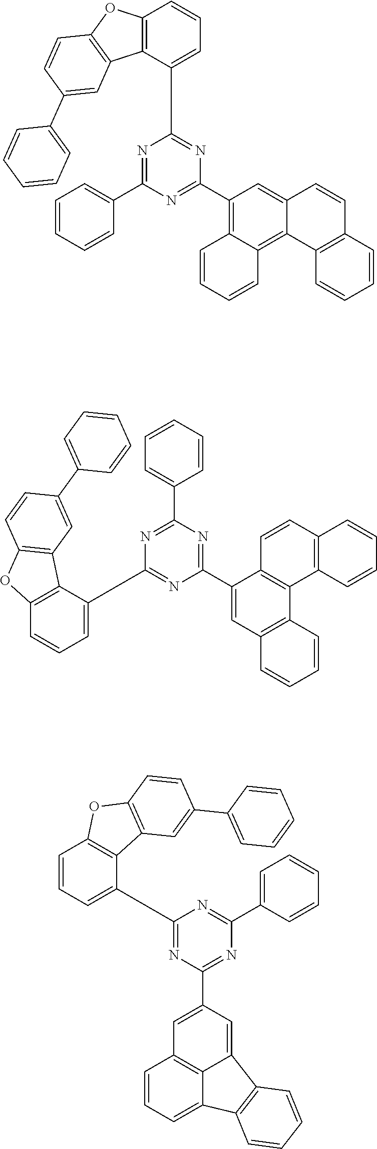 [SMILES: c1ccc(-c2ccc3oc4cccc(-c5nc(-c6ccccc6)nc(-c6cc7ccc8ccccc8c7c7ccccc67)n5)c4c3c2)cc1 ; c1ccc(-c2ccc3oc4cccc(-c5nc(-c6ccccc6)nc(-c6cc7ccccc7c7c6ccc6ccccc67)n5)c4c3c2)cc1 ; c1ccc(-c2ccc3oc4cccc(-c5nc(-c6ccccc6)nc(-c6cc7c8c(cccc8c6)-c6ccccc6-7)n5)c4c3c2)cc1]

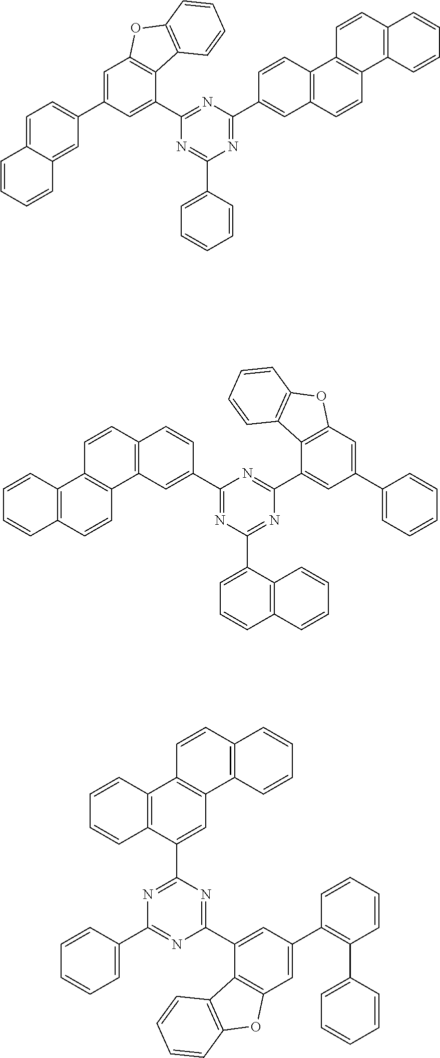 [SMILES: c1ccc(-c2nc(-c3ccc4c(ccc5c6ccccc6ccc45)c3)nc(-c3cc(-c4ccc5ccccc5c4)cc4oc5ccccc5c34)n2)cc1 ; c1ccc(-c2cc(-c3nc(-c4ccc5ccc6c7ccccc7ccc6c5c4)nc(-c4cccc5ccccc45)n3)c3c(c2)oc2ccccc23)cc1 ; c1ccc(-c2nc(-c3cc4c5ccccc5ccc4c4ccccc34)nc(-c3cc(-c4ccccc4-c4ccccc4)cc4oc5ccccc5c34)n2)cc1]

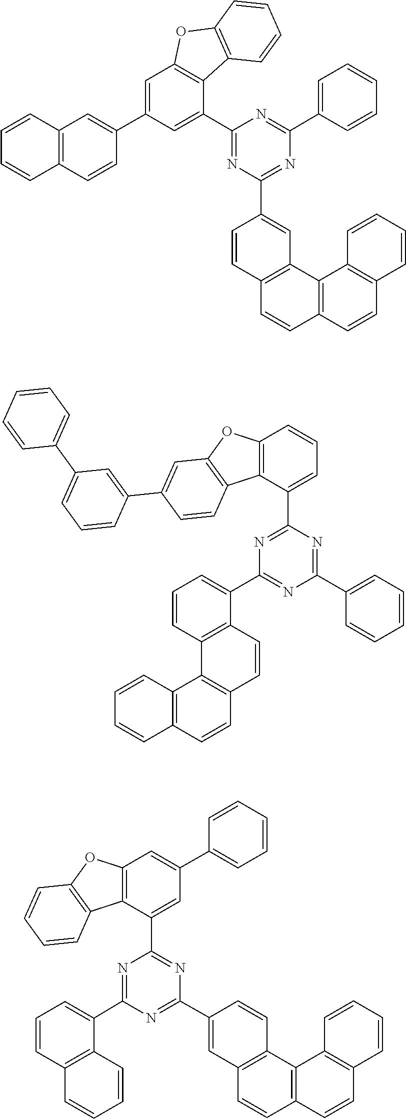 [SMILES: c1ccc(-c2nc(-c3ccc4ccc5ccc6ccccc6c5c4c3)nc(-c3cc(-c4ccc5ccccc5c4)cc4oc5ccccc5c34)n2)cc1 ; c1ccc(-c2cccc(-c3ccc4c(c3)oc3cccc(-c5nc(-c6ccccc6)nc(-c6cccc7c6ccc6ccc8ccccc8c67)n5)c34)c2)cc1 ; c1ccc(-c2cc(-c3nc(-c4ccc5c(ccc6ccc7ccccc7c65)c4)nc(-c4cccc5ccccc45)n3)c3c(c2)oc2ccccc23)cc1]

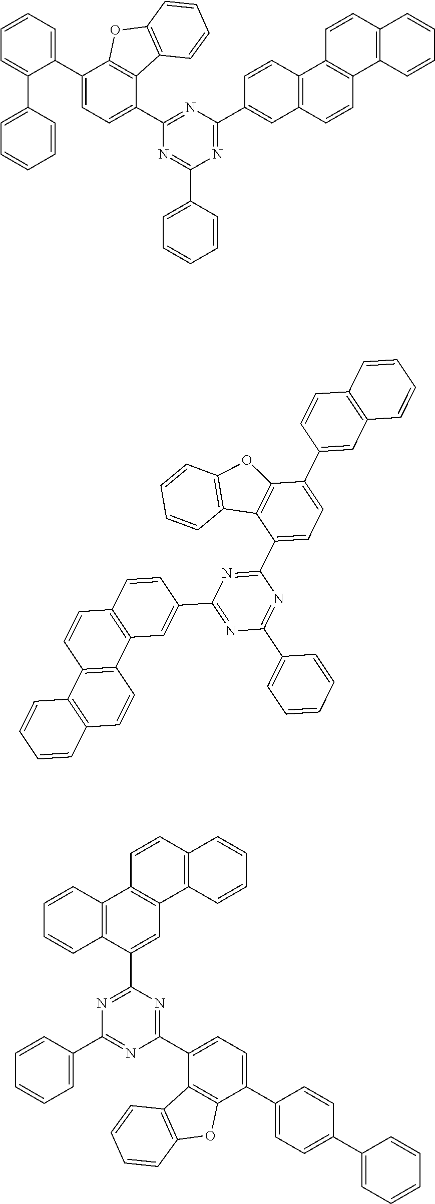 [SMILES: c1ccc(-c2nc(-c3ccc4c(ccc5c6ccccc6ccc45)c3)nc(-c3ccc(-c4ccccc4-c4ccccc4)c4oc5ccccc5c34)n2)cc1 ; c1ccc(-c2nc(-c3ccc4ccc5c6ccccc6ccc5c4c3)nc(-c3ccc(-c4ccc5ccccc5c4)c4oc5ccccc5c34)n2)cc1 ; c1ccc(-c2ccc(-c3ccc(-c4nc(-c5ccccc5)nc(-c5cc6c7ccccc7ccc6c6ccccc56)n4)c4c3oc3ccccc34)cc2)cc1]

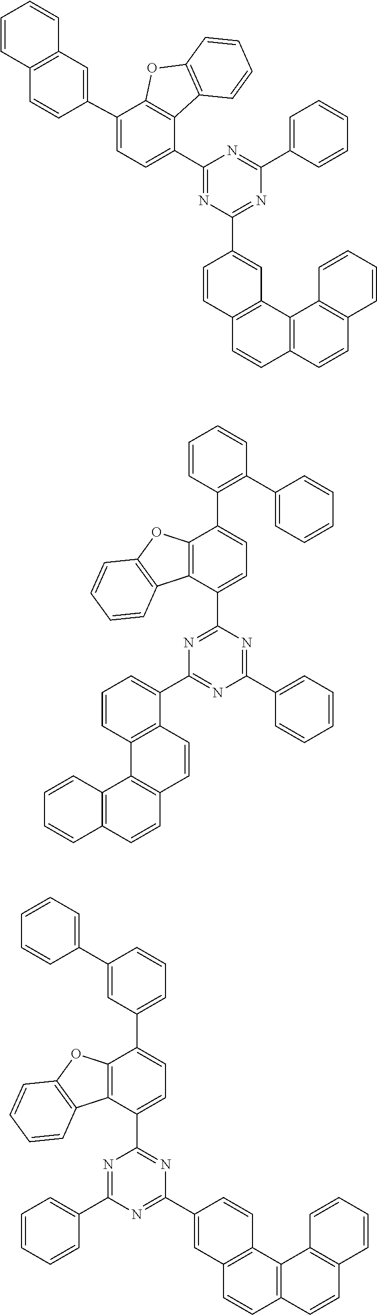 [SMILES: c1ccc(-c2nc(-c3ccc4ccc5ccc6ccccc6c5c4c3)nc(-c3ccc(-c4ccc5ccccc5c4)c4oc5ccccc5c34)n2)cc1 ; c1ccc(-c2nc(-c3cccc4c3ccc3ccc5ccccc5c34)nc(-c3ccc(-c4ccccc4-c4ccccc4)c4oc5ccccc5c34)n2)cc1 ; c1ccc(-c2cccc(-c3ccc(-c4nc(-c5ccccc5)nc(-c5ccc6c(ccc7ccc8ccccc8c76)c5)n4)c4c3oc3ccccc34)c2)cc1]

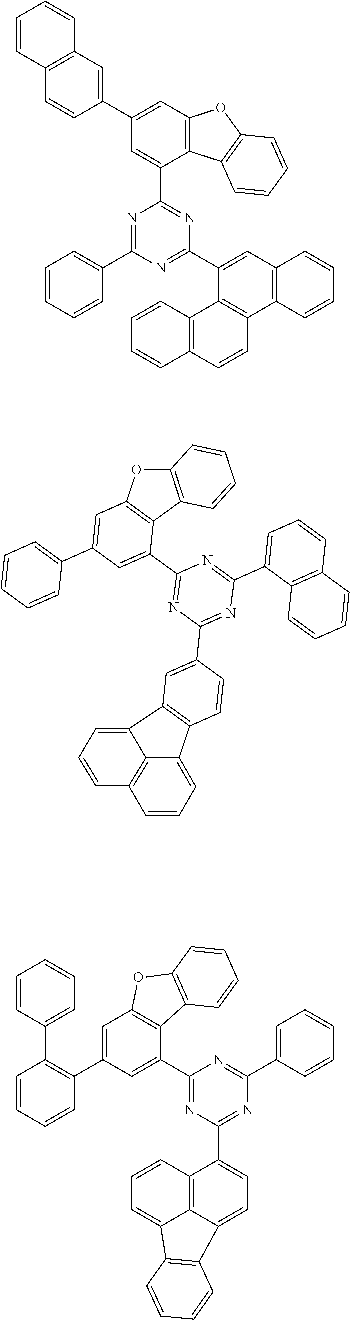 [SMILES: c1ccc(-c2nc(-c3cc(-c4ccc5ccccc5c4)cc4oc5ccccc5c34)nc(-c3cc4ccccc4c4ccc5ccccc5c34)n2)cc1 ; c1ccc(-c2cc(-c3nc(-c4ccc5c(c4)-c4cccc6cccc-5c46)nc(-c4cccc5ccccc45)n3)c3c(c2)oc2ccccc23)cc1 ; c1ccc(-c2nc(-c3ccc4c5c(cccc35)-c3ccccc3-4)nc(-c3cc(-c4ccccc4-c4ccccc4)cc4oc5ccccc5c34)n2)cc1]

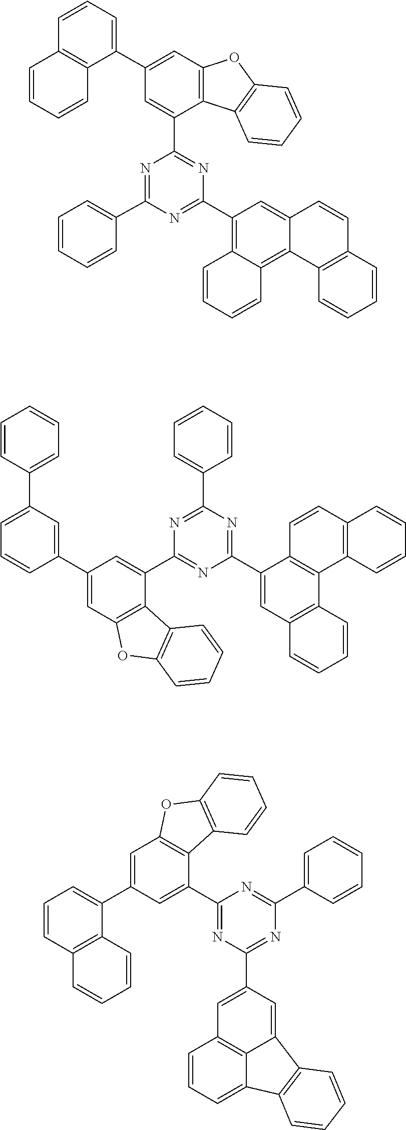 [SMILES: c1ccc(-c2nc(-c3cc4ccc5ccccc5c4c4ccccc34)nc(-c3cc(-c4cccc5ccccc45)cc4oc5ccccc5c34)n2)cc1 ; c1ccc(-c2cccc(-c3cc(-c4nc(-c5ccccc5)nc(-c5cc6ccccc6c6c5ccc5ccccc56)n4)c4c(c3)oc3ccccc34)c2)cc1 ; c1ccc(-c2nc(-c3cc4c5c(cccc5c3)-c3ccccc3-4)nc(-c3cc(-c4cccc5ccccc45)cc4oc5ccccc5c34)n2)cc1]

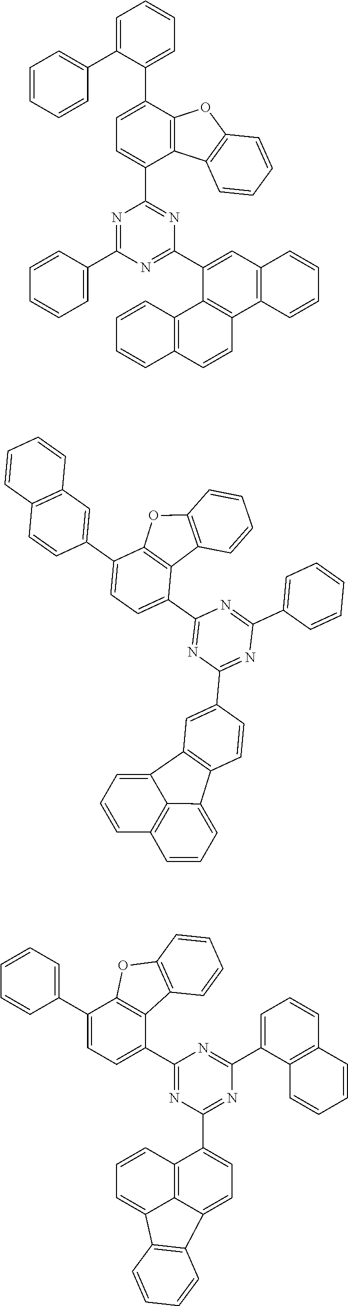 [SMILES: c1ccc(-c2nc(-c3ccc(-c4ccccc4-c4ccccc4)c4oc5ccccc5c34)nc(-c3cc4ccccc4c4ccc5ccccc5c34)n2)cc1 ; c1ccc(-c2nc(-c3ccc4c(c3)-c3cccc5cccc-4c35)nc(-c3ccc(-c4ccc5ccccc5c4)c4oc5ccccc5c34)n2)cc1 ; c1ccc(-c2ccc(-c3nc(-c4cccc5ccccc45)nc(-c4ccc5c6c(cccc46)-c4ccccc4-5)n3)c3c2oc2ccccc23)cc1]

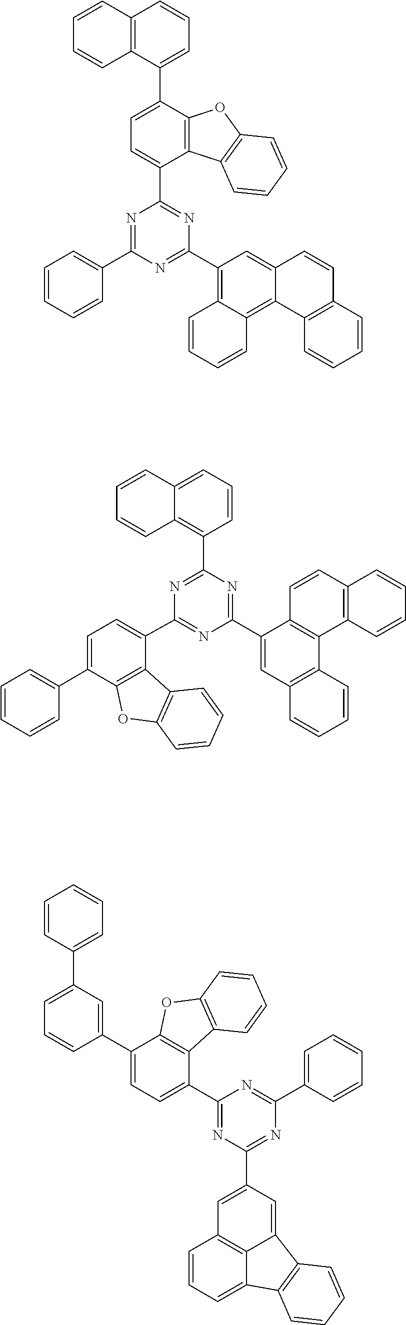 [SMILES: c1ccc(-c2nc(-c3cc4ccc5ccccc5c4c4ccccc34)nc(-c3ccc(-c4cccc5ccccc45)c4oc5ccccc5c34)n2)cc1 ; c1ccc(-c2ccc(-c3nc(-c4cccc5ccccc45)nc(-c4cc5ccccc5c5c4ccc4ccccc45)n3)c3c2oc2ccccc23)cc1 ; c1ccc(-c2cccc(-c3ccc(-c4nc(-c5ccccc5)nc(-c5cc6c7c(cccc7c5)-c5ccccc5-6)n4)c4c3oc3ccccc34)c2)cc1]

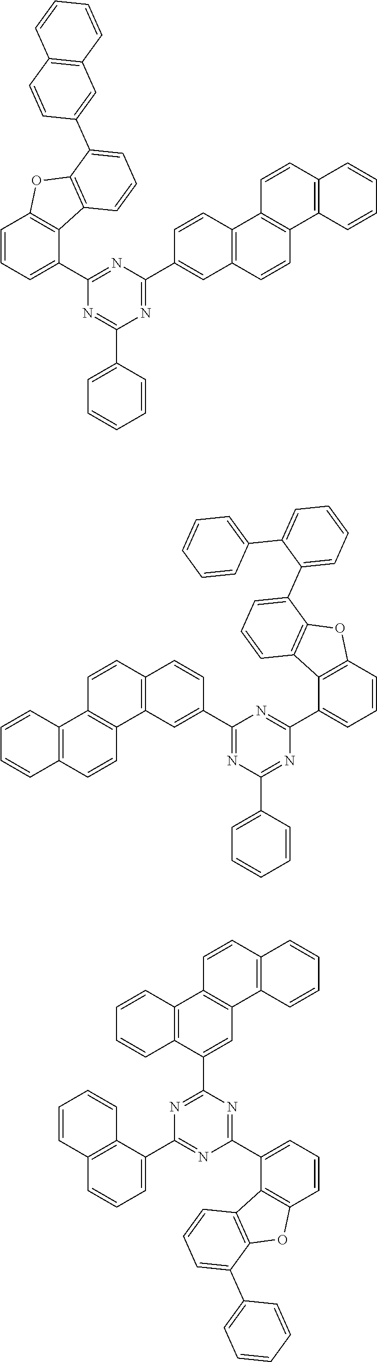 [SMILES: c1ccc(-c2nc(-c3ccc4c(ccc5c6ccccc6ccc45)c3)nc(-c3cccc4oc5c(-c6ccc7ccccc7c6)cccc5c34)n2)cc1 ; c1ccc(-c2nc(-c3ccc4ccc5c6ccccc6ccc5c4c3)nc(-c3cccc4oc5c(-c6ccccc6-c6ccccc6)cccc5c34)n2)cc1 ; c1ccc(-c2cccc3c2oc2cccc(-c4nc(-c5cccc6ccccc56)nc(-c5cc6c7ccccc7ccc6c6ccccc56)n4)c23)cc1]

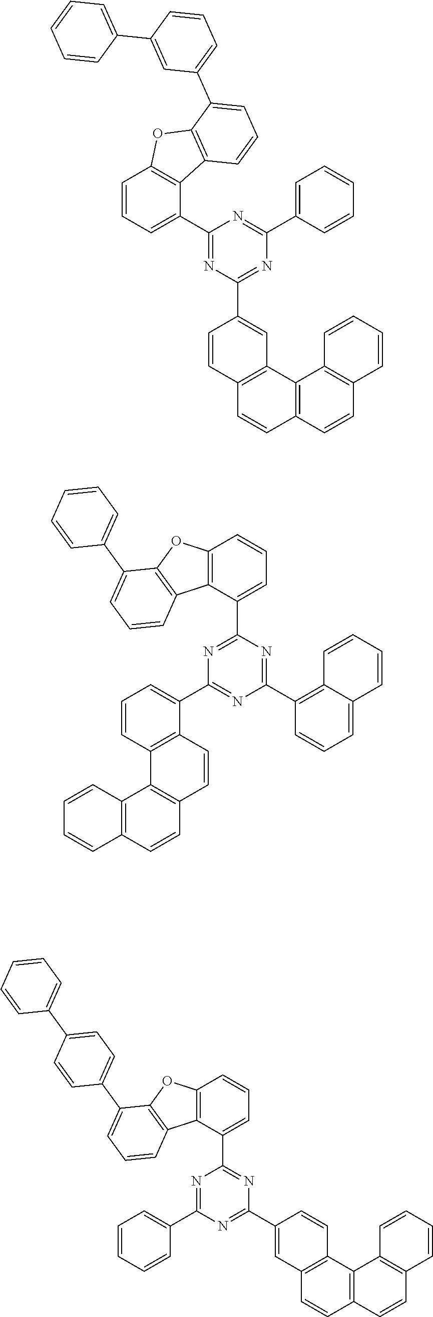 [SMILES: c1ccc(-c2cccc(-c3cccc4c3oc3cccc(-c5nc(-c6ccccc6)nc(-c6ccc7ccc8ccc9ccccc9c8c7c6)n5)c34)c2)cc1 ; c1ccc(-c2cccc3c2oc2cccc(-c4nc(-c5cccc6ccccc56)nc(-c5cccc6c5ccc5ccc7ccccc7c56)n4)c23)cc1 ; c1ccc(-c2ccc(-c3cccc4c3oc3cccc(-c5nc(-c6ccccc6)nc(-c6ccc7c(ccc8ccc9ccccc9c87)c6)n5)c34)cc2)cc1]

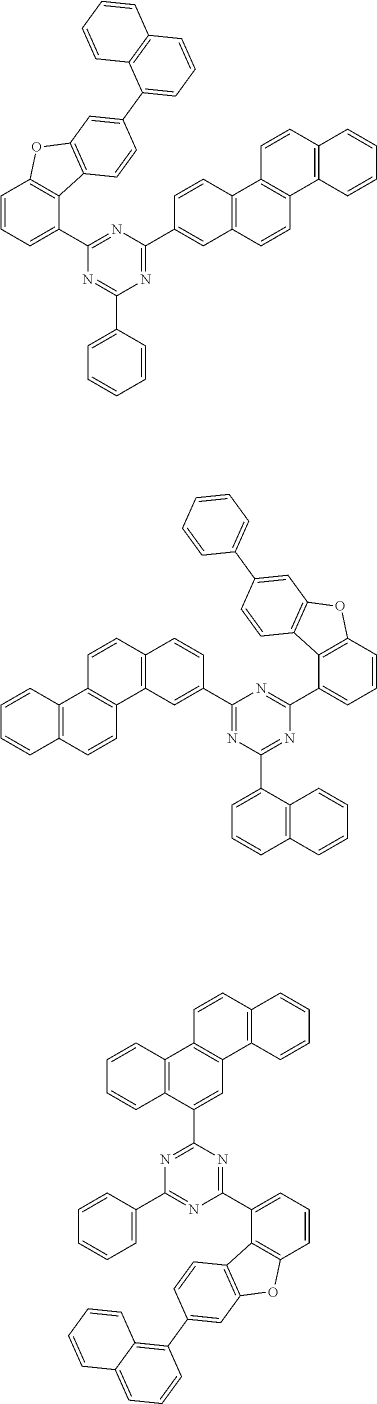 [SMILES: c1ccc(-c2nc(-c3ccc4c(ccc5c6ccccc6ccc45)c3)nc(-c3cccc4oc5cc(-c6cccc7ccccc67)ccc5c34)n2)cc1 ; c1ccc(-c2ccc3c(c2)oc2cccc(-c4nc(-c5ccc6ccc7c8ccccc8ccc7c6c5)nc(-c5cccc6ccccc56)n4)c23)cc1 ; c1ccc(-c2nc(-c3cc4c5ccccc5ccc4c4ccccc34)nc(-c3cccc4oc5cc(-c6cccc7ccccc67)ccc5c34)n2)cc1]

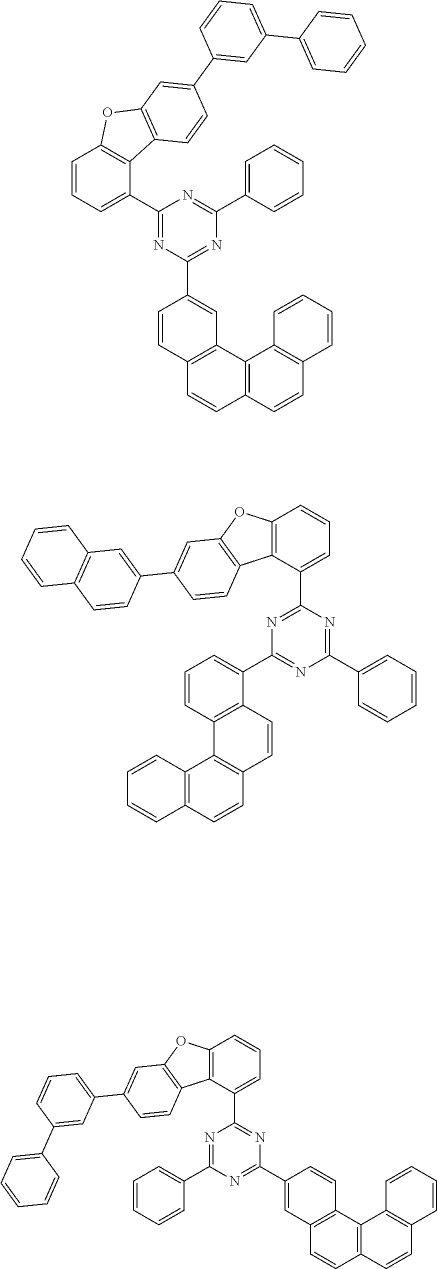 [SMILES: c1ccc(-c2cccc(-c3ccc4c(c3)oc3cccc(-c5nc(-c6ccccc6)nc(-c6ccc7ccc8ccc9ccccc9c8c7c6)n5)c34)c2)cc1 ; c1ccc(-c2nc(-c3cccc4c3ccc3ccc5ccccc5c34)nc(-c3cccc4oc5cc(-c6ccc7ccccc7c6)ccc5c34)n2)cc1 ; c1ccc(-c2cccc(-c3ccc4c(c3)oc3cccc(-c5nc(-c6ccccc6)nc(-c6ccc7c(ccc8ccc9ccccc9c87)c6)n5)c34)c2)cc1]

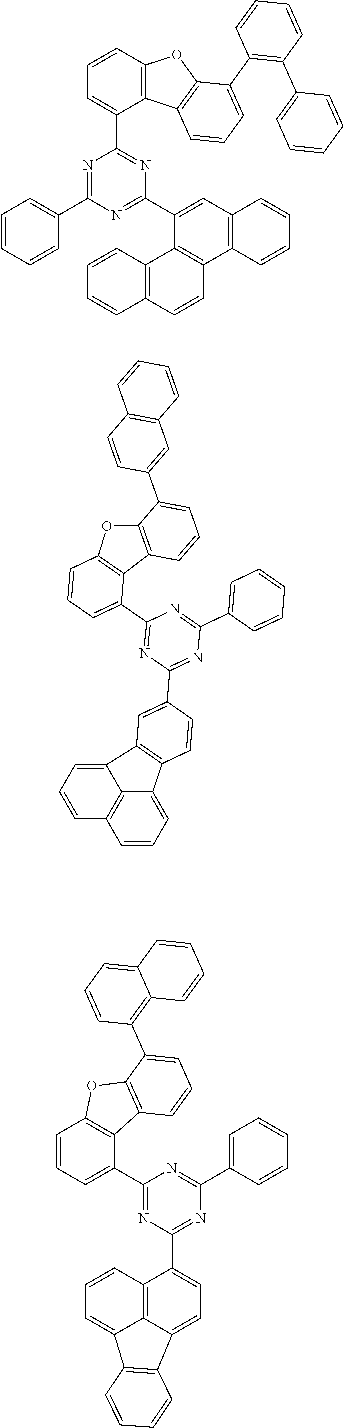 [SMILES: c1ccc(-c2nc(-c3cccc4oc5c(-c6ccccc6-c6ccccc6)cccc5c34)nc(-c3cc4ccccc4c4ccc5ccccc5c34)n2)cc1 ; c1ccc(-c2nc(-c3ccc4c(c3)-c3cccc5cccc-4c35)nc(-c3cccc4oc5c(-c6ccc7ccccc7c6)cccc5c34)n2)cc1 ; c1ccc(-c2nc(-c3ccc4c5c(cccc35)-c3ccccc3-4)nc(-c3cccc4oc5c(-c6cccc7ccccc67)cccc5c34)n2)cc1]

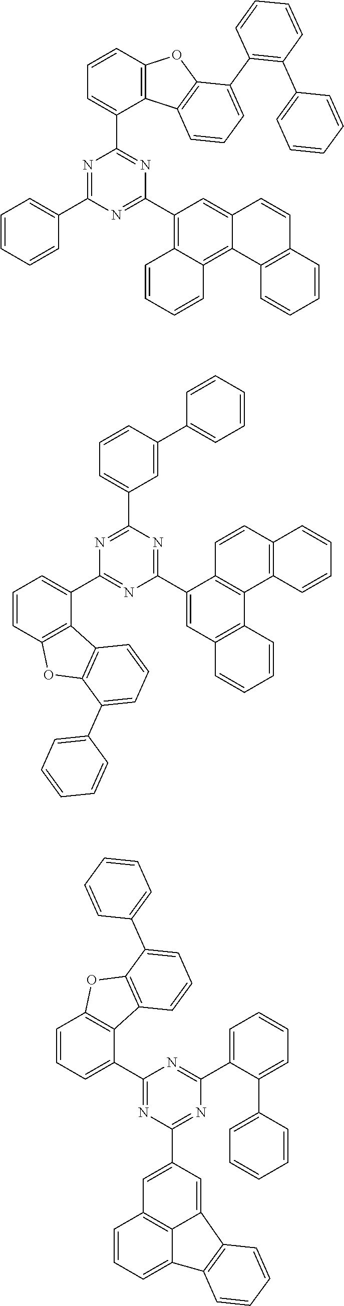 [SMILES: c1ccc(-c2nc(-c3cc4ccc5ccccc5c4c4ccccc34)nc(-c3cccc4oc5c(-c6ccccc6-c6ccccc6)cccc5c34)n2)cc1 ; c1ccc(-c2cccc(-c3nc(-c4cc5ccccc5c5c4ccc4ccccc45)nc(-c4cccc5oc6c(-c7ccccc7)cccc6c45)n3)c2)cc1 ; c1ccc(-c2ccccc2-c2nc(-c3cc4c5c(cccc5c3)-c3ccccc3-4)nc(-c3cccc4oc5c(-c6ccccc6)cccc5c34)n2)cc1]

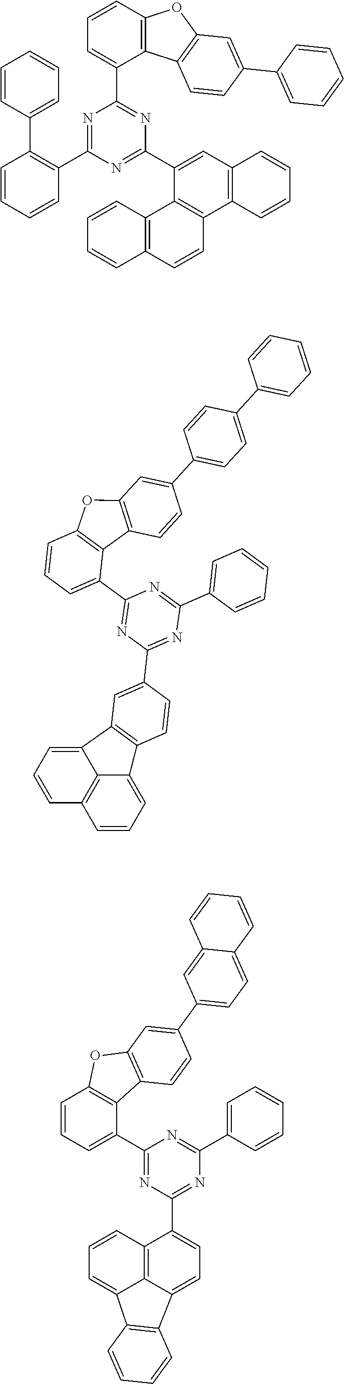 [SMILES: c1ccc(-c2ccc3c(c2)oc2cccc(-c4nc(-c5ccccc5-c5ccccc5)nc(-c5cc6ccccc6c6ccc7ccccc7c56)n4)c23)cc1 ; c1ccc(-c2ccc(-c3ccc4c(c3)oc3cccc(-c5nc(-c6ccccc6)nc(-c6ccc7c(c6)-c6cccc8cccc-7c68)n5)c34)cc2)cc1 ; c1ccc(-c2nc(-c3ccc4c5c(cccc35)-c3ccccc3-4)nc(-c3cccc4oc5cc(-c6ccc7ccccc7c6)ccc5c34)n2)cc1]

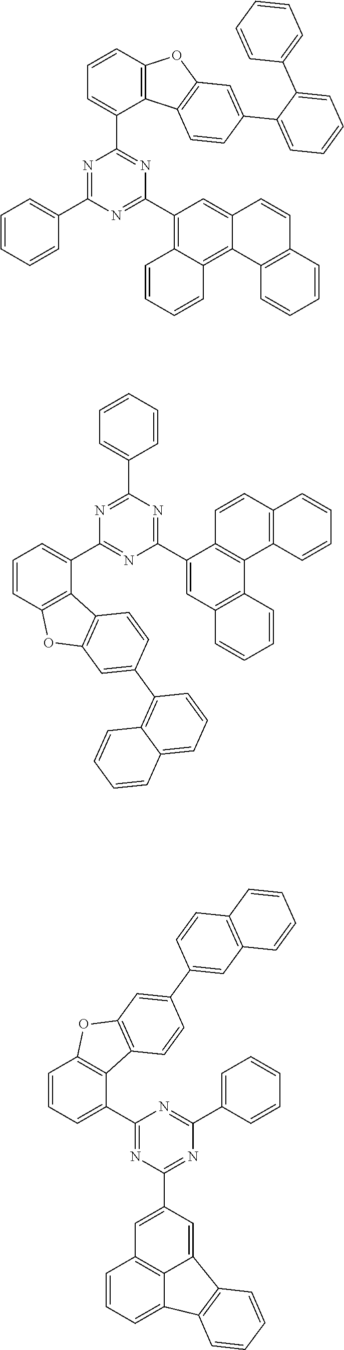 [SMILES: c1ccc(-c2nc(-c3cc4ccc5ccccc5c4c4ccccc34)nc(-c3cccc4oc5cc(-c6ccccc6-c6ccccc6)ccc5c34)n2)cc1 ; c1ccc(-c2nc(-c3cc4ccccc4c4c3ccc3ccccc34)nc(-c3cccc4oc5cc(-c6cccc7ccccc67)ccc5c34)n2)cc1 ; c1ccc(-c2nc(-c3cc4c5c(cccc5c3)-c3ccccc3-4)nc(-c3cccc4oc5cc(-c6ccc7ccccc7c6)ccc5c34)n2)cc1]

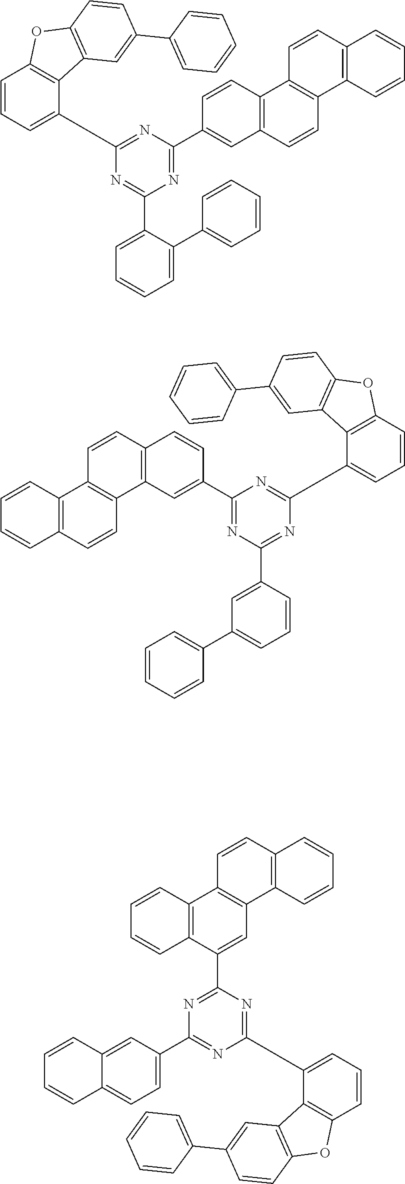 [SMILES: c1ccc(-c2ccc3oc4cccc(-c5nc(-c6ccc7c(ccc8c9ccccc9ccc78)c6)nc(-c6ccccc6-c6ccccc6)n5)c4c3c2)cc1 ; c1ccc(-c2cccc(-c3nc(-c4ccc5ccc6c7ccccc7ccc6c5c4)nc(-c4cccc5oc6ccc(-c7ccccc7)cc6c45)n3)c2)cc1 ; c1ccc(-c2ccc3oc4cccc(-c5nc(-c6ccc7ccccc7c6)nc(-c6cc7c8ccccc8ccc7c7ccccc67)n5)c4c3c2)cc1]

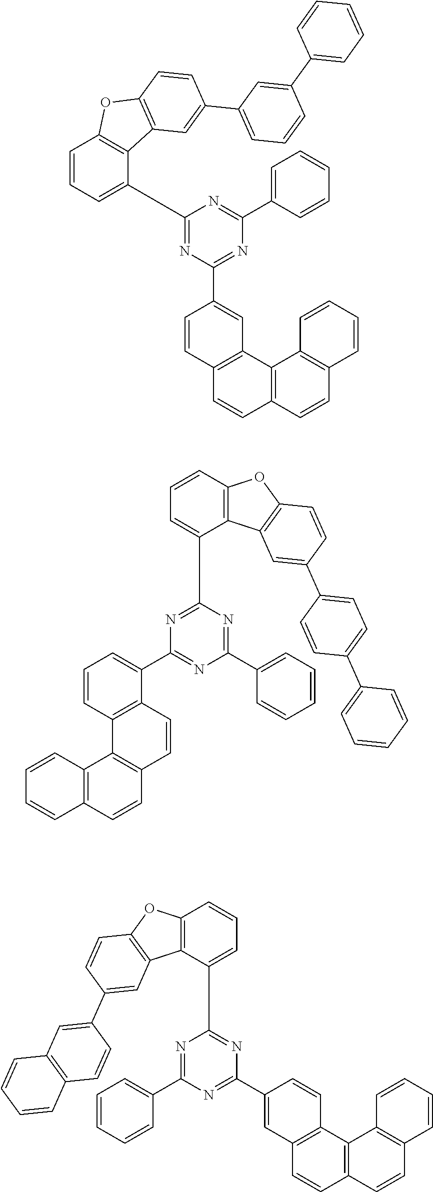 [SMILES: c1ccc(-c2cccc(-c3ccc4oc5cccc(-c6nc(-c7ccccc7)nc(-c7ccc8ccc9ccc%10ccccc%10c9c8c7)n6)c5c4c3)c2)cc1 ; c1ccc(-c2ccc(-c3ccc4oc5cccc(-c6nc(-c7ccccc7)nc(-c7cccc8c7ccc7ccc9ccccc9c78)n6)c5c4c3)cc2)cc1 ; c1ccc(-c2nc(-c3ccc4c(ccc5ccc6ccccc6c54)c3)nc(-c3cccc4oc5ccc(-c6ccc7ccccc7c6)cc5c34)n2)cc1]

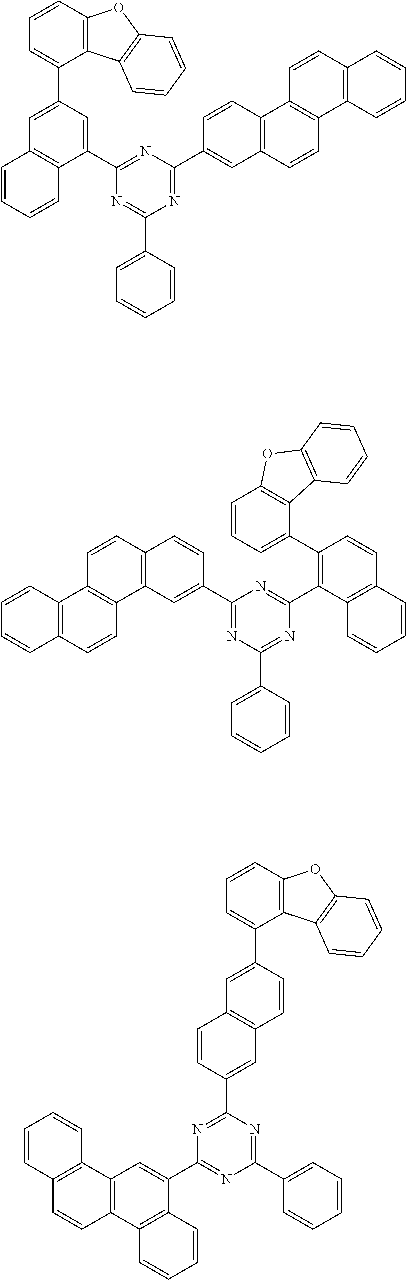 [SMILES: c1ccc(-c2nc(-c3ccc4c(ccc5c6ccccc6ccc45)c3)nc(-c3cc(-c4cccc5oc6ccccc6c45)cc4ccccc34)n2)cc1 ; c1ccc(-c2nc(-c3ccc4ccc5c6ccccc6ccc5c4c3)nc(-c3c(-c4cccc5oc6ccccc6c45)ccc4ccccc34)n2)cc1 ; c1ccc(-c2nc(-c3ccc4cc(-c5cccc6oc7ccccc7c56)ccc4c3)nc(-c3cc4c5ccccc5ccc4c4ccccc34)n2)cc1]

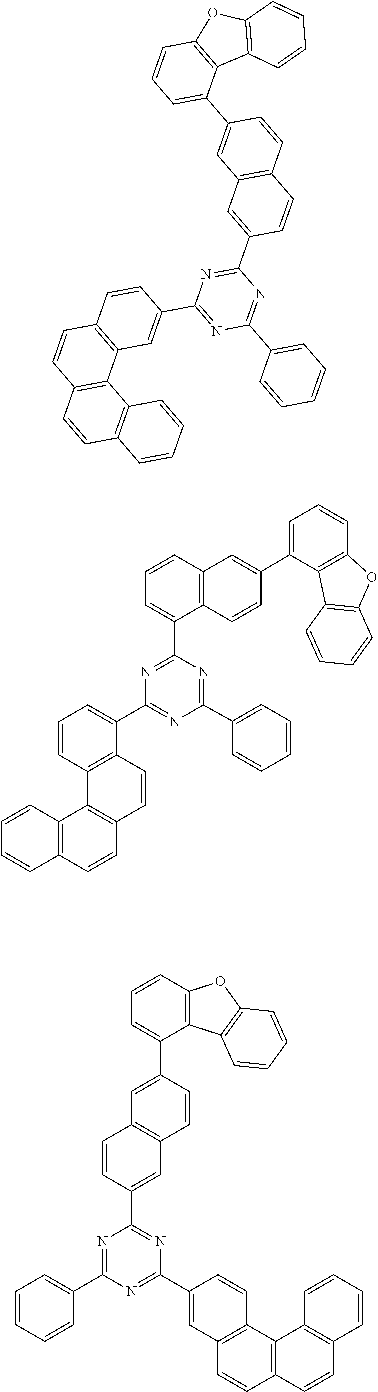 [SMILES: c1ccc(-c2nc(-c3ccc4ccc(-c5cccc6oc7ccccc7c56)cc4c3)nc(-c3ccc4ccc5ccc6ccccc6c5c4c3)n2)cc1 ; c1ccc(-c2nc(-c3cccc4cc(-c5cccc6oc7ccccc7c56)ccc34)nc(-c3cccc4c3ccc3ccc5ccccc5c34)n2)cc1 ; c1ccc(-c2nc(-c3ccc4cc(-c5cccc6oc7ccccc7c56)ccc4c3)nc(-c3ccc4c(ccc5ccc6ccccc6c54)c3)n2)cc1]

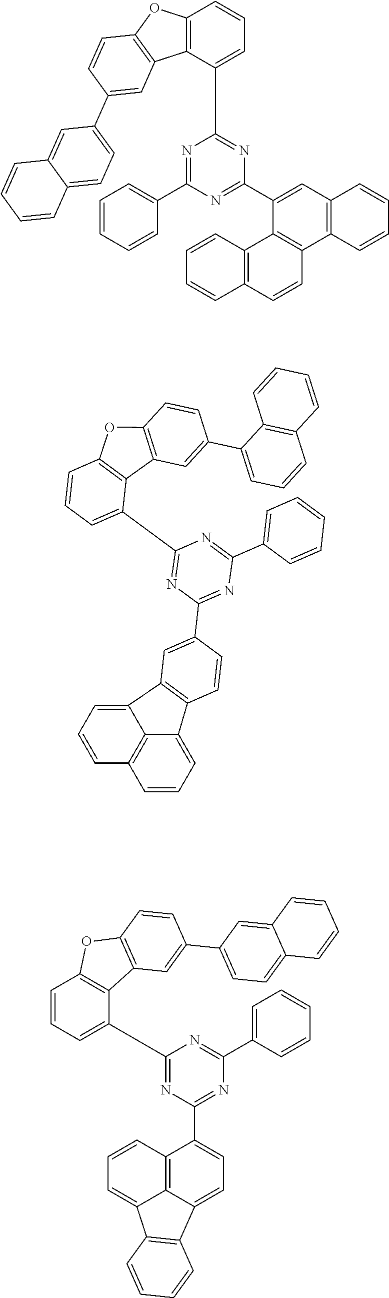 [SMILES: c1ccc(-c2nc(-c3cccc4oc5ccc(-c6ccc7ccccc7c6)cc5c34)nc(-c3cc4ccccc4c4ccc5ccccc5c34)n2)cc1 ; c1ccc(-c2nc(-c3ccc4c(c3)-c3cccc5cccc-4c35)nc(-c3cccc4oc5ccc(-c6cccc7ccccc67)cc5c34)n2)cc1 ; c1ccc(-c2nc(-c3ccc4c5c(cccc35)-c3ccccc3-4)nc(-c3cccc4oc5ccc(-c6ccc7ccccc7c6)cc5c34)n2)cc1]

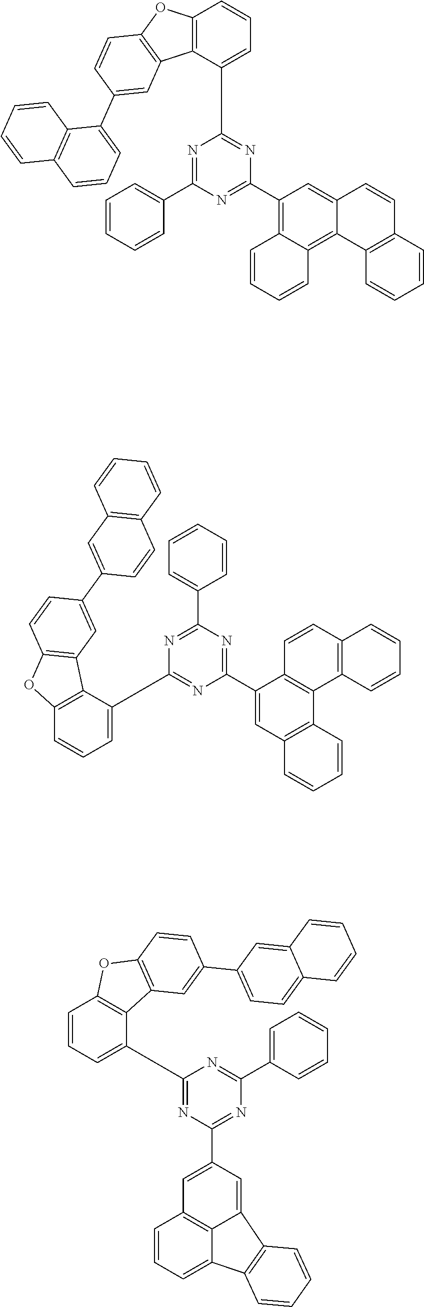 [SMILES: c1ccc(-c2nc(-c3cc4ccc5ccccc5c4c4ccccc34)nc(-c3cccc4oc5ccc(-c6cccc7ccccc67)cc5c34)n2)cc1 ; c1ccc(-c2nc(-c3cc4ccccc4c4c3ccc3ccccc34)nc(-c3cccc4oc5ccc(-c6ccc7ccccc7c6)cc5c34)n2)cc1 ; c1ccc(-c2nc(-c3cc4c5c(cccc5c3)-c3ccccc3-4)nc(-c3cccc4oc5ccc(-c6ccc7ccccc7c6)cc5c34)n2)cc1]

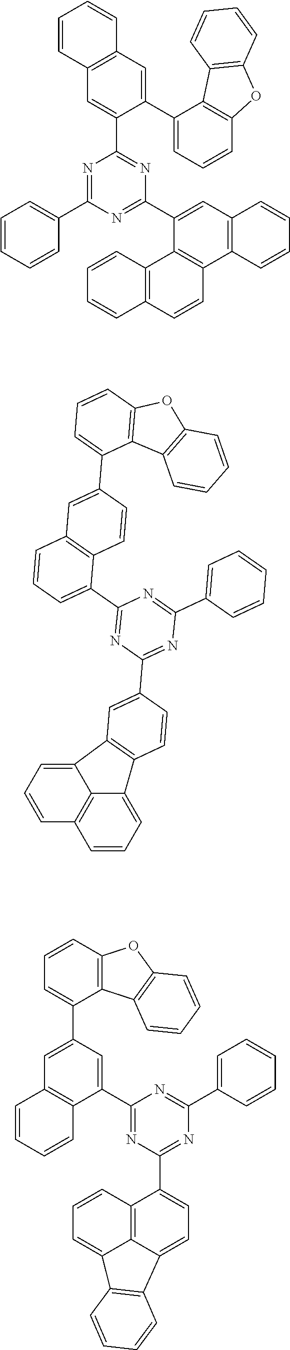 [SMILES: c1ccc(-c2nc(-c3cc4ccccc4cc3-c3cccc4oc5ccccc5c34)nc(-c3cc4ccccc4c4ccc5ccccc5c34)n2)cc1 ; c1ccc(-c2nc(-c3ccc4c(c3)-c3cccc5cccc-4c35)nc(-c3cccc4cc(-c5cccc6oc7ccccc7c56)ccc34)n2)cc1 ; c1ccc(-c2nc(-c3cc(-c4cccc5oc6ccccc6c45)cc4ccccc34)nc(-c3ccc4c5c(cccc35)-c3ccccc3-4)n2)cc1]

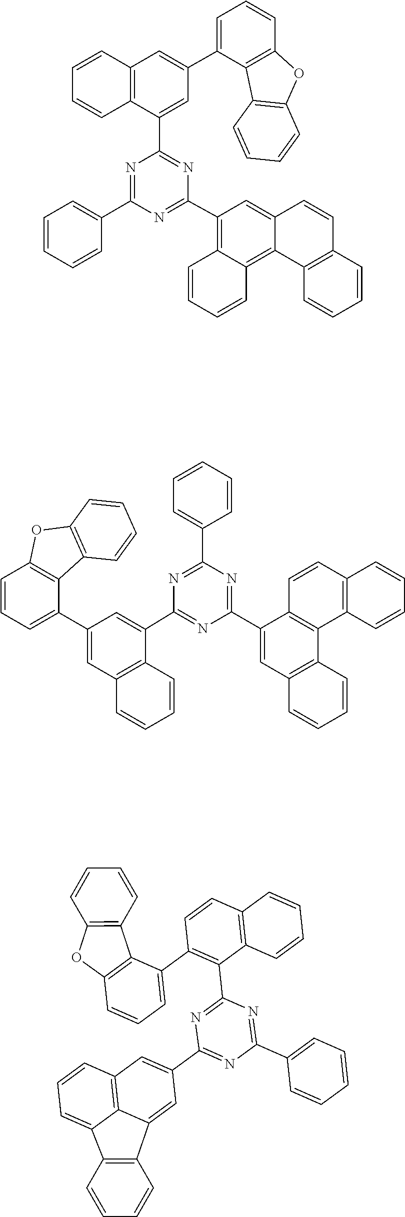 [SMILES: c1ccc(-c2nc(-c3cc(-c4cccc5oc6ccccc6c45)cc4ccccc34)nc(-c3cc4ccc5ccccc5c4c4ccccc34)n2)cc1 ; c1ccc(-c2nc(-c3cc(-c4cccc5oc6ccccc6c45)cc4ccccc34)nc(-c3cc4ccccc4c4c3ccc3ccccc34)n2)cc1 ; c1ccc(-c2nc(-c3cc4c5c(cccc5c3)-c3ccccc3-4)nc(-c3c(-c4cccc5oc6ccccc6c45)ccc4ccccc34)n2)cc1]

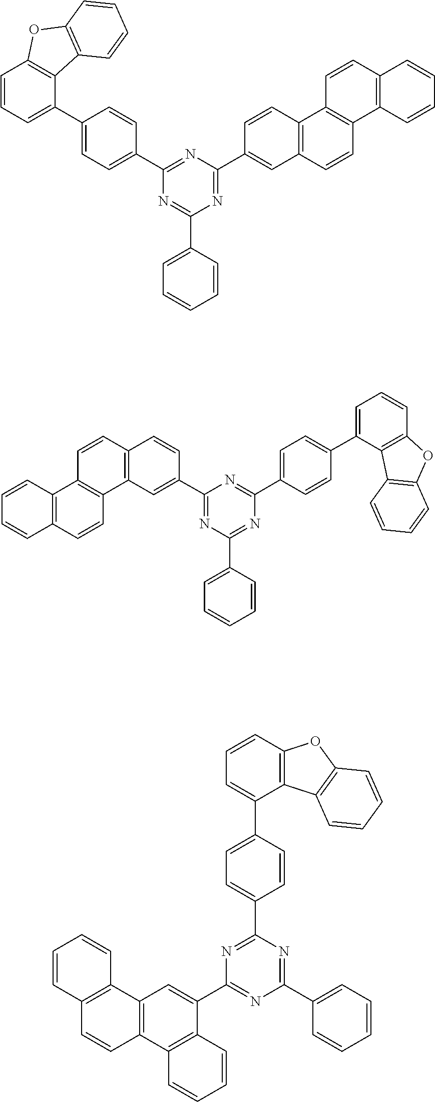 [SMILES: c1ccc(-c2nc(-c3ccc(-c4cccc5oc6ccccc6c45)cc3)nc(-c3ccc4c(ccc5c6ccccc6ccc45)c3)n2)cc1 ; c1ccc(-c2nc(-c3ccc(-c4cccc5oc6ccccc6c45)cc3)nc(-c3ccc4ccc5c6ccccc6ccc5c4c3)n2)cc1 ; c1ccc(-c2nc(-c3ccc(-c4cccc5oc6ccccc6c45)cc3)nc(-c3cc4c5ccccc5ccc4c4ccccc34)n2)cc1]

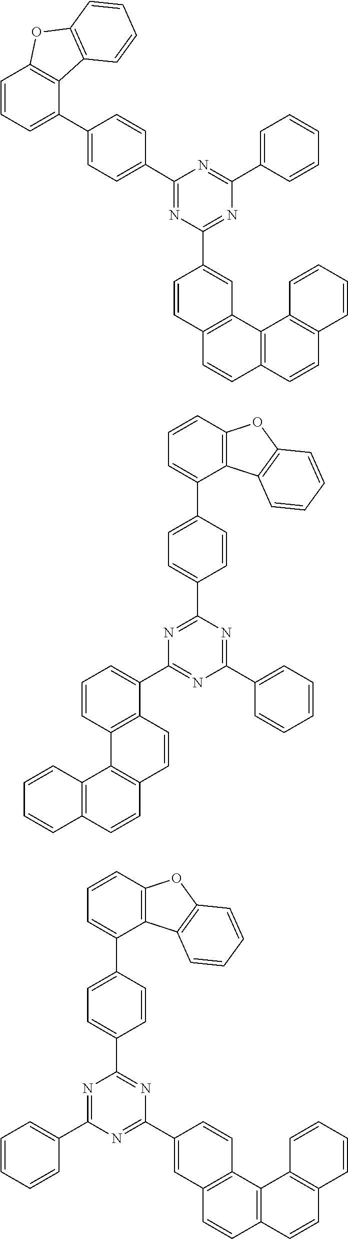

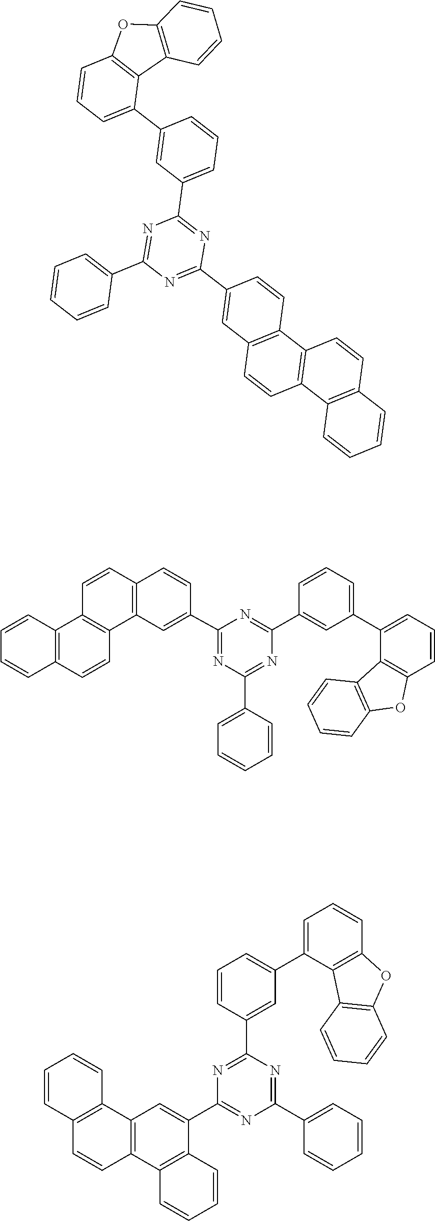 [SMILES: c1ccc(-c2nc(-c3cccc(-c4cccc5oc6ccccc6c45)c3)nc(-c3ccc4c(ccc5c6ccccc6ccc45)c3)n2)cc1 ; c1ccc(-c2nc(-c3cccc(-c4cccc5oc6ccccc6c45)c3)nc(-c3ccc4ccc5c6ccccc6ccc5c4c3)n2)cc1 ; c1ccc(-c2nc(-c3cccc(-c4cccc5oc6ccccc6c45)c3)nc(-c3cc4c5ccccc5ccc4c4ccccc34)n2)cc1]

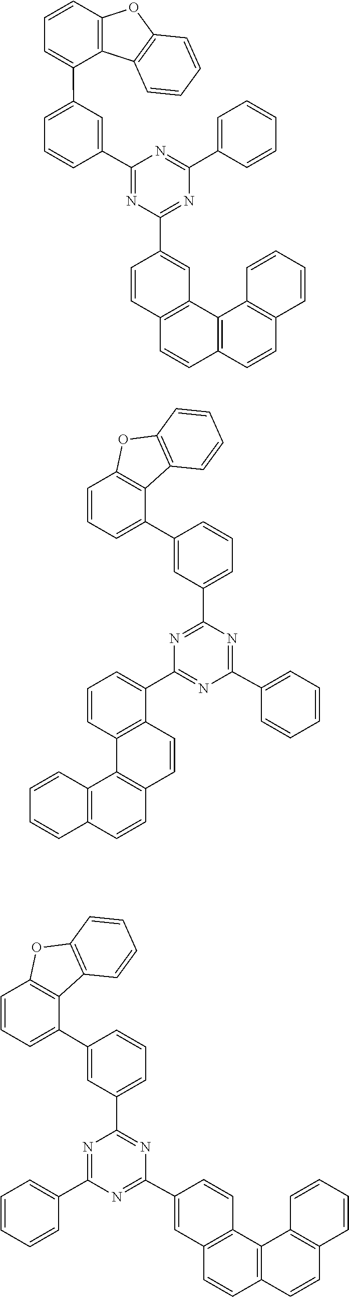

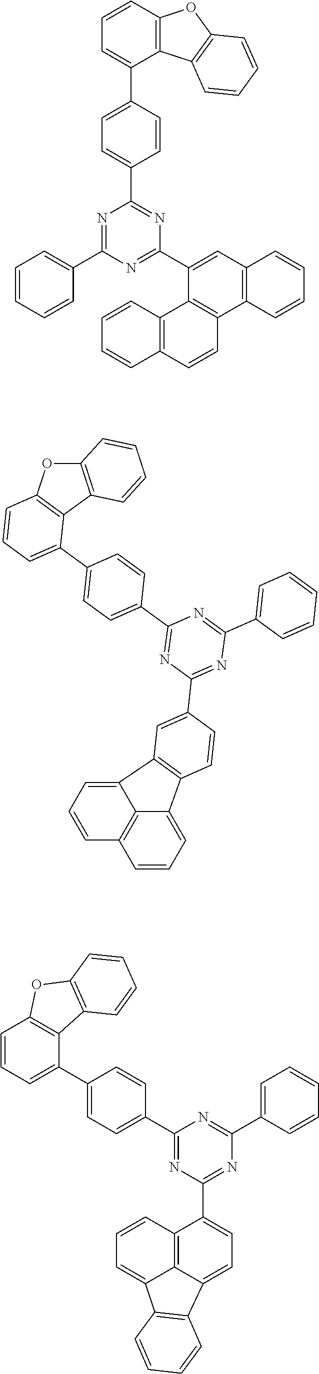 [SMILES: c1ccc(-c2nc(-c3ccc(-c4cccc5oc6ccccc6c45)cc3)nc(-c3cc4ccccc4c4ccc5ccccc5c34)n2)cc1 ; c1ccc(-c2nc(-c3ccc(-c4cccc5oc6ccccc6c45)cc3)nc(-c3ccc4c(c3)-c3cccc5cccc-4c35)n2)cc1 ; c1ccc(-c2nc(-c3ccc(-c4cccc5oc6ccccc6c45)cc3)nc(-c3ccc4c5c(cccc35)-c3ccccc3-4)n2)cc1]

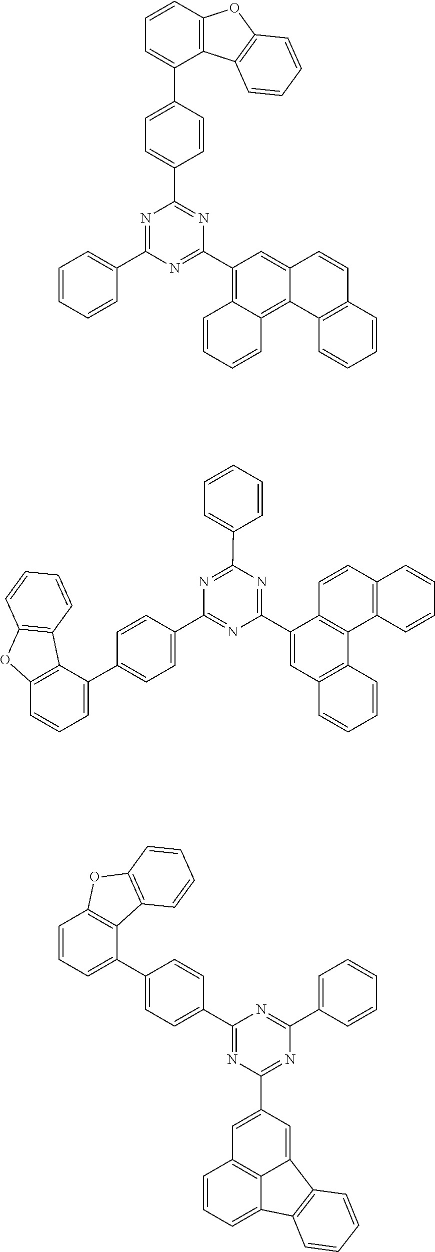 [SMILES: c1ccc(-c2nc(-c3ccc(-c4cccc5oc6ccccc6c45)cc3)nc(-c3cc4ccc5ccccc5c4c4ccccc34)n2)cc1 ; c1ccc(-c2nc(-c3ccc(-c4cccc5oc6ccccc6c45)cc3)nc(-c3cc4ccccc4c4c3ccc3ccccc34)n2)cc1 ; c1ccc(-c2nc(-c3ccc(-c4cccc5oc6ccccc6c45)cc3)nc(-c3cc4c5c(cccc5c3)-c3ccccc3-4)n2)cc1]

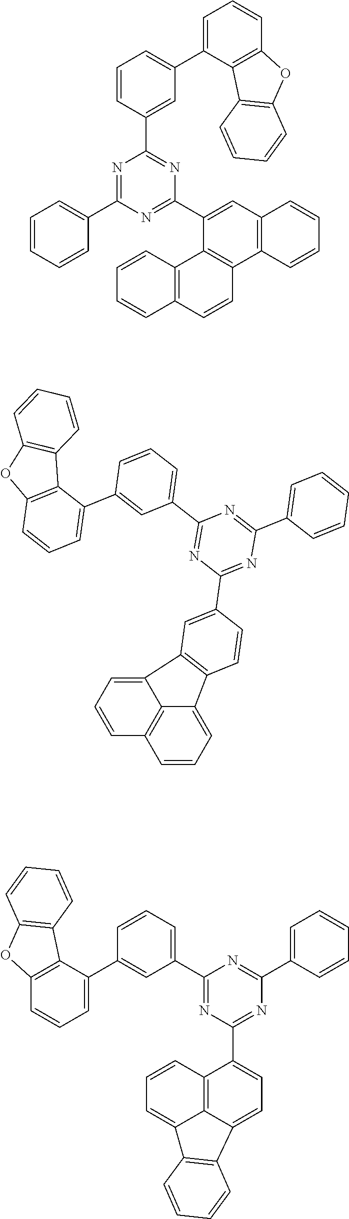 [SMILES: c1ccc(-c2nc(-c3cccc(-c4cccc5oc6ccccc6c45)c3)nc(-c3cc4ccccc4c4ccc5ccccc5c34)n2)cc1 ; c1ccc(-c2nc(-c3cccc(-c4cccc5oc6ccccc6c45)c3)nc(-c3ccc4c(c3)-c3cccc5cccc-4c35)n2)cc1 ; c1ccc(-c2nc(-c3cccc(-c4cccc5oc6ccccc6c45)c3)nc(-c3ccc4c5c(cccc35)-c3ccccc3-4)n2)cc1]

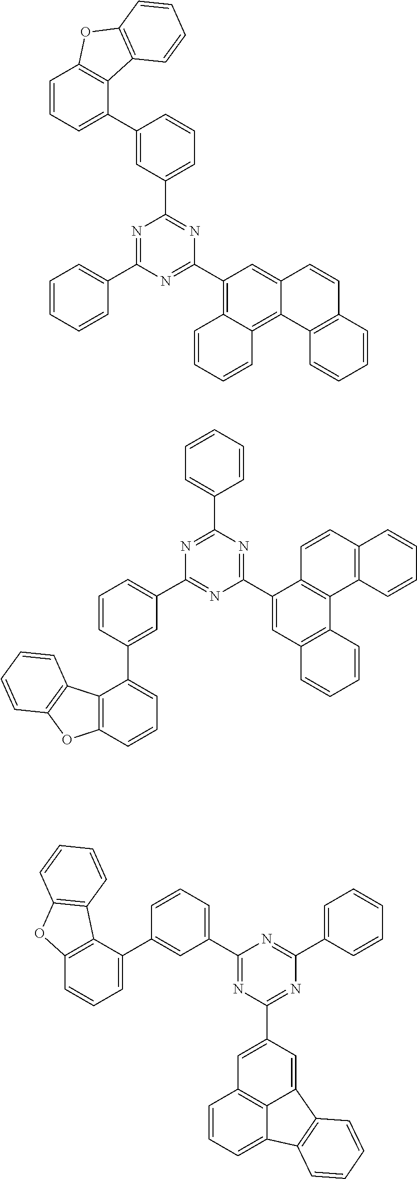 [SMILES: c1ccc(-c2nc(-c3cccc(-c4cccc5oc6ccccc6c45)c3)nc(-c3cc4ccc5ccccc5c4c4ccccc34)n2)cc1 ; c1ccc(-c2nc(-c3cccc(-c4cccc5oc6ccccc6c45)c3)nc(-c3cc4ccccc4c4c3ccc3ccccc34)n2)cc1 ; c1ccc(-c2nc(-c3cccc(-c4cccc5oc6ccccc6c45)c3)nc(-c3cc4c5c(cccc5c3)-c3ccccc3-4)n2)cc1]

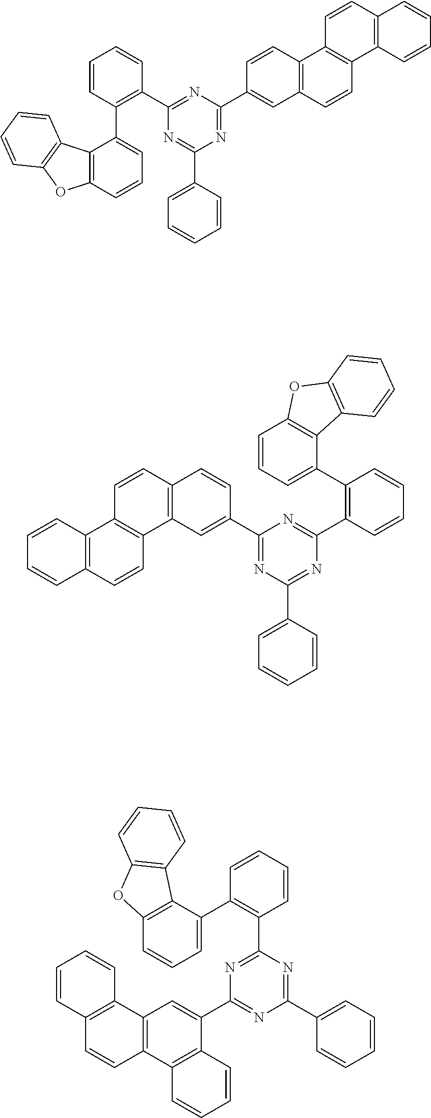 [SMILES: c1ccc(-c2nc(-c3ccc4c(ccc5c6ccccc6ccc45)c3)nc(-c3ccccc3-c3cccc4oc5ccccc5c34)n2)cc1 ; c1ccc(-c2nc(-c3ccc4ccc5c6ccccc6ccc5c4c3)nc(-c3ccccc3-c3cccc4oc5ccccc5c34)n2)cc1 ; c1ccc(-c2nc(-c3ccccc3-c3cccc4oc5ccccc5c34)nc(-c3cc4c5ccccc5ccc4c4ccccc34)n2)cc1]

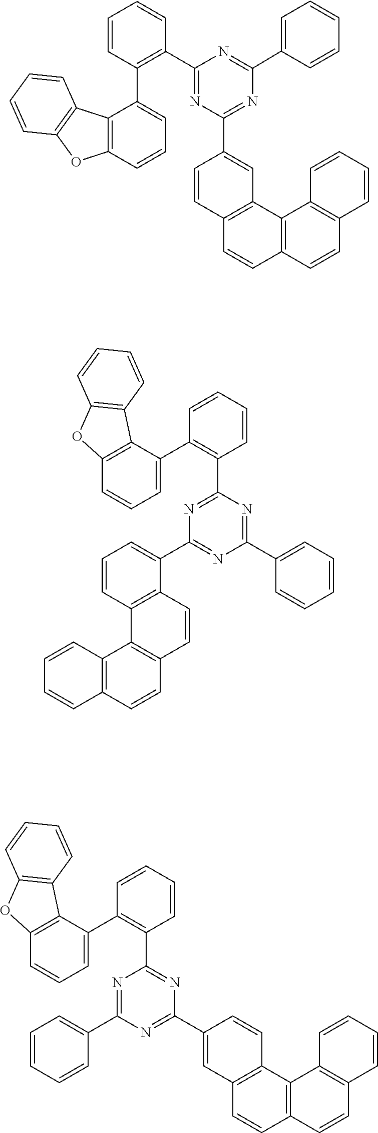 [SMILES: c1ccc(-c2nc(-c3ccc4ccc5ccc6ccccc6c5c4c3)nc(-c3ccccc3-c3cccc4oc5ccccc5c34)n2)cc1 ; c1ccc(-c2nc(-c3ccccc3-c3cccc4oc5ccccc5c34)nc(-c3cccc4c3ccc3ccc5ccccc5c34)n2)cc1 ; c1ccc(-c2nc(-c3ccc4c(ccc5ccc6ccccc6c54)c3)nc(-c3ccccc3-c3cccc4oc5ccccc5c34)n2)cc1]

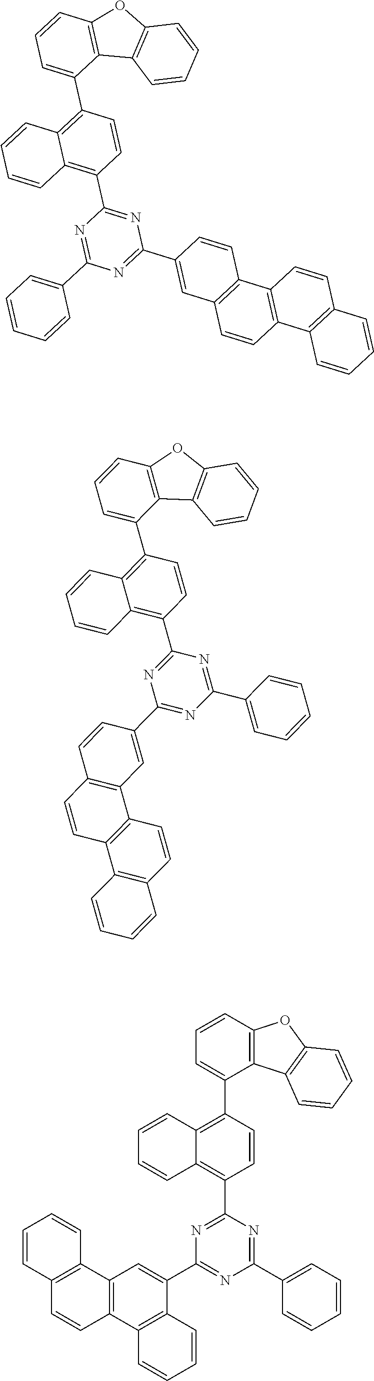 [SMILES: c1ccc(-c2nc(-c3ccc4c(ccc5c6ccccc6ccc45)c3)nc(-c3ccc(-c4cccc5oc6ccccc6c45)c4ccccc34)n2)cc1 ; c1ccc(-c2nc(-c3ccc4ccc5c6ccccc6ccc5c4c3)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cc4c5ccccc5ccc4c4ccccc34)n2)cc1]

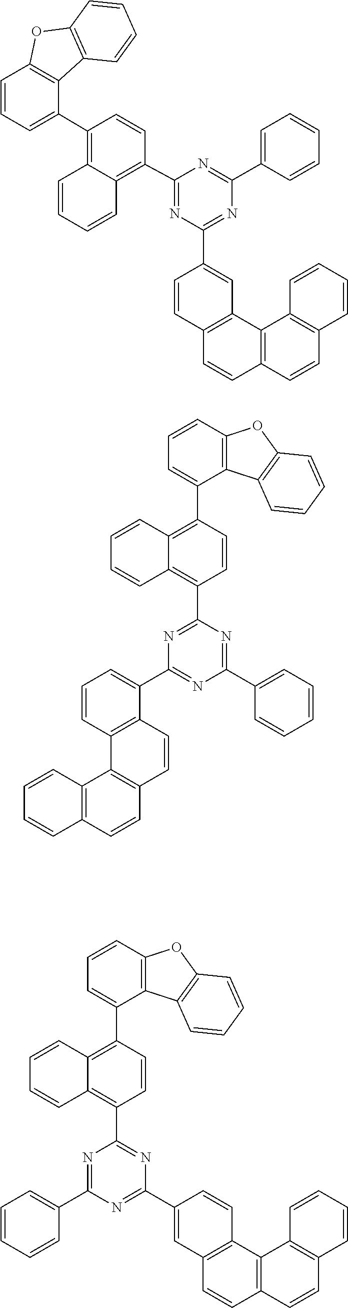 [SMILES: c1ccc(-c2nc(-c3ccc4ccc5ccc6ccccc6c5c4c3)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cccc4c3ccc3ccc5ccccc5c34)n2)cc1 ; c1ccc(-c2nc(-c3ccc4c(ccc5ccc6ccccc6c54)c3)nc(-c3ccc(-c4cccc5oc6ccccc6c45)c4ccccc34)n2)cc1]

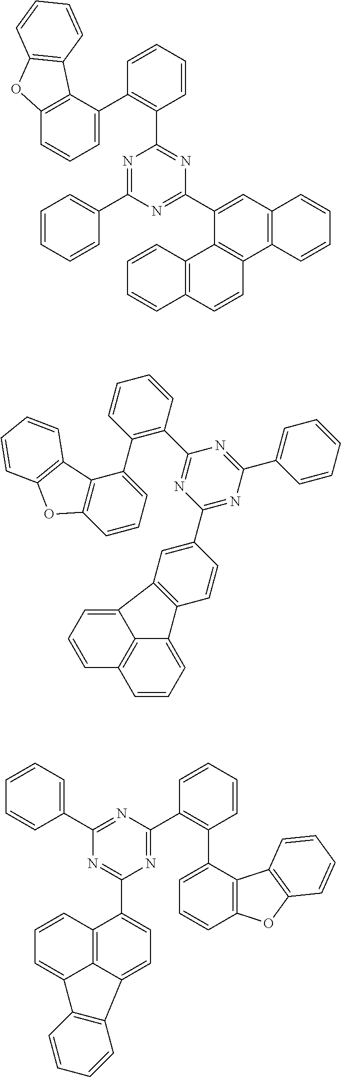 [SMILES: c1ccc(-c2nc(-c3ccccc3-c3cccc4oc5ccccc5c34)nc(-c3cc4ccccc4c4ccc5ccccc5c34)n2)cc1 ; c1ccc(-c2nc(-c3ccc4c(c3)-c3cccc5cccc-4c35)nc(-c3ccccc3-c3cccc4oc5ccccc5c34)n2)cc1 ; c1ccc(-c2nc(-c3ccccc3-c3cccc4oc5ccccc5c34)nc(-c3ccc4c5c(cccc35)-c3ccccc3-4)n2)cc1]

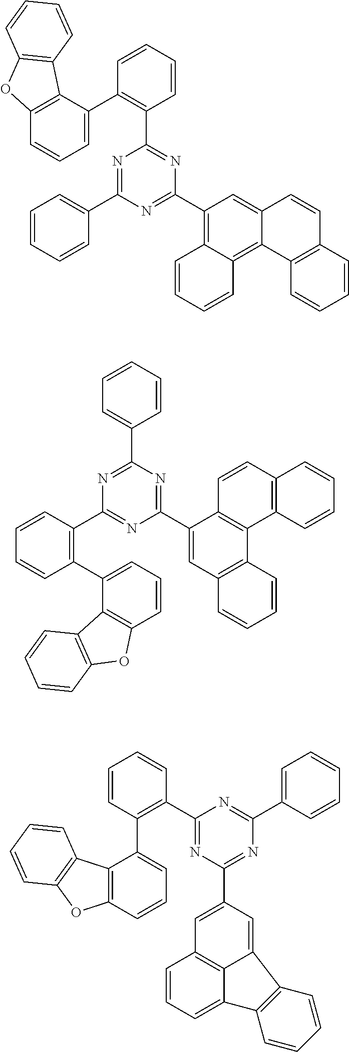 [SMILES: c1ccc(-c2nc(-c3ccccc3-c3cccc4oc5ccccc5c34)nc(-c3cc4ccc5ccccc5c4c4ccccc34)n2)cc1 ; c1ccc(-c2nc(-c3ccccc3-c3cccc4oc5ccccc5c34)nc(-c3cc4ccccc4c4c3ccc3ccccc34)n2)cc1 ; c1ccc(-c2nc(-c3cc4c5c(cccc5c3)-c3ccccc3-4)nc(-c3ccccc3-c3cccc4oc5ccccc5c34)n2)cc1]

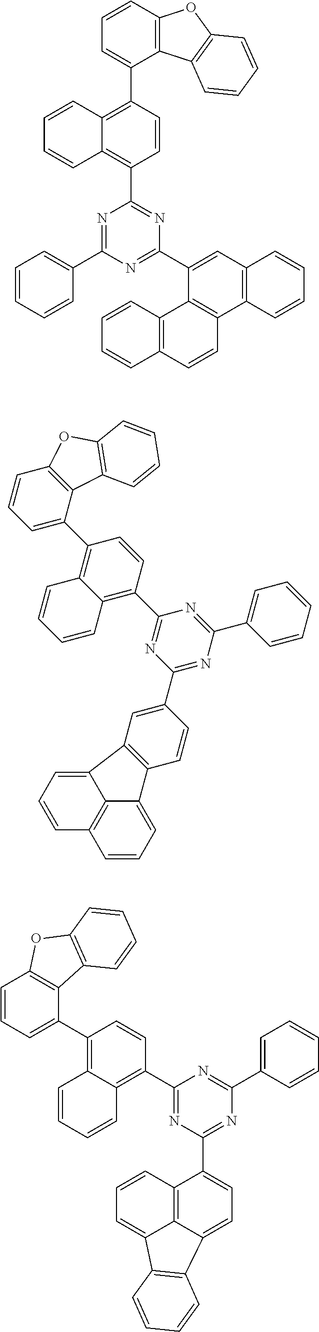 [SMILES: c1ccc(-c2nc(-c3ccc(-c4cccc5oc6ccccc6c45)c4ccccc34)nc(-c3cc4ccccc4c4ccc5ccccc5c34)n2)cc1 ; c1ccc(-c2nc(-c3ccc4c(c3)-c3cccc5cccc-4c35)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3ccc4c5c(cccc35)-c3ccccc3-4)n2)cc1]

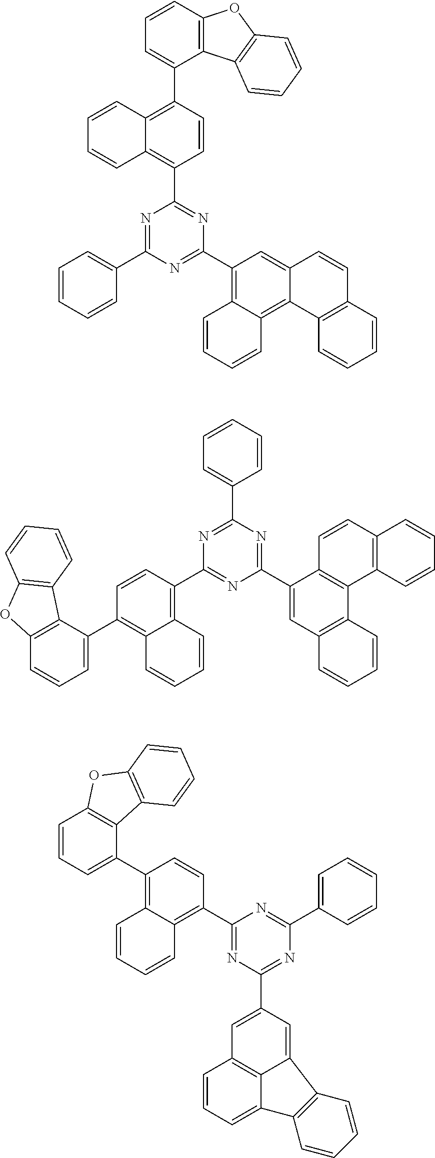 [SMILES: c1ccc(-c2nc(-c3ccc(-c4cccc5oc6ccccc6c45)c4ccccc34)nc(-c3cc4ccc5ccccc5c4c4ccccc34)n2)cc1 ; c1ccc(-c2nc(-c3ccc(-c4cccc5oc6ccccc6c45)c4ccccc34)nc(-c3cc4ccccc4c4c3ccc3ccccc34)n2)cc1 ; c1ccc(-c2nc(-c3cc4c5c(cccc5c3)-c3ccccc3-4)nc(-c3ccc(-c4cccc5oc6ccccc6c45)c4ccccc34)n2)cc1]

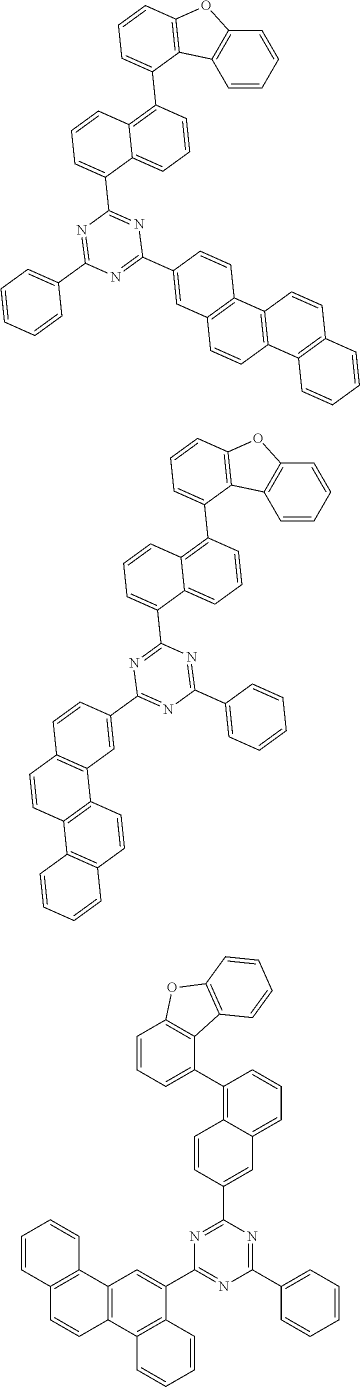 [SMILES: c1ccc(-c2nc(-c3ccc4c(ccc5c6ccccc6ccc45)c3)nc(-c3cccc4c(-c5cccc6oc7ccccc7c56)cccc34)n2)cc1 ; c1ccc(-c2nc(-c3ccc4ccc5c6ccccc6ccc5c4c3)nc(-c3cccc4c(-c5cccc6oc7ccccc7c56)cccc34)n2)cc1 ; c1ccc(-c2nc(-c3ccc4c(-c5cccc6oc7ccccc7c56)cccc4c3)nc(-c3cc4c5ccccc5ccc4c4ccccc34)n2)cc1]

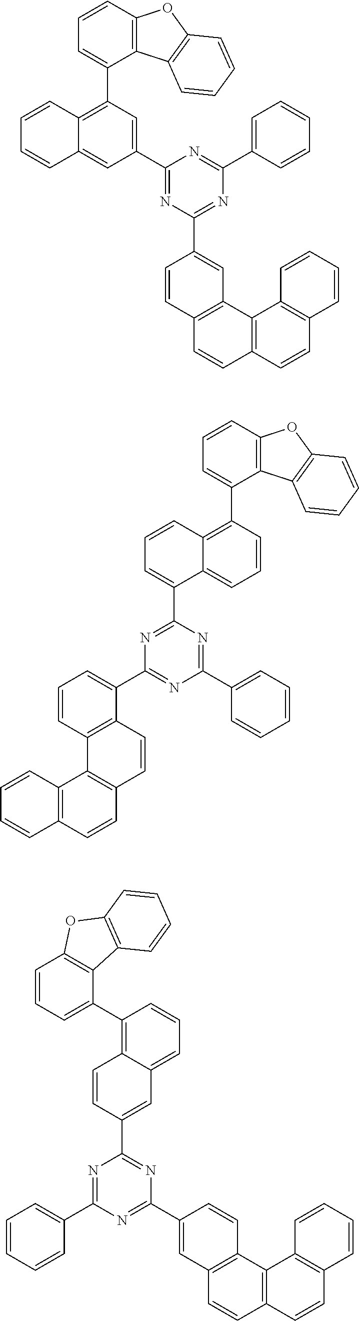 [SMILES: c1ccc(-c2nc(-c3cc(-c4cccc5oc6ccccc6c45)c4ccccc4c3)nc(-c3ccc4ccc5ccc6ccccc6c5c4c3)n2)cc1 ; c1ccc(-c2nc(-c3cccc4c(-c5cccc6oc7ccccc7c56)cccc34)nc(-c3cccc4c3ccc3ccc5ccccc5c34)n2)cc1 ; c1ccc(-c2nc(-c3ccc4c(-c5cccc6oc7ccccc7c56)cccc4c3)nc(-c3ccc4c(ccc5ccc6ccccc6c54)c3)n2)cc1]

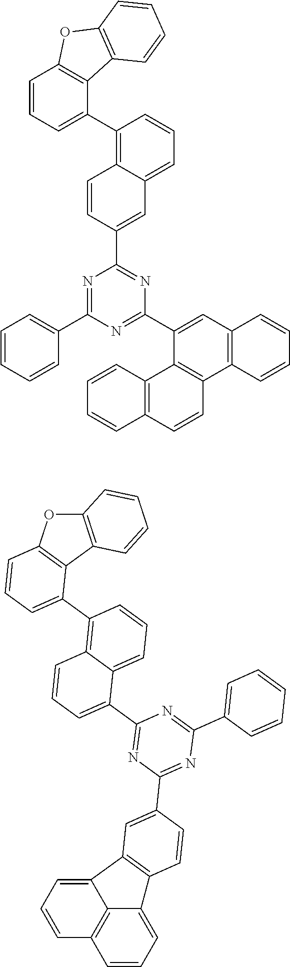 [SMILES: c1ccc(-c2nc(-c3ccc4c(-c5cccc6oc7ccccc7c56)cccc4c3)nc(-c3cc4ccccc4c4ccc5ccccc5c34)n2)cc1 ; c1ccc(-c2nc(-c3ccc4c(c3)-c3cccc5cccc-4c35)nc(-c3cccc4c(-c5cccc6oc7ccccc7c56)cccc34)n2)cc1]

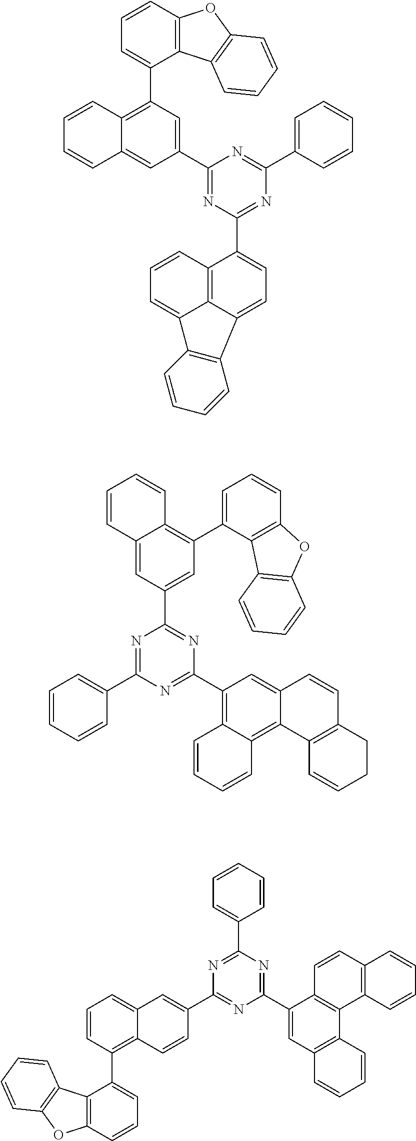 [SMILES: c1ccc(-c2nc(-c3cc(-c4cccc5oc6ccccc6c45)c4ccccc4c3)nc(-c3ccc4c5c(cccc35)-c3ccccc3-4)n2)cc1 ; C1=Cc2c(ccc3cc(-c4nc(-c5ccccc5)nc(-c5cc(-c6cccc7oc8ccccc8c67)c6ccccc6c5)n4)c4ccccc4c23)CC1 ; c1ccc(-c2nc(-c3ccc4c(-c5cccc6oc7ccccc7c56)cccc4c3)nc(-c3cc4ccccc4c4c3ccc3ccccc34)n2)cc1]

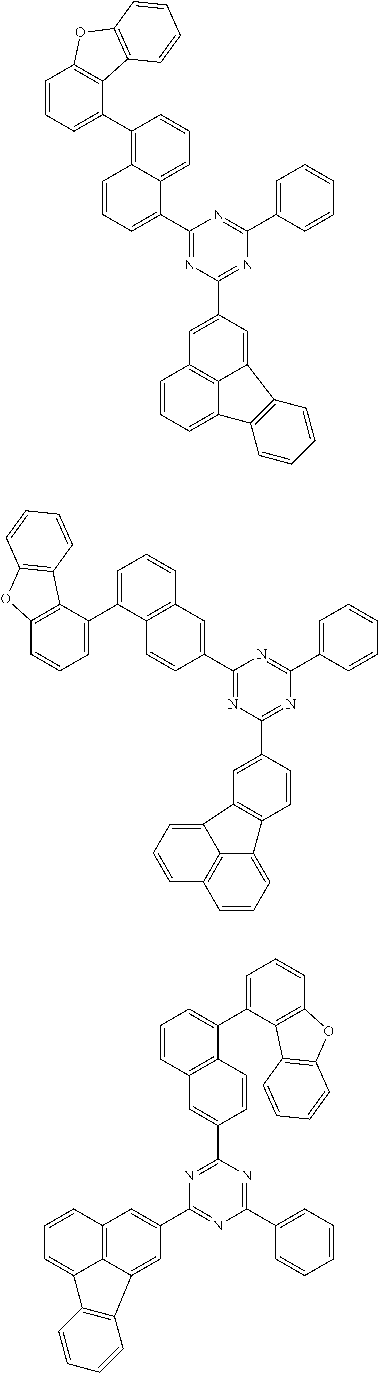 [SMILES: c1ccc(-c2nc(-c3cc4c5c(cccc5c3)-c3ccccc3-4)nc(-c3cccc4c(-c5cccc6oc7ccccc7c56)cccc34)n2)cc1 ; c1ccc(-c2nc(-c3ccc4c(c3)-c3cccc5cccc-4c35)nc(-c3ccc4c(-c5cccc6oc7ccccc7c56)cccc4c3)n2)cc1 ; c1ccc(-c2nc(-c3ccc4c(-c5cccc6oc7ccccc7c56)cccc4c3)nc(-c3cc4c5c(cccc5c3)-c3ccccc3-4)n2)cc1]

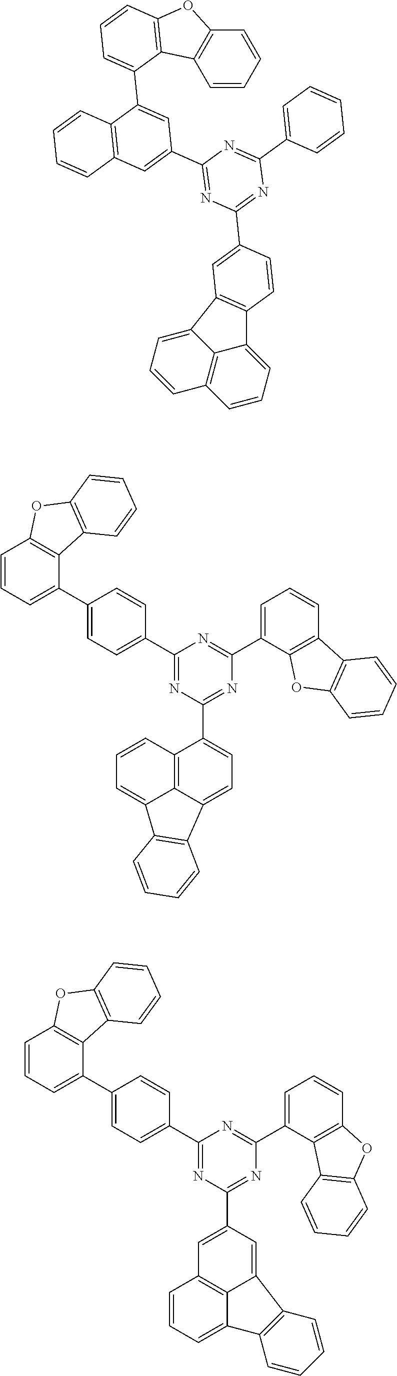 [SMILES: c1ccc(-c2nc(-c3ccc4c(c3)-c3cccc5cccc-4c35)nc(-c3cc(-c4cccc5oc6ccccc6c45)c4ccccc4c3)n2)cc1 ; c1ccc2c(c1)-c1cccc3c(-c4nc(-c5ccc(-c6cccc7oc8ccccc8c67)cc5)nc(-c5cccc6c5oc5ccccc56)n4)ccc-2c13 ; c1ccc2c(c1)-c1cccc3cc(-c4nc(-c5ccc(-c6cccc7oc8ccccc8c67)cc5)nc(-c5cccc6oc7ccccc7c56)n4)cc-2c13]

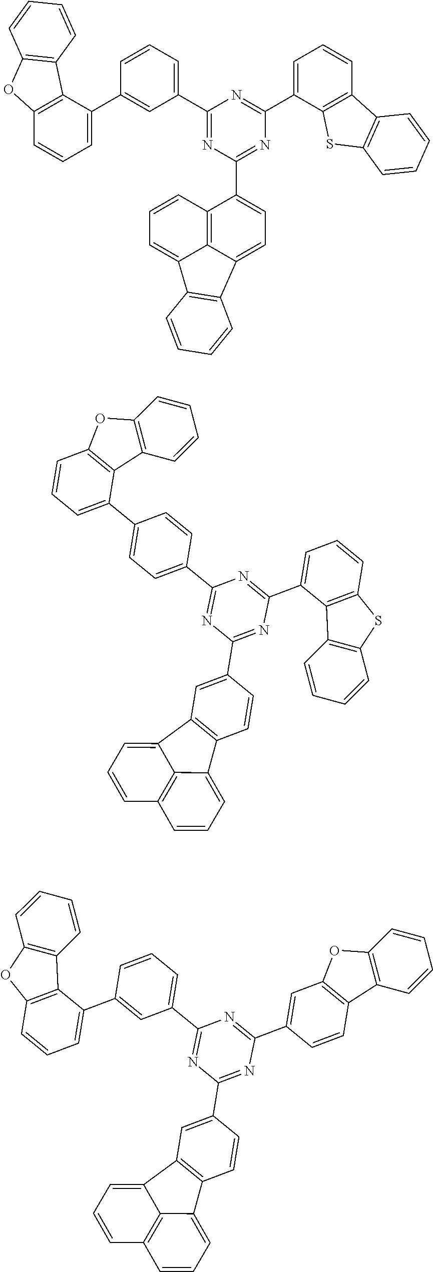 [SMILES: c1cc(-c2nc(-c3ccc4c5c(cccc35)-c3ccccc3-4)nc(-c3cccc4c3sc3ccccc34)n2)cc(-c2cccc3oc4ccccc4c23)c1 ; c1cc2c3c(cccc3c1)-c1cc(-c3nc(-c4ccc(-c5cccc6oc7ccccc7c56)cc4)nc(-c4cccc5sc6ccccc6c45)n3)ccc1-2 ; c1cc(-c2nc(-c3ccc4c(c3)-c3cccc5cccc-4c35)nc(-c3ccc4c(c3)oc3ccccc34)n2)cc(-c2cccc3oc4ccccc4c23)c1]

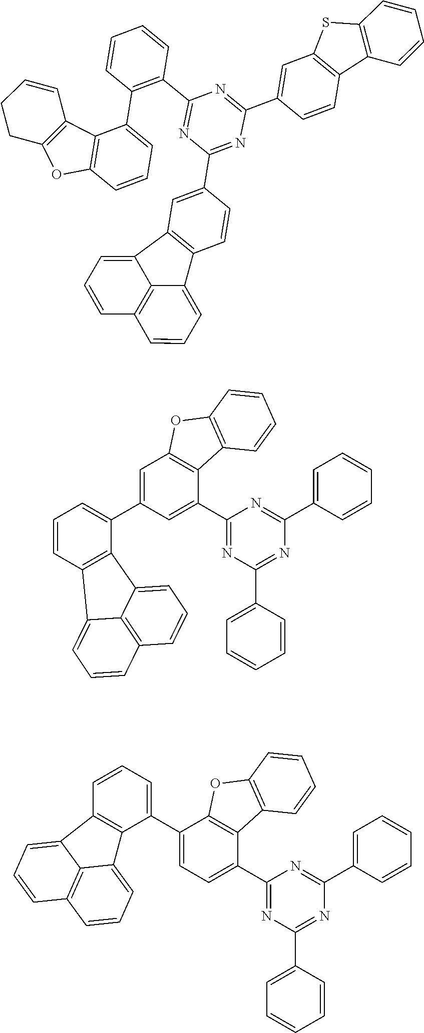 [SMILES: C1=Cc2c(oc3cccc(-c4ccccc4-c4nc(-c5ccc6c(c5)-c5cccc7cccc-6c57)nc(-c5ccc6c(c5)sc5ccccc56)n4)c23)CC1 ; c1ccc(-c2nc(-c3ccccc3)nc(-c3cc(-c4cccc5c4-c4cccc6cccc-5c46)cc4oc5ccccc5c34)n2)cc1 ; c1ccc(-c2nc(-c3ccccc3)nc(-c3ccc(-c4cccc5c4-c4cccc6cccc-5c46)c4oc5ccccc5c34)n2)cc1]

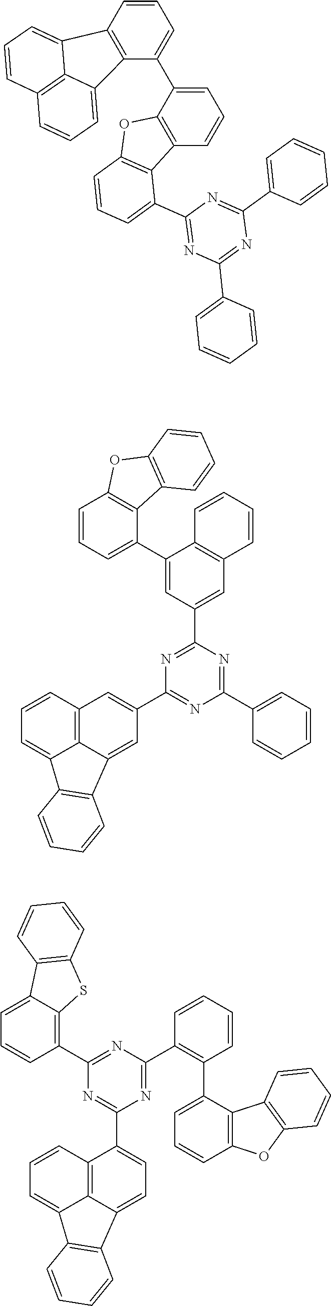 [SMILES: c1ccc(-c2nc(-c3ccccc3)nc(-c3cccc4oc5c(-c6cccc7c6-c6cccc8cccc-7c68)cccc5c34)n2)cc1 ; c1ccc(-c2nc(-c3cc(-c4cccc5oc6ccccc6c45)c4ccccc4c3)nc(-c3cc4c5c(cccc5c3)-c3ccccc3-4)n2)cc1 ; c1ccc(-c2cccc3oc4ccccc4c23)c(-c2nc(-c3ccc4c5c(cccc35)-c3ccccc3-4)nc(-c3cccc4c3sc3ccccc34)n2)c1]

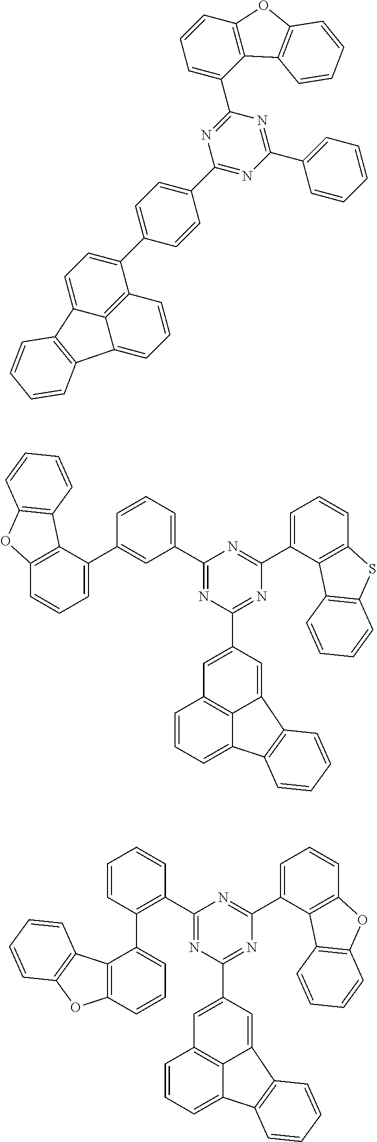 [SMILES: c1ccc(-c2nc(-c3ccc(-c4ccc5c6c(cccc46)-c4ccccc4-5)cc3)nc(-c3cccc4oc5ccccc5c34)n2)cc1 ; c1cc(-c2nc(-c3cc4c5c(cccc5c3)-c3ccccc3-4)nc(-c3cccc4sc5ccccc5c34)n2)cc(-c2cccc3oc4ccccc4c23)c1 ; c1ccc(-c2cccc3oc4ccccc4c23)c(-c2nc(-c3cc4c5c(cccc5c3)-c3ccccc3-4)nc(-c3cccc4oc5ccccc5c34)n2)c1]

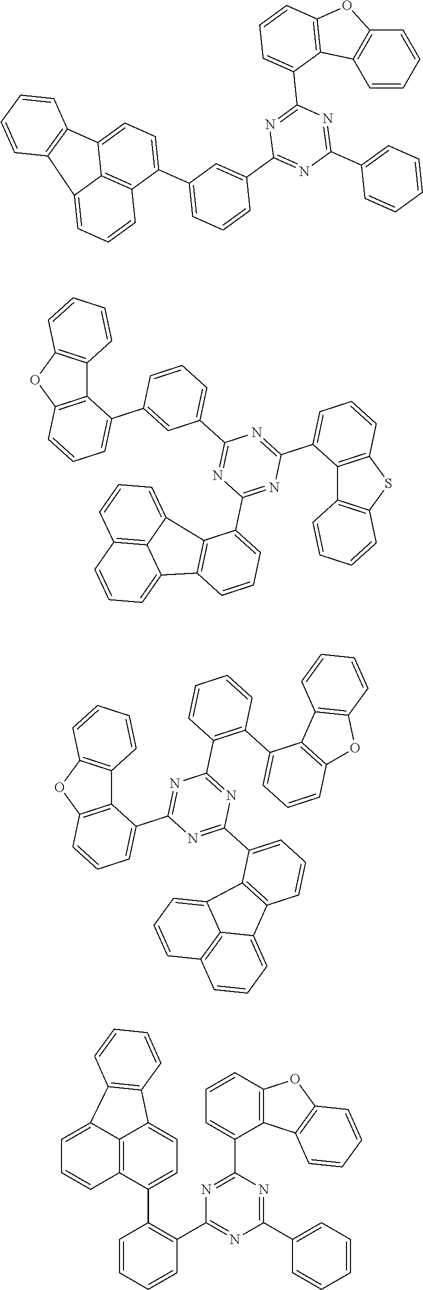 [SMILES: c1ccc(-c2nc(-c3cccc(-c4ccc5c6c(cccc46)-c4ccccc4-5)c3)nc(-c3cccc4oc5ccccc5c34)n2)cc1 ; c1cc(-c2nc(-c3cccc4c3-c3cccc5cccc-4c35)nc(-c3cccc4sc5ccccc5c34)n2)cc(-c2cccc3oc4ccccc4c23)c1 ; c1ccc(-c2cccc3oc4ccccc4c23)c(-c2nc(-c3cccc4c3-c3cccc5cccc-4c35)nc(-c3cccc4oc5ccccc5c34)n2)c1 ; c1ccc(-c2nc(-c3ccccc3-c3ccc4c5c(cccc35)-c3ccccc3-4)nc(-c3cccc4oc5ccccc5c34)n2)cc1]

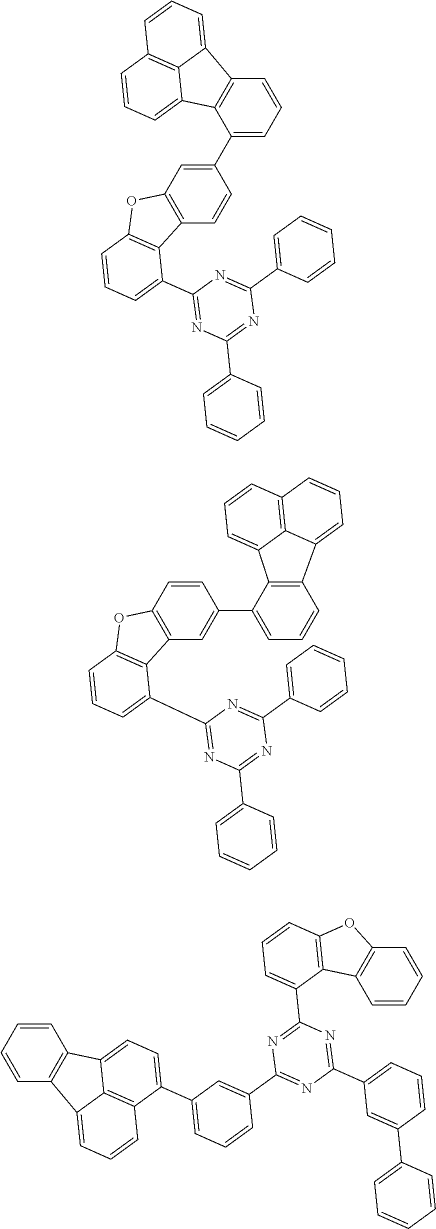

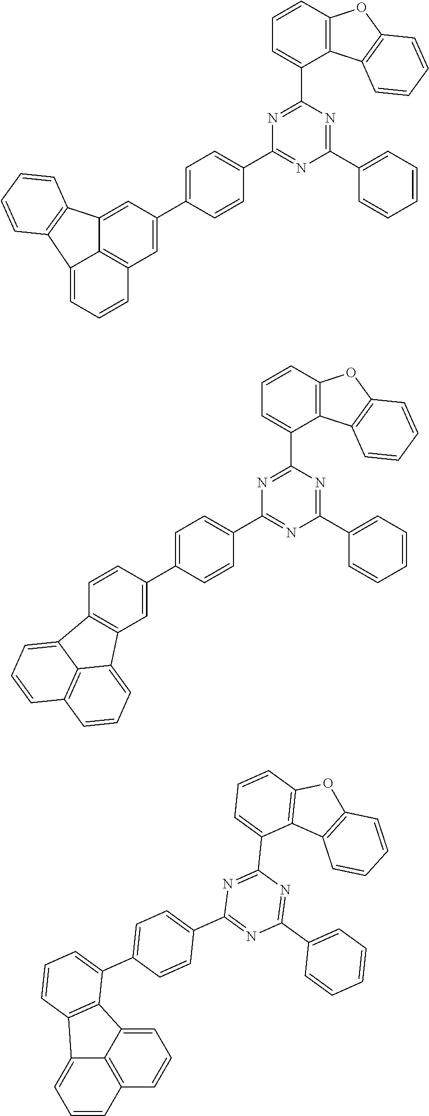 [SMILES: c1ccc(-c2nc(-c3ccc(-c4cc5c6c(cccc6c4)-c4ccccc4-5)cc3)nc(-c3cccc4oc5ccccc5c34)n2)cc1 ; c1ccc(-c2nc(-c3ccc(-c4ccc5c(c4)-c4cccc6cccc-5c46)cc3)nc(-c3cccc4oc5ccccc5c34)n2)cc1 ; c1ccc(-c2nc(-c3ccc(-c4cccc5c4-c4cccc6cccc-5c46)cc3)nc(-c3cccc4oc5ccccc5c34)n2)cc1]

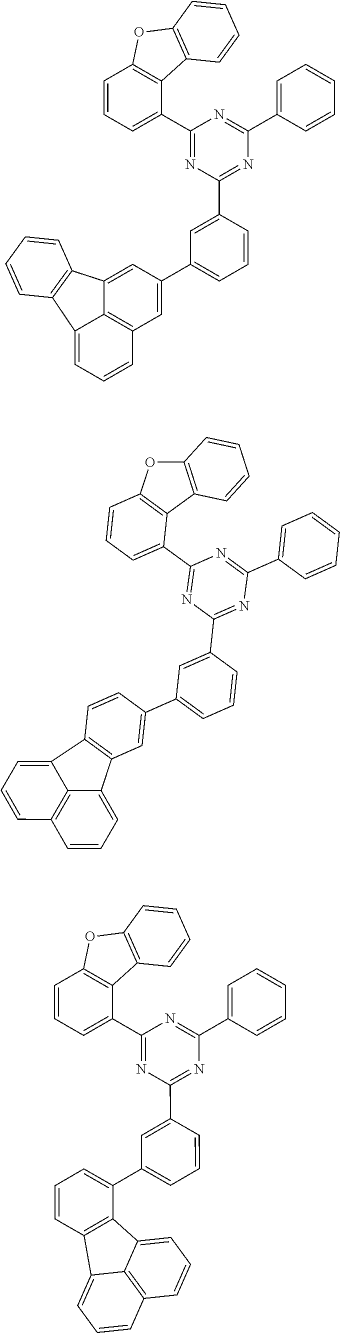 [SMILES: c1ccc(-c2nc(-c3cccc(-c4cc5c6c(cccc6c4)-c4ccccc4-5)c3)nc(-c3cccc4oc5ccccc5c34)n2)cc1 ; c1ccc(-c2nc(-c3cccc(-c4ccc5c(c4)-c4cccc6cccc-5c46)c3)nc(-c3cccc4oc5ccccc5c34)n2)cc1 ; c1ccc(-c2nc(-c3cccc(-c4cccc5c4-c4cccc6cccc-5c46)c3)nc(-c3cccc4oc5ccccc5c34)n2)cc1]

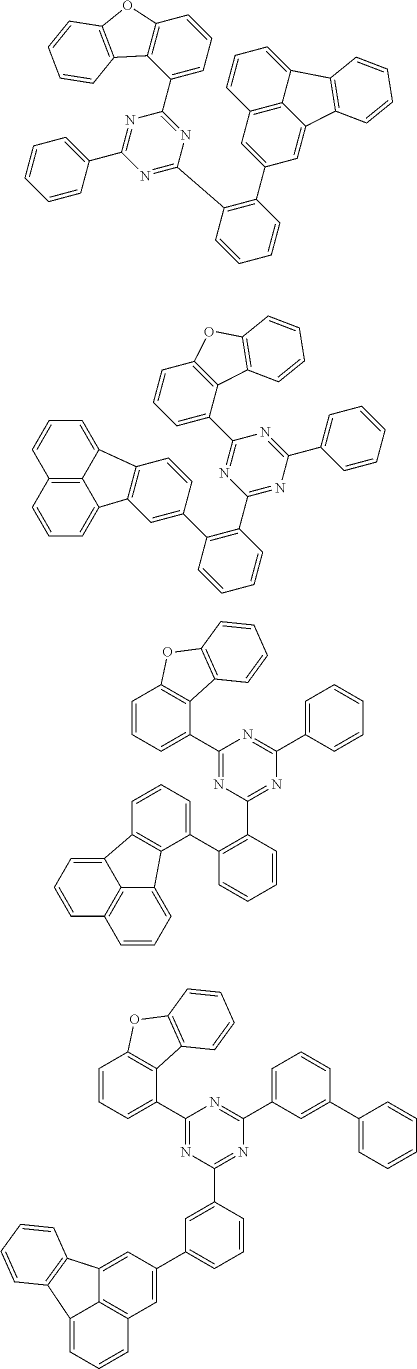

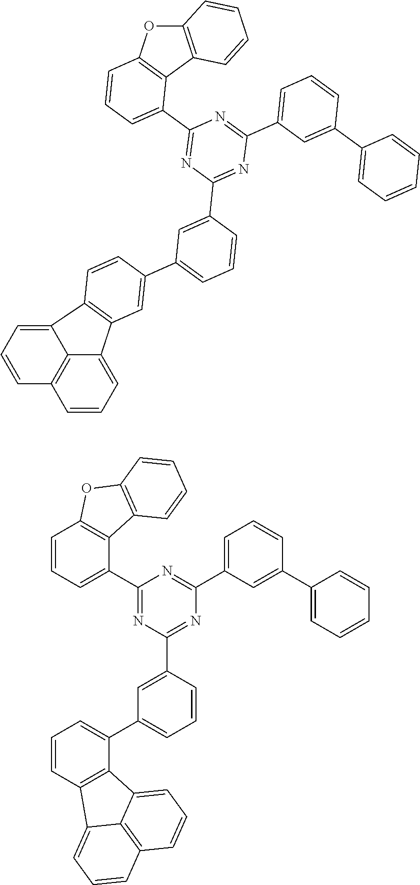 [SMILES: c1ccc(-c2cccc(-c3nc(-c4cccc(-c5ccc6c(c5)-c5cccc7cccc-6c57)c4)nc(-c4cccc5oc6ccccc6c45)n3)c2)cc1 ; c1ccc(-c2cccc(-c3nc(-c4cccc(-c5cccc6c5-c5cccc7cccc-6c57)c4)nc(-c4cccc5oc6ccccc6c45)n3)c2)cc1]

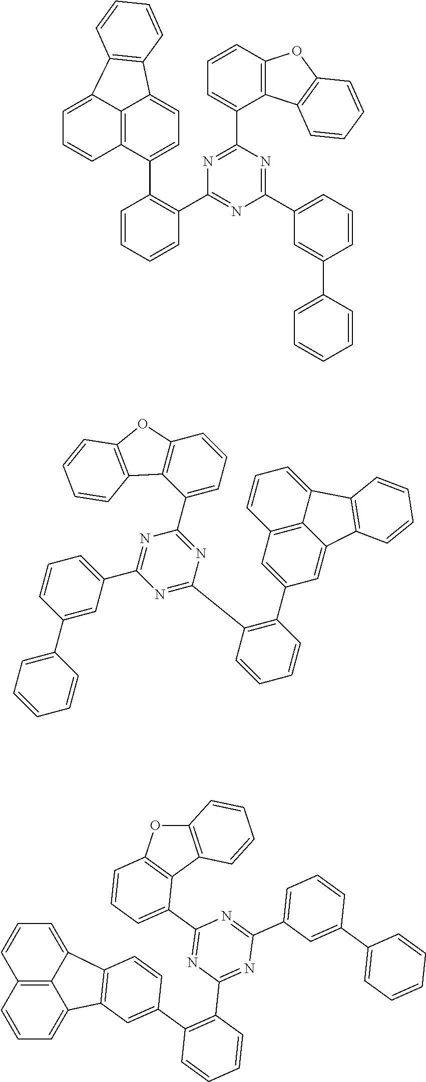 [SMILES: c1ccc(-c2cccc(-c3nc(-c4ccccc4-c4ccc5c6c(cccc46)-c4ccccc4-5)nc(-c4cccc5oc6ccccc6c45)n3)c2)cc1 ; c1ccc(-c2cccc(-c3nc(-c4ccccc4-c4cc5c6c(cccc6c4)-c4ccccc4-5)nc(-c4cccc5oc6ccccc6c45)n3)c2)cc1 ; c1ccc(-c2cccc(-c3nc(-c4ccccc4-c4ccc5c(c4)-c4cccc6cccc-5c46)nc(-c4cccc5oc6ccccc6c45)n3)c2)cc1]

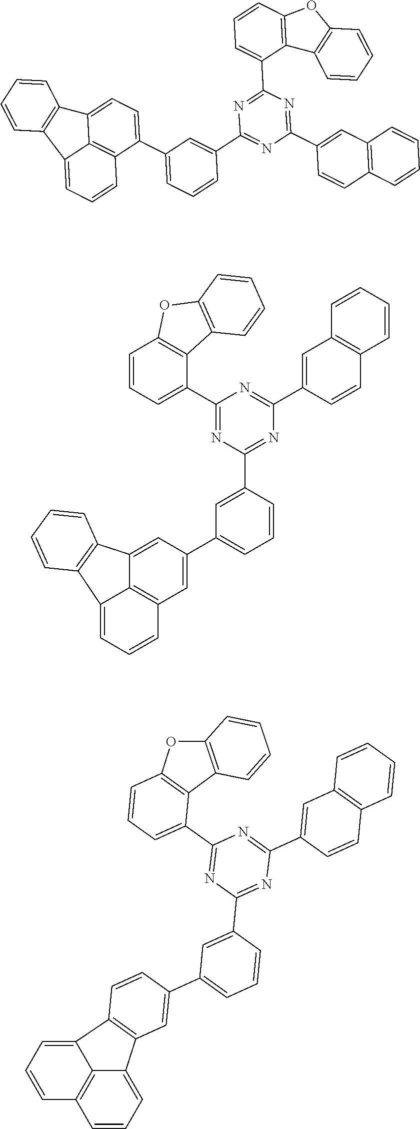 [SMILES: c1cc(-c2nc(-c3ccc4ccccc4c3)nc(-c3cccc4oc5ccccc5c34)n2)cc(-c2ccc3c4c(cccc24)-c2ccccc2-3)c1 ; c1cc(-c2cc3c4c(cccc4c2)-c2ccccc2-3)cc(-c2nc(-c3ccc4ccccc4c3)nc(-c3cccc4oc5ccccc5c34)n2)c1 ; c1cc(-c2ccc3c(c2)-c2cccc4cccc-3c24)cc(-c2nc(-c3ccc4ccccc4c3)nc(-c3cccc4oc5ccccc5c34)n2)c1]

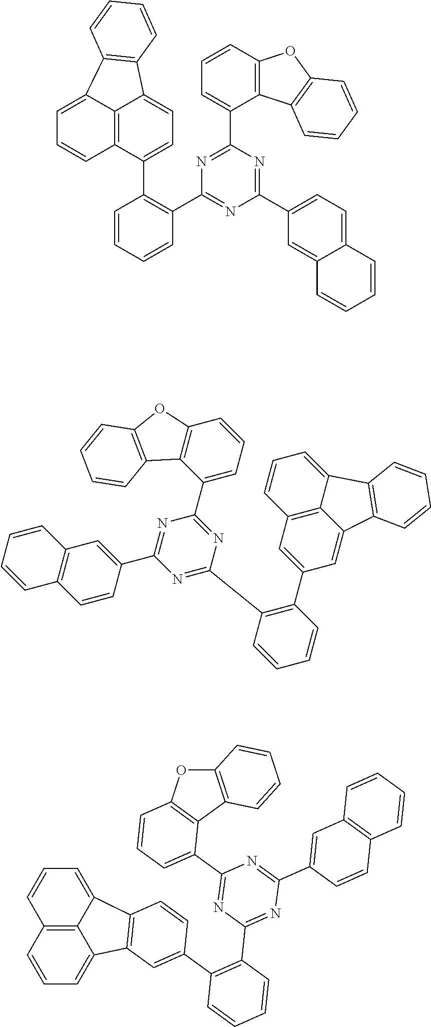 [SMILES: c1ccc(-c2ccc3c4c(cccc24)-c2ccccc2-3)c(-c2nc(-c3ccc4ccccc4c3)nc(-c3cccc4oc5ccccc5c34)n2)c1 ; c1ccc(-c2nc(-c3ccc4ccccc4c3)nc(-c3cccc4oc5ccccc5c34)n2)c(-c2cc3c4c(cccc4c2)-c2ccccc2-3)c1 ; c1ccc(-c2nc(-c3ccc4ccccc4c3)nc(-c3cccc4oc5ccccc5c34)n2)c(-c2ccc3c(c2)-c2cccc4cccc-3c24)c1]

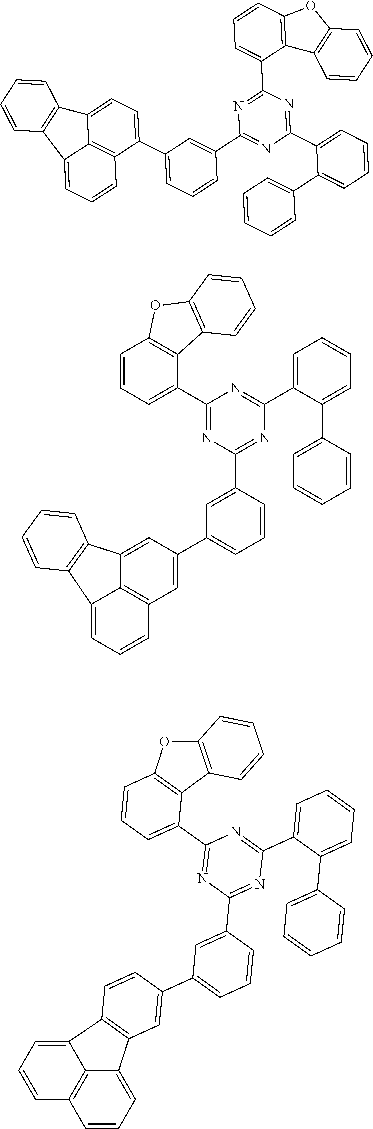 [SMILES: c1ccc(-c2ccccc2-c2nc(-c3cccc(-c4ccc5c6c(cccc46)-c4ccccc4-5)c3)nc(-c3cccc4oc5ccccc5c34)n2)cc1 ; c1ccc(-c2ccccc2-c2nc(-c3cccc(-c4cc5c6c(cccc6c4)-c4ccccc4-5)c3)nc(-c3cccc4oc5ccccc5c34)n2)cc1 ; c1ccc(-c2ccccc2-c2nc(-c3cccc(-c4ccc5c(c4)-c4cccc6cccc-5c46)c3)nc(-c3cccc4oc5ccccc5c34)n2)cc1]

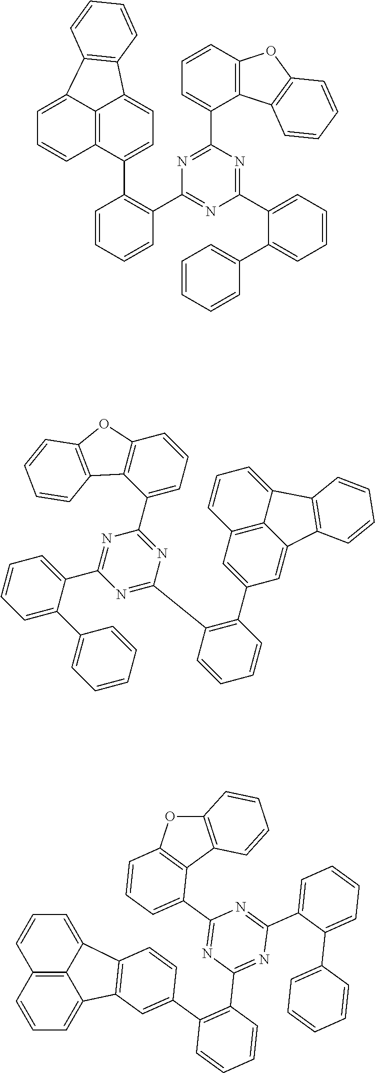 [SMILES: c1ccc(-c2ccccc2-c2nc(-c3ccccc3-c3ccc4c5c(cccc35)-c3ccccc3-4)nc(-c3cccc4oc5ccccc5c34)n2)cc1 ; c1ccc(-c2ccccc2-c2nc(-c3ccccc3-c3cc4c5c(cccc5c3)-c3ccccc3-4)nc(-c3cccc4oc5ccccc5c34)n2)cc1 ; c1ccc(-c2ccccc2-c2nc(-c3ccccc3-c3ccc4c(c3)-c3cccc5cccc-4c35)nc(-c3cccc4oc5ccccc5c34)n2)cc1]

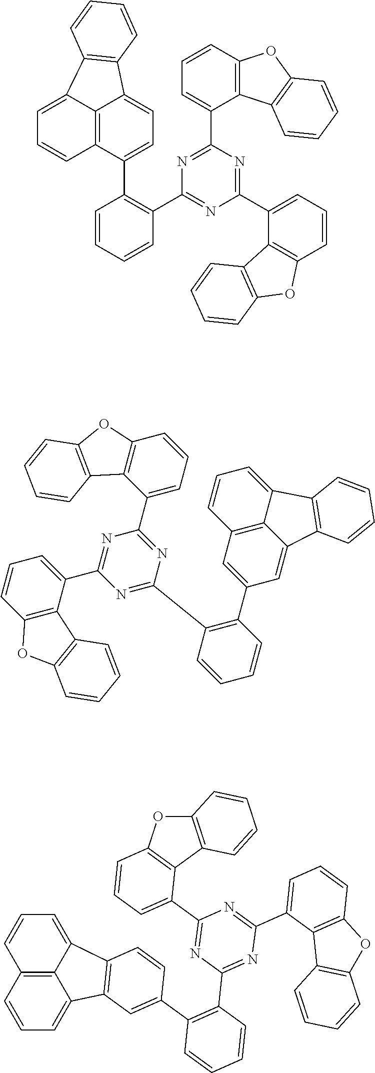 [SMILES: c1ccc(-c2ccc3c4c(cccc24)-c2ccccc2-3)c(-c2nc(-c3cccc4oc5ccccc5c34)nc(-c3cccc4oc5ccccc5c34)n2)c1 ; c1ccc(-c2nc(-c3cccc4oc5ccccc5c34)nc(-c3cccc4oc5ccccc5c34)n2)c(-c2cc3c4c(cccc4c2)-c2ccccc2-3)c1 ; c1ccc(-c2nc(-c3cccc4oc5ccccc5c34)nc(-c3cccc4oc5ccccc5c34)n2)c(-c2ccc3c(c2)-c2cccc4cccc-3c24)c1]

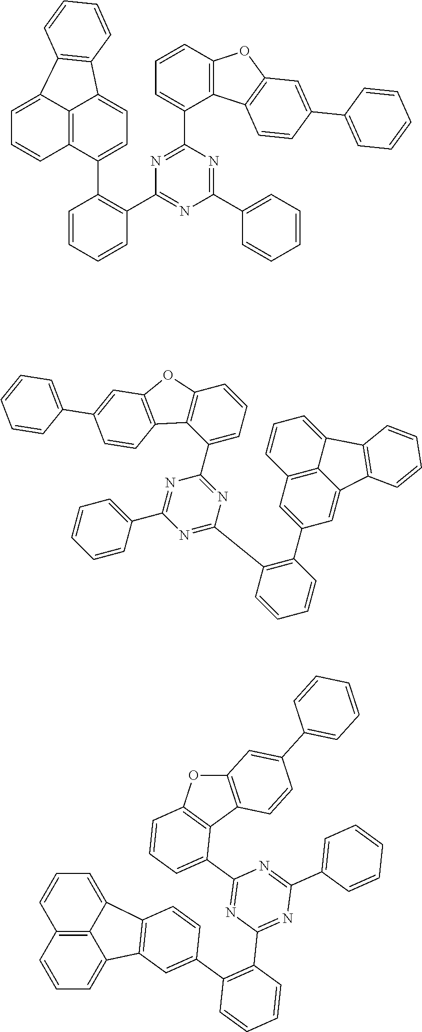 [SMILES: c1ccc(-c2ccc3c(c2)oc2cccc(-c4nc(-c5ccccc5)nc(-c5ccccc5-c5ccc6c7c(cccc57)-c5ccccc5-6)n4)c23)cc1 ; c1ccc(-c2ccc3c(c2)oc2cccc(-c4nc(-c5ccccc5)nc(-c5ccccc5-c5cc6c7c(cccc7c5)-c5ccccc5-6)n4)c23)cc1 ; c1ccc(-c2ccc3c(c2)oc2cccc(-c4nc(-c5ccccc5)nc(-c5ccccc5-c5ccc6c(c5)-c5cccc7cccc-6c57)n4)c23)cc1]

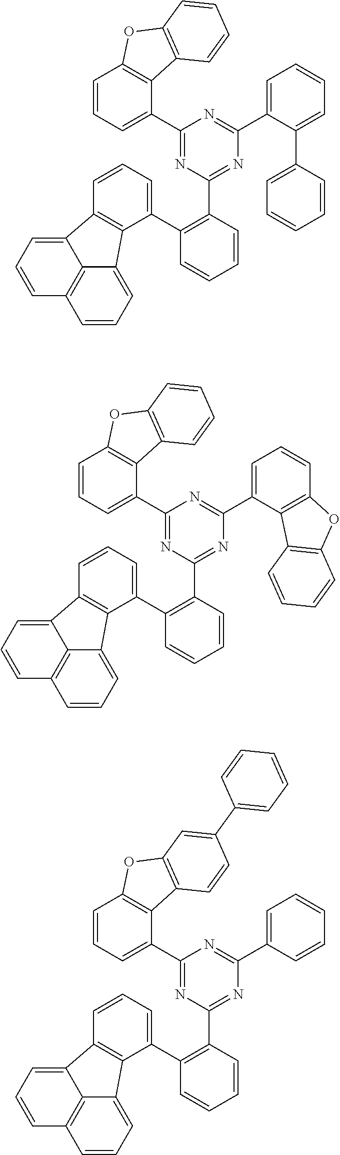 [SMILES: c1ccc(-c2ccccc2-c2nc(-c3ccccc3-c3cccc4c3-c3cccc5cccc-4c35)nc(-c3cccc4oc5ccccc5c34)n2)cc1 ; c1ccc(-c2cccc3c2-c2cccc4cccc-3c24)c(-c2nc(-c3cccc4oc5ccccc5c34)nc(-c3cccc4oc5ccccc5c34)n2)c1 ; c1ccc(-c2ccc3c(c2)oc2cccc(-c4nc(-c5ccccc5)nc(-c5ccccc5-c5cccc6c5-c5cccc7cccc-6c57)n4)c23)cc1]

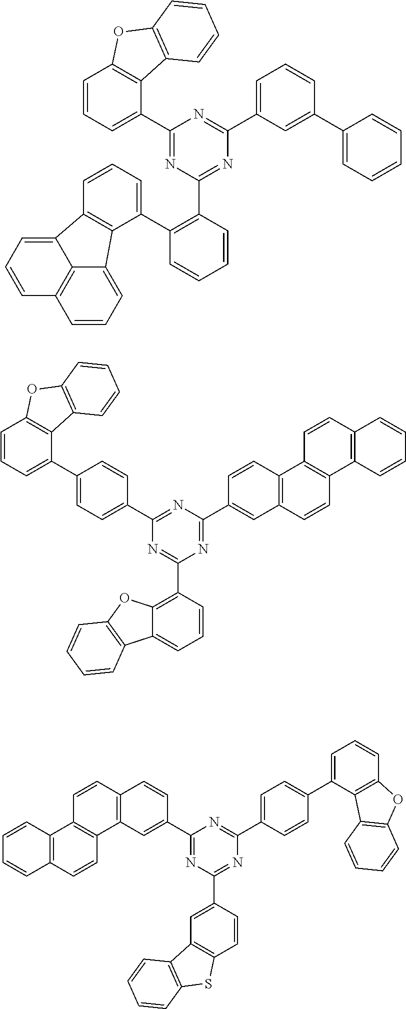

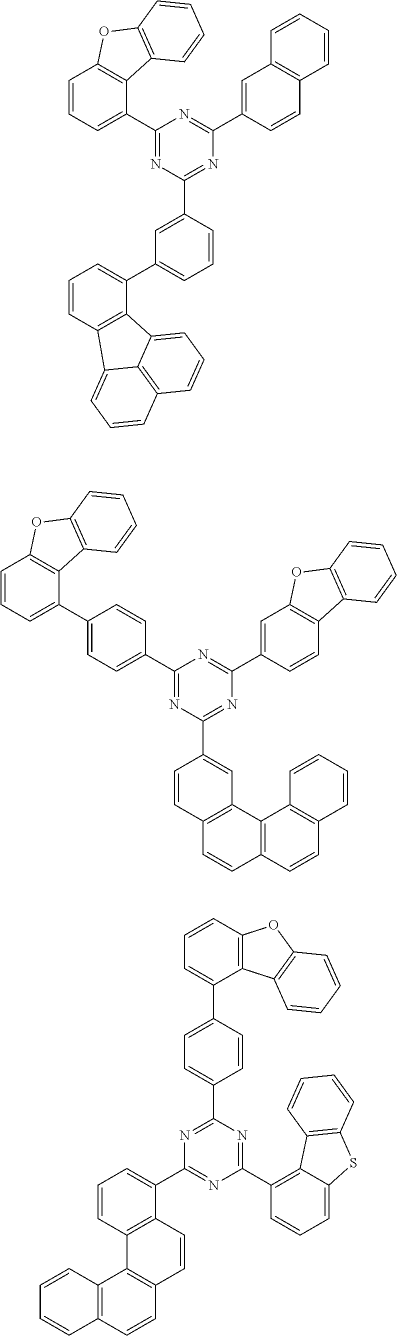 [SMILES: c1cc(-c2nc(-c3ccc4ccccc4c3)nc(-c3cccc4oc5ccccc5c34)n2)cc(-c2cccc3c2-c2cccc4cccc-3c24)c1 ; c1ccc2c(c1)ccc1ccc3ccc(-c4nc(-c5ccc(-c6cccc7oc8ccccc8c67)cc5)nc(-c5ccc6c(c5)oc5ccccc56)n4)cc3c12 ; c1ccc2c(c1)ccc1ccc3c(-c4nc(-c5ccc(-c6cccc7oc8ccccc8c67)cc5)nc(-c5cccc6sc7ccccc7c56)n4)cccc3c12]

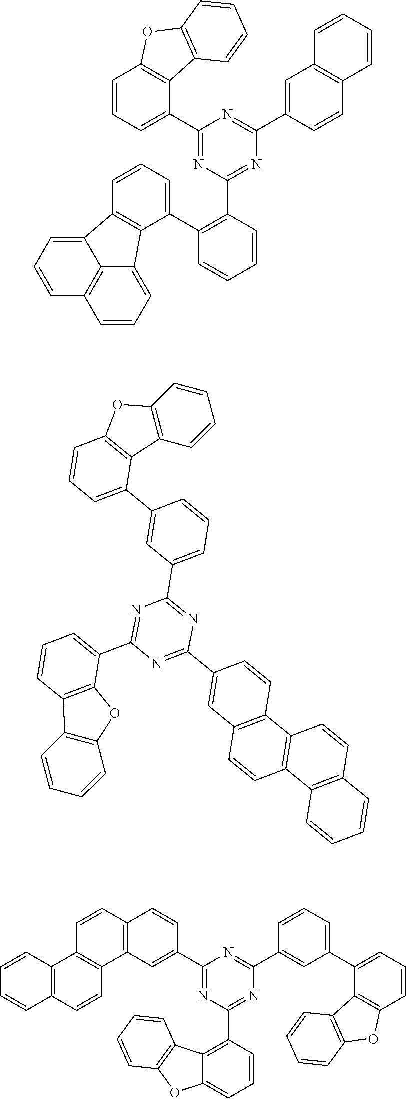 [SMILES: c1ccc(-c2cccc3c2-c2cccc4cccc-3c24)c(-c2nc(-c3ccc4ccccc4c3)nc(-c3cccc4oc5ccccc5c34)n2)c1 ; c1cc(-c2nc(-c3ccc4c(ccc5c6ccccc6ccc45)c3)nc(-c3cccc4c3oc3ccccc34)n2)cc(-c2cccc3oc4ccccc4c23)c1 ; c1cc(-c2nc(-c3ccc4ccc5c6ccccc6ccc5c4c3)nc(-c3cccc4oc5ccccc5c34)n2)cc(-c2cccc3oc4ccccc4c23)c1]

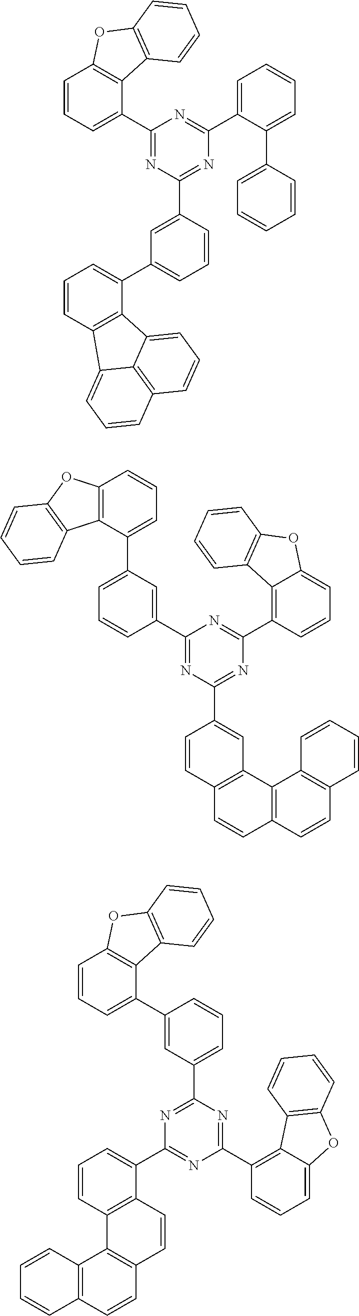 [SMILES: c1ccc(-c2ccccc2-c2nc(-c3cccc(-c4cccc5c4-c4cccc6cccc-5c46)c3)nc(-c3cccc4oc5ccccc5c34)n2)cc1 ; c1cc(-c2nc(-c3ccc4ccc5ccc6ccccc6c5c4c3)nc(-c3cccc4oc5ccccc5c34)n2)cc(-c2cccc3oc4ccccc4c23)c1 ; c1cc(-c2nc(-c3cccc4c3ccc3ccc5ccccc5c34)nc(-c3cccc4oc5ccccc5c34)n2)cc(-c2cccc3oc4ccccc4c23)c1]

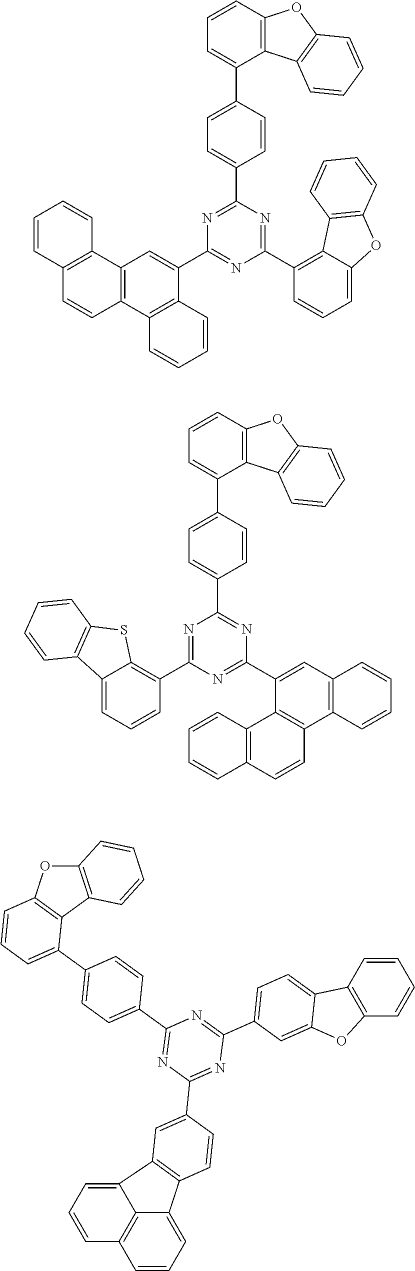 [SMILES: c1ccc2c(c1)ccc1c3ccccc3c(-c3nc(-c4ccc(-c5cccc6oc7ccccc7c56)cc4)nc(-c4cccc5oc6ccccc6c45)n3)cc21 ; c1ccc2c(c1)cc(-c1nc(-c3ccc(-c4cccc5oc6ccccc6c45)cc3)nc(-c3cccc4c3sc3ccccc34)n1)c1c3ccccc3ccc21 ; c1cc2c3c(cccc3c1)-c1cc(-c3nc(-c4ccc(-c5cccc6oc7ccccc7c56)cc4)nc(-c4ccc5c(c4)oc4ccccc45)n3)ccc1-2]

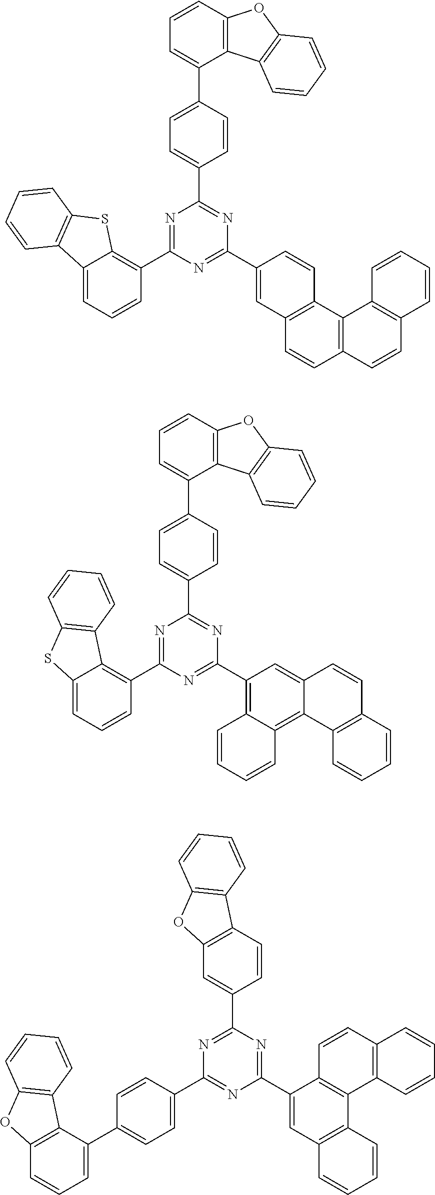 [SMILES: c1ccc2c(c1)ccc1ccc3cc(-c4nc(-c5ccc(-c6cccc7oc8ccccc8c67)cc5)nc(-c5cccc6c5sc5ccccc56)n4)ccc3c12 ; c1ccc2c(c1)ccc1cc(-c3nc(-c4ccc(-c5cccc6oc7ccccc7c56)cc4)nc(-c4cccc5sc6ccccc6c45)n3)c3ccccc3c12 ; c1ccc2c(c1)ccc1c(-c3nc(-c4ccc(-c5cccc6oc7ccccc7c56)cc4)nc(-c4ccc5c(c4)oc4ccccc45)n3)cc3ccccc3c12]

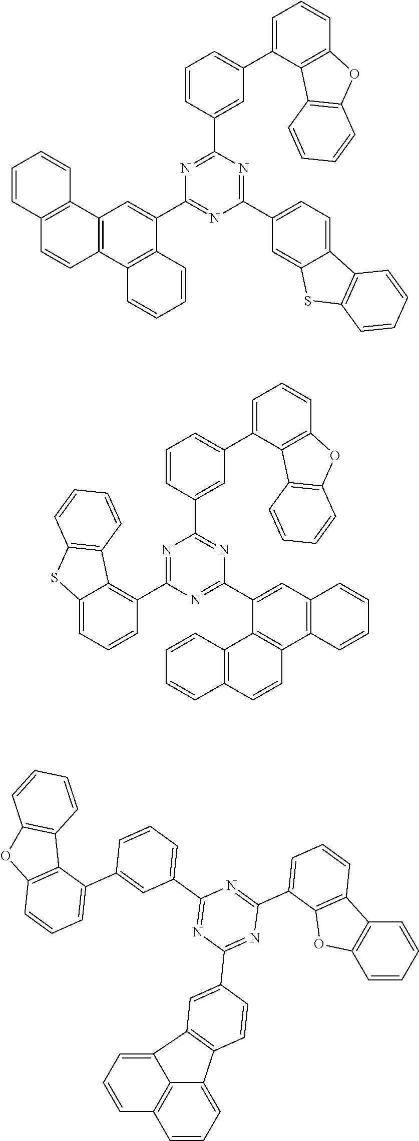 [SMILES: c1cc(-c2nc(-c3ccc4c(c3)sc3ccccc34)nc(-c3cc4c5ccccc5ccc4c4ccccc34)n2)cc(-c2cccc3oc4ccccc4c23)c1 ; c1cc(-c2nc(-c3cccc4sc5ccccc5c34)nc(-c3cc4ccccc4c4ccc5ccccc5c34)n2)cc(-c2cccc3oc4ccccc4c23)c1 ; c1cc(-c2nc(-c3ccc4c(c3)-c3cccc5cccc-4c35)nc(-c3cccc4c3oc3ccccc34)n2)cc(-c2cccc3oc4ccccc4c23)c1]

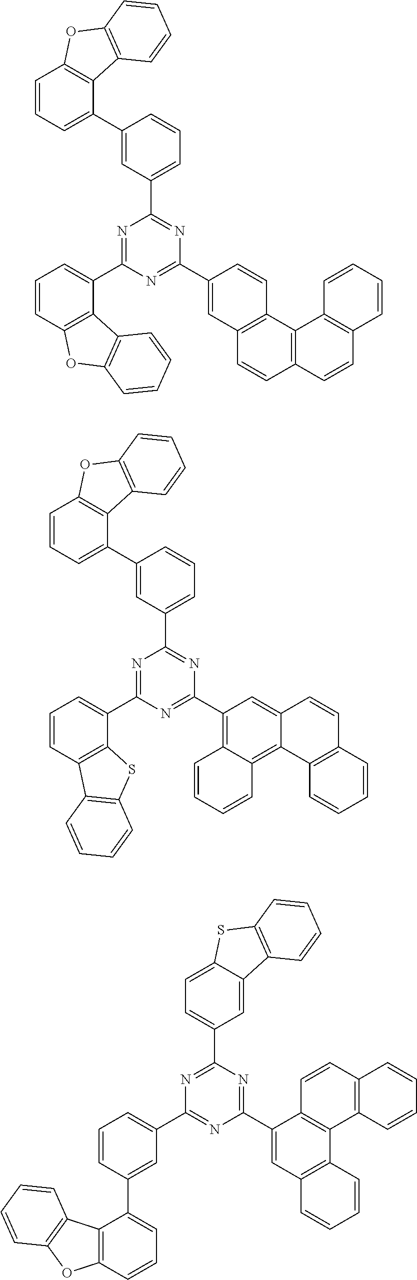

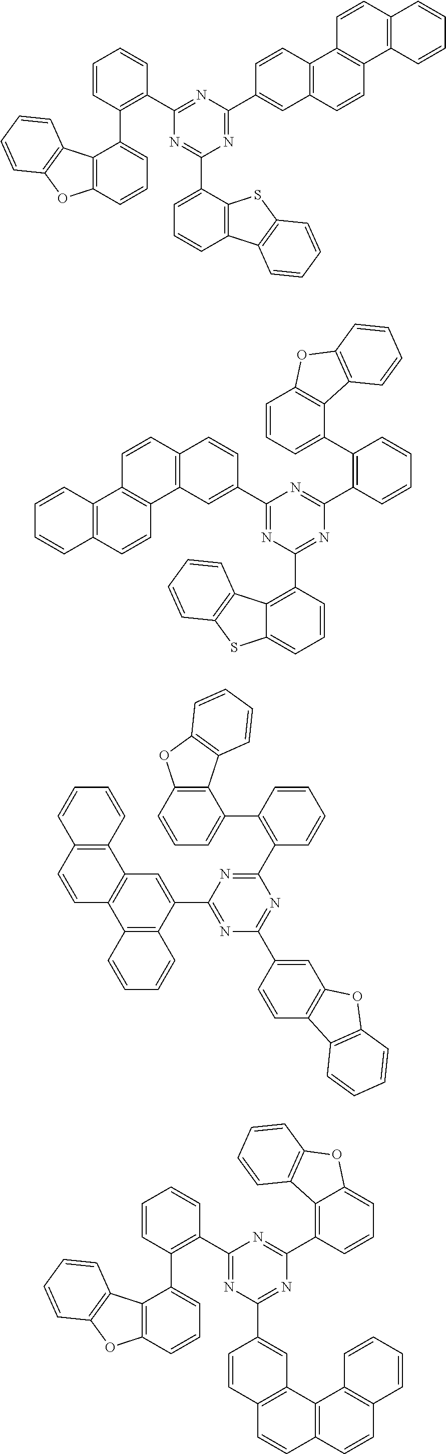

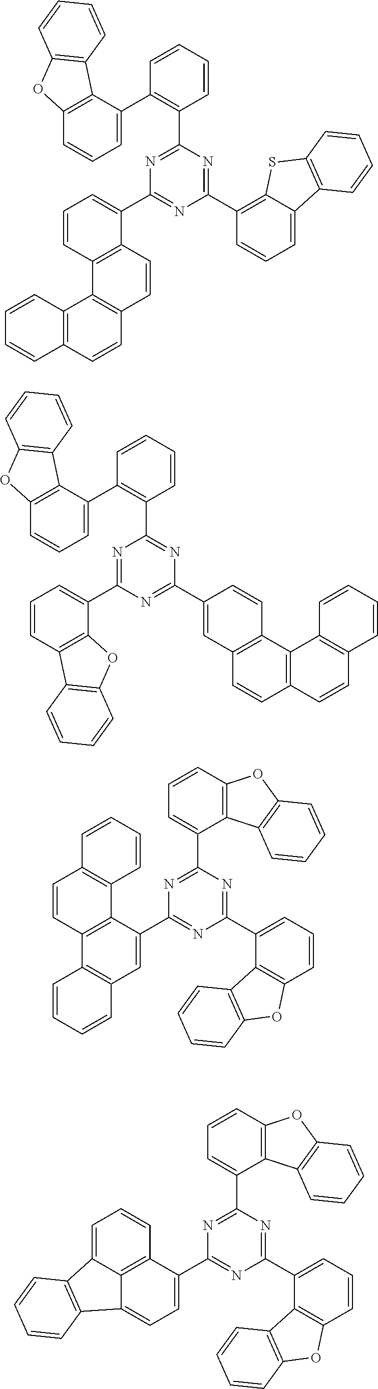

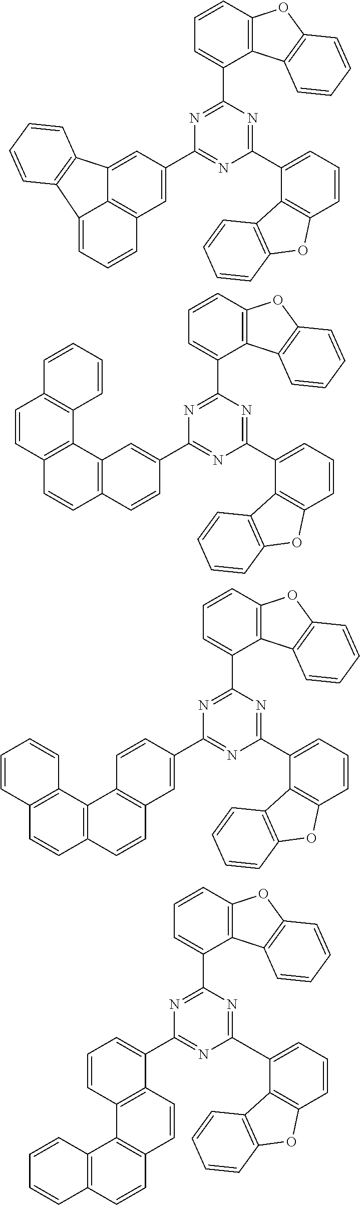

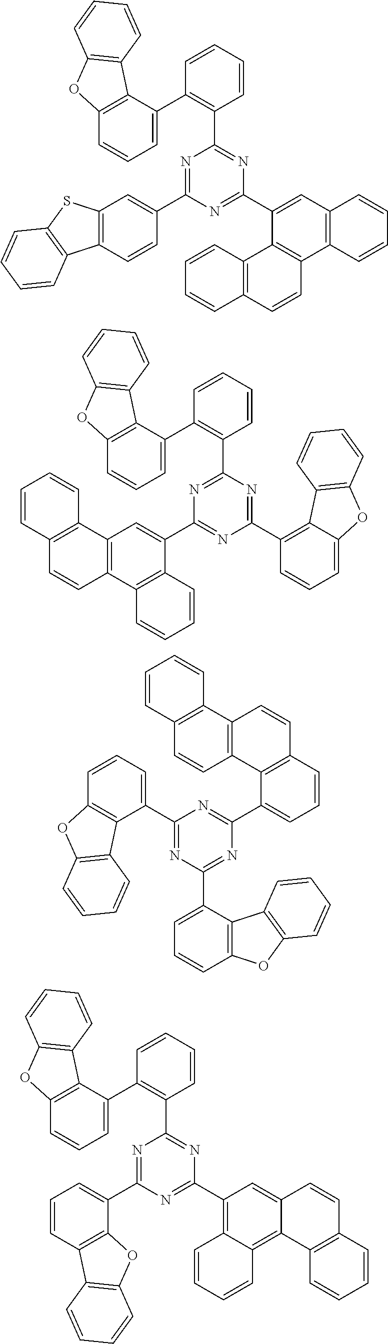

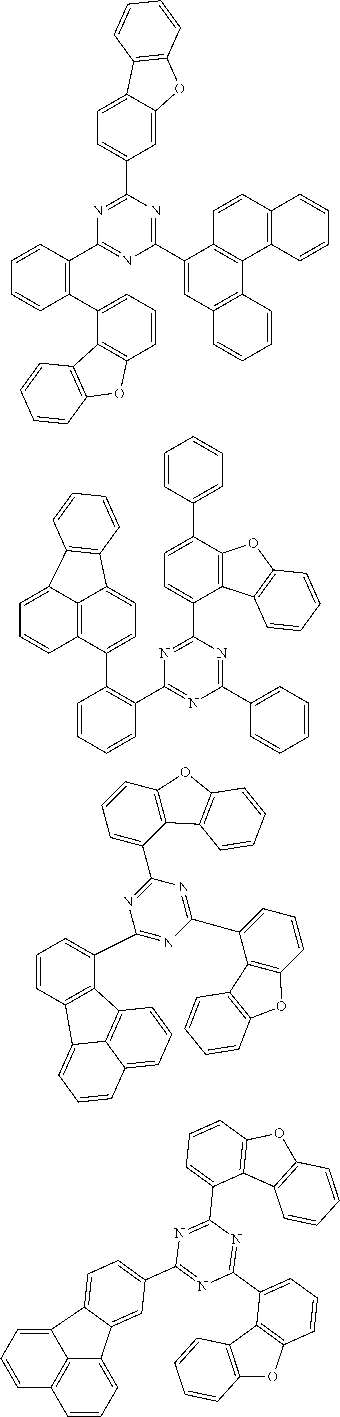

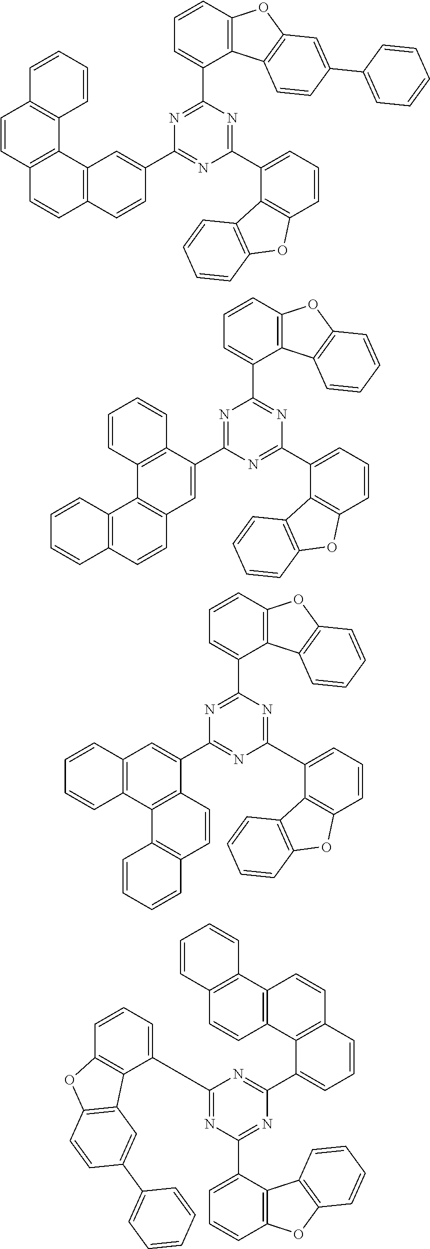

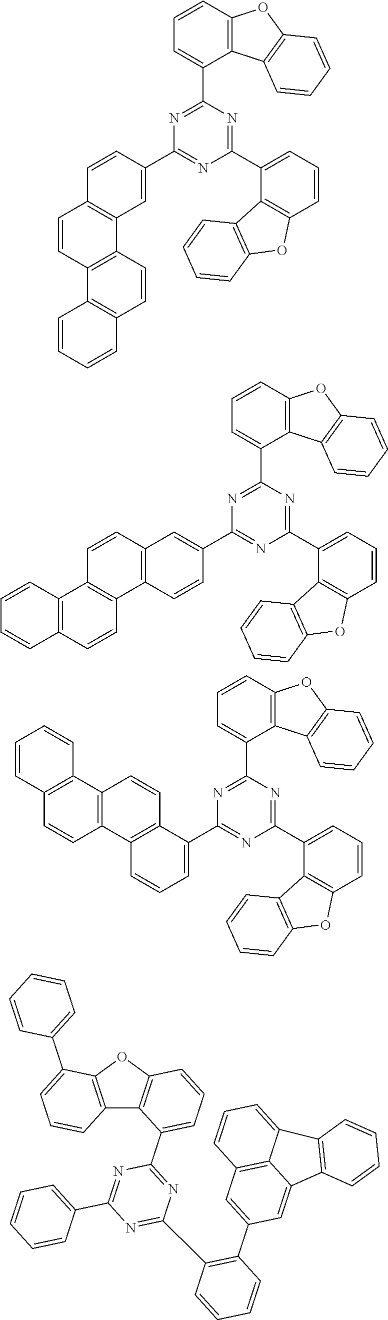

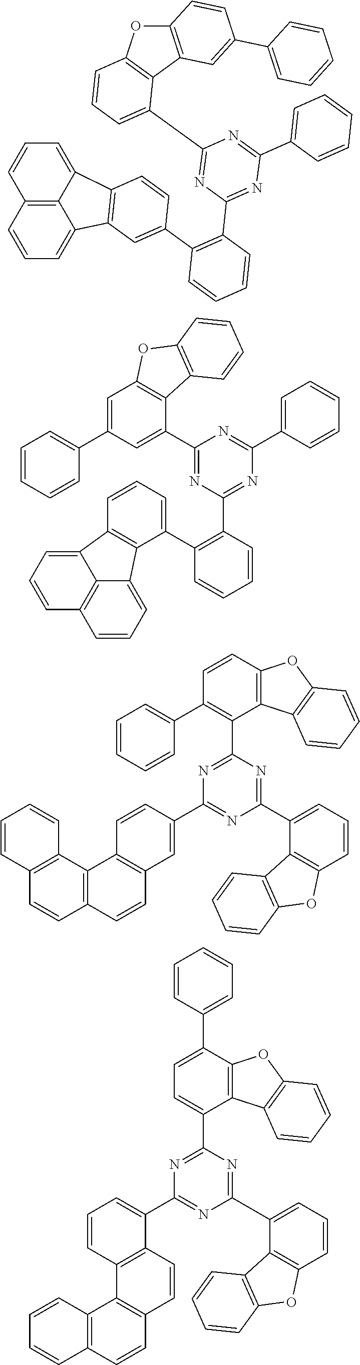

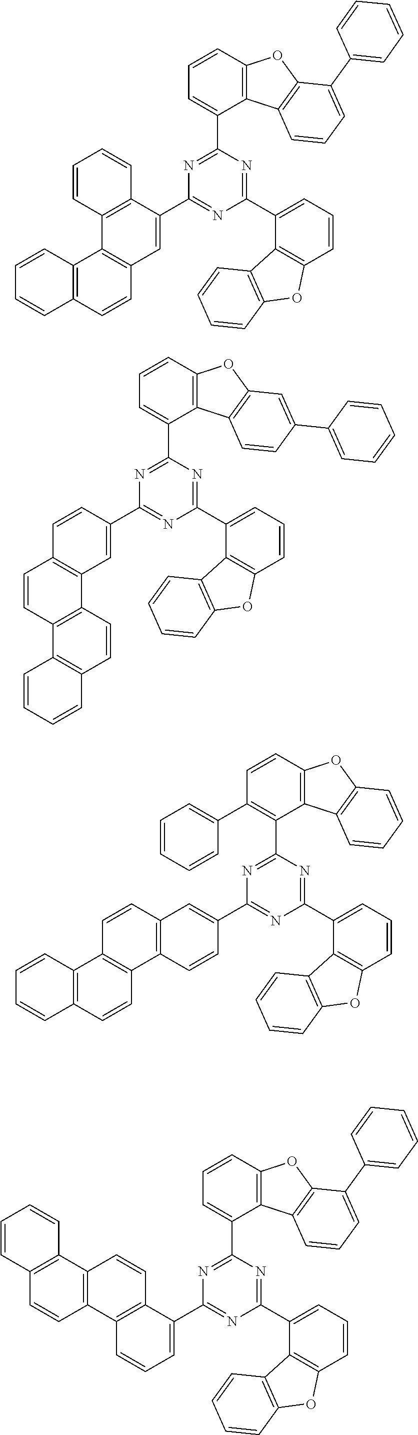

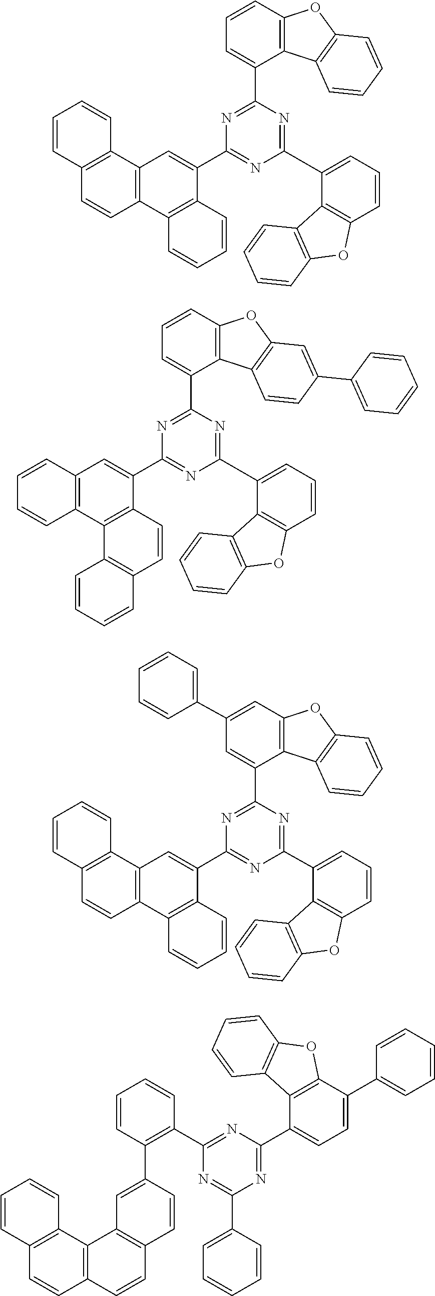

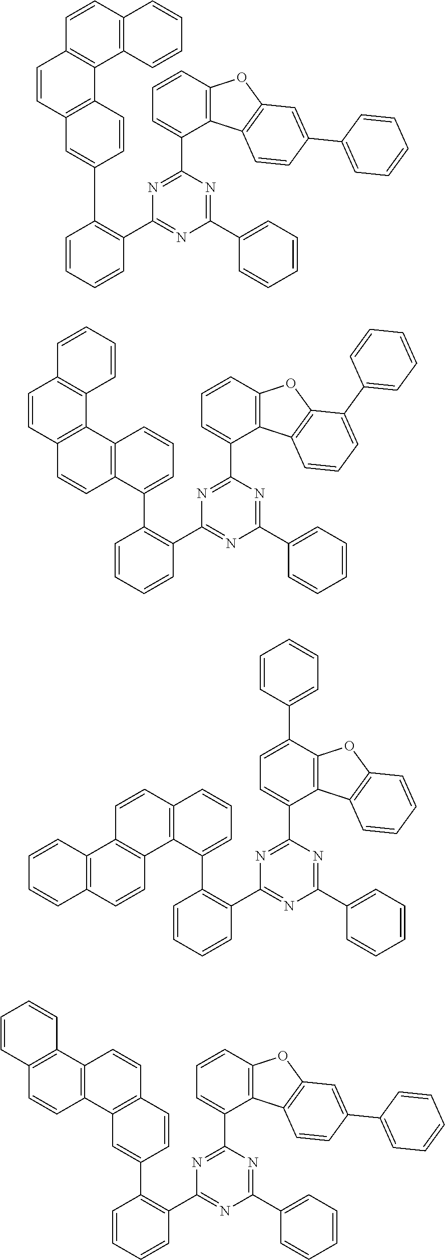

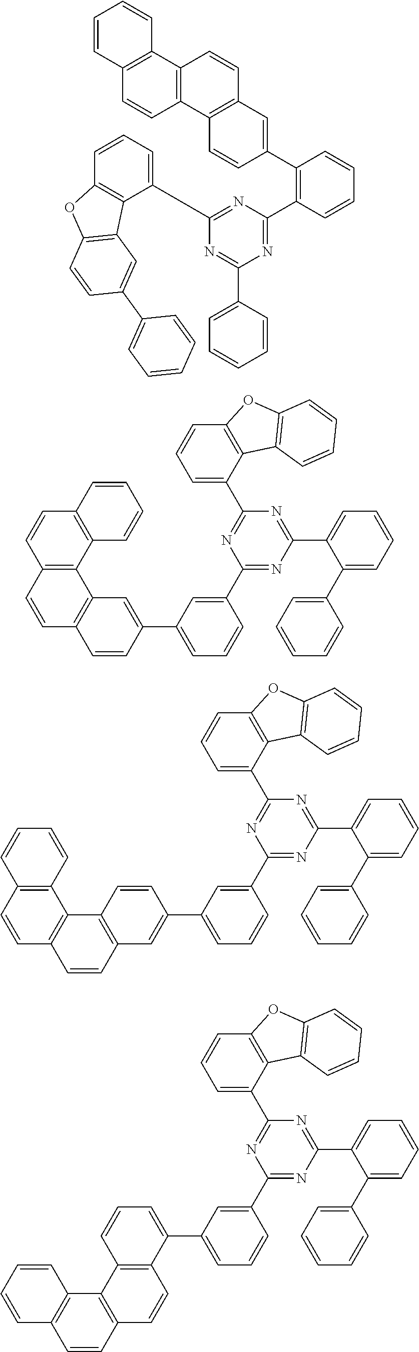

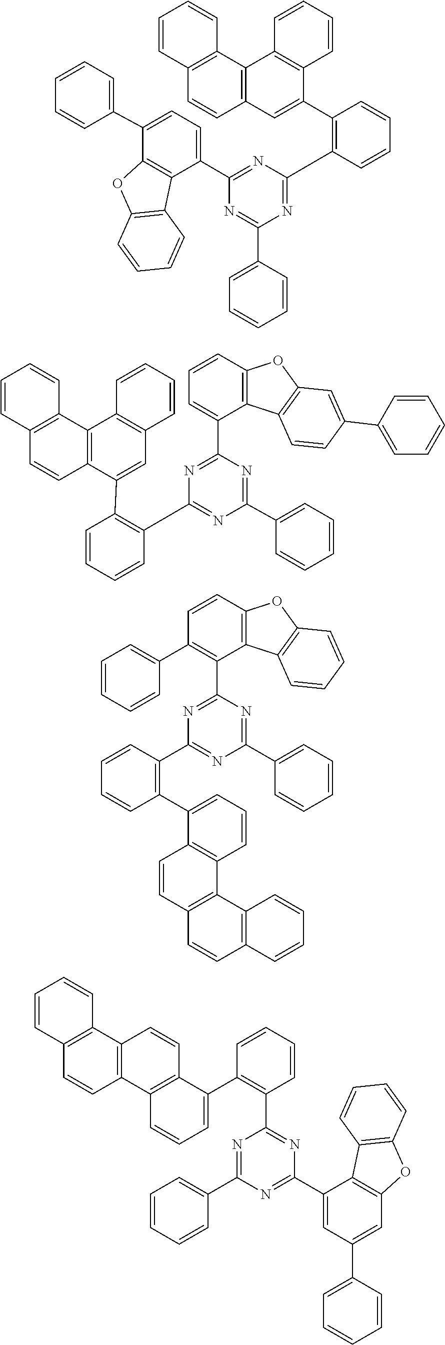

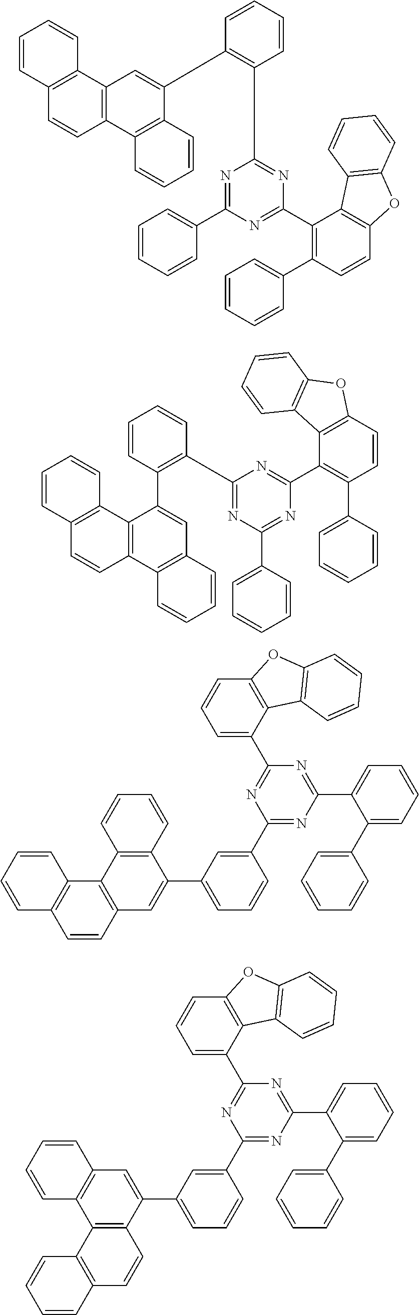

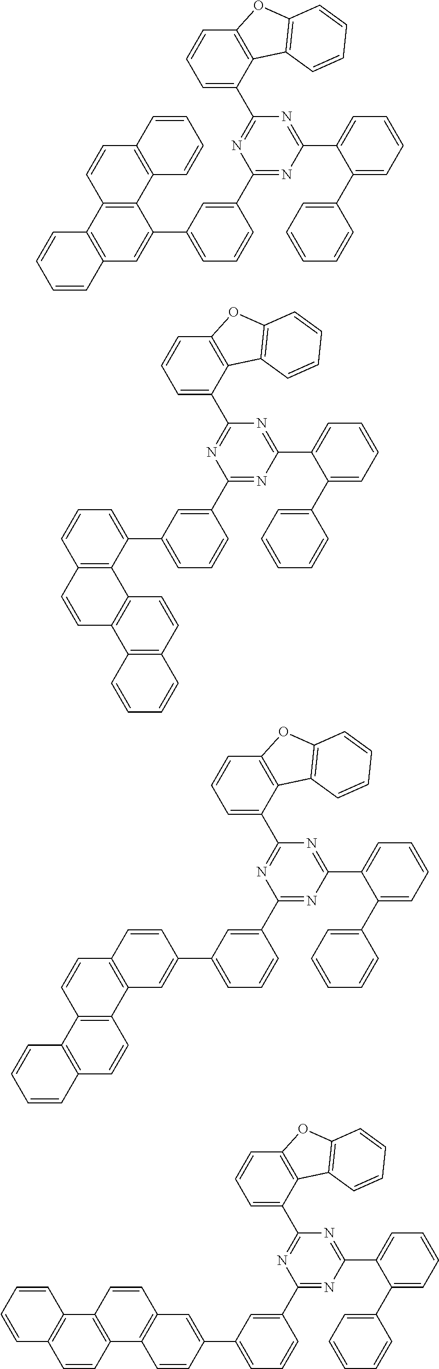 [SMILES: c1ccc(-c2ccccc2-c2nc(-c3cccc(-c4cc5ccccc5c5ccc6ccccc6c45)c3)nc(-c3cccc4oc5ccccc5c34)n2)cc1 ; c1ccc(-c2ccccc2-c2nc(-c3cccc(-c4cccc5ccc6c7ccccc7ccc6c45)c3)nc(-c3cccc4oc5ccccc5c34)n2)cc1 ; c1ccc(-c2ccccc2-c2nc(-c3cccc(-c4ccc5ccc6c7ccccc7ccc6c5c4)c3)nc(-c3cccc4oc5ccccc5c34)n2)cc1 ; c1ccc(-c2ccccc2-c2nc(-c3cccc(-c4ccc5c(ccc6c7ccccc7ccc56)c4)c3)nc(-c3cccc4oc5ccccc5c34)n2)cc1]

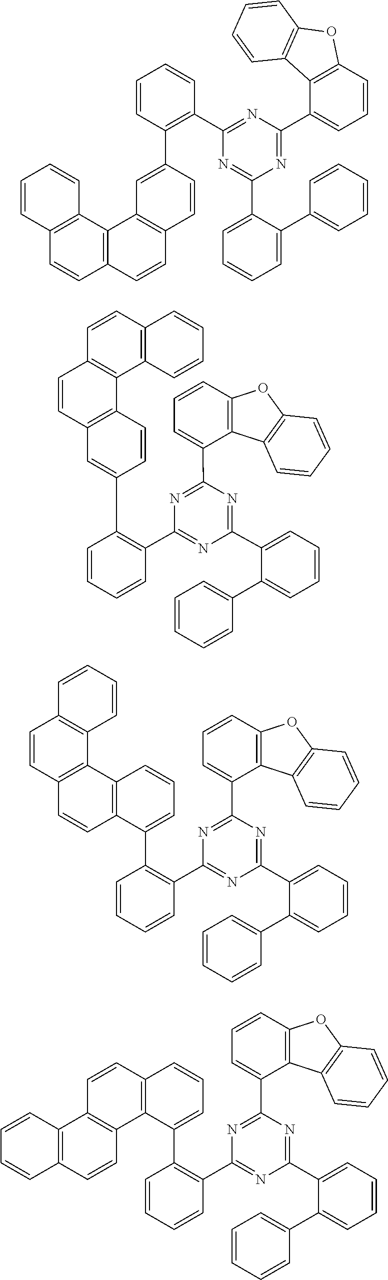

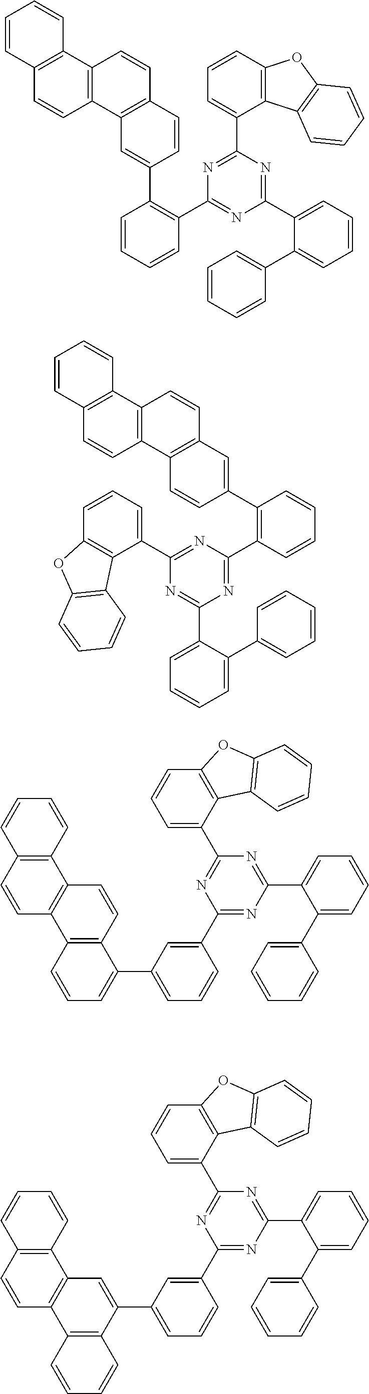

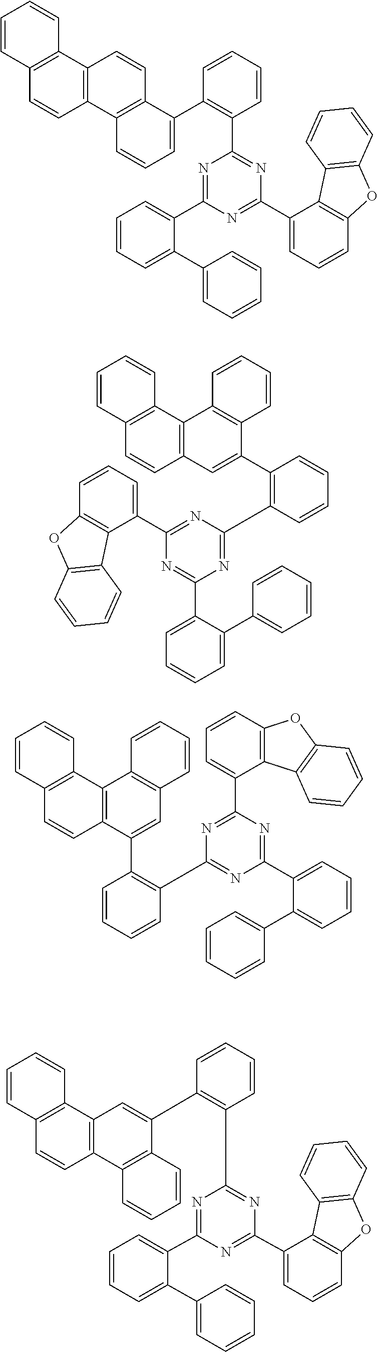

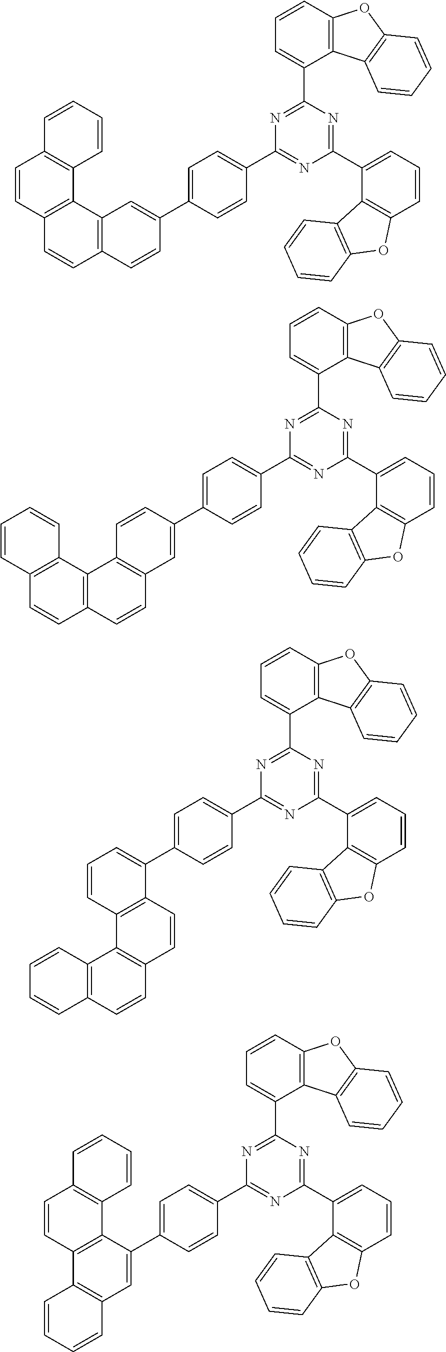

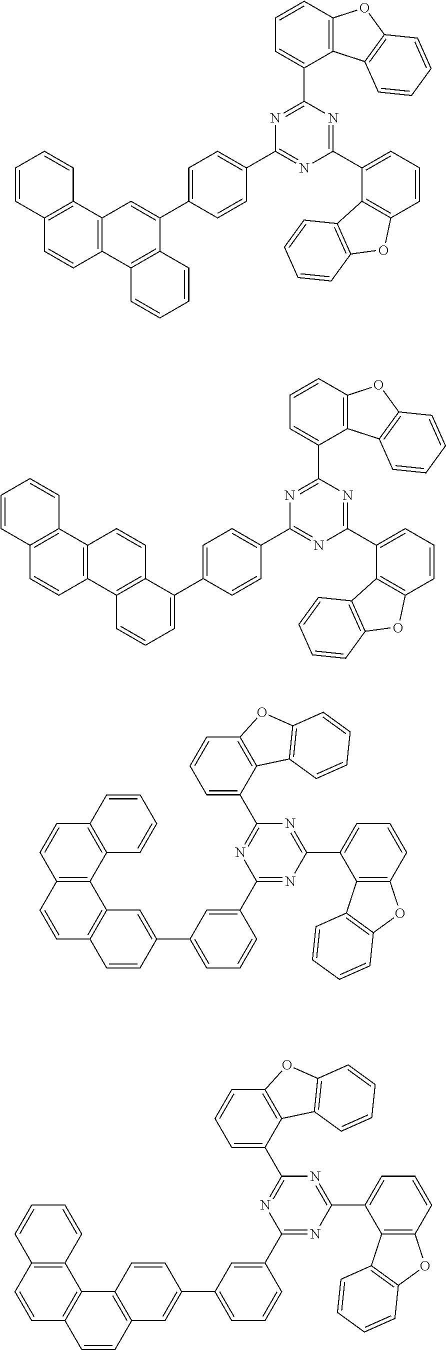 [SMILES: c1ccc2c(c1)ccc1c3ccccc3c(-c3ccc(-c4nc(-c5cccc6oc7ccccc7c56)nc(-c5cccc6oc7ccccc7c56)n4)cc3)cc21 ; c1ccc2c(c1)ccc1c3cccc(-c4ccc(-c5nc(-c6cccc7oc8ccccc8c67)nc(-c6cccc7oc8ccccc8c67)n5)cc4)c3ccc21 ; c1cc(-c2ccc3ccc4ccc5ccccc5c4c3c2)cc(-c2nc(-c3cccc4oc5ccccc5c34)nc(-c3cccc4oc5ccccc5c34)n2)c1 ; c1cc(-c2ccc3c(ccc4ccc5ccccc5c43)c2)cc(-c2nc(-c3cccc4oc5ccccc5c34)nc(-c3cccc4oc5ccccc5c34)n2)c1]

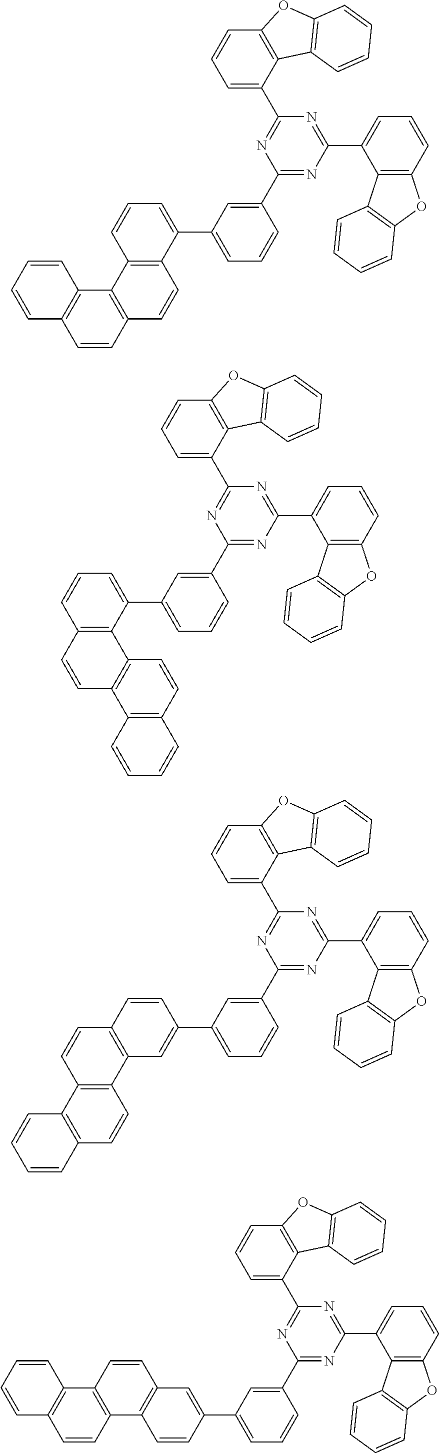 [SMILES: c1cc(-c2nc(-c3cccc4oc5ccccc5c34)nc(-c3cccc4oc5ccccc5c34)n2)cc(-c2cccc3c2ccc2ccc4ccccc4c23)c1 ; c1cc(-c2nc(-c3cccc4oc5ccccc5c34)nc(-c3cccc4oc5ccccc5c34)n2)cc(-c2cccc3ccc4c5ccccc5ccc4c23)c1 ; c1cc(-c2ccc3ccc4c5ccccc5ccc4c3c2)cc(-c2nc(-c3cccc4oc5ccccc5c34)nc(-c3cccc4oc5ccccc5c34)n2)c1 ; c1cc(-c2ccc3c(ccc4c5ccccc5ccc34)c2)cc(-c2nc(-c3cccc4oc5ccccc5c34)nc(-c3cccc4oc5ccccc5c34)n2)c1]

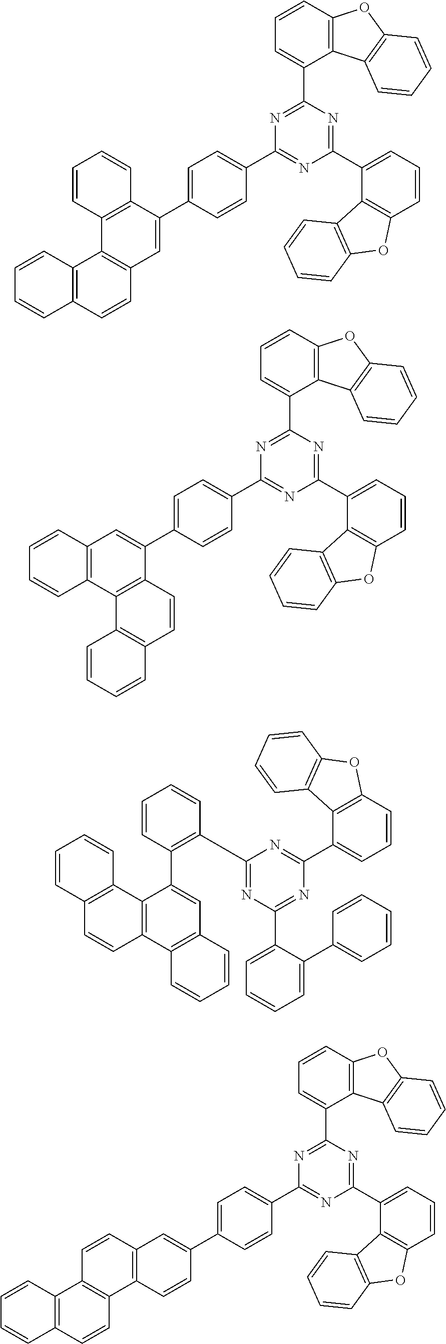 [SMILES: c1ccc2c(c1)ccc1cc(-c3ccc(-c4nc(-c5cccc6oc7ccccc7c56)nc(-c5cccc6oc7ccccc7c56)n4)cc3)c3ccccc3c12 ; c1ccc2c(c1)ccc1c(-c3ccc(-c4nc(-c5cccc6oc7ccccc7c56)nc(-c5cccc6oc7ccccc7c56)n4)cc3)cc3ccccc3c12 ; c1ccc(-c2ccccc2-c2nc(-c3ccccc3-c3cc4ccccc4c4ccc5ccccc5c34)nc(-c3cccc4oc5ccccc5c34)n2)cc1 ; c1ccc2c(c1)ccc1c3ccc(-c4ccc(-c5nc(-c6cccc7oc8ccccc8c67)nc(-c6cccc7oc8ccccc8c67)n5)cc4)cc3ccc21]

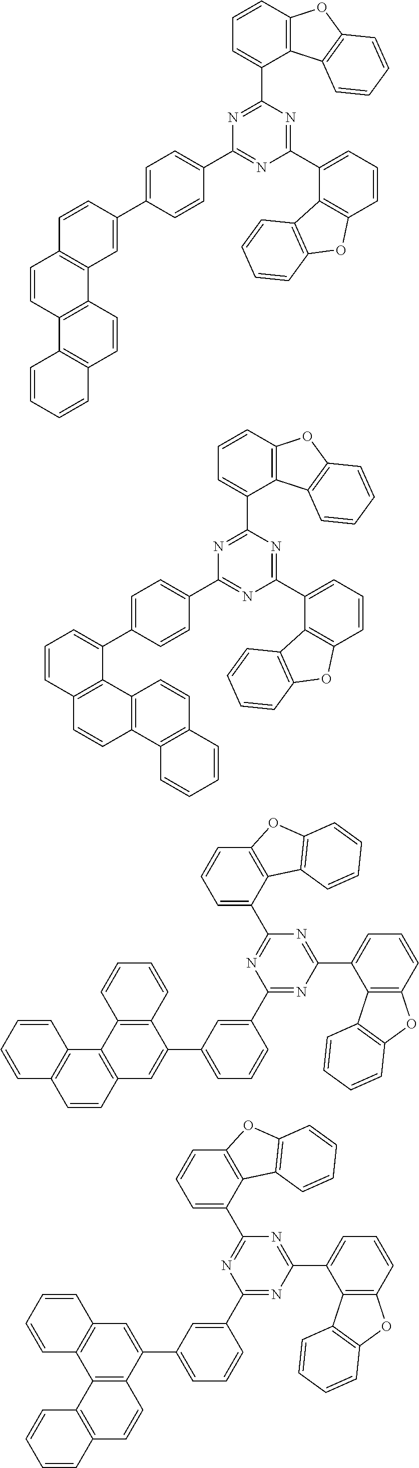 [SMILES: c1ccc2c(c1)ccc1c3cc(-c4ccc(-c5nc(-c6cccc7oc8ccccc8c67)nc(-c6cccc7oc8ccccc8c67)n5)cc4)ccc3ccc21 ; c1ccc2c(c1)ccc1c2ccc2cccc(-c3ccc(-c4nc(-c5cccc6oc7ccccc7c56)nc(-c5cccc6oc7ccccc7c56)n4)cc3)c21 ; c1cc(-c2nc(-c3cccc4oc5ccccc5c34)nc(-c3cccc4oc5ccccc5c34)n2)cc(-c2cc3ccc4ccccc4c3c3ccccc23)c1 ; c1cc(-c2nc(-c3cccc4oc5ccccc5c34)nc(-c3cccc4oc5ccccc5c34)n2)cc(-c2cc3ccccc3c3c2ccc2ccccc23)c1]

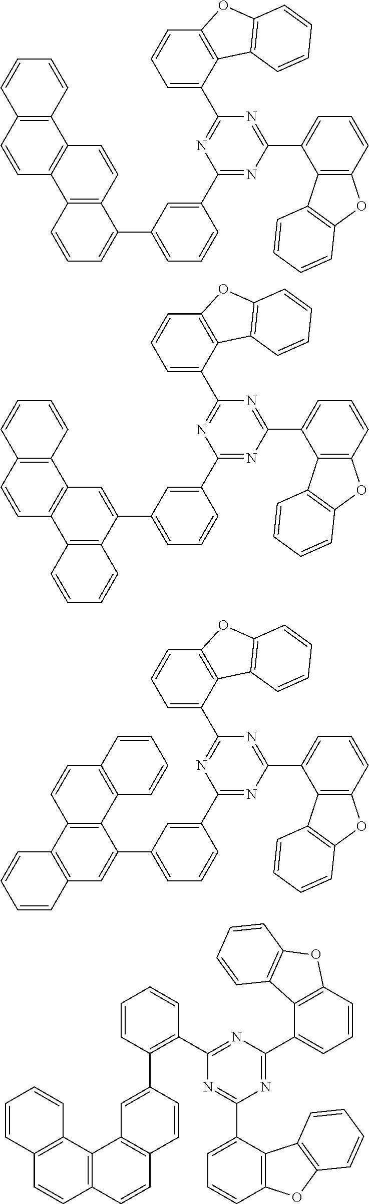

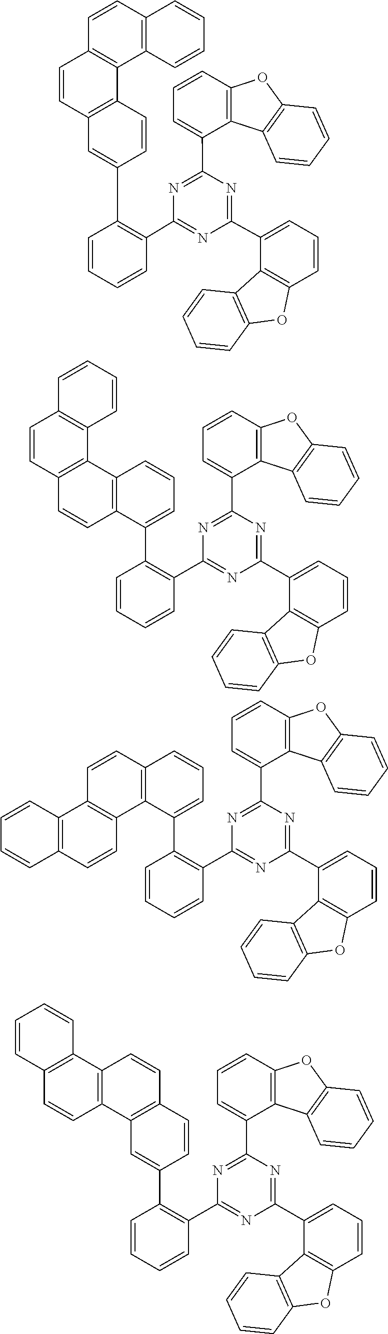

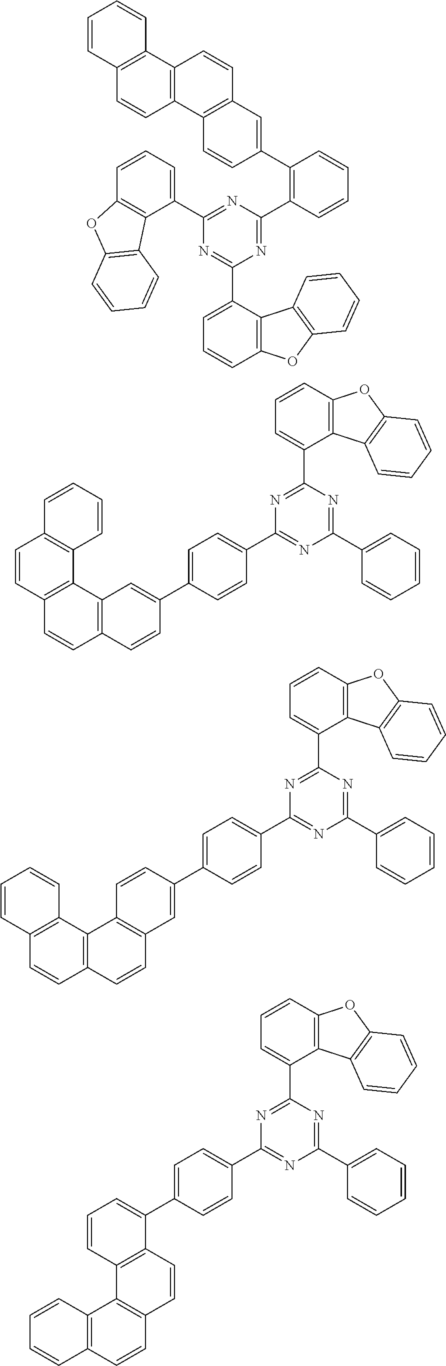

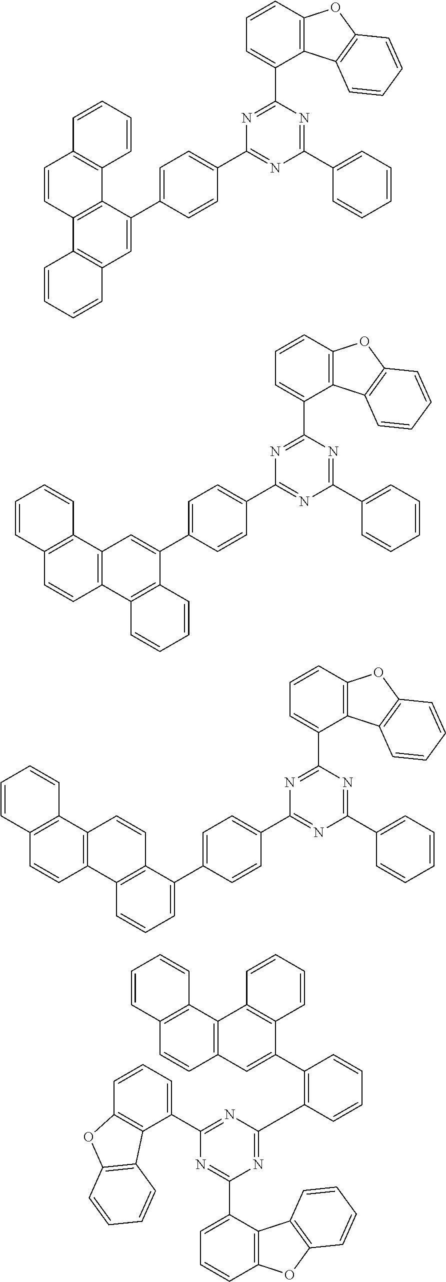

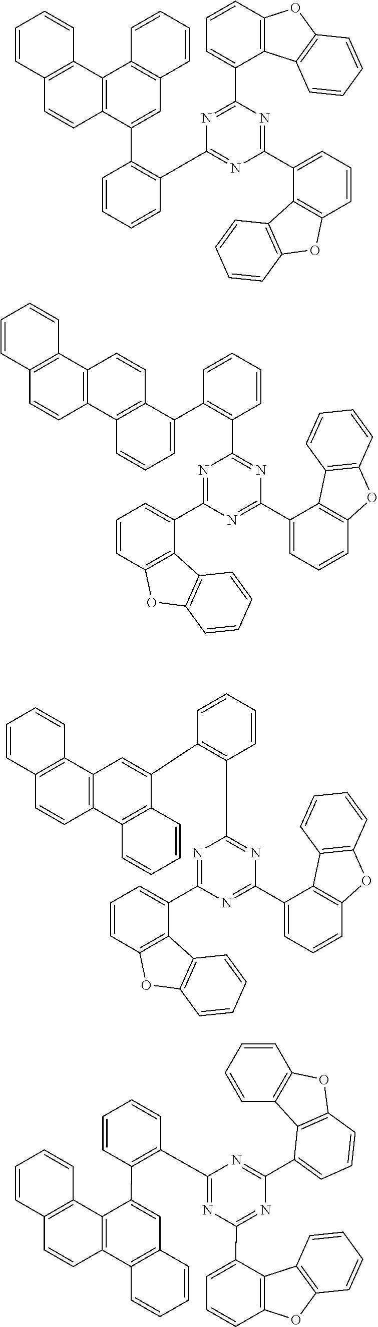

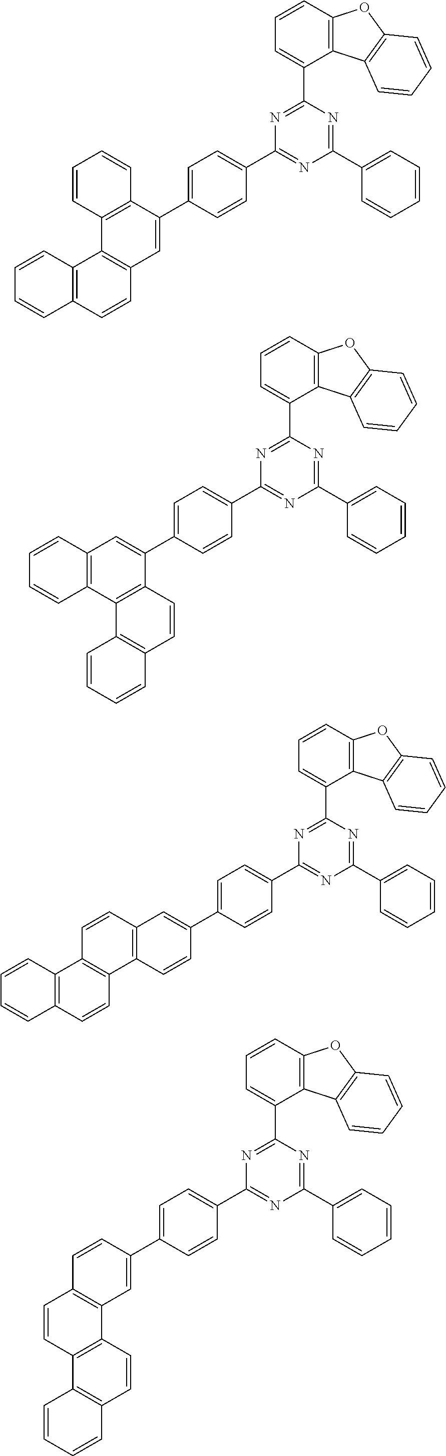 [SMILES: c1ccc(-c2nc(-c3ccc(-c4cc5ccc6ccccc6c5c5ccccc45)cc3)nc(-c3cccc4oc5ccccc5c34)n2)cc1 ; c1ccc(-c2nc(-c3ccc(-c4cc5ccccc5c5c4ccc4ccccc45)cc3)nc(-c3cccc4oc5ccccc5c34)n2)cc1 ; c1ccc(-c2nc(-c3ccc(-c4ccc5c(ccc6c7ccccc7ccc56)c4)cc3)nc(-c3cccc4oc5ccccc5c34)n2)cc1 ; c1ccc(-c2nc(-c3ccc(-c4ccc5ccc6c7ccccc7ccc6c5c4)cc3)nc(-c3cccc4oc5ccccc5c34)n2)cc1]

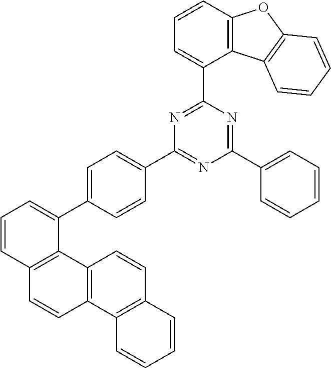 [SMILES: c1ccc(-c2nc(-c3ccc(-c4cccc5ccc6c7ccccc7ccc6c45)cc3)nc(-c3cccc4oc5ccccc5c34)n2)cc1]

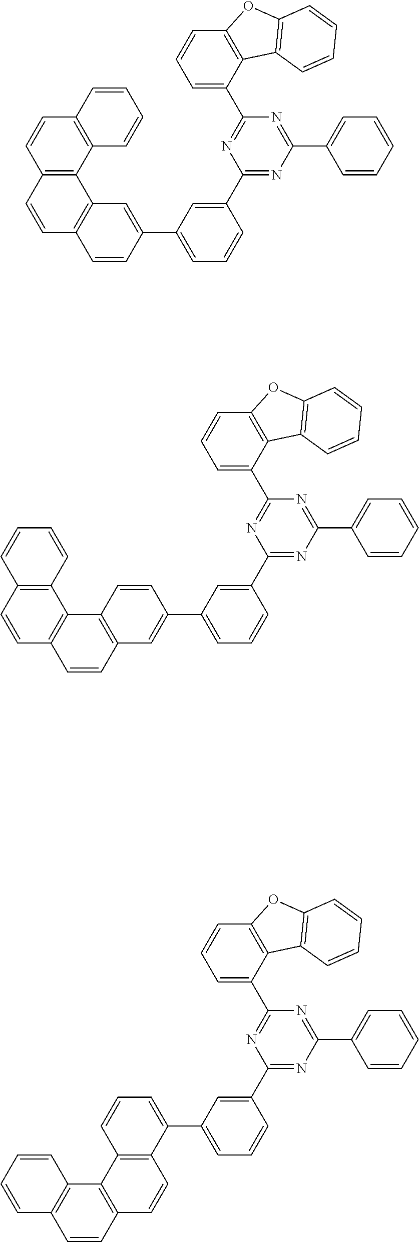 [SMILES: c1ccc(-c2nc(-c3cccc(-c4ccc5ccc6ccc7ccccc7c6c5c4)c3)nc(-c3cccc4oc5ccccc5c34)n2)cc1 ; c1ccc(-c2nc(-c3cccc(-c4ccc5c(ccc6ccc7ccccc7c65)c4)c3)nc(-c3cccc4oc5ccccc5c34)n2)cc1 ; c1ccc(-c2nc(-c3cccc(-c4cccc5c4ccc4ccc6ccccc6c45)c3)nc(-c3cccc4oc5ccccc5c34)n2)cc1]

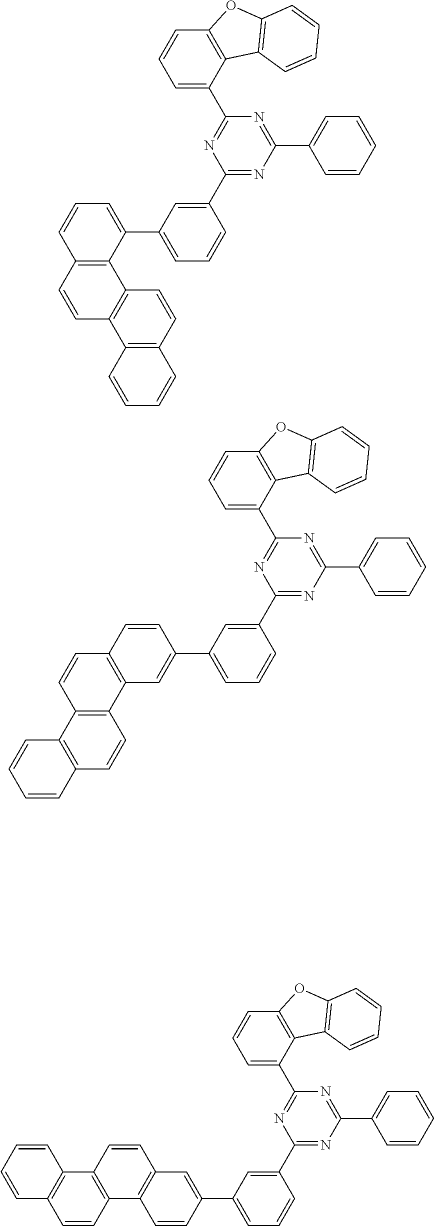 [SMILES: c1ccc(-c2nc(-c3cccc(-c4cccc5ccc6c7ccccc7ccc6c45)c3)nc(-c3cccc4oc5ccccc5c34)n2)cc1 ; c1ccc(-c2nc(-c3cccc(-c4ccc5ccc6c7ccccc7ccc6c5c4)c3)nc(-c3cccc4oc5ccccc5c34)n2)cc1 ; c1ccc(-c2nc(-c3cccc(-c4ccc5c(ccc6c7ccccc7ccc56)c4)c3)nc(-c3cccc4oc5ccccc5c34)n2)cc1]

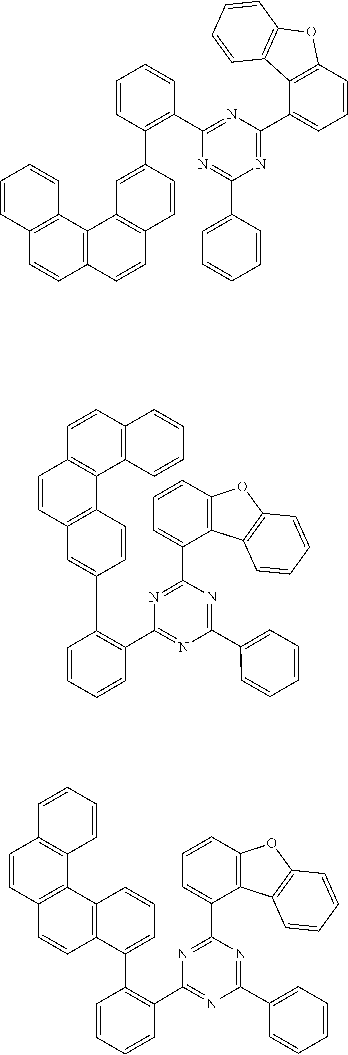 [SMILES: c1ccc(-c2nc(-c3ccccc3-c3ccc4ccc5ccc6ccccc6c5c4c3)nc(-c3cccc4oc5ccccc5c34)n2)cc1 ; c1ccc(-c2nc(-c3ccccc3-c3ccc4c(ccc5ccc6ccccc6c54)c3)nc(-c3cccc4oc5ccccc5c34)n2)cc1 ; c1ccc(-c2nc(-c3ccccc3-c3cccc4c3ccc3ccc5ccccc5c34)nc(-c3cccc4oc5ccccc5c34)n2)cc1]

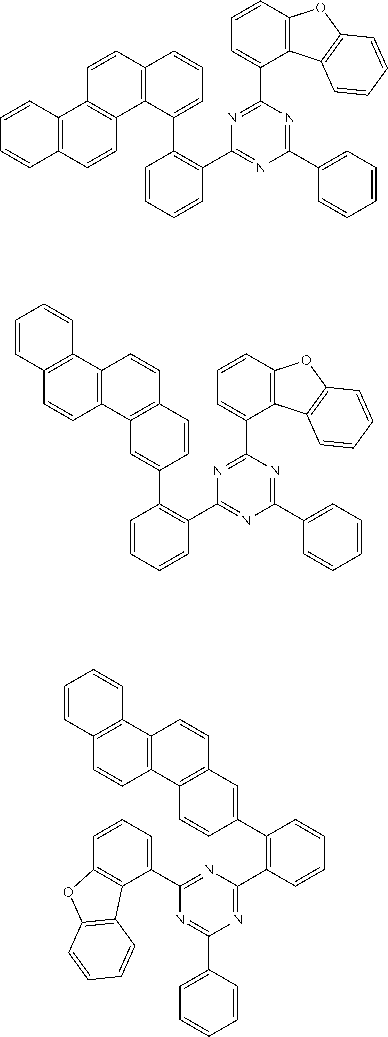 [SMILES: c1ccc(-c2nc(-c3ccccc3-c3cccc4ccc5c6ccccc6ccc5c34)nc(-c3cccc4oc5ccccc5c34)n2)cc1 ; c1ccc(-c2nc(-c3ccccc3-c3ccc4ccc5c6ccccc6ccc5c4c3)nc(-c3cccc4oc5ccccc5c34)n2)cc1 ; c1ccc(-c2nc(-c3ccccc3-c3ccc4c(ccc5c6ccccc6ccc45)c3)nc(-c3cccc4oc5ccccc5c34)n2)cc1]

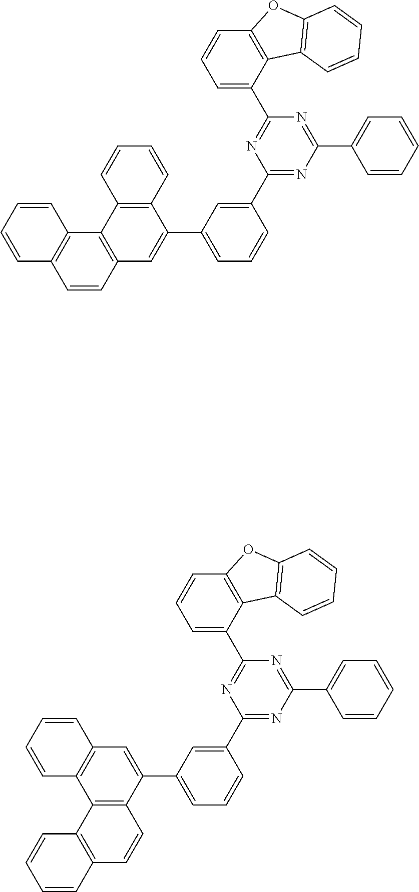 [SMILES: c1ccc(-c2nc(-c3cccc(-c4cc5ccc6ccccc6c5c5ccccc45)c3)nc(-c3cccc4oc5ccccc5c34)n2)cc1 ; c1ccc(-c2nc(-c3cccc(-c4cc5ccccc5c5c4ccc4ccccc45)c3)nc(-c3cccc4oc5ccccc5c34)n2)cc1]

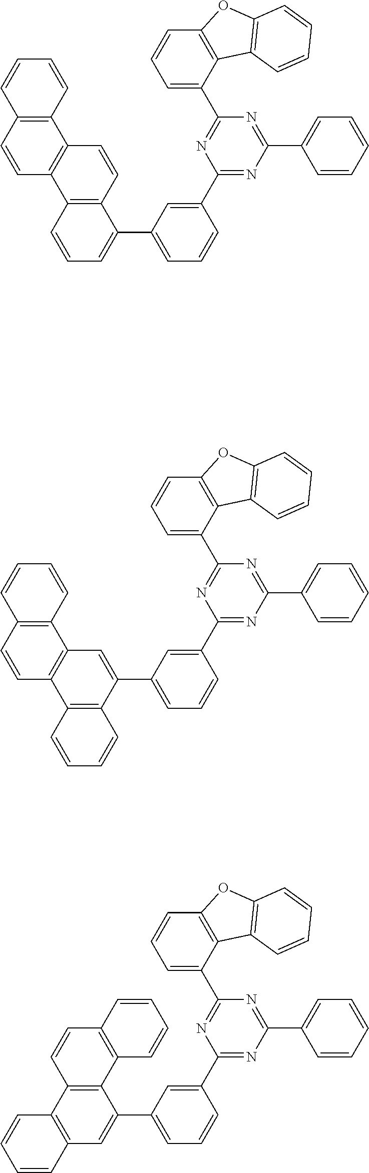 [SMILES: c1ccc(-c2nc(-c3cccc(-c4cccc5c4ccc4c6ccccc6ccc54)c3)nc(-c3cccc4oc5ccccc5c34)n2)cc1 ; c1ccc(-c2nc(-c3cccc(-c4cc5c6ccccc6ccc5c5ccccc45)c3)nc(-c3cccc4oc5ccccc5c34)n2)cc1 ; c1ccc(-c2nc(-c3cccc(-c4cc5ccccc5c5ccc6ccccc6c45)c3)nc(-c3cccc4oc5ccccc5c34)n2)cc1]

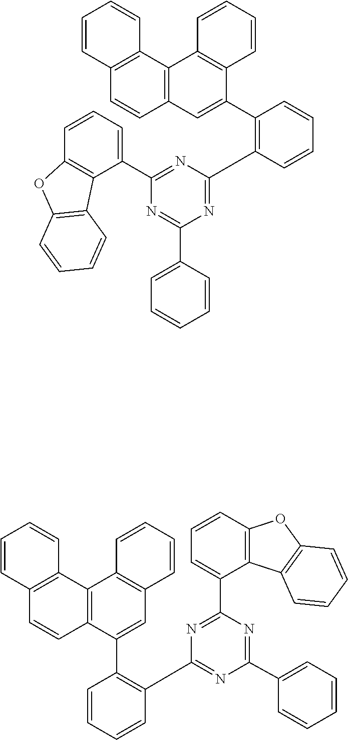 [SMILES: c1ccc(-c2nc(-c3ccccc3-c3cc4ccc5ccccc5c4c4ccccc34)nc(-c3cccc4oc5ccccc5c34)n2)cc1 ; c1ccc(-c2nc(-c3ccccc3-c3cc4ccccc4c4c3ccc3ccccc34)nc(-c3cccc4oc5ccccc5c34)n2)cc1]

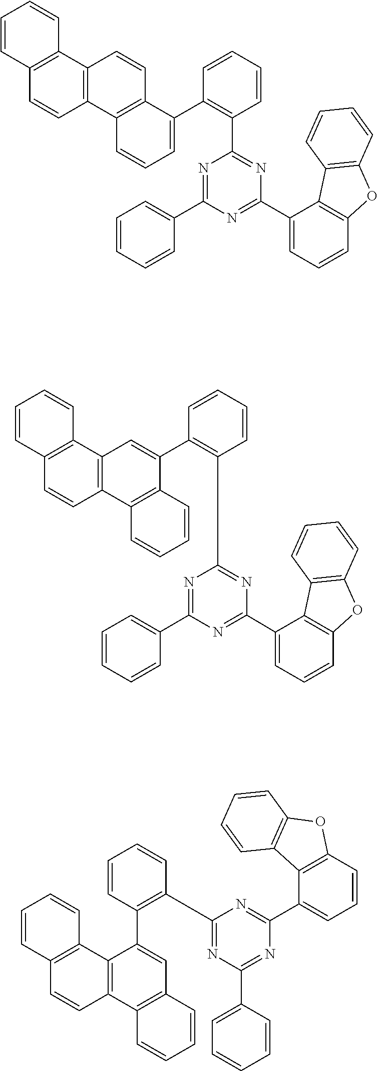 [SMILES: c1ccc(-c2nc(-c3ccccc3-c3cccc4c3ccc3c5ccccc5ccc43)nc(-c3cccc4oc5ccccc5c34)n2)cc1 ; c1ccc(-c2nc(-c3ccccc3-c3cc4c5ccccc5ccc4c4ccccc34)nc(-c3cccc4oc5ccccc5c34)n2)cc1 ; c1ccc(-c2nc(-c3ccccc3-c3cc4ccccc4c4ccc5ccccc5c34)nc(-c3cccc4oc5ccccc5c34)n2)cc1]

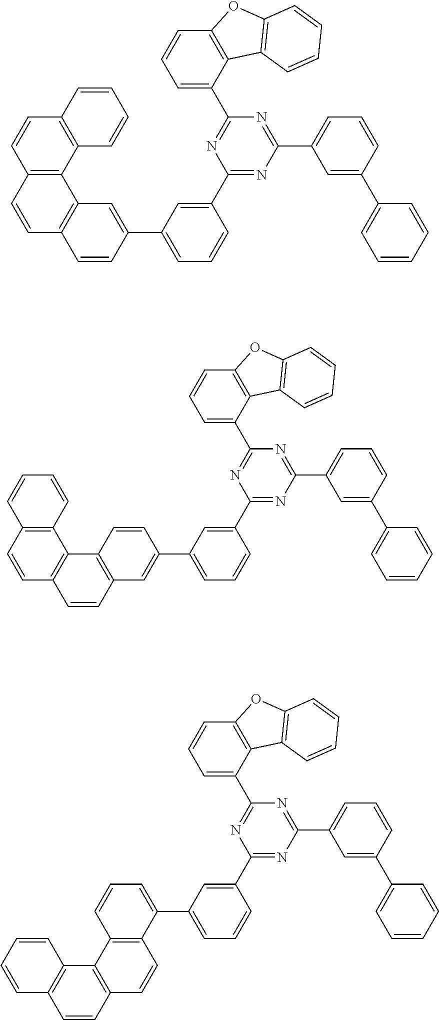 [SMILES: c1ccc(-c2cccc(-c3nc(-c4cccc(-c5ccc6ccc7ccc8ccccc8c7c6c5)c4)nc(-c4cccc5oc6ccccc6c45)n3)c2)cc1 ; c1ccc(-c2cccc(-c3nc(-c4cccc(-c5ccc6c(ccc7ccc8ccccc8c76)c5)c4)nc(-c4cccc5oc6ccccc6c45)n3)c2)cc1 ; c1ccc(-c2cccc(-c3nc(-c4cccc(-c5cccc6c5ccc5ccc7ccccc7c56)c4)nc(-c4cccc5oc6ccccc6c45)n3)c2)cc1]

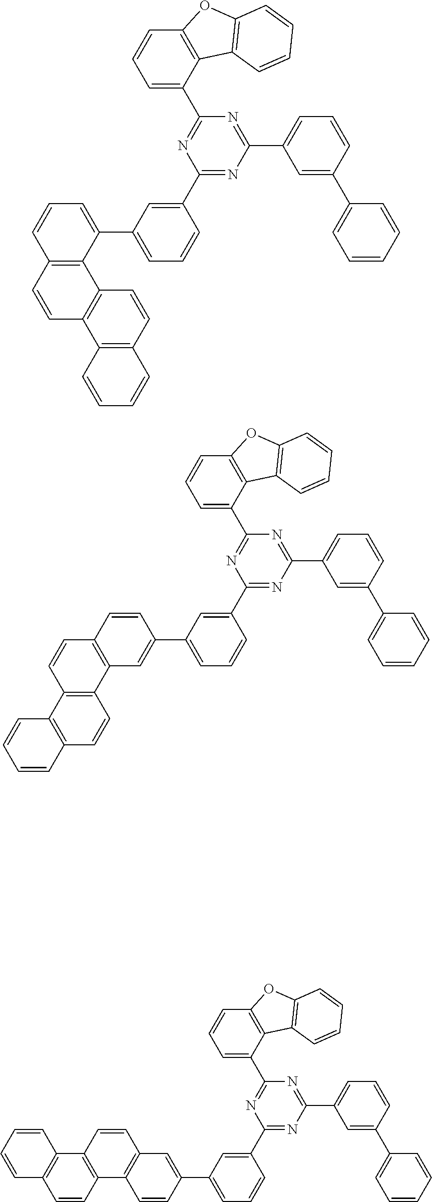 [SMILES: c1ccc(-c2cccc(-c3nc(-c4cccc(-c5cccc6ccc7c8ccccc8ccc7c56)c4)nc(-c4cccc5oc6ccccc6c45)n3)c2)cc1 ; c1ccc(-c2cccc(-c3nc(-c4cccc(-c5ccc6ccc7c8ccccc8ccc7c6c5)c4)nc(-c4cccc5oc6ccccc6c45)n3)c2)cc1 ; c1ccc(-c2cccc(-c3nc(-c4cccc(-c5ccc6c(ccc7c8ccccc8ccc67)c5)c4)nc(-c4cccc5oc6ccccc6c45)n3)c2)cc1]

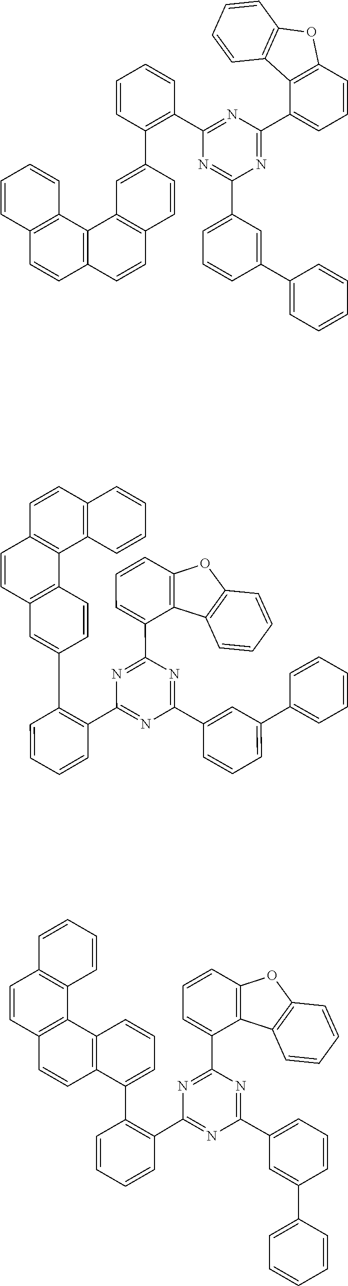 [SMILES: c1ccc(-c2cccc(-c3nc(-c4ccccc4-c4ccc5ccc6ccc7ccccc7c6c5c4)nc(-c4cccc5oc6ccccc6c45)n3)c2)cc1 ; c1ccc(-c2cccc(-c3nc(-c4ccccc4-c4ccc5c(ccc6ccc7ccccc7c65)c4)nc(-c4cccc5oc6ccccc6c45)n3)c2)cc1 ; c1ccc(-c2cccc(-c3nc(-c4ccccc4-c4cccc5c4ccc4ccc6ccccc6c45)nc(-c4cccc5oc6ccccc6c45)n3)c2)cc1]

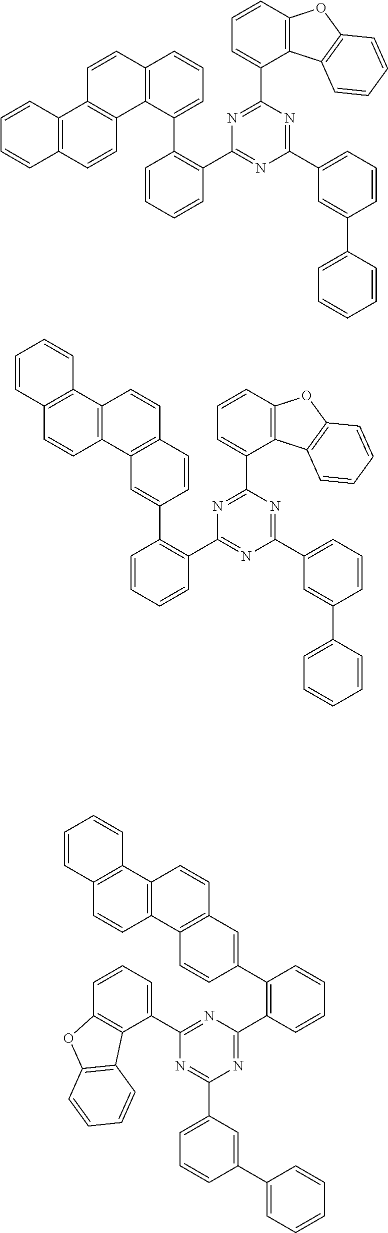 [SMILES: c1ccc(-c2cccc(-c3nc(-c4ccccc4-c4cccc5ccc6c7ccccc7ccc6c45)nc(-c4cccc5oc6ccccc6c45)n3)c2)cc1 ; c1ccc(-c2cccc(-c3nc(-c4ccccc4-c4ccc5ccc6c7ccccc7ccc6c5c4)nc(-c4cccc5oc6ccccc6c45)n3)c2)cc1 ; c1ccc(-c2cccc(-c3nc(-c4ccccc4-c4ccc5c(ccc6c7ccccc7ccc56)c4)nc(-c4cccc5oc6ccccc6c45)n3)c2)cc1]

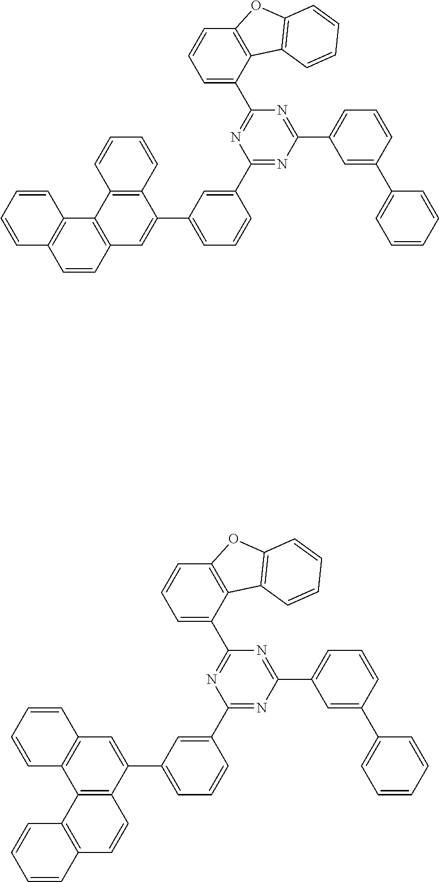 [SMILES: c1ccc(-c2cccc(-c3nc(-c4cccc(-c5cc6ccc7ccccc7c6c6ccccc56)c4)nc(-c4cccc5oc6ccccc6c45)n3)c2)cc1 ; c1ccc(-c2cccc(-c3nc(-c4cccc(-c5cc6ccccc6c6c5ccc5ccccc56)c4)nc(-c4cccc5oc6ccccc6c45)n3)c2)cc1]

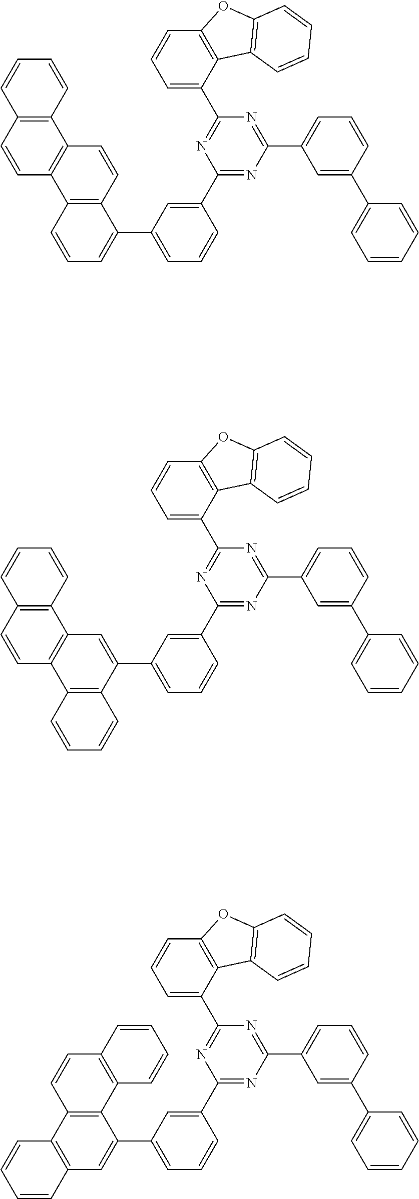 [SMILES: c1ccc(-c2cccc(-c3nc(-c4cccc(-c5cccc6c5ccc5c7ccccc7ccc65)c4)nc(-c4cccc5oc6ccccc6c45)n3)c2)cc1 ; c1ccc(-c2cccc(-c3nc(-c4cccc(-c5cc6c7ccccc7ccc6c6ccccc56)c4)nc(-c4cccc5oc6ccccc6c45)n3)c2)cc1 ; c1ccc(-c2cccc(-c3nc(-c4cccc(-c5cc6ccccc6c6ccc7ccccc7c56)c4)nc(-c4cccc5oc6ccccc6c45)n3)c2)cc1]

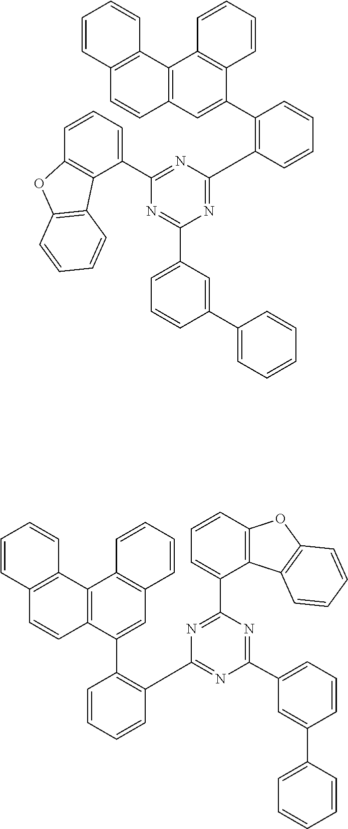 [SMILES: c1ccc(-c2cccc(-c3nc(-c4ccccc4-c4cc5ccc6ccccc6c5c5ccccc45)nc(-c4cccc5oc6ccccc6c45)n3)c2)cc1 ; c1ccc(-c2cccc(-c3nc(-c4ccccc4-c4cc5ccccc5c5c4ccc4ccccc45)nc(-c4cccc5oc6ccccc6c45)n3)c2)cc1]

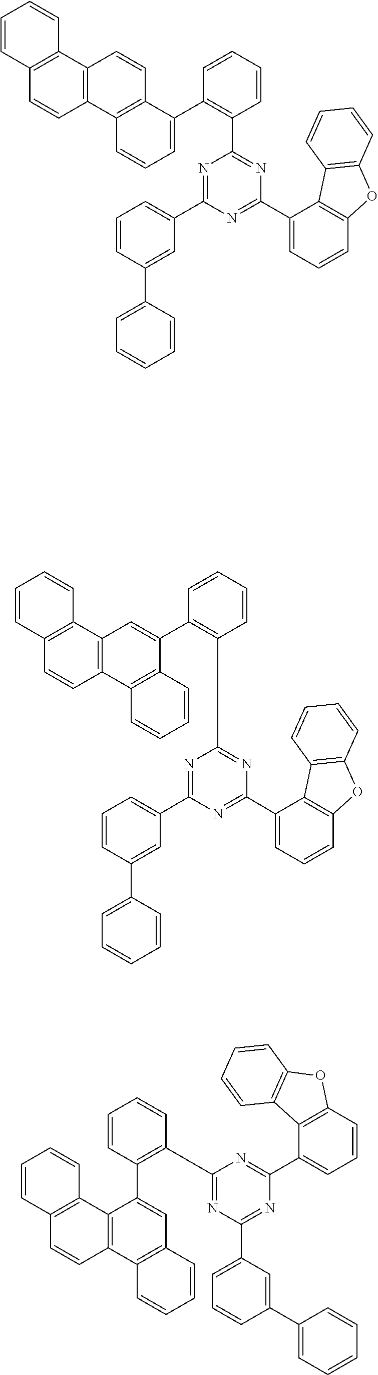 [SMILES: c1ccc(-c2cccc(-c3nc(-c4ccccc4-c4cccc5c4ccc4c6ccccc6ccc54)nc(-c4cccc5oc6ccccc6c45)n3)c2)cc1 ; c1ccc(-c2cccc(-c3nc(-c4ccccc4-c4cc5c6ccccc6ccc5c5ccccc45)nc(-c4cccc5oc6ccccc6c45)n3)c2)cc1 ; c1ccc(-c2cccc(-c3nc(-c4ccccc4-c4cc5ccccc5c5ccc6ccccc6c45)nc(-c4cccc5oc6ccccc6c45)n3)c2)cc1]

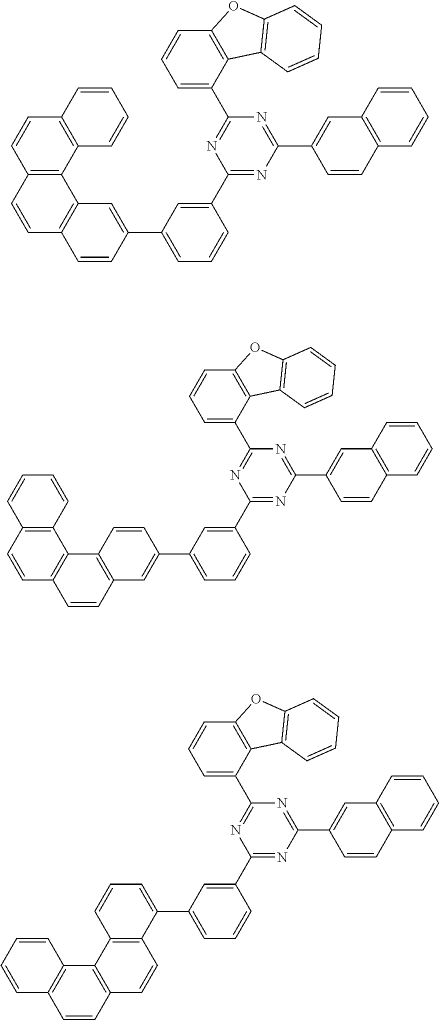 [SMILES: c1cc(-c2ccc3ccc4ccc5ccccc5c4c3c2)cc(-c2nc(-c3ccc4ccccc4c3)nc(-c3cccc4oc5ccccc5c34)n2)c1 ; c1cc(-c2ccc3c(ccc4ccc5ccccc5c43)c2)cc(-c2nc(-c3ccc4ccccc4c3)nc(-c3cccc4oc5ccccc5c34)n2)c1 ; c1cc(-c2nc(-c3ccc4ccccc4c3)nc(-c3cccc4oc5ccccc5c34)n2)cc(-c2cccc3c2ccc2ccc4ccccc4c23)c1]

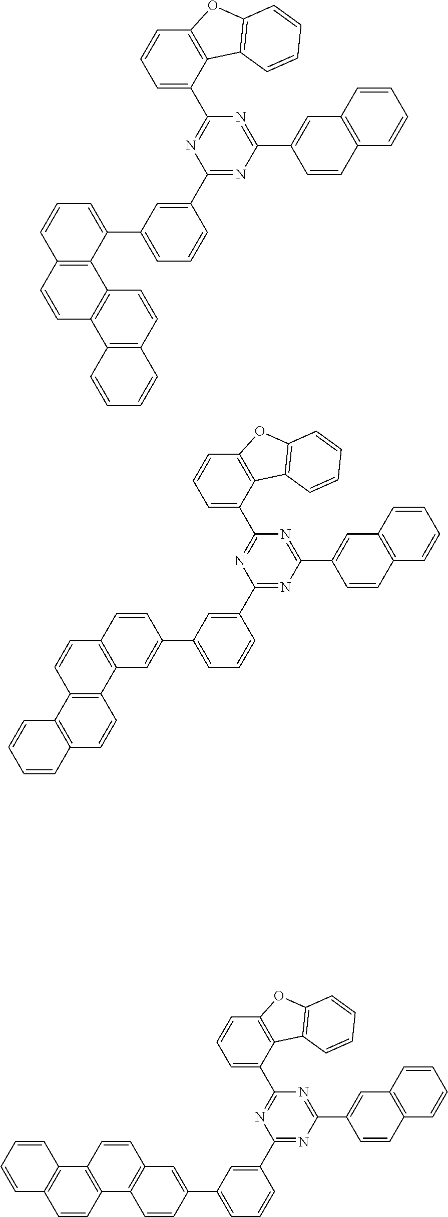 [SMILES: c1cc(-c2nc(-c3ccc4ccccc4c3)nc(-c3cccc4oc5ccccc5c34)n2)cc(-c2cccc3ccc4c5ccccc5ccc4c23)c1 ; c1cc(-c2ccc3ccc4c5ccccc5ccc4c3c2)cc(-c2nc(-c3ccc4ccccc4c3)nc(-c3cccc4oc5ccccc5c34)n2)c1 ; c1cc(-c2ccc3c(ccc4c5ccccc5ccc34)c2)cc(-c2nc(-c3ccc4ccccc4c3)nc(-c3cccc4oc5ccccc5c34)n2)c1]

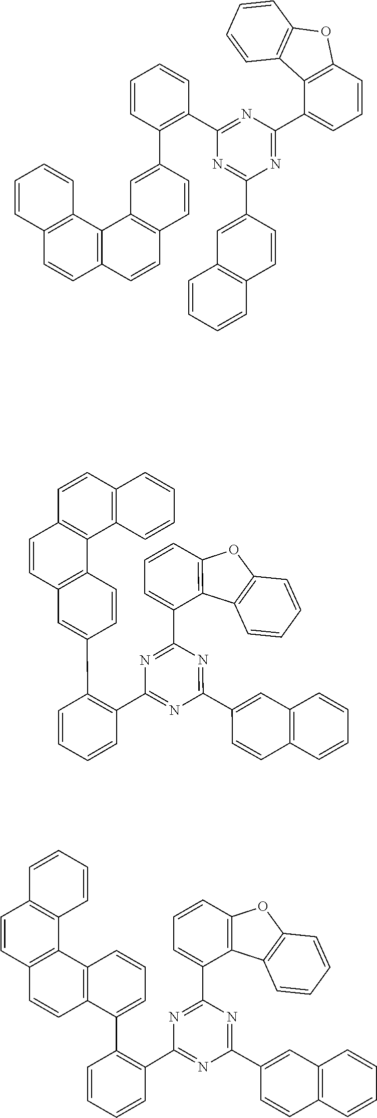 [SMILES: c1ccc(-c2nc(-c3ccc4ccccc4c3)nc(-c3cccc4oc5ccccc5c34)n2)c(-c2ccc3ccc4ccc5ccccc5c4c3c2)c1 ; c1ccc(-c2nc(-c3ccc4ccccc4c3)nc(-c3cccc4oc5ccccc5c34)n2)c(-c2ccc3c(ccc4ccc5ccccc5c43)c2)c1 ; c1ccc(-c2cccc3c2ccc2ccc4ccccc4c23)c(-c2nc(-c3ccc4ccccc4c3)nc(-c3cccc4oc5ccccc5c34)n2)c1]

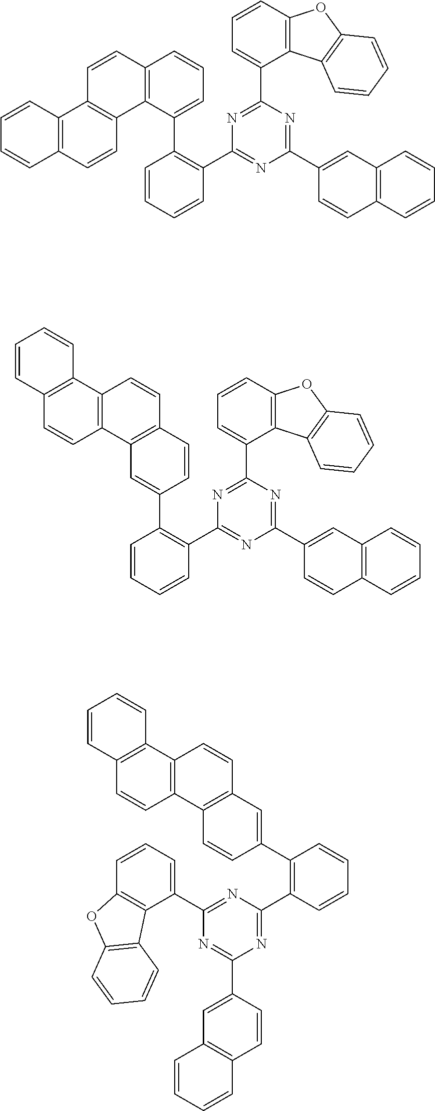 [SMILES: c1ccc(-c2cccc3ccc4c5ccccc5ccc4c23)c(-c2nc(-c3ccc4ccccc4c3)nc(-c3cccc4oc5ccccc5c34)n2)c1 ; c1ccc(-c2nc(-c3ccc4ccccc4c3)nc(-c3cccc4oc5ccccc5c34)n2)c(-c2ccc3ccc4c5ccccc5ccc4c3c2)c1 ; c1ccc(-c2nc(-c3ccc4ccccc4c3)nc(-c3cccc4oc5ccccc5c34)n2)c(-c2ccc3c(ccc4c5ccccc5ccc34)c2)c1]

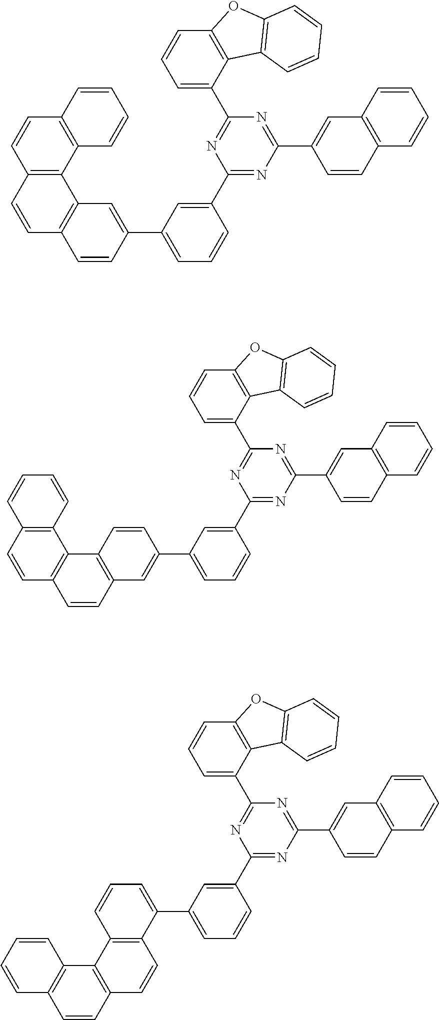 [SMILES: c1cc(-c2ccc3ccc4ccc5ccccc5c4c3c2)cc(-c2nc(-c3ccc4ccccc4c3)nc(-c3cccc4oc5ccccc5c34)n2)c1 ; c1cc(-c2ccc3c(ccc4ccc5ccccc5c43)c2)cc(-c2nc(-c3ccc4ccccc4c3)nc(-c3cccc4oc5ccccc5c34)n2)c1 ; c1cc(-c2nc(-c3ccc4ccccc4c3)nc(-c3cccc4oc5ccccc5c34)n2)cc(-c2cccc3c2ccc2ccc4ccccc4c23)c1]

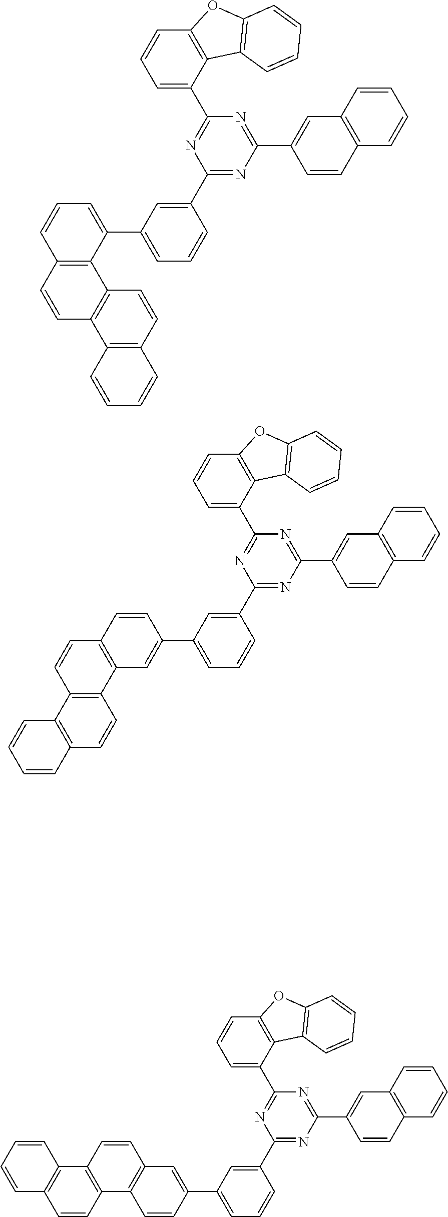 [SMILES: c1cc(-c2nc(-c3ccc4ccccc4c3)nc(-c3cccc4oc5ccccc5c34)n2)cc(-c2cccc3ccc4c5ccccc5ccc4c23)c1 ; c1cc(-c2ccc3ccc4c5ccccc5ccc4c3c2)cc(-c2nc(-c3ccc4ccccc4c3)nc(-c3cccc4oc5ccccc5c34)n2)c1 ; c1cc(-c2ccc3c(ccc4c5ccccc5ccc34)c2)cc(-c2nc(-c3ccc4ccccc4c3)nc(-c3cccc4oc5ccccc5c34)n2)c1]

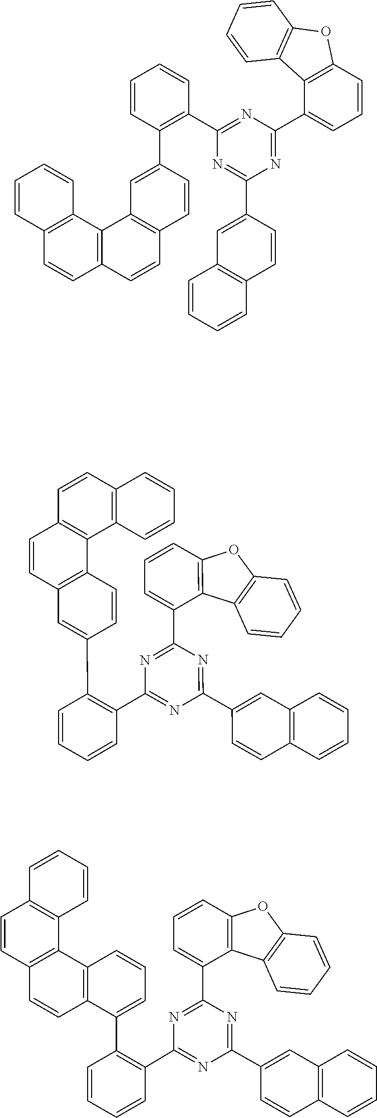 [SMILES: c1ccc(-c2nc(-c3ccc4ccccc4c3)nc(-c3cccc4oc5ccccc5c34)n2)c(-c2ccc3ccc4ccc5ccccc5c4c3c2)c1 ; c1ccc(-c2nc(-c3ccc4ccccc4c3)nc(-c3cccc4oc5ccccc5c34)n2)c(-c2ccc3c(ccc4ccc5ccccc5c43)c2)c1 ; c1ccc(-c2cccc3c2ccc2ccc4ccccc4c23)c(-c2nc(-c3ccc4ccccc4c3)nc(-c3cccc4oc5ccccc5c34)n2)c1]

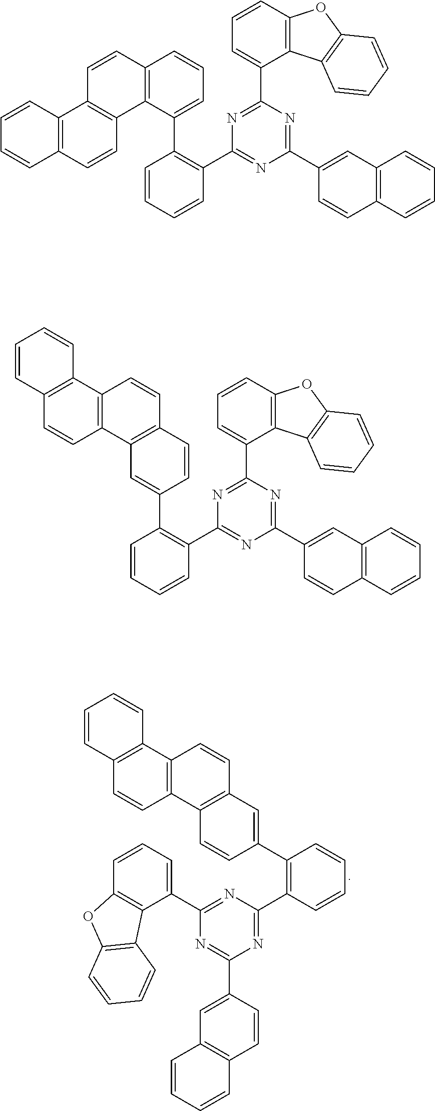 [SMILES: c1ccc(-c2cccc3ccc4c5ccccc5ccc4c23)c(-c2nc(-c3ccc4ccccc4c3)nc(-c3cccc4oc5ccccc5c34)n2)c1 ; c1ccc(-c2nc(-c3ccc4ccccc4c3)nc(-c3cccc4oc5ccccc5c34)n2)c(-c2ccc3ccc4c5ccccc5ccc4c3c2)c1 ; c1ccc(-c2nc(-c3ccc4ccccc4c3)nc(-c3cccc4oc5ccccc5c34)n2)c(-c2ccc3c(ccc4c5ccccc5ccc34)c2)c1]

Meanwhile, the compound of Chemical Formula 1 can be prepared by, for example, a preparation method as shown in one of Reaction Schemes 1-1 to 1-5 below, and other compounds can be prepared similarly.

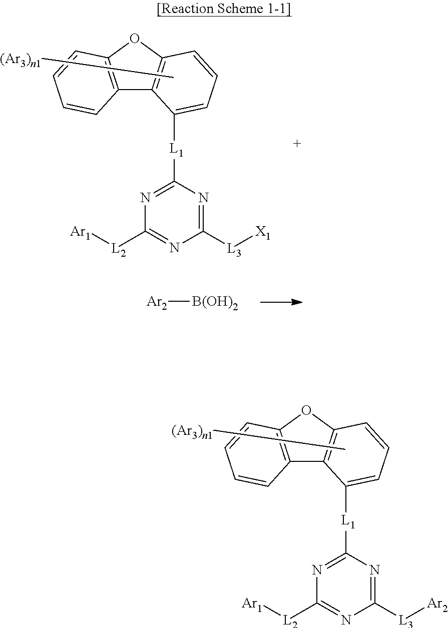

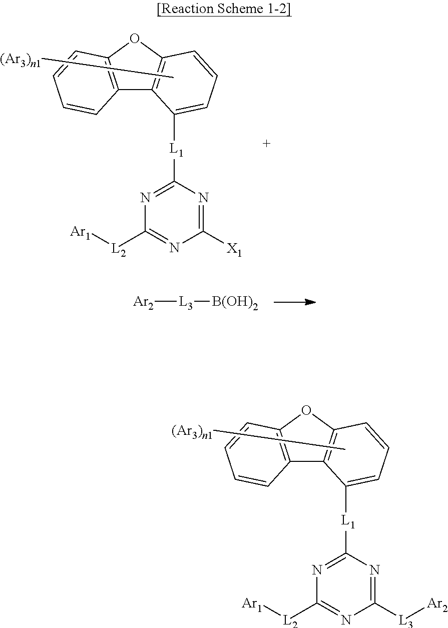

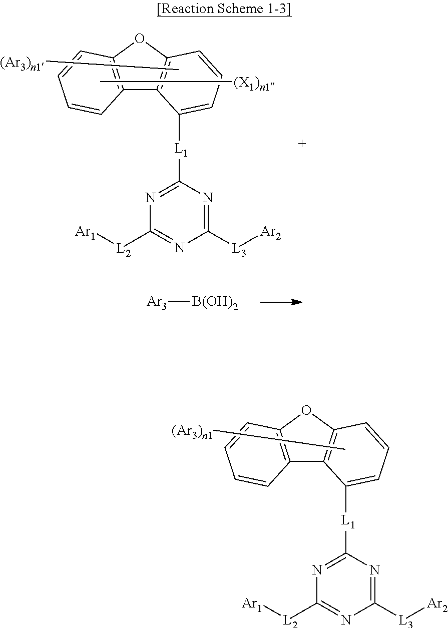

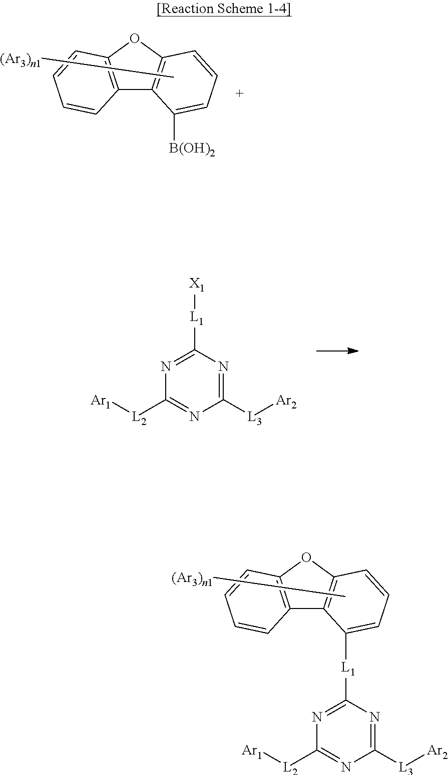

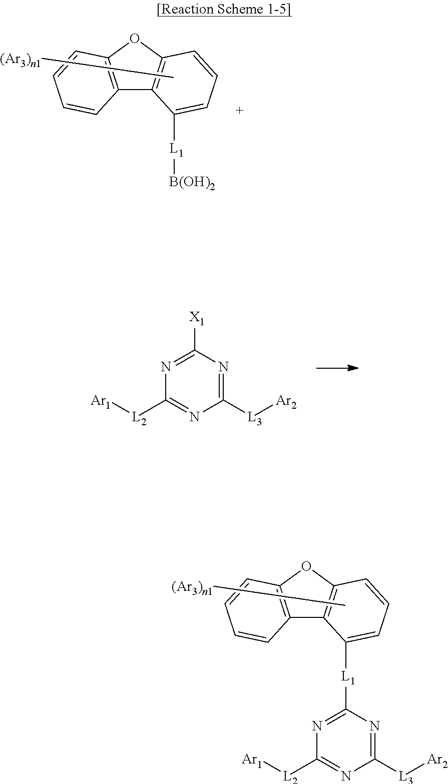

In Reaction Schemes 1-1 to 1-5, L₁ to L₃, Ar₁ to Ar₃, and n1 are each independently as defined in Chemical Formula 1, n1′ is an integer from 0 to 6, n1″ is an integer from 1 to 7, and each X₁ is independently a halogen. Preferably, each X₁ is independently chlorine or bromine. Also, the sum of n1′ and n1″ is an integer from 1 to 7.

Preferably, the compound of Chemical Formula 1 can be produced by Reaction Scheme 1-1 or 1-3.

The Reaction Schemes 1-1 to 1-5 are performed as Suzuki-coupling reactions. The Suzuki-coupling reaction is preferably performed in the presence of a palladium catalyst and a base, respectively, and the reactive group for the Suzuki-coupling reaction can be appropriately changed. The above preparation method can be further embodied in the Preparation Examples described hereinafter.

For example, in Reaction Schemes 1-1 to 1-5, sodium tert-butoxide (NaOtBu), potassium carbonate (K₂CO₃), potassium phosphate (K₃PO₄), sodium tert-butoxide (NaOtBu), sodium bicarbonate (NaHCO₃), cesium carbonate (Cs₂CO₃), sodium acetate (NaOAc), potassium acetate (KOAc), sodium ethoxide (NaOEt), triethylamine (Et₃N), N,N-diisopropylethylamine (EtN(iPr)₂), or the like can be used as the base component. Preferably, the base component can be sodium tert-butoxide (NaOtBu), potassium carbonate (K₂CO₃), potassium phosphate (K₃PO₄), cesium carbonate (Cs₂CO₃), potassium acetate (KOAc), or N,N-diisopropylethylamine (EtN(iPr)₂). In particular, potassium carbonate (K₂CO₃), or potassium phosphate (K₃PO₄) can be used as the base component.

In addition, in Reaction Schemes 1-1 to 1-5, bis(tri-(tert-butyl)-phosphine)palladium (0) (Pd(P-tBu₃)₂), tetrakis(triphenylphosphine)-palladium (0) (Pd(PPh₃)₄), tris(dibenzylideneacetone)-dipalladium (0) (Pd₂(dba)₃), bis(dibenzylideneacetone)palladium (0) (Pd(dba)₂), palladium (II) acetate (Pd(OAc)₂), or the like can be used as the palladium catalyst. Preferably, the palladium catalyst can be bis(tri-(tert-butyl)phosphine)palladium (0) (Pd(P-tBu₃)₂), tetrakis(triphenylphosphine)-palladium (0) (Pd(PPh₃)₄), or bis(dibenzylideneacetone)palladium (0) (Pd(dba)₂). In particular, in the Reaction Scheme 1, tetrakis(triphenylphosphine)palladium (0) (Pd(PPh₃)₄), or bis(tri-(tert-butyl)phosphine)palladium (0) (Pd(P-tBu₃)₂) can be used as the palladium catalyst.

### (Second Compound)

The second compound is the following Chemical Formula 2. Specifically, it has a structure in which a tertiary amine group is bonded to the central benzene ring of the phenanthrene-based core through an arylene linker L₇. The second compound is characterized by the tertiary amine group being bonded to the core of the phenanthrene-based polycyclic ring. In particular, the second compound according to the present disclosure has excellent electron transport capability, compared to a compound having a different structure or substituents, that is, a compound in which the tertiary amine group is bonded to a benzene ring other than the central benzene ring of the phenanthrene-based core. Accordingly, the second compound according to the present disclosure has the specific structure and substituents as described above to increase the probability of recombination of holes and electrons in the light emitting layer together with the first compound by efficiently delivering electrons to a dopant material.

In Chemical Formula 2 related to the second compound included in the organic light-emitting device of the present disclosure,
Ar₄ is hydrogen, a substituted or unsubstituted C₆₋₆₀ aryl, or a substituted or unsubstituted C₂₋₆₀ heteroaryl containing at least one heteroatom selected from the group consisting of N, O and S,
Ar₅ and Ar₆ are each a substituted or unsubstituted C₆₋₆₀ aryl or a substituted or unsubstituted C₂₋₆₀ heteroaryl containing at least one heteroatom selected from the group consisting of N, O and S,
L₄ to L₆ are each independently a single bond, a substituted or unsubstituted C₆₋₆₀ arylene, or a substituted or unsubstituted C₂₋₆₀ heteroarylene containing at least one heteroatom selected from the group consisting of N, O and S,
L₇ is a substituted or unsubstituted C₆₋₆₀ arylene, and
all hydrogens included in in Chemical Formula 2 can each independently be replaced with deuterium.

Preferably, Ar₄ can be hydrogen, a substituted or unsubstituted C₆₋₂₀ aryl, or a substituted or unsubstituted C₂₋₂₀ heteroaryl containing at least one heteroatom selected from the group consisting of N, O and S.

More preferably, Ar₄ can be hydrogen, phenyl, naphthyl, or biphenyl.

Preferably, Ar₅ and Ar₆ can each be a substituted or unsubstituted C₆₋₂₀ aryl or a substituted or unsubstituted C₂₋₂₀ heteroaryl containing at least one heteroatom selected from the group consisting of N, O and S.

More preferably, Ar₅ and Ar₆ can each independently be phenyl, phenyl substituted with five deuteriums, phenyl substituted with naphthyl, biphenyl, biphenyl substituted with four deuteriums, biphenyl substituted with nine deuteriums, terphenyl, terphenyl substituted with four deuteriums, quaterphenyl, naphthyl, naphthyl substituted with phenyl, phenanthrenyl, triphenylene, dimethylfluorenyl, diphenylfluorenyl, carbazolyl, phenylcarbazolyl, dibenzofuranyl, dibenzothiophenyl, or dibenzofuranyl substituted with phenyl.

In addition, Ar₅ and Ar₆ can each independently be any one group selected from the group consisting of the following groups:

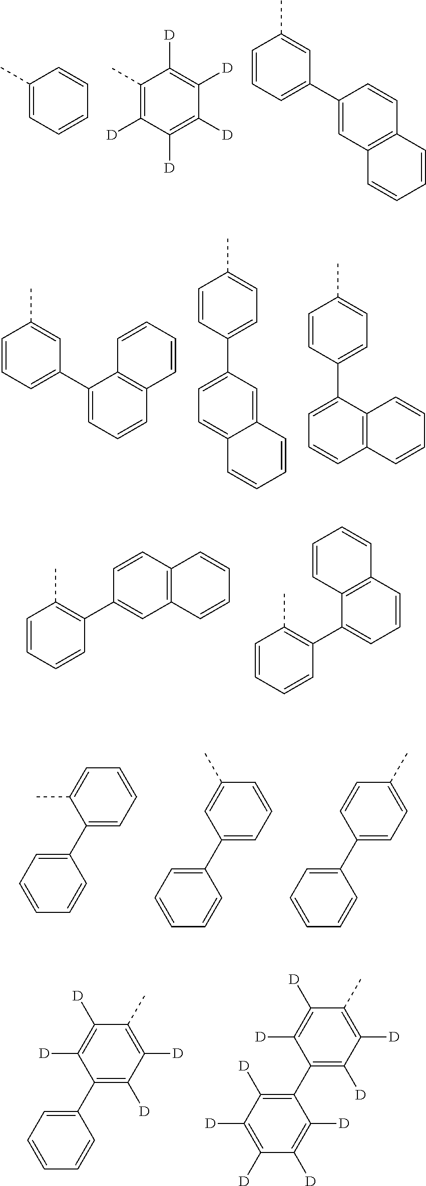

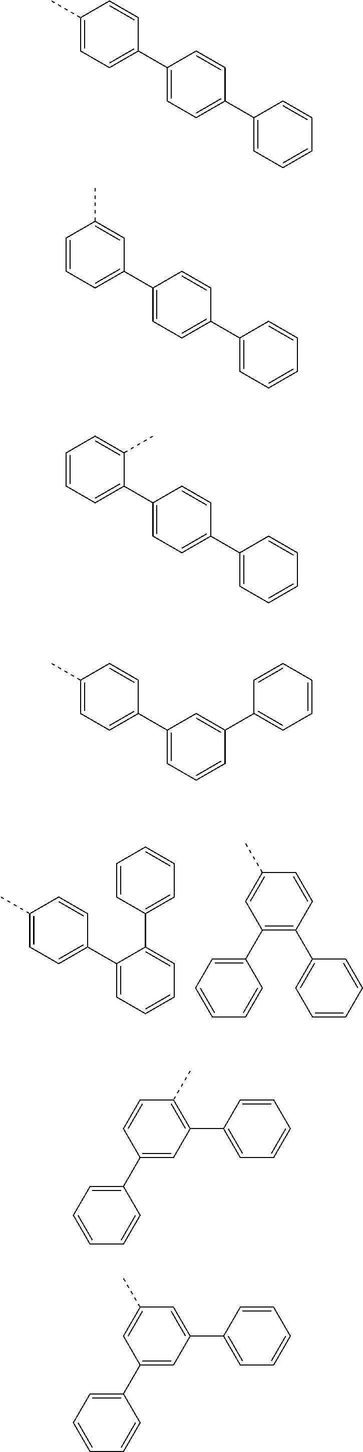

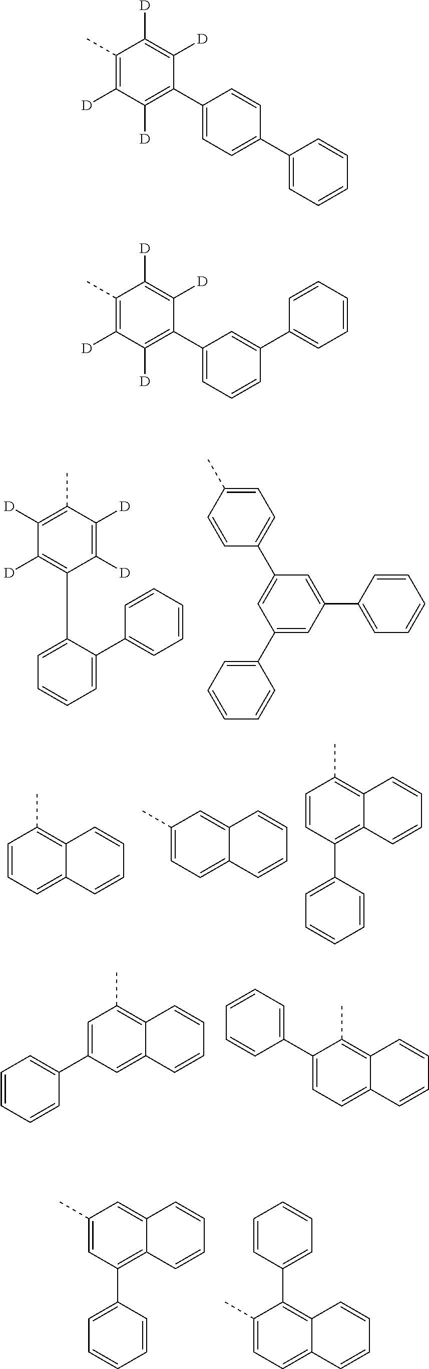

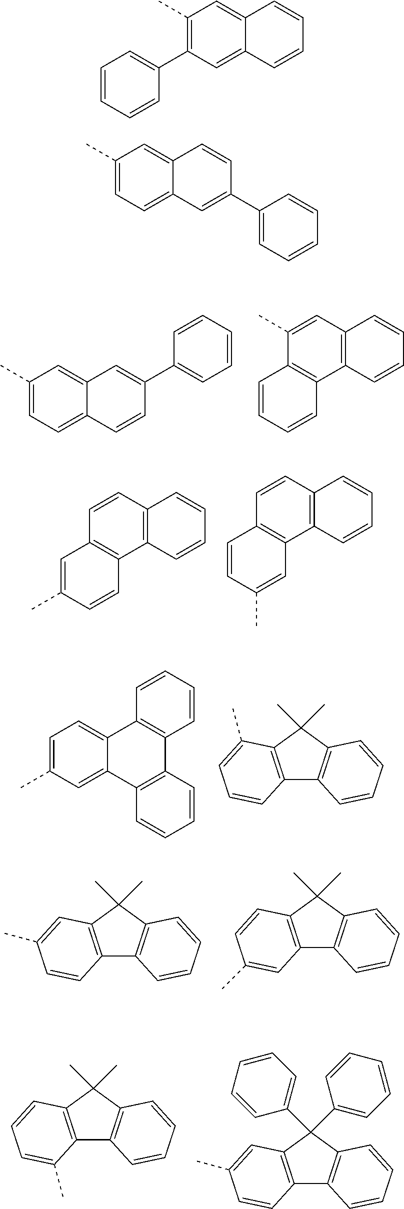

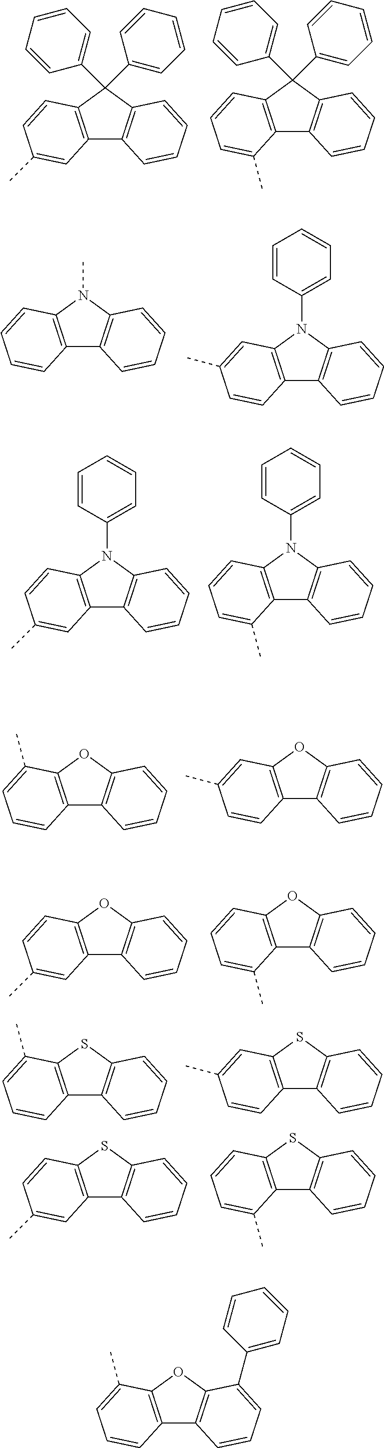

wherein in the above formulae, D represents deuterium.

Preferably, L₄ to L₆ can each independently be a single bond, a substituted or unsubstituted C₆₋₂₀ arylene, or a substituted or unsubstituted C₂₋₂₀ heteroarylene containing at least one heteroatom selected from the group consisting of N, O and S.

More preferably, L₄ to L₆ can each independently be a single bond, phenylene, phenylene substituted with four deuteriums, biphenylene, terphenylene, naphthylene, naphthylene substituted with phenyl, carbazolylene, carbazolylene substituted with phenyl, carbazolylene substituted with phenyl substituted with four deuteriums, dibenzofuranylene, dibenzofuranylene substituted with phenyl, dibenzofuranylene substituted with phenyl substituted with four deuteriums, or dimethylfluorenylene.

In addition, L₄ to L₆ can each independently be a single bond or any one group selected from the group consisting of the following groups:

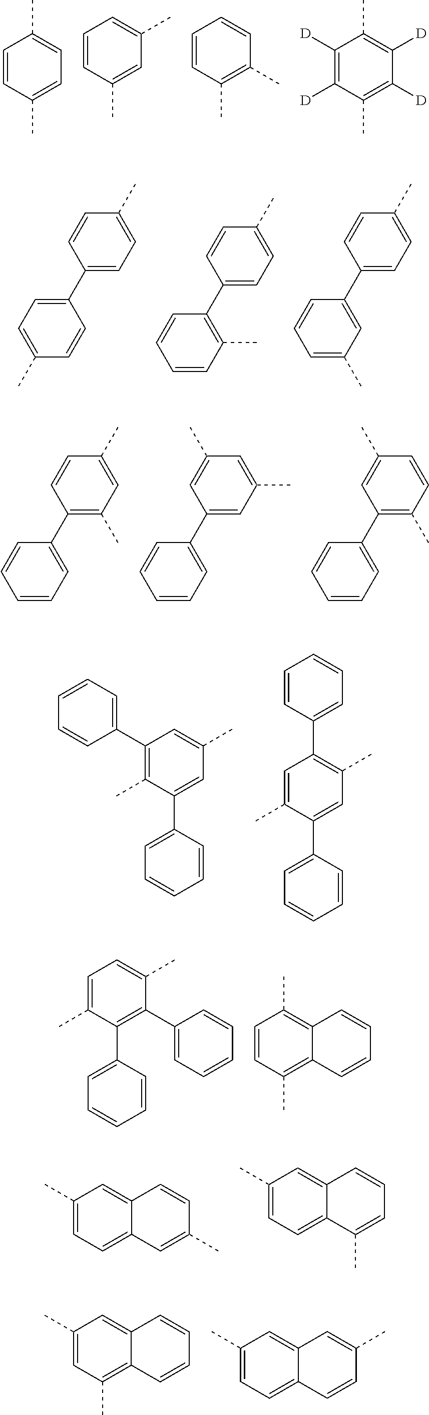

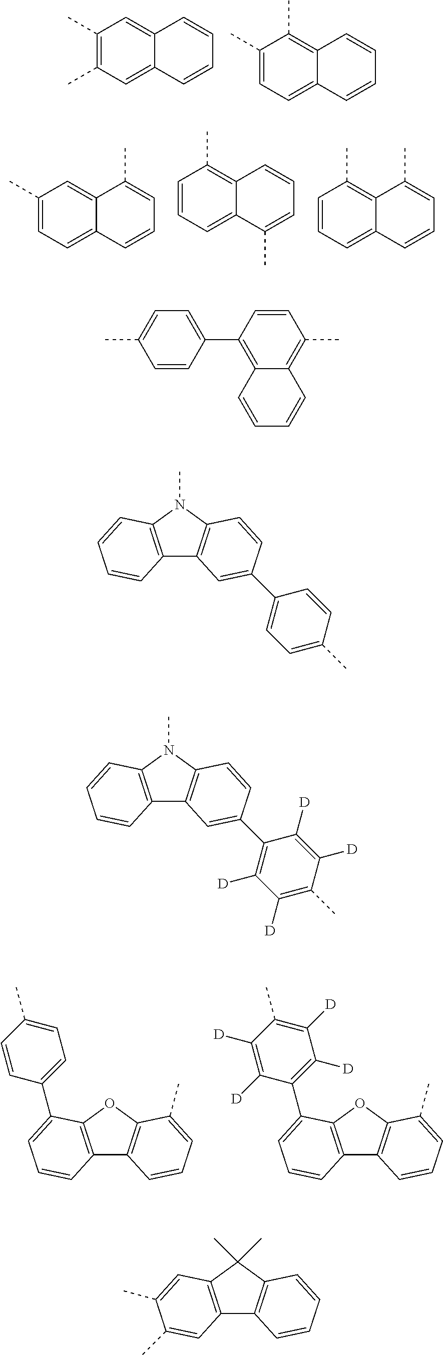

wherein in the above formulae, D represents deuterium.

Preferably, L₄ can be a single bond, and L₅ and L₆ can each independently be a single bond, a substituted or unsubstituted C₆₋₂₀ arylene, or a substituted or unsubstituted C₂₋₂₀ heteroarylene including one or more selected from the group consisting of N, O, and S.

More preferably, L₄ can be a single bond, and L₅ and L₆ can each independently be a single bond, phenylene, phenylene substituted with four deuteriums, biphenylene, naphthylene, naphthylene substituted with phenyl, carbazolylene, carbazolylene substituted with phenyl, carbazolylene substituted with phenyl substituted with four deuteriums, dibenzofuranylene, dibenzofuranylene substituted with phenyl, dibenzofuranylene substituted with phenyl substituted with four deuteriums, or dimethylfluorenylene.

In addition, L₄ can be a single bond, and L₅ and L₆ can each independently be a single bond or any one group selected from the group consisting of the following groups:

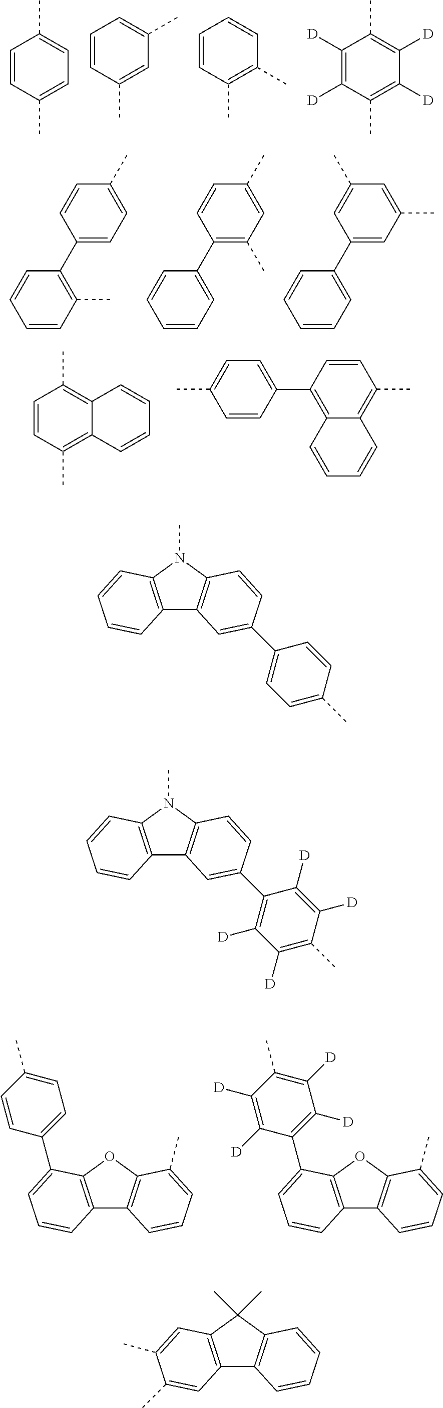

wherein in the above formulae, D represents deuterium.

Preferably, L₇ can be a substituted or unsubstituted C₆₋₂₀ arylene.

More preferably, L₇ can be a substituted or unsubstituted phenylene, a substituted or unsubstituted biphenylene, or a substituted or unsubstituted naphthylene.

In addition, L₇ can be phenylene, phenylene substituted with four deuteriums, biphenylene, or naphthylene.

Preferably, the compound of Chemical Formula 2 can be the following Chemical Formula 2-1 or Chemical Formula 2-2:

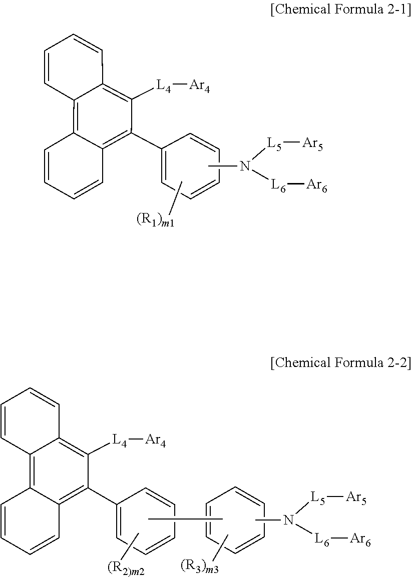

wherein in Chemical Formulae 2-1 and 2-2,
Ar₄ to Ar₆ and L₄ to L₆ are as defined in Chemical Formula 2,
R₁ to R₃ are each independently hydrogen, deuterium, or substituted or unsubstituted C₆₋₆₀ aryl, and
m1 to m3 are each independently an integer of 0 to 4.

Preferably, R₁ to R₃ can each independently be hydrogen, deuterium, or substituted or unsubstituted C₆₋₂₀ aryl.

More preferably, R₁ to R₃ can each independently be hydrogen, or deuterium.

In addition, all hydrogens included in in Chemical Formula 2 and Chemical Formulae 2-1, 2-2 can each independently be replaced with deuterium.

The representative example of the compound of Chemical Formula 2 is as follows:

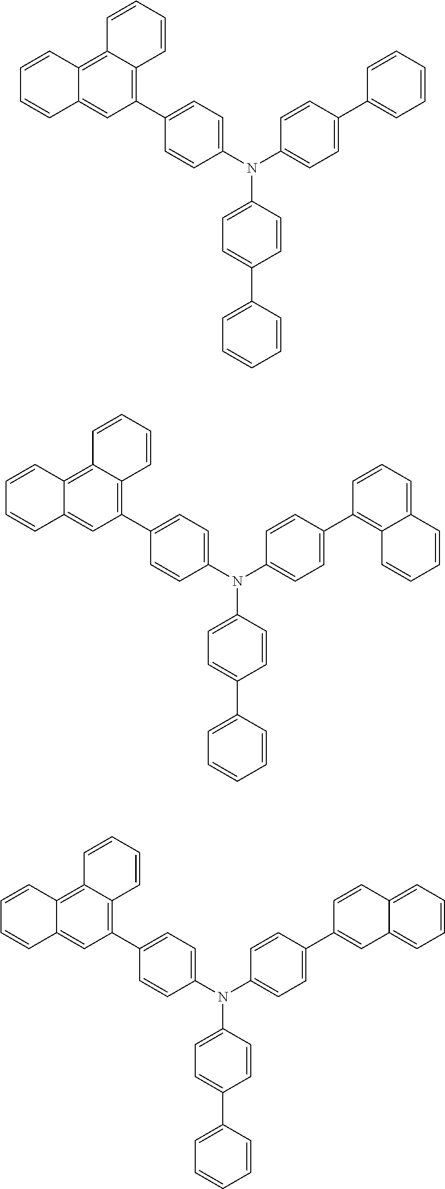 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1]

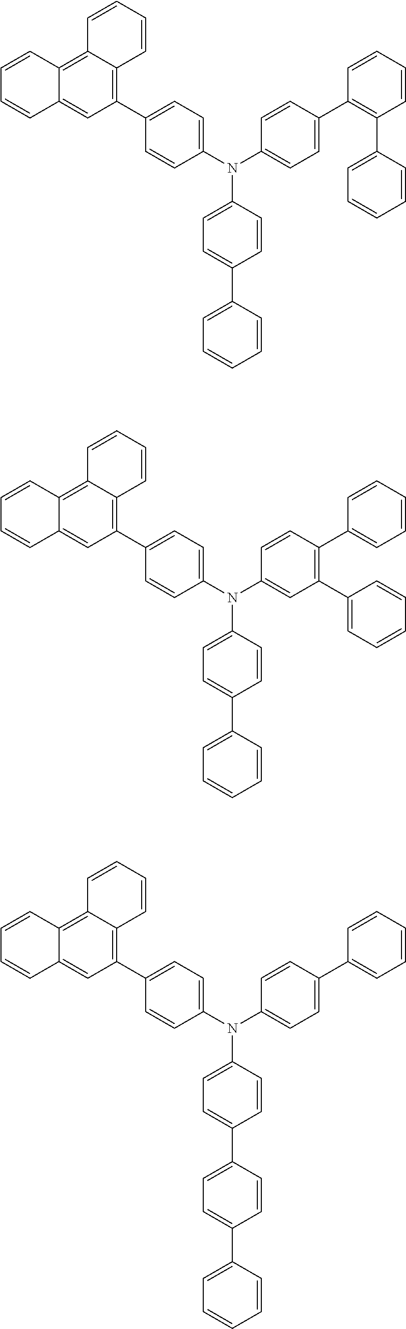 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c(-c4ccccc4)c3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1]

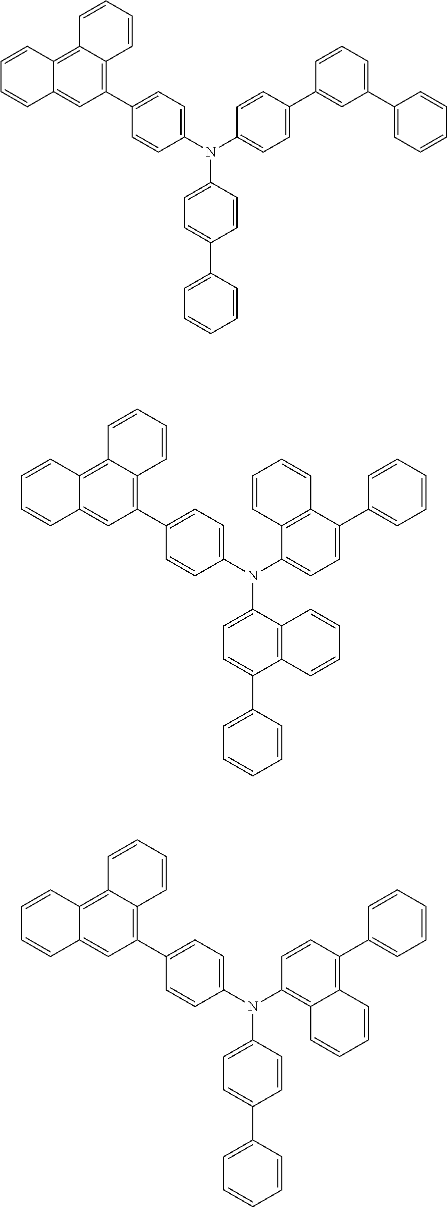 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1]

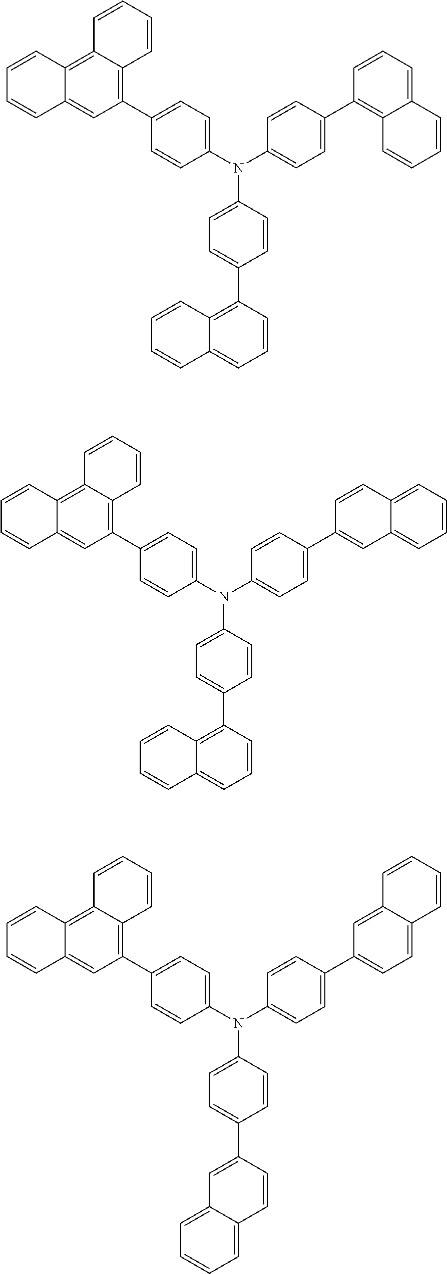 [SMILES: c1ccc2c(-c3ccc(N(c4ccc(-c5cccc6ccccc56)cc4)c4ccc(-c5cc6ccccc6c6ccccc56)cc4)cc3)cccc2c1 ; c1ccc2cc(-c3ccc(N(c4ccc(-c5cccc6ccccc56)cc4)c4ccc(-c5cc6ccccc6c6ccccc56)cc4)cc3)ccc2c1 ; c1ccc2cc(-c3ccc(N(c4ccc(-c5ccc6ccccc6c5)cc4)c4ccc(-c5cc6ccccc6c6ccccc56)cc4)cc3)ccc2c1]

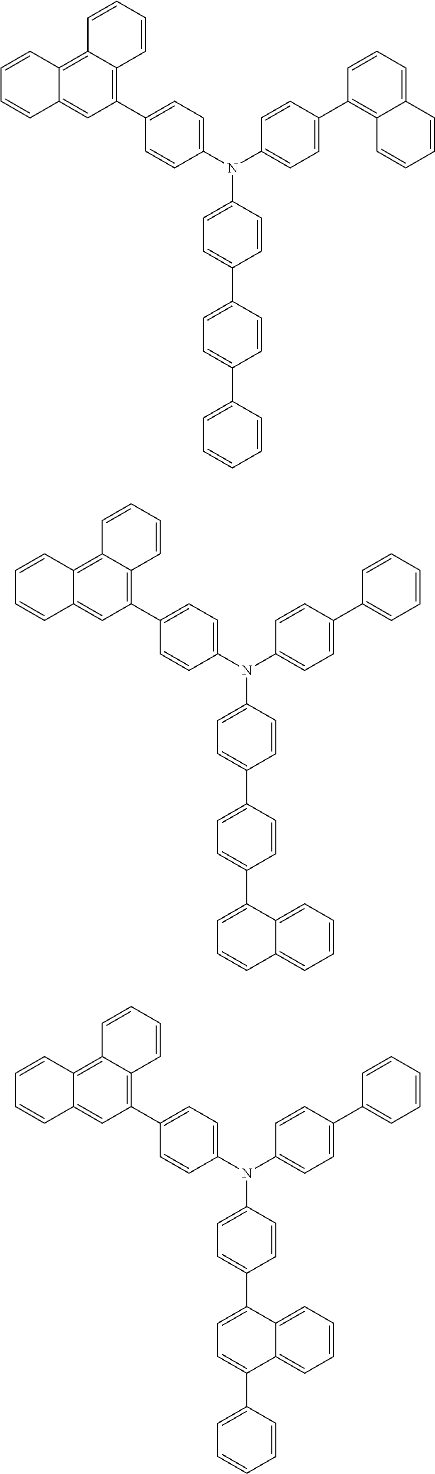 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1 ; c1ccc(-c2ccc(N(c3ccc(-c4ccc(-c5ccccc5)c5ccccc45)cc3)c3ccc(-c4cc5ccccc5c5ccccc45)cc3)cc2)cc1]

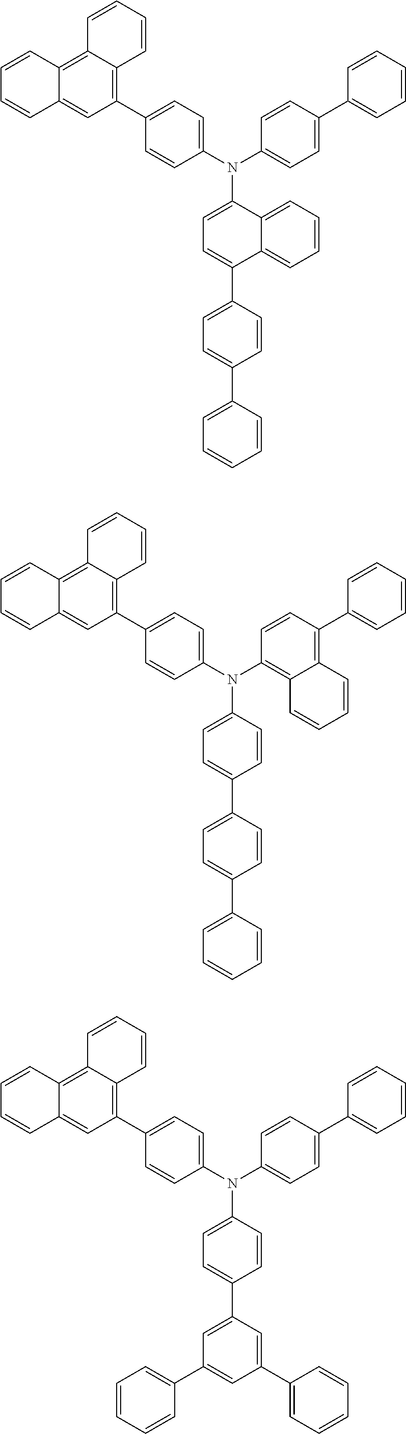 [SMILES: c1ccc(-c2ccc(-c3ccc(N(c4ccc(-c5ccccc5)cc4)c4ccc(-c5cc6ccccc6c6ccccc56)cc4)c4ccccc34)cc2)cc1 ; c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1]

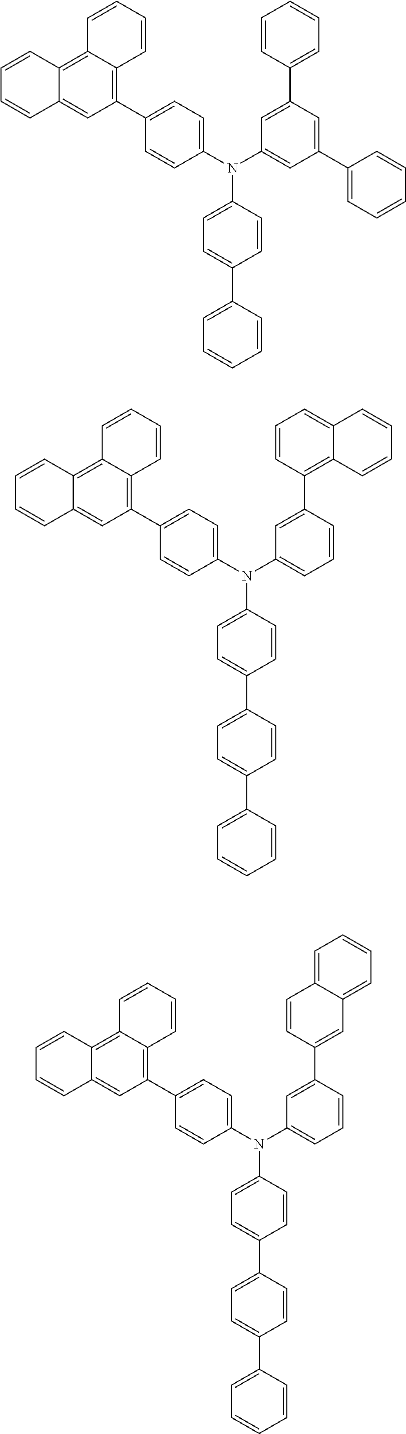 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1]

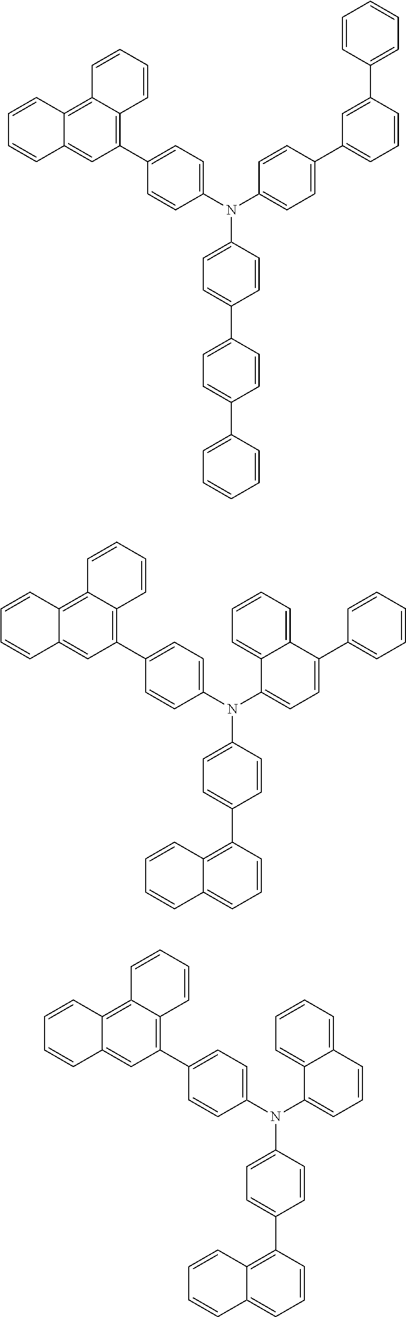

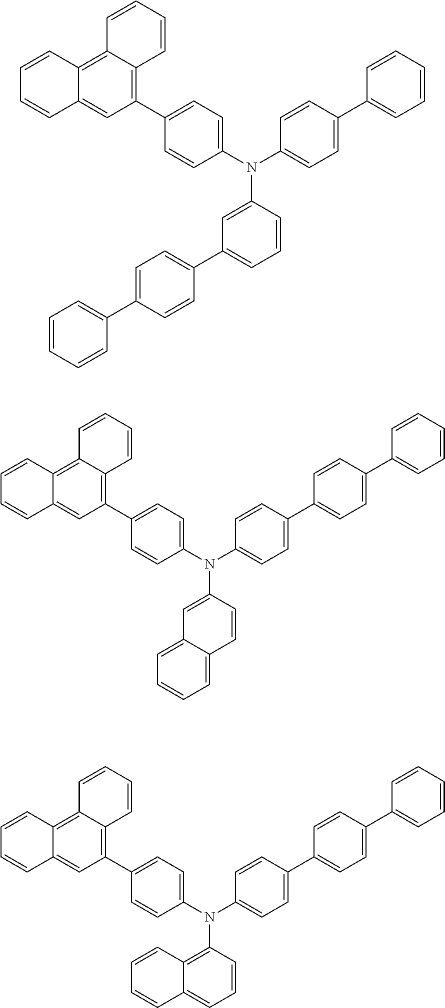 [SMILES: c1ccc(-c2ccc(-c3cccc(N(c4ccc(-c5ccccc5)cc4)c4ccc(-c5cc6ccccc6c6ccccc56)cc4)c3)cc2)cc1 ; c1ccc(-c2ccc(-c3ccc(N(c4ccc(-c5cc6ccccc6c6ccccc56)cc4)c4ccc5ccccc5c4)cc3)cc2)cc1 ; c1ccc(-c2ccc(-c3ccc(N(c4ccc(-c5cc6ccccc6c6ccccc56)cc4)c4cccc5ccccc45)cc3)cc2)cc1]

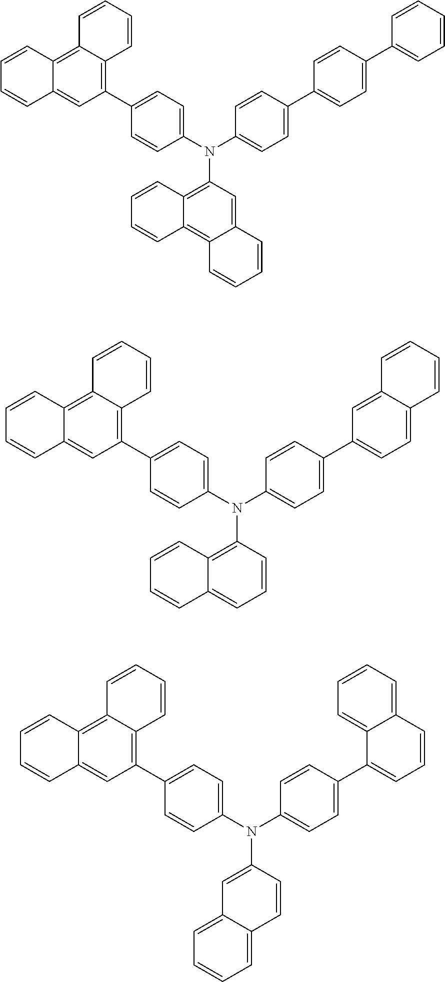 [SMILES: c1ccc(-c2ccc(-c3ccc(N(c4ccc(-c5cc6ccccc6c6ccccc56)cc4)c4cc5ccccc5c5ccccc45)cc3)cc2)cc1 ; c1ccc2cc(-c3ccc(N(c4ccc(-c5cc6ccccc6c6ccccc56)cc4)c4cccc5ccccc45)cc3)ccc2c1 ; c1ccc2cc(N(c3ccc(-c4cccc5ccccc45)cc3)c3ccc(-c4cc5ccccc5c5ccccc45)cc3)ccc2c1]

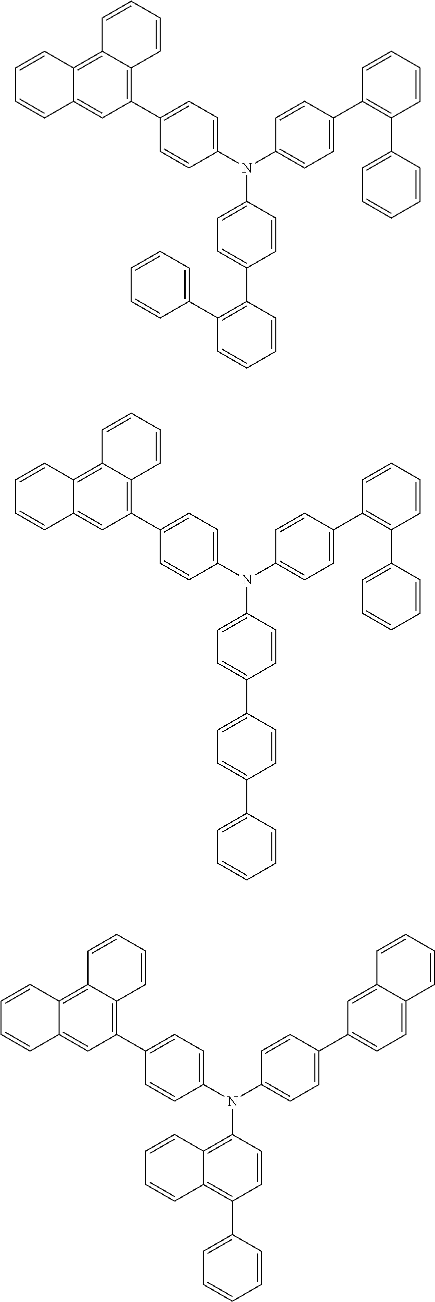 [SMILES: c1ccc(-c2ccccc2-c2ccc(N(c3ccc(-c4ccccc4-c4ccccc4)cc3)c3ccc(-c4cc5ccccc5c5ccccc45)cc3)cc2)cc1 ; c1ccc(-c2ccc(-c3ccc(N(c4ccc(-c5ccccc5-c5ccccc5)cc4)c4ccc(-c5cc6ccccc6c6ccccc56)cc4)cc3)cc2)cc1 ; c1ccc(-c2ccc(N(c3ccc(-c4ccc5ccccc5c4)cc3)c3ccc(-c4cc5ccccc5c5ccccc45)cc3)c3ccccc23)cc1]

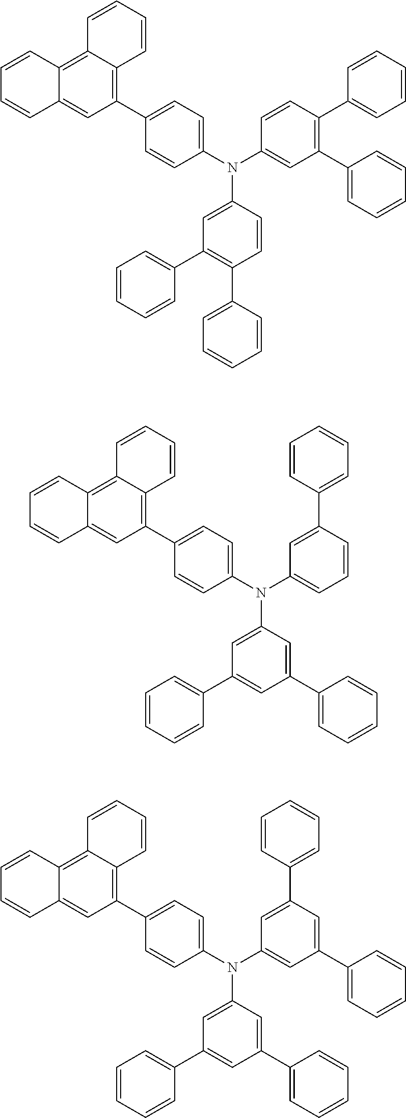 [SMILES: c1ccc(-c2ccc(N(c3ccc(-c4cc5ccccc5c5ccccc45)cc3)c3ccc(-c4ccccc4)c(-c4ccccc4)c3)cc2-c2ccccc2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2cc(-c3ccccc3)cc(N(c3ccc(-c4cc5ccccc5c5ccccc45)cc3)c3cc(-c4ccccc4)cc(-c4ccccc4)c3)c2)cc1]

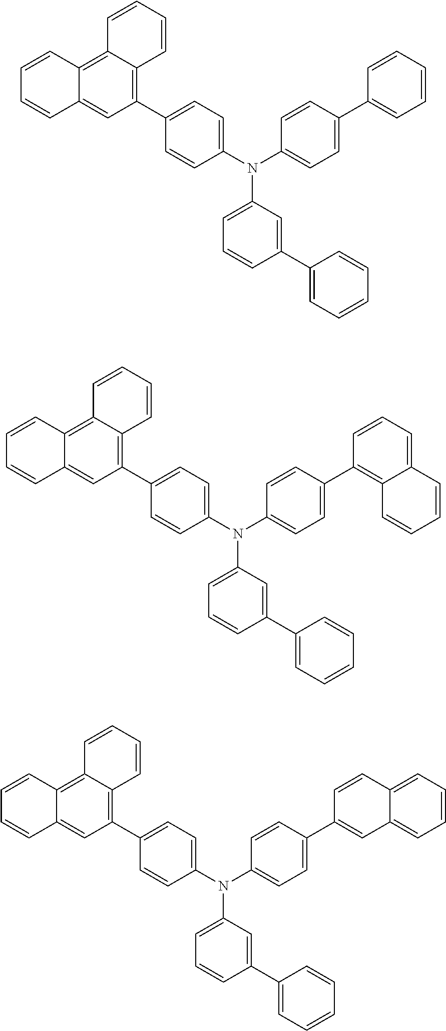 [SMILES: c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1 ; c1ccc(-c2cccc(N(c3ccc(-c4ccc5ccccc5c4)cc3)c3ccc(-c4cc5ccccc5c5ccccc45)cc3)c2)cc1]

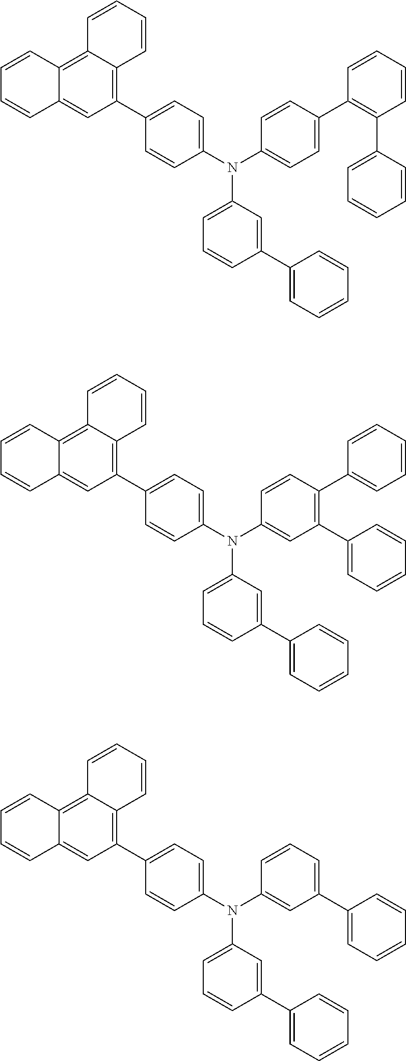 [SMILES: c1ccc(-c2cccc(N(c3ccc(-c4ccccc4-c4ccccc4)cc3)c3ccc(-c4cc5ccccc5c5ccccc45)cc3)c2)cc1 ; c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1]

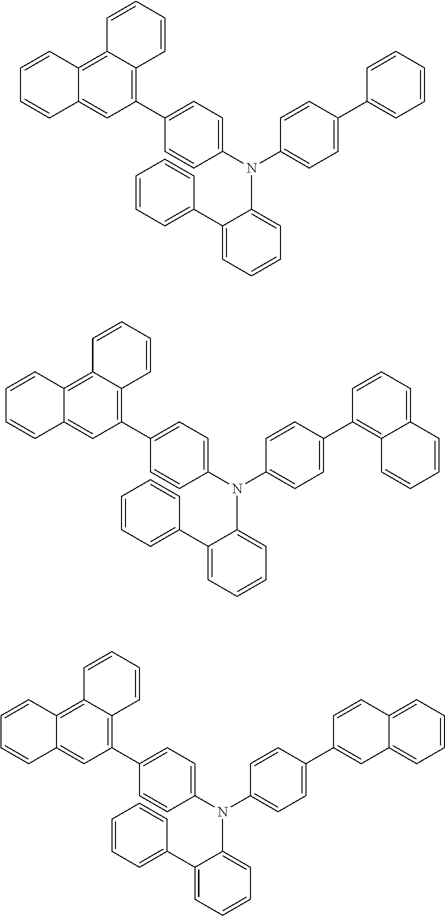 [SMILES: c1ccc(-c2ccc(N(c3ccc(-c4cc5ccccc5c5ccccc45)cc3)c3ccccc3-c3ccccc3)cc2)cc1 ; c1ccc(-c2ccccc2N(c2ccc(-c3cccc4ccccc34)cc2)c2ccc(-c3cc4ccccc4c4ccccc34)cc2)cc1 ; c1ccc(-c2ccccc2N(c2ccc(-c3ccc4ccccc4c3)cc2)c2ccc(-c3cc4ccccc4c4ccccc34)cc2)cc1]

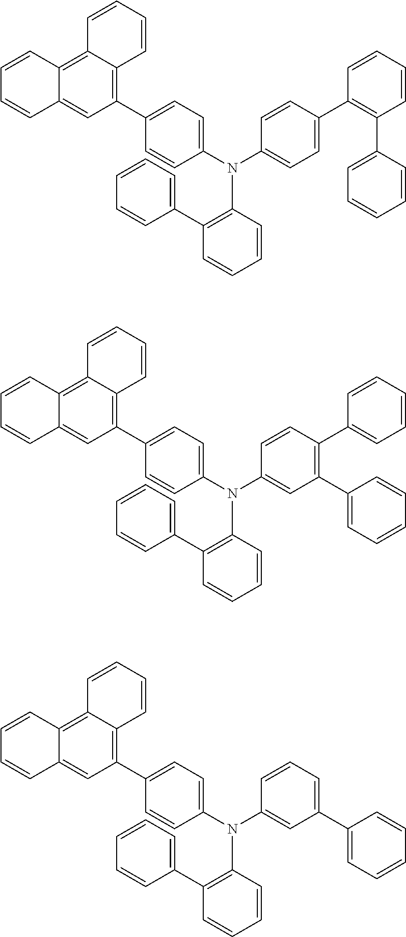 [SMILES: c1ccc(-c2ccccc2-c2ccc(N(c3ccc(-c4cc5ccccc5c5ccccc45)cc3)c3ccccc3-c3ccccc3)cc2)cc1 ; c1ccc(-c2ccc(N(c3ccc(-c4cc5ccccc5c5ccccc45)cc3)c3ccccc3-c3ccccc3)cc2-c2ccccc2)cc1 ; c1ccc(-c2cccc(N(c3ccc(-c4cc5ccccc5c5ccccc45)cc3)c3ccccc3-c3ccccc3)c2)cc1]

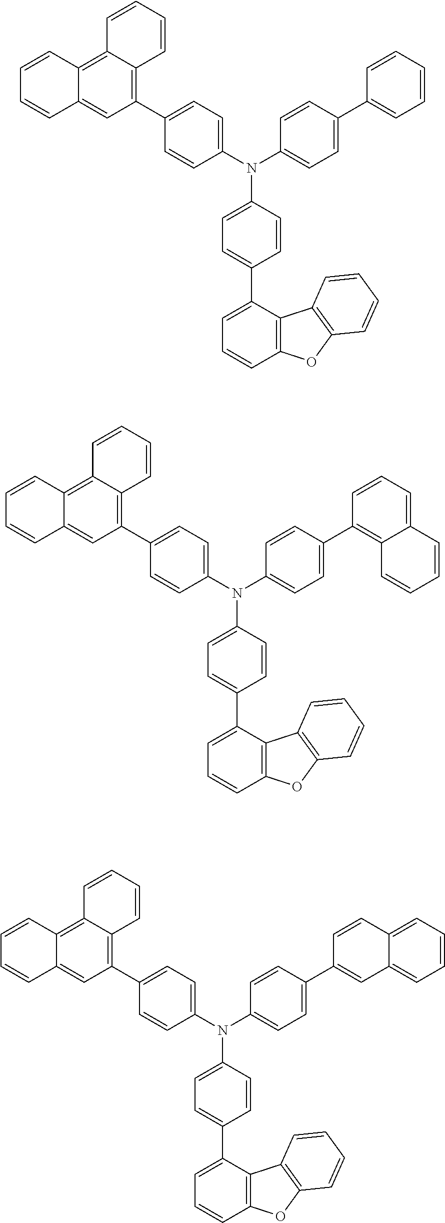 [SMILES: c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1 ; c1ccc2cc(-c3ccc(N(c4ccc(-c5cc6ccccc6c6ccccc56)cc4)c4ccc(-c5cccc6oc7ccccc7c56)cc4)cc3)ccc2c1]

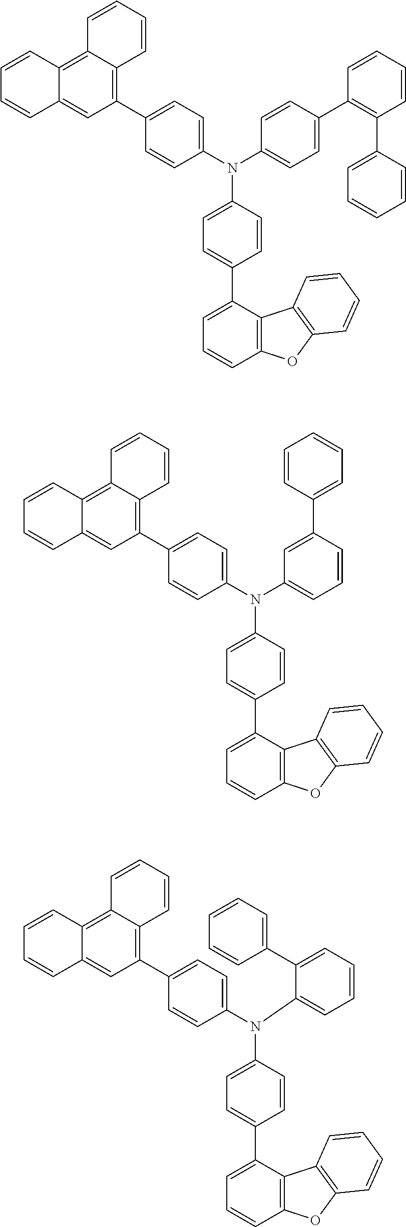 [SMILES: c1ccc(-c2ccccc2-c2ccc(N(c3ccc(-c4cc5ccccc5c5ccccc45)cc3)c3ccc(-c4cccc5oc6ccccc6c45)cc3)cc2)cc1 ; c1ccc(-c2cccc(N(c3ccc(-c4cc5ccccc5c5ccccc45)cc3)c3ccc(-c4cccc5oc6ccccc6c45)cc3)c2)cc1 ; c1ccc(-c2ccccc2N(c2ccc(-c3cc4ccccc4c4ccccc34)cc2)c2ccc(-c3cccc4oc5ccccc5c34)cc2)cc1]

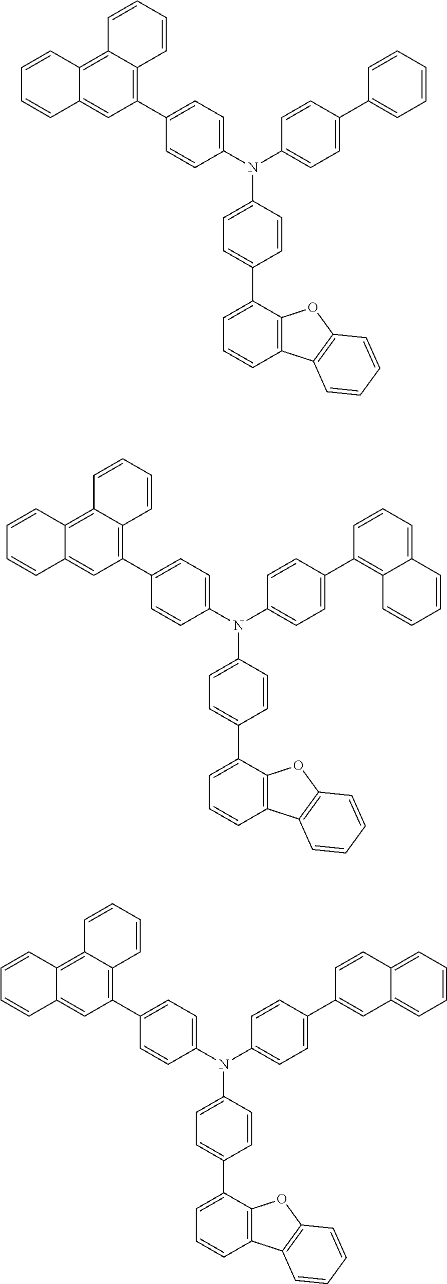 [SMILES: c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1 ; c1ccc2cc(-c3ccc(N(c4ccc(-c5cc6ccccc6c6ccccc56)cc4)c4ccc(-c5cccc6c5oc5ccccc56)cc4)cc3)ccc2c1]

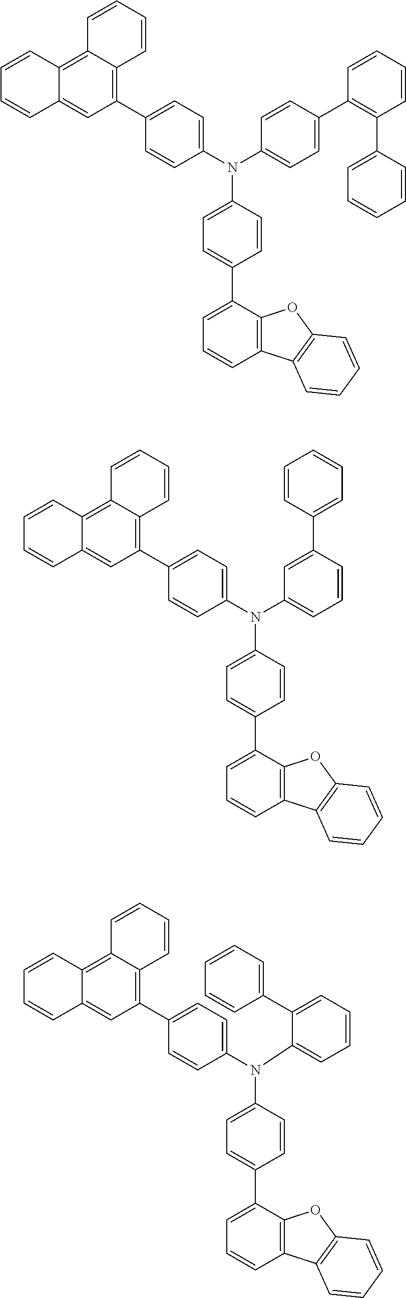 [SMILES: c1ccc(-c2ccccc2-c2ccc(N(c3ccc(-c4cc5ccccc5c5ccccc45)cc3)c3ccc(-c4cccc5c4oc4ccccc45)cc3)cc2)cc1 ; c1ccc(-c2cccc(N(c3ccc(-c4cc5ccccc5c5ccccc45)cc3)c3ccc(-c4cccc5c4oc4ccccc45)cc3)c2)cc1 ; c1ccc(-c2ccccc2N(c2ccc(-c3cc4ccccc4c4ccccc34)cc2)c2ccc(-c3cccc4c3oc3ccccc34)cc2)cc1]

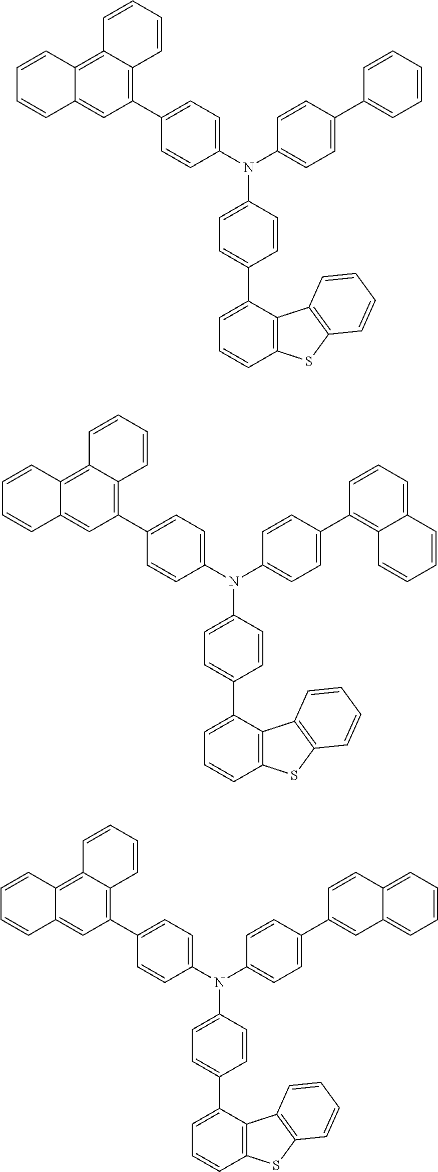 [SMILES: c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1 ; c1ccc2cc(-c3ccc(N(c4ccc(-c5cc6ccccc6c6ccccc56)cc4)c4ccc(-c5cccc6sc7ccccc7c56)cc4)cc3)ccc2c1]

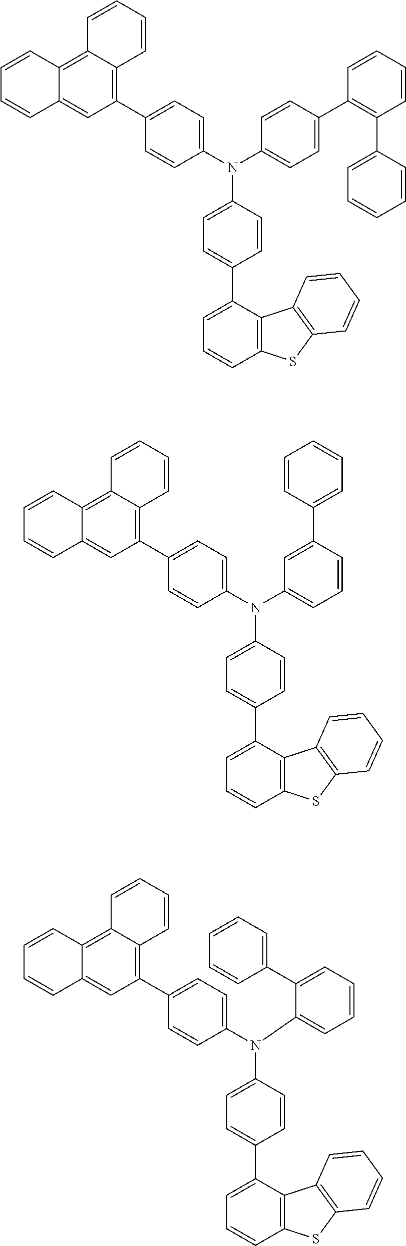 [SMILES: c1ccc(-c2ccccc2-c2ccc(N(c3ccc(-c4cc5ccccc5c5ccccc45)cc3)c3ccc(-c4cccc5sc6ccccc6c45)cc3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1 ; c1ccc(-c2ccccc2N(c2ccc(-c3cc4ccccc4c4ccccc34)cc2)c2ccc(-c3cccc4sc5ccccc5c34)cc2)cc1]

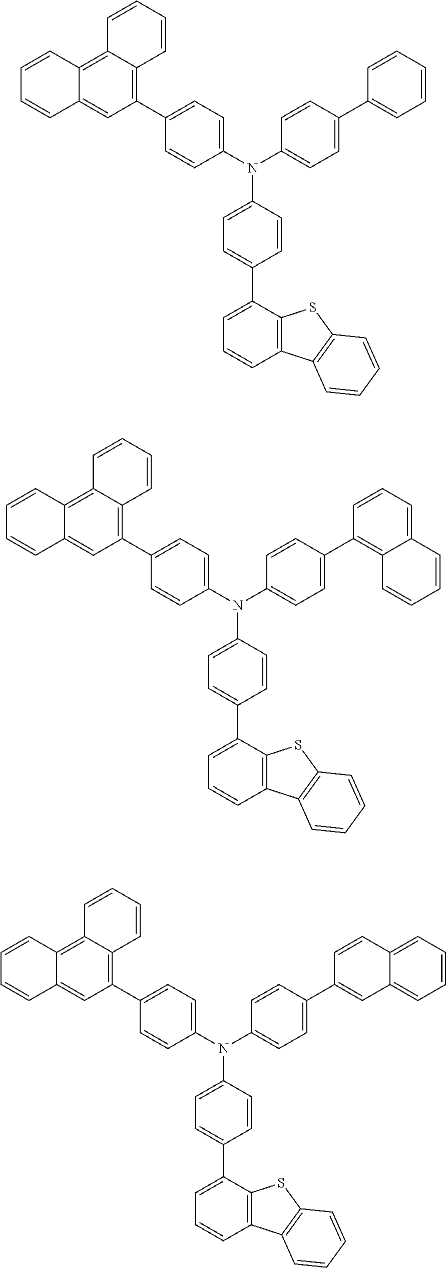 [SMILES: c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1 ; c1ccc2cc(-c3ccc(N(c4ccc(-c5cc6ccccc6c6ccccc56)cc4)c4ccc(-c5cccc6c5sc5ccccc56)cc4)cc3)ccc2c1]

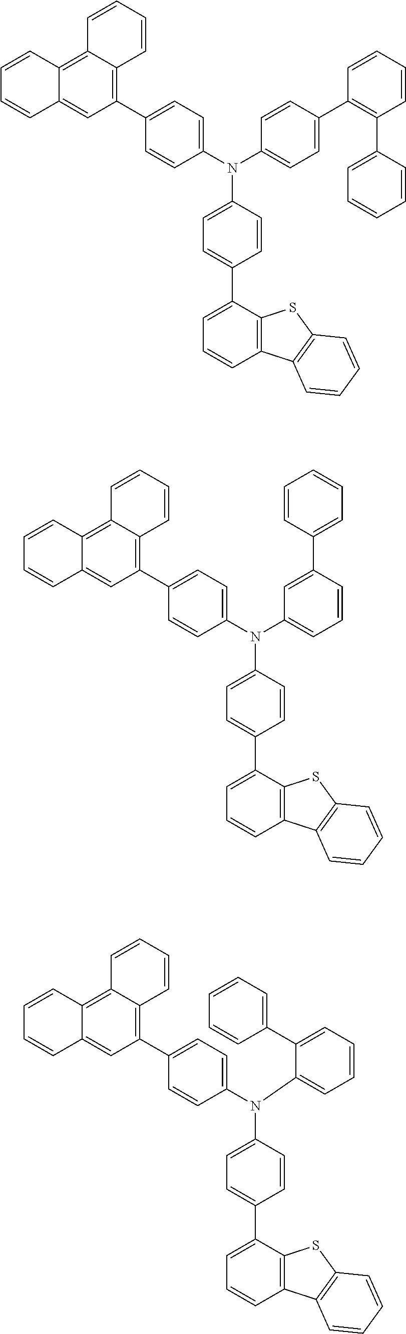 [SMILES: c1ccc(-c2ccccc2-c2ccc(N(c3ccc(-c4cc5ccccc5c5ccccc45)cc3)c3ccc(-c4cccc5c4sc4ccccc45)cc3)cc2)cc1 ; c1ccc(-c2cccc(N(c3ccc(-c4cc5ccccc5c5ccccc45)cc3)c3ccc(-c4cccc5c4sc4ccccc45)cc3)c2)cc1 ; c1ccc(-c2ccccc2N(c2ccc(-c3cc4ccccc4c4ccccc34)cc2)c2ccc(-c3cccc4c3sc3ccccc34)cc2)cc1]

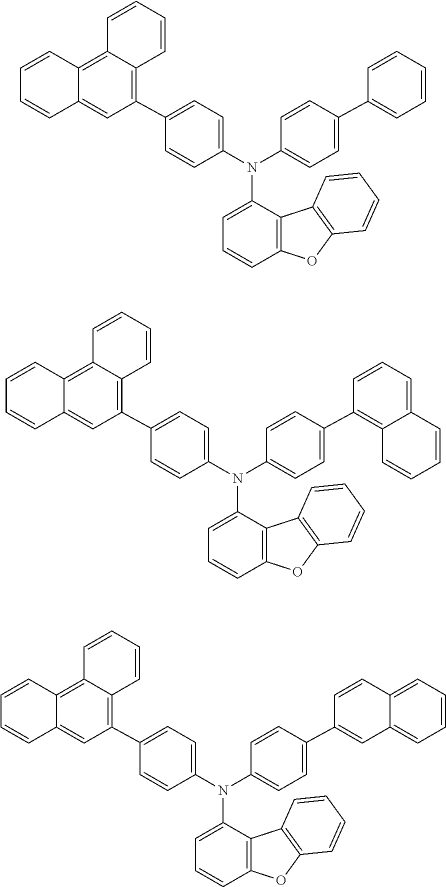 [SMILES: c1ccc(-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)cc3)cccc2c1 ; c1ccc2cc(-c3ccc(N(c4ccc(-c5cc6ccccc6c6ccccc56)cc4)c4cccc5oc6ccccc6c45)cc3)ccc2c1]

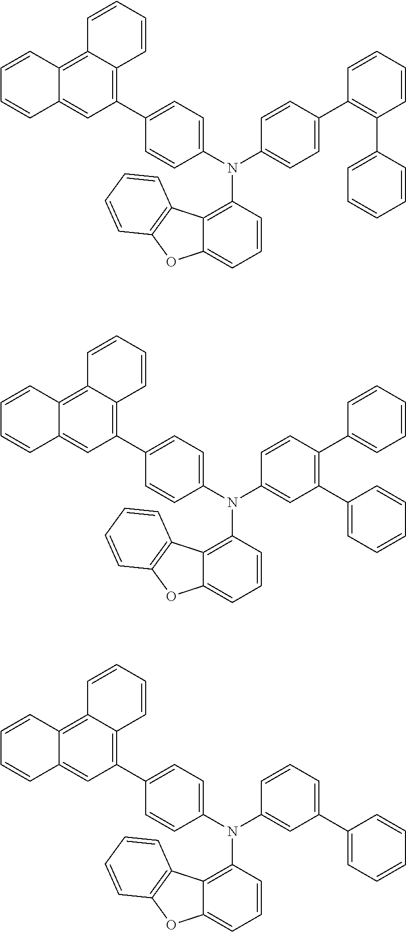 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2-c2ccccc2)cc1 ; c1ccc(-c2cccc(N(c3ccc(-c4cc5ccccc5c5ccccc45)cc3)c3cccc4oc5ccccc5c34)c2)cc1]

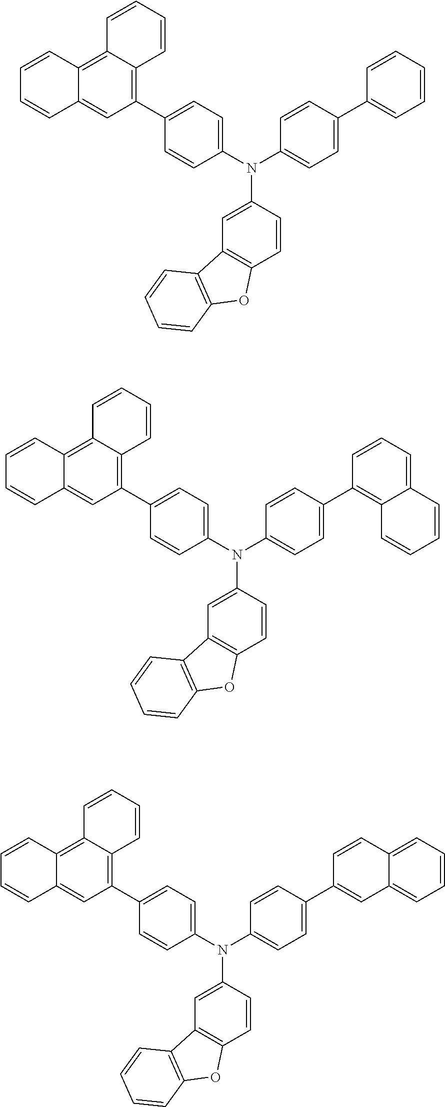 [SMILES: c1ccc(-c2ccc(N(c3ccc(-c4cc5ccccc5c5ccccc45)cc3)c3ccc4oc5ccccc5c4c3)cc2)cc1 ; c1ccc2c(-c3ccc(N(c4ccc(-c5cc6ccccc6c6ccccc56)cc4)c4ccc5oc6ccccc6c5c4)cc3)cccc2c1 ; c1ccc2cc(-c3ccc(N(c4ccc(-c5cc6ccccc6c6ccccc56)cc4)c4ccc5oc6ccccc6c5c4)cc3)ccc2c1]

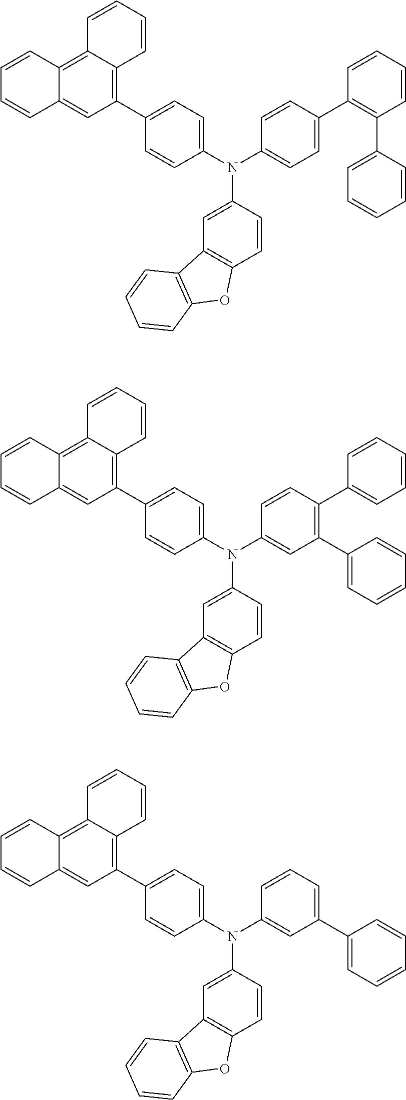 [SMILES: c1ccc(-c2ccccc2-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4oc5ccccc5c4c3)cc2-c2ccccc2)cc1 ; c1ccc(-c2cccc(N(c3ccc(-c4cc5ccccc5c5ccccc45)cc3)c3ccc4oc5ccccc5c4c3)c2)cc1]

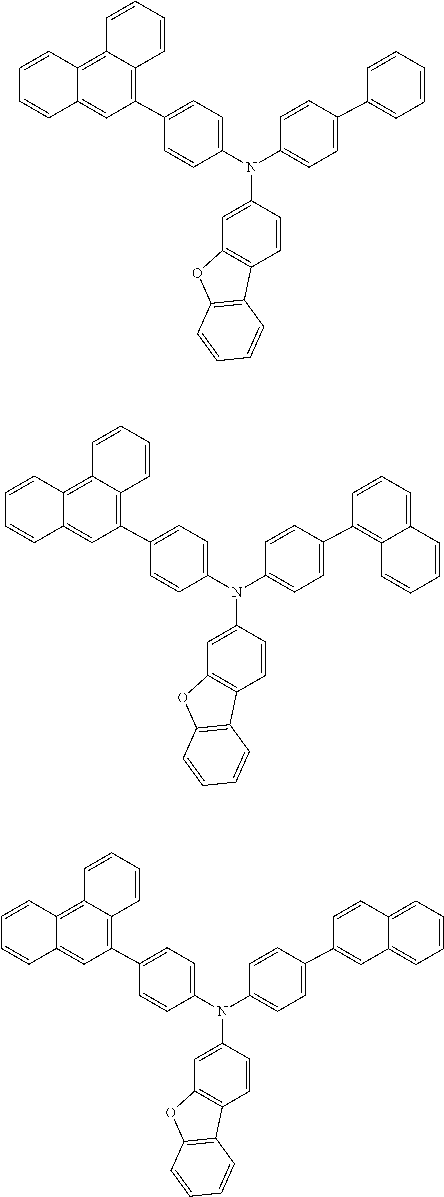 [SMILES: c1ccc(-c2ccc(N(c3ccc(-c4cc5ccccc5c5ccccc45)cc3)c3ccc4c(c3)oc3ccccc34)cc2)cc1 ; c1ccc2c(-c3ccc(N(c4ccc(-c5cc6ccccc6c6ccccc56)cc4)c4ccc5c(c4)oc4ccccc45)cc3)cccc2c1 ; c1ccc2cc(-c3ccc(N(c4ccc(-c5cc6ccccc6c6ccccc56)cc4)c4ccc5c(c4)oc4ccccc45)cc3)ccc2c1]

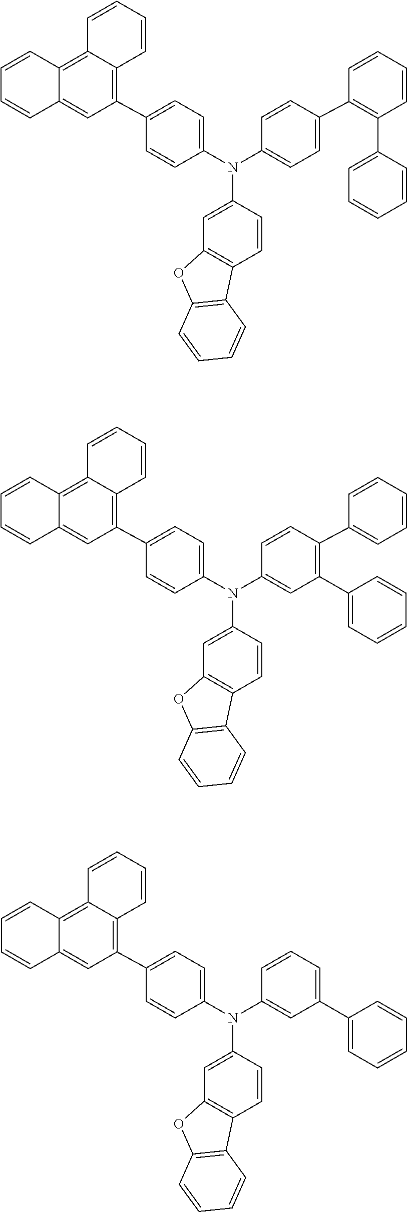 [SMILES: c1ccc(-c2ccccc2-c2ccc(N(c3ccc(-c4cc5ccccc5c5ccccc45)cc3)c3ccc4c(c3)oc3ccccc34)cc2)cc1 ; c1ccc(-c2ccc(N(c3ccc(-c4cc5ccccc5c5ccccc45)cc3)c3ccc4c(c3)oc3ccccc34)cc2-c2ccccc2)cc1 ; c1ccc(-c2cccc(N(c3ccc(-c4cc5ccccc5c5ccccc45)cc3)c3ccc4c(c3)oc3ccccc34)c2)cc1]

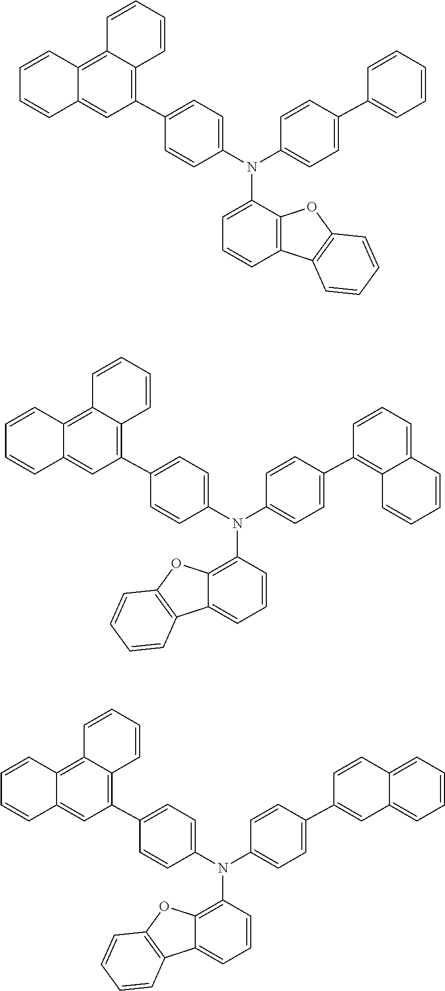 [SMILES: c1ccc(-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)cc3)cccc2c1 ; c1ccc2cc(-c3ccc(N(c4ccc(-c5cc6ccccc6c6ccccc56)cc4)c4cccc5c4oc4ccccc45)cc3)ccc2c1]

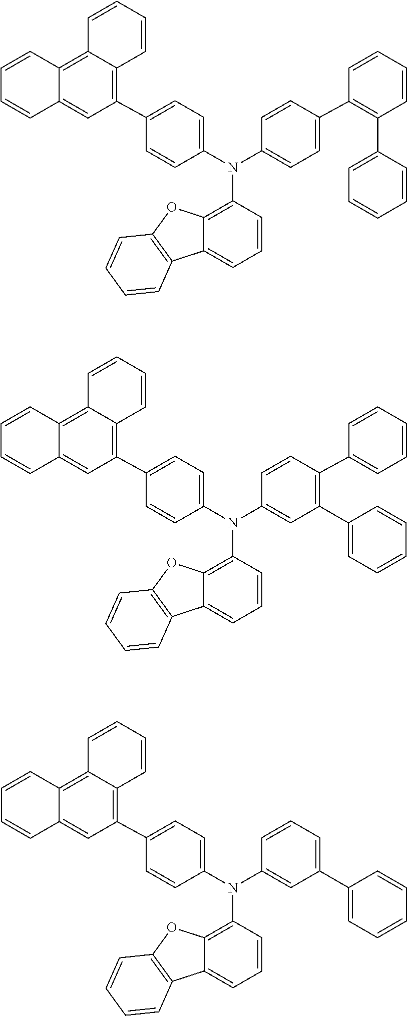 [SMILES: c1ccc(-c2ccccc2-c2ccc(N(c3ccc(-c4cc5ccccc5c5ccccc45)cc3)c3cccc4c3oc3ccccc34)cc2)cc1 ; c1ccc(-c2ccc(N(c3ccc(-c4cc5ccccc5c5ccccc45)cc3)c3cccc4c3oc3ccccc34)cc2-c2ccccc2)cc1 ; c1ccc(-c2cccc(N(c3ccc(-c4cc5ccccc5c5ccccc45)cc3)c3cccc4c3oc3ccccc34)c2)cc1]

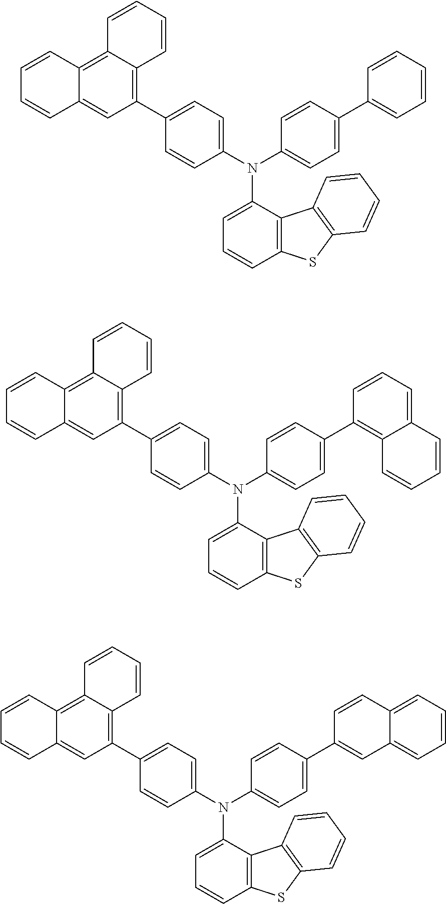 [SMILES: c1ccc(-c2ccc(N(c3ccc(-c4cc5ccccc5c5ccccc45)cc3)c3cccc4sc5ccccc5c34)cc2)cc1 ; c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1]

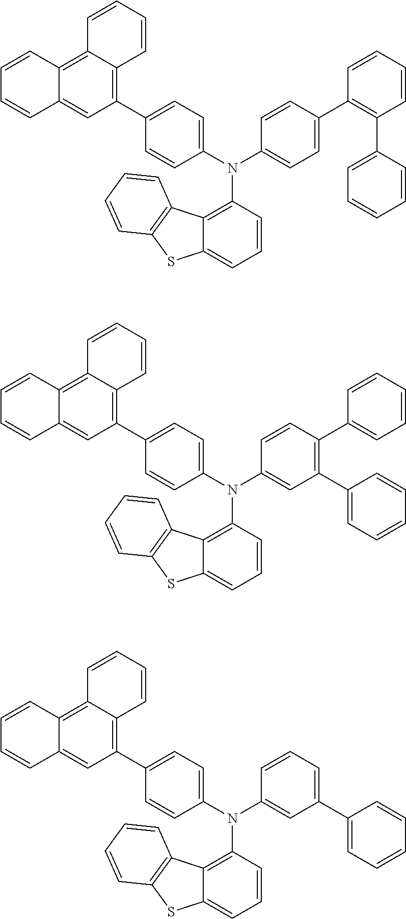 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)cc2-c2ccccc2)cc1 ; c1ccc(-c2cccc(N(c3ccc(-c4cc5ccccc5c5ccccc45)cc3)c3cccc4sc5ccccc5c34)c2)cc1]

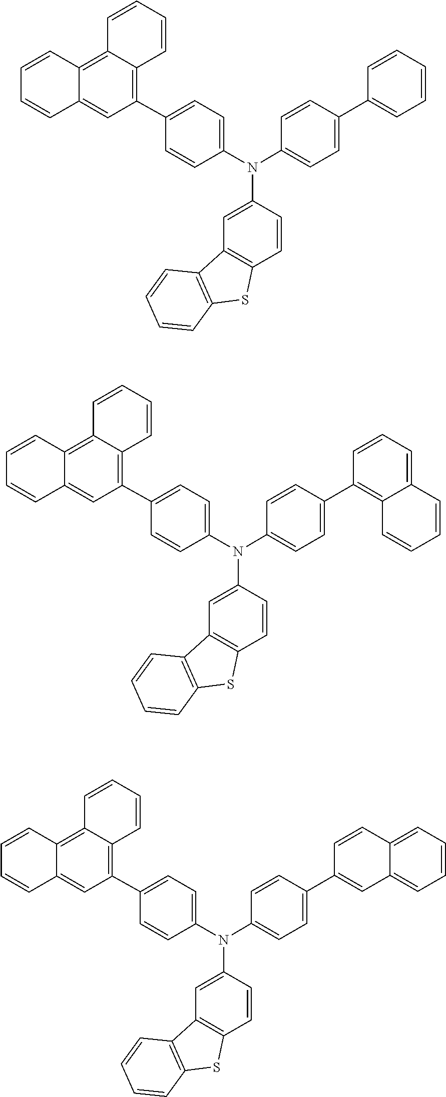 [SMILES: c1ccc(-c2ccc(N(c3ccc(-c4cc5ccccc5c5ccccc45)cc3)c3ccc4sc5ccccc5c4c3)cc2)cc1 ; c1ccc2c(-c3ccc(N(c4ccc(-c5cc6ccccc6c6ccccc56)cc4)c4ccc5sc6ccccc6c5c4)cc3)cccc2c1 ; c1ccc2cc(-c3ccc(N(c4ccc(-c5cc6ccccc6c6ccccc56)cc4)c4ccc5sc6ccccc6c5c4)cc3)ccc2c1]

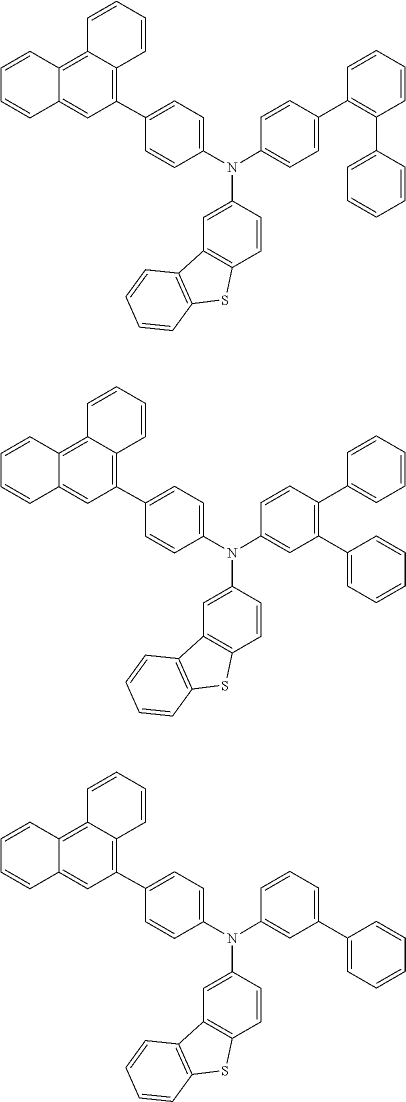 [SMILES: c1ccc(-c2ccccc2-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4sc5ccccc5c4c3)cc2-c2ccccc2)cc1 ; c1ccc(-c2cccc(N(c3ccc(-c4cc5ccccc5c5ccccc45)cc3)c3ccc4sc5ccccc5c4c3)c2)cc1]

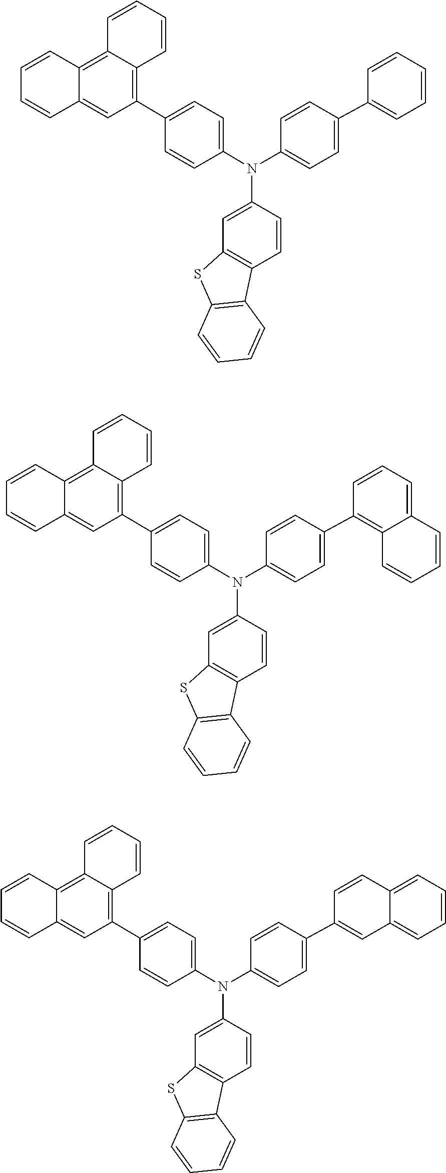 [SMILES: c1ccc(-c2ccc(N(c3ccc(-c4cc5ccccc5c5ccccc45)cc3)c3ccc4c(c3)sc3ccccc34)cc2)cc1 ; c1ccc2c(-c3ccc(N(c4ccc(-c5cc6ccccc6c6ccccc56)cc4)c4ccc5c(c4)sc4ccccc45)cc3)cccc2c1 ; c1ccc2cc(-c3ccc(N(c4ccc(-c5cc6ccccc6c6ccccc56)cc4)c4ccc5c(c4)sc4ccccc45)cc3)ccc2c1]

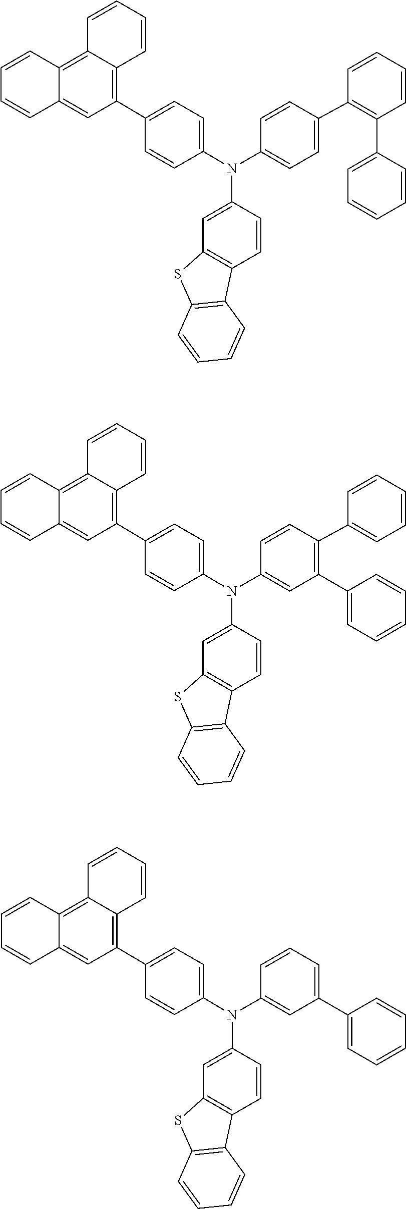 [SMILES: c1ccc(-c2ccccc2-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2-c2ccccc2)cc1 ; c1ccc(-c2cccc(N(c3ccc(-c4cc5ccccc5c5ccccc45)cc3)c3ccc4c(c3)sc3ccccc34)c2)cc1]

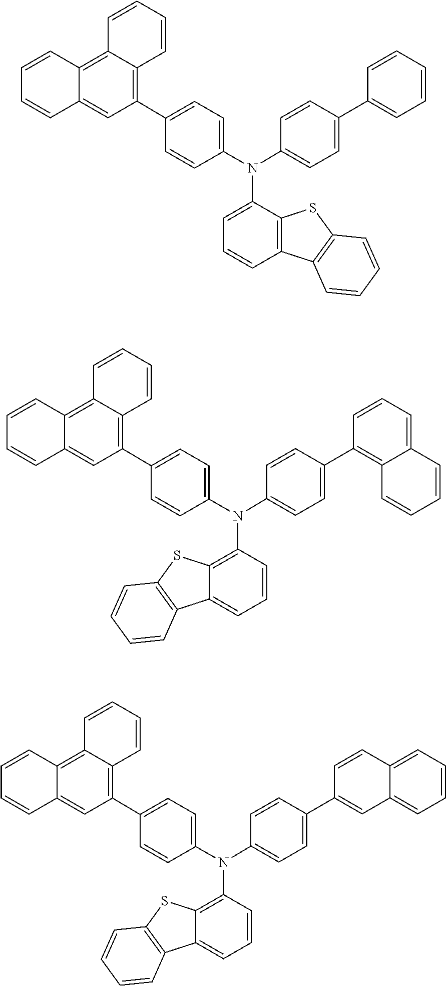 [SMILES: c1ccc(-c2ccc(N(c3ccc(-c4cc5ccccc5c5ccccc45)cc3)c3cccc4c3sc3ccccc34)cc2)cc1 ; c1ccc2c(-c3ccc(N(c4ccc(-c5cc6ccccc6c6ccccc56)cc4)c4cccc5c4sc4ccccc45)cc3)cccc2c1 ; c1ccc2cc(-c3ccc(N(c4ccc(-c5cc6ccccc6c6ccccc56)cc4)c4cccc5c4sc4ccccc45)cc3)ccc2c1]

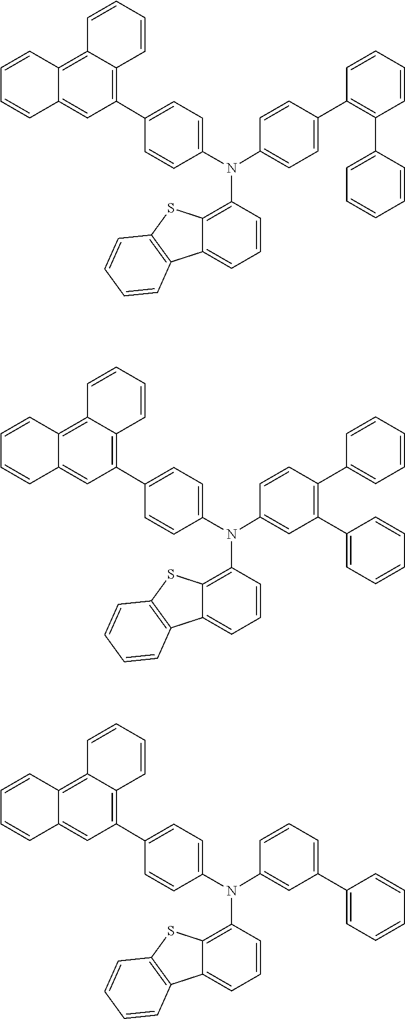 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)cc2-c2ccccc2)cc1 ; c1ccc(-c2cccc(N(c3ccc(-c4cc5ccccc5c5ccccc45)cc3)c3cccc4c3sc3ccccc34)c2)cc1]

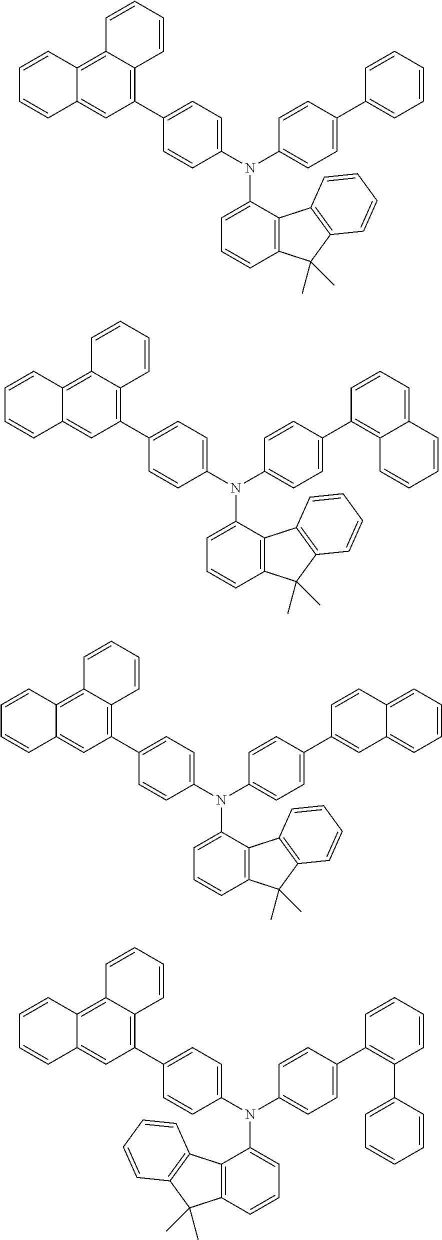 [SMILES: CC1(C)c2ccccc2-c2c(N(c3ccc(-c4ccccc4)cc3)c3ccc(-c4cc5ccccc5c5ccccc45)cc3)cccc21 ; CC1(C)c2ccccc2-c2c(N(c3ccc(-c4cccc5ccccc45)cc3)c3ccc(-c4cc5ccccc5c5ccccc45)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21 ; CC1(C)c2ccccc2-c2c(N(c3ccc(-c4ccccc4-c4ccccc4)cc3)c3ccc(-c4cc5ccccc5c5ccccc45)cc3)cccc21]

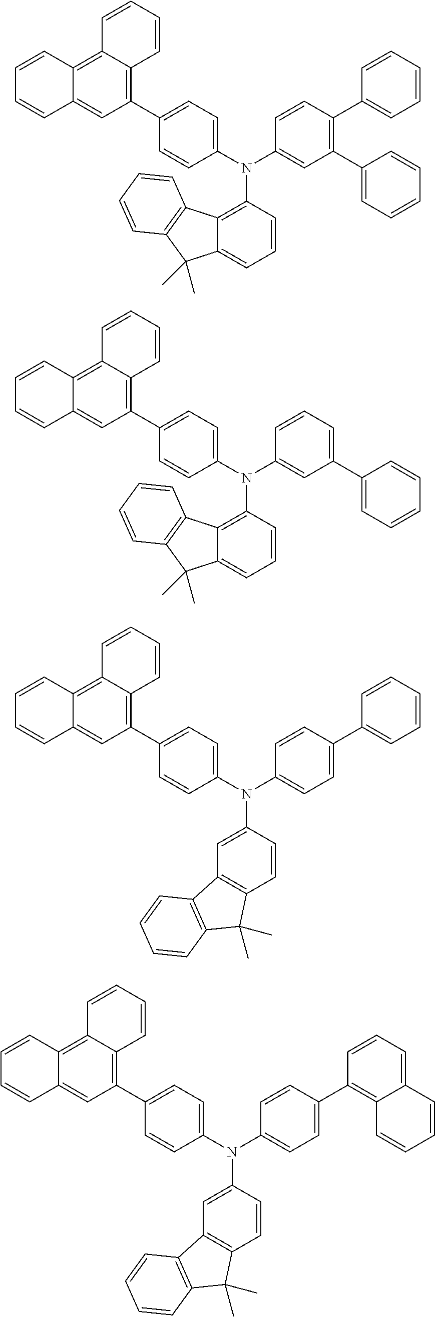

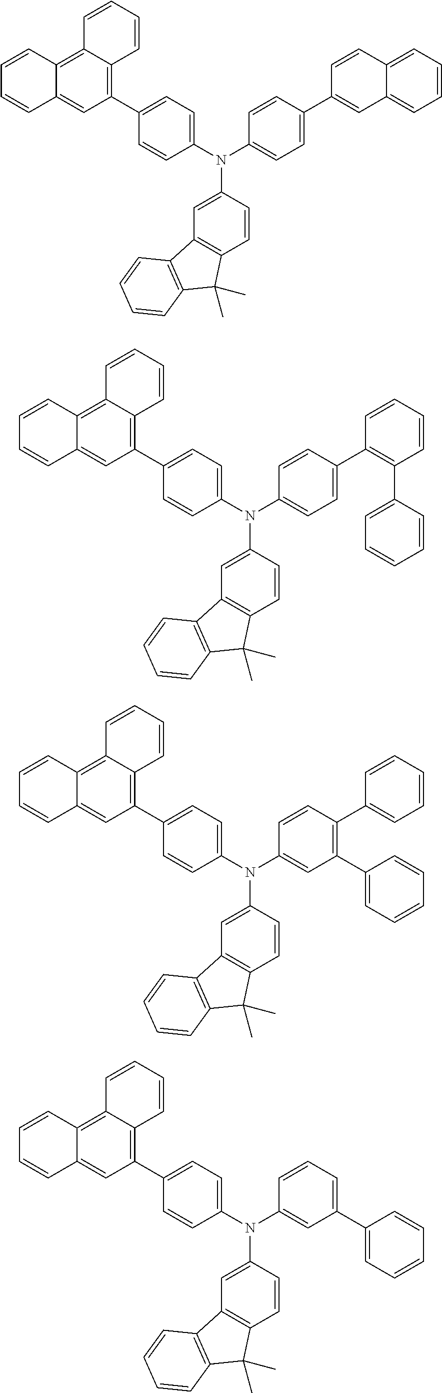

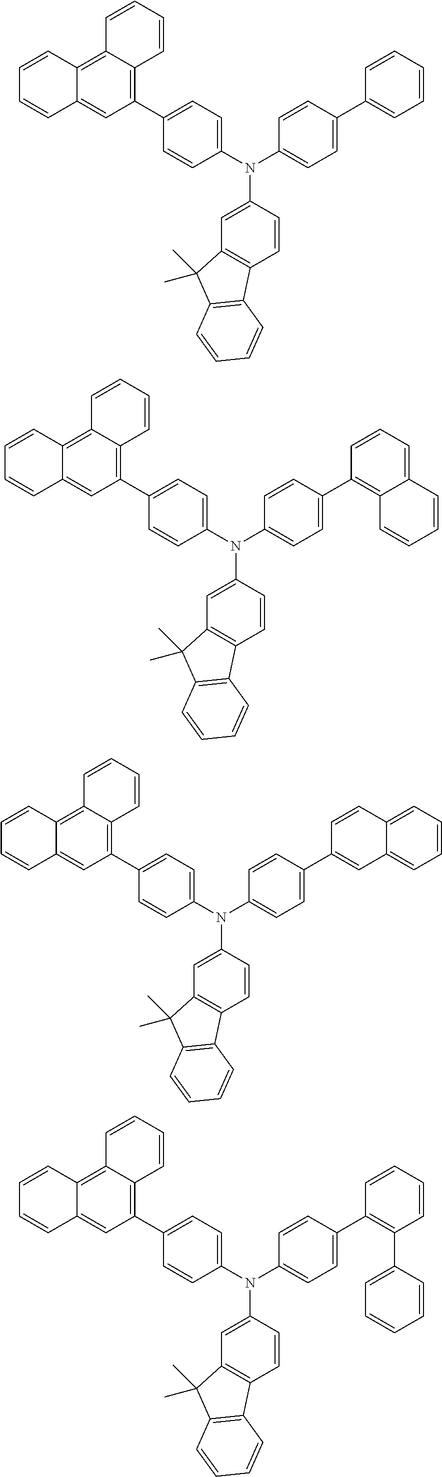

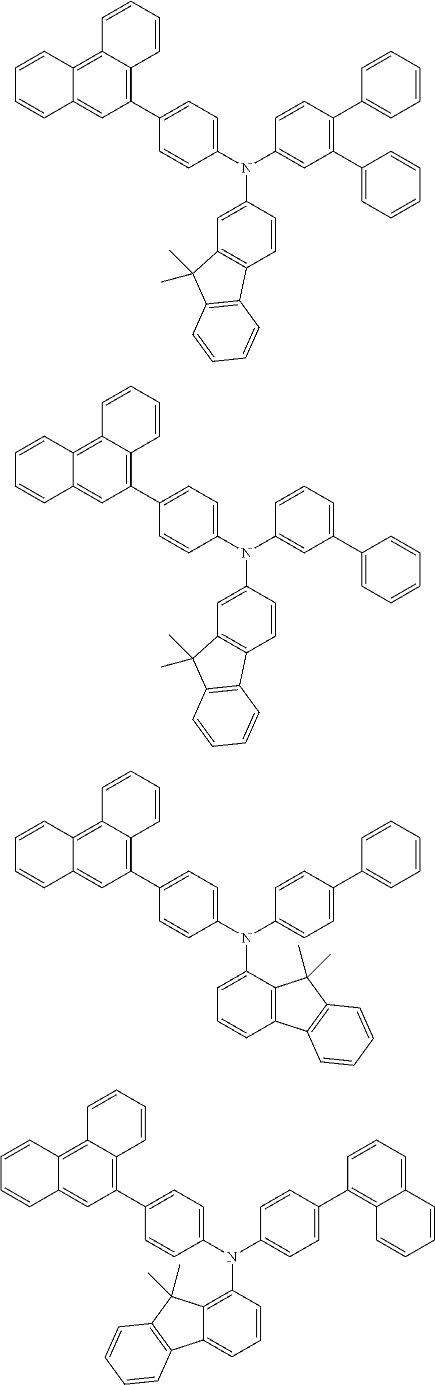 [SMILES: CC1(C)c2ccccc2-c2ccc(N(c3ccc(-c4cc5ccccc5c5ccccc45)cc3)c3ccc(-c4ccccc4)c(-c4ccccc4)c3)cc21 ; CC1(C)c2ccccc2-c2ccc(N(c3ccc(-c4cc5ccccc5c5ccccc45)cc3)c3cccc(-c4ccccc4)c3)cc21 ; CC1(C)c2ccccc2-c2cccc(N(c3ccc(-c4ccccc4)cc3)c3ccc(-c4cc5ccccc5c5ccccc45)cc3)c21 ; CC1(C)c2ccccc2-c2cccc(N(c3ccc(-c4cccc5ccccc45)cc3)c3ccc(-c4cc5ccccc5c5ccccc45)cc3)c21]

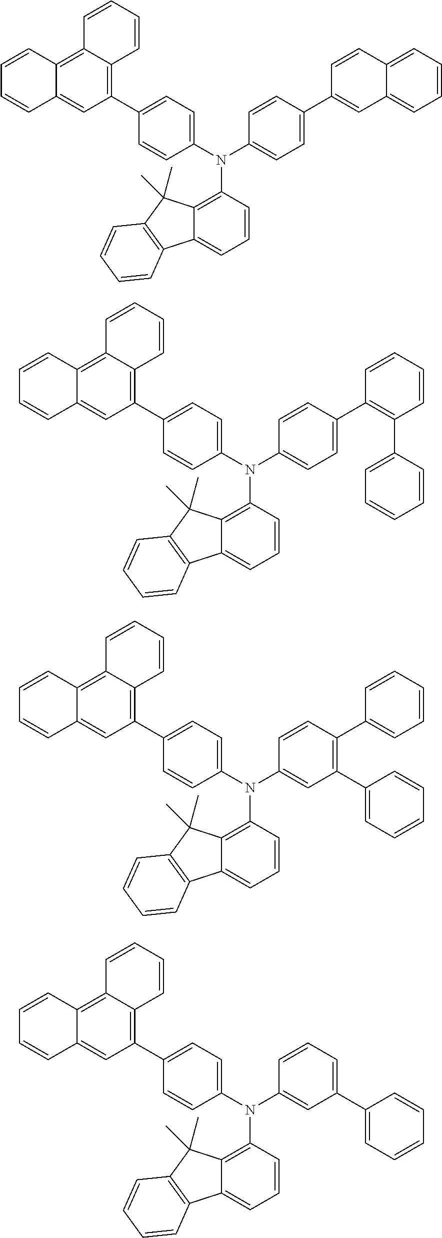

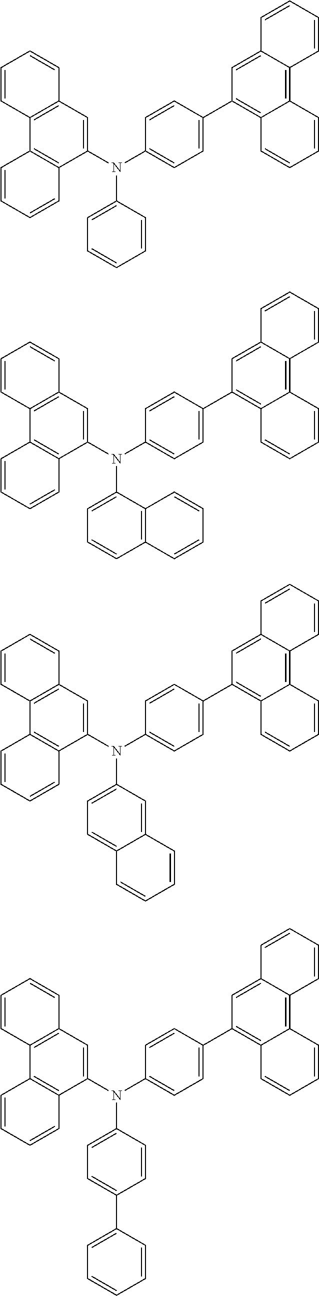 [SMILES: c1ccc(N(c2ccc(-c3cc4ccccc4c4ccccc34)cc2)c2cc3ccccc3c3ccccc23)cc1 ; c1ccc2c(N(c3ccc(-c4cc5ccccc5c5ccccc45)cc3)c3cc4ccccc4c4ccccc34)cccc2c1 ; c1ccc2cc(N(c3ccc(-c4cc5ccccc5c5ccccc45)cc3)c3cc4ccccc4c4ccccc34)ccc2c1 ; c1ccc(-c2ccc(N(c3ccc(-c4cc5ccccc5c5ccccc45)cc3)c3cc4ccccc4c4ccccc34)cc2)cc1]

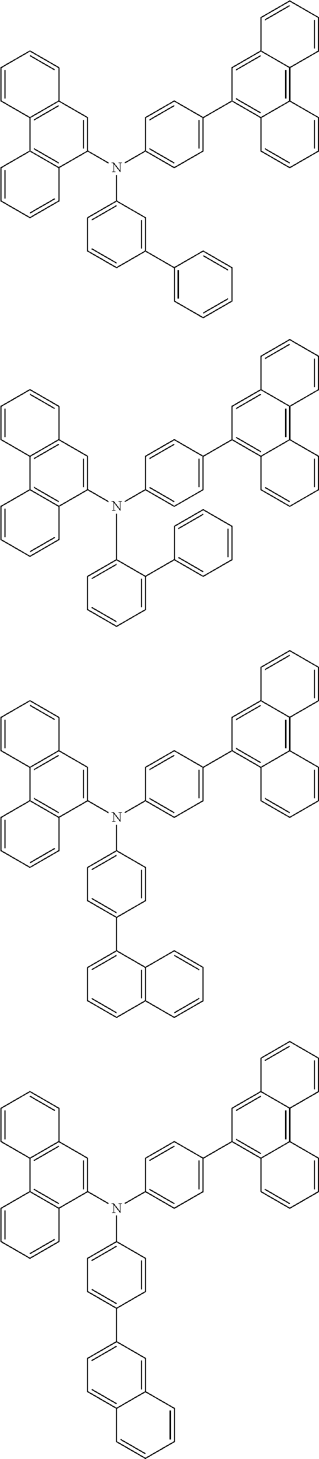 [SMILES: c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1 ; c1ccc2c(-c3ccc(N(c4ccc(-c5cc6ccccc6c6ccccc56)cc4)c4cc5ccccc5c5ccccc45)cc3)cccc2c1 ; c1ccc2cc(-c3ccc(N(c4ccc(-c5cc6ccccc6c6ccccc56)cc4)c4cc5ccccc5c5ccccc45)cc3)ccc2c1]

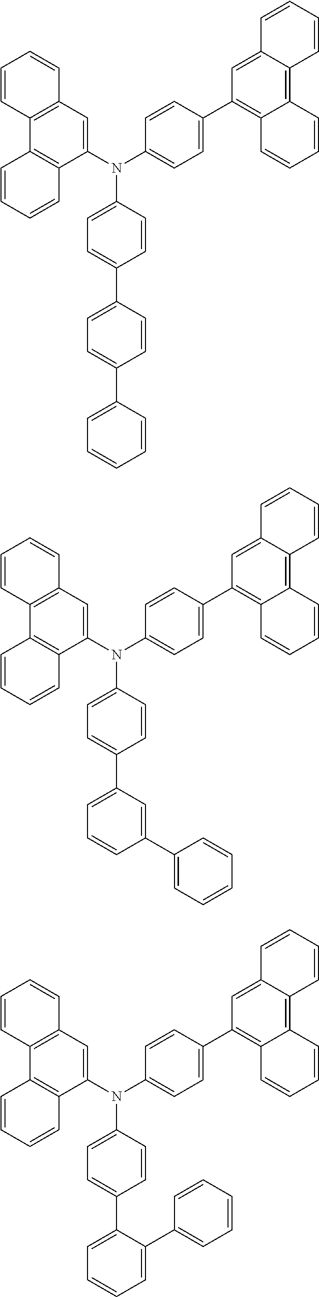 [SMILES: c1ccc(-c2ccc(-c3ccc(N(c4ccc(-c5cc6ccccc6c6ccccc56)cc4)c4cc5ccccc5c5ccccc45)cc3)cc2)cc1 ; c1ccc(-c2cccc(-c3ccc(N(c4ccc(-c5cc6ccccc6c6ccccc56)cc4)c4cc5ccccc5c5ccccc45)cc3)c2)cc1 ; c1ccc(-c2ccccc2-c2ccc(N(c3ccc(-c4cc5ccccc5c5ccccc45)cc3)c3cc4ccccc4c4ccccc34)cc2)cc1]

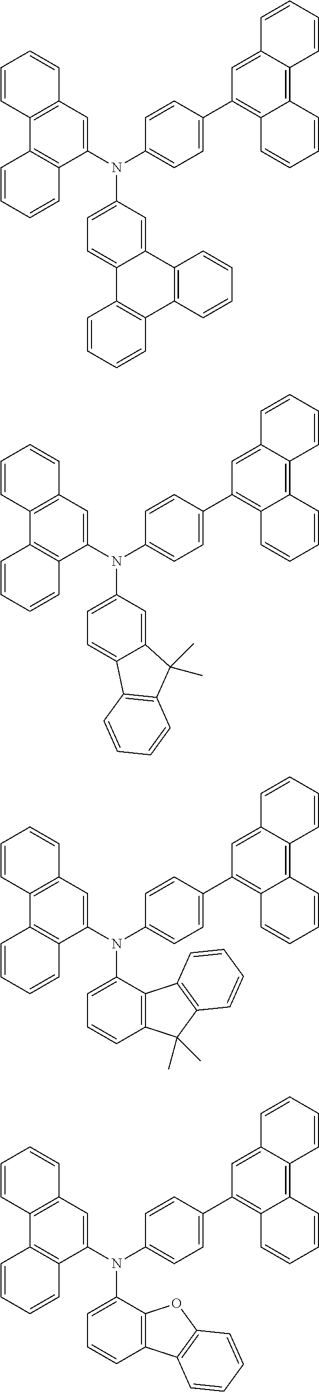 [SMILES: c1ccc2c(c1)cc(-c1ccc(N(c3ccc4c5ccccc5c5ccccc5c4c3)c3cc4ccccc4c4ccccc34)cc1)c1ccccc12 ; CC1(C)c2ccccc2-c2ccc(N(c3ccc(-c4cc5ccccc5c5ccccc45)cc3)c3cc4ccccc4c4ccccc34)cc21 ; CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12]

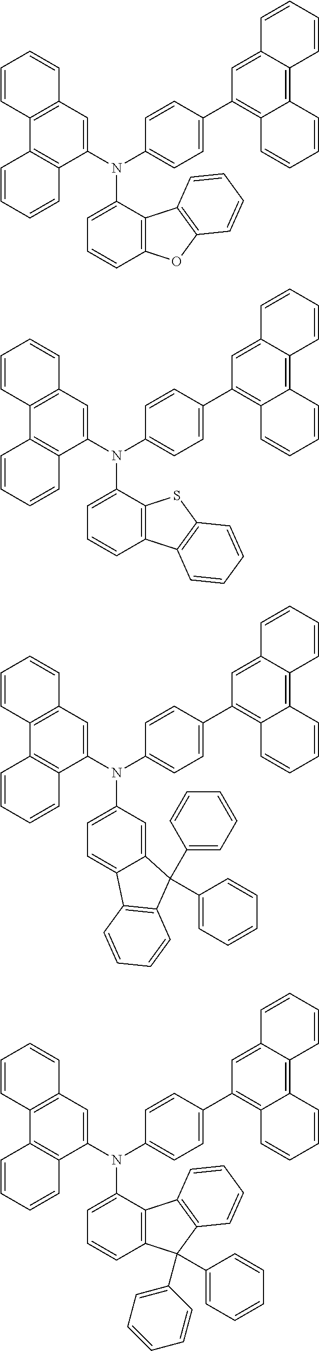 [SMILES: c1ccc2c(c1)cc(-c1ccc(N(c3cc4ccccc4c4ccccc34)c3cccc4oc5ccccc5c34)cc1)c1ccccc12 ; c1ccc2c(c1)cc(-c1ccc(N(c3cc4ccccc4c4ccccc34)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4cc5ccccc5c5ccccc45)cc32)cc1 ; c1ccc(C2(c3ccccc3)c3ccccc3-c3c(N(c4ccc(-c5cc6ccccc6c6ccccc56)cc4)c4cc5ccccc5c5ccccc45)cccc32)cc1]

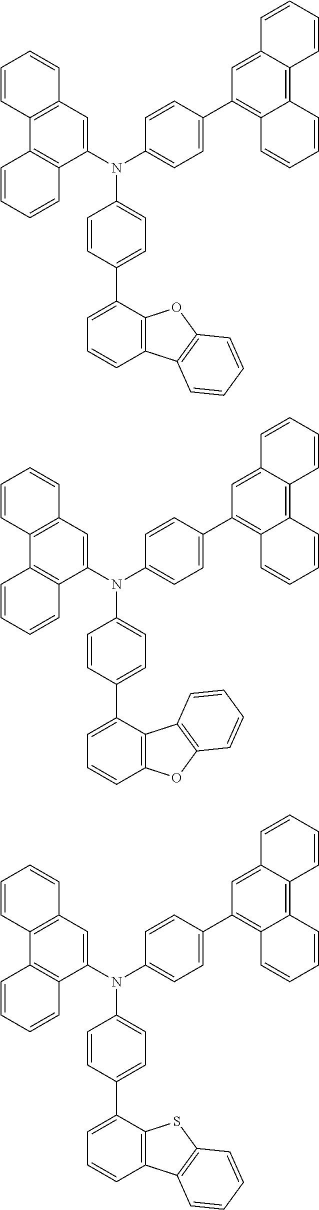 [SMILES: c1ccc2c(c1)cc(-c1ccc(N(c3ccc(-c4cccc5c4oc4ccccc45)cc3)c3cc4ccccc4c4ccccc34)cc1)c1ccccc12 ; c1ccc2c(c1)cc(-c1ccc(N(c3ccc(-c4cccc5oc6ccccc6c45)cc3)c3cc4ccccc4c4ccccc34)cc1)c1ccccc12 ; c1ccc2c(c1)cc(-c1ccc(N(c3ccc(-c4cccc5c4sc4ccccc45)cc3)c3cc4ccccc4c4ccccc34)cc1)c1ccccc12]

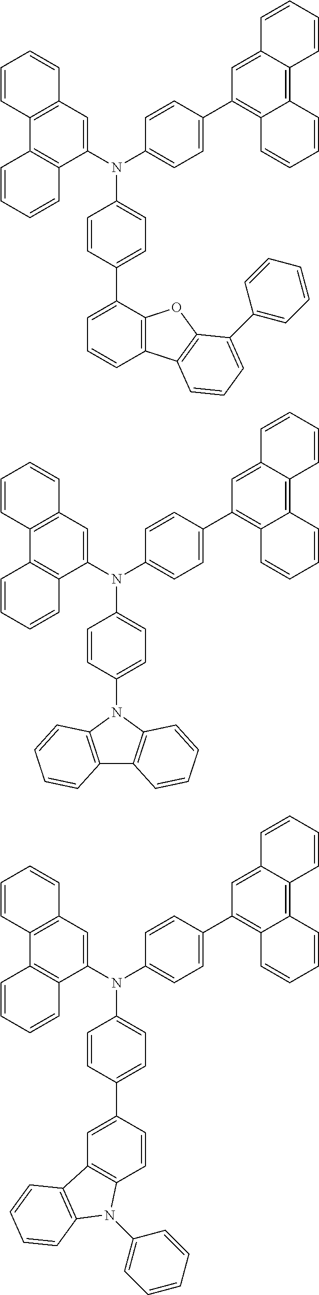 [SMILES: c1ccc(-c2cccc3c2oc2c(-c4ccc(N(c5ccc(-c6cc7ccccc7c7ccccc67)cc5)c5cc6ccccc6c6ccccc56)cc4)cccc23)cc1 ; c1ccc2c(c1)cc(-c1ccc(N(c3ccc(-n4c5ccccc5c5ccccc54)cc3)c3cc4ccccc4c4ccccc34)cc1)c1ccccc12 ; c1ccc(-n2c3ccccc3c3cc(-c4ccc(N(c5ccc(-c6cc7ccccc7c7ccccc67)cc5)c5cc6ccccc6c6ccccc56)cc4)ccc32)cc1]

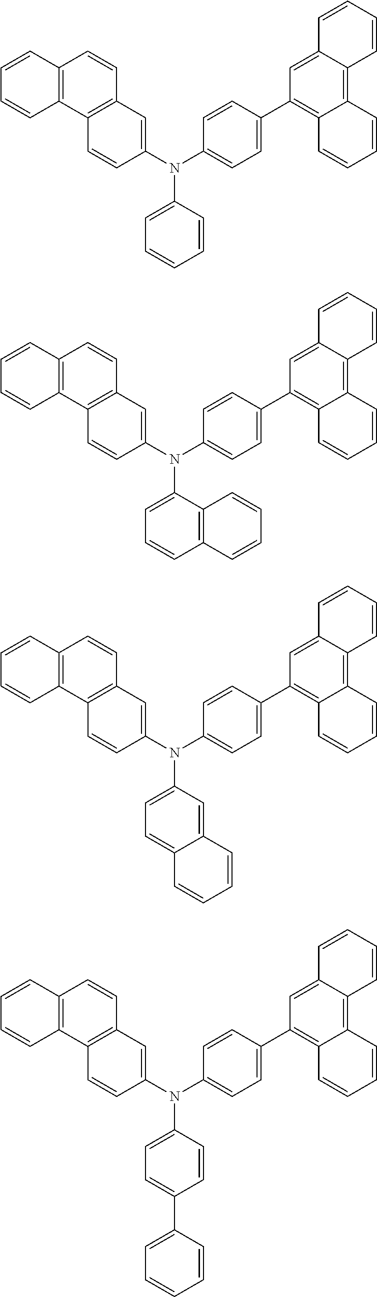 [SMILES: c1ccc(N(c2ccc(-c3cc4ccccc4c4ccccc34)cc2)c2ccc3c(ccc4ccccc43)c2)cc1 ; c1ccc2c(N(c3ccc(-c4cc5ccccc5c5ccccc45)cc3)c3ccc4c(ccc5ccccc54)c3)cccc2c1 ; c1ccc2cc(N(c3ccc(-c4cc5ccccc5c5ccccc45)cc3)c3ccc4c(ccc5ccccc54)c3)ccc2c1 ; c1ccc(-c2ccc(N(c3ccc(-c4cc5ccccc5c5ccccc45)cc3)c3ccc4c(ccc5ccccc54)c3)cc2)cc1]

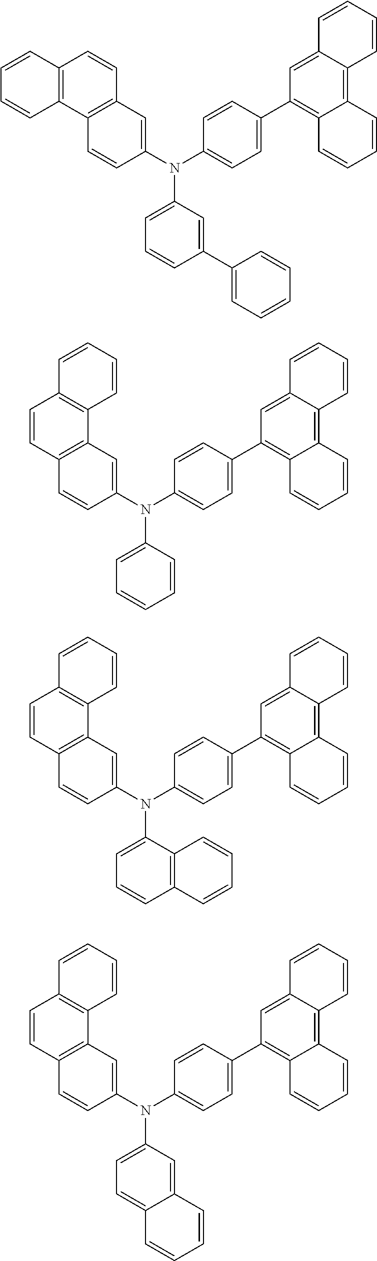 [SMILES: c1ccc(-c2cccc(N(c3ccc(-c4cc5ccccc5c5ccccc45)cc3)c3ccc4c(ccc5ccccc54)c3)c2)cc1 ; c1ccc(N(c2ccc(-c3cc4ccccc4c4ccccc34)cc2)c2ccc3ccc4ccccc4c3c2)cc1 ; c1ccc2c(N(c3ccc(-c4cc5ccccc5c5ccccc45)cc3)c3ccc4ccc5ccccc5c4c3)cccc2c1 ; c1ccc2cc(N(c3ccc(-c4cc5ccccc5c5ccccc45)cc3)c3ccc4ccc5ccccc5c4c3)ccc2c1]

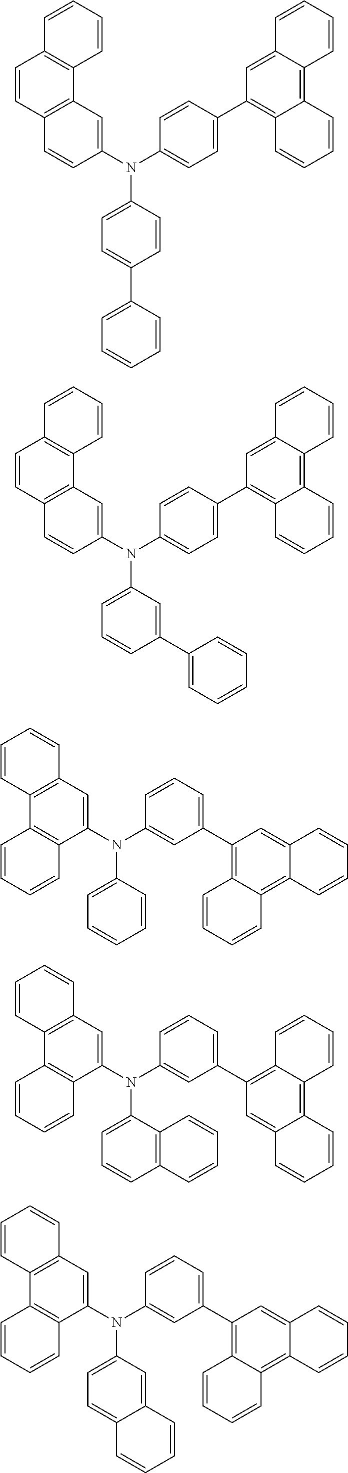

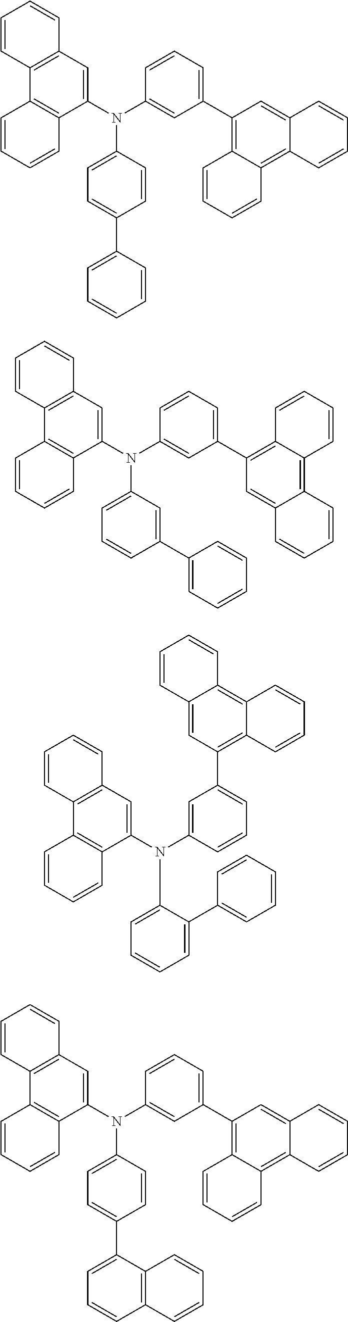

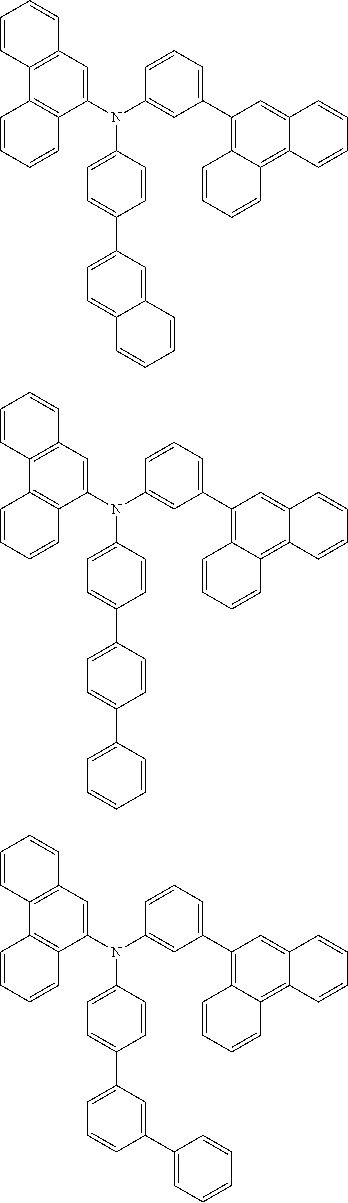 [SMILES: c1cc(-c2cc3ccccc3c3ccccc23)cc(N(c2ccc(-c3ccc4ccccc4c3)cc2)c2cc3ccccc3c3ccccc23)c1 ; c1ccc(-c2ccc(-c3ccc(N(c4cccc(-c5cc6ccccc6c6ccccc56)c4)c4cc5ccccc5c5ccccc45)cc3)cc2)cc1 ; c1ccc(-c2cccc(-c3ccc(N(c4cccc(-c5cc6ccccc6c6ccccc56)c4)c4cc5ccccc5c5ccccc45)cc3)c2)cc1]

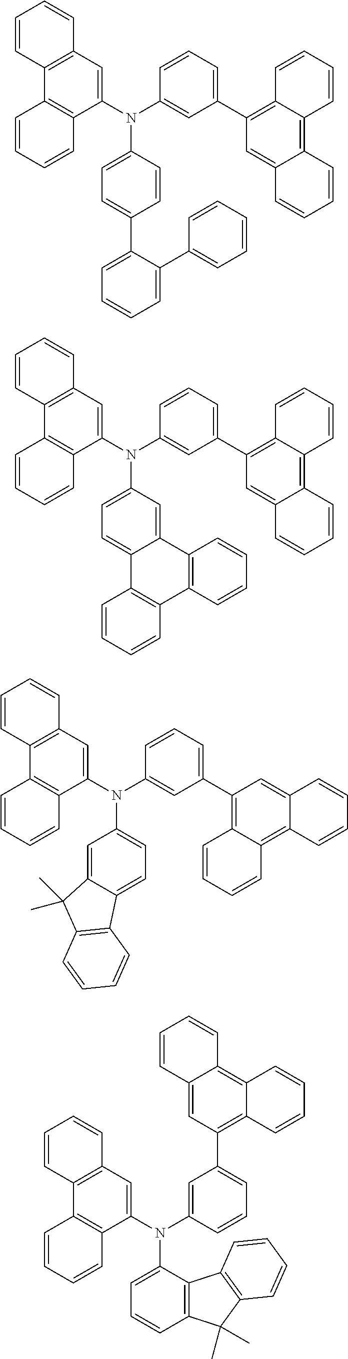 [SMILES: c1ccc(-c2ccccc2-c2ccc(N(c3cccc(-c4cc5ccccc5c5ccccc45)c3)c3cc4ccccc4c4ccccc34)cc2)cc1 ; c1cc(-c2cc3ccccc3c3ccccc23)cc(N(c2ccc3c4ccccc4c4ccccc4c3c2)c2cc3ccccc3c3ccccc23)c1 ; CC1(C)c2ccccc2-c2ccc(N(c3cccc(-c4cc5ccccc5c5ccccc45)c3)c3cc4ccccc4c4ccccc34)cc21 ; CC1(C)c2ccccc2-c2c(N(c3cccc(-c4cc5ccccc5c5ccccc45)c3)c3cc4ccccc4c4ccccc34)cccc21]

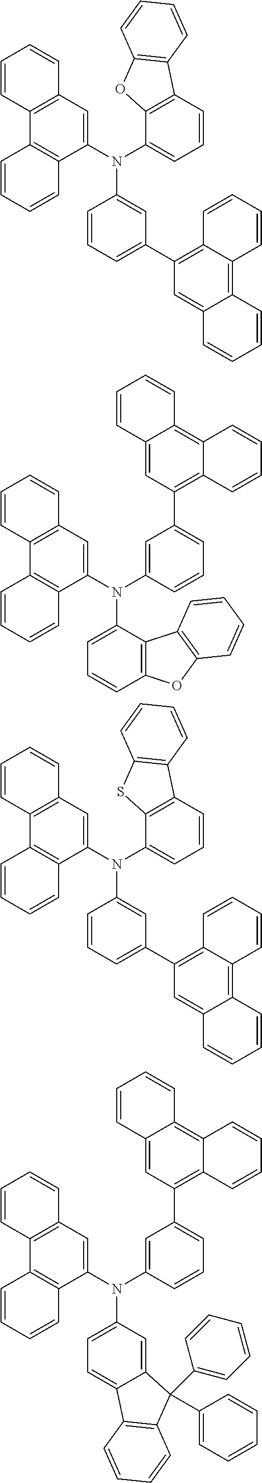

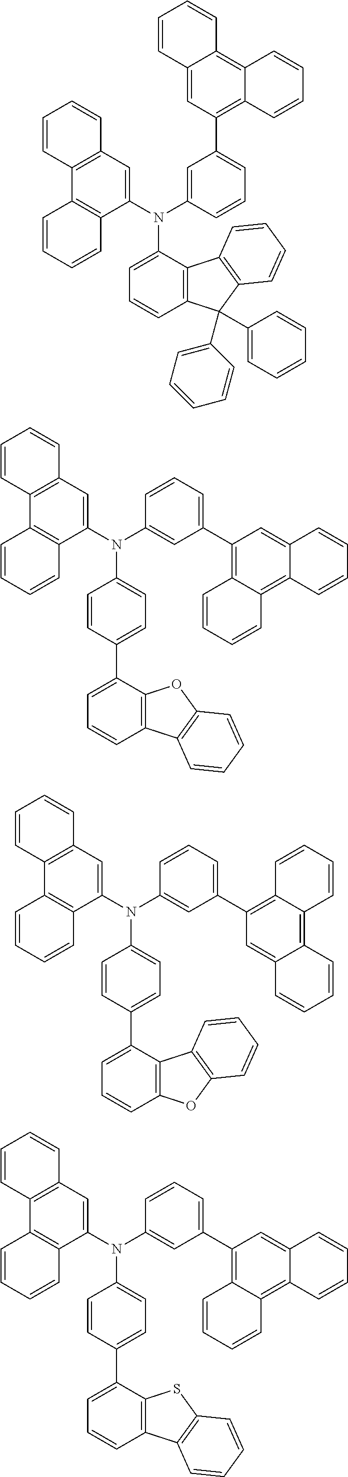 [SMILES: c1ccc(C2(c3ccccc3)c3ccccc3-c3c(N(c4cccc(-c5cc6ccccc6c6ccccc56)c4)c4cc5ccccc5c5ccccc45)cccc32)cc1 ; c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1 ; c1cc(-c2cc3ccccc3c3ccccc23)cc(N(c2ccc(-c3cccc4c3sc3ccccc34)cc2)c2cc3ccccc3c3ccccc23)c1]

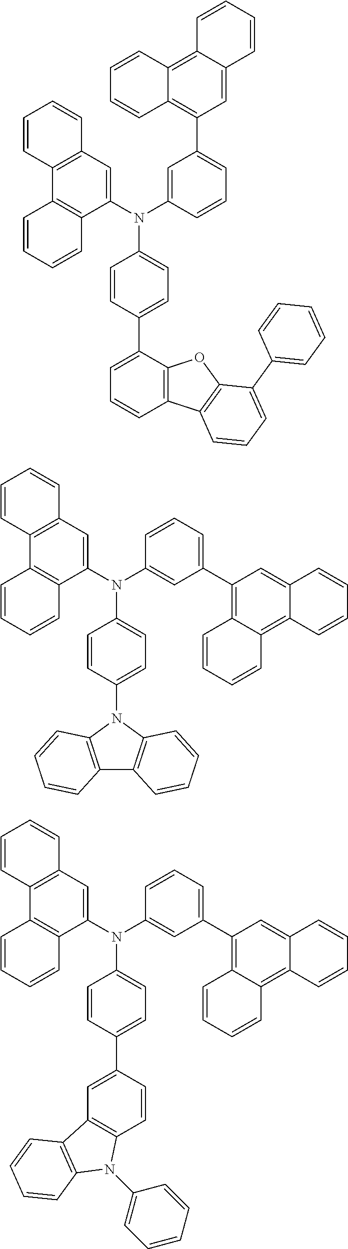

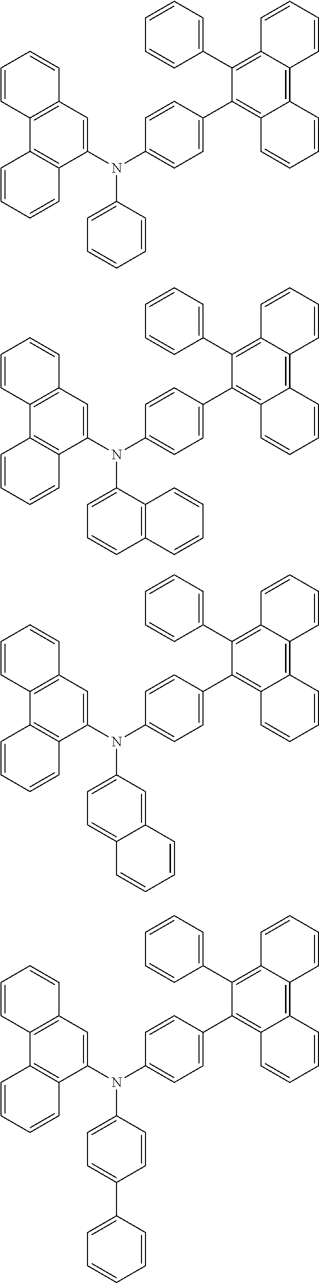

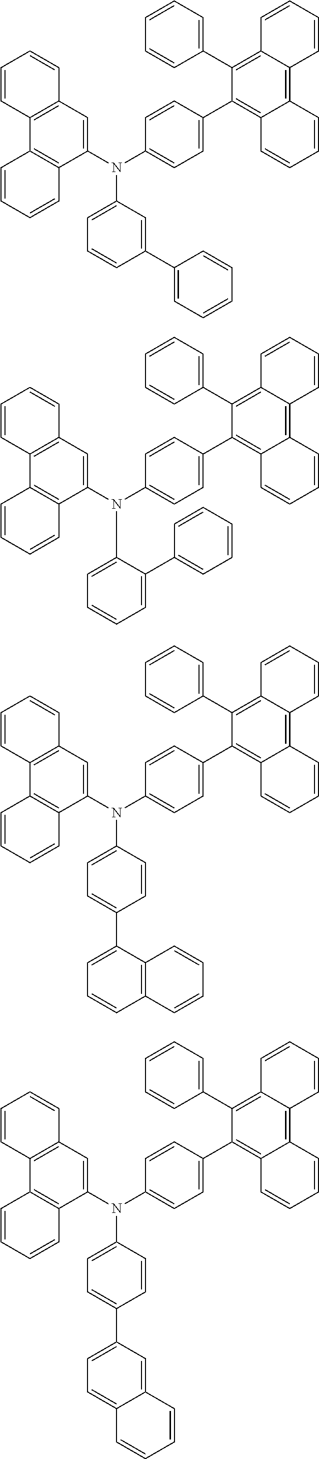

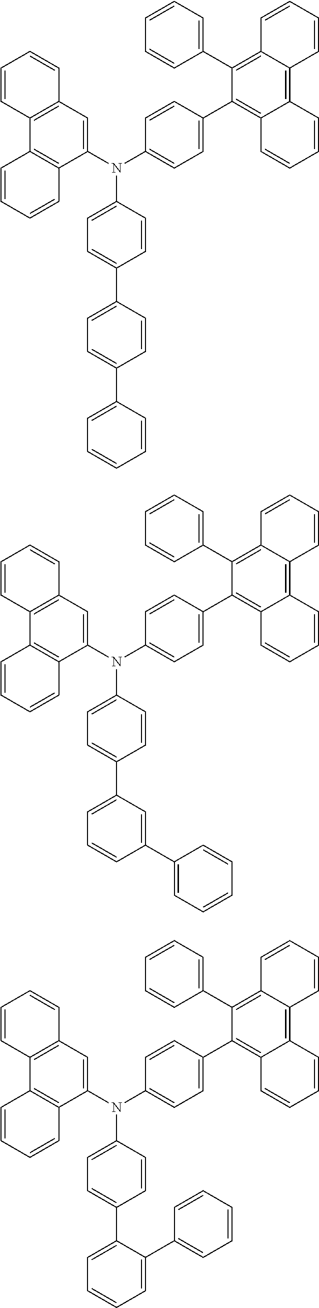

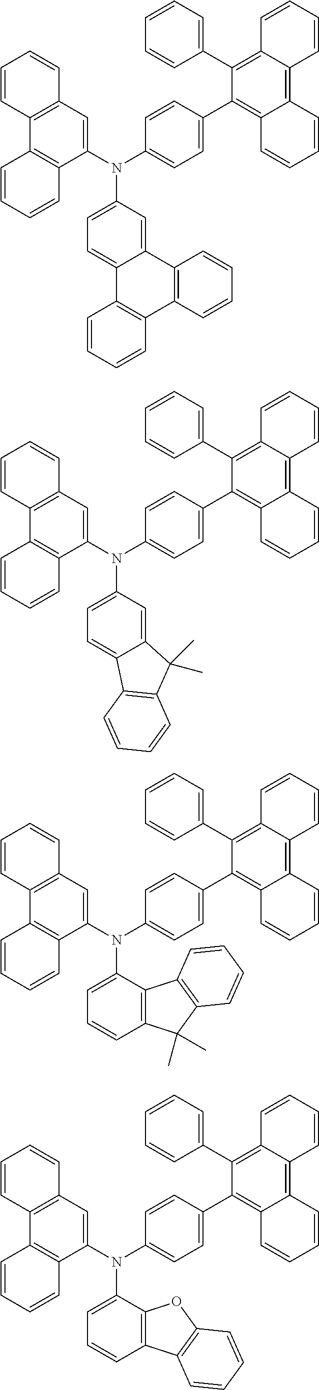

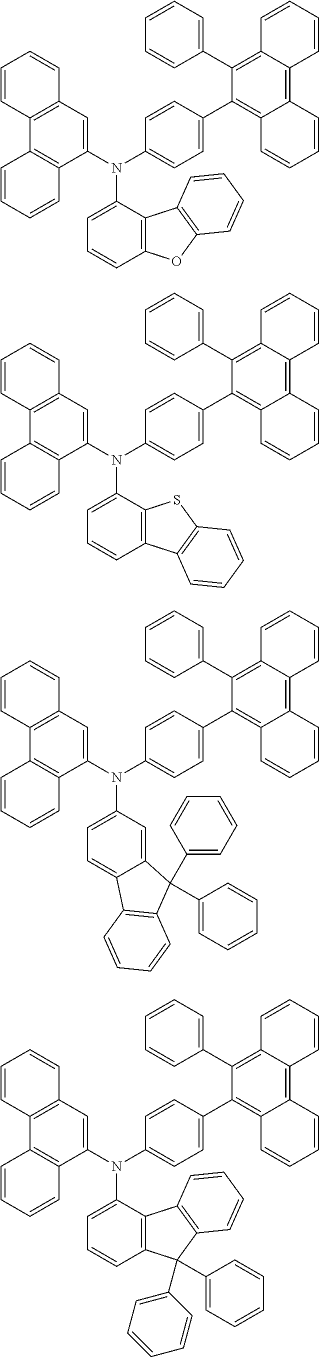

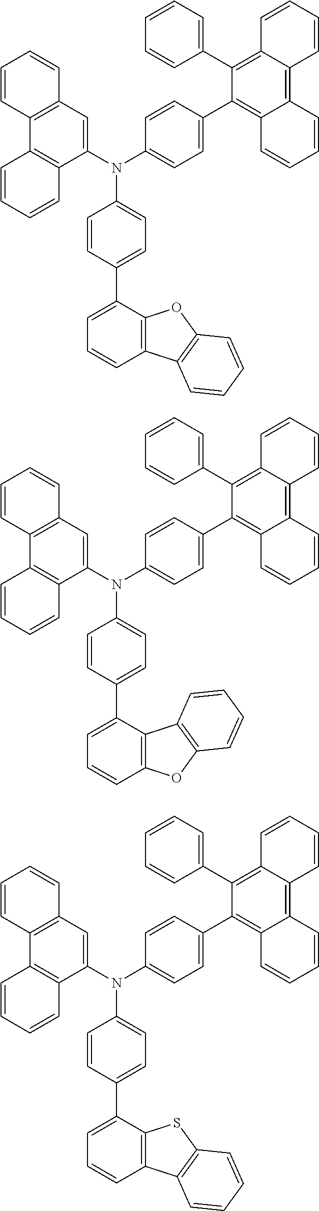

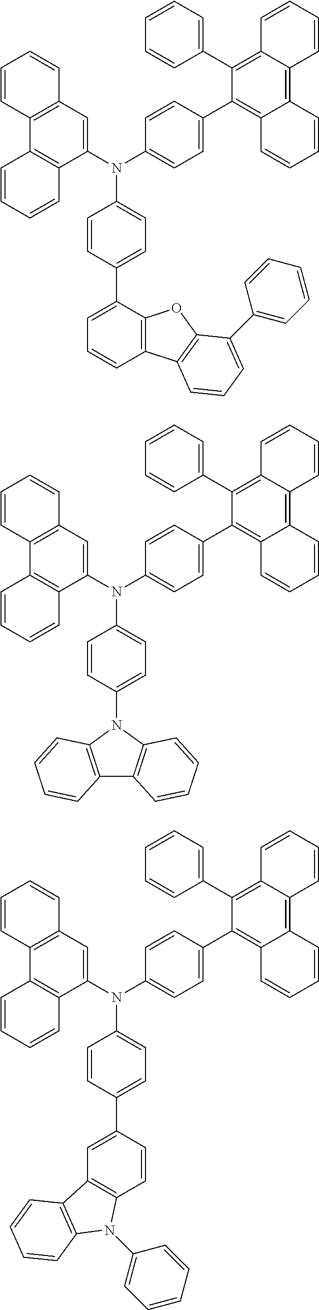

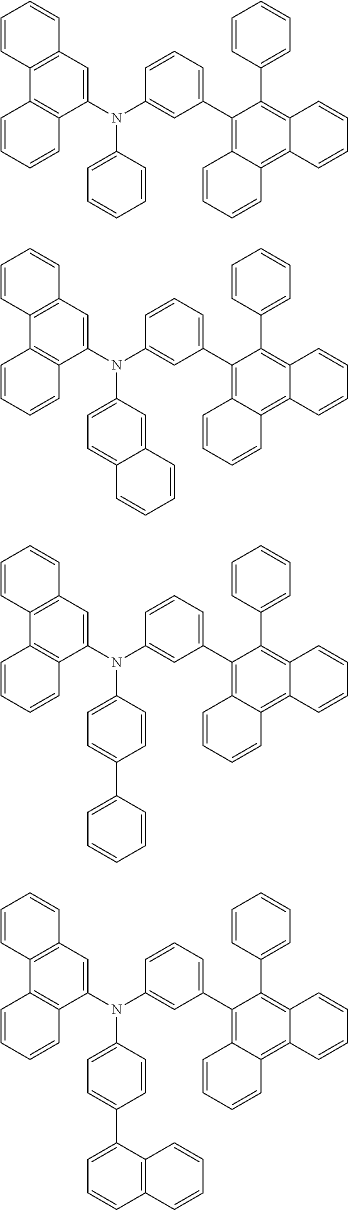

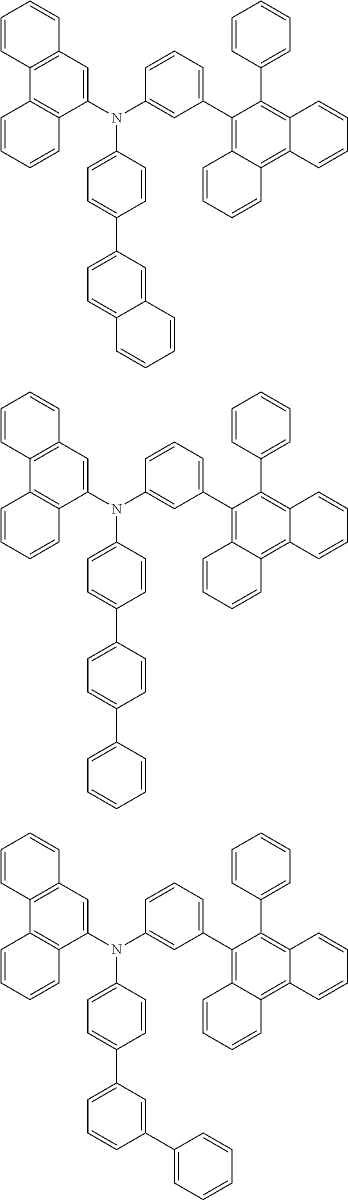 [SMILES: c1ccc(-c2c(-c3cccc(N(c4ccc(-c5ccc6ccccc6c5)cc4)c4cc5ccccc5c5ccccc45)c3)c3ccccc3c3ccccc23)cc1 ; c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1]

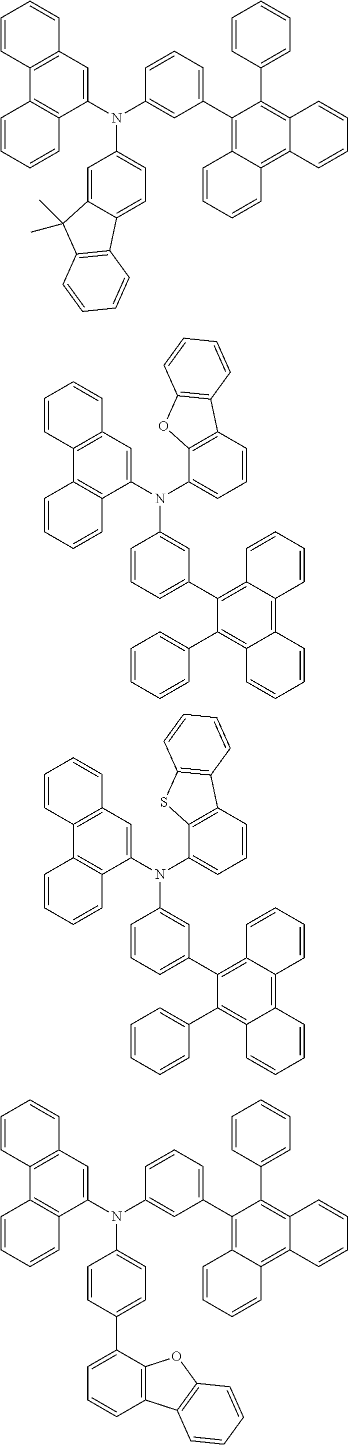

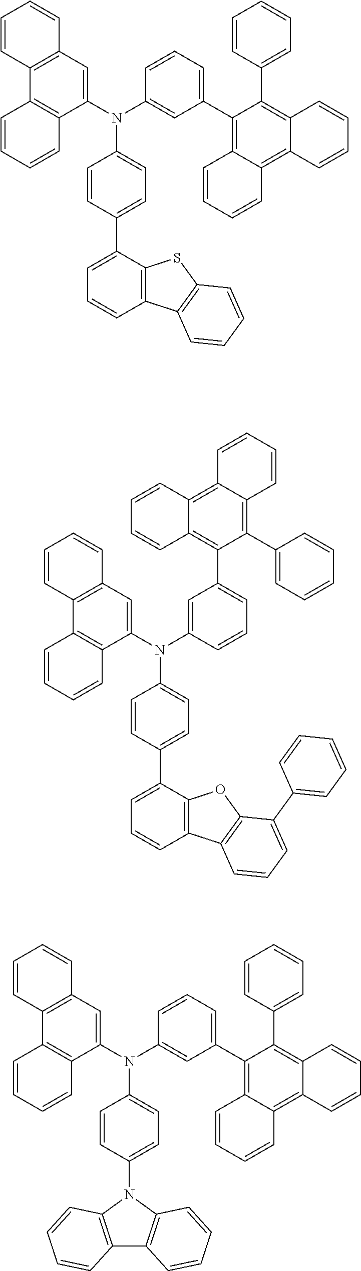 [SMILES: c1ccc(-c2c(-c3cccc(N(c4ccc(-c5cccc6c5sc5ccccc56)cc4)c4cc5ccccc5c5ccccc45)c3)c3ccccc3c3ccccc23)cc1 ; c1ccc(-c2c(-c3cccc(N(c4ccc(-c5cccc6c5oc5c(-c7ccccc7)cccc56)cc4)c4cc5ccccc5c5ccccc45)c3)c3ccccc3c3ccccc23)cc1 ; c1ccc(-c2c(-c3cccc(N(c4ccc(-n5c6ccccc6c6ccccc65)cc4)c4cc5ccccc5c5ccccc45)c3)c3ccccc3c3ccccc23)cc1]

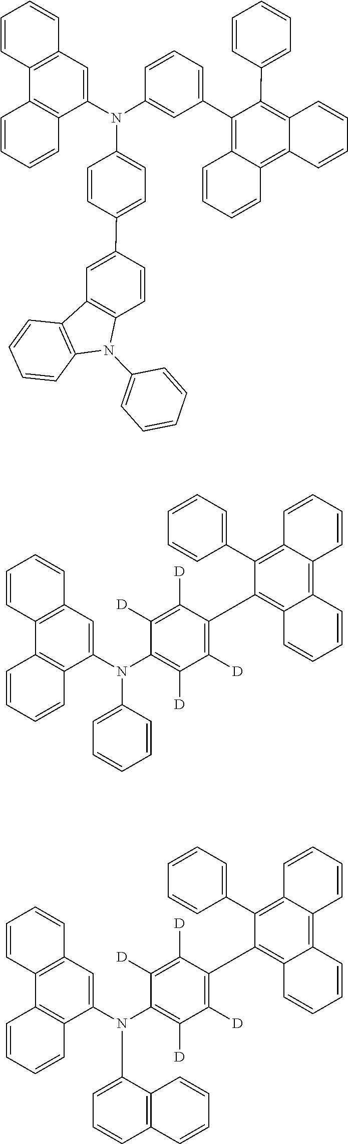 [SMILES: c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1 ; [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12]

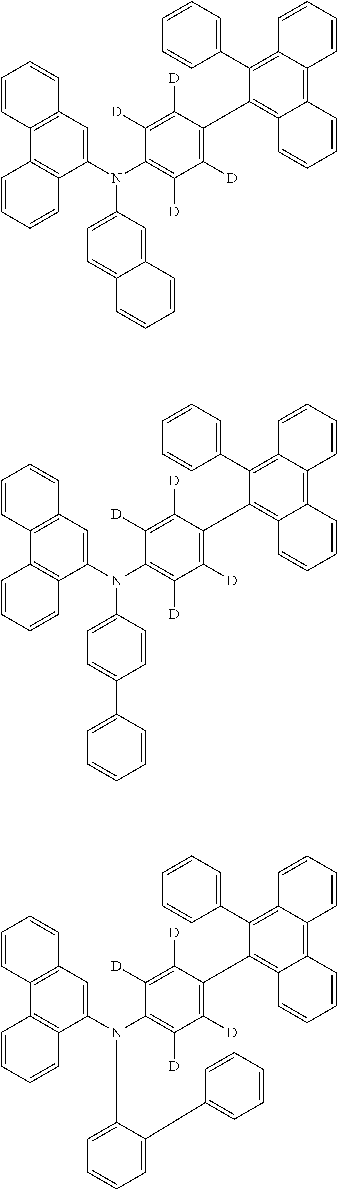 [SMILES: [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12]

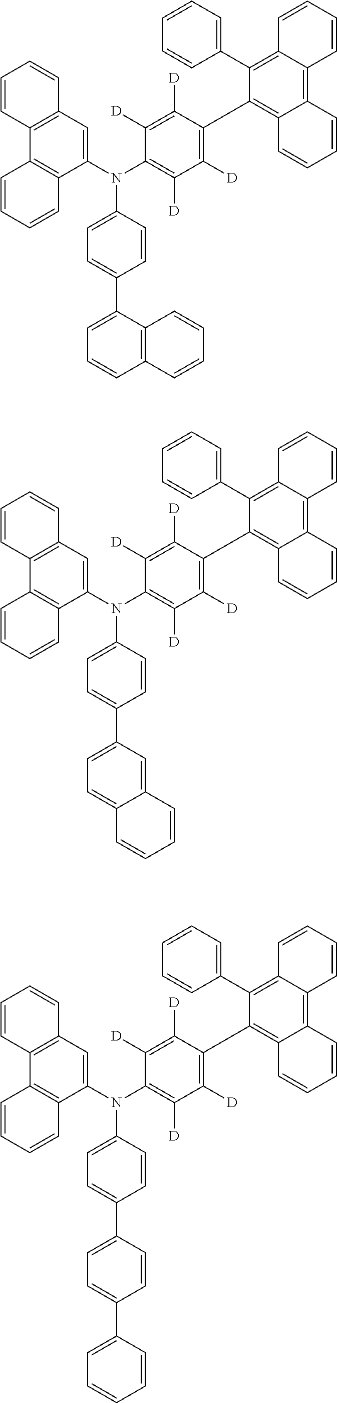 [SMILES: [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12]

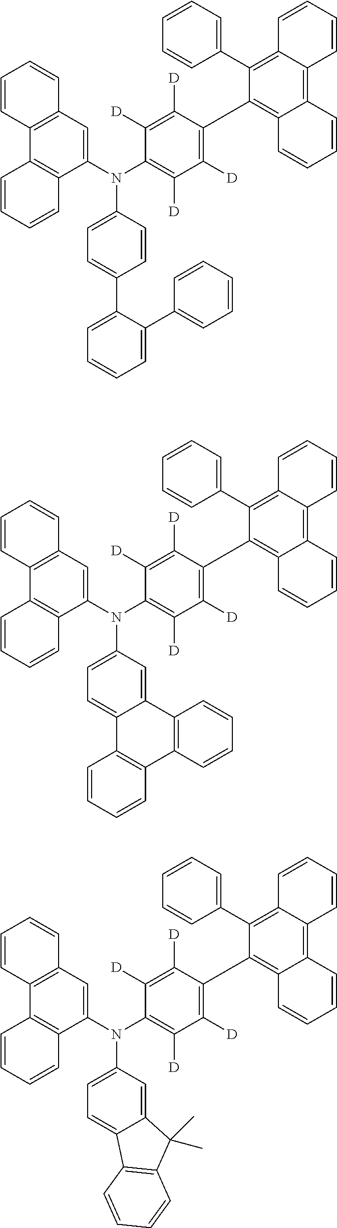 [SMILES: [2H]c1c([2H])c(N(c2ccc(-c3ccccc3-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccc3c4ccccc4c4ccccc4c3c2)c2cc3ccccc3c3ccccc23)c([2H])c([2H])c1-c1c(-c2ccccc2)c2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1c(-c2ccccc2)c2ccccc2c2ccccc12]

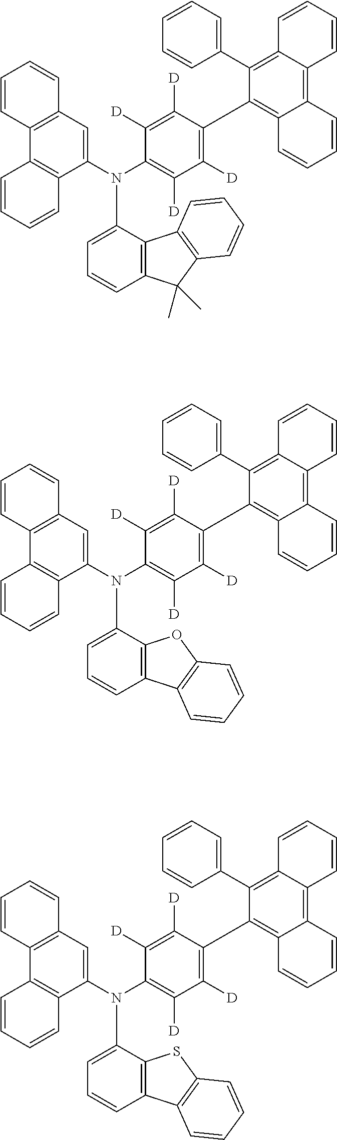 [SMILES: [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2oc2ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2sc2ccccc23)c([2H])c([2H])c1-c1c(-c2ccccc2)c2ccccc2c2ccccc12]

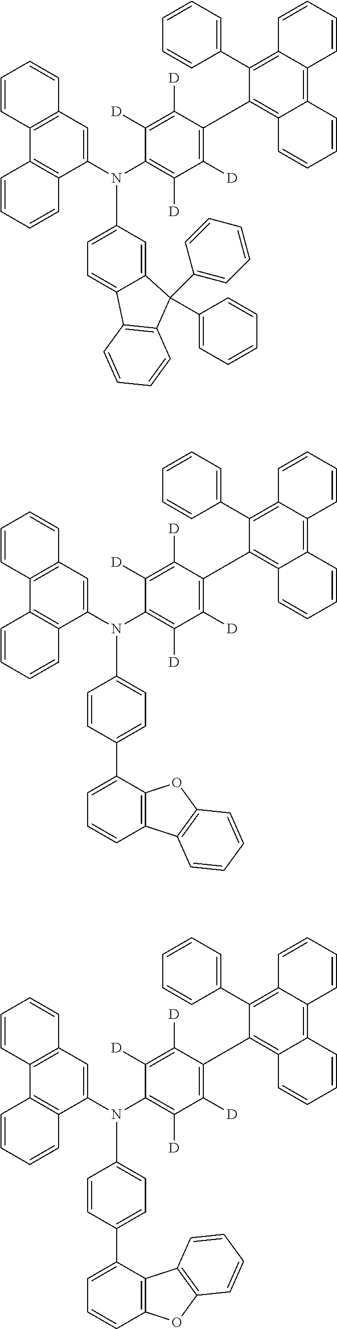 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12]

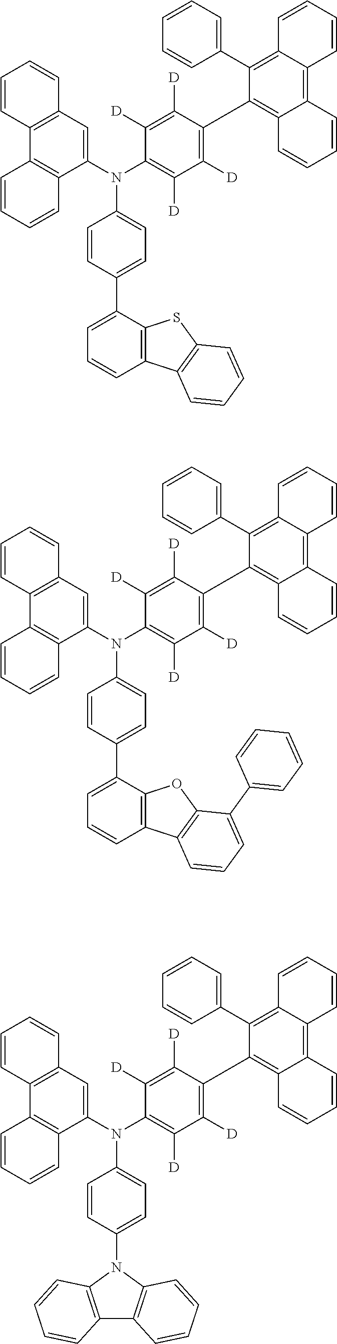 [SMILES: [2H]c1c([2H])c(N(c2ccc(-c3cccc4c3sc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1c(-c2ccccc2)c2ccccc2c2ccccc12]

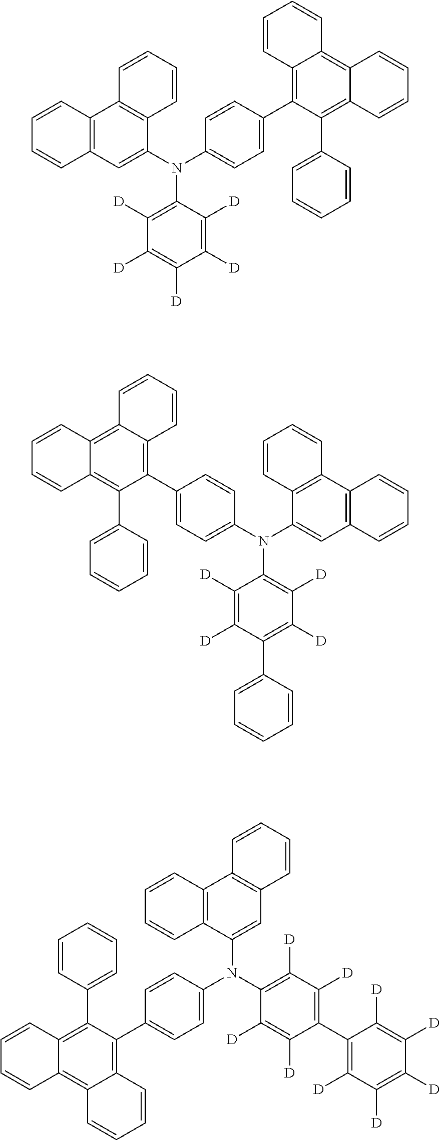 [SMILES: [2H]c1c([2H])c([2H])c(N(c2ccc(-c3c(-c4ccccc4)c4ccccc4c4ccccc34)cc2)c2cc3ccccc3c3ccccc23)c([2H])c1[2H] ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4c(-c5ccccc5)c5ccccc5c5ccccc45)cc3)c3cc4ccccc4c4ccccc34)c([2H])c2[2H])c([2H])c1[2H]]

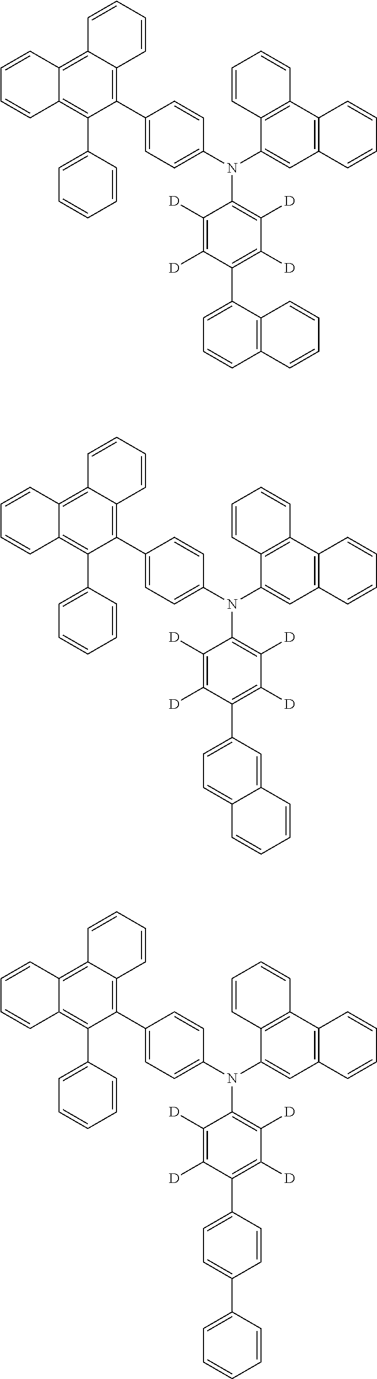 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1]

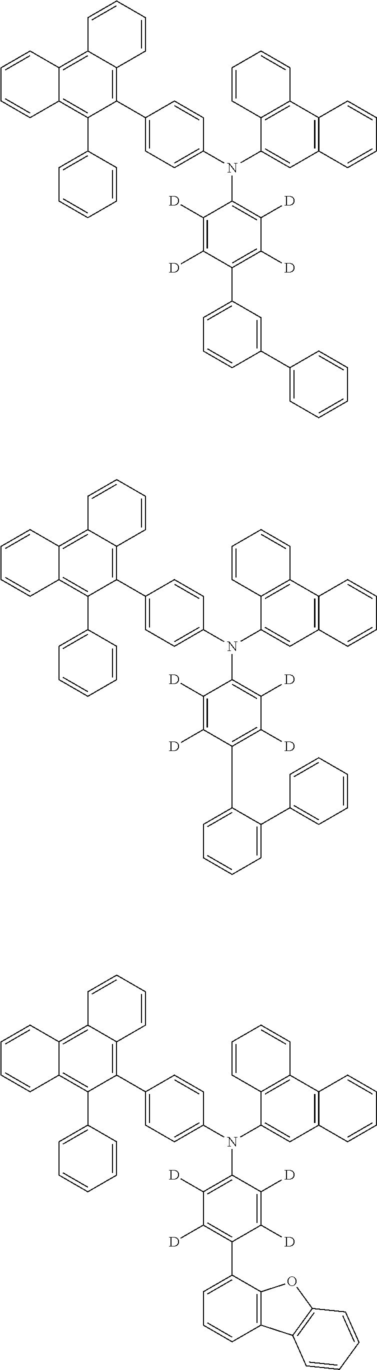 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12]

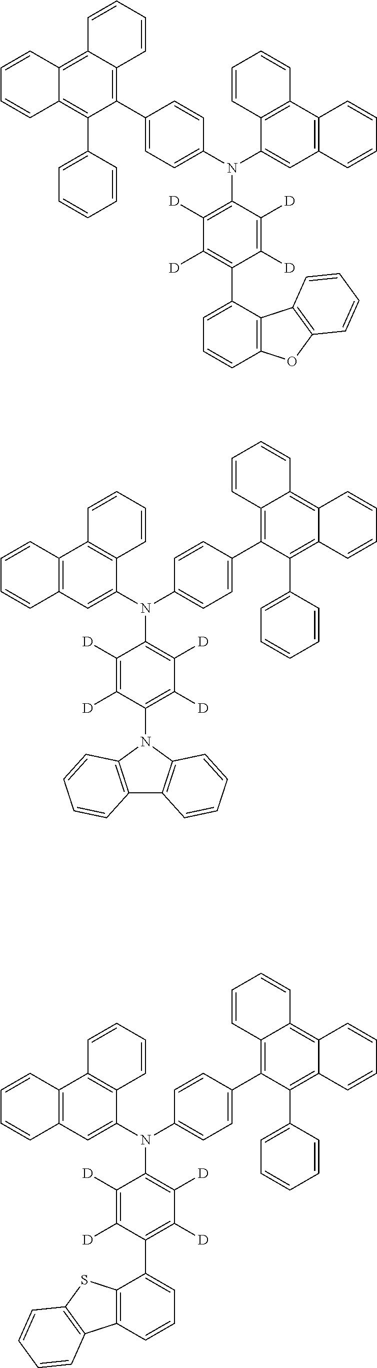 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12 ; [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12]

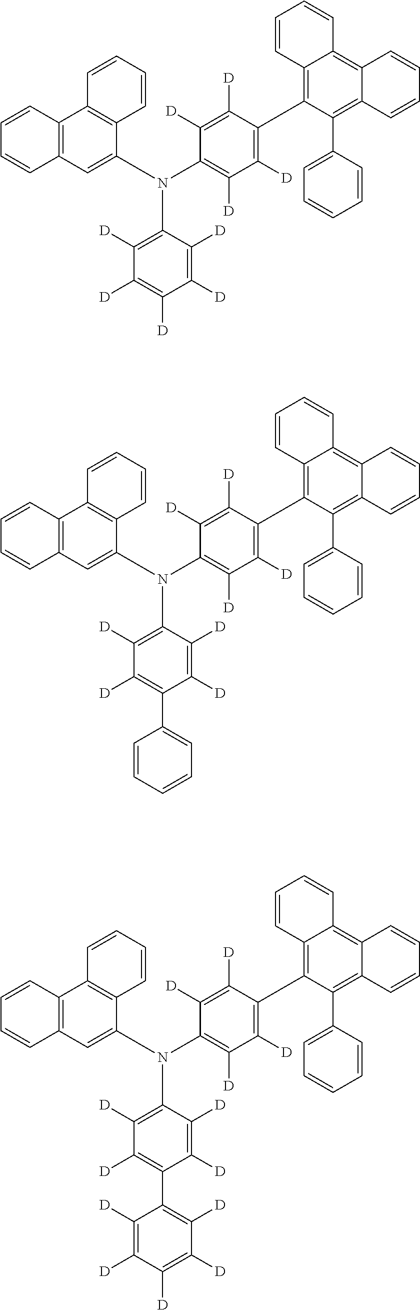 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H]]

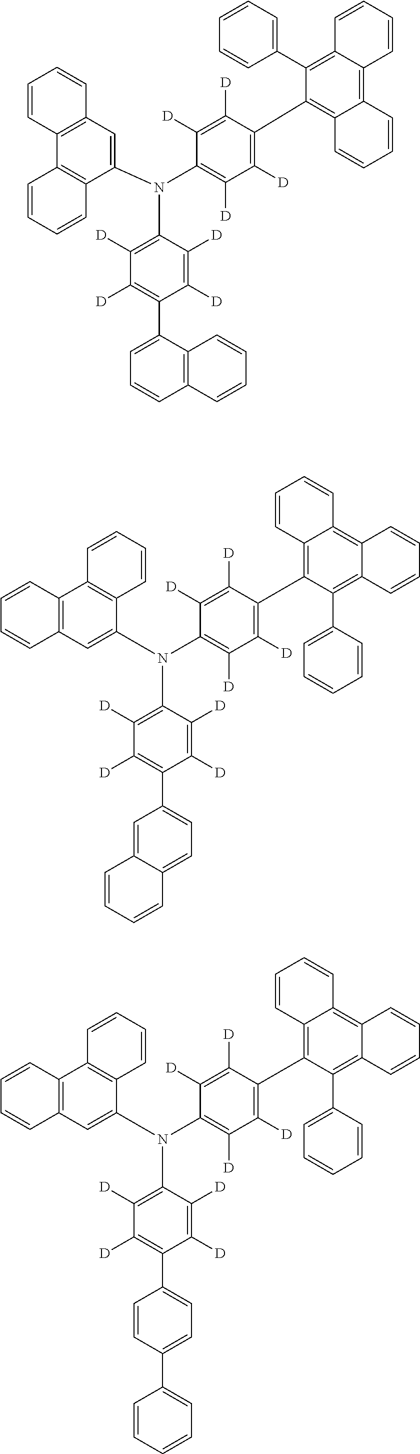 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1]

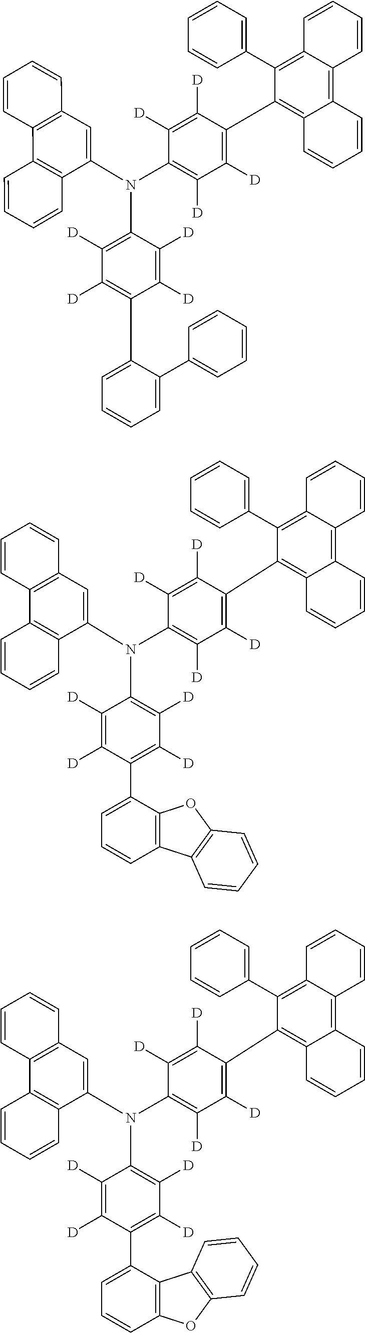

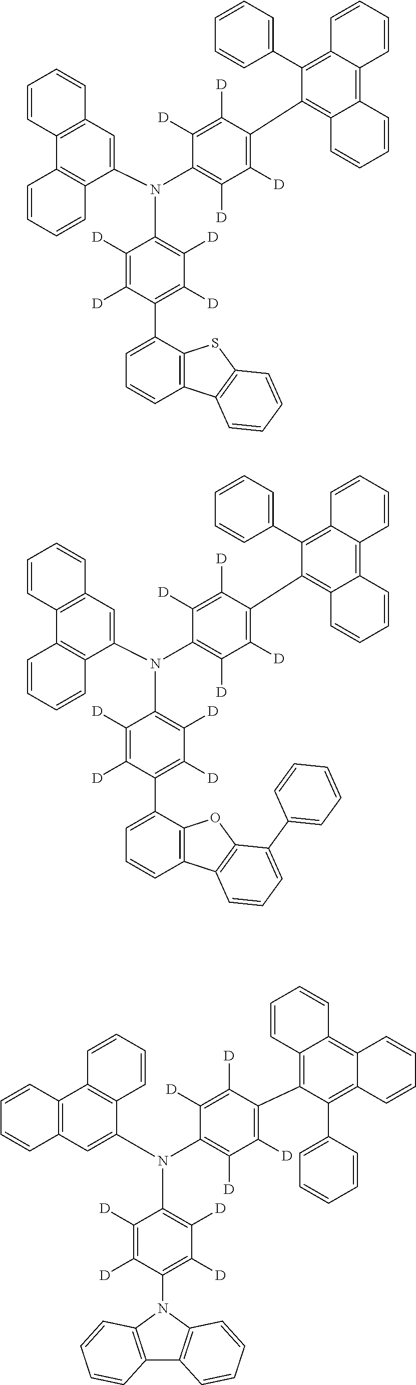 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1c(-c2ccccc2)c2ccccc2c2ccccc12]

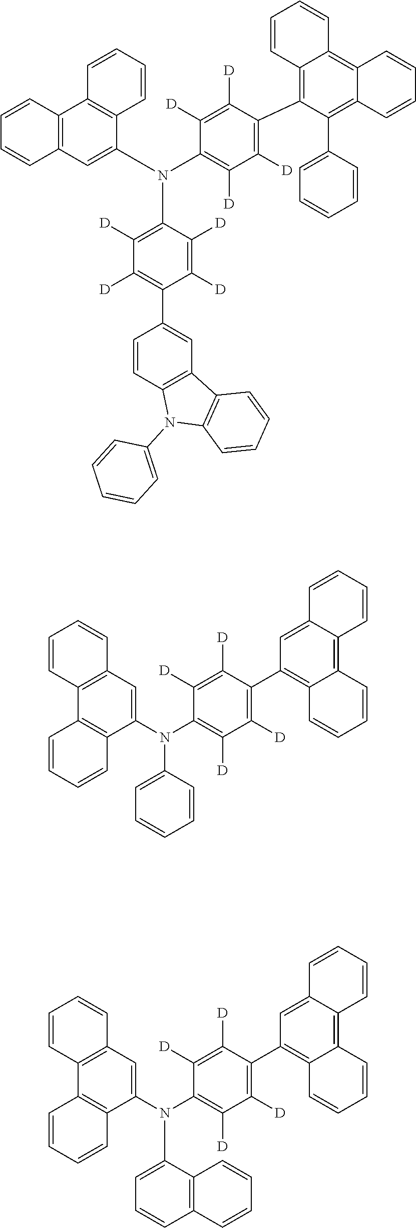 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2c(c1)c1ccccc1n2-c1ccccc1 ; [2H]c1c([2H])c(N(c2ccccc2)c2cc3ccccc3c3ccccc23)c([2H])c([2H])c1-c1cc2ccccc2c2ccccc12 ; [2H]c1c([2H])c(N(c2cccc3ccccc23)c2cc3ccccc3c3ccccc23)c([2H])c([2H])c1-c1cc2ccccc2c2ccccc12]

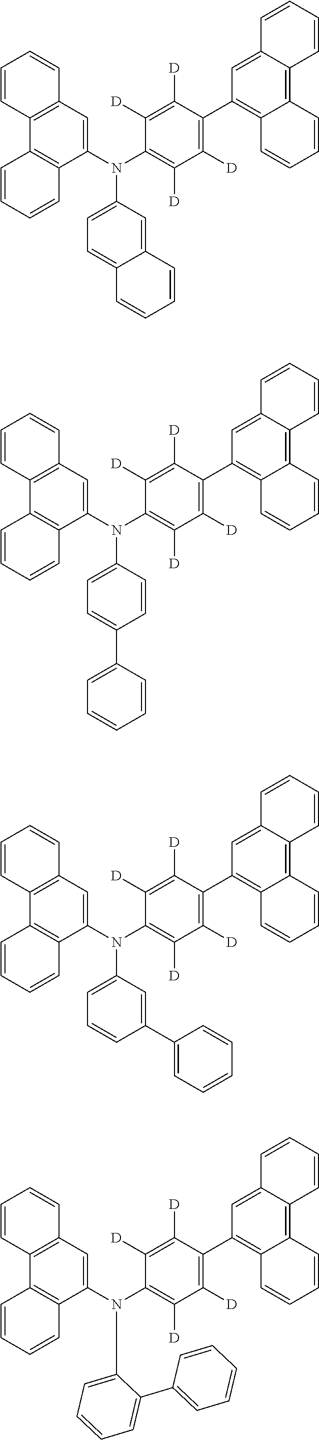 [SMILES: [2H]c1c([2H])c(N(c2ccc3ccccc3c2)c2cc3ccccc3c3ccccc23)c([2H])c([2H])c1-c1cc2ccccc2c2ccccc12 ; [2H]c1c([2H])c(N(c2ccc(-c3ccccc3)cc2)c2cc3ccccc3c3ccccc23)c([2H])c([2H])c1-c1cc2ccccc2c2ccccc12 ; [2H]c1c([2H])c(N(c2cccc(-c3ccccc3)c2)c2cc3ccccc3c3ccccc23)c([2H])c([2H])c1-c1cc2ccccc2c2ccccc12 ; [2H]c1c([2H])c(N(c2ccccc2-c2ccccc2)c2cc3ccccc3c3ccccc23)c([2H])c([2H])c1-c1cc2ccccc2c2ccccc12]

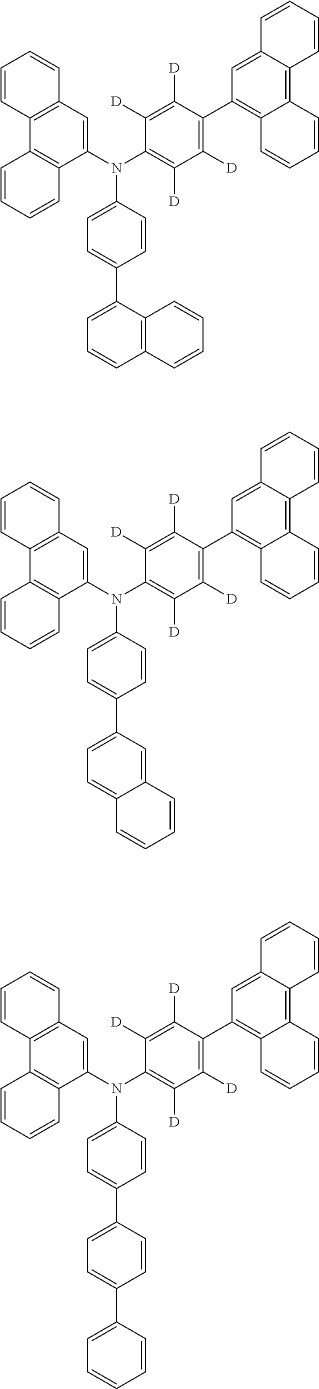 [SMILES: [2H]c1c([2H])c(N(c2ccc(-c3cccc4ccccc34)cc2)c2cc3ccccc3c3ccccc23)c([2H])c([2H])c1-c1cc2ccccc2c2ccccc12 ; [2H]c1c([2H])c(N(c2ccc(-c3ccc4ccccc4c3)cc2)c2cc3ccccc3c3ccccc23)c([2H])c([2H])c1-c1cc2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1cc2ccccc2c2ccccc12]

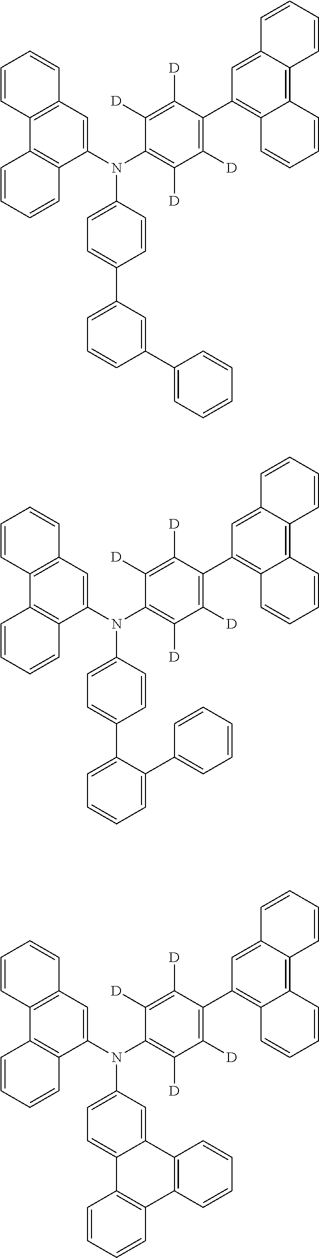 [SMILES: [2H]c1c([2H])c(N(c2ccc(-c3cccc(-c4ccccc4)c3)cc2)c2cc3ccccc3c3ccccc23)c([2H])c([2H])c1-c1cc2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1cc2ccccc2c2ccccc12 ; [2H]c1c([2H])c(N(c2ccc3c4ccccc4c4ccccc4c3c2)c2cc3ccccc3c3ccccc23)c([2H])c([2H])c1-c1cc2ccccc2c2ccccc12]

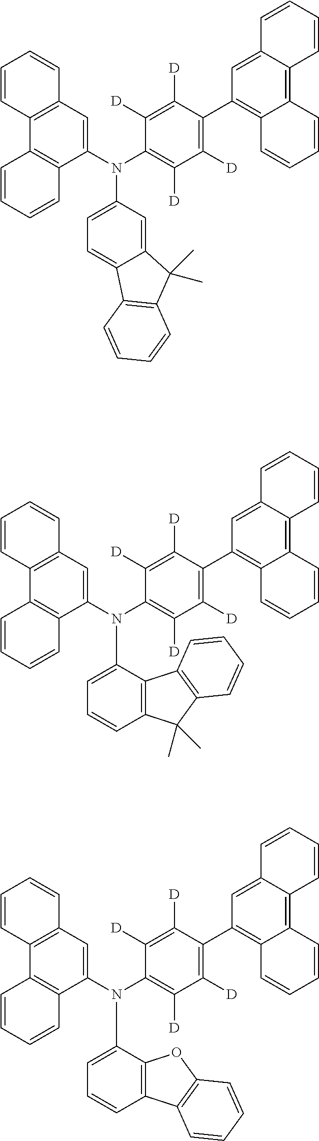 [SMILES: [2H]c1c([2H])c(N(c2ccc3c(c2)C(C)(C)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(C)C)c2cc3ccccc3c3ccccc23)c([2H])c([2H])c1-c1cc2ccccc2c2ccccc12 ; [2H]c1c([2H])c(N(c2cc3ccccc3c3ccccc23)c2cccc3c2oc2ccccc23)c([2H])c([2H])c1-c1cc2ccccc2c2ccccc12]

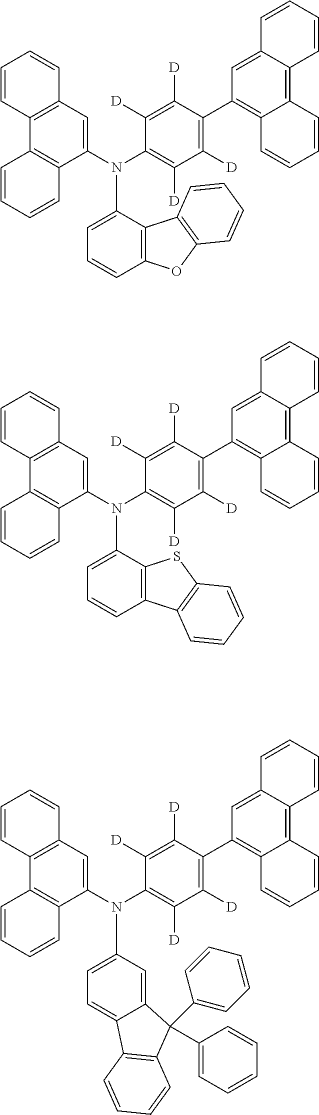 [SMILES: [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12 ; [2H]c1c([2H])c(N(c2ccc3c(c2)C(c2ccccc2)(c2ccccc2)c2ccccc2-3)c2cc3ccccc3c3ccccc23)c([2H])c([2H])c1-c1cc2ccccc2c2ccccc12]

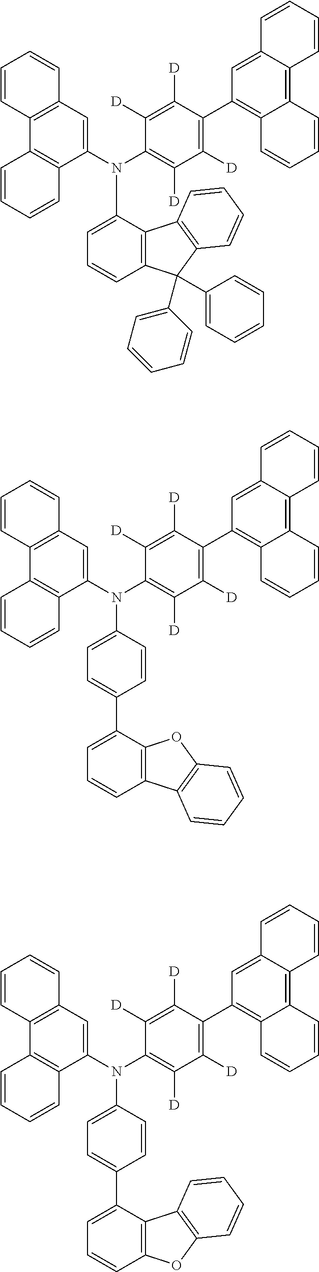 [SMILES: [2H]c1c([2H])c(N(c2cccc3c2-c2ccccc2C3(c2ccccc2)c2ccccc2)c2cc3ccccc3c3ccccc23)c([2H])c([2H])c1-c1cc2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1cc2ccccc2c2ccccc12]

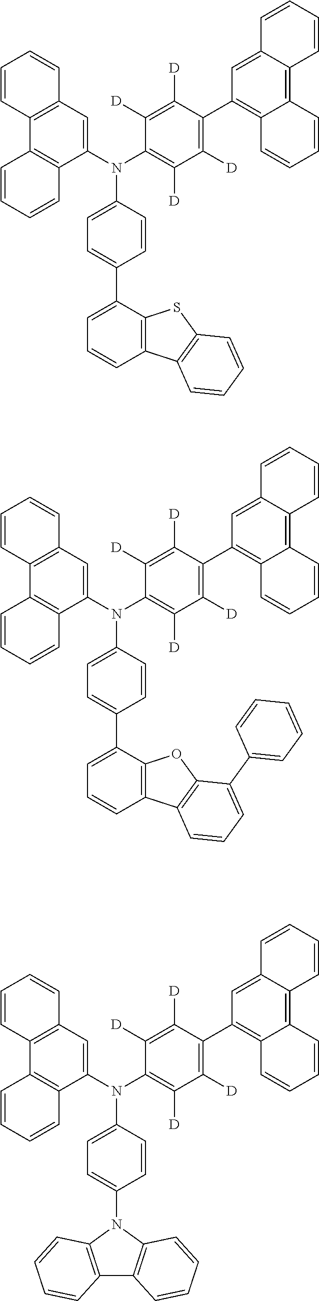 [SMILES: [2H]c1c([2H])c(N(c2ccc(-c3cccc4c3sc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1cc2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1cc2ccccc2c2ccccc12]

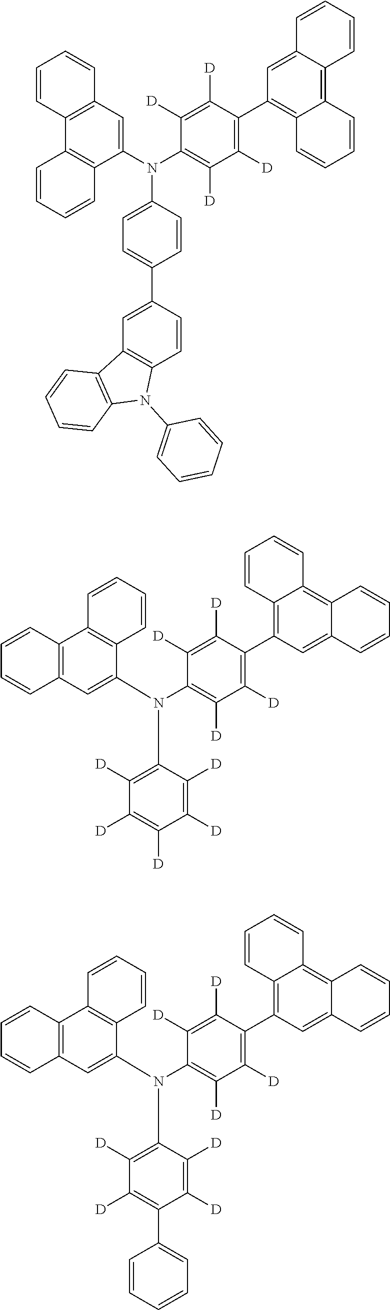 [SMILES: [2H]c1c([2H])c(N(c2ccc(-c3ccc4c(c3)c3ccccc3n4-c3ccccc3)cc2)c2cc3ccccc3c3ccccc23)c([2H])c([2H])c1-c1cc2ccccc2c2ccccc12 ; [2H]c1c([2H])c([2H])c(N(c2c([2H])c([2H])c(-c3cc4ccccc4c4ccccc34)c([2H])c2[2H])c2cc3ccccc3c3ccccc23)c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1]

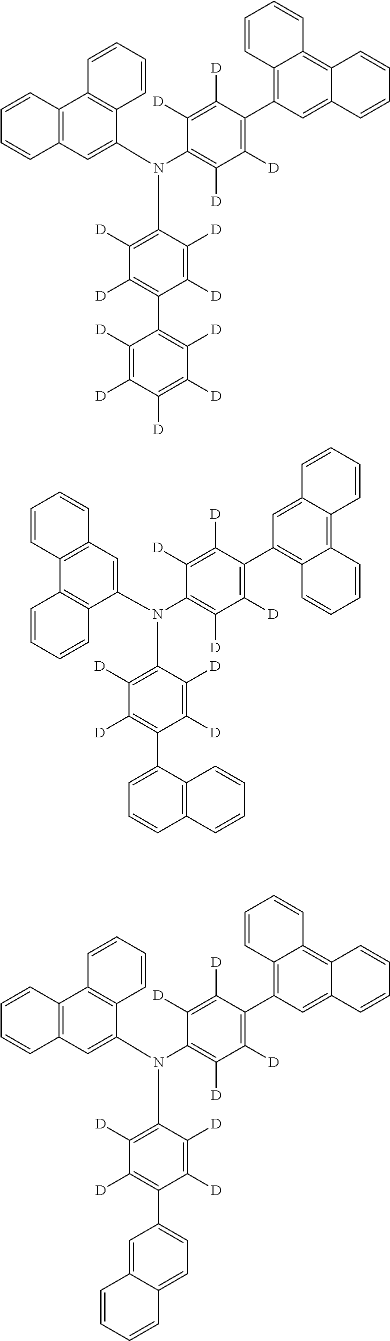 [SMILES: [2H]c1c([2H])c([2H])c(-c2c([2H])c([2H])c(N(c3c([2H])c([2H])c(-c4cc5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1]

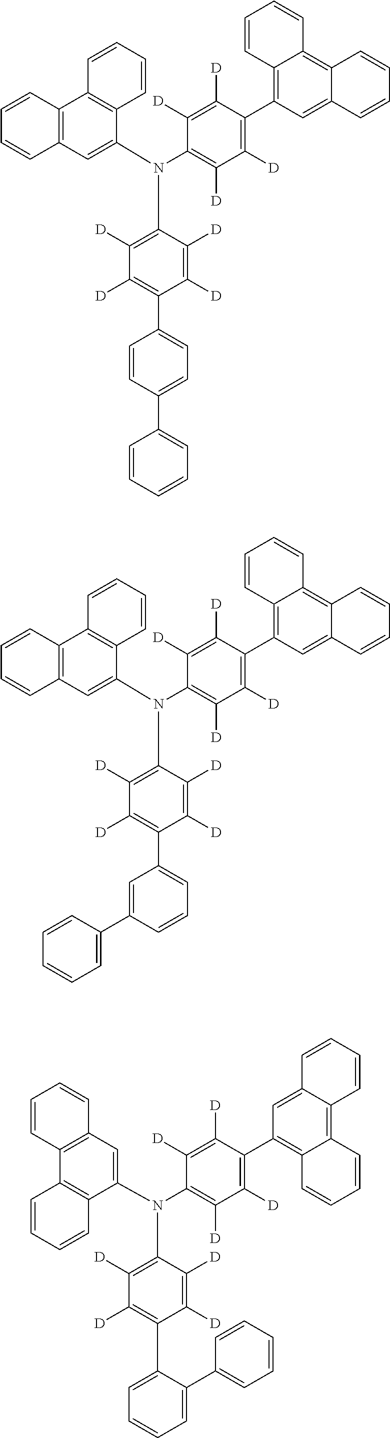 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1-c1ccccc1]

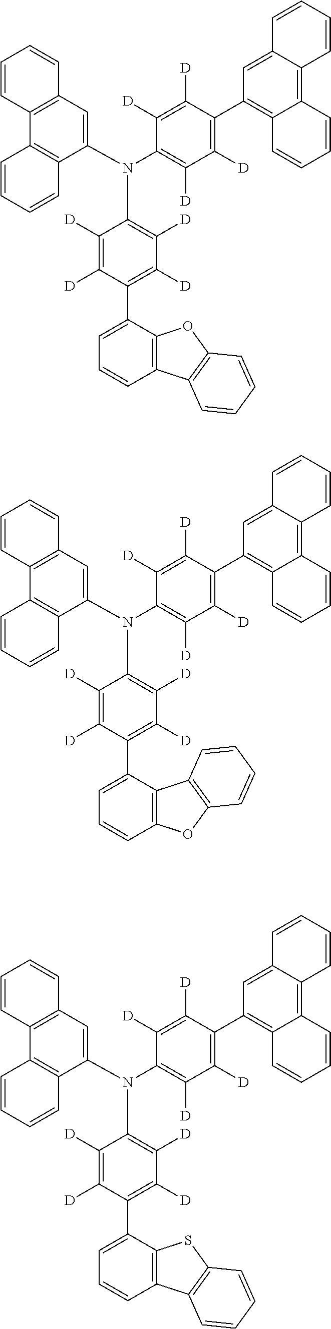 [SMILES: [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1cc2ccccc2c2ccccc12]

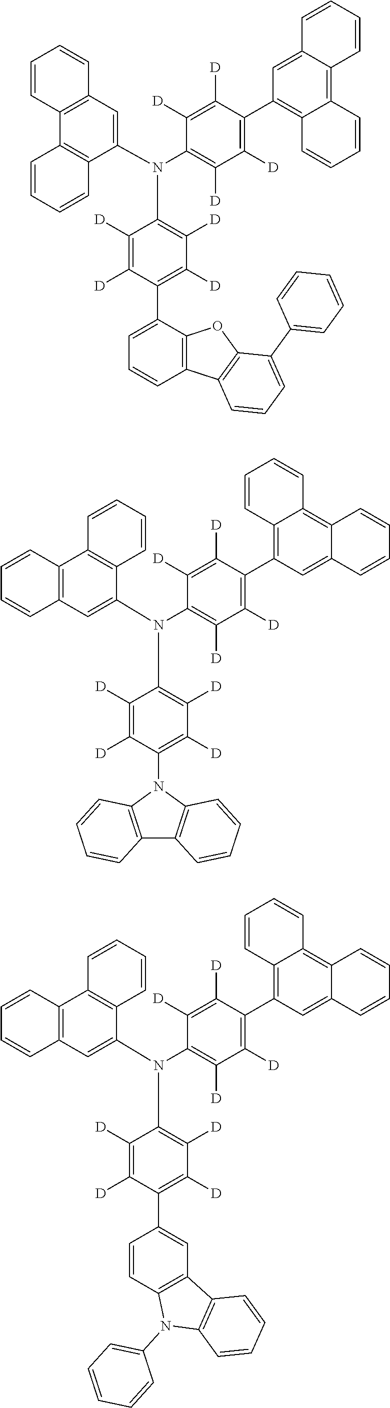

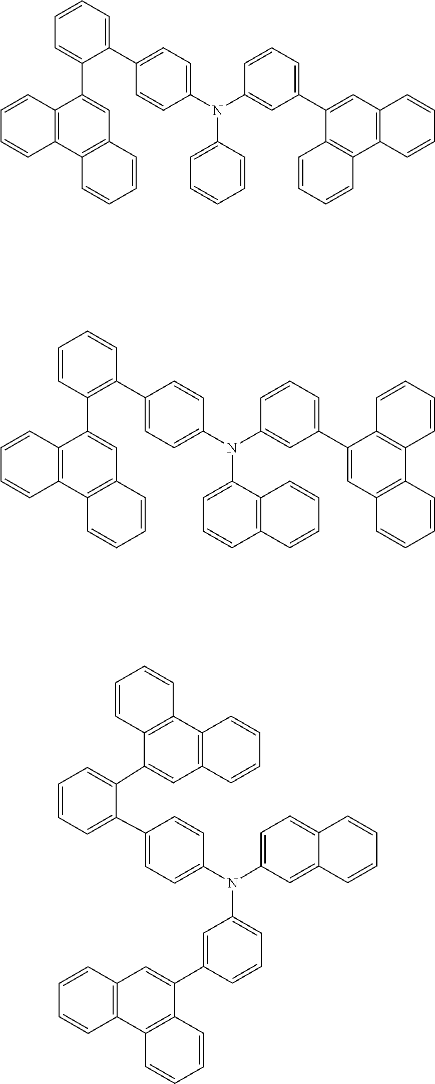 [SMILES: c1ccc(N(c2ccc(-c3ccccc3-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3ccccc3-c3cc4ccccc4c4ccccc34)cc2)c2cccc3ccccc23)c1 ; c1cc(-c2cc3ccccc3c3ccccc23)cc(N(c2ccc(-c3ccccc3-c3cc4ccccc4c4ccccc34)cc2)c2ccc3ccccc3c2)c1]

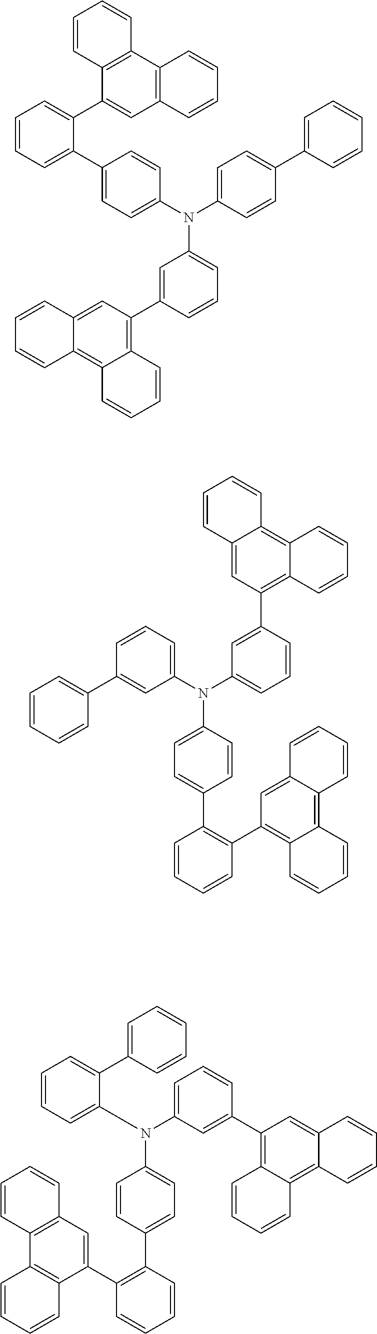 [SMILES: c1ccc(-c2ccc(N(c3ccc(-c4ccccc4-c4cc5ccccc5c5ccccc45)cc3)c3cccc(-c4cc5ccccc5c5ccccc45)c3)cc2)cc1 ; c1ccc(-c2cccc(N(c3ccc(-c4ccccc4-c4cc5ccccc5c5ccccc45)cc3)c3cccc(-c4cc5ccccc5c5ccccc45)c3)c2)cc1 ; c1ccc(-c2ccccc2N(c2ccc(-c3ccccc3-c3cc4ccccc4c4ccccc34)cc2)c2cccc(-c3cc4ccccc4c4ccccc34)c2)cc1]

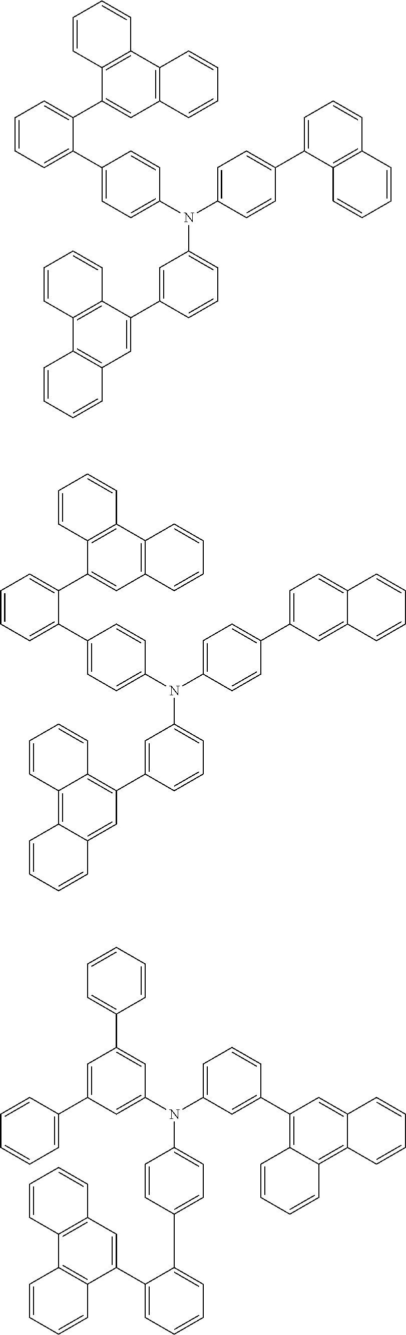 [SMILES: c1cc(-c2cc3ccccc3c3ccccc23)cc(N(c2ccc(-c3ccccc3-c3cc4ccccc4c4ccccc34)cc2)c2ccc(-c3cccc4ccccc34)cc2)c1 ; c1cc(-c2cc3ccccc3c3ccccc23)cc(N(c2ccc(-c3ccc4ccccc4c3)cc2)c2ccc(-c3ccccc3-c3cc4ccccc4c4ccccc34)cc2)c1 ; c1ccc(-c2cc(-c3ccccc3)cc(N(c3ccc(-c4ccccc4-c4cc5ccccc5c5ccccc45)cc3)c3cccc(-c4cc5ccccc5c5ccccc45)c3)c2)cc1]

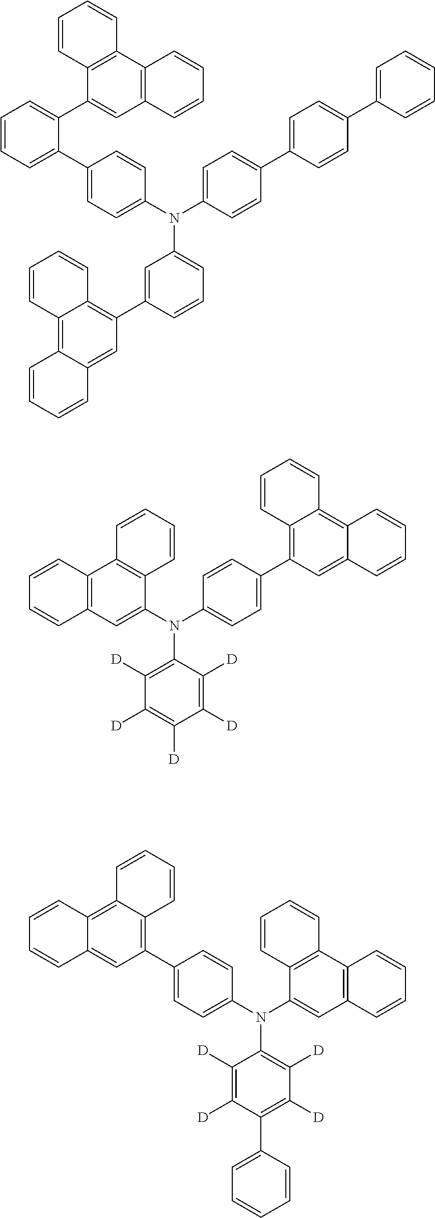 [SMILES: c1ccc(-c2ccc(-c3ccc(N(c4ccc(-c5ccccc5-c5cc6ccccc6c6ccccc56)cc4)c4cccc(-c5cc6ccccc6c6ccccc56)c4)cc3)cc2)cc1 ; [2H]c1c([2H])c([2H])c(N(c2ccc(-c3cc4ccccc4c4ccccc34)cc2)c2cc3ccccc3c3ccccc23)c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1ccccc1]

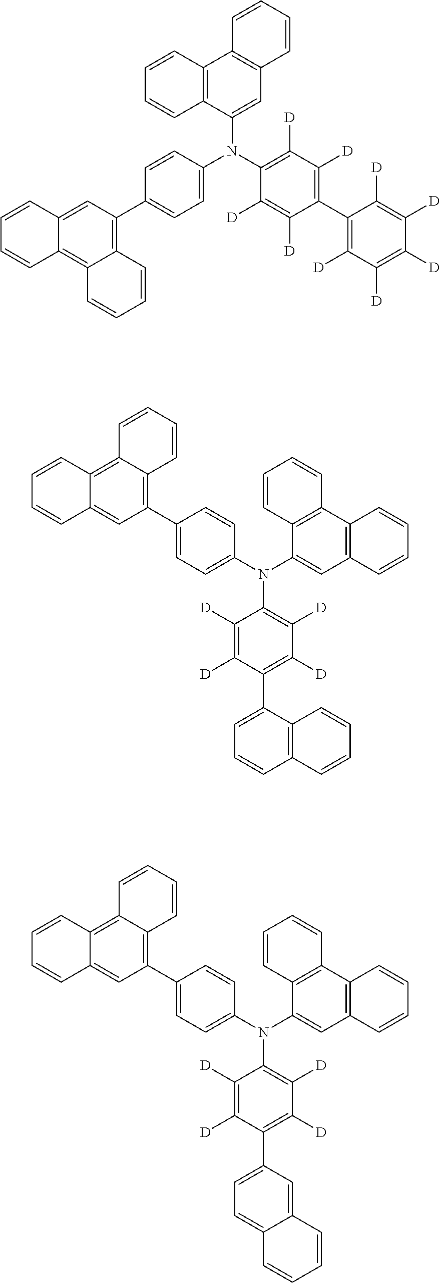 [SMILES: [2H]c1c([2H])c([2H])c(-c2c([2H])c([2H])c(N(c3ccc(-c4cc5ccccc5c5ccccc45)cc3)c3cc4ccccc4c4ccccc34)c([2H])c2[2H])c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1cccc2ccccc12 ; [2H]c1c([2H])c(N(c2ccc(-c3cc4ccccc4c4ccccc34)cc2)c2cc3ccccc3c3ccccc23)c([2H])c([2H])c1-c1ccc2ccccc2c1]

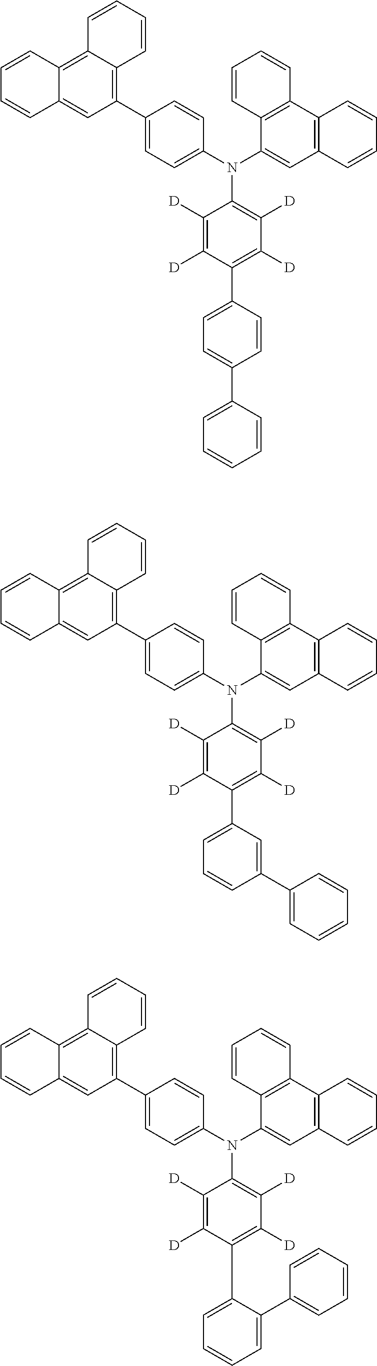 [SMILES: [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3cc4ccccc4c4ccccc34)cc2)c2cc3ccccc3c3ccccc23)c([2H])c([2H])c1-c1ccccc1-c1ccccc1]

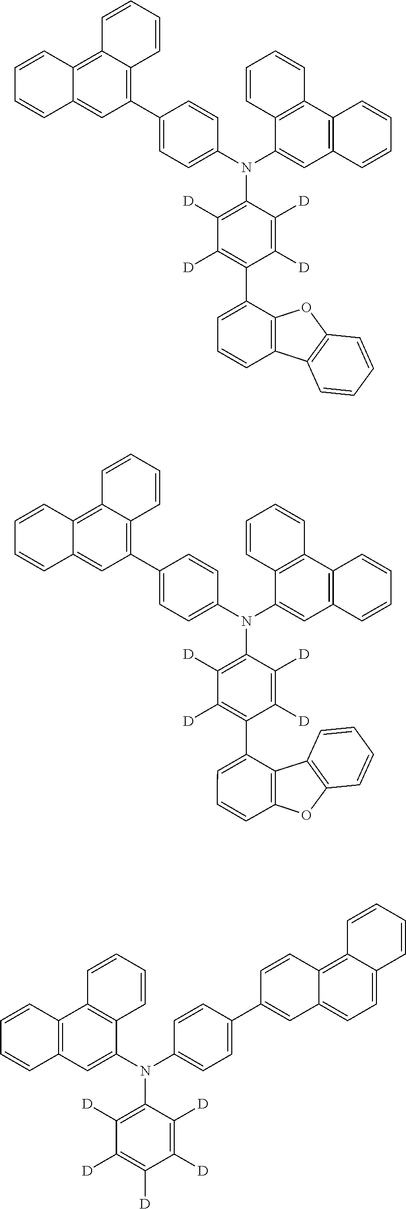 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3cc4ccccc4c4ccccc34)cc2)c2cc3ccccc3c3ccccc23)c([2H])c([2H])c1-c1cccc2oc3ccccc3c12 ; [2H]c1c([2H])c([2H])c(N(c2ccc(-c3ccc4c(ccc5ccccc54)c3)cc2)c2cc3ccccc3c3ccccc23)c([2H])c1[2H]]

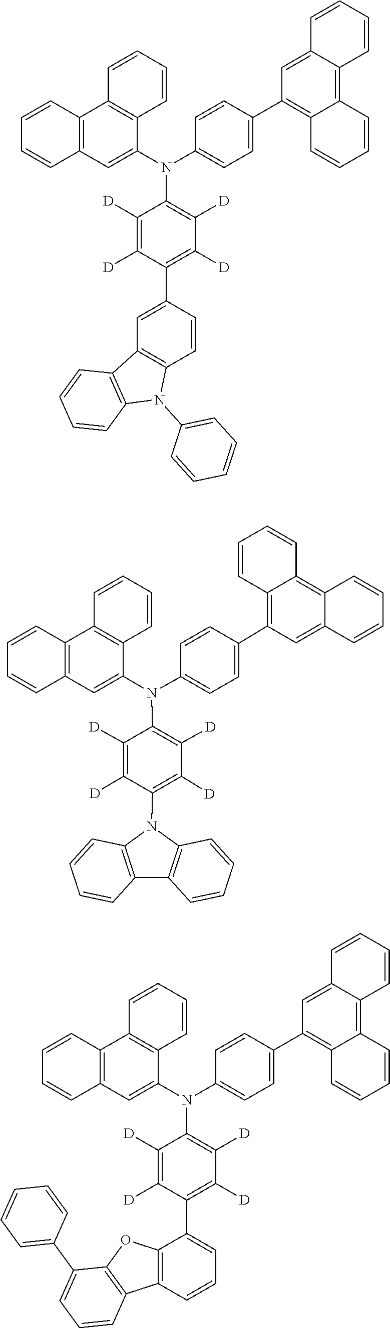 [SMILES: [2H]c1c([2H])c(N(c2ccc(-c3cc4ccccc4c4ccccc34)cc2)c2cc3ccccc3c3ccccc23)c([2H])c([2H])c1-c1ccc2c(c1)c1ccccc1n2-c1ccccc1 ; [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12 ; [2H]c1c([2H])c(N(c2ccc(-c3cc4ccccc4c4ccccc34)cc2)c2cc3ccccc3c3ccccc23)c([2H])c([2H])c1-c1cccc2c1oc1c(-c3ccccc3)cccc12]

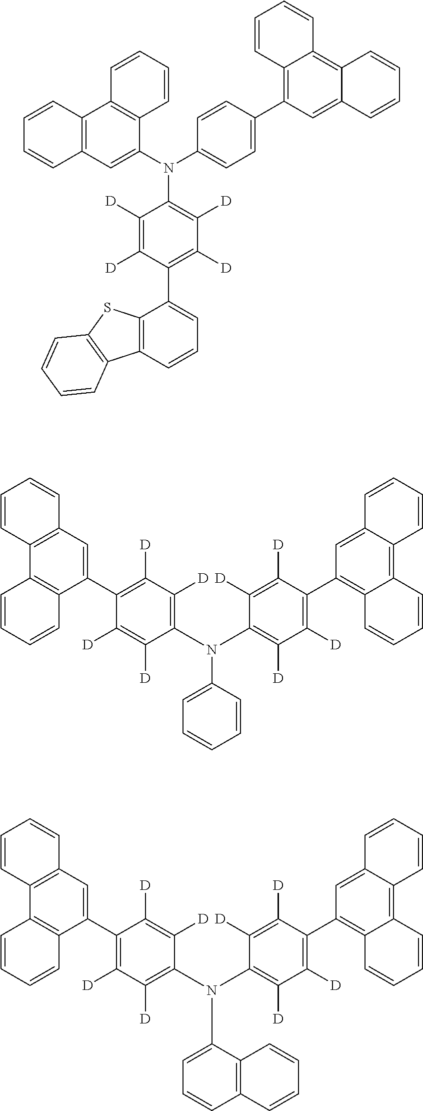 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1sc1ccccc12 ; [2H]c1c([2H])c(N(c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cccc3ccccc23)c([2H])c([2H])c1-c1cc2ccccc2c2ccccc12]

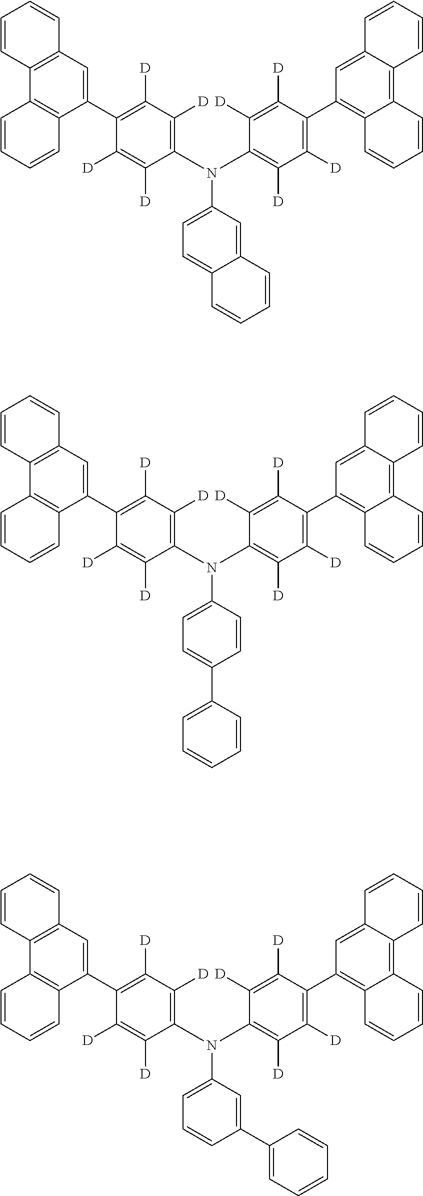 [SMILES: [2H]c1c([2H])c(N(c2ccc3ccccc3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccccc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2cccc(-c3ccccc3)c2)c2c([2H])c([2H])c(-c3cc4ccccc4c4ccccc34)c([2H])c2[2H])c([2H])c([2H])c1-c1cc2ccccc2c2ccccc12]

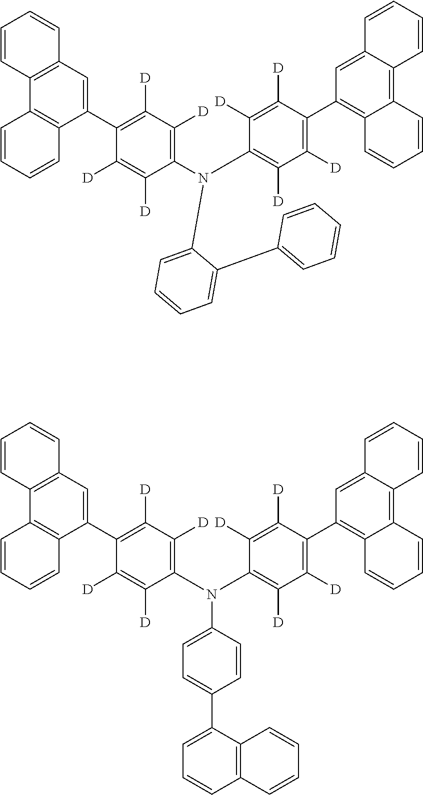 [SMILES: [2H]c1c([2H])c(N(c2ccccc2-c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12]

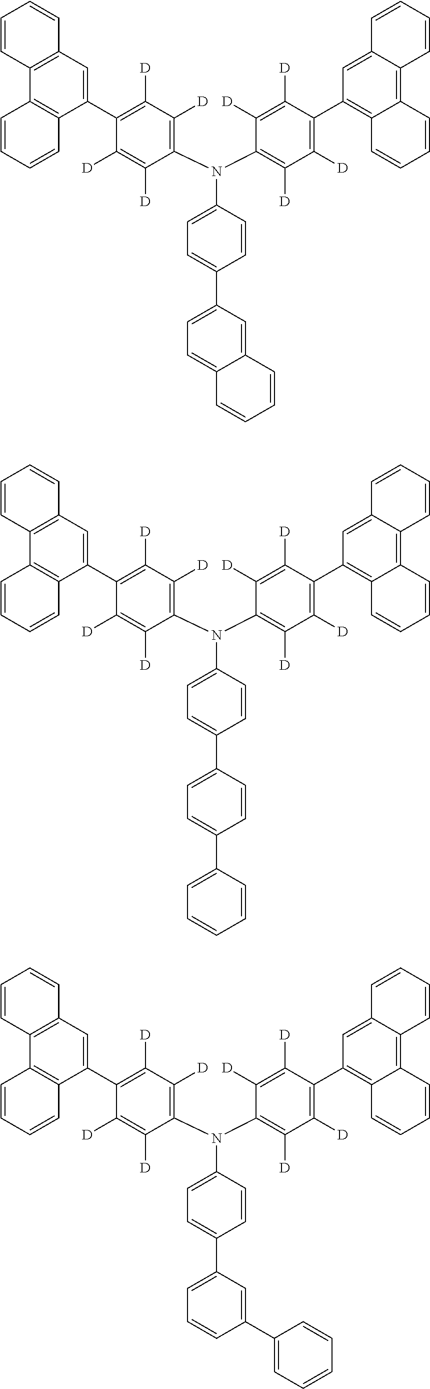 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc(-c4ccccc4)c3)cc2)c2c([2H])c([2H])c(-c3cc4ccccc4c4ccccc34)c([2H])c2[2H])c([2H])c([2H])c1-c1cc2ccccc2c2ccccc12]

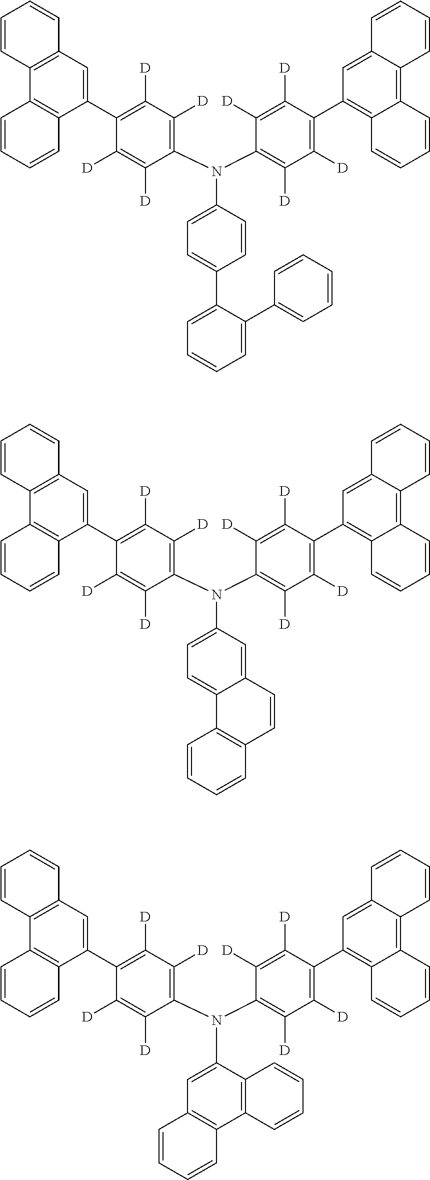 [SMILES: [2H]c1c([2H])c(N(c2ccc(-c3ccccc3-c3ccccc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c(ccc4ccccc43)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cc2ccccc2c2ccccc12]

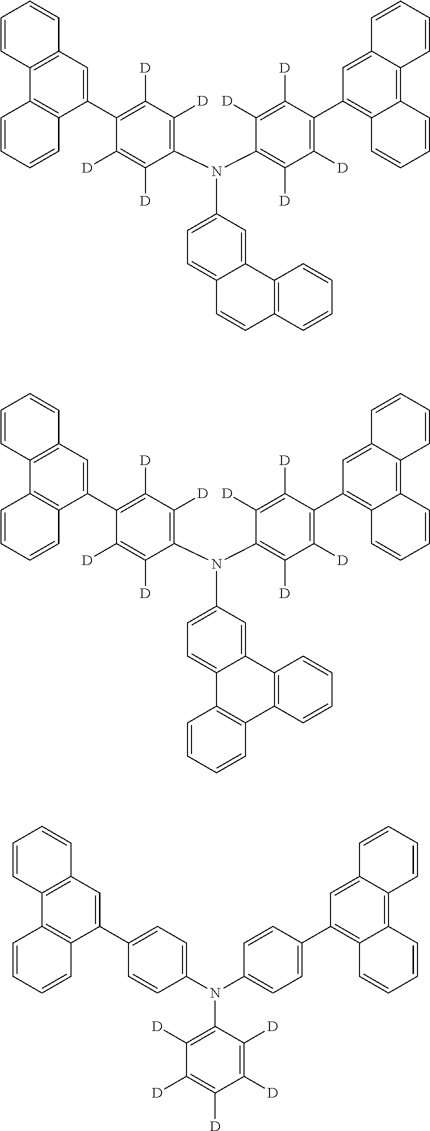 [SMILES: [2H]c1c([2H])c(N(c2ccc3ccc4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c4ccccc4c4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c([2H])c(N(c2ccc(-c3cc4ccccc4c4ccccc34)cc2)c2ccc(-c3cc4ccccc4c4ccccc34)cc2)c([2H])c1[2H]]

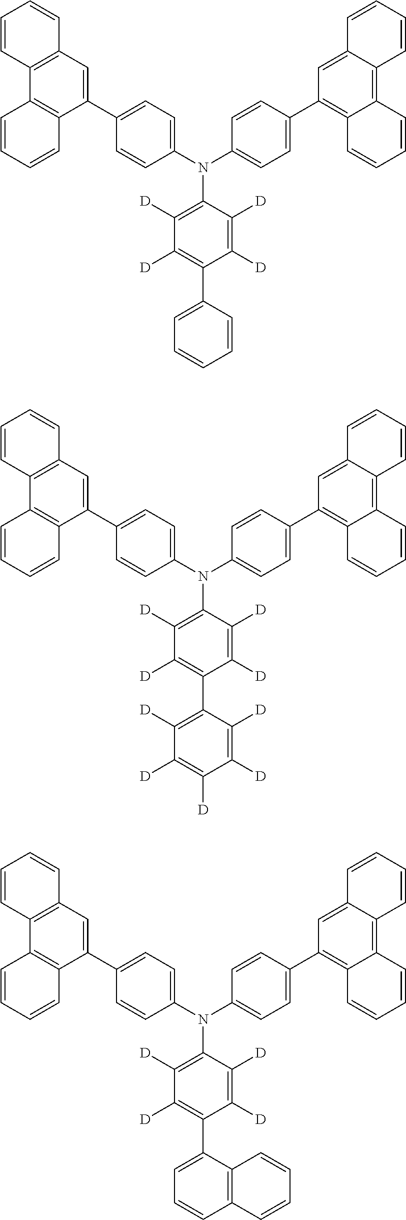 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3ccc(-c4cc5ccccc5c5ccccc45)cc3)c([2H])c2[2H])c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1cccc2ccccc12]

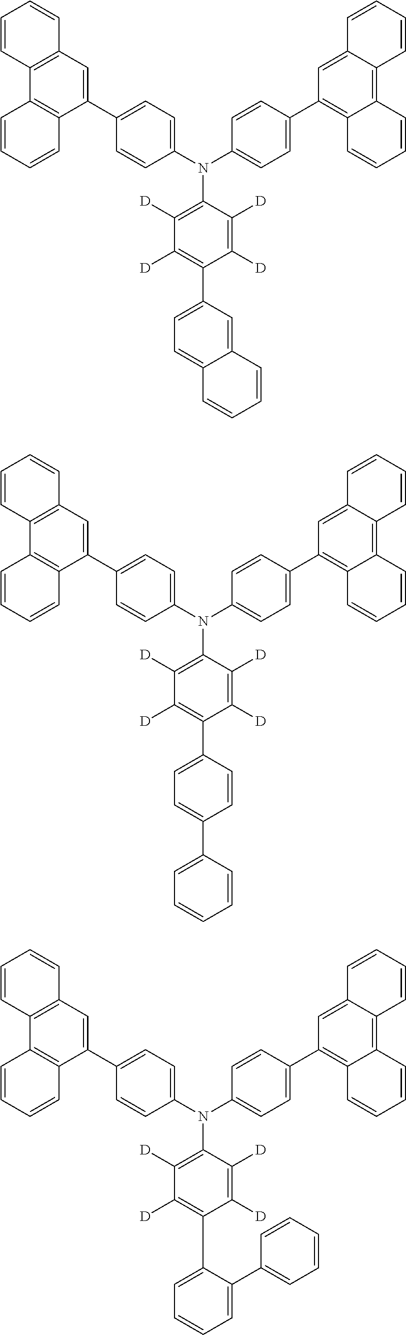 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc2ccccc2c1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc(-c2ccccc2)cc1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1-c1ccccc1]

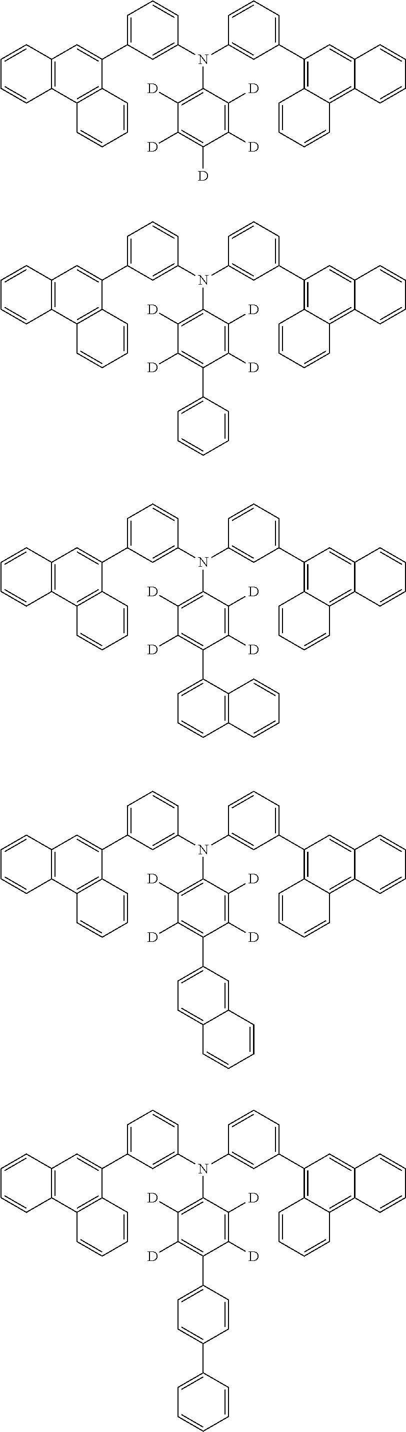

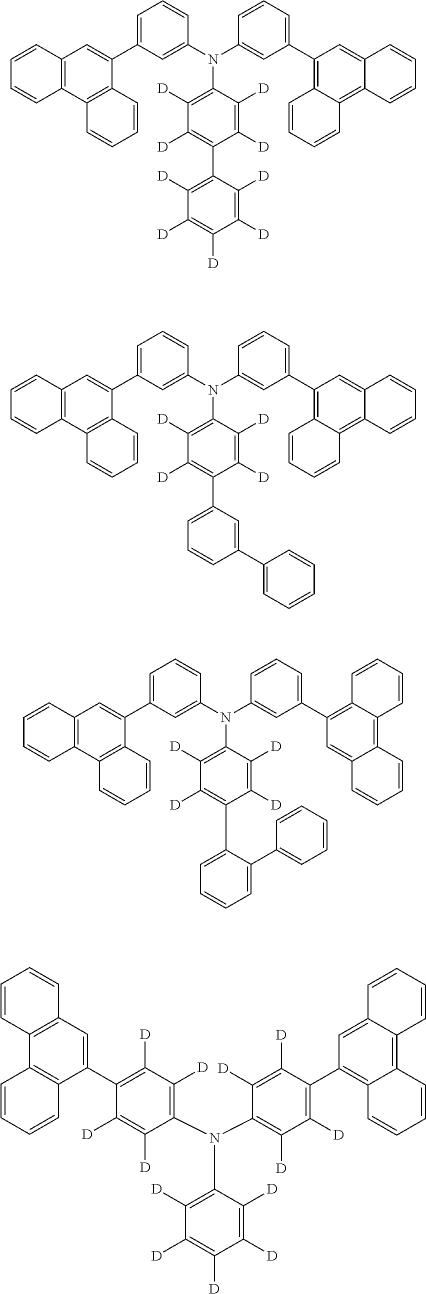

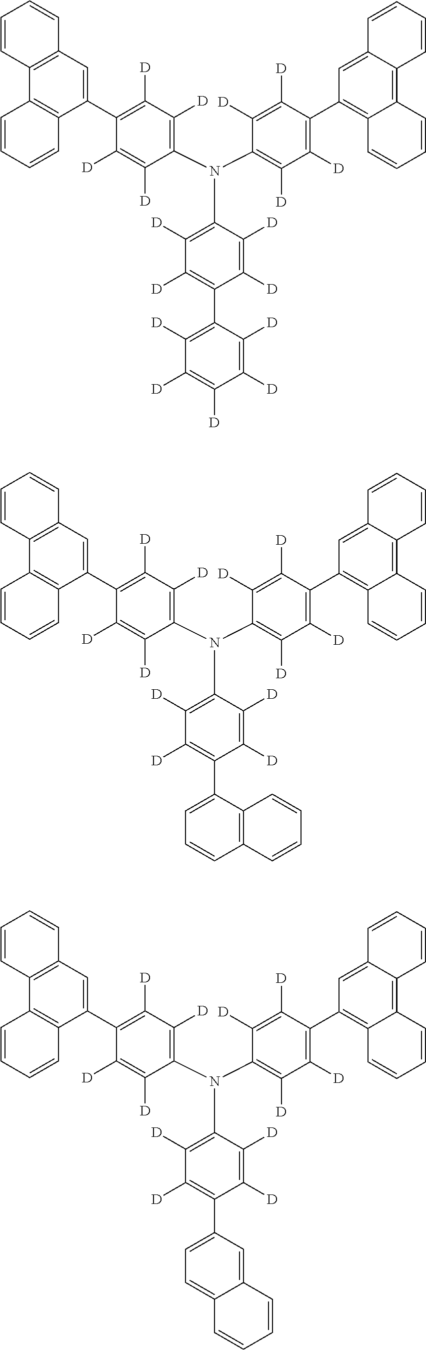 [SMILES: [2H]c1c([2H])c([2H])c(-c2c([2H])c([2H])c(N(c3c([2H])c([2H])c(-c4cc5ccccc5c5ccccc45)c([2H])c3[2H])c3c([2H])c([2H])c(-c4cc5ccccc5c5ccccc45)c([2H])c3[2H])c([2H])c2[2H])c([2H])c1[2H] ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1cccc2ccccc12 ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccc2ccccc2c1]

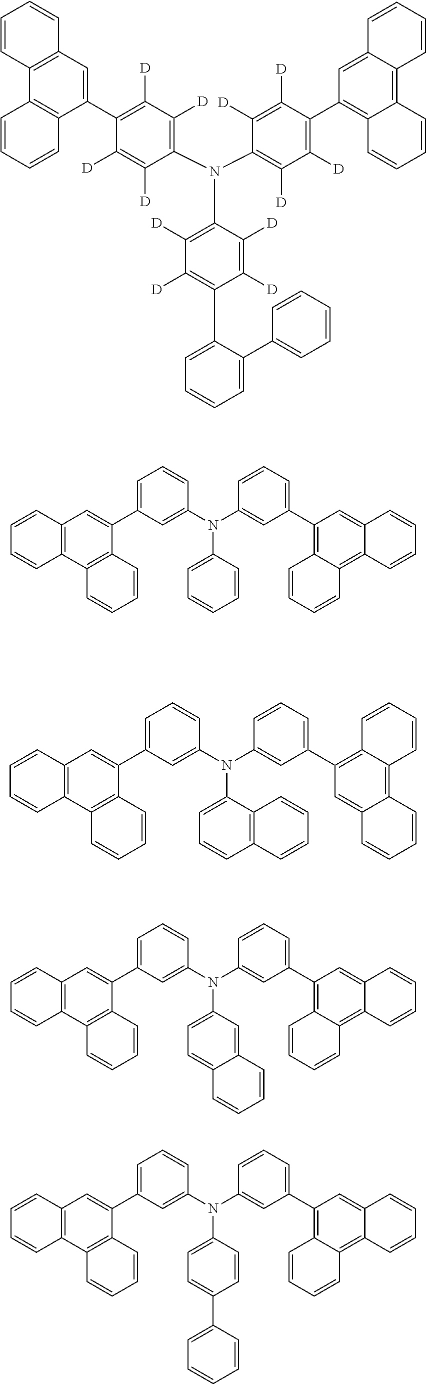 [SMILES: [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccccc1-c1ccccc1 ; c1ccc(N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2cccc(-c3cc4ccccc4c4ccccc34)c2)c2cccc3ccccc23)c1 ; c1cc(-c2cc3ccccc3c3ccccc23)cc(N(c2cccc(-c3cc4ccccc4c4ccccc34)c2)c2ccc3ccccc3c2)c1 ; c1ccc(-c2ccc(N(c3cccc(-c4cc5ccccc5c5ccccc45)c3)c3cccc(-c4cc5ccccc5c5ccccc45)c3)cc2)cc1]

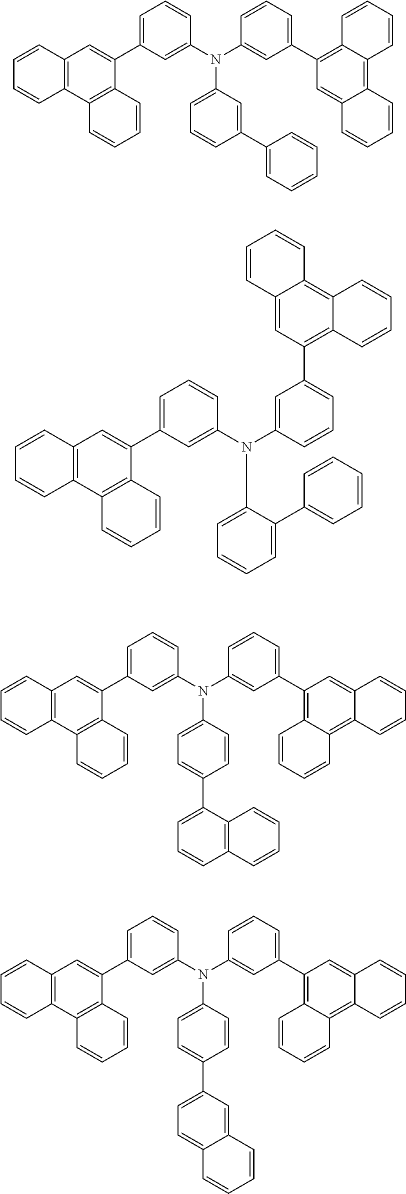 [SMILES: c1ccc(-c2cccc(N(c3cccc(-c4cc5ccccc5c5ccccc45)c3)c3cccc(-c4cc5ccccc5c5ccccc45)c3)c2)cc1 ; c1ccc(-c2ccccc2N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2cccc(-c3cc4ccccc4c4ccccc34)c2)c1 ; c1cc(-c2cc3ccccc3c3ccccc23)cc(N(c2ccc(-c3ccc4ccccc4c3)cc2)c2cccc(-c3cc4ccccc4c4ccccc34)c2)c1]

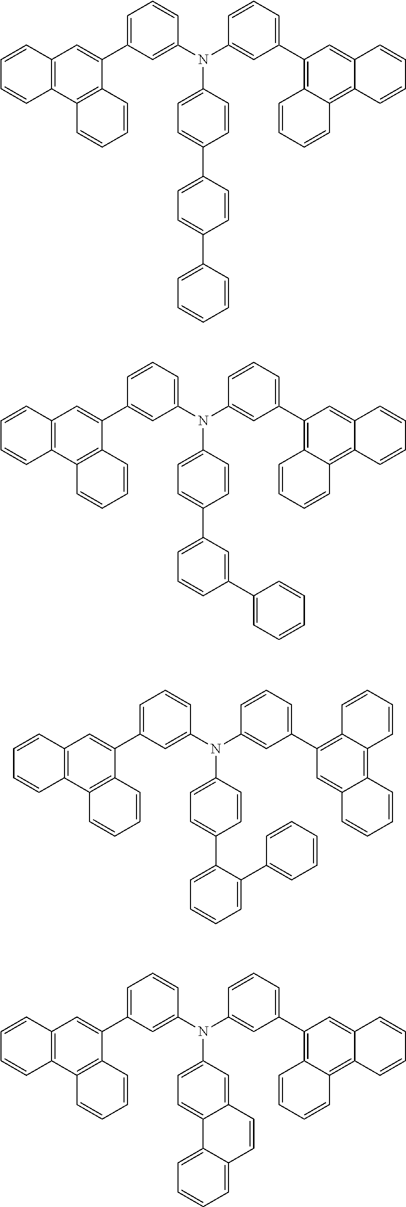 [SMILES: c1ccc(-c2ccc(-c3ccc(N(c4cccc(-c5cc6ccccc6c6ccccc56)c4)c4cccc(-c5cc6ccccc6c6ccccc56)c4)cc3)cc2)cc1 ; c1ccc(-c2cccc(-c3ccc(N(c4cccc(-c5cc6ccccc6c6ccccc56)c4)c4cccc(-c5cc6ccccc6c6ccccc56)c4)cc3)c2)cc1 ; c1ccc(-c2ccccc2-c2ccc(N(c3cccc(-c4cc5ccccc5c5ccccc45)c3)c3cccc(-c4cc5ccccc5c5ccccc45)c3)cc2)cc1 ; c1cc(-c2cc3ccccc3c3ccccc23)cc(N(c2cccc(-c3cc4ccccc4c4ccccc34)c2)c2ccc3c(ccc4ccccc43)c2)c1]

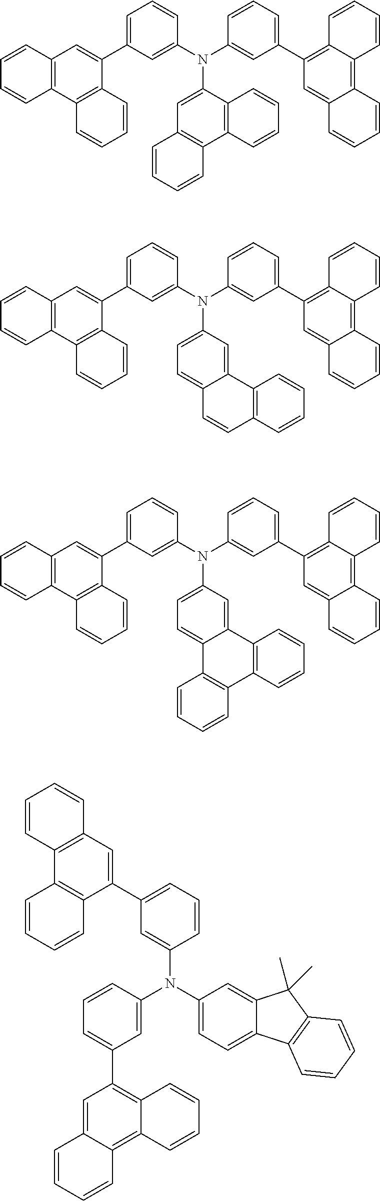 [SMILES: c1cc(-c2cc3ccccc3c3ccccc23)cc(N(c2cccc(-c3cc4ccccc4c4ccccc34)c2)c2cc3ccccc3c3ccccc23)c1 ; c1cc(-c2cc3ccccc3c3ccccc23)cc(N(c2cccc(-c3cc4ccccc4c4ccccc34)c2)c2ccc3ccc4ccccc4c3c2)c1 ; c1cc(-c2cc3ccccc3c3ccccc23)cc(N(c2cccc(-c3cc4ccccc4c4ccccc34)c2)c2ccc3c4ccccc4c4ccccc4c3c2)c1 ; CC1(C)c2ccccc2-c2ccc(N(c3cccc(-c4cc5ccccc5c5ccccc45)c3)c3cccc(-c4cc5ccccc5c5ccccc45)c3)cc21]

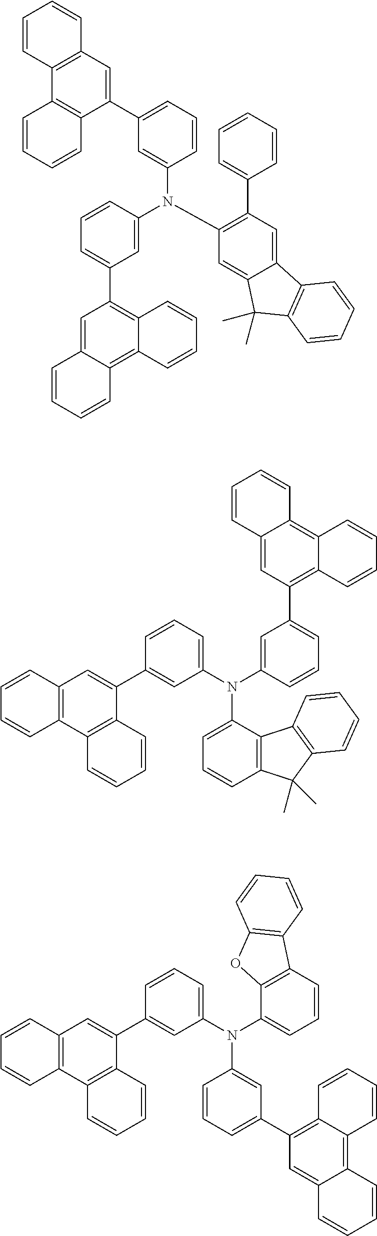 [SMILES: CC1(C)c2ccccc2-c2cc(-c3ccccc3)c(N(c3cccc(-c4cc5ccccc5c5ccccc45)c3)c3cccc(-c4cc5ccccc5c5ccccc45)c3)cc21 ; CC1(C)c2ccccc2-c2c(N(c3cccc(-c4cc5ccccc5c5ccccc45)c3)c3cccc(-c4cc5ccccc5c5ccccc45)c3)cccc21 ; c1cc(-c2cc3ccccc3c3ccccc23)cc(N(c2cccc(-c3cc4ccccc4c4ccccc34)c2)c2cccc3c2oc2ccccc23)c1]

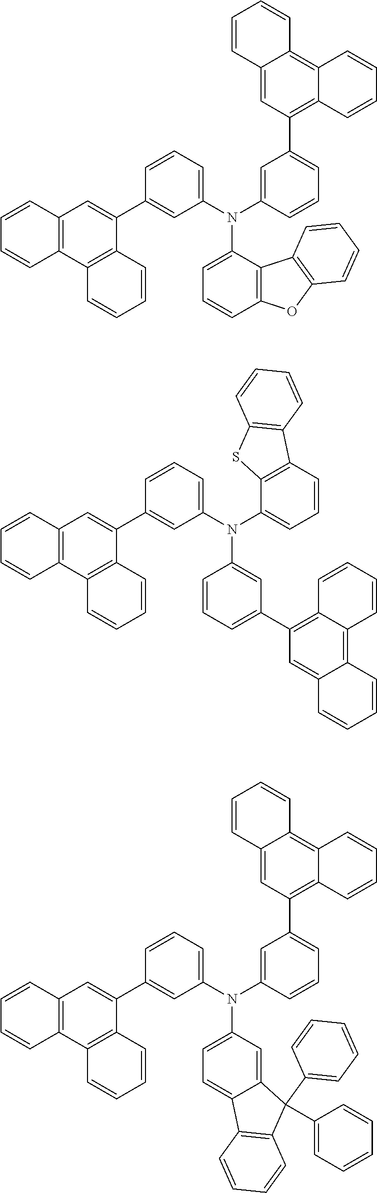 [SMILES: c1cc(-c2cc3ccccc3c3ccccc23)cc(N(c2cccc(-c3cc4ccccc4c4ccccc34)c2)c2cccc3oc4ccccc4c23)c1 ; c1cc(-c2cc3ccccc3c3ccccc23)cc(N(c2cccc(-c3cc4ccccc4c4ccccc34)c2)c2cccc3c2sc2ccccc23)c1 ; c1ccc(C2(c3ccccc3)c3ccccc3-c3ccc(N(c4cccc(-c5cc6ccccc6c6ccccc56)c4)c4cccc(-c5cc6ccccc6c6ccccc56)c4)cc32)cc1]

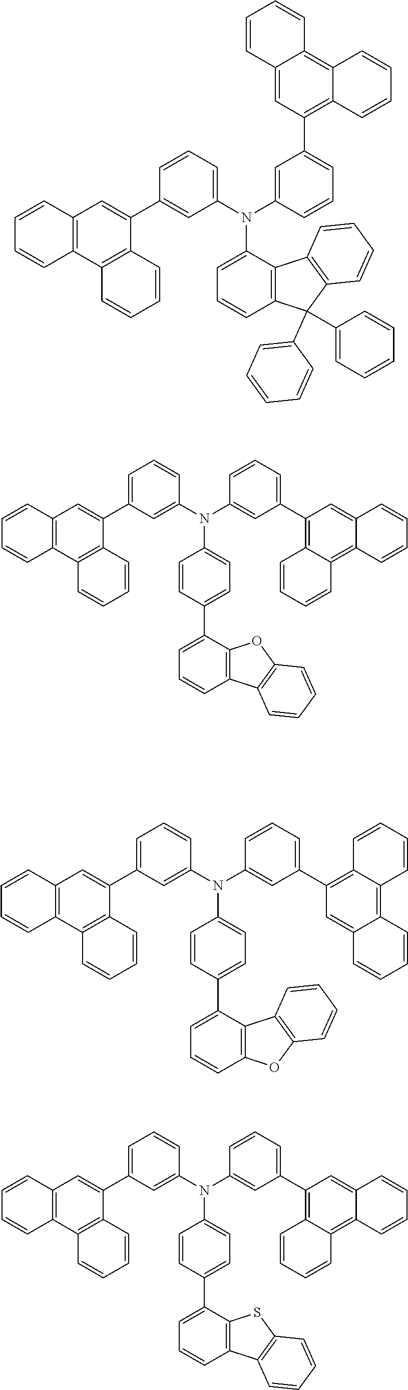 [SMILES: c1ccc(C2(c3ccccc3)c3ccccc3-c3c(N(c4cccc(-c5cc6ccccc6c6ccccc56)c4)c4cccc(-c5cc6ccccc6c6ccccc56)c4)cccc32)cc1 ; c1cc(-c2cc3ccccc3c3ccccc23)cc(N(c2ccc(-c3cccc4c3oc3ccccc34)cc2)c2cccc(-c3cc4ccccc4c4ccccc34)c2)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1 ; c1cc(-c2cc3ccccc3c3ccccc23)cc(N(c2ccc(-c3cccc4c3sc3ccccc34)cc2)c2cccc(-c3cc4ccccc4c4ccccc34)c2)c1]

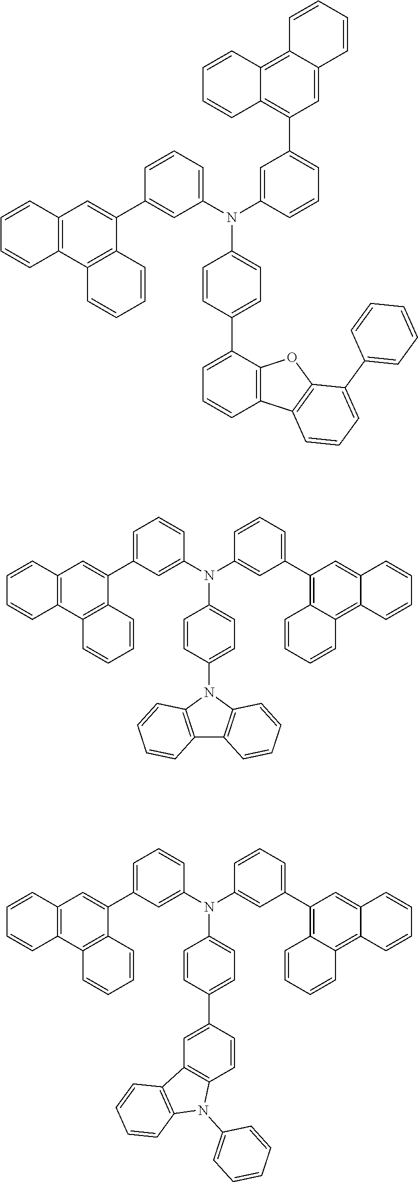 [SMILES: c1ccc(-c2cccc3c2oc2c(-c4ccc(N(c5cccc(-c6cc7ccccc7c7ccccc67)c5)c5cccc(-c6cc7ccccc7c7ccccc67)c5)cc4)cccc23)cc1 ; c1cc(-c2cc3ccccc3c3ccccc23)cc(N(c2ccc(-n3c4ccccc4c4ccccc43)cc2)c2cccc(-c3cc4ccccc4c4ccccc34)c2)c1 ; c1ccc(-n2c3ccccc3c3cc(-c4ccc(N(c5cccc(-c6cc7ccccc7c7ccccc67)c5)c5cccc(-c6cc7ccccc7c7ccccc67)c5)cc4)ccc32)cc1]

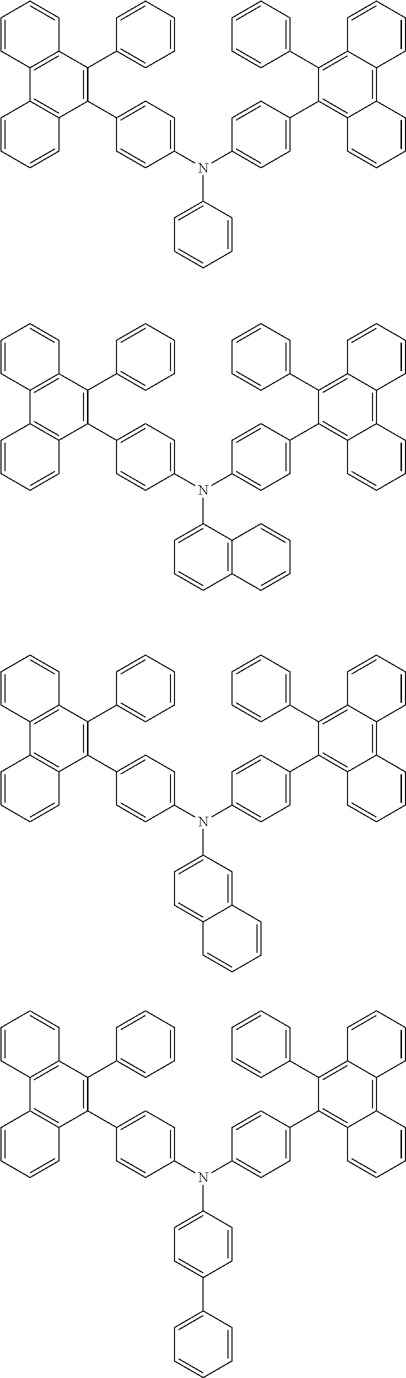 [SMILES: c1ccc(-c2c(-c3ccc(N(c4ccccc4)c4ccc(-c5c(-c6ccccc6)c6ccccc6c6ccccc56)cc4)cc3)c3ccccc3c3ccccc23)cc1 ; c1ccc(-c2c(-c3ccc(N(c4ccc(-c5c(-c6ccccc6)c6ccccc6c6ccccc56)cc4)c4cccc5ccccc45)cc3)c3ccccc3c3ccccc23)cc1 ; c1ccc(-c2c(-c3ccc(N(c4ccc(-c5c(-c6ccccc6)c6ccccc6c6ccccc56)cc4)c4ccc5ccccc5c4)cc3)c3ccccc3c3ccccc23)cc1 ; c1ccc(-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1]

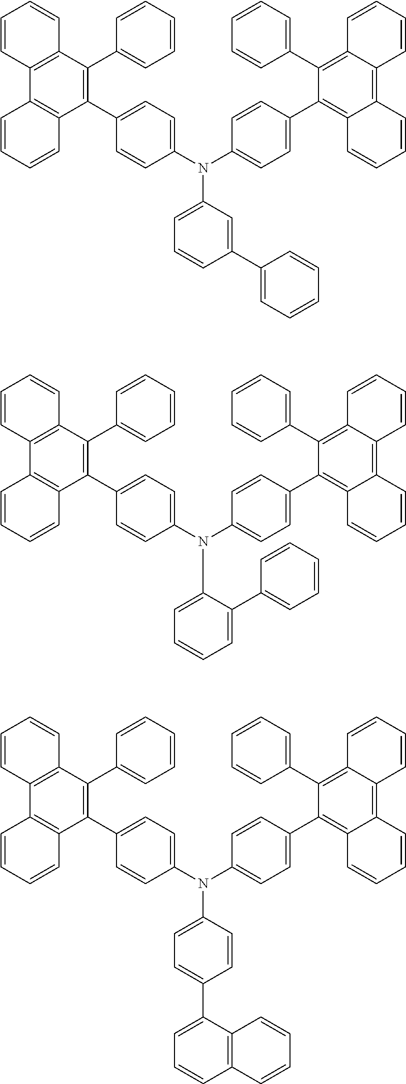 [SMILES: c1ccc(-c2cccc(N(c3ccc(-c4c(-c5ccccc5)c5ccccc5c5ccccc45)cc3)c3ccc(-c4c(-c5ccccc5)c5ccccc5c5ccccc45)cc3)c2)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1]

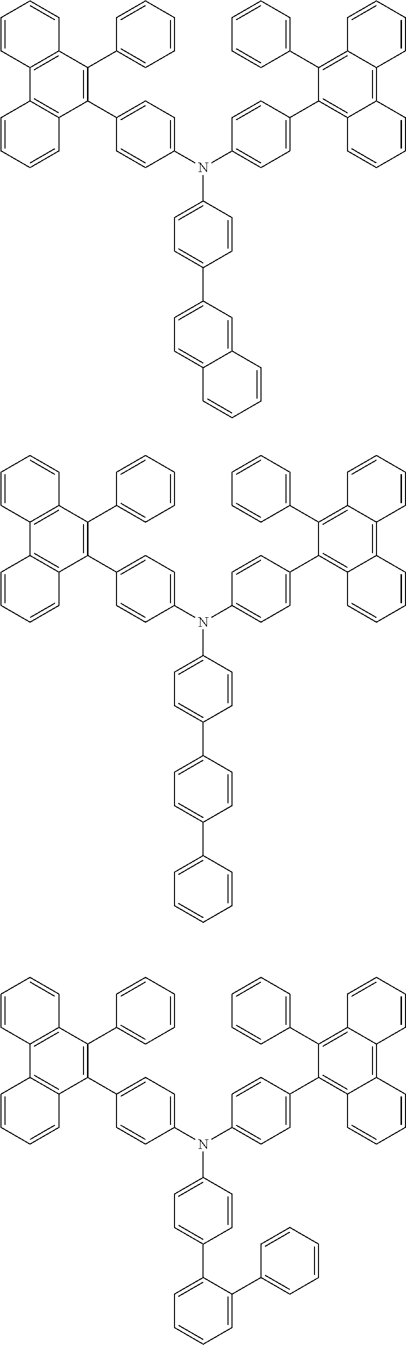 [SMILES: c1ccc(-c2c(-c3ccc(N(c4ccc(-c5ccc6ccccc6c5)cc4)c4ccc(-c5c(-c6ccccc6)c6ccccc6c6ccccc56)cc4)cc3)c3ccccc3c3ccccc23)cc1 ; c1ccc(-c2ccc(-c3ccc(N(c4ccc(-c5c(-c6ccccc6)c6ccccc6c6ccccc56)cc4)c4ccc(-c5c(-c6ccccc6)c6ccccc6c6ccccc56)cc4)cc3)cc2)cc1 ; c1ccc(-c2ccccc2-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1]

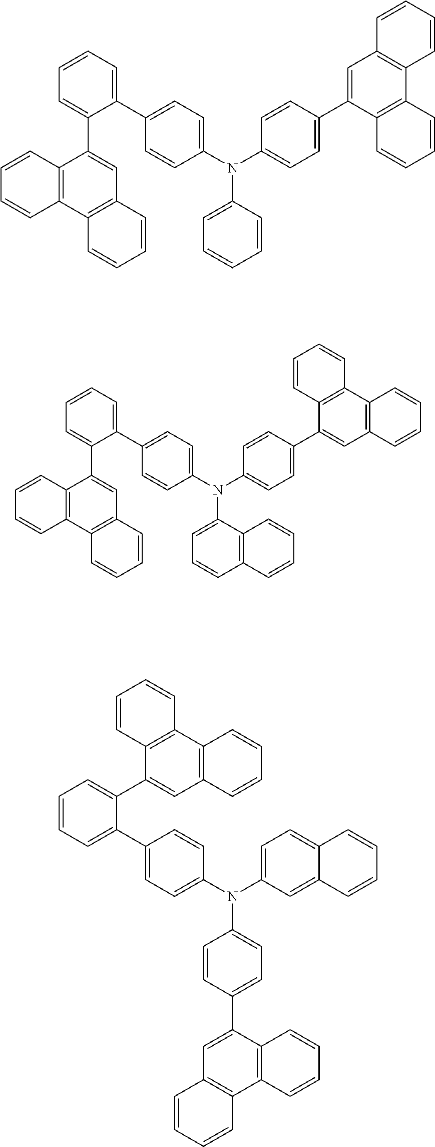 [SMILES: c1ccc(N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cc5ccccc5c5ccccc45)cc3)c3cccc4ccccc34)cc2)c1 ; c1ccc(-c2cc3ccccc3c3ccccc23)c(-c2ccc(N(c3ccc(-c4cc5ccccc5c5ccccc45)cc3)c3ccc4ccccc4c3)cc2)c1]

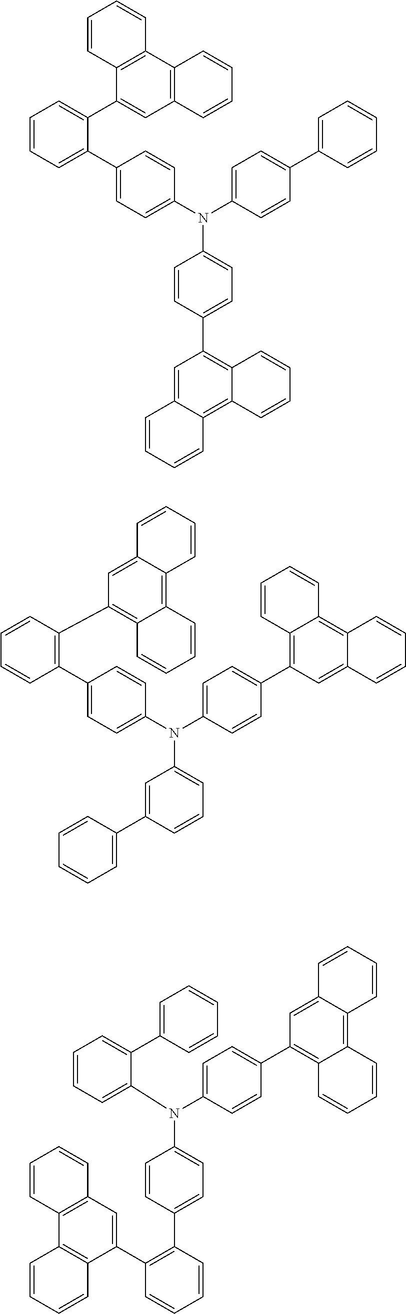

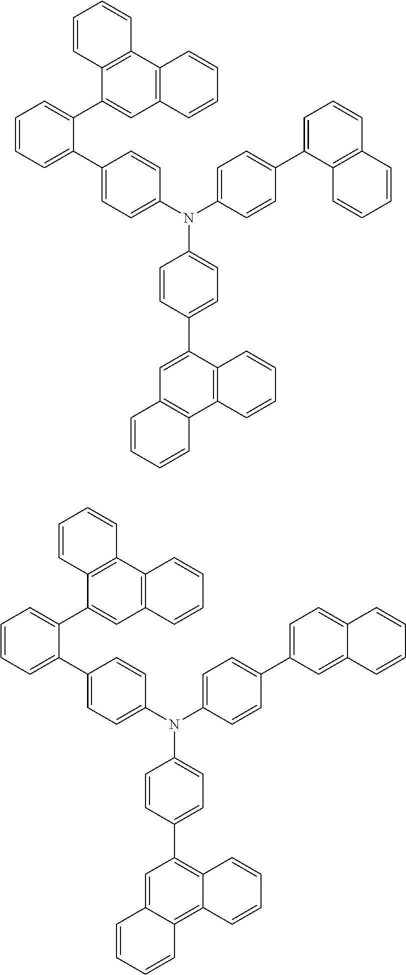 [SMILES: c1ccc(-c2cc3ccccc3c3ccccc23)c(-c2ccc(N(c3ccc(-c4cccc5ccccc45)cc3)c3ccc(-c4cc5ccccc5c5ccccc45)cc3)cc2)c1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1]

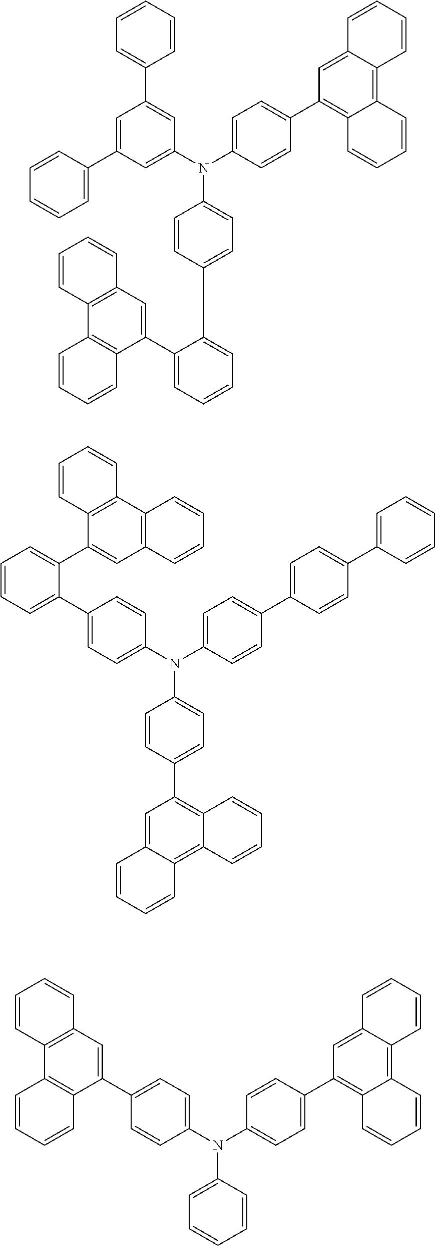

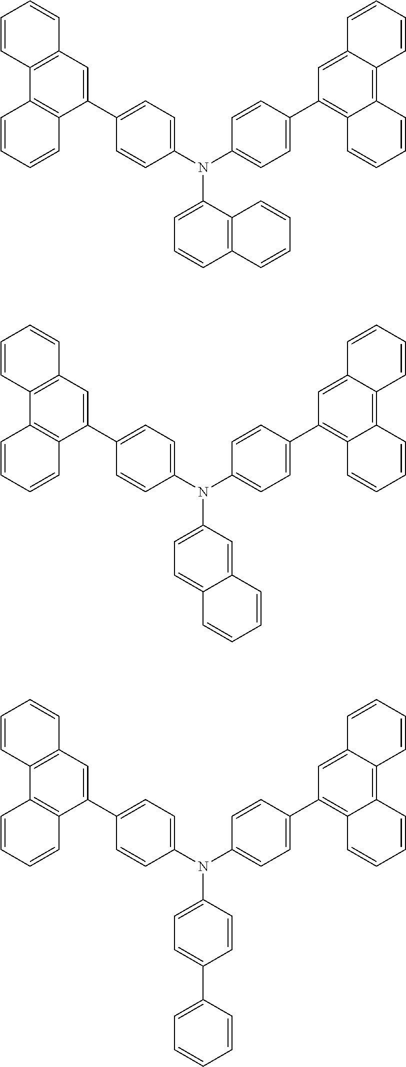 [SMILES: c1ccc2c(N(c3ccc(-c4cc5ccccc5c5ccccc45)cc3)c3ccc(-c4cc5ccccc5c5ccccc45)cc3)cccc2c1 ; c1ccc2cc(N(c3ccc(-c4cc5ccccc5c5ccccc45)cc3)c3ccc(-c4cc5ccccc5c5ccccc45)cc3)ccc2c1 ; c1ccc(-c2ccc(N(c3ccc(-c4cc5ccccc5c5ccccc45)cc3)c3ccc(-c4cc5ccccc5c5ccccc45)cc3)cc2)cc1]

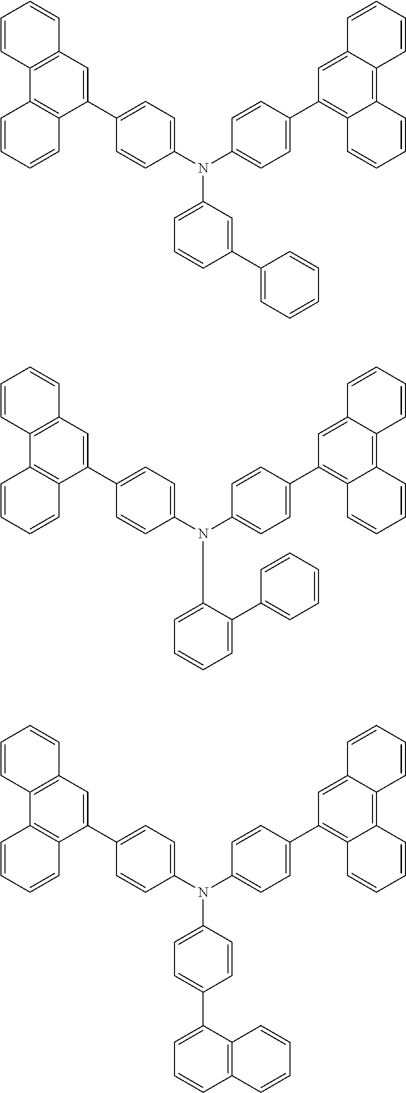 [SMILES: c1ccc(-c2cccc(N(c3ccc(-c4cc5ccccc5c5ccccc45)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cc4ccccc4c4ccccc34)cc2)cc1 ; c1ccc2c(-c3ccc(N(c4ccc(-c5cc6ccccc6c6ccccc56)cc4)c4ccc(-c5cc6ccccc6c6ccccc56)cc4)cc3)cccc2c1]

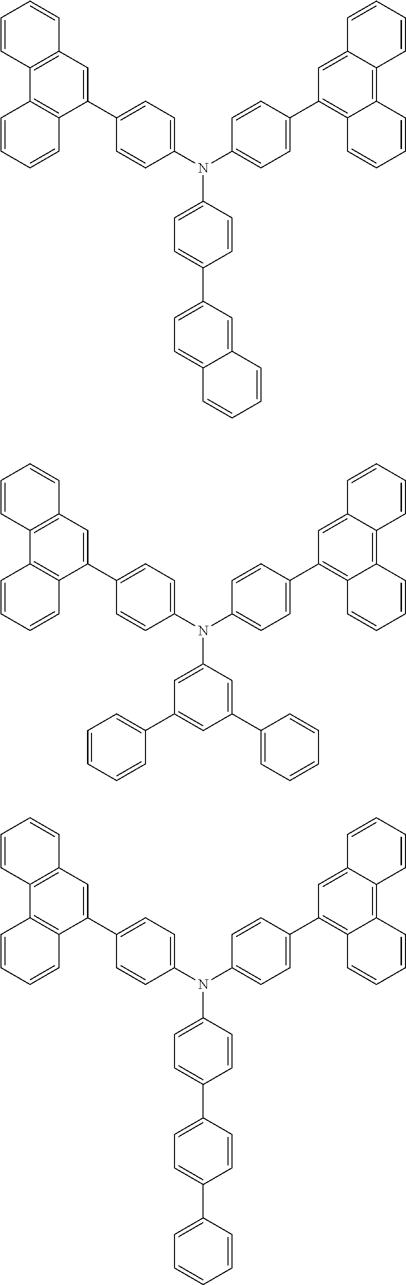 [SMILES: c1ccc2cc(-c3ccc(N(c4ccc(-c5cc6ccccc6c6ccccc56)cc4)c4ccc(-c5cc6ccccc6c6ccccc56)cc4)cc3)ccc2c1 ; c1ccc(-c2cc(-c3ccccc3)cc(N(c3ccc(-c4cc5ccccc5c5ccccc45)cc3)c3ccc(-c4cc5ccccc5c5ccccc45)cc3)c2)cc1 ; c1ccc(-c2ccc(-c3ccc(N(c4ccc(-c5cc6ccccc6c6ccccc56)cc4)c4ccc(-c5cc6ccccc6c6ccccc56)cc4)cc3)cc2)cc1]

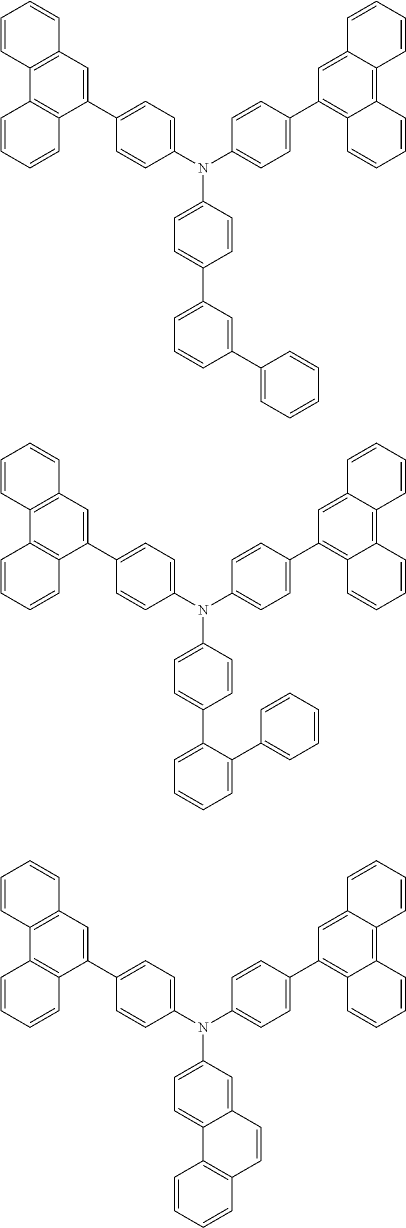 [SMILES: c1ccc(-c2cccc(-c3ccc(N(c4ccc(-c5cc6ccccc6c6ccccc56)cc4)c4ccc(-c5cc6ccccc6c6ccccc56)cc4)cc3)c2)cc1 ; c1ccc(-c2ccccc2-c2ccc(N(c3ccc(-c4cc5ccccc5c5ccccc45)cc3)c3ccc(-c4cc5ccccc5c5ccccc45)cc3)cc2)cc1 ; c1ccc2c(c1)ccc1cc(N(c3ccc(-c4cc5ccccc5c5ccccc45)cc3)c3ccc(-c4cc5ccccc5c5ccccc45)cc3)ccc12]

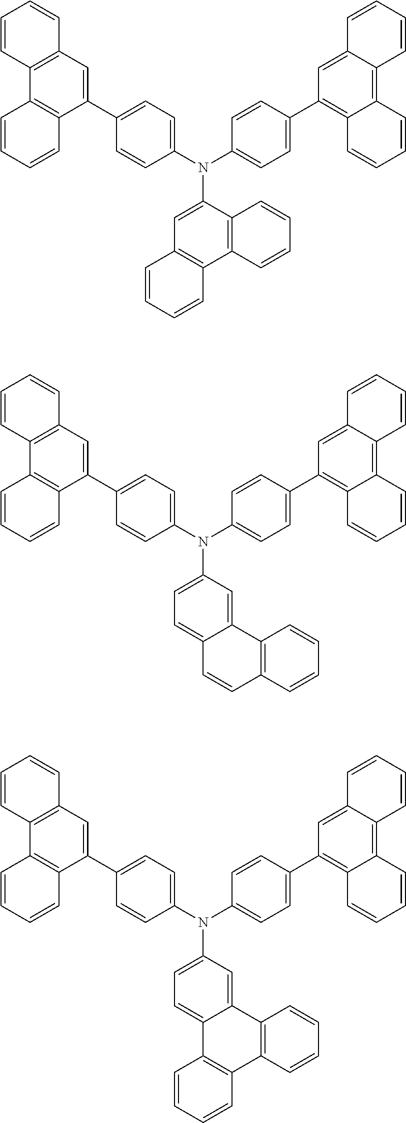 [SMILES: c1ccc2c(c1)cc(-c1ccc(N(c3ccc(-c4cc5ccccc5c5ccccc45)cc3)c3cc4ccccc4c4ccccc34)cc1)c1ccccc12 ; c1ccc2c(c1)ccc1ccc(N(c3ccc(-c4cc5ccccc5c5ccccc45)cc3)c3ccc(-c4cc5ccccc5c5ccccc45)cc3)cc12 ; c1ccc2c(c1)cc(-c1ccc(N(c3ccc(-c4cc5ccccc5c5ccccc45)cc3)c3ccc4c5ccccc5c5ccccc5c4c3)cc1)c1ccccc12]

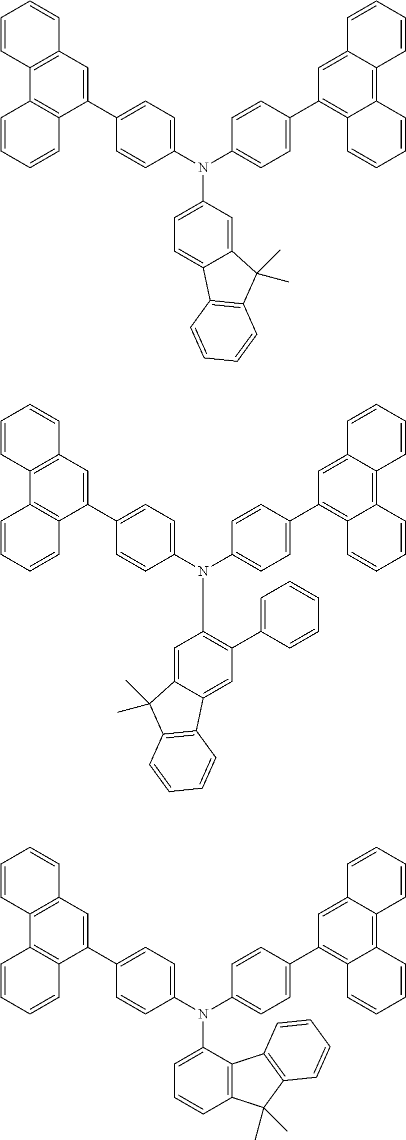 [SMILES: CC1(C)c2ccccc2-c2ccc(N(c3ccc(-c4cc5ccccc5c5ccccc45)cc3)c3ccc(-c4cc5ccccc5c5ccccc45)cc3)cc21 ; CC1(C)c2ccccc2-c2cc(-c3ccccc3)c(N(c3ccc(-c4cc5ccccc5c5ccccc45)cc3)c3ccc(-c4cc5ccccc5c5ccccc45)cc3)cc21 ; CC1(C)c2ccccc2-c2c(N(c3ccc(-c4cc5ccccc5c5ccccc45)cc3)c3ccc(-c4cc5ccccc5c5ccccc45)cc3)cccc21]

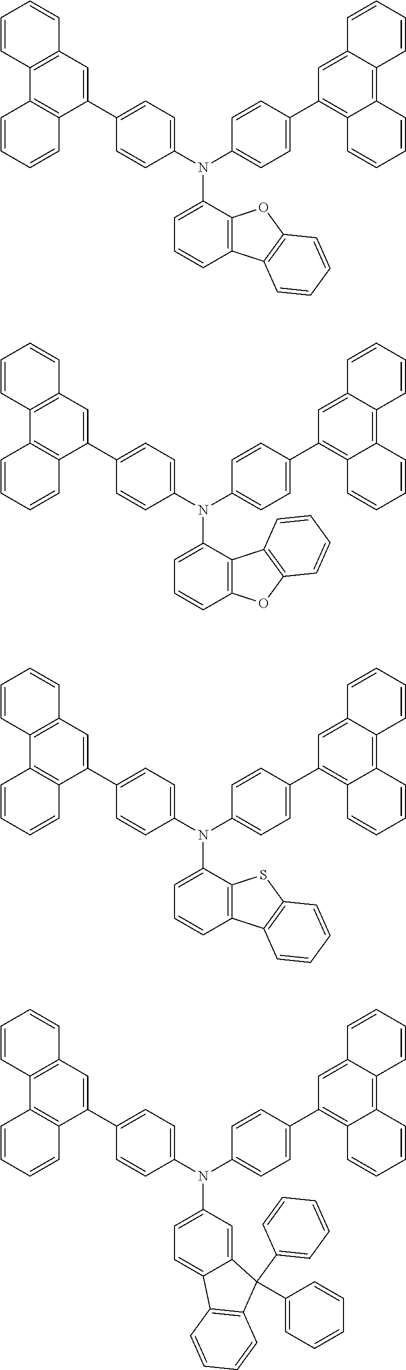 [SMILES: c1ccc2c(c1)cc(-c1ccc(N(c3ccc(-c4cc5ccccc5c5ccccc45)cc3)c3cccc4c3oc3ccccc34)cc1)c1ccccc12 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc2c(c1)cc(-c1ccc(N(c3ccc(-c4cc5ccccc5c5ccccc45)cc3)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4ccc(-c5cc6ccccc6c6ccccc56)cc4)cc32)cc1]

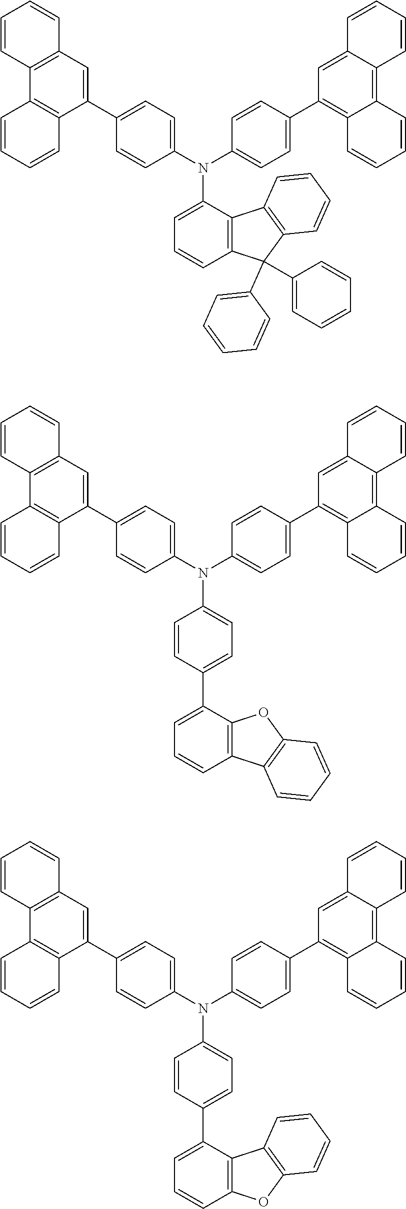 [SMILES: c1ccc(C2(c3ccccc3)c3ccccc3-c3c(N(c4ccc(-c5cc6ccccc6c6ccccc56)cc4)c4ccc(-c5cc6ccccc6c6ccccc56)cc4)cccc32)cc1 ; c1ccc2c(c1)cc(-c1ccc(N(c3ccc(-c4cc5ccccc5c5ccccc45)cc3)c3ccc(-c4cccc5c4oc4ccccc45)cc3)cc1)c1ccccc12 ; c1ccc2c(c1)cc(-c1ccc(N(c3ccc(-c4cc5ccccc5c5ccccc45)cc3)c3ccc(-c4cccc5oc6ccccc6c45)cc3)cc1)c1ccccc12]

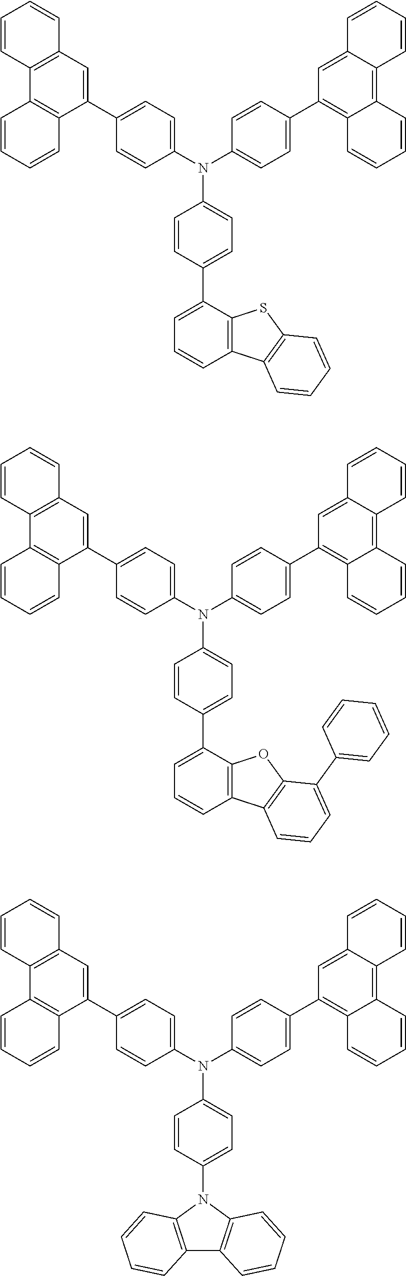 [SMILES: c1ccc2c(c1)cc(-c1ccc(N(c3ccc(-c4cc5ccccc5c5ccccc45)cc3)c3ccc(-c4cccc5c4sc4ccccc45)cc3)cc1)c1ccccc12 ; c1ccc(-c2cccc3c2oc2c(-c4ccc(N(c5ccc(-c6cc7ccccc7c7ccccc67)cc5)c5ccc(-c6cc7ccccc7c7ccccc67)cc5)cc4)cccc23)cc1 ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12]

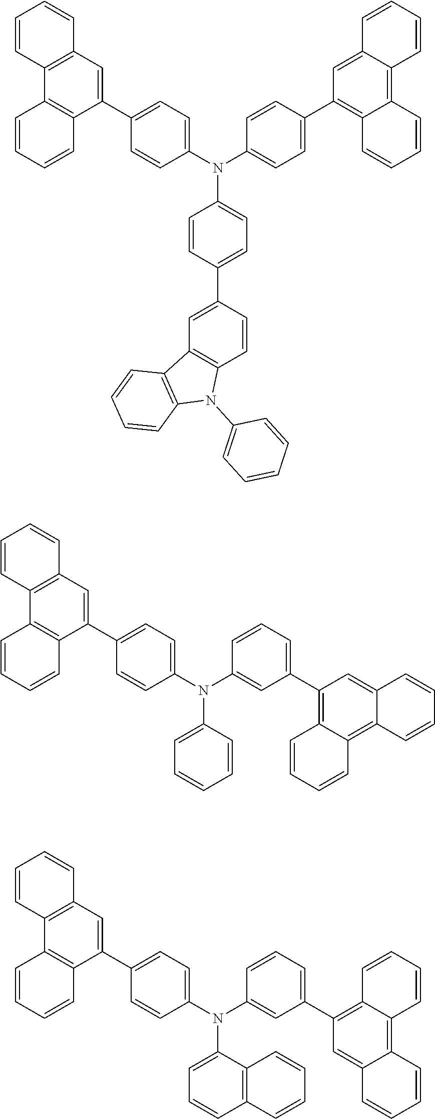 [SMILES: c1ccc(-n2c3ccccc3c3cc(-c4ccc(N(c5ccc(-c6cc7ccccc7c7ccccc67)cc5)c5ccc(-c6cc7ccccc7c7ccccc67)cc5)cc4)ccc32)cc1 ; c1ccc(N(c2ccc(-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3cc4ccccc4c4ccccc34)cc2)c2cccc3ccccc23)c1]

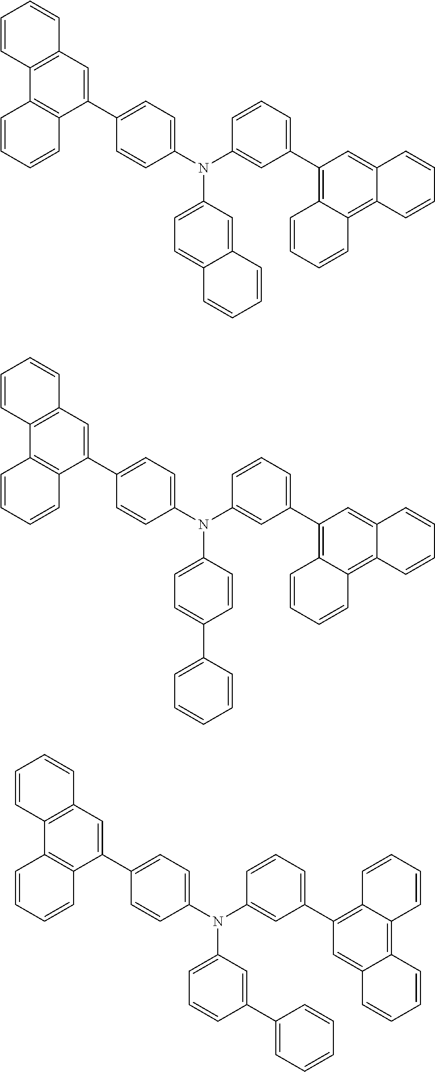 [SMILES: c1cc(-c2cc3ccccc3c3ccccc23)cc(N(c2ccc(-c3cc4ccccc4c4ccccc34)cc2)c2ccc3ccccc3c2)c1 ; c1ccc(-c2ccc(N(c3ccc(-c4cc5ccccc5c5ccccc45)cc3)c3cccc(-c4cc5ccccc5c5ccccc45)c3)cc2)cc1 ; c1ccc(-c2cccc(N(c3ccc(-c4cc5ccccc5c5ccccc45)cc3)c3cccc(-c4cc5ccccc5c5ccccc45)c3)c2)cc1]

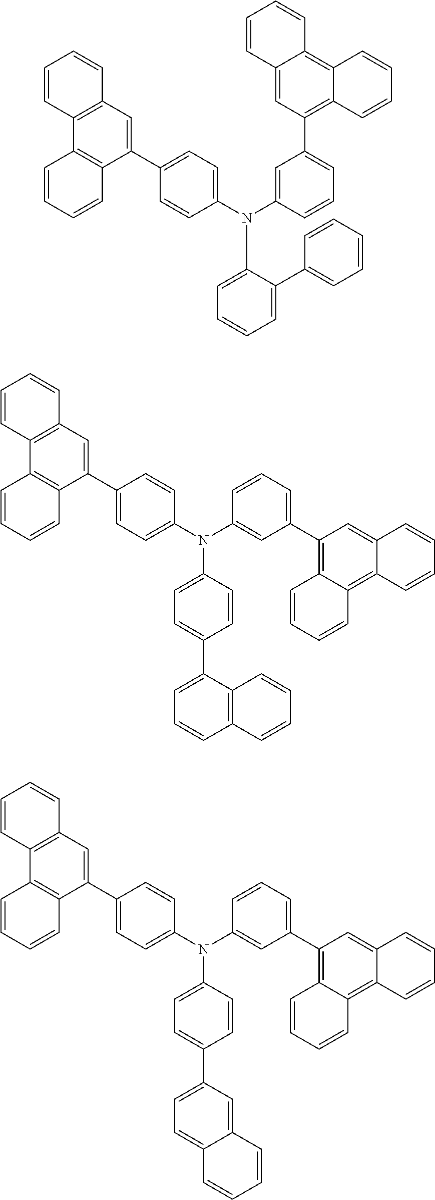 [SMILES: c1ccc(-c2ccccc2N(c2ccc(-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2ccc(-c3cc4ccccc4c4ccccc34)cc2)c1 ; c1cc(-c2cc3ccccc3c3ccccc23)cc(N(c2ccc(-c3ccc4ccccc4c3)cc2)c2ccc(-c3cc4ccccc4c4ccccc34)cc2)c1]

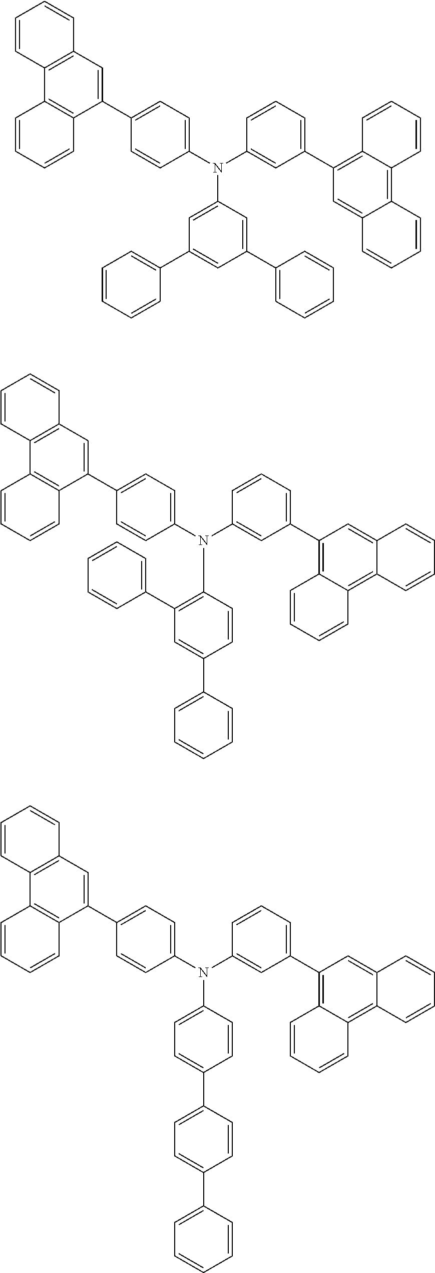 [SMILES: c1ccc(-c2cc(-c3ccccc3)cc(N(c3ccc(-c4cc5ccccc5c5ccccc45)cc3)c3cccc(-c4cc5ccccc5c5ccccc45)c3)c2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1 ; c1ccc(-c2ccc(-c3ccc(N(c4ccc(-c5cc6ccccc6c6ccccc56)cc4)c4cccc(-c5cc6ccccc6c6ccccc56)c4)cc3)cc2)cc1]

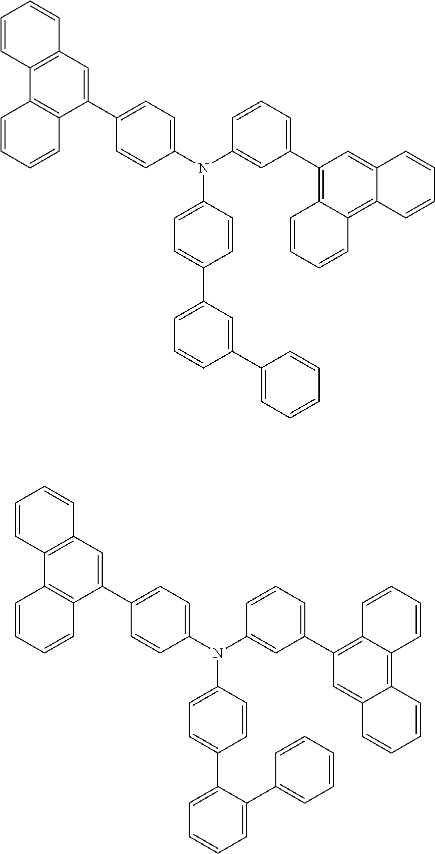 [SMILES: c1ccc(-c2cccc(-c3ccc(N(c4ccc(-c5cc6ccccc6c6ccccc56)cc4)c4cccc(-c5cc6ccccc6c6ccccc56)c4)cc3)c2)cc1 ; c1ccc(-c2ccccc2-c2ccc(N(c3ccc(-c4cc5ccccc5c5ccccc45)cc3)c3cccc(-c4cc5ccccc5c5ccccc45)c3)cc2)cc1]

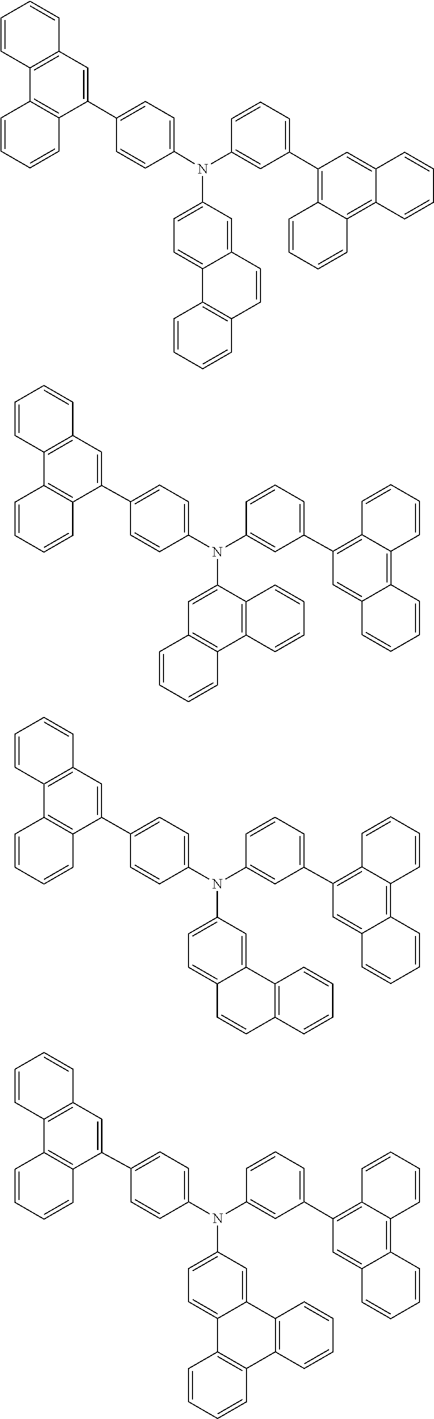 [SMILES: c1cc(-c2cc3ccccc3c3ccccc23)cc(N(c2ccc(-c3cc4ccccc4c4ccccc34)cc2)c2ccc3c(ccc4ccccc43)c2)c1 ; c1cc(-c2cc3ccccc3c3ccccc23)cc(N(c2ccc(-c3cc4ccccc4c4ccccc34)cc2)c2cc3ccccc3c3ccccc23)c1 ; c1cc(-c2cc3ccccc3c3ccccc23)cc(N(c2ccc(-c3cc4ccccc4c4ccccc34)cc2)c2ccc3ccc4ccccc4c3c2)c1 ; c1cc(-c2cc3ccccc3c3ccccc23)cc(N(c2ccc(-c3cc4ccccc4c4ccccc34)cc2)c2ccc3c4ccccc4c4ccccc4c3c2)c1]

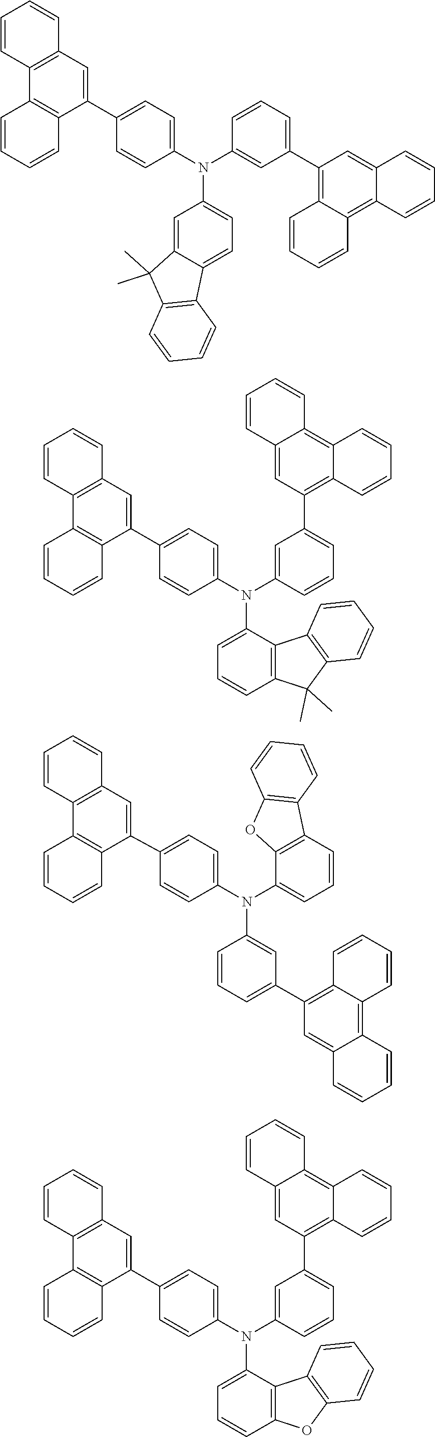

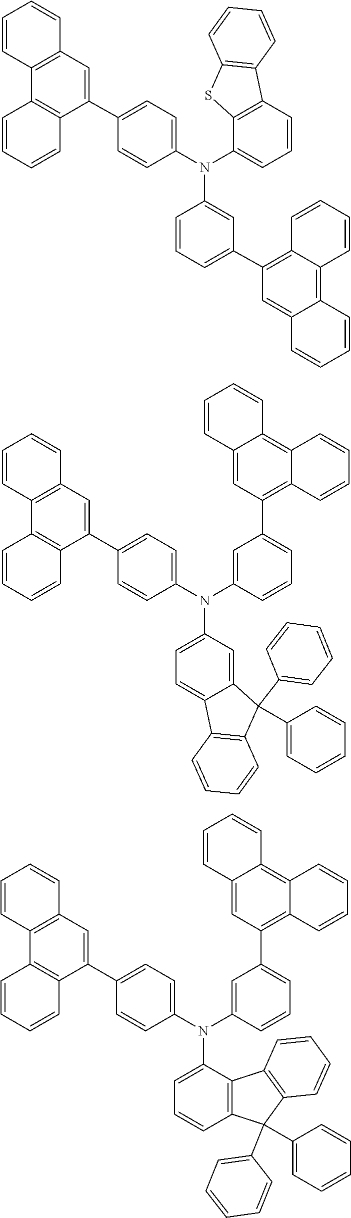

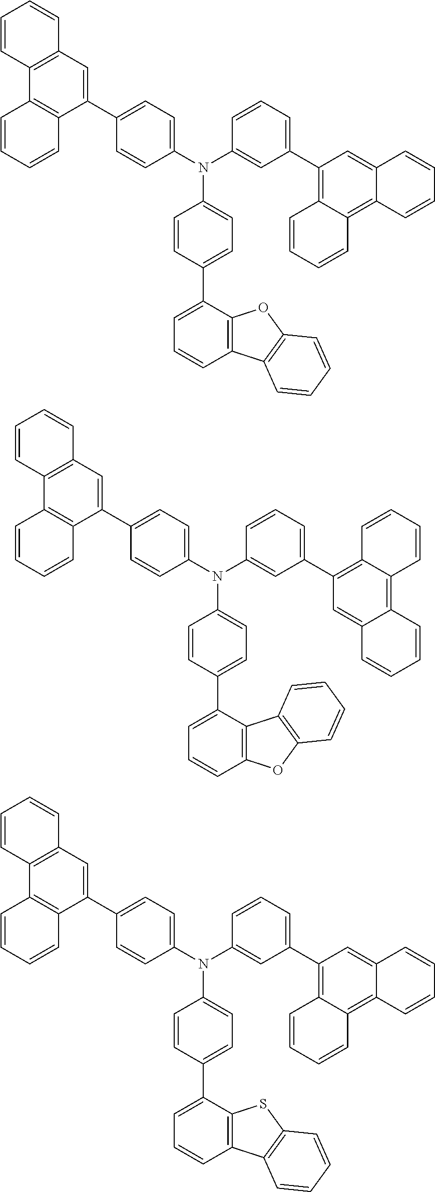 [SMILES: c1cc(-c2cc3ccccc3c3ccccc23)cc(N(c2ccc(-c3cc4ccccc4c4ccccc34)cc2)c2ccc(-c3cccc4c3oc3ccccc34)cc2)c1 ; c1cc(-c2cc3ccccc3c3ccccc23)cc(N(c2ccc(-c3cc4ccccc4c4ccccc34)cc2)c2ccc(-c3cccc4oc5ccccc5c34)cc2)c1 ; c1cc(-c2cc3ccccc3c3ccccc23)cc(N(c2ccc(-c3cc4ccccc4c4ccccc34)cc2)c2ccc(-c3cccc4c3sc3ccccc34)cc2)c1]

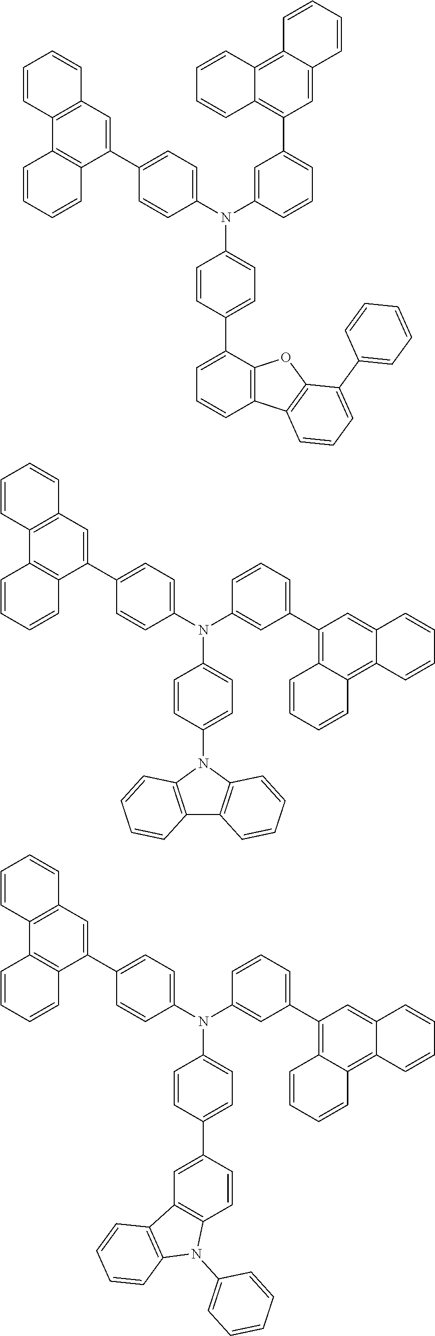 [SMILES: c1ccc(-c2cccc3c2oc2c(-c4ccc(N(c5ccc(-c6cc7ccccc7c7ccccc67)cc5)c5cccc(-c6cc7ccccc7c7ccccc67)c5)cc4)cccc23)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1 ; c1ccc(-n2c3ccccc3c3cc(-c4ccc(N(c5ccc(-c6cc7ccccc7c7ccccc67)cc5)c5cccc(-c6cc7ccccc7c7ccccc67)c5)cc4)ccc32)cc1]

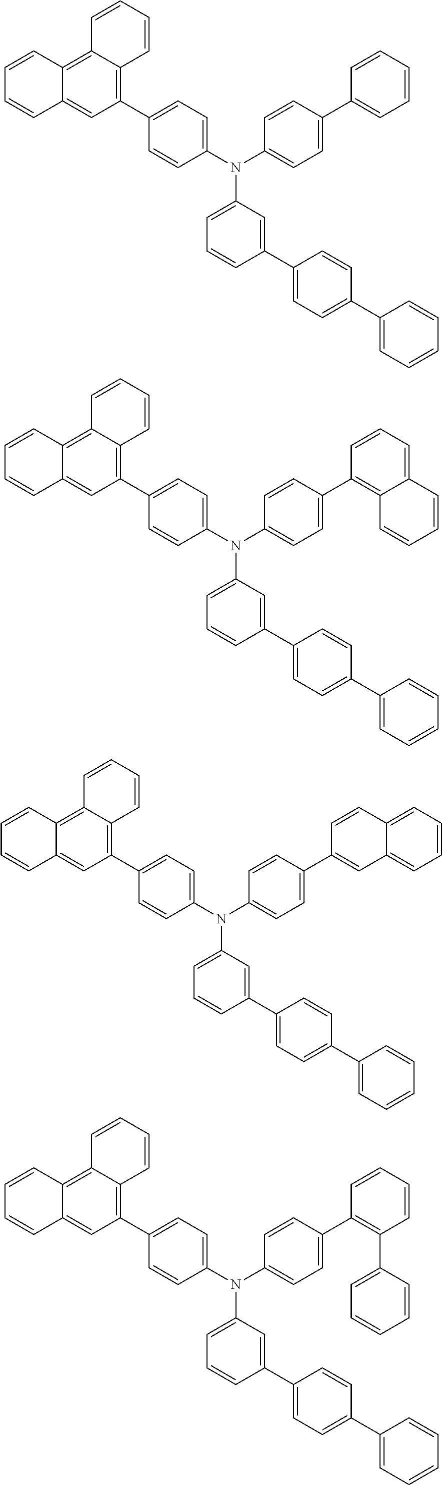 [SMILES: c1ccc(-c2ccc(-c3cccc(N(c4ccc(-c5ccccc5)cc4)c4ccc(-c5cc6ccccc6c6ccccc56)cc4)c3)cc2)cc1 ; c1ccc(-c2ccc(-c3cccc(N(c4ccc(-c5cccc6ccccc56)cc4)c4ccc(-c5cc6ccccc6c6ccccc56)cc4)c3)cc2)cc1 ; c1ccc(-c2ccc(-c3cccc(N(c4ccc(-c5ccc6ccccc6c5)cc4)c4ccc(-c5cc6ccccc6c6ccccc56)cc4)c3)cc2)cc1 ; c1ccc(-c2ccc(-c3cccc(N(c4ccc(-c5ccccc5-c5ccccc5)cc4)c4ccc(-c5cc6ccccc6c6ccccc56)cc4)c3)cc2)cc1]

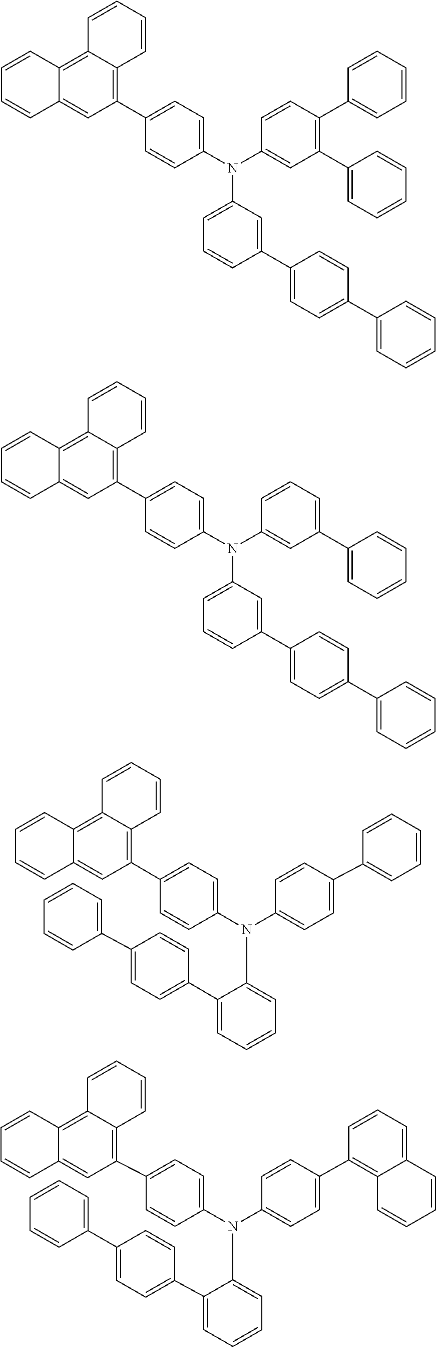 [SMILES: c1ccc(-c2ccc(-c3cccc(N(c4ccc(-c5cc6ccccc6c6ccccc56)cc4)c4ccc(-c5ccccc5)c(-c5ccccc5)c4)c3)cc2)cc1 ; c1ccc(-c2ccc(-c3cccc(N(c4ccc(-c5cc6ccccc6c6ccccc56)cc4)c4cccc(-c5ccccc5)c4)c3)cc2)cc1 ; c1ccc(-c2ccc(-c3ccccc3N(c3ccc(-c4ccccc4)cc3)c3ccc(-c4cc5ccccc5c5ccccc45)cc3)cc2)cc1 ; c1ccc(-c2ccc(-c3ccccc3N(c3ccc(-c4cccc5ccccc45)cc3)c3ccc(-c4cc5ccccc5c5ccccc45)cc3)cc2)cc1]

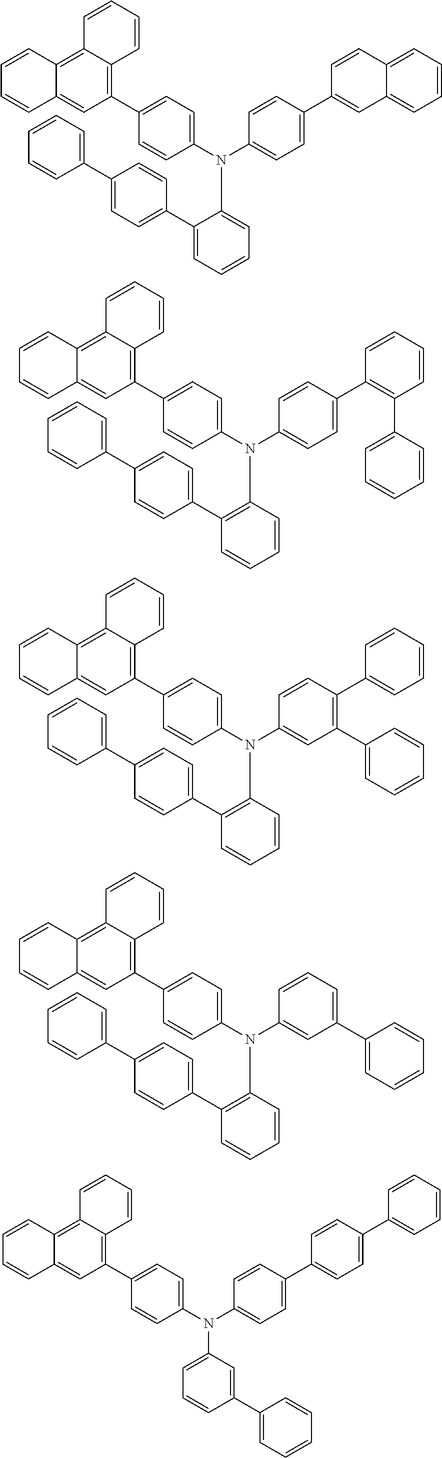 [SMILES: c1ccc(-c2ccc(-c3ccccc3N(c3ccc(-c4ccc5ccccc5c4)cc3)c3ccc(-c4cc5ccccc5c5ccccc45)cc3)cc2)cc1 ; c1ccc(-c2ccc(-c3ccccc3N(c3ccc(-c4ccccc4-c4ccccc4)cc3)c3ccc(-c4cc5ccccc5c5ccccc45)cc3)cc2)cc1 ; c1ccc(-c2ccc(-c3ccccc3N(c3ccc(-c4cc5ccccc5c5ccccc45)cc3)c3ccc(-c4ccccc4)c(-c4ccccc4)c3)cc2)cc1 ; c1ccc(-c2ccc(-c3ccccc3N(c3ccc(-c4cc5ccccc5c5ccccc45)cc3)c3cccc(-c4ccccc4)c3)cc2)cc1 ; c1ccc(-c2ccc(-c3ccc(N(c4ccc(-c5cc6ccccc6c6ccccc56)cc4)c4cccc(-c5ccccc5)c4)cc3)cc2)cc1]

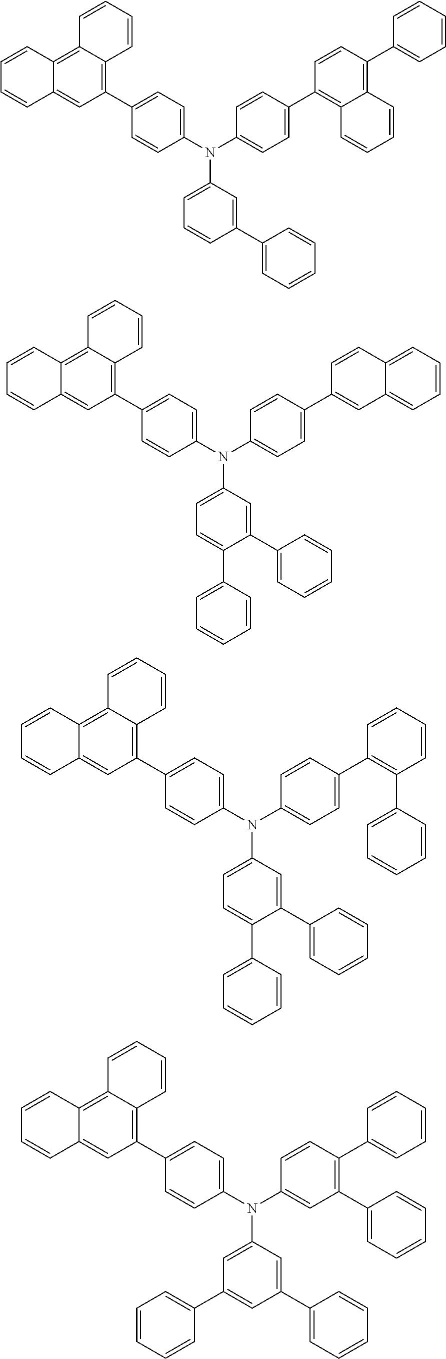 [SMILES: c1ccc(-c2cccc(N(c3ccc(-c4ccc(-c5ccccc5)c5ccccc45)cc3)c3ccc(-c4cc5ccccc5c5ccccc45)cc3)c2)cc1 ; c1ccc(-c2ccc(N(c3ccc(-c4ccc5ccccc5c4)cc3)c3ccc(-c4cc5ccccc5c5ccccc45)cc3)cc2-c2ccccc2)cc1 ; c1ccc(-c2ccccc2-c2ccc(N(c3ccc(-c4cc5ccccc5c5ccccc45)cc3)c3ccc(-c4ccccc4)c(-c4ccccc4)c3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1]

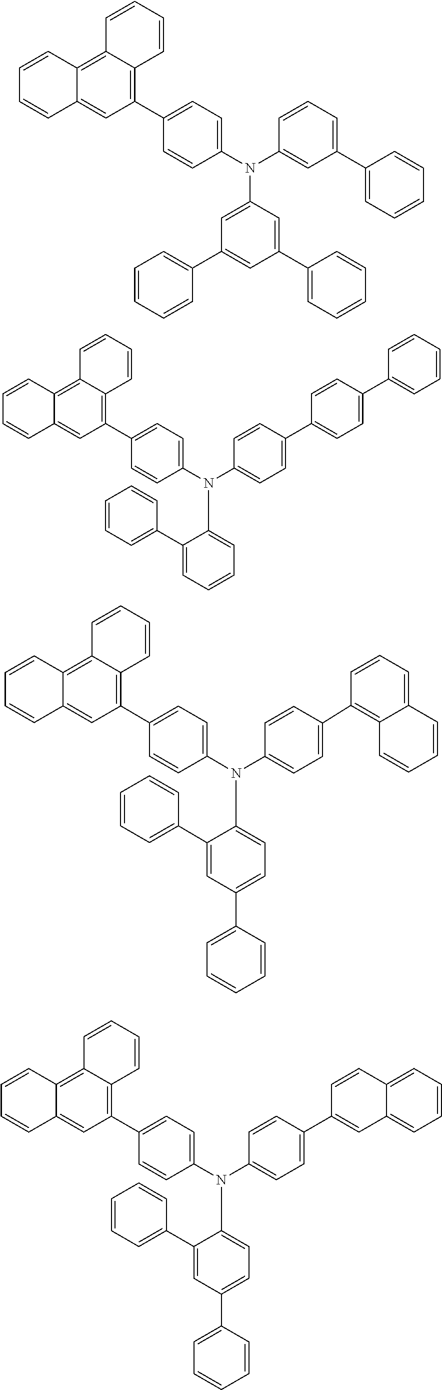

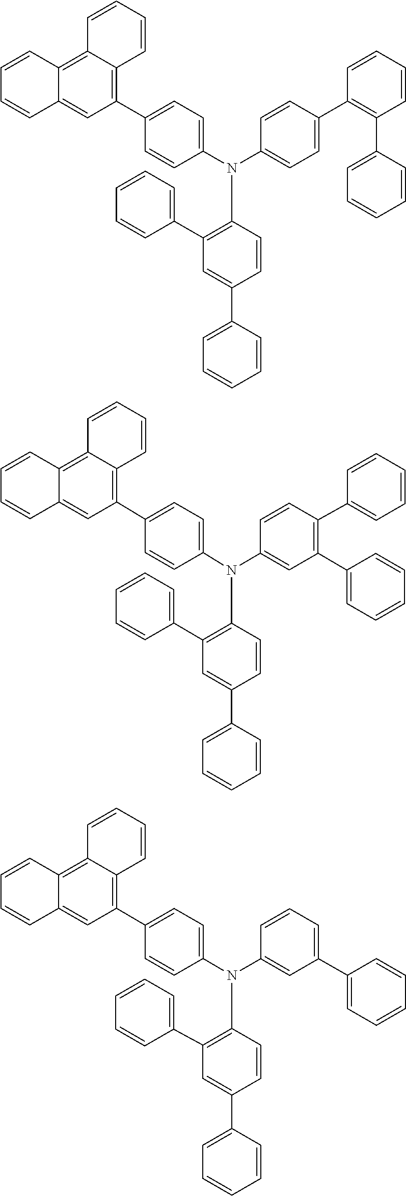 [SMILES: c1ccc(-c2ccc(N(c3ccc(-c4ccccc4-c4ccccc4)cc3)c3ccc(-c4cc5ccccc5c5ccccc45)cc3)c(-c3ccccc3)c2)cc1 ; c1ccc(-c2ccc(N(c3ccc(-c4cc5ccccc5c5ccccc45)cc3)c3ccc(-c4ccccc4)c(-c4ccccc4)c3)c(-c3ccccc3)c2)cc1 ; c1ccc(-c2cccc(N(c3ccc(-c4cc5ccccc5c5ccccc45)cc3)c3ccc(-c4ccccc4)cc3-c3ccccc3)c2)cc1]

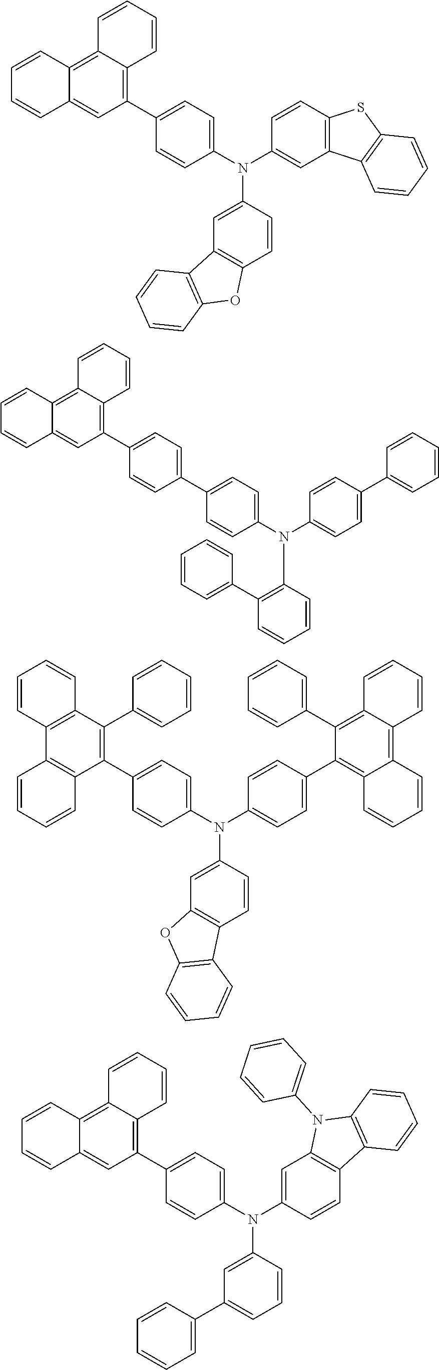

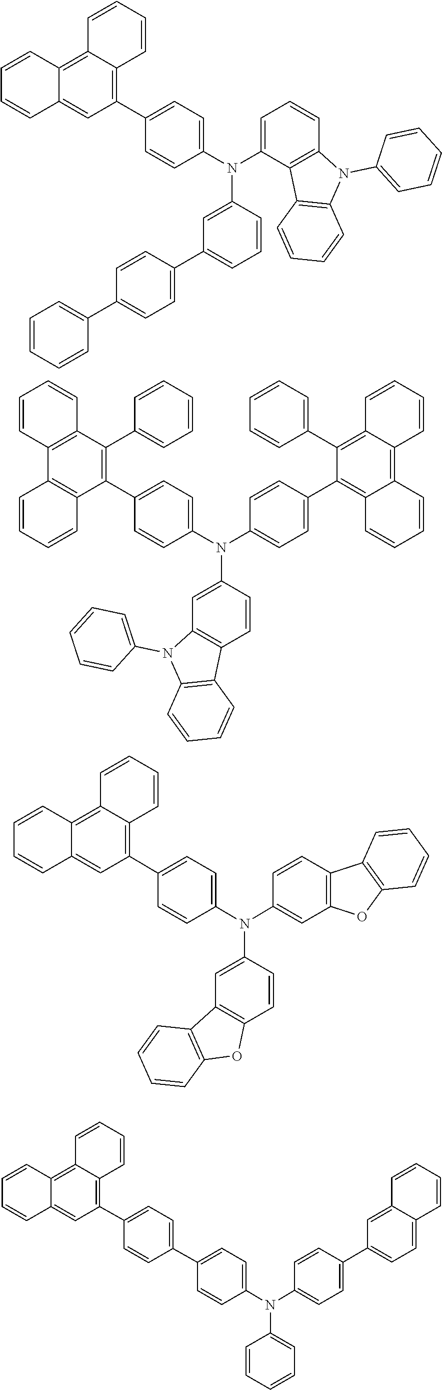

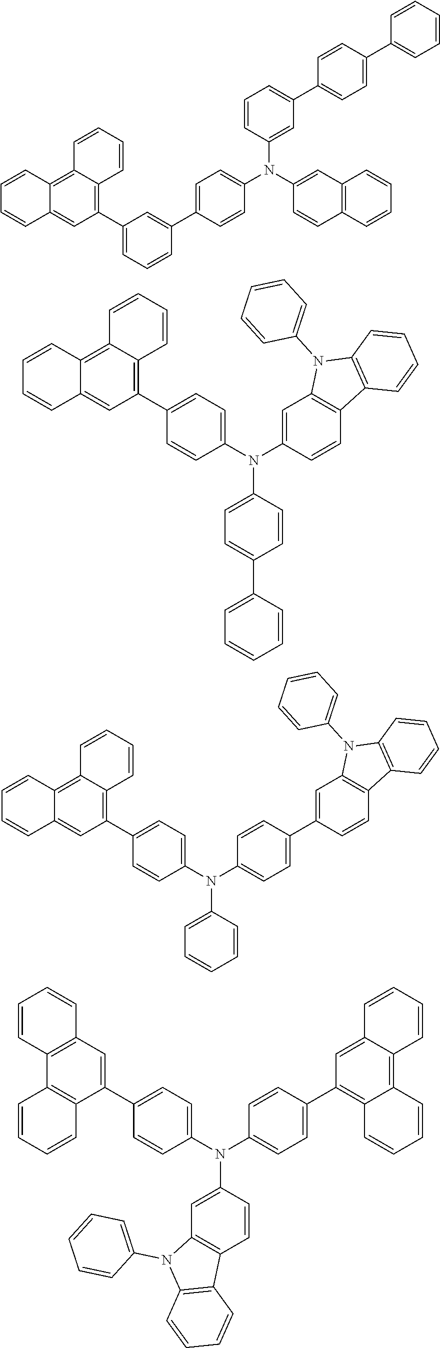

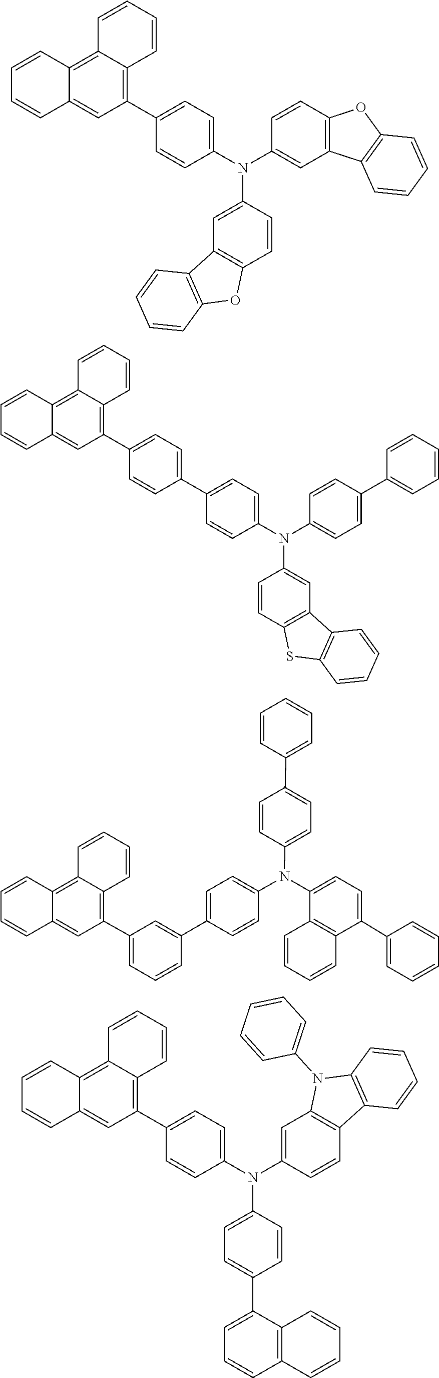

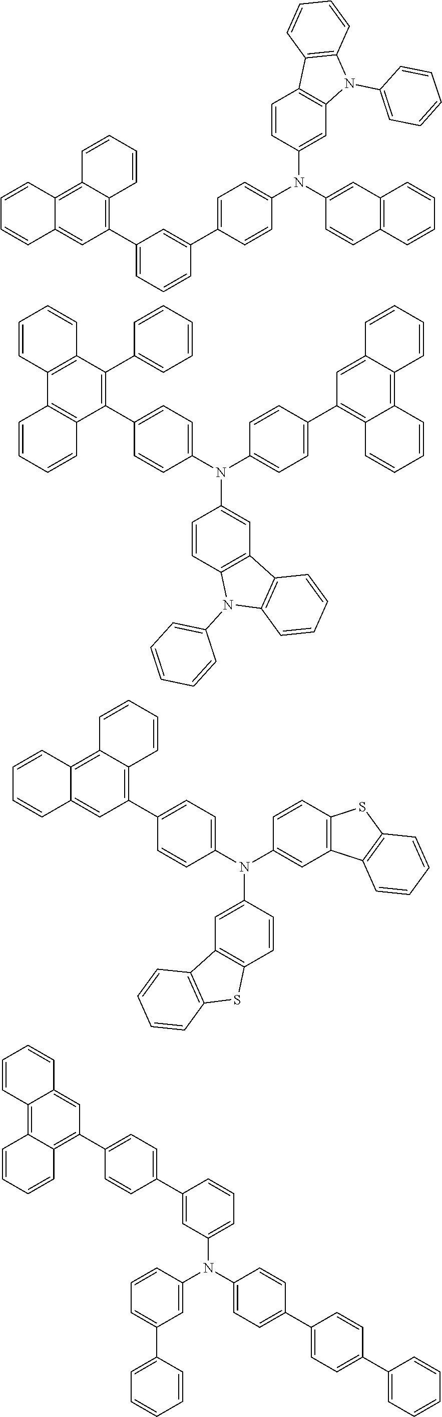

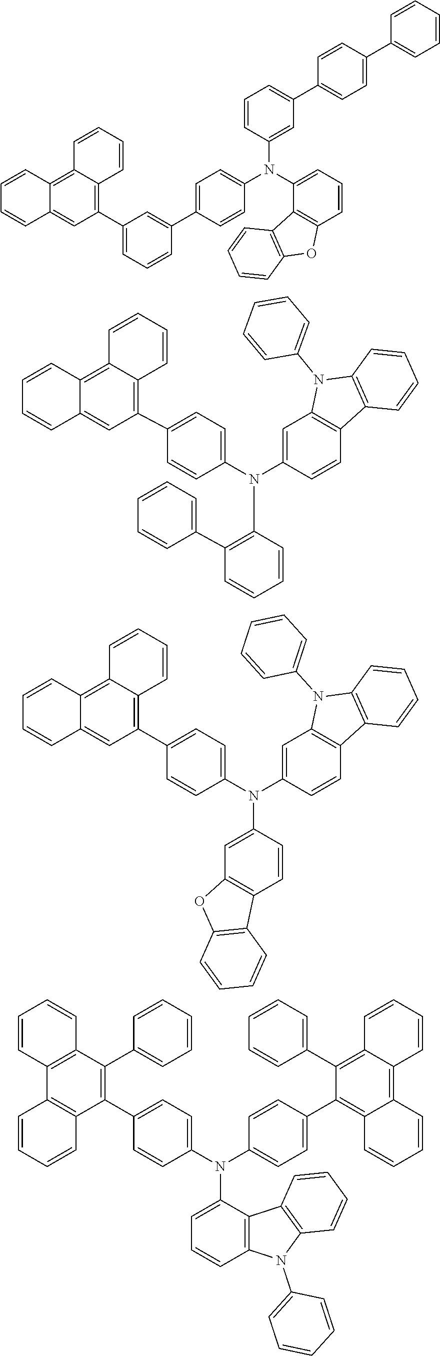

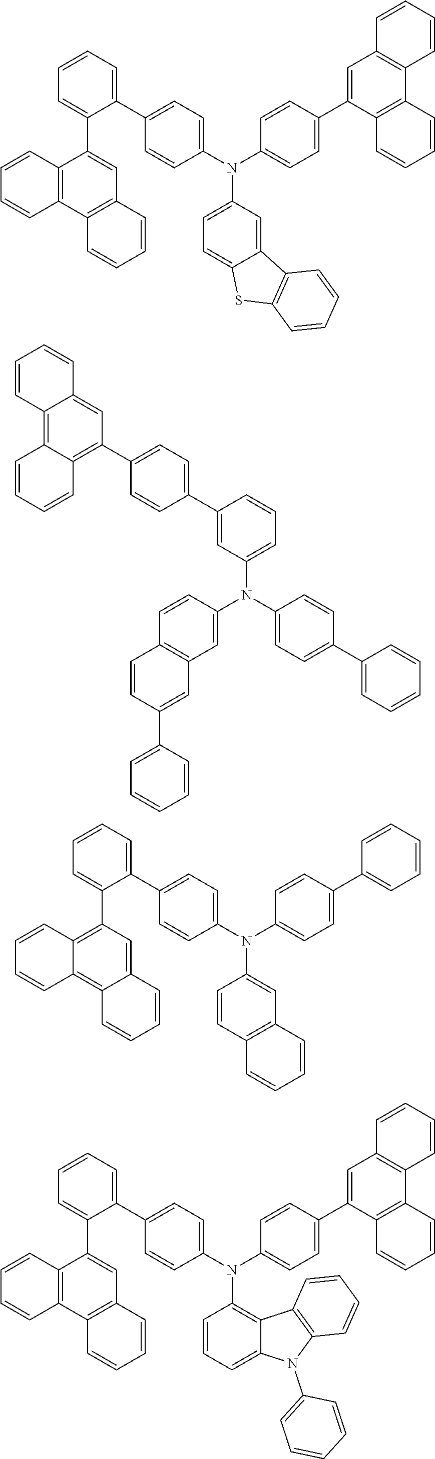

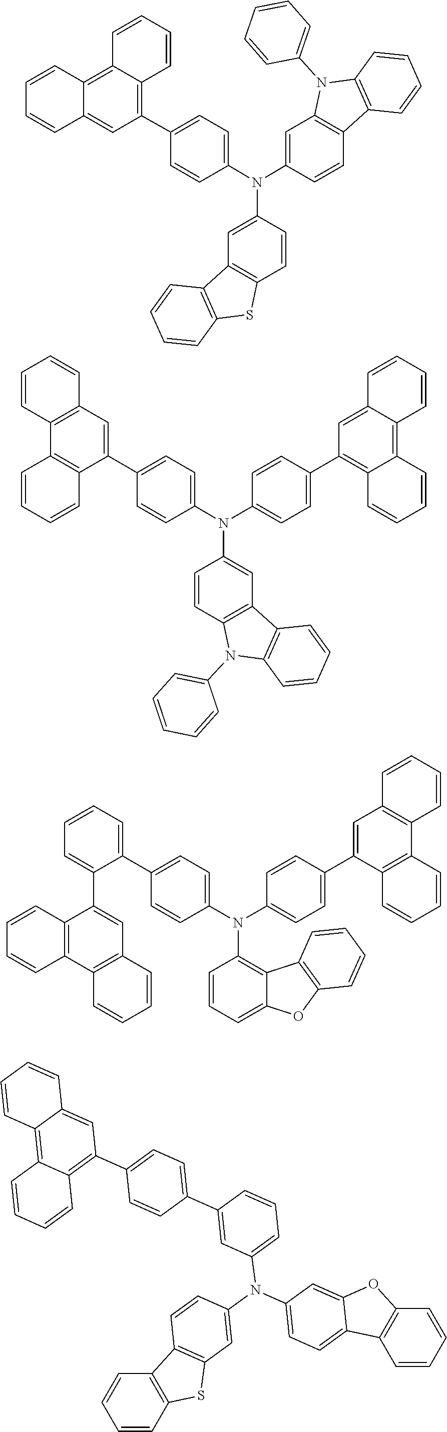 [SMILES: c1ccc(-n2c3ccccc3c3ccc(N(c4ccc(-c5cc6ccccc6c6ccccc56)cc4)c4ccc5sc6ccccc6c5c4)cc32)cc1 ; c1ccc(-n2c3ccccc3c3cc(N(c4ccc(-c5cc6ccccc6c6ccccc56)cc4)c4ccc(-c5cc6ccccc6c6ccccc56)cc4)ccc32)cc1 ; c1ccc(-c2cc3ccccc3c3ccccc23)c(-c2ccc(N(c3ccc(-c4cc5ccccc5c5ccccc45)cc3)c3cccc4oc5ccccc5c34)cc2)c1 ; c1cc(-c2ccc(-c3cc4ccccc4c4ccccc34)cc2)cc(N(c2ccc3c(c2)oc2ccccc23)c2ccc3c(c2)sc2ccccc23)c1]

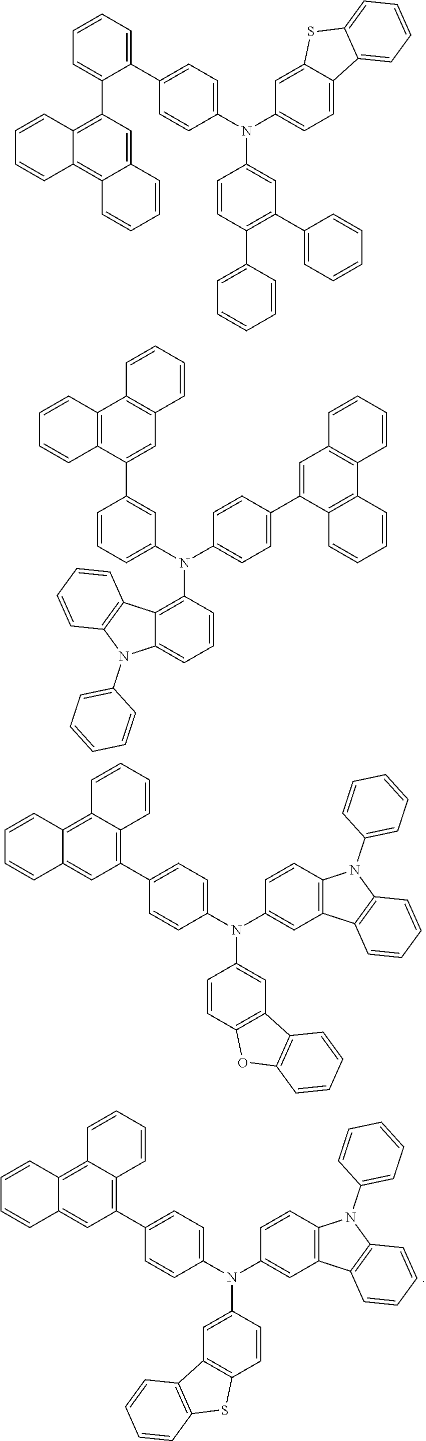

Meanwhile, the compound of Chemical Formula 2 can be prepared by, for example, a preparation method as shown in Reaction Scheme 2 below, and other compounds can be prepared similarly.

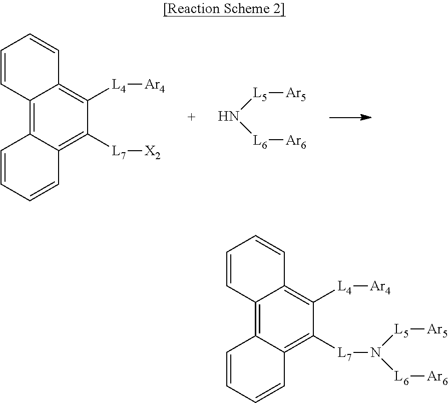

In Reaction Scheme 2, Ar₄ to Ar₆ and L₄ to L₇ are as defined in Chemical Formula 2, and X₂ is a halogen. Preferably, X₂ is chlorine or bromine. More preferably, X₂ is chlorine.

In addition, according to another embodiment of the present disclosure, Reaction Scheme 2 can further include an additional step of producing an amine compound, as shown in Reaction Scheme 2-1 below.

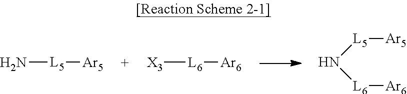

In Reaction Scheme 2-1, Ar₅ to Ar₆ and L₅ to L₆ are as defined in Chemical Formula 2, and X₃ is a halogen. Preferably, X₃ is chlorine or bromine. More preferably, X₃ is chlorine.

In addition, according to another embodiment of the present disclosure, Reaction Scheme 2 can further include an additional step of producing a phenanthrene-based compound, as shown in Reaction Scheme 2-2 below.

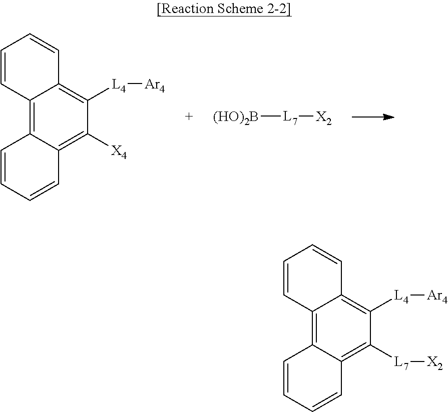

In Reaction Scheme 2-2, Ar₄, L₄, and L₇ are as defined in Chemical Formula 2, and X₂ and X₄ are each independently a halogen. Preferably, X₂ and X₄ are each independently chlorine or bromine. More preferably, X₂ and X₄ are different halogens, where X₂ is chlorine and X₄ is bromine.

In the present disclosure, the compound of Chemical Formula 2 can be prepared by performing Reaction Formula 2-1 and Reaction Formula 2-2 separately and then performing Reaction Formula 2. Alternatively, depending on the type of substituent, the compound of Chemical Formula 2 can be prepared by performing Reaction Formula 2-1 and Reaction Formula 2 in a batch.

Specifically, Reaction Schemes 2 and 2-1 are performed as amine substitution reactions. The amine substitution reaction is preferably performed in the presence of a palladium catalyst and a base, respectively, and the reactive group for the amine substitution reaction can be appropriately changed. The above preparation method can be further embodied in the Preparation Examples described hereinafter.

In addition, the Reaction Scheme 2-2 is performed as a Suzuki-coupling reaction. The Suzuki-coupling reaction is preferably performed in the presence of a palladium catalyst and a base, respectively, and the reactive group for the Suzuki-coupling reaction can be appropriately changed. The above preparation method can be further embodied in the Preparation Examples described hereinafter.

For example, in Reaction Schemes 2 and 2-1 to 2-2, sodium tert-butoxide (NaOtBu), potassium carbonate (K₂CO₃), sodium bicarbonate (NaHCO₃), cesium carbonate (Cs₂CO₃), sodium acetate (NaOAc), potassium acetate (KOAc), sodium ethoxide (NaOEt), triethylamine (Et₃N), N,N-diisopropylethylamine (EtN(iPr)₂), or the like can be used as the base component. Preferably, the base component can be sodium tert-butoxide (NaOtBu), potassium carbonate (K₂CO₃), cesium carbonate (Cs₂CO₃), potassium acetate (KOAc), or N,N-diisopropylethylamine (EtN(iPr)₂). In particular, sodium tert-butoxide (NaOtBu) can be used as the base component in the Reaction Schemes 2 and 2-1, and potassium carbonate (K₂CO₃) can be used as the base component in Reaction Scheme 2-2.

Further, in Reaction Schemes 2 and 2-1 to 2-2, bis(tri-(tert-butyl)-phosphine)palladium (0) (Pd(P-tBu₃)₂), tetrakis(triphenylphosphine)-palladium (0) (Pd(PPh₃)₄), tris(dibenzylideneacetone)-dipalladium (0) (Pd₂(dba)₃), bis(dibenzylideneacetone)palladium (0) (Pd(dba)₂), palladium (II) acetate (Pd(OAc)₂), or the like can be used as the palladium catalyst. Preferably, the palladium catalyst can be bis(tri-(tert-butyl)phosphine)palladium (0) (Pd(P-tBu₃)₂), tetrakis(triphenylphosphine)-palladium (0) (Pd(PPh₃)₄), or bis(dibenzylideneacetone)palladium (0) (Pd(dba)₂). In particular, in Reaction Scheme 2, tetrakis(triphenylphosphine)palladium (0) (Pd(PPh₃)₄) can be used as the palladium catalyst. Specifically, bis(tri-(tert-butyl)phosphine)palladium (0) (Pd(P-tBu₃)₂) can be preferably used as the palladium catalyst in the Reaction Schemes 2 and 2-1, and tetrakis(triphenylphosphine)palladium (0) can be preferably used as the palladium catalyst in Reaction Scheme 2-2.

In the present disclosure, the first compound and the second compound can be included in the light emitting layer at a weight ratio of 1:99 to 99:1. For example, a weight ratio of the first compound and the second compound in the light emitting layer can be 5:95 to 95:5, or 10:90 to 90:10, or 20:80 to 80:20, or 30:70 to 70:30, or 40:60 to 60:40, or 50:50.

In addition, the light emitting layer can further include a dopant material.

Specifically, the organic light emitting device can include the compound of Chemical Formula 1, the compound of Chemical Formula 2, and a dopant material.

For example, the organic light emitting device can include the compound of Chemical Formula 1, the compound of Chemical Formula 2, and a dopant material at a weight ratio of 100:1 to 1:1, namely, the total contents of the compounds of Chemical Formula 1 and Chemical Formula 2: the content of the dopant.

Specifically, the organic light emitting device can include the compound of Chemical Formula 1, the compound of Chemical Formula 2, and a dopant material at a weight ratio of 100:1 to 2:1, namely, the total contents of the compounds of Chemical Formula 1 and Chemical Formula 2: the content of the dopant. For example, a weight ratio of the total contents of the compounds of Chemical Formula 1 and Chemical Formula 2: the content of the dopant can be 90:1 to 3:1 or 80:1 to 4:1, or 60:1 to 5:1.

The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

For example, the dopant material can be a metal complex.

Specifically, the dopant material can be an iridium complex.

In addition, the organic material layer can include a light emitting layer, the light emitting layer can include a dopant material, the dopant material can be selected from the group consisting of the following:

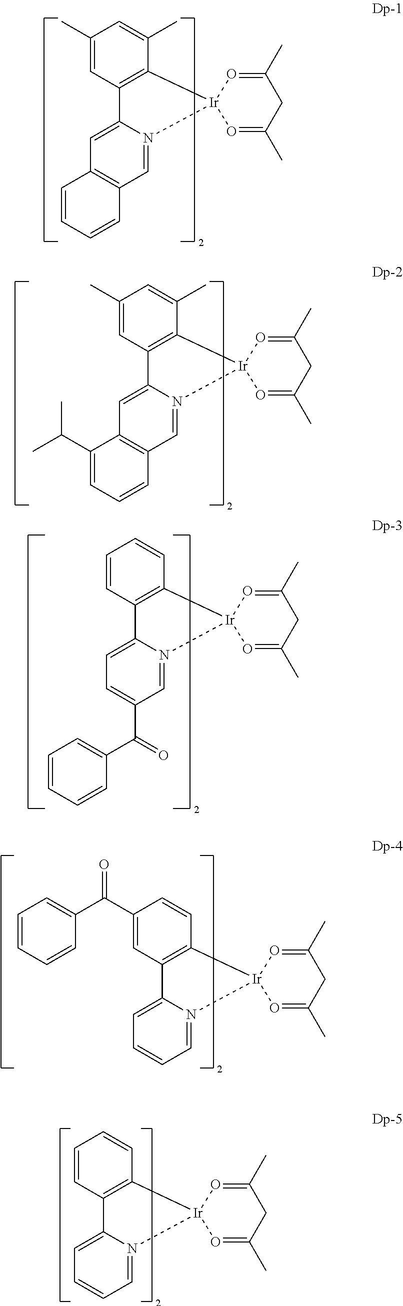

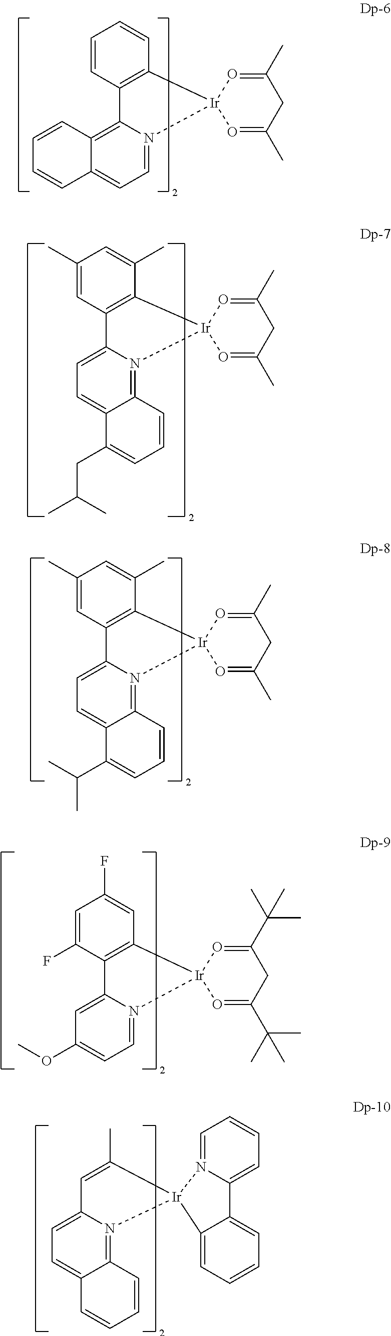

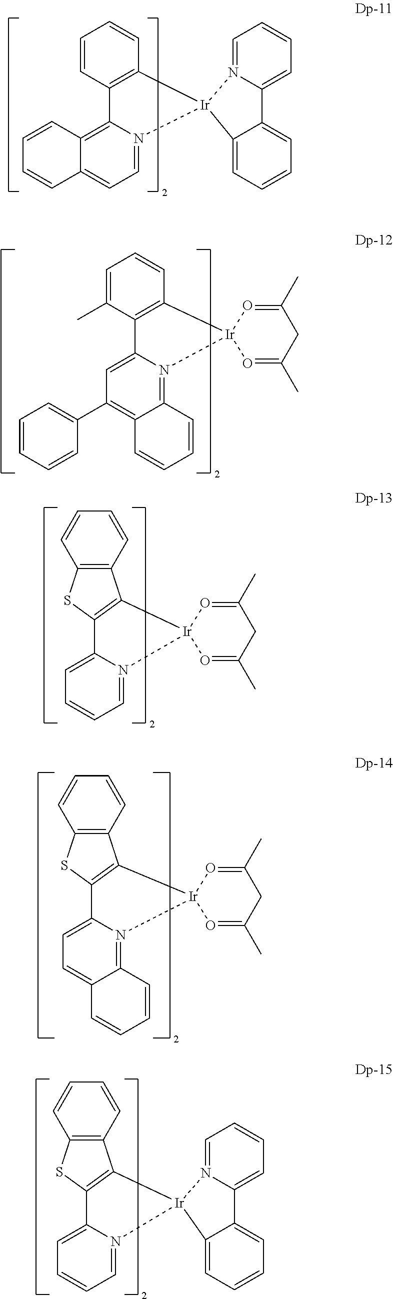

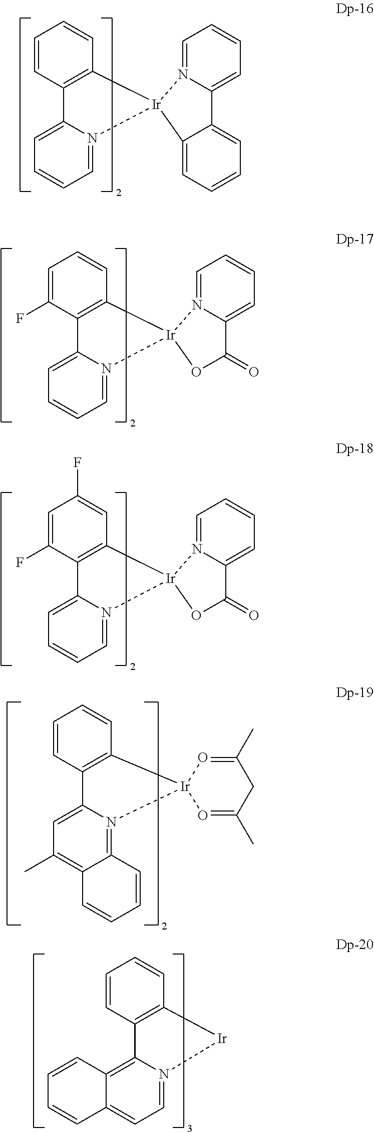

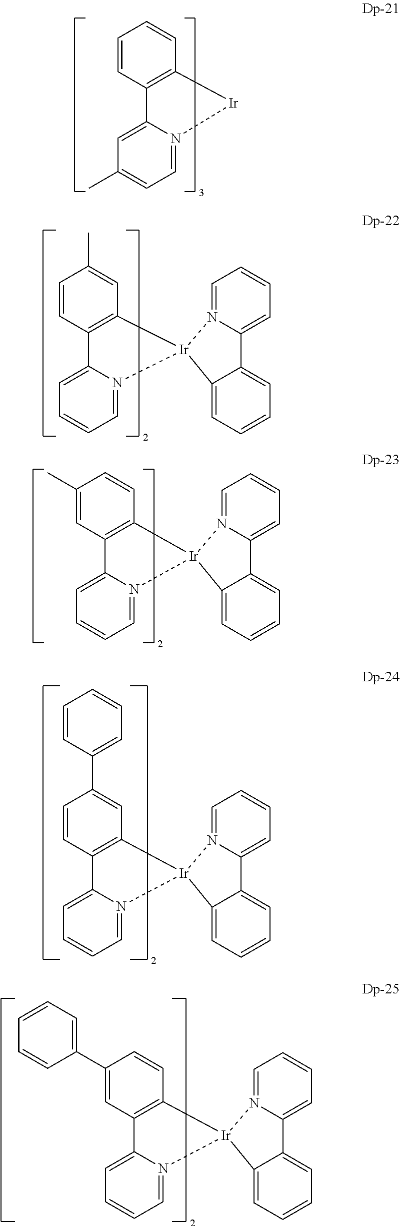

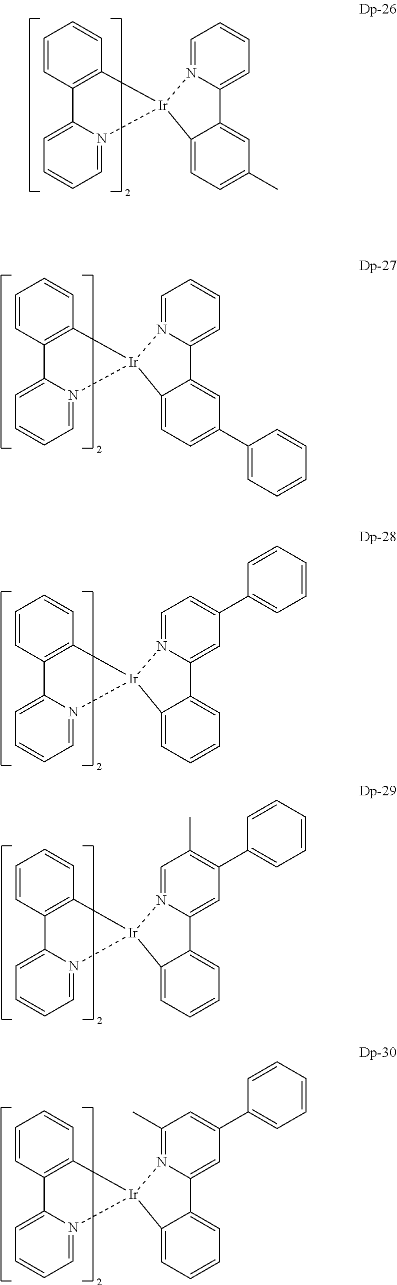

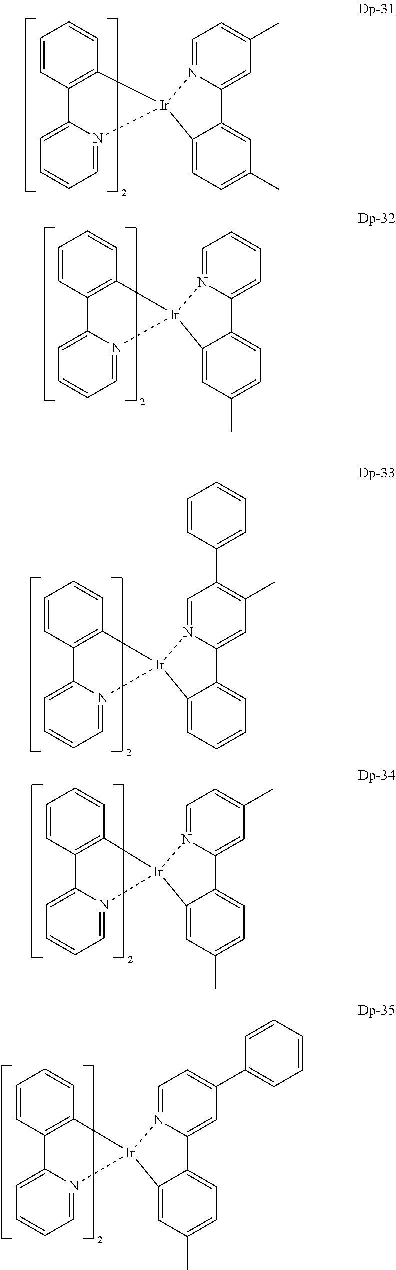

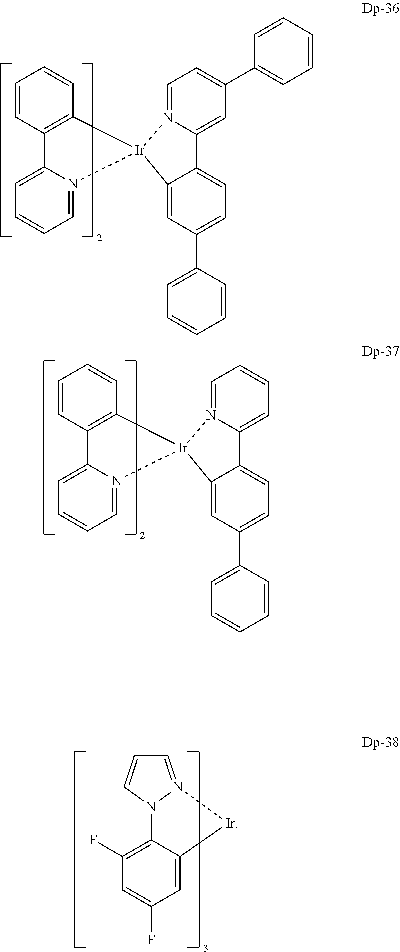

The dopant material can be one of the structures described above, but are not limited thereto.

### Hole Blocking Layer

The hole blocking layer is a layer provided between the electron transport layer and the light emitting layer in order to prevent the holes injected in the anode from being transferred to the electron transport layer without being recombined in the light emitting layer, which can also be referred to as a hole inhibition layer or a hole stopping layer. The hole blocking layer is preferably a material having the large ionization energy.

### Electron Transport Layer

The organic light emitting device according to the present disclosure can include an electron transport layer on the light emitting layer, if necessary.

The electron transport layer is a layer that receives the electrons from the cathode or the electron injection layer formed on the cathode and anode and transports the electrons to the light emitting layer, and that suppress the transfer of holes from the light emitting layer, and an electron transport material is suitably a material which can receive electrons well from a cathode and transfer the electrons to a light emitting layer, and has a large mobility for electrons.

Specific examples of the electron transport material include: an Al complex of 8-hydroxyquinoline; a complex including Alq₃; an organic radical compound; a hydroxyflavone-metal complex, and the like, but are not limited thereto. The electron transport layer can be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

### Electron Injection Layer

The organic light emitting device according to the present disclosure can further include an electron injection layer on the light emitting layer (or on an electron transport layer when the electron transport layer is present), if necessary.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the materials that can be used as the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron injection and transport layer” is a layer that performs both the roles of the electron injection layer and the electron transport layer, and the material that serves as each layer can be used alone or in combination, but is not limited thereto.

### Organic Light Emitting Device

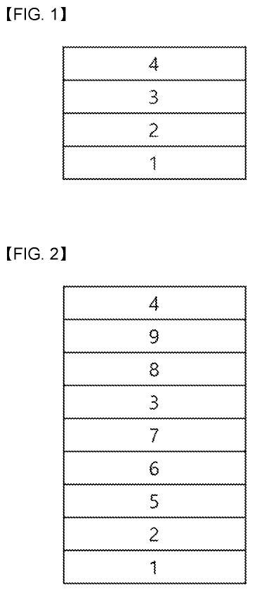

The structure of the organic light emitting device according to the present disclosure is illustrated in FIGS. 1 and 2. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by sequentially laminating the above-described components. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming the above-mentioned respective layers thereon, and then depositing a material that can be used as the cathode thereon. In addition to such a method, the organic light emitting device can be manufactured by sequentially depositing the above-described components from a cathode material to an anode material in the reverse order on a substrate (WO 2003/012890). Further, the light emitting layer can be formed using the host and the dopant by a solution coating method as well as a vacuum deposition method. Herein, the solution coating method means a spin coating, a dip coating, a doctor blading, an inkjet printing, a screen printing, a spray method, a roll coating, or the like, but is not limited thereto.

The organic light emitting device according to the present disclosure can be a bottom emission device, a top emission device, or a double-sided light emitting device, and in particular, can be a bottom emission device requiring relatively high luminous efficiency.

Hereinafter, preferred examples of a compound of Chemical Formula 1, a compound of Chemical Formula 2, and an organic light emitting device including the same, and a preparation of them according to the present disclosure are presented to aid in the understanding of the invention. However, these examples are presented for illustrative purposes only, and the scope of the present disclosure is not limited thereto.

### EXAMPLES

### (Preparation of First Compound)

### Synthesis Example 1-1

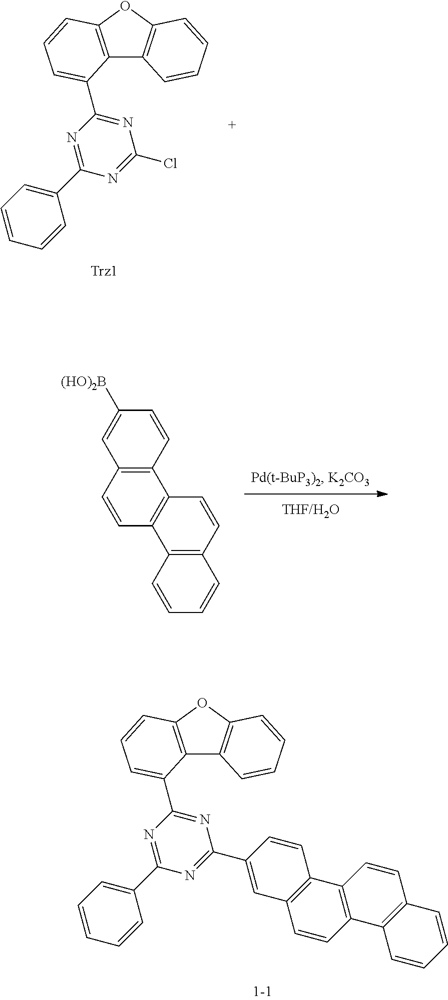 [SMILES: Clc1nc(-c2ccccc2)nc(-c2cccc3oc4ccccc4c23)n1 ; OB(O)c1ccc2c(ccc3c4ccccc4ccc23)c1 ; c1ccc(-c2nc(-c3ccc4c(ccc5c6ccccc6ccc45)c3)nc(-c3cccc4oc5ccccc5c34)n2)cc1]

Compound Trz1 (15 g, 41.9 mmol) and chrysen-2-yl boronic acid (12 g, 44 mmol) were added to 300 mL of tetrahydrofuran (THF), and the mixture was stirred and refluxed. Afterwards, potassium carbonate (K₂CO₃, 17.4 g, 125.8 mmol) was dissolved in 52 mL of water, and then added thereto. Thereafter, it was stirred sufficiently, followed by adding bis(tri-tert-butylphosphine) palladium (0) (Pd(t-BuP₃)₂, 0.2 g, 0.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-1 of 16.3 g. (Yield 71%, MS: [M+H]⁺=550).

### Synthesis Example 1-2

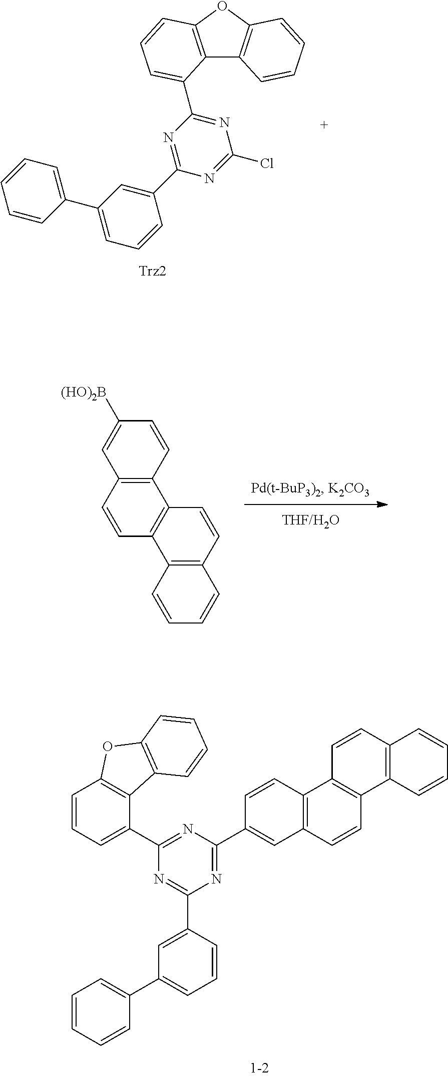 [SMILES: Clc1nc(-c2cccc(-c3ccccc3)c2)nc(-c2cccc3oc4ccccc4c23)n1 ; OB(O)c1ccc2c(ccc3c4ccccc4ccc23)c1 ; c1ccc(-c2cccc(-c3nc(-c4ccc5c(ccc6c7ccccc7ccc56)c4)nc(-c4cccc5oc6ccccc6c45)n3)c2)cc1]

Compound Trz2 (15 g, 34.6 mmol) and chrysen-2-yl boronic acid (9.9 g, 36.3 mmol) were added to 300 mL of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-2 of 15.6 g. (Yield 72%, MS: [M+H]⁺=626).

### Synthesis Example 1-3

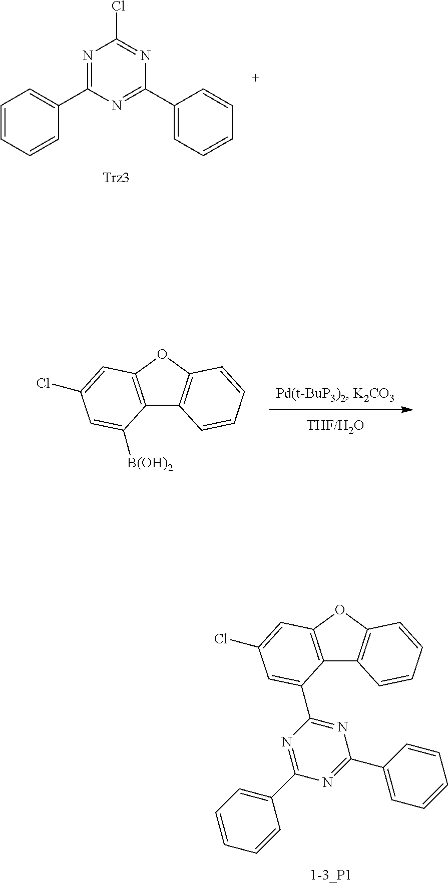 [SMILES: Clc1nc(-c2ccccc2)nc(-c2ccccc2)n1 ; OB(O)c1cc(Cl)cc2oc3ccccc3c12 ; Clc1cc(-c2nc(-c3ccccc3)nc(-c3ccccc3)n2)c2c(c1)oc1ccccc12]

### Step 1) Synthesis of Compound 1-3_P1

Compound Trz3 (15 g, 56 mmol) and (3-chlorodibenzo[b,d]furan-1-yl) boronic acid (14.5 g, 58.8 mmol) were added to 300 mL of tetrahydrofuran (THF), and the mixture was stirred and refluxed. Afterwards, potassium carbonate (23.2 g, 168.1 mmol) was dissolved in 70 ml of water, and then added thereto. Thereafter, it was stirred sufficiently, followed by adding bis(tri-tert-butylphosphine) palladium (0) (0.3 g, 0.6 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-3_P1 of 17.5 g. (Yield 72%, MS: [M+H]⁺=434).

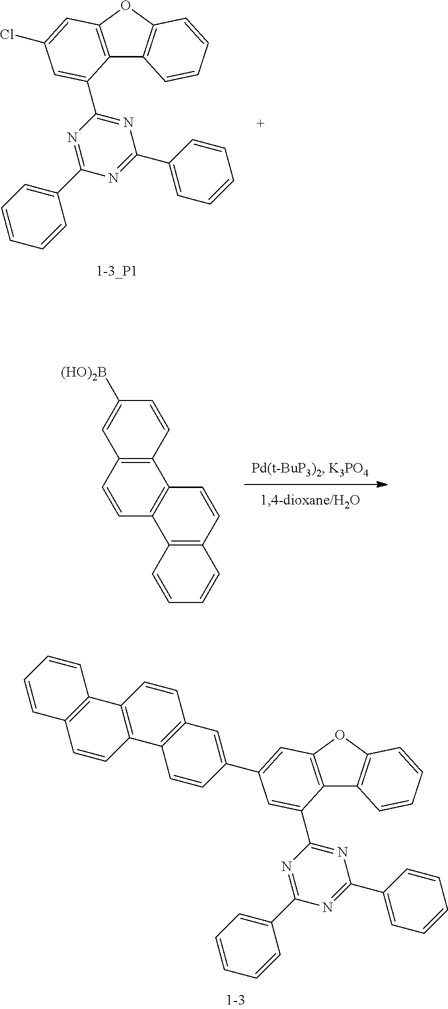 [SMILES: Clc1cc(-c2nc(-c3ccccc3)nc(-c3ccccc3)n2)c2c(c1)oc1ccccc12 ; OB(O)c1ccc2c(ccc3c4ccccc4ccc23)c1 ; c1ccc(-c2nc(-c3ccccc3)nc(-c3cc(-c4ccc5c(ccc6c7ccccc7ccc56)c4)cc4oc5ccccc5c34)n2)cc1]

### Step 2) Synthesis of Compound 1-3

Compound 1-3_P1 (15 g, 34.6 mmol) prepared in the above Step 1) and chrysen-2-yl boronic acid (9.9 g, 36.3 mmol) were added to 300 mL of tetrahydrofuran (THF), and the mixture was stirred and refluxed. Afterwards, potassium phosphate (K₃PO₄, 22 g, 103.7 mmol) was dissolved in 66 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-3 of 15.8 g. (Yield 73%, MS: [M+H]⁺=626).

### Synthesis Example 1-4

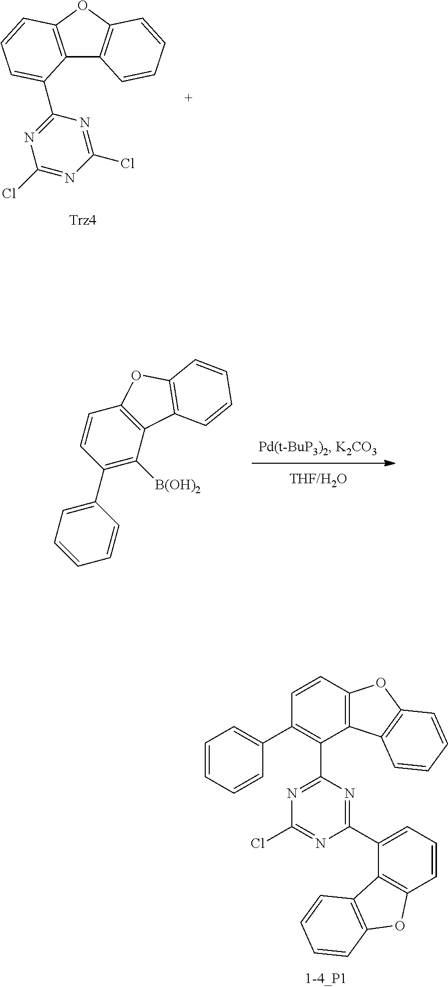 [SMILES: Clc1nc(Cl)nc(-c2cccc3oc4ccccc4c23)n1 ; OB(O)c1c(-c2ccccc2)ccc2oc3ccccc3c12 ; Clc1nc(-c2cccc3oc4ccccc4c23)nc(-c2c(-c3ccccc3)ccc3oc4ccccc4c23)n1]

### Step 1) Synthesis of Compound 1-4_P1

Compound Trz4 (15 g, 47.4 mmol) and (2-phenyldibenzo[b,d]furan-1-yl) boronic acid (14.4 g, 49.8 mmol) were added to 300 mL of tetrahydrofuran (THF), and the mixture was stirred and refluxed. Afterwards, potassium carbonate (19.7 g, 142.3 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-4_P1 of 16.1 g. (Yield 65%, MS: [M+H]⁺=524).

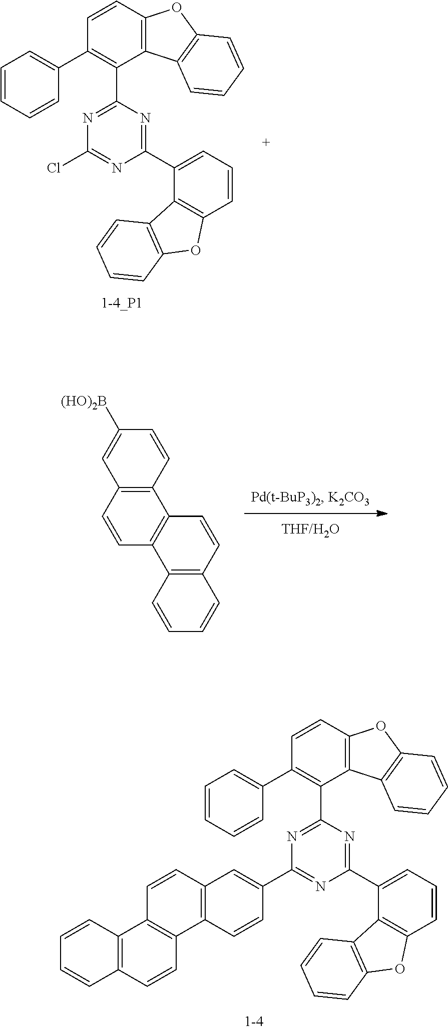 [SMILES: Clc1nc(-c2cccc3oc4ccccc4c23)nc(-c2c(-c3ccccc3)ccc3oc4ccccc4c23)n1 ; OB(O)c1ccc2c(ccc3c4ccccc4ccc23)c1 ; c1ccc(-c2ccc3oc4ccccc4c3c2-c2nc(-c3ccc4c(ccc5c6ccccc6ccc45)c3)nc(-c3cccc4oc5ccccc5c34)n2)cc1]

### Step 2) Synthesis of Compound 1-4

Compound 1-4_P1 (15 g, 28.6 mmol) prepared in the above Step 1) and chrysen-2-yl boronic acid (8.2 g, 30.1 mmol) were added to 300 mL of tetrahydrofuran (THF), and the mixture was stirred and refluxed. Afterwards, potassium carbonate (11.9 g, 85.9 mmol) was dissolved in 36 mL of water, and then added thereto. Thereafter, it was stirred sufficiently, followed by adding bis(tri-tert-butylphosphine) palladium (0) (0.1 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-4 of 13.1 g. (Yield 64%, MS: [M+H]⁺=716).

### Synthesis Example 1-5

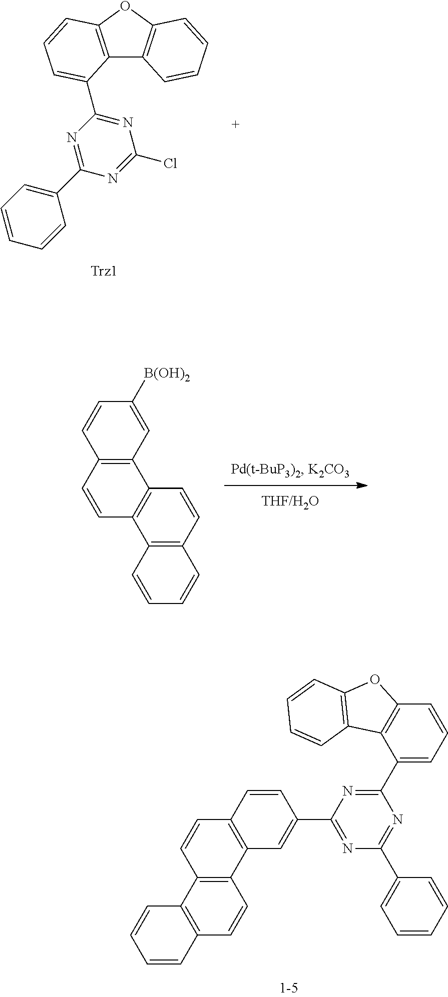 [SMILES: Clc1nc(-c2ccccc2)nc(-c2cccc3oc4ccccc4c23)n1 ; OB(O)c1ccc2ccc3c4ccccc4ccc3c2c1 ; c1ccc(-c2nc(-c3ccc4ccc5c6ccccc6ccc5c4c3)nc(-c3cccc4oc5ccccc5c34)n2)cc1]

Compound Trz1 (15 g, 41.9 mmol) and chrysen-3-yl boronic acid (12 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-5 of 15.9 g. (Yield 69%, MS: [M+H]⁺=550).

### Synthesis Example 1-6

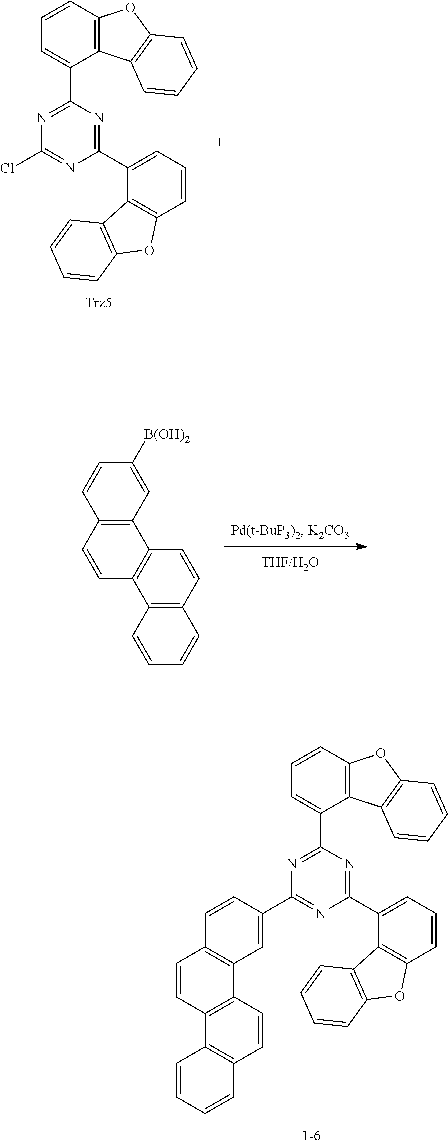 [SMILES: Clc1nc(-c2cccc3oc4ccccc4c23)nc(-c2cccc3oc4ccccc4c23)n1 ; OB(O)c1ccc2ccc3c4ccccc4ccc3c2c1 ; c1ccc2c(c1)ccc1c3cc(-c4nc(-c5cccc6oc7ccccc7c56)nc(-c5cccc6oc7ccccc7c56)n4)ccc3ccc21]

Compound Trz5 (15 g, 33.5 mmol) and chrysen-3-yl boronic acid (9.6 g, 35.2 mmol) were added to 300 mL of tetrahydrofuran (THF), and the mixture was stirred and refluxed. Afterwards, potassium carbonate (13.9 g, 100.5 mmol) was dissolved in 42 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-6 of 15 g. (Yield 70%, MS: [M+H]⁺=640).

### Synthesis Example 1-7

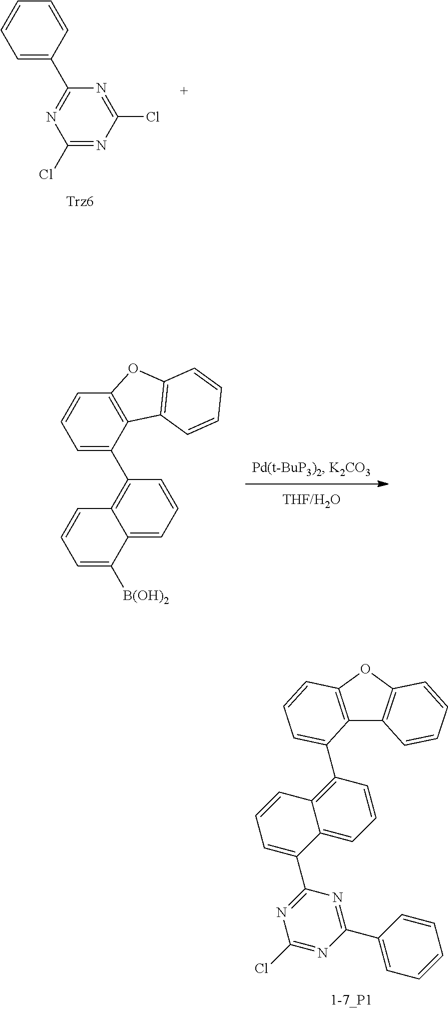 [SMILES: Clc1nc(Cl)nc(-c2ccccc2)n1 ; OB(O)c1cccc2c(-c3cccc4oc5ccccc5c34)cccc12 ; Clc1nc(-c2ccccc2)nc(-c2cccc3c(-c4cccc5oc6ccccc6c45)cccc23)n1]

### Step 1) Synthesis of Compound 1-7_P1

Compound Trz6 (15 g, 66.4 mmol) and (5-(dibenzo[b,d]furan-1-yl)naphthalen-1-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-7_P1 of 20.8 g. (Yield 65%, MS: [M+H]⁺=484).

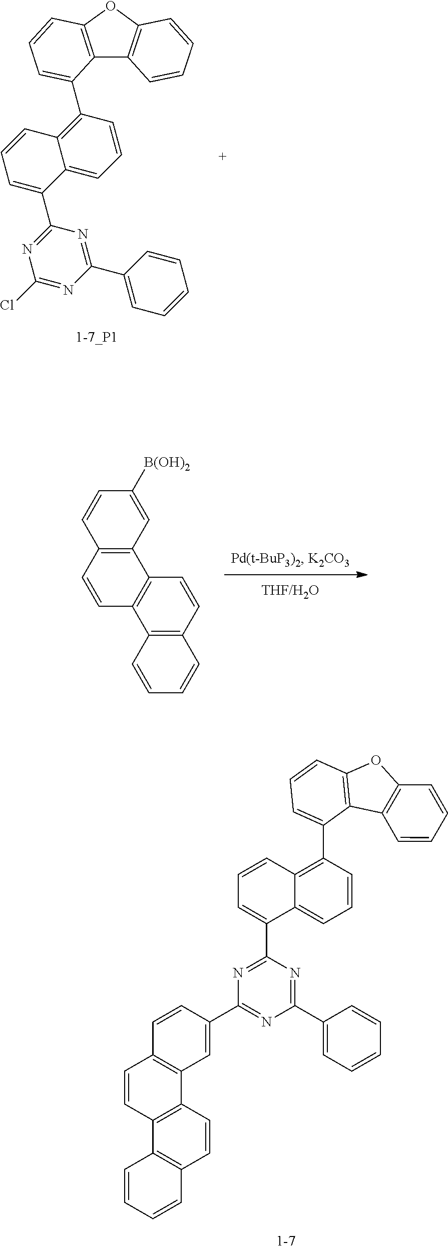 [SMILES: Clc1nc(-c2ccccc2)nc(-c2cccc3c(-c4cccc5oc6ccccc6c45)cccc23)n1 ; OB(O)c1ccc2ccc3c4ccccc4ccc3c2c1 ; c1ccc(-c2nc(-c3ccc4ccc5c6ccccc6ccc5c4c3)nc(-c3cccc4c(-c5cccc6oc7ccccc7c56)cccc34)n2)cc1]

### Step 2) Synthesis of Compound 1-7

Compound 1-7_P1 (15 g, 31 mmol) prepared in the above Step 1) and chrysen-3-yl boronic acid (11 g, 32.5 mmol) were added to 300 mL of tetrahydrofuran (THF), and the mixture was stirred and refluxed. Afterwards, potassium carbonate (12.9 g, 93 mmol) was dissolved in 36 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-7 of 38.2 g. (Yield 66%, MS: [M+H]⁺=675).

### Synthesis Example 1-8

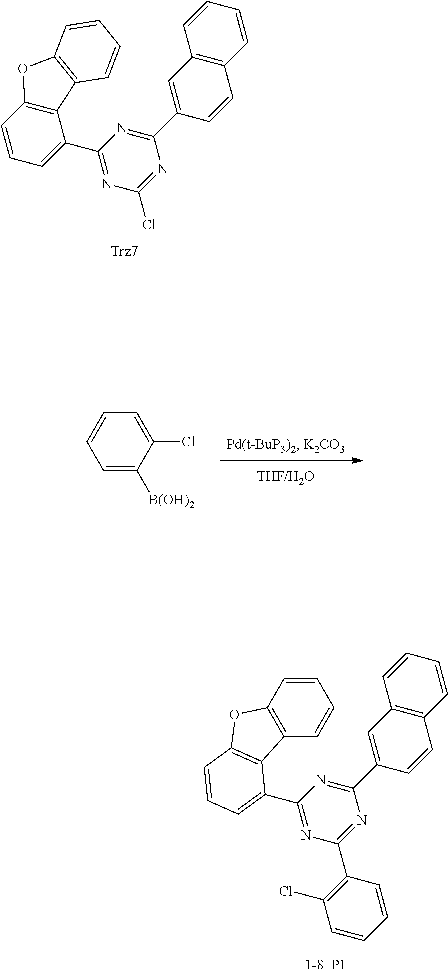 [SMILES: Clc1nc(-c2ccc3ccccc3c2)nc(-c2cccc3oc4ccccc4c23)n1 ; OB(O)c1ccccc1Cl ; Clc1ccccc1-c1nc(-c2ccc3ccccc3c2)nc(-c2cccc3oc4ccccc4c23)n1]

### Step 1) Synthesis of Compound 1-8_P1

Compound Trz7 (15 g, 36.8 mmol) and (2-chlorophenyl) boronic acid (6 g, 38.6 mmol) were added to 300 mL of tetrahydrofuran (THF), and the mixture was stirred and refluxed. Afterwards, potassium carbonate (15.2 g, 110.3 mmol) was dissolved in 46 ml of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-8_P1 of 12.8 g. (Yield 72%, MS: [M+H]⁺=484).

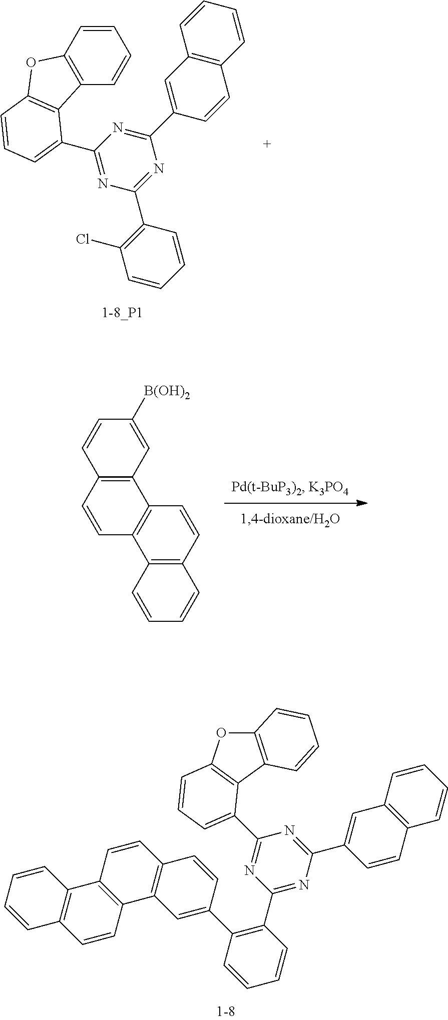 [SMILES: Clc1ccccc1-c1nc(-c2ccc3ccccc3c2)nc(-c2cccc3oc4ccccc4c23)n1 ; OB(O)c1ccc2ccc3c4ccccc4ccc3c2c1 ; c1ccc(-c2nc(-c3ccc4ccccc4c3)nc(-c3cccc4oc5ccccc5c34)n2)c(-c2ccc3ccc4c5ccccc5ccc4c3c2)c1]

### Step 2) Synthesis of Compound 1-8

Compound 1-8_P1 (15 g, 31 mmol) prepared in the above Step 1) and chrysen-3-yl boronic acid (8.9 g, 32.5 mmol) were added to 300 ml of 1,4-dioxane, and the mixture was stirred and refluxed. Afterwards, potassium phosphate (19.7 g, 93 mmol) was dissolved in 59 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-8 of 15.1 g. (Yield 72%, MS: [M+H]⁺=676).

### Synthesis Example 1-9

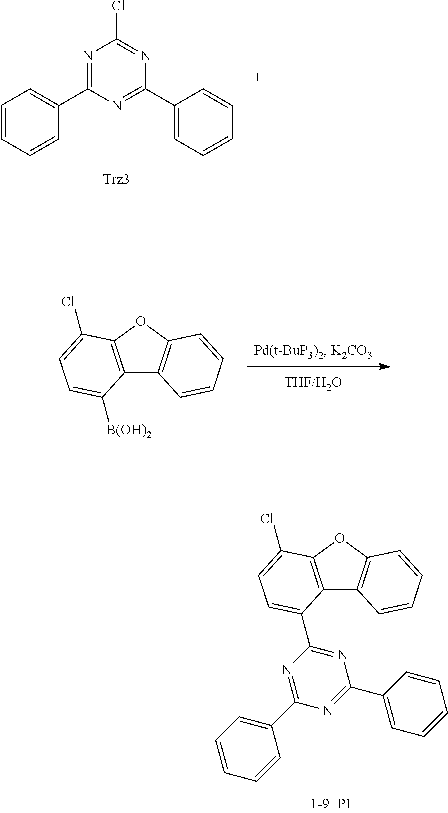 [SMILES: Clc1nc(-c2ccccc2)nc(-c2ccccc2)n1 ; OB(O)c1ccc(Cl)c2oc3ccccc3c12 ; Clc1ccc(-c2nc(-c3ccccc3)nc(-c3ccccc3)n2)c2c1oc1ccccc12]

### Step 1) Synthesis of Compound 1-9_P1

Compound Trz3 (15 g, 56 mmol) and (4-chlorodibenzo[b,d]furan-1-yl) boronic acid (14.5 g, 58.8 mmol) were added to 300 mL of tetrahydrofuran (THF), and the mixture was stirred and refluxed. Afterwards, potassium carbonate (23.2 g, 168.1 mmol) was dissolved in 70 ml of water, and then added thereto. Thereafter, it was stirred sufficiently, followed by adding bis(tri-tert-butylphosphine) palladium (0) (0.3 g, 0.6 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-9_P1 of 14.8 g. (Yield 61%, MS: [M+H]⁺=434).

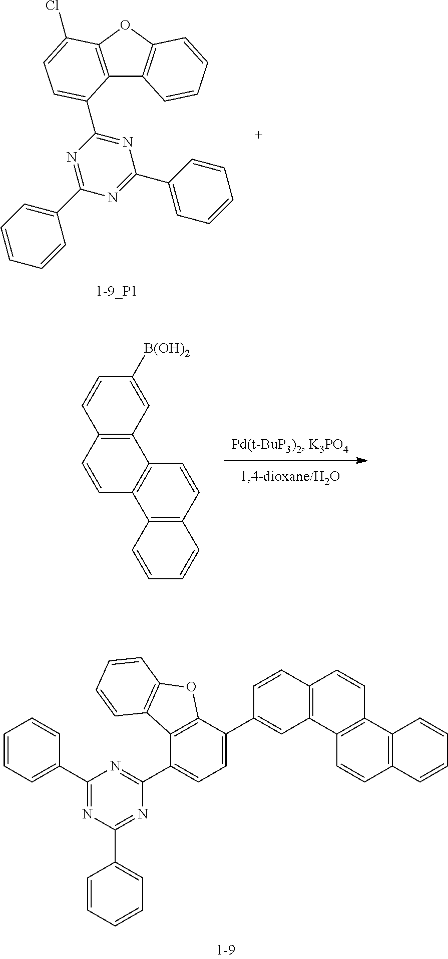 [SMILES: Clc1ccc(-c2nc(-c3ccccc3)nc(-c3ccccc3)n2)c2c1oc1ccccc12 ; OB(O)c1ccc2ccc3c4ccccc4ccc3c2c1 ; c1ccc(-c2nc(-c3ccccc3)nc(-c3ccc(-c4ccc5ccc6c7ccccc7ccc6c5c4)c4oc5ccccc5c34)n2)cc1]

### Step 2) Synthesis of Compound 1-9

Compound 1-9_P1 (15 g, 31 mmol) prepared in the above Step 1) and chrysen-3-yl boronic acid (8.9 g, 32.5 mmol) were added to 300 ml of 1,4-dioxane, and the mixture was stirred and refluxed. Afterwards, potassium phosphate (19.7 g, 93 mmol) was dissolved in 59 ml of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-9 of 12.8 g. (Yield 66%, MS: [M+H]⁺=626).

### Synthesis Example 1-10

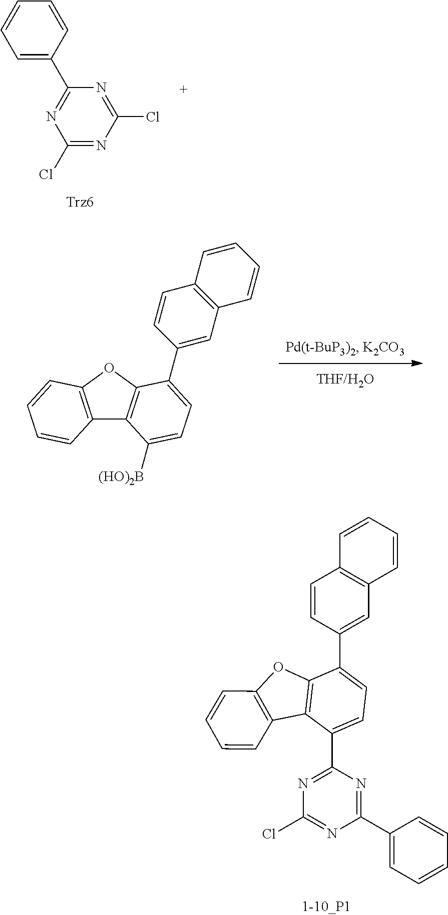 [SMILES: Clc1nc(Cl)nc(-c2ccccc2)n1 ; OB(O)c1ccc(-c2ccc3ccccc3c2)c2oc3ccccc3c12 ; Clc1nc(-c2ccccc2)nc(-c2ccc(-c3ccc4ccccc4c3)c3oc4ccccc4c23)n1]

### Step 1) Synthesis of Compound 1-10_P1

Compound Trz6 (15 g, 66.4 mmol) and (4-(naphthalen-2-yl)dibenzo[b,d]furan-1-yl) boronic acid (23.6 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-10_P1 of 21.5 g. (Yield 67%, MS: [M+H]⁺=484).

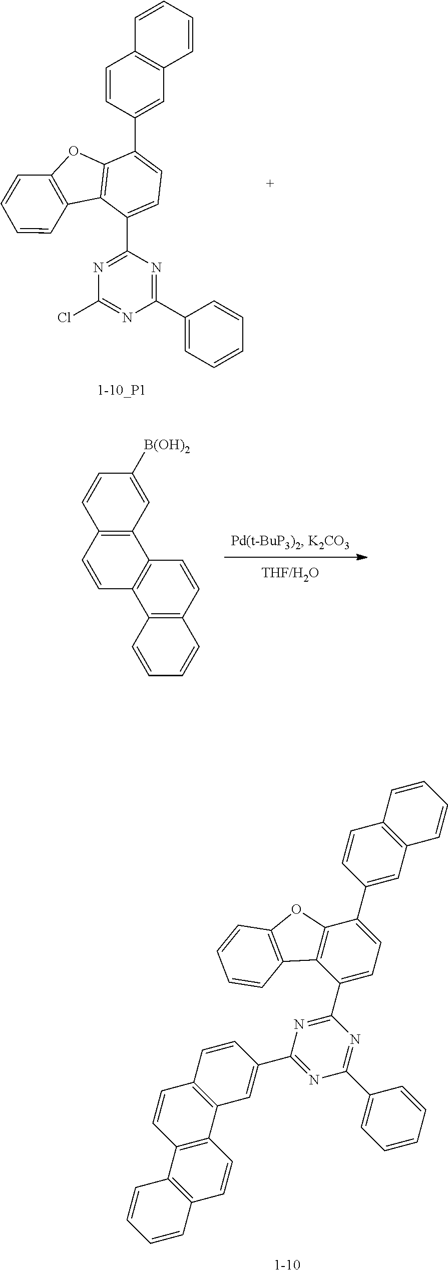 [SMILES: Clc1nc(-c2ccccc2)nc(-c2ccc(-c3ccc4ccccc4c3)c3oc4ccccc4c23)n1 ; OB(O)c1ccc2ccc3c4ccccc4ccc3c2c1 ; c1ccc(-c2nc(-c3ccc4ccc5c6ccccc6ccc5c4c3)nc(-c3ccc(-c4ccc5ccccc5c4)c4oc5ccccc5c34)n2)cc1]

### Step 2) Synthesis of Compound 1-10

Compound 1-10_P1 (15 g, 31 mmol) prepared in the above Step 1) and chrysen-3-yl boronic acid (8.9 g, 32.5 mmol) were added to 300 mL of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-10 of 15.1 g. (Yield 72%, MS: [M+H]⁺=676).

### Synthesis Example 1-11

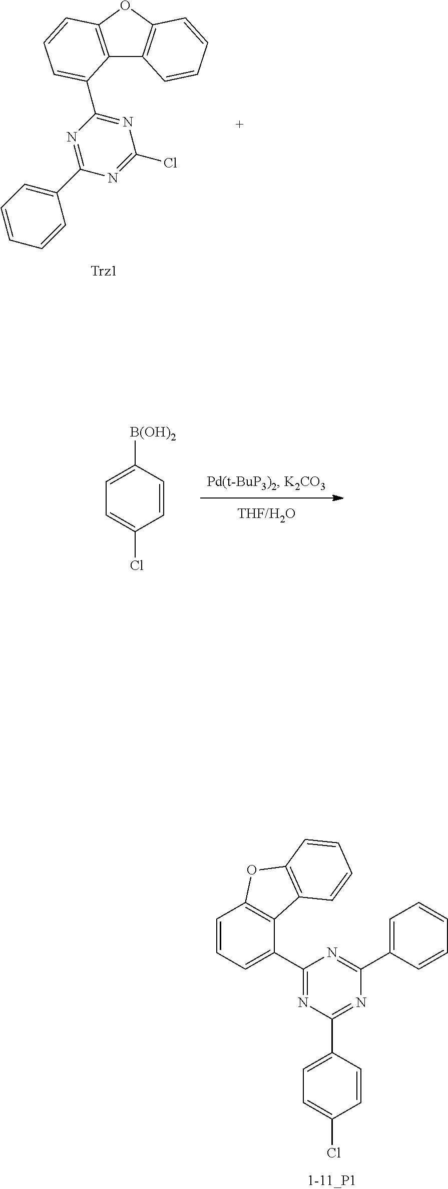 [SMILES: Clc1nc(-c2ccccc2)nc(-c2cccc3oc4ccccc4c23)n1 ; OB(O)c1ccc(Cl)cc1 ; Clc1ccc(-c2nc(-c3ccccc3)nc(-c3cccc4oc5ccccc5c34)n2)cc1]

### Step 1) Synthesis of Compound 1-11_P1

Compound Trz1 (15 g, 41.9 mmol) and (4-chlorophenyl) boronic acid (6.9 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-11_P1 of 12.3 g. (Yield 68%, MS: [M+H]⁺=434).

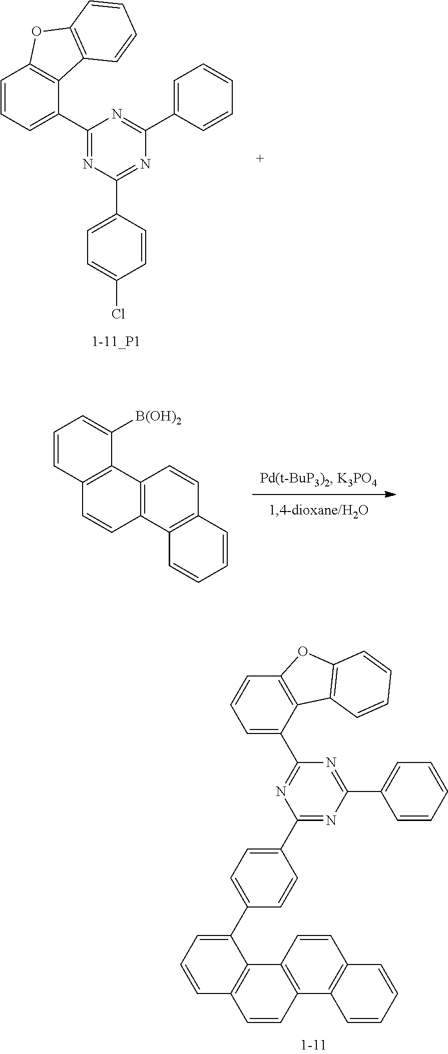 [SMILES: Clc1ccc(-c2nc(-c3ccccc3)nc(-c3cccc4oc5ccccc5c34)n2)cc1 ; OB(O)c1cccc2ccc3c4ccccc4ccc3c12 ; c1ccc(-c2nc(-c3ccc(-c4cccc5ccc6c7ccccc7ccc6c45)cc3)nc(-c3cccc4oc5ccccc5c34)n2)cc1]

### Step 2) Synthesis of Compound 1-11

Compound 1-11_P1 (165 g, 341.7 mmol) prepared in the above Step 1) and chrysen-4-yl boronic acid (97.6 g, 358.8 mmol) were added to 300 mL of 1,4-dioxane, and the mixture was stirred and refluxed. Afterwards, potassium phosphate (217.6 g, 1025.1 mmol) was dissolved in 653 ml of water, and then added thereto. Thereafter, it was stirred sufficiently, followed by adding bis(tri-tert-butylphosphine) palladium (0) (1.7 g, 3.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-11 of 160.2 g. (Yield 75%, MS: [M+H]⁺=626).

### Synthesis Example 1-12

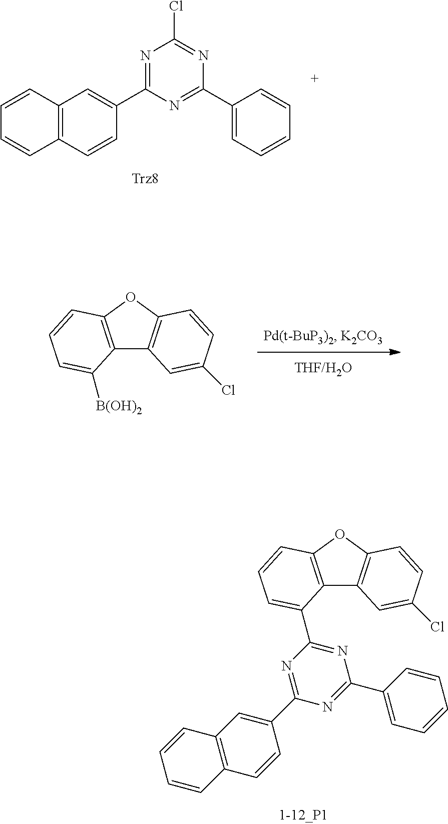 [SMILES: Clc1nc(-c2ccccc2)nc(-c2ccc3ccccc3c2)n1 ; OB(O)c1cccc2oc3ccc(Cl)cc3c12 ; Clc1ccc2oc3cccc(-c4nc(-c5ccccc5)nc(-c5ccc6ccccc6c5)n4)c3c2c1]

### Step 1) Synthesis of Compound 1-12_P1

Compound Trz8 (15 g, 47.2 mmol) and (8-chlorodibenzo[b,d]furan-1-yl) boronic acid (12.2 g, 49.6 mmol) were added to 300 mL of tetrahydrofuran (THF), and the mixture was stirred and refluxed. Afterwards, potassium carbonate (19.6 g, 141.6 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-12_P1 of 15.8 g. (Yield 69%, MS: [M+H]⁺=485).

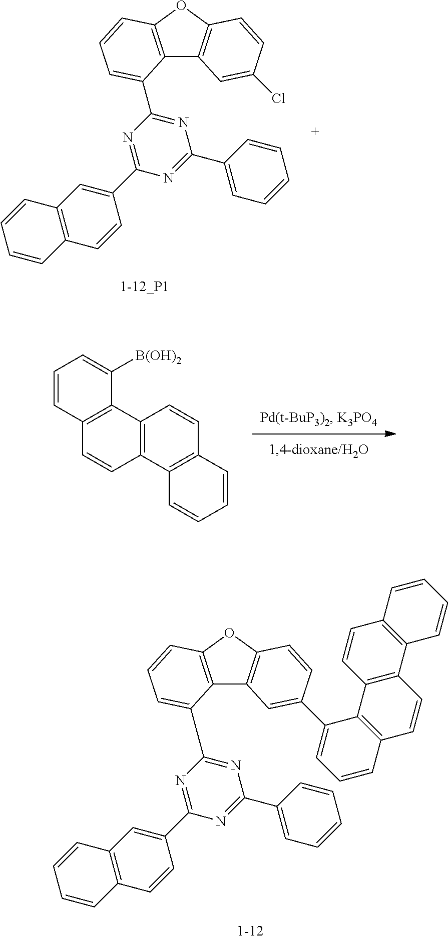 [SMILES: Clc1ccc2oc3cccc(-c4nc(-c5ccccc5)nc(-c5ccc6ccccc6c5)n4)c3c2c1 ; OB(O)c1cccc2ccc3c4ccccc4ccc3c12 ; c1ccc(-c2nc(-c3ccc4ccccc4c3)nc(-c3cccc4oc5ccc(-c6cccc7ccc8c9ccccc9ccc8c67)cc5c34)n2)cc1]

### Step 2) Synthesis of Compound 1-12

Compound 1-12_P1 (15 g, 31 mmol) prepared in the above Step 1) and chrysen-4-yl boronic acid (8.9 g, 32.5 mmol) were added to 300 ml of 1,4-dioxane, and the mixture was stirred and refluxed. Afterwards, potassium phosphate (19.7 g, 93 mmol) was dissolved in 59 ml of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-12 of 13.2 g. (Yield 63%, MS: [M+H]⁺=676).

### Synthesis Example 1-13

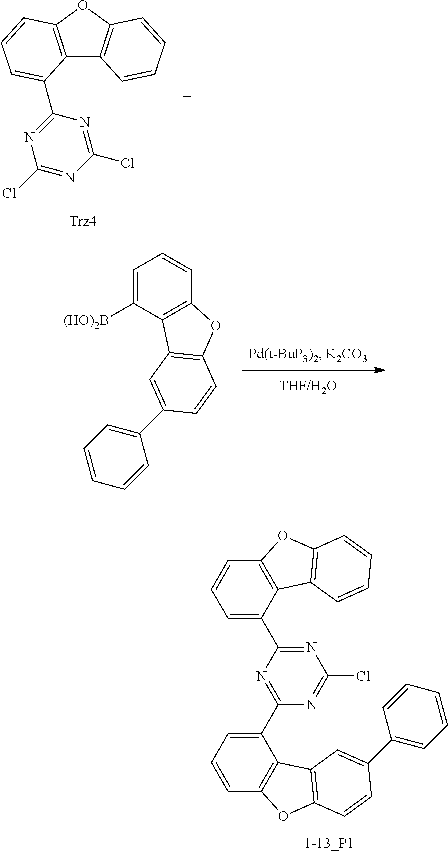 [SMILES: Clc1nc(Cl)nc(-c2cccc3oc4ccccc4c23)n1 ; OB(O)c1cccc2oc3ccc(-c4ccccc4)cc3c12 ; Clc1nc(-c2cccc3oc4ccccc4c23)nc(-c2cccc3oc4ccc(-c5ccccc5)cc4c23)n1]

### Step 1) Synthesis of Compound 1-13_P1

Compound Trz4 (15 g, 47.4 mmol) and (8-phenyldibenzo[b,d]furan-1-yl) boronic acid (14.4 g, 49.8 mmol) were added to 300 mL of tetrahydrofuran (THF), and the mixture was stirred and refluxed. Afterwards, potassium carbonate (19.7 g, 142.3 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-13_P1 of 18.6 g. (Yield 75%, MS: [M+H]⁺=524).

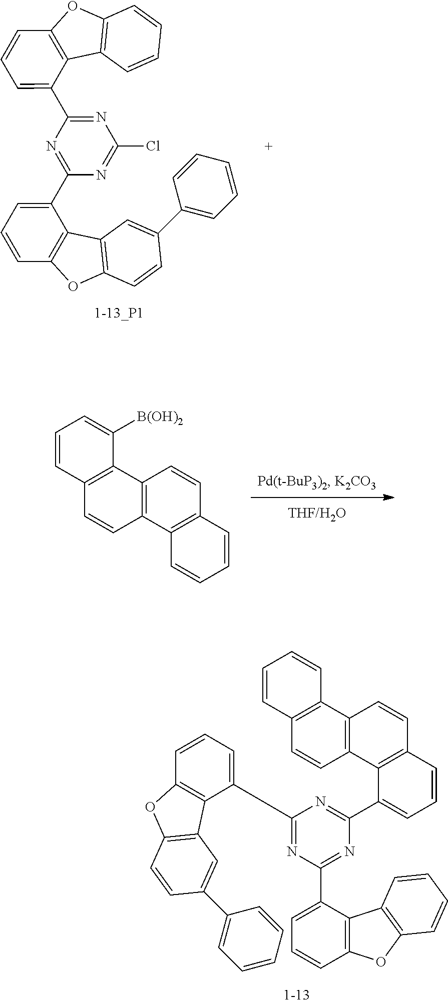 [SMILES: Clc1nc(-c2cccc3oc4ccccc4c23)nc(-c2cccc3oc4ccc(-c5ccccc5)cc4c23)n1 ; OB(O)c1cccc2ccc3c4ccccc4ccc3c12 ; c1ccc(-c2ccc3oc4cccc(-c5nc(-c6cccc7ccc8c9ccccc9ccc8c67)nc(-c6cccc7oc8ccccc8c67)n5)c4c3c2)cc1]

### Step 2) Synthesis of Compound 1-13

Compound 1-13_P1 (15 g, 28.6 mmol) prepared in the above Step 1) and chrysen-4-yl boronic acid (8.9 g, 32.5 mmol) were added to 300 mL of tetrahydrofuran (THF), and the mixture was stirred and refluxed. Afterwards, potassium carbonate (11.9 g, 85.9 mmol) was dissolved in 36 ml of water, and then added thereto. Thereafter, it was stirred sufficiently, followed by adding bis(tri-tert-butylphosphine) palladium (0) (0.1 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-13 of 13.1 g. (Yield 64%, MS: [M+H]⁺=716).

### Synthesis Example 1-14

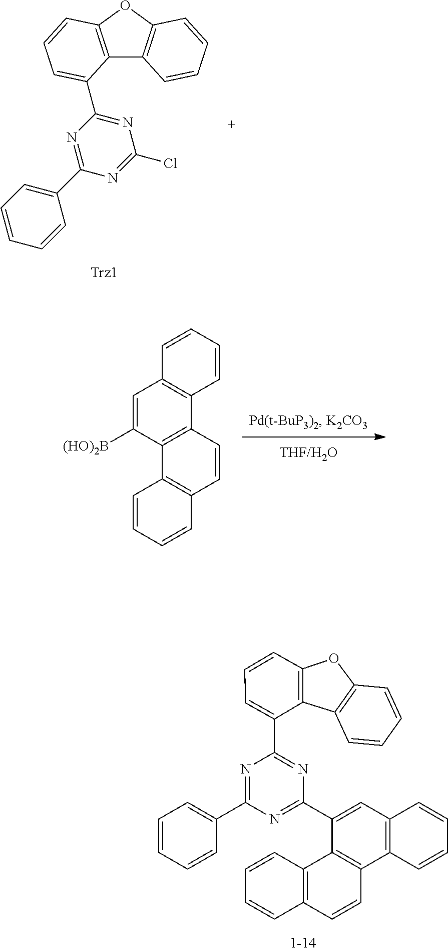 [SMILES: Clc1nc(-c2ccccc2)nc(-c2cccc3oc4ccccc4c23)n1 ; OB(O)c1cc2ccccc2c2ccc3ccccc3c12 ; c1ccc(-c2nc(-c3cccc4oc5ccccc5c34)nc(-c3cc4ccccc4c4ccc5ccccc5c34)n2)cc1]

Compound Trz1 (15 g, 41.9 mmol) and chrysen-5-yl boronic acid (12 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-14 of 15.2 g. (Yield 66%, MS: [M+H]⁺=550).

### Synthesis Example 1-15

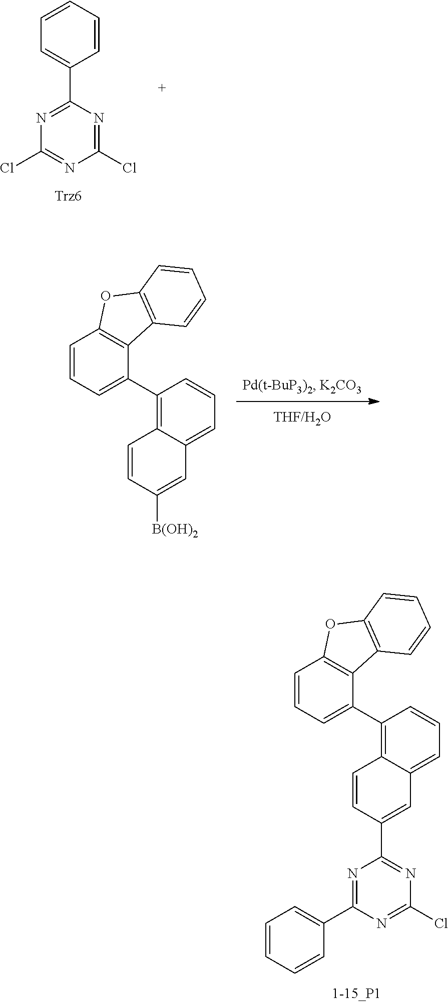 [SMILES: Clc1nc(Cl)nc(-c2ccccc2)n1 ; OB(O)c1ccc2c(-c3cccc4oc5ccccc5c34)cccc2c1 ; Clc1nc(-c2ccccc2)nc(-c2ccc3c(-c4cccc5oc6ccccc6c45)cccc3c2)n1]

### Step 1) Synthesis of Compound 1-15_P1

Compound Trz6 (15 g, 66.4 mmol) and (5-(dibenzo[b,d]furan-1-yl)naphthalen-2-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-15_P1 of 19.6 g. (Yield 61%, MS: [M+H]⁺=484).

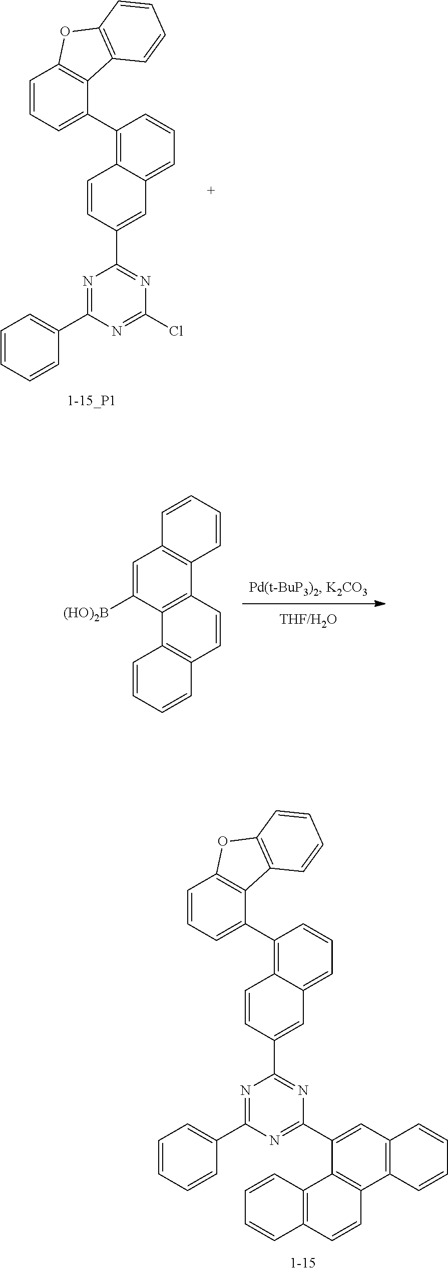 [SMILES: Clc1nc(-c2ccccc2)nc(-c2ccc3c(-c4cccc5oc6ccccc6c45)cccc3c2)n1 ; OB(O)c1cc2ccccc2c2ccc3ccccc3c12 ; c1ccc(-c2nc(-c3ccc4c(-c5cccc6oc7ccccc7c56)cccc4c3)nc(-c3cc4ccccc4c4ccc5ccccc5c34)n2)cc1]

### Step 2) Synthesis of Compound 1-15

Compound 1-15_P1 (15 g, 31 mmol) prepared in the above Step 1) and chrysen-5-yl boronic acid (8.9 g, 32.5 mmol) were added to 300 mL of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-15 of 13.4 g. (Yield 64%, MS: [M+H]⁺=676).

### Synthesis Example 1-16

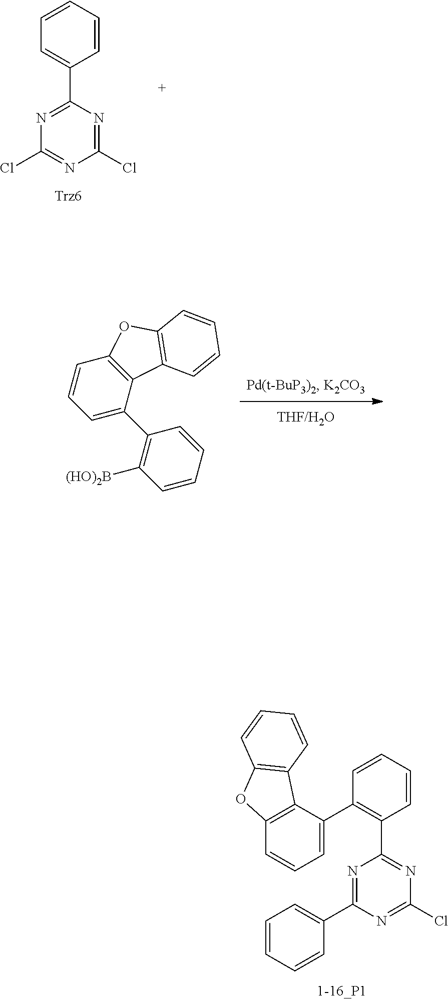 [SMILES: Clc1nc(Cl)nc(-c2ccccc2)n1 ; OB(O)c1ccccc1-c1cccc2oc3ccccc3c12 ; Clc1nc(-c2ccccc2)nc(-c2ccccc2-c2cccc3oc4ccccc4c23)n1]

### Step 1) Synthesis of Compound 1-16_P1

Compound Trz6 (15 g, 66.4 mmol) and ((2-(dibenzo[b,d]furan-1-yl)phenyl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 ml of water, and then added thereto. Thereafter, it was stirred sufficiently, followed by adding bis(tri-tert-butylphosphine) palladium (0) (0.3 g, 0.7 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-16_P1 of 19.3 g. (Yield 67%, MS: [M+H]⁺=434).

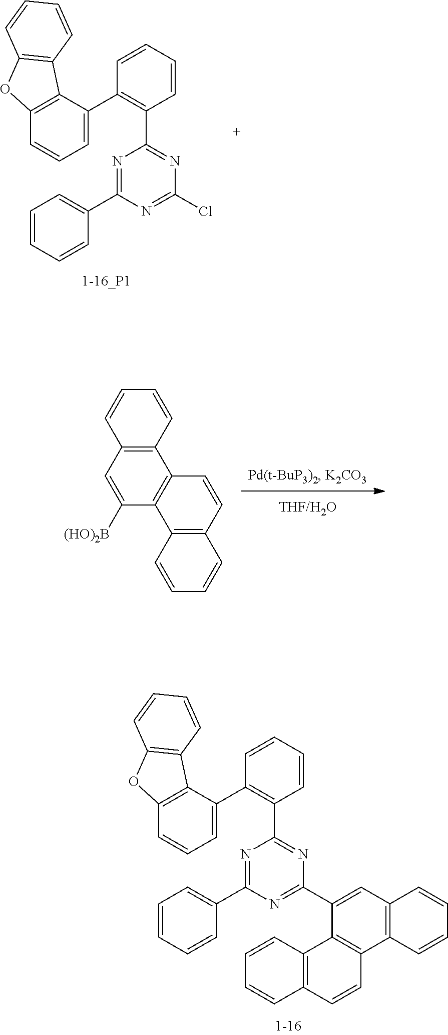 [SMILES: Clc1nc(-c2ccccc2)nc(-c2ccccc2-c2cccc3oc4ccccc4c23)n1 ; OB(O)c1cc2ccccc2c2ccc3ccccc3c12 ; c1ccc(-c2nc(-c3ccccc3-c3cccc4oc5ccccc5c34)nc(-c3cc4ccccc4c4ccc5ccccc5c34)n2)cc1]

### Step 2) Synthesis of Compound 1-16

Compound 1-16_P1 (15 g, 34.6 mmol) prepared in the above Step 1) and chrysen-5-yl boronic acid (9.9 g, 36.3 mmol) were added to 300 mL of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-16 of 16.2 g. (Yield 75%, MS: [M+H]⁺=626).

### Synthesis Example 1-17

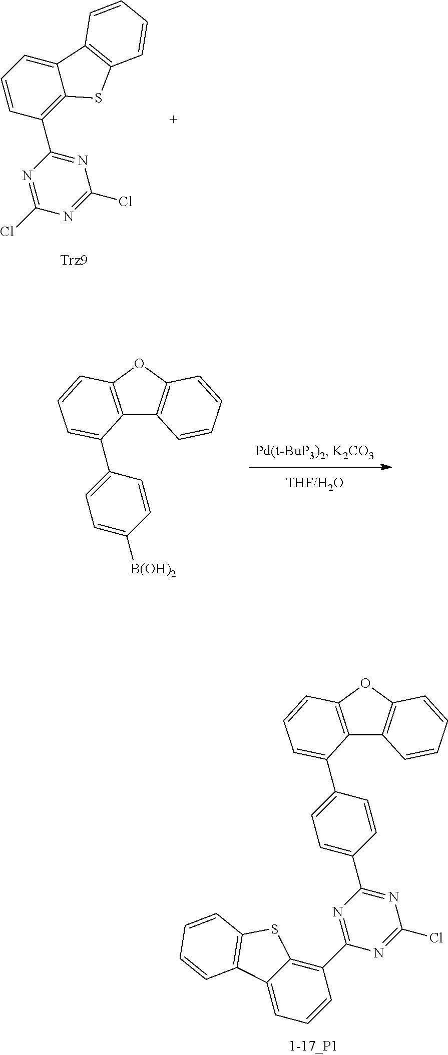 [SMILES: Clc1nc(Cl)nc(-c2cccc3c2sc2ccccc23)n1 ; OB(O)c1ccc(-c2cccc3oc4ccccc4c23)cc1 ; Clc1nc(-c2ccc(-c3cccc4oc5ccccc5c34)cc2)nc(-c2cccc3c2sc2ccccc23)n1]

### Step 1) Synthesis of Compound 1-17_P1

Compound Trz9 (15 g, 45.2 mmol) and (4-(dibenzo[b,d]furan-1-yl)phenyl) boronic acid (13.7 g, 47.4 mmol) were added to 300 mL of tetrahydrofuran (THF), and the mixture was stirred and refluxed. Afterwards, potassium carbonate (18.7 g, 135.5 mmol) was dissolved in 56 mL of water, and then added thereto. Thereafter, it was stirred sufficiently, followed by adding bis(tri-tert-butylphosphine) palladium (0) (0.2 g, 0.5 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-17_P1 of 18.3 g. (Yield 75%, MS: [M+H]⁺=540).

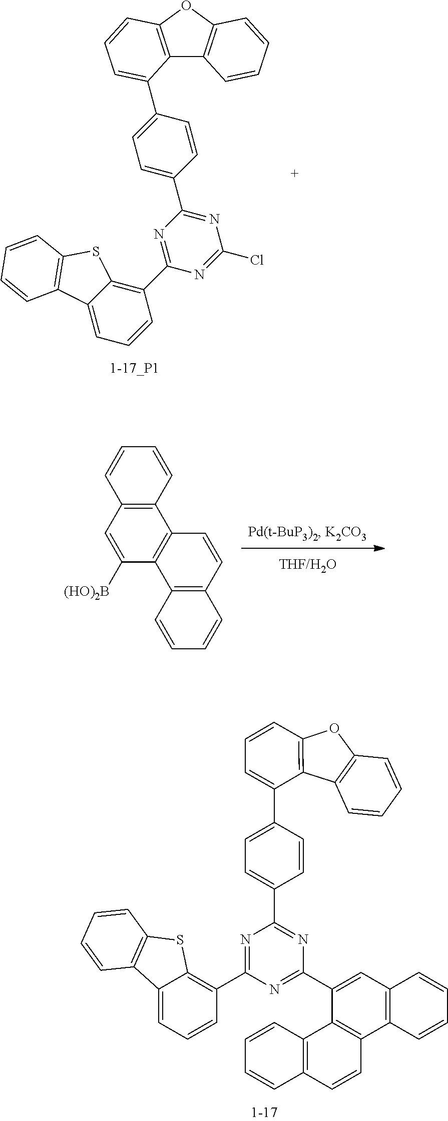 [SMILES: Clc1nc(-c2ccc(-c3cccc4oc5ccccc5c34)cc2)nc(-c2cccc3c2sc2ccccc23)n1 ; OB(O)c1cc2ccccc2c2ccc3ccccc3c12 ; c1ccc2c(c1)cc(-c1nc(-c3ccc(-c4cccc5oc6ccccc6c45)cc3)nc(-c3cccc4c3sc3ccccc34)n1)c1c3ccccc3ccc21]

### Step 2) Synthesis of Compound 1-17

Compound 1-17_P1 (15 g, 27.8 mmol) prepared in the above Step 1) and chrysen-5-yl boronic acid (7.9 g, 29.2 mmol) were added to 300 mL of tetrahydrofuran (THF), and the mixture was stirred and refluxed. Afterwards, potassium carbonate (11.5 g, 83.3 mmol) was dissolved in 35 ml of water, and then added thereto. Thereafter, it was stirred sufficiently, followed by adding bis(tri-tert-butylphosphine) palladium (0) (0.1 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-17 of 13.4 g. (Yield 66%, MS: [M+H]⁺=732).

### Synthesis Example 1-18

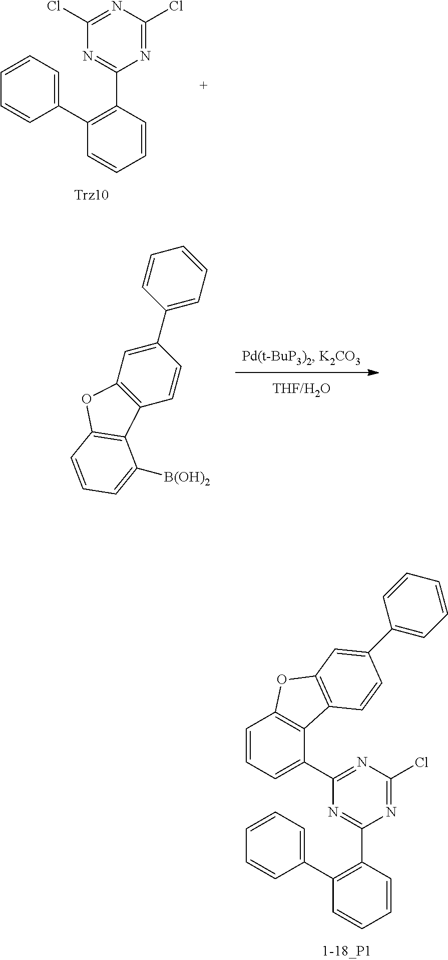 [SMILES: Clc1nc(Cl)nc(-c2ccccc2-c2ccccc2)n1 ; OB(O)c1cccc2oc3cc(-c4ccccc4)ccc3c12 ; Clc1nc(-c2ccccc2-c2ccccc2)nc(-c2cccc3oc4cc(-c5ccccc5)ccc4c23)n1]

### Step 1) Synthesis of Compound 1-18_P1

Compound Trz10 (15 g, 49.6 mmol) and (7-phenyldibenzo[b,d]furan-1-yl) boronic acid (15 g, 52.1 mmol) were added to 300 mL of tetrahydrofuran (THF), and the mixture was stirred and refluxed. Afterwards, potassium carbonate (20.6 g, 148.9 mmol) was dissolved in 62 mL of water, and then added thereto. Thereafter, it was stirred sufficiently, followed by adding bis(tri-tert-butylphosphine) palladium (0) (0.3 g, 0.5 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-18_P1 of 16.4 g. (Yield 65%, MS: [M+H]⁺=510).

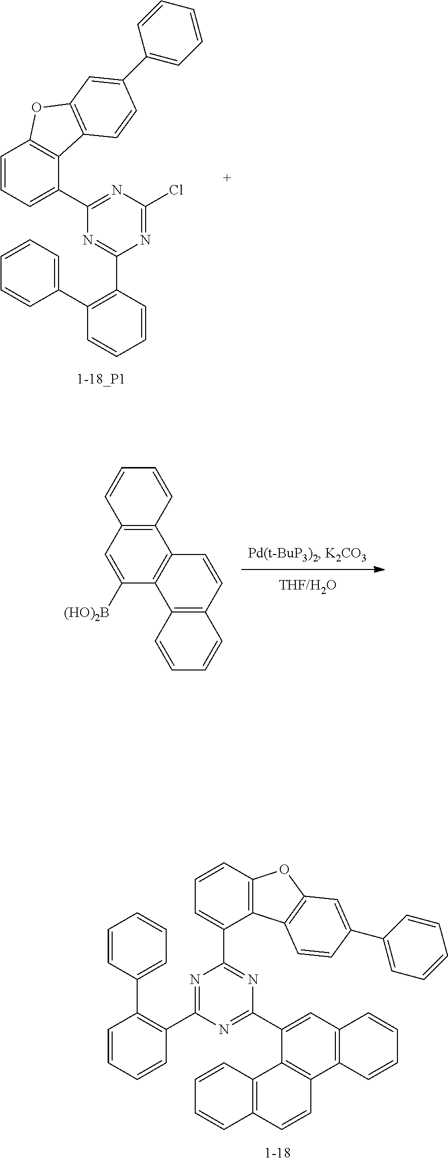 [SMILES: Clc1nc(-c2ccccc2-c2ccccc2)nc(-c2cccc3oc4cc(-c5ccccc5)ccc4c23)n1 ; OB(O)c1cc2ccccc2c2ccc3ccccc3c12 ; c1ccc(-c2ccc3c(c2)oc2cccc(-c4nc(-c5ccccc5-c5ccccc5)nc(-c5cc6ccccc6c6ccc7ccccc7c56)n4)c23)cc1]

### Step 2) Synthesis of Compound 1-18

Compound 1-18_P1 (15 g, 29.4 mmol) prepared in the above Step 1) and chrysen-5-yl boronic acid (8.4 g, 30.9 mmol) were added to 300 mL of tetrahydrofuran (THF), and the mixture was stirred and refluxed. Afterwards, potassium carbonate (12.2 g, 88.2 mmol) was dissolved in 37 ml of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-18 of 13 g. (Yield 63%, MS: [M+H]⁺=702).

### Synthesis Example 1-19

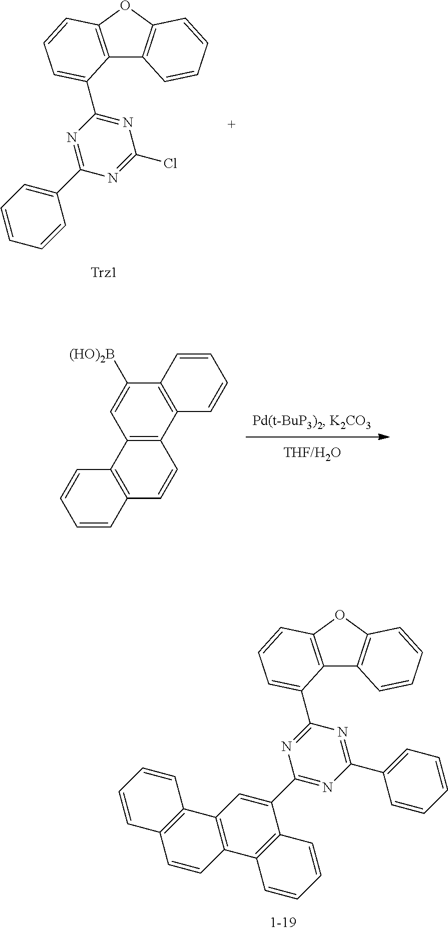 [SMILES: Clc1nc(-c2ccccc2)nc(-c2cccc3oc4ccccc4c23)n1 ; OB(O)c1cc2c3ccccc3ccc2c2ccccc12 ; c1ccc(-c2nc(-c3cc4c5ccccc5ccc4c4ccccc34)nc(-c3cccc4oc5ccccc5c34)n2)cc1]

Compound Trz1 (15 g, 41.9 mmol) and chrysen-6-yl boronic acid (12 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-19 of 15.4 g. (Yield 67%, MS: [M+H]⁺=550).

### Synthesis Example 1-20

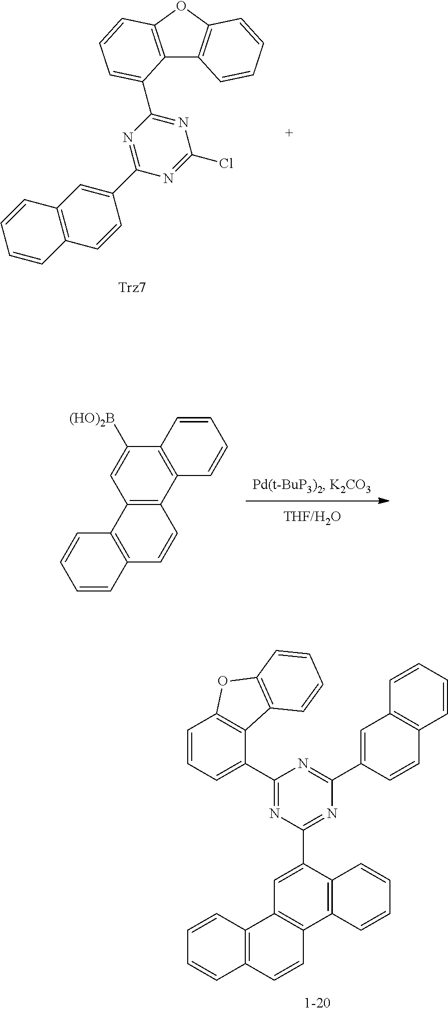 [SMILES: Clc1nc(-c2ccc3ccccc3c2)nc(-c2cccc3oc4ccccc4c23)n1 ; OB(O)c1cc2c3ccccc3ccc2c2ccccc12 ; c1ccc2cc(-c3nc(-c4cc5c6ccccc6ccc5c5ccccc45)nc(-c4cccc5oc6ccccc6c45)n3)ccc2c1]

Compound Trz7 (15 g, 36.8 mmol) and chrysen-6-yl boronic acid (10.5 g, 38.6 mmol) were added to 300 mL of tetrahydrofuran (THF), and the mixture was stirred and refluxed. Afterwards, potassium carbonate (15.2 g, 110.3 mmol) was dissolved in 46 mL of water, and then added thereto. Thereafter, it was stirred sufficiently, followed by adding bis(tri-tert-butylphosphine) palladium (0) (0.2 g, 0.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-20 of 16.1 g. (Yield 73%, MS: [M+H]⁺=600).

### Synthesis Example 1-21

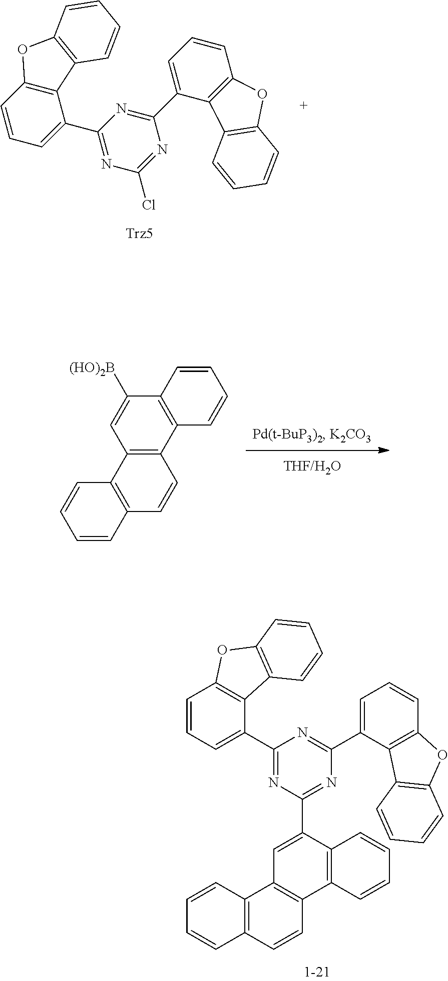 [SMILES: Clc1nc(-c2cccc3oc4ccccc4c23)nc(-c2cccc3oc4ccccc4c23)n1 ; OB(O)c1cc2c3ccccc3ccc2c2ccccc12 ; c1ccc2c(c1)ccc1c3ccccc3c(-c3nc(-c4cccc5oc6ccccc6c45)nc(-c4cccc5oc6ccccc6c45)n3)cc21]

Compound Trz5 (15 g, 33.5 mmol) and chrysen-6-yl boronic acid (9.6 g, 35.2 mmol) were added to 300 mL of tetrahydrofuran (THF), and the mixture was stirred and refluxed. Afterwards, potassium carbonate (13.9 g, 100.5 mmol) was dissolved in 42 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-21 of 16.1 g. (Yield 75%, MS: [M+H]⁺=640).

### Synthesis Example 1-22

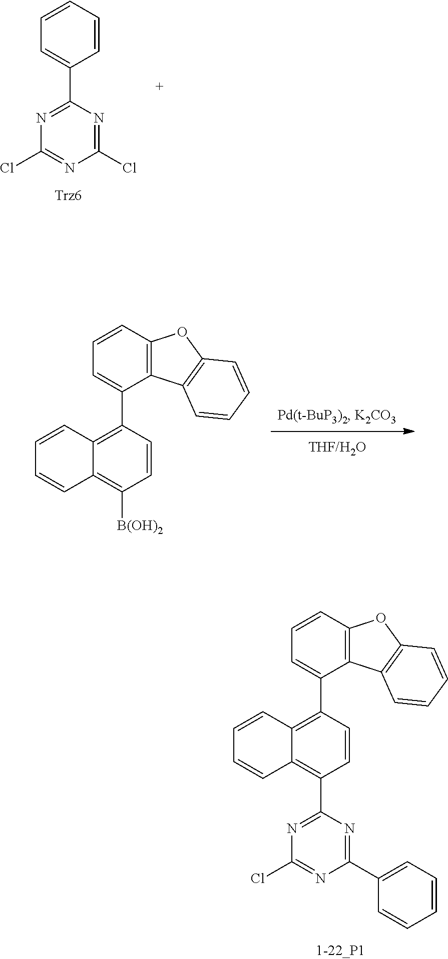 [SMILES: Clc1nc(Cl)nc(-c2ccccc2)n1 ; OB(O)c1ccc(-c2cccc3oc4ccccc4c23)c2ccccc12 ; Clc1nc(-c2ccccc2)nc(-c2ccc(-c3cccc4oc5ccccc5c34)c3ccccc23)n1]

### Step 1) Synthesis of Compound 1-22_P1

Compound Trz6 (15 g, 66.4 mmol) and (4-(dibenzo[b,d]furan-1-yl)naphthalen-1-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 42 mL of water, and then added thereto. Thereafter, it was stirred sufficiently, followed by adding bis(tri-tert-butylphosphine) palladium (0) (0.3 g, 0.7 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-22_P1 of 23.1 g. (Yield 72%, MS: [M+H]⁺=484).

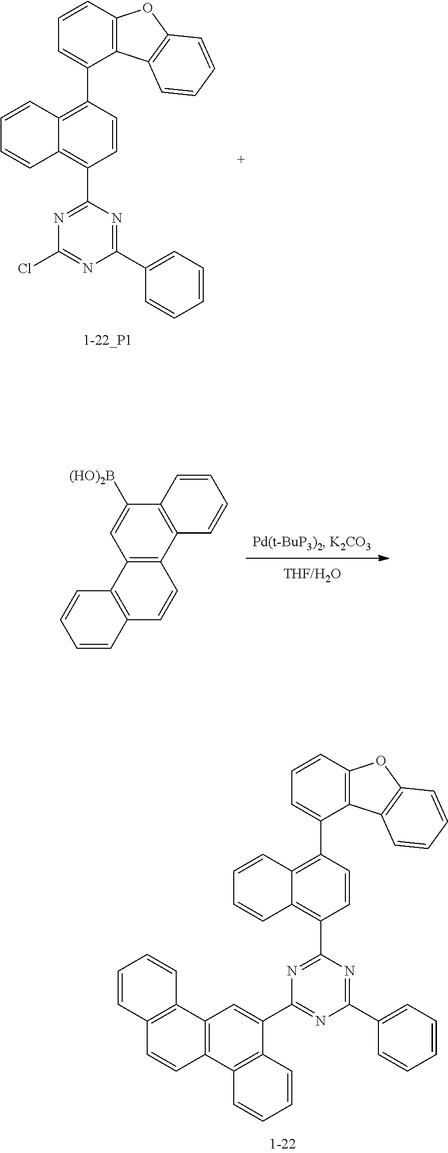 [SMILES: Clc1nc(-c2ccccc2)nc(-c2ccc(-c3cccc4oc5ccccc5c34)c3ccccc23)n1 ; OB(O)c1cc2c3ccccc3ccc2c2ccccc12 ; c1ccc(-c2nc(-c3ccc(-c4cccc5oc6ccccc6c45)c4ccccc34)nc(-c3cc4c5ccccc5ccc4c4ccccc34)n2)cc1]

### Step 2) Synthesis of Compound 1-22

Compound 1-22_P1 (15 g, 31 mmol) prepared in the above Step 1) and chrysen-6-yl boronic acid (8.9 g, 32.5 mmol) were added to 300 mL of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-22 of 14.4 g. (Yield 69%, MS: [M+H]⁺=676).

### Synthesis Example 1-23

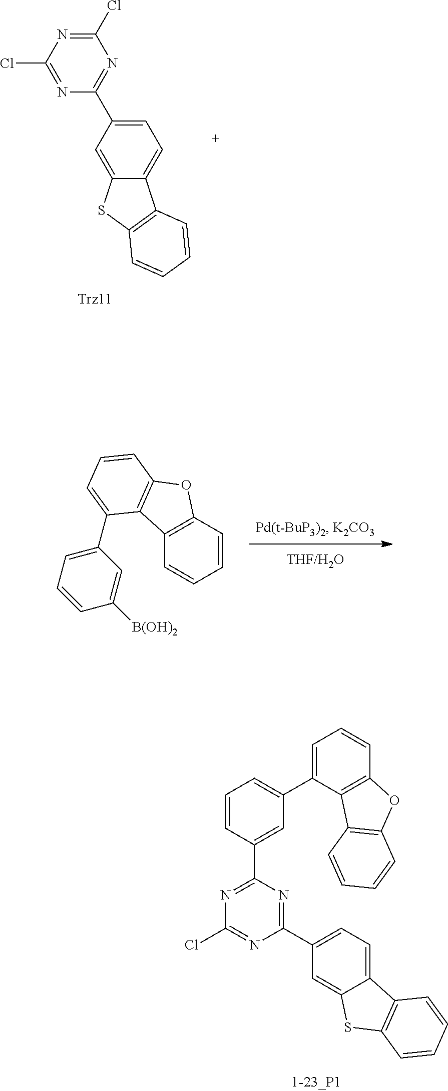 [SMILES: Clc1nc(Cl)nc(-c2ccc3c(c2)sc2ccccc23)n1 ; OB(O)c1cccc(-c2cccc3oc4ccccc4c23)c1 ; Clc1nc(-c2cccc(-c3cccc4oc5ccccc5c34)c2)nc(-c2ccc3c(c2)sc2ccccc23)n1]

### Step 1) Synthesis of Compound 1-23_P1

Compound Trz11 (15 g, 45.2 mmol) and (3-(dibenzo[b,d]furan-1-yl)phenyl) boronic acid (13.7 g, 47.4 mmol) were added to 300 mL of tetrahydrofuran (THF), and the mixture was stirred and refluxed. Afterwards, potassium carbonate (18.7 g, 135.5 mmol) was dissolved in 56 mL of water, and then added thereto. Thereafter, it was stirred sufficiently, followed by adding bis(tri-tert-butylphosphine) palladium (0) (0.2 g, 0.5 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-23_P1 of 17.5 g. (Yield 72%, MS: [M+H]⁺=540).

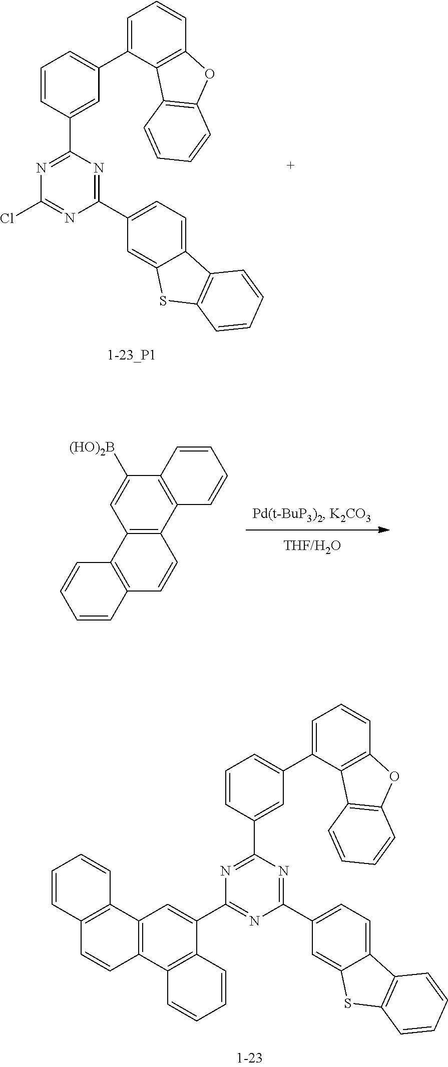 [SMILES: Clc1nc(-c2cccc(-c3cccc4oc5ccccc5c34)c2)nc(-c2ccc3c(c2)sc2ccccc23)n1 ; OB(O)c1cc2c3ccccc3ccc2c2ccccc12 ; c1cc(-c2nc(-c3ccc4c(c3)sc3ccccc34)nc(-c3cc4c5ccccc5ccc4c4ccccc34)n2)cc(-c2cccc3oc4ccccc4c23)c1]

### Step 2) Synthesis of Compound 1-23

Compound 1-23_P1 (15 g, 27.8 mmol) prepared in the above Step 1) and chrysen-6-yl boronic acid (7.9 g, 29.2 mmol) were added to 300 mL of tetrahydrofuran (THF), and the mixture was stirred and refluxed. Afterwards, potassium carbonate (11.5 g, 83.3 mmol) was dissolved in 35 ml of water, and then added thereto. Thereafter, it was stirred sufficiently, followed by adding bis(tri-tert-butylphosphine) palladium (0) (0.1 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-23 of 13.2 g. (Yield 65%, MS: [M+H]⁺=732).

### Synthesis Example 1-24

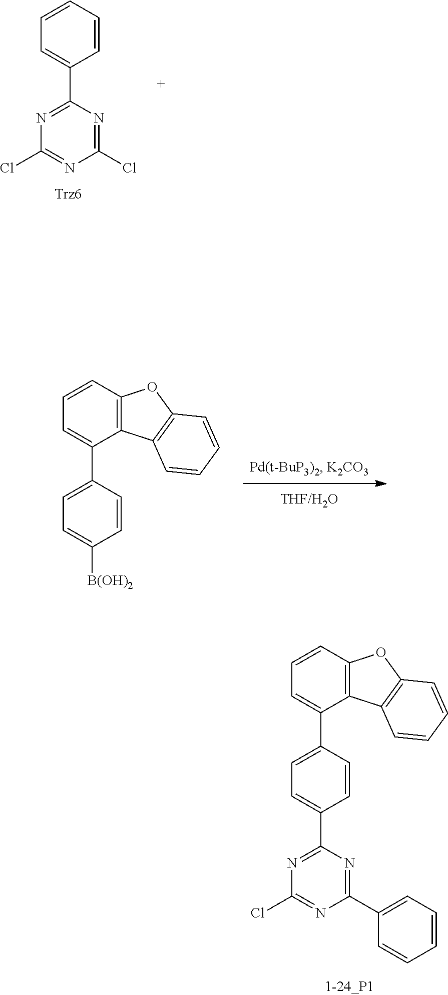 [SMILES: Clc1nc(Cl)nc(-c2ccccc2)n1 ; OB(O)c1ccc(-c2cccc3oc4ccccc4c23)cc1 ; Clc1nc(-c2ccccc2)nc(-c2ccc(-c3cccc4oc5ccccc5c34)cc2)n1]

### Step 1) Synthesis of Compound 1-24_P1

Compound Trz6 (15 g, 66.4 mmol) and (4-(dibenzo[b,d]furan-1-yl)phenyl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-24_P1 of 18.7 g. (Yield 65%, MS: [M+H]⁺=434).

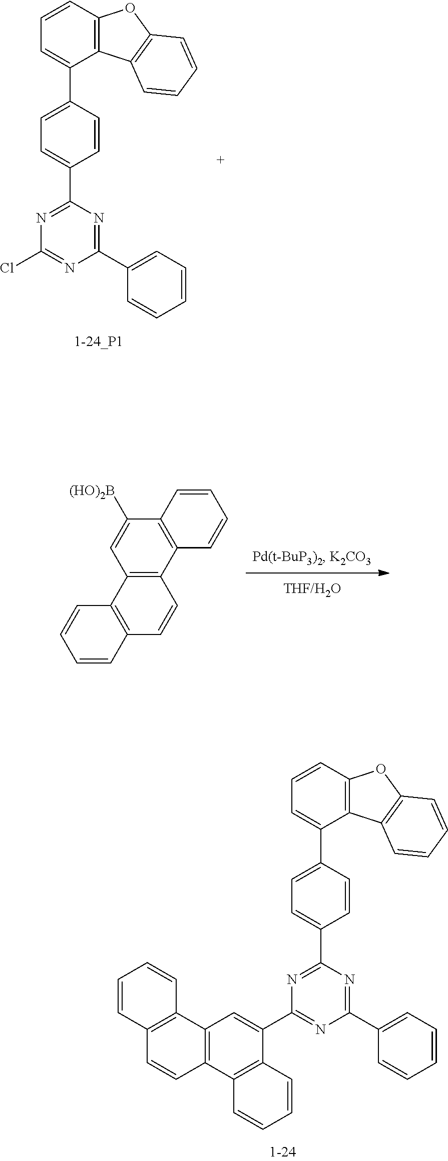 [SMILES: Clc1nc(-c2ccccc2)nc(-c2ccc(-c3cccc4oc5ccccc5c34)cc2)n1 ; OB(O)c1cc2c3ccccc3ccc2c2ccccc12 ; c1ccc(-c2nc(-c3ccc(-c4cccc5oc6ccccc6c45)cc3)nc(-c3cc4c5ccccc5ccc4c4ccccc34)n2)cc1]

### Step 2) Synthesis of Compound 1-24

Compound 1-24_P1 (15 g, 34.6 mmol) prepared in the above Step 1) and chrysen-6-yl boronic acid (9.9 g, 36.3 mmol) were added to 300 mL of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-24 of 15.6 g. (Yield 72%, MS: [M+H]⁺=626).

### Synthesis Example 1-25

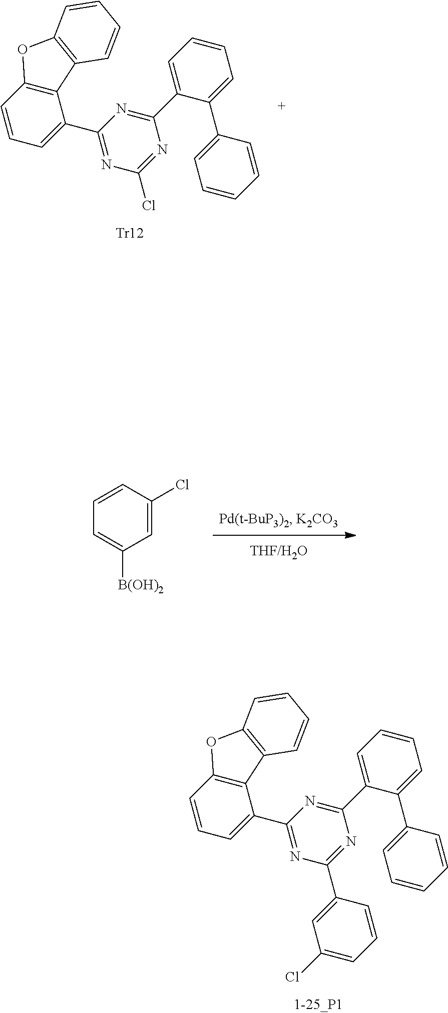 [SMILES: Clc1nc(-c2ccccc2-c2ccccc2)nc(-c2cccc3oc4ccccc4c23)n1 ; OB(O)c1cccc(Cl)c1 ; Clc1cccc(-c2nc(-c3ccccc3-c3ccccc3)nc(-c3cccc4oc5ccccc5c34)n2)c1]

### Step 1) Synthesis of Compound 1-25_P1

Compound Trz12 (15 g, 34.6 mmol) and (3-chlorophenyl) boronic acid (5.7 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-25_P1 of 12.3 g. (Yield 70%, MS: [M+H]⁺=510).

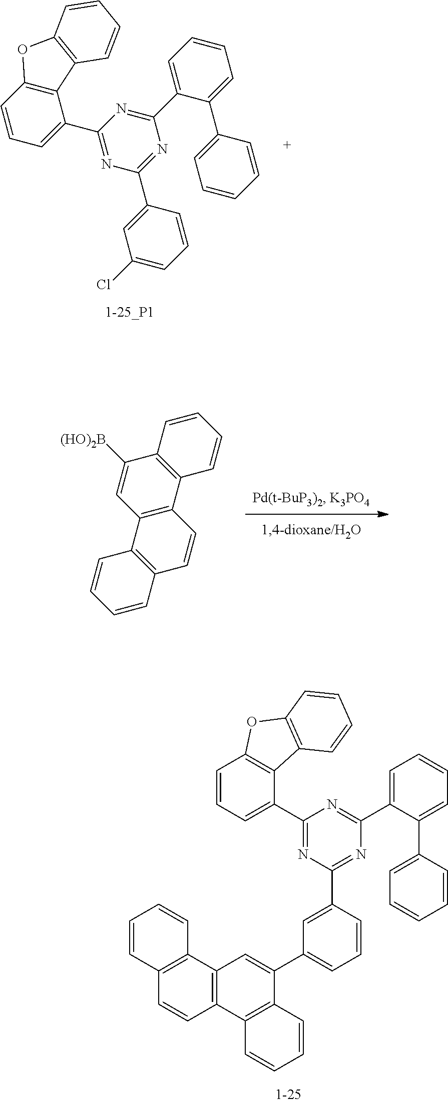 [SMILES: Clc1cccc(-c2nc(-c3ccccc3-c3ccccc3)nc(-c3cccc4oc5ccccc5c34)n2)c1 ; OB(O)c1cc2c3ccccc3ccc2c2ccccc12 ; c1ccc(-c2ccccc2-c2nc(-c3cccc(-c4cc5c6ccccc6ccc5c5ccccc45)c3)nc(-c3cccc4oc5ccccc5c34)n2)cc1]

### Step 2) Synthesis of Compound 1-25

Compound 1-25_P1 (15 g, 29.4 mmol) prepared in the above Step 1) and chrysen-6-yl boronic acid (8.4 g, 30.9 mmol) were added to 300 mL of 1,4-dioxane, and the mixture was stirred and refluxed. Afterwards, potassium phosphate (18.7 g, 88.2 mmol) was dissolved in 56 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-25 of 13.8 g. (Yield 67%, MS: [M+H]⁺=702).

### Synthesis Example 1-26

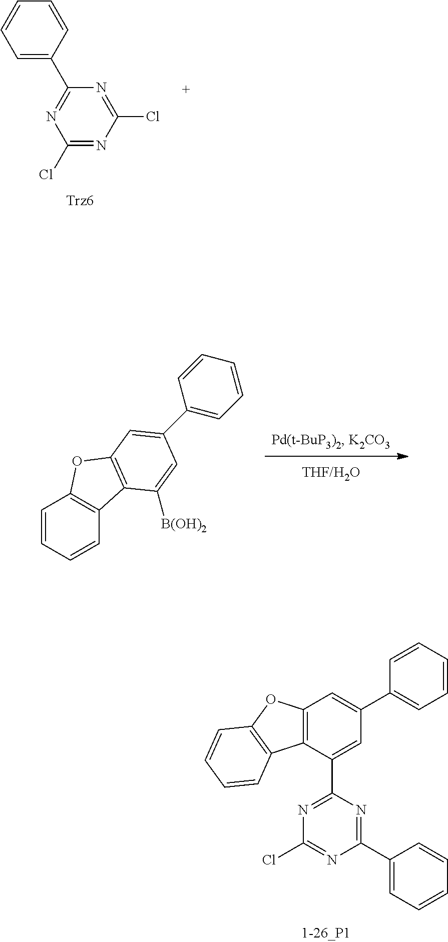 [SMILES: Clc1nc(Cl)nc(-c2ccccc2)n1 ; OB(O)c1cc(-c2ccccc2)cc2oc3ccccc3c12 ; Clc1nc(-c2ccccc2)nc(-c2cc(-c3ccccc3)cc3oc4ccccc4c23)n1]

### Step 1) Synthesis of Compound 1-26_P1

Compound Trz6 (15 g, 66.4 mmol) and (3-phenyldibenzo[b,d]furan-1-yl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-26_P1 of 18.7 g. (Yield 65%, MS: [M+H]⁺=434).

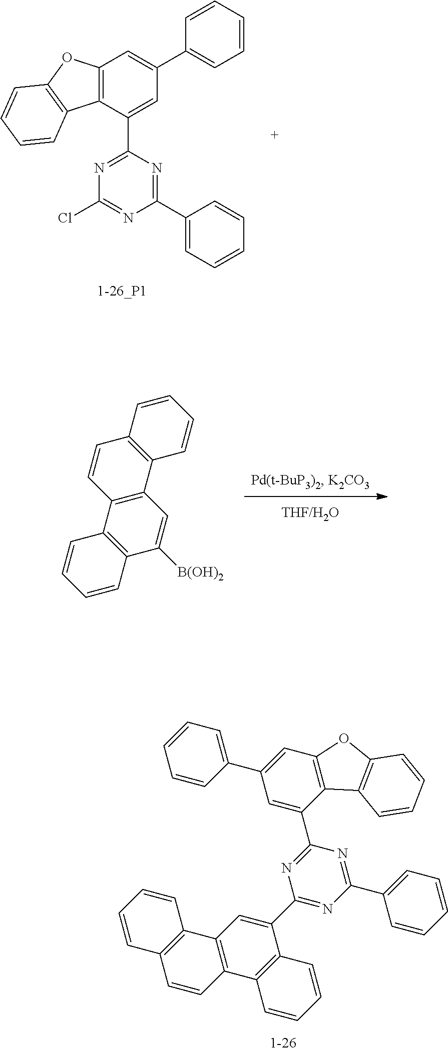 [SMILES: Clc1nc(-c2ccccc2)nc(-c2cc(-c3ccccc3)cc3oc4ccccc4c23)n1 ; OB(O)c1cc2c3ccccc3ccc2c2ccccc12 ; c1ccc(-c2cc(-c3nc(-c4ccccc4)nc(-c4cc5c6ccccc6ccc5c5ccccc45)n3)c3c(c2)oc2ccccc23)cc1]

### Step 2) Synthesis of Compound 1-26

Compound 1-26_P1 (15 g, 34.6 mmol) prepared in the above Step 1) and chrysen-6-yl boronic acid (9.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-26 of 15.3 g. (Yield 71%, MS: [M+H]⁺=626).

### Synthesis Example 1-27

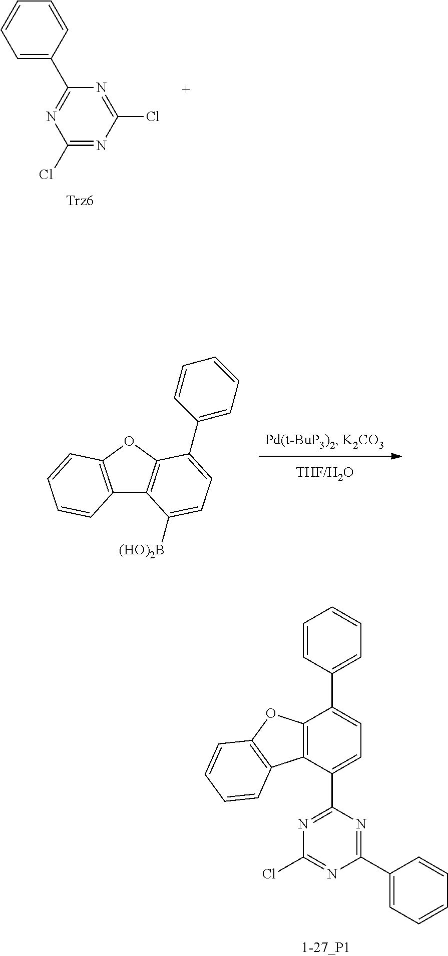 [SMILES: Clc1nc(Cl)nc(-c2ccccc2)n1 ; OB(O)c1ccc(-c2ccccc2)c2oc3ccccc3c12 ; Clc1nc(-c2ccccc2)nc(-c2ccc(-c3ccccc3)c3oc4ccccc4c23)n1]

### Step 1) Synthesis of Compound 1-27_P1

Compound Trz6 (15 g, 66.4 mmol) and (4-phenyldibenzo[b,d]furan-1-yl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-27_P1 of 17.5 g. (Yield 61%, MS: [M+H]⁺=434).

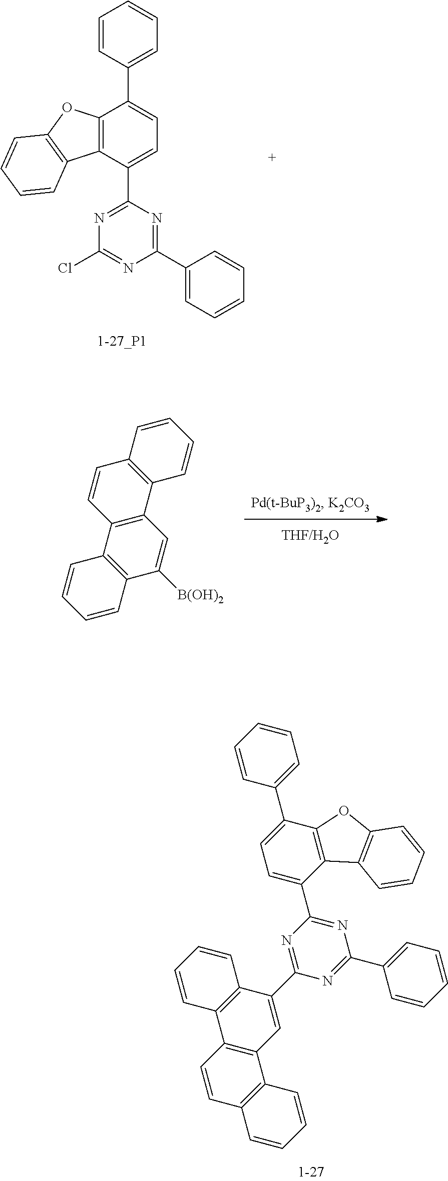 [SMILES: Clc1nc(-c2ccccc2)nc(-c2ccc(-c3ccccc3)c3oc4ccccc4c23)n1 ; OB(O)c1cc2c3ccccc3ccc2c2ccccc12 ; c1ccc(-c2nc(-c3cc4c5ccccc5ccc4c4ccccc34)nc(-c3ccc(-c4ccccc4)c4oc5ccccc5c34)n2)cc1]

### Step 2) Synthesis of Compound 1-27

Compound 1-27_P1 (15 g, 34.6 mmol) prepared in the above Step 1) and chrysen-6-yl boronic acid (9.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-27 of 14.7 g. (Yield 68%, MS: [M+H]⁺=626).

### Synthesis Example 1-28

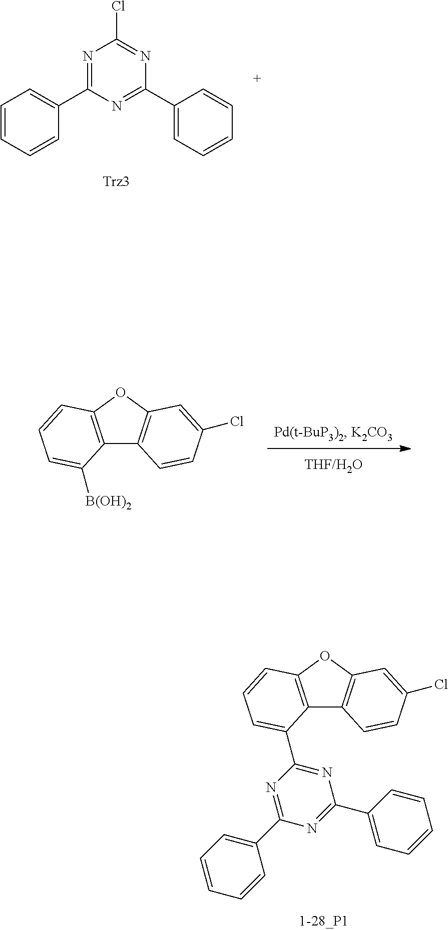 [SMILES: Clc1nc(-c2ccccc2)nc(-c2ccccc2)n1 ; OB(O)c1cccc2oc3cc(Cl)ccc3c12 ; Clc1ccc2c(c1)oc1cccc(-c3nc(-c4ccccc4)nc(-c4ccccc4)n3)c12]

### Step 1) Synthesis of Compound 1-28_P1

Compound Trz3 (15 g, 56 mmol) and (7-chlorodibenzo[b,d]furan-1-yl) boronic acid (14.5 g, 58.8 mmol) were added to 300 mL of tetrahydrofuran (THF), and the mixture was stirred and refluxed. Afterwards, potassium carbonate (23.2 g, 168.1 mmol) was dissolved in 70 mL of water, and then added thereto. Thereafter, it was stirred sufficiently, followed by adding bis(tri-tert-butylphosphine) palladium (0) (0.3 g, 0.6 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-28_P1 of 15 g. (Yield 62%, MS: [M+H]⁺=434).

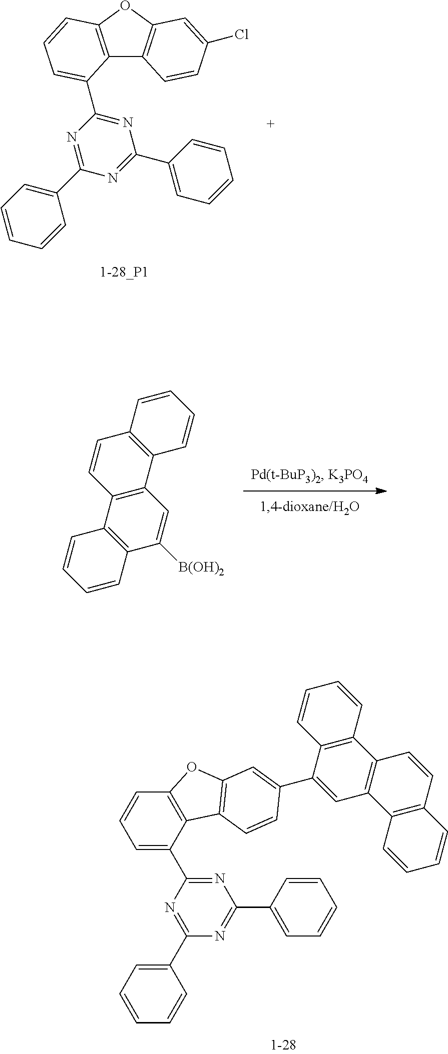 [SMILES: Clc1ccc2c(c1)oc1cccc(-c3nc(-c4ccccc4)nc(-c4ccccc4)n3)c12 ; OB(O)c1cc2c3ccccc3ccc2c2ccccc12 ; c1ccc(-c2nc(-c3ccccc3)nc(-c3cccc4oc5cc(-c6cc7c8ccccc8ccc7c7ccccc67)ccc5c34)n2)cc1]

### Step 2) Synthesis of Compound 1-28

Compound 1-28_P1 (15 g, 34.6 mmol) prepared in the above Step 1) and chrysen-6-yl boronic acid (9.9 g, 36.3 mmol) were added to 300 mL of 1,4-dioxane, and the mixture was stirred and refluxed. Afterwards, potassium phosphate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-28 of 15.3 g. (Yield 71%, MS: [M+H]⁺=626).

### Synthesis Example 1-29

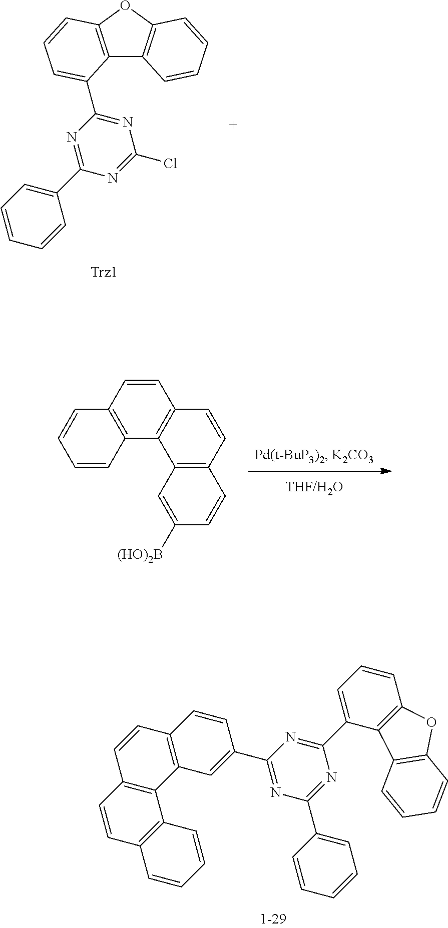 [SMILES: Clc1nc(-c2ccccc2)nc(-c2cccc3oc4ccccc4c23)n1 ; OB(O)c1ccc2ccc3ccc4ccccc4c3c2c1 ; c1ccc(-c2nc(-c3ccc4ccc5ccc6ccccc6c5c4c3)nc(-c3cccc4oc5ccccc5c34)n2)cc1]

Compound Trz1 (15 g, 41.9 mmol) and benzo[c]phenanthren-2-yl boronic acid (12 g, 44 mmol) were added to 300 mL of tetrahydrofuran (THF), and the mixture was stirred and refluxed. Afterwards, potassium carbonate (17.4 g, 125.7 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-29 of 16.3 g. (Yield 71%, MS: [M+H]⁺=550).

### Synthesis Example 1-30

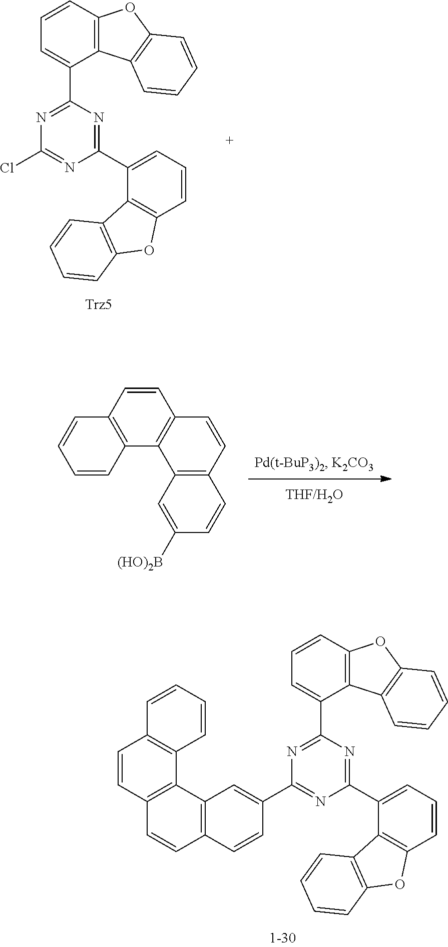 [SMILES: Clc1nc(-c2cccc3oc4ccccc4c23)nc(-c2cccc3oc4ccccc4c23)n1 ; OB(O)c1ccc2ccc3ccc4ccccc4c3c2c1 ; c1ccc2c(c1)ccc1ccc3ccc(-c4nc(-c5cccc6oc7ccccc7c56)nc(-c5cccc6oc7ccccc7c56)n4)cc3c12]

Compound Trz5 (15 g, 33.5 mmol) and benzo[c]phenanthren-2-yl boronic acid (9.6 g, 35.2 mmol) were added to 300 ml of tetrahydrofuran (THF), and the mixture was stirred and refluxed. Afterwards, potassium carbonate (13.9 g, 100.5 mmol) was dissolved in 42 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-30 of 16.1 g. (Yield 75%, MS: [M+H]⁺=640).

### Synthesis Example 1-31

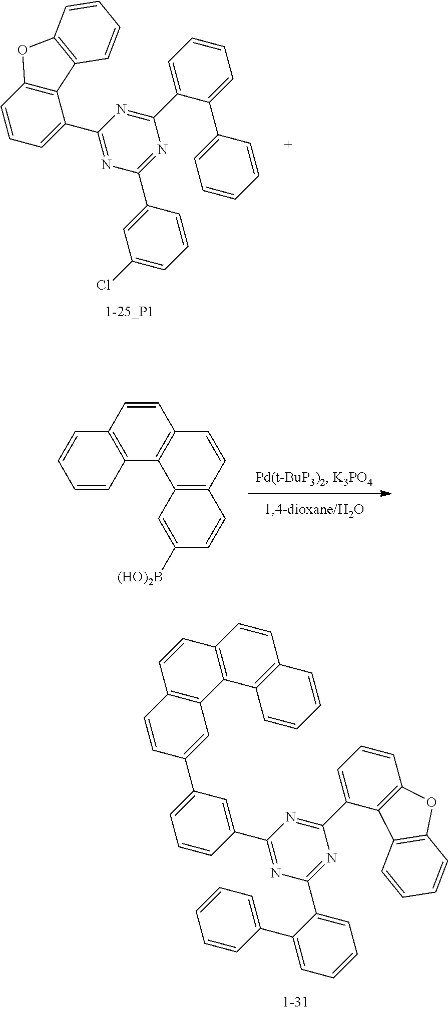 [SMILES: Clc1cccc(-c2nc(-c3ccccc3-c3ccccc3)nc(-c3cccc4oc5ccccc5c34)n2)c1 ; OB(O)c1ccc2ccc3ccc4ccccc4c3c2c1 ; c1ccc(-c2ccccc2-c2nc(-c3cccc(-c4ccc5ccc6ccc7ccccc7c6c5c4)c3)nc(-c3cccc4oc5ccccc5c34)n2)cc1]

Compound 1-25_P1 (15 g, 29.4 mmol) and benzo[c]phenanthren-2-yl boronic acid (8.4 g, 30.9 mmol) were added to 300 mL of 1,4-dioxane, and the mixture was stirred and refluxed. Afterwards, potassium phosphate (18.7 g, 88.2 mmol) was dissolved in 56 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-31 of 13 g. (Yield 63%, MS: [M+H]⁺=702).

### Synthesis Example 1-32

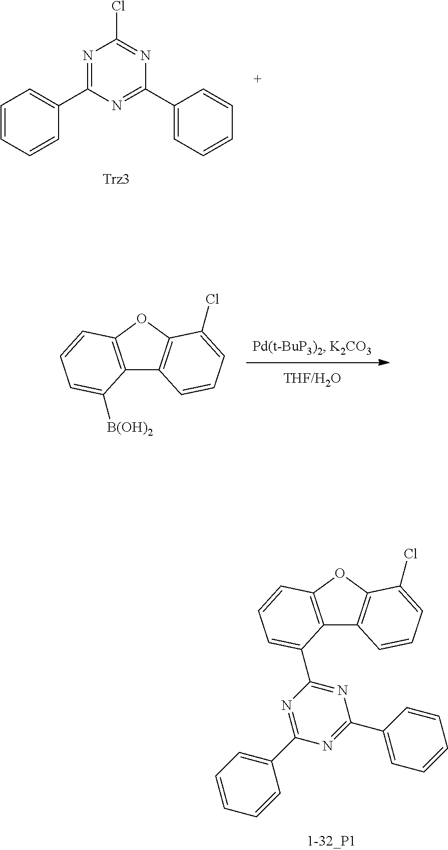 [SMILES: Clc1nc(-c2ccccc2)nc(-c2ccccc2)n1 ; OB(O)c1cccc2oc3c(Cl)cccc3c12 ; Clc1cccc2c1oc1cccc(-c3nc(-c4ccccc4)nc(-c4ccccc4)n3)c12]

### Step 1) Synthesis of Compound 1-32_P1

Compound Trz3 (15 g, 56 mmol) and (6-chlorodibenzo[b,d]furan-1-yl) boronic acid (14.5 g, 58.8 mmol) were added to 300 mL of tetrahydrofuran (THF), and the mixture was stirred and refluxed. Afterwards, potassium carbonate (23.2 g, 168.1 mmol) was dissolved in 70 mL of water, and then added thereto. Thereafter, it was stirred sufficiently, followed by adding bis(tri-tert-butylphosphine) palladium (0) (0.3 g, 0.6 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-32_P1 of 15.5 g. (Yield 64%, MS: [M+H]⁺=434).

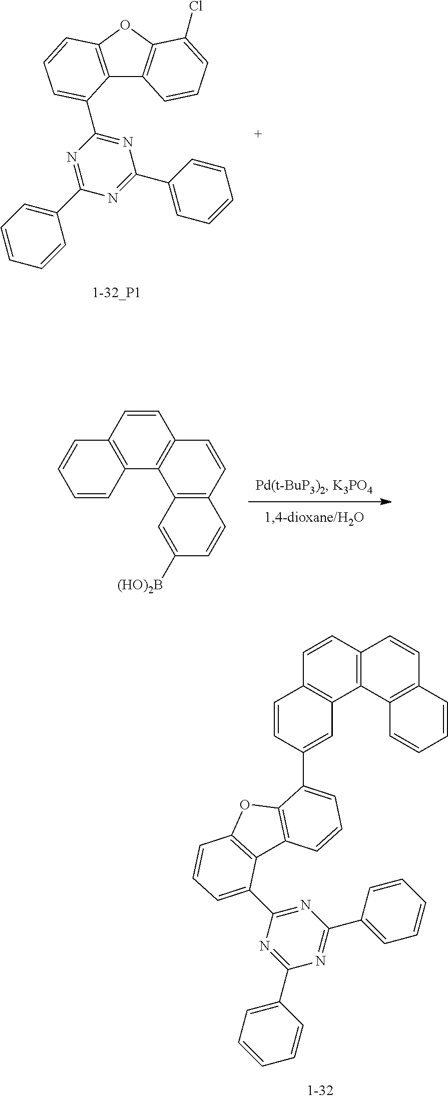 [SMILES: Clc1cccc2c1oc1cccc(-c3nc(-c4ccccc4)nc(-c4ccccc4)n3)c12 ; OB(O)c1ccc2ccc3ccc4ccccc4c3c2c1 ; c1ccc(-c2nc(-c3ccccc3)nc(-c3cccc4oc5c(-c6ccc7ccc8ccc9ccccc9c8c7c6)cccc5c34)n2)cc1]

### Step 2) Synthesis of Compound 1-32

Compound 1-32_P1 (15 g, 28.1 mmol) prepared in the above Step 1) and benzo[c]phenanthren-2-yl boronic acid (8 g, 29.5 mmol) were added to 300 ml of 1,4-dioxane, and the mixture was stirred and refluxed. Afterwards, potassium phosphate (17.9 g, 84.3 mmol) was dissolved in 54 mL of water, and then added thereto. Thereafter, it was stirred sufficiently, followed by adding bis(tri-tert-butylphosphine) palladium (0) (0.1 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-32 of 10.5 g. (Yield 60%, MS: [M+H]⁺=626).

### Synthesis Example 1-33

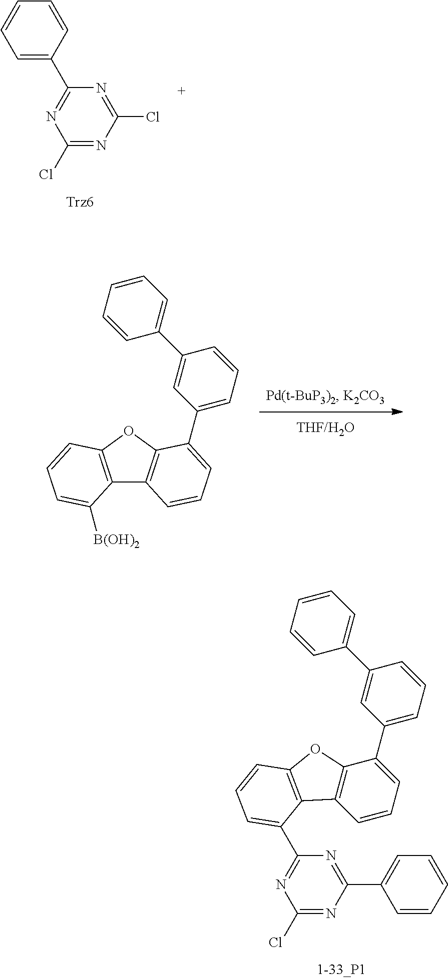 [SMILES: Clc1nc(Cl)nc(-c2ccccc2)n1 ; OB(O)c1cccc2oc3c(-c4cccc(-c5ccccc5)c4)cccc3c12 ; Clc1nc(-c2ccccc2)nc(-c2cccc3oc4c(-c5cccc(-c6ccccc6)c5)cccc4c23)n1]

### Step 1) Synthesis of Compound 1-33_P1

Compound Trz6 (15 g, 66.4 mmol) and (6-([1,1′-biphenyl]-3-yl)dibenzo[b,d]furan-1-yl) boronic acid (24.1 g, 69.7 mmol) were added to 300 ml of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-33_P1 of 22.3 g. (Yield 66%, MS: [M+H]⁺=510).

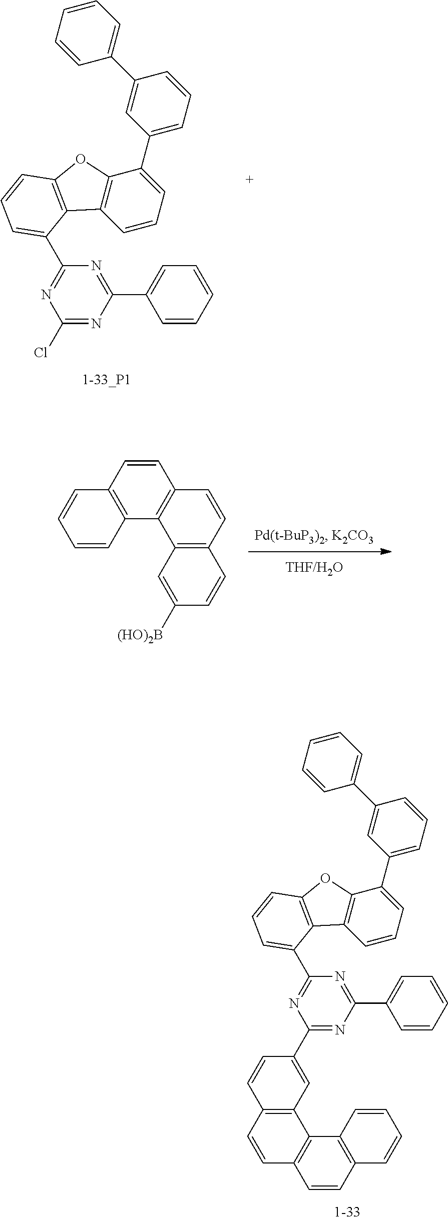 [SMILES: Clc1nc(-c2ccccc2)nc(-c2cccc3oc4c(-c5cccc(-c6ccccc6)c5)cccc4c23)n1 ; OB(O)c1ccc2ccc3ccc4ccccc4c3c2c1 ; c1ccc(-c2cccc(-c3cccc4c3oc3cccc(-c5nc(-c6ccccc6)nc(-c6ccc7ccc8ccc9ccccc9c8c7c6)n5)c34)c2)cc1]

### Step 2) Synthesis of Compound 1-33

Compound 1-33_P1 (15 g, 29.4 mmol) prepared in the above Step 1) and benzo[c]phenanthren-2-yl boronic acid (8.4 g, 30.9 mmol) were added to 300 mL of tetrahydrofuran (THF), and the mixture was stirred and refluxed. Afterwards, potassium carbonate (12.2 g, 88.2 mmol) was dissolved in 37 ml of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-33 of 14.2 g. (Yield 69%, MS: [M+H]⁺=702).

### Synthesis Example 1-34

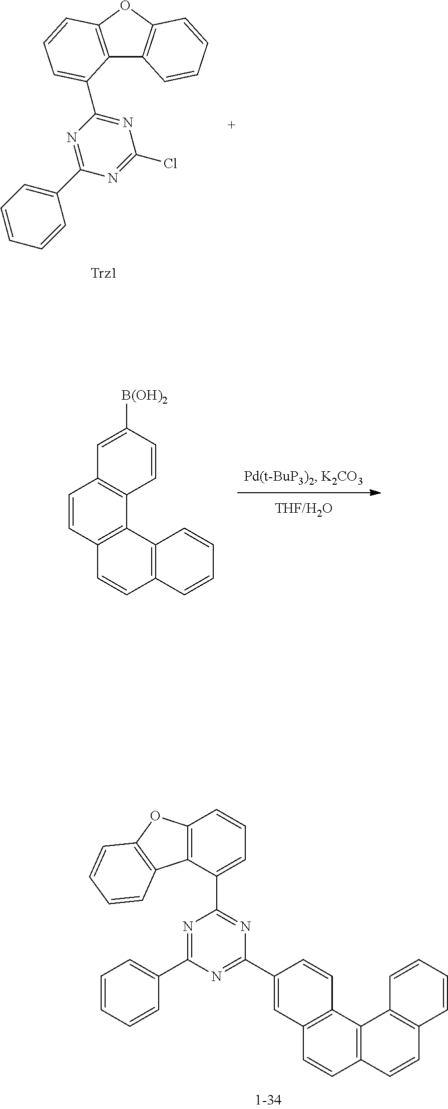 [SMILES: Clc1nc(-c2ccccc2)nc(-c2cccc3oc4ccccc4c23)n1 ; OB(O)c1ccc2c(ccc3ccc4ccccc4c32)c1 ; c1ccc(-c2nc(-c3ccc4c(ccc5ccc6ccccc6c54)c3)nc(-c3cccc4oc5ccccc5c34)n2)cc1]

Compound Trz1 (15 g, 41.9 mmol) and benzo[c]phenanthren-3-yl boronic acid (12 g, 44 mmol) were added to 300 mL of tetrahydrofuran (THF), and the mixture was stirred and refluxed. Afterwards, potassium carbonate (17.4 g, 125.7 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-34 of 16.1 g. (Yield 70%, MS: [M+H]⁺=550).

### Synthesis Example 1-35

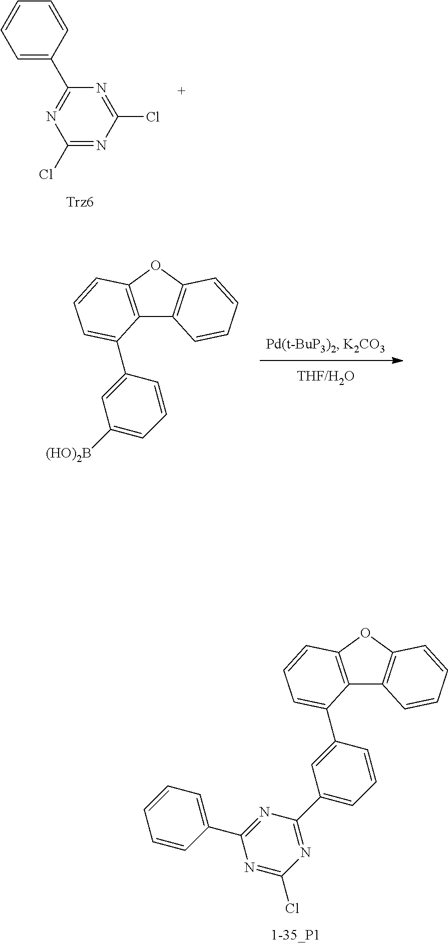 [SMILES: Clc1nc(Cl)nc(-c2ccccc2)n1 ; OB(O)c1cccc(-c2cccc3oc4ccccc4c23)c1 ; Clc1nc(-c2ccccc2)nc(-c2cccc(-c3cccc4oc5ccccc5c34)c2)n1]

### Step 1) Synthesis of Compound 1-35_P1

Compound Trz6 (15 g, 66.4 mmol) and (3-(dibenzo[b,d]furan-1-yl)phenyl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-35_P1 of 20.4 g. (Yield 71%, MS: [M+H]⁺=434).

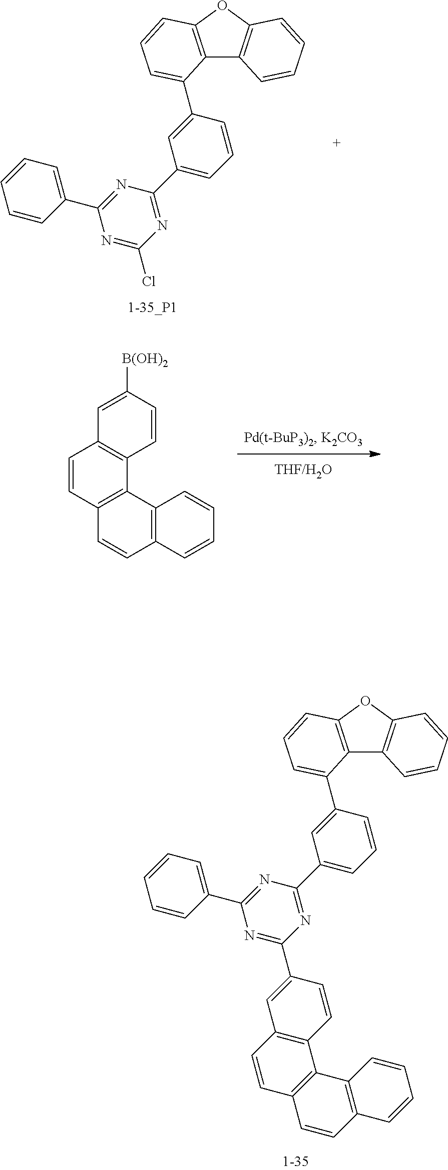 [SMILES: Clc1nc(-c2ccccc2)nc(-c2cccc(-c3cccc4oc5ccccc5c34)c2)n1 ; OB(O)c1ccc2c(ccc3ccc4ccccc4c32)c1 ; c1ccc(-c2nc(-c3cccc(-c4cccc5oc6ccccc6c45)c3)nc(-c3ccc4c(ccc5ccc6ccccc6c54)c3)n2)cc1]

### Step 2) Synthesis of Compound 1-35

Compound 1-35_P1 (15 g, 34.6 mmol) prepared in the above Step 1) and benzo[c]phenanthren-3-yl boronic acid (9.9 g, 36.3 mmol) were added to 300 ml of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-35 of 15.8 g. (Yield 73%, MS: [M+H]⁺=626).

### Synthesis Example 1-36

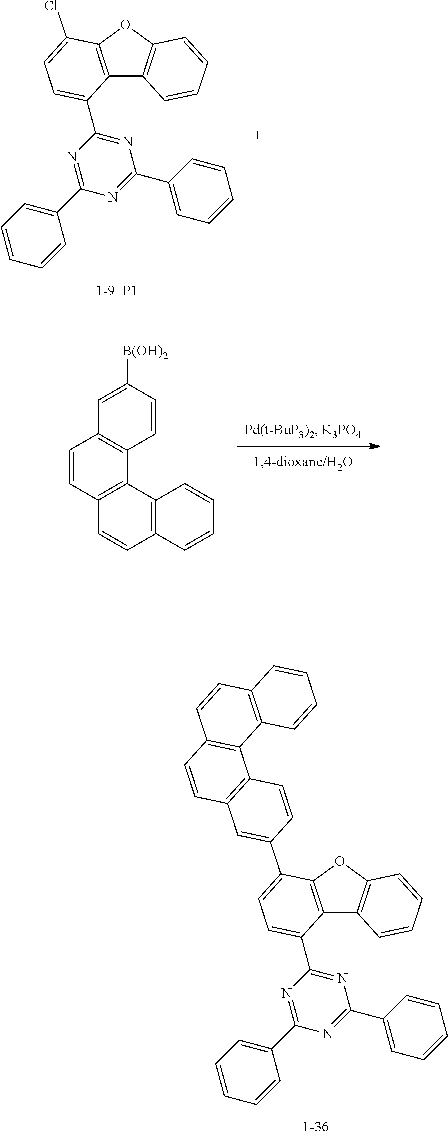 [SMILES: Clc1ccc(-c2nc(-c3ccccc3)nc(-c3ccccc3)n2)c2c1oc1ccccc12 ; OB(O)c1ccc2c(ccc3ccc4ccccc4c32)c1 ; c1ccc(-c2nc(-c3ccccc3)nc(-c3ccc(-c4ccc5c(ccc6ccc7ccccc7c65)c4)c4oc5ccccc5c34)n2)cc1]

Compound 1-9_P1 (15 g, 34.6 mmol) and benzo[c]phenanthren-3-yl boronic acid (9.9 g, 36.3 mmol) were added to 300 mL of 1,4-dioxane, and the mixture was stirred and refluxed. Afterwards, potassium phosphate (22 g, 103.7 mmol) was dissolved in 66 mL of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-36 of 16.2 g. (Yield 75%, MS: [M+H]⁺=626).

### Synthesis Example 1-37

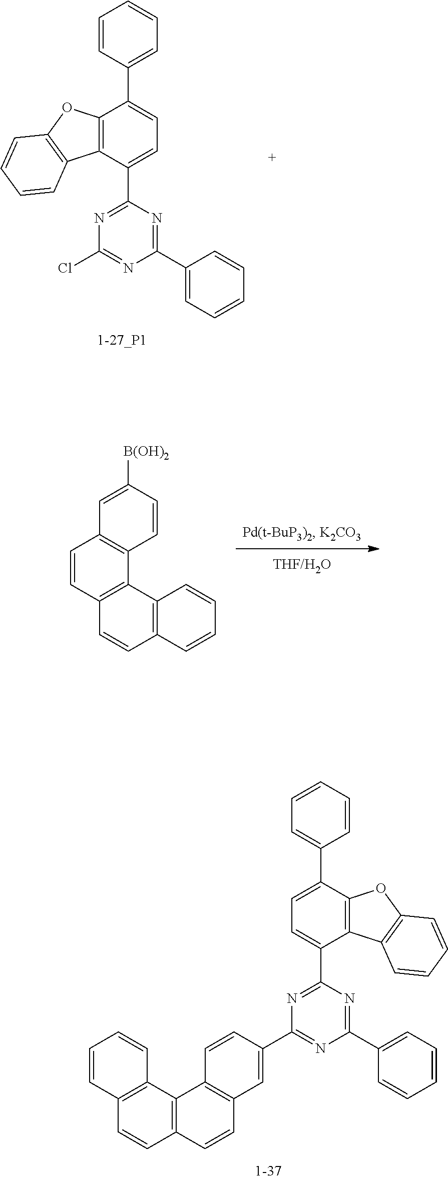 [SMILES: Clc1nc(-c2ccccc2)nc(-c2ccc(-c3ccccc3)c3oc4ccccc4c23)n1 ; OB(O)c1ccc2c(ccc3ccc4ccccc4c32)c1 ; c1ccc(-c2nc(-c3ccc4c(ccc5ccc6ccccc6c54)c3)nc(-c3ccc(-c4ccccc4)c4oc5ccccc5c34)n2)cc1]

Compound 1-27_P1 (15 g, 34.6 mmol) and benzo[c]phenanthren-3-yl boronic acid (9.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-37 of 14.7 g. (Yield 68%, MS: [M+H]⁺=626).

### Synthesis Example 1-38

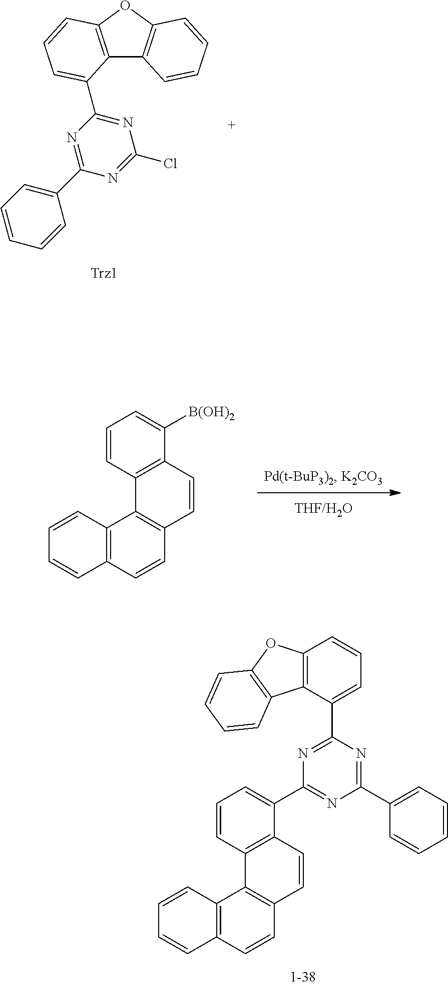 [SMILES: Clc1nc(-c2ccccc2)nc(-c2cccc3oc4ccccc4c23)n1 ; OB(O)c1cccc2c1ccc1ccc3ccccc3c12 ; c1ccc(-c2nc(-c3cccc4c3ccc3ccc5ccccc5c34)nc(-c3cccc4oc5ccccc5c34)n2)cc1]

Compound Trz1 (15 g, 41.9 mmol) and benzo[c]phenanthren-4-yl boronic acid (12 g, 44 mmol) were added to 300 mL of tetrahydrofuran (THF), and the mixture was stirred and refluxed. Afterwards, potassium carbonate (17.4 g, 125.8 mmol) was dissolved in 52 ml of water, and then added thereto. Thereafter, it was stirred sufficiently, followed by adding bis(tri-tert-butylphosphine) palladium (0) (0.2 g, 0.4 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-38 of 15.2 g. (Yield 66%, MS: [M+H]⁺=550).

### Synthesis Example 1-39

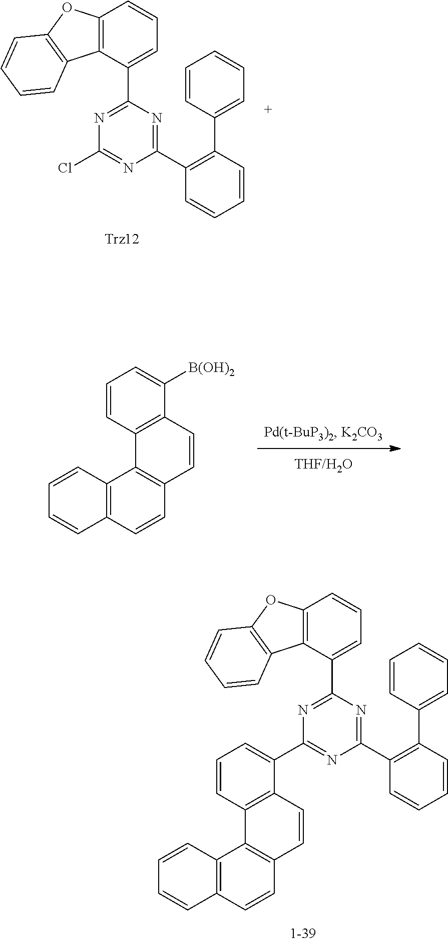 [SMILES: Clc1nc(-c2ccccc2-c2ccccc2)nc(-c2cccc3oc4ccccc4c23)n1 ; OB(O)c1cccc2c1ccc1ccc3ccccc3c12 ; c1ccc(-c2ccccc2-c2nc(-c3cccc4c3ccc3ccc5ccccc5c34)nc(-c3cccc4oc5ccccc5c34)n2)cc1]

Compound Trz12 (15 g, 34.6 mmol) and benzo[c]phenanthren-4-yl boronic acid (9.9 g, 36.3 mmol) were added to 300 ml of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-39 of 14.9 g. (Yield 69%, MS: [M+H]⁺=626).

### Synthesis Example 1-40

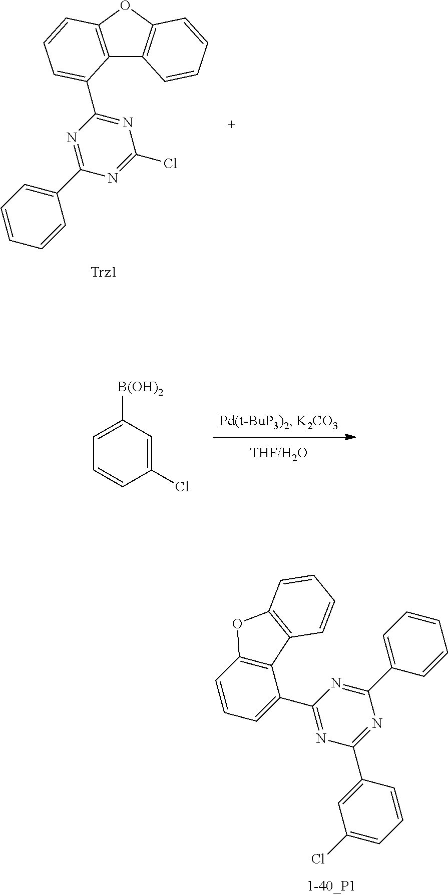 [SMILES: Clc1nc(-c2ccccc2)nc(-c2cccc3oc4ccccc4c23)n1 ; OB(O)c1cccc(Cl)c1 ; Clc1cccc(-c2nc(-c3ccccc3)nc(-c3cccc4oc5ccccc5c34)n2)c1]

### Step 1) Synthesis of Compound 1-40_P1

Compound Trz1 (15 g, 41.9 mmol) and (3-chlorophenyl) boronic acid (6.9 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-40_P1 of 11.4 g. (Yield 63%, MS:

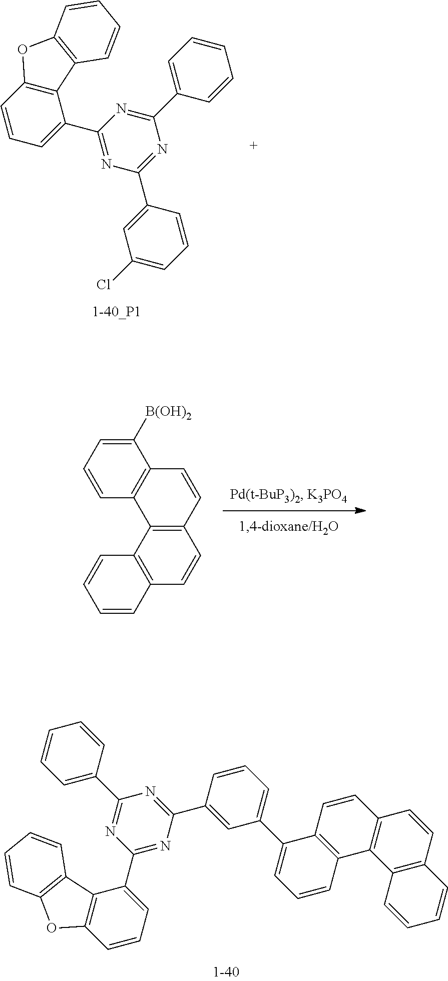 [SMILES: Clc1cccc(-c2nc(-c3ccccc3)nc(-c3cccc4oc5ccccc5c34)n2)c1 ; OB(O)c1cccc2c1ccc1ccc3ccccc3c12 ; c1ccc(-c2nc(-c3cccc(-c4cccc5c4ccc4ccc6ccccc6c45)c3)nc(-c3cccc4oc5ccccc5c34)n2)cc1]

### Step 2) Synthesis of Compound 1-40

Compound 1-40_P1 (15 g, 34.6 mmol) prepared in the above Step 1) and benzo[c]phenanthren-4-yl boronic acid (9.9 g, 36.3 mmol) were added to 300 mL of 1,4-dioxane, and the mixture was stirred and refluxed. Afterwards, potassium phosphate (22 g, 103.7 mmol) was dissolved in 66 ml of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-40 of 13.4 g. (Yield 62%, MS: [M+H]⁺=626).

### Synthesis Example 1-41

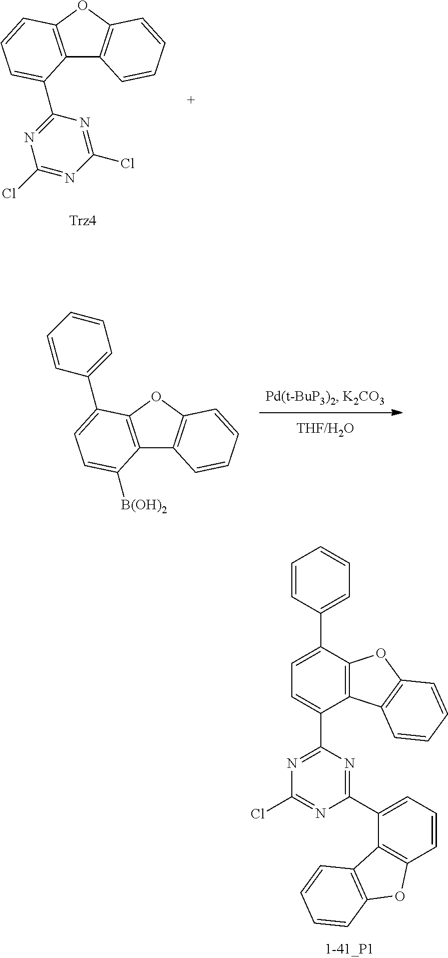 [SMILES: Clc1nc(Cl)nc(-c2cccc3oc4ccccc4c23)n1 ; OB(O)c1ccc(-c2ccccc2)c2oc3ccccc3c12 ; Clc1nc(-c2cccc3oc4ccccc4c23)nc(-c2ccc(-c3ccccc3)c3oc4ccccc4c23)n1]

### Step 1) Synthesis of Compound 1-41_P1

Compound Trz4 (15 g, 47.4 mmol) and (4-phenyldibenzo[b,d]furan-1-yl) boronic acid (14.4 g, 49.8 mmol) were added to 300 mL of tetrahydrofuran (THF), and the mixture was stirred and refluxed. Afterwards, potassium carbonate (19.7 g, 142.3 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-41_P1 of 17.1 g. (Yield 69%, MS: [M+H]⁺=524).

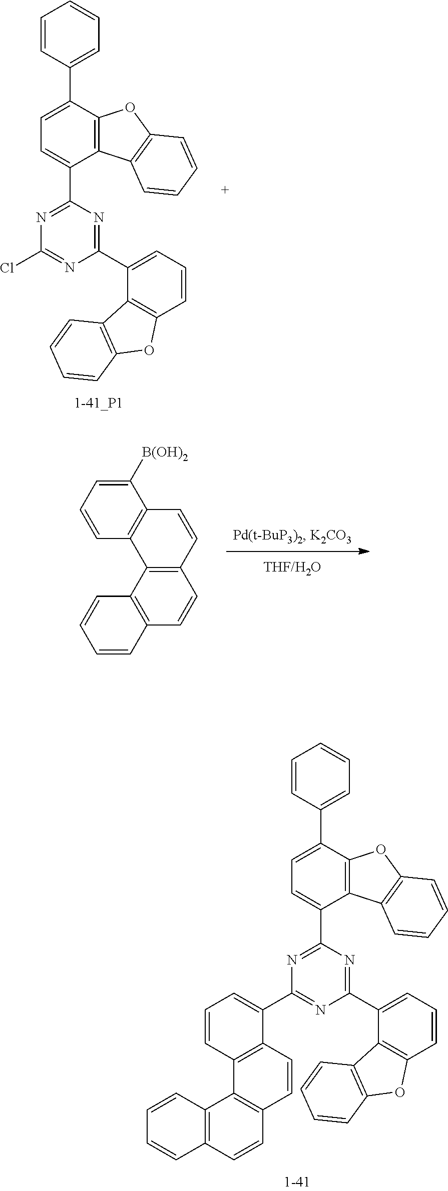 [SMILES: Clc1nc(-c2cccc3oc4ccccc4c23)nc(-c2ccc(-c3ccccc3)c3oc4ccccc4c23)n1 ; OB(O)c1cccc2c1ccc1ccc3ccccc3c12 ; c1ccc(-c2ccc(-c3nc(-c4cccc5c4ccc4ccc6ccccc6c45)nc(-c4cccc5oc6ccccc6c45)n3)c3c2oc2ccccc23)cc1]

### Step 2) Synthesis of Compound 1-41

Compound 1-41_P1 (15 g, 28.6 mmol) prepared in the above Step 1) and benzo[c]phenanthren-4-yl boronic acid (8.2 g, 30.1 mmol) were added to 300 ml of tetrahydrofuran (THF), and the mixture was stirred and refluxed. Afterwards, potassium carbonate (11.9 g, 85.9 mmol) was dissolved in 36 mL of water, and then added thereto. Thereafter, it was stirred sufficiently, followed by adding bis(tri-tert-butylphosphine) palladium (0) (0.1 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-41 of 12.9 g. (Yield 63%, MS: [M+H]⁺=716).

### Synthesis Example 1-42

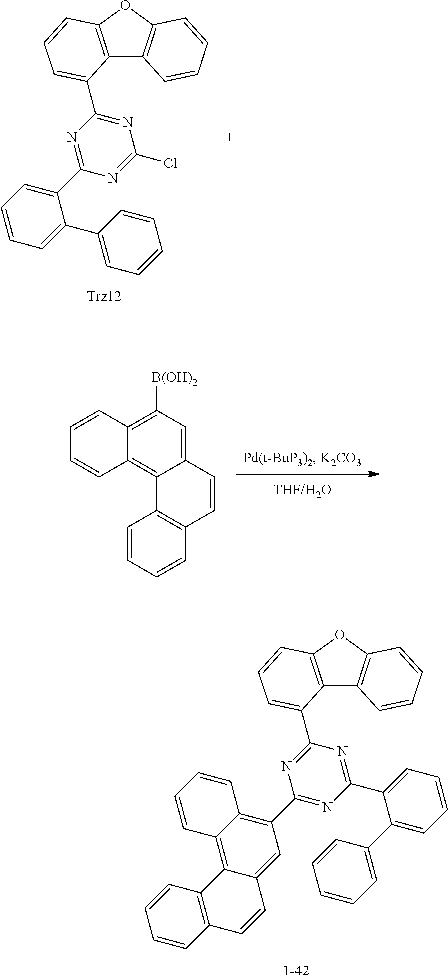 [SMILES: Clc1nc(-c2ccccc2-c2ccccc2)nc(-c2cccc3oc4ccccc4c23)n1 ; OB(O)c1cc2ccc3ccccc3c2c2ccccc12 ; c1ccc(-c2ccccc2-c2nc(-c3cc4ccc5ccccc5c4c4ccccc34)nc(-c3cccc4oc5ccccc5c34)n2)cc1]

Compound Trz12 (15 g, 34.6 mmol) and benzo[c]phenanthren-5-yl boronic acid (9.9 g, 36.3 mmol) were added to 300 ml of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-42 of 13.4 g. (Yield 62%, MS: [M+H]⁺=626).

### Synthesis Example 1-43

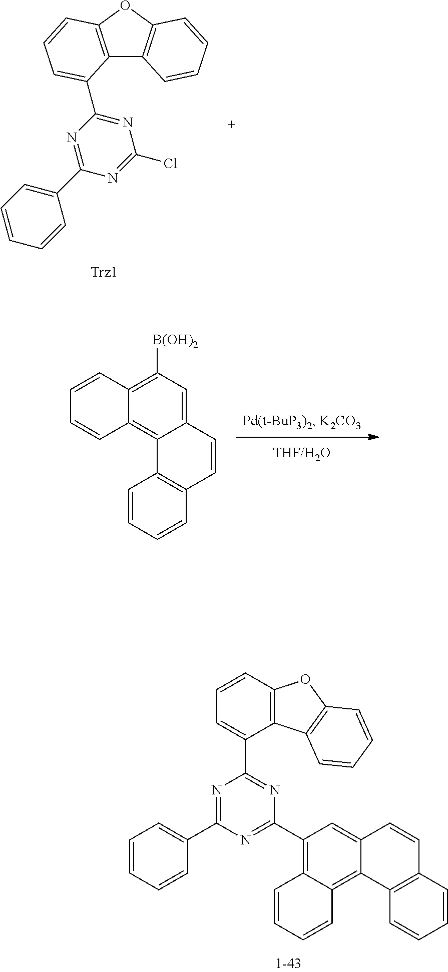 [SMILES: Clc1nc(-c2ccccc2)nc(-c2cccc3oc4ccccc4c23)n1 ; OB(O)c1cc2ccc3ccccc3c2c2ccccc12 ; c1ccc(-c2nc(-c3cc4ccc5ccccc5c4c4ccccc34)nc(-c3cccc4oc5ccccc5c34)n2)cc1]

Compound Trz1 (15 g, 41.9 mmol) and benzo[c]phenanthren-5-yl boronic acid (12 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-43 of 15.4 g. (Yield 67%, MS: [M+H]⁺=550).

### Synthesis Example 1-44

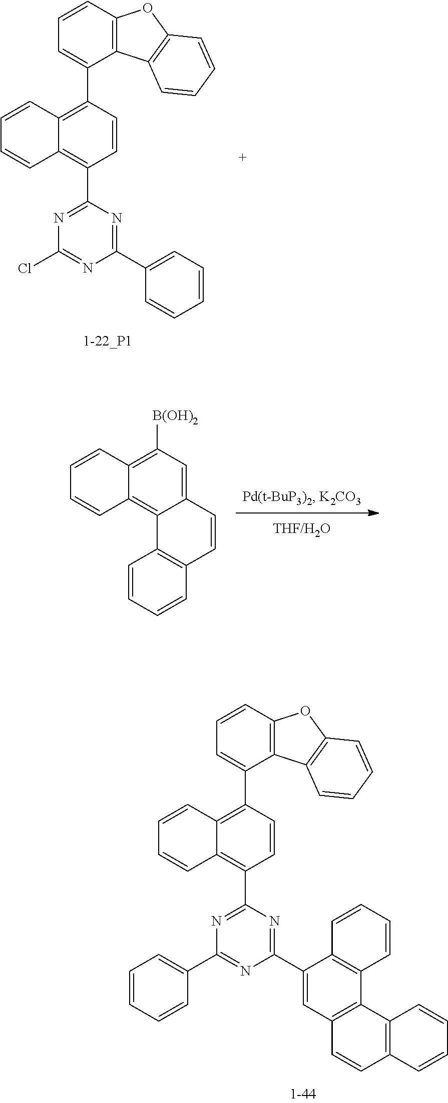 [SMILES: Clc1nc(-c2ccccc2)nc(-c2ccc(-c3cccc4oc5ccccc5c34)c3ccccc23)n1 ; OB(O)c1cc2ccc3ccccc3c2c2ccccc12 ; c1ccc(-c2nc(-c3ccc(-c4cccc5oc6ccccc6c45)c4ccccc34)nc(-c3cc4ccc5ccccc5c4c4ccccc34)n2)cc1]

Compound 1-22_P1 (15 g, 31 mmol) and benzo[c]phenanthren-5-yl boronic acid (8.9 g, 32.5 mmol) were added to 300 ml of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.4 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-44 of 15.1 g. (Yield 72%, MS: [M+H]⁺=676).

### Synthesis Example 1-45

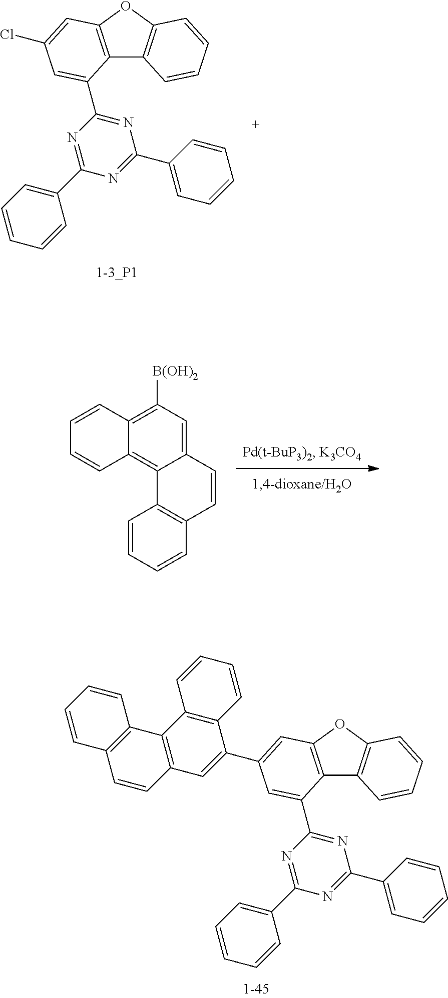 [SMILES: Clc1cc(-c2nc(-c3ccccc3)nc(-c3ccccc3)n2)c2c(c1)oc1ccccc12 ; OB(O)c1cc2ccc3ccccc3c2c2ccccc12 ; c1ccc(-c2nc(-c3ccccc3)nc(-c3cc(-c4cc5ccc6ccccc6c5c5ccccc45)cc4oc5ccccc5c34)n2)cc1]

Compound 1-3_P1 (15 g, 34.6 mmol) and benzo[c]phenanthren-5-yl boronic acid (9.9 g, 36.3 mmol) were added to 300 mL of 1,4-dioxane, and the mixture was stirred and refluxed. Afterwards, potassium phosphate (22 g, 103.7 mmol) was dissolved in 66 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-45 of 13.2 g. (Yield 61%, MS: [M+H]⁺=626).

### Synthesis Example 1-46

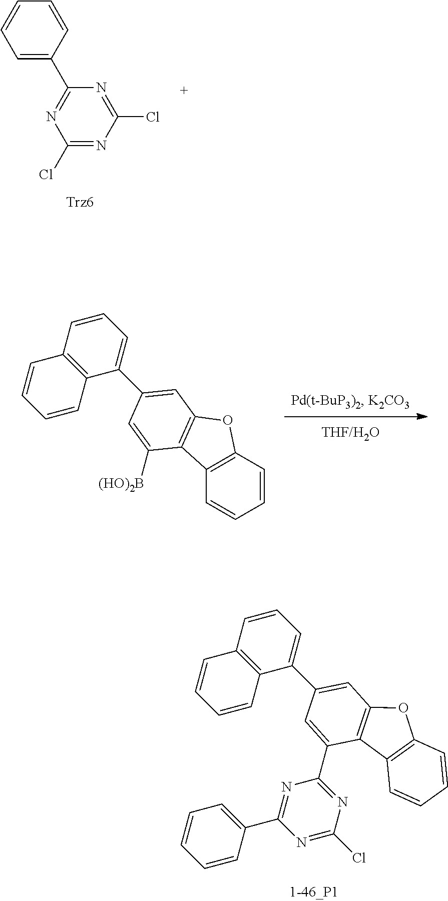 [SMILES: Clc1nc(Cl)nc(-c2ccccc2)n1 ; OB(O)c1cc(-c2cccc3ccccc23)cc2oc3ccccc3c12 ; Clc1nc(-c2ccccc2)nc(-c2cc(-c3cccc4ccccc34)cc3oc4ccccc4c23)n1]

### Step 1) Synthesis of Compound 1-46_P1

Compound Trz6 (15 g, 66.4 mmol) and (3-(naphthalen-1-yl)dibenzo[b,d]furan-1-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-46_P1 of 19.9 g. (Yield 62%, MS: [M+H]⁺=484).

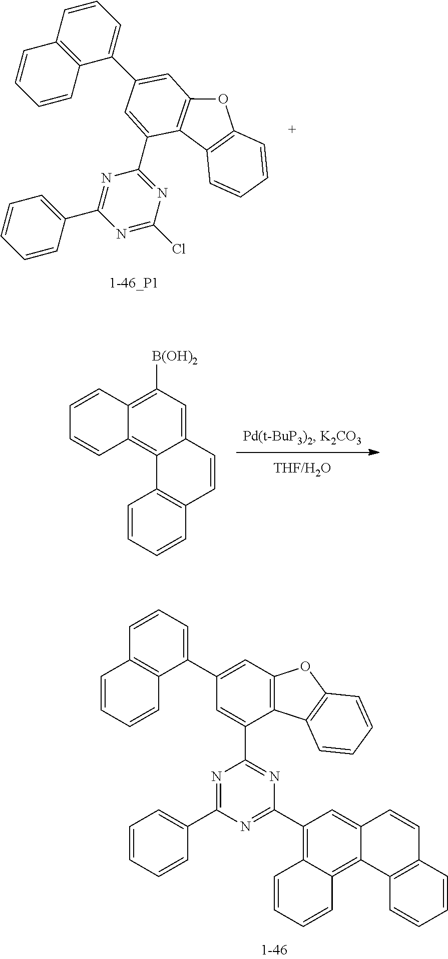 [SMILES: Clc1nc(-c2ccccc2)nc(-c2cc(-c3cccc4ccccc34)cc3oc4ccccc4c23)n1 ; OB(O)c1cc2ccc3ccccc3c2c2ccccc12 ; c1ccc(-c2nc(-c3cc4ccc5ccccc5c4c4ccccc34)nc(-c3cc(-c4cccc5ccccc45)cc4oc5ccccc5c34)n2)cc1]

### Step 2) Synthesis of Compound 1-46

Compound 1-46_P1 (15 g, 31 mmol) prepared in the above Step 1) and benzo[c]phenanthren-5-yl boronic acid (8.9 g, 32.5 mmol) were added to 300 mL of tetrahydrofuran (THF), and the mixture was stirred and refluxed. Afterwards, potassium carbonate (12.9 g, 93 mmol) was dissolved in 39 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-46 of 13.8 g. (Yield 63%, MS: [M+H]⁺=676).

### Synthesis Example 1-47

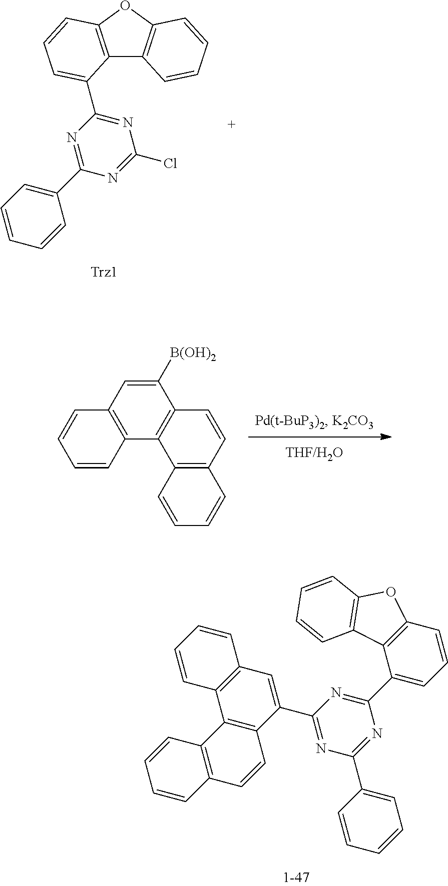 [SMILES: Clc1nc(-c2ccccc2)nc(-c2cccc3oc4ccccc4c23)n1 ; OB(O)c1cc2ccccc2c2c1ccc1ccccc12 ; c1ccc(-c2nc(-c3cc4ccccc4c4c3ccc3ccccc34)nc(-c3cccc4oc5ccccc5c34)n2)cc1]

Compound Trz1 (15 g, 41.9 mmol) and benzo[c]phenanthren-6-yl boronic acid (12 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-47 of 16.3 g. (Yield 71%, MS: [M+H]⁺=550).

### Synthesis Example 1-48

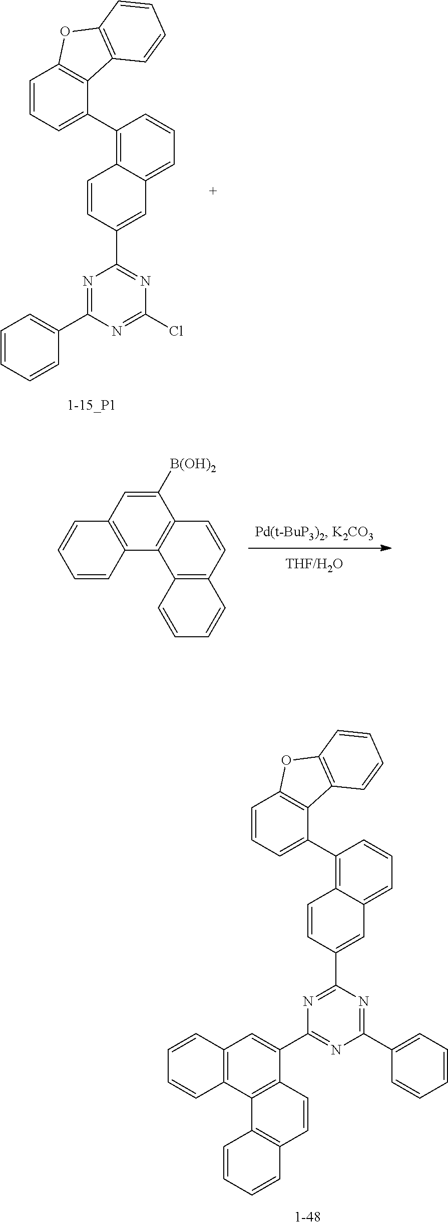 [SMILES: Clc1nc(-c2ccccc2)nc(-c2ccc3c(-c4cccc5oc6ccccc6c45)cccc3c2)n1 ; OB(O)c1cc2ccccc2c2c1ccc1ccccc12 ; c1ccc(-c2nc(-c3ccc4c(-c5cccc6oc7ccccc7c56)cccc4c3)nc(-c3cc4ccccc4c4c3ccc3ccccc34)n2)cc1]

Compound 1-15_P1 (15 g, 31 mmol) and benzo[c]phenanthren-6-yl boronic acid (8.9 g, 32.5 mmol) were added to 300 ml of tetrahydrofuran (THF), and the mixture was stirred and refluxed. Afterwards, potassium carbonate (12.9 g, 93 mmol) was dissolved in 39 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-48 of 15.3 g. (Yield 73%, MS: [M+H]⁺=676).

### Synthesis Example 1-49

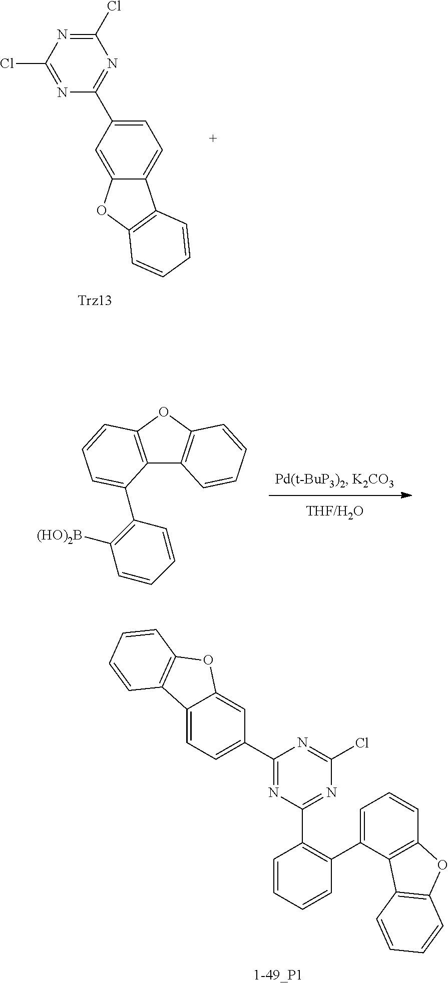

### Step 1) Synthesis of Compound 1-49_P1

Compound Trz13 (15 g, 47.4 mmol) and (2-(dibenzo[b,d]furan-1-yl)phenyl) boronic acid (14.4 g, 49.8 mmol) were added to 300 mL of tetrahydrofuran (THF), and the mixture was stirred and refluxed. Afterwards, potassium carbonate (19.7 g, 142.3 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-49_P1 of 18.4 g. (Yield 74%, MS: [M+H]⁺=524).

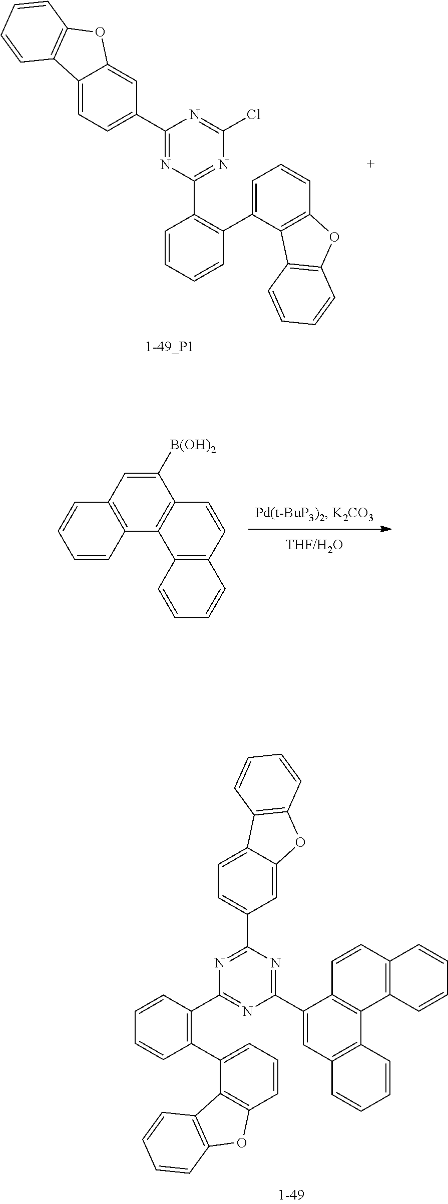 [SMILES: Clc1nc(-c2ccc3c(c2)oc2ccccc23)nc(-c2ccccc2-c2cccc3oc4ccccc4c23)n1 ; OB(O)c1cc2ccccc2c2c1ccc1ccccc12 ; c1ccc(-c2cccc3oc4ccccc4c23)c(-c2nc(-c3ccc4c(c3)oc3ccccc34)nc(-c3cc4ccccc4c4c3ccc3ccccc34)n2)c1]

### Step 2) Synthesis of Compound 1-49

Compound 1-49_P1 (15 g, 28.6 mmol) prepared in the above Step 1) and benzo[c]phenanthren-6-yl boronic acid (8.2 g, 30.1 mmol) were added to 300 ml of tetrahydrofuran (THF), and the mixture was stirred and refluxed. Afterwards, potassium carbonate (11.9 g, 85.9 mmol) was dissolved in 36 mL of water, and then added thereto. Thereafter, it was stirred sufficiently, followed by adding bis(tri-tert-butylphosphine) palladium (0) (0.1 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-49 of 13.3 g. (Yield 65%, MS: [M+H]⁺=716).

### Synthesis Example 1-50

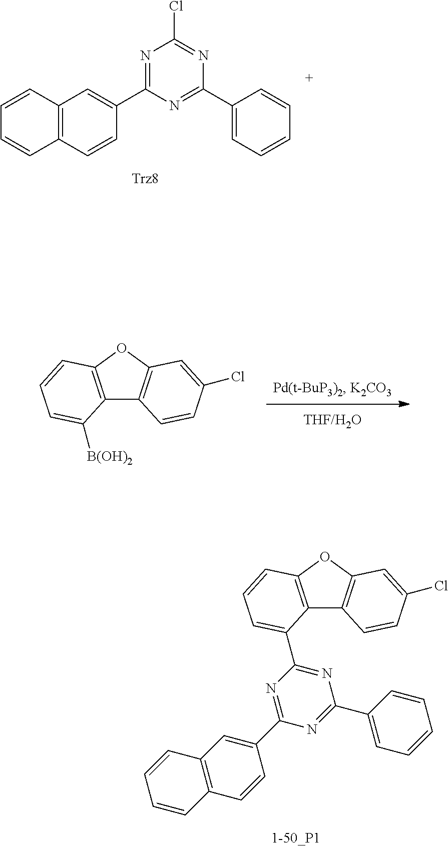 [SMILES: Clc1nc(-c2ccccc2)nc(-c2ccc3ccccc3c2)n1 ; OB(O)c1cccc2oc3cc(Cl)ccc3c12 ; Clc1ccc2c(c1)oc1cccc(-c3nc(-c4ccccc4)nc(-c4ccc5ccccc5c4)n3)c12]

### Step 1) Synthesis of Compound 1-50_P1

Compound Trz8 (15 g, 47.2 mmol) and (7-chlorodibenzo[b,d]furan-1-yl) boronic acid (12.2 g, 49.6 mmol) were added to 300 mL of tetrahydrofuran (THF), and the mixture was stirred and refluxed. Afterwards, potassium carbonate (19.6 g, 141.6 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-50_P1 of 14.4 g. (Yield 63%, MS: [M+H]⁺=484).

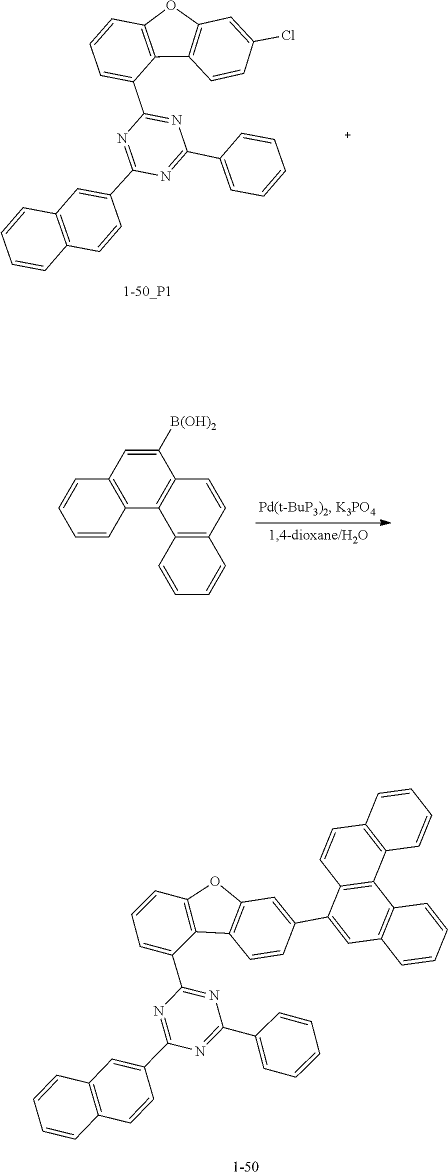 [SMILES: Clc1ccc2c(c1)oc1cccc(-c3nc(-c4ccccc4)nc(-c4ccc5ccccc5c4)n3)c12 ; OB(O)c1cc2ccccc2c2c1ccc1ccccc12 ; c1ccc(-c2nc(-c3ccc4ccccc4c3)nc(-c3cccc4oc5cc(-c6cc7ccccc7c7c6ccc6ccccc67)ccc5c34)n2)cc1]

### Step 2) Synthesis of Compound 1-50

Compound 1-50_P1 (15 g, 31 mmol) prepared in the above Step 1) and benzo[c]phenanthren-6-yl boronic acid (8.9 g, 32.5 mmol) were added to 300 mL of 1,4-dioxane, and the mixture was stirred and refluxed. Afterwards, potassium phosphate (19.7 g, 93 mmol) was dissolved in 59 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-50 of 12.8 g. (Yield 61%, MS: [M+H]⁺=676).

### Synthesis Example 1-51

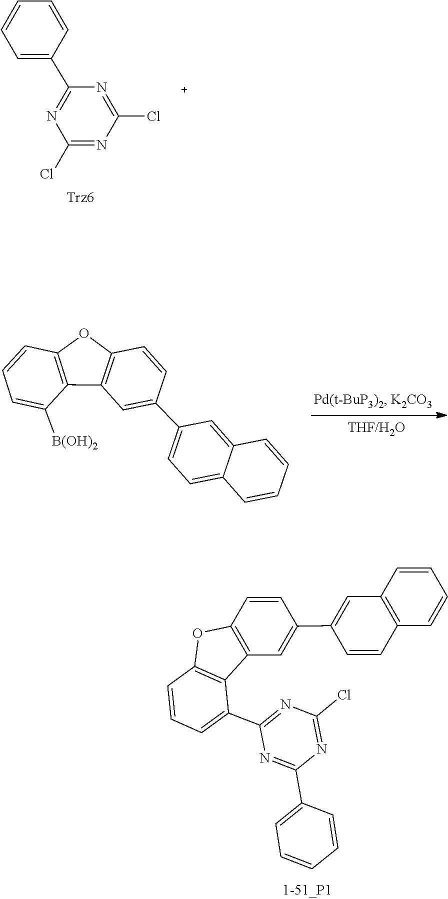 [SMILES: Clc1nc(Cl)nc(-c2ccccc2)n1 ; OB(O)c1cccc2oc3ccc(-c4ccc5ccccc5c4)cc3c12 ; Clc1nc(-c2ccccc2)nc(-c2cccc3oc4ccc(-c5ccc6ccccc6c5)cc4c23)n1]

### Step 1) Synthesis of Compound 1-51_P1

Compound Trz6 (15 g, 66.4 mmol) and (8-(naphthalen-2-yl)dibenzo[b,d]furan-1-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 59 mL of water, and then added thereto. Thereafter, it was stirred sufficiently, followed by adding bis(tri-tert-butylphosphine) palladium (0) (0.3 g, 0.7 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-51_P1 of 19.2 g. (Yield 60%, MS: [M+H]⁺=484).

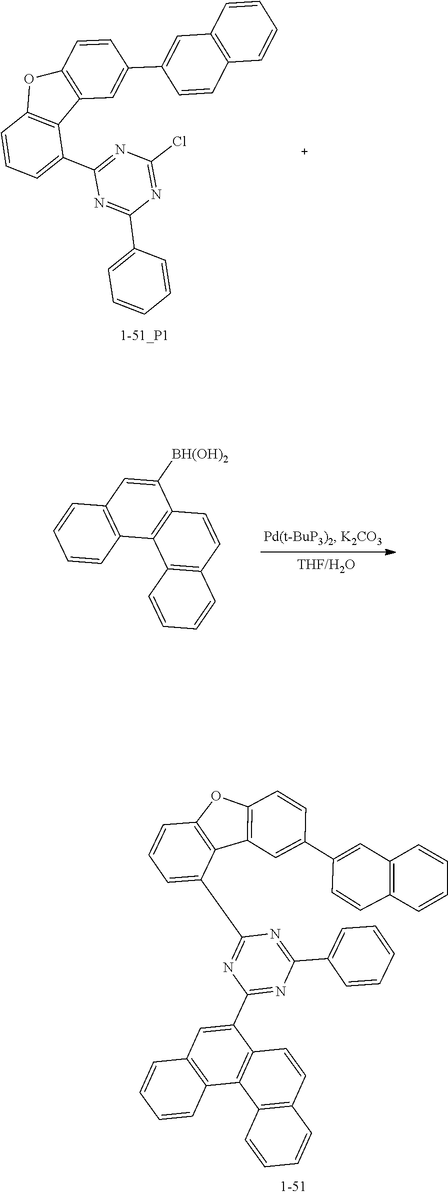

### Step 2) Synthesis of Compound 1-51

Compound 1-51_P1 (15 g, 31 mmol) prepared in the above Step 1) and benzo[c]phenanthren-6-yl boronic acid (8.9 g, 32.5 mmol) were added to 300 ml of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-51 of 14.2 g. (Yield 68%, MS: [M+H]⁺=676).

### Synthesis Example 1-52

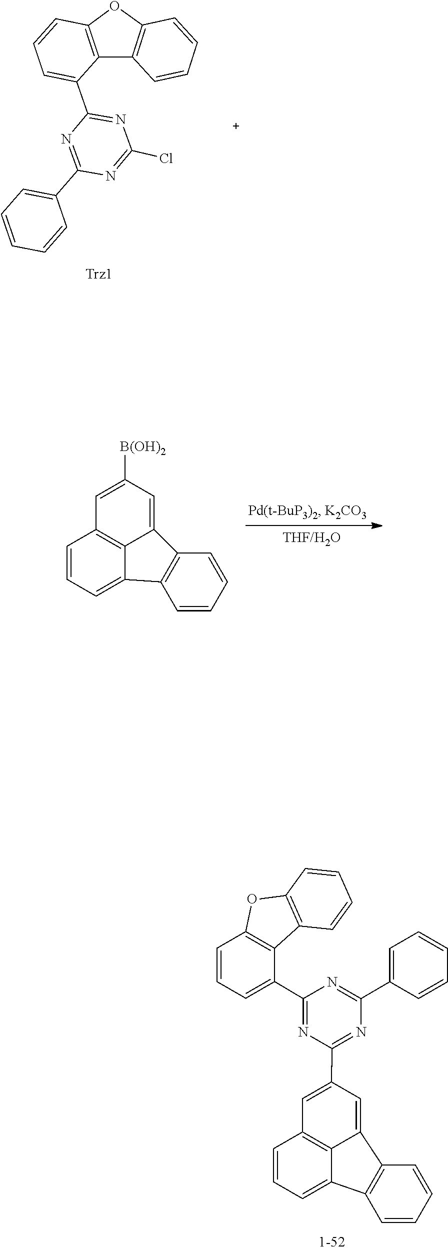 [SMILES: Clc1nc(-c2ccccc2)nc(-c2cccc3oc4ccccc4c23)n1 ; OB(O)c1cc2c3c(cccc3c1)-c1ccccc1-2 ; c1ccc(-c2nc(-c3cc4c5c(cccc5c3)-c3ccccc3-4)nc(-c3cccc4oc5ccccc5c34)n2)cc1]

Compound Trz1 (15 g, 41.9 mmol) and fluoranthen-2-yl boronic acid (10.8 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-52 of 14.9 g. (Yield 68%, MS: [M+H]⁺=524).

### Synthesis Example 1-53

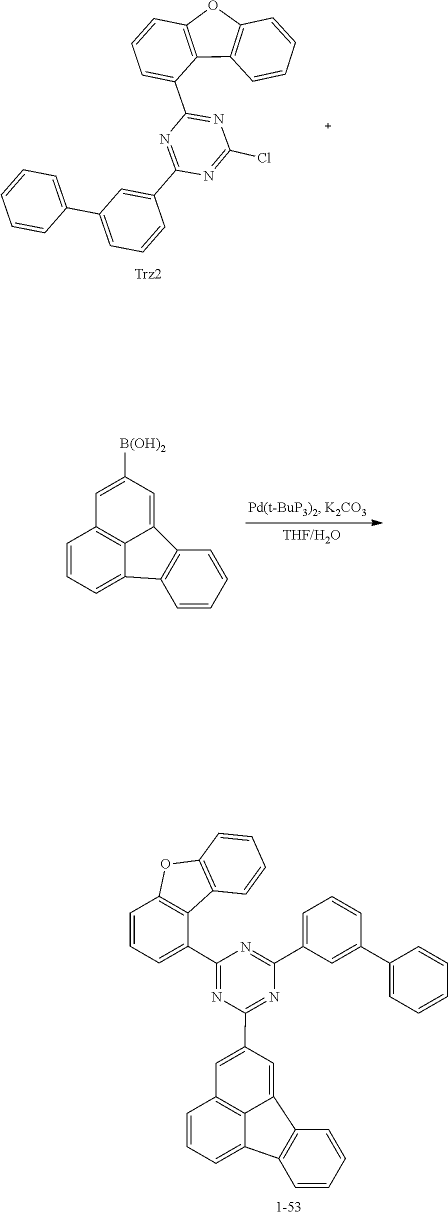 [SMILES: Clc1nc(-c2cccc(-c3ccccc3)c2)nc(-c2cccc3oc4ccccc4c23)n1 ; OB(O)c1cc2c3c(cccc3c1)-c1ccccc1-2 ; c1ccc(-c2cccc(-c3nc(-c4cc5c6c(cccc6c4)-c4ccccc4-5)nc(-c4cccc5oc6ccccc6c45)n3)c2)cc1]

Compound Trz2 (15 g, 34.6 mmol) and fluoranthen-2-yl boronic acid (8.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-53 of 14.7 g. (Yield 71%, MS: [M+H]⁺=600).

### Synthesis Example 1-54

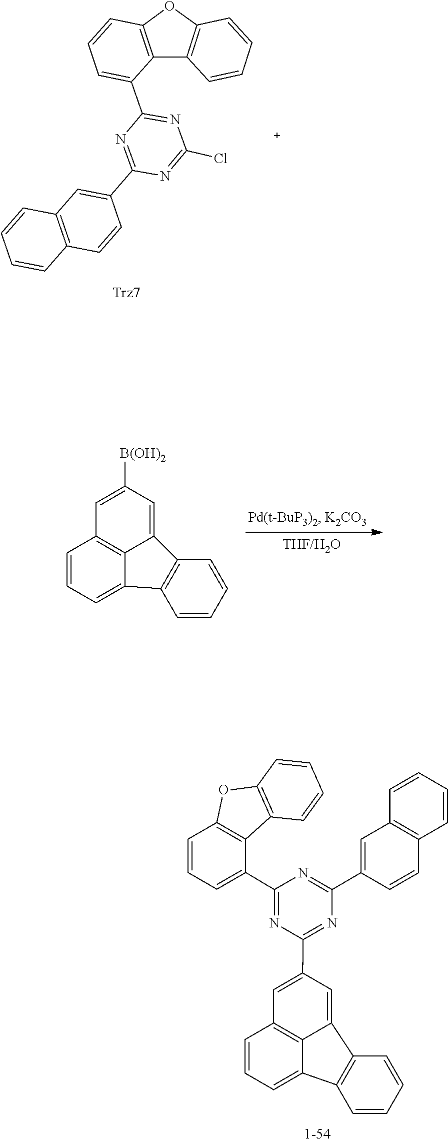 [SMILES: Clc1nc(-c2ccc3ccccc3c2)nc(-c2cccc3oc4ccccc4c23)n1 ; OB(O)c1cc2c3c(cccc3c1)-c1ccccc1-2 ; c1ccc2c(c1)-c1cccc3cc(-c4nc(-c5ccc6ccccc6c5)nc(-c5cccc6oc7ccccc7c56)n4)cc-2c13]

Compound Trz7 (15 g, 36.8 mmol) and fluoranthen-2-yl boronic acid (9.5 g, 38.6 mmol) were added to 300 mL of tetrahydrofuran (THF), and the mixture was stirred and refluxed. Afterwards, potassium carbonate (15.2 g, 110.3 mmol) was dissolved in 43 mL of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-54 of 13.7 g. (Yield 65%, MS: [M+H]⁺=574).

### Synthesis Example 1-55

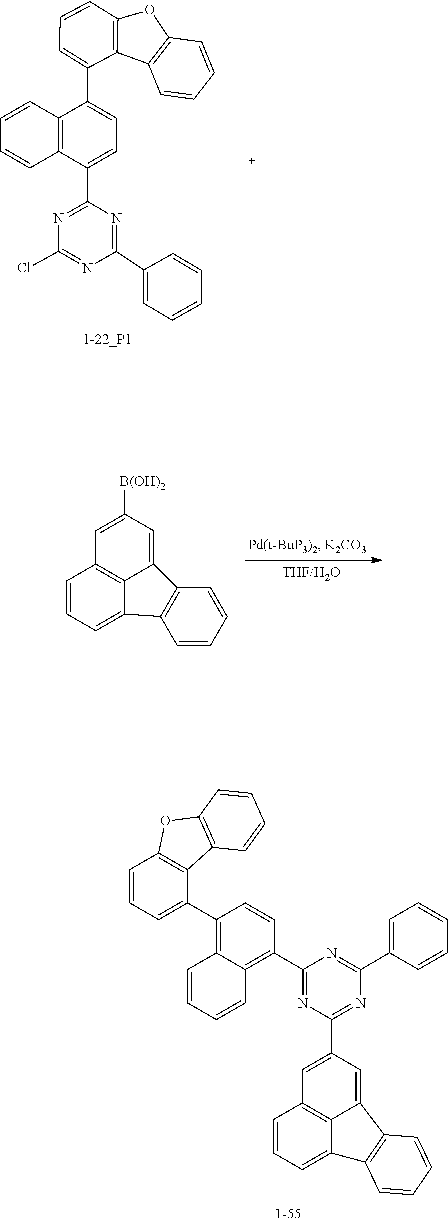 [SMILES: Clc1nc(-c2ccccc2)nc(-c2ccc(-c3cccc4oc5ccccc5c34)c3ccccc23)n1 ; OB(O)c1cc2c3c(cccc3c1)-c1ccccc1-2 ; c1ccc(-c2nc(-c3cc4c5c(cccc5c3)-c3ccccc3-4)nc(-c3ccc(-c4cccc5oc6ccccc6c45)c4ccccc34)n2)cc1]

Compound 1-22_P1 (15 g, 31 mmol) and fluoranthen-2-yl boronic acid (8 g, 32.5 mmol) were added to 300 mL of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-55 of 14.3 g. (Yield 71%, MS: [M+H]⁺=650).

### Synthesis Example 1-56

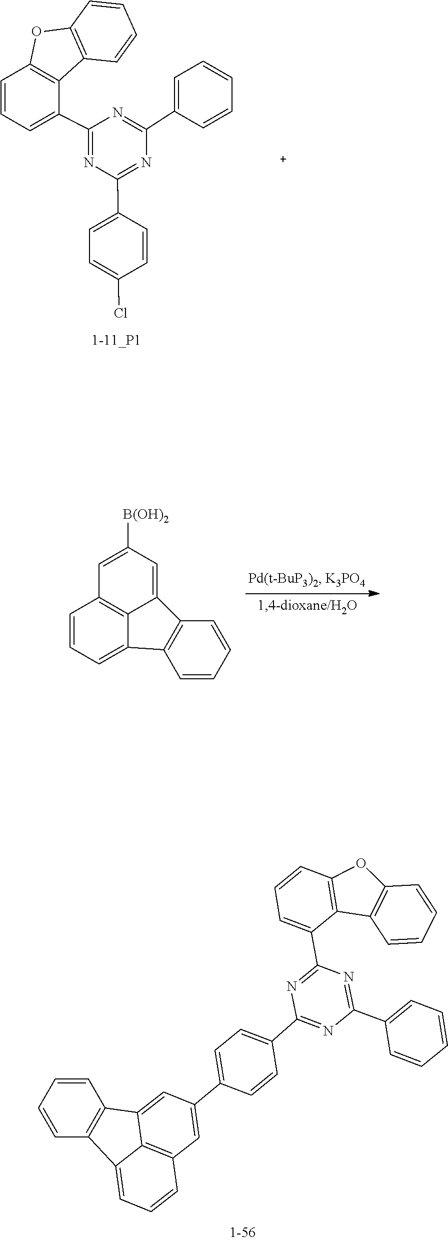 [SMILES: Clc1ccc(-c2nc(-c3ccccc3)nc(-c3cccc4oc5ccccc5c34)n2)cc1 ; OB(O)c1cc2c3c(cccc3c1)-c1ccccc1-2 ; c1ccc(-c2nc(-c3ccc(-c4cc5c6c(cccc6c4)-c4ccccc4-5)cc3)nc(-c3cccc4oc5ccccc5c34)n2)cc1]

Compound 1-11_P1 (15 g, 33.5 mmol) and fluoranthen-2-yl boronic acid (8.7 g, 35.2 mmol) were added to 300 mL of tetrahydrofuran (THF), and the mixture was stirred and refluxed. Afterwards, potassium carbonate (13.9 g, 100.5 mmol) was dissolved in 42 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-56 of 12.8 g. (Yield 64%, MS: [M+H]⁺=600).

### Synthesis Example 1-57

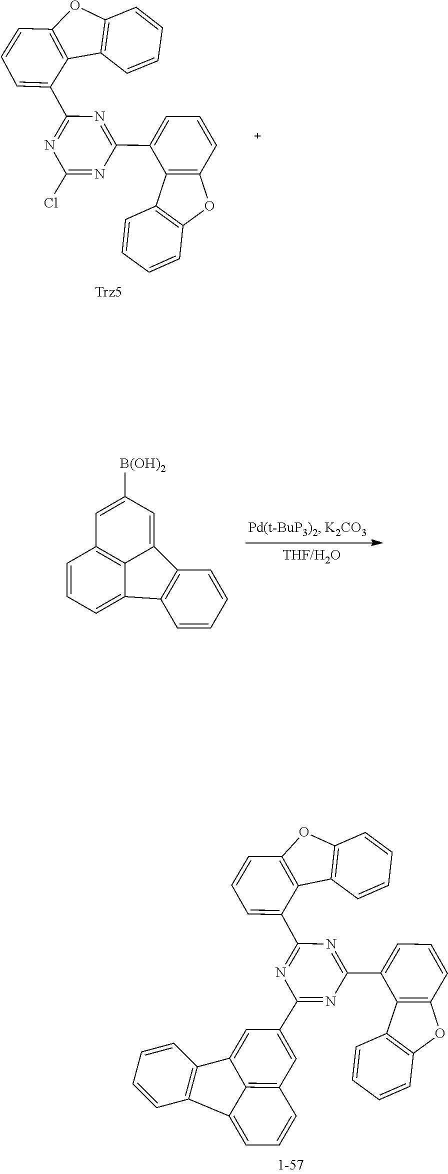 [SMILES: Clc1nc(-c2cccc3oc4ccccc4c23)nc(-c2cccc3oc4ccccc4c23)n1 ; OB(O)c1cc2c3c(cccc3c1)-c1ccccc1-2 ; c1ccc2c(c1)-c1cccc3cc(-c4nc(-c5cccc6oc7ccccc7c56)nc(-c5cccc6oc7ccccc7c56)n4)cc-2c13]

Compound Trz5 (15 g, 33.5 mmol) and fluoranthen-2-yl boronic acid (8.7 g, 35.2 mmol) were added to 300 mL of tetrahydrofuran (THF), and the mixture was stirred and refluxed. Afterwards, potassium carbonate (13.9 g, 100.5 mmol) was dissolved in 42 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-57 of 15 g. (Yield 73%, MS: [M+H]⁺=614).

### Synthesis Example 1-58

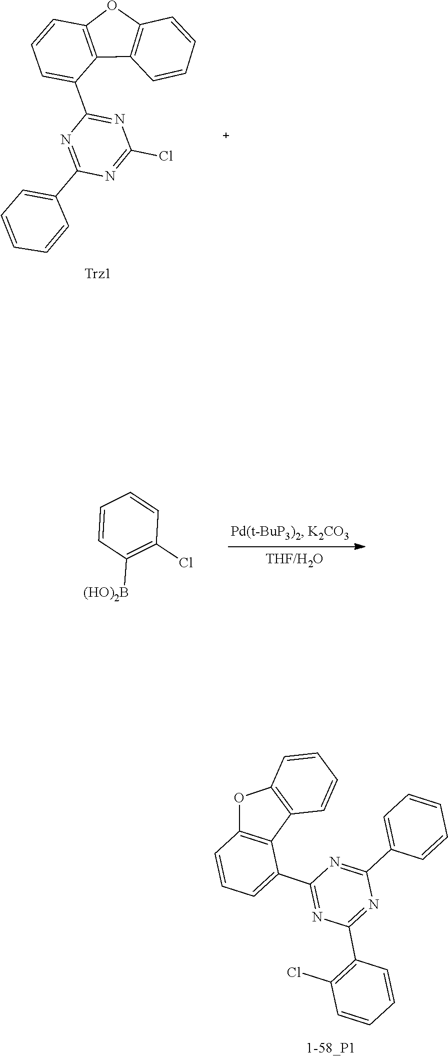 [SMILES: Clc1nc(-c2ccccc2)nc(-c2cccc3oc4ccccc4c23)n1 ; OB(O)c1ccccc1Cl ; Clc1ccccc1-c1nc(-c2ccccc2)nc(-c2cccc3oc4ccccc4c23)n1]

### Step 1) Synthesis of Compound 1-58_P1

Compound Trz1 (15 g, 41.9 mmol) and (2-chlorophenyl) boronic acid (6.9 g, 44 mmol) were added to 300 mL of tetrahydrofuran (THF), and the mixture was stirred and refluxed. Afterwards, potassium carbonate (17.4 g, 125.8 mmol) was dissolved in 52 mL of water, and then added thereto. Thereafter, it was stirred sufficiently, followed by adding bis(tri-tert-butylphosphine) palladium (0) (0.2 g, 0.4 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-58_P1 of 13.1 g. (Yield 72%, MS: [M+H]⁺=434).

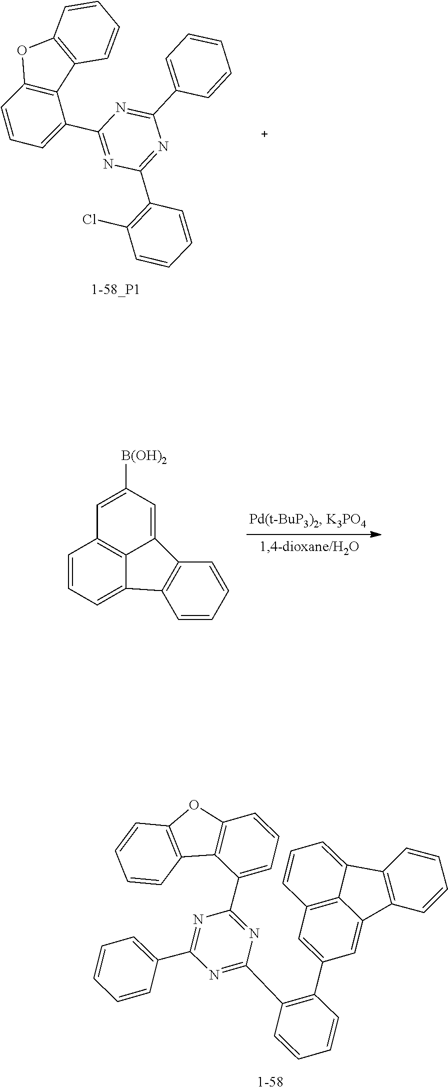 [SMILES: Clc1ccccc1-c1nc(-c2ccccc2)nc(-c2cccc3oc4ccccc4c23)n1 ; OB(O)c1cc2c3c(cccc3c1)-c1ccccc1-2 ; c1ccc(-c2nc(-c3ccccc3-c3cc4c5c(cccc5c3)-c3ccccc3-4)nc(-c3cccc4oc5ccccc5c34)n2)cc1]

### Step 2) Synthesis of Compound 1-58

Compound 1-58_P1 (15 g, 34.6 mmol) prepared in the above Step 1) and fluoranthen-2-yl boronic acid (8.9 g, 36.3 mmol) were added to 300 mL of 1,4-dioxane, and the mixture was stirred and refluxed. Afterwards, potassium phosphate (22 g, 103.7 mmol) was dissolved in 66 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-58 of 12.8 g. (Yield 62%, MS: [M+H]⁺=600).

### Synthesis Example 1-59

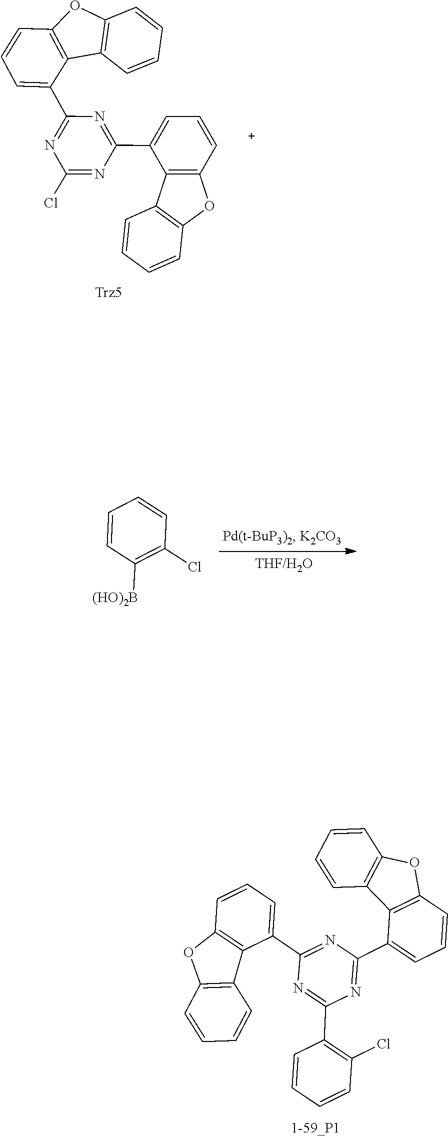 [SMILES: Clc1nc(-c2cccc3oc4ccccc4c23)nc(-c2cccc3oc4ccccc4c23)n1 ; OB(O)c1ccccc1Cl ; Clc1ccccc1-c1nc(-c2cccc3oc4ccccc4c23)nc(-c2cccc3oc4ccccc4c23)n1]

### Step 1) Synthesis of Compound 1-59_P1

Compound Trz5 (15 g, 33.5 mmol) and (2-chlorophenyl) boronic acid (5.5 g, 35.2 mmol) were added to 300 mL of tetrahydrofuran (THF), and the mixture was stirred and refluxed. Afterwards, potassium carbonate (13.9 g, 100.5 mmol) was dissolved in 42 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-59_P1 of 10.9 g. (Yield 62%, MS: [M+H]⁺=524).

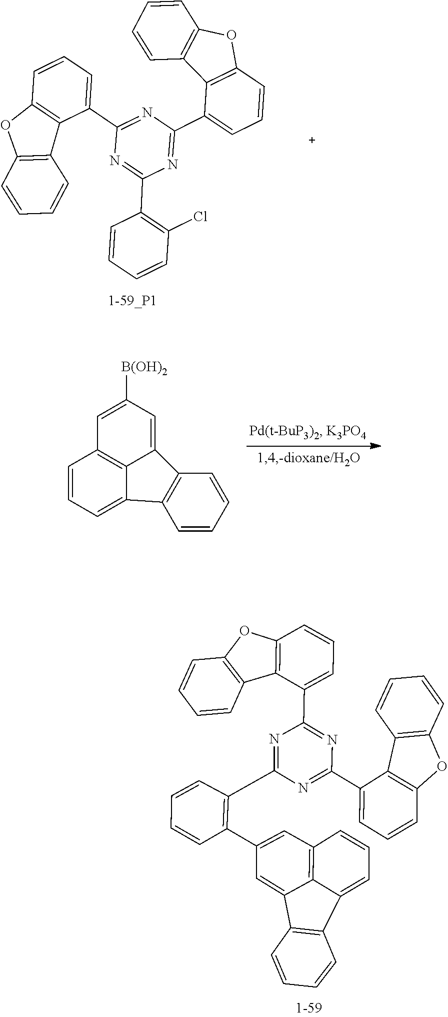 [SMILES: Clc1ccccc1-c1nc(-c2cccc3oc4ccccc4c23)nc(-c2cccc3oc4ccccc4c23)n1 ; OB(O)c1cc2c3c(cccc3c1)-c1ccccc1-2 ; c1ccc(-c2nc(-c3cccc4oc5ccccc5c34)nc(-c3cccc4oc5ccccc5c34)n2)c(-c2cc3c4c(cccc4c2)-c2ccccc2-3)c1]

### Step 2) Synthesis of Compound 1-59

Compound 1-59_P1 (15 g, 28.6 mmol) prepared in the above Step 1) and fluoranthen-2-yl boronic acid (7.4 g, 30.1 mmol) were added to 300 mL of 1,4-dioxane, and the mixture was stirred and refluxed. Afterwards, potassium phosphate (18.2 g, 85.9 mmol) was dissolved in 55 mL of water, and then added thereto. Thereafter, it was stirred sufficiently, followed by adding bis(tri-tert-butylphosphine) palladium (0) (0.1 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-59 of 12.2 g. (Yield 62%, MS: [M+H]⁺=690).

### Synthesis Example 1-60

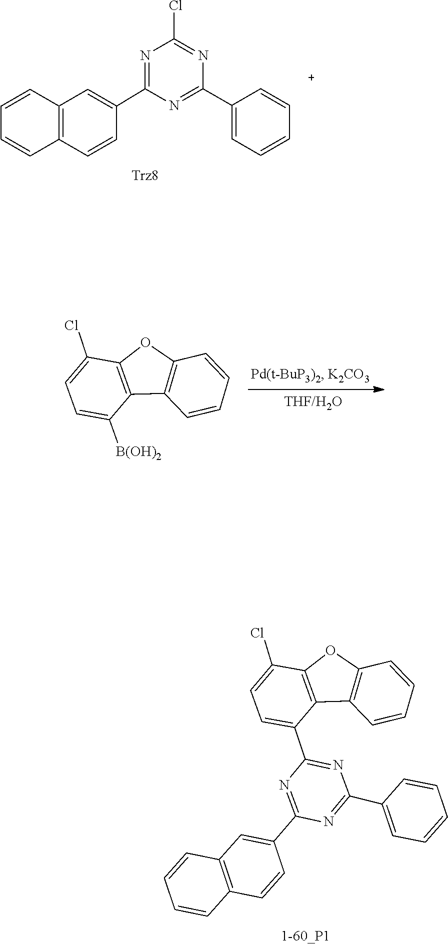 [SMILES: Clc1nc(-c2ccccc2)nc(-c2ccc3ccccc3c2)n1 ; OB(O)c1ccc(Cl)c2oc3ccccc3c12 ; Clc1ccc(-c2nc(-c3ccccc3)nc(-c3ccc4ccccc4c3)n2)c2c1oc1ccccc12]

### Step 1) Synthesis of Compound 1-60_P1

Compound Trz8 (15 g, 47.2 mmol) and (4-chlorodibenzo[b,d]furan-1-yl) boronic acid (12.2 g, 49.6 mmol) were added to 300 mL of tetrahydrofuran (THF), and the mixture was stirred and refluxed. Afterwards, potassium carbonate (19.6 g, 141.6 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-60_P1 of 16.4 g. (Yield 72%, MS: [M+H]⁺=484).

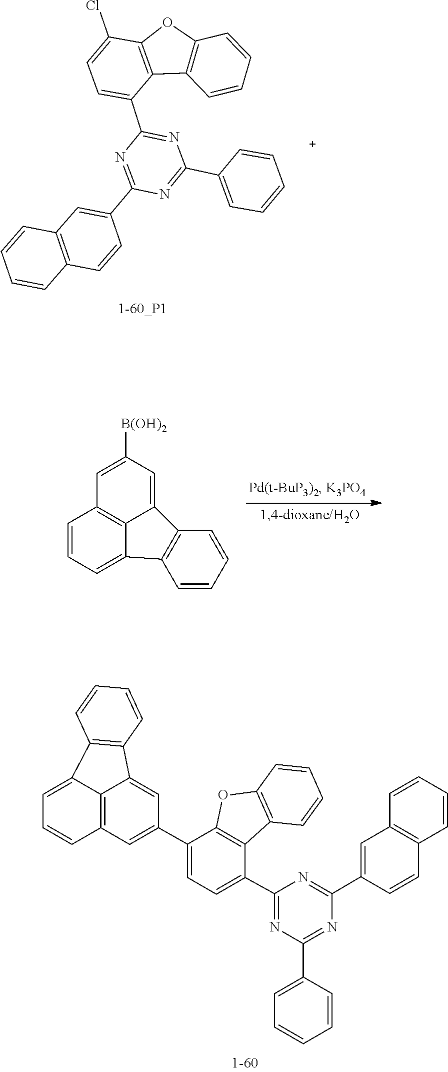 [SMILES: Clc1ccc(-c2nc(-c3ccccc3)nc(-c3ccc4ccccc4c3)n2)c2c1oc1ccccc12 ; OB(O)c1cc2c3c(cccc3c1)-c1ccccc1-2 ; c1ccc(-c2nc(-c3ccc4ccccc4c3)nc(-c3ccc(-c4cc5c6c(cccc6c4)-c4ccccc4-5)c4oc5ccccc5c34)n2)cc1]

### Step 2) Synthesis of Compound 1-60

Compound 1-60_P1 (15 g, 31 mmol) prepared in the above Step 1) and fluoranthen-2-yl boronic acid (8 g, 32.5 mmol) were added to 300 mL of 1,4-dioxane, and the mixture was stirred and refluxed. Afterwards, potassium phosphate (19.7 g, 93 mmol) was dissolved in 59 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-60 of 13.5 g. (Yield 67%, MS: [M+H]⁺=650).

### Synthesis Example 1-61

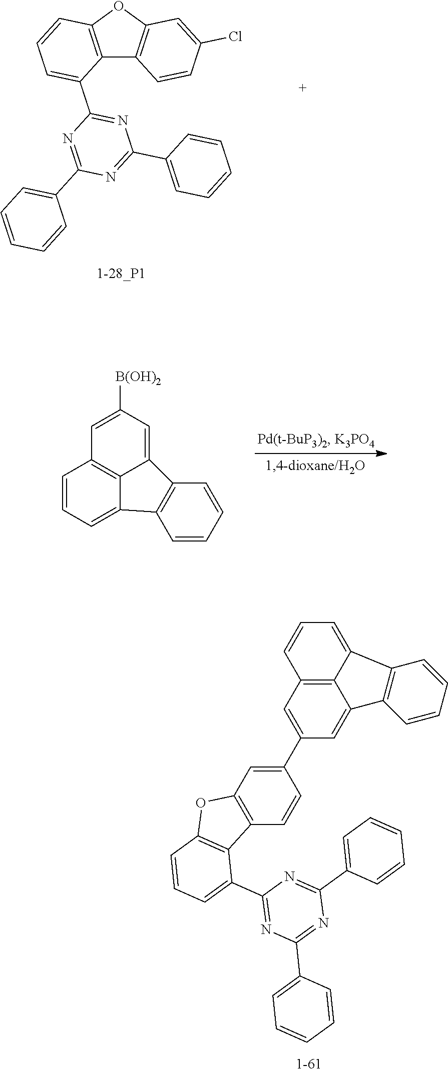 [SMILES: Clc1ccc2c(c1)oc1cccc(-c3nc(-c4ccccc4)nc(-c4ccccc4)n3)c12 ; OB(O)c1cc2c3c(cccc3c1)-c1ccccc1-2 ; c1ccc(-c2nc(-c3ccccc3)nc(-c3cccc4oc5cc(-c6cc7c8c(cccc8c6)-c6ccccc6-7)ccc5c34)n2)cc1]

Compound 1-28_P1 (15 g, 34.6 mmol) and fluoranthen-2-yl boronic acid (8.9 g, 36.3 mmol) were added to 300 mL of 1,4-dioxane, and the mixture was stirred and refluxed. Afterwards, potassium phosphate (22 g, 103.7 mmol) was dissolved in 66 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-61 of 15.3 g. (Yield 74%, MS: [M+H]⁺=600).

### Synthesis Example 1-62

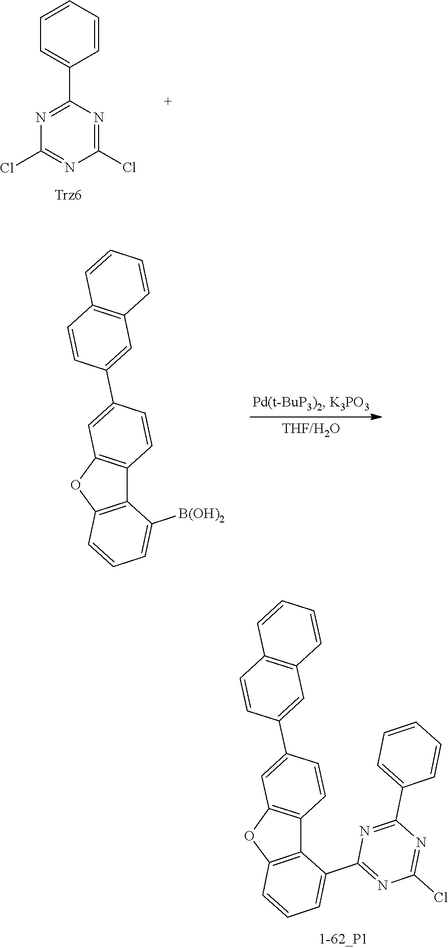 [SMILES: Clc1nc(Cl)nc(-c2ccccc2)n1 ; OB(O)c1cccc2oc3cc(-c4ccc5ccccc5c4)ccc3c12 ; Clc1nc(-c2ccccc2)nc(-c2cccc3oc4cc(-c5ccc6ccccc6c5)ccc4c23)n1]

### Step 1) Synthesis of Compound 1-62_P1

Compound Trz6 (15 g, 66.4 mmol) and (7-(naphthalen-2-yl)dibenzo[b,d]furan-1-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-62_P1 of 21.8 g. (Yield 68%, MS: [M+H]⁺=484).

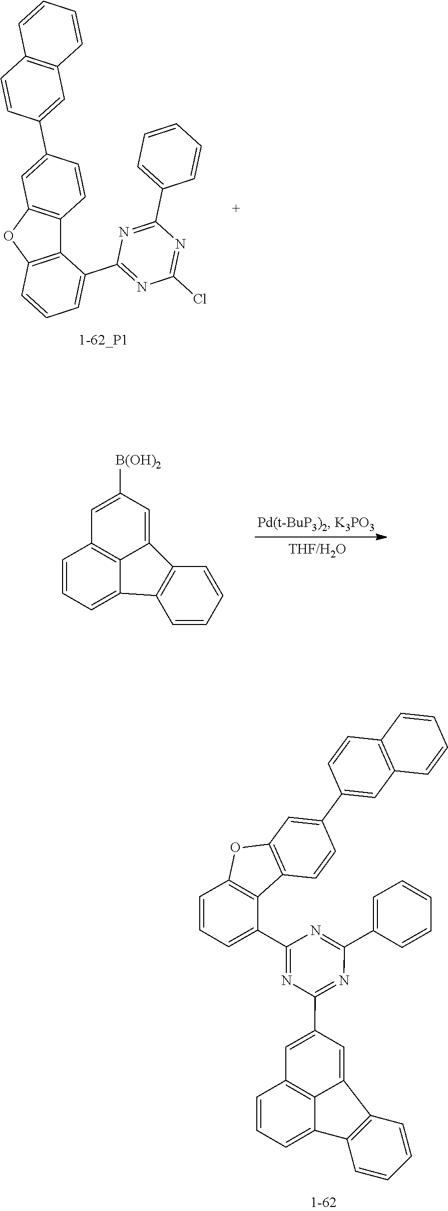 [SMILES: Clc1nc(-c2ccccc2)nc(-c2cccc3oc4cc(-c5ccc6ccccc6c5)ccc4c23)n1 ; OB(O)c1cc2c3c(cccc3c1)-c1ccccc1-2 ; c1ccc(-c2nc(-c3cc4c5c(cccc5c3)-c3ccccc3-4)nc(-c3cccc4oc5cc(-c6ccc7ccccc7c6)ccc5c34)n2)cc1]

### Step 2) Synthesis of Compound 1-62

Compound 1-62_P1 (15 g, 31 mmol) prepared in the above Step 1) and fluoranthen-2-yl boronic acid (8 g, 32.5 mmol) were added to 300 ml of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-62 of 14.5 g. (Yield 72%, MS: [M+H]⁺=650).

### Synthesis Example 1-63

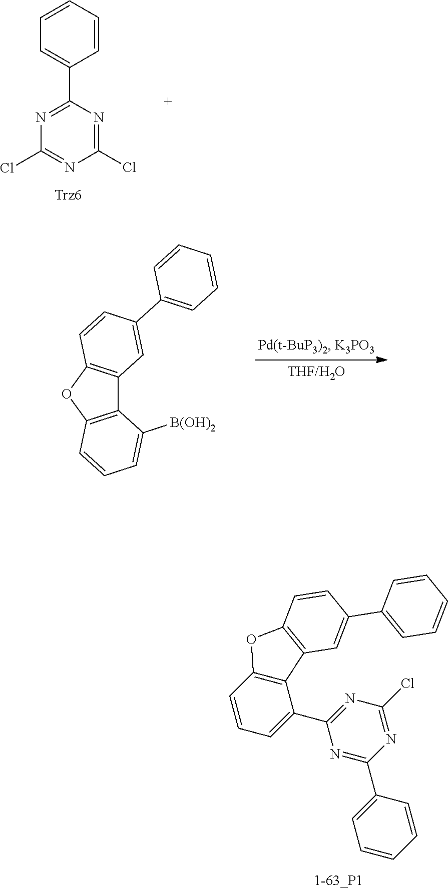 [SMILES: Clc1nc(Cl)nc(-c2ccccc2)n1 ; OB(O)c1cccc2oc3ccc(-c4ccccc4)cc3c12 ; Clc1nc(-c2ccccc2)nc(-c2cccc3oc4ccc(-c5ccccc5)cc4c23)n1]

### Step 1) Synthesis of Compound 1-63_P1

Compound Trz6 (15 g, 66.4 mmol) and (8-phenyldibenzo[b,d]furan-1-yl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-63_P1 of 17.8 g. (Yield 62%, MS: [M+H]⁺=434).

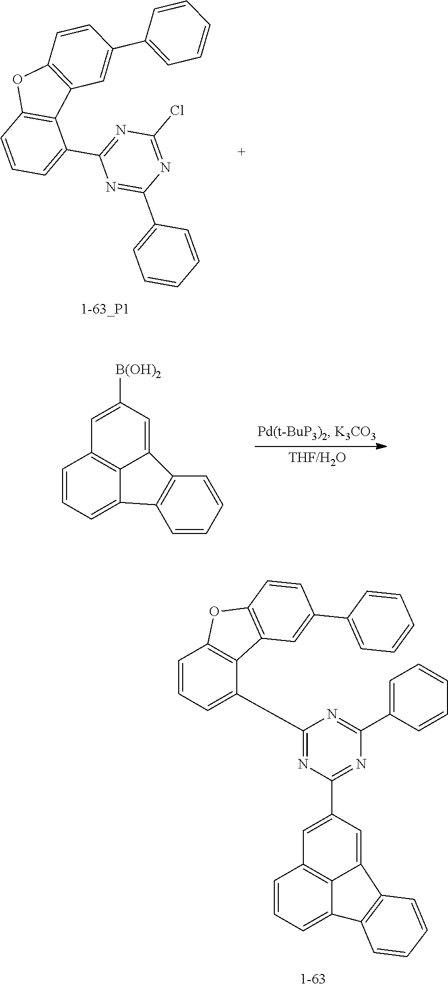 [SMILES: Clc1nc(-c2ccccc2)nc(-c2cccc3oc4ccc(-c5ccccc5)cc4c23)n1 ; OB(O)c1cc2c3c(cccc3c1)-c1ccccc1-2 ; c1ccc(-c2ccc3oc4cccc(-c5nc(-c6ccccc6)nc(-c6cc7c8c(cccc8c6)-c6ccccc6-7)n5)c4c3c2)cc1]

### Step 2) Synthesis of Compound 1-63

Compound 1-63_P1 (15 g, 34.6 mmol) prepared in the above Step 1) and fluoranthen-2-yl boronic acid (8.9 g, 36.3 mmol) were added to 300 ml of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-63 of 15.3 g. (Yield 74%, MS: [M+H]⁺=600).

### Synthesis Example 1-64

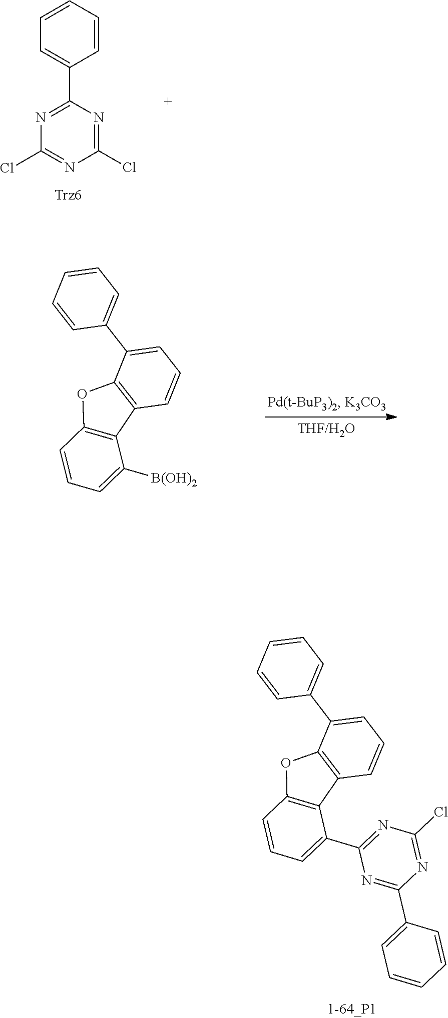 [SMILES: Clc1nc(Cl)nc(-c2ccccc2)n1 ; OB(O)c1cccc2oc3c(-c4ccccc4)cccc3c12 ; Clc1nc(-c2ccccc2)nc(-c2cccc3oc4c(-c5ccccc5)cccc4c23)n1]

### Step 1) Synthesis of Compound 1-64_P1

Compound Trz6 (15 g, 66.4 mmol) and (6-phenyldibenzo[b,d]furan-1-yl) boronic (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-64_P1 of 19.8 g. (Yield 69%, MS: [M+H]⁺=434).

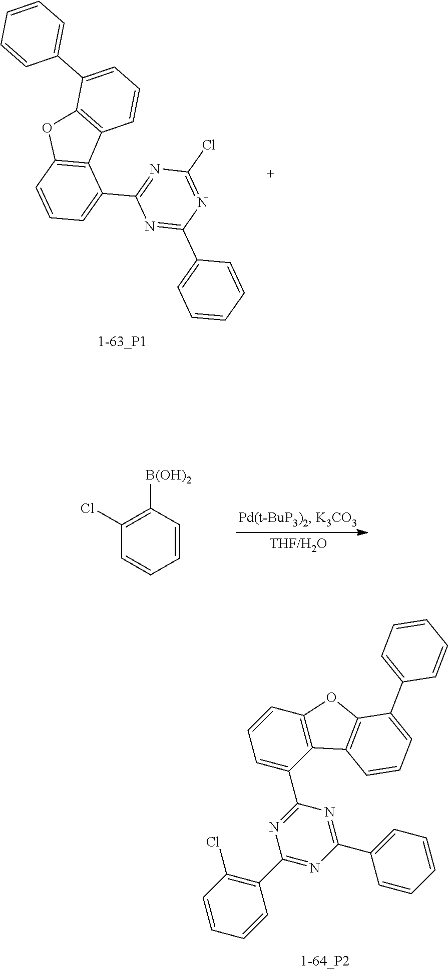 [SMILES: Clc1nc(-c2ccccc2)nc(-c2cccc3oc4c(-c5ccccc5)cccc4c23)n1 ; OB(O)c1ccccc1Cl ; Clc1ccccc1-c1nc(-c2ccccc2)nc(-c2cccc3oc4c(-c5ccccc5)cccc4c23)n1]

### Step 2) Synthesis of Compound 1-64_P2

Compound 1-64_P1 (15 g, 34.6 mmol) prepared in the above Step 1) and (2-chlorophenyl) boronic acid (5.7 g, 36.3 mmol) were added to 300 mL of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-64_P2 of 13 g. (Yield 74%, MS: [M+H]⁺=510).

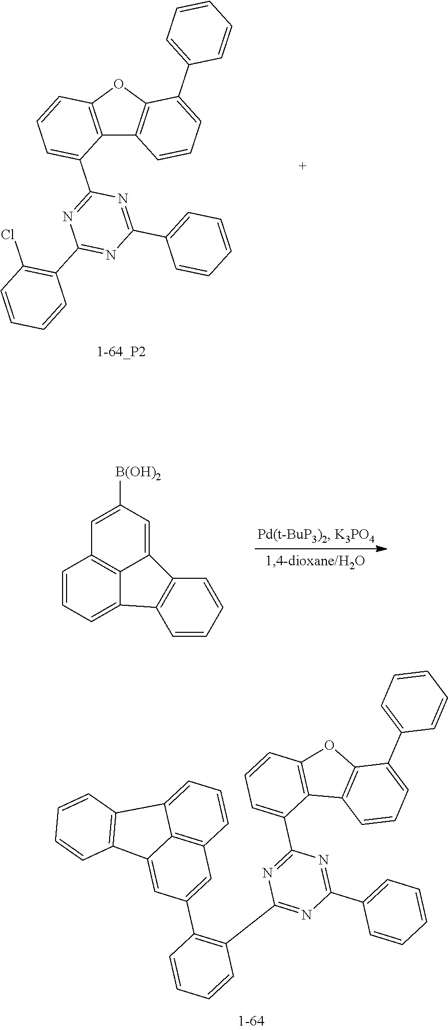 [SMILES: Clc1ccccc1-c1nc(-c2ccccc2)nc(-c2cccc3oc4c(-c5ccccc5)cccc4c23)n1 ; OB(O)c1cc2c3c(cccc3c1)-c1ccccc1-2 ; c1ccc(-c2nc(-c3ccccc3-c3cc4c5c(cccc5c3)-c3ccccc3-4)nc(-c3cccc4oc5c(-c6ccccc6)cccc5c34)n2)cc1]

### Step 3) Synthesis of Compound 1-64

Compound 1-64_P2 (15 g, 29.4 mmol) prepared in the above Step 2) and fluoranthen-2-yl boronic acid (7.6 g, 30.9 mmol) were added to 300 mL of 1,4-dioxane, and the mixture was stirred and refluxed. Afterwards, potassium phosphate (18.7 g, 88.2 mmol) was dissolved in 56 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-64 of 13.9 g. (Yield 70%, MS: [M+H]⁺=676).

### Synthesis Example 1-65

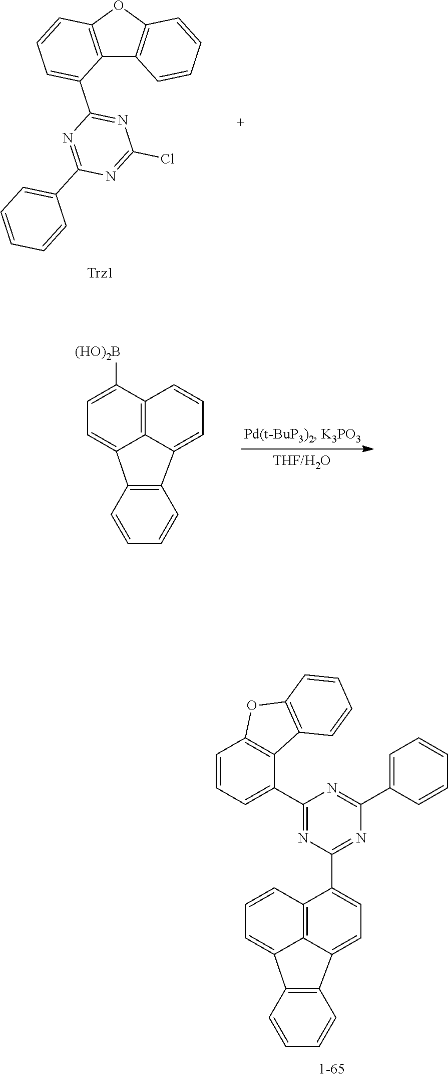 [SMILES: Clc1nc(-c2ccccc2)nc(-c2cccc3oc4ccccc4c23)n1 ; OB(O)c1ccc2c3c(cccc13)-c1ccccc1-2 ; c1ccc(-c2nc(-c3ccc4c5c(cccc35)-c3ccccc3-4)nc(-c3cccc4oc5ccccc5c34)n2)cc1]

Compound Trz1 (15 g, 41.9 mmol) and fluoranthen-3-yl boronic acid (10.8 g, 44 mmol) were added to 300 mL of tetrahydrofuran (THF), and the mixture was stirred and refluxed. Afterwards, potassium carbonate (17.4 g, 125.8 mmol) was dissolved in 52 ml of water, and then added thereto. Thereafter, it was stirred sufficiently, followed by adding bis(tri-tert-butylphosphine) palladium (0) (0.2 g, 0.4 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-65 of 13.2 g. (Yield 66%, MS: [M+H]⁺=524).

### Synthesis Example 1-66

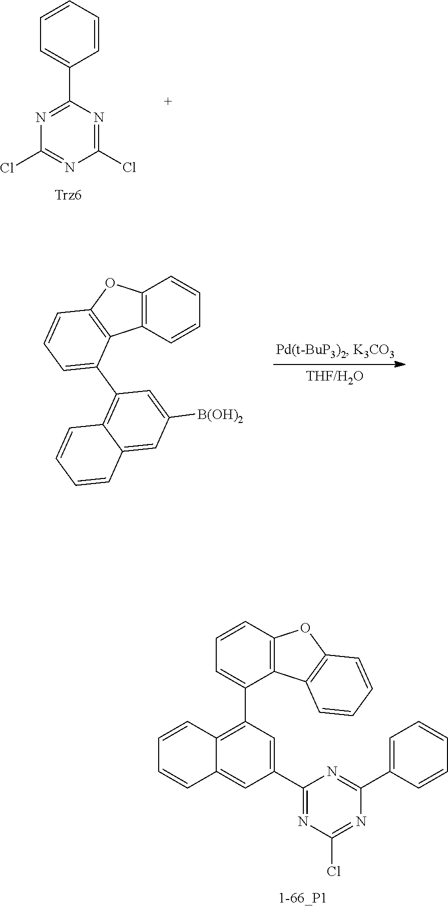 [SMILES: Clc1nc(Cl)nc(-c2ccccc2)n1 ; OB(O)c1cc(-c2cccc3oc4ccccc4c23)c2ccccc2c1 ; Clc1nc(-c2ccccc2)nc(-c2cc(-c3cccc4oc5ccccc5c34)c3ccccc3c2)n1]

### Step 1) Synthesis of Compound 1-66_P1

Compound Trz6 (15 g, 66.4 mmol) and (4-(dibenzo[b,d]furan-1-yl)naphthalen-2-yl) boronic acid (23.6 g, 69.7 mmol) were added to 300 ml of tetrahydrofuran (THF), and the mixture was stirred and refluxed. Afterwards, potassium carbonate (27.5 g, 199.1 mmol) was dissolved in 83 ml of water, and then added thereto. Thereafter, it was stirred sufficiently, followed by adding bis(tri-tert-butylphosphine) palladium (0) (0.3 g, 0.7 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-66_P1 of 20.8 g. (Yield 65%, MS: [M+H]⁺=484).

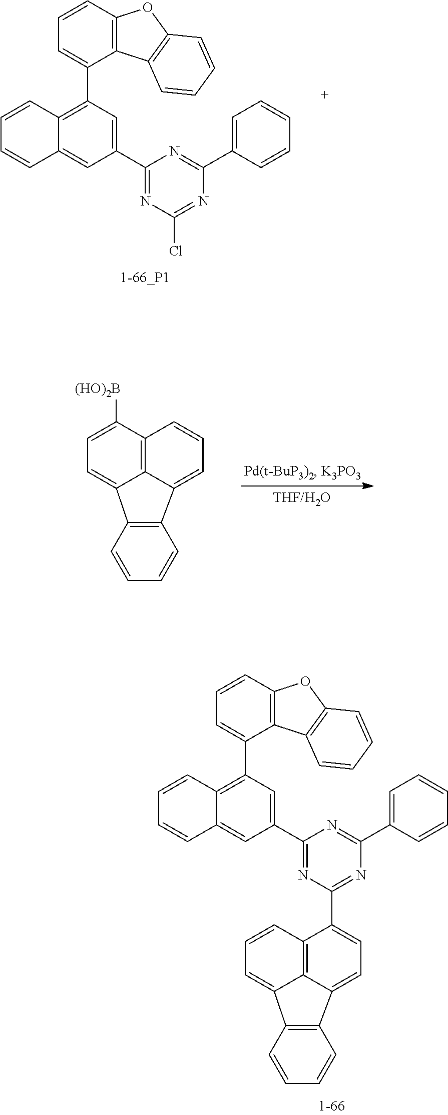 [SMILES: Clc1nc(-c2ccccc2)nc(-c2cc(-c3cccc4oc5ccccc5c34)c3ccccc3c2)n1 ; OB(O)c1ccc2c3c(cccc13)-c1ccccc1-2 ; c1ccc(-c2nc(-c3cc(-c4cccc5oc6ccccc6c45)c4ccccc4c3)nc(-c3ccc4c5c(cccc35)-c3ccccc3-4)n2)cc1]

### Step 2) Synthesis of Compound 1-66

Compound 1-66_P1 (15 g, 31 mmol) prepared in the above Step 1) and fluoranthen-3-yl boronic acid (8 g, 32.5 mmol) were added to 300 ml of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-66 of 12.9 g. (Yield 64%, MS: [M+H]⁺=650).

### Synthesis Example 1-67

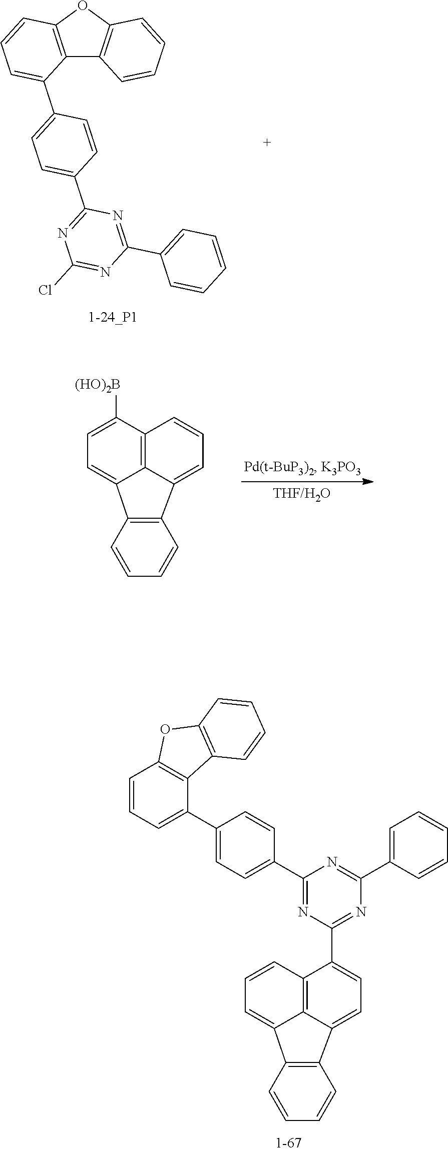 [SMILES: Clc1nc(-c2ccccc2)nc(-c2ccc(-c3cccc4oc5ccccc5c34)cc2)n1 ; OB(O)c1ccc2c3c(cccc13)-c1ccccc1-2 ; c1ccc(-c2nc(-c3ccc(-c4cccc5oc6ccccc6c45)cc3)nc(-c3ccc4c5c(cccc35)-c3ccccc3-4)n2)cc1]

Compound 1-24_P1 (15 g, 34.6 mmol) and fluoranthen-3-yl boronic acid (8.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-67 of 14.3 g. (Yield 69%, MS: [M+H]⁺=600).

### Synthesis Example 1-68

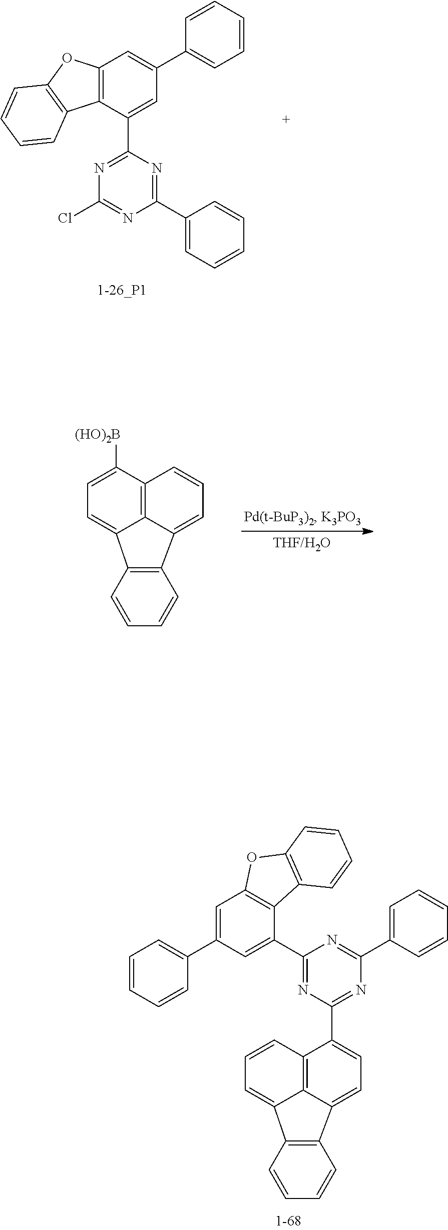 [SMILES: Clc1nc(-c2ccccc2)nc(-c2cc(-c3ccccc3)cc3oc4ccccc4c23)n1 ; OB(O)c1ccc2c3c(cccc13)-c1ccccc1-2 ; c1ccc(-c2cc(-c3nc(-c4ccccc4)nc(-c4ccc5c6c(cccc46)-c4ccccc4-5)n3)c3c(c2)oc2ccccc23)cc1]

Compound 1-26_P1 (15 g, 34.6 mmol) and fluoranthen-3-yl boronic acid (8.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-68 of 15.3 g. (Yield 74%, MS: [M+H]⁺=600).

### Synthesis Example 1-69

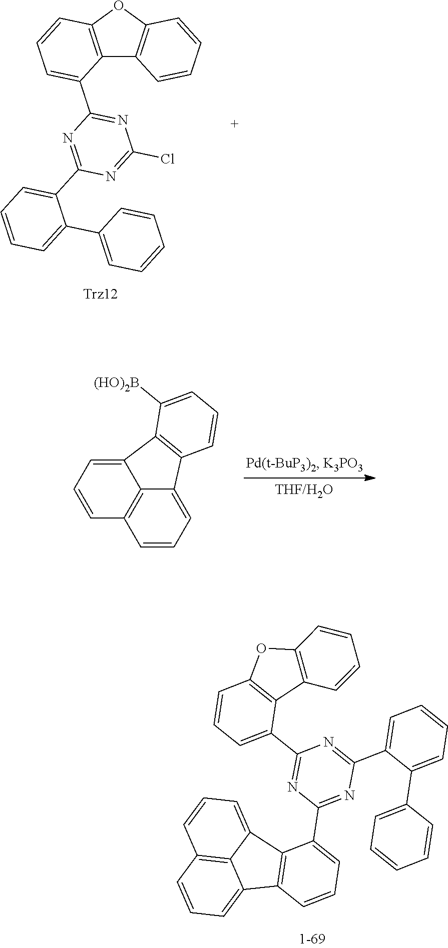 [SMILES: Clc1nc(-c2ccccc2-c2ccccc2)nc(-c2cccc3oc4ccccc4c23)n1 ; OB(O)c1cccc2c1-c1cccc3cccc-2c13 ; c1ccc(-c2ccccc2-c2nc(-c3cccc4c3-c3cccc5cccc-4c35)nc(-c3cccc4oc5ccccc5c34)n2)cc1]

Compound Trz12 (15 g, 34.6 mmol) and fluoranthen-7-yl boronic acid (8.9 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-69 of 14.1 g. (Yield 68%, MS: [M+H]⁺=600).

### Synthesis Example 1-70

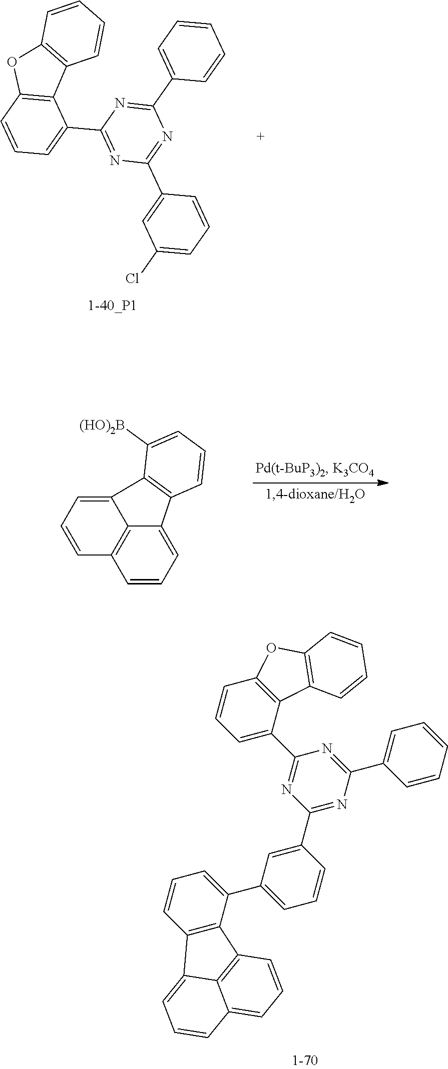 [SMILES: Clc1cccc(-c2nc(-c3ccccc3)nc(-c3cccc4oc5ccccc5c34)n2)c1 ; OB(O)c1cccc2c1-c1cccc3cccc-2c13 ; c1ccc(-c2nc(-c3cccc(-c4cccc5c4-c4cccc6cccc-5c46)c3)nc(-c3cccc4oc5ccccc5c34)n2)cc1]

Compound 1-40_P1 (15 g, 34.6 mmol) and fluoranthen-7-yl boronic acid (8.9 g, 36.3 mmol) were added to 300 mL of 1,4-dioxane, and the mixture was stirred and refluxed. Afterwards, potassium phosphate (14.3 g, 103.7 mmol) was dissolved in 43 ml of water, and then added thereto. Thereafter, it was stirred sufficiently, followed by adding bis(tri-tert-butylphosphine)palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-70 of 13.9 g. (Yield 67%, MS: [M+H]⁺=600).

### Synthesis Example 1-71

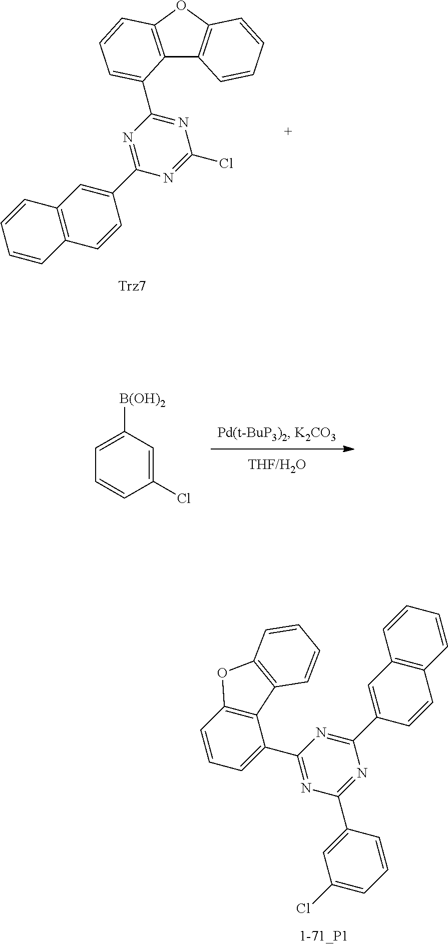 [SMILES: Clc1nc(-c2ccc3ccccc3c2)nc(-c2cccc3oc4ccccc4c23)n1 ; OB(O)c1cccc(Cl)c1 ; Clc1cccc(-c2nc(-c3ccc4ccccc4c3)nc(-c3cccc4oc5ccccc5c34)n2)c1]

### Step 1) Synthesis of Compound 1-71_P1

Compound Trz7 (15 g, 36.8 mmol) and (3-chlorophenyl) boronic acid (6 g, 38.6 mmol) were added to 300 mL of tetrahydrofuran (THF), and the mixture was stirred and refluxed. Afterwards, potassium carbonate (15.2 g, 110.3 mmol) was dissolved in 46 ml of water, and then added thereto. Thereafter, it was stirred sufficiently, followed by adding bis(tri-tert-butylphosphine) palladium (0) (0.2 g, 0.4 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-71_P1 of 13.1 g. (Yield 74%, MS: [M+H]⁺=484).

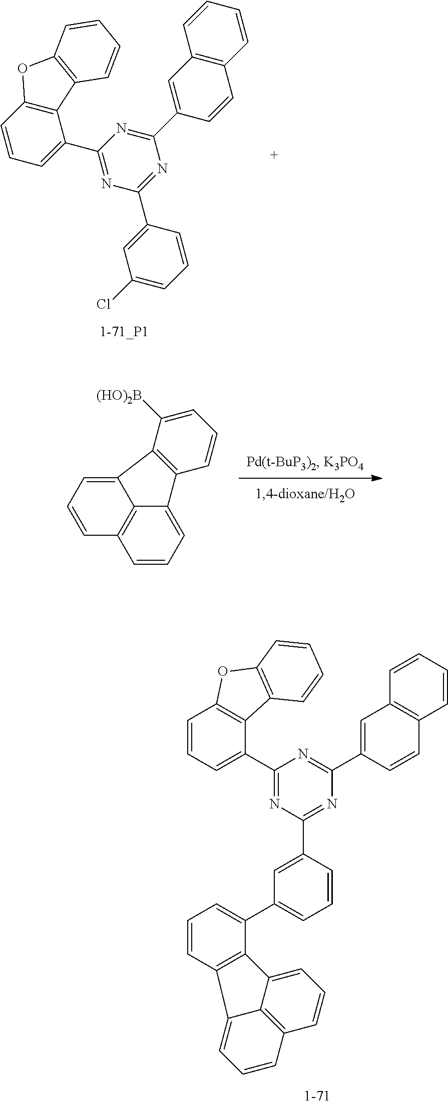 [SMILES: Clc1cccc(-c2nc(-c3ccc4ccccc4c3)nc(-c3cccc4oc5ccccc5c34)n2)c1 ; OB(O)c1cccc2c1-c1cccc3cccc-2c13 ; c1cc(-c2nc(-c3ccc4ccccc4c3)nc(-c3cccc4oc5ccccc5c34)n2)cc(-c2cccc3c2-c2cccc4cccc-3c24)c1]

### Step 2) Synthesis of Compound 1-71

Compound 1-71_P1 (15 g, 31 mmol) prepared in the above Step 1) and fluoranthen-7-yl boronic acid (8 g, 32.5 mmol) were added to 300 ml of 1,4-dioxane, and the mixture was stirred and refluxed. Afterwards, potassium phosphate (19.7 g, 93 mmol) was dissolved in 59 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-71 of 12.3 g. (Yield 61%, MS: [M+H]⁺=650).

### Synthesis Example 1-72

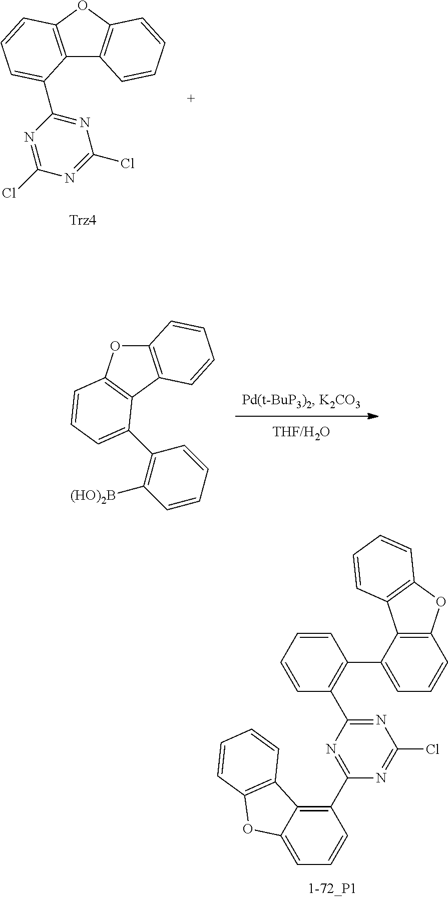 [SMILES: Clc1nc(Cl)nc(-c2cccc3oc4ccccc4c23)n1 ; OB(O)c1ccccc1-c1cccc2oc3ccccc3c12 ; Clc1nc(-c2ccccc2-c2cccc3oc4ccccc4c23)nc(-c2cccc3oc4ccccc4c23)n1]

### Step 1) Synthesis of Compound 1-72_P1

Compound Trz4 (15 g, 47.4 mmol) and (2-(dibenzo[b,d]furan-1-yl)phenyl) boronic acid (14.4 g, 49.8 mmol) were added to 300 mL of tetrahydrofuran (THF), and the mixture was stirred and refluxed. Afterwards, potassium carbonate (19.7 g, 142.3 mmol) was dissolved in 59 mL of water, and then added thereto. Thereafter, it was stirred sufficiently, followed by adding bis(tri-tert-butylphosphine) palladium (0) (0.2 g, 0.5 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-72_P1 of 15.9 g. (Yield 64%, MS: [M+H]⁺=524).

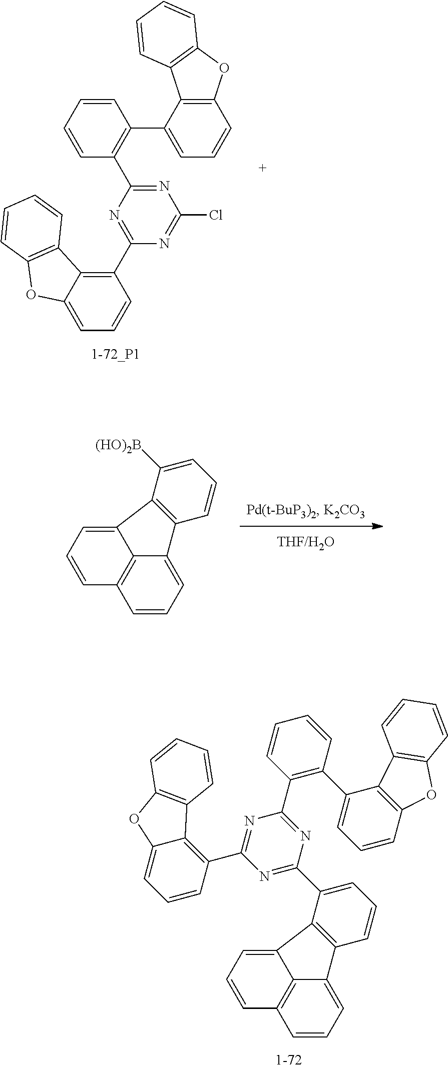 [SMILES: Clc1nc(-c2ccccc2-c2cccc3oc4ccccc4c23)nc(-c2cccc3oc4ccccc4c23)n1 ; OB(O)c1cccc2c1-c1cccc3cccc-2c13 ; c1ccc(-c2cccc3oc4ccccc4c23)c(-c2nc(-c3cccc4c3-c3cccc5cccc-4c35)nc(-c3cccc4oc5ccccc5c34)n2)c1]

### Step 2) Synthesis of Compound 1-72

Compound 1-72_P1 (15 g, 28.6 mmol) prepared in the above Step 1) and fluoranthen-7-yl boronic acid (7.4 g, 30.1 mmol) were added to 300 ml of tetrahydrofuran (THF), and the mixture was stirred and refluxed. Afterwards, potassium carbonate (11.9 g, 85.9 mmol) was dissolved in 36 mL of water, and then added thereto. Thereafter, it was stirred sufficiently, followed by adding bis(tri-tert-butylphosphine) palladium (0) (0.1 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-72 of 12.2 g. (Yield 62%, MS: [M+H]⁺=690).

### Synthesis Example 1-73

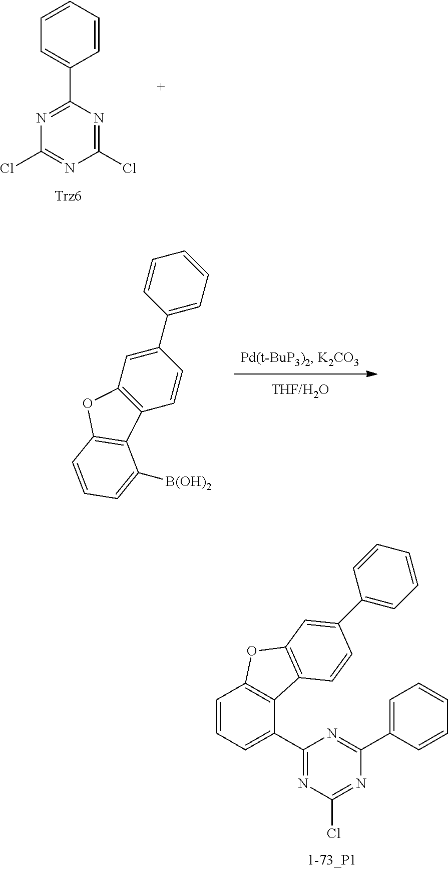 [SMILES: Clc1nc(Cl)nc(-c2ccccc2)n1 ; OB(O)c1cccc2oc3cc(-c4ccccc4)ccc3c12 ; Clc1nc(-c2ccccc2)nc(-c2cccc3oc4cc(-c5ccccc5)ccc4c23)n1]

### Step 1) Synthesis of Compound 1-73_P1

Compound Trz6 (15 g, 66.4 mmol) and (7-phenyldibenzo[b,d]furan-1-yl) boronic acid (20.1 g, 69.7 mmol) were added to 300 mL of tetrahydrofuran (THF), and the mixture was stirred and refluxed. Afterwards, potassium carbonate (27.5 g, 199.1 mmol) was dissolved in 83 mL of water, and then added thereto. Thereafter, it was stirred sufficiently, followed by adding bis(tri-tert-butylphosphine) palladium (0) (0.3 g, 0.7 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-73_P1 of 17.2 g. (Yield 60%, MS: [M+H]⁺=434).

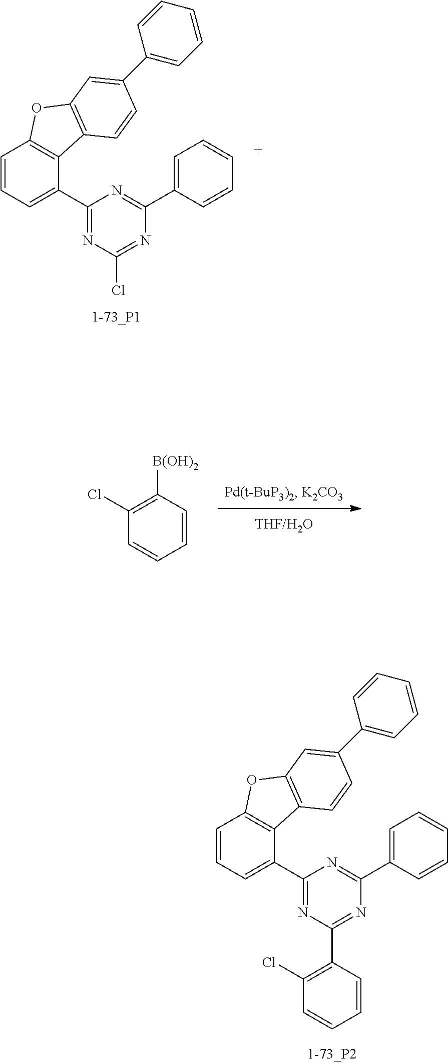 [SMILES: Clc1nc(-c2ccccc2)nc(-c2cccc3oc4cc(-c5ccccc5)ccc4c23)n1 ; OB(O)c1ccccc1Cl ; Clc1ccccc1-c1nc(-c2ccccc2)nc(-c2cccc3oc4cc(-c5ccccc5)ccc4c23)n1]

### Step 2) Synthesis of Compound 1-73_P2

Compound 1-73_P1 (15 g, 34.6 mmol) prepared in the above Step 1) and (2-chlorophenyl) boronic acid (5.7 g, 36.3 mmol) were added to 300 mL of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-73_P2 of 11.3 g. (Yield 64%, MS: [M+H]⁺=510).

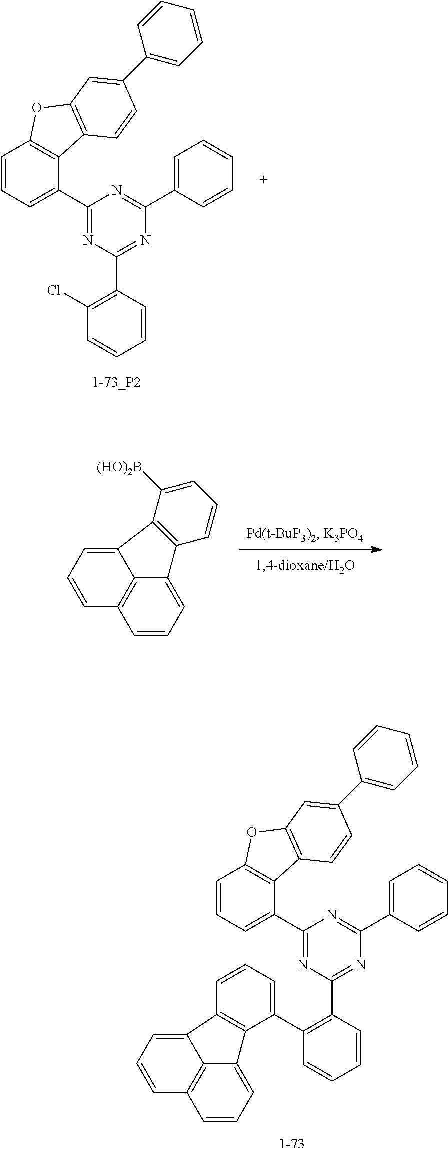 [SMILES: Clc1ccccc1-c1nc(-c2ccccc2)nc(-c2cccc3oc4cc(-c5ccccc5)ccc4c23)n1 ; OB(O)c1cccc2c1-c1cccc3cccc-2c13 ; c1ccc(-c2ccc3c(c2)oc2cccc(-c4nc(-c5ccccc5)nc(-c5ccccc5-c5cccc6c5-c5cccc7cccc-6c57)n4)c23)cc1]

### Step 3) Synthesis of Compound 1-73

Compound 1-73_P2 (15 g, 29.4 mmol) prepared in the above Step 2) and fluoranthen-7-yl boronic acid (7.6 g, 30.9 mmol) were added to 300 mL of 1,4-dioxane, and the mixture was stirred and refluxed. Afterwards, potassium phosphate (18.7 g, 88.2 mmol) was dissolved in 56 mL of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-73 of 13.7 g. (Yield 69%, MS: [M+H]⁺=676).

### Synthesis Example 1-74

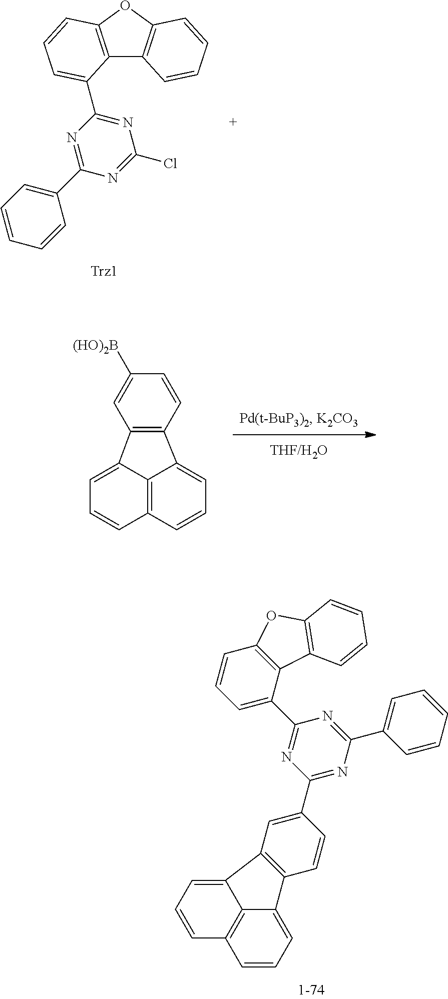 [SMILES: Clc1nc(-c2ccccc2)nc(-c2cccc3oc4ccccc4c23)n1 ; OB(O)c1ccc2c(c1)-c1cccc3cccc-2c13 ; c1ccc(-c2nc(-c3ccc4c(c3)-c3cccc5cccc-4c35)nc(-c3cccc4oc5ccccc5c34)n2)cc1]

Compound Trz1 (15 g, 41.9 mmol) and fluoranthen-8-yl boronic acid (10.8 g, 44 mmol) were added to 300 mL of tetrahydrofuran (THF), and the mixture was stirred and refluxed. Afterwards, potassium carbonate (17.4 g, 125.8 mmol) was dissolved in 52 ml of water, and then added thereto. Thereafter, it was stirred sufficiently, followed by adding bis(tri-tert-butylphosphine) palladium (0) (0.2 g, 0.4 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-74 of 15.8 g. (Yield 72%, MS: [M+H]⁺=524).

### Synthesis Example 1-75

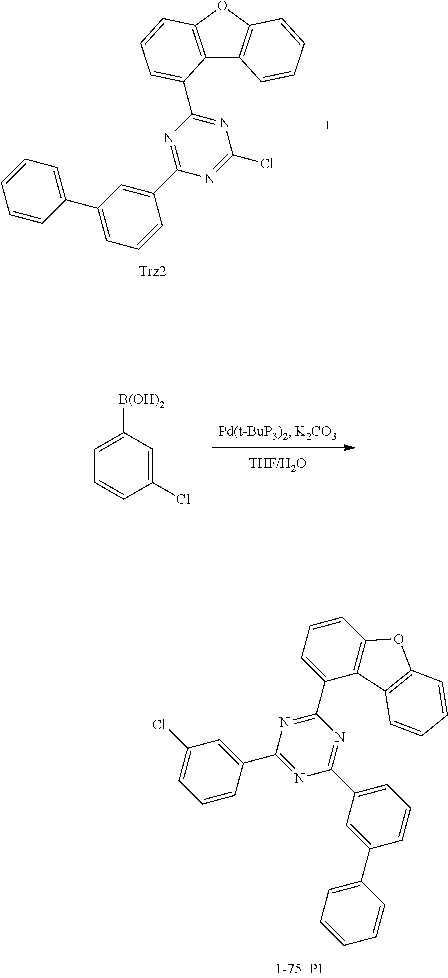 [SMILES: Clc1nc(-c2cccc(-c3ccccc3)c2)nc(-c2cccc3oc4ccccc4c23)n1 ; OB(O)c1cccc(Cl)c1 ; Clc1cccc(-c2nc(-c3cccc(-c4ccccc4)c3)nc(-c3cccc4oc5ccccc5c34)n2)c1]

### Step 1) Synthesis of Compound 1-75_P1

Compound Trz2 (15 g, 34.6 mmol) and (3-chlorophenyl) boronic acid (5.7 g, 36.3 mmol) were added to 300 mL of tetrahydrofuran (THF), and the mixture was stirred and refluxed. Afterwards, potassium carbonate (14.3 g, 103.7 mmol) was dissolved in 43 mL of water, and then added thereto. Thereafter, it was stirred sufficiently, followed by adding bis(tri-tert-butylphosphine) palladium (0) (0.2 g, 0.3 mmol). After a reaction time of 2 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-75_P1 of 11.3 g. (Yield 64%, MS: [M+H]⁺=510).

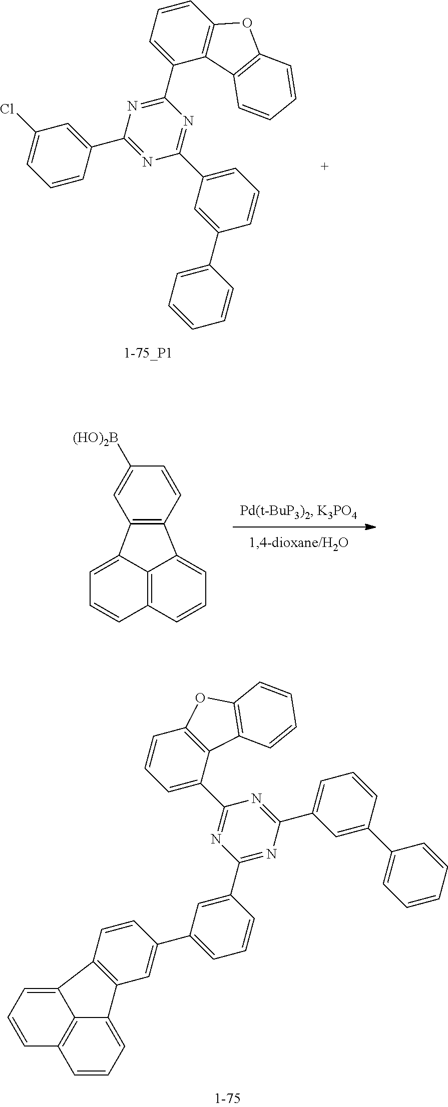 [SMILES: Clc1cccc(-c2nc(-c3cccc(-c4ccccc4)c3)nc(-c3cccc4oc5ccccc5c34)n2)c1 ; OB(O)c1ccc2c(c1)-c1cccc3cccc-2c13 ; c1ccc(-c2cccc(-c3nc(-c4cccc(-c5ccc6c(c5)-c5cccc7cccc-6c57)c4)nc(-c4cccc5oc6ccccc6c45)n3)c2)cc1]

### Step 2) Synthesis of Compound 1-75

Compound 1-75_P1 (15 g, 34.6 mmol) prepared in the above Step 1) and fluoranthen-8-yl boronic acid (8.9 g, 36.3 mmol) were added to 300 mL of 1,4-dioxane, and the mixture was stirred and refluxed. Afterwards, potassium phosphate (22 g, 103.7 mmol) was dissolved in 66 mL of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-75 of 16.6 g. (Yield 71%, MS: [M+H]⁺=676).

### Synthesis Example 1-76

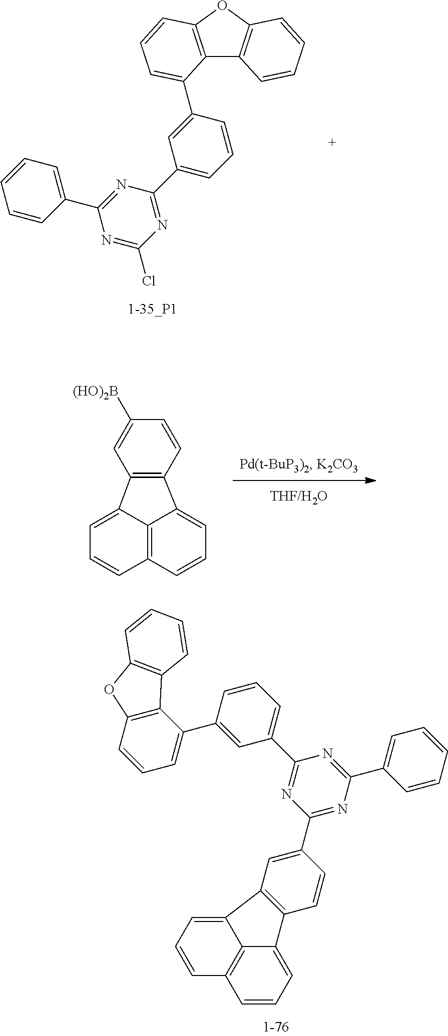

Compound 1-35_P1 (15 g, 34.6 mmol) and fluoranthen-8-yl boronic acid (8.9 g, 36.3 mmol) were added to 300 mL of tetrahydrofuran (THF), and the mixture was stirred and refluxed. Afterwards, potassium carbonate (14.3 g, 103.7 mmol) was dissolved in 43 ml of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-76 of 13.3 g. (Yield 64%, MS: [M+H]⁺=600).

### Synthesis Example 1-77

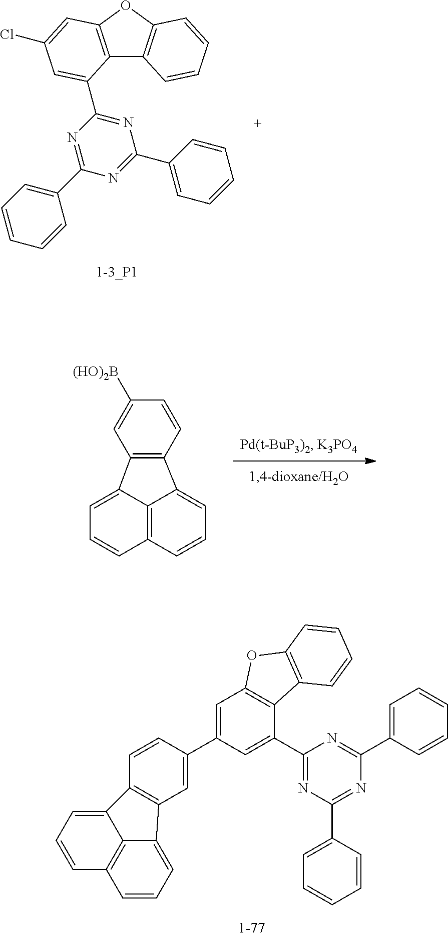 [SMILES: Clc1cc(-c2nc(-c3ccccc3)nc(-c3ccccc3)n2)c2c(c1)oc1ccccc12 ; OB(O)c1ccc2c(c1)-c1cccc3cccc-2c13 ; c1ccc(-c2nc(-c3ccccc3)nc(-c3cc(-c4ccc5c(c4)-c4cccc6cccc-5c46)cc4oc5ccccc5c34)n2)cc1]

Compound 1-3_P1 (15 g, 34.6 mmol) and fluoranthen-8-yl boronic acid (8.9 g, 36.3 mmol) were added to 300 mL of 1,4-dioxane, and the mixture was stirred and refluxed. Afterwards, potassium phosphate (22 g, 103.7 mmol) was dissolved in 66 ml of water, and then added thereto. Thereafter, it was stirred sufficiently, followed by adding bis(tri-tert-butylphosphine) palladium (0) (0.2 g, 0.3 mmol). After a reaction time of 5 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-77 of 13.7 g. (Yield 66%, MS: [M+H]⁺=600).

### Synthesis Example 1-78

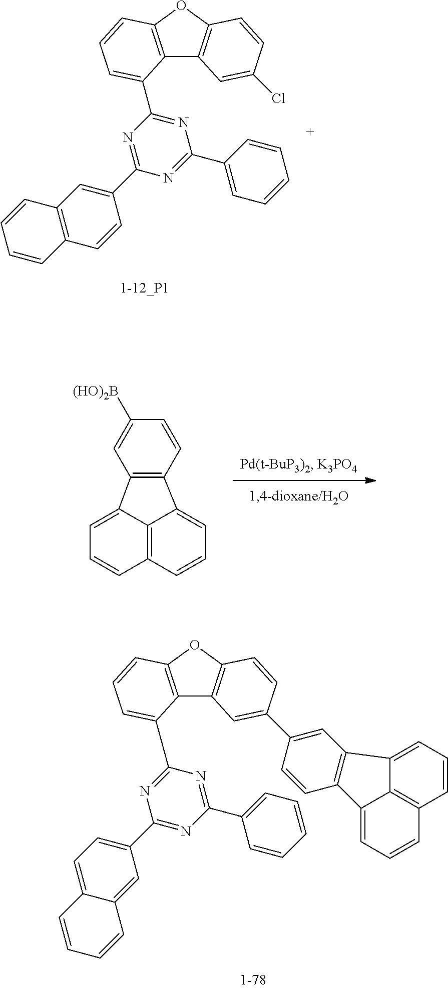 [SMILES: Clc1ccc2oc3cccc(-c4nc(-c5ccccc5)nc(-c5ccc6ccccc6c5)n4)c3c2c1 ; OB(O)c1ccc2c(c1)-c1cccc3cccc-2c13 ; c1ccc(-c2nc(-c3ccc4ccccc4c3)nc(-c3cccc4oc5ccc(-c6ccc7c(c6)-c6cccc8cccc-7c68)cc5c34)n2)cc1]

Compound 1-12_P1 (15 g, 31 mmol) and fluoranthen-8-yl boronic acid (8 g, 32.5 mmol) were added to 300 mL of 1,4-dioxane, and the mixture was stirred and refluxed. Afterwards, potassium phosphate (19.7 g, 93 mmol) was dissolved in 59 ml of water, and then added thereto. Thereafter, it was stirred sufficiently, followed by adding bis(tri-tert-butylphosphine) palladium (0) (0.2 g, 0.3 mmol). After a reaction time of 3 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-78 of 12.9 g. (Yield 64%, MS: [M+H]⁺=650).

### Synthesis Example 1-79

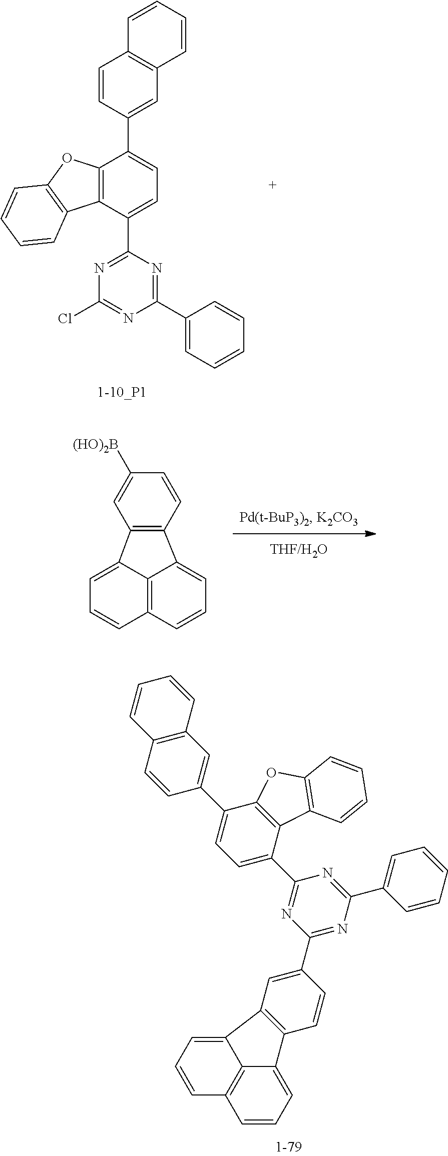 [SMILES: Clc1nc(-c2ccccc2)nc(-c2ccc(-c3ccc4ccccc4c3)c3oc4ccccc4c23)n1 ; OB(O)c1ccc2c(c1)-c1cccc3cccc-2c13 ; c1ccc(-c2nc(-c3ccc4c(c3)-c3cccc5cccc-4c35)nc(-c3ccc(-c4ccc5ccccc5c4)c4oc5ccccc5c34)n2)cc1]

Compound 1-10_P1 (15 g, 31 mmol) and fluoranthen-8-yl boronic acid (8 g, 32.5 mmol) were added to 300 mL of tetrahydrofuran (THF), and the mixture was stirred and refluxed. Afterwards, potassium carbonate (12.9 g, 93 mmol) was dissolved in 39 ml of water, and then added thereto. Thereafter, it was stirred sufficiently, followed by adding bis(tri-tert-butylphosphine) palladium (0) (0.2 g, 0.3 mmol). After a reaction time of 4 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 1-79 of 15.1 g. (Yield 75%, MS: [M+H]⁺=650).

### (Preparation of Second Compound)

### Synthesis Example 2-1

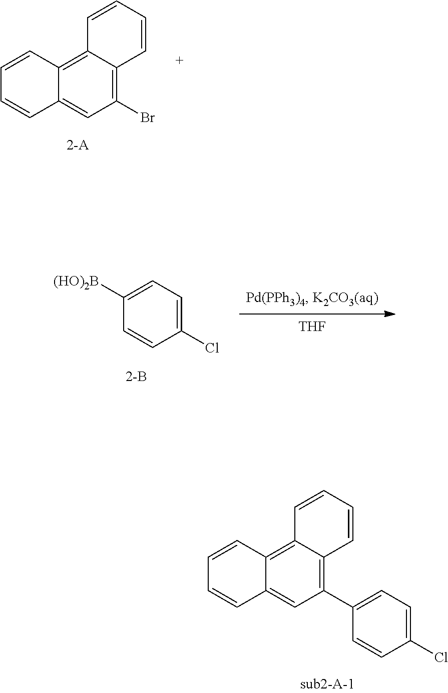 [SMILES: Brc1cc2ccccc2c2ccccc12 ; OB(O)c1ccc(Cl)cc1 ; "THF" ; Clc1ccc(-c2cc3ccccc3c3ccccc23)cc1]

Step 1) Synthesis of Compound sub2-A-1

Under a nitrogen atmosphere, Compound 2-A (15 g, 58.3 mmol) and Compound 2-B (10 g, 64.2 mmol) were added to 300 mL of tetrahydrofuran (THF), and the mixture was stirred and refluxed. Afterwards, potassium carbonate (K₂CO₃, 16.1 g, 116.7 mmol) was dissolved in 48 ml of water, and then added thereto. Thereafter, it was stirred sufficiently, followed by adding Tetrakis(triphenylphosphine) palladium (0) (Pd(PPh₃)₄, 1.3 g, 1.2 mmol). After a reaction time of 11 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-A-1 of 12.6 g. (Yield 75%, MS: [M+H]⁺=289).

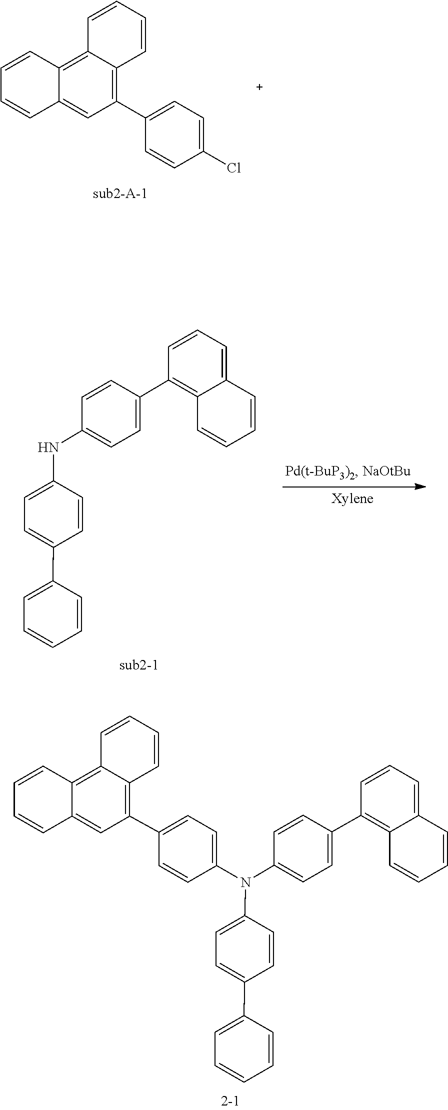 [SMILES: Clc1ccc(-c2cc3ccccc3c3ccccc23)cc1 ; c1ccc(-c2ccc(Nc3ccc(-c4cccc5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1]

### Step 2) Synthesis of Compound 2-1

Under a nitrogen atmosphere, Compound sub2-A-1 (10 g, 34.6 mmol) prepared in the above Step 1), Compound sub2-1 (12.9 g, 34.6 mmol), and sodium tert-butoxide (NaOtBu, 4.3 g, 45 mmol) were added to 200 mL of xylene, and the mixture was stirred and refluxed. Thereafter, bis(tri-tert-butylphosphine) palladium (0) (Pd(t-BuP₃)₂, 0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-1 of 12.7 g. (Yield 59%, MS: [M+H]⁺=624).

### Synthesis Example 2-2

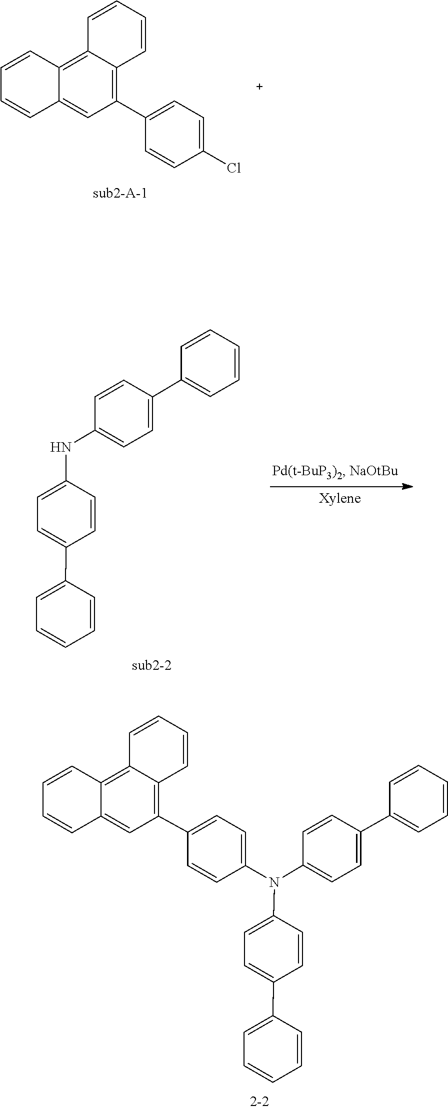 [SMILES: Clc1ccc(-c2cc3ccccc3c3ccccc23)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cc5ccccc5c5ccccc45)cc3)cc2)cc1]

Under a nitrogen atmosphere, Compound sub2-A-1 (10 g, 34.6 mmol), Compound sub2-2 (11.1 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-2 of 10.1 g. (Yield 51%, MS: [M+H]⁺=574).

### Synthesis Example 2-3

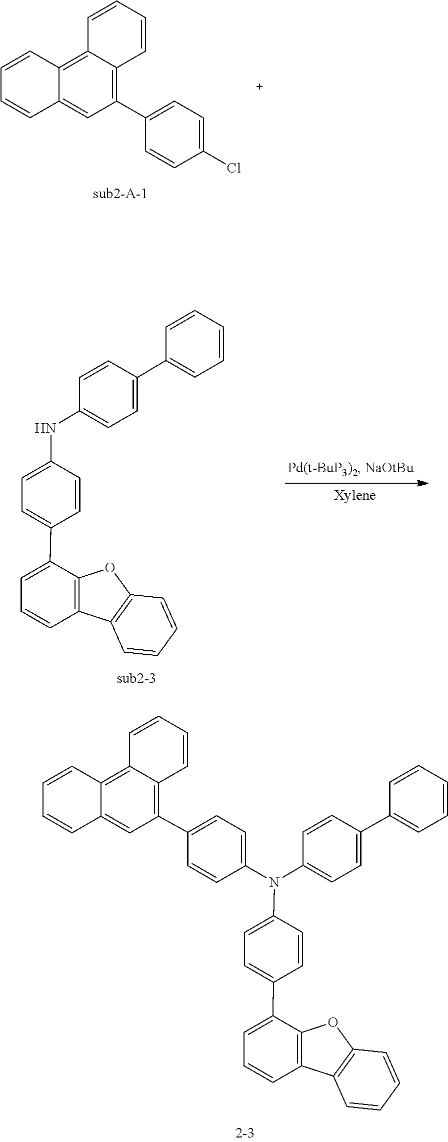 [SMILES: Clc1ccc(-c2cc3ccccc3c3ccccc23)cc1 ; c1ccc(-c2ccc(Nc3ccc(-c4cccc5c4oc4ccccc45)cc3)cc2)cc1 ; c1ccc(-c2ccc(N(c3ccc(-c4cc5ccccc5c5ccccc45)cc3)c3ccc(-c4cccc5c4oc4ccccc45)cc3)cc2)cc1]

Under a nitrogen atmosphere, Compound sub2-A-1 (10 g, 34.6 mmol), Compound sub2-3 (14.3 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-3 of 12.2 g. (Yield 53%, MS: [M+H]⁺=664).

### Synthesis Example 2-4

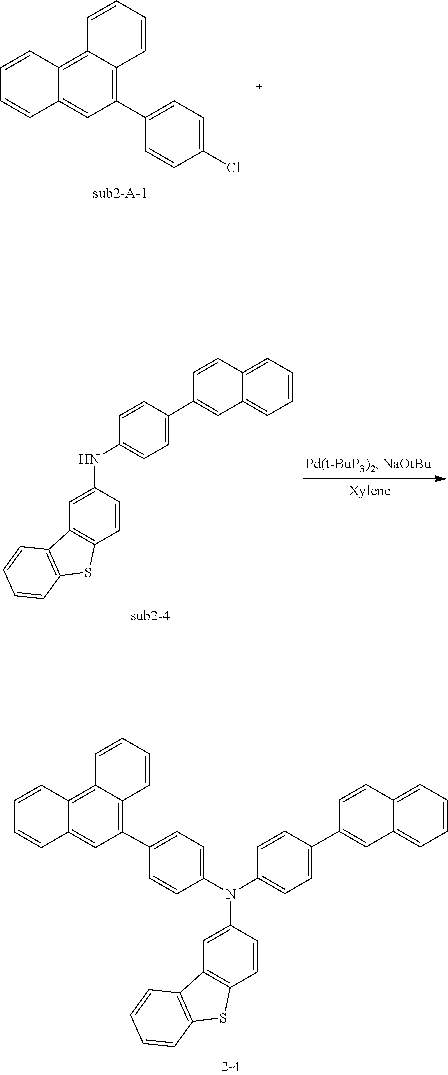 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Under a nitrogen atmosphere, Compound sub2-A-1 (10 g, 34.6 mmol), Compound sub2-4 (13.9 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-4 of 14 g. (Yield 62%, MS: [M+H]⁺=654).

### Synthesis Example 2-5

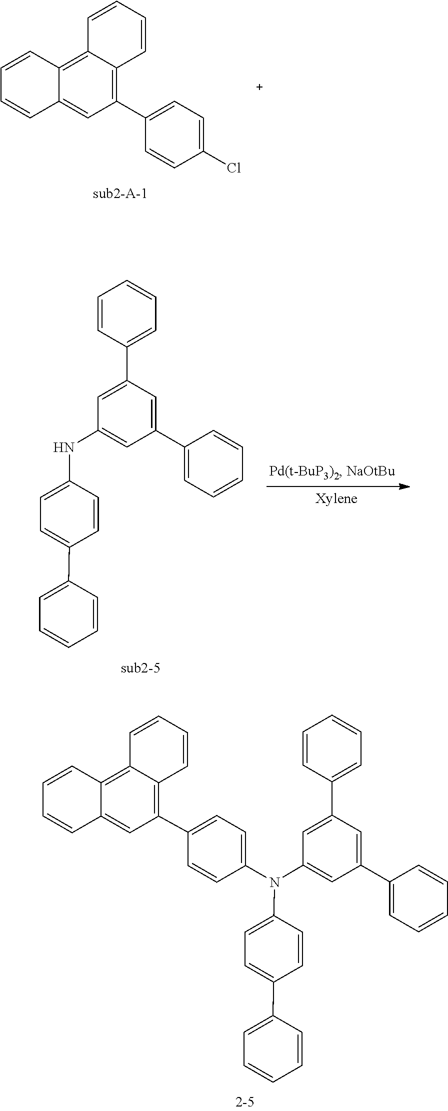 [SMILES: Clc1ccc(-c2cc3ccccc3c3ccccc23)cc1 ; c1ccc(-c2ccc(Nc3cc(-c4ccccc4)cc(-c4ccccc4)c3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1]

Under a nitrogen atmosphere, Compound sub2-A-1 (10 g, 34.6 mmol), Compound sub2-5 (13.8 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-5 of 11.2 g. (Yield 50%, MS: [M+H]⁺=650).

### Synthesis Example 2-6

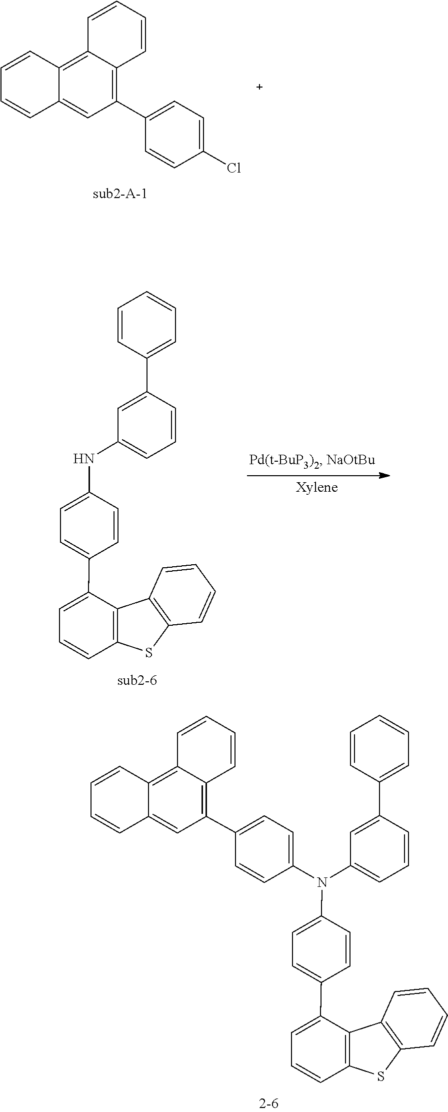 [SMILES: Clc1ccc(-c2cc3ccccc3c3ccccc23)cc1 ; c1ccc(-c2cccc(Nc3ccc(-c4cccc5sc6ccccc6c45)cc3)c2)cc1 ; c1ccc(-c2cccc(N(c3ccc(-c4cc5ccccc5c5ccccc45)cc3)c3ccc(-c4cccc5sc6ccccc6c45)cc3)c2)cc1]

Under a nitrogen atmosphere, Compound sub2-A-1 (10 g, 34.6 mmol), Compound sub2-6 (14.8 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-6 of 12.2 g. (Yield 52%, MS: [M+H]⁺=680).

### Synthesis Example 2-7

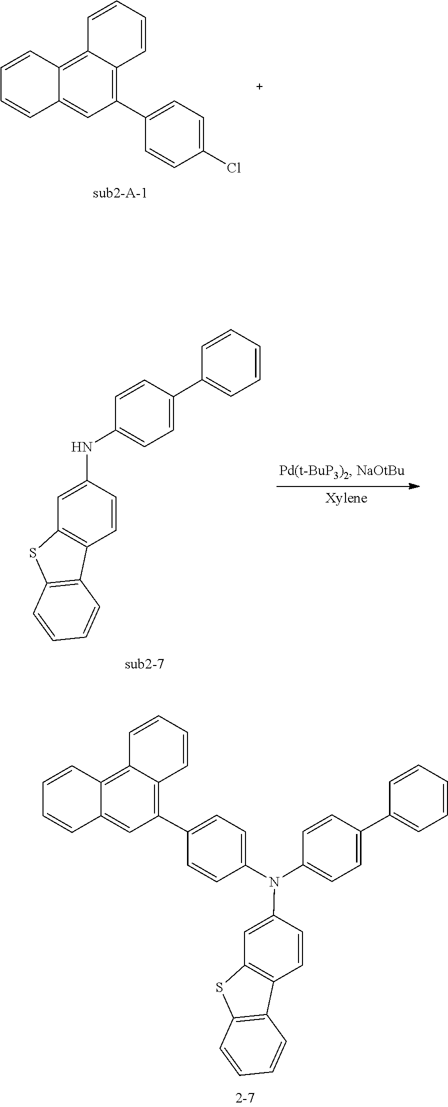 [SMILES: Clc1ccc(-c2cc3ccccc3c3ccccc23)cc1 ; c1ccc(-c2ccc(Nc3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2)cc1]

Under a nitrogen atmosphere, Compound sub2-A-1 (10 g, 34.6 mmol), Compound sub2-7 (12.2 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-7 of 1 g. (Yield 50%, MS: [M+H]⁺=61).

### Synthesis Example 2-8

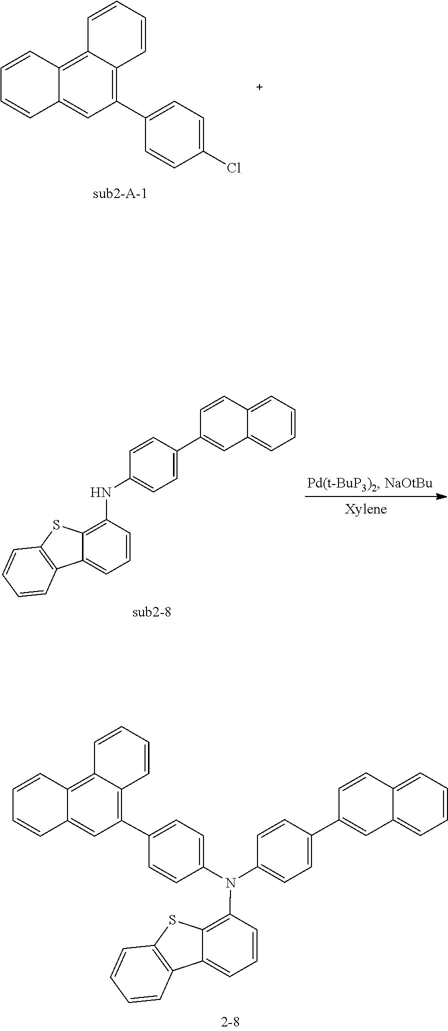 [SMILES: Clc1ccc(-c2cc3ccccc3c3ccccc23)cc1 ; c1ccc2cc(-c3ccc(Nc4cccc5c4sc4ccccc45)cc3)ccc2c1 ; c1ccc2cc(-c3ccc(N(c4ccc(-c5cc6ccccc6c6ccccc56)cc4)c4cccc5c4sc4ccccc45)cc3)ccc2c1]

Under a nitrogen atmosphere, Compound sub2-A-1 (10 g, 34.6 mmol), Compound sub2-8 (13.9 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-8 of 13.3 g. (Yield 59%, MS: [M+H]⁺=654).

### Synthesis Example 2-9

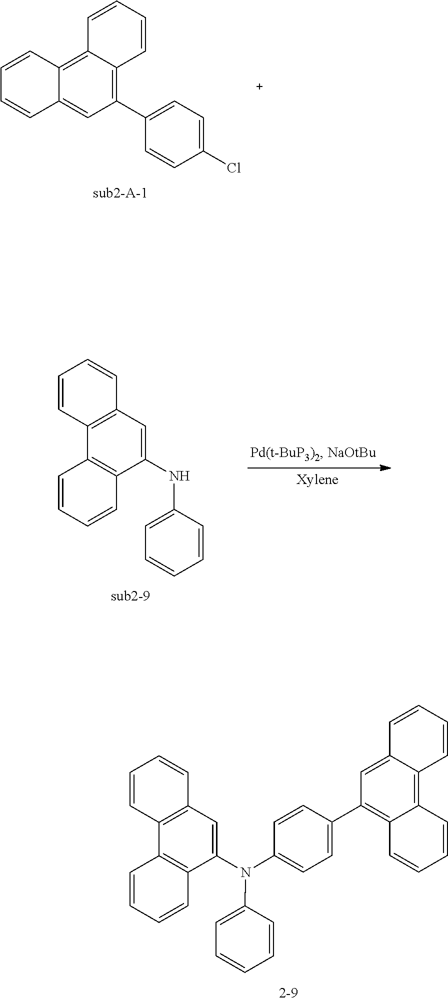 [SMILES: Clc1ccc(-c2cc3ccccc3c3ccccc23)cc1 ; c1ccc(Nc2cc3ccccc3c3ccccc23)cc1 ; c1ccc(N(c2ccc(-c3cc4ccccc4c4ccccc34)cc2)c2cc3ccccc3c3ccccc23)cc1]

Under a nitrogen atmosphere, Compound sub2-A-1 (10 g, 34.6 mmol), Compound sub2-9 (9.3 g, 34.6 mmol), and sodium tert-butoxide (4.3 g, 45 mmol) were added to 200 ml of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-9 of 11.2 g. (Yield 62%, MS: [M+H]⁺=522).

### Synthesis Example 2-10

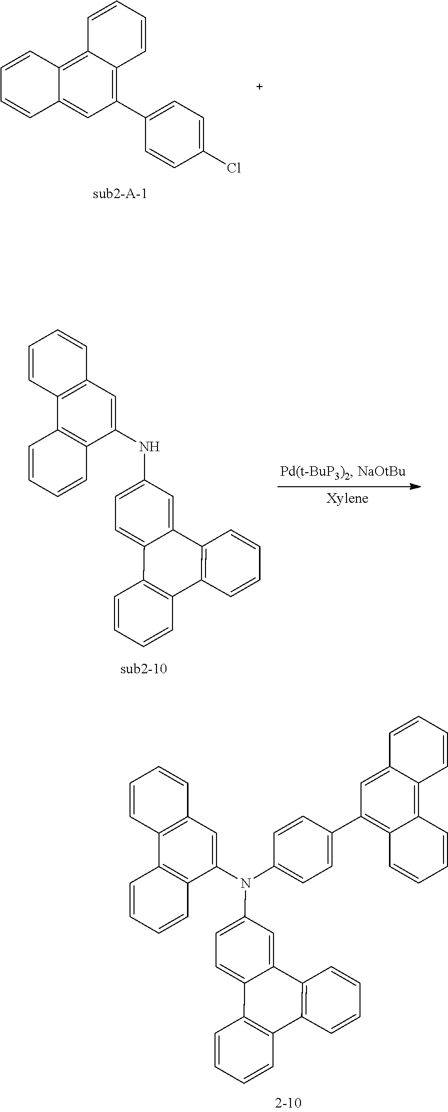 [SMILES: Clc1ccc(-c2cc3ccccc3c3ccccc23)cc1 ; c1ccc2c(c1)cc(Nc1ccc3c4ccccc4c4ccccc4c3c1)c1ccccc12 ; c1ccc2c(c1)cc(-c1ccc(N(c3ccc4c5ccccc5c5ccccc5c4c3)c3cc4ccccc4c4ccccc34)cc1)c1ccccc12]

Under a nitrogen atmosphere, Compound sub2-A-1 (10 g, 34.6 mmol), Compound sub2-10 (14.5 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-10 of 14.4 g. (Yield 62%, MS: [M+H]⁺=672).

### Synthesis Example 2-11

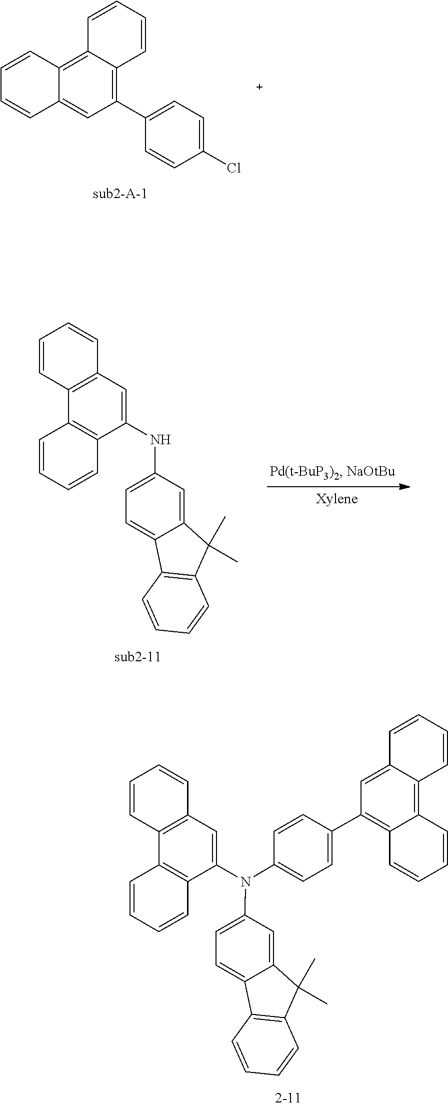 [SMILES: Clc1ccc(-c2cc3ccccc3c3ccccc23)cc1 ; CC1(C)c2ccccc2-c2ccc(Nc3cc4ccccc4c4ccccc34)cc21 ; CC1(C)c2ccccc2-c2ccc(N(c3ccc(-c4cc5ccccc5c5ccccc45)cc3)c3cc4ccccc4c4ccccc34)cc21]

Under a nitrogen atmosphere, Compound sub2-A-1 (10 g, 34.6 mmol), Compound sub2-11 (13.4 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-11 of 12.4 g. (Yield 56%, MS: [M+H]⁺=638).

### Synthesis Example 2-12

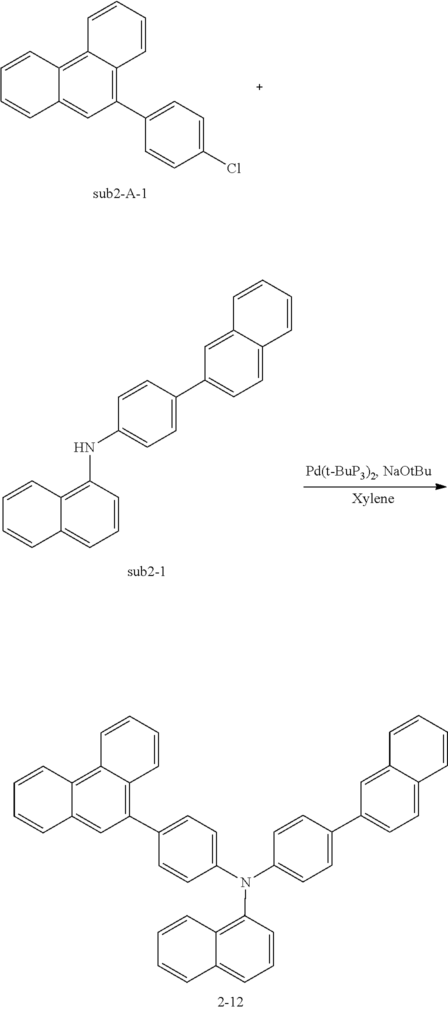 [SMILES: Clc1ccc(-c2cc3ccccc3c3ccccc23)cc1 ; c1ccc2cc(-c3ccc(Nc4cccc5ccccc45)cc3)ccc2c1 ; c1ccc2cc(-c3ccc(N(c4ccc(-c5cc6ccccc6c6ccccc56)cc4)c4cccc5ccccc45)cc3)ccc2c1]

Under a nitrogen atmosphere, Compound sub2-A-1 (10 g, 34.6 mmol), Compound sub2-12 (12 g, 34.6 mmol), and sodium tert-butoxide (4.3 g, 45 mmol) were added to 200 ml of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-12 of 11 g. (Yield 53%, MS: [M+H]⁺=598).

### Synthesis Example 2-13

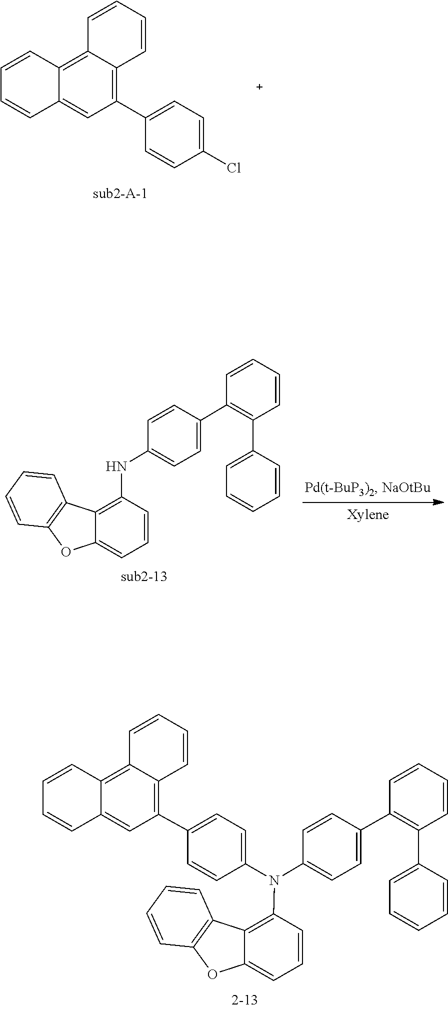 [SMILES: Clc1ccc(-c2cc3ccccc3c3ccccc23)cc1 ; c1ccc(-c2ccccc2-c2ccc(Nc3cccc4oc5ccccc5c34)cc2)cc1 ; c1ccc(-c2ccccc2-c2ccc(N(c3ccc(-c4cc5ccccc5c5ccccc45)cc3)c3cccc4oc5ccccc5c34)cc2)cc1]

Under a nitrogen atmosphere, Compound sub2-A-1 (10 g, 34.6 mmol), Compound sub2-13 (14.3 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-13 of 15.6 g. (Yield 68%, MS: [M+H]⁺=664).

### Synthesis Example 2-14

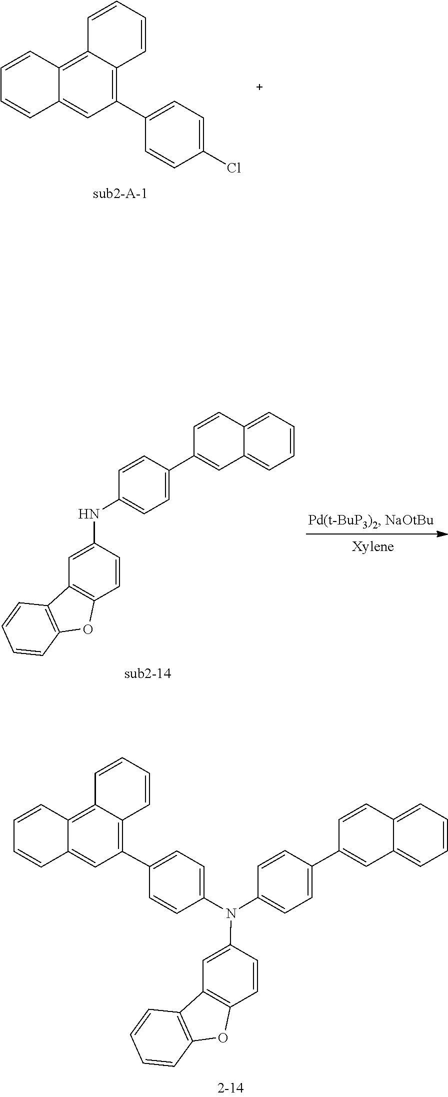 [SMILES: Clc1ccc(-c2cc3ccccc3c3ccccc23)cc1 ; c1ccc2cc(-c3ccc(Nc4ccc5oc6ccccc6c5c4)cc3)ccc2c1 ; c1ccc2cc(-c3ccc(N(c4ccc(-c5cc6ccccc6c6ccccc56)cc4)c4ccc5oc6ccccc6c5c4)cc3)ccc2c1]

Under a nitrogen atmosphere, Compound sub2-A-1 (10 g, 34.6 mmol), Compound sub2-14 (13.3 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-14 of 13.2 g. (Yield 68%, MS: [M+H]⁺=638).

### Synthesis Example 2-15

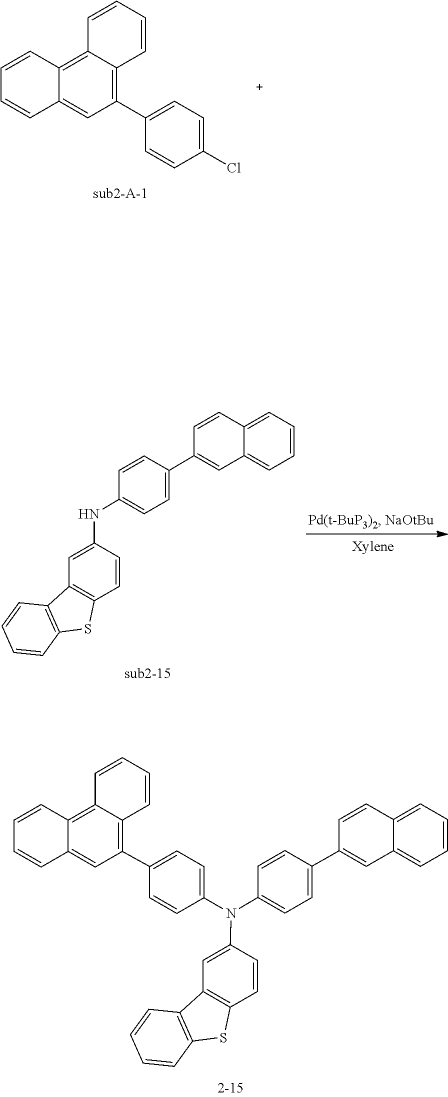 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Under a nitrogen atmosphere, Compound sub2-A-1 (10 g, 34.6 mmol), Compound sub2-15 (13.9 g, 34.6 mmol), and sodium tert-butoxide (3.7 g, 38.1 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-15 of 12 g. (Yield 53%, MS: [M+H]⁺=654).

### Synthesis Example 2-16

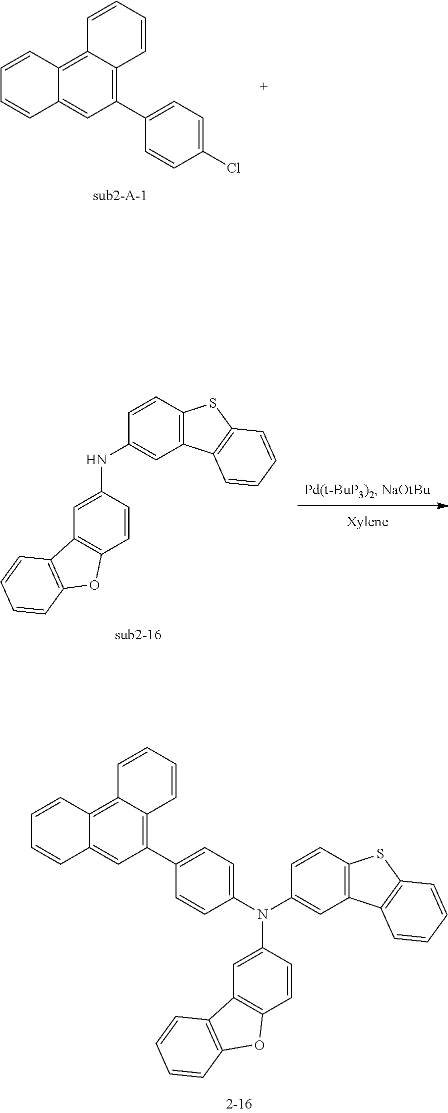 [SMILES: Clc1ccc(-c2cc3ccccc3c3ccccc23)cc1 ; c1ccc2c(c1)oc1ccc(Nc3ccc4sc5ccccc5c4c3)cc12 ; c1ccc2c(c1)cc(-c1ccc(N(c3ccc4oc5ccccc5c4c3)c3ccc4sc5ccccc5c4c3)cc1)c1ccccc12]

Under a nitrogen atmosphere, Compound sub2-A-1 (10 g, 34.6 mmol), Compound sub2-16 (12.7 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-16 of 13.7 g. (Yield 64%, MS: [M+H]⁺=618).

### Synthesis Example 2-17

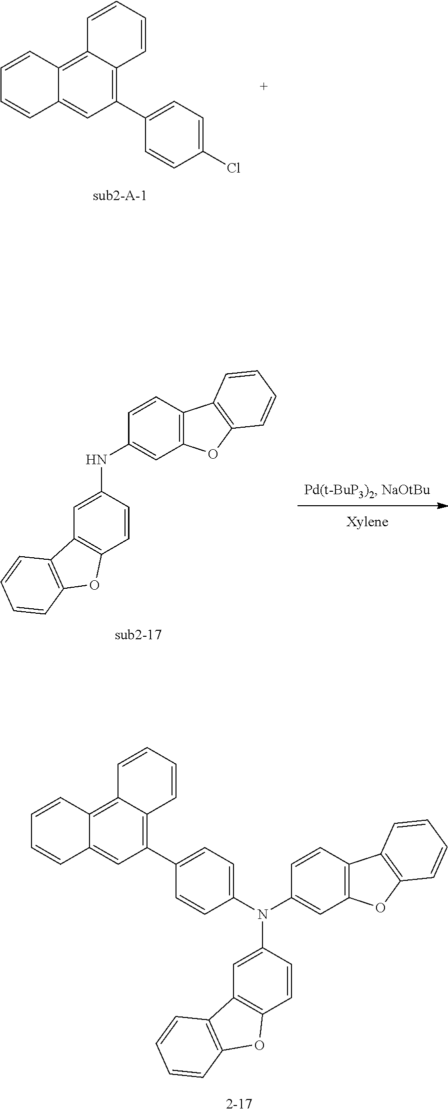 [SMILES: Clc1ccc(-c2cc3ccccc3c3ccccc23)cc1 ; c1ccc2c(c1)oc1cc(Nc3ccc4oc5ccccc5c4c3)ccc12 ; c1ccc2c(c1)cc(-c1ccc(N(c3ccc4c(c3)oc3ccccc34)c3ccc4oc5ccccc5c4c3)cc1)c1ccccc12]

Under a nitrogen atmosphere, Compound sub2-A-1 (10 g, 34.6 mmol), Compound sub2-17 (12.1 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-17 of 11.5 g. (Yield 55%, MS: [M+H]⁺=602).

### Synthesis Example 2-18

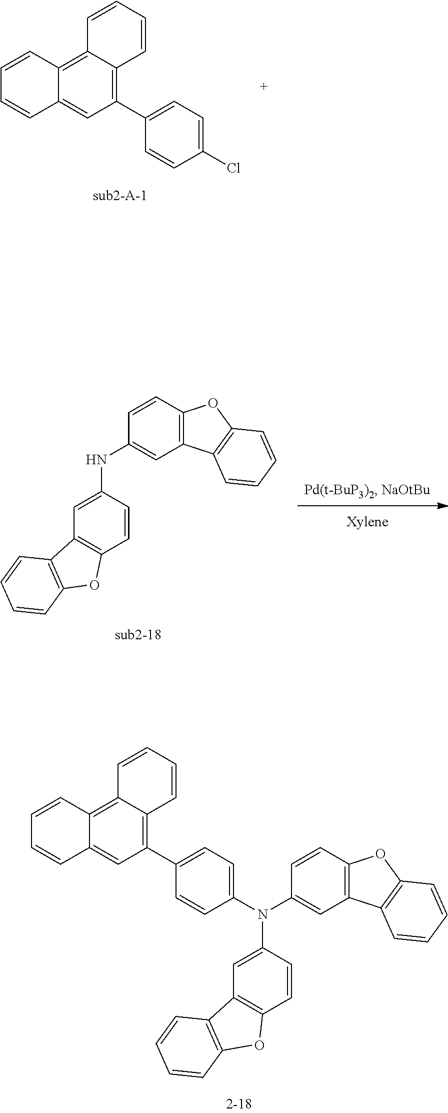 [SMILES: Clc1ccc(-c2cc3ccccc3c3ccccc23)cc1 ; c1ccc2c(c1)oc1ccc(Nc3ccc4oc5ccccc5c4c3)cc12 ; c1ccc2c(c1)cc(-c1ccc(N(c3ccc4oc5ccccc5c4c3)c3ccc4oc5ccccc5c4c3)cc1)c1ccccc12]

Under a nitrogen atmosphere, Compound sub2-A-1 (10 g, 34.6 mmol), Compound sub2-18 (12.1 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-18 of 14.4 g. (Yield 69%, MS: [M+H]⁺=602).

### Synthesis Example 2-19

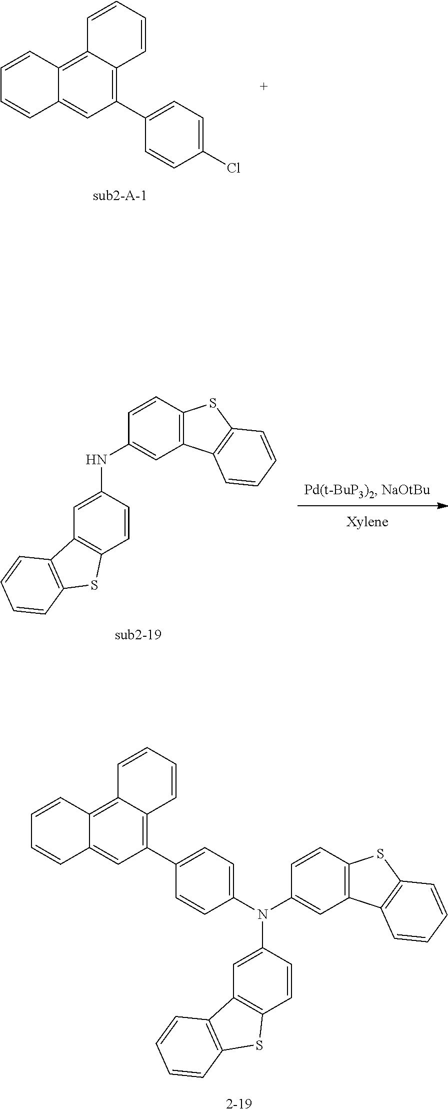 [SMILES: Clc1ccc(-c2cc3ccccc3c3ccccc23)cc1 ; c1ccc2c(c1)sc1ccc(Nc3ccc4sc5ccccc5c4c3)cc12 ; c1ccc2c(c1)cc(-c1ccc(N(c3ccc4sc5ccccc5c4c3)c3ccc4sc5ccccc5c4c3)cc1)c1ccccc12]

Under a nitrogen atmosphere, Compound sub2-A-1 (10 g, 34.6 mmol), Compound sub2-19 (13.2 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-19 of 11.4 g. (Yield 52%, MS: [M+H]⁺=634).

### Synthesis Example 2-20

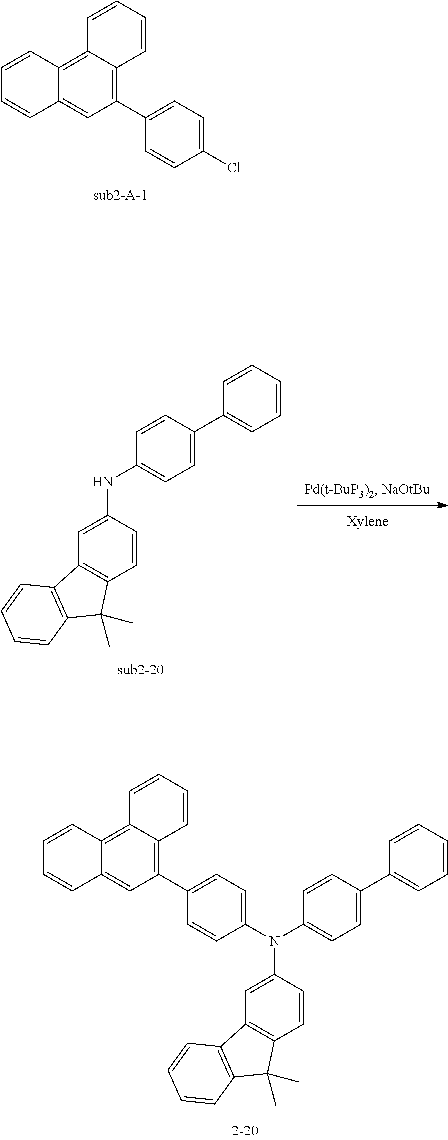 [SMILES: Clc1ccc(-c2cc3ccccc3c3ccccc23)cc1 ; CC1(C)c2ccccc2-c2cc(Nc3ccc(-c4ccccc4)cc3)ccc21 ; CC1(C)c2ccccc2-c2cc(N(c3ccc(-c4ccccc4)cc3)c3ccc(-c4cc5ccccc5c5ccccc45)cc3)ccc21]

Under a nitrogen atmosphere, Compound sub2-A-1 (10 g, 34.6 mmol), Compound sub2-20 (12.5 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-20 of 13.2 g. (Yield 62%, MS: [M+H]⁺=614).

### Synthesis Example 2-21

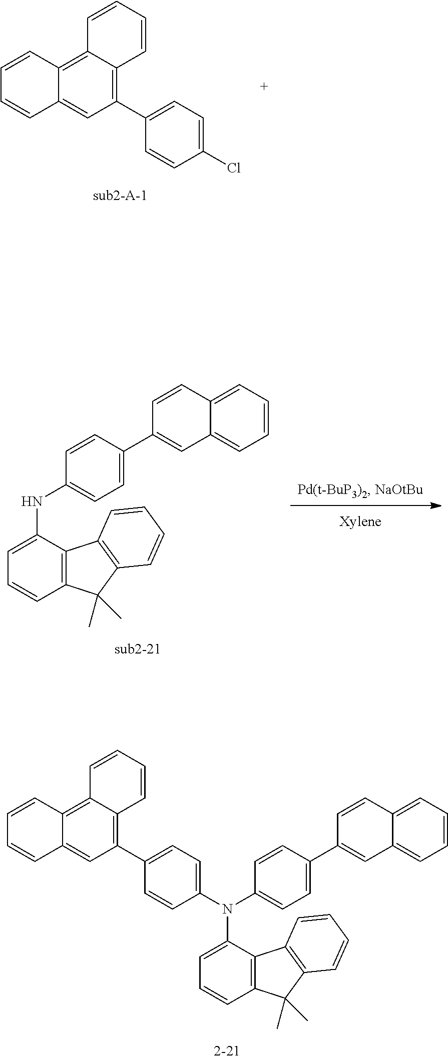 [SMILES: Clc1ccc(-c2cc3ccccc3c3ccccc23)cc1 ; CC1(C)c2ccccc2-c2c(Nc3ccc(-c4ccc5ccccc5c4)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21]

Under a nitrogen atmosphere, Compound sub2-A-1 (10 g, 34.6 mmol), Compound sub2-21 (14.3 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-21 of 14.2 g. (Yield 62%, MS: [M+H]⁺=664).

### Synthesis Example 2-22

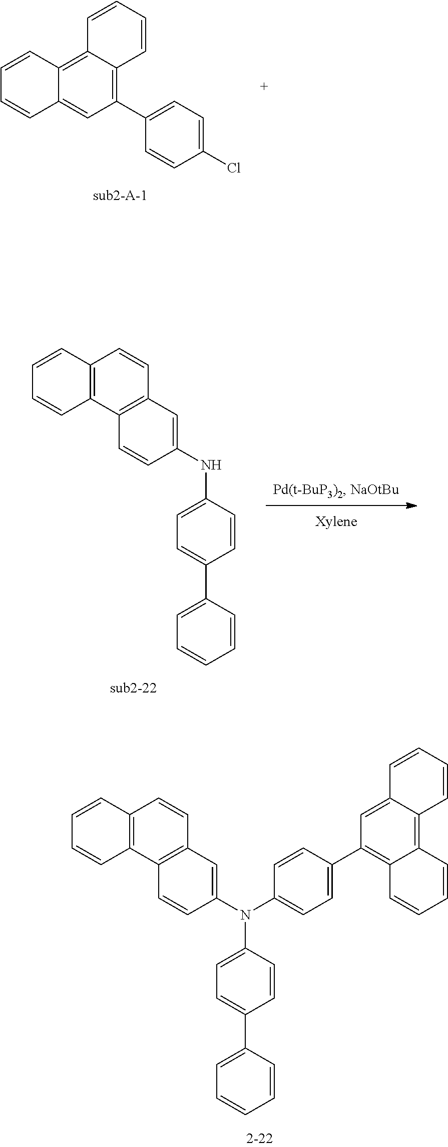 [SMILES: Clc1ccc(-c2cc3ccccc3c3ccccc23)cc1 ; c1ccc(-c2ccc(Nc3ccc4c(ccc5ccccc54)c3)cc2)cc1 ; c1ccc(-c2ccc(N(c3ccc(-c4cc5ccccc5c5ccccc45)cc3)c3ccc4c(ccc5ccccc54)c3)cc2)cc1]

Under a nitrogen atmosphere, Compound sub2-A-1 (10 g, 34.6 mmol), Compound sub2-22 (12 g, 34.6 mmol), and sodium tert-butoxide (4.3 g, 45 mmol) were added to 200 ml of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-22 of 11.2 g. (Yield 54%, MS: [M+H]⁺=598).

### Synthesis Example 2-23

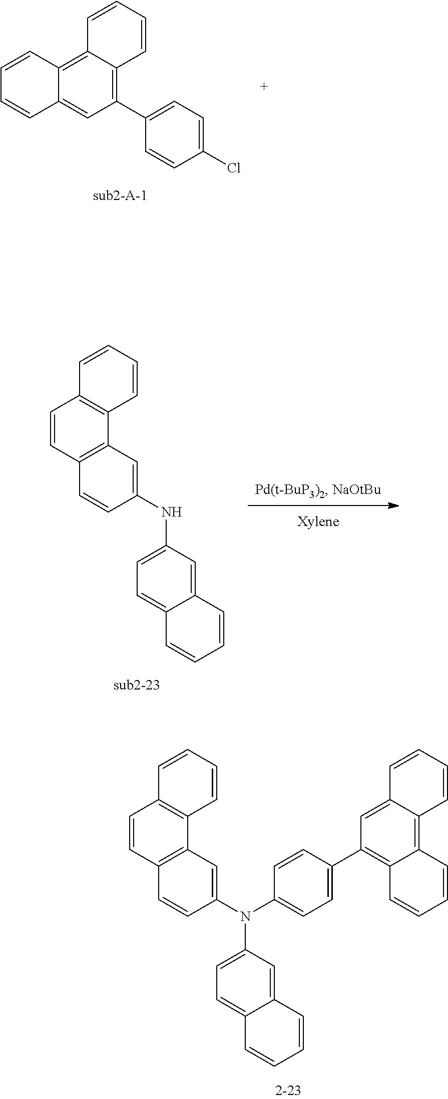 [SMILES: Clc1ccc(-c2cc3ccccc3c3ccccc23)cc1 ; c1ccc2cc(Nc3ccc4ccc5ccccc5c4c3)ccc2c1 ; c1ccc2cc(N(c3ccc(-c4cc5ccccc5c5ccccc45)cc3)c3ccc4ccc5ccccc5c4c3)ccc2c1]

Under a nitrogen atmosphere, Compound sub2-A-1 (10 g, 34.6 mmol), Compound sub2-23 (11.1 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-23 of 11.9 g. (Yield 60%, MS: [M+H]⁺=572).

### Synthesis Example 2-24

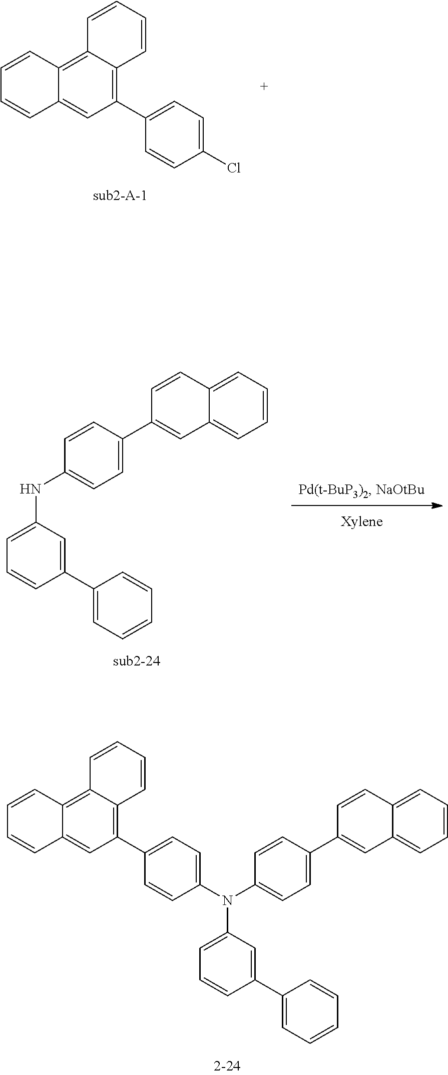 [SMILES: Clc1ccc(-c2cc3ccccc3c3ccccc23)cc1 ; c1ccc(-c2cccc(Nc3ccc(-c4ccc5ccccc5c4)cc3)c2)cc1 ; c1ccc(-c2cccc(N(c3ccc(-c4ccc5ccccc5c4)cc3)c3ccc(-c4cc5ccccc5c5ccccc45)cc3)c2)cc1]

Under a nitrogen atmosphere, Compound sub2-A-1 (10 g, 34.6 mmol), Compound sub2-24 (12.9 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-24 of 13.6 g. (Yield 63%, MS: [M+H]⁺=624).

### Synthesis Example 2-25

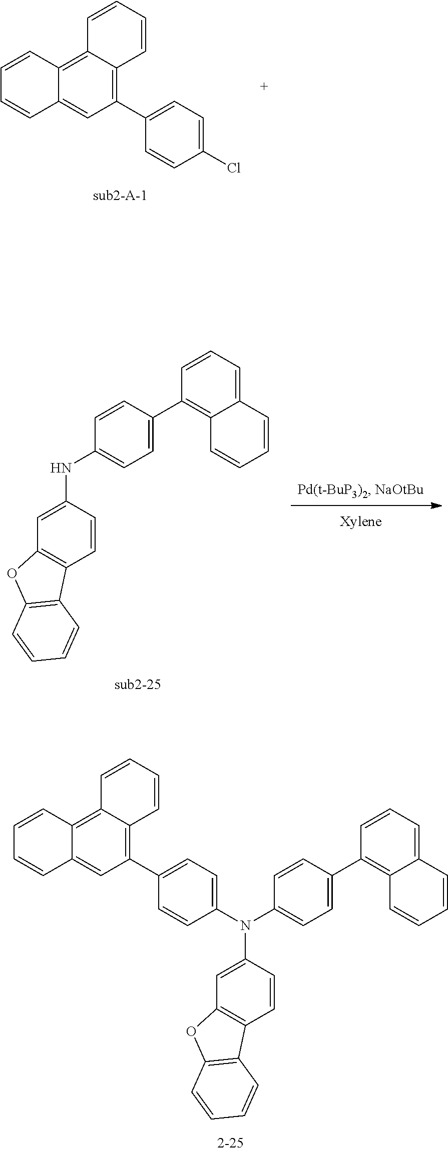 [SMILES: Clc1ccc(-c2cc3ccccc3c3ccccc23)cc1 ; c1ccc2c(-c3ccc(Nc4ccc5c(c4)oc4ccccc45)cc3)cccc2c1 ; c1ccc2c(-c3ccc(N(c4ccc(-c5cc6ccccc6c6ccccc56)cc4)c4ccc5c(c4)oc4ccccc45)cc3)cccc2c1]

Under a nitrogen atmosphere, Compound sub2-A-1 (10 g, 34.6 mmol), Compound sub2-25 (13.3 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-25 of 14.3 g. (Yield 65%, MS: [M+H]⁺=638).

### Synthesis Example 2-26

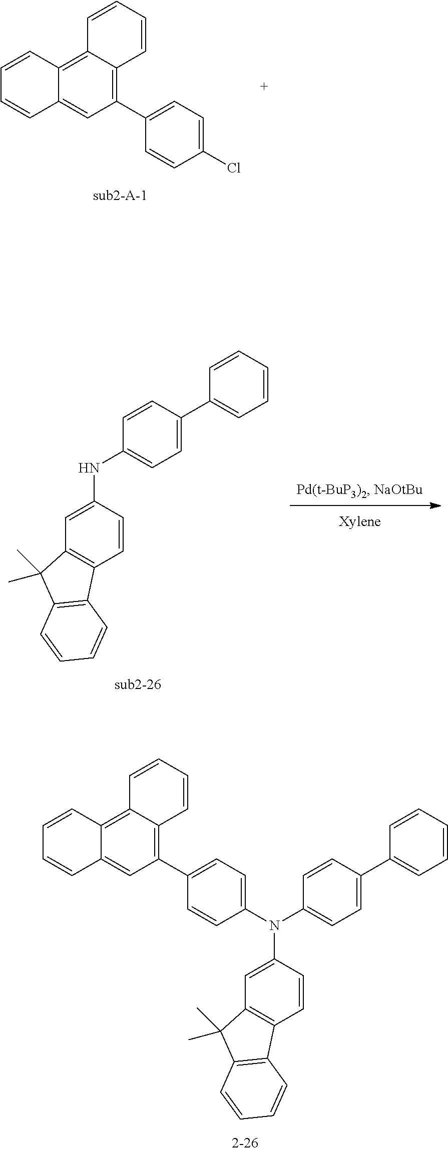 [SMILES: Clc1ccc(-c2cc3ccccc3c3ccccc23)cc1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4cc5ccccc5c5ccccc45)cc3)cc21]

Under a nitrogen atmosphere, Compound sub2-A-1 (10 g, 34.6 mmol), Compound sub2-26 (12.5 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-26 of 10.8 g. (Yield 51%, MS: [M+H]⁺=614).

### Synthesis Example 2-27

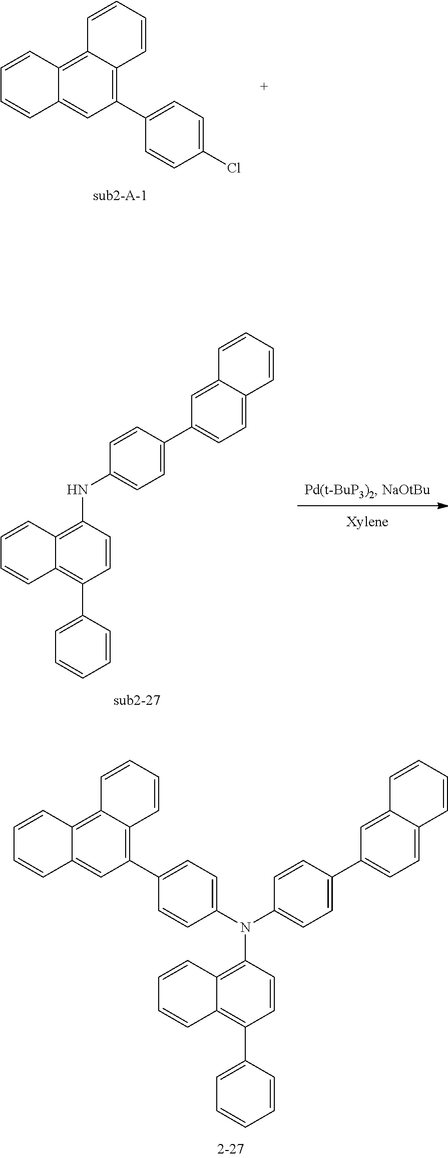 [SMILES: Clc1ccc(-c2cc3ccccc3c3ccccc23)cc1 ; c1ccc(-c2ccc(Nc3ccc(-c4ccc5ccccc5c4)cc3)c3ccccc23)cc1 ; c1ccc(-c2ccc(N(c3ccc(-c4ccc5ccccc5c4)cc3)c3ccc(-c4cc5ccccc5c5ccccc45)cc3)c3ccccc23)cc1]

Under a nitrogen atmosphere, Compound sub2-A-1 (10 g, 34.6 mmol), Compound sub2-27 (14.6 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-27 of 16.1 g. (Yield 69%, MS: [M+H]⁺=674).

### Synthesis Example 2-28

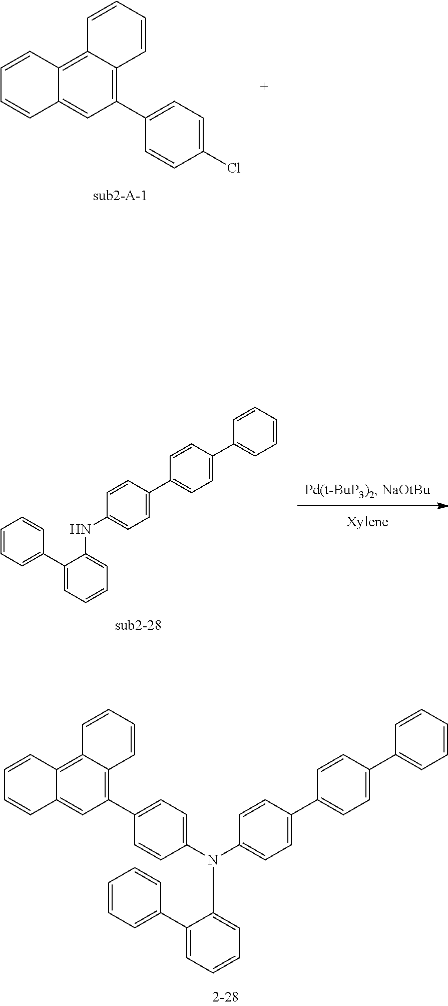 [SMILES: Clc1ccc(-c2cc3ccccc3c3ccccc23)cc1 ; c1ccc(-c2ccc(-c3ccc(Nc4ccccc4-c4ccccc4)cc3)cc2)cc1 ; c1ccc(-c2ccc(-c3ccc(N(c4ccc(-c5cc6ccccc6c6ccccc56)cc4)c4ccccc4-c4ccccc4)cc3)cc2)cc1]

Under a nitrogen atmosphere, Compound sub2-A-1 (10 g, 34.6 mmol), Compound sub2-28 (13.8 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-28 of 11.2 g. (Yield 50%, MS: [M+H]⁺=650).

### Synthesis Example 2-29

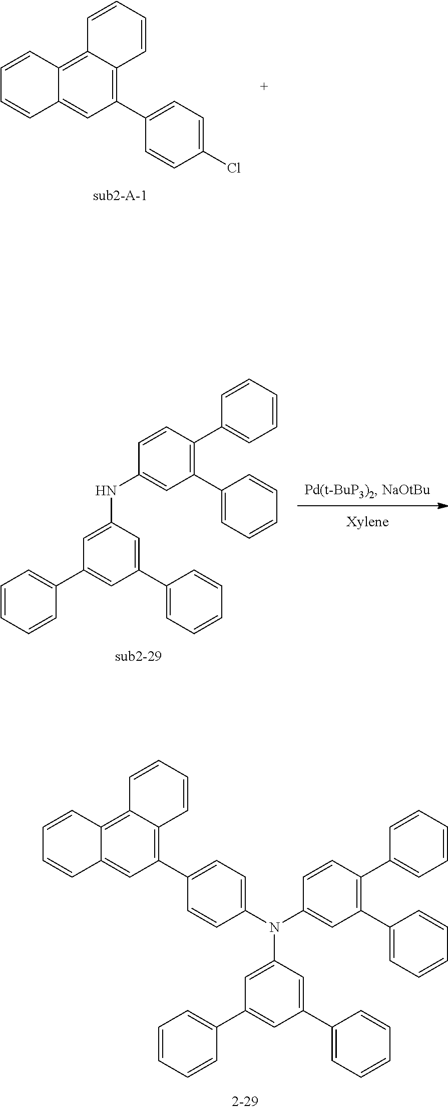 [SMILES: Clc1ccc(-c2cc3ccccc3c3ccccc23)cc1 ; c1ccc(-c2cc(Nc3ccc(-c4ccccc4)c(-c4ccccc4)c3)cc(-c3ccccc3)c2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1]

Under a nitrogen atmosphere, Compound sub2-A-1 (10 g, 34.6 mmol), Compound sub2-29 (16.4 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-29 of 17.1 g. (Yield 68%, MS: [M+H]⁺=726).

### Synthesis Example 2-30

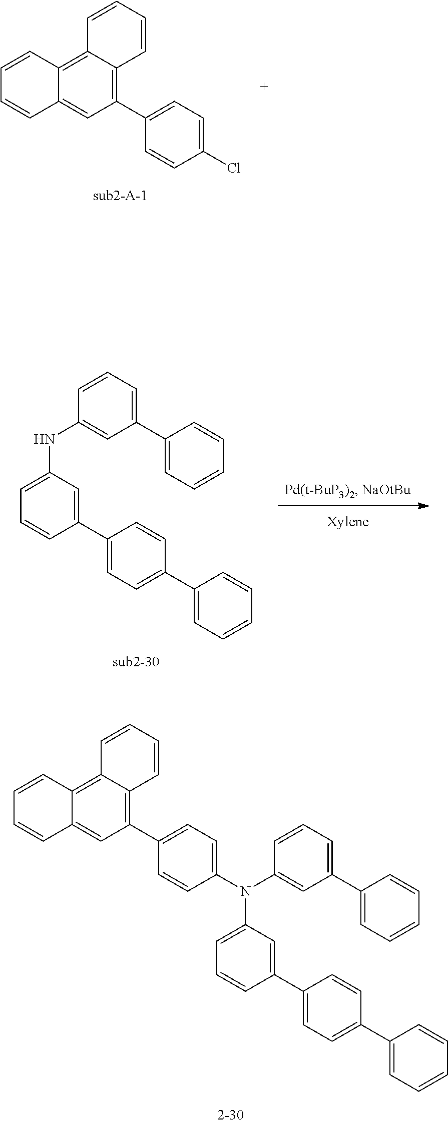 [SMILES: Clc1ccc(-c2cc3ccccc3c3ccccc23)cc1 ; c1ccc(-c2ccc(-c3cccc(Nc4cccc(-c5ccccc5)c4)c3)cc2)cc1 ; c1ccc(-c2ccc(-c3cccc(N(c4ccc(-c5cc6ccccc6c6ccccc56)cc4)c4cccc(-c5ccccc5)c4)c3)cc2)cc1]

Under a nitrogen atmosphere, Compound sub2-A-1 (10 g, 34.6 mmol), Compound sub2-30 (13.8 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-30 of 14.4 g. (Yield 64%, MS: [M+H]⁺=650).

### Synthesis Example 2-31

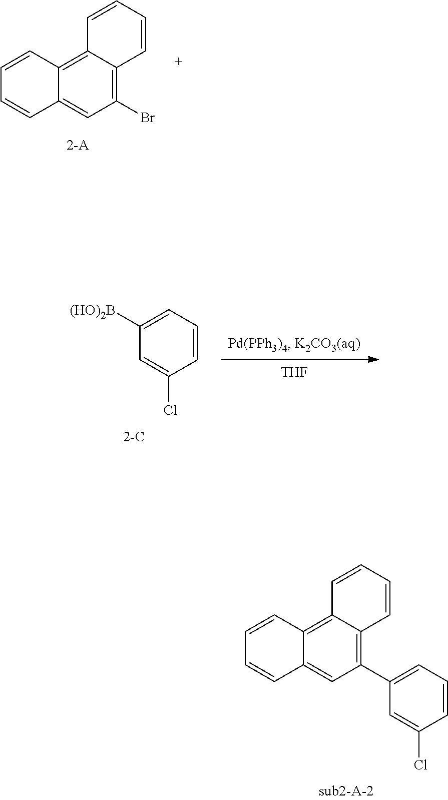 [SMILES: Brc1cc2ccccc2c2ccccc12 ; OB(O)c1cccc(Cl)c1 ; "THF" ; Clc1cccc(-c2cc3ccccc3c3ccccc23)c1]

Step 1) Synthesis of Compound sub2-A-2

Under a nitrogen atmosphere, Compound 2-A (15 g, 58.3 mmol) and Compound 2-C (10 g, 64.2 mmol) were added to 300 ml of tetrahydrofuran (THF), and the mixture was stirred and refluxed. Afterwards, potassium carbonate (16.1 g, 116.7 mmol) was dissolved in 48 mL of water, and then added thereto. Thereafter, it was stirred sufficiently, followed by adding Tetrakis(triphenylphosphine) palladium (0) (1.3 g, 1.2 mmol). After a reaction time of 10 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-A-2 of 10.6 g. (Yield 63%, MS: [M+H]⁺=289).

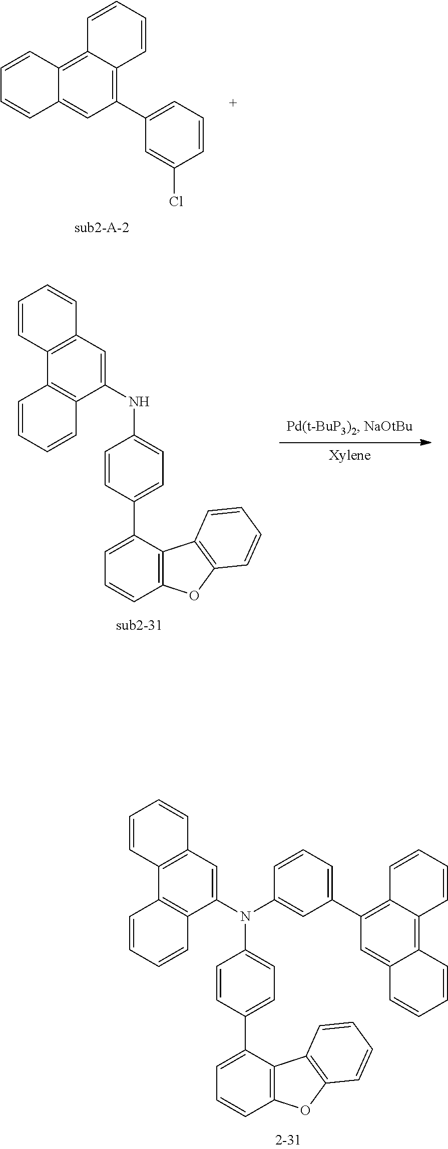 [SMILES: Clc1cccc(-c2cc3ccccc3c3ccccc23)c1 ; c1ccc2c(c1)cc(Nc1ccc(-c3cccc4oc5ccccc5c34)cc1)c1ccccc12 ; "Xylene" ; c1cc(-c2cc3ccccc3c3ccccc23)cc(N(c2ccc(-c3cccc4oc5ccccc5c34)cc2)c2cc3ccccc3c3ccccc23)c1]

### Step 2) Synthesis of Compound 2-31

Under a nitrogen atmosphere, Compound sub2-A-2 (10 g, 34.6 mmol) prepared in the above Step 1), Compound sub2-31 (15.1 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-31 of 16.7 g. (Yield 70%, MS: [M+H]⁺=688).

### Synthesis Example 2-32

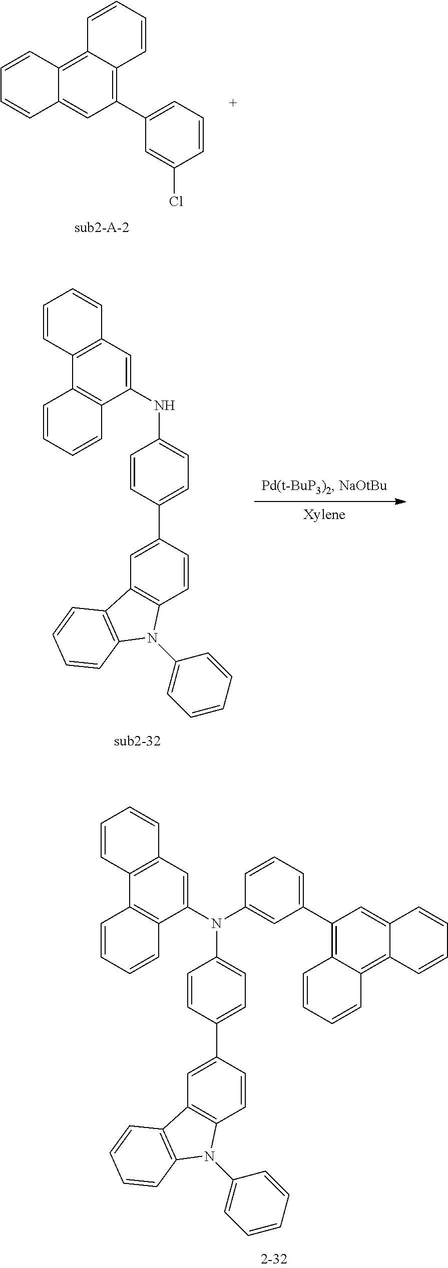 [SMILES: Clc1cccc(-c2cc3ccccc3c3ccccc23)c1 ; c1ccc(-n2c3ccccc3c3cc(-c4ccc(Nc5cc6ccccc6c6ccccc56)cc4)ccc32)cc1 ; "Xylene" ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1]

Under a nitrogen atmosphere, Compound sub2-A-2 (10 g, 34.6 mmol), Compound sub2-32 (17.7 g, 34.6 mmol), and sodium tert-butoxide (4.3 g, 45 mmol) were added to 200 ml of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-32 of 16.6 g. (Yield 63%, MS: [M+H]⁺=763).

### Synthesis Example 2-33

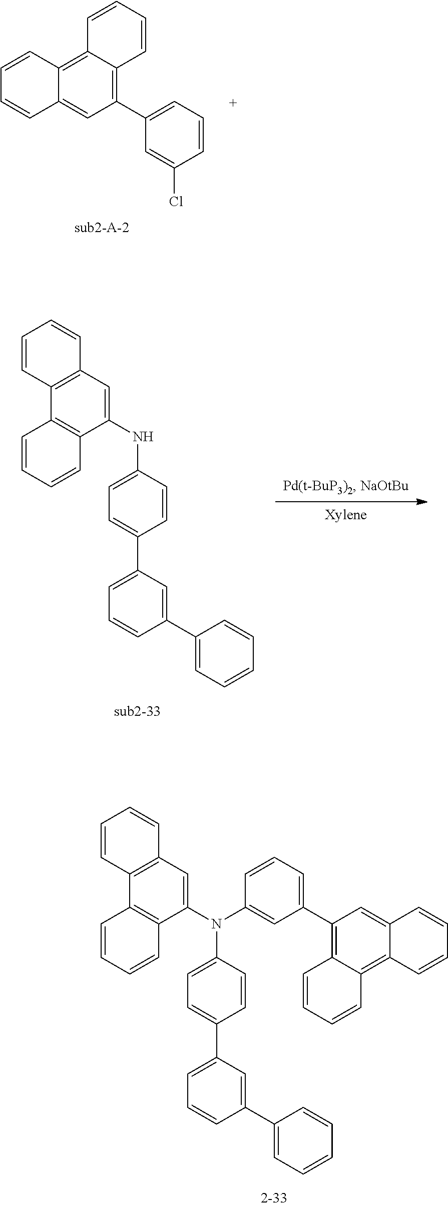 [SMILES: Clc1cccc(-c2cc3ccccc3c3ccccc23)c1 ; c1ccc(-c2cccc(-c3ccc(Nc4cc5ccccc5c5ccccc45)cc3)c2)cc1 ; "Xylene" ; c1ccc(-c2cccc(-c3ccc(N(c4cccc(-c5cc6ccccc6c6ccccc56)c4)c4cc5ccccc5c5ccccc45)cc3)c2)cc1]

Under a nitrogen atmosphere, Compound sub2-A-2 (10 g, 34.6 mmol), Compound sub2-33 (14.6 g, 34.6 mmol), and sodium tert-butoxide (4.3 g, 45 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-33 of 12.6 g. (Yield 54%, MS: [M+H]⁺=674).

### Synthesis Example 2-34

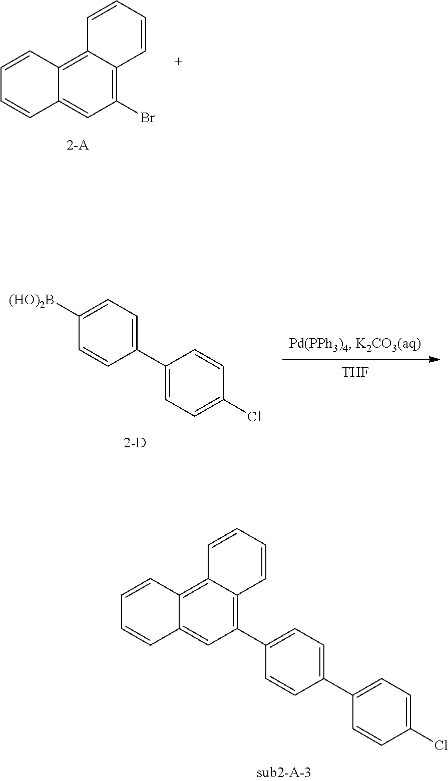 [SMILES: Brc1cc2ccccc2c2ccccc12 ; OB(O)c1ccc(-c2ccc(Cl)cc2)cc1 ; Clc1ccc(-c2ccc(-c3cc4ccccc4c4ccccc34)cc2)cc1 ; "THF"]

Step 1) Synthesis of Compound sub2-A-3

Under a nitrogen atmosphere, Compound 2-A (15 g, 58.3 mmol) and Compound 2-D (14.9 g, 64.2 mmol) were added to 300 mL of tetrahydrofuran (THF), and the mixture was stirred and refluxed. Afterwards, potassium carbonate (16.1 g, 116.7 mmol) was dissolved in 48 ml of water, and then added thereto. Thereafter, it was stirred sufficiently, followed by adding Tetrakis(triphenylphosphine) palladium (0) (1.3 g, 1.2 mmol). After a reaction time of 10 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-A-3 of 16.8 g. (Yield 79%, MS: [M+H]⁺=365).

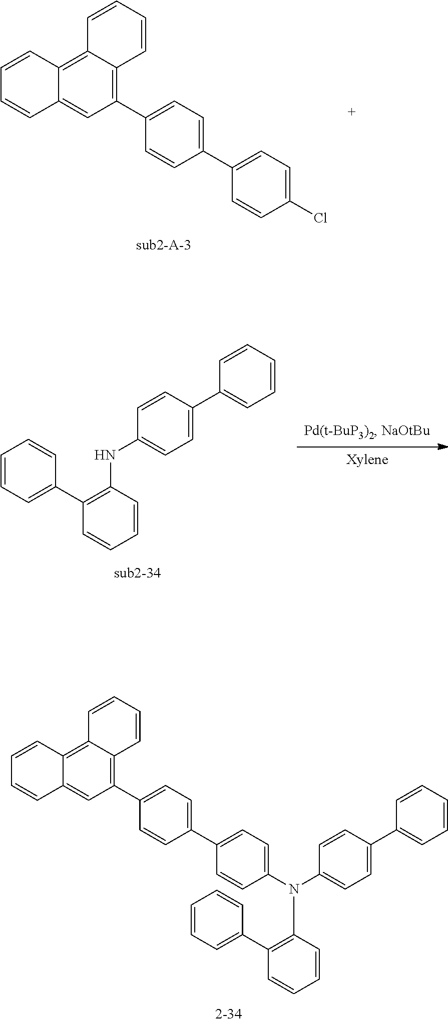 [SMILES: Clc1ccc(-c2ccc(-c3cc4ccccc4c4ccccc34)cc2)cc1 ; c1ccc(-c2ccc(Nc3ccccc3-c3ccccc3)cc2)cc1 ; "Xylene" ; c1ccc(-c2ccc(N(c3ccc(-c4ccc(-c5cc6ccccc6c6ccccc56)cc4)cc3)c3ccccc3-c3ccccc3)cc2)cc1]

### Step 2) Synthesis of Compound 2-34

Under a nitrogen atmosphere, Compound sub2-A-3 (10 g, 27.4 mmol) prepared in the above Step 1), Compound sub2-34 (8.8 g, 27.4 mmol), and sodium tert-butoxide (3.4 g, 35.7 mmol) were added to 200 ml of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-34 of 11.2 g. (Yield 63%, MS: [M+H]⁺=650).

### Synthesis Example 2-35

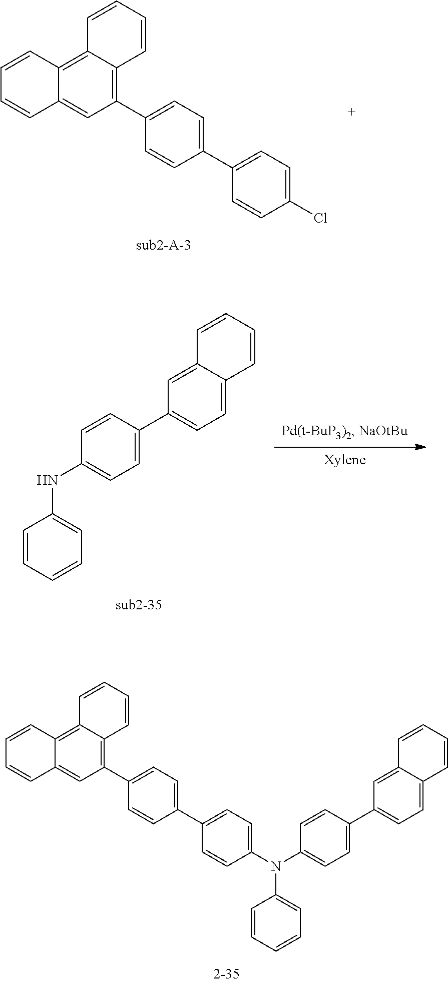 [SMILES: Clc1ccc(-c2ccc(-c3cc4ccccc4c4ccccc34)cc2)cc1 ; c1ccc(Nc2ccc(-c3ccc4ccccc4c3)cc2)cc1 ; "Xylene" ; c1ccc(N(c2ccc(-c3ccc(-c4cc5ccccc5c5ccccc45)cc3)cc2)c2ccc(-c3ccc4ccccc4c3)cc2)cc1]

Under a nitrogen atmosphere, Compound sub2-A-3 (10 g, 27.4 mmol), Compound sub2-35 (8.1 g, 27.4 mmol), and sodium tert-butoxide (3.4 g, 35.7 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-35 of 8.7 g. (Yield 63%, MS: [M+H]⁺=624).

### Synthesis Example 2-36

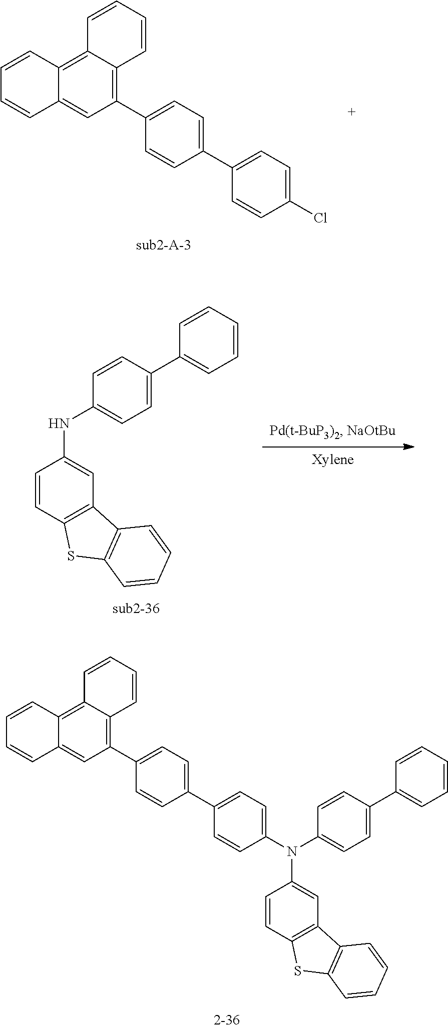 [SMILES: Clc1ccc(-c2ccc(-c3cc4ccccc4c4ccccc34)cc2)cc1 ; c1ccc(-c2ccc(Nc3ccc4sc5ccccc5c4c3)cc2)cc1 ; "Xylene" ; c1ccc(-c2ccc(N(c3ccc(-c4ccc(-c5cc6ccccc6c6ccccc56)cc4)cc3)c3ccc4sc5ccccc5c4c3)cc2)cc1]

Under a nitrogen atmosphere, Compound sub2-A-3 (10 g, 27.4 mmol), Compound sub2-36 (9.6 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-36 of 12.1 g. (Yield 65%, MS: [M+H]⁺=680).

### Synthesis Example 2-37

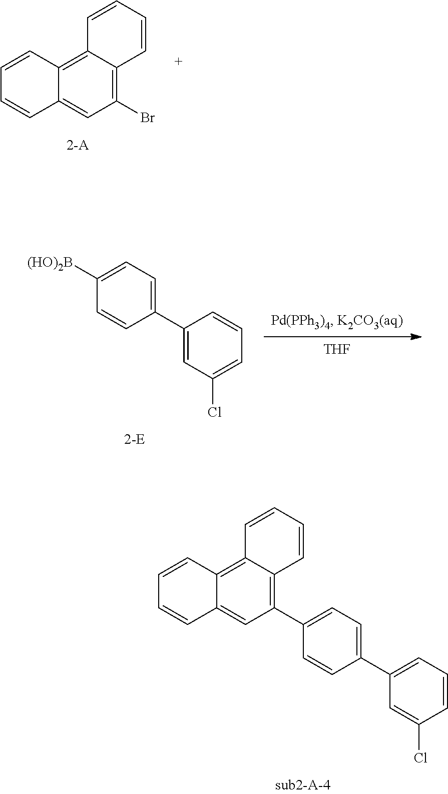 [SMILES: Brc1cc2ccccc2c2ccccc12 ; OB(O)c1ccc(-c2cccc(Cl)c2)cc1 ; Clc1cccc(-c2ccc(-c3cc4ccccc4c4ccccc34)cc2)c1 ; "THF"]

Step 1) Synthesis of Compound sub2-A-4

Under a nitrogen atmosphere, Compound 2-A (15 g, 58.3 mmol) and Compound 2-E (14.9 g, 64.2 mmol) were added to 300 mL of tetrahydrofuran (THF), and the mixture was stirred and refluxed. Afterwards, potassium carbonate (16.1 g, 116.7 mmol) was dissolved in 48 mL of water, and then added thereto. Thereafter, it was stirred sufficiently, followed by adding Tetrakis(triphenylphosphine) palladium (0) (1.3 g, 1.2 mmol). After a reaction time of 11 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-A-4 of 14.2 g. (Yield 67%, MS: [M+H]⁺=365).

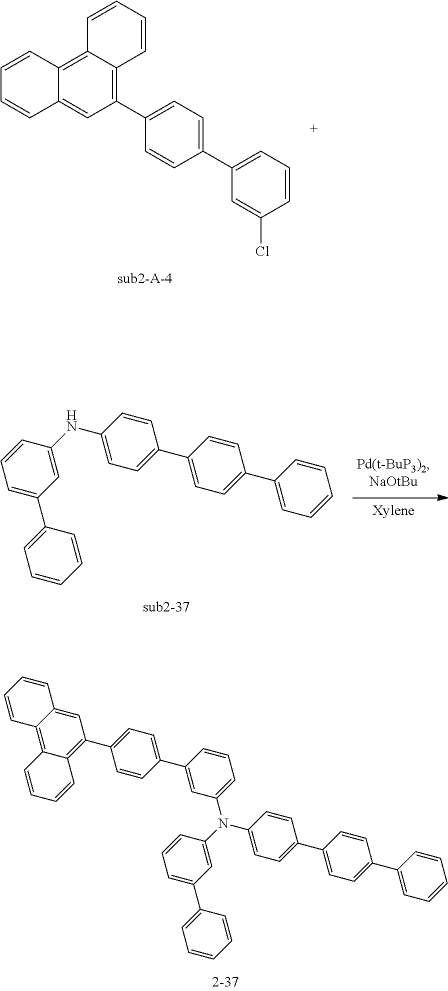 [SMILES: Clc1cccc(-c2ccc(-c3cc4ccccc4c4ccccc34)cc2)c1 ; c1ccc(-c2ccc(-c3ccc(Nc4cccc(-c5ccccc5)c4)cc3)cc2)cc1 ; "Xylene" ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1]

### Step 2) Synthesis of Compound 2-37

Under a nitrogen atmosphere, Compound sub2-A-4 (10 g, 27.4 mmol) prepared in the above Step 1), Compound sub2-37 (10.9 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-37 of 13.9 g. (Yield 70%, MS: [M+H]⁺=726).

### Synthesis Example 2-38

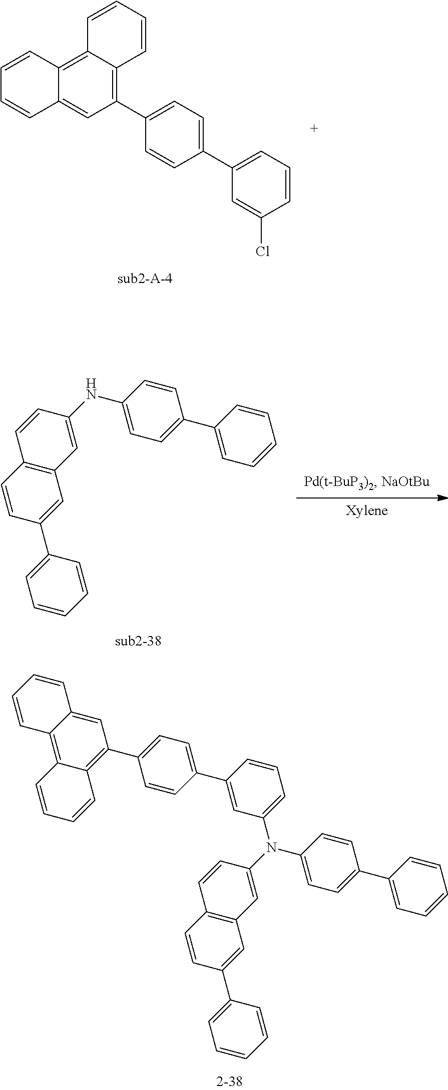 [SMILES: Clc1cccc(-c2ccc(-c3cc4ccccc4c4ccccc34)cc2)c1 ; c1ccc(-c2ccc(Nc3ccc4ccc(-c5ccccc5)cc4c3)cc2)cc1 ; "Xylene" ; c1ccc(-c2ccc(N(c3cccc(-c4ccc(-c5cc6ccccc6c6ccccc56)cc4)c3)c3ccc4ccc(-c5ccccc5)cc4c3)cc2)cc1]

Under a nitrogen atmosphere, Compound sub2-A-4 (10 g, 27.4 mmol), Compound sub2-38 (10.2 g, 27.4 mmol), and sodium tert-butoxide (3.4 g, 35.6 mmol) were added to 200 ml of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-38 of 10.7 g. (Yield 56%, MS: [M+H]⁺=700).

### Synthesis Example 2-39

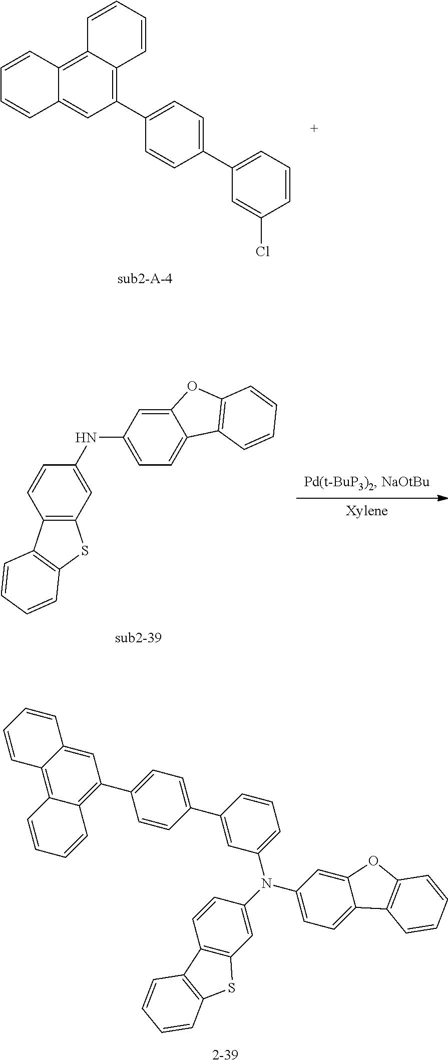 [SMILES: Clc1cccc(-c2ccc(-c3cc4ccccc4c4ccccc34)cc2)c1 ; c1ccc2c(c1)oc1cc(Nc3ccc4c(c3)sc3ccccc34)ccc12 ; "Xylene" ; c1cc(-c2ccc(-c3cc4ccccc4c4ccccc34)cc2)cc(N(c2ccc3c(c2)oc2ccccc23)c2ccc3c(c2)sc2ccccc23)c1]

Under a nitrogen atmosphere, Compound sub2-A-4 (10 g, 27.4 mmol), Compound sub2-39 (10 g, 27.4 mmol), and sodium tert-butoxide (3.4 g, 35.6 mmol) were added to 200 ml of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-39 of 11.8 g. (Yield 62%, MS: [M+H]⁺=694).

### Synthesis Example 2-40

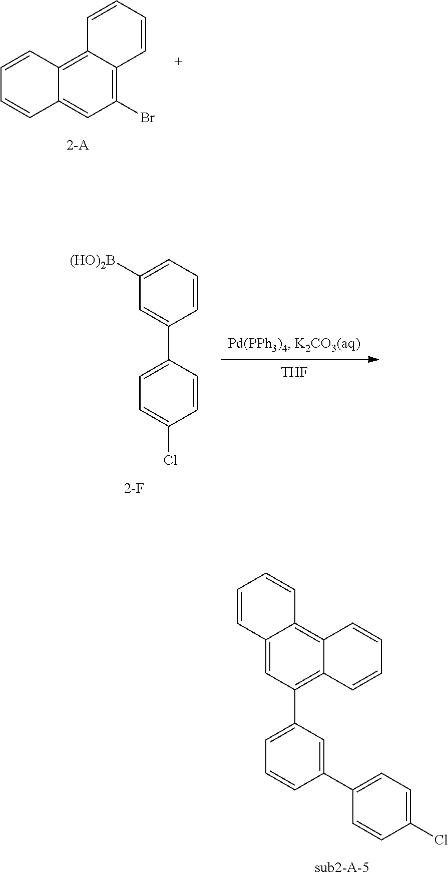 [SMILES: Brc1cc2ccccc2c2ccccc12 ; OB(O)c1cccc(-c2ccc(Cl)cc2)c1 ; "THF" ; Clc1ccc(-c2cccc(-c3cc4ccccc4c4ccccc34)c2)cc1]

Step 1) Synthesis of Compound sub2-A-5

Under a nitrogen atmosphere, Compound 2-A (15 g, 58.3 mmol) and Compound 2-F (14.9 g, 64.2 mmol) were added to 300 mL of tetrahydrofuran (THF), and the mixture was stirred and refluxed. Afterwards, potassium carbonate (16.1 g, 116.7 mmol) was dissolved in 48 mL of water, and then added thereto. Thereafter, it was stirred sufficiently, followed by adding Tetrakis(triphenylphosphine) palladium (0) (1.3 g, 1.2 mmol). After a reaction time of 8 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-A-5 of 14.4 g. (Yield 68%, MS: [M+H]⁺=365).

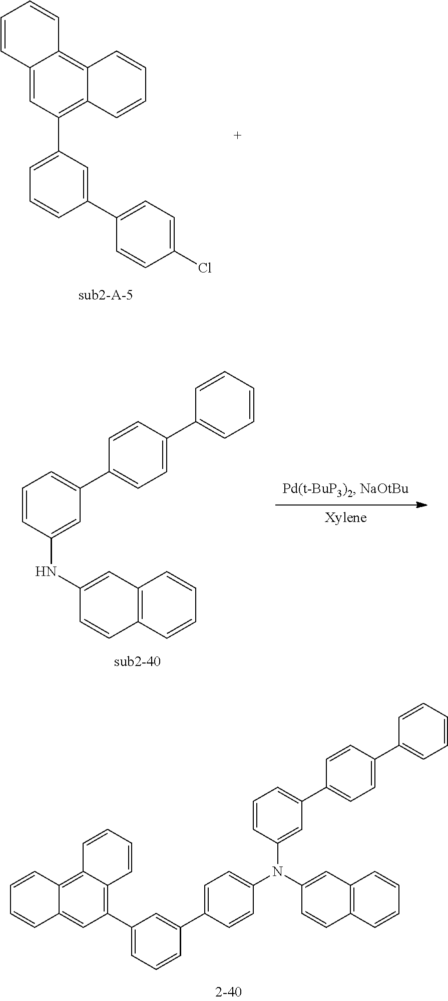 [SMILES: Clc1ccc(-c2cccc(-c3cc4ccccc4c4ccccc34)c2)cc1 ; c1ccc(-c2ccc(-c3cccc(Nc4ccc5ccccc5c4)c3)cc2)cc1 ; "Xylene" ; c1ccc(-c2ccc(-c3cccc(N(c4ccc(-c5cccc(-c6cc7ccccc7c7ccccc67)c5)cc4)c4ccc5ccccc5c4)c3)cc2)cc1]

### Step 2) Synthesis of Compound 2-40

Under a nitrogen atmosphere, Compound sub2-A-5 (10 g, 27.4 mmol) prepared in the above Step 1), Compound sub2-40 (10.2 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-40 of 10.7 g. (Yield 56%, MS: [M+H]⁺=700).

### Synthesis Example 2-41

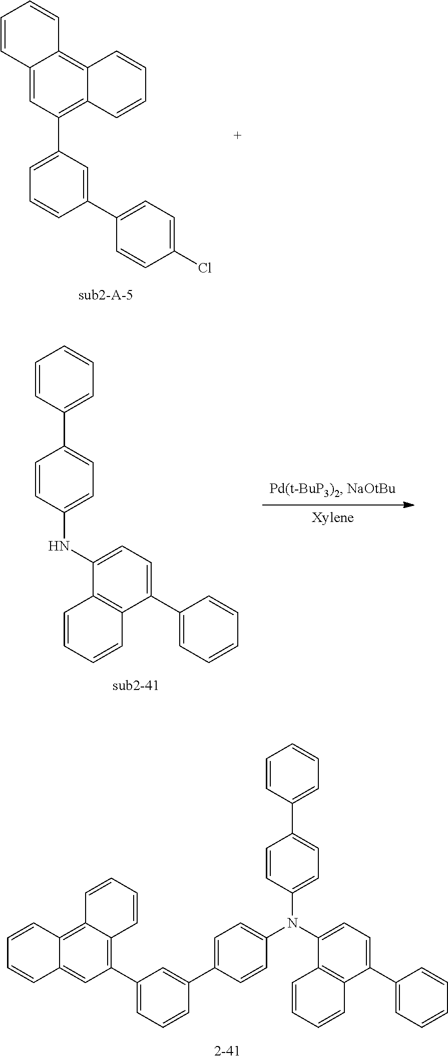 [SMILES: Clc1ccc(-c2cccc(-c3cc4ccccc4c4ccccc34)c2)cc1 ; c1ccc(-c2ccc(Nc3ccc(-c4ccccc4)c4ccccc34)cc2)cc1 ; "Xylene" ; c1ccc(-c2ccc(N(c3ccc(-c4cccc(-c5cc6ccccc6c6ccccc56)c4)cc3)c3ccc(-c4ccccc4)c4ccccc34)cc2)cc1]

Under a nitrogen atmosphere, Compound sub2-A-5 (10 g, 27.4 mmol), Compound sub2-41 (10.2 g, 27.4 mmol), and sodium tert-butoxide (3.4 g, 35.6 mmol) were added to 200 ml of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-41 of 9.8 g. (Yield 51%, MS: [M+H]⁺=700).

### Synthesis Example 2-42

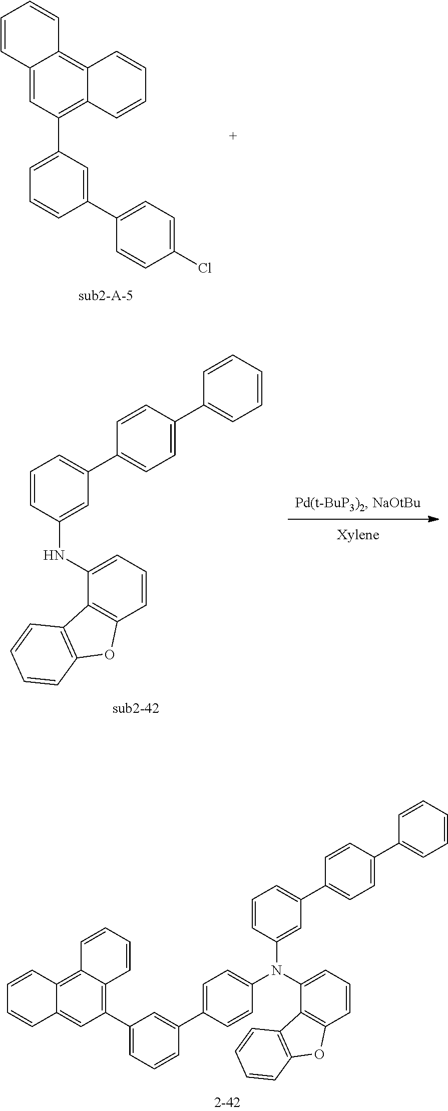 [SMILES: Clc1ccc(-c2cccc(-c3cc4ccccc4c4ccccc34)c2)cc1 ; c1ccc(-c2ccc(-c3cccc(Nc4cccc5oc6ccccc6c45)c3)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1]

Under a nitrogen atmosphere, Compound sub2-A-5 (10 g, 27.4 mmol), Compound sub2-42 (11.3 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-42 of 11.5 g. (Yield 57%, MS: [M+H]⁺=740).

### Synthesis Example 2-43

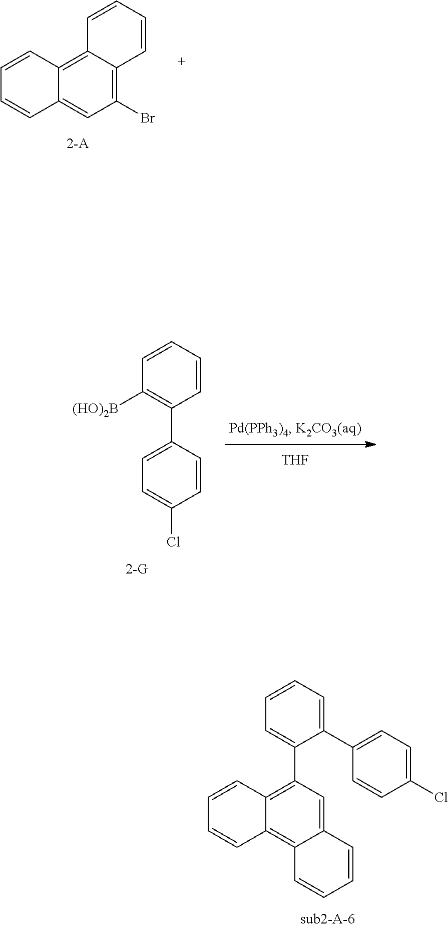 [SMILES: Brc1cc2ccccc2c2ccccc12 ; OB(O)c1ccccc1-c1ccc(Cl)cc1 ; "THF" ; Clc1ccc(-c2ccccc2-c2cc3ccccc3c3ccccc23)cc1]

Step 1) Synthesis of Compound sub2-A-6

Under a nitrogen atmosphere, Compound 2-A (15 g, 58.3 mmol) and Compound 2-G (14.9 g, 64.2 mmol) were added to 300 mL of tetrahydrofuran (THF), and the mixture was stirred and refluxed. Afterwards, potassium carbonate (16.1 g, 116.7 mmol) was dissolved in 48 ml of water, and then added thereto. Thereafter, it was stirred sufficiently, followed by adding Tetrakis(triphenylphosphine) palladium (0) (1.3 g, 1.2 mmol). After a reaction time of 9 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-A-6 of 14.7 g. (Yield 69%, MS: [M+H]⁺=365).

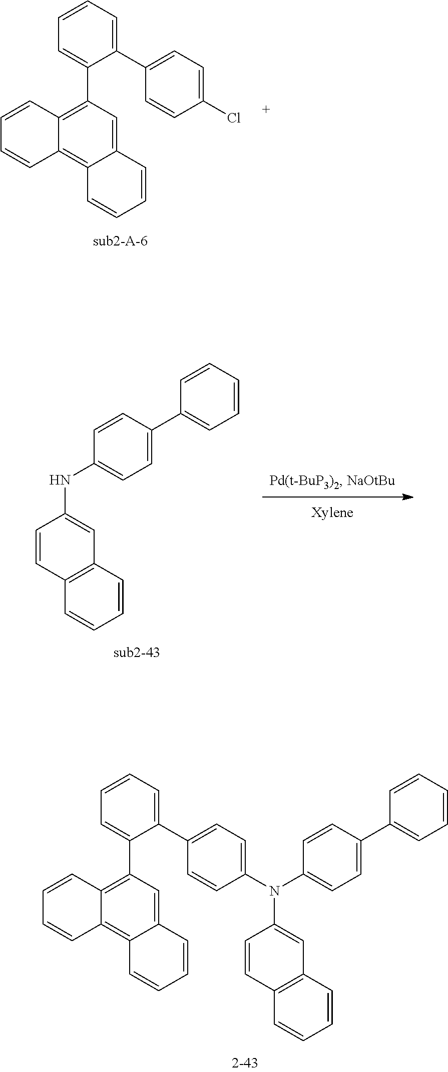 [SMILES: Clc1ccc(-c2ccccc2-c2cc3ccccc3c3ccccc23)cc1 ; c1ccc(-c2ccc(Nc3ccc4ccccc4c3)cc2)cc1 ; c1ccc(-c2ccc(N(c3ccc(-c4ccccc4-c4cc5ccccc5c5ccccc45)cc3)c3ccc4ccccc4c3)cc2)cc1]

### Step 2) Synthesis of Compound 2-43

Under a nitrogen atmosphere, Compound sub2-A-6 (10 g, 27.4 mmol) prepared in the above Step 1), Compound sub2-43 (8.1 g, 27.4 mmol), and sodium tert-butoxide (3.4 g, 35.6 mmol) were added to 200 ml of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-43 of 9.7 g. (Yield 57%, MS: [M+H]⁺=624).

### Synthesis Example 2-44

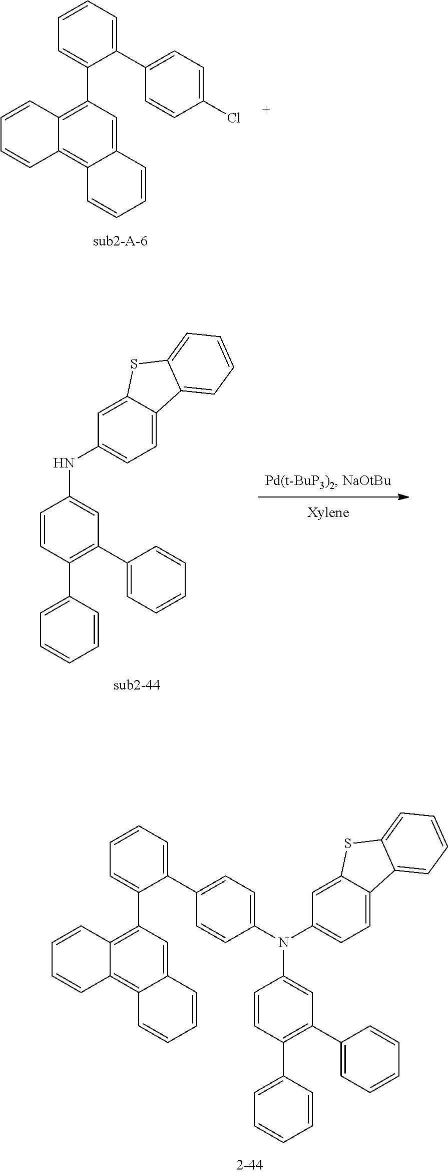 [SMILES: Clc1ccc(-c2ccccc2-c2cc3ccccc3c3ccccc23)cc1 ; c1ccc(-c2ccc(Nc3ccc4c(c3)sc3ccccc34)cc2-c2ccccc2)cc1 ; c1ccc(-c2ccc(N(c3ccc(-c4ccccc4-c4cc5ccccc5c5ccccc45)cc3)c3ccc4c(c3)sc3ccccc34)cc2-c2ccccc2)cc1]

Under a nitrogen atmosphere, Compound sub2-A-4 (10 g, 27.4 mmol), Compound sub2-44 (11.7 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-44 of 12 g. (Yield 58%, MS: [M+H]⁺=756).

### Synthesis Example 2-45

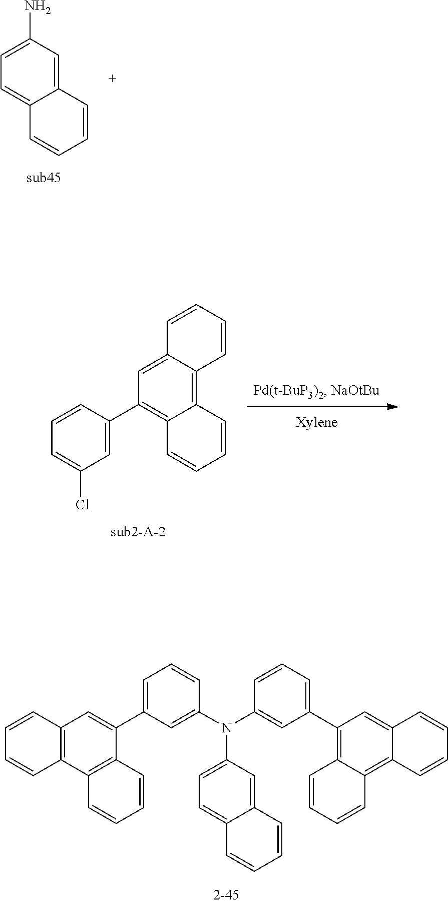 [SMILES: Nc1ccc2ccccc2c1 ; Clc1cccc(-c2cc3ccccc3c3ccccc23)c1 ; c1cc(-c2cc3ccccc3c3ccccc23)cc(N(c2cccc(-c3cc4ccccc4c4ccccc34)c2)c2ccc3ccccc3c2)c1]

Under a nitrogen atmosphere, Compound sub45 (10 g, 70.3 mmol), Compound sub2-A-2 (42.6 g, 147.7 mmol), and sodium tert-butoxide (16.9 g, 175.8 mmol) were added to 200 ml of xylene, and the mixture was stirred and refluxed. Thereafter, bis(tri-tert-butylphosphine) palladium (0) (0.7 g, 1.4 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-45 of 31 g. (Yield 68%, MS: [M+H]⁺=648).

### Synthesis Example 2-46

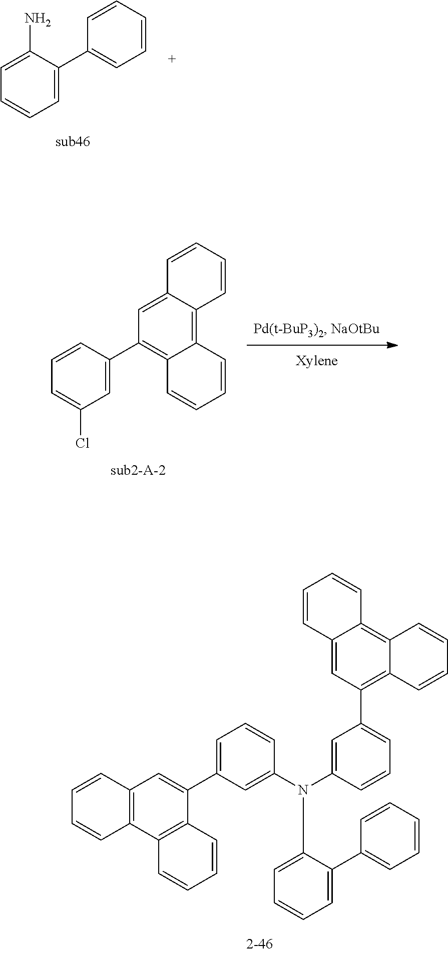 [SMILES: Nc1ccccc1-c1ccccc1 ; Clc1cccc(-c2cc3ccccc3c3ccccc23)c1 ; c1ccc(-c2ccccc2N(c2cccc(-c3cc4ccccc4c4ccccc34)c2)c2cccc(-c3cc4ccccc4c4ccccc34)c2)cc1]

Under a nitrogen atmosphere, Compound sub46 (10 g, 59.1 mmol), Compound sub2-A-2 (35.8 g, 124.1 mmol), and sodium tert-butoxide (14.2 g, 147.7 mmol) were added to 200 ml of xylene, and the mixture was stirred and refluxed. Thereafter, bis(tri-tert-butylphosphine) palladium (0) (0.6 g, 1.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-46 of 26.7 g. (Yield 67%, MS: [M+H]⁺=674).

### Synthesis Example 2-47

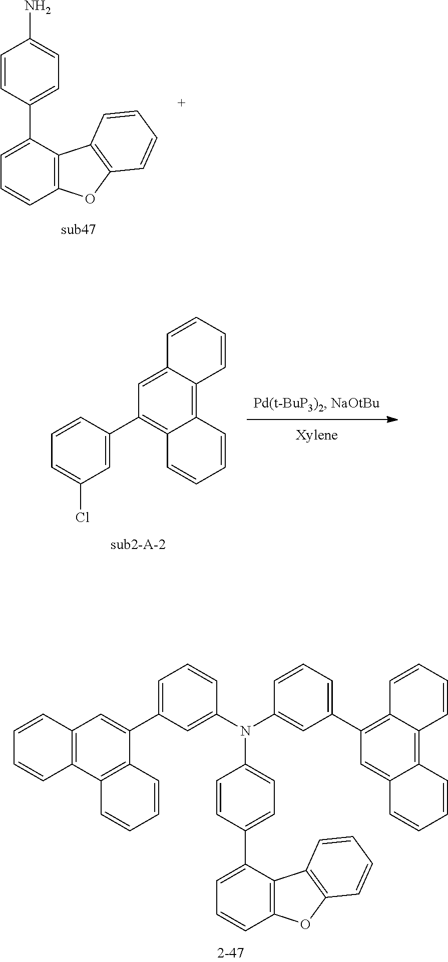 [SMILES: Nc1ccc(-c2cccc3oc4ccccc4c23)cc1 ; Clc1cccc(-c2cc3ccccc3c3ccccc23)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1]

Under a nitrogen atmosphere, Compound sub47 (10 g, 38.6 mmol), Compound sub2-A-2 (23.4 g, 81 mmol), and sodium tert-butoxide (9.3 g, 96.4 mmol) were added to 200 mL of xylene, and the mixture was stirred and refluxed. Thereafter, bis(tri-tert-butylphosphine) palladium (0) (0.4 g, 0.8 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-47 of 15 g. (Yield 51%, MS: [M+H]⁺=764).

### Synthesis Example 2-48

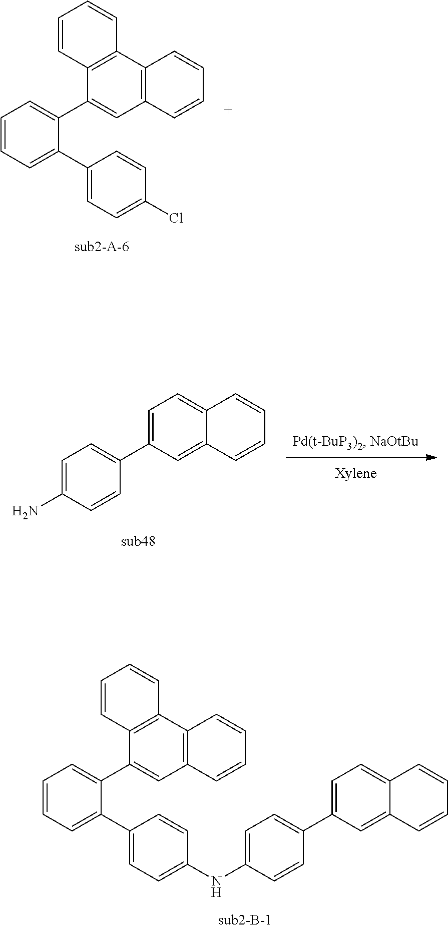 [SMILES: Clc1ccc(-c2ccccc2-c2cc3ccccc3c3ccccc23)cc1 ; Nc1ccc(-c2ccc3ccccc3c2)cc1 ; c1ccc(-c2cc3ccccc3c3ccccc23)c(-c2ccc(Nc3ccc(-c4ccc5ccccc5c4)cc3)cc2)c1]

Step 1) Synthesis of Compound sub2-B-1

Under a nitrogen atmosphere, Compound sub2-A-6 (10 g, 27.4 mmol), Compound sub48 (6 g, 27.4 mmol), and sodium tert-butoxide (2.9 g, 30.1 mmol) were added to 200 ml of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-1 of 9 g. (Yield 60%, MS: [M+H]⁺=548).

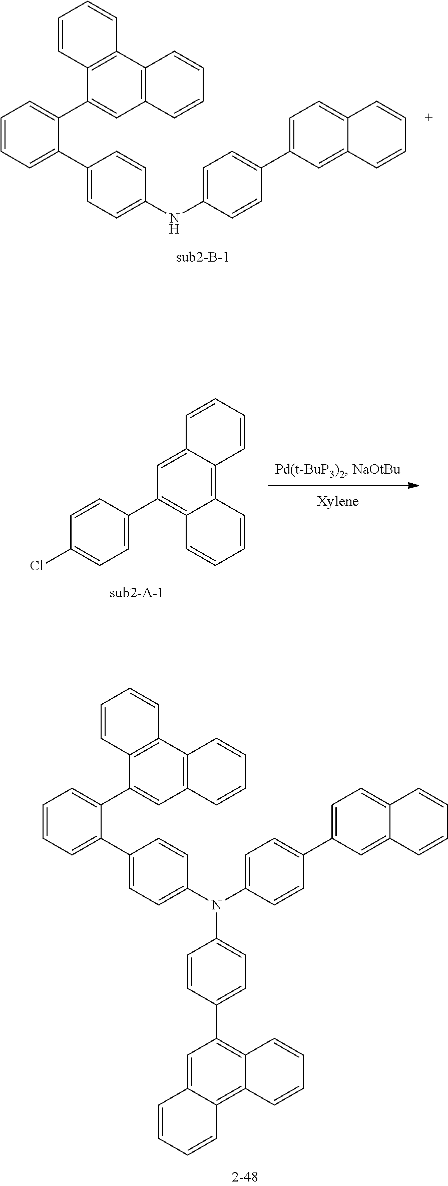 [SMILES: c1ccc(-c2cc3ccccc3c3ccccc23)c(-c2ccc(Nc3ccc(-c4ccc5ccccc5c4)cc3)cc2)c1 ; Clc1ccc(-c2cc3ccccc3c3ccccc23)cc1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1]

### Step 2) Synthesis of Compound 2-48

Under a nitrogen atmosphere, Compound sub2-B-1 (10 g, 18.3 mmol) prepared in the above Step 1), Compound sub2-A-1 (5.3 g, 18.3 mmol), and sodium tert-butoxide (2.3 g, 23.7 mmol) were added to 200 mL of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-48 of 7.7 g. (Yield 53%, MS: [M+H]⁺=800).

### Synthesis Example 2-49

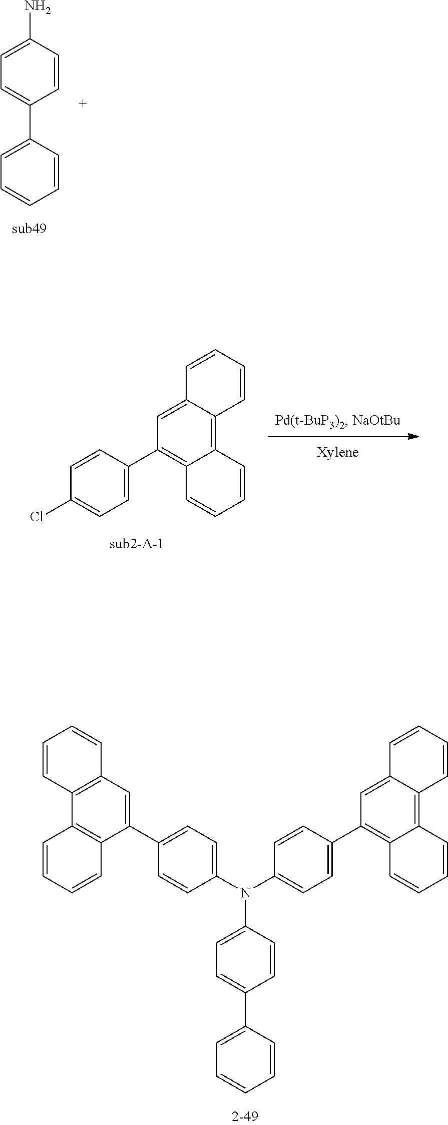 [SMILES: Nc1ccc(-c2ccccc2)cc1 ; Clc1ccc(-c2cc3ccccc3c3ccccc23)cc1 ; c1ccc(-c2ccc(N(c3ccc(-c4cc5ccccc5c5ccccc45)cc3)c3ccc(-c4cc5ccccc5c5ccccc45)cc3)cc2)cc1]

Under a nitrogen atmosphere, Compound sub49 (10 g, 59.1 mmol), Compound sub2-A-1 (35.8 g, 124.1 mmol), and sodium tert-butoxide (14.2 g, 147.7 mmol) were added to 200 mL of xylene, and the mixture was stirred and refluxed. Thereafter, bis(tri-tert-butylphosphine) palladium (0) (0.6 g, 1.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-49 of 22.7 g. (Yield 57%, MS: [M+H]⁺=674).

### Synthesis Example 2-50

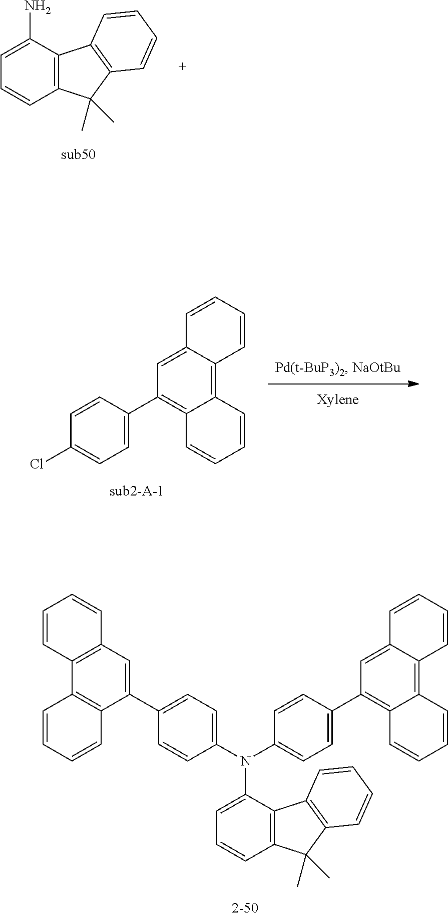 [SMILES: CC1(C)c2ccccc2-c2c(N)cccc21 ; Clc1ccc(-c2cc3ccccc3c3ccccc23)cc1 ; CC1(C)c2ccccc2-c2c(N(c3ccc(-c4cc5ccccc5c5ccccc45)cc3)c3ccc(-c4cc5ccccc5c5ccccc45)cc3)cccc21]

Under a nitrogen atmosphere, Compound sub50 (10 g, 47.8 mmol), Compound sub2-A-1 (29 g, 100.3 mmol), and sodium tert-butoxide (11.5 g, 119.5 mmol) were added to 200 ml of xylene, and the mixture was stirred and refluxed. Thereafter, bis(tri-tert-butylphosphine) palladium (0) (0.5 g, 1 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-50 of 23.9 g. (Yield 70%, MS: [M+H]⁺=714).

### Synthesis Example 2-51

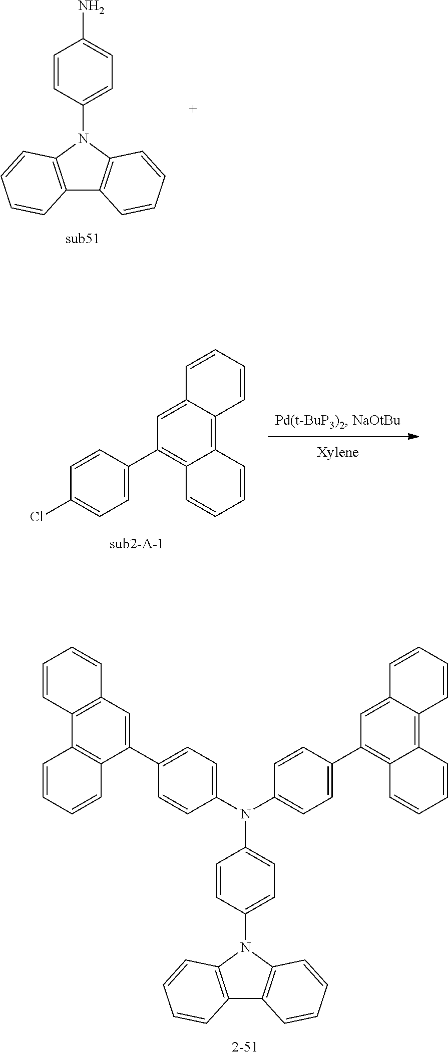 [SMILES: Nc1ccc(-n2c3ccccc3c3ccccc32)cc1 ; Clc1ccc(-c2cc3ccccc3c3ccccc23)cc1 ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12]

Under a nitrogen atmosphere, Compound sub51 (10 g, 38.7 mmol), Compound sub2-A-1 (23.5 g, 81.3 mmol), and sodium tert-butoxide (9.3 g, 96.8 mmol) were added to 200 mL of xylene, and the mixture was stirred and refluxed. Thereafter, bis(tri-tert-butylphosphine) palladium (0) (0.4 g, 0.8 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-51 of 16.8 g. (Yield 57%, MS: [M+H]⁺=763).

### Synthesis Example 2-52

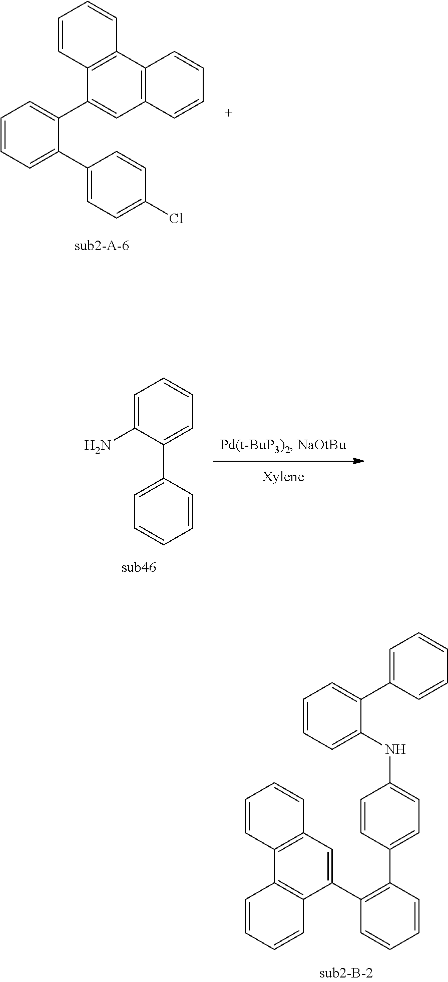 [SMILES: Clc1ccc(-c2ccccc2-c2cc3ccccc3c3ccccc23)cc1 ; Nc1ccccc1-c1ccccc1 ; c1ccc(-c2ccccc2Nc2ccc(-c3ccccc3-c3cc4ccccc4c4ccccc34)cc2)cc1]

Step 1) Synthesis of Compound sub2-B-2

Under a nitrogen atmosphere, Compound sub2-A-6 (10 g, 27.4 mmol), Compound sub46 (4.6 g, 27.4 mmol), and sodium tert-butoxide (2.9 g, 30.1 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-2 of 9.4 g. (Yield 69%, MS: [M+H]⁺=498).

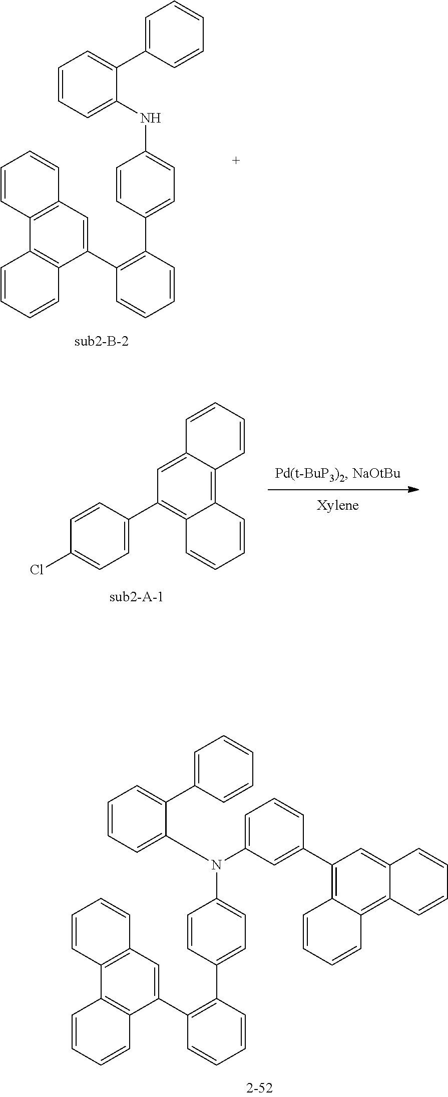 [SMILES: c1ccc(-c2ccccc2Nc2ccc(-c3ccccc3-c3cc4ccccc4c4ccccc34)cc2)cc1 ; Clc1ccc(-c2cc3ccccc3c3ccccc23)cc1 ; c1ccc(-c2ccccc2N(c2ccc(-c3ccccc3-c3cc4ccccc4c4ccccc34)cc2)c2cccc(-c3cc4ccccc4c4ccccc34)c2)cc1]

### Step 2) Synthesis of Compound 2-52

Under a nitrogen atmosphere, Compound sub2-B-2 (10 g, 20.1 mmol) prepared in the above Step 1), Compound sub2-A-1 (5.8 g, 20.1 mmol), and sodium tert-butoxide (2.5 g, 26.1 mmol) were added to 200 ml of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-52 of 8.3 g. (Yield 55%, MS: [M+H]⁺=750).

### Synthesis Example 2-53

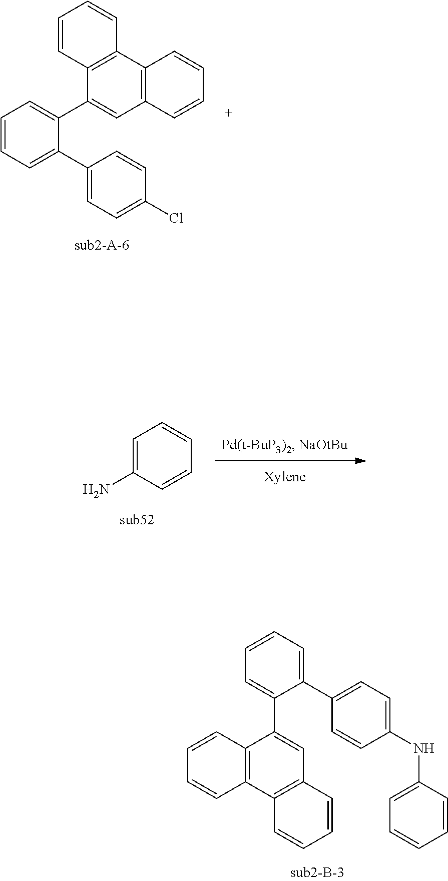 [SMILES: Clc1ccc(-c2ccccc2-c2cc3ccccc3c3ccccc23)cc1 ; Nc1ccccc1 ; c1ccc(Nc2ccc(-c3ccccc3-c3cc4ccccc4c4ccccc34)cc2)cc1]

Step 1) Synthesis of Compound sub2-B-3

Under a nitrogen atmosphere, Compound sub2-A-6 (10 g, 27.4 mmol), Compound sub52 (2.6 g, 27.4 mmol), and sodium tert-butoxide (2.9 g, 30.1 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-3 of 5.9 g. (Yield 51%, MS: [M+H]⁺=422).

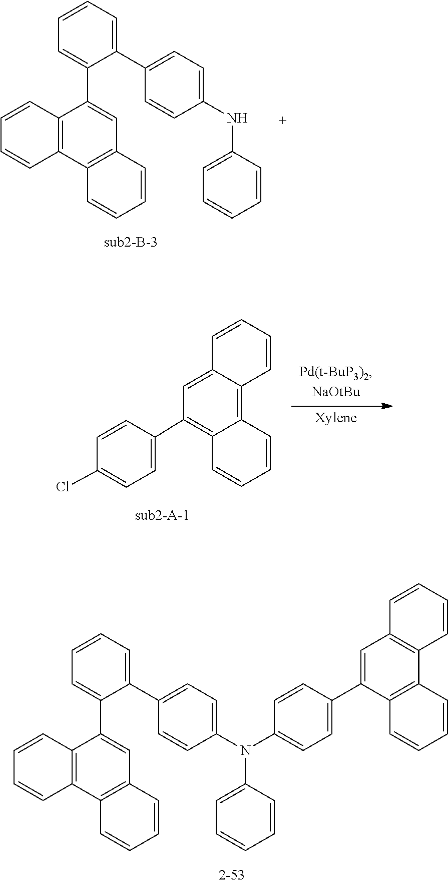 [SMILES: c1ccc(Nc2ccc(-c3ccccc3-c3cc4ccccc4c4ccccc34)cc2)cc1 ; Clc1ccc(-c2cc3ccccc3c3ccccc23)cc1 ; c1ccc(N(c2ccc(-c3ccccc3-c3cc4ccccc4c4ccccc34)cc2)c2ccc(-c3cc4ccccc4c4ccccc34)cc2)cc1]

### Step 2) Synthesis of Compound 2-53

Under a nitrogen atmosphere, Compound sub2-B-3 (10 g, 23.7 mmol) prepared in the above Step 1), Compound sub2-A-1 (6.9 g, 23.7 mmol), and sodium tert-butoxide (3 g, 30.8 mmol) were added to 200 mL of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-53 of 9.3 g. (Yield 58%, MS: [M+H]⁺=674).

### Synthesis Example 2-54

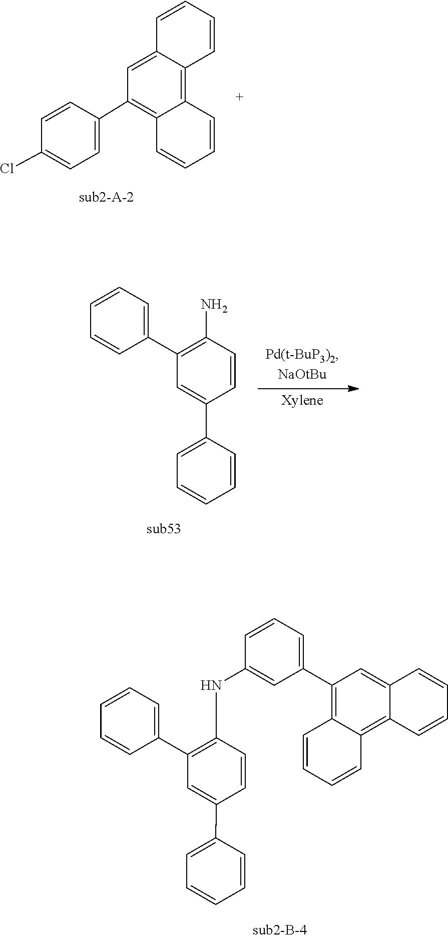 [SMILES: Clc1ccc(-c2cc3ccccc3c3ccccc23)cc1 ; Nc1ccc(-c2ccccc2)cc1-c1ccccc1 ; c1ccc(-c2ccc(Nc3cccc(-c4cc5ccccc5c5ccccc45)c3)c(-c3ccccc3)c2)cc1]

Step 1) Synthesis of Compound sub2-B-4

Under a nitrogen atmosphere, Compound sub2-A-2 (10 g, 34.6 mmol), Compound sub53 (8.5 g, 34.6 mmol), and sodium tert-butoxide (3.7 g, 38.1 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-4 of 11.5 g. (Yield 67%, MS: [M+H]⁺=498).

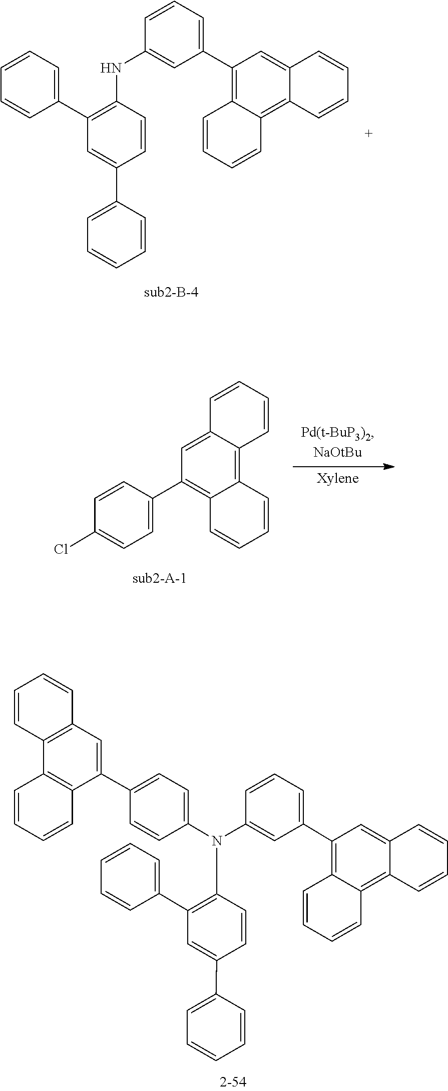 [SMILES: c1ccc(-c2ccc(Nc3cccc(-c4cc5ccccc5c5ccccc45)c3)c(-c3ccccc3)c2)cc1 ; Clc1ccc(-c2cc3ccccc3c3ccccc23)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1]

### Step 2) Synthesis of Compound 2-54

Under a nitrogen atmosphere, Compound sub2-B-4 (10 g, 20.1 mmol) prepared in the above Step 1), Compound sub2-A-1 (5.8 g, 20.1 mmol), and sodium tert-butoxide (2.5 g, 26.1 mmol) were added to 200 mL of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-54 of 7.5 g. (Yield 58%, MS: [M+H]⁺=750).

### Synthesis Example 2-55

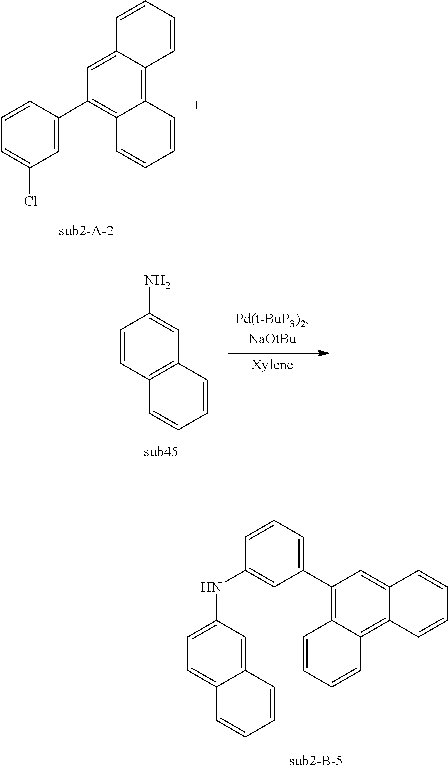 [SMILES: Clc1cccc(-c2cc3ccccc3c3ccccc23)c1 ; Nc1ccc2ccccc2c1 ; c1cc(Nc2ccc3ccccc3c2)cc(-c2cc3ccccc3c3ccccc23)c1]

Step 1) Synthesis of Compound sub2-B-5

Under a nitrogen atmosphere, Compound sub2-A-2 (10 g, 34.6 mmol), Compound sub45 (5 g, 34.6 mmol), and sodium tert-butoxide (3.7 g, 38.1 mmol) were added to 200 ml of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-5 of 9.3 g. (Yield 68%, MS: [M+H]⁺=396).

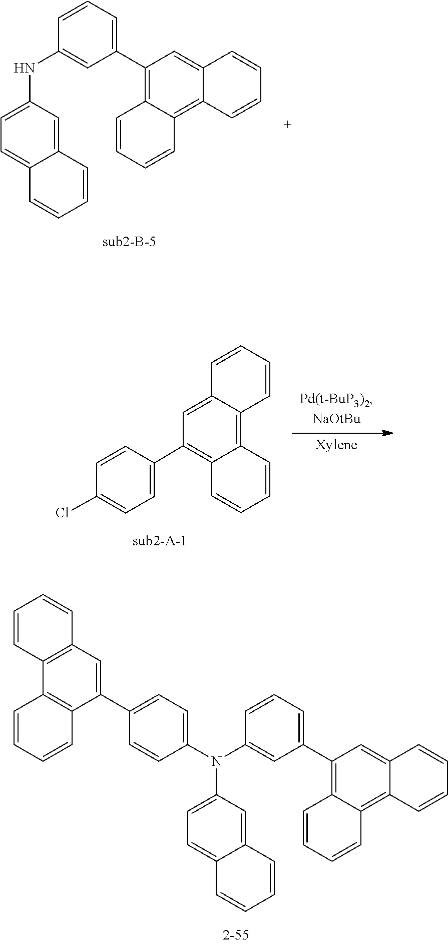 [SMILES: c1cc(Nc2ccc3ccccc3c2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc3ccccc3c2)c1]

### Step 2) Synthesis of Compound 2-55

Under a nitrogen atmosphere, Compound sub2-B-5 (10 g, 25.3 mmol) prepared in the above Step 1), Compound sub2-A-1 (7.3 g, 25.3 mmol), and sodium tert-butoxide (3.2 g, 32.9 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-55 of 10 g. (Yield 61%, MS: [M+H]⁺=648).

### Synthesis Example 2-56

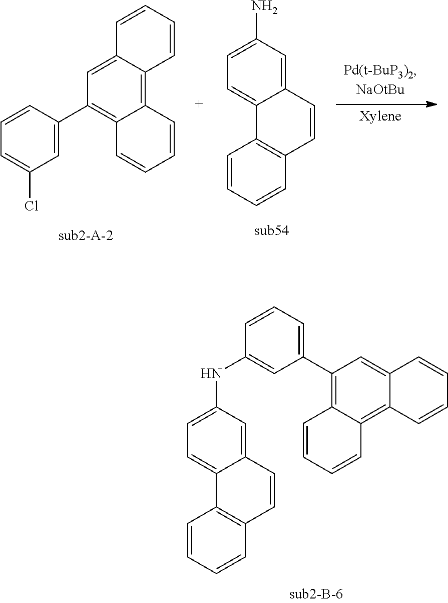 [SMILES: Clc1cccc(-c2cc3ccccc3c3ccccc23)c1 ; Nc1ccc2c(ccc3ccccc32)c1 ; c1cc(Nc2ccc3c(ccc4ccccc43)c2)cc(-c2cc3ccccc3c3ccccc23)c1]

Step 1) Synthesis of Compound sub2-B-6

Under a nitrogen atmosphere, Compound sub2-A-2 (10 g, 34.6 mmol), Compound sub54 (6.7 g, 34.6 mmol), and sodium tert-butoxide (3.7 g, 38.1 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-6 of 8.6 g. (Yield 56%, MS: [M+H]⁺=446).

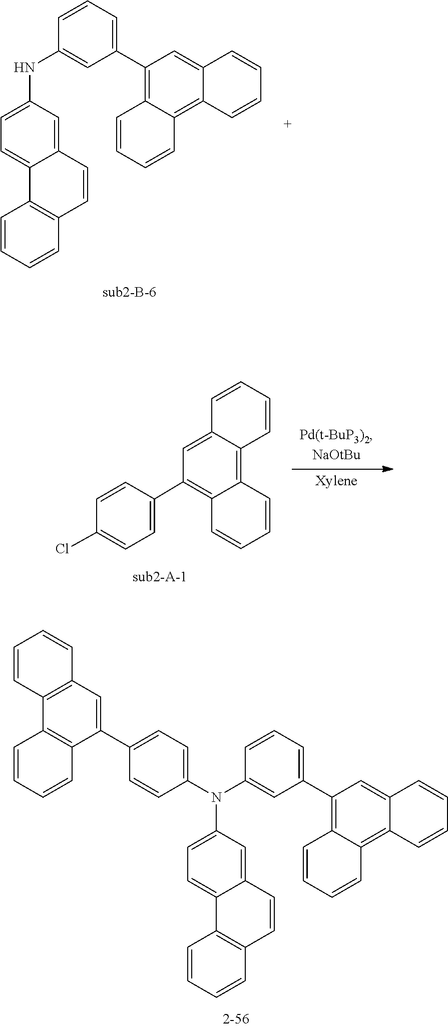 [SMILES: c1cc(Nc2ccc3c(ccc4ccccc43)c2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc3c(ccc4ccccc43)c2)c1]

### Step 2) Synthesis of Compound 2-56

Under a nitrogen atmosphere, Compound sub2-B-6 (10 g, 22.4 mmol) prepared in the above Step 1), Compound sub2-A-1 (6.5 g, 22.4 mmol), and sodium tert-butoxide (2.8 g, 29.2 mmol) were added to 200 ml of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-56 of 8.8 g. (Yield 56%, MS: [M+H]⁺=698).

### Synthesis Example 2-57

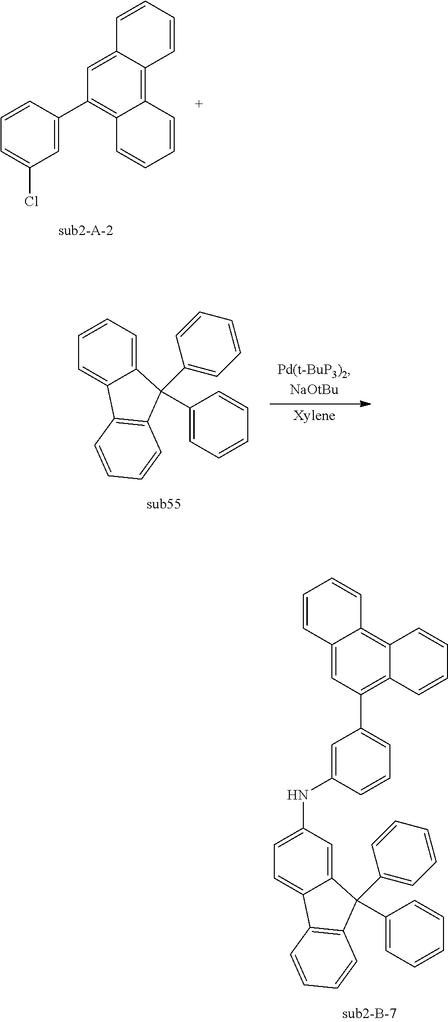

Step 1) Synthesis of Compound sub2-B-7

Under a nitrogen atmosphere, Compound sub2-A-2 (10 g, 34.6 mmol), Compound sub55 (11.5 g, 34.6 mmol), and sodium tert-butoxide (3.7 g, 38.1 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-7 of 13.2 g. (Yield 65%, MS: [M+H]⁺=586).

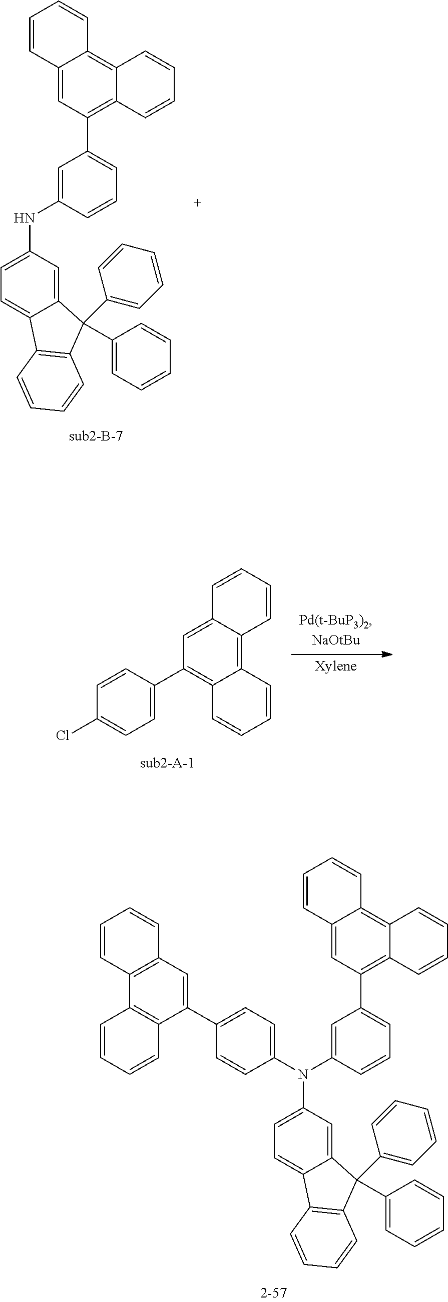 [SMILES: c1ccc(C2(c3ccccc3)c3ccccc3-c3ccc(Nc4cccc(-c5cc6ccccc6c6ccccc56)c4)cc32)cc1 ; Clc1ccc(-c2cc3ccccc3c3ccccc23)cc1 ; c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1]

### Step 2) Synthesis of Compound 2-57

Under a nitrogen atmosphere, Compound sub2-B-7 (10 g, 17.1 mmol) prepared in the above Step 1), Compound sub2-A-1 (4.9 g, 17.1 mmol), and sodium tert-butoxide (2.1 g, 22.2 mmol) were added to 200 mL of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-57 of 7.7 g. (Yield 54%, MS: [M+H]⁺=838).

### Synthesis Example 2-58

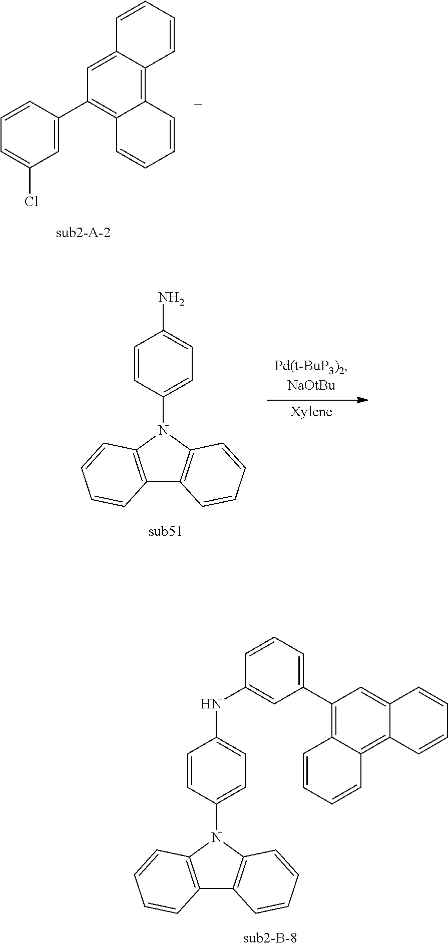 [SMILES: Clc1cccc(-c2cc3ccccc3c3ccccc23)c1 ; Nc1ccc(-n2c3ccccc3c3ccccc32)cc1 ; c1cc(Nc2ccc(-n3c4ccccc4c4ccccc43)cc2)cc(-c2cc3ccccc3c3ccccc23)c1]

Step 1) Synthesis of Compound sub2-B-8

Under a nitrogen atmosphere, Compound sub2-A-2 (10 g, 34.6 mmol), Compound sub51 (8.9 g, 34.6 mmol), and sodium tert-butoxide (3.7 g, 38.1 mmol) were added to 200 mL of xylene, and the mixture was stirred and refluxed. Thereafter, bis(tri-tert-butylphosphine) palladium (0) (0.2 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-8 of 10.8 g. (Yield 61%, MS: [M+H]⁺=511).

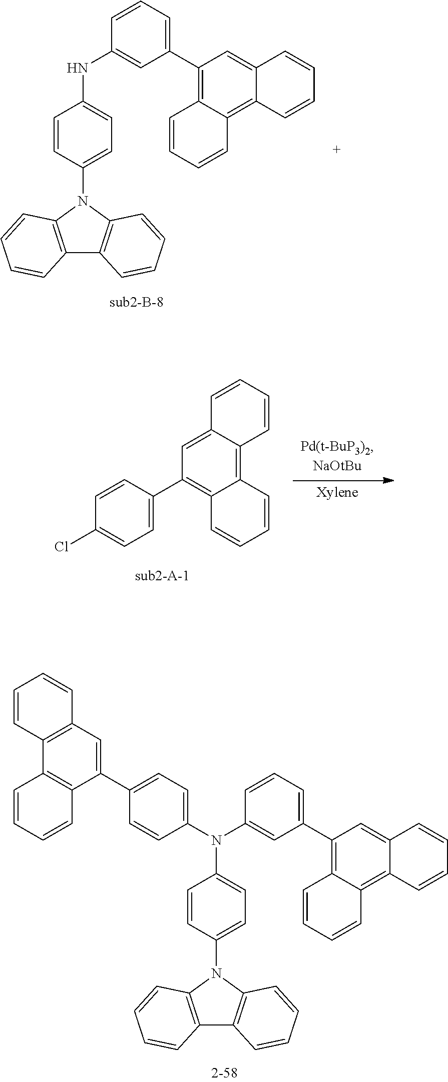 [SMILES: c1cc(Nc2ccc(-n3c4ccccc4c4ccccc43)cc2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1]

### Step 2) Synthesis of Compound 2-58

Under a nitrogen atmosphere, Compound sub2-B-8 (10 g, 19.6 mmol) prepared in the above Step 1), Compound sub2-A-1 (5.7 g, 19.6 mmol), sodium tert-butoxide (2.4 g, 25.5 mmol) were added to 200 ml of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-58 of 7.6 g. (Yield 51%, MS: [M+H]⁺=763).

### Synthesis Example 2-59

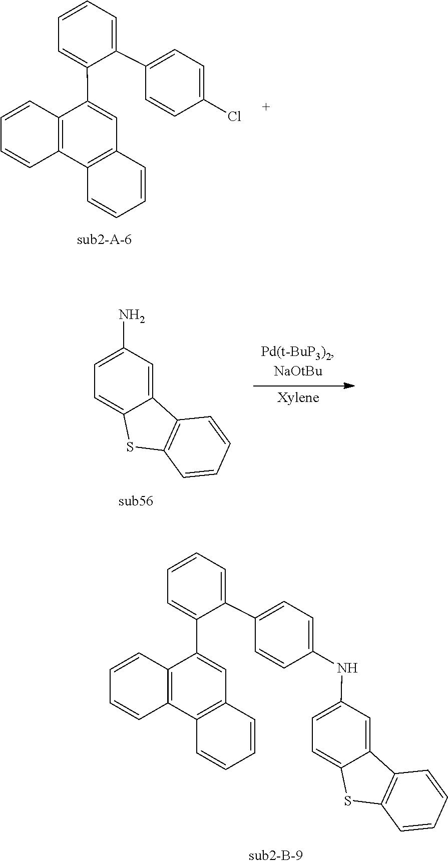 [SMILES: Clc1ccc(-c2ccccc2-c2cc3ccccc3c3ccccc23)cc1 ; Nc1ccc2sc3ccccc3c2c1 ; c1ccc(-c2cc3ccccc3c3ccccc23)c(-c2ccc(Nc3ccc4sc5ccccc5c4c3)cc2)c1]

Step 1) Synthesis of Compound sub2-B-9

Under a nitrogen atmosphere, Compound sub2-A-6 (10 g, 27.4 mmol), Compound sub56 (5.5 g, 27.4 mmol), and sodium tert-butoxide (2.9 g, 30.2 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-9 of 7.5 g. (Yield 52%, MS: [M+H]⁺=528).

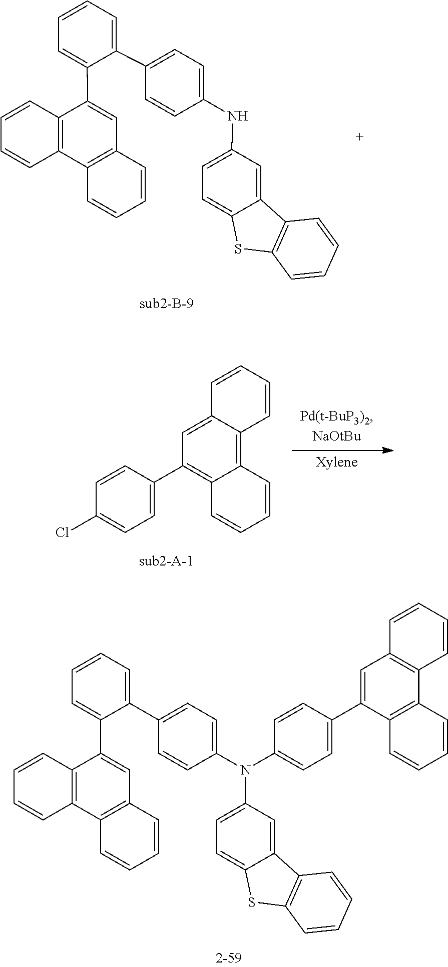 [SMILES: c1ccc(-c2cc3ccccc3c3ccccc23)c(-c2ccc(Nc3ccc4sc5ccccc5c4c3)cc2)c1 ; Clc1ccc(-c2cc3ccccc3c3ccccc23)cc1 ; c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1]

### Step 2) Synthesis of Compound 2-59

Under a nitrogen atmosphere, Compound sub2-B-9 (10 g, 19 mmol) prepared in the above Step 1), Compound sub2-A-1 (5.5 g, 19 mmol), and sodium tert-butoxide (2.4 g, 24.6 mmol) were added to 200 mL of xylene, and the mixture was stirred and refluxed. Thereafter, bis(tri-tert-butylphosphine) palladium (0) (0.1 g, 0.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-59 of 8.7 g. (Yield 59%, MS: [M+H]⁺=780).

### Synthesis Example 2-60

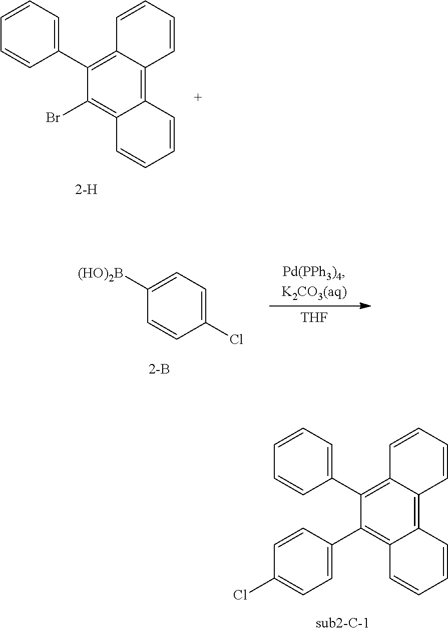 [SMILES: Brc1c(-c2ccccc2)c2ccccc2c2ccccc12 ; OB(O)c1ccc(Cl)cc1 ; Clc1ccc(-c2c(-c3ccccc3)c3ccccc3c3ccccc23)cc1 ; "THF"]

Step 1) Synthesis of Compound sub2-C-1

Under a nitrogen atmosphere, Compound 2-H (15 g, 45 mmol) and Compound 2-B (7.7 g, 49.5 mmol) were added to 300 mL of tetrahydrofuran (THF), and the mixture was stirred and refluxed. Afterwards, potassium carbonate (12.4 g, 90 mmol) was dissolved in 37 mL of water, and then added thereto. Thereafter, it was stirred sufficiently, followed by adding Tetrakis(triphenylphosphine) palladium (0) (1 g, 0.9 mmol). After a reaction time of 11 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-C-1 of 12.3 g. (Yield 75%, MS: [M+H]⁺=365).

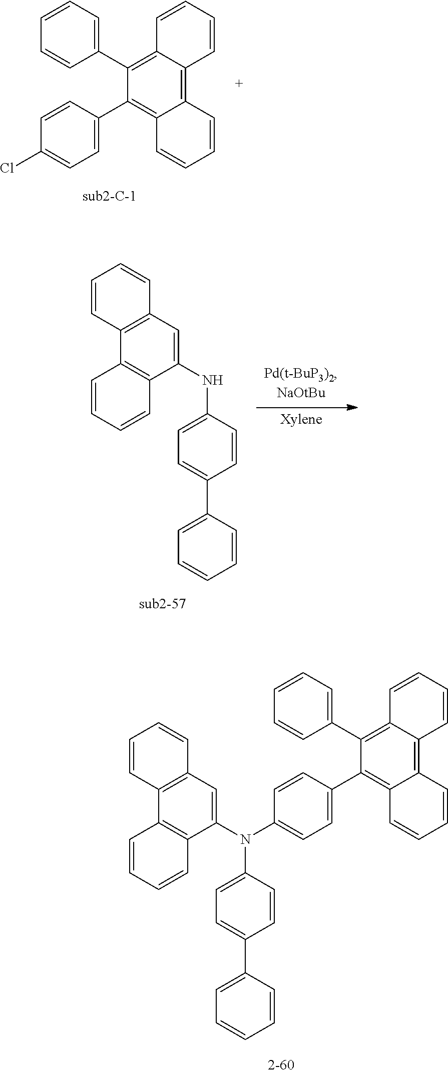 [SMILES: Clc1ccc(-c2c(-c3ccccc3)c3ccccc3c3ccccc23)cc1 ; c1ccc(-c2ccc(Nc3cc4ccccc4c4ccccc34)cc2)cc1 ; c1ccc(-c2ccc(N(c3ccc(-c4c(-c5ccccc5)c5ccccc5c5ccccc45)cc3)c3cc4ccccc4c4ccccc34)cc2)cc1]

### Step 2) Synthesis of Compound 2-60

Under a nitrogen atmosphere, Compound sub2-C-1 (10 g, 27.4 mmol) prepared in the above Step 1), Compound sub2-57 (9.5 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-60 of 12.7 g. (Yield 69%, MS: [M+H]⁺=674).

### Synthesis Example 2-61

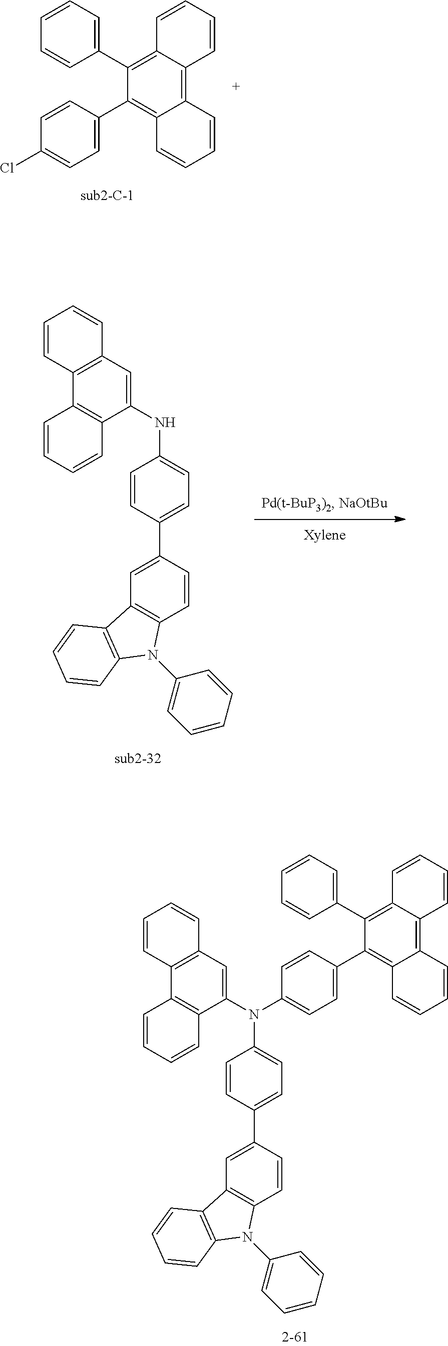 [SMILES: Clc1ccc(-c2c(-c3ccccc3)c3ccccc3c3ccccc23)cc1 ; c1ccc(-n2c3ccccc3c3cc(-c4ccc(Nc5cc6ccccc6c6ccccc56)cc4)ccc32)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1]

Under a nitrogen atmosphere, Compound sub2-C-1 (10 g, 27.4 mmol), Compound sub2-32 (14 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-61 of 12.6 g. (Yield 55%, MS: [M+H]⁺=839).

### Synthesis Example 2-62

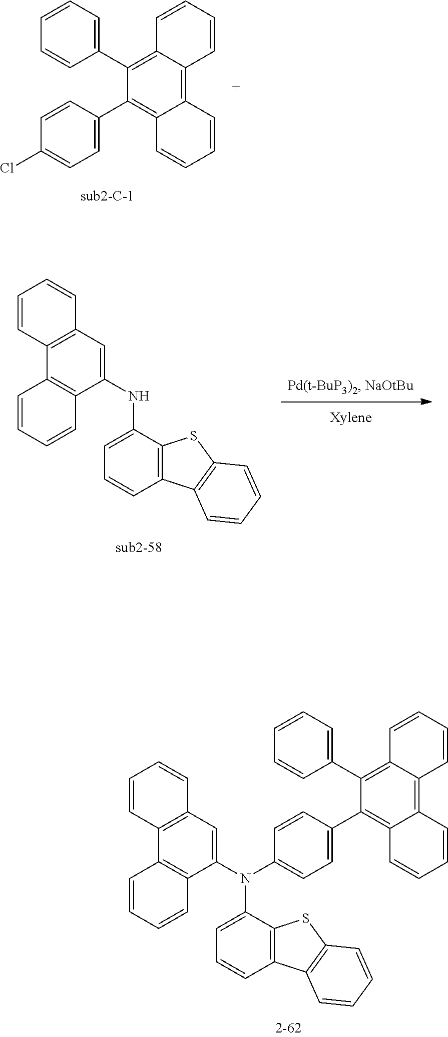 [SMILES: Clc1ccc(-c2c(-c3ccccc3)c3ccccc3c3ccccc23)cc1 ; c1ccc2c(c1)cc(Nc1cccc3c1sc1ccccc13)c1ccccc12 ; c1ccc(-c2c(-c3ccc(N(c4cc5ccccc5c5ccccc45)c4cccc5c4sc4ccccc45)cc3)c3ccccc3c3ccccc23)cc1]

Under a nitrogen atmosphere, Compound sub2-C-1 (10 g, 27.4 mmol), Compound sub2-58 (10.3 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-62 of 12.5 g. (Yield 65%, MS: [M+H]⁺=704).

### Synthesis Example 2-63

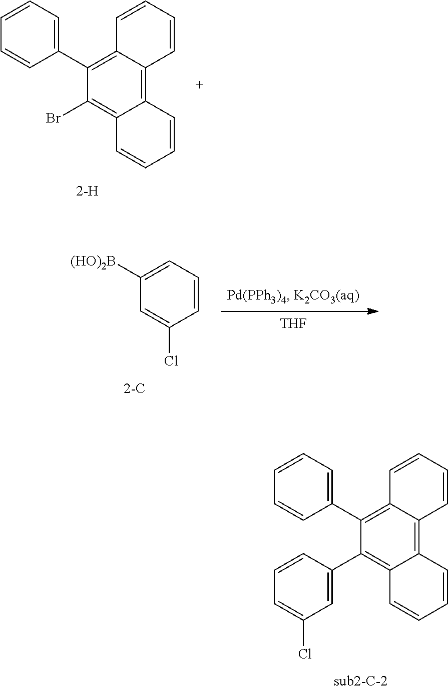 [SMILES: Brc1c(-c2ccccc2)c2ccccc2c2ccccc12 ; OB(O)c1cccc(Cl)c1 ; Clc1cccc(-c2c(-c3ccccc3)c3ccccc3c3ccccc23)c1 ; "THF"]

Step 1) Synthesis of Compound sub2-C-2

Under a nitrogen atmosphere, Compound 2-H (15 g, 45 mmol) and Compound 2-C (7.7 g, 49.5 mmol) were added to 300 mL of tetrahydrofuran (THF), and the mixture was stirred and refluxed. Afterwards, potassium carbonate (12.4 g, 90 mmol) was dissolved in 37 ml of water, and then added thereto. Thereafter, it was stirred sufficiently, followed by adding Tetrakis(triphenylphosphine) palladium (0) (1 g, 0.9 mmol). After a reaction time of 11 hours, cooling was performed to room temperature. Then, the organic layer was separated from the water layer, and then the organic layer was distilled to remove the solvent. Thereafter, it was dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-C-2 of 12.3 g. (Yield 75%, MS: [M+H]⁺=365).

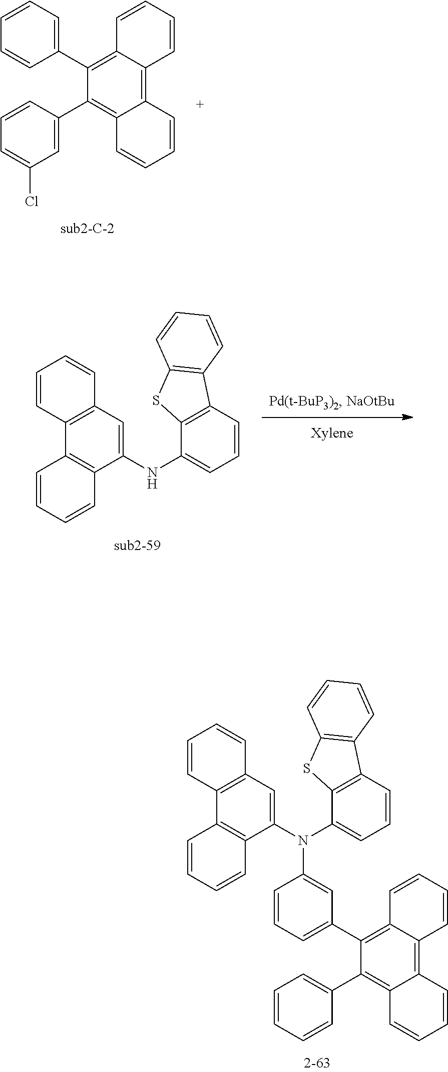 [SMILES: Clc1cccc(-c2c(-c3ccccc3)c3ccccc3c3ccccc23)c1 ; c1ccc2c(c1)cc(Nc1cccc3c1sc1ccccc13)c1ccccc12 ; c1ccc(-c2c(-c3cccc(N(c4cc5ccccc5c5ccccc45)c4cccc5c4sc4ccccc45)c3)c3ccccc3c3ccccc23)cc1]

### Step 2) Synthesis of Compound 2-63

Under a nitrogen atmosphere, Compound sub2-C-2 (10 g, 27.4 mmol) prepared in the above Step 1), Compound sub2-59 (10.3 g, 27.4 mmol), and sodium tert-butoxide (3.4 g, 35.6 mmol) were added to 200 mL of xylene, and the mixture was stirred and refluxed. Thereafter, bis(tri-tert-butylphosphine) palladium (0) (0.1 g, 0.3 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-63 of 13.5 g. (Yield 70%, MS: [M+H]⁺=704).

### Synthesis Example 2-64

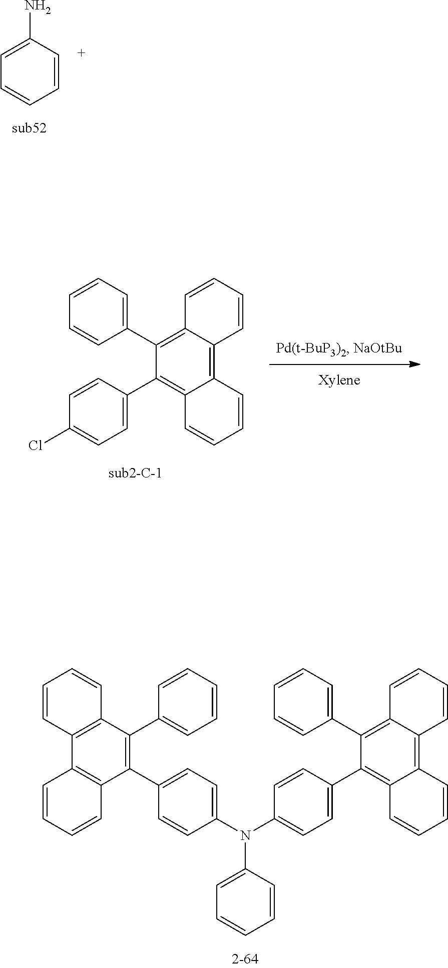 [SMILES: Nc1ccccc1 ; Clc1ccc(-c2c(-c3ccccc3)c3ccccc3c3ccccc23)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1]

Under a nitrogen atmosphere, Compound sub52 (10 g, 107.4 mmol), Compound sub2-C-1 (82.3 g, 225.5 mmol), and sodium tert-butoxide (25.8 g, 268.4 mmol) were added to 200 ml of xylene, and the mixture was stirred and refluxed. Thereafter, bis(tri-tert-butylphosphine) palladium (0) (1.1 g, 2.1 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-64 of 41 g. (Yield 51%, MS: [M+H]⁺=750).

### Synthesis Example 2-65

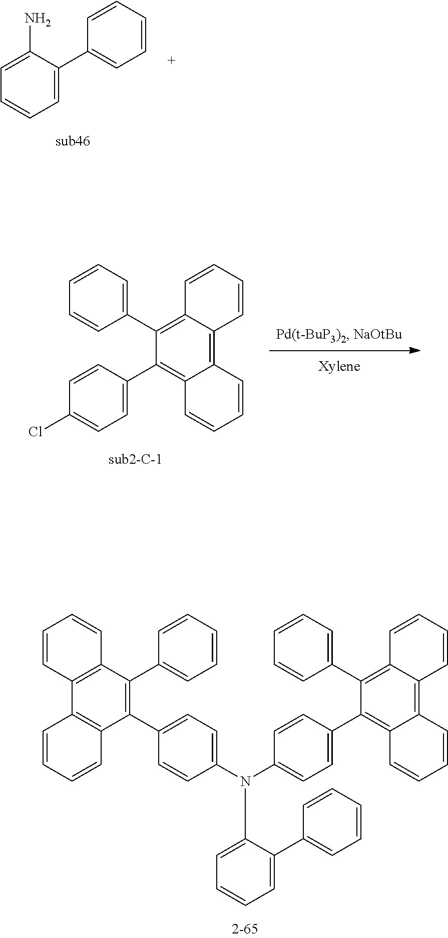 [SMILES: Nc1ccccc1-c1ccccc1 ; Clc1ccc(-c2c(-c3ccccc3)c3ccccc3c3ccccc23)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1]

Under a nitrogen atmosphere, Compound sub46 (10 g, 59.1 mmol), Compound sub2-C-1 (45.3 g, 124.1 mmol), and sodium tert-butoxide (14.2 g, 147.7 mmol) were added to 200 ml of xylene, and the mixture was stirred and refluxed. Thereafter, bis(tri-tert-butylphosphine) palladium (0) (0.6 g, 1.2 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-65 of 31.2 g. (Yield 64%, MS: [M+H]⁺=826).

### Synthesis Example 2-66

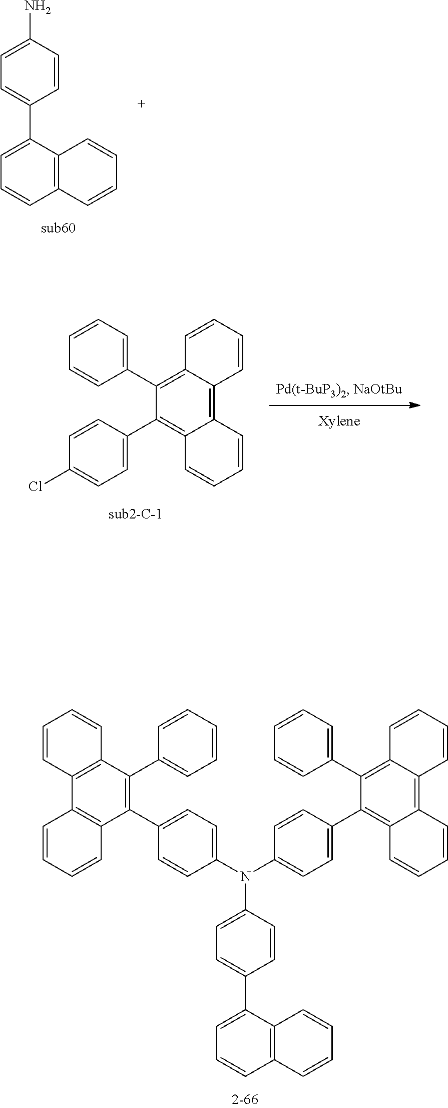 [SMILES: Nc1ccc(-c2cccc3ccccc23)cc1 ; Clc1ccc(-c2c(-c3ccccc3)c3ccccc3c3ccccc23)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1]

Under a nitrogen atmosphere, Compound sub60 (10 g, 45.6 mmol), Compound sub2-C-1 (34.9 g, 95.8 mmol), and sodium tert-butoxide (11 g, 114 mmol) were added to 200 ml of xylene, and the mixture was stirred and refluxed. Thereafter, bis(tri-tert-butylphosphine) palladium (0) (0.5 g, 0.9 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-66 of 26.7 g. (Yield 67%, MS: [M+H]⁺=876).

### Synthesis Example 2-67

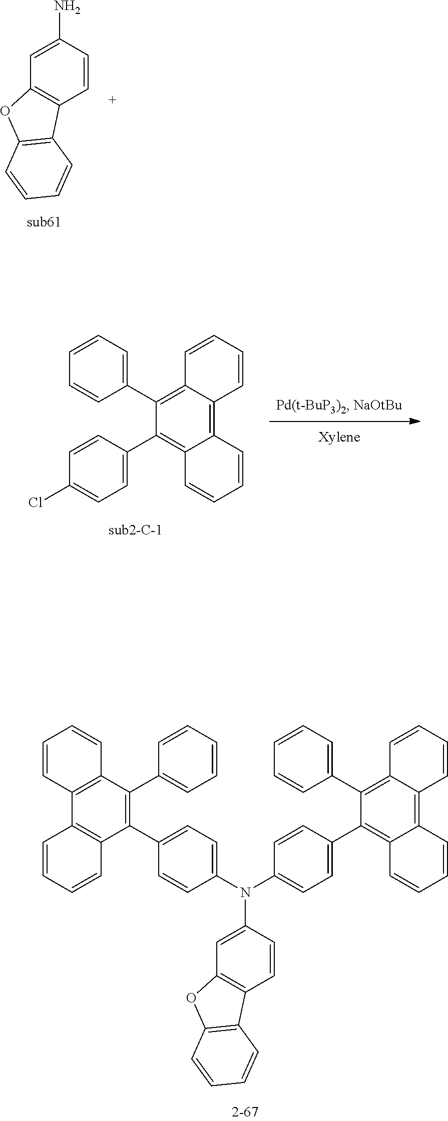 [SMILES: Nc1ccc2c(c1)oc1ccccc12 ; Clc1ccc(-c2c(-c3ccccc3)c3ccccc3c3ccccc23)cc1 ; c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1]

Under a nitrogen atmosphere, Compound sub61 (10 g, 54.6 mmol), Compound sub2-C-1 (41.8 g, 114.6 mmol), and sodium tert-butoxide (13.1 g, 136.5 mmol) were added to 200 ml of xylene, and the mixture was stirred and refluxed. Thereafter, bis(tri-tert-butylphosphine) palladium (0) (0.6 g, 1.1 mmol) was added to the mixture. The reaction was completed after 5 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-67 of 32.1 g. (Yield 70%, MS: [M+H]⁺=840).

### Synthesis Example 2-68

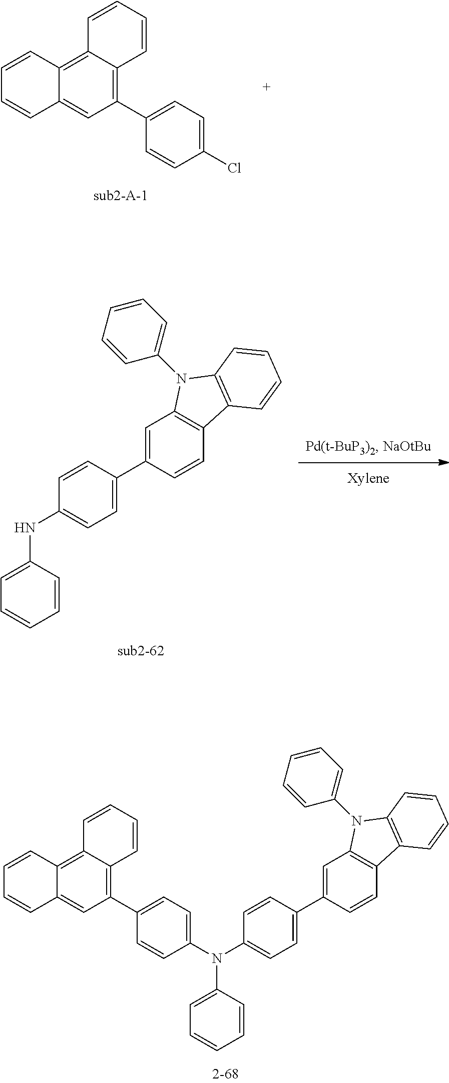 [SMILES: Clc1ccc(-c2cc3ccccc3c3ccccc23)cc1 ; c1ccc(Nc2ccc(-c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cc4ccccc4c4ccccc34)cc2)cc1]

Under a nitrogen atmosphere, Compound sub2-A-1 (10 g, 34.6 mmol), Compound sub2-62 (15.6 g, 38.1 mmol), and sodium tert-butoxide (22.1 g, 103.9 mmol) were added to 200 ml of xylene, and the mixture was stirred and refluxed. Thereafter, bis(tri-tert-butylphosphine) palladium (0) (0.4 g, 0.7 mmol) was added to the mixture. The reaction was completed after 2 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-68 of 12.6 g. (Yield 55%, MS: [M+H]⁺=663).

### Synthesis Example 2-69

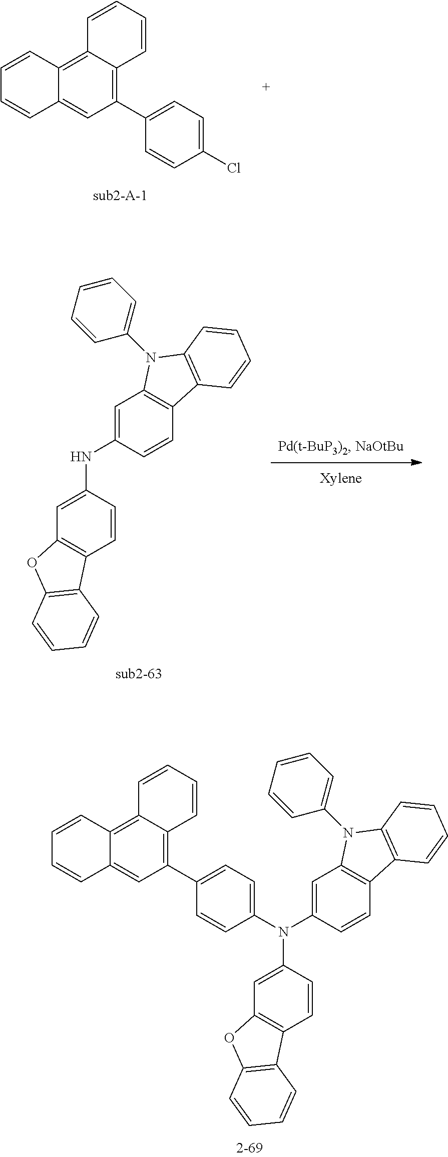 [SMILES: Clc1ccc(-c2cc3ccccc3c3ccccc23)cc1 ; c1ccc(-n2c3ccccc3c3ccc(Nc4ccc5c(c4)oc4ccccc45)cc32)cc1 ; c1ccc(-n2c3ccccc3c3ccc(N(c4ccc(-c5cc6ccccc6c6ccccc56)cc4)c4ccc5c(c4)oc4ccccc45)cc32)cc1]

Under a nitrogen atmosphere, Compound sub2-A-1 (10 g, 34.6 mmol), Compound sub2-63 (16.2 g, 38.1 mmol), and sodium tert-butoxide (22.1 g, 103.9 mmol) were added to 200 ml of xylene, and the mixture was stirred and refluxed. Thereafter, bis(tri-tert-butylphosphine) palladium (0) (0.4 g, 0.7 mmol) was added to the mixture. The reaction was completed after 2 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-69 of 12.6 g. (Yield 54%, MS: [M+H]⁺=677).

### Synthesis Example 2-70

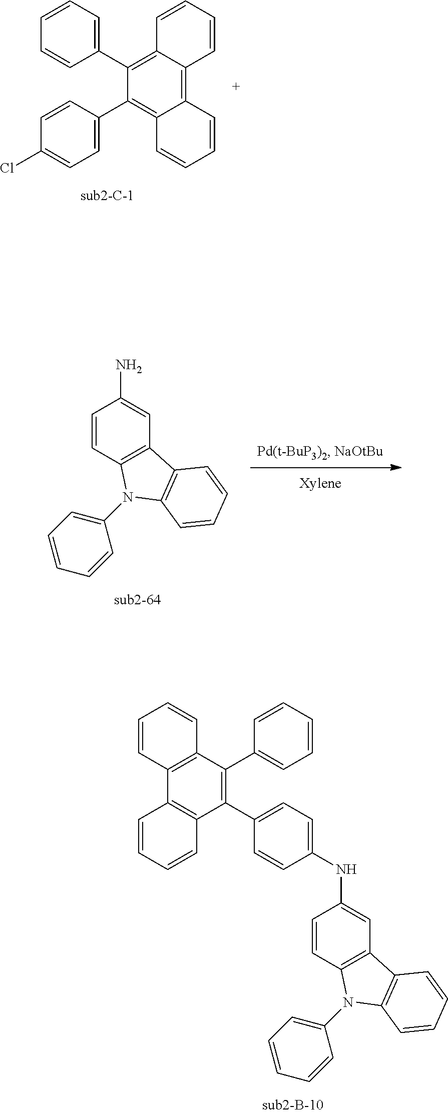 [SMILES: Clc1ccc(-c2c(-c3ccccc3)c3ccccc3c3ccccc23)cc1 ; Nc1ccc2c(c1)c1ccccc1n2-c1ccccc1 ; c1ccc(-c2c(-c3ccc(Nc4ccc5c(c4)c4ccccc4n5-c4ccccc4)cc3)c3ccccc3c3ccccc23)cc1]

Step 1) Synthesis of Compound sub2-B-10

Under a nitrogen atmosphere, Compound sub2-C-1 (10 g, 27.4 mmol), Compound sub2-64 (7.8 g, 30.1 mmol), and sodium tert-butoxide (17.5 g, 82.2 mmol) were added to 200 mL of xylene, and the mixture was stirred and refluxed. Thereafter, bis(tri-tert-butylphosphine) palladium (0) (0.3 g, 0.5 mmol) was added to the mixture. The reaction was completed after 2 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound sub2-B-10 of 11.2 g. (Yield 70%, MS: [M+H]⁺ 587).

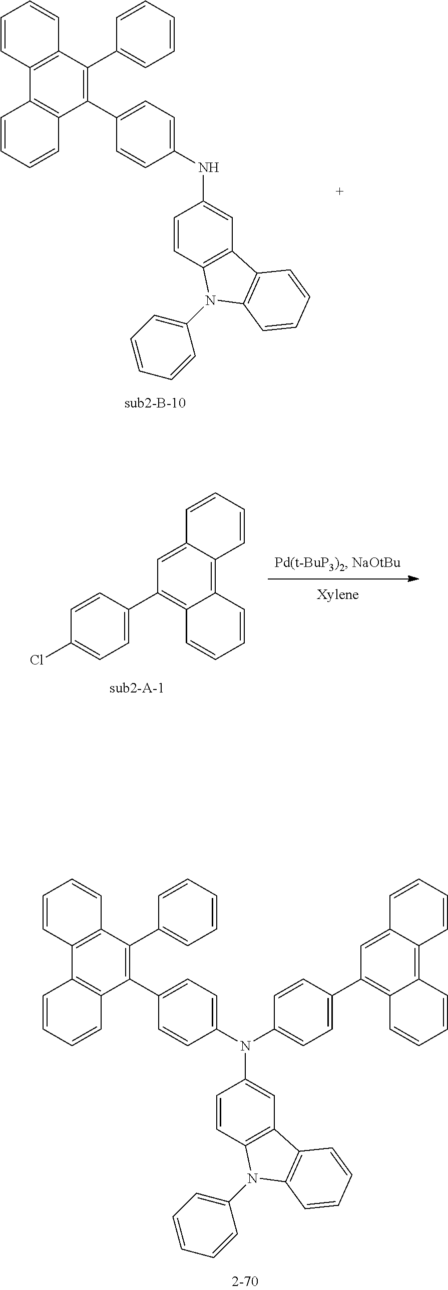 [SMILES: c1ccc(-c2c(-c3ccc(Nc4ccc5c(c4)c4ccccc4n5-c4ccccc4)cc3)c3ccccc3c3ccccc23)cc1 ; Clc1ccc(-c2cc3ccccc3c3ccccc23)cc1 ; c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1]

### Step 2) Synthesis of Compound 2-70

Under a nitrogen atmosphere, Compound sub2-B-10 (10 g, 17 mmol) prepared in the above Step 1), Compound sub2-A-1 (5.4 g, 18.7 mmol), and sodium tert-butoxide (10.9 g, 51.1 mmol) were added to 200 ml of xylene, and the mixture was stirred and refluxed. Thereafter, bis(tri-tert-butylphosphine) palladium (0) (0.2 g, 0.3 mmol) was added to the mixture. The reaction was completed after 3 hours, cooled to room temperature, and distilled under reduced pressure to remove the solvent. Then, the resulting reaction product was completely dissolved in chloroform, and washed twice with water. Afterwards, the organic layer was separated, and anhydrous magnesium sulfate was added and stirred, then filtered. The filtrate was distilled under reduced pressure. The concentrated compound was purified by silica gel column chromatography to prepare Compound 2-70 of 8.7 g. (Yield 61%, MS: [M+H]⁺=839).

### Example 1

A glass substrate on which ITO (Indium Tin Oxide) was coated as a thin film to a thickness of 1000 Å (angstrom) was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. The substrate was cleaned for 5 minutes using oxygen plasma and then transferred to a vacuum depositor.

On the prepared ITO transparent electrode, the following Compound HI-1 was thermally vacuum-deposited to a thickness of 1150 Å to form a hole injection layer, and the following compound A-1 was p-doped at a concentration of 1.5%. Then, only the following Compound HT-1 was deposited to a thickness of 800 Å to form a hole transport layer. On the hole transport layer, the following Compound EB-1 was thermally vacuum-deposited to a thickness of 150 Å to form an electron blocking layer.

On the deposition layer of the compound EB-1 as the electron blocking layer, the Compound 1-1 and the Compound 2-1 as a host compound and the following Compound Dp-7 as a dopant compound were vacuum-deposited to a thickness of 400 Å at a weight ratio of 49:49:2 to form a red light emitting layer.

On the light emitting layer, the following Compound HB-1 was vacuum-deposited to a thickness of 30 Å to form a hole blocking layer. On the hole blocking layer, the following Compound ET-1 and the following Compound LiQ were thermally vacuum-deposited to a thickness of 300 Å at a weight ratio of 2:1 to form an electron injection and transport layer. Then, on the electron injection and transport layer, lithium fluoride (LiF) with a thickness of 12 Å and aluminum with a thickness of 1000 Å was sequentially vacuum-deposited to form a cathode, thereby manufacturing an organic light emitting device.

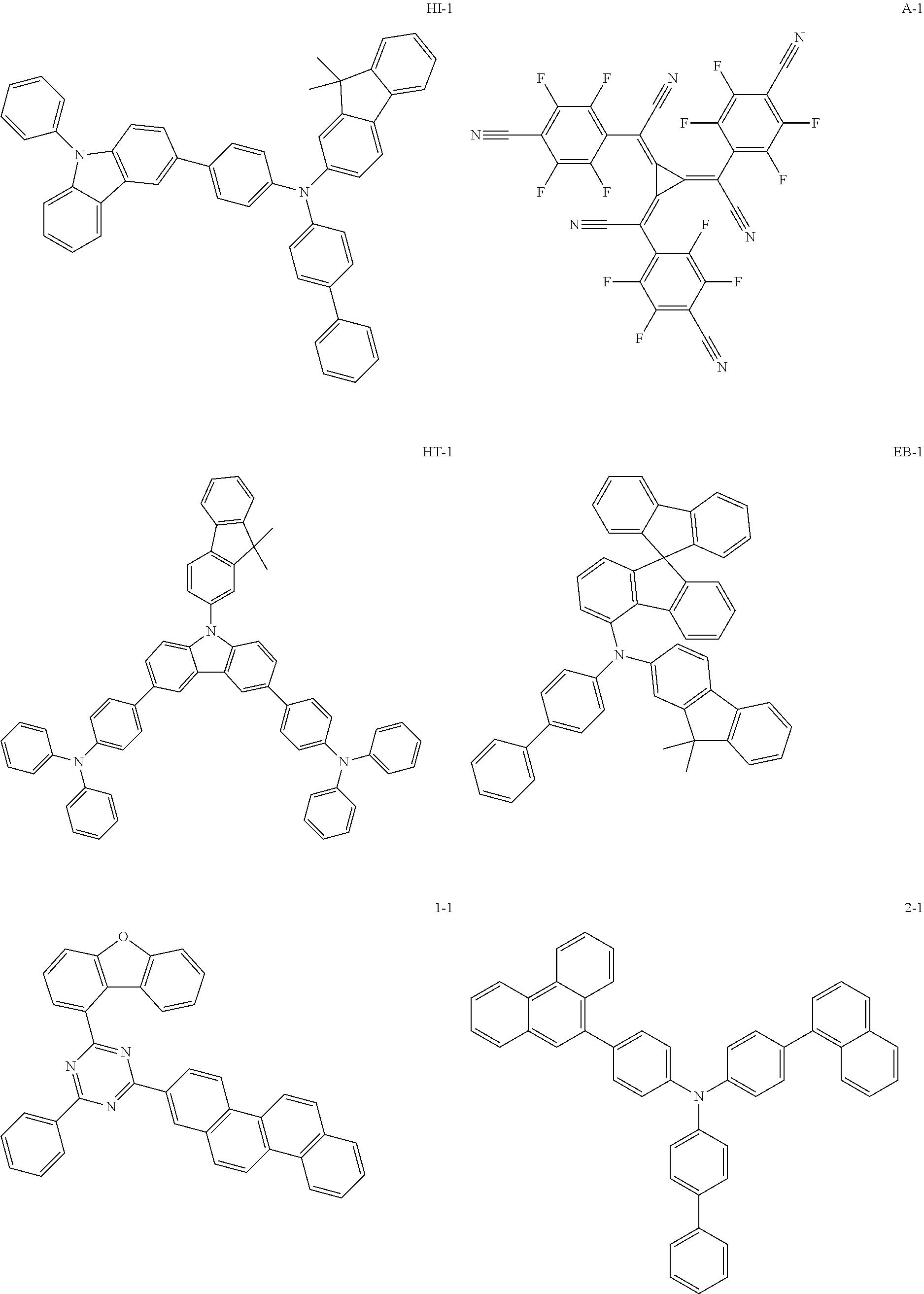

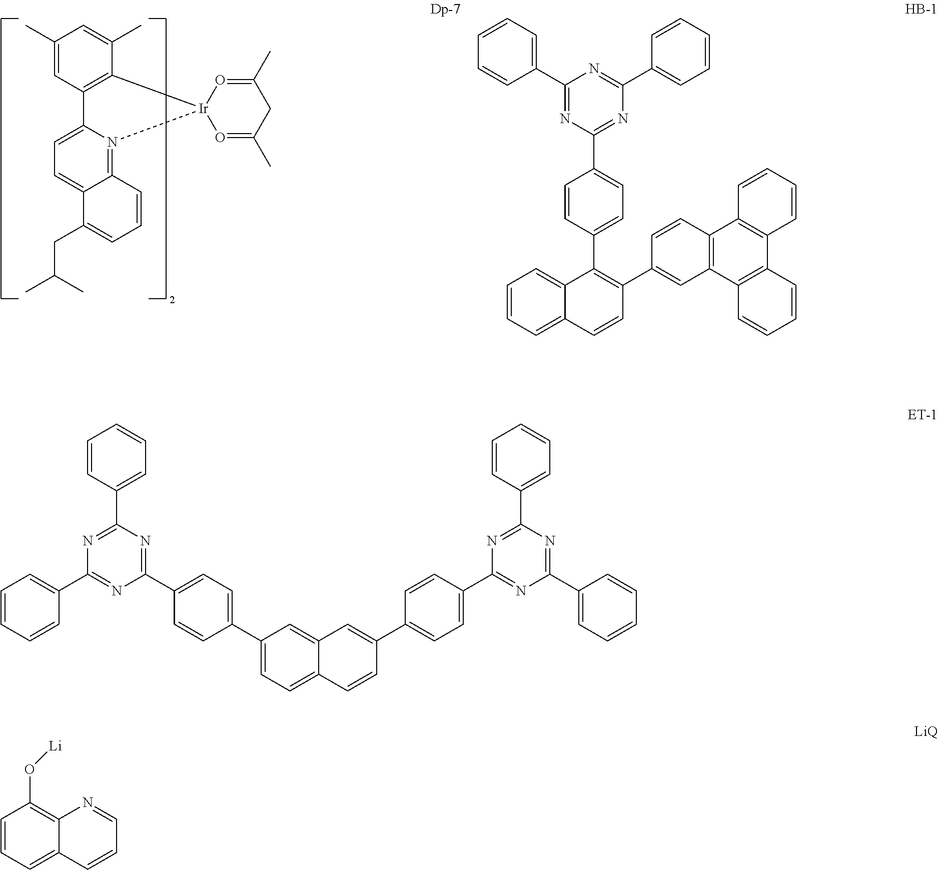

In the above process, the deposition rate of the organic material was maintained at 0.4 Å/sec to 0.7 Å/sec, the deposition rate of lithium fluoride of the cathode was maintained at 0.3 Å/sec, and the deposition rate of aluminum was maintained at 2 Å/sec. In addition, the degree of vacuum during the deposition was maintained at 2×10⁻⁷ torr to 5×10⁻⁶ torr, thereby manufacturing an organic light emitting device.

### Example 2 to 395

An organic light emitting device was manufactured in the same manner as in Example 1, except that the compound of Chemical Formula 1 as a first host compound and the compound of Chemical Formula 2 as a second host compound were used by co-deposition at a weight ratio of 1:1 as shown in Table 1 below instead of Compound 1-1 as a first host and Compound 2-1 as a second host in the organic light emitting device of Example 1.

Here, as shown in Table 1 below, the structures of the compounds used in Examples is summarized as follows.

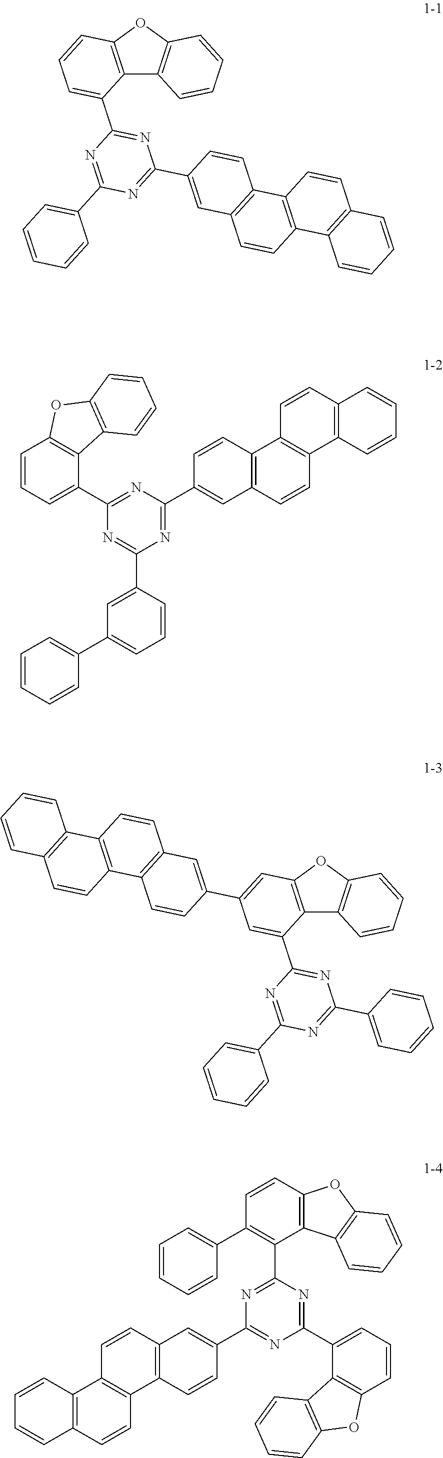 [SMILES: c1ccc(-c2nc(-c3ccc4c(ccc5c6ccccc6ccc45)c3)nc(-c3cccc4oc5ccccc5c34)n2)cc1 ; c1ccc(-c2cccc(-c3nc(-c4ccc5c(ccc6c7ccccc7ccc56)c4)nc(-c4cccc5oc6ccccc6c45)n3)c2)cc1 ; c1ccc(-c2nc(-c3ccccc3)nc(-c3cc(-c4ccc5c(ccc6c7ccccc7ccc56)c4)cc4oc5ccccc5c34)n2)cc1 ; c1ccc(-c2ccc3oc4ccccc4c3c2-c2nc(-c3ccc4c(ccc5c6ccccc6ccc45)c3)nc(-c3cccc4oc5ccccc5c34)n2)cc1]

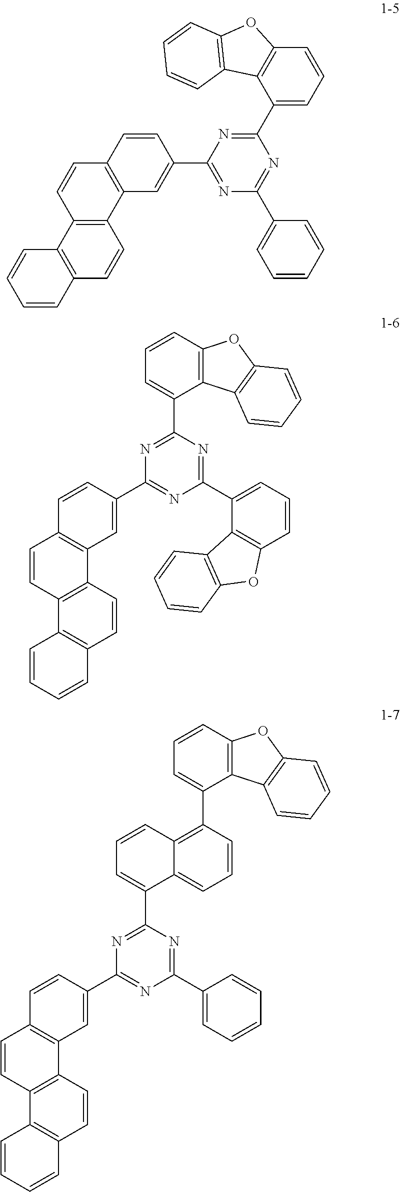 [SMILES: c1ccc(-c2nc(-c3ccc4ccc5c6ccccc6ccc5c4c3)nc(-c3cccc4oc5ccccc5c34)n2)cc1 ; c1ccc2c(c1)ccc1c3cc(-c4nc(-c5cccc6oc7ccccc7c56)nc(-c5cccc6oc7ccccc7c56)n4)ccc3ccc21 ; c1ccc(-c2nc(-c3ccc4ccc5c6ccccc6ccc5c4c3)nc(-c3cccc4c(-c5cccc6oc7ccccc7c56)cccc34)n2)cc1]

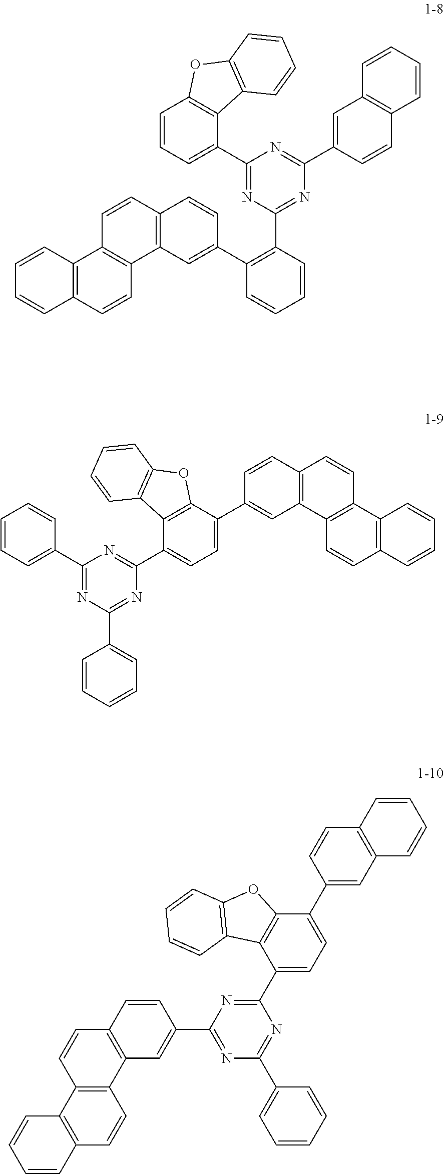 [SMILES: c1ccc(-c2nc(-c3ccc4ccccc4c3)nc(-c3cccc4oc5ccccc5c34)n2)c(-c2ccc3ccc4c5ccccc5ccc4c3c2)c1 ; c1ccc(-c2nc(-c3ccccc3)nc(-c3ccc(-c4ccc5ccc6c7ccccc7ccc6c5c4)c4oc5ccccc5c34)n2)cc1 ; c1ccc(-c2nc(-c3ccc4ccc5c6ccccc6ccc5c4c3)nc(-c3ccc(-c4ccc5ccccc5c4)c4oc5ccccc5c34)n2)cc1]

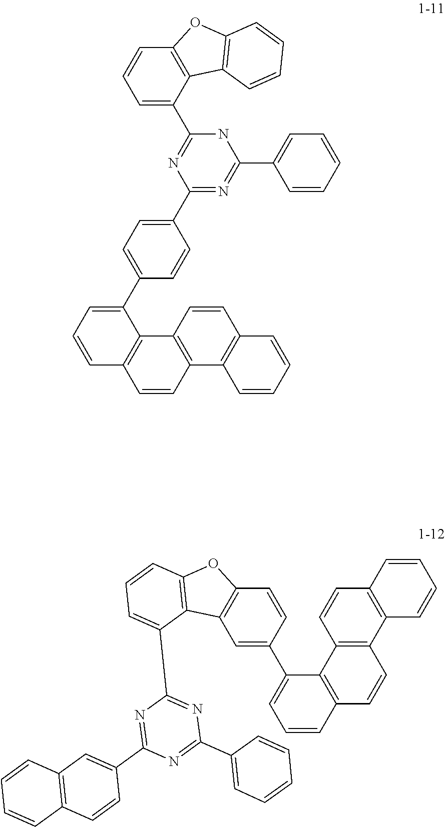

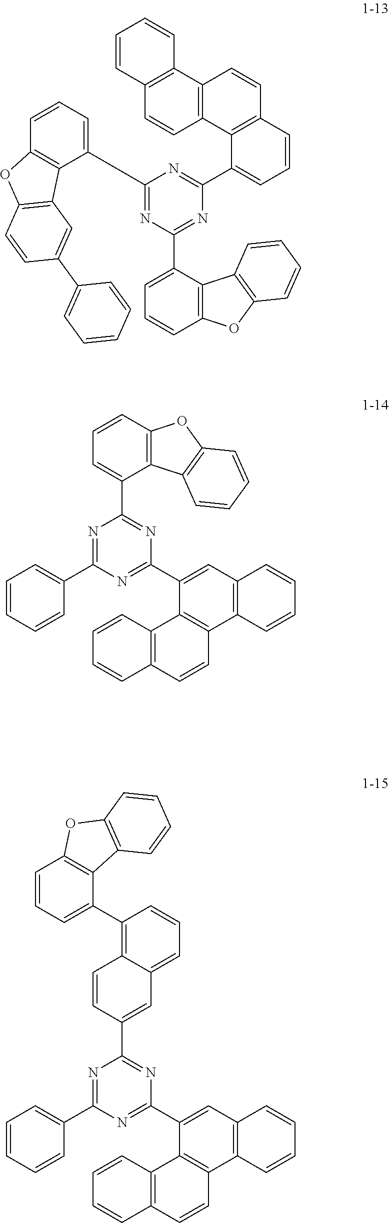 [SMILES: c1ccc(-c2ccc3oc4cccc(-c5nc(-c6cccc7ccc8c9ccccc9ccc8c67)nc(-c6cccc7oc8ccccc8c67)n5)c4c3c2)cc1 ; c1ccc(-c2nc(-c3cccc4oc5ccccc5c34)nc(-c3cc4ccccc4c4ccc5ccccc5c34)n2)cc1 ; c1ccc(-c2nc(-c3ccc4c(-c5cccc6oc7ccccc7c56)cccc4c3)nc(-c3cc4ccccc4c4ccc5ccccc5c34)n2)cc1]

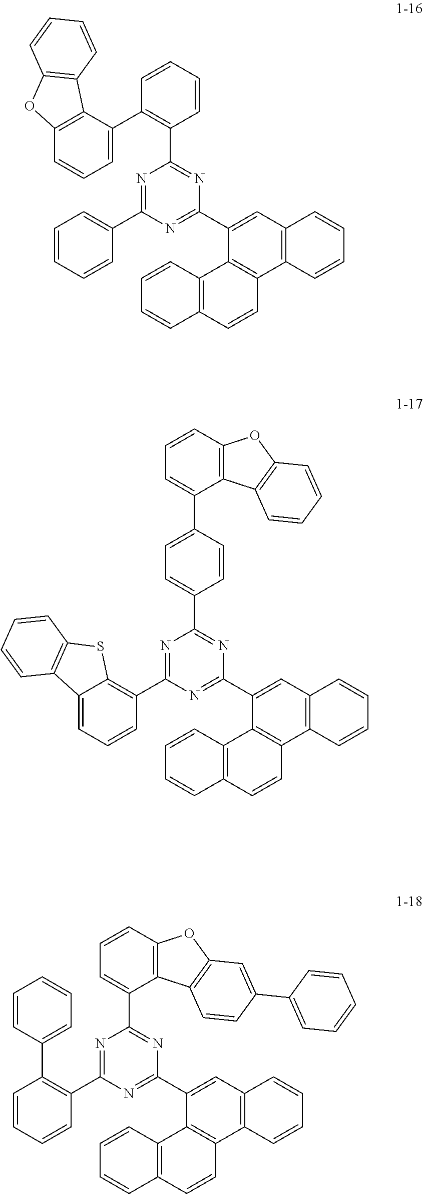 [SMILES: c1ccc(-c2nc(-c3ccccc3-c3cccc4oc5ccccc5c34)nc(-c3cc4ccccc4c4ccc5ccccc5c34)n2)cc1 ; c1ccc2c(c1)cc(-c1nc(-c3ccc(-c4cccc5oc6ccccc6c45)cc3)nc(-c3cccc4c3sc3ccccc34)n1)c1c3ccccc3ccc21 ; c1ccc(-c2ccc3c(c2)oc2cccc(-c4nc(-c5ccccc5-c5ccccc5)nc(-c5cc6ccccc6c6ccc7ccccc7c56)n4)c23)cc1]

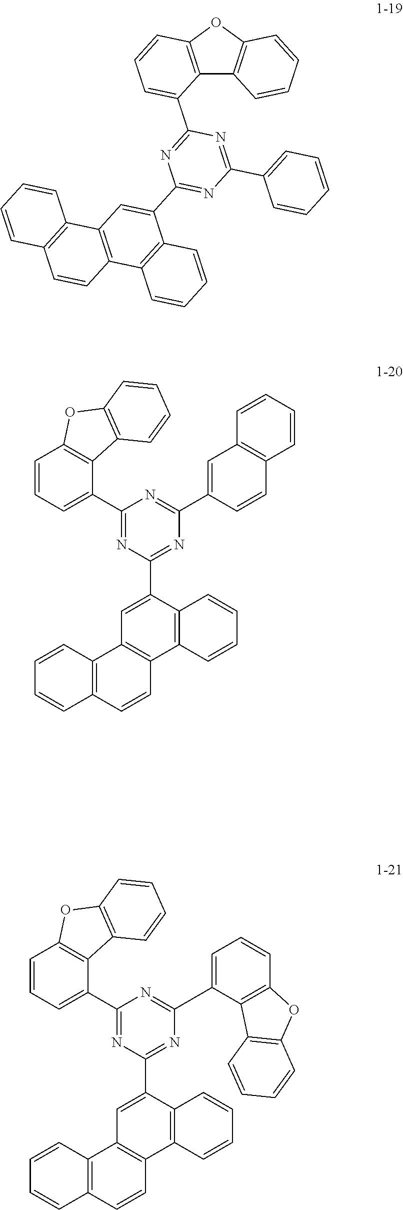 [SMILES: c1ccc(-c2nc(-c3cc4c5ccccc5ccc4c4ccccc34)nc(-c3cccc4oc5ccccc5c34)n2)cc1 ; c1ccc2cc(-c3nc(-c4cc5c6ccccc6ccc5c5ccccc45)nc(-c4cccc5oc6ccccc6c45)n3)ccc2c1 ; c1ccc2c(c1)ccc1c3ccccc3c(-c3nc(-c4cccc5oc6ccccc6c45)nc(-c4cccc5oc6ccccc6c45)n3)cc21]

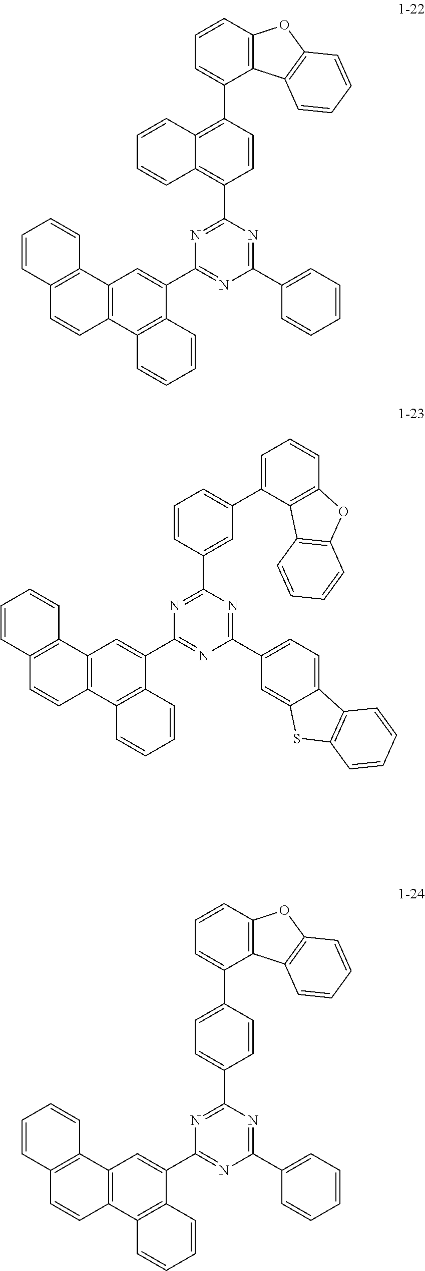 [SMILES: c1ccc(-c2nc(-c3ccc(-c4cccc5oc6ccccc6c45)c4ccccc34)nc(-c3cc4c5ccccc5ccc4c4ccccc34)n2)cc1 ; c1cc(-c2nc(-c3ccc4c(c3)sc3ccccc34)nc(-c3cc4c5ccccc5ccc4c4ccccc34)n2)cc(-c2cccc3oc4ccccc4c23)c1 ; c1ccc(-c2nc(-c3ccc(-c4cccc5oc6ccccc6c45)cc3)nc(-c3cc4c5ccccc5ccc4c4ccccc34)n2)cc1]

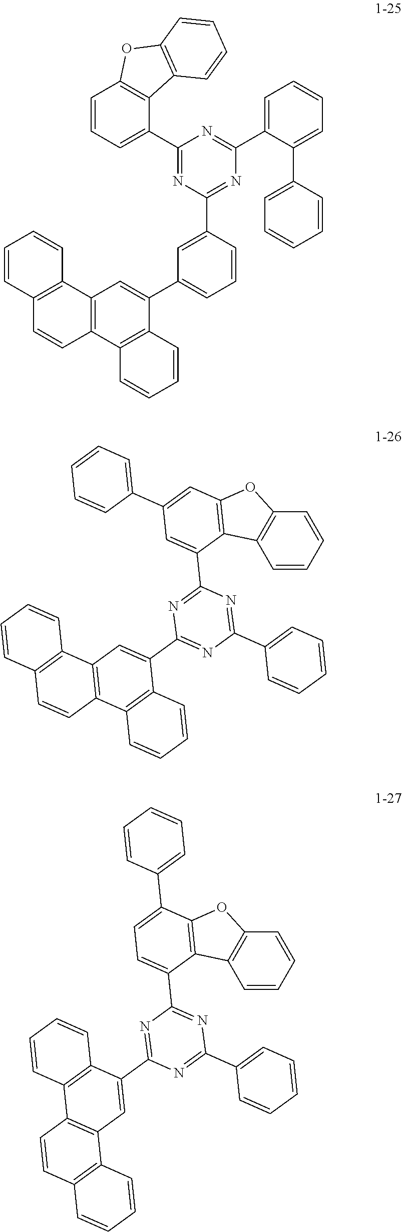 [SMILES: c1ccc(-c2ccccc2-c2nc(-c3cccc(-c4cc5c6ccccc6ccc5c5ccccc45)c3)nc(-c3cccc4oc5ccccc5c34)n2)cc1 ; c1ccc(-c2cc(-c3nc(-c4ccccc4)nc(-c4cc5c6ccccc6ccc5c5ccccc45)n3)c3c(c2)oc2ccccc23)cc1 ; c1ccc(-c2nc(-c3cc4c5ccccc5ccc4c4ccccc34)nc(-c3ccc(-c4ccccc4)c4oc5ccccc5c34)n2)cc1]

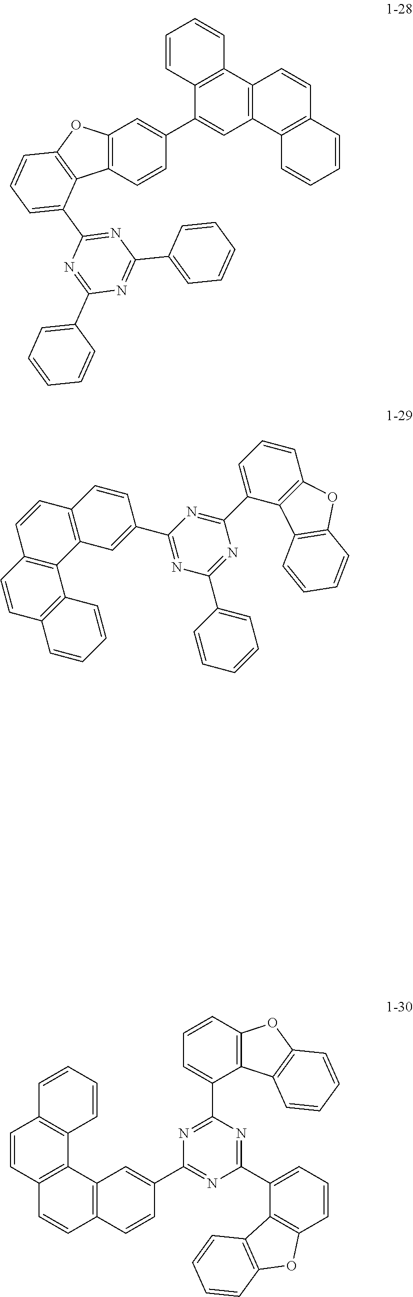 [SMILES: c1ccc(-c2nc(-c3ccccc3)nc(-c3cccc4oc5cc(-c6cc7c8ccccc8ccc7c7ccccc67)ccc5c34)n2)cc1 ; c1ccc(-c2nc(-c3ccc4ccc5ccc6ccccc6c5c4c3)nc(-c3cccc4oc5ccccc5c34)n2)cc1 ; c1ccc2c(c1)ccc1ccc3ccc(-c4nc(-c5cccc6oc7ccccc7c56)nc(-c5cccc6oc7ccccc7c56)n4)cc3c12]

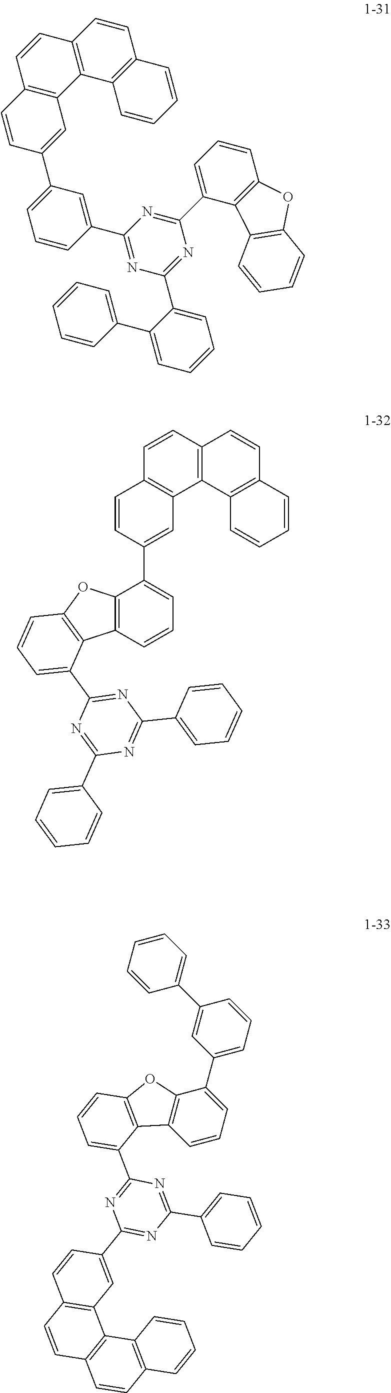 [SMILES: c1ccc(-c2ccccc2-c2nc(-c3cccc(-c4ccc5ccc6ccc7ccccc7c6c5c4)c3)nc(-c3cccc4oc5ccccc5c34)n2)cc1 ; c1ccc(-c2nc(-c3ccccc3)nc(-c3cccc4oc5c(-c6ccc7ccc8ccc9ccccc9c8c7c6)cccc5c34)n2)cc1 ; c1ccc(-c2cccc(-c3cccc4c3oc3cccc(-c5nc(-c6ccccc6)nc(-c6ccc7ccc8ccc9ccccc9c8c7c6)n5)c34)c2)cc1]

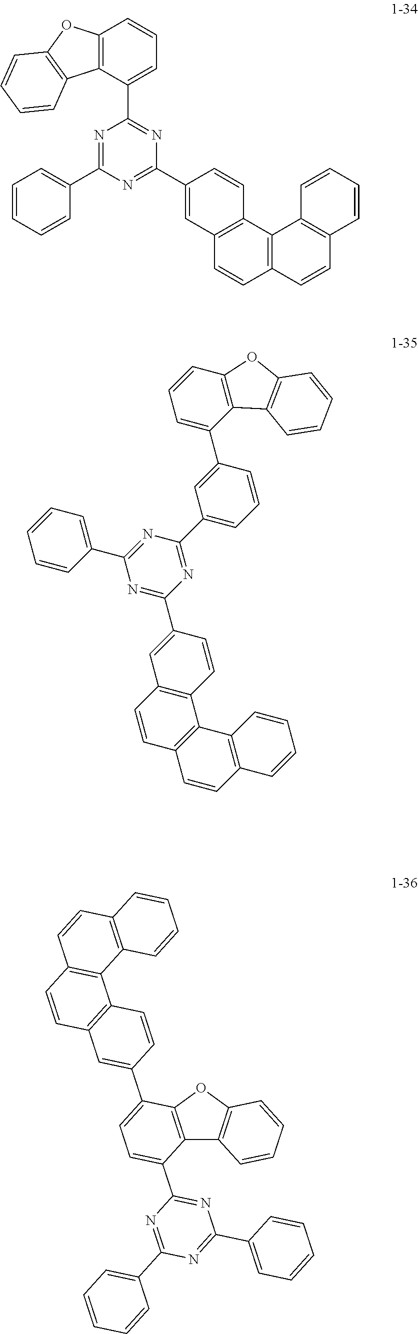 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3cccc(-c4cccc5oc6ccccc6c45)c3)nc(-c3ccc4c(ccc5ccc6ccccc6c54)c3)n2)cc1 ; c1ccc(-c2nc(-c3ccccc3)nc(-c3ccc(-c4ccc5c(ccc6ccc7ccccc7c65)c4)c4oc5ccccc5c34)n2)cc1]

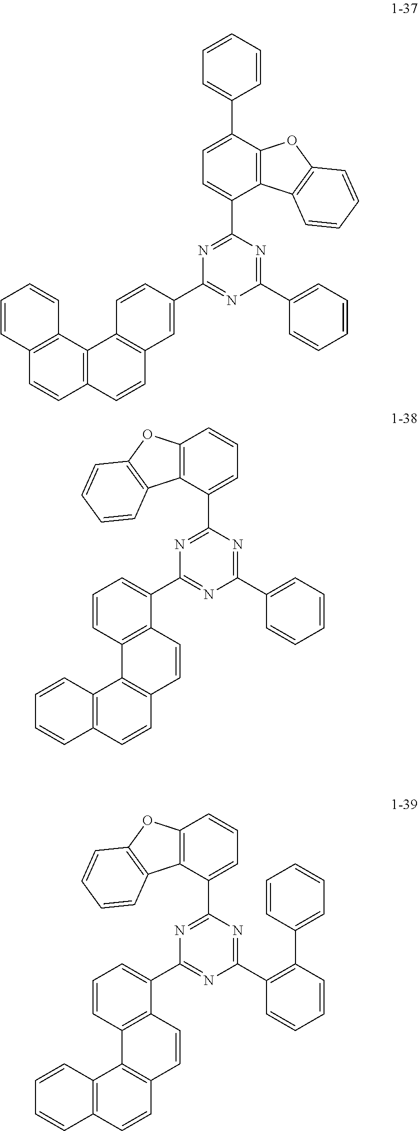

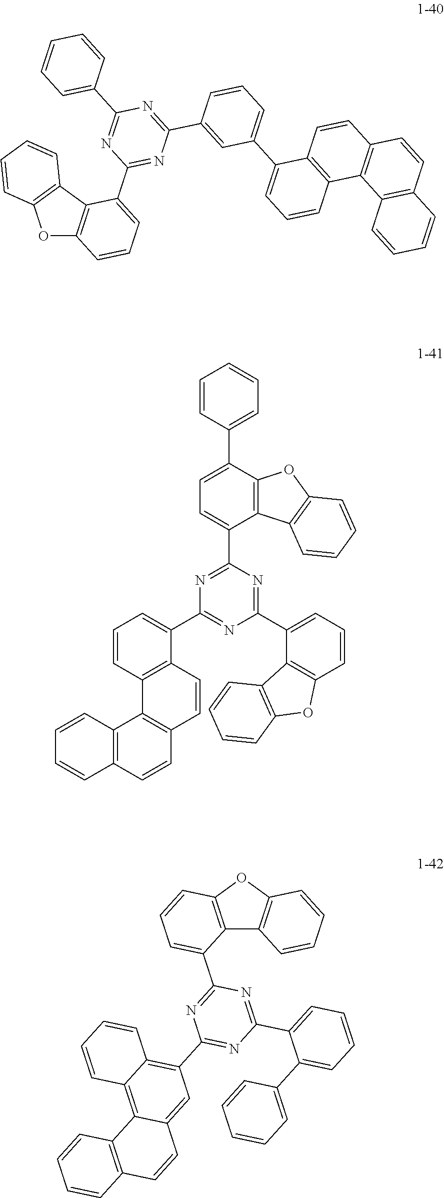 [SMILES: c1ccc(-c2nc(-c3cccc(-c4cccc5c4ccc4ccc6ccccc6c45)c3)nc(-c3cccc4oc5ccccc5c34)n2)cc1 ; c1ccc(-c2ccc(-c3nc(-c4cccc5c4ccc4ccc6ccccc6c45)nc(-c4cccc5oc6ccccc6c45)n3)c3c2oc2ccccc23)cc1 ; c1ccc(-c2ccccc2-c2nc(-c3cc4ccc5ccccc5c4c4ccccc34)nc(-c3cccc4oc5ccccc5c34)n2)cc1]

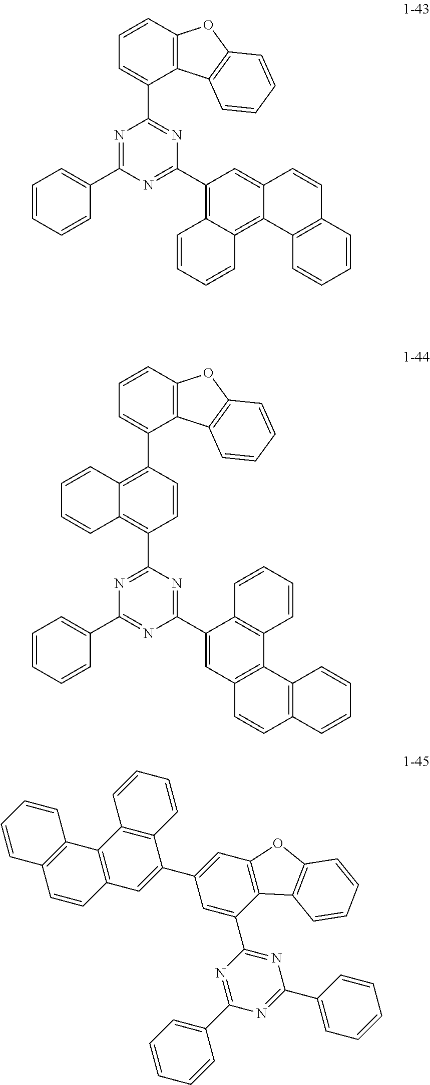 [SMILES: c1ccc(-c2nc(-c3cc4ccc5ccccc5c4c4ccccc34)nc(-c3cccc4oc5ccccc5c34)n2)cc1 ; c1ccc(-c2nc(-c3ccc(-c4cccc5oc6ccccc6c45)c4ccccc34)nc(-c3cc4ccc5ccccc5c4c4ccccc34)n2)cc1 ; c1ccc(-c2nc(-c3ccccc3)nc(-c3cc(-c4cc5ccc6ccccc6c5c5ccccc45)cc4oc5ccccc5c34)n2)cc1]

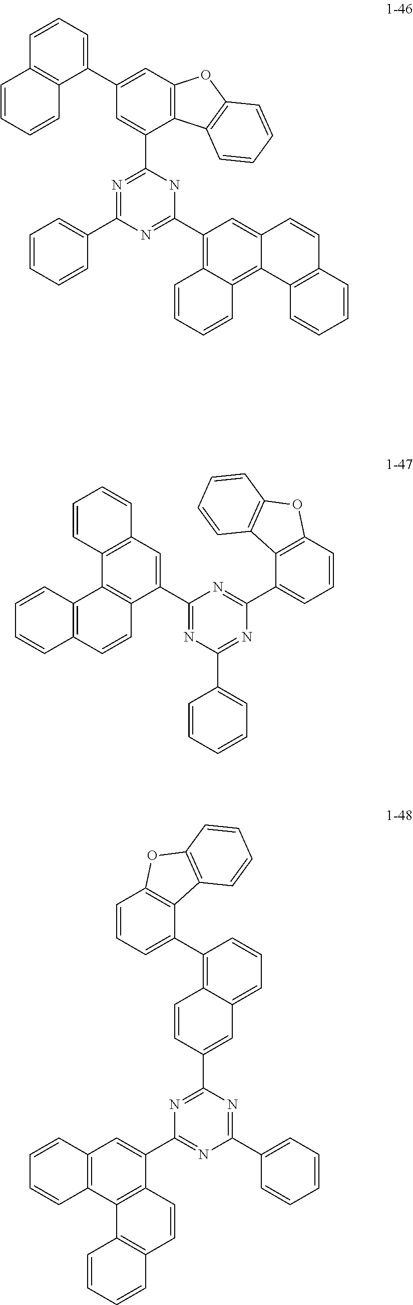

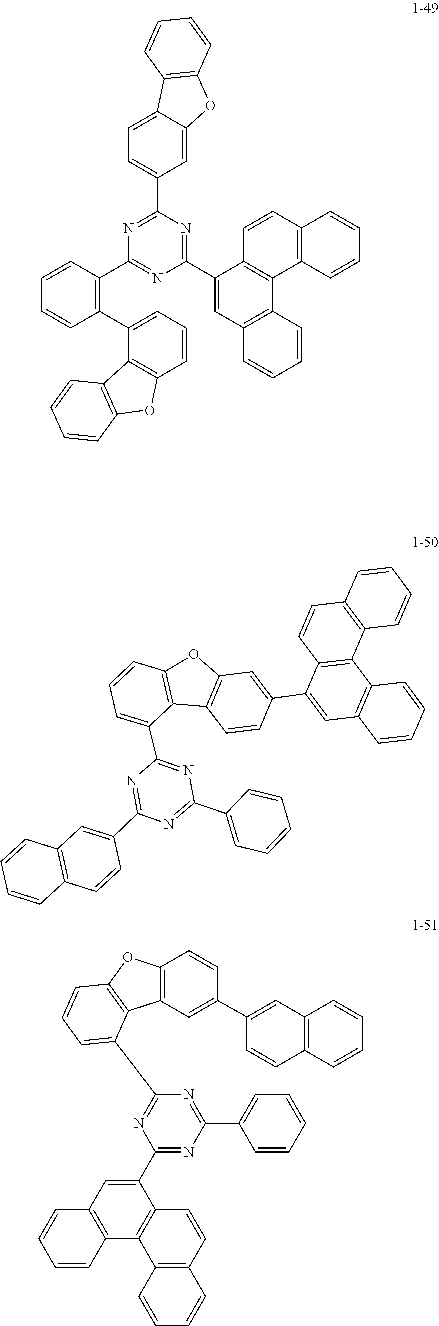 [SMILES: c1ccc(-c2cccc3oc4ccccc4c23)c(-c2nc(-c3ccc4c(c3)oc3ccccc34)nc(-c3cc4ccccc4c4c3ccc3ccccc34)n2)c1 ; c1ccc(-c2nc(-c3ccc4ccccc4c3)nc(-c3cccc4oc5cc(-c6cc7ccccc7c7c6ccc6ccccc67)ccc5c34)n2)cc1 ; c1ccc(-c2nc(-c3cc4ccccc4c4c3ccc3ccccc34)nc(-c3cccc4oc5ccc(-c6ccc7ccccc7c6)cc5c34)n2)cc1]

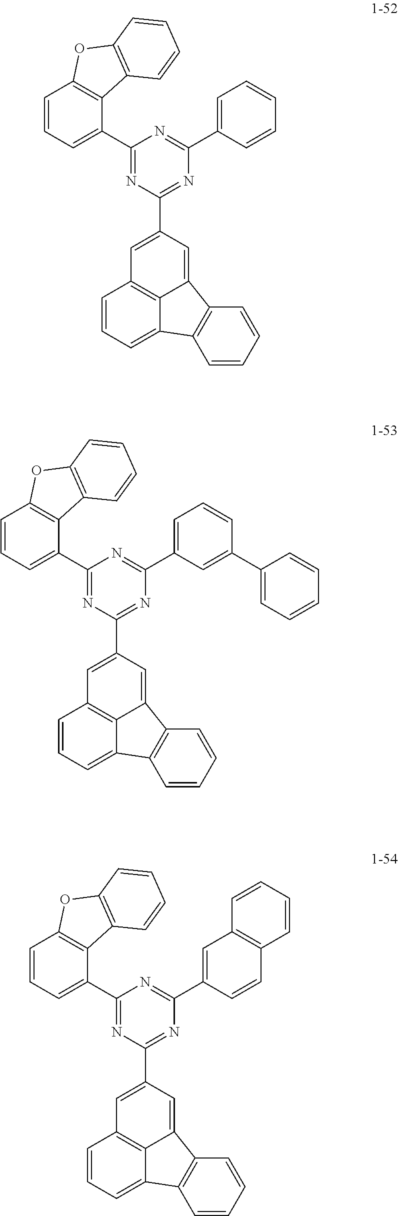 [SMILES: c1ccc(-c2nc(-c3cc4c5c(cccc5c3)-c3ccccc3-4)nc(-c3cccc4oc5ccccc5c34)n2)cc1 ; c1ccc(-c2cccc(-c3nc(-c4cc5c6c(cccc6c4)-c4ccccc4-5)nc(-c4cccc5oc6ccccc6c45)n3)c2)cc1 ; c1ccc2c(c1)-c1cccc3cc(-c4nc(-c5ccc6ccccc6c5)nc(-c5cccc6oc7ccccc7c56)n4)cc-2c13]

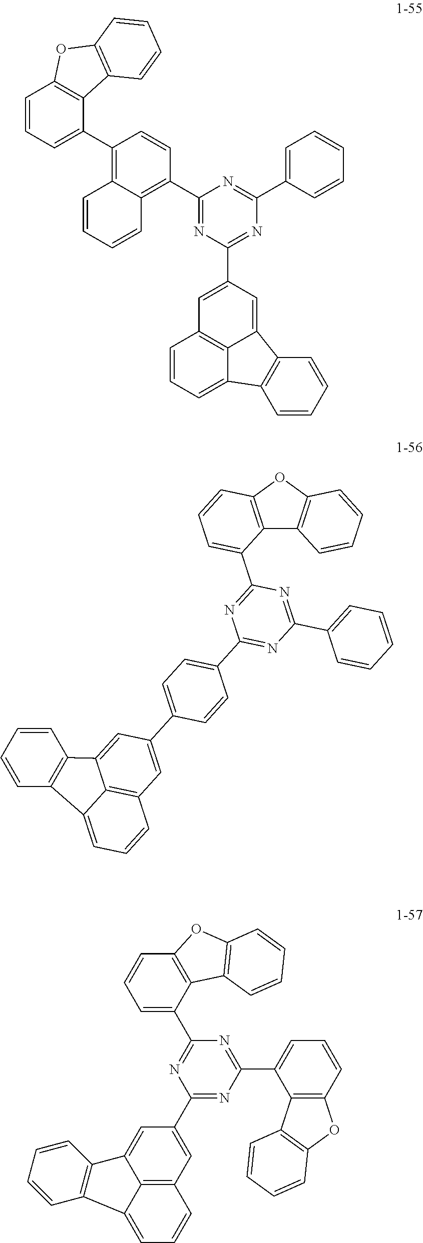 [SMILES: c1ccc(-c2nc(-c3cc4c5c(cccc5c3)-c3ccccc3-4)nc(-c3ccc(-c4cccc5oc6ccccc6c45)c4ccccc34)n2)cc1 ; c1ccc(-c2nc(-c3ccc(-c4cc5c6c(cccc6c4)-c4ccccc4-5)cc3)nc(-c3cccc4oc5ccccc5c34)n2)cc1 ; c1ccc2c(c1)-c1cccc3cc(-c4nc(-c5cccc6oc7ccccc7c56)nc(-c5cccc6oc7ccccc7c56)n4)cc-2c13]

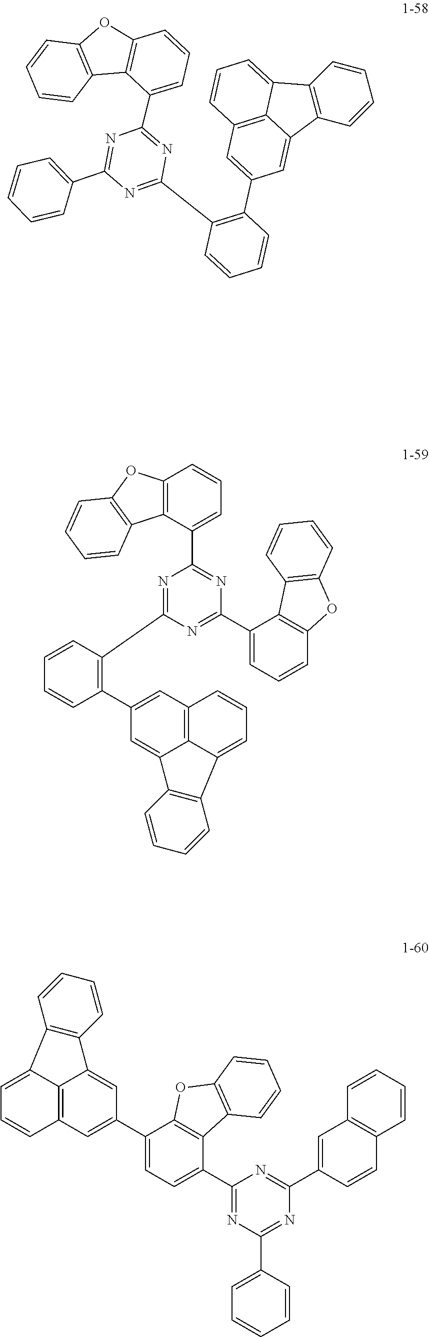 [SMILES: c1ccc(-c2nc(-c3ccccc3-c3cc4c5c(cccc5c3)-c3ccccc3-4)nc(-c3cccc4oc5ccccc5c34)n2)cc1 ; c1ccc(-c2nc(-c3cccc4oc5ccccc5c34)nc(-c3cccc4oc5ccccc5c34)n2)c(-c2cc3c4c(cccc4c2)-c2ccccc2-3)c1 ; c1ccc(-c2nc(-c3ccc4ccccc4c3)nc(-c3ccc(-c4cc5c6c(cccc6c4)-c4ccccc4-5)c4oc5ccccc5c34)n2)cc1]

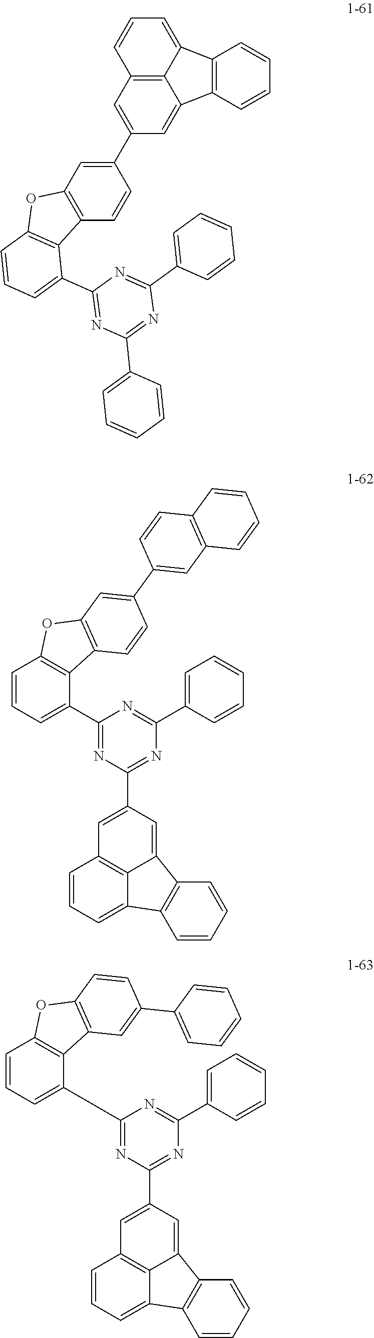 [SMILES: c1ccc(-c2nc(-c3ccccc3)nc(-c3cccc4oc5cc(-c6cc7c8c(cccc8c6)-c6ccccc6-7)ccc5c34)n2)cc1 ; c1ccc(-c2nc(-c3cc4c5c(cccc5c3)-c3ccccc3-4)nc(-c3cccc4oc5cc(-c6ccc7ccccc7c6)ccc5c34)n2)cc1 ; c1ccc(-c2ccc3oc4cccc(-c5nc(-c6ccccc6)nc(-c6cc7c8c(cccc8c6)-c6ccccc6-7)n5)c4c3c2)cc1]

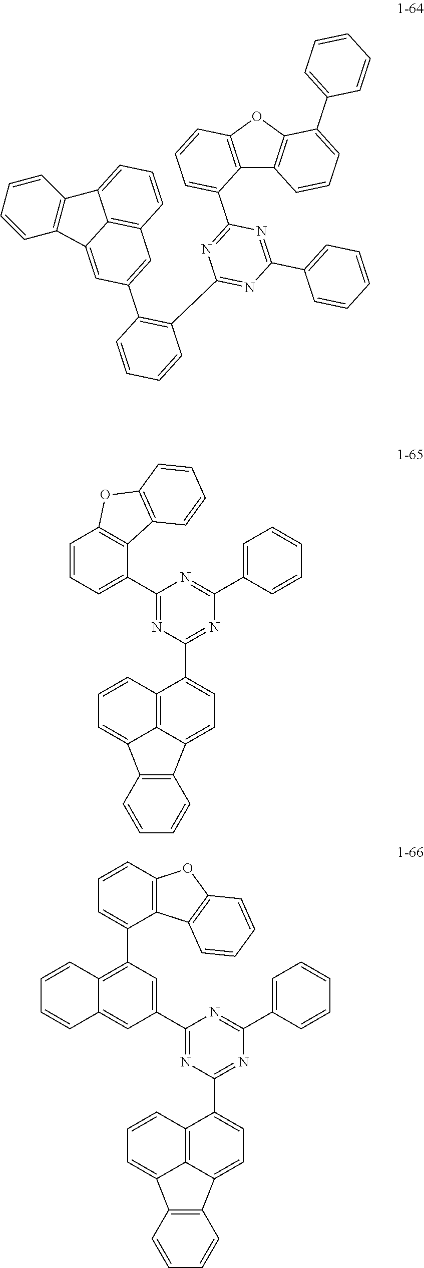

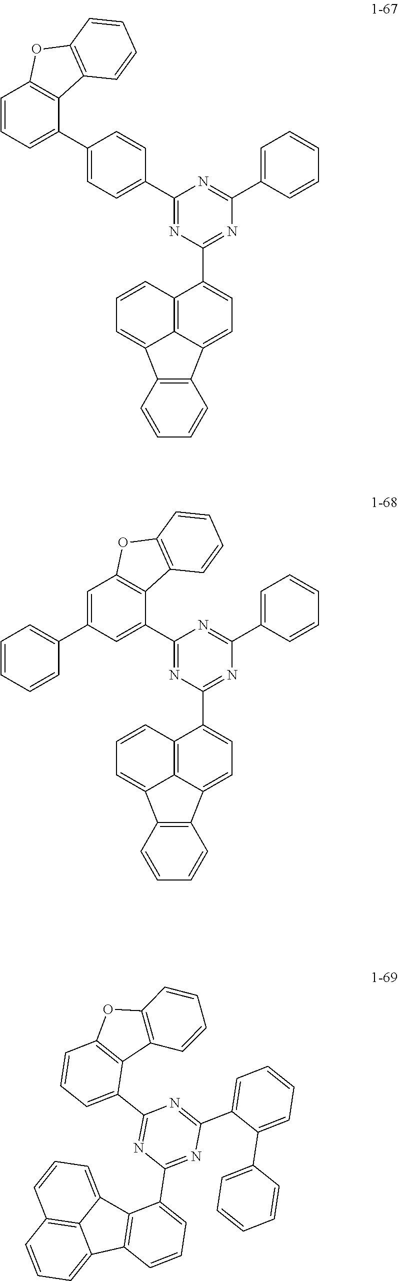 [SMILES: c1ccc(-c2nc(-c3ccc(-c4cccc5oc6ccccc6c45)cc3)nc(-c3ccc4c5c(cccc35)-c3ccccc3-4)n2)cc1 ; c1ccc(-c2cc(-c3nc(-c4ccccc4)nc(-c4ccc5c6c(cccc46)-c4ccccc4-5)n3)c3c(c2)oc2ccccc23)cc1 ; c1ccc(-c2ccccc2-c2nc(-c3cccc4c3-c3cccc5cccc-4c35)nc(-c3cccc4oc5ccccc5c34)n2)cc1]

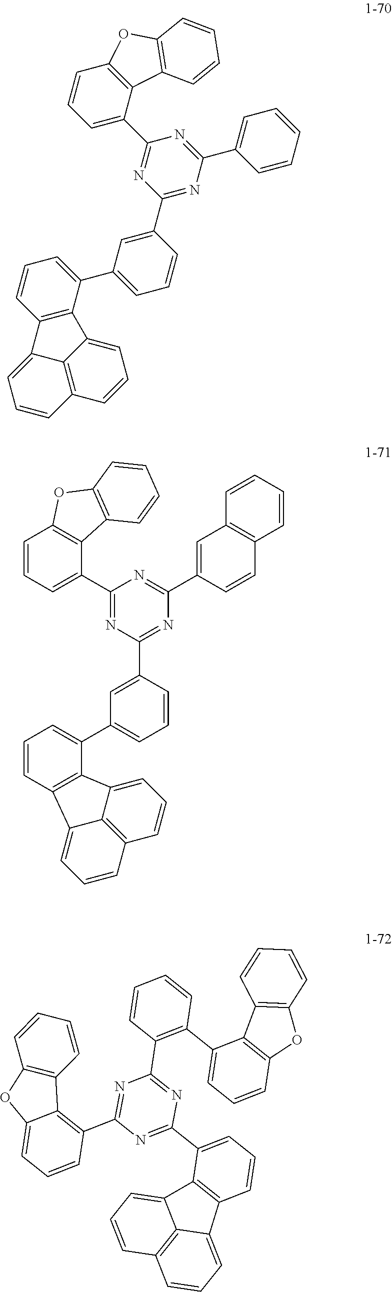 [SMILES: c1ccc(-c2nc(-c3cccc(-c4cccc5c4-c4cccc6cccc-5c46)c3)nc(-c3cccc4oc5ccccc5c34)n2)cc1 ; c1cc(-c2nc(-c3ccc4ccccc4c3)nc(-c3cccc4oc5ccccc5c34)n2)cc(-c2cccc3c2-c2cccc4cccc-3c24)c1 ; c1ccc(-c2cccc3oc4ccccc4c23)c(-c2nc(-c3cccc4c3-c3cccc5cccc-4c35)nc(-c3cccc4oc5ccccc5c34)n2)c1]

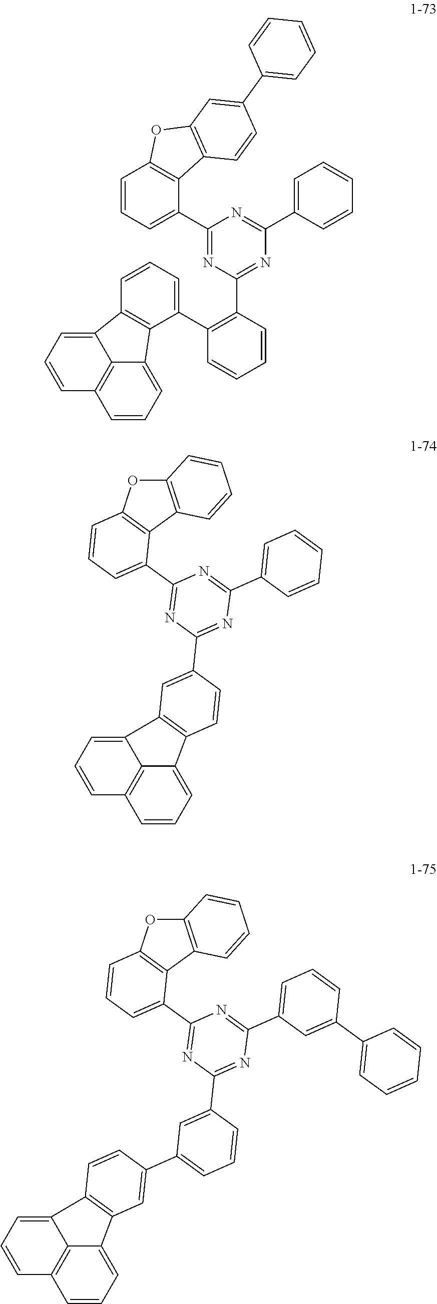 [SMILES: c1ccc(-c2ccc3c(c2)oc2cccc(-c4nc(-c5ccccc5)nc(-c5ccccc5-c5cccc6c5-c5cccc7cccc-6c57)n4)c23)cc1 ; c1ccc(-c2nc(-c3ccc4c(c3)-c3cccc5cccc-4c35)nc(-c3cccc4oc5ccccc5c34)n2)cc1 ; c1ccc(-c2cccc(-c3nc(-c4cccc(-c5ccc6c(c5)-c5cccc7cccc-6c57)c4)nc(-c4cccc5oc6ccccc6c45)n3)c2)cc1]

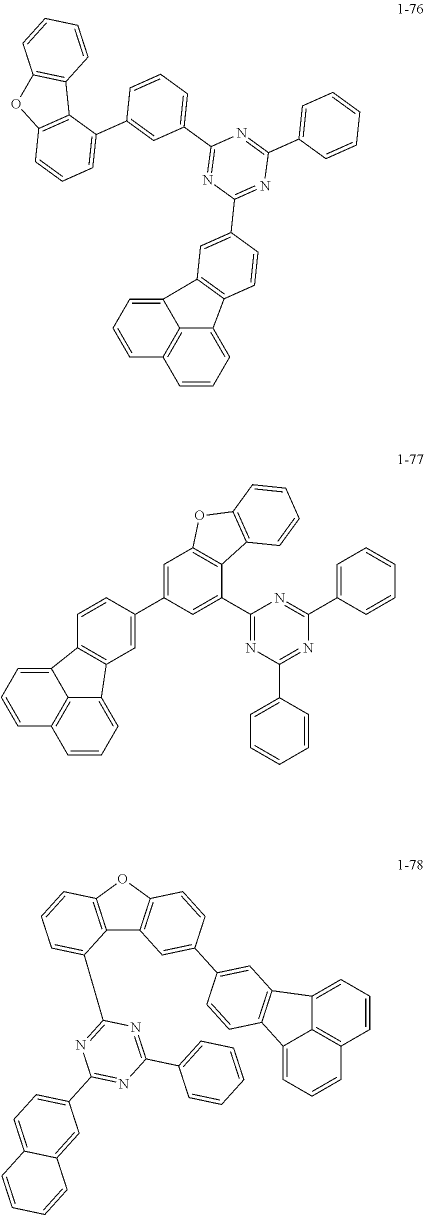 [SMILES: c1ccc(-c2nc(-c3cccc(-c4cccc5oc6ccccc6c45)c3)nc(-c3ccc4c(c3)-c3cccc5cccc-4c35)n2)cc1 ; c1ccc(-c2nc(-c3ccccc3)nc(-c3cc(-c4ccc5c(c4)-c4cccc6cccc-5c46)cc4oc5ccccc5c34)n2)cc1 ; c1ccc(-c2nc(-c3ccc4ccccc4c3)nc(-c3cccc4oc5ccc(-c6ccc7c(c6)-c6cccc8cccc-7c68)cc5c34)n2)cc1]

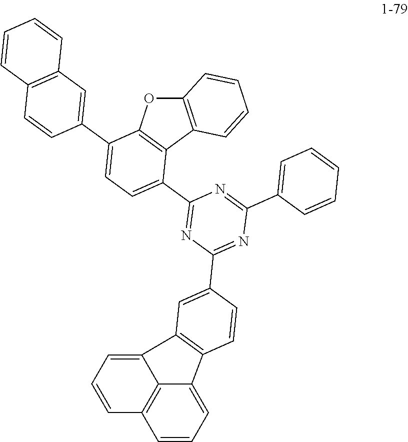 [SMILES: c1ccc(-c2nc(-c3ccc4c(c3)-c3cccc5cccc-4c35)nc(-c3ccc(-c4ccc5ccccc5c4)c4oc5ccccc5c34)n2)cc1]

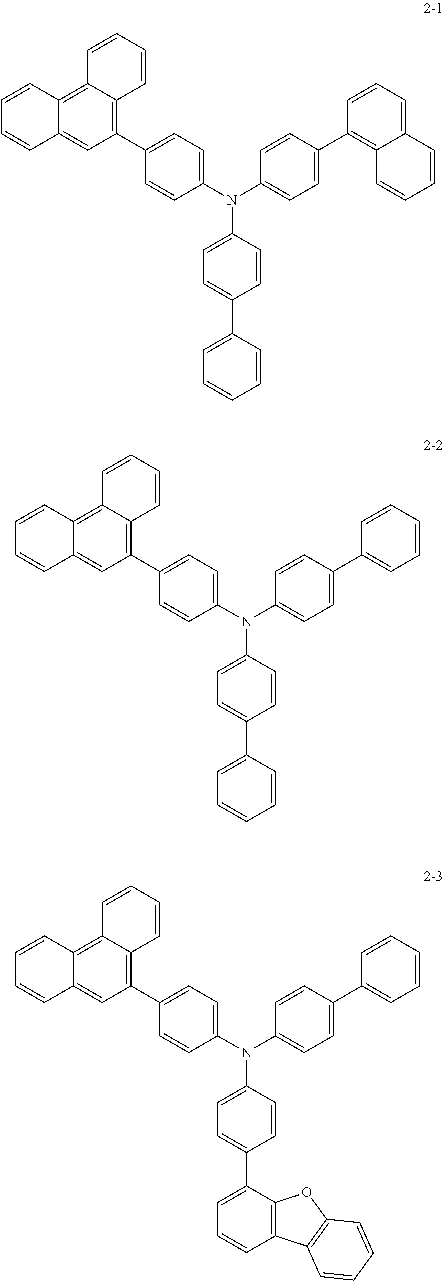 [SMILES: c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4cccc5c4oc4ccccc45)cc3)cc2)cc1]

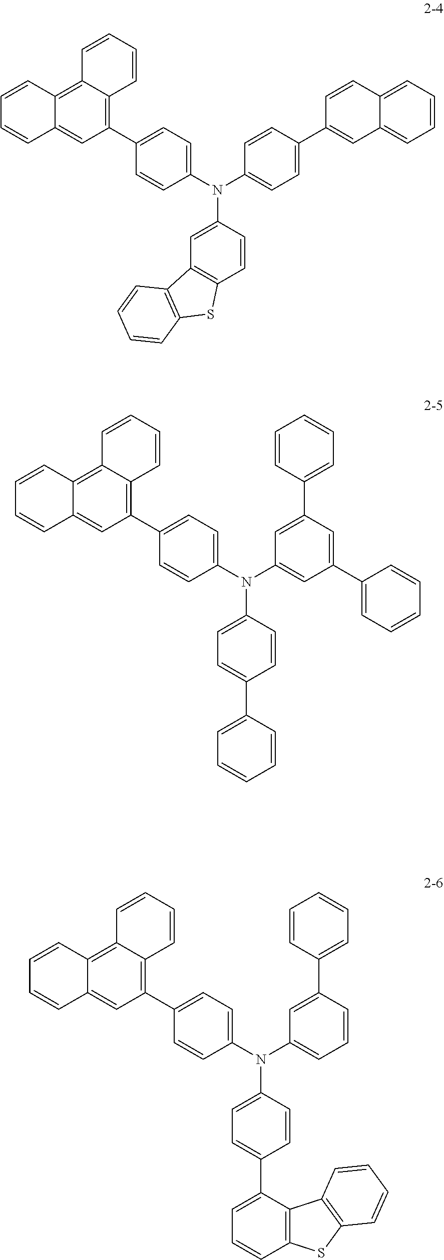 [SMILES: c1ccc2cc(-c3ccc(N(c4ccc(-c5cc6ccccc6c6ccccc56)cc4)c4ccc5sc6ccccc6c5c4)cc3)ccc2c1 ; c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1]

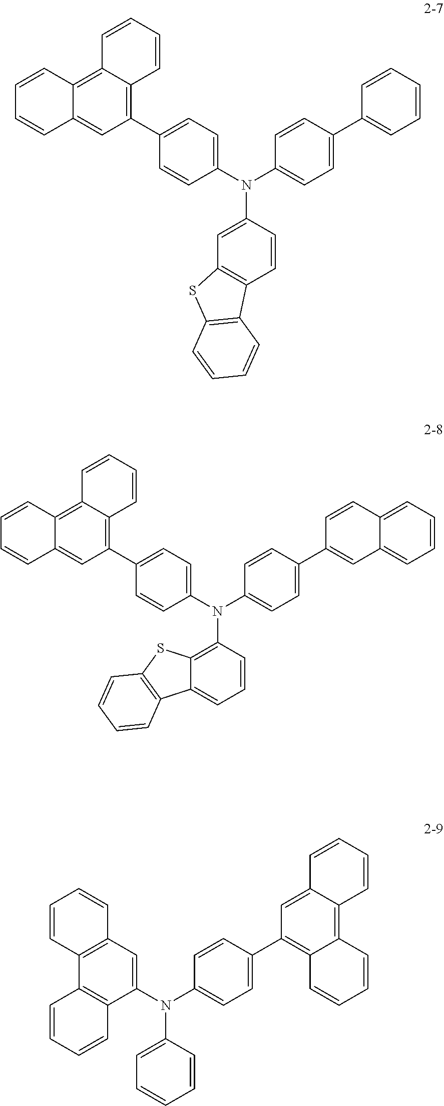 [SMILES: c1ccc(-c2ccc(N(c3ccc(-c4cc5ccccc5c5ccccc45)cc3)c3ccc4c(c3)sc3ccccc34)cc2)cc1 ; c1ccc2cc(-c3ccc(N(c4ccc(-c5cc6ccccc6c6ccccc56)cc4)c4cccc5c4sc4ccccc45)cc3)ccc2c1 ; c1ccc(N(c2ccc(-c3cc4ccccc4c4ccccc34)cc2)c2cc3ccccc3c3ccccc23)cc1]

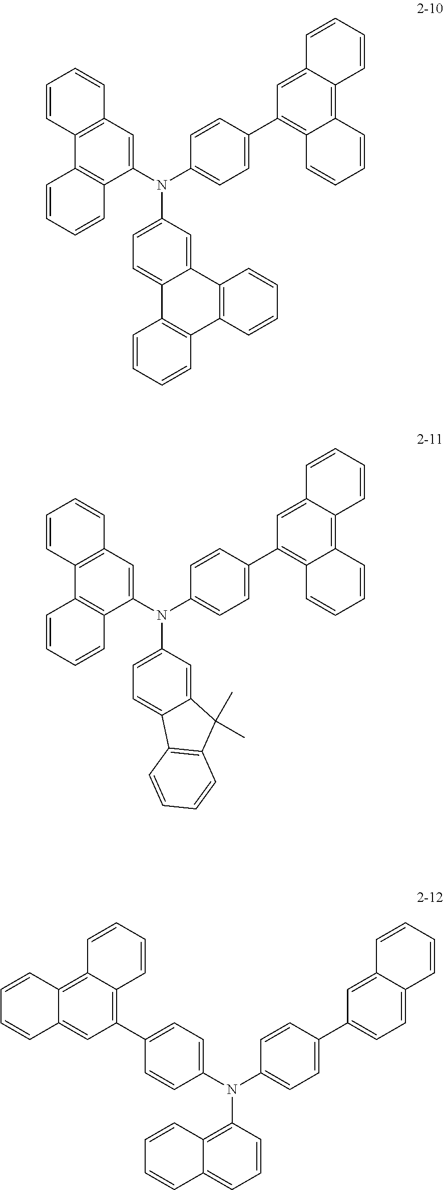 [SMILES: c1ccc2c(c1)cc(-c1ccc(N(c3ccc4c5ccccc5c5ccccc5c4c3)c3cc4ccccc4c4ccccc34)cc1)c1ccccc12 ; CC1(C)c2ccccc2-c2ccc(N(c3ccc(-c4cc5ccccc5c5ccccc45)cc3)c3cc4ccccc4c4ccccc34)cc21 ; c1ccc2cc(-c3ccc(N(c4ccc(-c5cc6ccccc6c6ccccc56)cc4)c4cccc5ccccc45)cc3)ccc2c1]

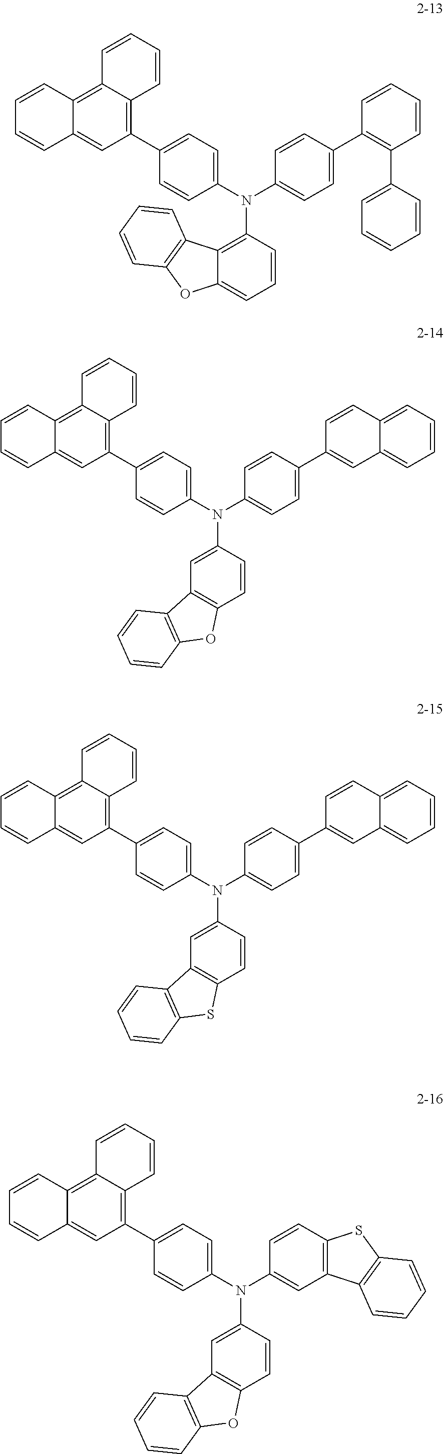 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc2cc(-c3ccc(N(c4ccc(-c5cc6ccccc6c6ccccc56)cc4)c4ccc5oc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1 ; c1ccc2c(c1)cc(-c1ccc(N(c3ccc4oc5ccccc5c4c3)c3ccc4sc5ccccc5c4c3)cc1)c1ccccc12]

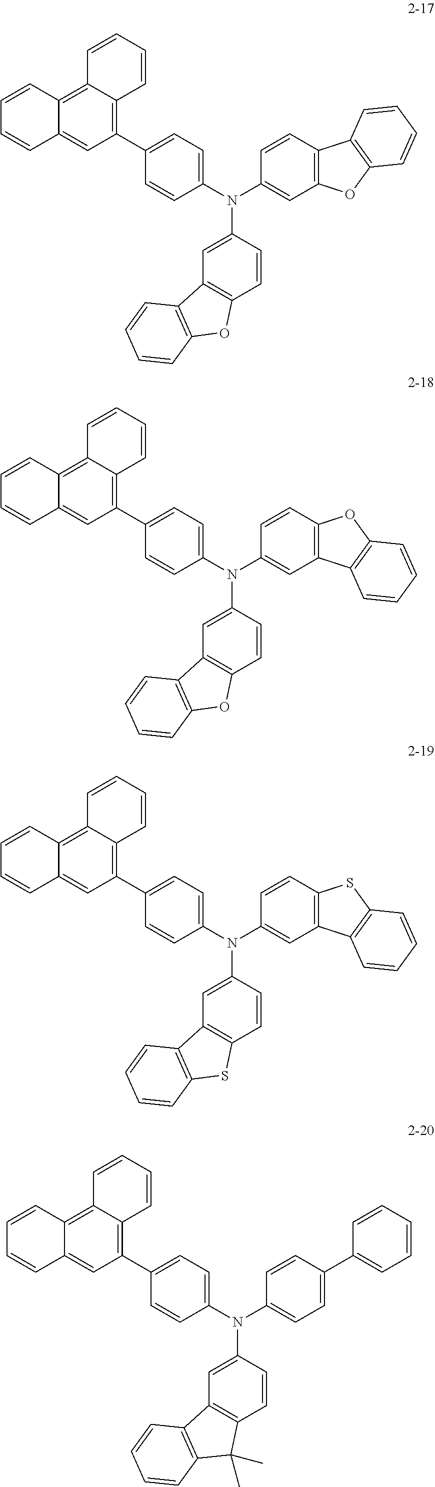 [SMILES: c1ccc2c(c1)cc(-c1ccc(N(c3ccc4c(c3)oc3ccccc34)c3ccc4oc5ccccc5c4c3)cc1)c1ccccc12 ; c1ccc2c(c1)cc(-c1ccc(N(c3ccc4oc5ccccc5c4c3)c3ccc4oc5ccccc5c4c3)cc1)c1ccccc12 ; c1ccc2c(c1)cc(-c1ccc(N(c3ccc4sc5ccccc5c4c3)c3ccc4sc5ccccc5c4c3)cc1)c1ccccc12 ; CC1(C)c2ccccc2-c2cc(N(c3ccc(-c4ccccc4)cc3)c3ccc(-c4cc5ccccc5c5ccccc45)cc3)ccc21]

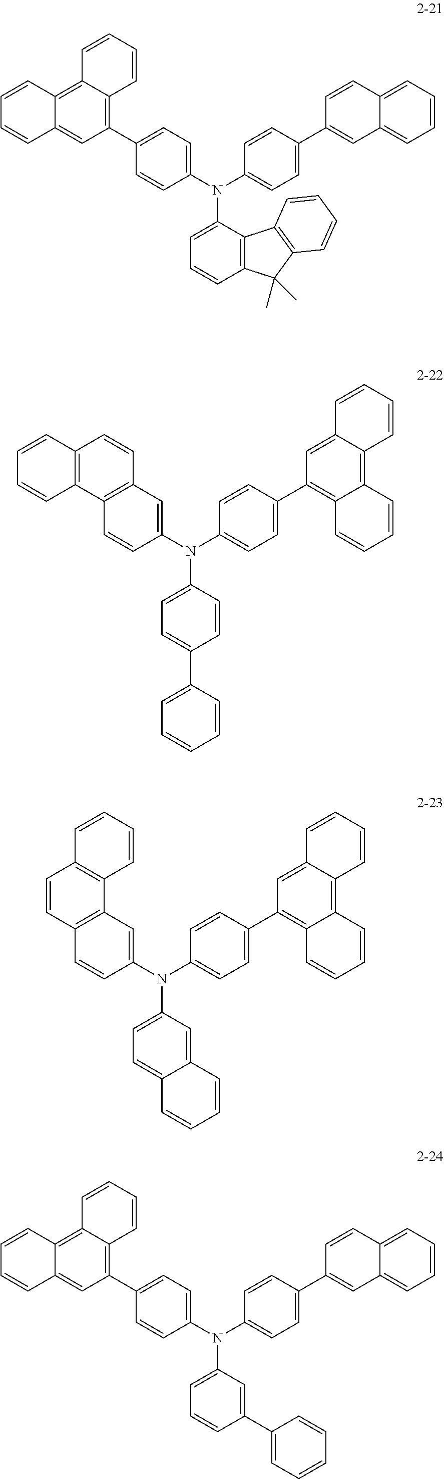 [SMILES: CC1(C)c2ccccc2-c2c(N(c3ccc(-c4ccc5ccccc5c4)cc3)c3ccc(-c4cc5ccccc5c5ccccc45)cc3)cccc21 ; c1ccc(-c2ccc(N(c3ccc(-c4cc5ccccc5c5ccccc45)cc3)c3ccc4c(ccc5ccccc54)c3)cc2)cc1 ; c1ccc2cc(N(c3ccc(-c4cc5ccccc5c5ccccc45)cc3)c3ccc4ccc5ccccc5c4c3)ccc2c1 ; c1ccc(-c2cccc(N(c3ccc(-c4ccc5ccccc5c4)cc3)c3ccc(-c4cc5ccccc5c5ccccc45)cc3)c2)cc1]

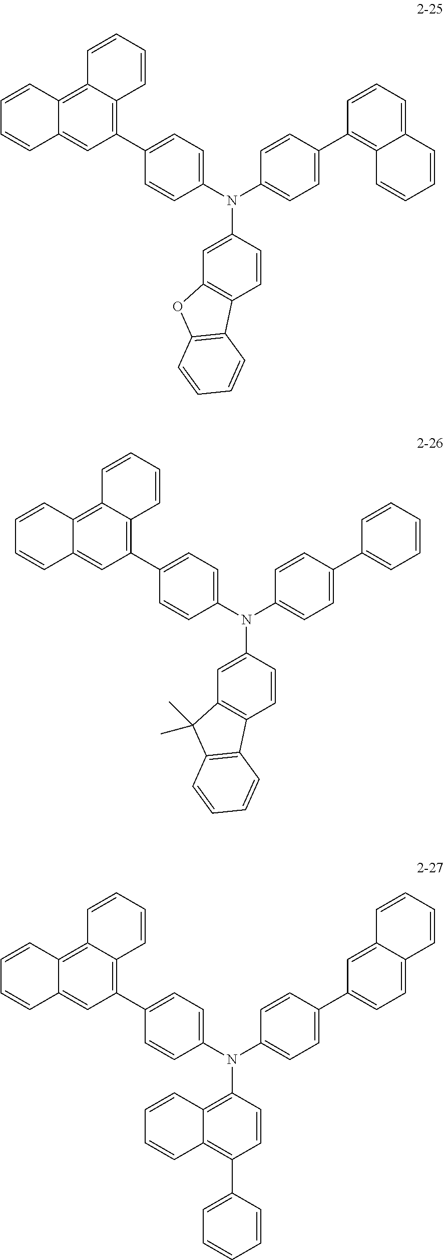 [SMILES: c1ccc2c(-c3ccc(N(c4ccc(-c5cc6ccccc6c6ccccc56)cc4)c4ccc5c(c4)oc4ccccc45)cc3)cccc2c1 ; CC1(C)c2ccccc2-c2ccc(N(c3ccc(-c4ccccc4)cc3)c3ccc(-c4cc5ccccc5c5ccccc45)cc3)cc21 ; c1ccc(-c2ccc(N(c3ccc(-c4ccc5ccccc5c4)cc3)c3ccc(-c4cc5ccccc5c5ccccc45)cc3)c3ccccc23)cc1]

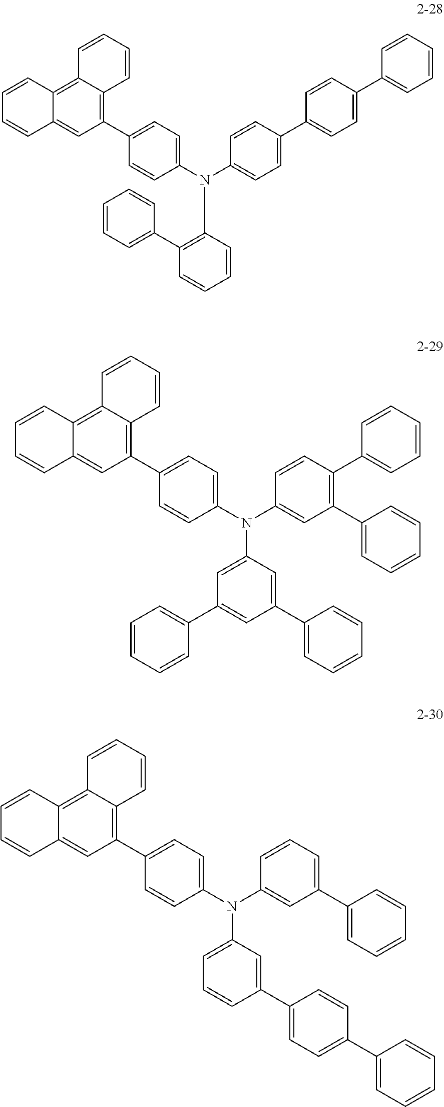 [SMILES: c1ccc(-c2ccc(-c3ccc(N(c4ccc(-c5cc6ccccc6c6ccccc56)cc4)c4ccccc4-c4ccccc4)cc3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2ccc(-c3cccc(N(c4ccc(-c5cc6ccccc6c6ccccc56)cc4)c4cccc(-c5ccccc5)c4)c3)cc2)cc1]

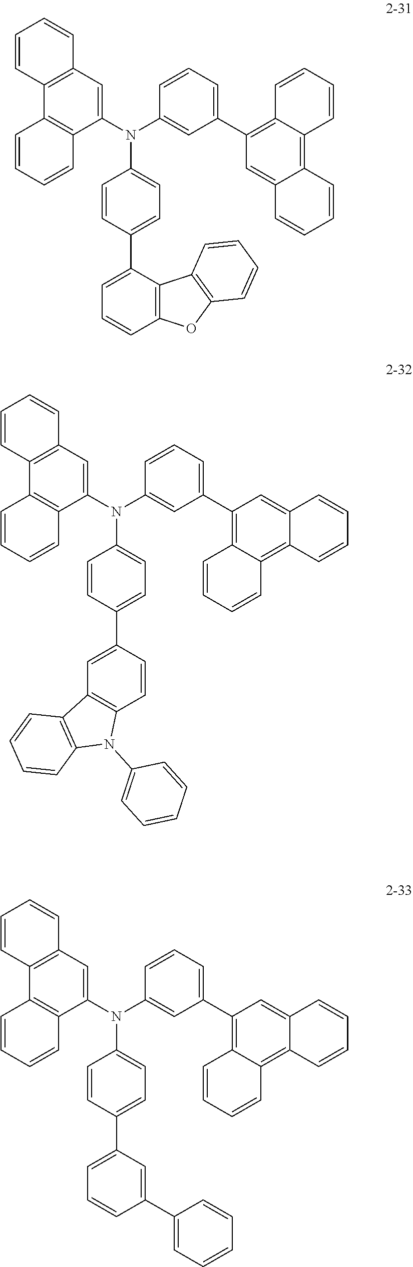 [SMILES: c1cc(-c2cc3ccccc3c3ccccc23)cc(N(c2ccc(-c3cccc4oc5ccccc5c34)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1 ; c1ccc(-c2cccc(-c3ccc(N(c4cccc(-c5cc6ccccc6c6ccccc56)c4)c4cc5ccccc5c5ccccc45)cc3)c2)cc1]

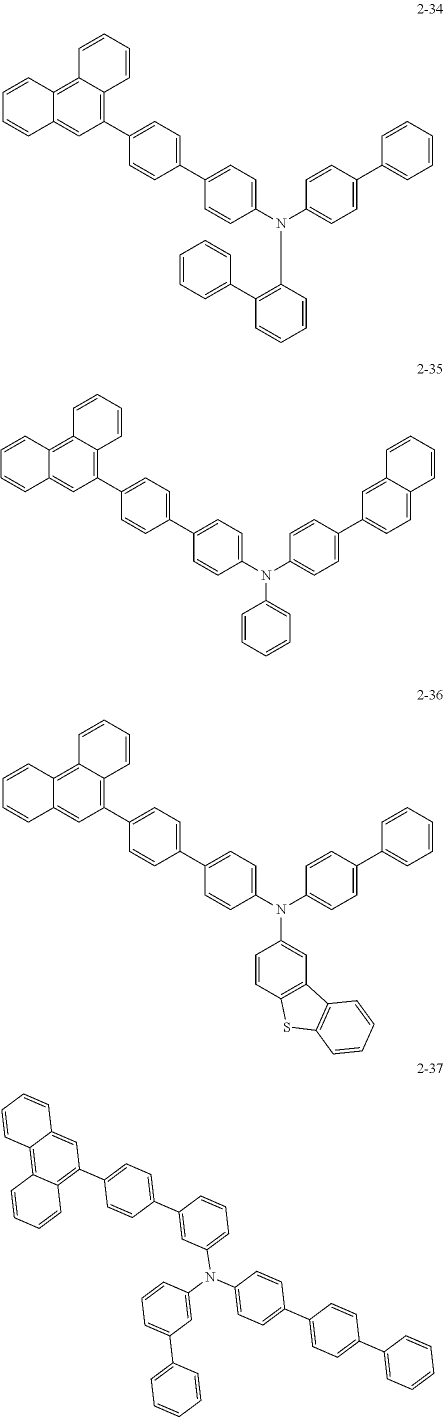 [SMILES: c1ccc(-c2ccc(N(c3ccc(-c4ccc(-c5cc6ccccc6c6ccccc56)cc4)cc3)c3ccccc3-c3ccccc3)cc2)cc1 ; c1ccc(N(c2ccc(-c3ccc(-c4cc5ccccc5c5ccccc45)cc3)cc2)c2ccc(-c3ccc4ccccc4c3)cc2)cc1 ; c1ccc(-c2ccc(N(c3ccc(-c4ccc(-c5cc6ccccc6c6ccccc56)cc4)cc3)c3ccc4sc5ccccc5c4c3)cc2)cc1 ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1]

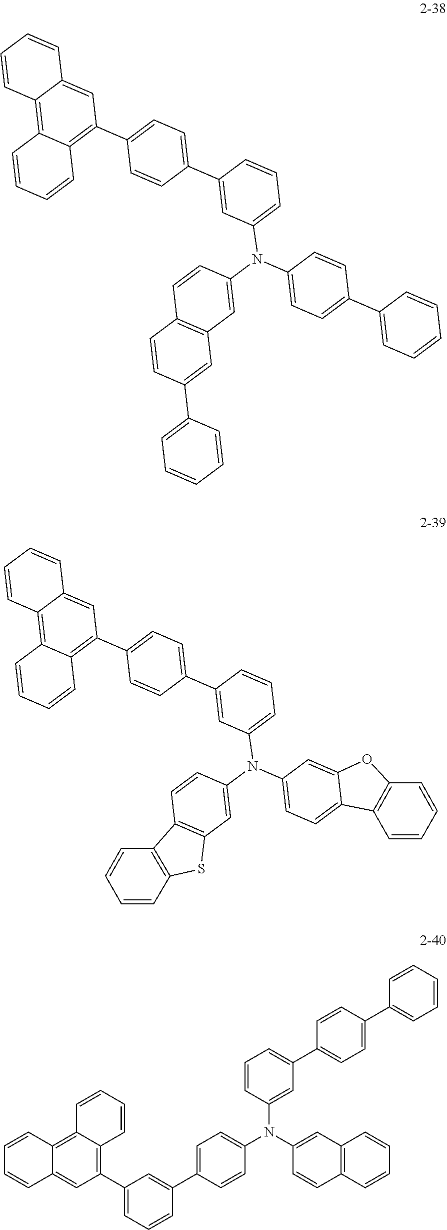 [SMILES: c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1cc(-c2ccc(-c3cc4ccccc4c4ccccc34)cc2)cc(N(c2ccc3c(c2)oc2ccccc23)c2ccc3c(c2)sc2ccccc23)c1 ; c1ccc(-c2ccc(-c3cccc(N(c4ccc(-c5cccc(-c6cc7ccccc7c7ccccc67)c5)cc4)c4ccc5ccccc5c4)c3)cc2)cc1]

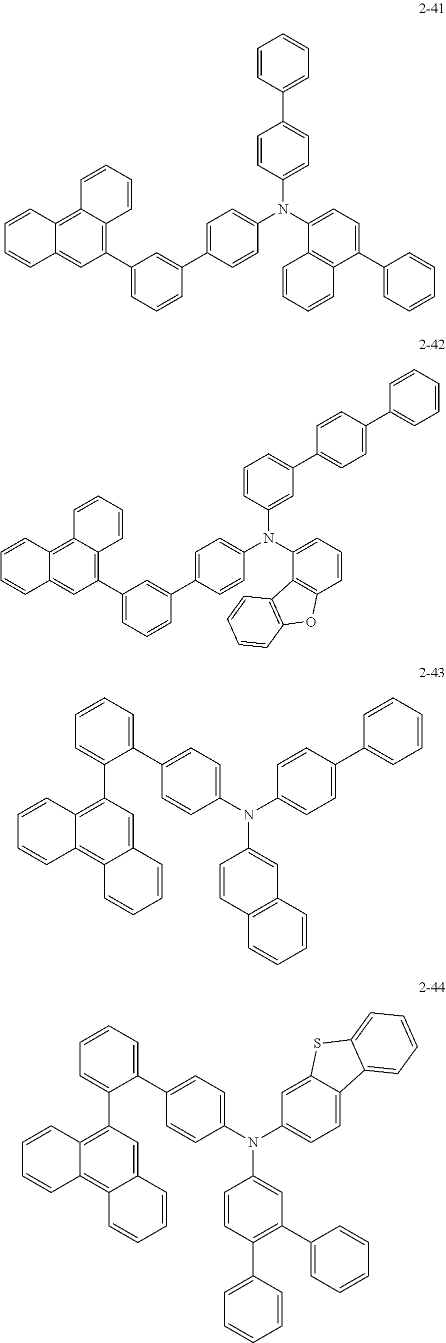 [SMILES: c1ccc(-c2ccc(N(c3ccc(-c4cccc(-c5cc6ccccc6c6ccccc56)c4)cc3)c3ccc(-c4ccccc4)c4ccccc34)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1 ; c1ccc(-c2ccc(N(c3ccc(-c4ccccc4-c4cc5ccccc5c5ccccc45)cc3)c3ccc4ccccc4c3)cc2)cc1 ; c1ccc(-c2ccc(N(c3ccc(-c4ccccc4-c4cc5ccccc5c5ccccc45)cc3)c3ccc4c(c3)sc3ccccc34)cc2-c2ccccc2)cc1]

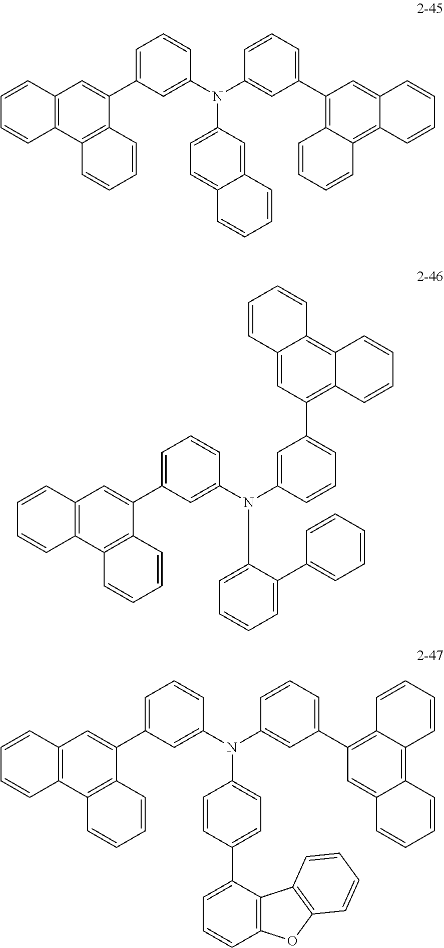 [SMILES: c1cc(-c2cc3ccccc3c3ccccc23)cc(N(c2cccc(-c3cc4ccccc4c4ccccc34)c2)c2ccc3ccccc3c2)c1 ; c1ccc(-c2ccccc2N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1]

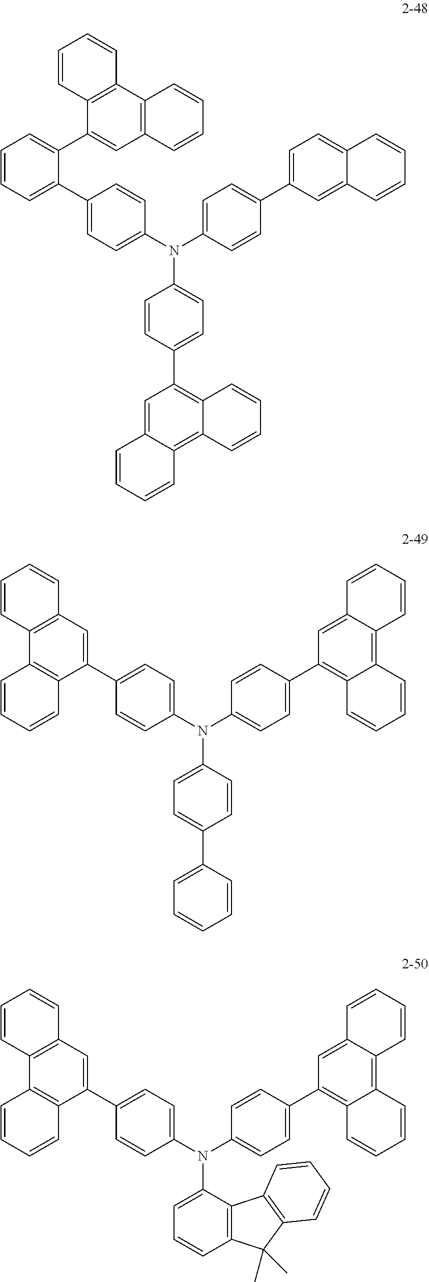 [SMILES: c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1 ; c1ccc(-c2ccc(N(c3ccc(-c4cc5ccccc5c5ccccc45)cc3)c3ccc(-c4cc5ccccc5c5ccccc45)cc3)cc2)cc1 ; CC1(C)c2ccccc2-c2c(N(c3ccc(-c4cc5ccccc5c5ccccc45)cc3)c3ccc(-c4cc5ccccc5c5ccccc45)cc3)cccc21]

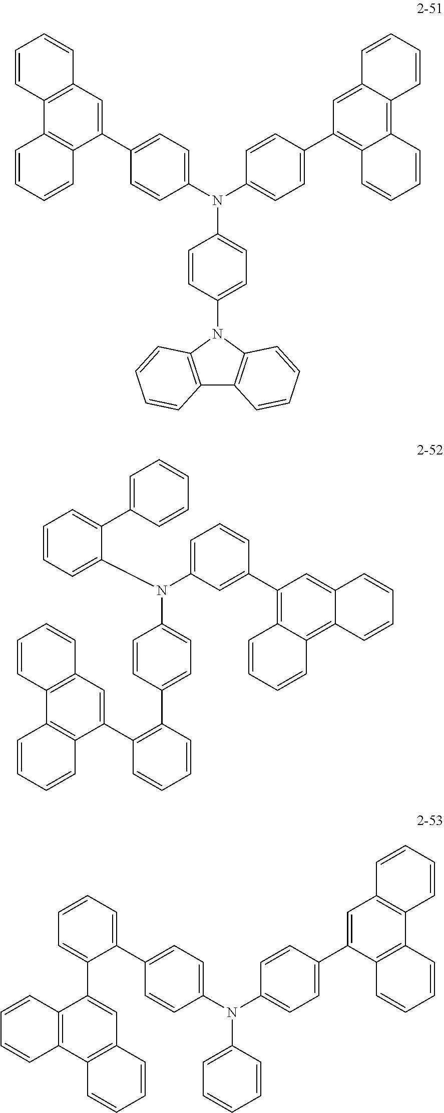 [SMILES: c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12 ; c1ccc(-c2ccccc2N(c2ccc(-c3ccccc3-c3cc4ccccc4c4ccccc34)cc2)c2cccc(-c3cc4ccccc4c4ccccc34)c2)cc1 ; c1ccc(N(c2ccc(-c3ccccc3-c3cc4ccccc4c4ccccc34)cc2)c2ccc(-c3cc4ccccc4c4ccccc34)cc2)cc1]

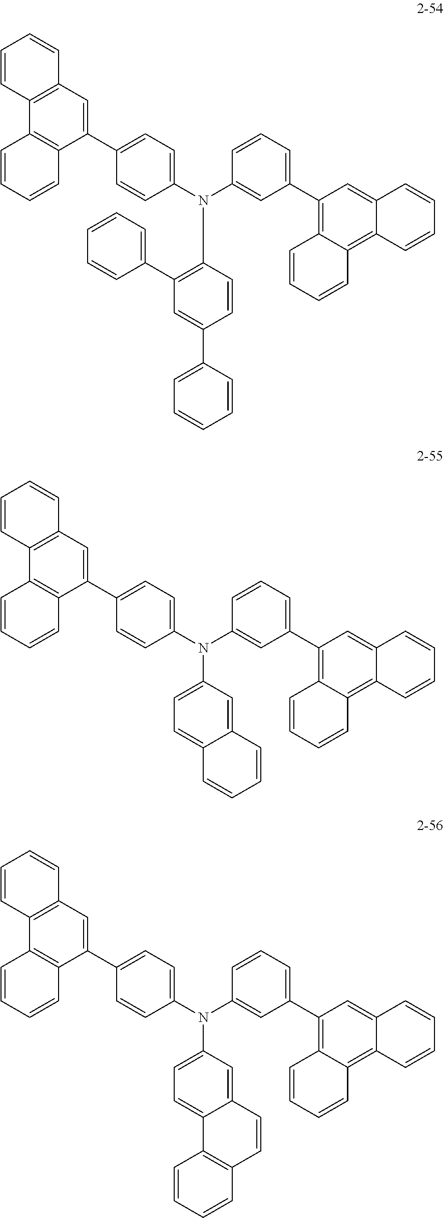 [SMILES: c1ccc(-c2ccc(N(c3ccc(-c4cc5ccccc5c5ccccc45)cc3)c3cccc(-c4cc5ccccc5c5ccccc45)c3)c(-c3ccccc3)c2)cc1 ; c1cc(-c2cc3ccccc3c3ccccc23)cc(N(c2ccc(-c3cc4ccccc4c4ccccc34)cc2)c2ccc3ccccc3c2)c1 ; c1cc(-c2cc3ccccc3c3ccccc23)cc(N(c2ccc(-c3cc4ccccc4c4ccccc34)cc2)c2ccc3c(ccc4ccccc43)c2)c1]

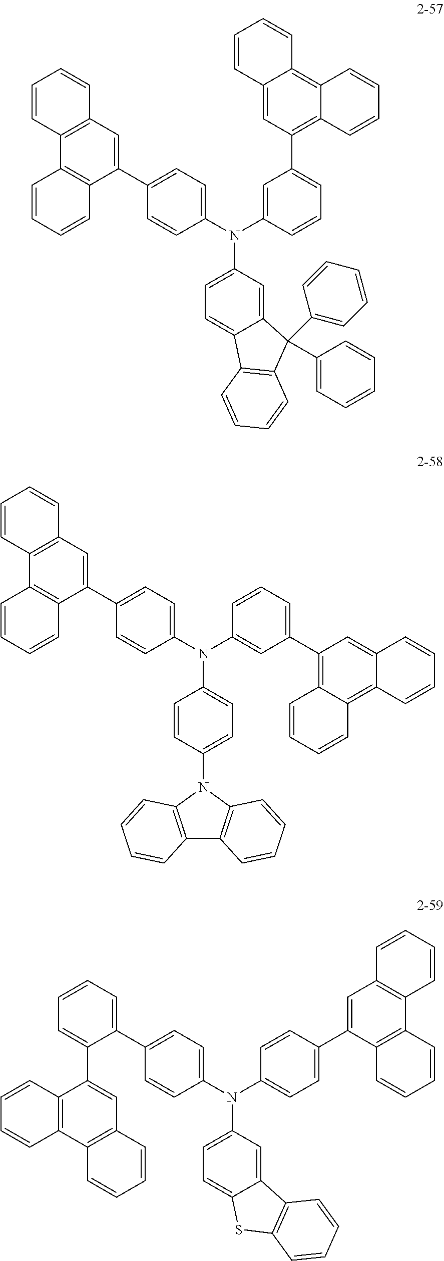 [SMILES: c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1 ; c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1]

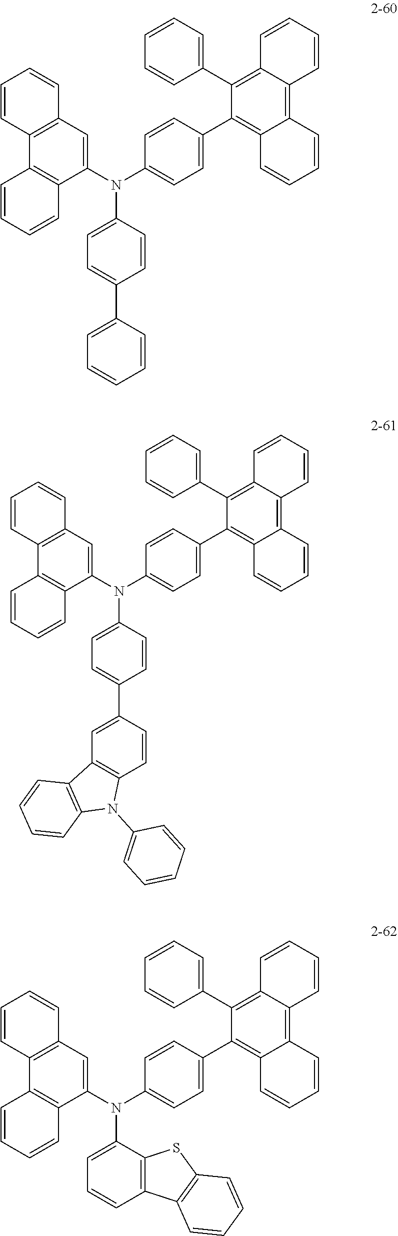 [SMILES: c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3ccc(N(c4cc5ccccc5c5ccccc45)c4cccc5c4sc4ccccc45)cc3)c3ccccc3c3ccccc23)cc1]

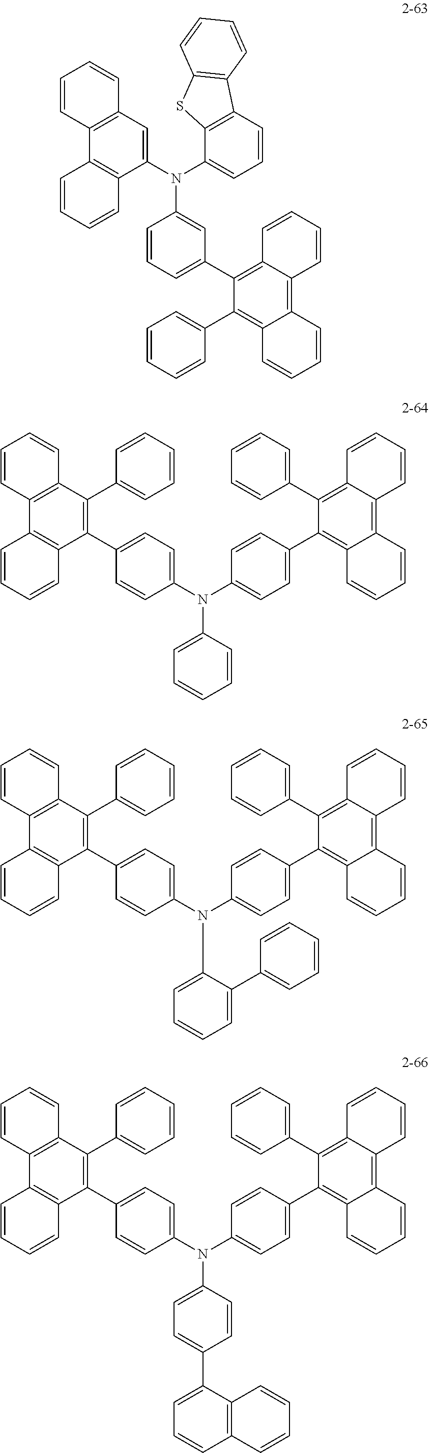 [SMILES: c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1]

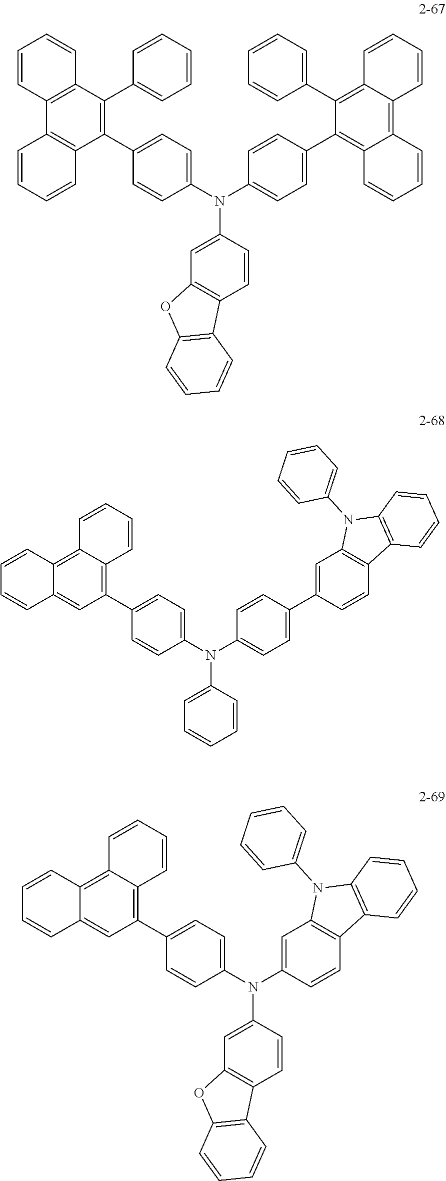 [SMILES: c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1 ; c1ccc(N(c2ccc(-c3ccc4c5ccccc5n(-c5ccccc5)c4c3)cc2)c2ccc(-c3cc4ccccc4c4ccccc34)cc2)cc1 ; c1ccc(-n2c3ccccc3c3ccc(N(c4ccc(-c5cc6ccccc6c6ccccc56)cc4)c4ccc5c(c4)oc4ccccc45)cc32)cc1]

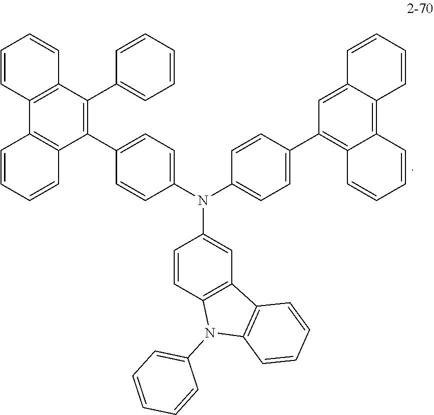 [SMILES: c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1]

### Comparative Examples 1 to 60

An organic light emitting device was manufactured in the same manner as in Example 1, except that the following Compounds B-1 to B-12 as a first host compound and the compound of Chemical Formula 2 as a second host compound were used by co-deposition at a weight ratio of 1:1 as shown in Table 2 below in the organic light emitting device of Example 1.

### Comparative Examples 61 to 156

An organic light emitting device was manufactured in the same manner as in Example 1, except that the compound of Chemical Formula 1 as a first host compound and the following Compounds C-1 to C-12 as a second host compound were used by co-deposition at a weight ratio of 1:1 as shown in Table 3 below in the organic light emitting device of Example 1.

Here, as shown in Tables 2 and 3 below, the structures of Compounds B1 to B12 and Compounds C-1 to C-12 used in Comparative Examples is summarized as follows.

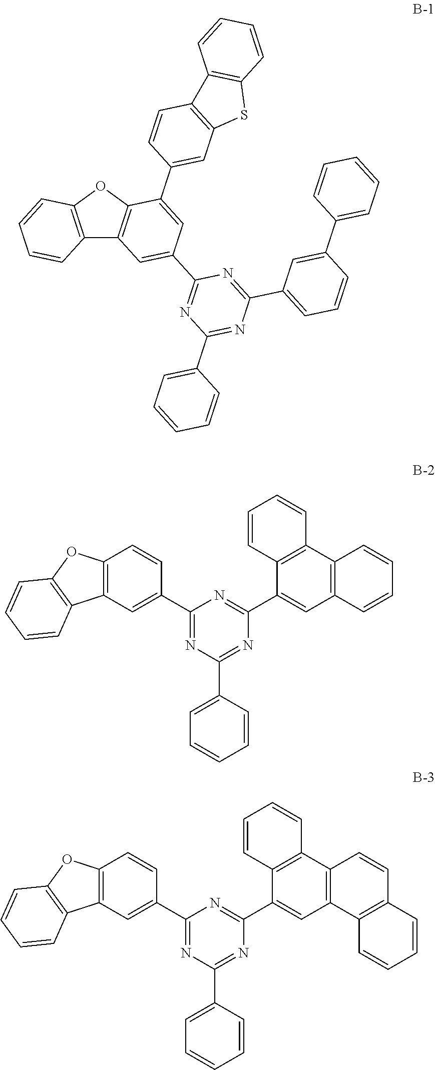 [SMILES: c1ccc(-c2cccc(-c3nc(-c4ccccc4)nc(-c4cc(-c5ccc6c(c5)sc5ccccc56)c5oc6ccccc6c5c4)n3)c2)cc1 ; c1ccc(-c2nc(-c3ccc4oc5ccccc5c4c3)nc(-c3cc4ccccc4c4ccccc34)n2)cc1 ; c1ccc(-c2nc(-c3ccc4oc5ccccc5c4c3)nc(-c3cc4c5ccccc5ccc4c4ccccc34)n2)cc1]

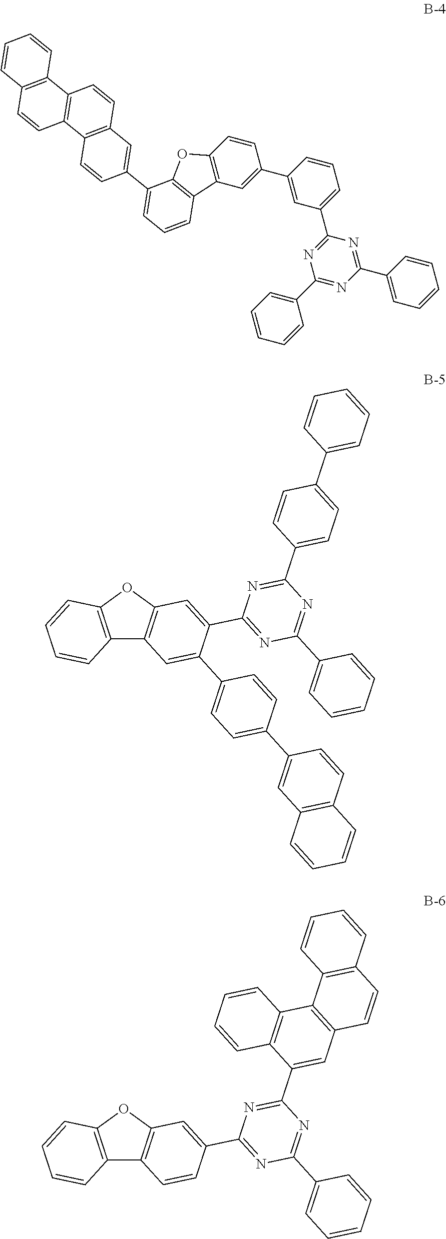 [SMILES: c1ccc(-c2nc(-c3ccccc3)nc(-c3cccc(-c4ccc5oc6c(-c7ccc8c(ccc9c%10ccccc%10ccc89)c7)cccc6c5c4)c3)n2)cc1 ; c1ccc(-c2ccc(-c3nc(-c4ccccc4)nc(-c4cc5oc6ccccc6c5cc4-c4ccc(-c5ccc6ccccc6c5)cc4)n3)cc2)cc1 ; c1ccc(-c2nc(-c3ccc4c(c3)oc3ccccc34)nc(-c3cc4ccc5ccccc5c4c4ccccc34)n2)cc1]

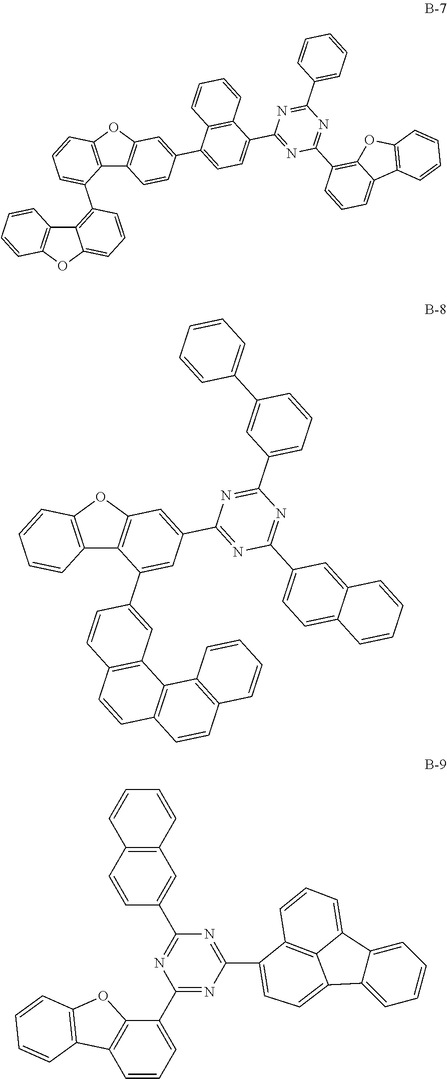 [SMILES: c1ccc(-c2nc(-c3ccc(-c4ccc5c(c4)oc4cccc(-c6cccc7oc8ccccc8c67)c45)c4ccccc34)nc(-c3cccc4c3oc3ccccc34)n2)cc1 ; c1ccc(-c2cccc(-c3nc(-c4ccc5ccccc5c4)nc(-c4cc(-c5ccc6ccc7ccc8ccccc8c7c6c5)c5c(c4)oc4ccccc45)n3)c2)cc1 ; c1ccc2c(c1)-c1cccc3c(-c4nc(-c5ccc6ccccc6c5)nc(-c5cccc6c5oc5ccccc56)n4)ccc-2c13]

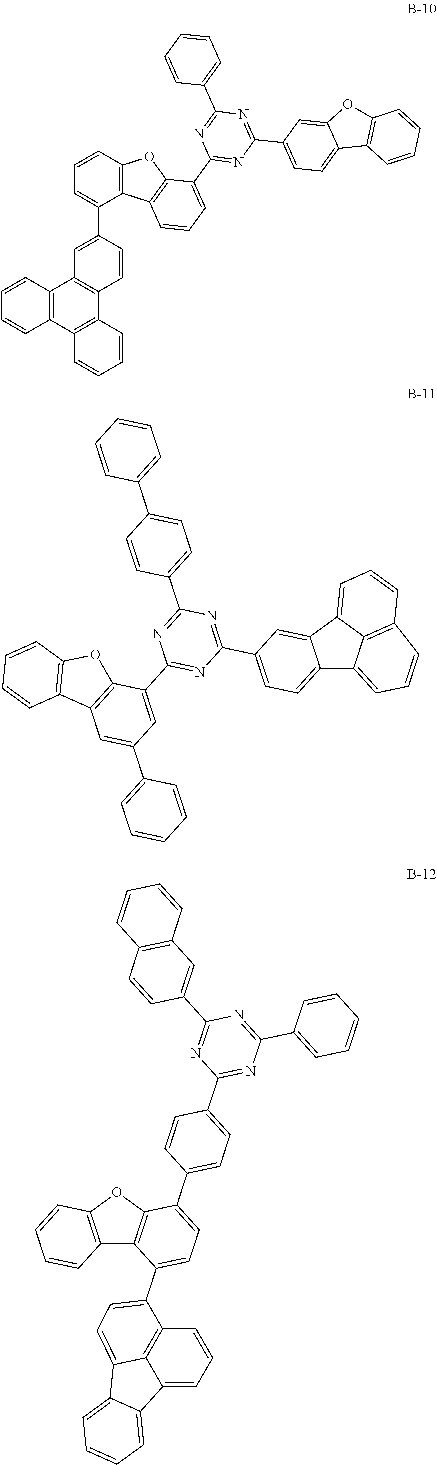

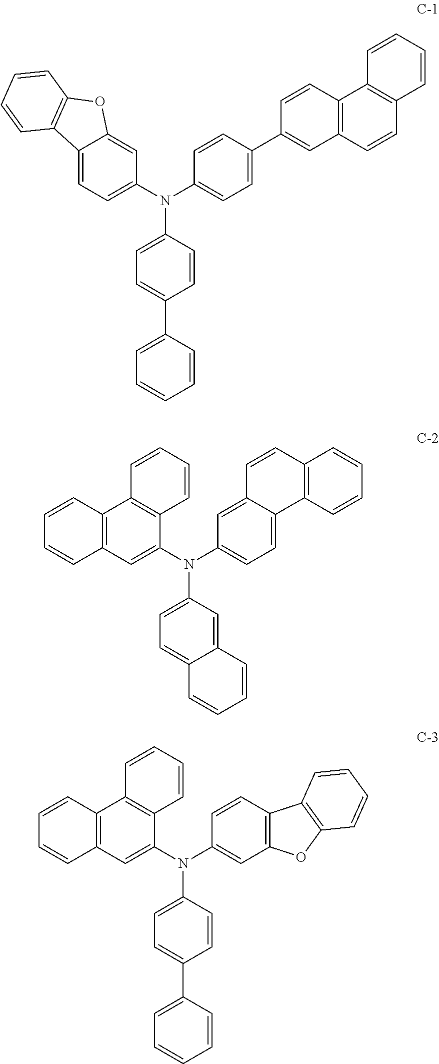 [SMILES: c1ccc(-c2ccc(N(c3ccc(-c4ccc5c(ccc6ccccc65)c4)cc3)c3ccc4c(c3)oc3ccccc34)cc2)cc1 ; c1ccc2cc(N(c3ccc4c(ccc5ccccc54)c3)c3cc4ccccc4c4ccccc34)ccc2c1 ; c1ccc(-c2ccc(N(c3ccc4c(c3)oc3ccccc34)c3cc4ccccc4c4ccccc34)cc2)cc1]

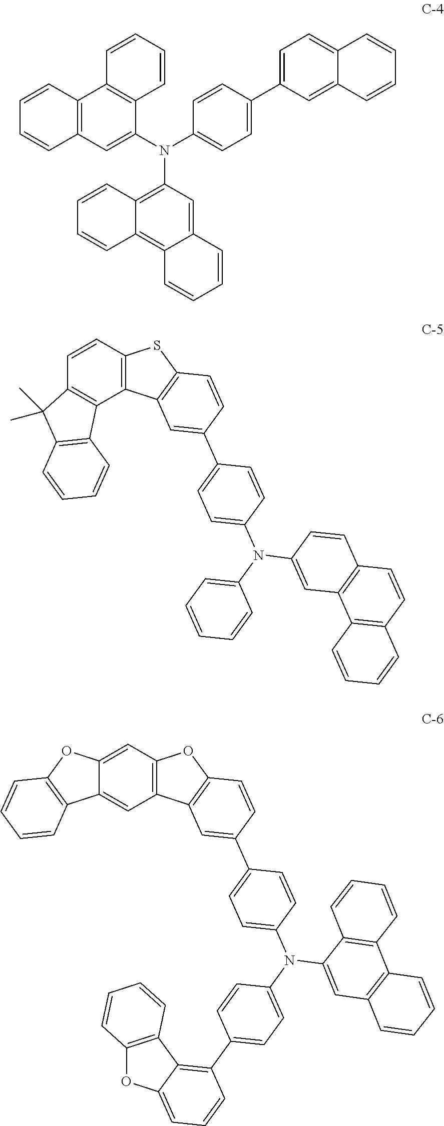 [SMILES: c1ccc2cc(-c3ccc(N(c4cc5ccccc5c5ccccc45)c4cc5ccccc5c5ccccc45)cc3)ccc2c1 ; CC1(C)c2ccccc2-c2c1ccc1sc3ccc(-c4ccc(N(c5ccccc5)c5ccc6ccc7ccccc7c6c5)cc4)cc3c21 ; c1ccc2c(c1)cc(N(c1ccc(-c3ccc4oc5cc6oc7ccccc7c6cc5c4c3)cc1)c1ccc(-c3cccc4oc5ccccc5c34)cc1)c1ccccc12]

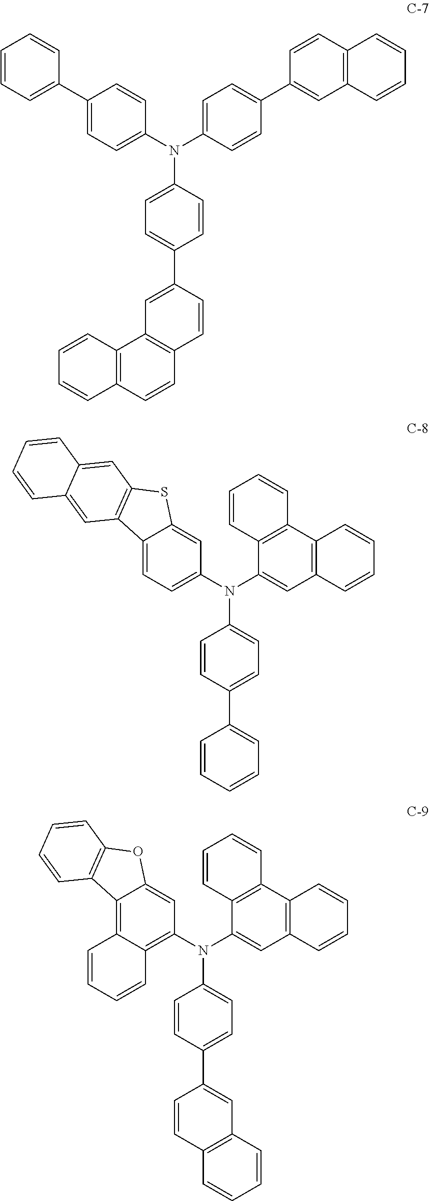

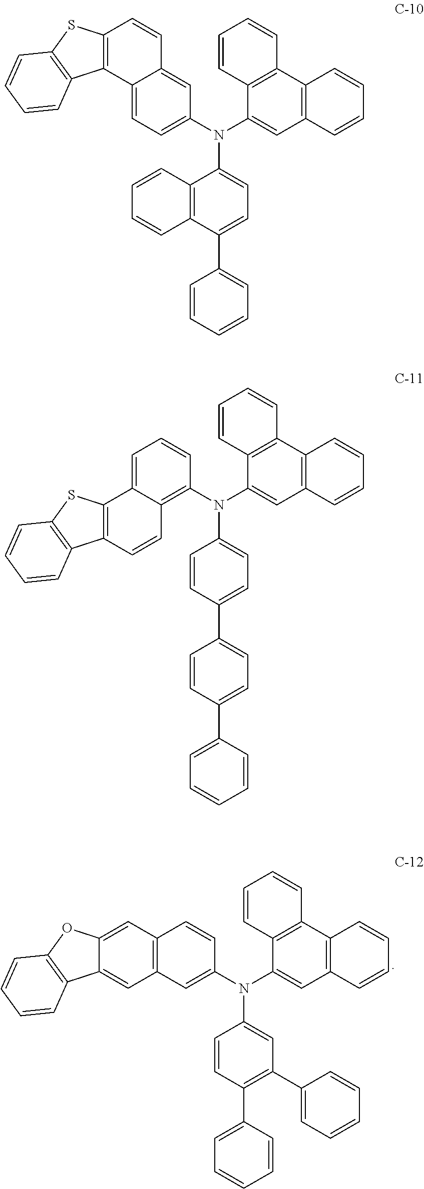 [SMILES: c1ccc(-c2ccc(N(c3ccc4c(ccc5sc6ccccc6c54)c3)c3cc4ccccc4c4ccccc34)c3ccccc23)cc1 ; c1ccc(-c2ccc(-c3ccc(N(c4cc5ccccc5c5ccccc45)c4cccc5c4ccc4c6ccccc6sc54)cc3)cc2)cc1 ; c1ccc(-c2ccc(N(c3ccc4cc5oc6ccccc6c5cc4c3)c3cc4ccccc4c4ccccc34)cc2-c2ccccc2)cc1]

### Experimental Examples

For the organic light emitting devices prepared in Examples and Comparative Examples, the voltage, efficiency, and lifespan (T95) were measured by applying a current, and the results are shown in Tables 1 to 3 below. At this time, the voltage and efficiency were measured by applying a current density of 15 mA/cm². In addition, T95 of Tables 1 to 3 below means the time (hr) taken until the initial luminance (6,000 nit) decreases to 95%.

TABLE 1

| | | | Voltage | Efficiency | Lifespan | Emission |
|---|---|---|---|---|---|---|
| Category | First host | Second host | (V) | (cd/A) | T95(hr) | color |
| Example 1 | Compound 1-1 | Compound 2-1 | 3.41 | 24.26 | 306 | Red |
| Example 2 | Compound 1-1 | Compound 2-31 | 3.48 | 24.55 | 314 | Red |
| Example 3 | Compound 1-1 | Compound 2-40 | 3.40 | 24.30 | 313 | Red |
| Example 4 | Compound 1-1 | Compound 2-45 | 3.47 | 24.06 | 298 | Red |
| Example 5 | Compound 1-1 | Compound 2-60 | 3.41 | 24.25 | 312 | Red |
| Example 6 | Compound 1-2 | Compound 2-2 | 3.45 | 21.90 | 304 | Red |
| Example 7 | Compound 1-2 | Compound 2-32 | 3.46 | 22.51 | 300 | Red |
| Example 8 | Compound 1-2 | Compound 2-23 | 3.44 | 21.44 | 310 | Red |
| Example 9 | Compound 1-2 | Compound 2-46 | 3.40 | 23.17 | 291 | Red |
| Example 10 | Compound 1-2 | Compound 2-61 | 3.38 | 21.43 | 320 | Red |
| Example 11 | Compound 1-3 | Compound 2-3 | 3.36 | 23.22 | 315 | Red |
| Example 12 | Compound 1-3 | Compound 2-33 | 3.42 | 22.31 | 316 | Red |
| Example 13 | Compound 1-3 | Compound 2-24 | 3.36 | 22.96 | 300 | Red |
| Example 14 | Compound 1-3 | Compound 2-47 | 3.49 | 23.22 | 293 | Red |
| Example 15 | Compound 1-3 | Compound 2-62 | 3.49 | 22.59 | 298 | Red |
| Example 16 | Compound 1-4 | Compound 2-4 | 3.35 | 24.40 | 291 | Red |
| Example 17 | Compound 1-4 | Compound 2-16 | 3.33 | 24.04 | 292 | Red |
| Example 18 | Compound 1-4 | Compound 2-43 | 3.35 | 23.18 | 303 | Red |
| Example 19 | Compound 1-4 | Compound 2-48 | 3.46 | 23.73 | 320 | Red |
| Example 20 | Compound 1-4 | Compound 2-63 | 3.40 | 24.62 | 316 | Red |
| Example 21 | Compound 1-5 | Compound 2-5 | 3.43 | 22.86 | 292 | Red |
| Example 22 | Compound 1-5 | Compound 2-17 | 3.45 | 22.04 | 306 | Red |
| Example 23 | Compound 1-5 | Compound 2-44 | 3.40 | 22.14 | 311 | Red |
| Example 24 | Compound 1-5 | Compound 2-49 | 3.32 | 21.56 | 313 | Red |
| Example 25 | Compound 1-5 | Compound 2-64 | 3.43 | 23.20 | 302 | Red |
| Example 26 | Compound 1-6 | Compound 2-6 | 3.41 | 23.29 | 307 | Red |
| Example 27 | Compound 1-6 | Compound 2-18 | 3.40 | 23.62 | 319 | Red |
| Example 28 | Compound 1-6 | Compound 2-41 | 3.39 | 24.92 | 308 | Red |
| Example 29 | Compound 1-6 | Compound 2-50 | 3.40 | 23.85 | 308 | Red |
| Example 30 | Compound 1-6 | Compound 2-65 | 3.36 | 24.10 | 310 | Red |
| Example 31 | Compound 1-7 | Compound 2-7 | 3.58 | 22.69 | 279 | Red |
| Example 32 | Compound 1-7 | Compound 2-19 | 3.53 | 22.78 | 273 | Red |
| Example 33 | Compound 1-7 | Compound 2-42 | 3.48 | 21.34 | 266 | Red |
| Example 34 | Compound 1-7 | Compound 2-51 | 3.47 | 22.79 | 275 | Red |
| Example 35 | Compound 1-7 | Compound 2-66 | 3.61 | 22.02 | 278 | Red |
| Example 36 | Compound 1-8 | Compound 2-8 | 3.55 | 23.00 | 272 | Red |
| Example 37 | Compound 1-8 | Compound 2-34 | 3.53 | 22.98 | 273 | Red |
| Example 38 | Compound 1-8 | Compound 2-25 | 3.61 | 21.86 | 291 | Red |
| Example 39 | Compound 1-8 | Compound 2-52 | 3.55 | 22.59 | 264 | Red |
| Example 40 | Compound 1-8 | Compound 2-67 | 3.59 | 21.94 | 277 | Red |
| Example 41 | Compound 1-9 | Compound 2-9 | 3.41 | 22.54 | 304 | Red |
| Example 42 | Compound 1-9 | Compound 2-35 | 3.49 | 21.33 | 319 | Red |
| Example 43 | Compound 1-9 | Compound 2-26 | 3.48 | 21.22 | 316 | Red |
| Example 44 | Compound 1-9 | Compound 2-53 | 3.33 | 21.49 | 314 | Red |
| Example 45 | Compound 1-9 | Compound 2-68 | 3.38 | 21.32 | 315 | Red |
| Example 46 | Compound 1-10 | Compound 2-10 | 3.46 | 21.19 | 300 | Red |
| Example 47 | Compound 1-10 | Compound 2-36 | 3.32 | 22.98 | 302 | Red |
| Example 48 | Compound 1-10 | Compound 2-27 | 3.36 | 21.85 | 303 | Red |
| Example 49 | Compound 1-10 | Compound 2-54 | 3.49 | 21.64 | 319 | Red |
| Example 50 | Compound 1-10 | Compound 2-69 | 3.38 | 21.24 | 291 | Red |
| Example 51 | Compound 1-11 | Compound 2-11 | 3.56 | 21.74 | 260 | Red |
| Example 52 | Compound 1-11 | Compound 2-37 | 3.51 | 21.45 | 262 | Red |
| Example 53 | Compound 1-11 | Compound 2-28 | 3.47 | 21.87 | 263 | Red |
| Example 54 | Compound 1-11 | Compound 2-55 | 3.57 | 23.08 | 288 | Red |
| Example 55 | Compound 1-11 | Compound 2-70 | 3.57 | 22.54 | 293 | Red |
| Example 56 | Compound 1-12 | Compound 2-12 | 3.39 | 23.52 | 293 | Red |
| Example 57 | Compound 1-12 | Compound 2-20 | 3.35 | 23.16 | 319 | Red |
| Example 58 | Compound 1-12 | Compound 2-43 | 3.34 | 24.64 | 304 | Red |
| Example 59 | Compound 1-12 | Compound 2-56 | 3.45 | 24.79 | 316 | Red |
| Example 60 | Compound 1-12 | Compound 2-60 | 3.46 | 25.06 | 293 | Red |
| Example 61 | Compound 1-13 | Compound 2-13 | 3.44 | 24.35 | 319 | Red |
| Example 62 | Compound 1-13 | Compound 2-21 | 3.45 | 23.20 | 290 | Red |
| Example 63 | Compound 1-13 | Compound 2-44 | 3.39 | 23.52 | 294 | Red |
| Example 64 | Compound 1-13 | Compound 2-57 | 3.46 | 23.53 | 296 | Red |
| Example 65 | Compound 1-13 | Compound 2-62 | 3.32 | 23.82 | 303 | Red |
| Example 66 | Compound 1-14 | Compound 2-19 | 3.39 | 21.74 | 293 | Red |
| Example 67 | Compound 1-14 | Compound 2-38 | 3.35 | 21.45 | 319 | Red |
| Example 68 | Compound 1-14 | Compound 2-29 | 3.34 | 21.87 | 304 | Red |
| Example 69 | Compound 1-14 | Compound 2-58 | 3.45 | 23.08 | 316 | Red |
| Example 70 | Compound 1-14 | Compound 2-64 | 3.46 | 22.54 | 293 | Red |
| Example 71 | Compound 1-15 | Compound 2-14 | 3.44 | 22.62 | 319 | Red |
| Example 72 | Compound 1-15 | Compound 2-22 | 3.45 | 21.26 | 290 | Red |
| Example 73 | Compound 1-15 | Compound 2-40 | 3.39 | 22.78 | 294 | Red |
| Example 74 | Compound 1-15 | Compound 2-59 | 3.46 | 22.44 | 296 | Red |
| Example 75 | Compound 1-15 | Compound 2-65 | 3.32 | 21.80 | 303 | Red |
| Example 76 | Compound 1-16 | Compound 2-15 | 3.47 | 21.51 | 279 | Red |
| Example 77 | Compound 1-16 | Compound 2-39 | 3.56 | 22.78 | 268 | Red |
| Example 78 | Compound 1-16 | Compound 2-30 | 3.48 | 22.03 | 293 | Red |
| Example 79 | Compound 1-16 | Compound 2-47 | 3.55 | 21.38 | 273 | Red |
| Example 80 | Compound 1-16 | Compound 2-67 | 3.50 | 22.12 | 275 | Red |
| Example 81 | Compound 1-17 | Compound 2-1 | 3.36 | 23.13 | 299 | Red |
| Example 82 | Compound 1-17 | Compound 2-31 | 3.48 | 23.14 | 296 | Red |
| Example 83 | Compound 1-17 | Compound 2-40 | 3.40 | 23.34 | 306 | Red |
| Example 84 | Compound 1-17 | Compound 2-45 | 3.34 | 23.58 | 294 | Red |
| Example 85 | Compound 1-17 | Compound 2-60 | 3.34 | 23.29 | 317 | Red |
| Example 86 | Compound 1-18 | Compound 2-2 | 3.37 | 21.51 | 318 | Red |
| Example 87 | Compound 1-18 | Compound 2-32 | 3.44 | 22.78 | 315 | Red |
| Example 88 | Compound 1-18 | Compound 2-23 | 3.41 | 22.03 | 301 | Red |
| Example 89 | Compound 1-18 | Compound 2-46 | 3.45 | 21.38 | 290 | Red |
| Example 90 | Compound 1-18 | Compound 2-61 | 3.44 | 22.12 | 306 | Red |
| Example 91 | Compound 1-19 | Compound 2-3 | 3.53 | 22.22 | 262 | Red |
| Example 92 | Compound 1-19 | Compound 2-33 | 3.49 | 22.27 | 277 | Red |
| Example 93 | Compound 1-19 | Compound 2-24 | 3.54 | 21.38 | 292 | Red |
| Example 94 | Compound 1-19 | Compound 2-47 | 3.48 | 21.63 | 279 | Red |
| Example 95 | Compound 1-19 | Compound 2-62 | 3.51 | 22.31 | 287 | Red |
| Example 96 | Compound 1-20 | Compound 2-4 | 3.54 | 22.15 | 283 | Red |
| Example 97 | Compound 1-20 | Compound 2-16 | 3.56 | 23.19 | 263 | Red |
| Example 98 | Compound 1-20 | Compound 2-43 | 3.61 | 21.96 | 265 | Red |
| Example 99 | Compound 1-20 | Compound 2-48 | 3.50 | 22.86 | 279 | Red |
| Example 100 | Compound 1-20 | Compound 2-63 | 3.47 | 21.96 | 278 | Red |
| Example 101 | Compound 1-21 | Compound 2-1 | 3.34 | 21.43 | 293 | Red |
| Example 102 | Compound 1-21 | Compound 2-31 | 3.36 | 21.57 | 314 | Red |
| Example 103 | Compound 1-21 | Compound 2-40 | 3.49 | 21.14 | 300 | Red |
| Example 104 | Compound 1-21 | Compound 2-45 | 3.38 | 21.20 | 299 | Red |
| Example 105 | Compound 1-21 | Compound 2-60 | 3.39 | 22.80 | 301 | Red |
| Example 106 | Compound 1-22 | Compound 2-2 | 3.47 | 22.00 | 313 | Red |
| Example 107 | Compound 1-22 | Compound 2-32 | 3.38 | 21.20 | 297 | Red |
| Example 108 | Compound 1-22 | Compound 2-23 | 3.38 | 22.52 | 305 | Red |
| Example 109 | Compound 1-22 | Compound 2-46 | 3.43 | 23.05 | 318 | Red |
| Example 110 | Compound 1-22 | Compound 2-61 | 3.41 | 21.26 | 290 | Red |
| Example 111 | Compound 1-23 | Compound 2-3 | 3.49 | 23.82 | 305 | Red |
| Example 112 | Compound 1-23 | Compound 2-33 | 3.37 | 23.59 | 296 | Red |
| Example 113 | Compound 1-23 | Compound 2-24 | 3.35 | 24.19 | 311 | Red |
| Example 114 | Compound 1-23 | Compound 2-47 | 3.46 | 23.67 | 297 | Red |
| Example 115 | Compound 1-23 | Compound 2-62 | 3.36 | 24.14 | 298 | Red |
| Example 116 | Compound 1-24 | Compound 2-4 | 3.39 | 24.91 | 307 | Red |
| Example 117 | Compound 1-24 | Compound 2-16 | 3.39 | 24.14 | 307 | Red |
| Example 118 | Compound 1-24 | Compound 2-43 | 3.44 | 24.65 | 290 | Red |
| Example 119 | Compound 1-24 | Compound 2-48 | 3.34 | 23.65 | 304 | Red |
| Example 120 | Compound 1-24 | Compound 2-63 | 3.45 | 24.38 | 305 | Red |
| Example 121 | Compound 1-25 | Compound 2-5 | 3.60 | 22.22 | 278 | Red |
| Example 122 | Compound 1-25 | Compound 2-17 | 3.61 | 21.17 | 271 | Red |
| Example 123 | Compound 1-25 | Compound 2-44 | 3.48 | 22.35 | 275 | Red |
| Example 124 | Compound 1-25 | Compound 2-49 | 3.48 | 21.69 | 290 | Red |
| Example 125 | Compound 1-25 | Compound 2-64 | 3.57 | 21.80 | 260 | Red |
| Example 126 | Compound 1-26 | Compound 2-6 | 3.51 | 22.15 | 273 | Red |
| Example 127 | Compound 1-26 | Compound 2-18 | 3.52 | 21.65 | 274 | Red |
| Example 128 | Compound 1-26 | Compound 2-41 | 3.53 | 23.07 | 278 | Red |
| Example 129 | Compound 1-26 | Compound 2-50 | 3.61 | 22.56 | 266 | Red |
| Example 130 | Compound 1-26 | Compound 2-65 | 3.51 | 21.63 | 280 | Red |
| Example 131 | Compound 1-27 | Compound 2-7 | 3.37 | 23.26 | 297 | Red |
| Example 132 | Compound 1-27 | Compound 2-19 | 3.37 | 21.30 | 309 | Red |
| Example 133 | Compound 1-27 | Compound 2-42 | 3.47 | 22.13 | 306 | Red |
| Example 134 | Compound 1-27 | Compound 2-51 | 3.41 | 21.90 | 306 | Red |
| Example 135 | Compound 1-27 | Compound 2-66 | 3.46 | 21.59 | 298 | Red |
| Example 136 | Compound 1-28 | Compound 2-8 | 3.37 | 23.96 | 297 | Red |
| Example 137 | Compound 1-28 | Compound 2-34 | 3.37 | 24.49 | 309 | Red |
| Example 138 | Compound 1-28 | Compound 2-25 | 3.47 | 23.15 | 306 | Red |
| Example 139 | Compound 1-28 | Compound 2-52 | 3.41 | 25.02 | 306 | Red |
| Example 140 | Compound 1-28 | Compound 2-67 | 3.46 | 23.69 | 298 | Red |
| Example 141 | Compound 1-29 | Compound 2-9 | 3.50 | 22.96 | 277 | Red |
| Example 142 | Compound 1-29 | Compound 2-35 | 3.56 | 21.17 | 293 | Red |
| Example 143 | Compound 1-29 | Compound 2-26 | 3.50 | 22.00 | 270 | Red |
| Example 144 | Compound 1-29 | Compound 2-53 | 3.48 | 21.26 | 282 | Red |
| Example 145 | Compound 1-29 | Compound 2-68 | 3.61 | 22.61 | 276 | Red |
| Example 146 | Compound 1-30 | Compound 2-10 | 3.49 | 21.99 | 275 | Red |
| Example 147 | Compound 1-30 | Compound 2-36 | 3.53 | 21.58 | 273 | Red |
| Example 148 | Compound 1-30 | Compound 2-27 | 3.57 | 22.34 | 288 | Red |
| Example 149 | Compound 1-30 | Compound 2-54 | 3.58 | 23.30 | 260 | Red |
| Example 150 | Compound 1-30 | Compound 2-69 | 3.59 | 22.30 | 274 | Red |
| Example 151 | Compound 1-31 | Compound 2-11 | 3.58 | 22.96 | 277 | Red |
| Example 152 | Compound 1-31 | Compound 2-37 | 3.58 | 21.09 | 293 | Red |
| Example 153 | Compound 1-31 | Compound 2-28 | 3.59 | 20.76 | 270 | Red |
| Example 154 | Compound 1-31 | Compound 2-55 | 3.61 | 20.46 | 282 | Red |
| Example 155 | Compound 1-31 | Compound 2-70 | 3.59 | 20.36 | 276 | Red |
| Example 156 | Compound 1-32 | Compound 2-12 | 3.67 | 20.76 | 275 | Red |
| Example 157 | Compound 1-32 | Compound 2-20 | 3.58 | 20.57 | 273 | Red |
| Example 158 | Compound 1-32 | Compound 2-43 | 3.66 | 20.32 | 288 | Red |
| Example 159 | Compound 1-32 | Compound 2-56 | 3.59 | 20.68 | 260 | Red |
| Example 160 | Compound 1-32 | Compound 2-60 | 3.59 | 20.93 | 274 | Red |
| Example 161 | Compound 1-33 | Compound 2-13 | 3.43 | 22.96 | 293 | Red |
| Example 162 | Compound 1-33 | Compound 2-21 | 3.44 | 21.17 | 310 | Red |
| Example 163 | Compound 1-33 | Compound 2-44 | 3.33 | 22.00 | 303 | Red |
| Example 164 | Compound 1-33 | Compound 2-57 | 3.33 | 21.26 | 296 | Red |
| Example 165 | Compound 1-33 | Compound 2-62 | 3.34 | 22.61 | 306 | Red |
| Example 166 | Compound 1-34 | Compound 2-19 | 3.42 | 21.99 | 307 | Red |
| Example 167 | Compound 1-34 | Compound 2-38 | 3.47 | 21.58 | 312 | Red |
| Example 168 | Compound 1-34 | Compound 2-29 | 3.34 | 22.34 | 317 | Red |
| Example 169 | Compound 1-34 | Compound 2-58 | 3.45 | 23.30 | 297 | Red |
| Example 170 | Compound 1-34 | Compound 2-64 | 3.39 | 22.30 | 306 | Red |
| Example 171 | Compound 1-35 | Compound 2-14 | 3.66 | 22.25 | 262 | Red |
| Example 172 | Compound 1-35 | Compound 2-22 | 3.60 | 21.02 | 266 | Red |
| Example 173 | Compound 1-35 | Compound 2-40 | 3.61 | 20.73 | 288 | Red |
| Example 174 | Compound 1-35 | Compound 2-59 | 3.60 | 20.54 | 279 | Red |
| Example 175 | Compound 1-35 | Compound 2-65 | 3.61 | 20.99 | 287 | Red |
| Example 176 | Compound 1-36 | Compound 2-15 | 3.63 | 20.76 | 271 | Red |
| Example 177 | Compound 1-36 | Compound 2-39 | 3.65 | 20.57 | 284 | Red |
| Example 178 | Compound 1-36 | Compound 2-30 | 3.63 | 20.58 | 277 | Red |
| Example 179 | Compound 1-36 | Compound 2-47 | 3.59 | 20.38 | 281 | Red |
| Example 180 | Compound 1-36 | Compound 2-67 | 3.62 | 21.27 | 288 | Red |
| Example 181 | Compound 1-37 | Compound 2-1 | 3.52 | 22.15 | 276 | Red |
| Example 182 | Compound 1-37 | Compound 2-31 | 3.57 | 22.81 | 288 | Red |
| Example 183 | Compound 1-37 | Compound 2-40 | 3.53 | 22.73 | 262 | Red |
| Example 184 | Compound 1-37 | Compound 2-45 | 3.49 | 22.27 | 275 | Red |
| Example 185 | Compound 1-37 | Compound 2-60 | 3.51 | 21.51 | 292 | Red |
| Example 186 | Compound 1-38 | Compound 2-2 | 3.48 | 22.29 | 263 | Red |
| Example 187 | Compound 1-38 | Compound 2-32 | 3.50 | 21.49 | 270 | Red |
| Example 188 | Compound 1-38 | Compound 2-23 | 3.58 | 22.37 | 281 | Red |
| Example 189 | Compound 1-38 | Compound 2-46 | 3.54 | 21.86 | 264 | Red |
| Example 190 | Compound 1-38 | Compound 2-61 | 3.49 | 23.00 | 284 | Red |
| Example 191 | Compound 1-39 | Compound 2-3 | 3.64 | 21.40 | 282 | Red |
| Example 192 | Compound 1-39 | Compound 2-33 | 3.67 | 21.20 | 287 | Red |
| Example 193 | Compound 1-39 | Compound 2-24 | 3.59 | 20.84 | 292 | Red |
| Example 194 | Compound 1-39 | Compound 2-47 | 3.60 | 20.51 | 280 | Red |
| Example 195 | Compound 1-39 | Compound 2-62 | 3.61 | 20.62 | 285 | Red |
| Example 196 | Compound 1-40 | Compound 2-4 | 3.39 | 22.63 | 296 | Red |
| Example 197 | Compound 1-40 | Compound 2-16 | 3.49 | 22.88 | 311 | Red |
| Example 198 | Compound 1-40 | Compound 2-43 | 3.39 | 22.36 | 293 | Red |
| Example 199 | Compound 1-40 | Compound 2-48 | 3.42 | 22.52 | 294 | Red |
| Example 200 | Compound 1-40 | Compound 2-63 | 3.45 | 23.08 | 291 | Red |
| Example 201 | Compound 1-41 | Compound 2-1 | 3.39 | 24.32 | 296 | Red |
| Example 202 | Compound 1-41 | Compound 2-31 | 3.49 | 23.34 | 311 | Red |
| Example 203 | Compound 1-41 | Compound 2-40 | 3.39 | 23.24 | 293 | Red |
| Example 204 | Compound 1-41 | Compound 2-45 | 3.42 | 25.10 | 294 | Red |
| Example 205 | Compound 1-41 | Compound 2-60 | 3.45 | 25.08 | 291 | Red |
| Example 206 | Compound 1-42 | Compound 2-2 | 3.54 | 21.53 | 293 | Red |
| Example 207 | Compound 1-42 | Compound 2-32 | 3.55 | 22.27 | 274 | Red |
| Example 208 | Compound 1-42 | Compound 2-23 | 3.57 | 22.60 | 266 | Red |
| Example 209 | Compound 1-42 | Compound 2-46 | 3.51 | 22.84 | 273 | Red |
| Example 210 | Compound 1-42 | Compound 2-61 | 3.56 | 22.29 | 285 | Red |
| Example 211 | Compound 1-43 | Compound 2-3 | 3.62 | 21.53 | 293 | Red |
| Example 212 | Compound 1-43 | Compound 2-33 | 3.60 | 20.61 | 274 | Red |
| Example 213 | Compound 1-43 | Compound 2-24 | 3.66 | 20.34 | 266 | Red |
| Example 214 | Compound 1-43 | Compound 2-47 | 3.62 | 20.73 | 273 | Red |
| Example 215 | Compound 1-43 | Compound 2-62 | 3.59 | 20.52 | 285 | Red |
| Example 216 | Compound 1-44 | Compound 2-4 | 3.48 | 21.44 | 307 | Red |
| Example 217 | Compound 1-44 | Compound 2-16 | 3.35 | 21.72 | 314 | Red |
| Example 218 | Compound 1-44 | Compound 2-43 | 3.46 | 22.88 | 314 | Red |
| Example 219 | Compound 1-44 | Compound 2-48 | 3.47 | 21.61 | 297 | Red |
| Example 220 | Compound 1-44 | Compound 2-63 | 3.49 | 21.39 | 301 | Red |
| Example 221 | Compound 1-45 | Compound 2-5 | 3.37 | 24.49 | 295 | Red |
| Example 222 | Compound 1-45 | Compound 2-17 | 3.41 | 24.66 | 319 | Red |
| Example 223 | Compound 1-45 | Compound 2-44 | 3.33 | 24.04 | 316 | Red |
| Example 224 | Compound 1-45 | Compound 2-49 | 3.34 | 24.76 | 302 | Red |
| Example 225 | Compound 1-45 | Compound 2-64 | 3.37 | 23.62 | 294 | Red |
| Example 226 | Compound 1-46 | Compound 2-6 | 3.67 | 22.00 | 290 | Red |
| Example 227 | Compound 1-46 | Compound 2-18 | 3.62 | 20.96 | 277 | Red |
| Example 228 | Compound 1-46 | Compound 2-41 | 3.64 | 21.13 | 281 | Red |
| Example 229 | Compound 1-46 | Compound 2-50 | 3.64 | 20.71 | 287 | Red |
| Example 230 | Compound 1-46 | Compound 2-65 | 3.64 | 20.48 | 276 | Red |
| Example 231 | Compound 1-47 | Compound 2-7 | 3.49 | 21.60 | 273 | Red |
| Example 232 | Compound 1-47 | Compound 2-19 | 3.60 | 21.58 | 261 | Red |
| Example 233 | Compound 1-47 | Compound 2-42 | 3.58 | 21.52 | 283 | Red |
| Example 234 | Compound 1-47 | Compound 2-51 | 3.53 | 22.79 | 264 | Red |
| Example 235 | Compound 1-47 | Compound 2-66 | 3.61 | 22.28 | 277 | Red |
| Example 236 | Compound 1-48 | Compound 2-8 | 3.65 | 20.74 | 267 | Red |
| Example 237 | Compound 1-48 | Compound 2-34 | 3.60 | 21.00 | 270 | Red |
| Example 238 | Compound 1-48 | Compound 2-25 | 3.61 | 20.31 | 275 | Red |
| Example 239 | Compound 1-48 | Compound 2-52 | 3.66 | 21.05 | 267 | Red |
| Example 240 | Compound 1-48 | Compound 2-67 | 3.65 | 21.08 | 280 | Red |
| Example 241 | Compound 1-49 | Compound 2-9 | 3.32 | 22.28 | 295 | Red |
| Example 242 | Compound 1-49 | Compound 2-35 | 3.40 | 21.64 | 310 | Red |
| Example 243 | Compound 1-49 | Compound 2-26 | 3.33 | 22.13 | 304 | Red |
| Example 244 | Compound 1-49 | Compound 2-53 | 3.49 | 22.06 | 320 | Red |
| Example 245 | Compound 1-49 | Compound 2-68 | 3.32 | 21.82 | 299 | Red |
| Example 246 | Compound 1-50 | Compound 2-10 | 3.54 | 21.85 | 260 | Red |
| Example 247 | Compound 1-50 | Compound 2-36 | 3.49 | 23.11 | 293 | Red |
| Example 248 | Compound 1-50 | Compound 2-27 | 3.57 | 22.72 | 266 | Red |
| Example 249 | Compound 1-50 | Compound 2-54 | 3.58 | 21.36 | 272 | Red |
| Example 250 | Compound 1-50 | Compound 2-69 | 3.49 | 22.73 | 290 | Red |
| Example 251 | Compound 1-51 | Compound 2-11 | 3.67 | 22.85 | 278 | Red |
| Example 252 | Compound 1-51 | Compound 2-37 | 3.67 | 20.73 | 275 | Red |
| Example 253 | Compound 1-51 | Compound 2-28 | 3.65 | 21.19 | 275 | Red |
| Example 254 | Compound 1-51 | Compound 2-55 | 3.66 | 20.77 | 261 | Red |
| Example 255 | Compound 1-51 | Compound 2-70 | 3.61 | 20.92 | 285 | Red |
| Example 256 | Compound 1-52 | Compound 2-12 | 3.67 | 20.52 | 201 | Red |
| Example 257 | Compound 1-52 | Compound 2-20 | 3.67 | 20.73 | 203 | Red |
| Example 258 | Compound 1-52 | Compound 2-43 | 3.65 | 21.19 | 223 | Red |
| Example 259 | Compound 1-52 | Compound 2-56 | 3.66 | 20.77 | 204 | Red |
| Example 260 | Compound 1-52 | Compound 2-60 | 3.61 | 20.92 | 222 | Red |
| Example 261 | Compound 1-53 | Compound 2-13 | 3.47 | 23.22 | 270 | Red |
| Example 262 | Compound 1-53 | Compound 2-21 | 3.48 | 21.19 | 260 | Red |
| Example 263 | Compound 1-53 | Compound 2-44 | 3.50 | 23.14 | 265 | Red |
| Example 264 | Compound 1-53 | Compound 2-57 | 3.48 | 22.08 | 267 | Red |
| Example 265 | Compound 1-53 | Compound 2-62 | 3.54 | 22.88 | 276 | Red |
| Example 266 | Compound 1-54 | Compound 2-19 | 3.60 | 22.69 | 281 | Red |
| Example 267 | Compound 1-54 | Compound 2-38 | 3.57 | 21.91 | 273 | Red |
| Example 268 | Compound 1-54 | Compound 2-29 | 3.61 | 22.10 | 285 | Red |
| Example 269 | Compound 1-54 | Compound 2-58 | 3.54 | 22.32 | 288 | Red |
| Example 270 | Compound 1-54 | Compound 2-64 | 3.48 | 22.19 | 272 | Red |
| Example 271 | Compound 1-55 | Compound 2-14 | 3.65 | 22.18 | 263 | Red |
| Example 272 | Compound 1-55 | Compound 2-22 | 3.66 | 20.94 | 283 | Red |
| Example 273 | Compound 1-55 | Compound 2-40 | 3.66 | 20.41 | 280 | Red |
| Example 274 | Compound 1-55 | Compound 2-59 | 3.61 | 20.85 | 264 | Red |
| Example 275 | Compound 1-55 | Compound 2-65 | 3.62 | 20.65 | 266 | Red |
| Example 276 | Compound 1-56 | Compound 2-15 | 3.38 | 24.34 | 319 | Red |
| Example 277 | Compound 1-56 | Compound 2-39 | 3.32 | 23.78 | 313 | Red |
| Example 278 | Compound 1-56 | Compound 2-30 | 3.32 | 24.15 | 304 | Red |
| Example 279 | Compound 1-56 | Compound 2-47 | 3.34 | 23.74 | 296 | Red |
| Example 280 | Compound 1-56 | Compound 2-67 | 3.32 | 24.03 | 302 | Red |
| Example 281 | Compound 1-57 | Compound 2-1 | 3.69 | 20.02 | 201 | Red |
| Example 282 | Compound 1-57 | Compound 2-31 | 3.70 | 20.26 | 213 | Red |
| Example 283 | Compound 1-57 | Compound 2-40 | 3.65 | 20.03 | 204 | Red |
| Example 284 | Compound 1-57 | Compound 2-45 | 3.68 | 20.14 | 214 | Red |
| Example 285 | Compound 1-57 | Compound 2-60 | 3.68 | 20.37 | 215 | Red |
| Example 286 | Compound 1-58 | Compound 2-2 | 3.69 | 20.14 | 220 | Red |
| Example 287 | Compound 1-58 | Compound 2-32 | 3.67 | 20.39 | 201 | Red |
| Example 288 | Compound 1-58 | Compound 2-23 | 3.65 | 20.21 | 203 | Red |
| Example 289 | Compound 1-58 | Compound 2-46 | 3.73 | 20.03 | 202 | Red |
| Example 290 | Compound 1-58 | Compound 2-61 | 3.72 | 20.28 | 220 | Red |
| Example 291 | Compound 1-59 | Compound 2-3 | 3.63 | 20.40 | 222 | Red |
| Example 292 | Compound 1-59 | Compound 2-33 | 3.67 | 20.40 | 208 | Red |
| Example 293 | Compound 1-59 | Compound 2-24 | 3.61 | 20.37 | 212 | Red |
| Example 294 | Compound 1-59 | Compound 2-47 | 3.66 | 20.87 | 217 | Red |
| Example 295 | Compound 1-59 | Compound 2-62 | 3.62 | 21.20 | 211 | Red |
| Example 296 | Compound 1-60 | Compound 2-4 | 3.67 | 20.48 | 219 | Red |
| Example 297 | Compound 1-60 | Compound 2-16 | 3.60 | 20.94 | 215 | Red |
| Example 298 | Compound 1-60 | Compound 2-43 | 3.62 | 20.73 | 204 | Red |
| Example 299 | Compound 1-60 | Compound 2-48 | 3.59 | 20.95 | 205 | Red |
| Example 300 | Compound 1-60 | Compound 2-63 | 3.66 | 20.51 | 215 | Red |
| Example 301 | Compound 1-61 | Compound 2-1 | 3.63 | 21.86 | 287 | Red |
| Example 302 | Compound 1-61 | Compound 2-31 | 3.65 | 20.77 | 292 | Red |
| Example 303 | Compound 1-61 | Compound 2-40 | 3.60 | 21.28 | 260 | Red |
| Example 304 | Compound 1-61 | Compound 2-45 | 3.60 | 20.63 | 281 | Red |
| Example 305 | Compound 1-61 | Compound 2-60 | 3.63 | 21.05 | 293 | Red |
| Example 306 | Compound 1-62 | Compound 2-2 | 3.61 | 20.80 | 292 | Red |
| Example 307 | Compound 1-62 | Compound 2-32 | 3.62 | 20.89 | 263 | Red |
| Example 308 | Compound 1-62 | Compound 2-23 | 3.62 | 20.36 | 284 | Red |
| Example 309 | Compound 1-62 | Compound 2-46 | 3.59 | 20.45 | 280 | Red |
| Example 310 | Compound 1-62 | Compound 2-61 | 3.62 | 21.00 | 275 | Red |
| Example 311 | Compound 1-63 | Compound 2-3 | 3.34 | 24.05 | 296 | Red |
| Example 312 | Compound 1-63 | Compound 2-33 | 3.46 | 23.61 | 304 | Red |
| Example 313 | Compound 1-63 | Compound 2-24 | 3.40 | 24.38 | 312 | Red |
| Example 314 | Compound 1-63 | Compound 2-47 | 3.48 | 23.17 | 302 | Red |
| Example 315 | Compound 1-63 | Compound 2-62 | 3.47 | 24.86 | 293 | Red |
| Example 316 | Compound 1-64 | Compound 2-4 | 3.48 | 21.85 | 319 | Red |
| Example 317 | Compound 1-64 | Compound 2-16 | 3.48 | 21.72 | 299 | Red |
| Example 318 | Compound 1-64 | Compound 2-43 | 3.35 | 23.23 | 318 | Red |
| Example 319 | Compound 1-64 | Compound 2-48 | 3.41 | 22.32 | 300 | Red |
| Example 320 | Compound 1-64 | Compound 2-63 | 3.38 | 21.49 | 305 | Red |
| Example 321 | Compound 1-65 | Compound 2-5 | 3.52 | 22.36 | 271 | Red |
| Example 322 | Compound 1-65 | Compound 2-17 | 3.49 | 21.44 | 279 | Red |
| Example 323 | Compound 1-65 | Compound 2-44 | 3.53 | 22.72 | 277 | Red |
| Example 324 | Compound 1-65 | Compound 2-49 | 3.53 | 22.84 | 272 | Red |
| Example 325 | Compound 1-65 | Compound 2-64 | 3.57 | 21.96 | 278 | Red |
| Example 326 | Compound 1-66 | Compound 2-6 | 3.47 | 21.28 | 289 | Red |
| Example 327 | Compound 1-66 | Compound 2-18 | 3.51 | 22.49 | 279 | Red |
| Example 328 | Compound 1-66 | Compound 2-41 | 3.61 | 23.30 | 277 | Red |
| Example 329 | Compound 1-66 | Compound 2-50 | 3.47 | 22.97 | 283 | Red |
| Example 330 | Compound 1-66 | Compound 2-65 | 3.54 | 21.14 | 288 | Red |
| Example 331 | Compound 1-67 | Compound 2-7 | 3.67 | 20.15 | 219 | Red |
| Example 332 | Compound 1-67 | Compound 2-19 | 3.67 | 20.07 | 224 | Red |
| Example 333 | Compound 1-67 | Compound 2-42 | 3.73 | 20.11 | 212 | Red |
| Example 334 | Compound 1-67 | Compound 2-51 | 3.71 | 20.34 | 221 | Red |
| Example 335 | Compound 1-67 | Compound 2-66 | 3.66 | 20.01 | 215 | Red |
| Example 336 | Compound 1-68 | Compound 2-8 | 3.68 | 20.21 | 219 | Red |
| Example 337 | Compound 1-68 | Compound 2-34 | 3.67 | 20.12 | 213 | Red |
| Example 338 | Compound 1-68 | Compound 2-25 | 3.72 | 20.22 | 206 | Red |
| Example 339 | Compound 1-68 | Compound 2-52 | 3.69 | 20.11 | 205 | Red |
| Example 340 | Compound 1-68 | Compound 2-67 | 3.71 | 20.06 | 224 | Red |
| Example 341 | Compound 1-69 | Compound 2-9 | 3.59 | 21.12 | 274 | Red |
| Example 342 | Compound 1-69 | Compound 2-35 | 3.66 | 20.53 | 263 | Red |
| Example 343 | Compound 1-69 | Compound 2-26 | 3.58 | 21.25 | 277 | Red |
| Example 344 | Compound 1-69 | Compound 2-53 | 3.64 | 20.89 | 282 | Red |
| Example 345 | Compound 1-69 | Compound 2-68 | 3.62 | 20.80 | 279 | Red |
| Example 346 | Compound 1-70 | Compound 2-10 | 3.65 | 21.29 | 292 | Red |
| Example 347 | Compound 1-70 | Compound 2-36 | 3.66 | 20.60 | 273 | Red |
| Example 348 | Compound 1-70 | Compound 2-27 | 3.59 | 20.71 | 290 | Red |
| Example 349 | Compound 1-70 | Compound 2-54 | 3.62 | 20.90 | 277 | Red |
| Example 350 | Compound 1-70 | Compound 2-69 | 3.59 | 20.87 | 263 | Red |
| Example 351 | Compound 1-71 | Compound 2-11 | 3.37 | 24.46 | 319 | Red |
| Example 352 | Compound 1-71 | Compound 2-37 | 3.32 | 23.25 | 307 | Red |
| Example 353 | Compound 1-71 | Compound 2-28 | 3.42 | 23.50 | 300 | Red |
| Example 354 | Compound 1-71 | Compound 2-55 | 3.41 | 23.96 | 302 | Red |
| Example 355 | Compound 1-71 | Compound 2-70 | 3.42 | 23.58 | 320 | Red |
| Example 356 | Compound 1-72 | Compound 2-12 | 3.32 | 23.99 | 309 | Red |
| Example 357 | Compound 1-72 | Compound 2-20 | 3.47 | 24.22 | 305 | Red |
| Example 358 | Compound 1-72 | Compound 2-43 | 3.36 | 24.18 | 309 | Red |
| Example 359 | Compound 1-72 | Compound 2-56 | 3.33 | 24.85 | 293 | Red |
| Example 360 | Compound 1-72 | Compound 2-60 | 3.35 | 23.40 | 313 | Red |
| Example 361 | Compound 1-73 | Compound 2-13 | 3.69 | 20.45 | 204 | Red |
| Example 362 | Compound 1-73 | Compound 2-21 | 3.66 | 20.46 | 220 | Red |
| Example 363 | Compound 1-73 | Compound 2-44 | 3.66 | 20.48 | 215 | Red |
| Example 364 | Compound 1-73 | Compound 2-57 | 3.73 | 20.33 | 203 | Red |
| Example 365 | Compound 1-73 | Compound 2-62 | 3.69 | 20.37 | 211 | Red |
| Example 366 | Compound 1-74 | Compound 2-19 | 3.69 | 20.42 | 202 | Red |
| Example 367 | Compound 1-74 | Compound 2-38 | 3.70 | 20.43 | 225 | Red |
| Example 368 | Compound 1-74 | Compound 2-29 | 3.70 | 20.02 | 220 | Red |
| Example 369 | Compound 1-74 | Compound 2-58 | 3.68 | 20.30 | 213 | Red |
| Example 370 | Compound 1-74 | Compound 2-64 | 3.69 | 20.22 | 206 | Red |
| Example 371 | Compound 1-75 | Compound 2-14 | 3.67 | 20.70 | 209 | Red |
| Example 372 | Compound 1-75 | Compound 2-22 | 3.64 | 20.91 | 215 | Red |
| Example 373 | Compound 1-75 | Compound 2-40 | 3.64 | 20.95 | 219 | Red |
| Example 374 | Compound 1-75 | Compound 2-59 | 3.59 | 20.65 | 218 | Red |
| Example 375 | Compound 1-75 | Compound 2-65 | 3.59 | 20.67 | 214 | Red |
| Example 376 | Compound 1-76 | Compound 2-15 | 3.66 | 20.70 | 215 | Red |
| Example 377 | Compound 1-76 | Compound 2-39 | 3.63 | 21.11 | 223 | Red |
| Example 378 | Compound 1-76 | Compound 2-30 | 3.61 | 21.10 | 218 | Red |
| Example 379 | Compound 1-76 | Compound 2-47 | 3.62 | 21.25 | 218 | Red |
| Example 380 | Compound 1-76 | Compound 2-67 | 3.66 | 20.84 | 218 | Red |
| Example 381 | Compound 1-77 | Compound 2-1 | 3.66 | 20.70 | 263 | Red |
| Example 382 | Compound 1-77 | Compound 2-31 | 3.63 | 21.11 | 276 | Red |
| Example 383 | Compound 1-77 | Compound 2-40 | 3.61 | 21.10 | 284 | Red |
| Example 384 | Compound 1-77 | Compound 2-45 | 3.62 | 21.25 | 268 | Red |
| Example 385 | Compound 1-77 | Compound 2-60 | 3.66 | 20.84 | 279 | Red |
| Example 386 | Compound 1-78 | Compound 2-2 | 3.48 | 22.83 | 317 | Red |
| Example 387 | Compound 1-78 | Compound 2-32 | 3.41 | 21.79 | 299 | Red |
| Example 388 | Compound 1-78 | Compound 2-23 | 3.37 | 21.24 | 320 | Red |
| Example 389 | Compound 1-78 | Compound 2-46 | 3.34 | 21.46 | 312 | Red |
| Example 390 | Compound 1-78 | Compound 2-61 | 3.45 | 22.13 | 307 | Red |
| Example 391 | Compound 1-79 | Compound 2-3 | 3.57 | 22.83 | 291 | Red |
| Example 392 | Compound 1-79 | Compound 2-33 | 3.50 | 21.79 | 268 | Red |
| Example 393 | Compound 1-79 | Compound 2-24 | 3.61 | 21.24 | 273 | Red |
| Example 394 | Compound 1-79 | Compound 2-47 | 3.57 | 21.46 | 260 | Red |
| Example 395 | Compound 1-79 | Compound 2-62 | 3.56 | 22.13 | 270 | Red |

TABLE 2

| | | | Voltage | Efficiency | Lifespan | Emission |
|---|---|---|---|---|---|---|
| Category | First host | Second host | (V) | (cd/A) | T95(hr) | color |
| Comparative | Compound B-1 | Compound 2-1 | 4.14 | 15.81 | 105 | Red |
| Example 1 | | | | | | |
| Comparative | Compound B-1 | Compound 2-31 | 4.13 | 15.18 | 91 | Red |
| Example 2 | | | | | | |
| Comparative | Compound B-1 | Compound 2-40 | 4.06 | 15.97 | 122 | Red |
| Example 3 | | | | | | |
| Comparative | Compound B-1 | Compound 2-45 | 4.07 | 15.80 | 113 | Red |
| Example 4 | | | | | | |
| Comparative | Compound B-1 | Compound 2-60 | 4.13 | 15.17 | 125 | Red |
| Example 5 | | | | | | |
| Comparative | Compound B-2 | Compound 2-2 | 4.13 | 15.78 | 96 | Red |
| Example 6 | | | | | | |
| Comparative | Compound B-2 | Compound 2-32 | 4.13 | 16.04 | 92 | Red |
| Example 7 | | | | | | |
| Comparative | Compound B-2 | Compound 2-23 | 4.13 | 14.97 | 105 | Red |
| Example 8 | | | | | | |
| Comparative | Compound B-2 | Compound 2-46 | 4.05 | 14.52 | 111 | Red |
| Example 9 | | | | | | |
| Comparative | Compound B-2 | Compound 2-61 | 4.09 | 14.56 | 93 | Red |
| Example 10 | | | | | | |
| Comparative | Compound B-3 | Compound 2-3 | 4.17 | 15.81 | 85 | Red |
| Example 11 | | | | | | |
| Comparative | Compound B-3 | Compound 2-33 | 4.20 | 15.18 | 74 | Red |
| Example 12 | | | | | | |
| Comparative | Compound B-3 | Compound 2-24 | 4.20 | 15.97 | 85 | Red |
| Example 13 | | | | | | |
| Comparative | Compound B-3 | Compound 2-47 | 4.20 | 15.80 | 87 | Red |
| Example 14 | | | | | | |
| Comparative | Compound B-3 | Compound 2-62 | 4.23 | 15.17 | 80 | Red |
| Example 15 | | | | | | |
| Comparative | Compound B-4 | Compound 2-4 | 4.17 | 15.78 | 95 | Red |
| Example 16 | | | | | | |
| Comparative | Compound B-4 | Compound 2-16 | 4.21 | 16.04 | 76 | Red |
| Example 17 | | | | | | |
| Comparative | Compound B-4 | Compound 2-43 | 4.11 | 14.97 | 73 | Red |
| Example 18 | | | | | | |
| Comparative | Compound B-4 | Compound 2-48 | 4.10 | 14.52 | 87 | Red |
| Example 19 | | | | | | |
| Comparative | Compound B-4 | Compound 2-63 | 4.14 | 14.56 | 86 | Red |
| Example 20 | | | | | | |
| Comparative | Compound B-5 | Compound 2-5 | 3.99 | 17.81 | 137 | Red |
| Example 21 | | | | | | |
| Comparative | Compound B-5 | Compound 2-17 | 3.94 | 17.20 | 137 | Red |
| Example 22 | | | | | | |
| Comparative | Compound B-5 | Compound 2-44 | 3.92 | 17.17 | 103 | Red |
| Example 23 | | | | | | |
| Comparative | Compound B-5 | Compound 2-49 | 3.95 | 16.99 | 107 | Red |
| Example 24 | | | | | | |
| Comparative | Compound B-5 | Compound 2-64 | 3.93 | 17.86 | 127 | Red |
| Example 25 | | | | | | |
| Comparative | Compound B-6 | Compound 2-6 | 3.90 | 17.30 | 114 | Red |
| Example 26 | | | | | | |
| Comparative | Compound B-6 | Compound 2-18 | 3.88 | 17.82 | 110 | Red |
| Example 27 | | | | | | |
| Comparative | Compound B-6 | Compound 2-41 | 3.94 | 17.40 | 108 | Red |
| Example 28 | | | | | | |
| Comparative | Compound B-6 | Compound 2-50 | 3.94 | 17.50 | 106 | Red |
| Example 29 | | | | | | |
| Comparative | Compound B-6 | Compound 2-65 | 3.89 | 18.00 | 121 | Red |
| Example 30 | | | | | | |
| Comparative | Compound B-7 | Compound 2-7 | 4.15 | 16.48 | 127 | Red |
| Example 31 | | | | | | |
| Comparative | Compound B-7 | Compound 2-19 | 4.15 | 16.46 | 158 | Red |
| Example 32 | | | | | | |
| Comparative | Compound B-7 | Compound 2-42 | 4.08 | 16.62 | 141 | Red |
| Example 33 | | | | | | |
| Comparative | Compound B-7 | Compound 2-51 | 4.17 | 17.13 | 115 | Red |
| Example 34 | | | | | | |
| Comparative | Compound B-7 | Compound 2-66 | 4.07 | 16.57 | 121 | Red |
| Example 35 | | | | | | |
| Comparative | Compound B-8 | Compound 2-8 | 4.11 | 16.87 | 154 | Red |
| Example 36 | | | | | | |
| Comparative | Compound B-8 | Compound 2-34 | 4.16 | 17.18 | 120 | Red |
| Example 37 | | | | | | |
| Comparative | Compound B-8 | Compound 2-25 | 4.07 | 16.96 | 121 | Red |
| Example 38 | | | | | | |
| Comparative | Compound B-8 | Compound 2-52 | 4.17 | 16.86 | 114 | Red |
| Example 39 | | | | | | |
| Comparative | Compound B-8 | Compound 2-67 | 4.05 | 16.51 | 118 | Red |
| Example 40 | | | | | | |
| Comparative | Compound B-9 | Compound 2-9 | 4.15 | 14.94 | 117 | Red |
| Example 41 | | | | | | |
| Comparative | Compound B-9 | Compound 2-35 | 4.15 | 16.12 | 98 | Red |
| Example 42 | | | | | | |
| Comparative | Compound B-9 | Compound 2-26 | 4.08 | 16.55 | 125 | Red |
| Example 43 | | | | | | |
| Comparative | Compound B-9 | Compound 2-53 | 4.17 | 15.44 | 108 | Red |
| Example 44 | | | | | | |
| Comparative | Compound B-9 | Compound 2-68 | 4.07 | 14.74 | 124 | Red |
| Example 45 | | | | | | |
| Comparative | Compound B-10 | Compound 2-10 | 4.11 | 16.17 | 113 | Red |
| Example 46 | | | | | | |
| Comparative | Compound B-10 | Compound 2-36 | 4.16 | 15.33 | 93 | Red |
| Example 47 | | | | | | |
| Comparative | Compound B-10 | Compound 2-27 | 4.07 | 16.55 | 125 | Red |
| Example 48 | | | | | | |
| Comparative | Compound B-10 | Compound 2-54 | 4.17 | 15.07 | 92 | Red |
| Example 49 | | | | | | |
| Comparative | Compound B-10 | Compound 2-69 | 4.05 | 14.93 | 105 | Red |
| Example 50 | | | | | | |
| Comparative | Compound B-11 | Compound 2-11 | 3.95 | 17.75 | 150 | Red |
| Example 51 | | | | | | |
| Comparative | Compound B-11 | Compound 2-37 | 3.92 | 17.43 | 147 | Red |
| Example 52 | | | | | | |
| Comparative | Compound B-11 | Compound 2-28 | 3.94 | 16.97 | 168 | Red |
| Example 53 | | | | | | |
| Comparative | Compound B-11 | Compound 2-55 | 3.88 | 17.64 | 155 | Red |
| Example 54 | | | | | | |
| Comparative | Compound B-11 | Compound 2-70 | 3.94 | 17.58 | 166 | Red |
| Example 55 | | | | | | |
| Comparative | Compound B-12 | Compound 2-12 | 3.92 | 17.15 | 157 | Red |
| Example 56 | | | | | | |
| Comparative | Compound B-12 | Compound 2-20 | 3.90 | 17.35 | 150 | Red |
| Example 57 | | | | | | |
| Comparative | Compound B-12 | Compound 2-43 | 3.90 | 17.27 | 165 | Red |
| Example 58 | | | | | | |
| Comparative | Compound B-12 | Compound 2-56 | 3.92 | 17.01 | 156 | Red |
| Example 59 | | | | | | |
| Comparative | Compound B-12 | Compound 2-60 | 3.92 | 17.59 | 150 | Red |
| Example 60 | | | | | | |

TABLE 3

| | | | Voltage | Efficiency | Lifespan | Emission |
|---|---|---|---|---|---|---|
| Category | First host | Second host | (V) | (cd/A) | T95(hr) | color |
| Comparative | Compound 1-1 | Compound C-1 | 4.16 | 16.87 | 104 | Red |
| Example 61 | | | | | | |
| Comparative | Compound 1-29 | Compound C-1 | 4.07 | 17.19 | 116 | Red |
| Example 62 | | | | | | |
| Comparative | Compound 1-13 | Compound C-1 | 4.11 | 16.85 | 120 | Red |
| Example 63 | | | | | | |
| Comparative | Compound 1-25 | Compound C-1 | 4.05 | 16.73 | 152 | Red |
| Example 64 | | | | | | |
| Comparative | Compound 1-41 | Compound C-1 | 4.16 | 15.35 | 114 | Red |
| Example 65 | | | | | | |
| Comparative | Compound 1-47 | Compound C-1 | 4.07 | 15.76 | 114 | Red |
| Example 66 | | | | | | |
| Comparative | Compound 1-52 | Compound C-1 | 4.11 | 16.47 | 97 | Red |
| Example 67 | | | | | | |
| Comparative | Compound 1-64 | Compound C-1 | 4.05 | 16.22 | 125 | Red |
| Example 68 | | | | | | |
| Comparative | Compound 1-2 | Compound C-2 | 3.91 | 17.95 | 154 | Red |
| Example 69 | | | | | | |
| Comparative | Compound 1-14 | Compound C-2 | 3.88 | 17.44 | 103 | Red |
| Example 70 | | | | | | |
| Comparative | Compound 1-26 | Compound C-2 | 3.90 | 17.62 | 106 | Red |
| Example 71 | | | | | | |
| Comparative | Compound 1-30 | Compound C-2 | 3.90 | 17.98 | 136 | Red |
| Example 72 | | | | | | |
| Comparative | Compound 1-42 | Compound C-2 | 3.91 | 17.95 | 168 | Red |
| Example 73 | | | | | | |
| Comparative | Compound 1-48 | Compound C-2 | 3.88 | 17.44 | 163 | Red |
| Example 74 | | | | | | |
| Comparative | Compound 1-53 | Compound C-2 | 3.90 | 17.62 | 162 | Red |
| Example 75 | | | | | | |
| Comparative | Compound 1-65 | Compound C-2 | 3.90 | 17.98 | 148 | Red |
| Example 76 | | | | | | |
| Comparative | Compound 1-3 | Compound C-3 | 4.12 | 14.57 | 76 | Red |
| Example 77 | | | | | | |
| Comparative | Compound 1-15 | Compound C-3 | 4.21 | 15.00 | 91 | Red |
| Example 78 | | | | | | |
| Comparative | Compound 1-27 | Compound C-3 | 4.15 | 16.49 | 85 | Red |
| Example 79 | | | | | | |
| Comparative | Compound 1-31 | Compound C-3 | 4.15 | 15.69 | 92 | Red |
| Example 80 | | | | | | |
| Comparative | Compound 1-43 | Compound C-3 | 4.11 | 16.56 | 154 | Red |
| Example 81 | | | | | | |
| Comparative | Compound 1-49 | Compound C-3 | 4.05 | 16.50 | 103 | Red |
| Example 82 | | | | | | |
| Comparative | Compound 1-54 | Compound C-3 | 4.11 | 16.73 | 106 | Red |
| Example 83 | | | | | | |
| Comparative | Compound 1-66 | Compound C-3 | 4.14 | 16.71 | 136 | Red |
| Example 84 | | | | | | |
| Comparative | Compound 1-4 | Compound C-4 | 3.89 | 17.31 | 167 | Red |
| Example 85 | | | | | | |
| Comparative | Compound 1-16 | Compound C-4 | 3.89 | 16.99 | 170 | Red |
| Example 86 | | | | | | |
| Comparative | Compound 1-32 | Compound C-4 | 3.89 | 17.45 | 155 | Red |
| Example 87 | | | | | | |
| Comparative | Compound 1-44 | Compound C-4 | 3.90 | 17.06 | 150 | Red |
| Example 88 | | | | | | |
| Comparative | Compound 1-50 | Compound C-4 | 3.92 | 17.31 | 158 | Red |
| Example 89 | | | | | | |
| Comparative | Compound 1-52 | Compound C-4 | 3.94 | 16.99 | 116 | Red |
| Example 90 | | | | | | |
| Comparative | Compound 1-55 | Compound C-4 | 3.93 | 17.45 | 107 | Red |
| Example 91 | | | | | | |
| Comparative | Compound 1-67 | Compound C-4 | 3.95 | 17.06 | 127 | Red |
| Example 92 | | | | | | |
| Comparative | Compound 1-5 | Compound C-5 | 3.94 | 17.65 | 162 | Red |
| Example 93 | | | | | | |
| Comparative | Compound 1-17 | Compound C-5 | 3.88 | 17.82 | 155 | Red |
| Example 94 | | | | | | |
| Comparative | Compound 1-33 | Compound C-5 | 3.90 | 17.32 | 169 | Red |
| Example 95 | | | | | | |
| Comparative | Compound 1-45 | Compound C-5 | 3.92 | 17.99 | 169 | Red |
| Example 96 | | | | | | |
| Comparative | Compound 1-51 | Compound C-5 | 3.91 | 17.88 | 164 | Red |
| Example 97 | | | | | | |
| Comparative | Compound 1-53 | Compound C-5 | 3.91 | 16.90 | 165 | Red |
| Example 98 | | | | | | |
| Comparative | Compound 1-56 | Compound C-5 | 3.89 | 17.64 | 165 | Red |
| Example 99 | | | | | | |
| Comparative | Compound 1-68 | Compound C-5 | 3.91 | 17.21 | 156 | Red |
| Example 100 | | | | | | |
| Comparative | Compound 1-6 | Compound C-6 | 4.11 | 16.64 | 150 | Red |
| Example 101 | | | | | | |
| Comparative | Compound 1-18 | Compound C-6 | 4.07 | 16.64 | 159 | Red |
| Example 102 | | | | | | |
| Comparative | Compound 1-28 | Compound C-6 | 4.15 | 16.68 | 148 | Red |
| Example 103 | | | | | | |
| Comparative | Compound 1-34 | Compound C-6 | 4.12 | 17.07 | 104 | Red |
| Example 104 | | | | | | |
| Comparative | Compound 1-46 | Compound C-6 | 4.16 | 16.70 | 140 | Red |
| Example 105 | | | | | | |
| Comparative | Compound 1-54 | Compound C-6 | 4.06 | 16.83 | 124 | Red |
| Example 106 | | | | | | |
| Comparative | Compound 1-57 | Compound C-6 | 4.06 | 16.52 | 159 | Red |
| Example 107 | | | | | | |
| Comparative | Compound 1-69 | Compound C-6 | 4.13 | 16.86 | 119 | Red |
| Example 108 | | | | | | |
| Comparative | Compound 1-7 | Compound C-7 | 3.98 | 17.19 | 113 | Red |
| Example 109 | | | | | | |
| Comparative | Compound 1-19 | Compound C-7 | 3.88 | 17.79 | 152 | Red |
| Example 110 | | | | | | |
| Comparative | Compound 1-35 | Compound C-7 | 3.88 | 17.87 | 127 | Red |
| Example 111 | | | | | | |
| Comparative | Compound 1-38 | Compound C-7 | 3.92 | 17.20 | 148 | Red |
| Example 112 | | | | | | |
| Comparative | Compound 1-41 | Compound C-7 | 3.91 | 17.12 | 132 | Red |
| Example 113 | | | | | | |
| Comparative | Compound 1-47 | Compound C-7 | 3.88 | 17.67 | 144 | Red |
| Example 114 | | | | | | |
| Comparative | Compound 1-58 | Compound C-7 | 3.95 | 17.40 | 134 | Red |
| Example 115 | | | | | | |
| Comparative | Compound 1-70 | Compound C-7 | 3.89 | 17.20 | 151 | Red |
| Example 116 | | | | | | |
| Comparative | Compound 1-8 | Compound C-8 | 4.15 | 15.00 | 71 | Red |
| Example 117 | | | | | | |
| Comparative | Compound 1-20 | Compound C-8 | 4.09 | 15.61 | 81 | Red |
| Example 118 | | | | | | |
| Comparative | Compound 1-36 | Compound C-8 | 4.19 | 14.72 | 86 | Red |
| Example 119 | | | | | | |
| Comparative | Compound 1-39 | Compound C-8 | 4.18 | 15.41 | 74 | Red |
| Example 120 | | | | | | |
| Comparative | Compound 1-42 | Compound C-8 | 3.92 | 17.14 | 170 | Red |
| Example 121 | | | | | | |
| Comparative | Compound 1-48 | Compound C-8 | 3.93 | 17.91 | 156 | Red |
| Example 122 | | | | | | |
| Comparative | Compound 1-59 | Compound C-8 | 3.95 | 17.91 | 158 | Red |
| Example 123 | | | | | | |
| Comparative | Compound 1-71 | Compound C-8 | 3.91 | 17.09 | 168 | Red |
| Example 124 | | | | | | |
| Comparative | Compound 1-9 | Compound C-9 | 3.94 | 16.99 | 143 | Red |
| Example 125 | | | | | | |
| Comparative | Compound 1-21 | Compound C-9 | 3.91 | 16.98 | 142 | Red |
| Example 126 | | | | | | |
| Comparative | Compound 1-37 | Compound C-9 | 3.94 | 17.89 | 102 | Red |
| Example 127 | | | | | | |
| Comparative | Compound 1-43 | Compound C-9 | 3.91 | 17.47 | 103 | Red |
| Example 128 | | | | | | |
| Comparative | Compound 1-49 | Compound C-9 | 3.88 | 17.31 | 151 | Red |
| Example 129 | | | | | | |
| Comparative | Compound 1-60 | Compound C-9 | 3.93 | 17.85 | 112 | Red |
| Example 130 | | | | | | |
| Comparative | Compound 1-72 | Compound C-9 | 3.94 | 17.03 | 137 | Red |
| Example 131 | | | | | | |
| Comparative | Compound 1-76 | Compound C-9 | 3.90 | 17.42 | 123 | Red |
| Example 132 | | | | | | |
| Comparative | Compound 1-10 | Compound C-10 | 3.96 | 17.44 | 163 | Red |
| Example 133 | | | | | | |
| Comparative | Compound 1-22 | Compound C-10 | 3.91 | 17.96 | 162 | Red |
| Example 134 | | | | | | |
| Comparative | Compound 1-38 | Compound C-10 | 3.94 | 17.05 | 145 | Red |
| Example 135 | | | | | | |
| Comparative | Compound 1-44 | Compound C-10 | 3.91 | 17.88 | 167 | Red |
| Example 136 | | | | | | |
| Comparative | Compound 1-50 | Compound C-10 | 3.88 | 16.97 | 155 | Red |
| Example 137 | | | | | | |
| Comparative | Compound 1-61 | Compound C-10 | 3.92 | 17.73 | 168 | Red |
| Example 138 | | | | | | |
| Comparative | Compound 1-73 | Compound C-10 | 3.90 | 17.05 | 146 | Red |
| Example 139 | | | | | | |
| Comparative | Compound 1-77 | Compound C-10 | 3.94 | 17.12 | 158 | Red |
| Example 140 | | | | | | |
| Comparative | Compound 1-11 | Compound C-11 | 4.14 | 15.64 | 116 | Red |
| Example 141 | | | | | | |
| Comparative | Compound 1-23 | Compound C-11 | 4.17 | 15.97 | 113 | Red |
| Example 142 | | | | | | |
| Comparative | Compound 1-39 | Compound C-11 | 4.16 | 15.09 | 113 | Red |
| Example 143 | | | | | | |
| Comparative | Compound 1-45 | Compound C-11 | 4.09 | 14.86 | 91 | Red |
| Example 144 | | | | | | |
| Comparative | Compound 1-51 | Compound C-11 | 4.16 | 15.48 | 116 | Red |
| Example 145 | | | | | | |
| Comparative | Compound 1-62 | Compound C-11 | 4.09 | 16.28 | 125 | Red |
| Example 146 | | | | | | |
| Comparative | Compound 1-74 | Compound C-11 | 4.13 | 15.61 | 125 | Red |
| Example 147 | | | | | | |
| Comparative | Compound 1-78 | Compound C-11 | 4.14 | 15.44 | 101 | Red |
| Example 148 | | | | | | |
| Comparative | Compound 1-12 | Compound C-12 | 4.10 | 16.48 | 138 | Red |
| Example 149 | | | | | | |
| Comparative | Compound 1-24 | Compound C-12 | 4.08 | 16.59 | 136 | Red |
| Example 150 | | | | | | |
| Comparative | Compound 1-28 | Compound C-12 | 4.16 | 16.80 | 128 | Red |
| Example 151 | | | | | | |
| Comparative | Compound 1-40 | Compound C-12 | 4.07 | 16.70 | 156 | Red |
| Example 152 | | | | | | |
| Comparative | Compound 1-46 | Compound C-12 | 4.16 | 16.90 | 116 | Red |
| Example 153 | | | | | | |
| Comparative | Compound 1-63 | Compound C-12 | 4.06 | 16.86 | 129 | Red |
| Example 154 | | | | | | |
| Comparative | Compound 1-75 | Compound C-12 | 4.09 | 16.53 | 141 | Red |
| Example 155 | | | | | | |
| Comparative | Compound 1-79 | Compound C-12 | 4.14 | 17.02 | 135 | Red |
| Example 156 | | | | | | |

When a current was applied to the organic light emitting devices manufactured according to Examples 1 to 395 and Comparative Examples 1 to 156, the results of Tables 1 to 3 above were obtained. In the red organic light emitting device of Comparative Example 1, a material that has been widely used conventionally was used, and it is a structure using Compound EB-1 as an electron blocking layer and Compound Dp-7 as a dopant material for the red light emitting layer.

As shown in the above Table 1, the organic light-emitting devices of the Examples, which were co-deposited and used both the first compound of Chemical Formula 1 and the second compound of Chemical Formula 2 as host materials for the red light-emitting layer according to the present disclosure, shows excellent characteristics of reducing the driving voltage and increasing the efficiency and lifespan compared to the organic light-emitting devices of Comparative Example 1 and 156, which adopted a combination of other host materials instead of the above combinations of the first compound and the second compound.

Specifically, the organic light emitting devices of Examples 1 to 395 according to the present disclosure maintain a lower driving voltage and had the efficiency improved by about 11% to about 73%, and the lifespan improved by about 19% to about 439%, compared to the organic light emitting devices of Comparative Examples 1 to 60 using the second compound and a compound having a structure different from that of the first compound. Further, the organic light emitting devices of Examples 1 to 395 according to the present disclosure maintain a lower driving voltage and had the efficiency improved by about 11% to about 72%, and the lifespan improved by about 18% to about 420%, compared to the organic light emitting devices of Comparative Examples 61 to 156 using the first compound and a compound having a structure different from that of the second compound.

From these results, it can be confirmed that the increase in efficiency and lifespan while maintaining the low driving voltage of the organic light emitting device is because the combination of the compound of Chemical Formula 1 as a first host and the compound of Chemical Formula 2 as a second host facilitates energy transfer to the red dopant in the red light emitting layer. It can be also confirmed that the combination of the compounds of the Examples according to the present disclosure, that is, the combination of the compound of Chemical Formula 1 and the compound of Chemical Formula 2, facilitates electron-hole bonding in a more stable balance to form excitons in the light emitting layer, thereby greatly increasing the efficiency and lifespan. In conclusion, when the compound of Chemical Formula 1 and the compound of Chemical Formula 2 of the present disclosure are combined and co-deposited to be used as a host for the red light emitting layer, it is possible to significantly improve the lifespan while maintaining the low driving voltage and high luminous efficiency of the organic light emitting device.

[DESCRIPTION OF SYMBOLS]

| | |
|---|---|
| 1: Substrate | 2: Anode |
| 3: Light emitting layer | 4: Cathode |
| 5: Hole injection layer | 6: Hole transport layer |
| 7: Electron blocking layer | 8: Hole blocking layer |
| 9: Electron injection and transport layer. | |

1. An organic light emitting device, comprising:
an anode;
a cathode; and
a light emitting layer that is between the anode and the cathode,
wherein the light emitting layer comprises a compound of the following Chemical Formula 1 and a compound of the following Chemical Formula 2: 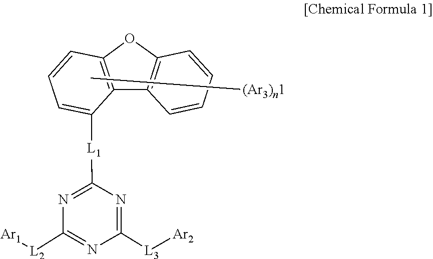
wherein in Chemical Formula 1:
L₁ to L₃ are each independently a single bond or substituted or unsubstituted C₆₋₆₀ arylene;
Ar₁ and Ar₂ are each independently a substituted or unsubstituted C₆₋₆₀ aryl or substituted or unsubstituted C₂₋₆₀ heteroaryl containing at least one heteroatom selected from the group consisting of N, O and S;
each Ar₃ is independently hydrogen, deuterium, a substituted or unsubstituted C₆₋₆₀ aryl, or a substituted or unsubstituted C₂₋₆₀ heteroaryl containing at least one heteroatom selected from the group consisting of N, O and S,
provided that at least one of Ar₁, Ar₂, and Ar₃ is a substituted or unsubstituted C₁₆₋₆₀ aryl polycyclic aromatic ring; and
n1 is an integer of 0 to 7; 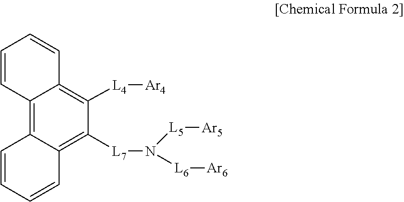
wherein in Chemical Formula 2:
Ar₄ is hydrogen, a substituted or unsubstituted C₆₋₆₀ aryl, or a substituted or unsubstituted C₂₋₆₀ heteroaryl containing at least one heteroatom selected from the group consisting of N, O and S;
Ar₅ and Ar₆ are each a substituted or unsubstituted C₆₋₆₀ aryl or a substituted or unsubstituted C₂₋₆₀ heteroaryl containing at least one heteroatom selected from the group consisting of N, O and S;
L₄ to L₆ are each independently a single bond, a substituted or unsubstituted C₆₋₆₀ arylene, or a substituted or unsubstituted C₂₋₆₀ heteroarylene containing at least one heteroatom selected from the group consisting of N, O and S; and
L₇ is substituted or unsubstituted C₆₋₆₀ arylene,
provided that Ar₃ is not 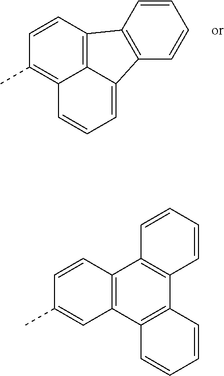
when Ar₃ is a substituted or unsubstituted C₁₆₋₆₀ aryl polycyclic aromatic ring.

2. The organic light emitting device of claim 1, wherein the compound of Chemical Formula 1 is any one of the following Chemical Formula 1-1 to Chemical Formula 1-3: 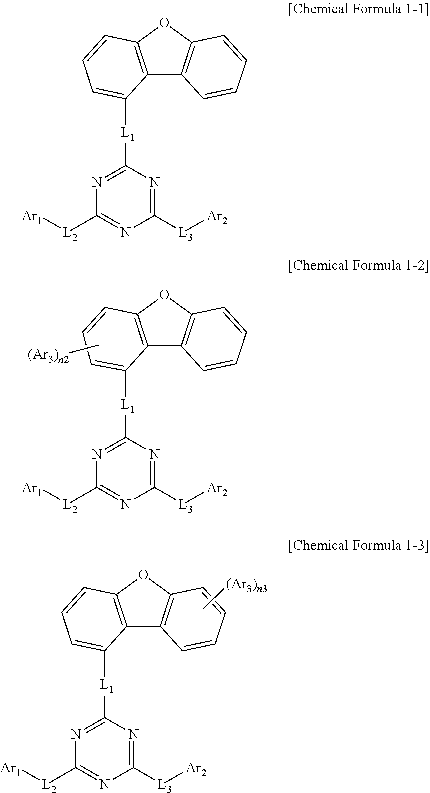
wherein in Chemical Formulae 1-1 to 1-3:
L₁ to L₃ and Ar₁ to Ar₃ are as defined in claim 1;
n2 is an integer of 1 to 3; and
n3 is an integer of 1 to 4.

3. The organic light emitting device of claim 1, wherein L₁ to L₃ are each independently a single bond, phenylene, biphenylene, or naphthylene.

4. The organic light emitting device of claim 1, wherein Ar₁ and Ar₂ are each independently phenyl, phenyl substituted with naphthyl, biphenyl, terphenyl, naphthyl, naphthyl substituted with phenyl, anthracenyl, phenanthrenyl, naphthacenyl, benzanthracenyl, chrysenyl, benzophenanthrenyl, pyrenyl, fluoranthenyl, triphenylenyl, perylenyl, dihydroindenyl, dibenzofuranyl, dibenzothiophenyl, benzonaphthofuranyl, or benzonaphthothiophenyl.

5. The organic light emitting device of claim 1, wherein each Ar₃ is independently hydrogen, deuterium, phenyl, phenyl substituted with naphthyl, biphenyl, terphenyl, naphthyl, naphthyl substituted with phenyl, anthracenyl, phenanthrenyl, naphthacenyl, benzanthracenyl, chrysenyl, benzophenanthrenyl, pyrenyl, fluoranthenyl, triphenylenyl, or perylenyl.

6. The organic light emitting device of claim 1, wherein at least one of Ar₁, Ar₂ and Ar₃ is naphthacenyl, benzanthracenyl, chrysenyl, benzophenanthrenyl, pyrenyl, fluoranthenyl, triphenylenyl, or perylenyl.

7. The organic light emitting device of claim 1, wherein n1 is 0 or 1.

8. The organic light emitting device of claim 1, wherein the compound of Chemical Formula 1 is any one compound selected from the group consisting of the following compounds:

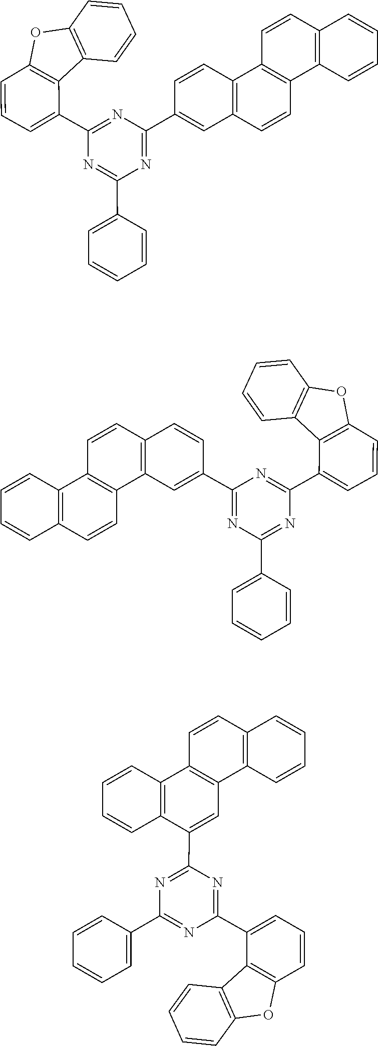 [SMILES: c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cc4c5ccccc5ccc4c4ccccc34)nc(-c3cccc4oc5ccccc5c34)n2)cc1]

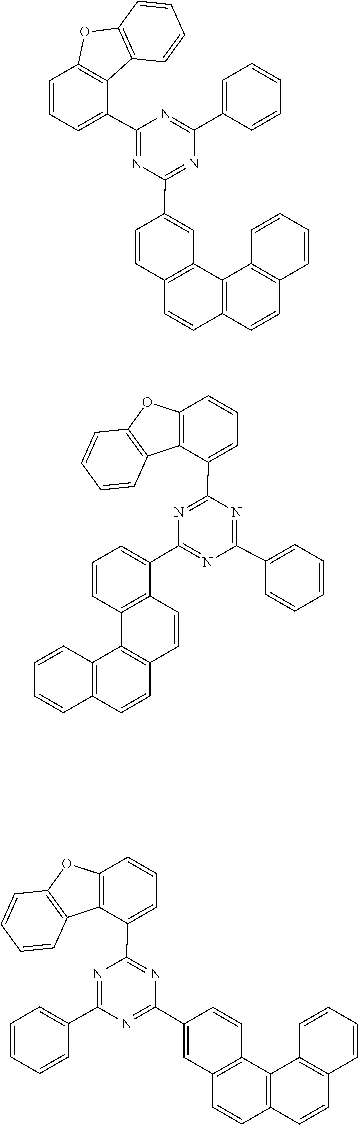 [SMILES: c1ccc(-c2nc(-c3ccc4ccc5ccc6ccccc6c5c4c3)nc(-c3cccc4oc5ccccc5c34)n2)cc1 ; c1ccc(-c2nc(-c3cccc4c3ccc3ccc5ccccc5c34)nc(-c3cccc4oc5ccccc5c34)n2)cc1 ; c1ccc(-c2nc(-c3ccc4c(ccc5ccc6ccccc6c54)c3)nc(-c3cccc4oc5ccccc5c34)n2)cc1]

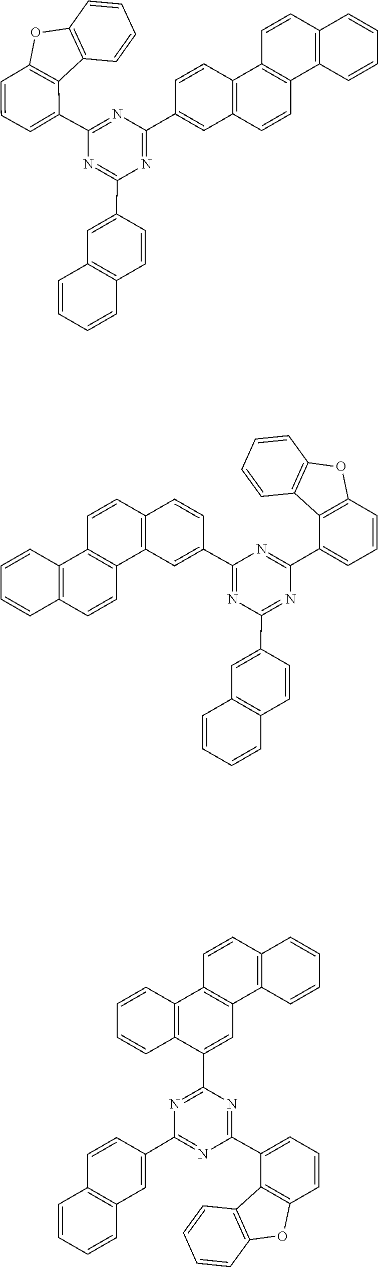 [SMILES: c1ccc2cc(-c3nc(-c4ccc5c(ccc6c7ccccc7ccc56)c4)nc(-c4cccc5oc6ccccc6c45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4cc5c6ccccc6ccc5c5ccccc45)nc(-c4cccc5oc6ccccc6c45)n3)ccc2c1]

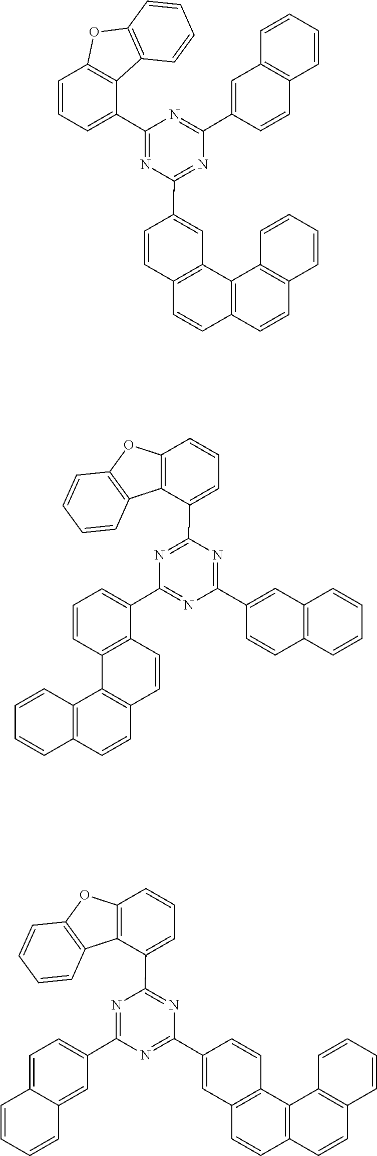 [SMILES: c1ccc2cc(-c3nc(-c4ccc5ccc6ccc7ccccc7c6c5c4)nc(-c4cccc5oc6ccccc6c45)n3)ccc2c1 ; c1ccc2cc(-c3nc(-c4cccc5c4ccc4ccc6ccccc6c45)nc(-c4cccc5oc6ccccc6c45)n3)ccc2c1 ; c1ccc2cc(-c3nc(-c4ccc5c(ccc6ccc7ccccc7c65)c4)nc(-c4cccc5oc6ccccc6c45)n3)ccc2c1]

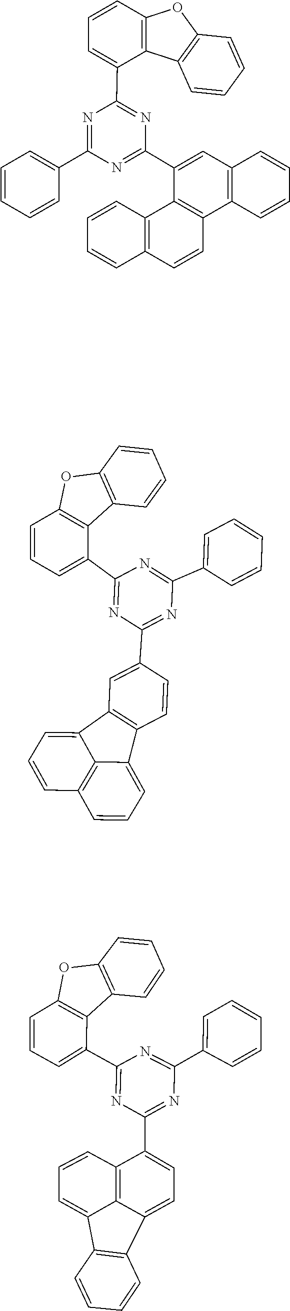 [SMILES: c1ccc(-c2nc(-c3cccc4oc5ccccc5c34)nc(-c3cc4ccccc4c4ccc5ccccc5c34)n2)cc1 ; c1ccc(-c2nc(-c3ccc4c(c3)-c3cccc5cccc-4c35)nc(-c3cccc4oc5ccccc5c34)n2)cc1 ; c1ccc(-c2nc(-c3ccc4c5c(cccc35)-c3ccccc3-4)nc(-c3cccc4oc5ccccc5c34)n2)cc1]

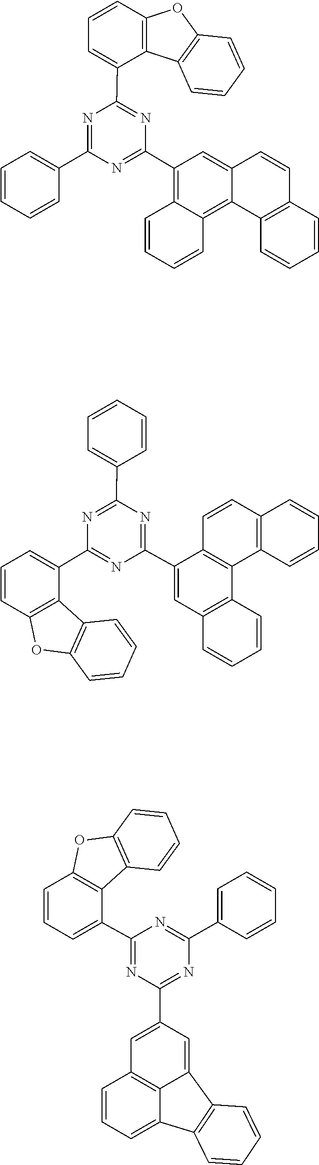 [SMILES: c1ccc(-c2nc(-c3cc4ccc5ccccc5c4c4ccccc34)nc(-c3cccc4oc5ccccc5c34)n2)cc1 ; c1ccc(-c2nc(-c3cc4ccccc4c4c3ccc3ccccc34)nc(-c3cccc4oc5ccccc5c34)n2)cc1 ; c1ccc(-c2nc(-c3cc4c5c(cccc5c3)-c3ccccc3-4)nc(-c3cccc4oc5ccccc5c34)n2)cc1]

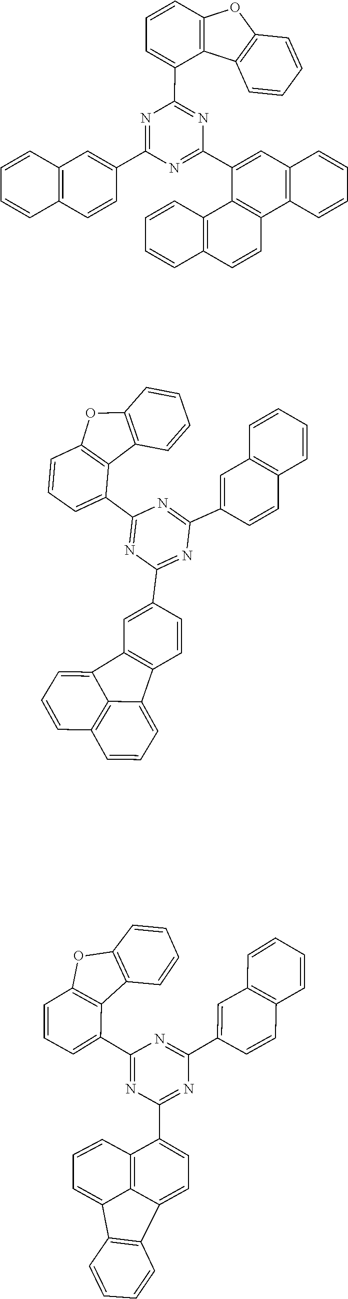 [SMILES: c1ccc2cc(-c3nc(-c4cccc5oc6ccccc6c45)nc(-c4cc5ccccc5c5ccc6ccccc6c45)n3)ccc2c1 ; c1ccc2cc(-c3nc(-c4ccc5c(c4)-c4cccc6cccc-5c46)nc(-c4cccc5oc6ccccc6c45)n3)ccc2c1 ; c1ccc2c(c1)-c1cccc3c(-c4nc(-c5ccc6ccccc6c5)nc(-c5cccc6oc7ccccc7c56)n4)ccc-2c13]

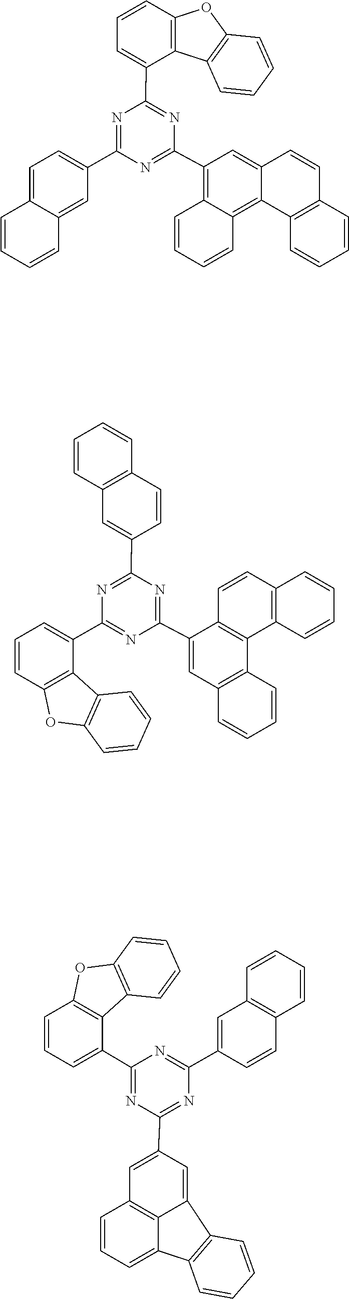 [SMILES: c1ccc2cc(-c3nc(-c4cc5ccc6ccccc6c5c5ccccc45)nc(-c4cccc5oc6ccccc6c45)n3)ccc2c1 ; c1ccc2cc(-c3nc(-c4cc5ccccc5c5c4ccc4ccccc45)nc(-c4cccc5oc6ccccc6c45)n3)ccc2c1 ; c1ccc2c(c1)-c1cccc3cc(-c4nc(-c5ccc6ccccc6c5)nc(-c5cccc6oc7ccccc7c56)n4)cc-2c13]

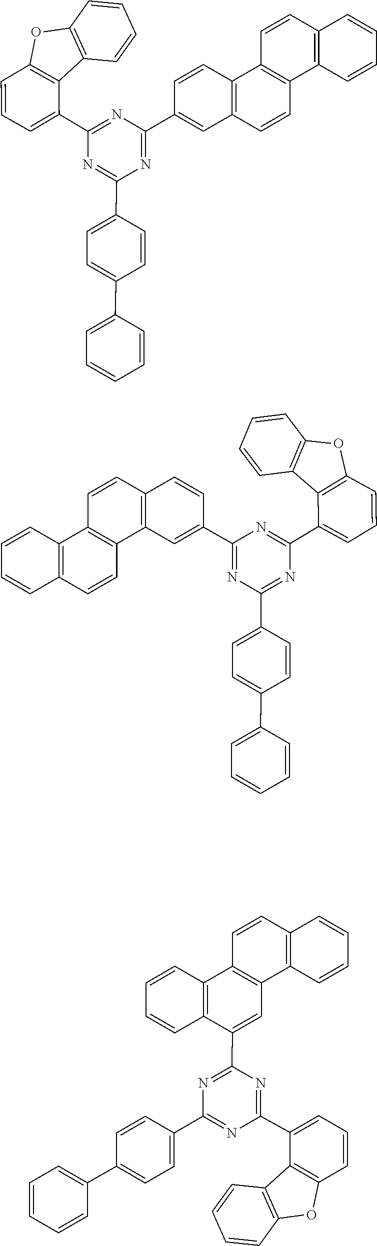 [SMILES: c1ccc(-c2ccc(-c3nc(-c4ccc5c(ccc6c7ccccc7ccc56)c4)nc(-c4cccc5oc6ccccc6c45)n3)cc2)cc1 ; c1ccc(-c2ccc(-c3nc(-c4ccc5ccc6c7ccccc7ccc6c5c4)nc(-c4cccc5oc6ccccc6c45)n3)cc2)cc1 ; c1ccc(-c2ccc(-c3nc(-c4cc5c6ccccc6ccc5c5ccccc45)nc(-c4cccc5oc6ccccc6c45)n3)cc2)cc1]

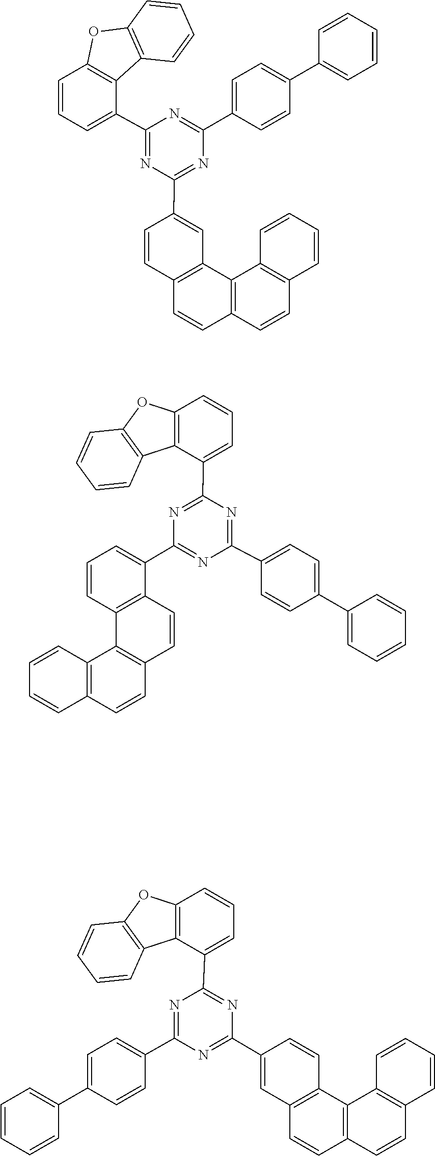 [SMILES: c1ccc(-c2ccc(-c3nc(-c4ccc5ccc6ccc7ccccc7c6c5c4)nc(-c4cccc5oc6ccccc6c45)n3)cc2)cc1 ; c1ccc(-c2ccc(-c3nc(-c4cccc5c4ccc4ccc6ccccc6c45)nc(-c4cccc5oc6ccccc6c45)n3)cc2)cc1 ; c1ccc(-c2ccc(-c3nc(-c4ccc5c(ccc6ccc7ccccc7c65)c4)nc(-c4cccc5oc6ccccc6c45)n3)cc2)cc1]

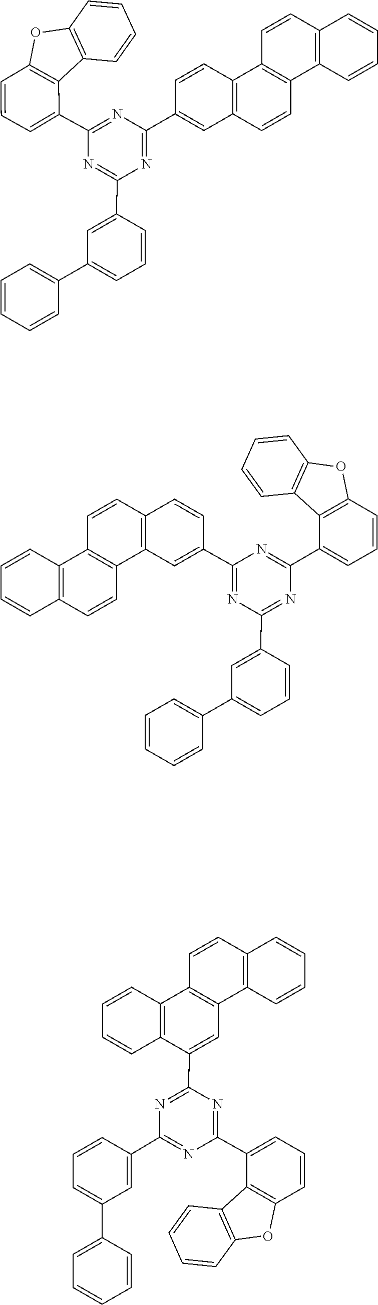 [SMILES: c1ccc(-c2cccc(-c3nc(-c4ccc5c(ccc6c7ccccc7ccc56)c4)nc(-c4cccc5oc6ccccc6c45)n3)c2)cc1 ; c1ccc(-c2cccc(-c3nc(-c4ccc5ccc6c7ccccc7ccc6c5c4)nc(-c4cccc5oc6ccccc6c45)n3)c2)cc1 ; c1ccc(-c2cccc(-c3nc(-c4cc5c6ccccc6ccc5c5ccccc45)nc(-c4cccc5oc6ccccc6c45)n3)c2)cc1]

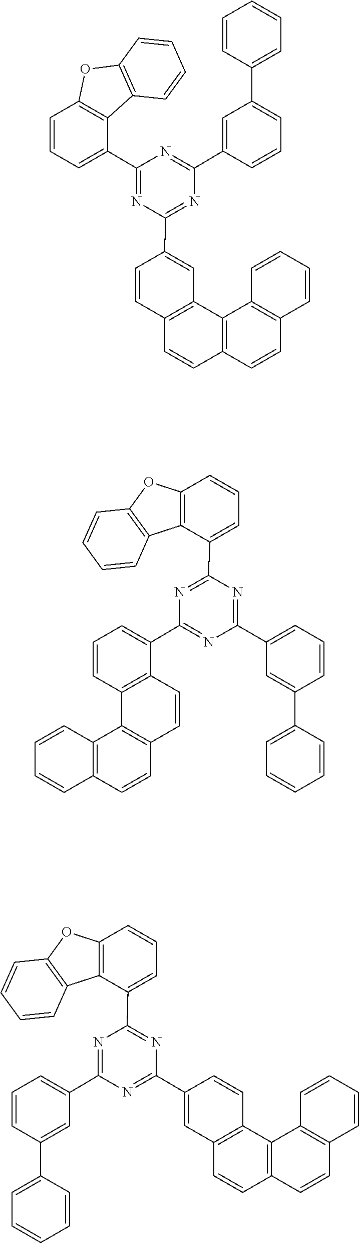 [SMILES: c1ccc(-c2cccc(-c3nc(-c4ccc5ccc6ccc7ccccc7c6c5c4)nc(-c4cccc5oc6ccccc6c45)n3)c2)cc1 ; c1ccc(-c2cccc(-c3nc(-c4cccc5c4ccc4ccc6ccccc6c45)nc(-c4cccc5oc6ccccc6c45)n3)c2)cc1 ; c1ccc(-c2cccc(-c3nc(-c4ccc5c(ccc6ccc7ccccc7c65)c4)nc(-c4cccc5oc6ccccc6c45)n3)c2)cc1]

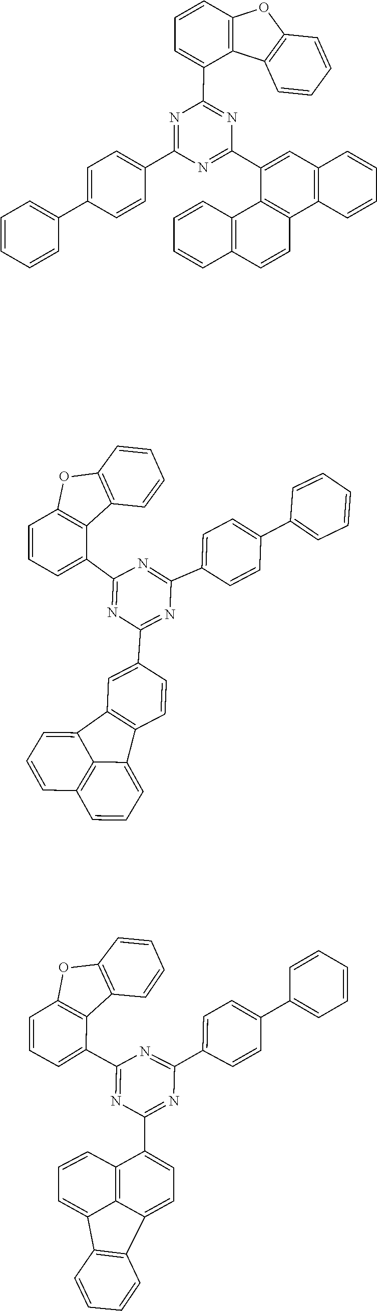 [SMILES: c1ccc(-c2ccc(-c3nc(-c4cccc5oc6ccccc6c45)nc(-c4cc5ccccc5c5ccc6ccccc6c45)n3)cc2)cc1 ; c1ccc(-c2ccc(-c3nc(-c4ccc5c(c4)-c4cccc6cccc-5c46)nc(-c4cccc5oc6ccccc6c45)n3)cc2)cc1 ; c1ccc(-c2ccc(-c3nc(-c4ccc5c6c(cccc46)-c4ccccc4-5)nc(-c4cccc5oc6ccccc6c45)n3)cc2)cc1]

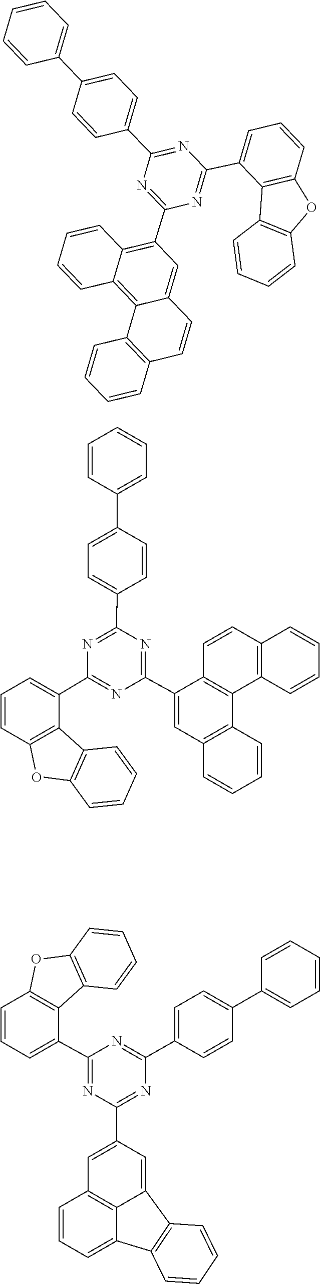

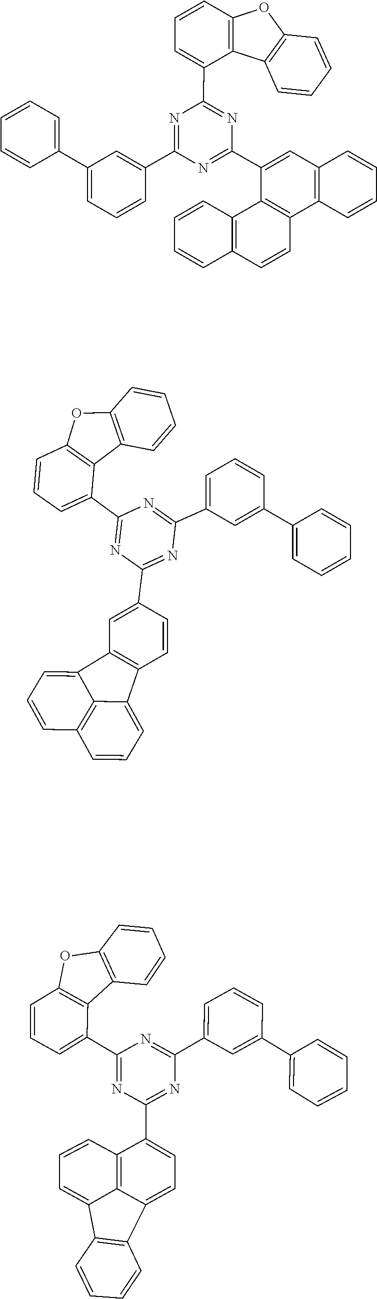 [SMILES: c1ccc(-c2cccc(-c3nc(-c4cccc5oc6ccccc6c45)nc(-c4cc5ccccc5c5ccc6ccccc6c45)n3)c2)cc1 ; c1ccc(-c2cccc(-c3nc(-c4ccc5c(c4)-c4cccc6cccc-5c46)nc(-c4cccc5oc6ccccc6c45)n3)c2)cc1 ; c1ccc(-c2cccc(-c3nc(-c4ccc5c6c(cccc46)-c4ccccc4-5)nc(-c4cccc5oc6ccccc6c45)n3)c2)cc1]

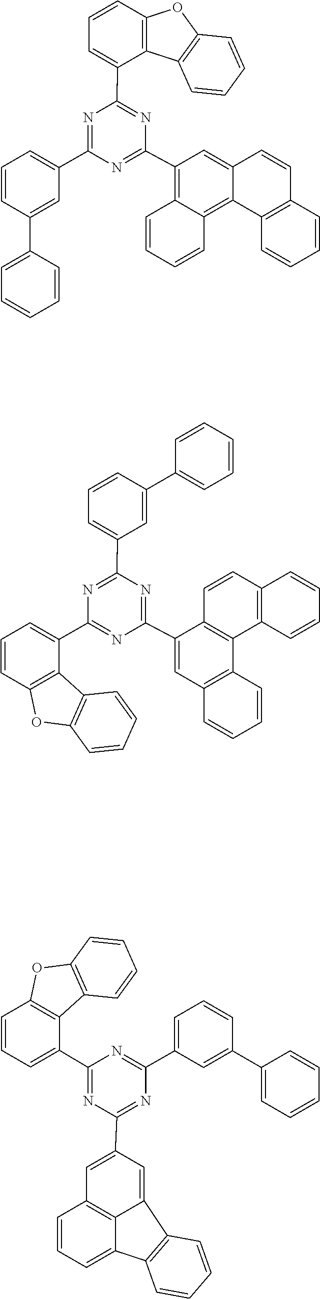 [SMILES: c1ccc(-c2cccc(-c3nc(-c4cc5ccc6ccccc6c5c5ccccc45)nc(-c4cccc5oc6ccccc6c45)n3)c2)cc1 ; c1ccc(-c2cccc(-c3nc(-c4cc5ccccc5c5c4ccc4ccccc45)nc(-c4cccc5oc6ccccc6c45)n3)c2)cc1 ; c1ccc(-c2cccc(-c3nc(-c4cc5c6c(cccc6c4)-c4ccccc4-5)nc(-c4cccc5oc6ccccc6c45)n3)c2)cc1]

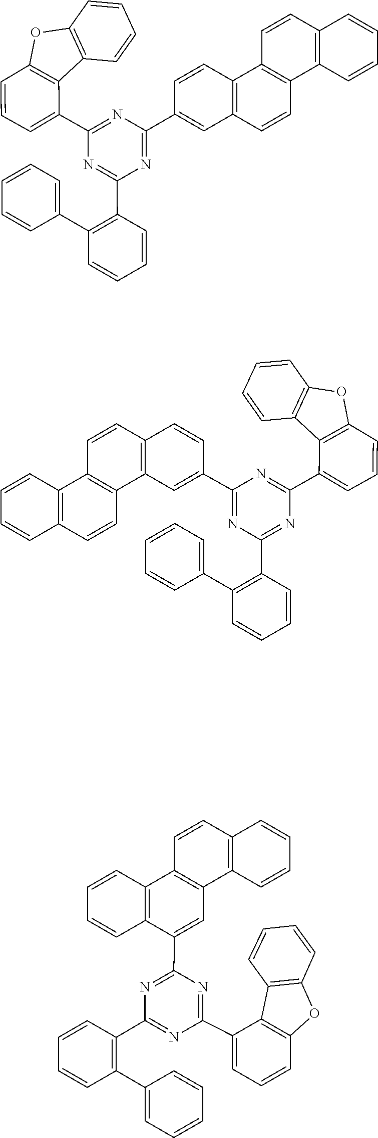 [SMILES: c1ccc(-c2ccccc2-c2nc(-c3ccc4c(ccc5c6ccccc6ccc45)c3)nc(-c3cccc4oc5ccccc5c34)n2)cc1 ; c1ccc(-c2ccccc2-c2nc(-c3ccc4ccc5c6ccccc6ccc5c4c3)nc(-c3cccc4oc5ccccc5c34)n2)cc1 ; c1ccc(-c2ccccc2-c2nc(-c3cc4c5ccccc5ccc4c4ccccc34)nc(-c3cccc4oc5ccccc5c34)n2)cc1]

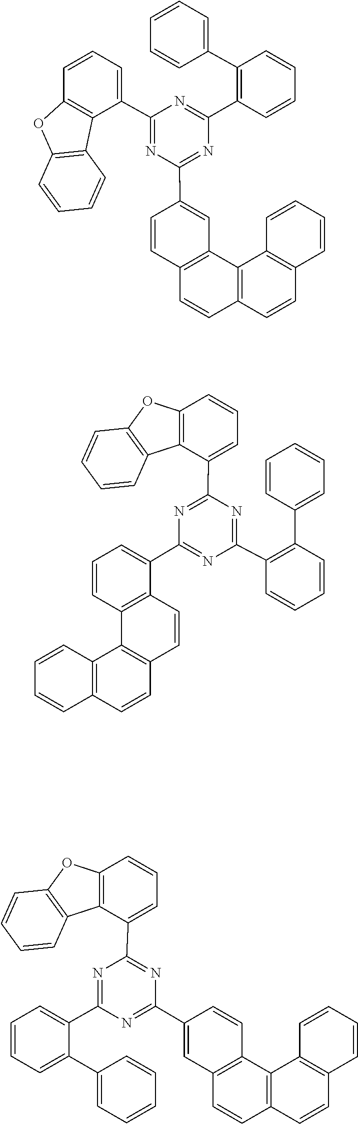 [SMILES: c1ccc(-c2ccccc2-c2nc(-c3ccc4ccc5ccc6ccccc6c5c4c3)nc(-c3cccc4oc5ccccc5c34)n2)cc1 ; c1ccc(-c2ccccc2-c2nc(-c3cccc4c3ccc3ccc5ccccc5c34)nc(-c3cccc4oc5ccccc5c34)n2)cc1 ; c1ccc(-c2ccccc2-c2nc(-c3ccc4c(ccc5ccc6ccccc6c54)c3)nc(-c3cccc4oc5ccccc5c34)n2)cc1]

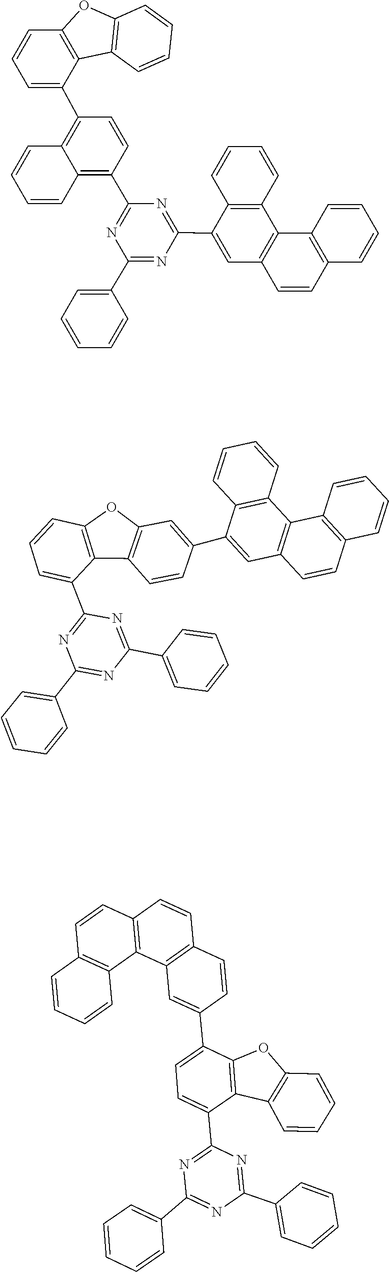 [SMILES: c1ccc(-c2nc(-c3ccc(-c4cccc5oc6ccccc6c45)c4ccccc34)nc(-c3cc4ccc5ccccc5c4c4ccccc34)n2)cc1 ; c1ccc(-c2nc(-c3ccccc3)nc(-c3cccc4oc5cc(-c6cc7ccc8ccccc8c7c7ccccc67)ccc5c34)n2)cc1 ; c1ccc(-c2nc(-c3ccccc3)nc(-c3ccc(-c4ccc5ccc6ccc7ccccc7c6c5c4)c4oc5ccccc5c34)n2)cc1]

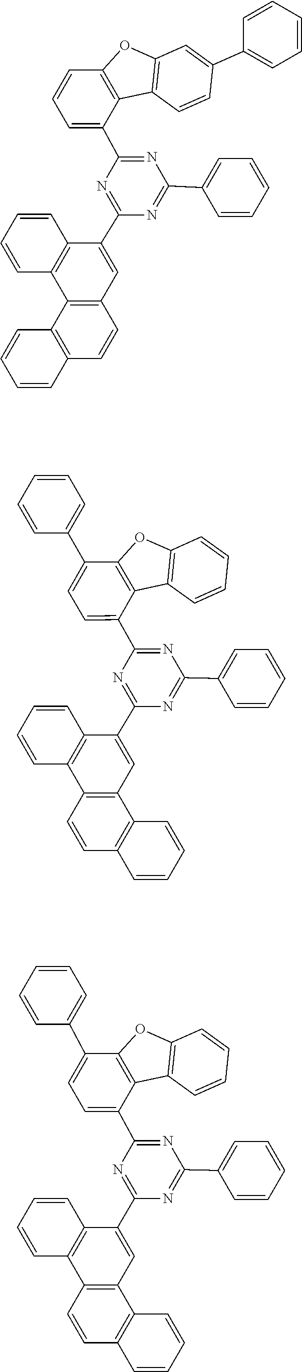 [SMILES: c1ccc(-c2ccc3c(c2)oc2cccc(-c4nc(-c5ccccc5)nc(-c5cc6ccc7ccccc7c6c6ccccc56)n4)c23)cc1 ; c1ccc(-c2nc(-c3cc4c5ccccc5ccc4c4ccccc34)nc(-c3ccc(-c4ccccc4)c4oc5ccccc5c34)n2)cc1 ; c1ccc(-c2nc(-c3cc4c5ccccc5ccc4c4ccccc34)nc(-c3ccc(-c4ccccc4)c4oc5ccccc5c34)n2)cc1]

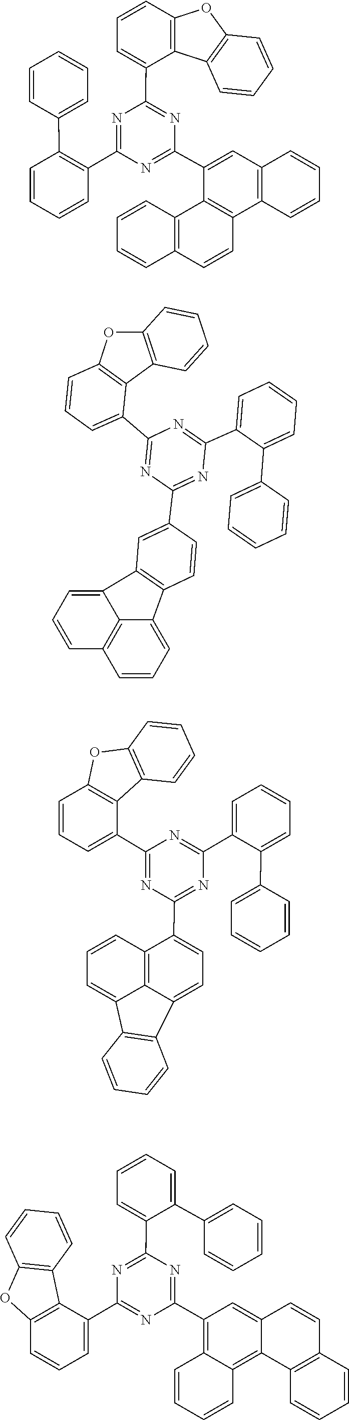

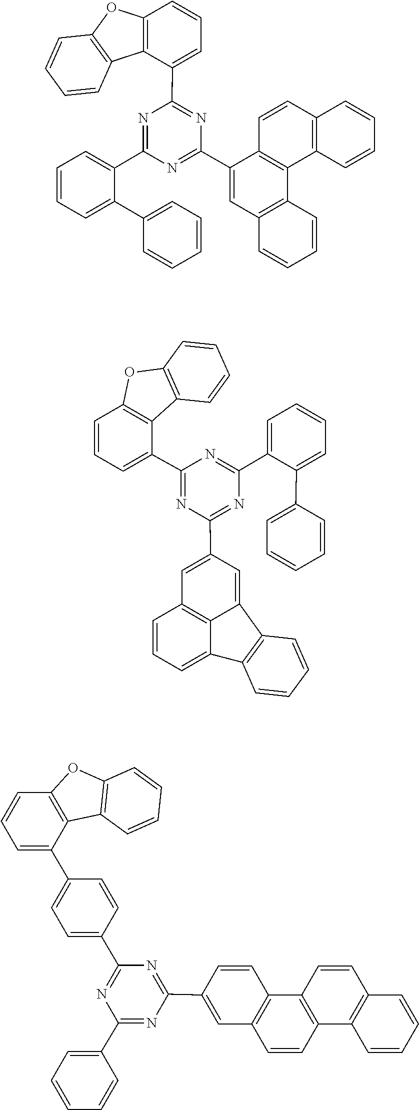 [SMILES: c1ccc(-c2ccccc2-c2nc(-c3cc4ccccc4c4c3ccc3ccccc34)nc(-c3cccc4oc5ccccc5c34)n2)cc1 ; c1ccc(-c2ccccc2-c2nc(-c3cc4c5c(cccc5c3)-c3ccccc3-4)nc(-c3cccc4oc5ccccc5c34)n2)cc1 ; c1ccc(-c2nc(-c3ccc(-c4cccc5oc6ccccc6c45)cc3)nc(-c3ccc4c(ccc5c6ccccc6ccc45)c3)n2)cc1]

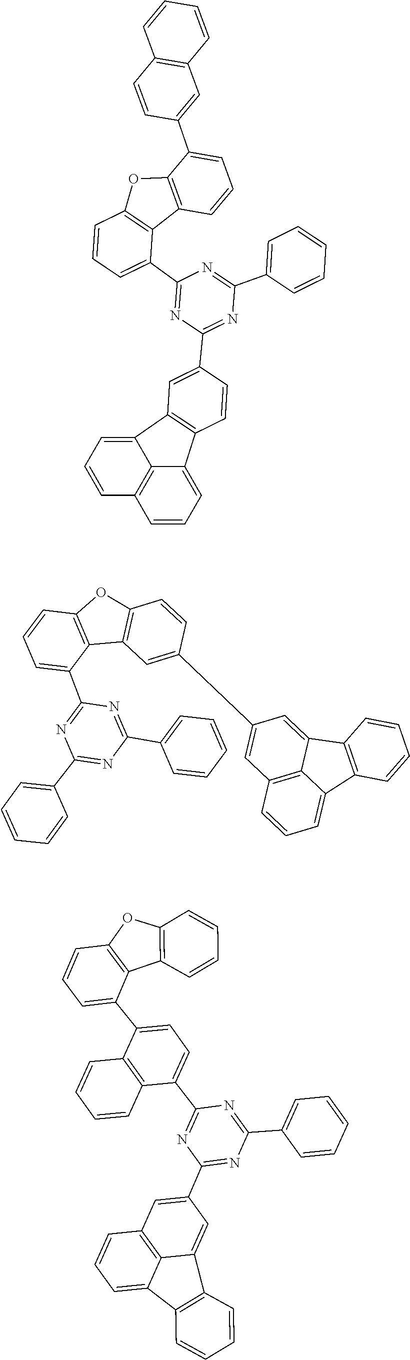 [SMILES: c1ccc(-c2nc(-c3ccc4c(c3)-c3cccc5cccc-4c35)nc(-c3cccc4oc5c(-c6ccc7ccccc7c6)cccc5c34)n2)cc1 ; c1ccc(-c2nc(-c3ccccc3)nc(-c3cccc4oc5ccc(-c6cc7c8c(cccc8c6)-c6ccccc6-7)cc5c34)n2)cc1 ; c1ccc(-c2nc(-c3cc4c5c(cccc5c3)-c3ccccc3-4)nc(-c3ccc(-c4cccc5oc6ccccc6c45)c4ccccc34)n2)cc1]

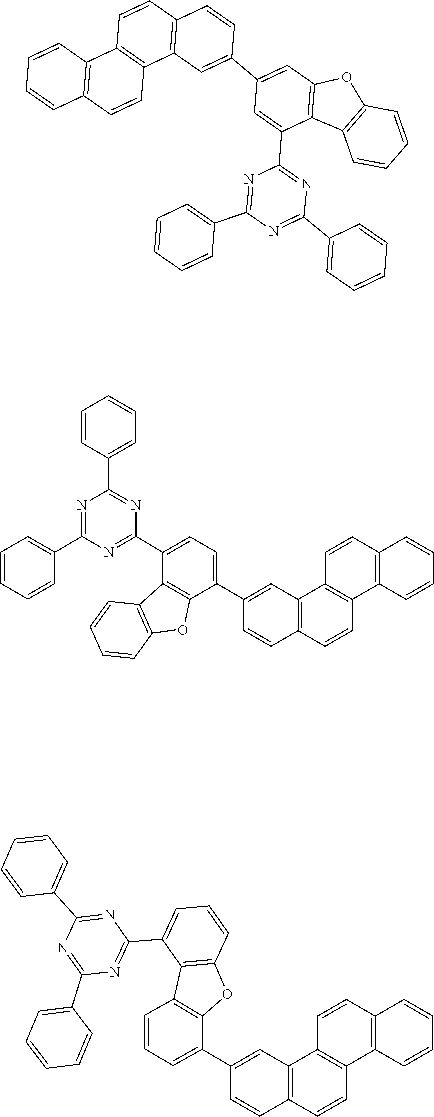 [SMILES: c1ccc(-c2nc(-c3ccccc3)nc(-c3cc(-c4ccc5ccc6c7ccccc7ccc6c5c4)cc4oc5ccccc5c34)n2)cc1 ; c1ccc(-c2nc(-c3ccccc3)nc(-c3ccc(-c4ccc5ccc6c7ccccc7ccc6c5c4)c4oc5ccccc5c34)n2)cc1 ; c1ccc(-c2nc(-c3ccccc3)nc(-c3cccc4oc5c(-c6ccc7ccc8c9ccccc9ccc8c7c6)cccc5c34)n2)cc1]

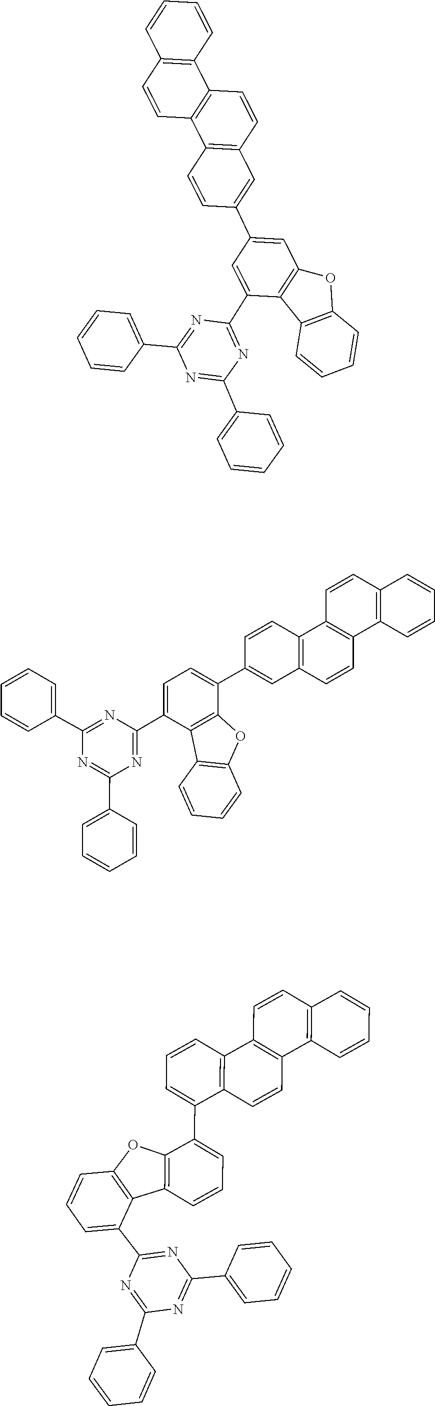 [SMILES: c1ccc(-c2nc(-c3ccccc3)nc(-c3cc(-c4ccc5c(ccc6c7ccccc7ccc56)c4)cc4oc5ccccc5c34)n2)cc1 ; c1ccc(-c2nc(-c3ccccc3)nc(-c3ccc(-c4ccc5c(ccc6c7ccccc7ccc56)c4)c4oc5ccccc5c34)n2)cc1 ; c1ccc(-c2nc(-c3ccccc3)nc(-c3cccc4oc5c(-c6cccc7c6ccc6c8ccccc8ccc76)cccc5c34)n2)cc1]

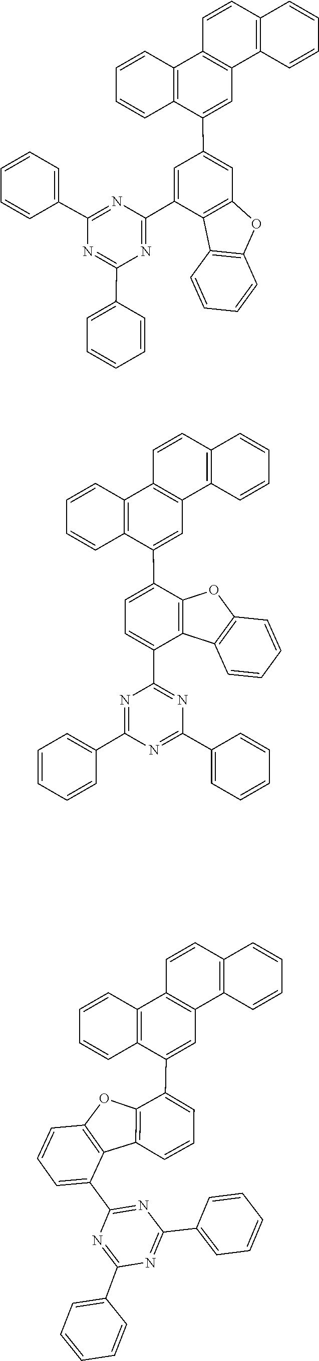 [SMILES: c1ccc(-c2nc(-c3ccccc3)nc(-c3cc(-c4cc5c6ccccc6ccc5c5ccccc45)cc4oc5ccccc5c34)n2)cc1 ; c1ccc(-c2nc(-c3ccccc3)nc(-c3ccc(-c4cc5c6ccccc6ccc5c5ccccc45)c4oc5ccccc5c34)n2)cc1 ; c1ccc(-c2nc(-c3ccccc3)nc(-c3cccc4oc5c(-c6cc7c8ccccc8ccc7c7ccccc67)cccc5c34)n2)cc1]

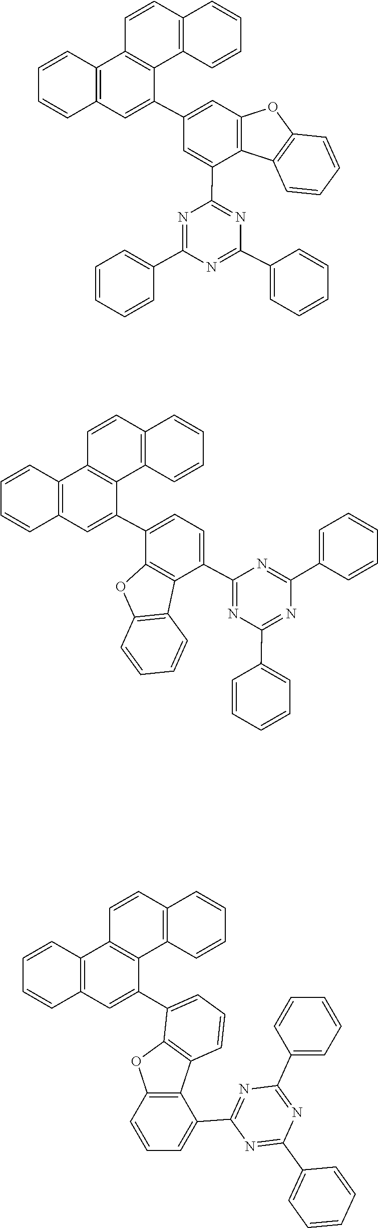 [SMILES: c1ccc(-c2nc(-c3ccccc3)nc(-c3cc(-c4cc5ccccc5c5ccc6ccccc6c45)cc4oc5ccccc5c34)n2)cc1 ; c1ccc(-c2nc(-c3ccccc3)nc(-c3ccc(-c4cc5ccccc5c5ccc6ccccc6c45)c4oc5ccccc5c34)n2)cc1 ; c1ccc(-c2nc(-c3ccccc3)nc(-c3cccc4oc5c(-c6cc7ccccc7c7ccc8ccccc8c67)cccc5c34)n2)cc1]

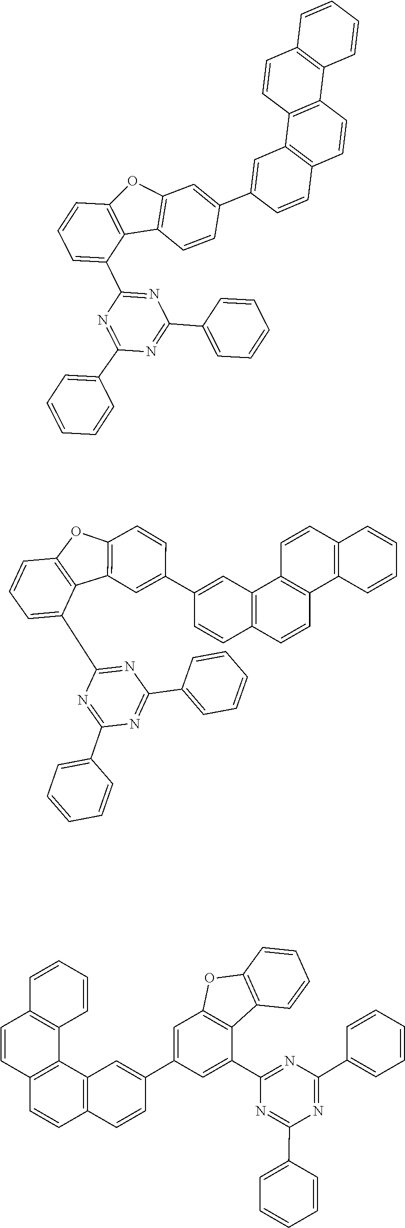 [SMILES: c1ccc(-c2nc(-c3ccccc3)nc(-c3cccc4oc5cc(-c6ccc7ccc8c9ccccc9ccc8c7c6)ccc5c34)n2)cc1 ; c1ccc(-c2nc(-c3ccccc3)nc(-c3cccc4oc5ccc(-c6ccc7ccc8c9ccccc9ccc8c7c6)cc5c34)n2)cc1 ; c1ccc(-c2nc(-c3ccccc3)nc(-c3cc(-c4ccc5ccc6ccc7ccccc7c6c5c4)cc4oc5ccccc5c34)n2)cc1]

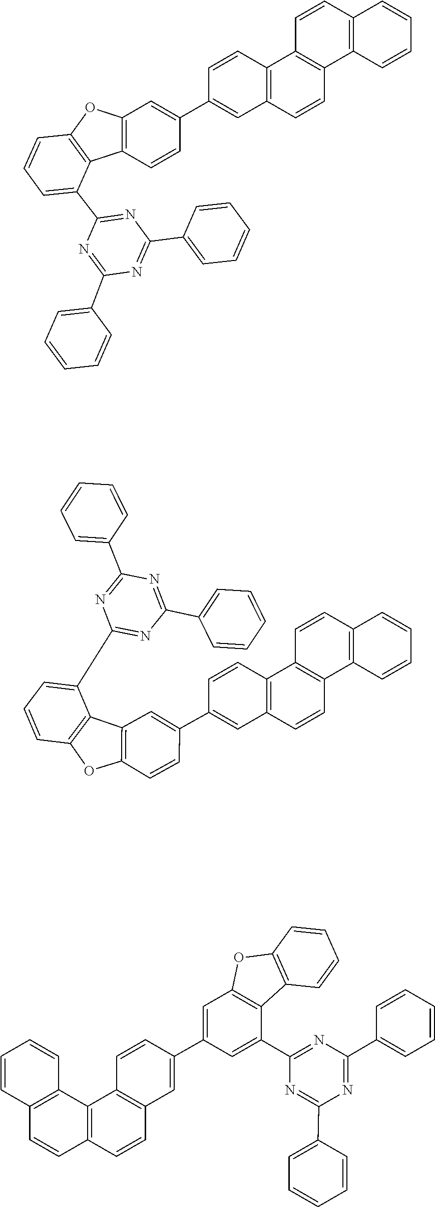 [SMILES: c1ccc(-c2nc(-c3ccccc3)nc(-c3cccc4oc5cc(-c6ccc7c(ccc8c9ccccc9ccc78)c6)ccc5c34)n2)cc1 ; c1ccc(-c2nc(-c3ccccc3)nc(-c3cccc4oc5ccc(-c6ccc7c(ccc8c9ccccc9ccc78)c6)cc5c34)n2)cc1 ; c1ccc(-c2nc(-c3ccccc3)nc(-c3cc(-c4ccc5c(ccc6ccc7ccccc7c65)c4)cc4oc5ccccc5c34)n2)cc1]

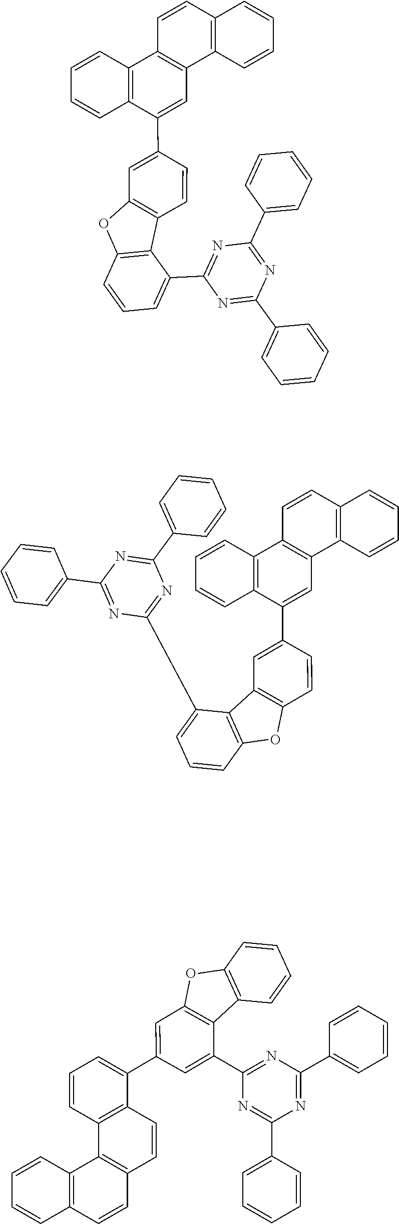 [SMILES: c1ccc(-c2nc(-c3ccccc3)nc(-c3cccc4oc5cc(-c6cc7c8ccccc8ccc7c7ccccc67)ccc5c34)n2)cc1 ; c1ccc(-c2nc(-c3ccccc3)nc(-c3cccc4oc5ccc(-c6cc7c8ccccc8ccc7c7ccccc67)cc5c34)n2)cc1 ; c1ccc(-c2nc(-c3ccccc3)nc(-c3cc(-c4cccc5c4ccc4ccc6ccccc6c45)cc4oc5ccccc5c34)n2)cc1]

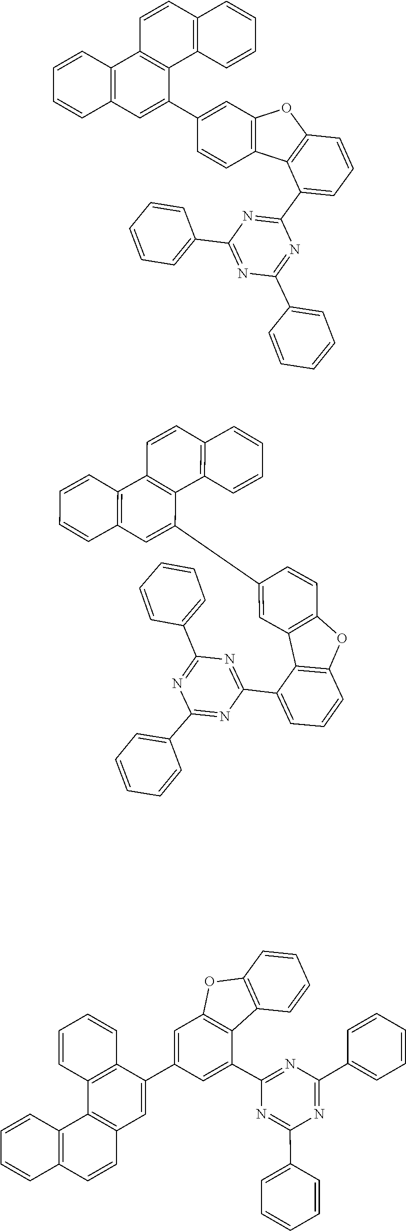 [SMILES: c1ccc(-c2nc(-c3ccccc3)nc(-c3cccc4oc5cc(-c6cc7ccccc7c7ccc8ccccc8c67)ccc5c34)n2)cc1 ; c1ccc(-c2nc(-c3ccccc3)nc(-c3cccc4oc5ccc(-c6cc7ccccc7c7ccc8ccccc8c67)cc5c34)n2)cc1 ; c1ccc(-c2nc(-c3ccccc3)nc(-c3cc(-c4cc5ccc6ccccc6c5c5ccccc45)cc4oc5ccccc5c34)n2)cc1]

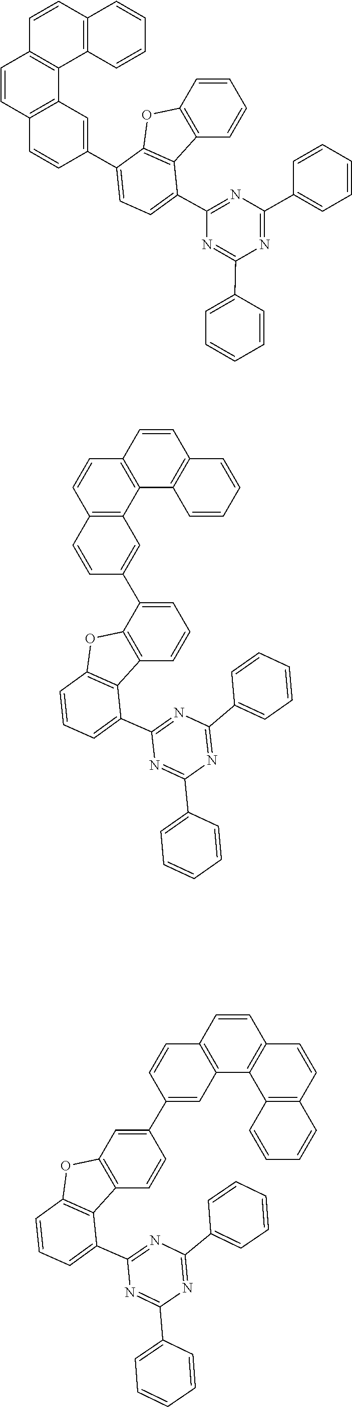 [SMILES: c1ccc(-c2nc(-c3ccccc3)nc(-c3ccc(-c4ccc5ccc6ccc7ccccc7c6c5c4)c4oc5ccccc5c34)n2)cc1 ; c1ccc(-c2nc(-c3ccccc3)nc(-c3cccc4oc5c(-c6ccc7ccc8ccc9ccccc9c8c7c6)cccc5c34)n2)cc1 ; c1ccc(-c2nc(-c3ccccc3)nc(-c3cccc4oc5cc(-c6ccc7ccc8ccc9ccccc9c8c7c6)ccc5c34)n2)cc1]

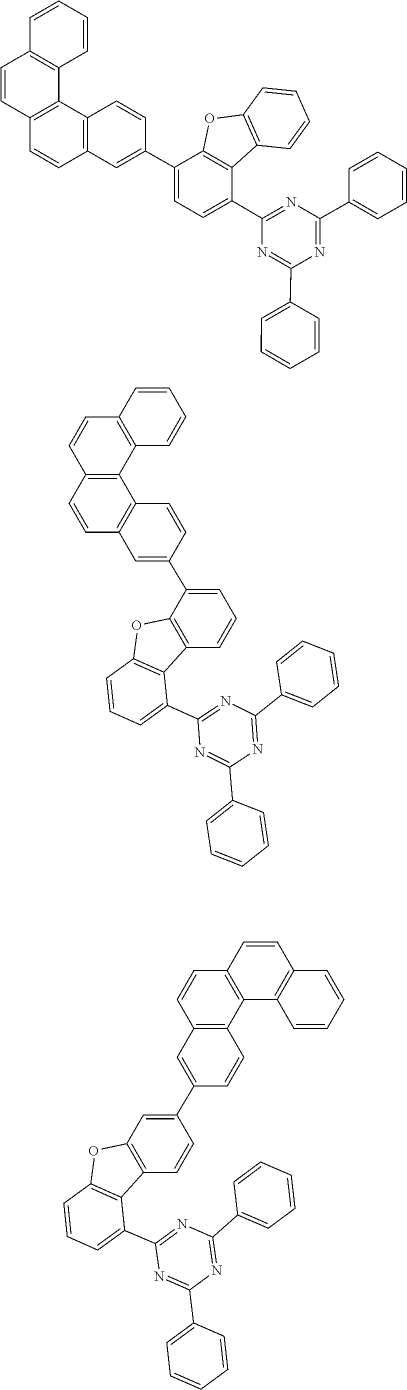 [SMILES: c1ccc(-c2nc(-c3ccccc3)nc(-c3ccc(-c4ccc5c(ccc6ccc7ccccc7c65)c4)c4oc5ccccc5c34)n2)cc1 ; c1ccc(-c2nc(-c3ccccc3)nc(-c3cccc4oc5c(-c6ccc7c(ccc8ccc9ccccc9c87)c6)cccc5c34)n2)cc1 ; c1ccc(-c2nc(-c3ccccc3)nc(-c3cccc4oc5cc(-c6ccc7c(ccc8ccc9ccccc9c87)c6)ccc5c34)n2)cc1]

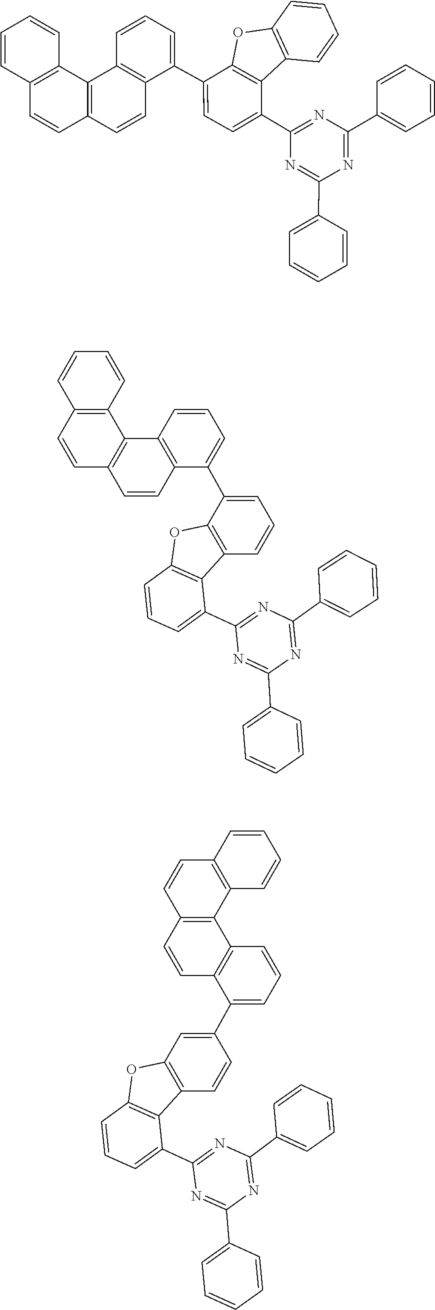 [SMILES: c1ccc(-c2nc(-c3ccccc3)nc(-c3ccc(-c4cccc5c4ccc4ccc6ccccc6c45)c4oc5ccccc5c34)n2)cc1 ; c1ccc(-c2nc(-c3ccccc3)nc(-c3cccc4oc5c(-c6cccc7c6ccc6ccc8ccccc8c67)cccc5c34)n2)cc1 ; c1ccc(-c2nc(-c3ccccc3)nc(-c3cccc4oc5cc(-c6cccc7c6ccc6ccc8ccccc8c67)ccc5c34)n2)cc1]

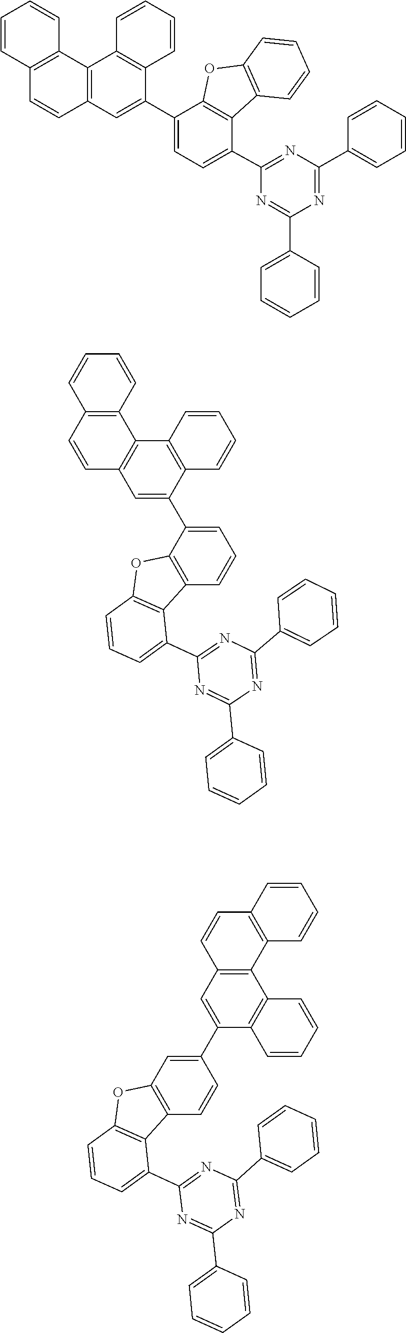 [SMILES: c1ccc(-c2nc(-c3ccccc3)nc(-c3ccc(-c4cc5ccc6ccccc6c5c5ccccc45)c4oc5ccccc5c34)n2)cc1 ; c1ccc(-c2nc(-c3ccccc3)nc(-c3cccc4oc5c(-c6cc7ccc8ccccc8c7c7ccccc67)cccc5c34)n2)cc1 ; c1ccc(-c2nc(-c3ccccc3)nc(-c3cccc4oc5cc(-c6cc7ccc8ccccc8c7c7ccccc67)ccc5c34)n2)cc1]

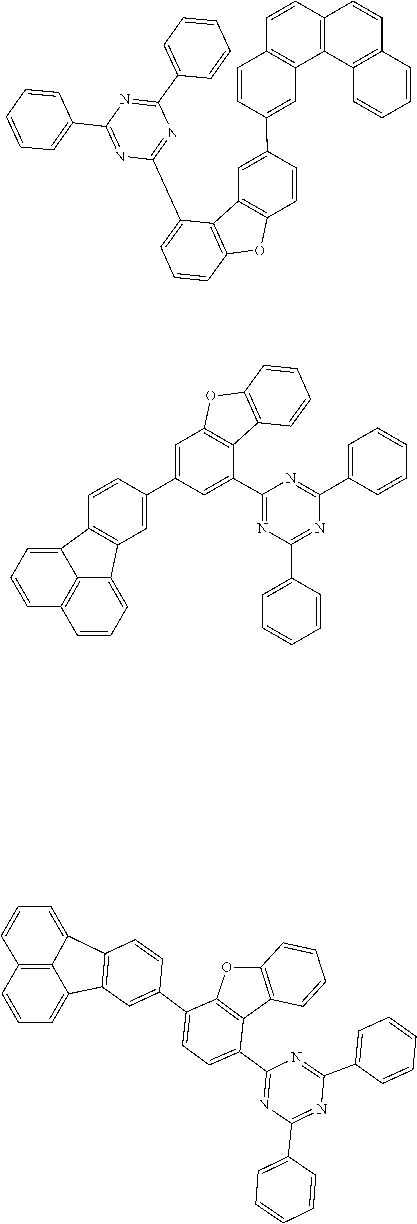 [SMILES: c1ccc(-c2nc(-c3ccccc3)nc(-c3cccc4oc5ccc(-c6ccc7ccc8ccc9ccccc9c8c7c6)cc5c34)n2)cc1 ; c1ccc(-c2nc(-c3ccccc3)nc(-c3cc(-c4ccc5c(c4)-c4cccc6cccc-5c46)cc4oc5ccccc5c34)n2)cc1 ; c1ccc(-c2nc(-c3ccccc3)nc(-c3ccc(-c4ccc5c(c4)-c4cccc6cccc-5c46)c4oc5ccccc5c34)n2)cc1]

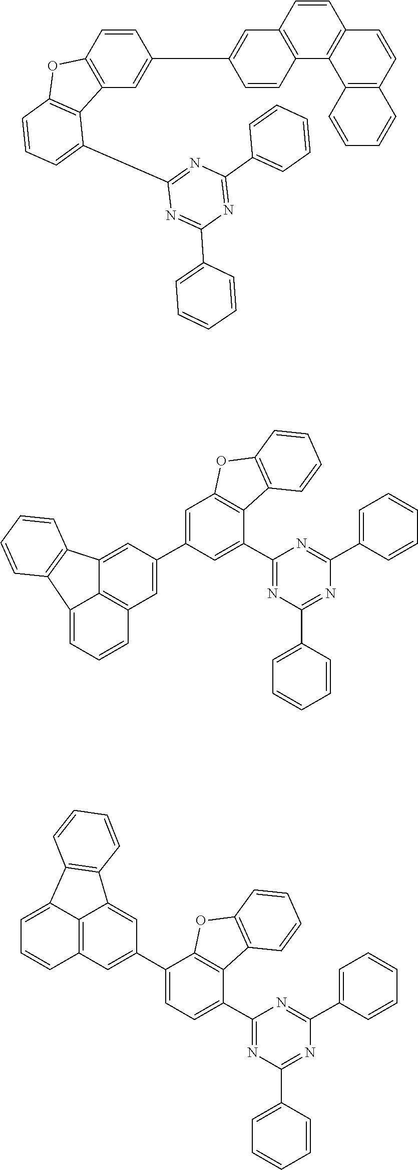 [SMILES: c1ccc(-c2nc(-c3ccccc3)nc(-c3cccc4oc5ccc(-c6ccc7c(ccc8ccc9ccccc9c87)c6)cc5c34)n2)cc1 ; c1ccc(-c2nc(-c3ccccc3)nc(-c3cc(-c4cc5c6c(cccc6c4)-c4ccccc4-5)cc4oc5ccccc5c34)n2)cc1 ; c1ccc(-c2nc(-c3ccccc3)nc(-c3ccc(-c4cc5c6c(cccc6c4)-c4ccccc4-5)c4oc5ccccc5c34)n2)cc1]

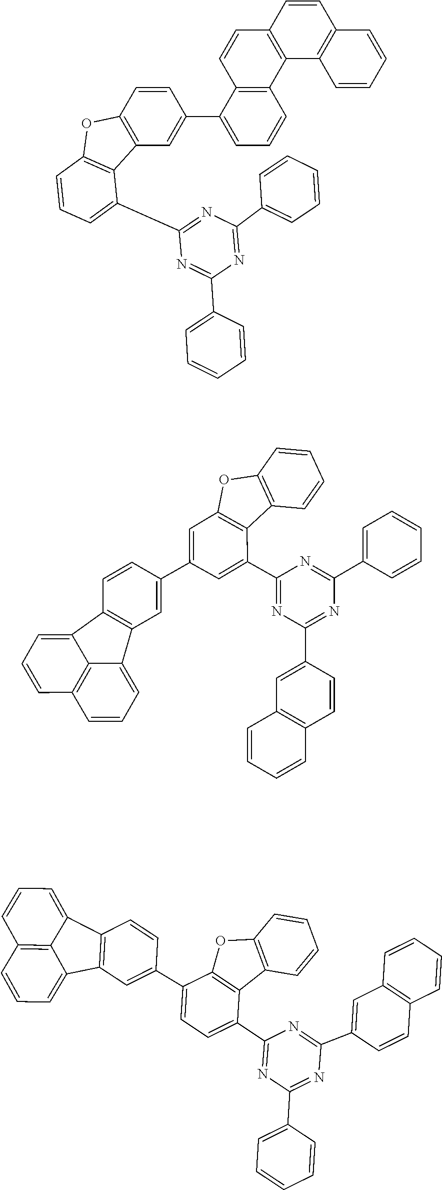 [SMILES: c1ccc(-c2nc(-c3ccccc3)nc(-c3cccc4oc5ccc(-c6cccc7c6ccc6ccc8ccccc8c67)cc5c34)n2)cc1 ; c1ccc(-c2nc(-c3ccc4ccccc4c3)nc(-c3cc(-c4ccc5c(c4)-c4cccc6cccc-5c46)cc4oc5ccccc5c34)n2)cc1 ; c1ccc(-c2nc(-c3ccc4ccccc4c3)nc(-c3ccc(-c4ccc5c(c4)-c4cccc6cccc-5c46)c4oc5ccccc5c34)n2)cc1]

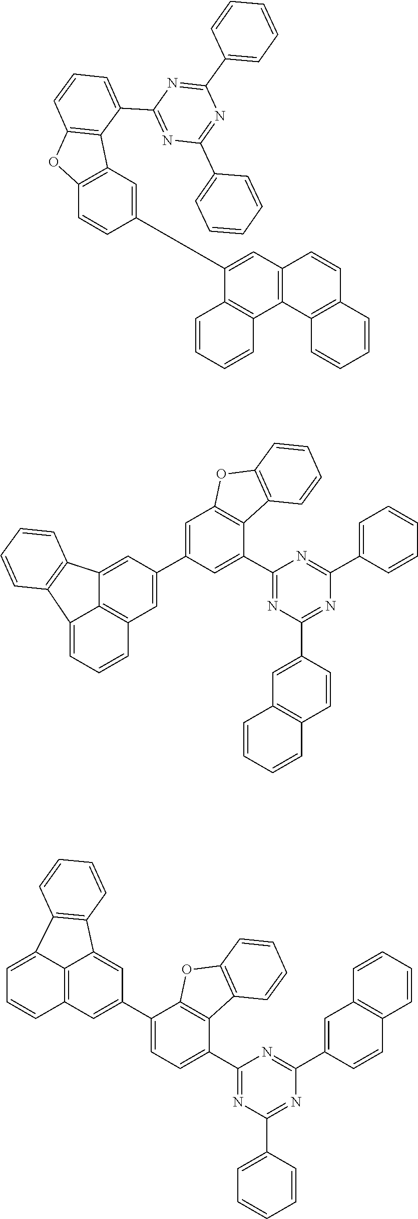 [SMILES: c1ccc(-c2nc(-c3ccccc3)nc(-c3cccc4oc5ccc(-c6cc7ccc8ccccc8c7c7ccccc67)cc5c34)n2)cc1 ; c1ccc(-c2nc(-c3ccc4ccccc4c3)nc(-c3cc(-c4cc5c6c(cccc6c4)-c4ccccc4-5)cc4oc5ccccc5c34)n2)cc1 ; c1ccc(-c2nc(-c3ccc4ccccc4c3)nc(-c3ccc(-c4cc5c6c(cccc6c4)-c4ccccc4-5)c4oc5ccccc5c34)n2)cc1]

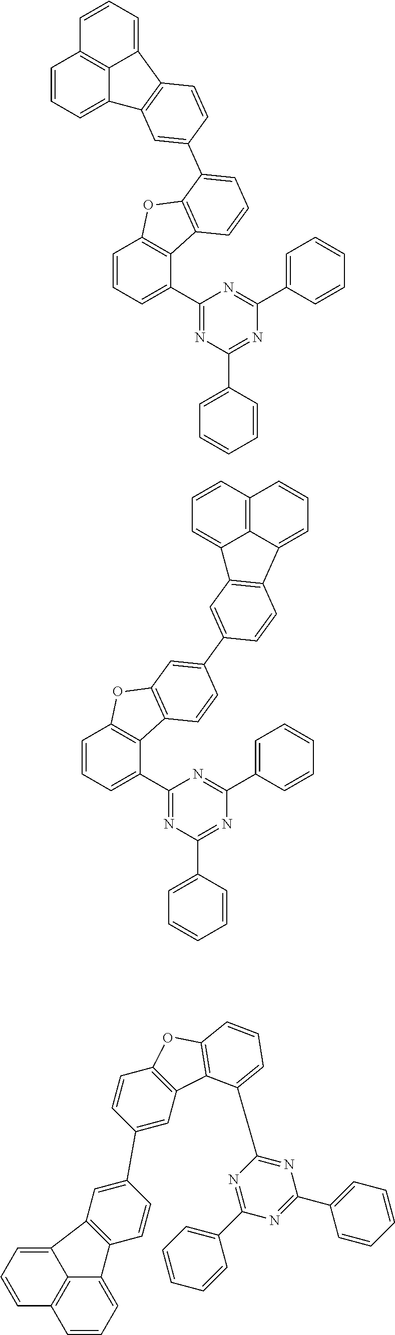

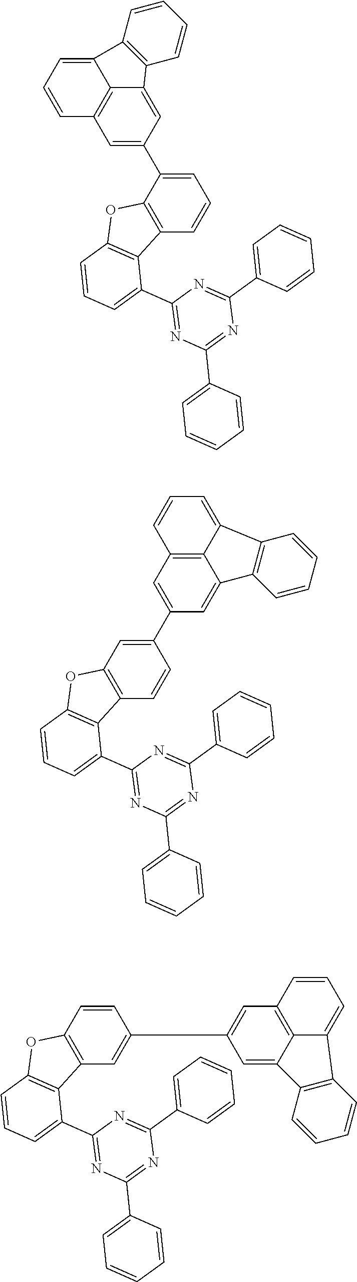 [SMILES: c1ccc(-c2nc(-c3ccccc3)nc(-c3cccc4oc5c(-c6cc7c8c(cccc8c6)-c6ccccc6-7)cccc5c34)n2)cc1 ; c1ccc(-c2nc(-c3ccccc3)nc(-c3cccc4oc5cc(-c6cc7c8c(cccc8c6)-c6ccccc6-7)ccc5c34)n2)cc1 ; c1ccc(-c2nc(-c3ccccc3)nc(-c3cccc4oc5ccc(-c6cc7c8c(cccc8c6)-c6ccccc6-7)cc5c34)n2)cc1]

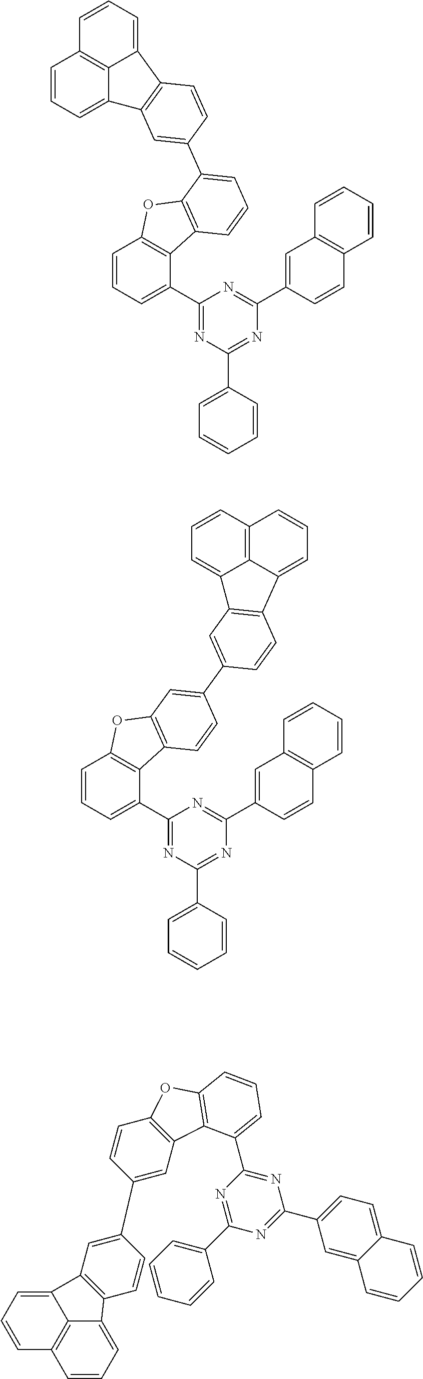 [SMILES: c1ccc(-c2nc(-c3ccc4ccccc4c3)nc(-c3cccc4oc5c(-c6ccc7c(c6)-c6cccc8cccc-7c68)cccc5c34)n2)cc1 ; c1ccc(-c2nc(-c3ccc4ccccc4c3)nc(-c3cccc4oc5cc(-c6ccc7c(c6)-c6cccc8cccc-7c68)ccc5c34)n2)cc1 ; c1ccc(-c2nc(-c3ccc4ccccc4c3)nc(-c3cccc4oc5ccc(-c6ccc7c(c6)-c6cccc8cccc-7c68)cc5c34)n2)cc1]

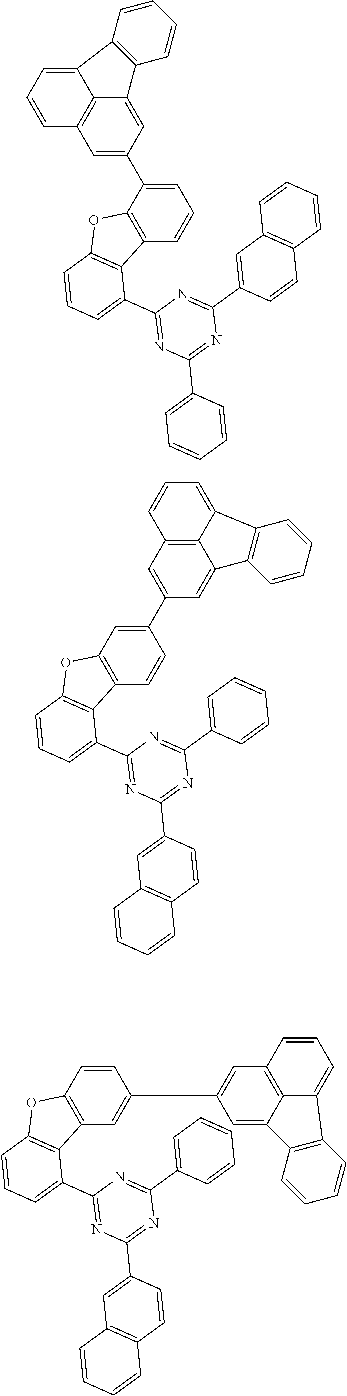

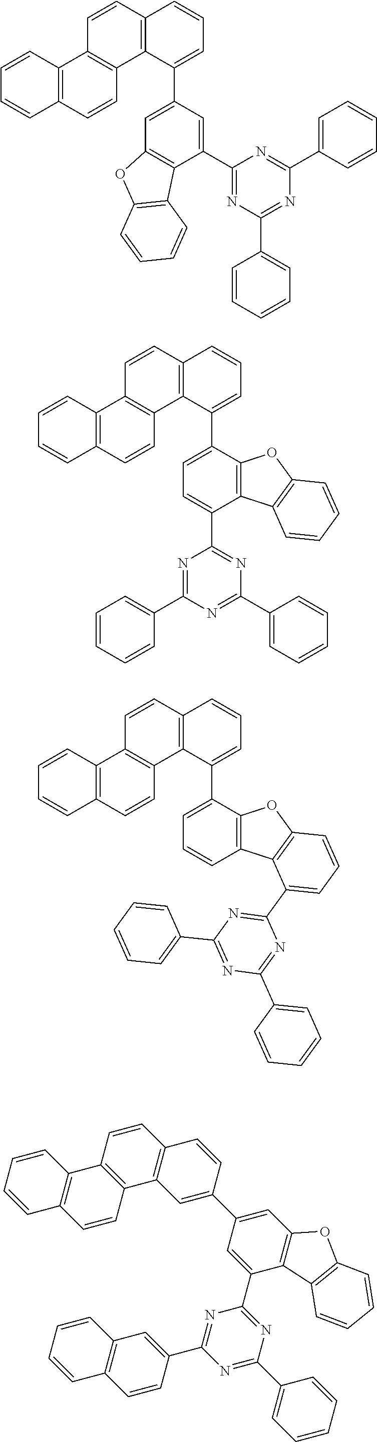

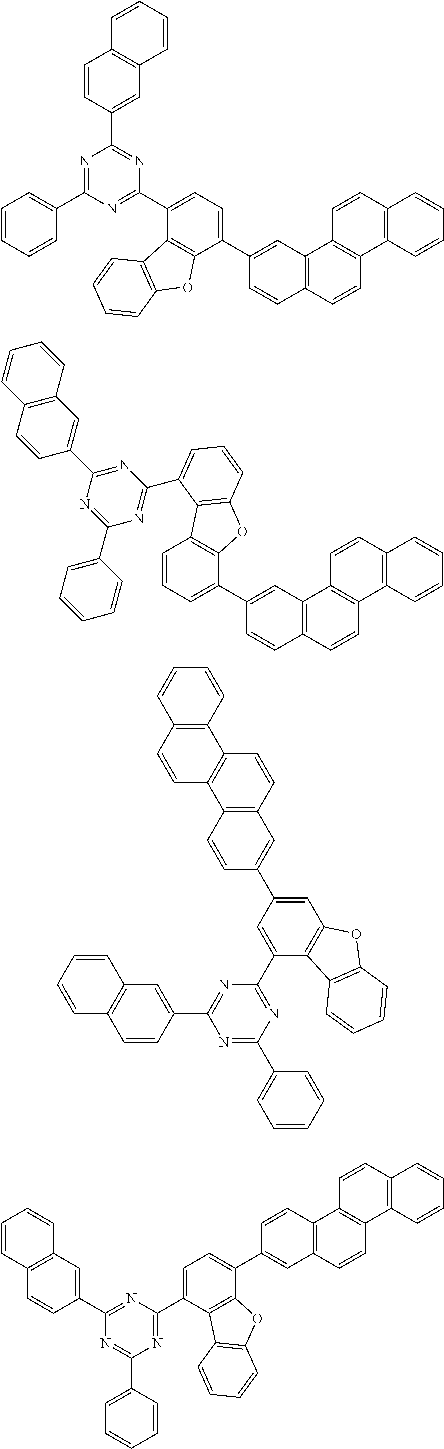 [SMILES: c1ccc(-c2nc(-c3ccc4ccccc4c3)nc(-c3ccc(-c4ccc5ccc6c7ccccc7ccc6c5c4)c4oc5ccccc5c34)n2)cc1 ; c1ccc(-c2nc(-c3ccc4ccccc4c3)nc(-c3cccc4oc5c(-c6ccc7ccc8c9ccccc9ccc8c7c6)cccc5c34)n2)cc1 ; c1ccc(-c2nc(-c3ccc4ccccc4c3)nc(-c3cc(-c4ccc5c(ccc6c7ccccc7ccc56)c4)cc4oc5ccccc5c34)n2)cc1 ; c1ccc(-c2nc(-c3ccc4ccccc4c3)nc(-c3ccc(-c4ccc5c(ccc6c7ccccc7ccc56)c4)c4oc5ccccc5c34)n2)cc1]

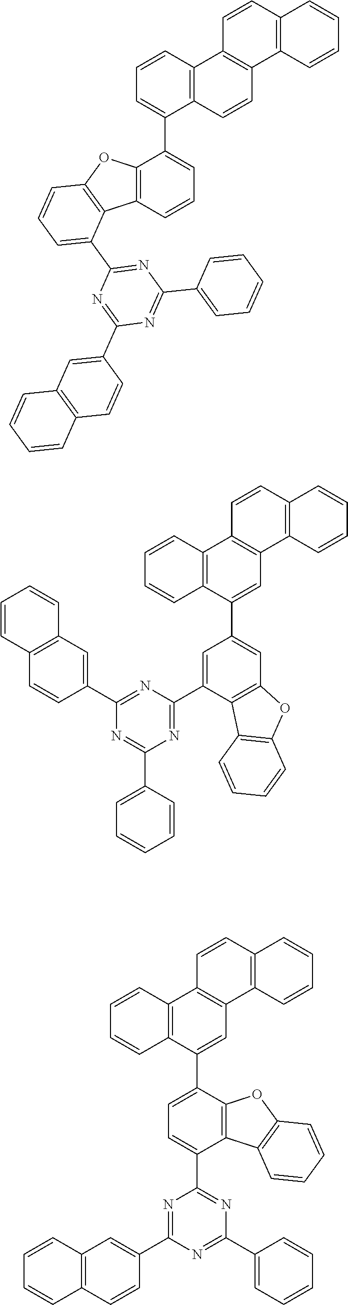 [SMILES: c1ccc(-c2nc(-c3ccc4ccccc4c3)nc(-c3cccc4oc5c(-c6cccc7c6ccc6c8ccccc8ccc76)cccc5c34)n2)cc1 ; c1ccc(-c2nc(-c3ccc4ccccc4c3)nc(-c3cc(-c4cc5c6ccccc6ccc5c5ccccc45)cc4oc5ccccc5c34)n2)cc1 ; c1ccc(-c2nc(-c3ccc4ccccc4c3)nc(-c3ccc(-c4cc5c6ccccc6ccc5c5ccccc45)c4oc5ccccc5c34)n2)cc1]

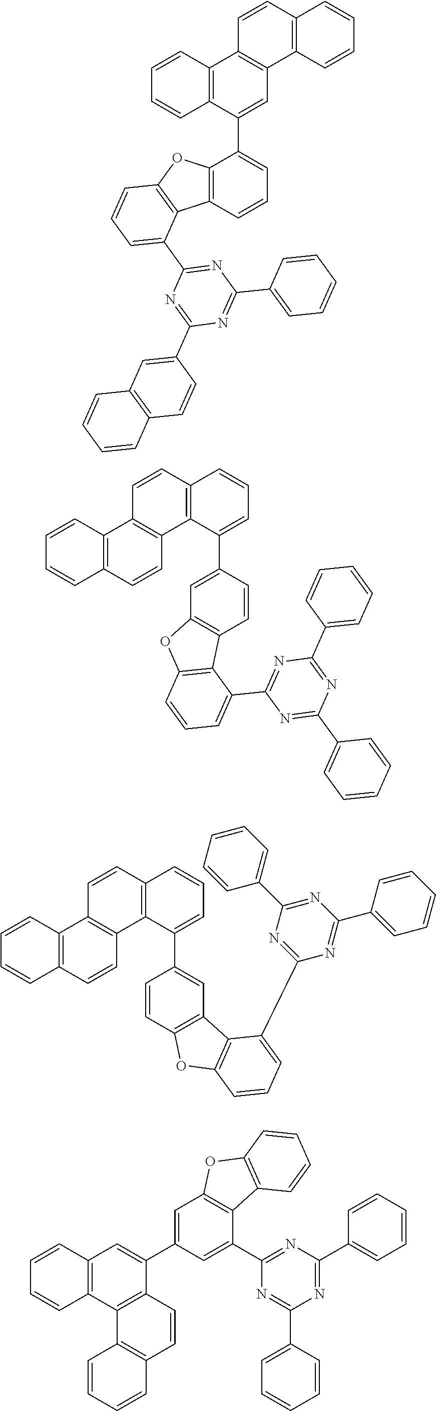

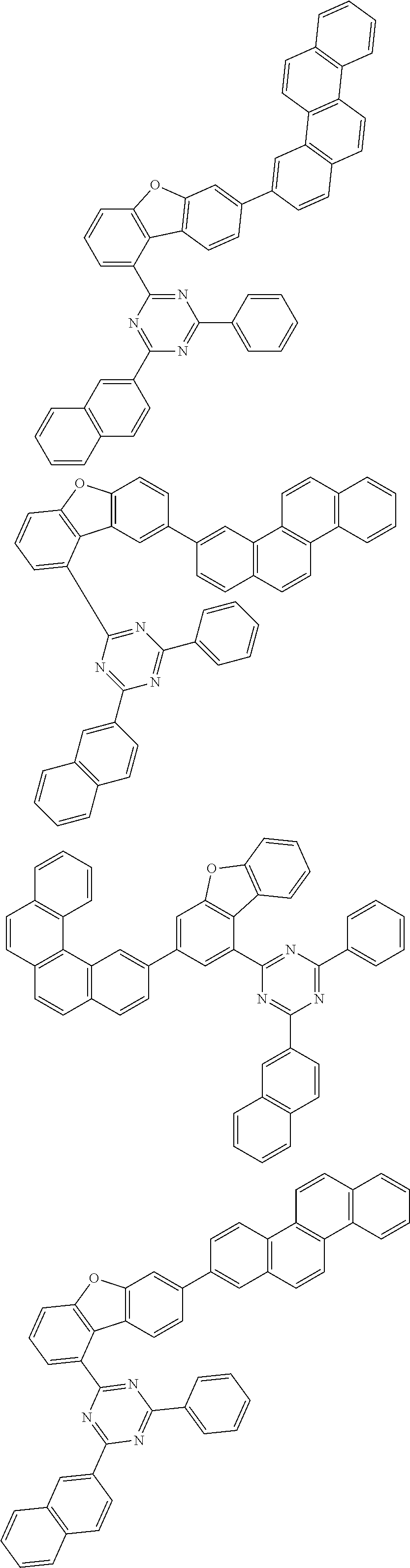

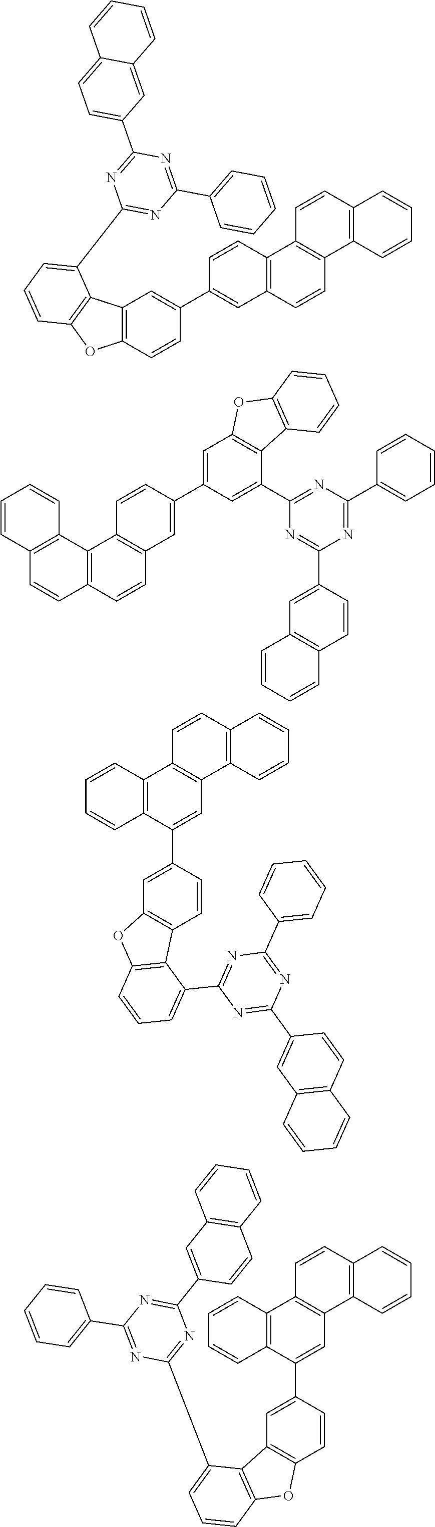

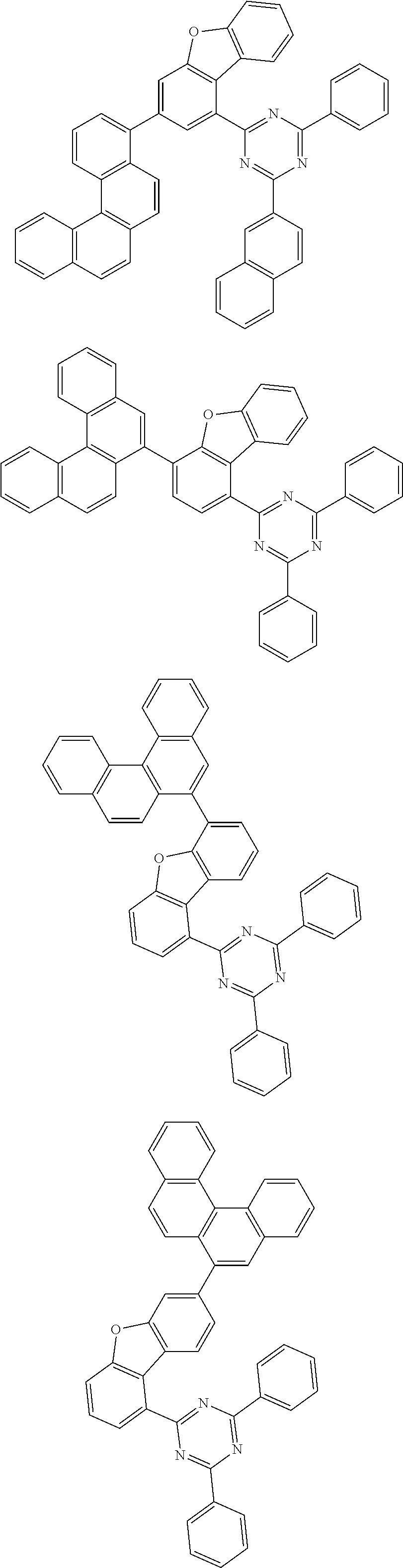 [SMILES: c1ccc(-c2nc(-c3ccc4ccccc4c3)nc(-c3cc(-c4cccc5c4ccc4ccc6ccccc6c45)cc4oc5ccccc5c34)n2)cc1 ; c1ccc(-c2nc(-c3ccccc3)nc(-c3ccc(-c4cc5ccccc5c5c4ccc4ccccc45)c4oc5ccccc5c34)n2)cc1 ; c1ccc(-c2nc(-c3ccccc3)nc(-c3cccc4oc5c(-c6cc7ccccc7c7c6ccc6ccccc67)cccc5c34)n2)cc1 ; c1ccc(-c2nc(-c3ccccc3)nc(-c3cccc4oc5cc(-c6cc7ccccc7c7c6ccc6ccccc67)ccc5c34)n2)cc1]

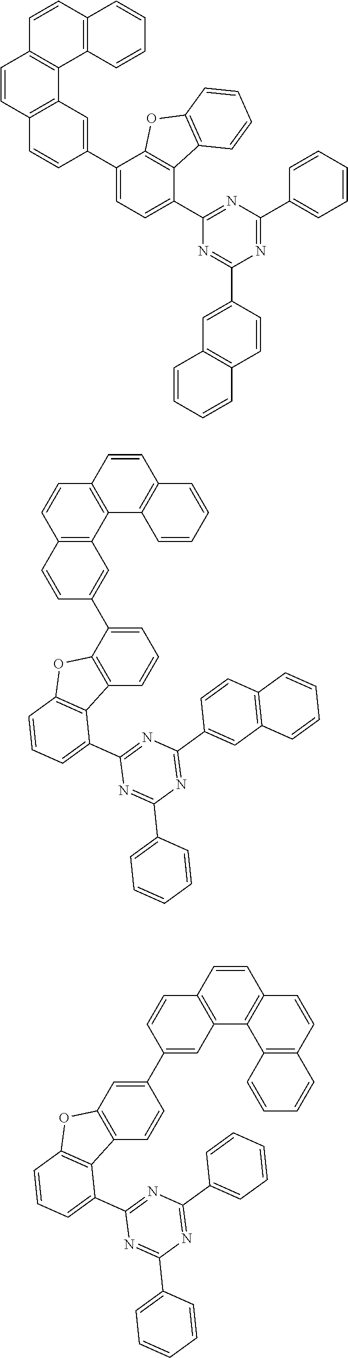 [SMILES: c1ccc(-c2nc(-c3ccc4ccccc4c3)nc(-c3ccc(-c4ccc5ccc6ccc7ccccc7c6c5c4)c4oc5ccccc5c34)n2)cc1 ; c1ccc(-c2nc(-c3ccc4ccccc4c3)nc(-c3cccc4oc5c(-c6ccc7ccc8ccc9ccccc9c8c7c6)cccc5c34)n2)cc1 ; c1ccc(-c2nc(-c3ccccc3)nc(-c3cccc4oc5cc(-c6ccc7ccc8ccc9ccccc9c8c7c6)ccc5c34)n2)cc1]

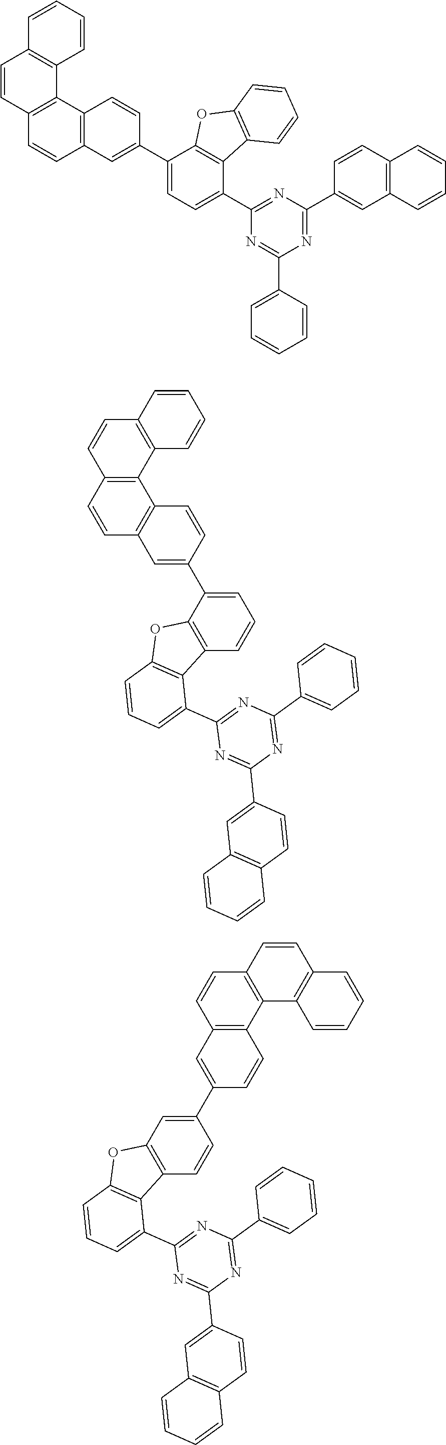

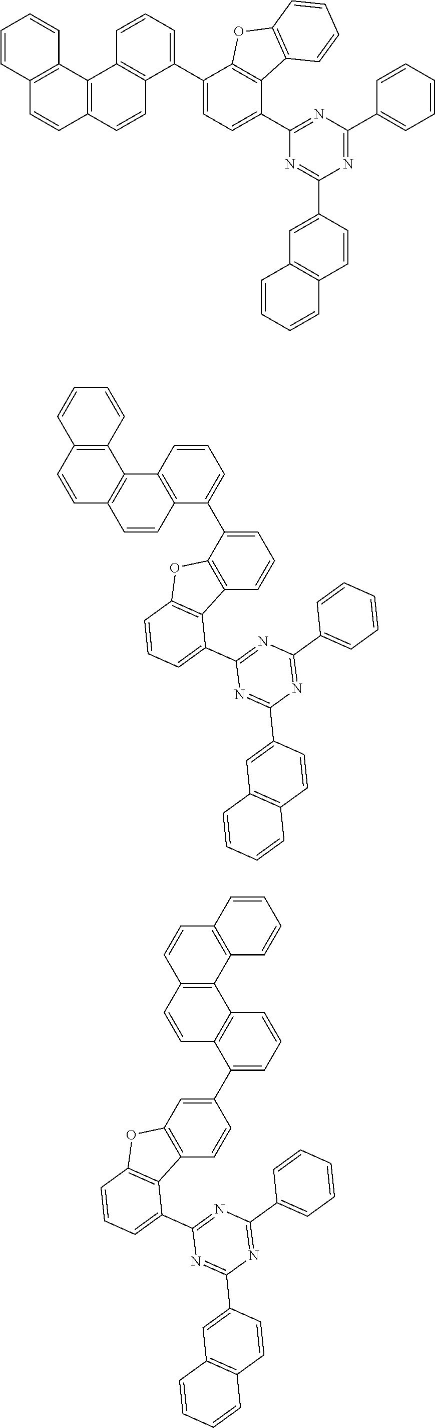

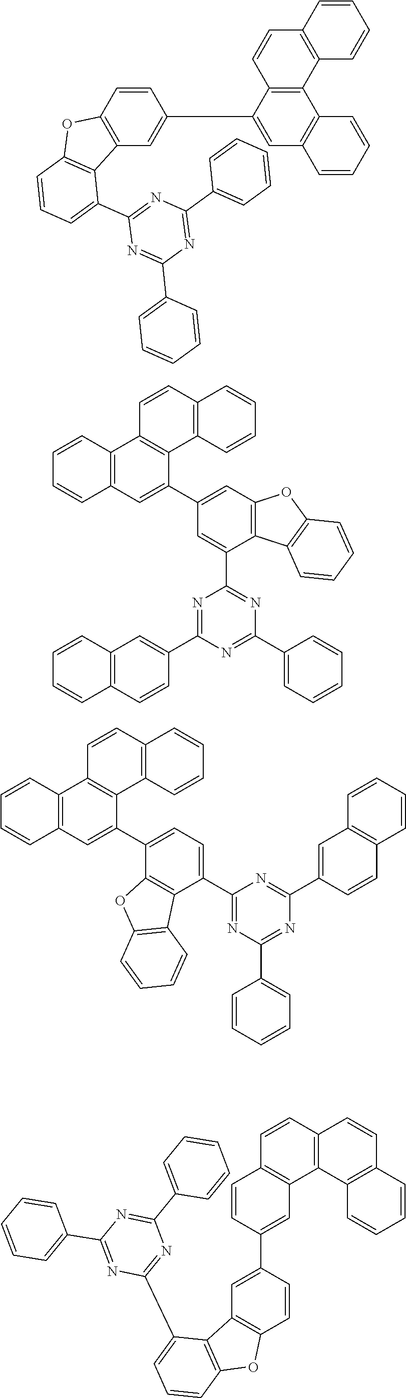

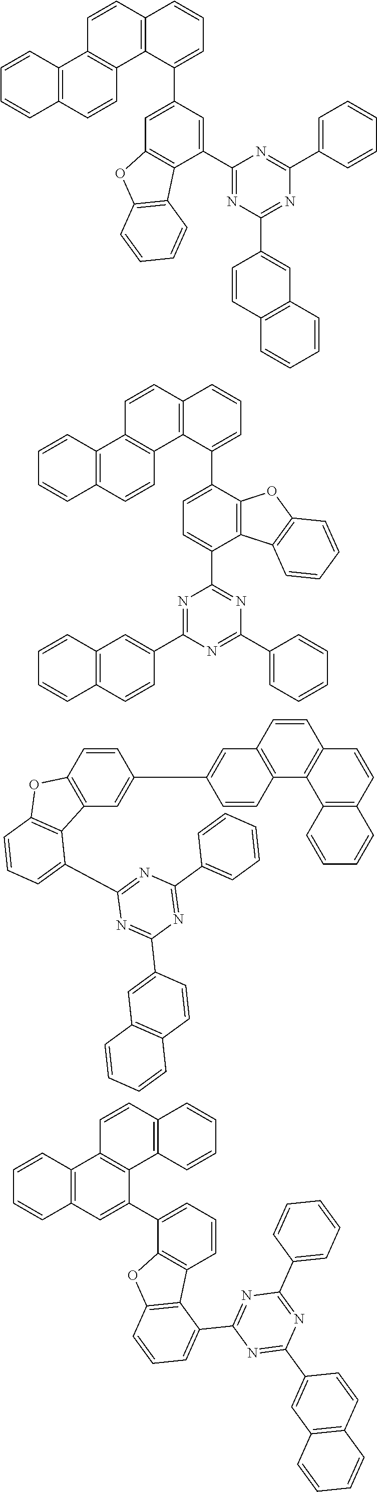

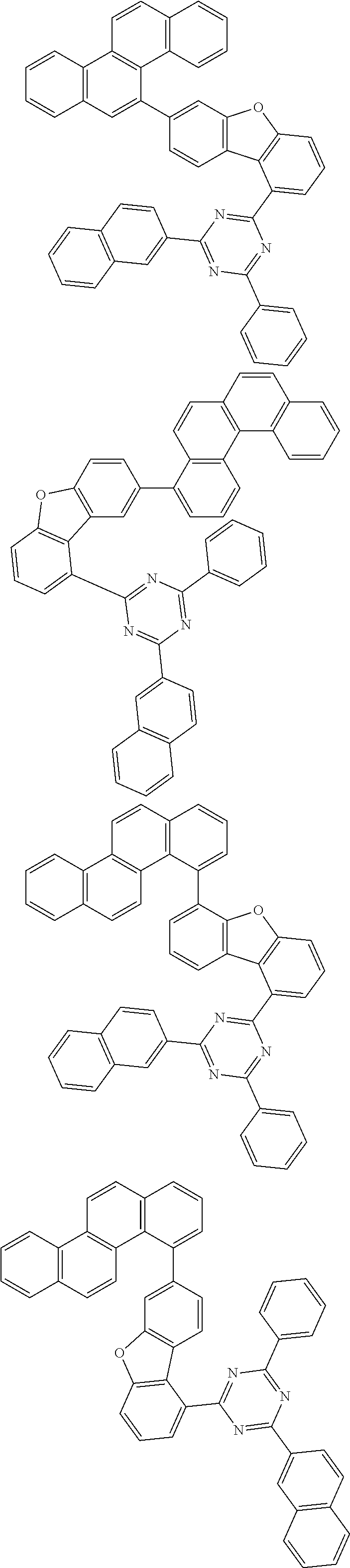

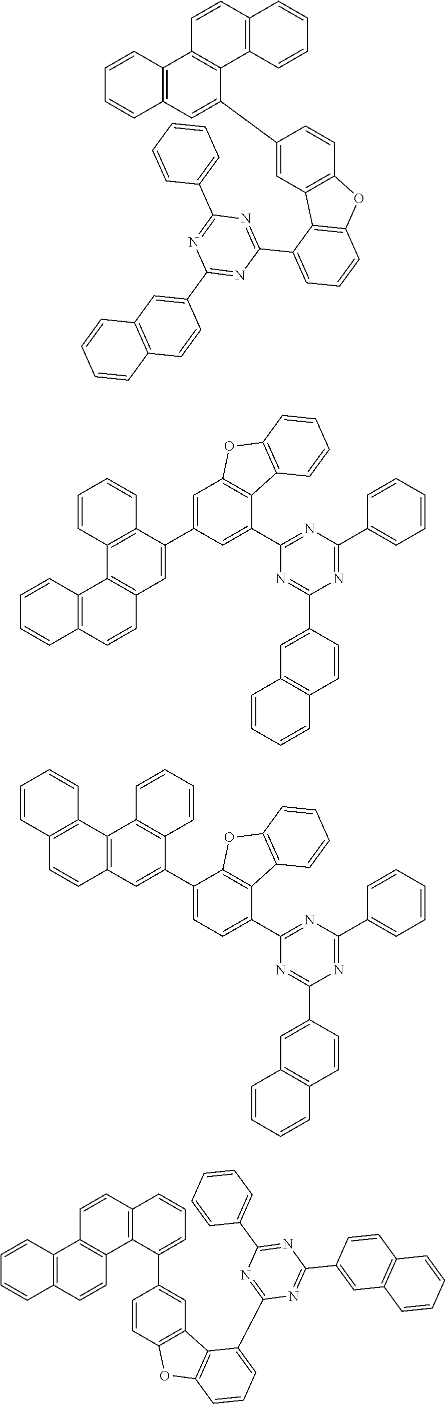 [SMILES: c1ccc(-c2nc(-c3ccc4ccccc4c3)nc(-c3cccc4oc5ccc(-c6cc7ccccc7c7ccc8ccccc8c67)cc5c34)n2)cc1 ; c1ccc(-c2nc(-c3ccc4ccccc4c3)nc(-c3cc(-c4cc5ccc6ccccc6c5c5ccccc45)cc4oc5ccccc5c34)n2)cc1 ; c1ccc(-c2nc(-c3ccc4ccccc4c3)nc(-c3ccc(-c4cc5ccc6ccccc6c5c5ccccc45)c4oc5ccccc5c34)n2)cc1 ; c1ccc(-c2nc(-c3ccc4ccccc4c3)nc(-c3cccc4oc5ccc(-c6cccc7ccc8c9ccccc9ccc8c67)cc5c34)n2)cc1]

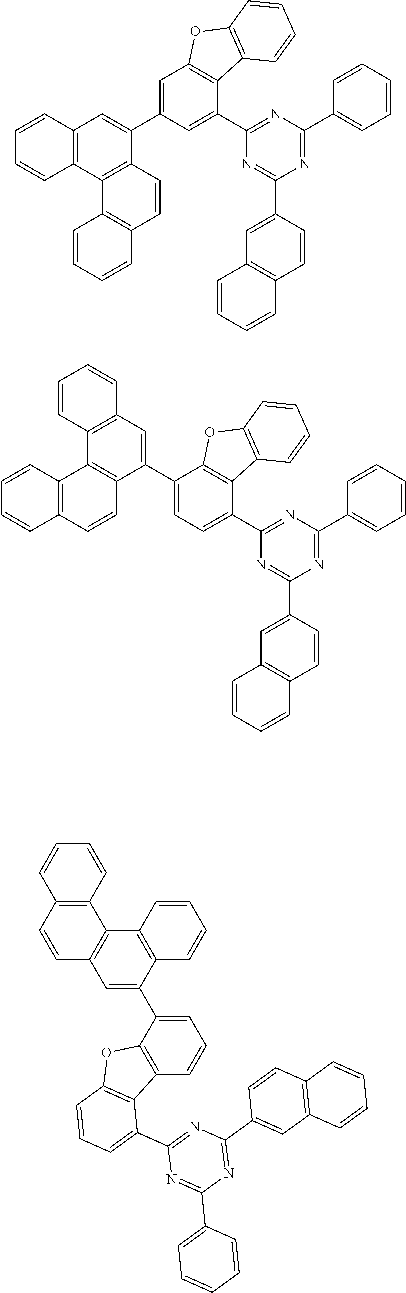 [SMILES: c1ccc(-c2nc(-c3ccc4ccccc4c3)nc(-c3cc(-c4cc5ccccc5c5c4ccc4ccccc45)cc4oc5ccccc5c34)n2)cc1 ; c1ccc(-c2nc(-c3ccc4ccccc4c3)nc(-c3ccc(-c4cc5ccccc5c5c4ccc4ccccc45)c4oc5ccccc5c34)n2)cc1 ; c1ccc(-c2nc(-c3ccc4ccccc4c3)nc(-c3cccc4oc5c(-c6cc7ccc8ccccc8c7c7ccccc67)cccc5c34)n2)cc1]

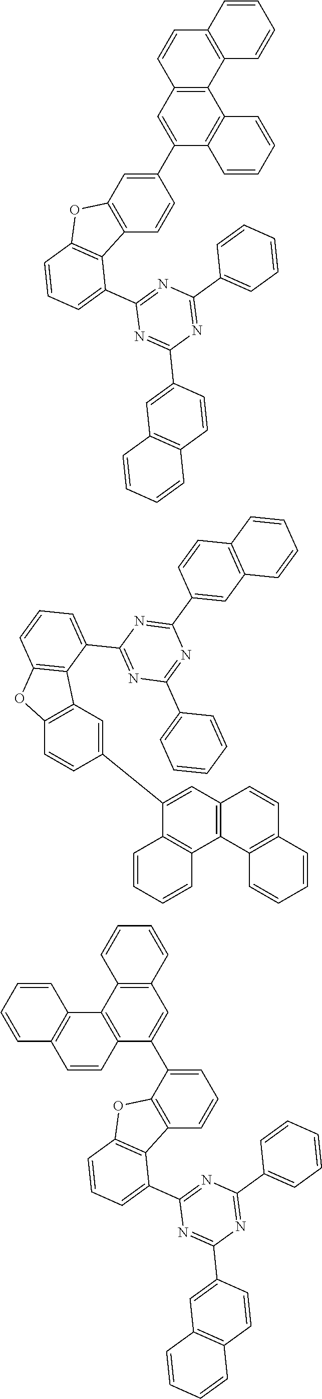

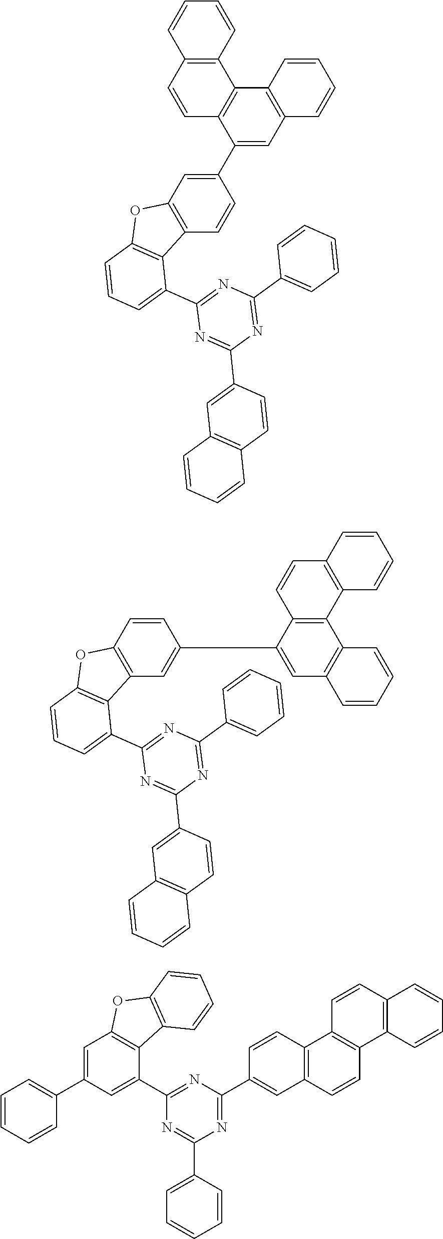

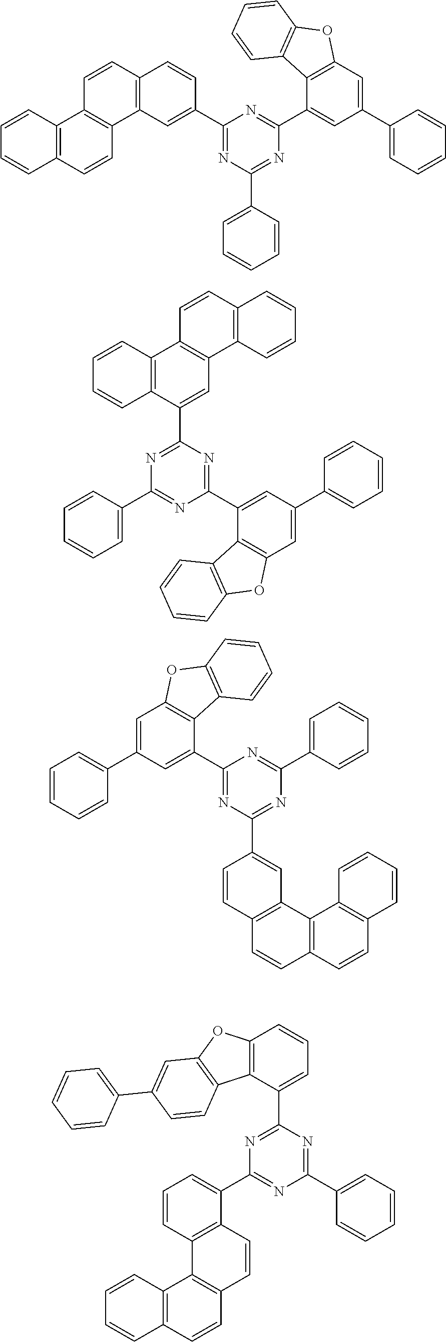

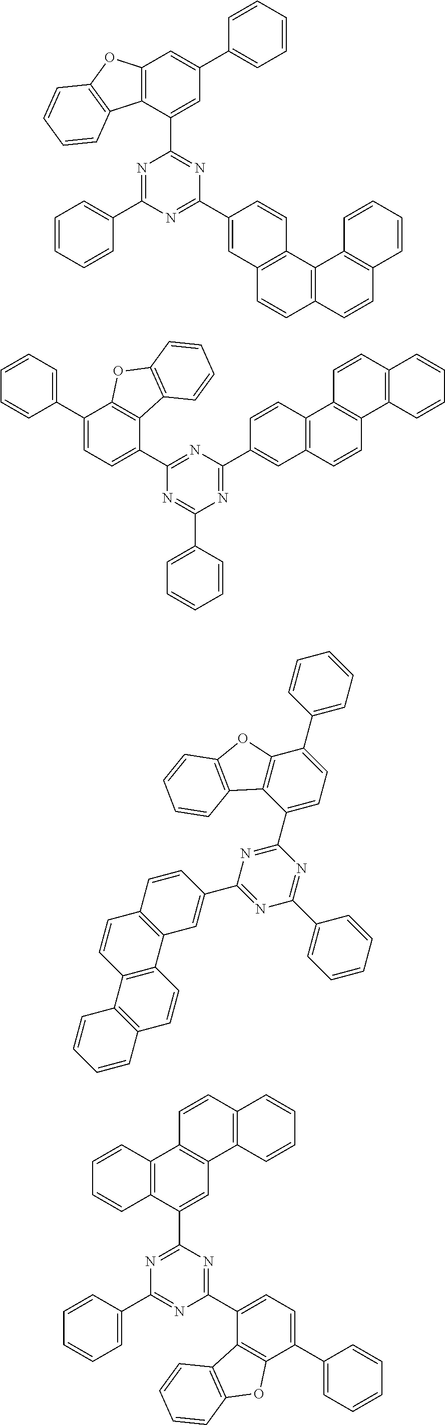

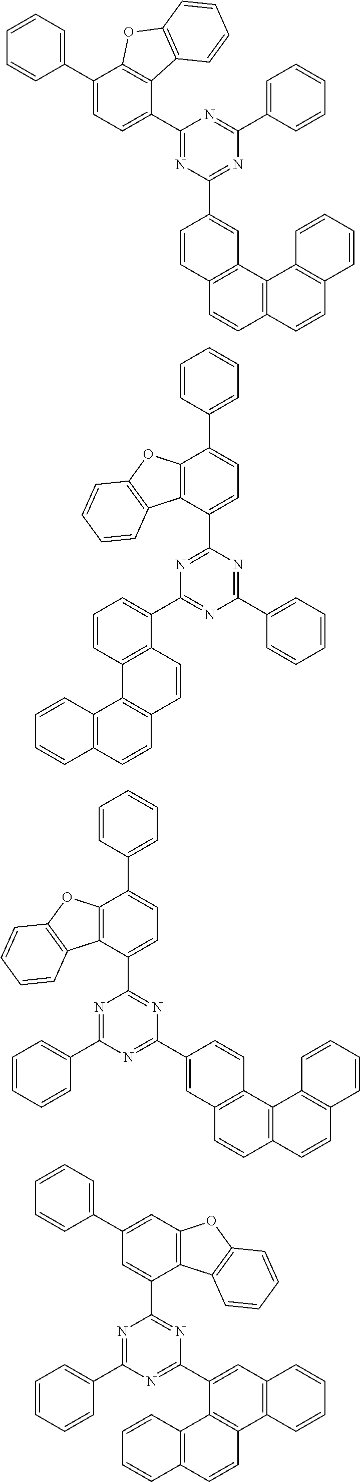

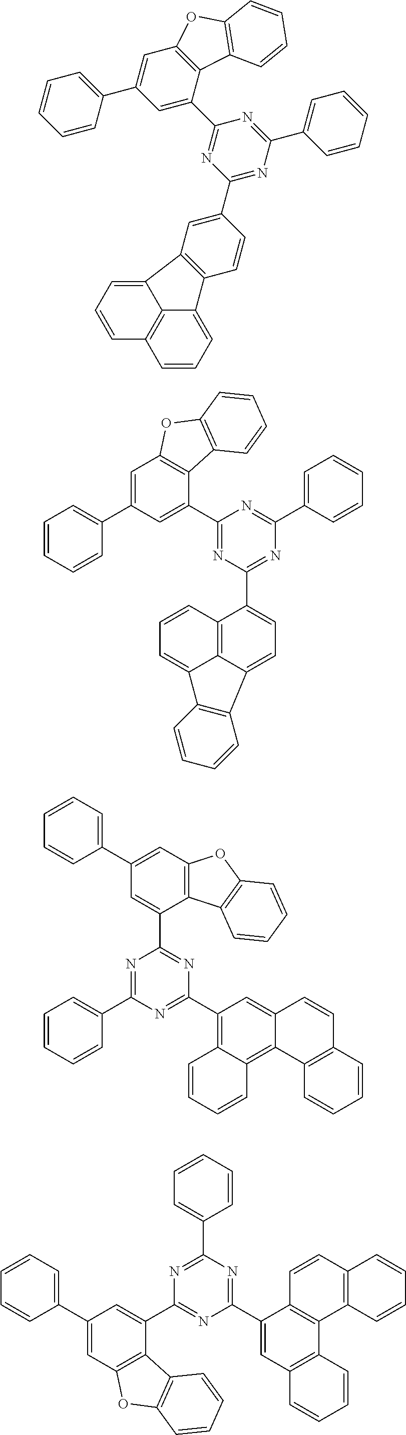

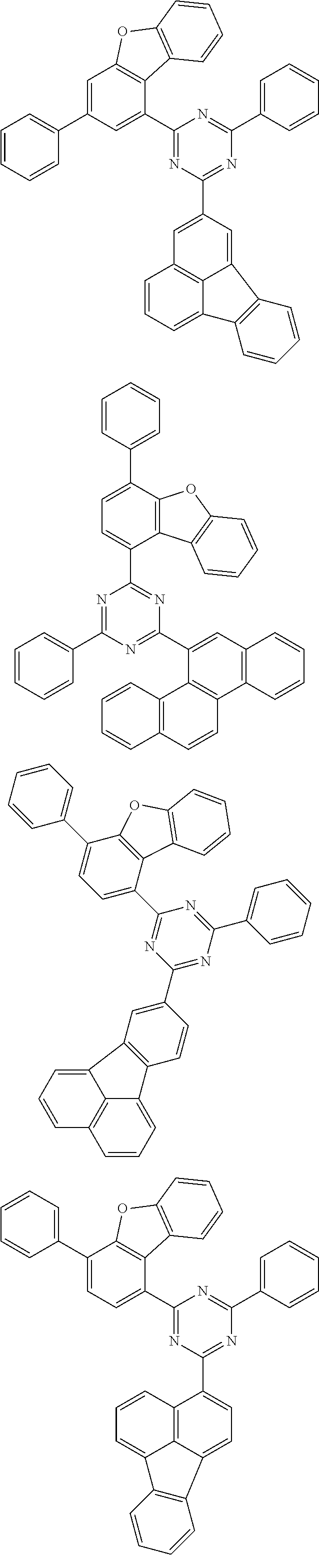

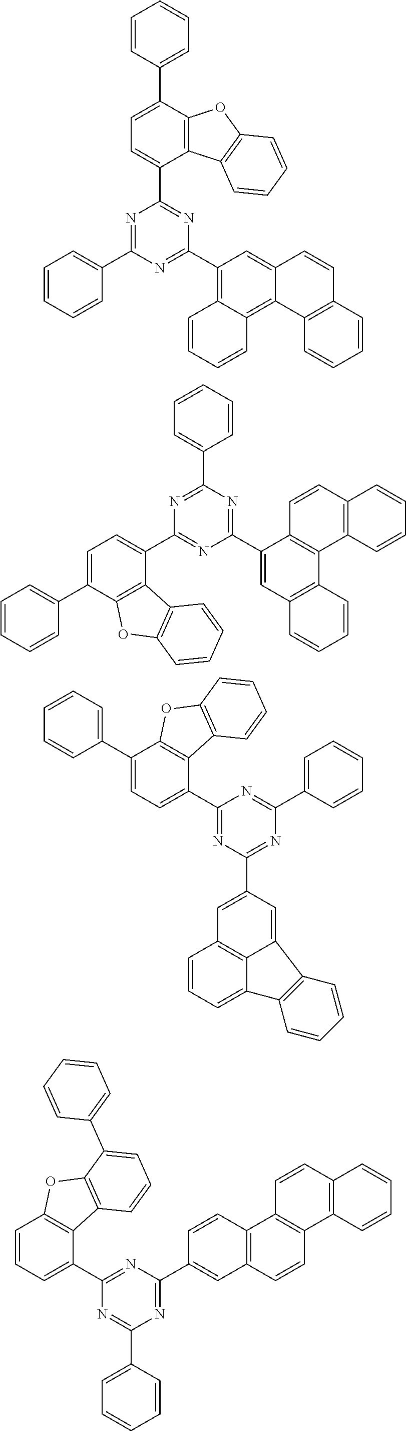

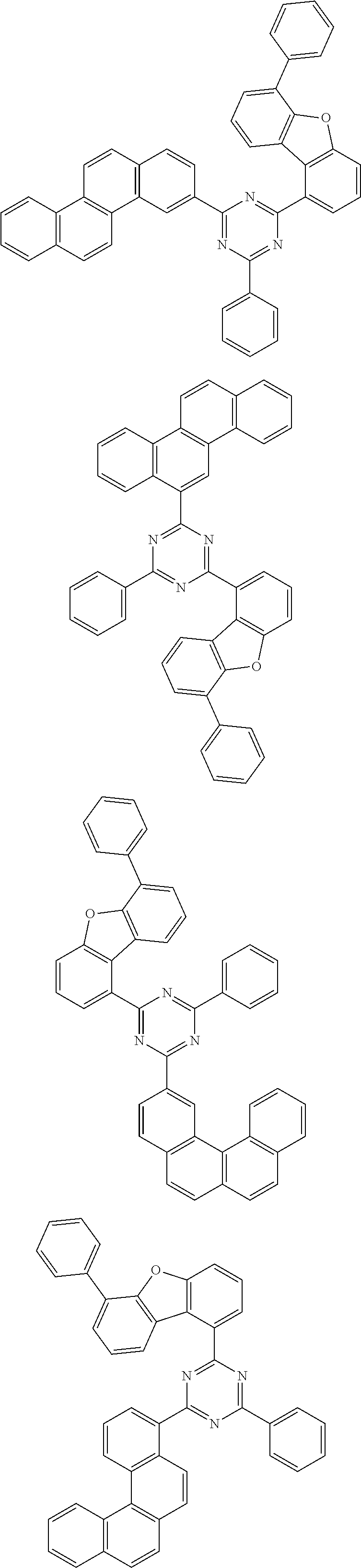

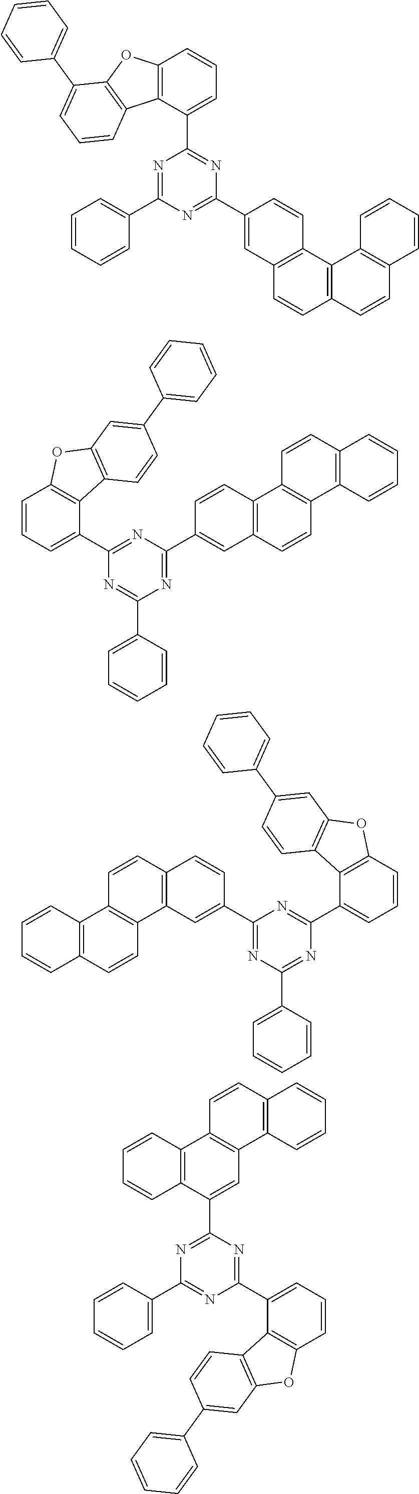

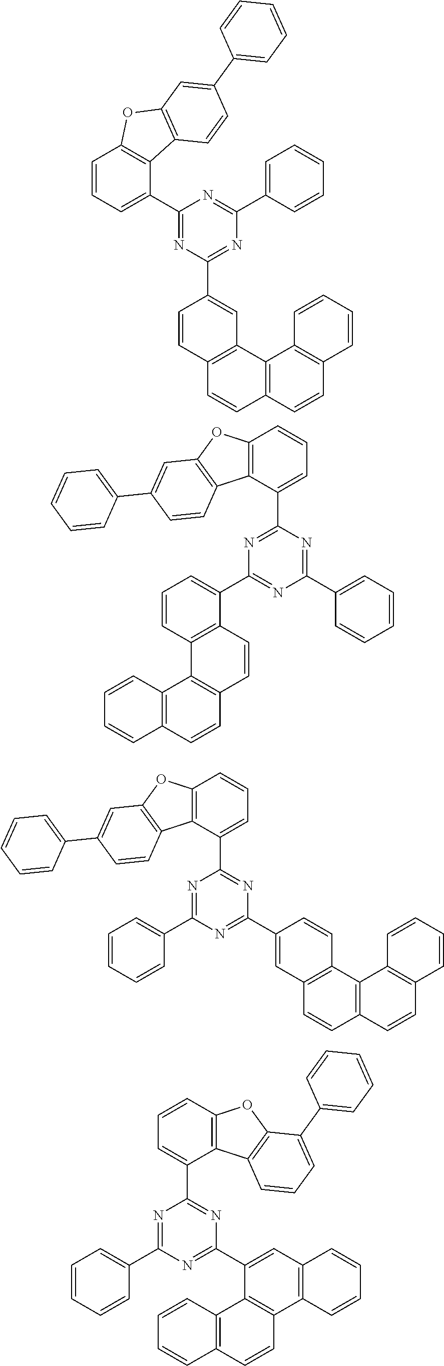

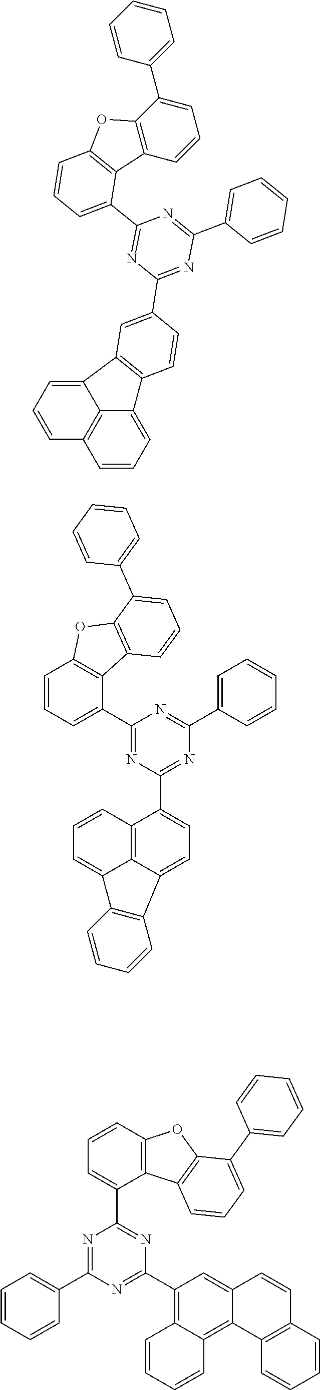

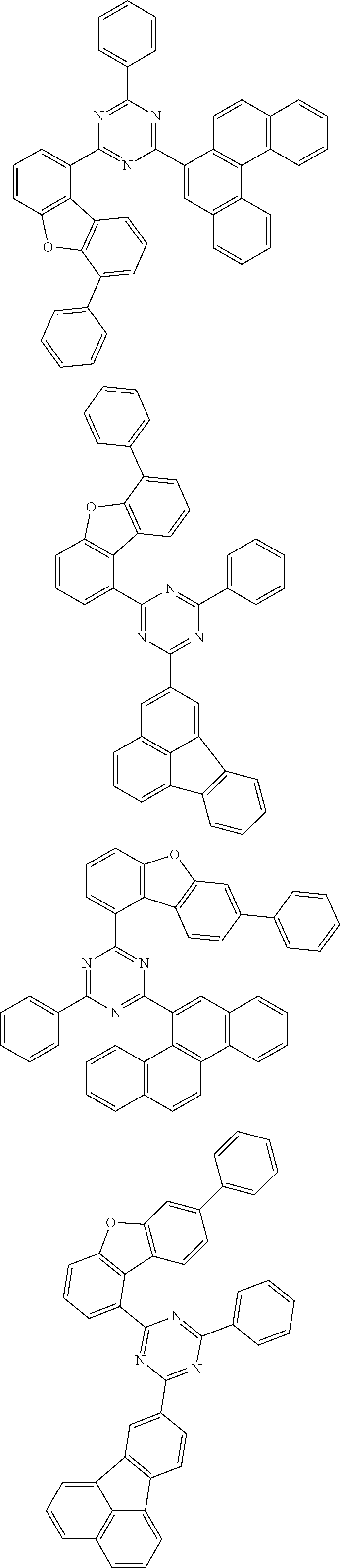

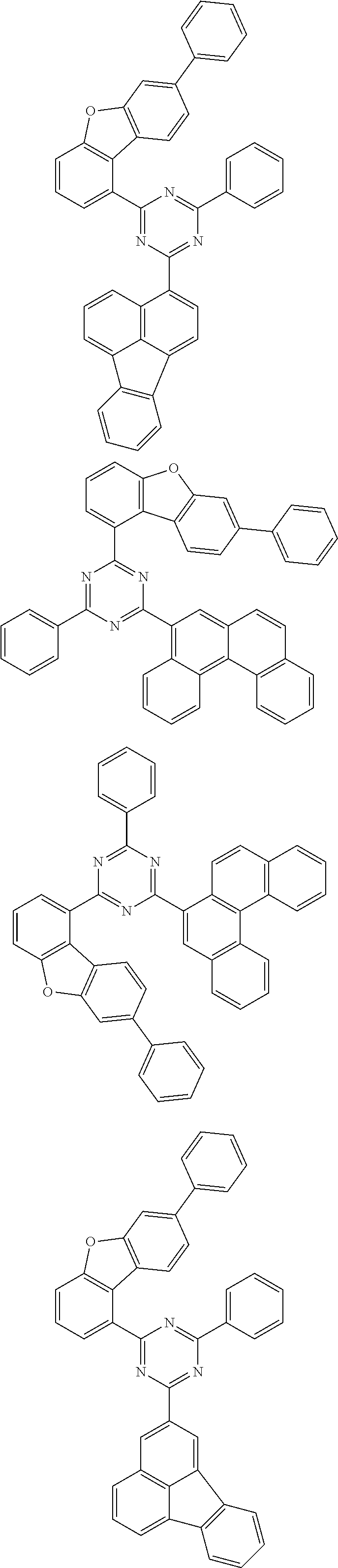

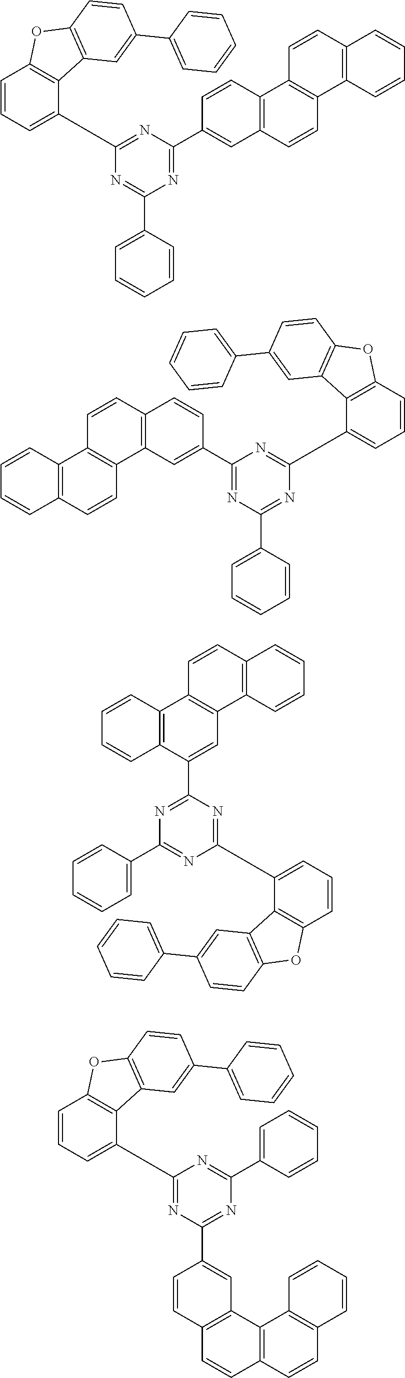

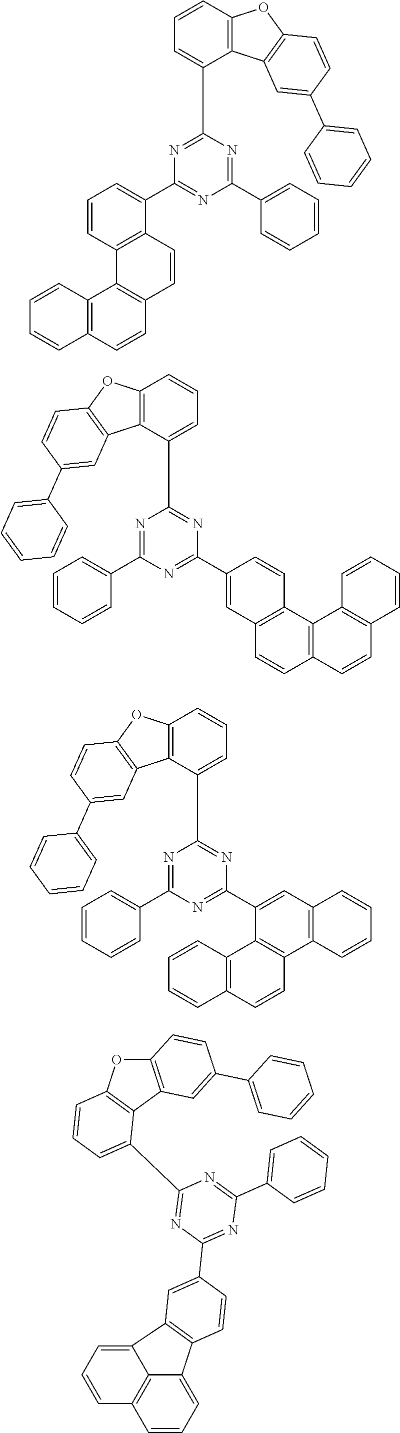

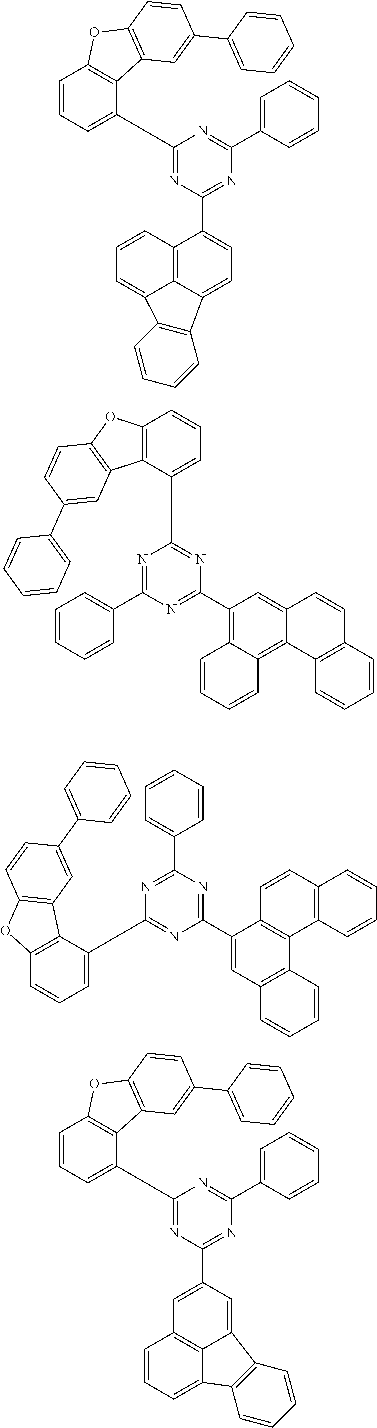

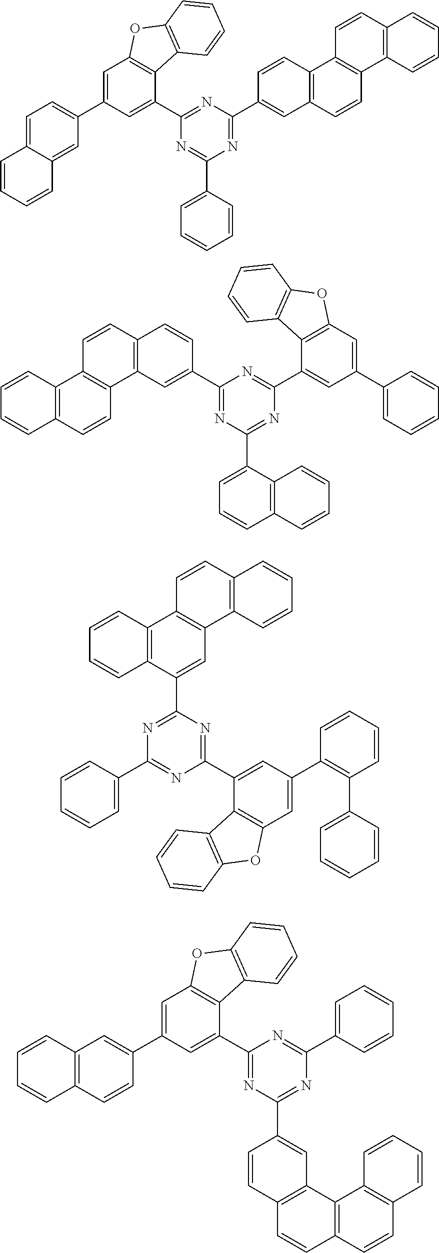

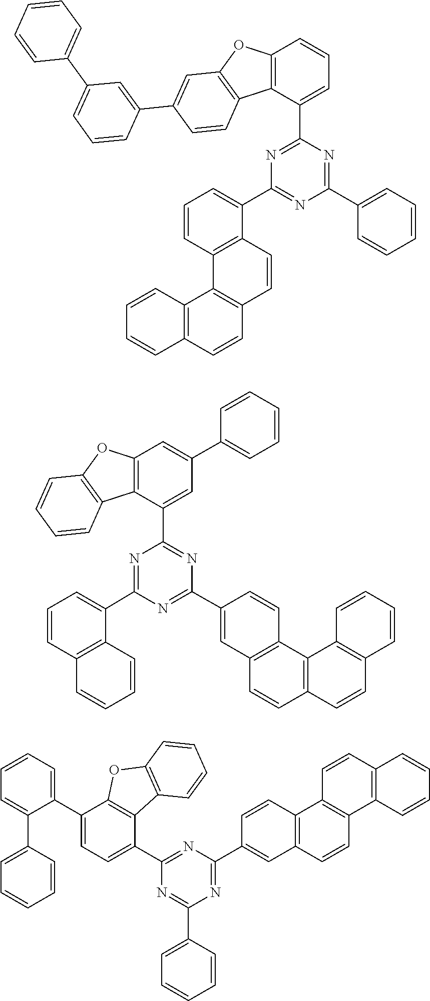 [SMILES: c1ccc(-c2cccc(-c3ccc4c(c3)oc3cccc(-c5nc(-c6ccccc6)nc(-c6cccc7c6ccc6ccc8ccccc8c67)n5)c34)c2)cc1 ; c1ccc(-c2cc(-c3nc(-c4ccc5c(ccc6ccc7ccccc7c65)c4)nc(-c4cccc5ccccc45)n3)c3c(c2)oc2ccccc23)cc1 ; c1ccc(-c2nc(-c3ccc4c(ccc5c6ccccc6ccc45)c3)nc(-c3ccc(-c4ccccc4-c4ccccc4)c4oc5ccccc5c34)n2)cc1]

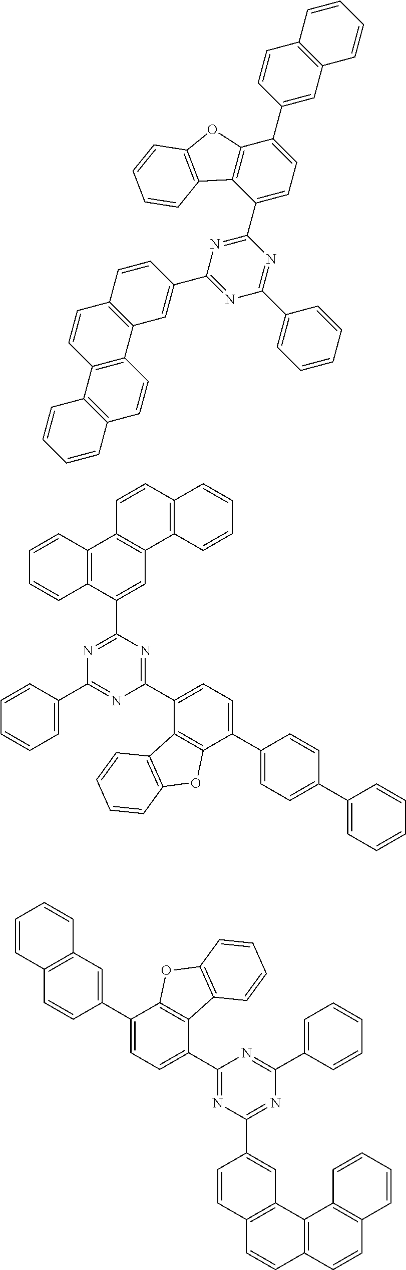 [SMILES: c1ccc(-c2nc(-c3ccc4ccc5c6ccccc6ccc5c4c3)nc(-c3ccc(-c4ccc5ccccc5c4)c4oc5ccccc5c34)n2)cc1 ; c1ccc(-c2ccc(-c3ccc(-c4nc(-c5ccccc5)nc(-c5cc6c7ccccc7ccc6c6ccccc56)n4)c4c3oc3ccccc34)cc2)cc1 ; c1ccc(-c2nc(-c3ccc4ccc5ccc6ccccc6c5c4c3)nc(-c3ccc(-c4ccc5ccccc5c4)c4oc5ccccc5c34)n2)cc1]

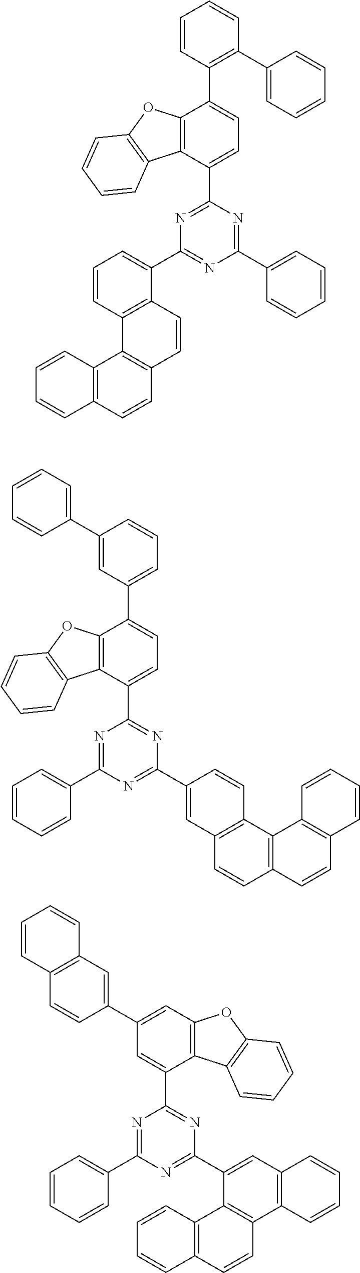 [SMILES: c1ccc(-c2nc(-c3cccc4c3ccc3ccc5ccccc5c34)nc(-c3ccc(-c4ccccc4-c4ccccc4)c4oc5ccccc5c34)n2)cc1 ; c1ccc(-c2cccc(-c3ccc(-c4nc(-c5ccccc5)nc(-c5ccc6c(ccc7ccc8ccccc8c76)c5)n4)c4c3oc3ccccc34)c2)cc1 ; c1ccc(-c2nc(-c3cc(-c4ccc5ccccc5c4)cc4oc5ccccc5c34)nc(-c3cc4ccccc4c4ccc5ccccc5c34)n2)cc1]

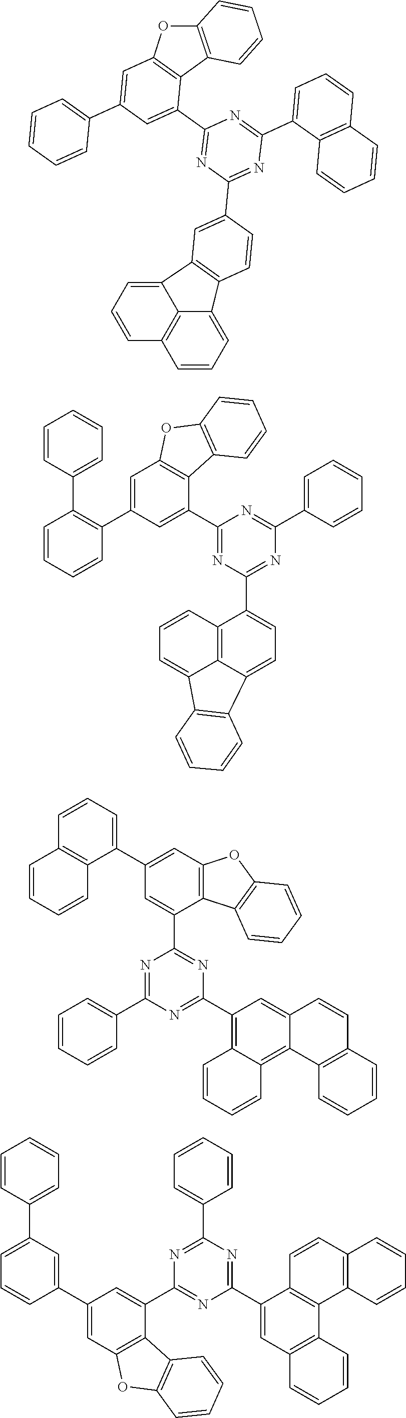

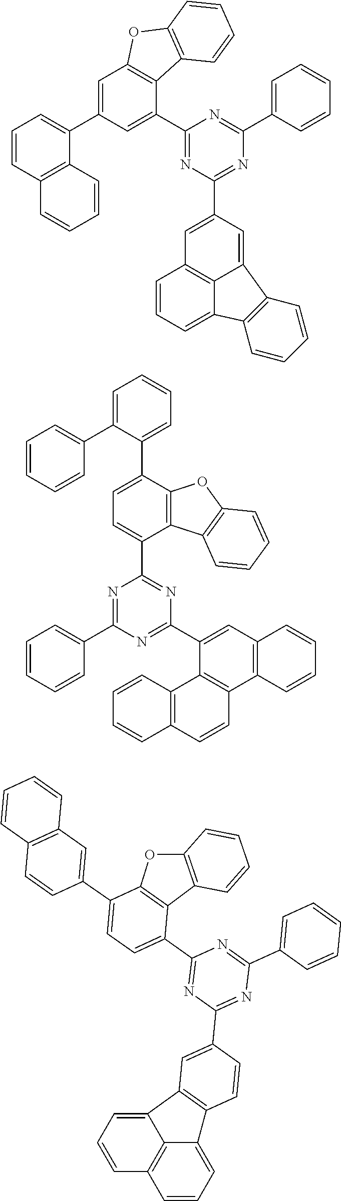 [SMILES: c1ccc(-c2nc(-c3cc4c5c(cccc5c3)-c3ccccc3-4)nc(-c3cc(-c4cccc5ccccc45)cc4oc5ccccc5c34)n2)cc1 ; c1ccc(-c2nc(-c3ccc(-c4ccccc4-c4ccccc4)c4oc5ccccc5c34)nc(-c3cc4ccccc4c4ccc5ccccc5c34)n2)cc1 ; c1ccc(-c2nc(-c3ccc4c(c3)-c3cccc5cccc-4c35)nc(-c3ccc(-c4ccc5ccccc5c4)c4oc5ccccc5c34)n2)cc1]

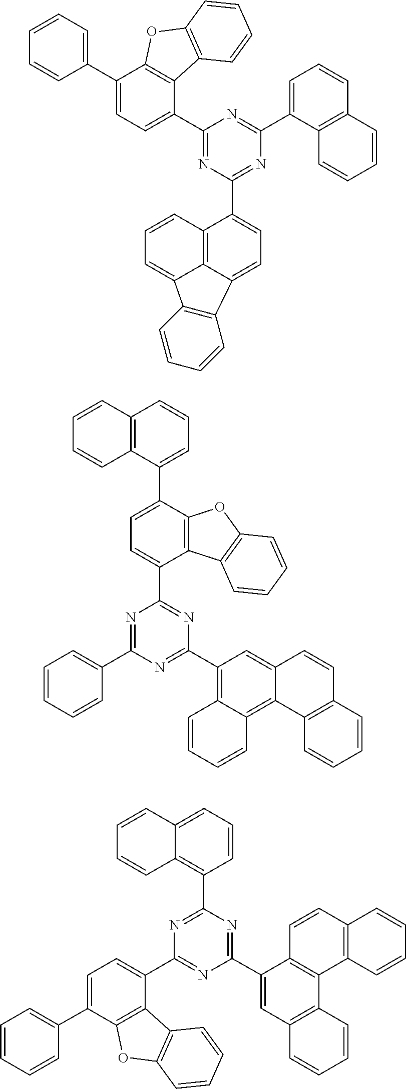

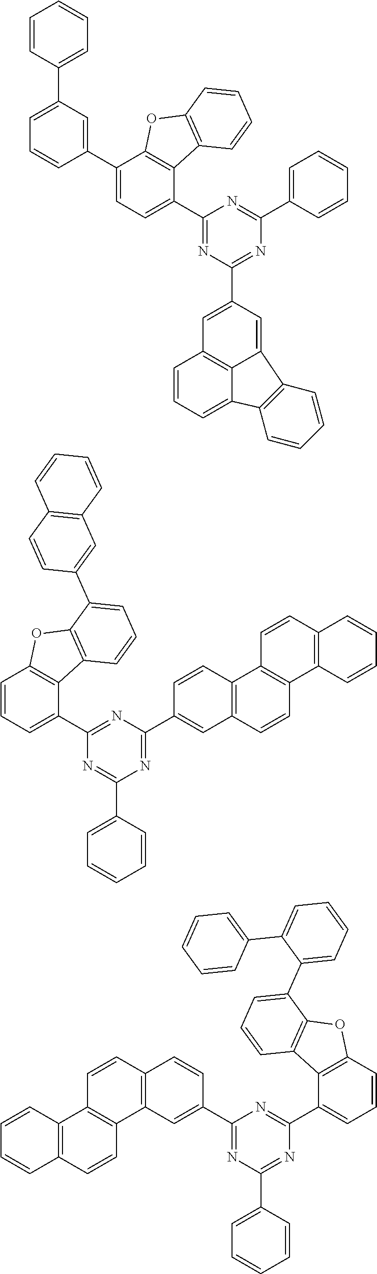 [SMILES: c1ccc(-c2cccc(-c3ccc(-c4nc(-c5ccccc5)nc(-c5cc6c7c(cccc7c5)-c5ccccc5-6)n4)c4c3oc3ccccc34)c2)cc1 ; c1ccc(-c2nc(-c3ccc4c(ccc5c6ccccc6ccc45)c3)nc(-c3cccc4oc5c(-c6ccc7ccccc7c6)cccc5c34)n2)cc1 ; c1ccc(-c2nc(-c3ccc4ccc5c6ccccc6ccc5c4c3)nc(-c3cccc4oc5c(-c6ccccc6-c6ccccc6)cccc5c34)n2)cc1]

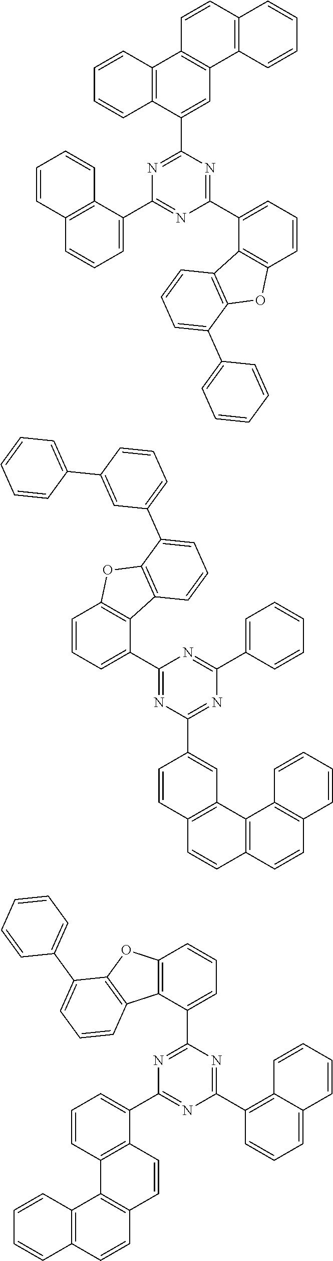 [SMILES: c1ccc(-c2cccc3c2oc2cccc(-c4nc(-c5cccc6ccccc56)nc(-c5cc6c7ccccc7ccc6c6ccccc56)n4)c23)cc1 ; c1ccc(-c2cccc(-c3cccc4c3oc3cccc(-c5nc(-c6ccccc6)nc(-c6ccc7ccc8ccc9ccccc9c8c7c6)n5)c34)c2)cc1 ; c1ccc(-c2cccc3c2oc2cccc(-c4nc(-c5cccc6ccccc56)nc(-c5cccc6c5ccc5ccc7ccccc7c56)n4)c23)cc1]

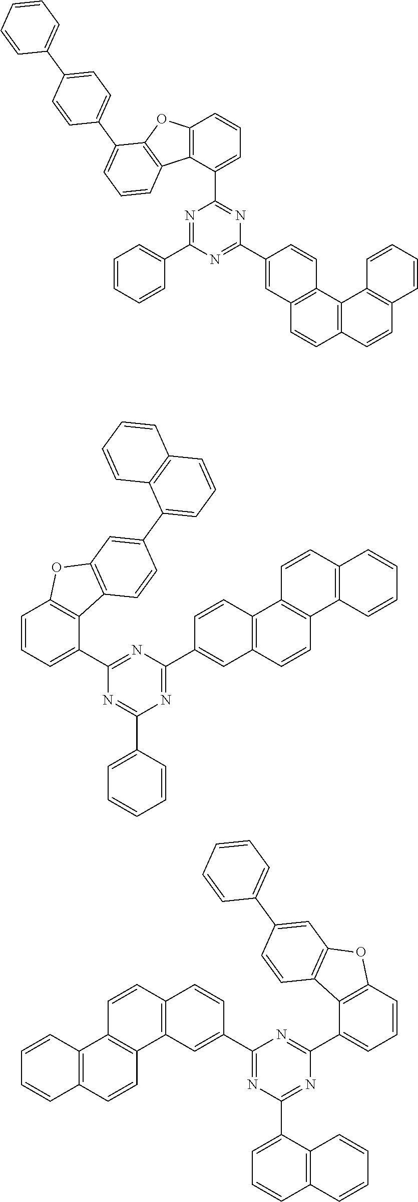 [SMILES: c1ccc(-c2ccc(-c3cccc4c3oc3cccc(-c5nc(-c6ccccc6)nc(-c6ccc7c(ccc8ccc9ccccc9c87)c6)n5)c34)cc2)cc1 ; c1ccc(-c2nc(-c3ccc4c(ccc5c6ccccc6ccc45)c3)nc(-c3cccc4oc5cc(-c6cccc7ccccc67)ccc5c34)n2)cc1 ; c1ccc(-c2ccc3c(c2)oc2cccc(-c4nc(-c5ccc6ccc7c8ccccc8ccc7c6c5)nc(-c5cccc6ccccc56)n4)c23)cc1]

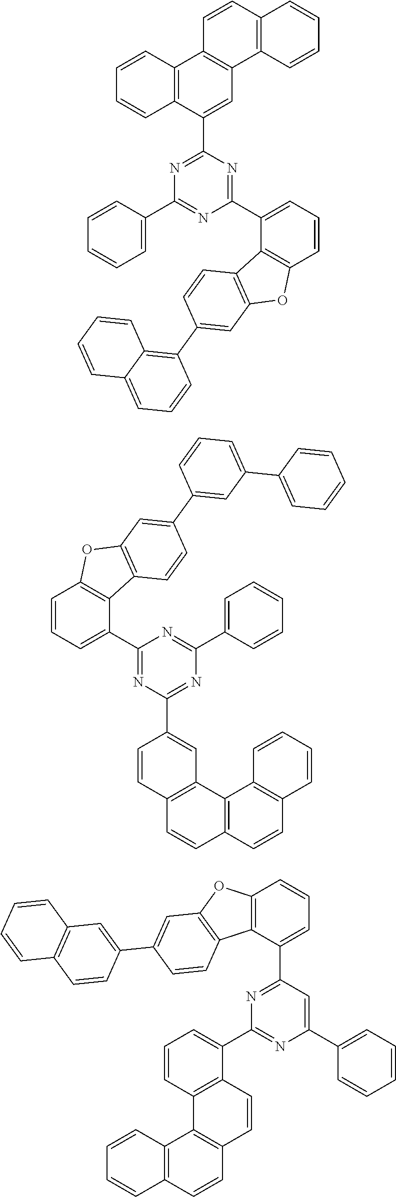

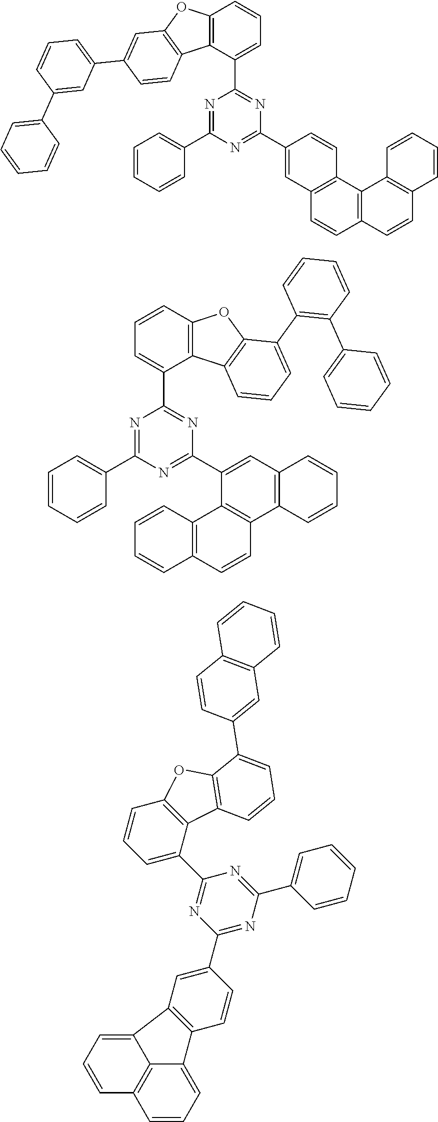

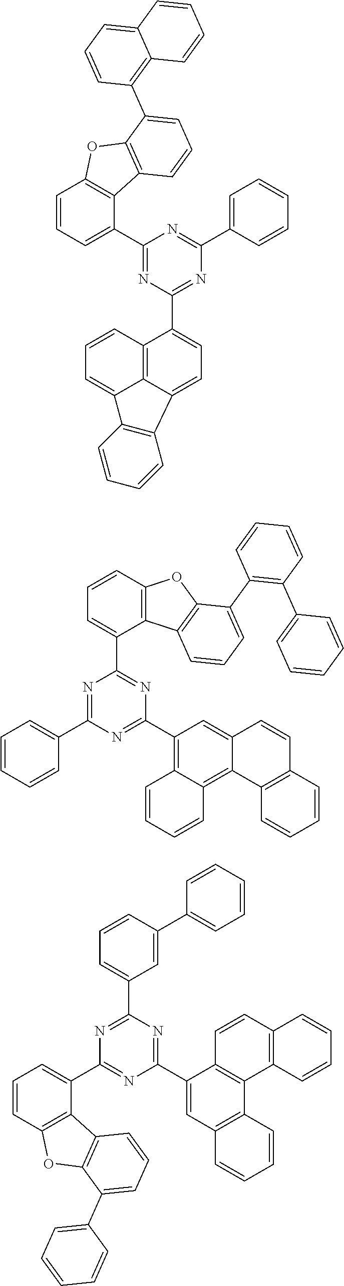

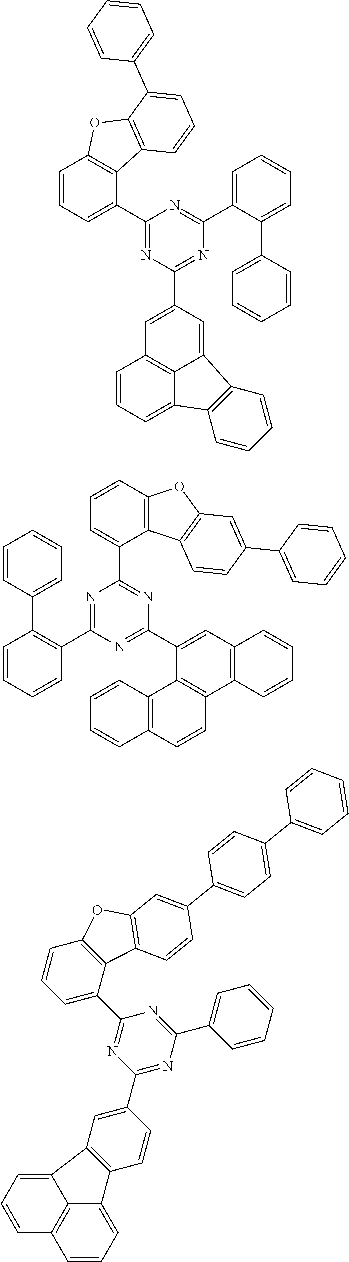 [SMILES: c1ccc(-c2ccccc2-c2nc(-c3cc4c5c(cccc5c3)-c3ccccc3-4)nc(-c3cccc4oc5c(-c6ccccc6)cccc5c34)n2)cc1 ; c1ccc(-c2ccc3c(c2)oc2cccc(-c4nc(-c5ccccc5-c5ccccc5)nc(-c5cc6ccccc6c6ccc7ccccc7c56)n4)c23)cc1 ; c1ccc(-c2ccc(-c3ccc4c(c3)oc3cccc(-c5nc(-c6ccccc6)nc(-c6ccc7c(c6)-c6cccc8cccc-7c68)n5)c34)cc2)cc1]

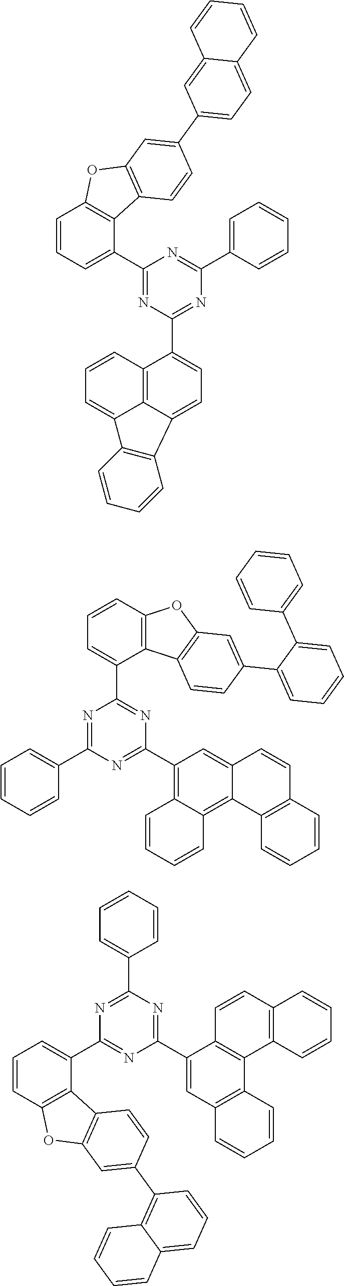

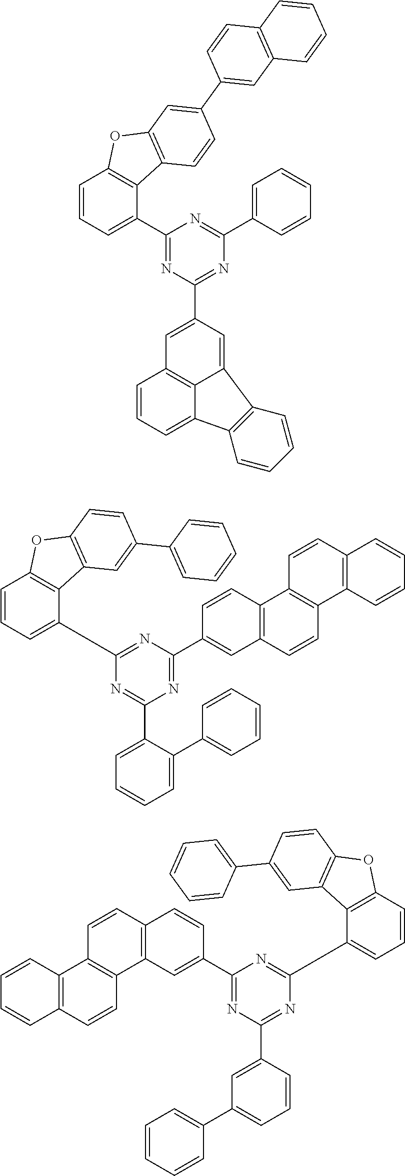 [SMILES: c1ccc(-c2nc(-c3cc4c5c(cccc5c3)-c3ccccc3-4)nc(-c3cccc4oc5cc(-c6ccc7ccccc7c6)ccc5c34)n2)cc1 ; c1ccc(-c2ccc3oc4cccc(-c5nc(-c6ccc7c(ccc8c9ccccc9ccc78)c6)nc(-c6ccccc6-c6ccccc6)n5)c4c3c2)cc1 ; c1ccc(-c2cccc(-c3nc(-c4ccc5ccc6c7ccccc7ccc6c5c4)nc(-c4cccc5oc6ccc(-c7ccccc7)cc6c45)n3)c2)cc1]

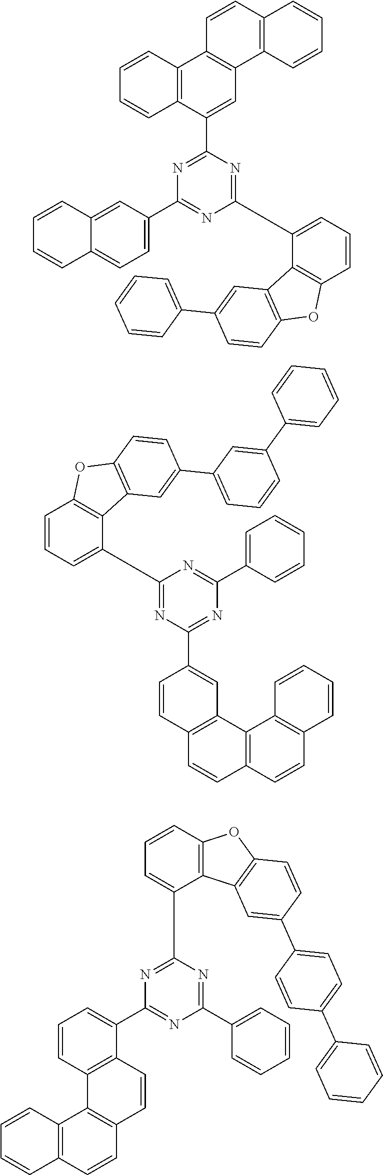

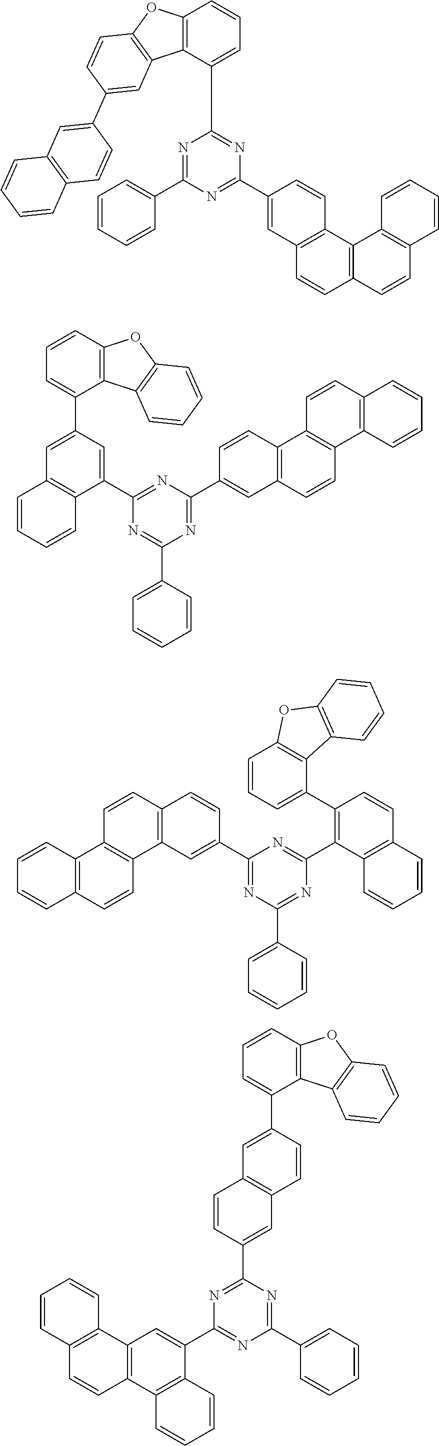

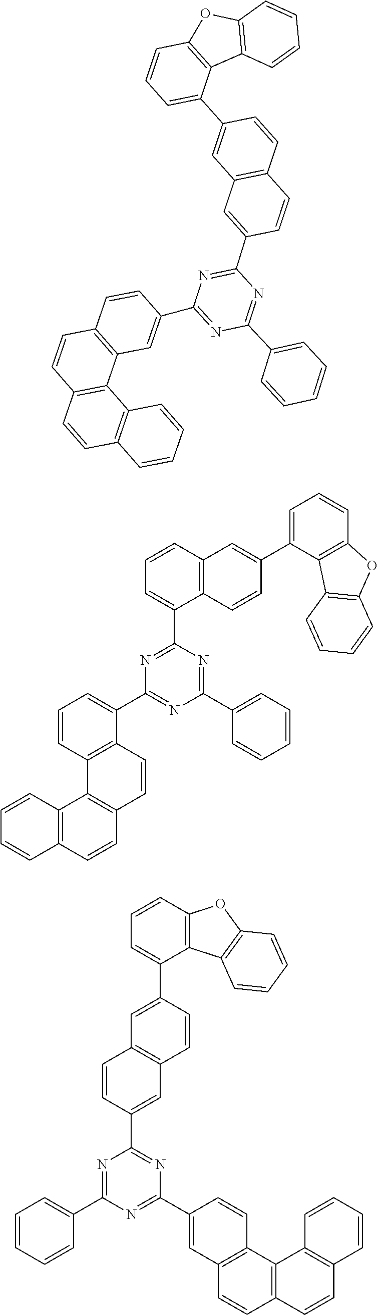 [SMILES: c1ccc(-c2nc(-c3ccc4ccc(-c5cccc6oc7ccccc7c56)cc4c3)nc(-c3ccc4ccc5ccc6ccccc6c5c4c3)n2)cc1 ; c1ccc(-c2nc(-c3cccc4cc(-c5cccc6oc7ccccc7c56)ccc34)nc(-c3cccc4c3ccc3ccc5ccccc5c34)n2)cc1 ; c1ccc(-c2nc(-c3ccc4cc(-c5cccc6oc7ccccc7c56)ccc4c3)nc(-c3ccc4c(ccc5ccc6ccccc6c54)c3)n2)cc1]

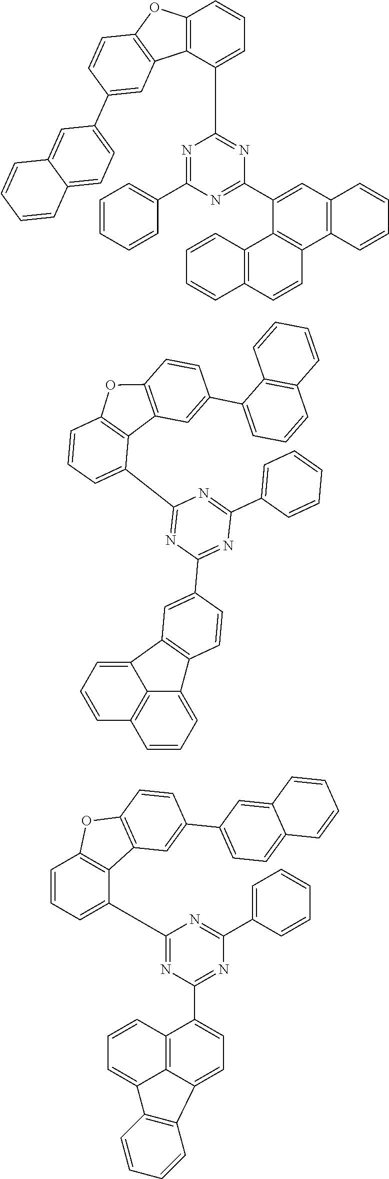

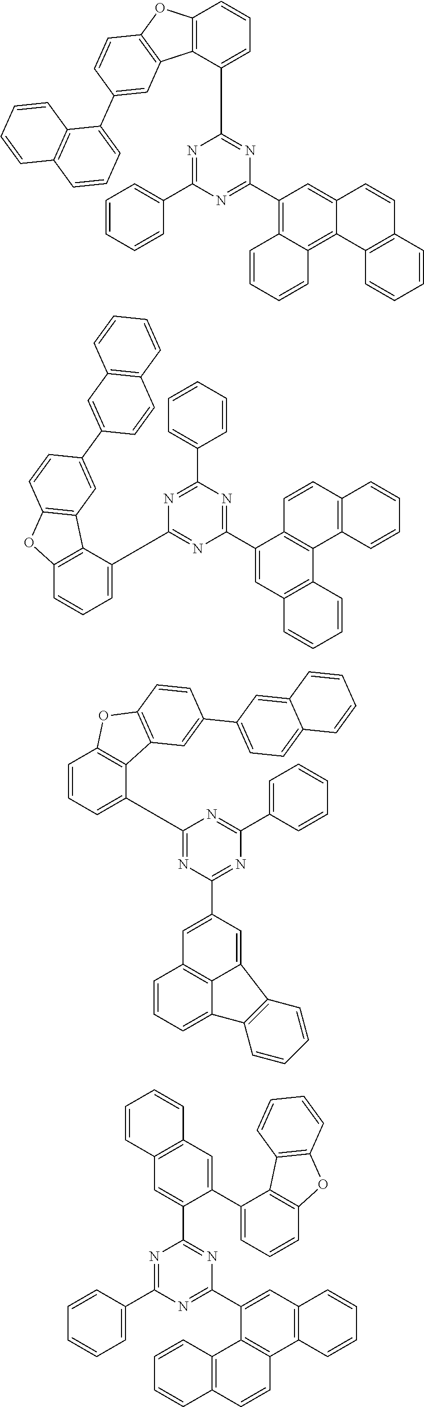

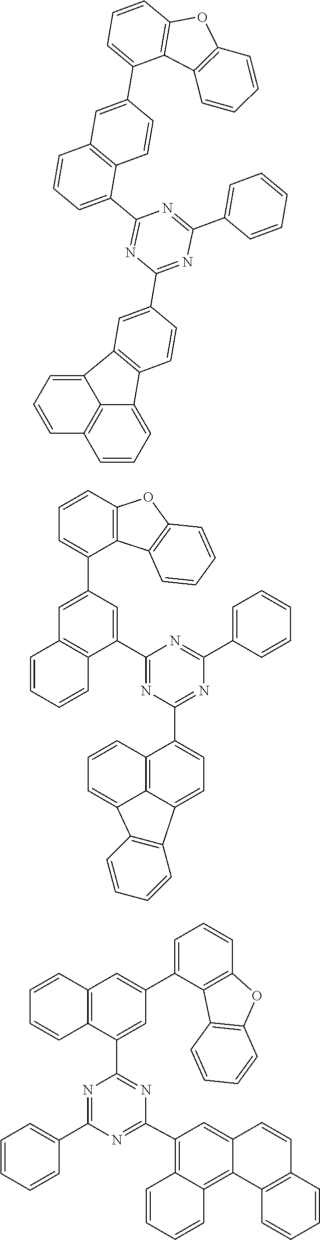

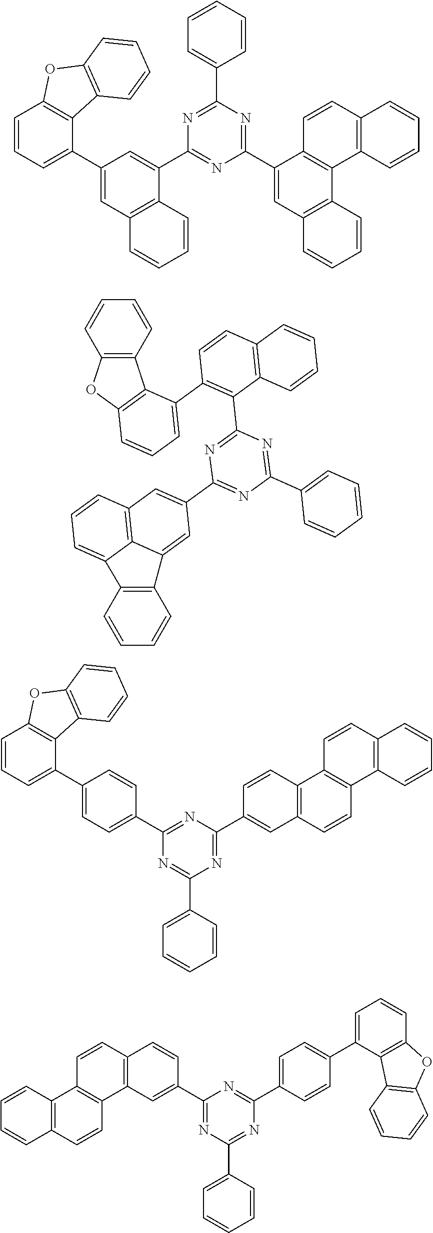

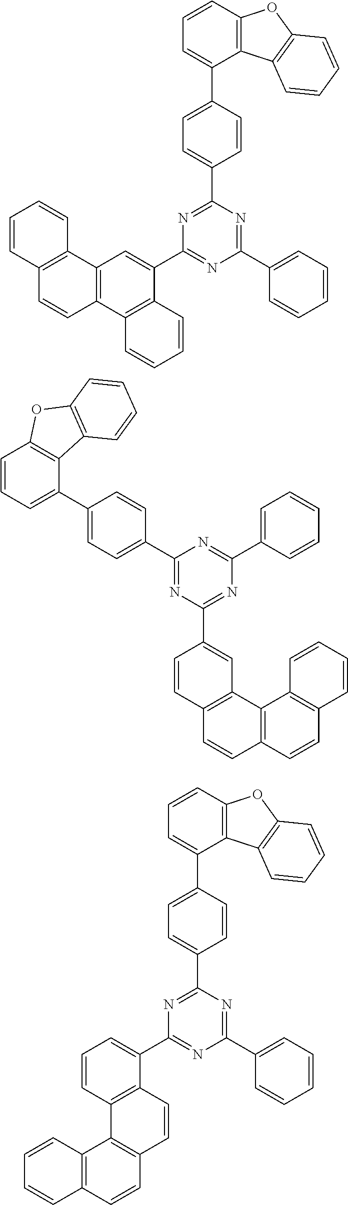

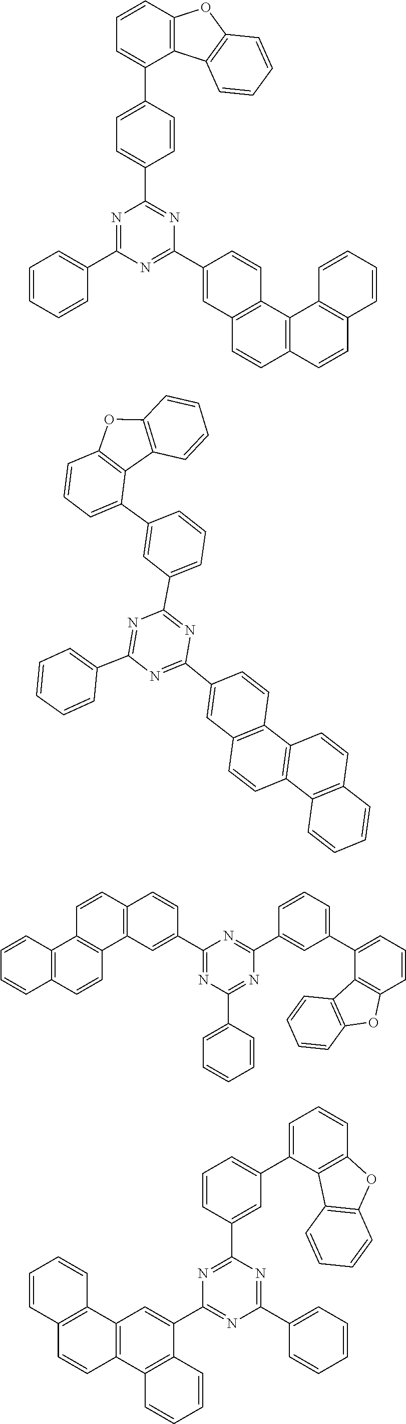 [SMILES: c1ccc(-c2nc(-c3ccc(-c4cccc5oc6ccccc6c45)cc3)nc(-c3ccc4c(ccc5ccc6ccccc6c54)c3)n2)cc1 ; c1ccc(-c2nc(-c3cccc(-c4cccc5oc6ccccc6c45)c3)nc(-c3ccc4c(ccc5c6ccccc6ccc45)c3)n2)cc1 ; c1ccc(-c2nc(-c3cccc(-c4cccc5oc6ccccc6c45)c3)nc(-c3ccc4ccc5c6ccccc6ccc5c4c3)n2)cc1 ; c1ccc(-c2nc(-c3cccc(-c4cccc5oc6ccccc6c45)c3)nc(-c3cc4c5ccccc5ccc4c4ccccc34)n2)cc1]

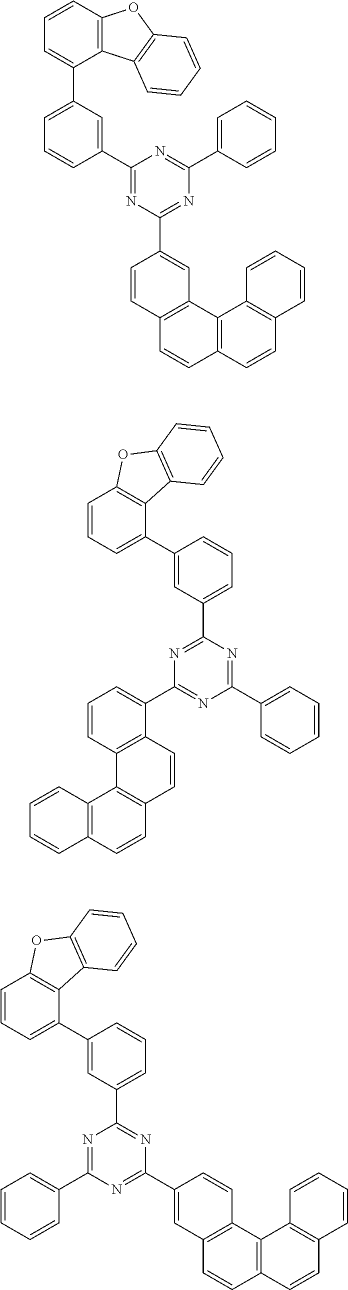 [SMILES: c1ccc(-c2nc(-c3cccc(-c4cccc5oc6ccccc6c45)c3)nc(-c3ccc4ccc5ccc6ccccc6c5c4c3)n2)cc1 ; c1ccc(-c2nc(-c3cccc(-c4cccc5oc6ccccc6c45)c3)nc(-c3cccc4c3ccc3ccc5ccccc5c34)n2)cc1 ; c1ccc(-c2nc(-c3cccc(-c4cccc5oc6ccccc6c45)c3)nc(-c3ccc4c(ccc5ccc6ccccc6c54)c3)n2)cc1]

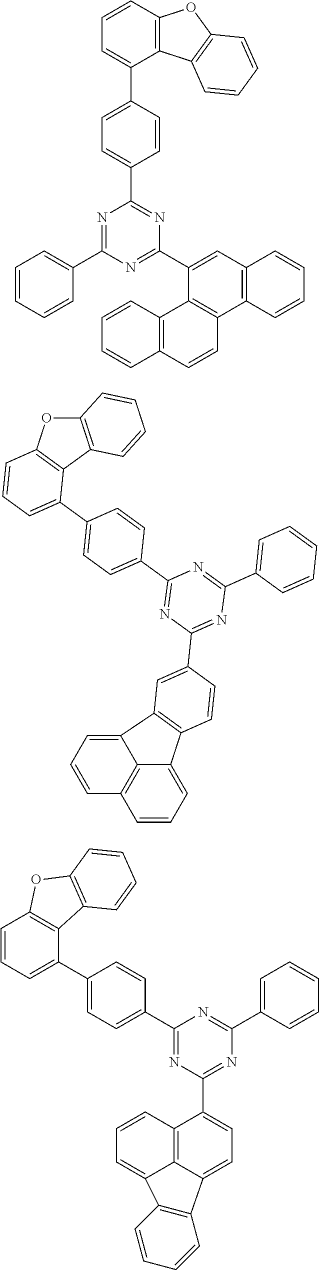

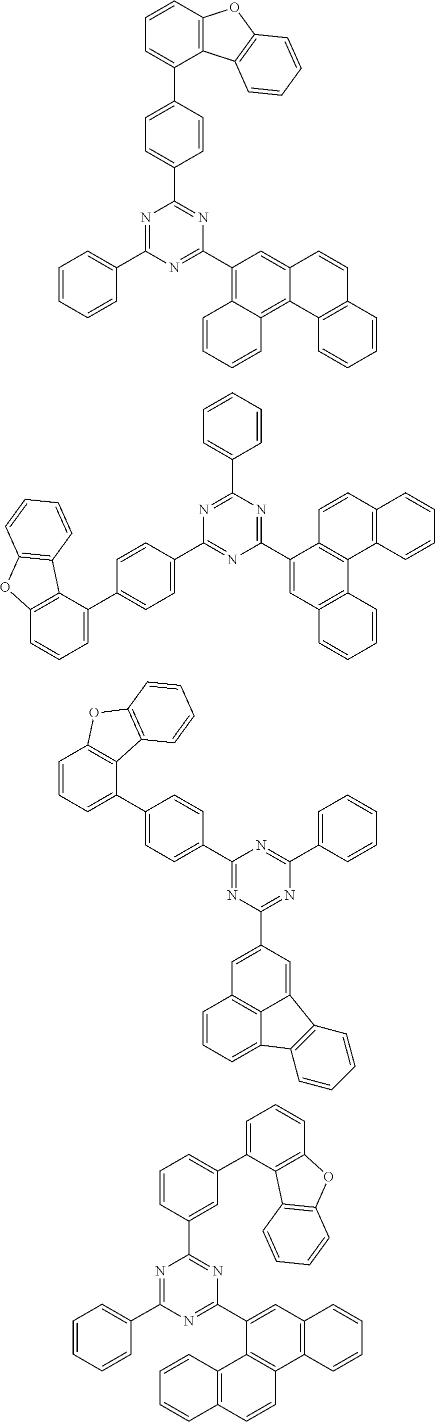

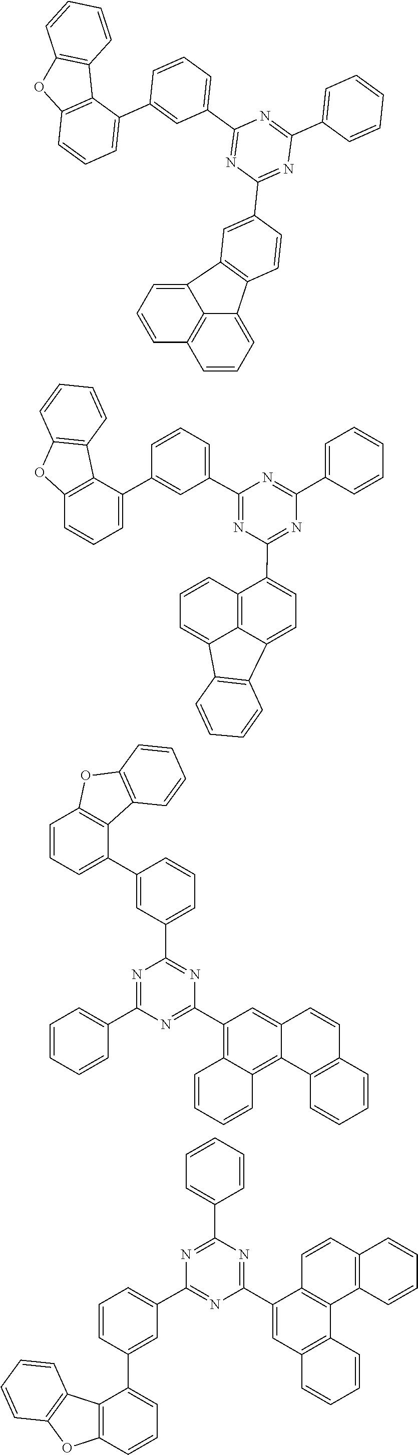

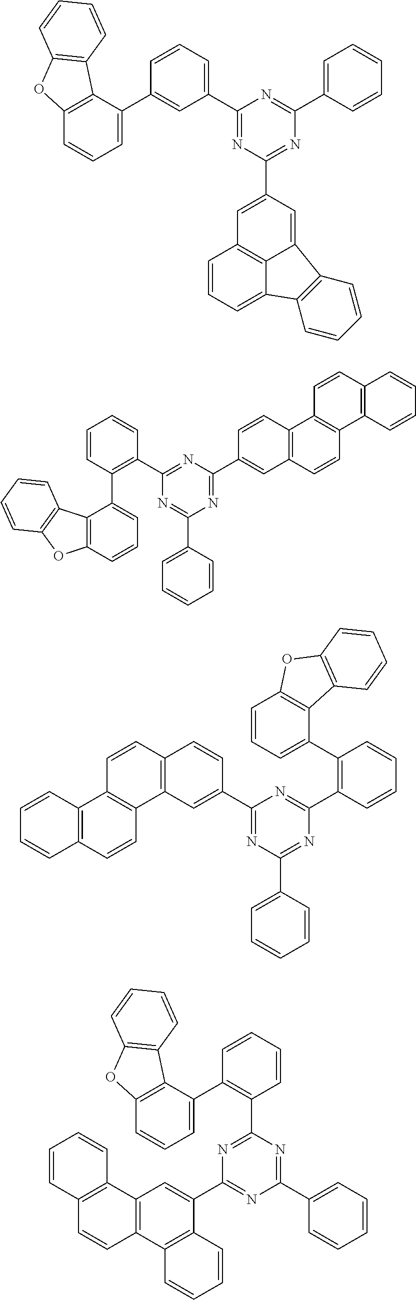

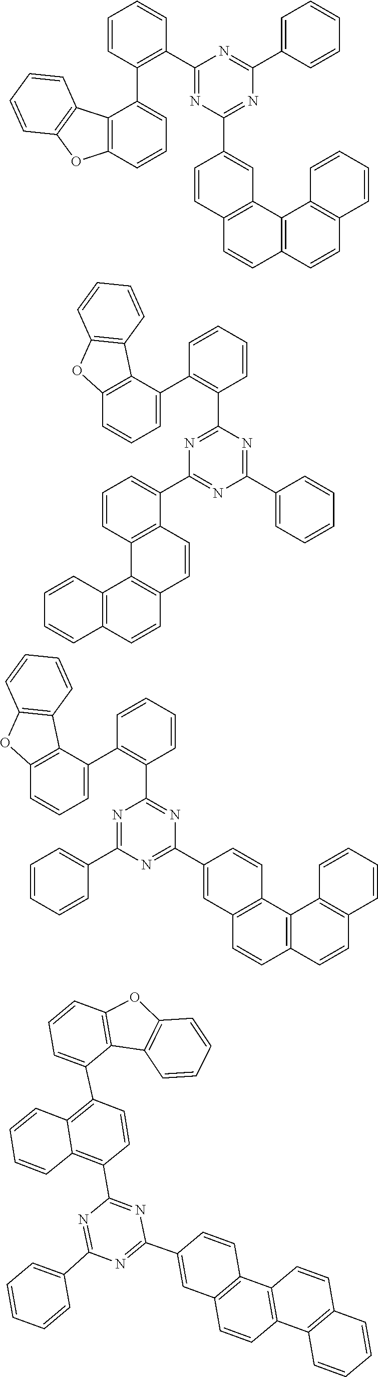

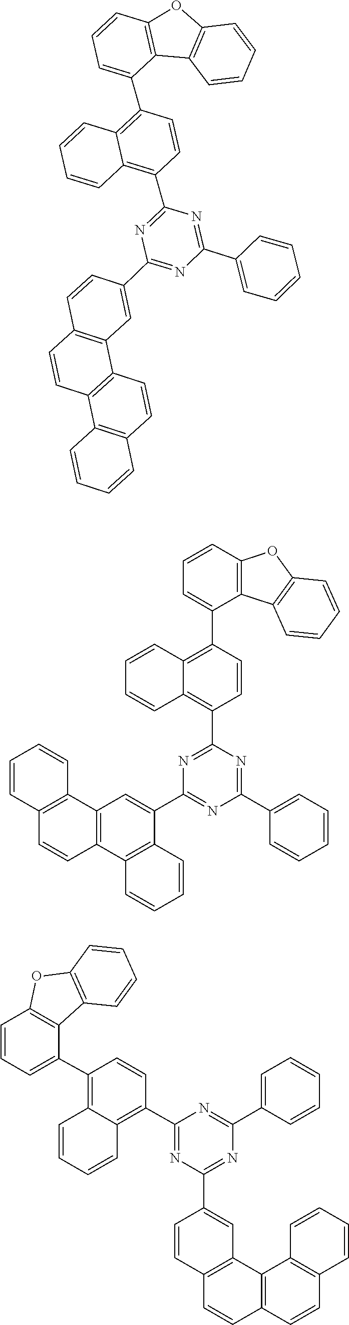 [SMILES: c1ccc(-c2nc(-c3ccc4ccc5c6ccccc6ccc5c4c3)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cc4c5ccccc5ccc4c4ccccc34)n2)cc1 ; c1ccc(-c2nc(-c3ccc4ccc5ccc6ccccc6c5c4c3)nc(-c3ccc(-c4cccc5oc6ccccc6c45)c4ccccc34)n2)cc1]

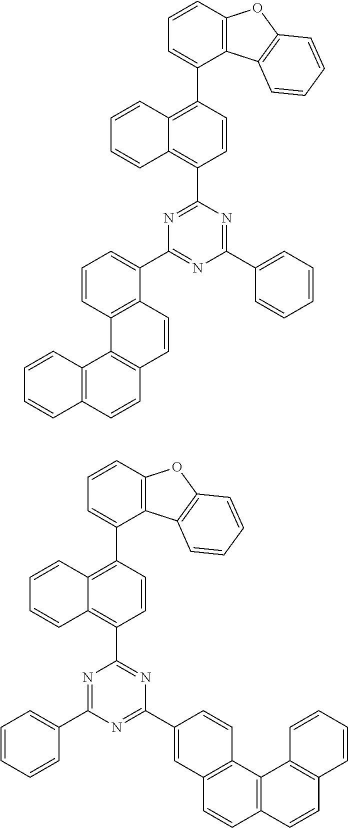

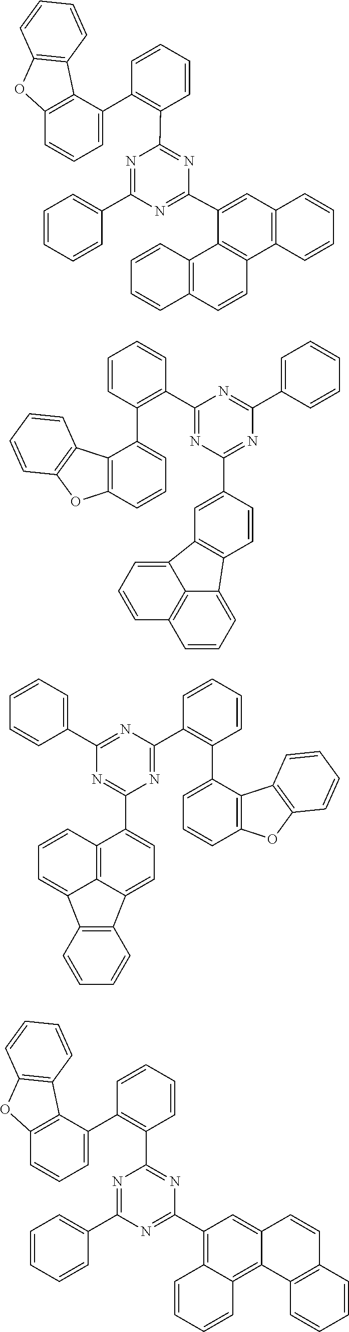

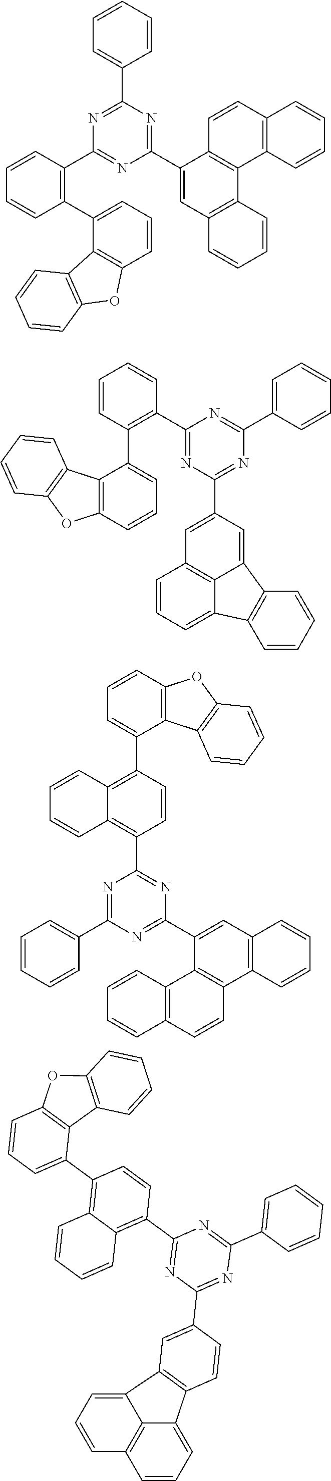

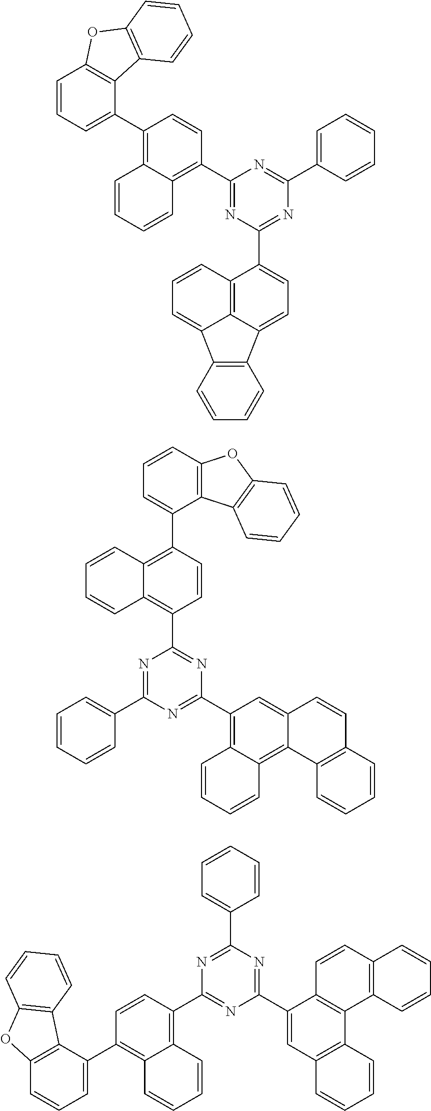

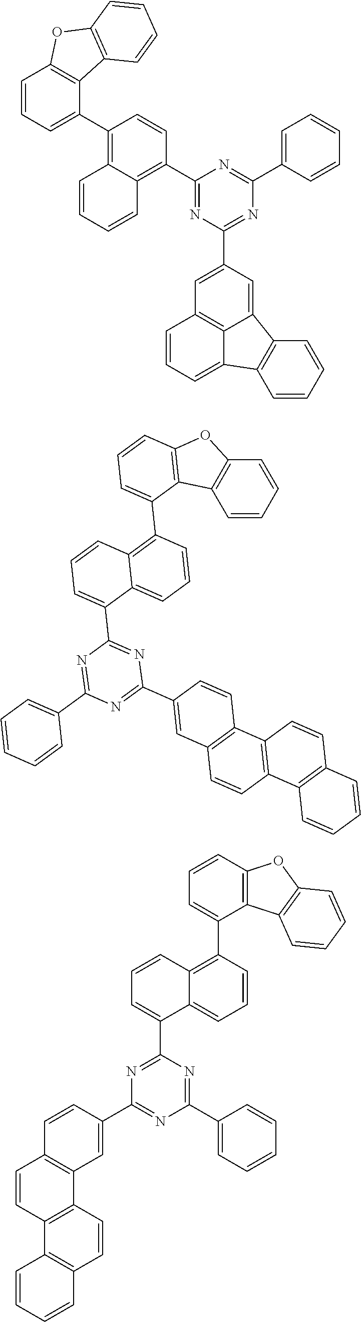 [SMILES: c1ccc(-c2nc(-c3cc4c5c(cccc5c3)-c3ccccc3-4)nc(-c3ccc(-c4cccc5oc6ccccc6c45)c4ccccc34)n2)cc1 ; c1ccc(-c2nc(-c3ccc4c(ccc5c6ccccc6ccc45)c3)nc(-c3cccc4c(-c5cccc6oc7ccccc7c56)cccc34)n2)cc1 ; c1ccc(-c2nc(-c3ccc4ccc5c6ccccc6ccc5c4c3)nc(-c3cccc4c(-c5cccc6oc7ccccc7c56)cccc34)n2)cc1]

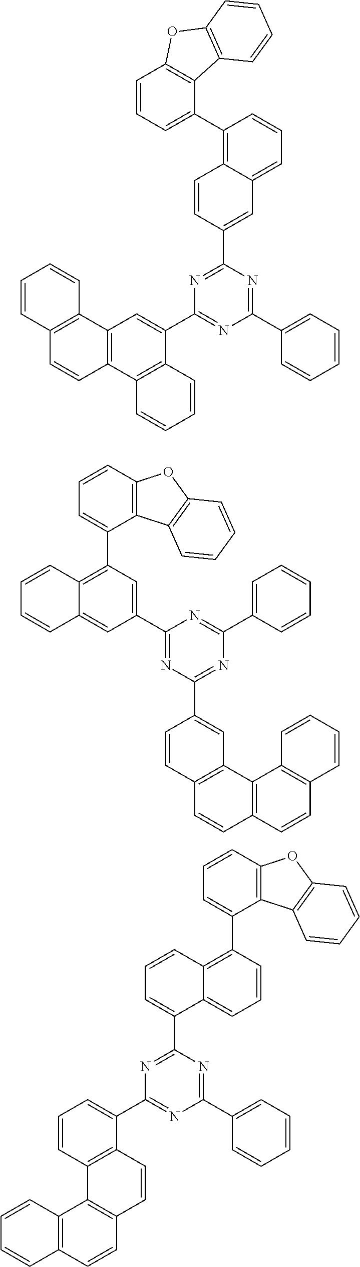

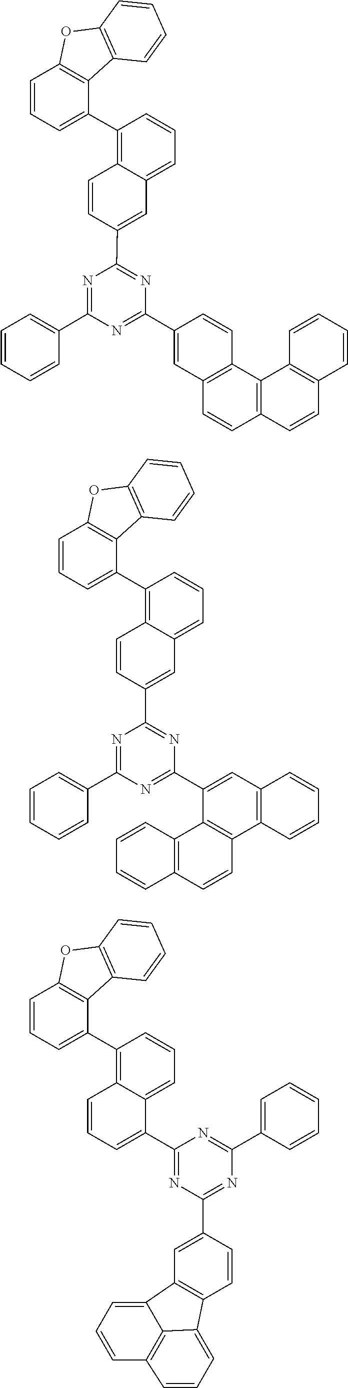

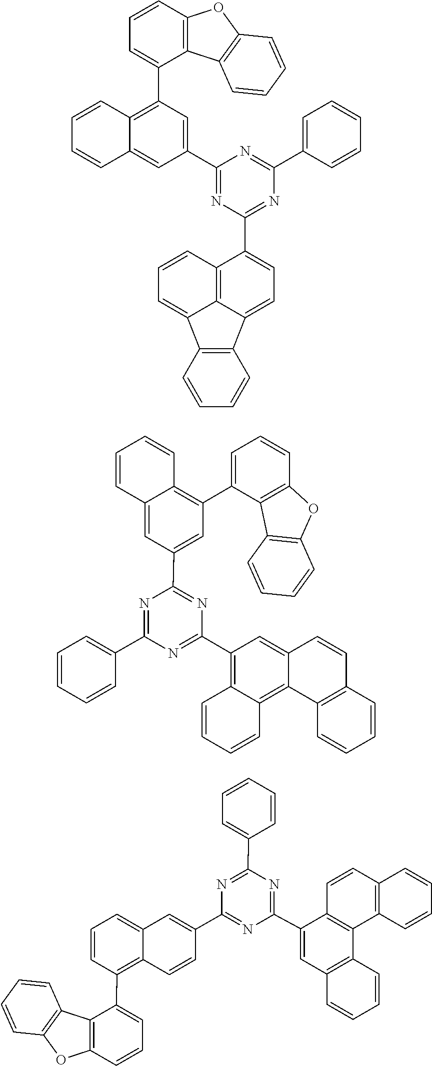

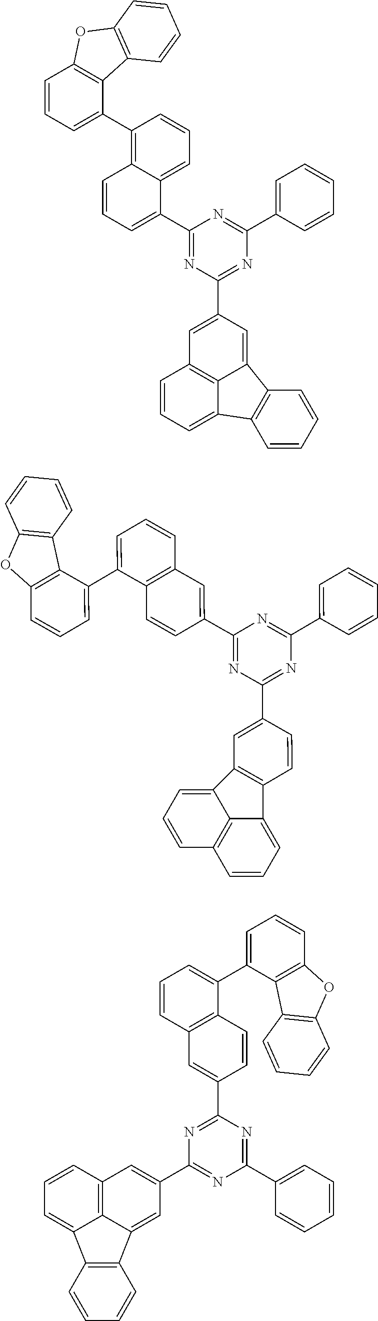

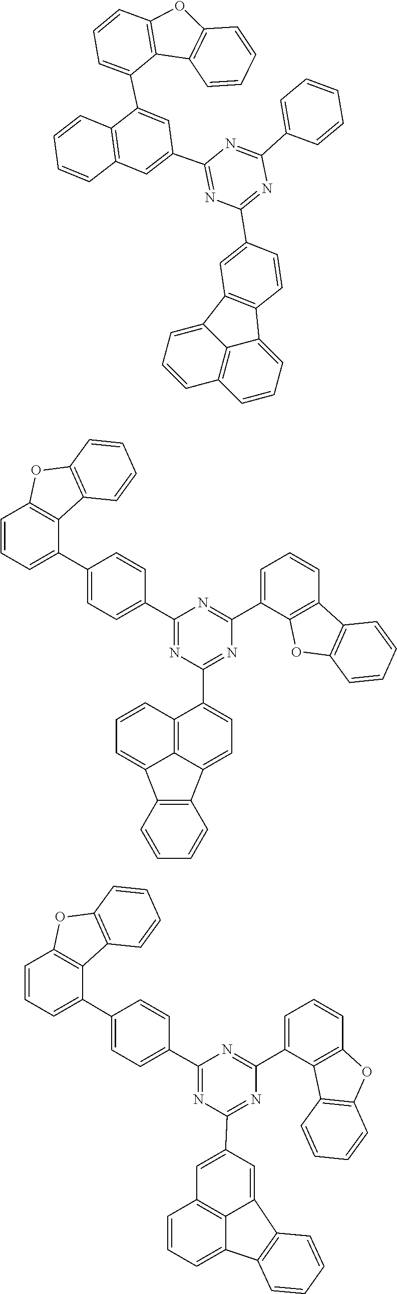

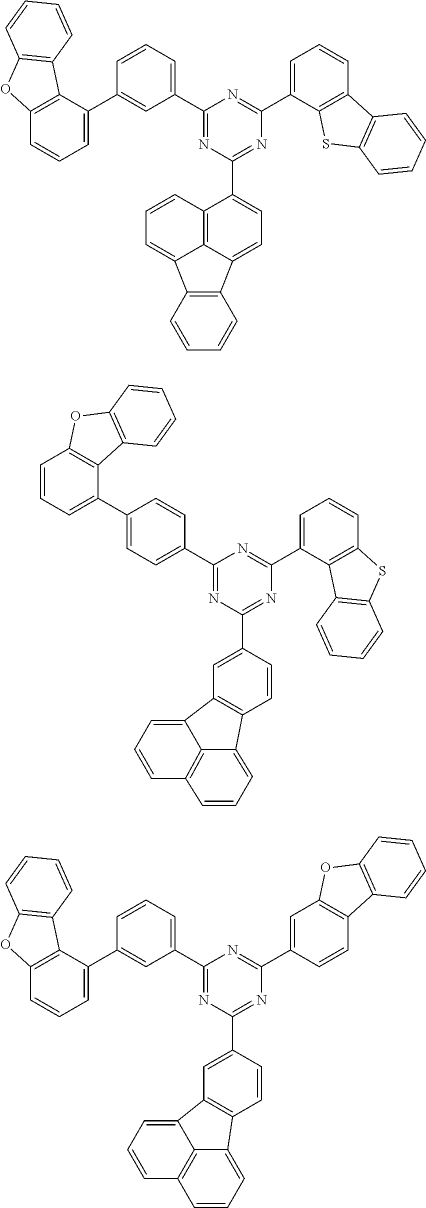

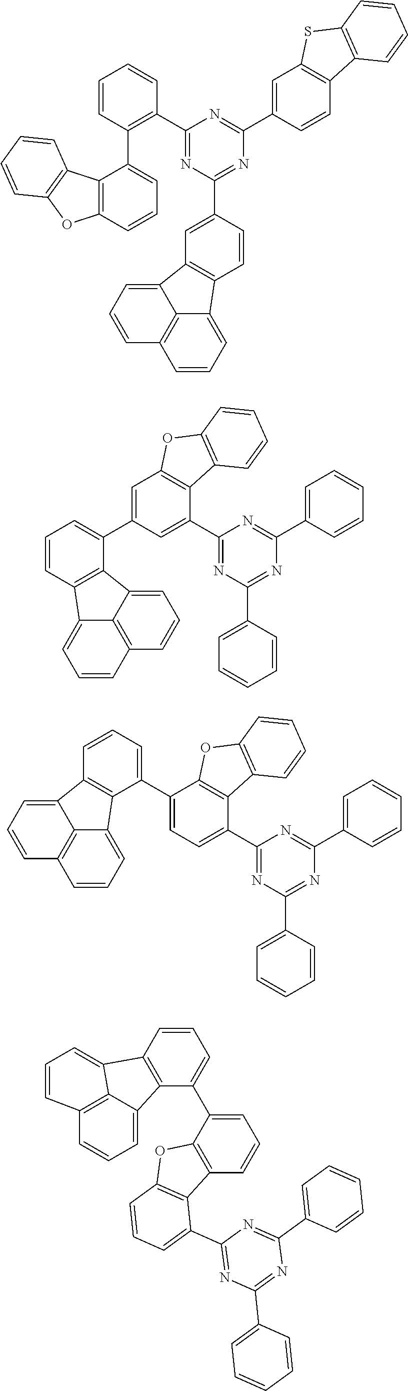

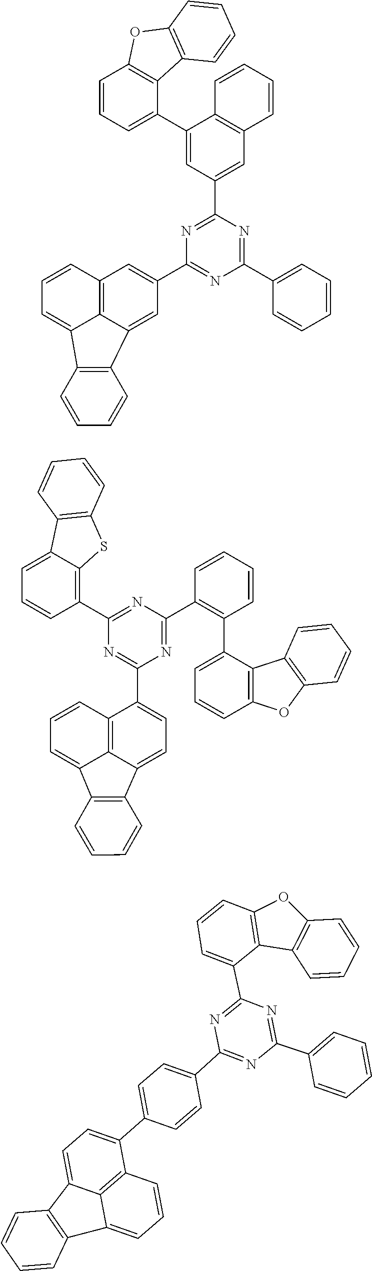

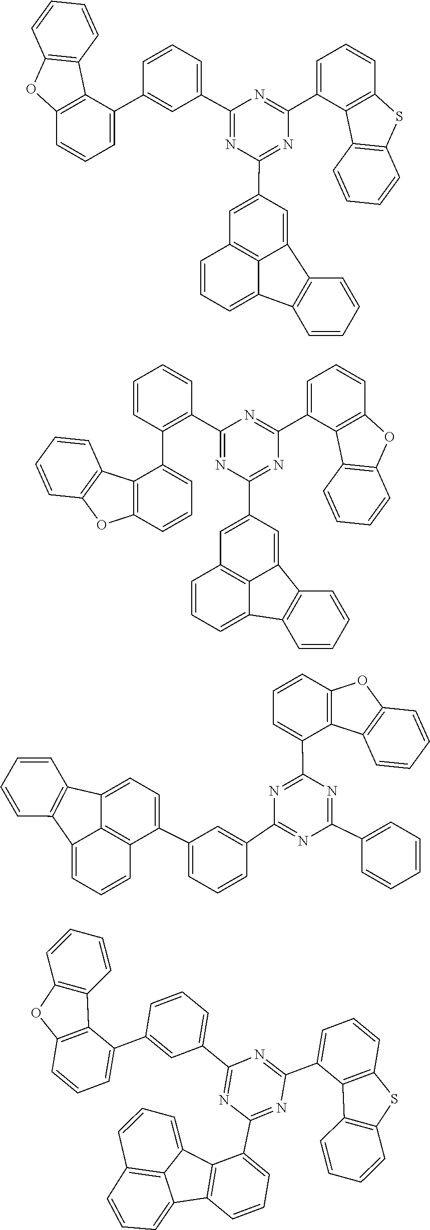

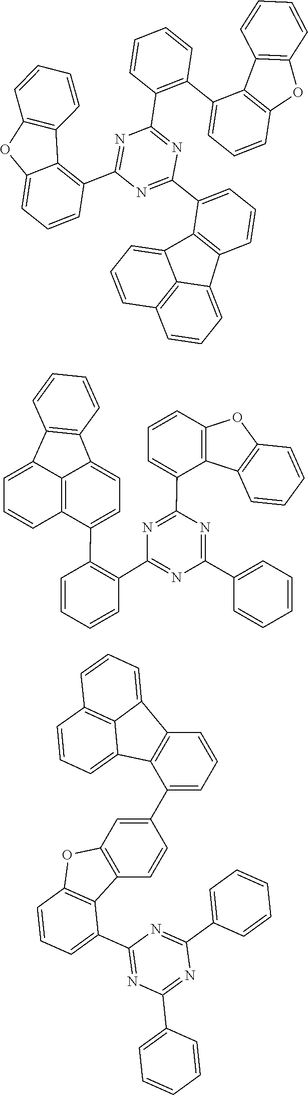

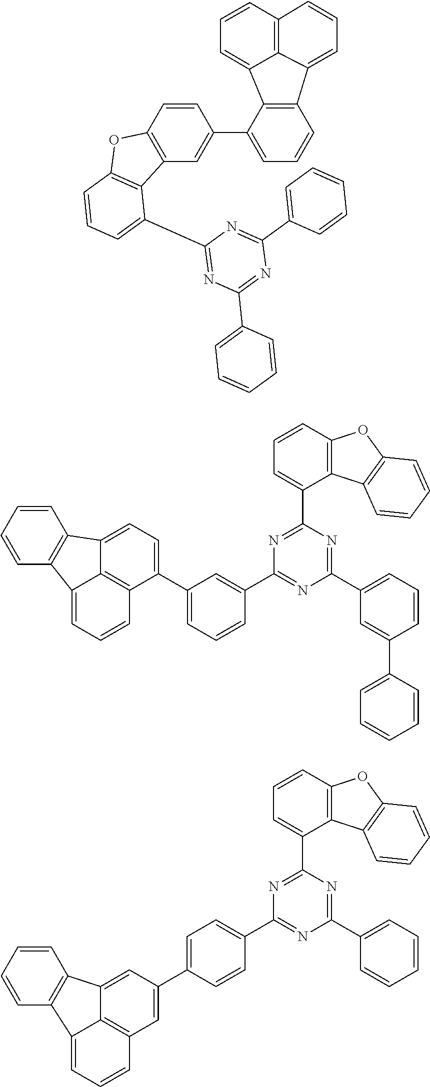

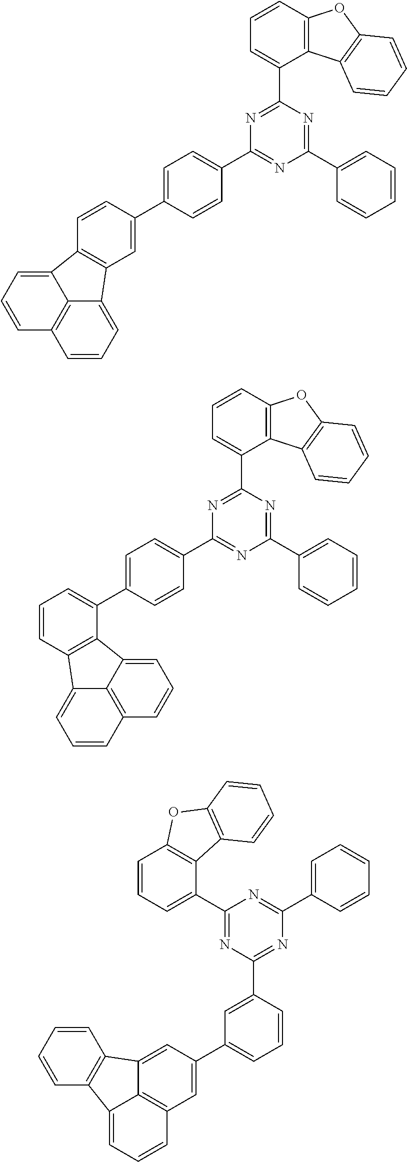 [SMILES: c1ccc(-c2nc(-c3ccc(-c4ccc5c(c4)-c4cccc6cccc-5c46)cc3)nc(-c3cccc4oc5ccccc5c34)n2)cc1 ; c1ccc(-c2nc(-c3ccc(-c4cccc5c4-c4cccc6cccc-5c46)cc3)nc(-c3cccc4oc5ccccc5c34)n2)cc1 ; c1ccc(-c2nc(-c3cccc(-c4cc5c6c(cccc6c4)-c4ccccc4-5)c3)nc(-c3cccc4oc5ccccc5c34)n2)cc1]

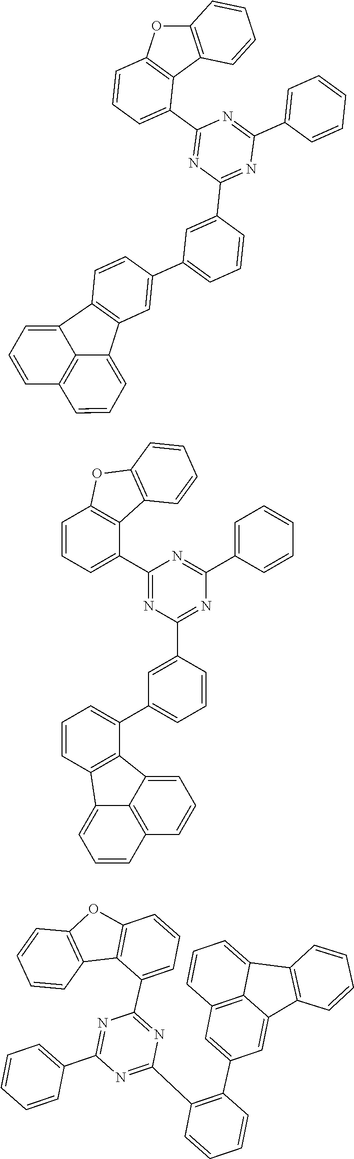

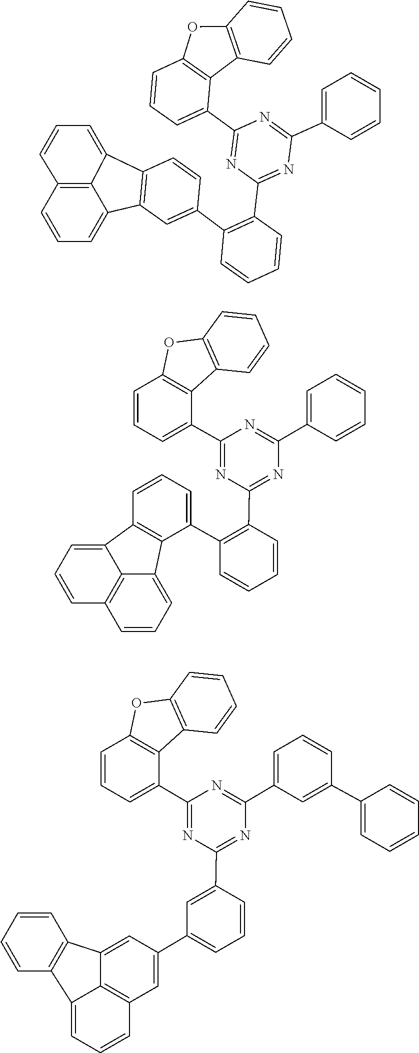

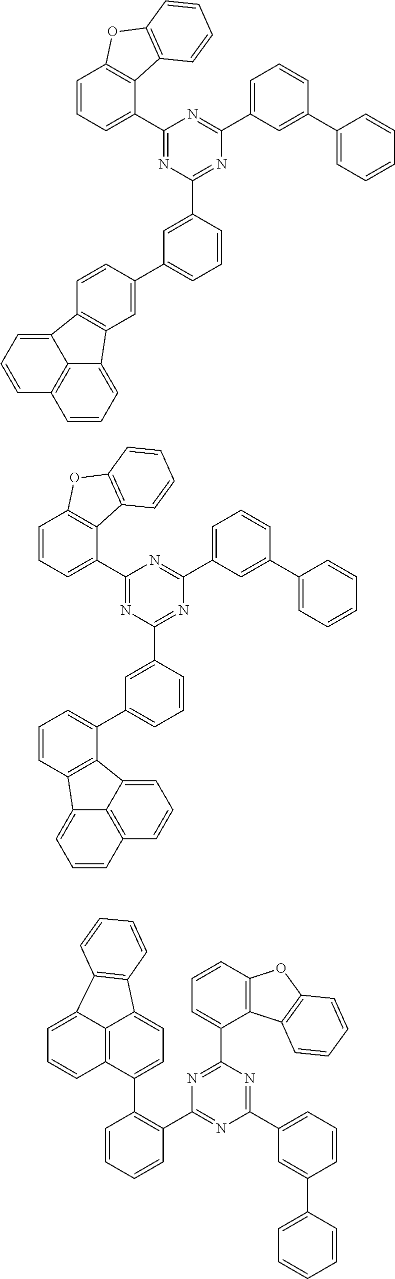

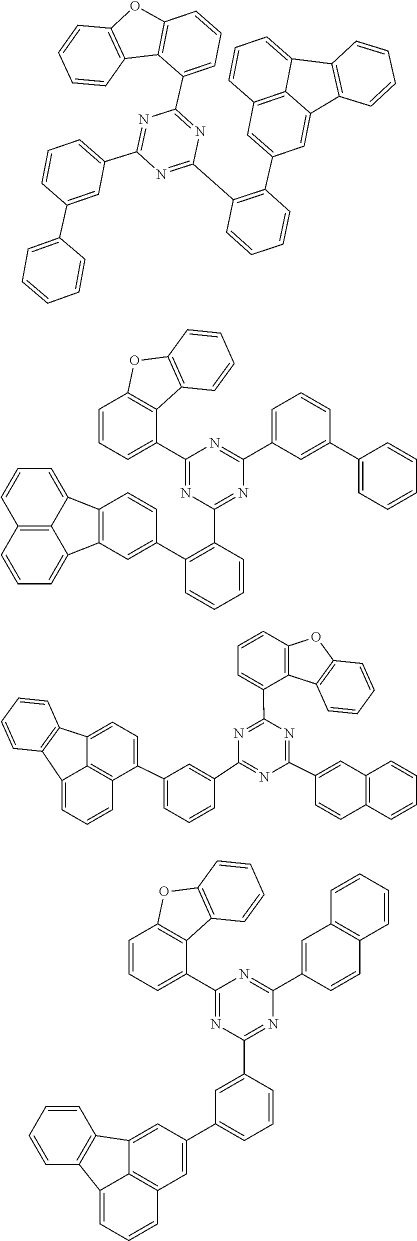

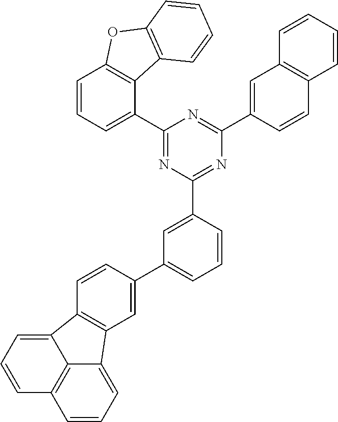 [SMILES: c1cc(-c2ccc3c(c2)-c2cccc4cccc-3c24)cc(-c2nc(-c3ccc4ccccc4c3)nc(-c3cccc4oc5ccccc5c34)n2)c1]

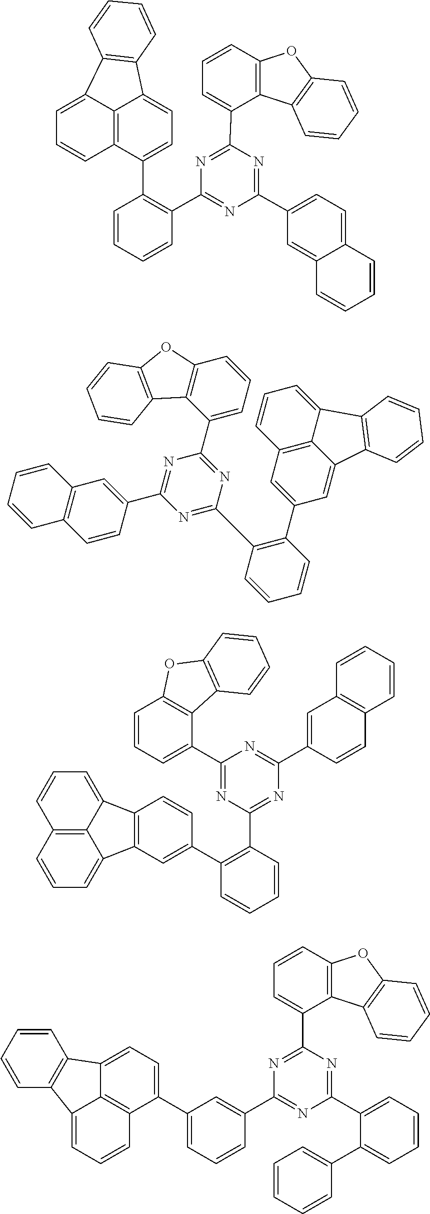

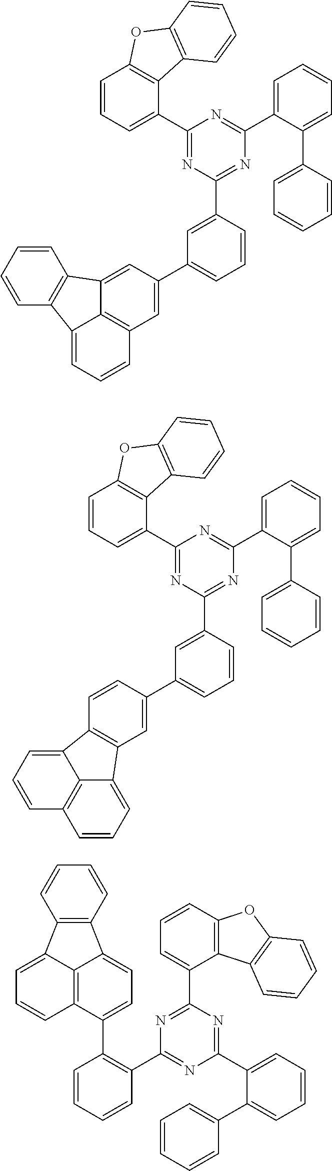

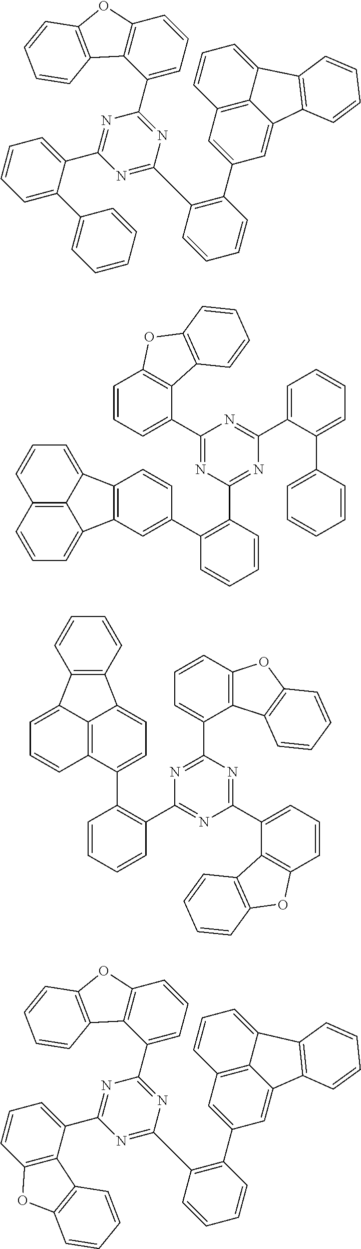

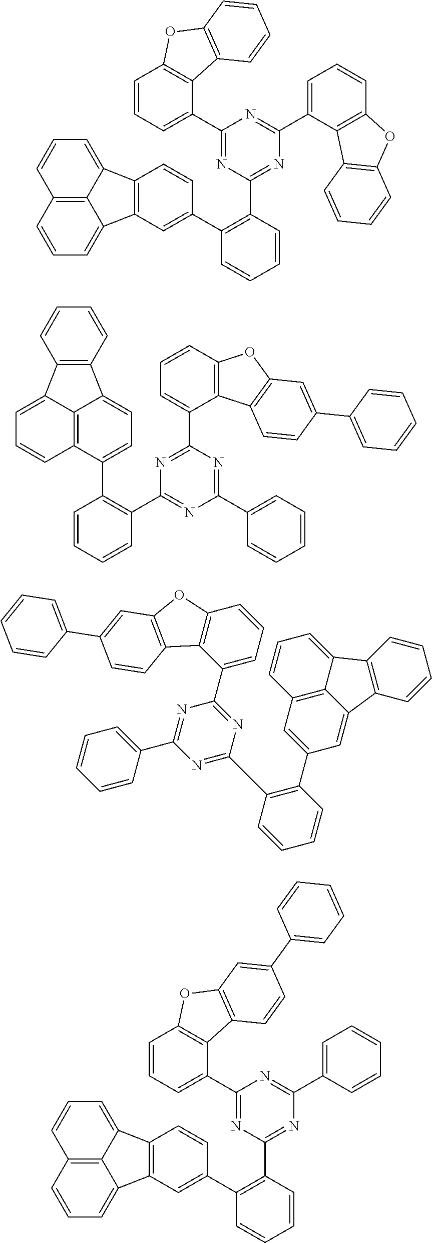

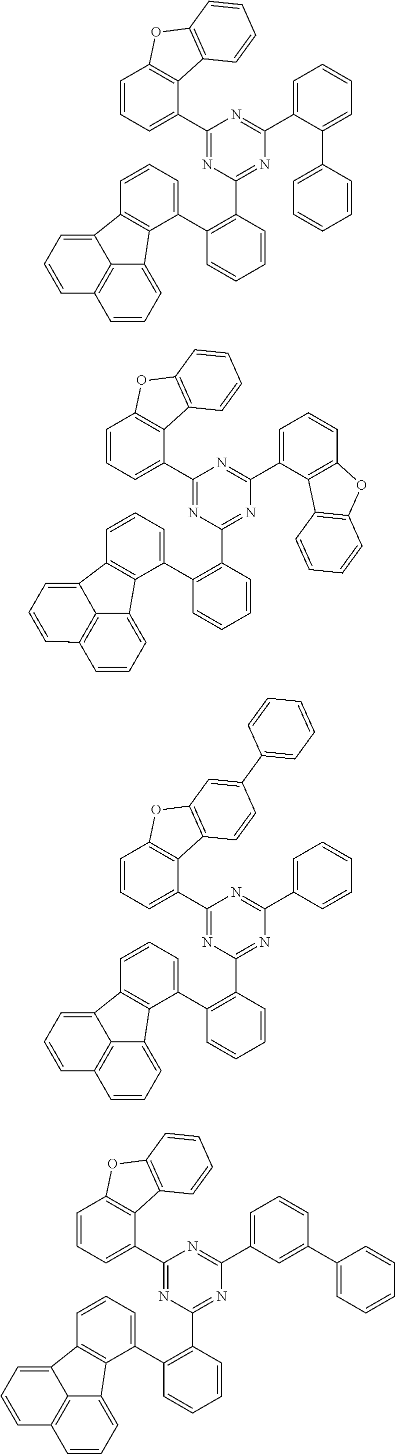

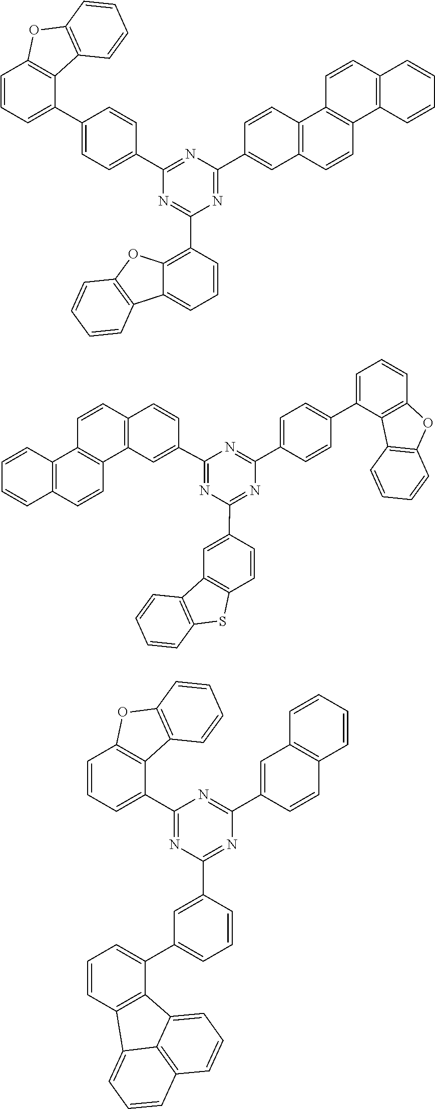

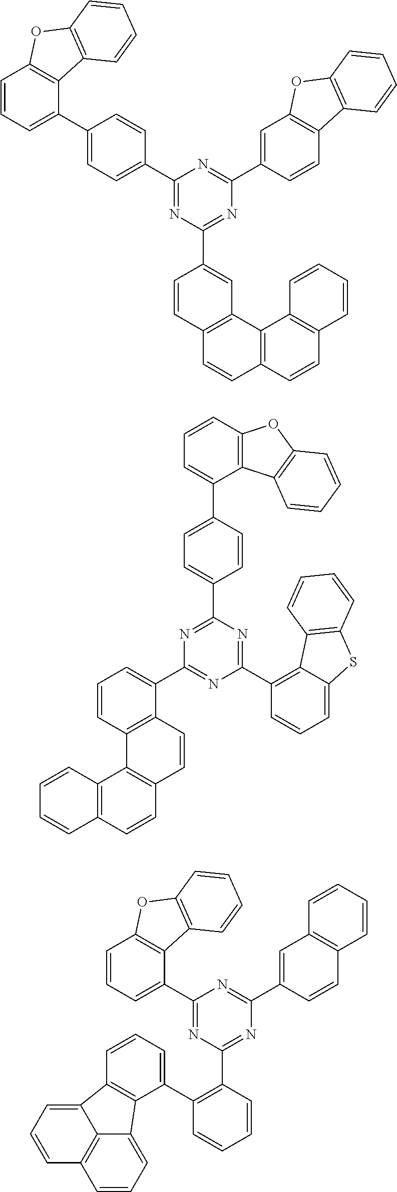

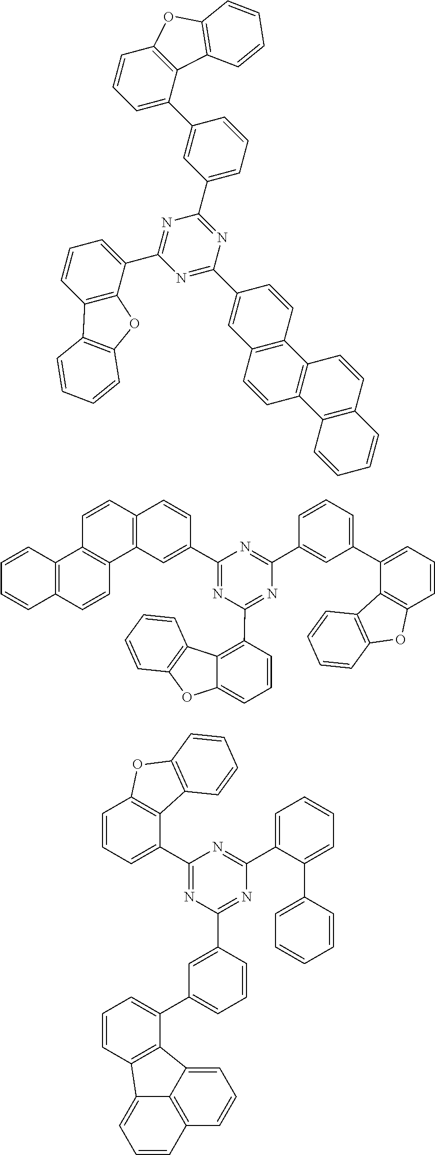

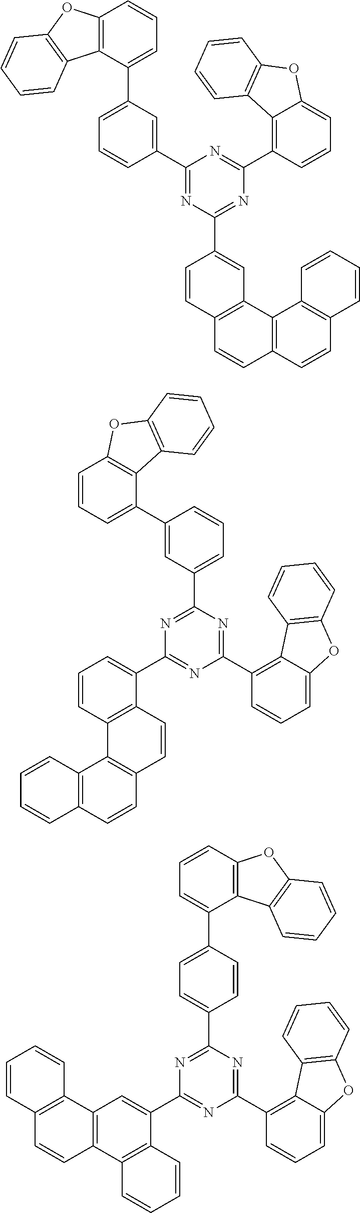 [SMILES: c1cc(-c2nc(-c3ccc4ccc5ccc6ccccc6c5c4c3)nc(-c3cccc4oc5ccccc5c34)n2)cc(-c2cccc3oc4ccccc4c23)c1 ; c1cc(-c2nc(-c3cccc4c3ccc3ccc5ccccc5c34)nc(-c3cccc4oc5ccccc5c34)n2)cc(-c2cccc3oc4ccccc4c23)c1 ; c1ccc2c(c1)ccc1c3ccccc3c(-c3nc(-c4ccc(-c5cccc6oc7ccccc7c56)cc4)nc(-c4cccc5oc6ccccc6c45)n3)cc21]

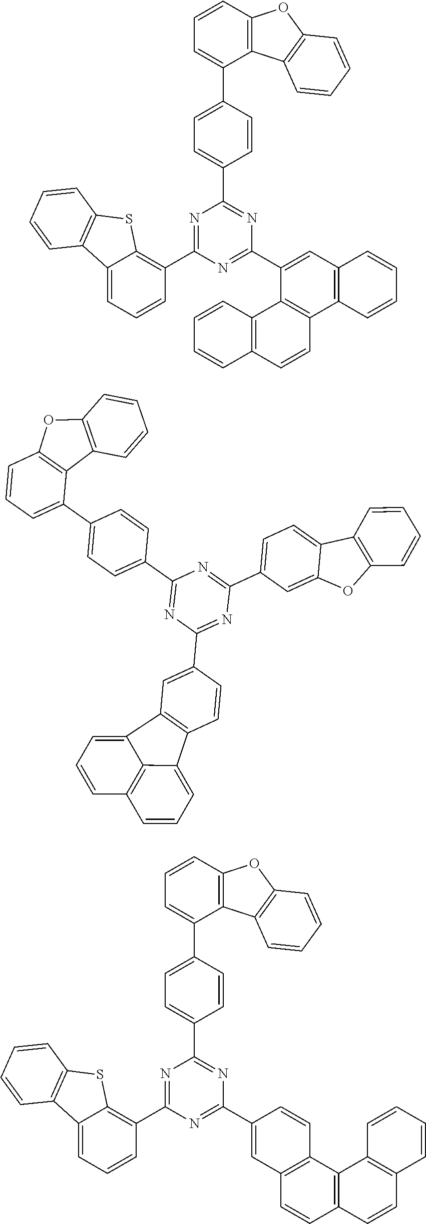

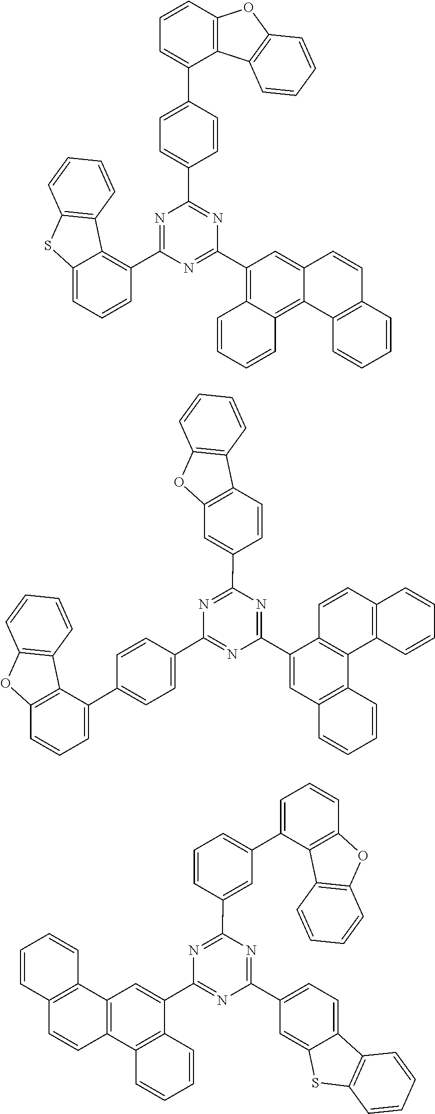

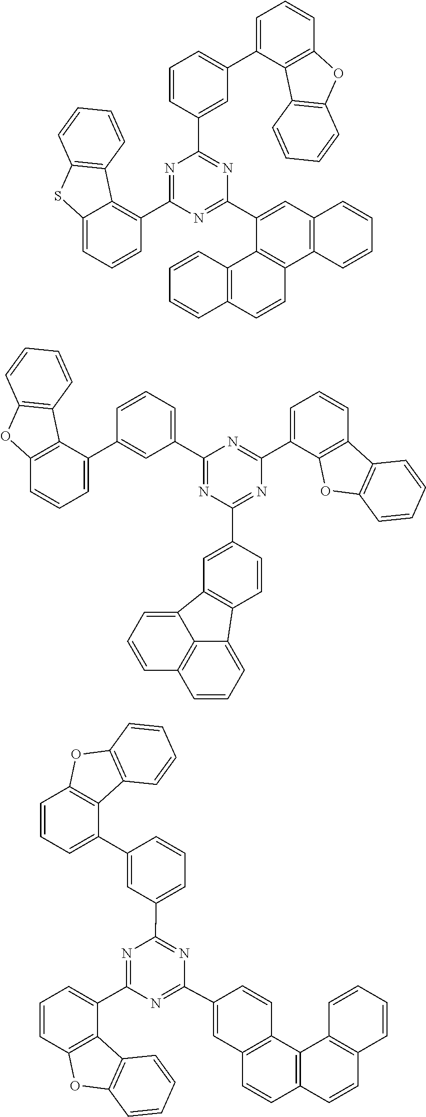

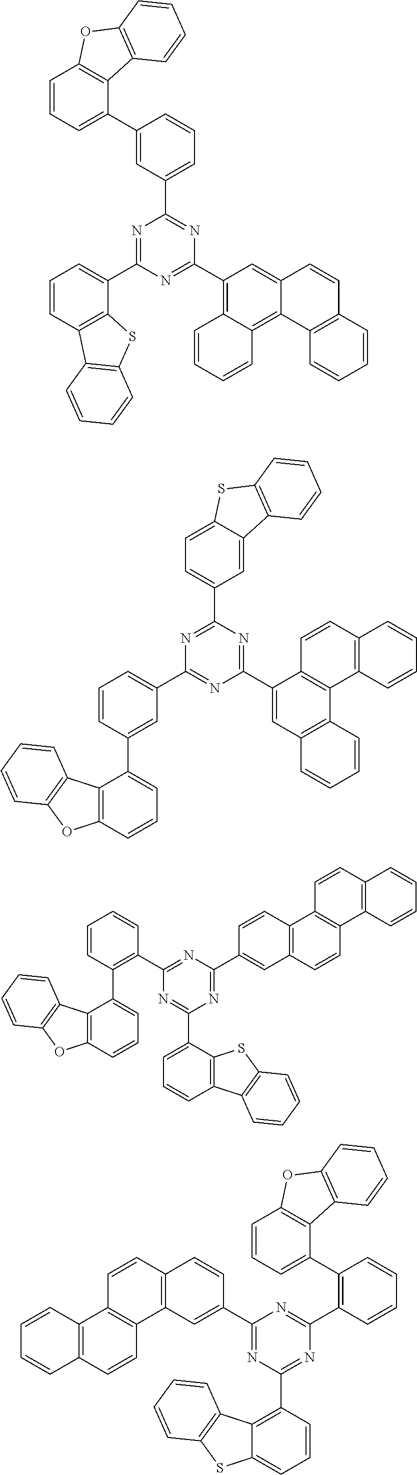 [SMILES: c1cc(-c2nc(-c3cc4ccc5ccccc5c4c4ccccc34)nc(-c3cccc4c3sc3ccccc34)n2)cc(-c2cccc3oc4ccccc4c23)c1 ; c1cc(-c2nc(-c3ccc4sc5ccccc5c4c3)nc(-c3cc4ccccc4c4c3ccc3ccccc34)n2)cc(-c2cccc3oc4ccccc4c23)c1 ; c1ccc(-c2cccc3oc4ccccc4c23)c(-c2nc(-c3ccc4c(ccc5c6ccccc6ccc45)c3)nc(-c3cccc4c3sc3ccccc34)n2)c1 ; c1ccc(-c2cccc3oc4ccccc4c23)c(-c2nc(-c3ccc4ccc5c6ccccc6ccc5c4c3)nc(-c3cccc4sc5ccccc5c34)n2)c1]

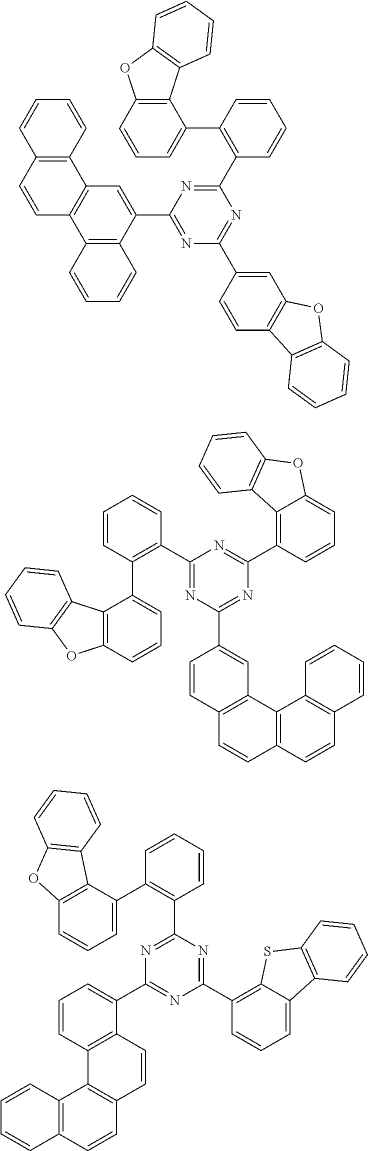 [SMILES: c1ccc(-c2cccc3oc4ccccc4c23)c(-c2nc(-c3ccc4c(c3)oc3ccccc34)nc(-c3cc4c5ccccc5ccc4c4ccccc34)n2)c1 ; c1ccc(-c2cccc3oc4ccccc4c23)c(-c2nc(-c3ccc4ccc5ccc6ccccc6c5c4c3)nc(-c3cccc4oc5ccccc5c34)n2)c1 ; c1ccc(-c2cccc3oc4ccccc4c23)c(-c2nc(-c3cccc4c3ccc3ccc5ccccc5c34)nc(-c3cccc4c3sc3ccccc34)n2)c1]

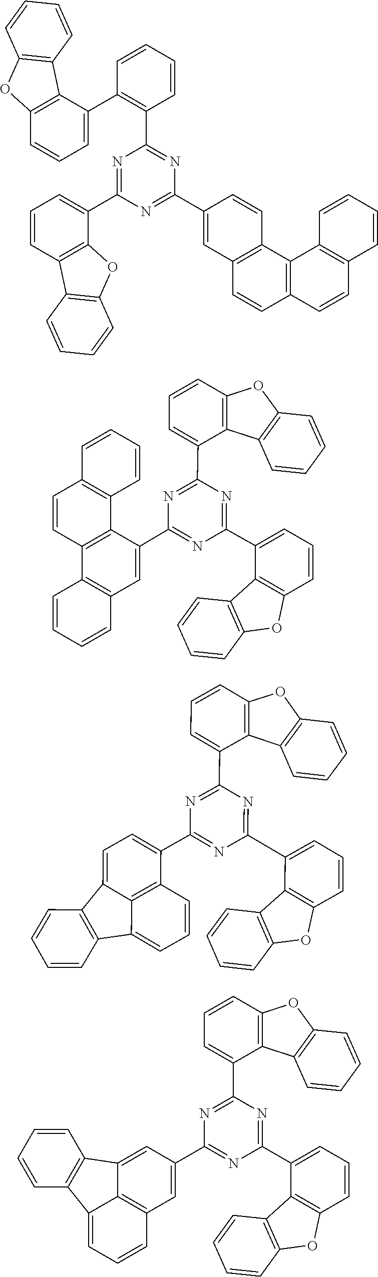

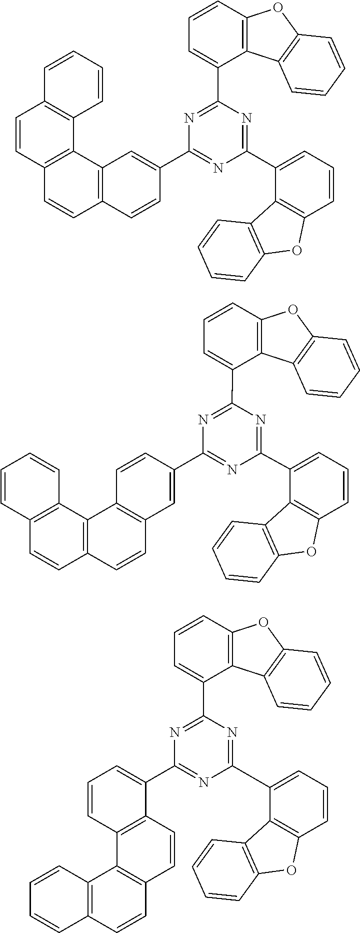

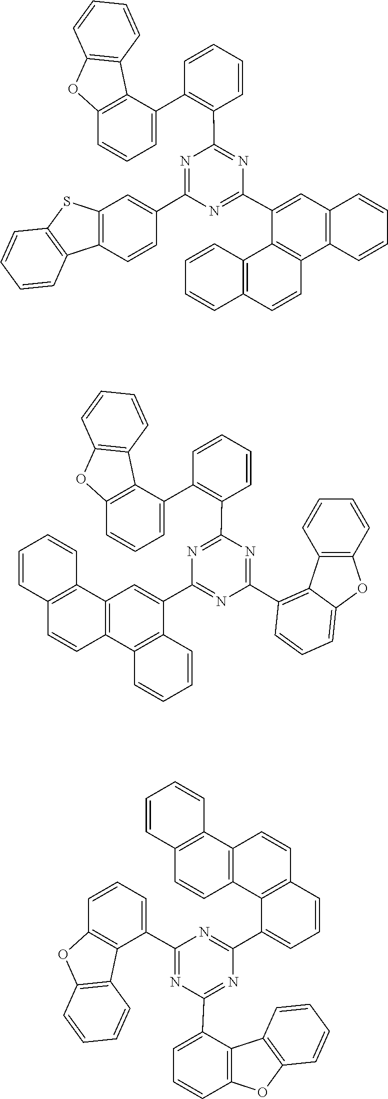 [SMILES: c1ccc(-c2cccc3oc4ccccc4c23)c(-c2nc(-c3ccc4c(c3)sc3ccccc34)nc(-c3cc4ccccc4c4ccc5ccccc5c34)n2)c1 ; c1ccc(-c2cccc3oc4ccccc4c23)c(-c2nc(-c3cc4c5ccccc5ccc4c4ccccc34)nc(-c3cccc4oc5ccccc5c34)n2)c1 ; c1ccc2c(c1)ccc1c2ccc2cccc(-c3nc(-c4cccc5oc6ccccc6c45)nc(-c4cccc5oc6ccccc6c45)n3)c21]

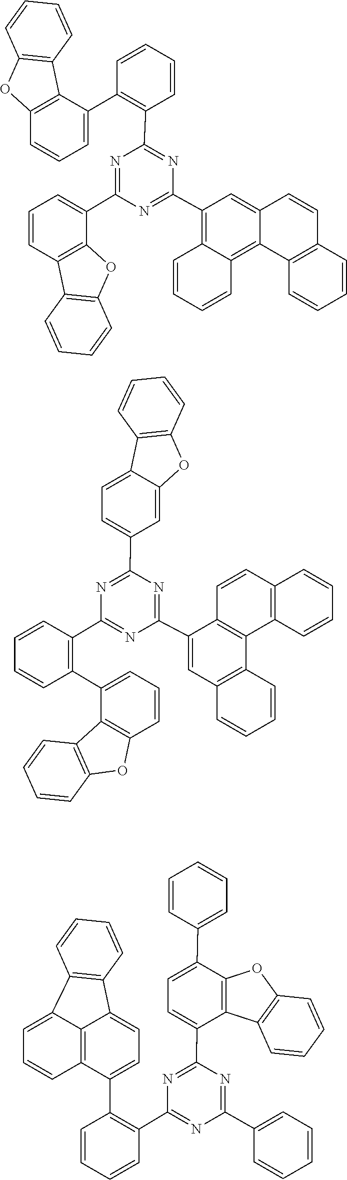 [SMILES: c1ccc(-c2cccc3oc4ccccc4c23)c(-c2nc(-c3cc4ccc5ccccc5c4c4ccccc34)nc(-c3cccc4c3oc3ccccc34)n2)c1 ; c1ccc(-c2cccc3oc4ccccc4c23)c(-c2nc(-c3ccc4c(c3)oc3ccccc34)nc(-c3cc4ccccc4c4c3ccc3ccccc34)n2)c1 ; c1ccc(-c2nc(-c3ccccc3-c3ccc4c5c(cccc35)-c3ccccc3-4)nc(-c3ccc(-c4ccccc4)c4oc5ccccc5c34)n2)cc1]

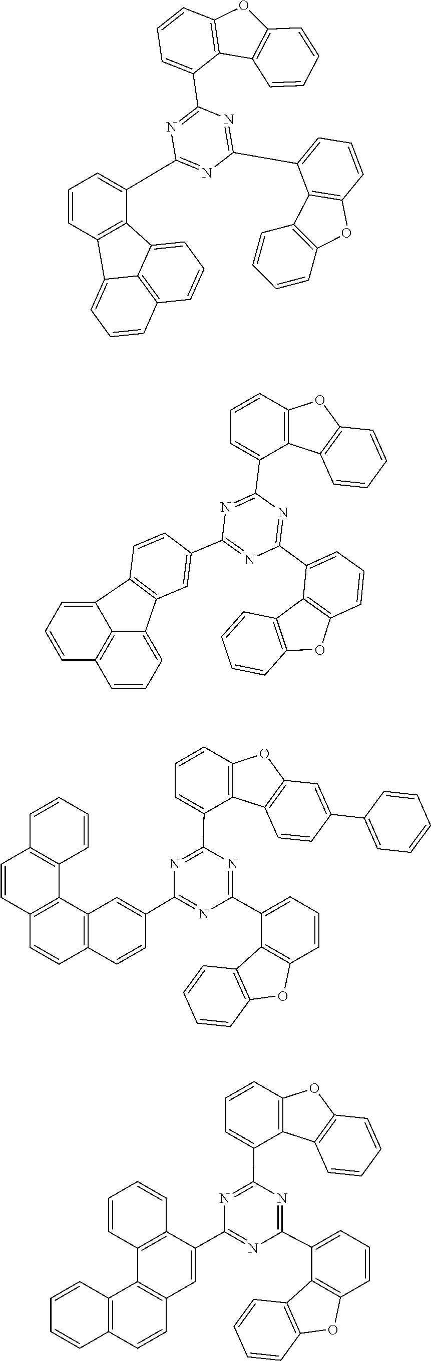 [SMILES: c1cc(-c2nc(-c3cccc4oc5ccccc5c34)nc(-c3cccc4oc5ccccc5c34)n2)c2c(c1)-c1cccc3cccc-2c13 ; c1cc2c3c(cccc3c1)-c1cc(-c3nc(-c4cccc5oc6ccccc6c45)nc(-c4cccc5oc6ccccc6c45)n3)ccc1-2 ; c1ccc(-c2ccc3c(c2)oc2cccc(-c4nc(-c5ccc6ccc7ccc8ccccc8c7c6c5)nc(-c5cccc6oc7ccccc7c56)n4)c23)cc1 ; c1ccc2c(c1)ccc1cc(-c3nc(-c4cccc5oc6ccccc6c45)nc(-c4cccc5oc6ccccc6c45)n3)c3ccccc3c12]

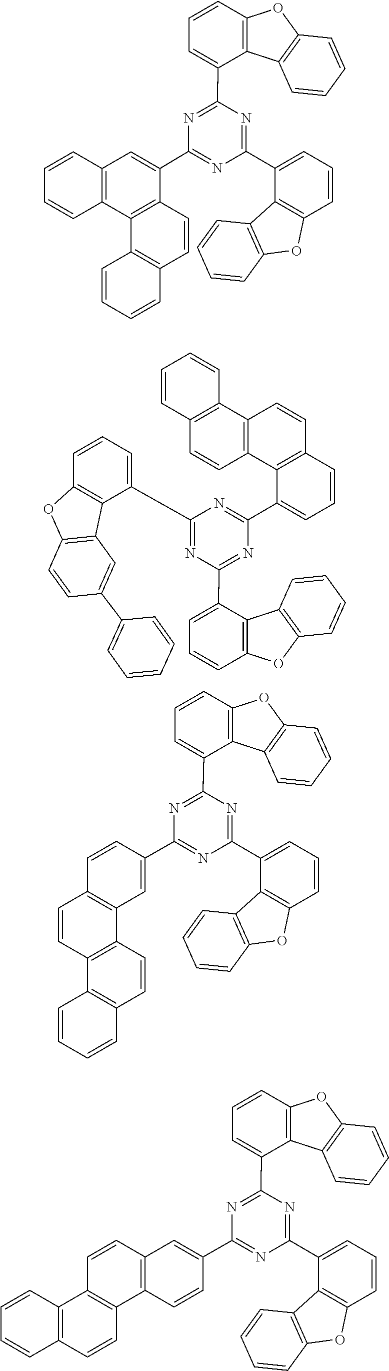

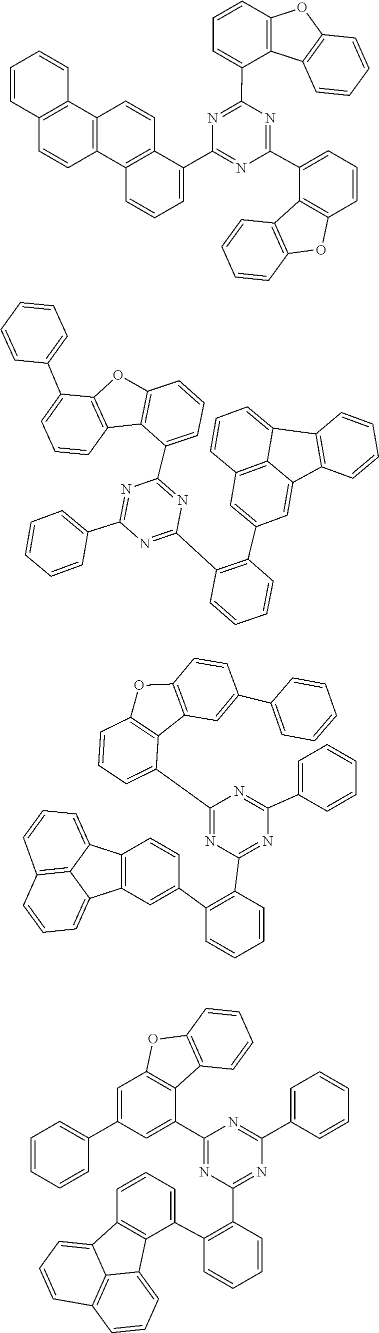

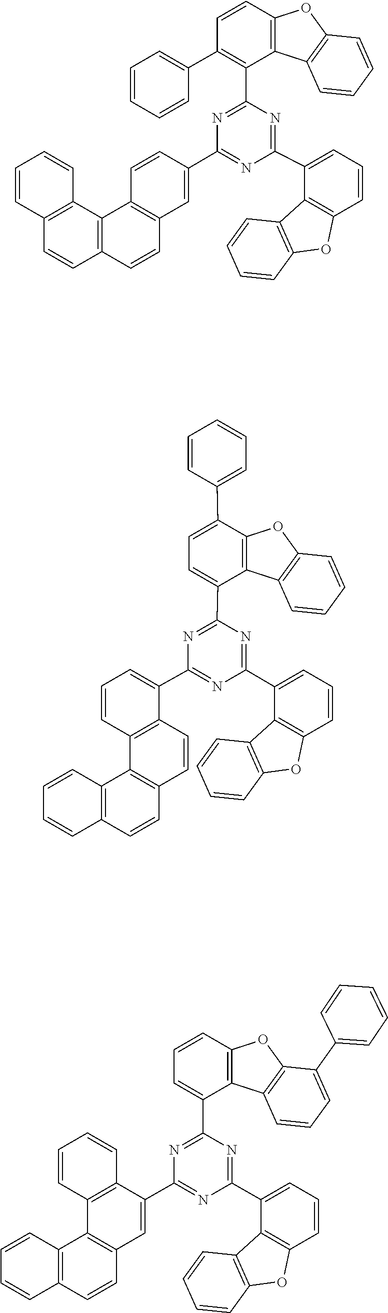 [SMILES: c1ccc(-c2ccc3oc4ccccc4c3c2-c2nc(-c3ccc4c(ccc5ccc6ccccc6c54)c3)nc(-c3cccc4oc5ccccc5c34)n2)cc1 ; c1ccc(-c2ccc(-c3nc(-c4cccc5c4ccc4ccc6ccccc6c45)nc(-c4cccc5oc6ccccc6c45)n3)c3c2oc2ccccc23)cc1 ; c1ccc(-c2cccc3c2oc2cccc(-c4nc(-c5cc6ccc7ccccc7c6c6ccccc56)nc(-c5cccc6oc7ccccc7c56)n4)c23)cc1]

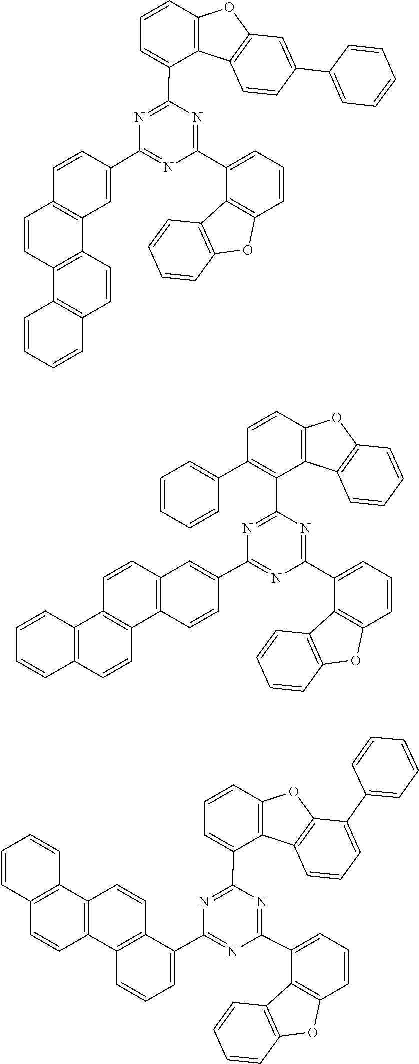 [SMILES: c1ccc(-c2ccc3c(c2)oc2cccc(-c4nc(-c5ccc6ccc7c8ccccc8ccc7c6c5)nc(-c5cccc6oc7ccccc7c56)n4)c23)cc1 ; c1ccc(-c2ccc3oc4ccccc4c3c2-c2nc(-c3ccc4c(ccc5c6ccccc6ccc45)c3)nc(-c3cccc4oc5ccccc5c34)n2)cc1 ; c1ccc(-c2cccc3c2oc2cccc(-c4nc(-c5cccc6c5ccc5c7ccccc7ccc65)nc(-c5cccc6oc7ccccc7c56)n4)c23)cc1]

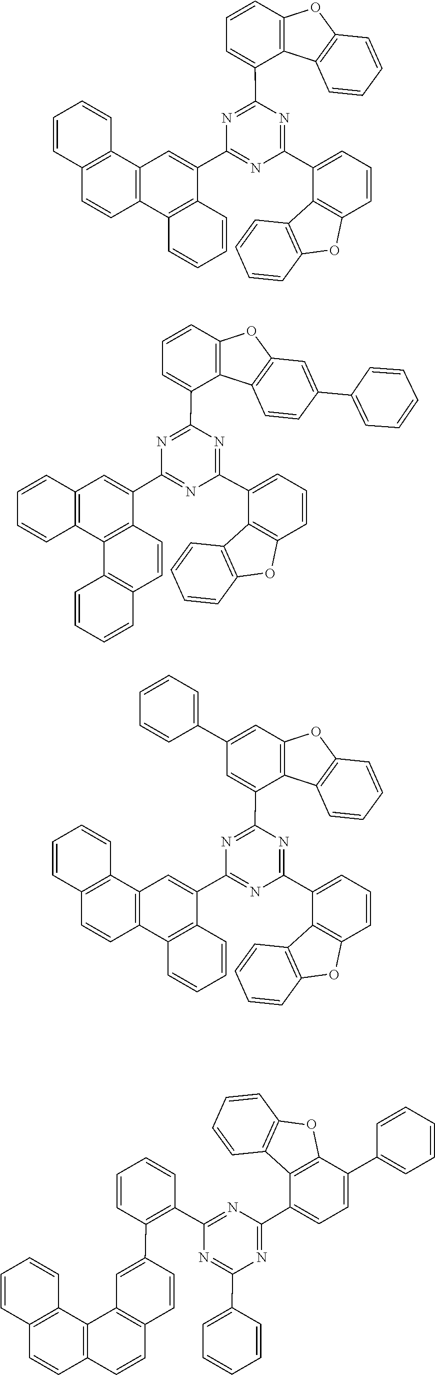 [SMILES: c1ccc2c(c1)ccc1c3ccccc3c(-c3nc(-c4cccc5oc6ccccc6c45)nc(-c4cccc5oc6ccccc6c45)n3)cc21 ; c1ccc(-c2ccc3c(c2)oc2cccc(-c4nc(-c5cc6ccccc6c6c5ccc5ccccc56)nc(-c5cccc6oc7ccccc7c56)n4)c23)cc1 ; c1ccc(-c2cc(-c3nc(-c4cc5c6ccccc6ccc5c5ccccc45)nc(-c4cccc5oc6ccccc6c45)n3)c3c(c2)oc2ccccc23)cc1 ; c1ccc(-c2nc(-c3ccccc3-c3ccc4ccc5ccc6ccccc6c5c4c3)nc(-c3ccc(-c4ccccc4)c4oc5ccccc5c34)n2)cc1]

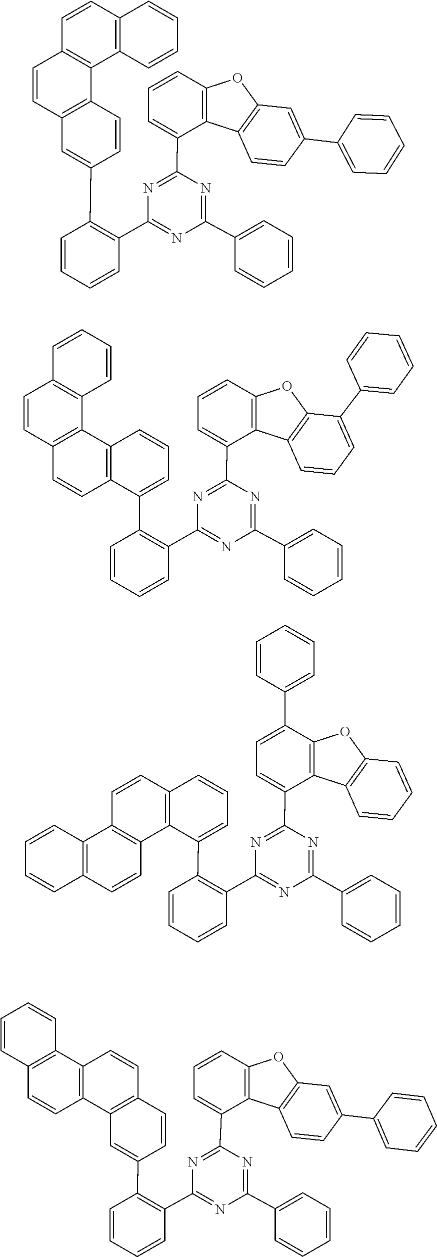

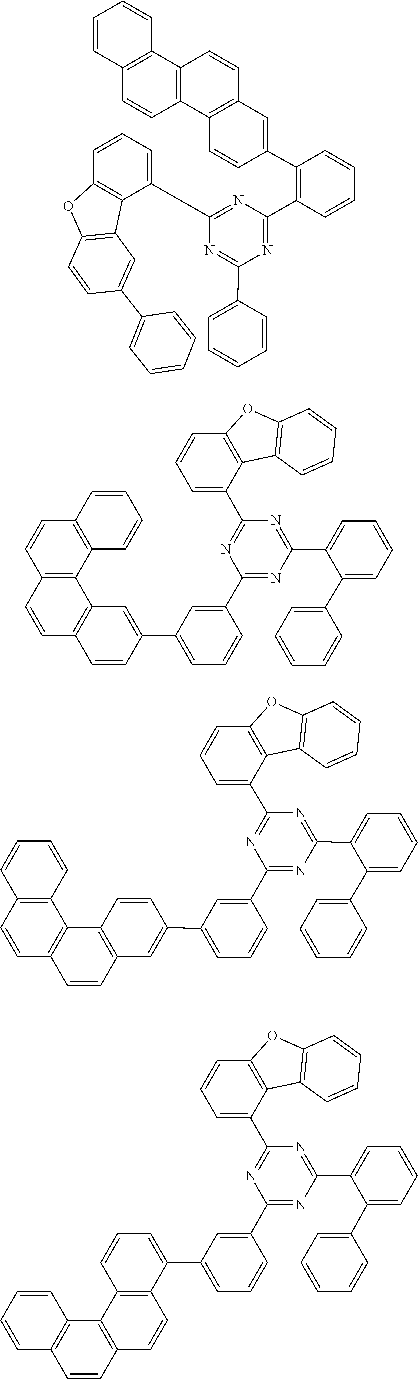

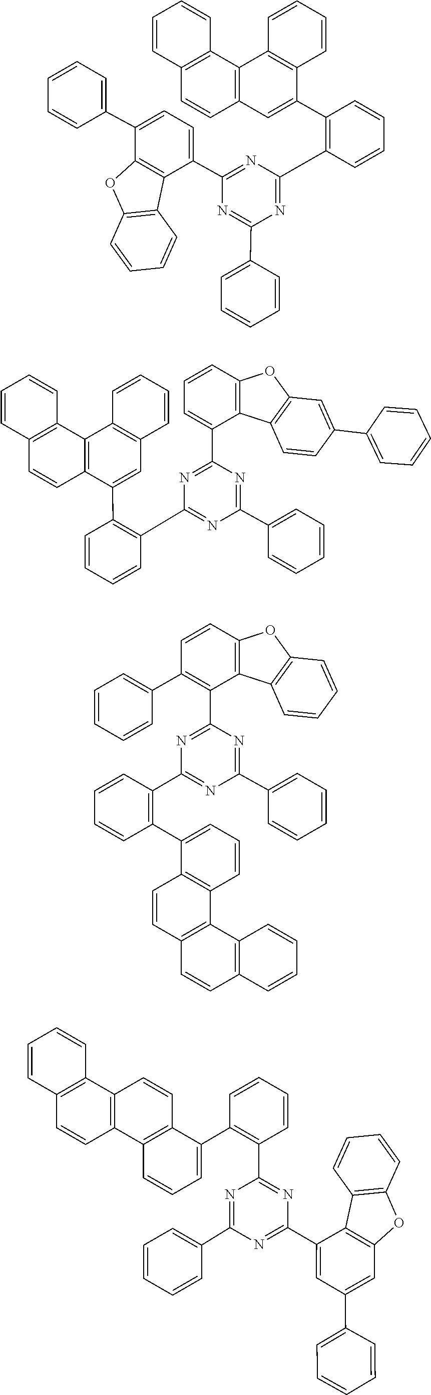 [SMILES: c1ccc(-c2nc(-c3ccccc3-c3cc4ccc5ccccc5c4c4ccccc34)nc(-c3ccc(-c4ccccc4)c4oc5ccccc5c34)n2)cc1 ; c1ccc(-c2ccc3c(c2)oc2cccc(-c4nc(-c5ccccc5)nc(-c5ccccc5-c5cc6ccccc6c6c5ccc5ccccc56)n4)c23)cc1 ; c1ccc(-c2nc(-c3ccccc3-c3cccc4c3ccc3ccc5ccccc5c34)nc(-c3c(-c4ccccc4)ccc4oc5ccccc5c34)n2)cc1 ; c1ccc(-c2cc(-c3nc(-c4ccccc4)nc(-c4ccccc4-c4cccc5c4ccc4c6ccccc6ccc54)n3)c3c(c2)oc2ccccc23)cc1]

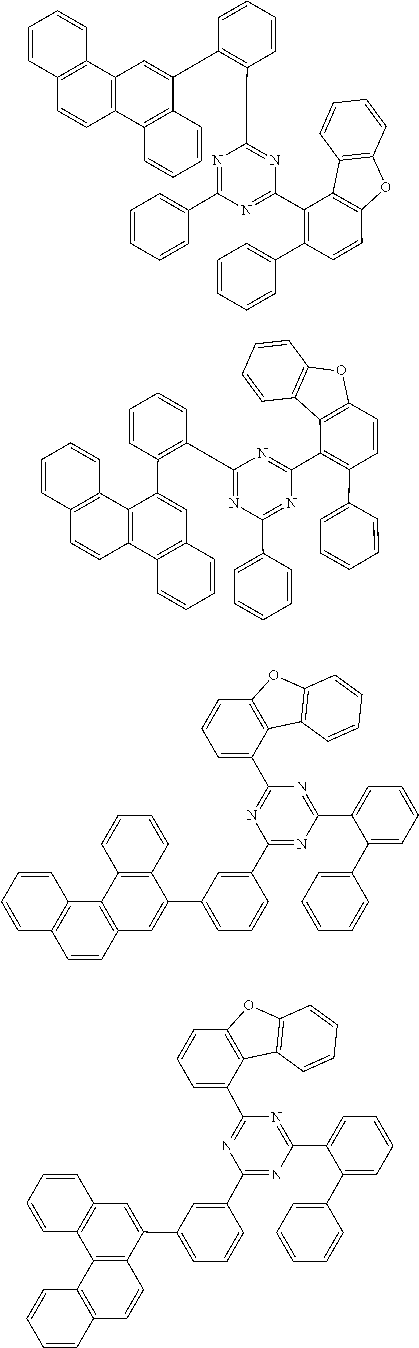

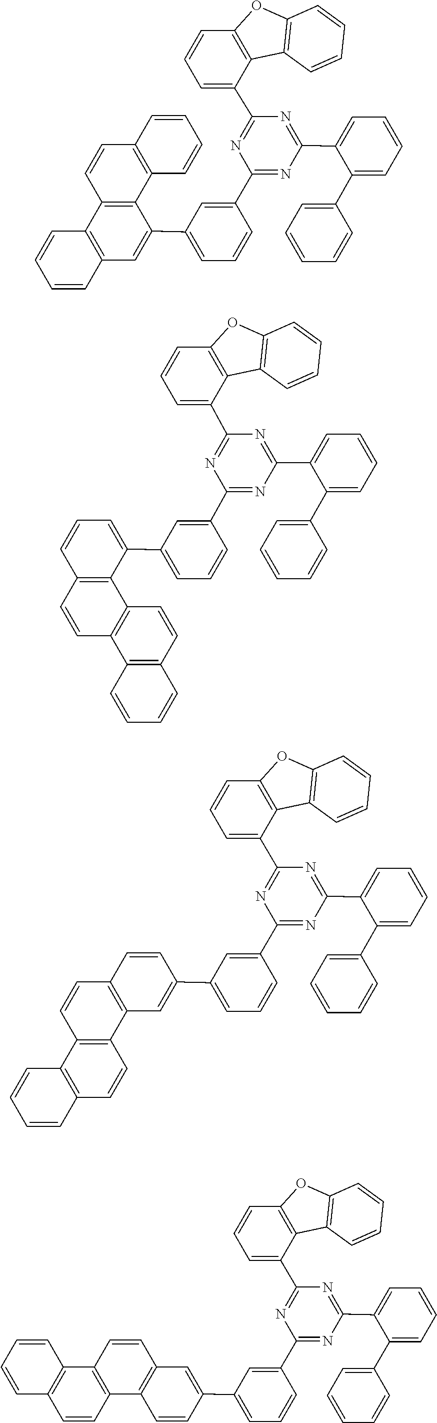 [SMILES: c1ccc(-c2ccccc2-c2nc(-c3cccc(-c4cc5ccccc5c5ccc6ccccc6c45)c3)nc(-c3cccc4oc5ccccc5c34)n2)cc1 ; c1ccc(-c2ccccc2-c2nc(-c3cccc(-c4cccc5ccc6c7ccccc7ccc6c45)c3)nc(-c3cccc4oc5ccccc5c34)n2)cc1 ; c1ccc(-c2ccccc2-c2nc(-c3cccc(-c4ccc5ccc6c7ccccc7ccc6c5c4)c3)nc(-c3cccc4oc5ccccc5c34)n2)cc1 ; c1ccc(-c2ccccc2-c2nc(-c3cccc(-c4ccc5c(ccc6c7ccccc7ccc56)c4)c3)nc(-c3cccc4oc5ccccc5c34)n2)cc1]

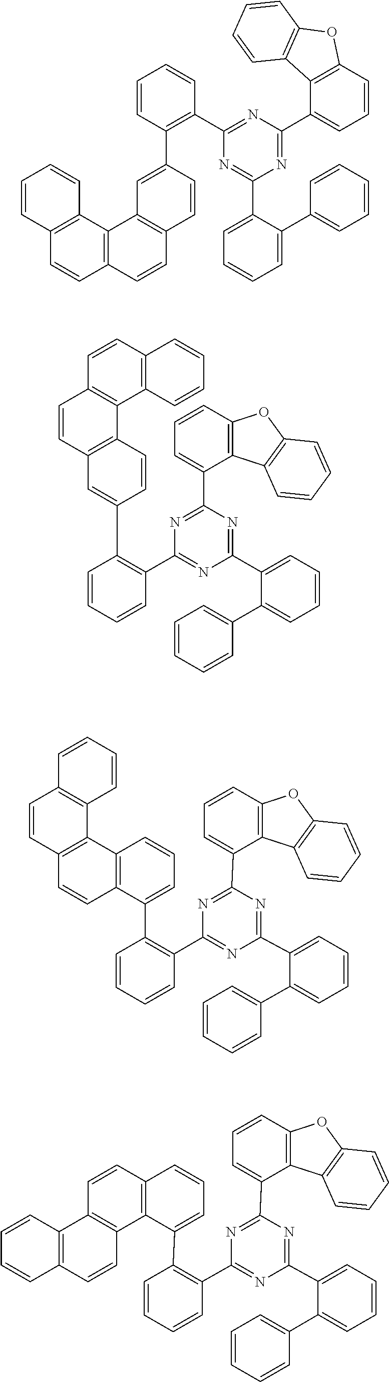 [SMILES: c1ccc(-c2ccccc2-c2nc(-c3ccccc3-c3ccc4ccc5ccc6ccccc6c5c4c3)nc(-c3cccc4oc5ccccc5c34)n2)cc1 ; c1ccc(-c2ccccc2-c2nc(-c3ccccc3-c3ccc4c(ccc5ccc6ccccc6c54)c3)nc(-c3cccc4oc5ccccc5c34)n2)cc1 ; c1ccc(-c2ccccc2-c2nc(-c3ccccc3-c3cccc4c3ccc3ccc5ccccc5c34)nc(-c3cccc4oc5ccccc5c34)n2)cc1 ; c1ccc(-c2ccccc2-c2nc(-c3ccccc3-c3cccc4ccc5c6ccccc6ccc5c34)nc(-c3cccc4oc5ccccc5c34)n2)cc1]

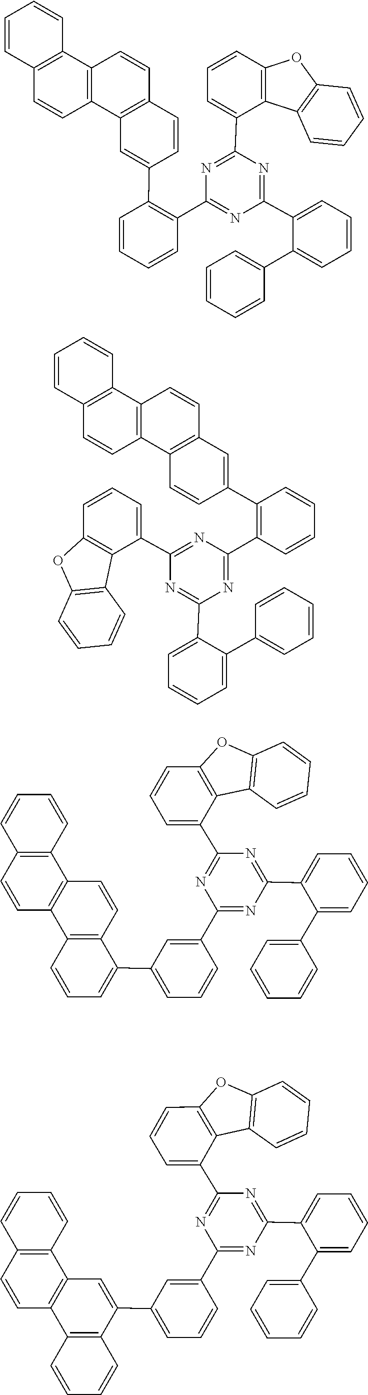

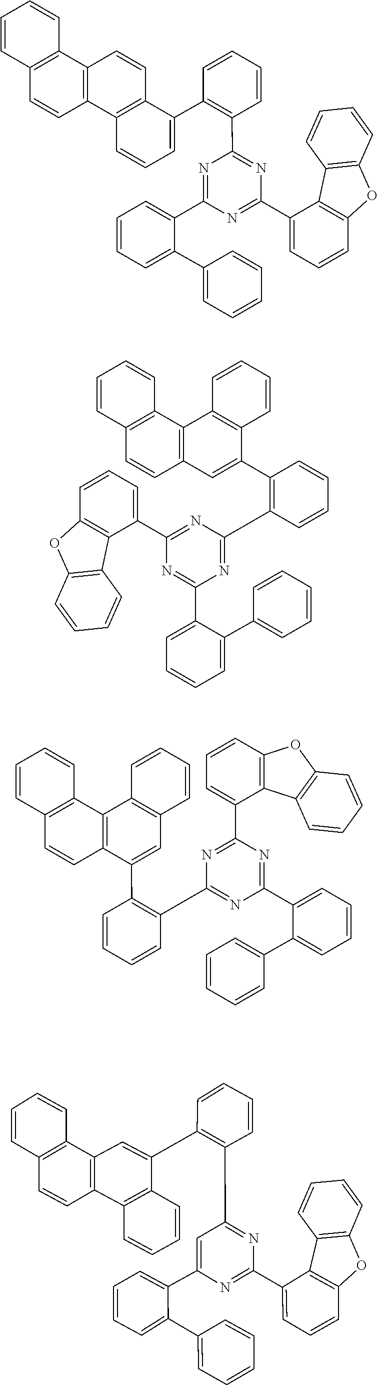

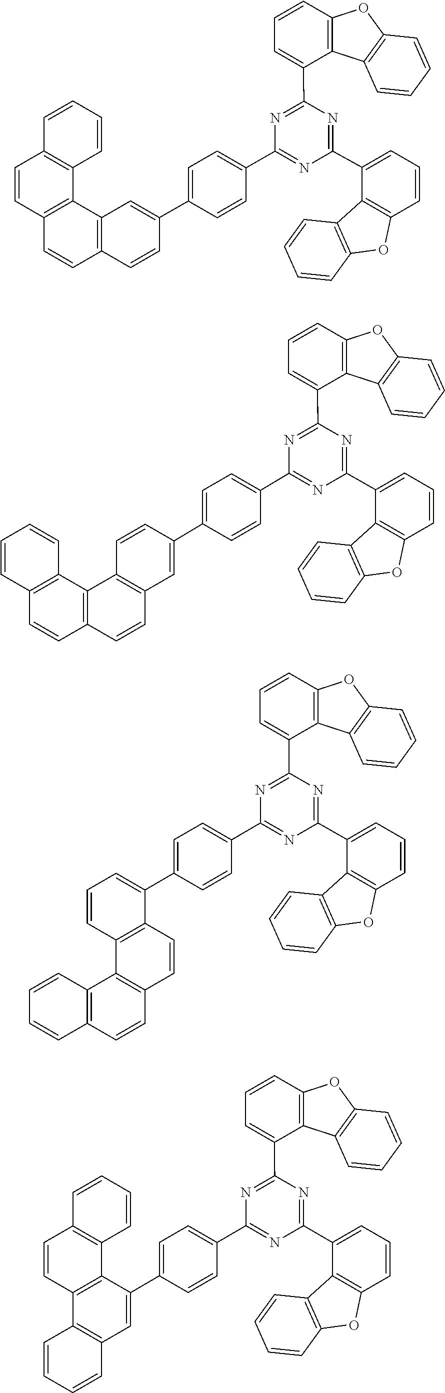

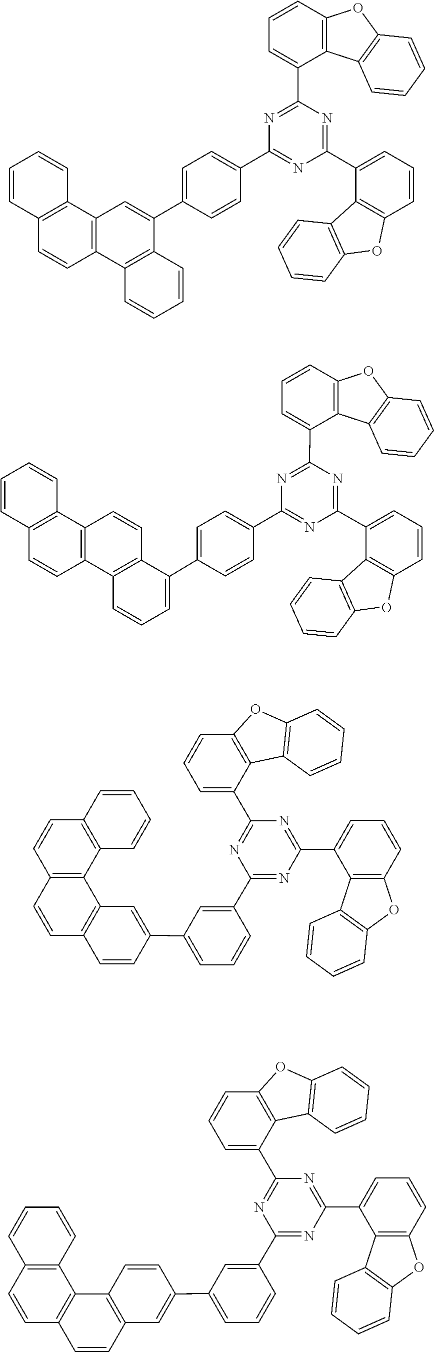 [SMILES: c1ccc2c(c1)ccc1c3ccccc3c(-c3ccc(-c4nc(-c5cccc6oc7ccccc7c56)nc(-c5cccc6oc7ccccc7c56)n4)cc3)cc21 ; c1ccc2c(c1)ccc1c3cccc(-c4ccc(-c5nc(-c6cccc7oc8ccccc8c67)nc(-c6cccc7oc8ccccc8c67)n5)cc4)c3ccc21 ; c1cc(-c2ccc3ccc4ccc5ccccc5c4c3c2)cc(-c2nc(-c3cccc4oc5ccccc5c34)nc(-c3cccc4oc5ccccc5c34)n2)c1 ; c1cc(-c2ccc3c(ccc4ccc5ccccc5c43)c2)cc(-c2nc(-c3cccc4oc5ccccc5c34)nc(-c3cccc4oc5ccccc5c34)n2)c1]

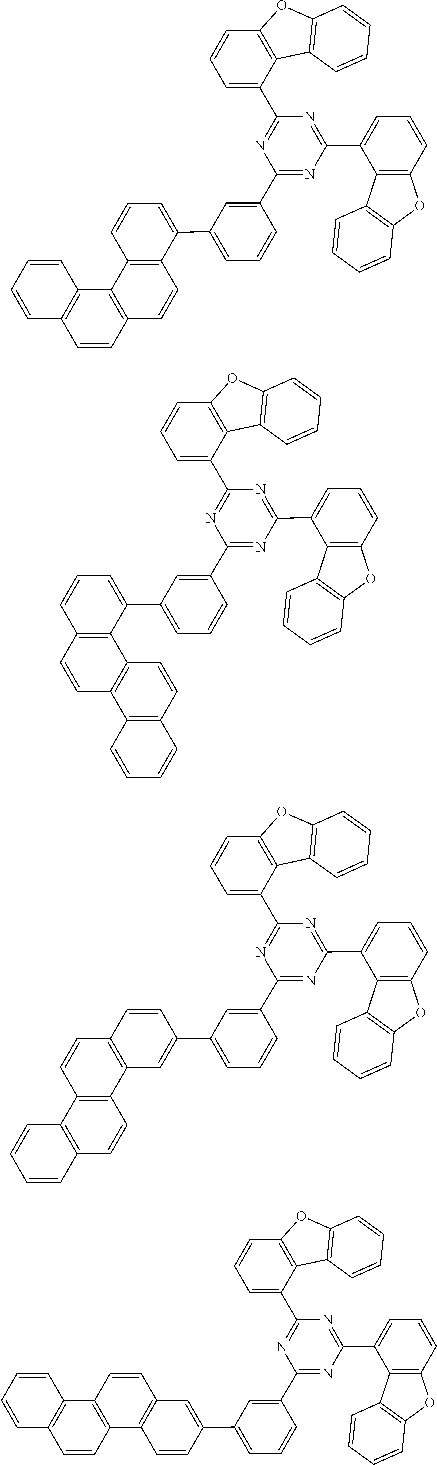 [SMILES: c1cc(-c2nc(-c3cccc4oc5ccccc5c34)nc(-c3cccc4oc5ccccc5c34)n2)cc(-c2cccc3c2ccc2ccc4ccccc4c23)c1 ; c1cc(-c2nc(-c3cccc4oc5ccccc5c34)nc(-c3cccc4oc5ccccc5c34)n2)cc(-c2cccc3ccc4c5ccccc5ccc4c23)c1 ; c1cc(-c2ccc3ccc4c5ccccc5ccc4c3c2)cc(-c2nc(-c3cccc4oc5ccccc5c34)nc(-c3cccc4oc5ccccc5c34)n2)c1 ; c1cc(-c2ccc3c(ccc4c5ccccc5ccc34)c2)cc(-c2nc(-c3cccc4oc5ccccc5c34)nc(-c3cccc4oc5ccccc5c34)n2)c1]

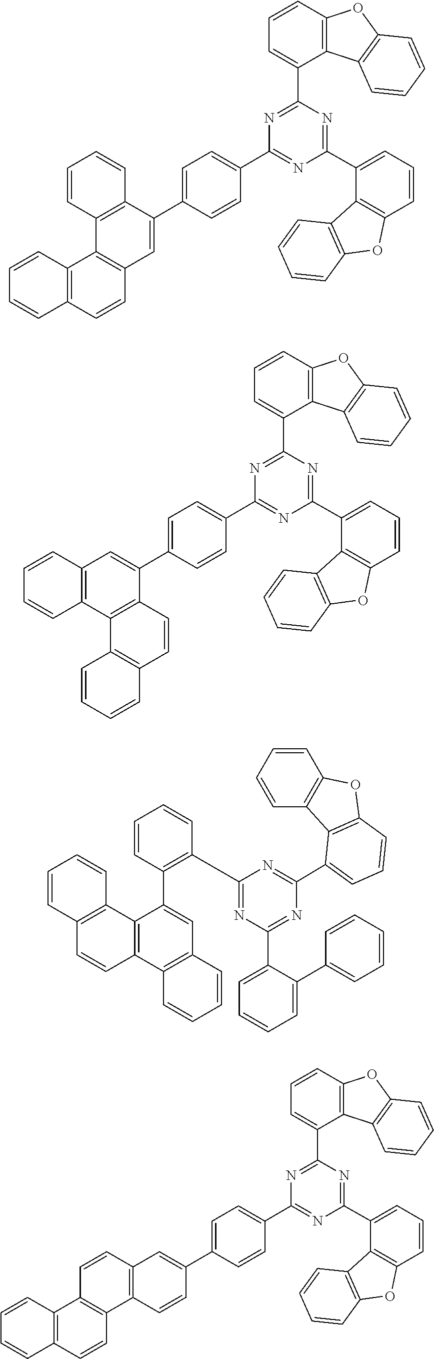 [SMILES: c1ccc2c(c1)ccc1cc(-c3ccc(-c4nc(-c5cccc6oc7ccccc7c56)nc(-c5cccc6oc7ccccc7c56)n4)cc3)c3ccccc3c12 ; c1ccc2c(c1)ccc1c(-c3ccc(-c4nc(-c5cccc6oc7ccccc7c56)nc(-c5cccc6oc7ccccc7c56)n4)cc3)cc3ccccc3c12 ; c1ccc(-c2ccccc2-c2nc(-c3ccccc3-c3cc4ccccc4c4ccc5ccccc5c34)nc(-c3cccc4oc5ccccc5c34)n2)cc1 ; c1ccc2c(c1)ccc1c3ccc(-c4ccc(-c5nc(-c6cccc7oc8ccccc8c67)nc(-c6cccc7oc8ccccc8c67)n5)cc4)cc3ccc21]

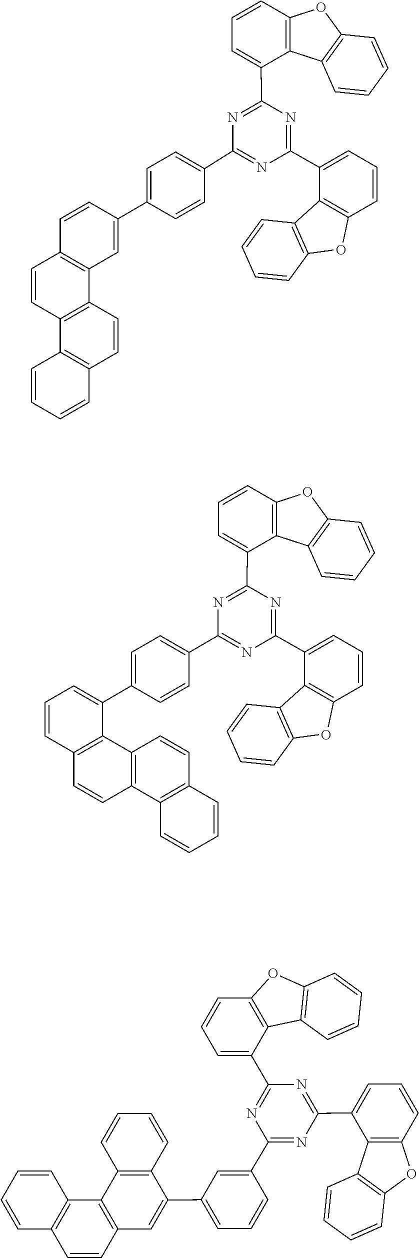 [SMILES: c1ccc2c(c1)ccc1c3cc(-c4ccc(-c5nc(-c6cccc7oc8ccccc8c67)nc(-c6cccc7oc8ccccc8c67)n5)cc4)ccc3ccc21 ; c1ccc2c(c1)ccc1c2ccc2cccc(-c3ccc(-c4nc(-c5cccc6oc7ccccc7c56)nc(-c5cccc6oc7ccccc7c56)n4)cc3)c21 ; c1cc(-c2nc(-c3cccc4oc5ccccc5c34)nc(-c3cccc4oc5ccccc5c34)n2)cc(-c2cc3ccc4ccccc4c3c3ccccc23)c1]

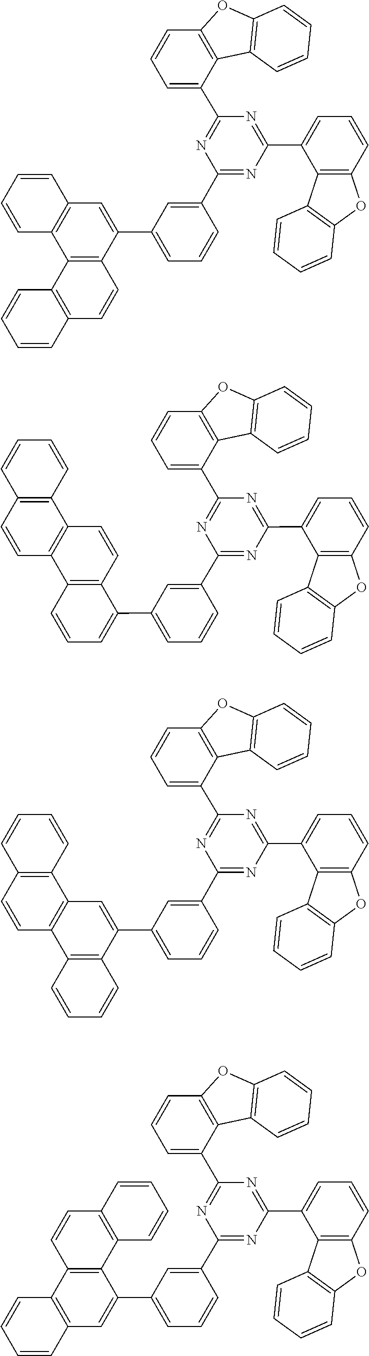 [SMILES: c1cc(-c2nc(-c3cccc4oc5ccccc5c34)nc(-c3cccc4oc5ccccc5c34)n2)cc(-c2cc3ccccc3c3c2ccc2ccccc23)c1 ; c1cc(-c2nc(-c3cccc4oc5ccccc5c34)nc(-c3cccc4oc5ccccc5c34)n2)cc(-c2cccc3c2ccc2c4ccccc4ccc32)c1 ; c1cc(-c2nc(-c3cccc4oc5ccccc5c34)nc(-c3cccc4oc5ccccc5c34)n2)cc(-c2cc3c4ccccc4ccc3c3ccccc23)c1 ; c1cc(-c2nc(-c3cccc4oc5ccccc5c34)nc(-c3cccc4oc5ccccc5c34)n2)cc(-c2cc3ccccc3c3ccc4ccccc4c23)c1]

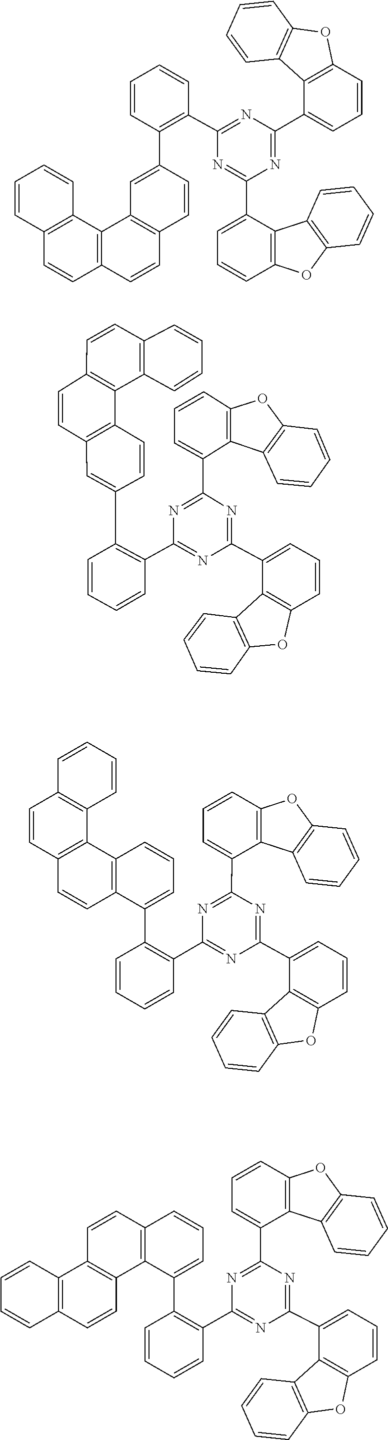

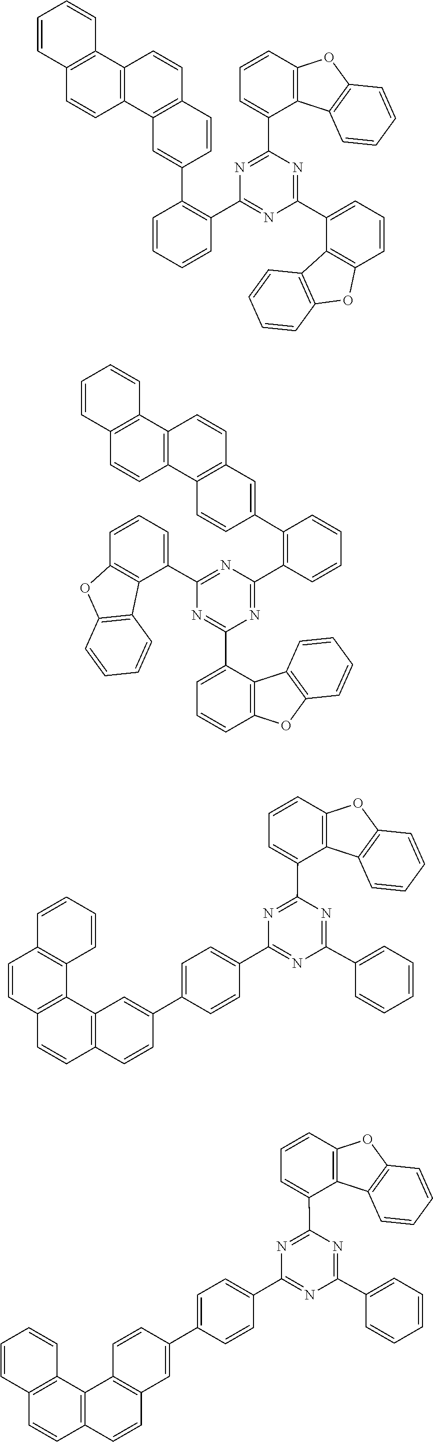 [SMILES: c1ccc(-c2nc(-c3cccc4oc5ccccc5c34)nc(-c3cccc4oc5ccccc5c34)n2)c(-c2ccc3ccc4c5ccccc5ccc4c3c2)c1 ; c1ccc(-c2nc(-c3cccc4oc5ccccc5c34)nc(-c3cccc4oc5ccccc5c34)n2)c(-c2ccc3c(ccc4c5ccccc5ccc34)c2)c1 ; c1ccc(-c2nc(-c3ccc(-c4ccc5ccc6ccc7ccccc7c6c5c4)cc3)nc(-c3cccc4oc5ccccc5c34)n2)cc1 ; c1ccc(-c2nc(-c3ccc(-c4ccc5c(ccc6ccc7ccccc7c65)c4)cc3)nc(-c3cccc4oc5ccccc5c34)n2)cc1]

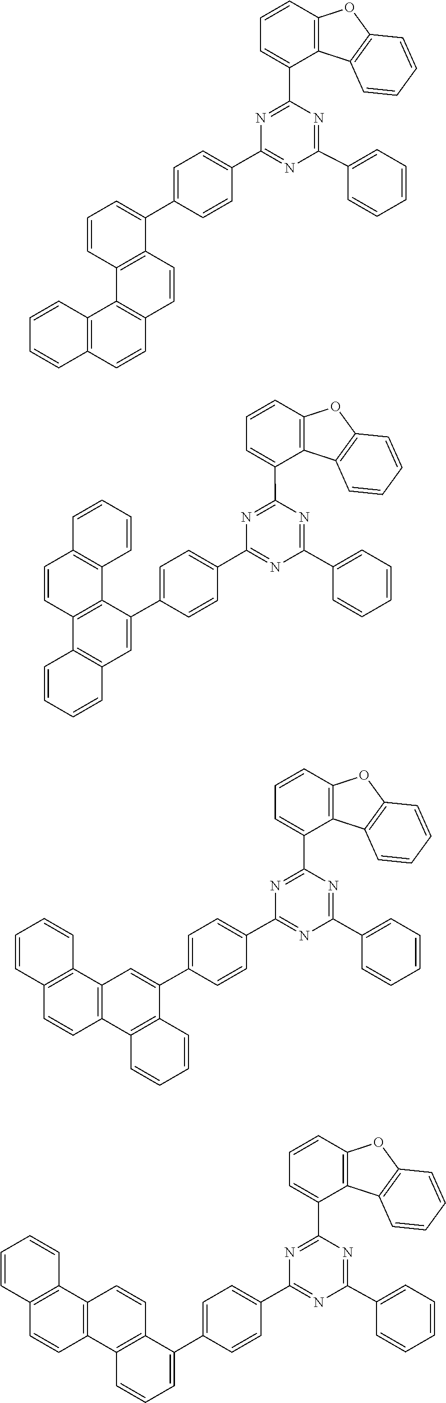 [SMILES: c1ccc(-c2nc(-c3ccc(-c4cccc5c4ccc4ccc6ccccc6c45)cc3)nc(-c3cccc4oc5ccccc5c34)n2)cc1 ; c1ccc(-c2nc(-c3ccc(-c4cc5ccccc5c5ccc6ccccc6c45)cc3)nc(-c3cccc4oc5ccccc5c34)n2)cc1 ; c1ccc(-c2nc(-c3ccc(-c4cc5c6ccccc6ccc5c5ccccc45)cc3)nc(-c3cccc4oc5ccccc5c34)n2)cc1 ; c1ccc(-c2nc(-c3ccc(-c4cccc5c4ccc4c6ccccc6ccc54)cc3)nc(-c3cccc4oc5ccccc5c34)n2)cc1]

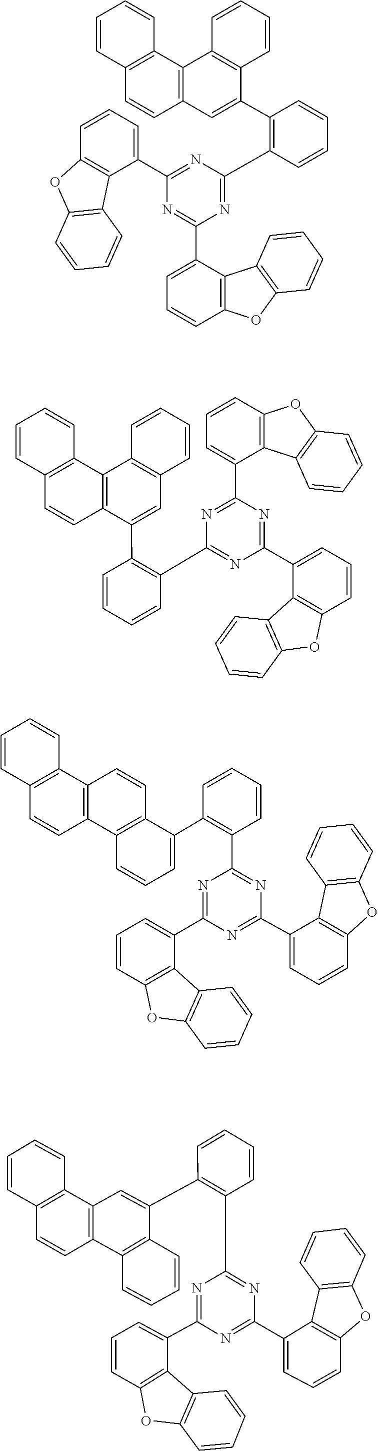 [SMILES: c1ccc(-c2cc3ccc4ccccc4c3c3ccccc23)c(-c2nc(-c3cccc4oc5ccccc5c34)nc(-c3cccc4oc5ccccc5c34)n2)c1 ; c1ccc(-c2cc3ccccc3c3c2ccc2ccccc23)c(-c2nc(-c3cccc4oc5ccccc5c34)nc(-c3cccc4oc5ccccc5c34)n2)c1 ; c1ccc(-c2cccc3c2ccc2c4ccccc4ccc32)c(-c2nc(-c3cccc4oc5ccccc5c34)nc(-c3cccc4oc5ccccc5c34)n2)c1 ; c1ccc(-c2cc3c4ccccc4ccc3c3ccccc23)c(-c2nc(-c3cccc4oc5ccccc5c34)nc(-c3cccc4oc5ccccc5c34)n2)c1]

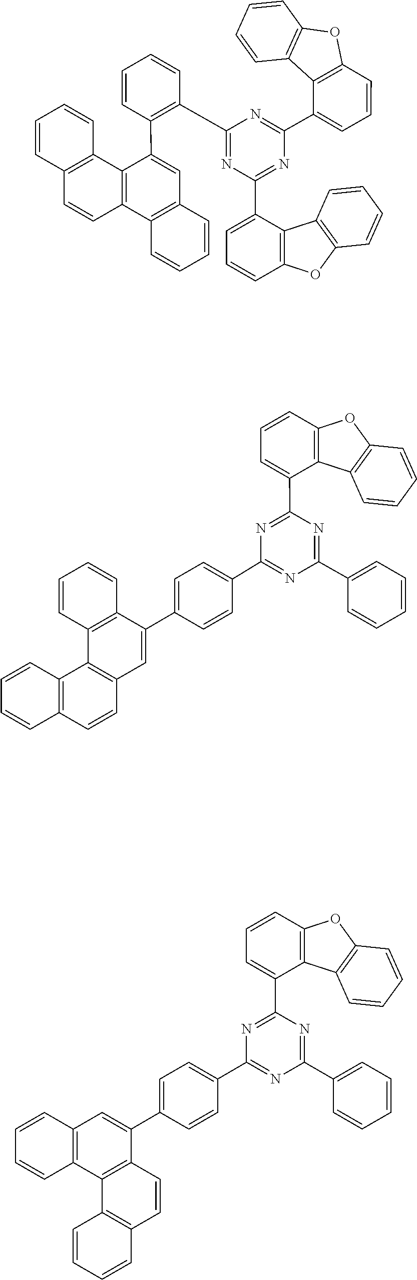 [SMILES: c1ccc(-c2cc3ccccc3c3ccc4ccccc4c23)c(-c2nc(-c3cccc4oc5ccccc5c34)nc(-c3cccc4oc5ccccc5c34)n2)c1 ; c1ccc(-c2nc(-c3ccc(-c4cc5ccc6ccccc6c5c5ccccc45)cc3)nc(-c3cccc4oc5ccccc5c34)n2)cc1 ; c1ccc(-c2nc(-c3ccc(-c4cc5ccccc5c5c4ccc4ccccc45)cc3)nc(-c3cccc4oc5ccccc5c34)n2)cc1]

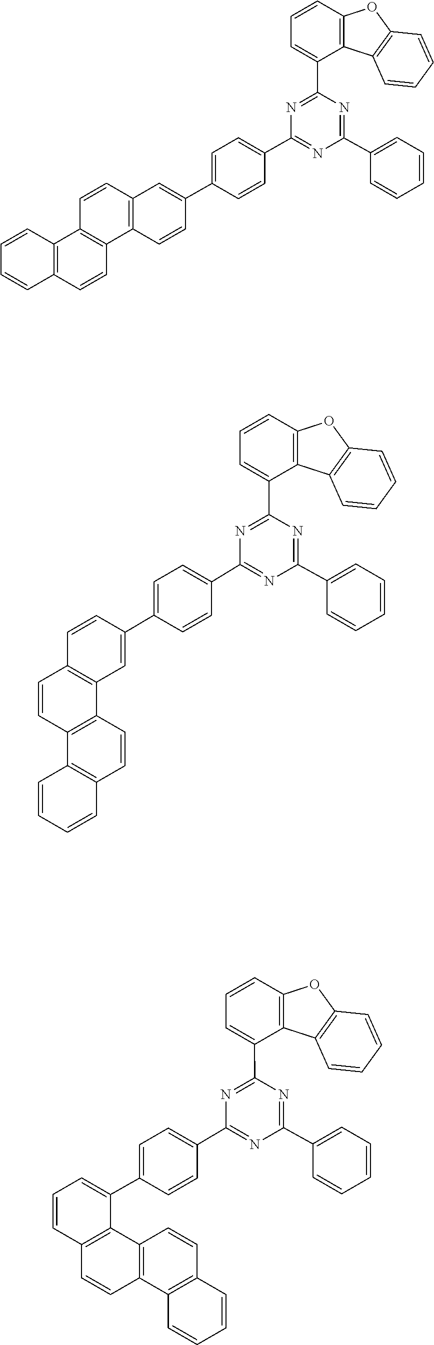 [SMILES: c1ccc(-c2nc(-c3ccc(-c4ccc5c(ccc6c7ccccc7ccc56)c4)cc3)nc(-c3cccc4oc5ccccc5c34)n2)cc1 ; c1ccc(-c2nc(-c3ccc(-c4ccc5ccc6c7ccccc7ccc6c5c4)cc3)nc(-c3cccc4oc5ccccc5c34)n2)cc1 ; c1ccc(-c2nc(-c3ccc(-c4cccc5ccc6c7ccccc7ccc6c45)cc3)nc(-c3cccc4oc5ccccc5c34)n2)cc1]

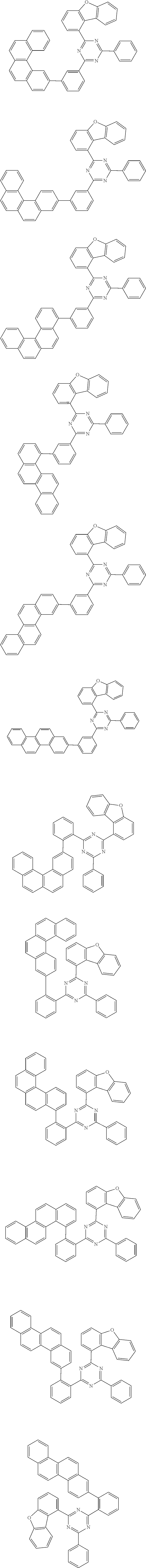

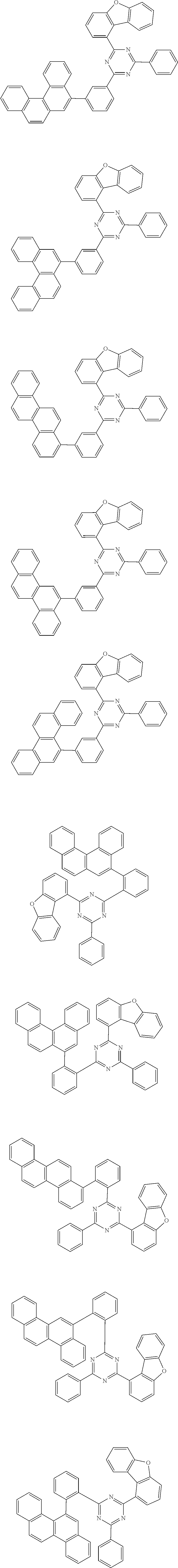

[chemical structure]

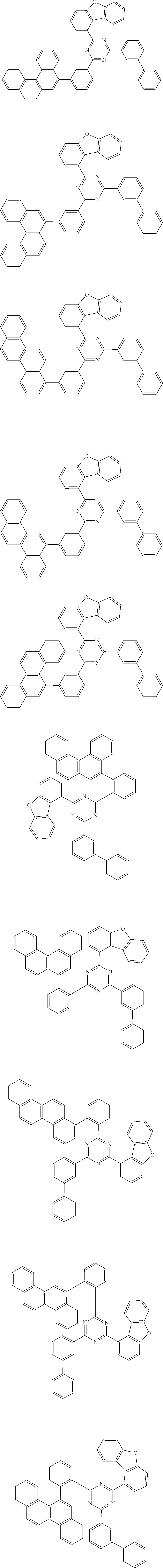

[chemical structure]

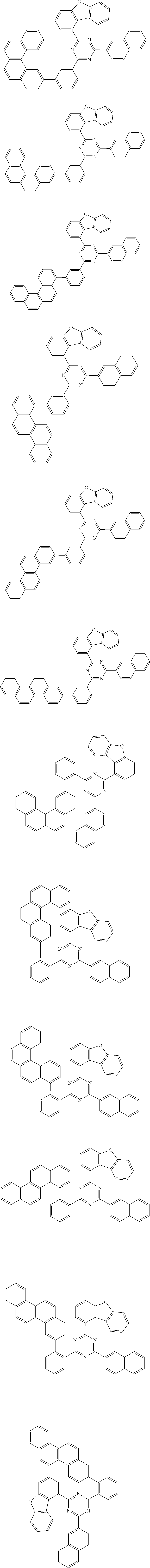

9. The organic light emitting device of claim 1, wherein Ar₄ is hydrogen, phenyl, naphthyl, or biphenyl.

10. The organic light emitting device of claim 1, wherein Ar₅ and Ar₆ are each independently phenyl, phenyl substituted with five deuteriums, phenyl substituted with naphthyl, biphenyl, biphenyl substituted with four deuteriums, biphenyl substituted with nine deuteriums, terphenyl, terphenyl substituted with four deuteriums, quaterphenyl, naphthyl, naphthyl substituted with phenyl, phenanthrenyl, triphenylene, dimethylfluorenyl, diphenylfluorenyl, carbazolyl, phenylcarbazolyl, dibenzofuranyl, dibenzothiophenyl, or dibenzofuranyl substituted with phenyl.

11. The organic light emitting device of claim 1, wherein Ar₅ and Ar₆ are each independently any one group selected from the group consisting of the following groups:

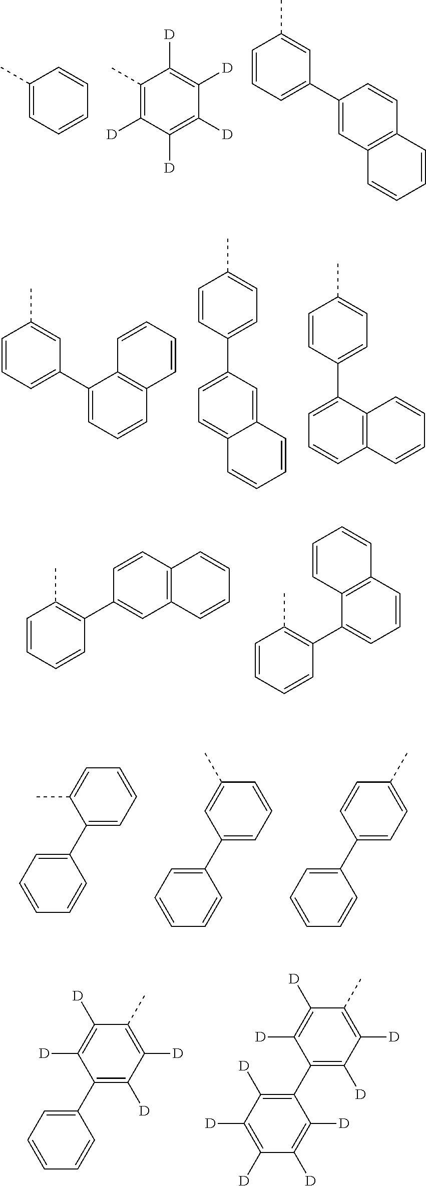

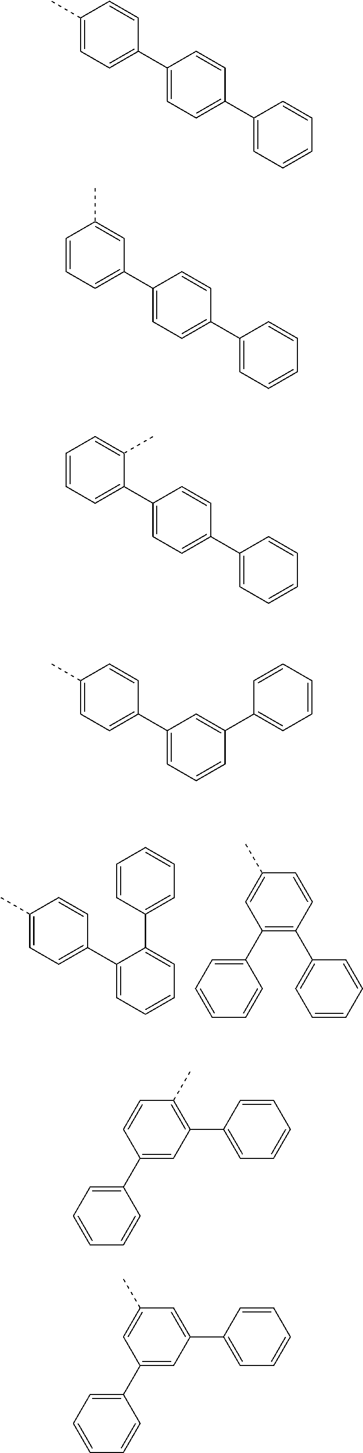

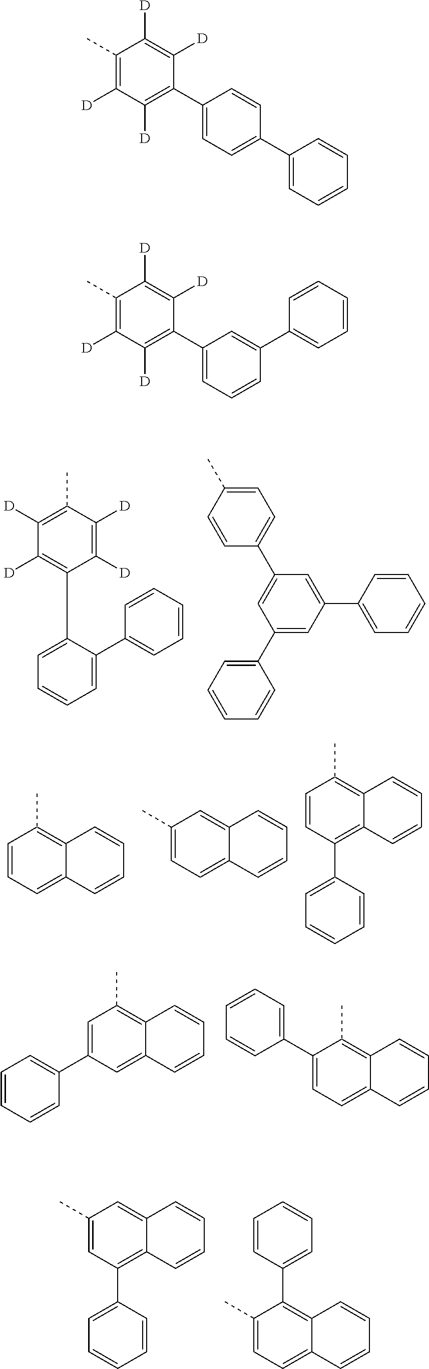

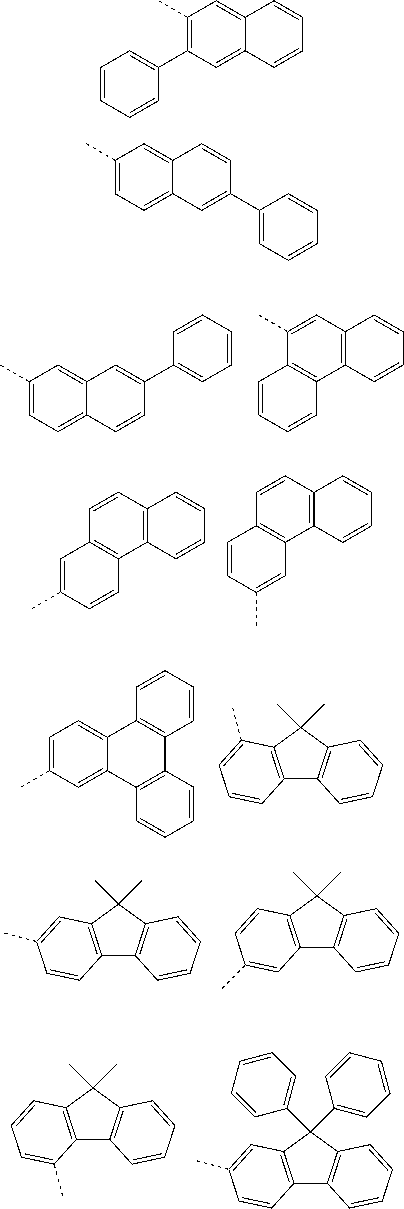

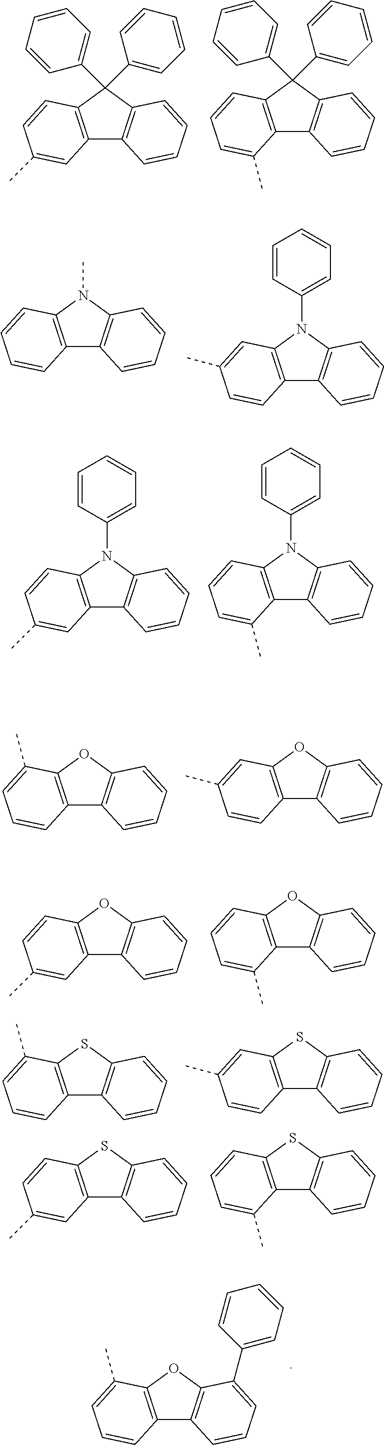

12. The organic light emitting device of claim 1, wherein La to Le are each independently a single bond, phenylene, phenylene substituted with four deuteriums, biphenylene, terphenylene, naphthylene, naphthylene substituted with phenyl, carbazolylene, carbazolylene substituted with phenyl, carbazolylene substituted with phenyl substituted with four deuteriums, dibenzofuranylene, dibenzofuranylene substituted with phenyl, dibenzofuranylene substituted with phenyl substituted with four deuteriums, or dimethylfluorenylene.

13. The organic light emitting device of claim 1, wherein L₄ to L₆ are each independently a single bond or any one group selected from the group consisting of the following groups:

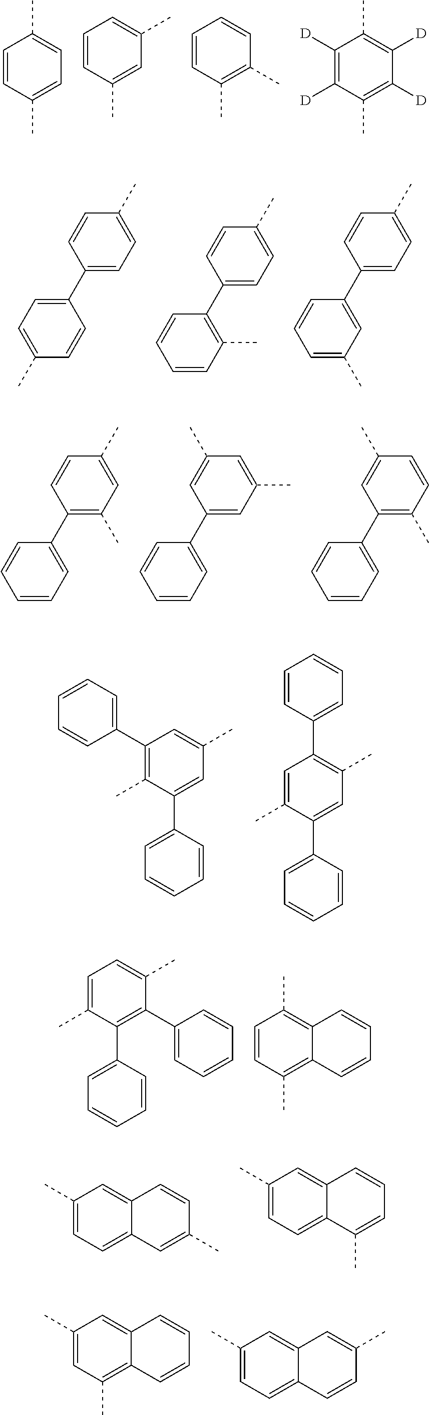

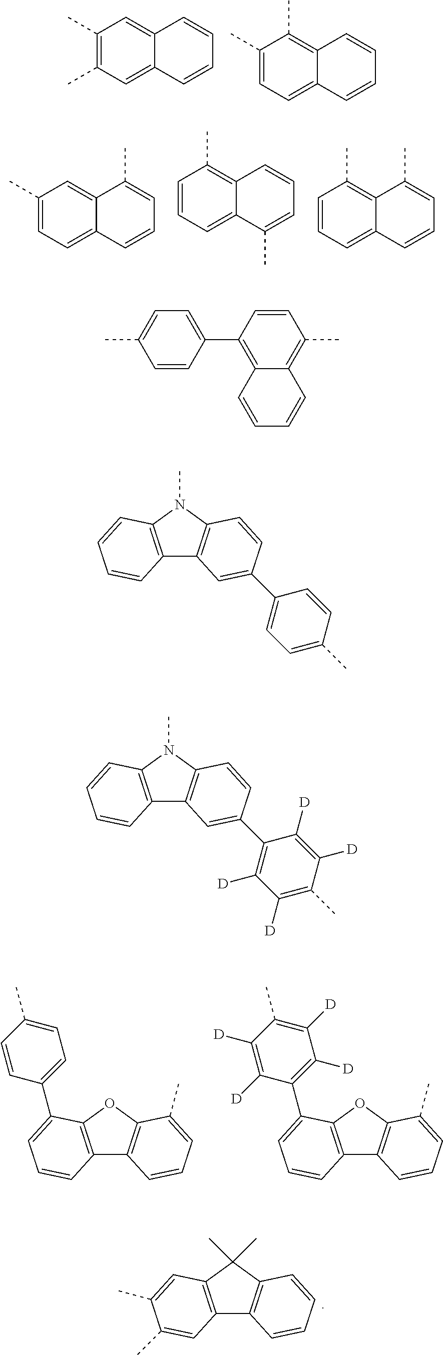

14. The organic light emitting device of claim 1, wherein L₇ is a substituted or unsubstituted phenylene, a substituted or unsubstituted biphenylene, or a substituted or unsubstituted naphthylene.

15. The organic light emitting device of claim 1, wherein the compound of Chemical Formula 2 is represented by the following Chemical Formula 2-1 or Chemical Formula 2-2: 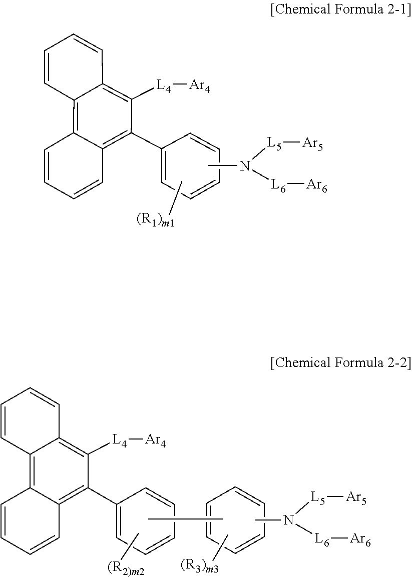
wherein in Chemical Formulae 2-1 and 2-2:
Ar₄ to Ar₆ and L₄ to L₆ are as defined in claim 1;
R₁ to R₃ are each independently hydrogen, deuterium, or substituted or unsubstituted C₆₋₆₀ aryl; and
m1 to m3 are each independently an integer of 0 to 4.

16. The organic light emitting device of claim 15, wherein R₁ to R₃ are each independently hydrogen or deuterium.

17. The organic light emitting device of claim 1, wherein the compound of Chemical Formula 2 is any one compound selected from the group consisting of the following compounds: 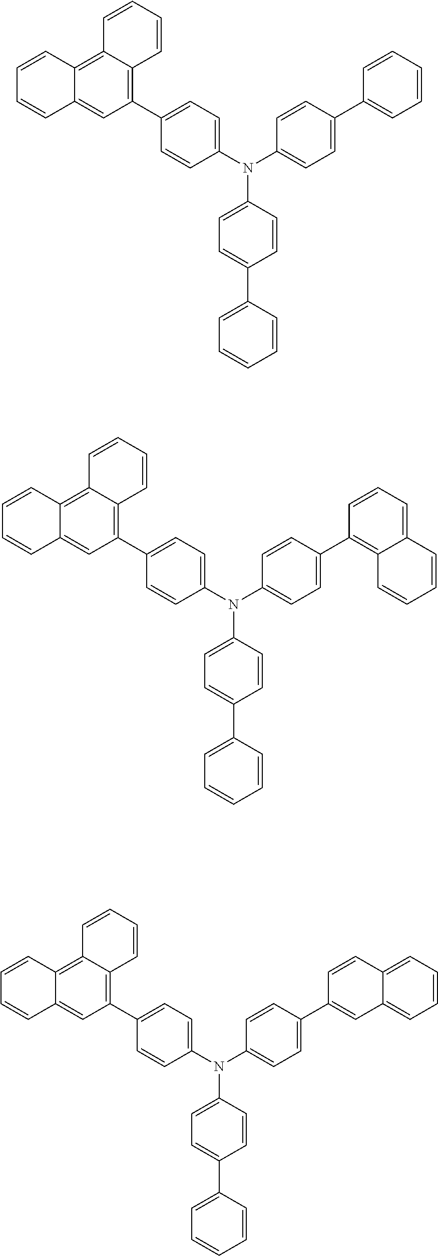 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1] 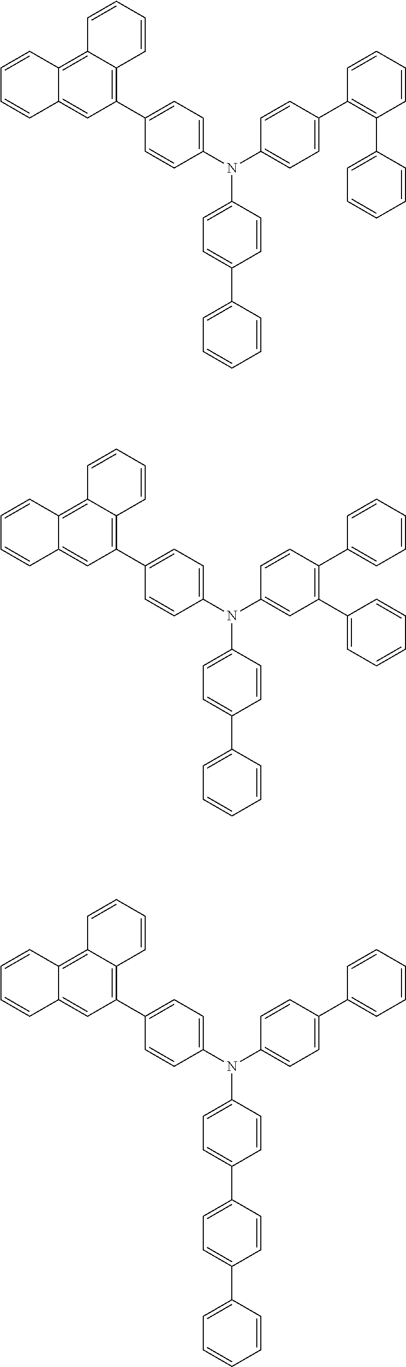 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c(-c4ccccc4)c3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1] 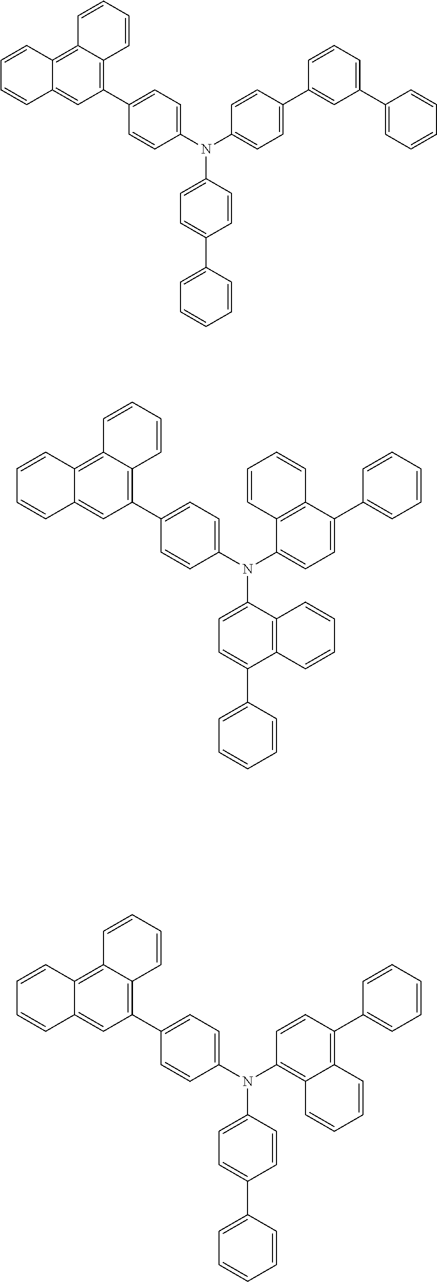 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1] 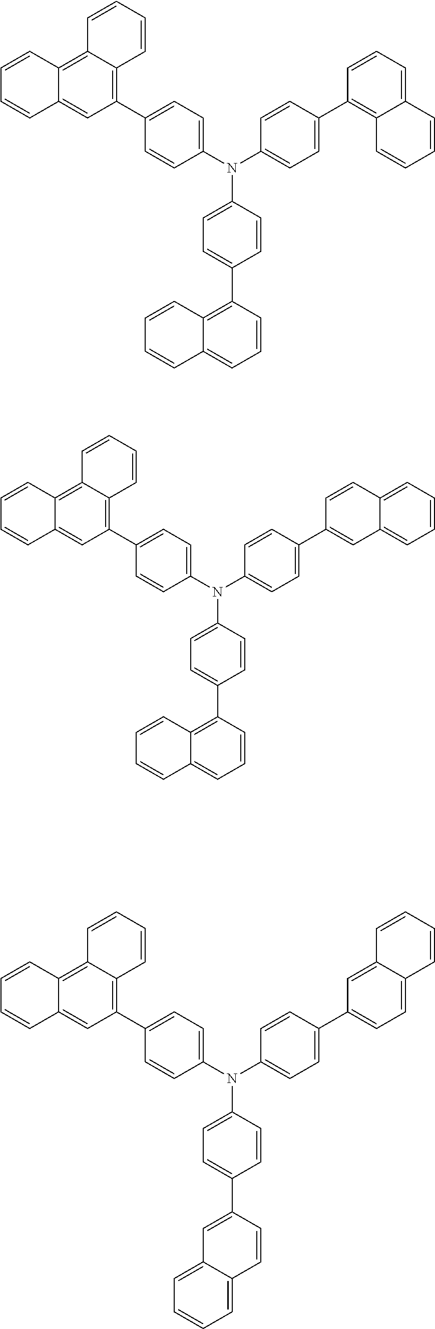 [SMILES: c1ccc2c(-c3ccc(N(c4ccc(-c5cccc6ccccc56)cc4)c4ccc(-c5cc6ccccc6c6ccccc56)cc4)cc3)cccc2c1 ; c1ccc2cc(-c3ccc(N(c4ccc(-c5cccc6ccccc56)cc4)c4ccc(-c5cc6ccccc6c6ccccc56)cc4)cc3)ccc2c1 ; c1ccc2cc(-c3ccc(N(c4ccc(-c5ccc6ccccc6c5)cc4)c4ccc(-c5cc6ccccc6c6ccccc56)cc4)cc3)ccc2c1] 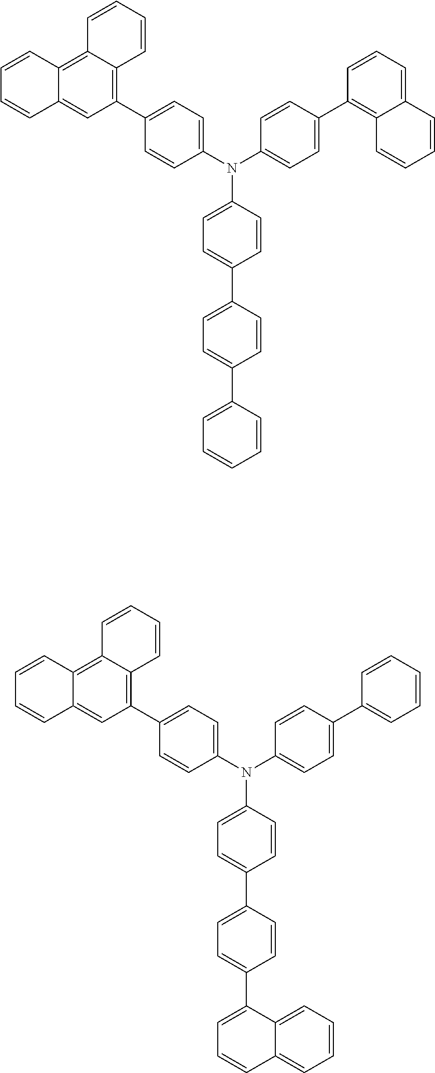 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1] 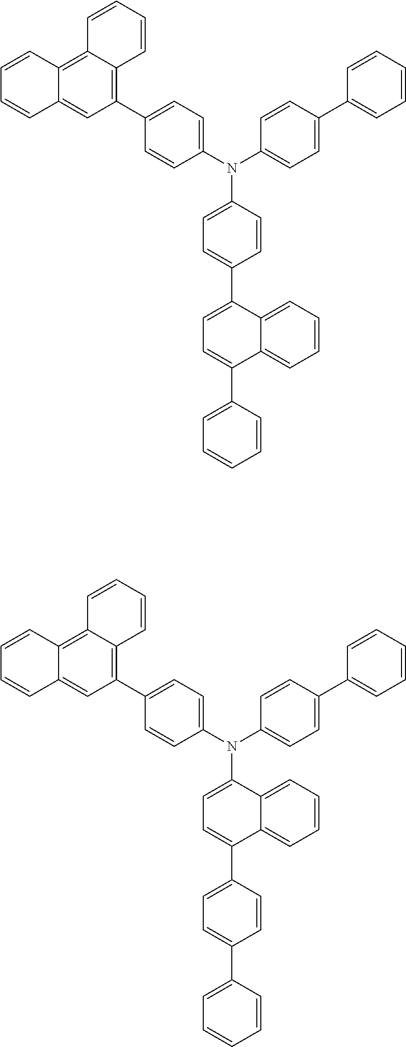 [SMILES: c1ccc(-c2ccc(N(c3ccc(-c4ccc(-c5ccccc5)c5ccccc45)cc3)c3ccc(-c4cc5ccccc5c5ccccc45)cc3)cc2)cc1 ; c1ccc(-c2ccc(-c3ccc(N(c4ccc(-c5ccccc5)cc4)c4ccc(-c5cc6ccccc6c6ccccc56)cc4)c4ccccc34)cc2)cc1] 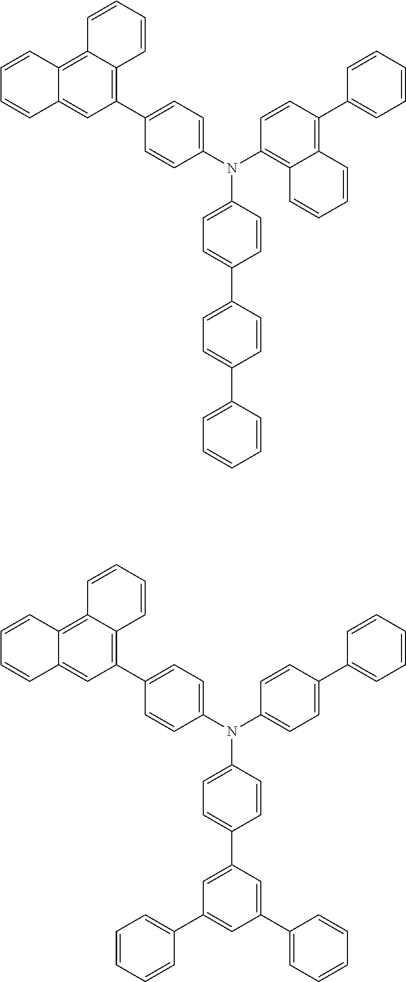 [SMILES: c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1] 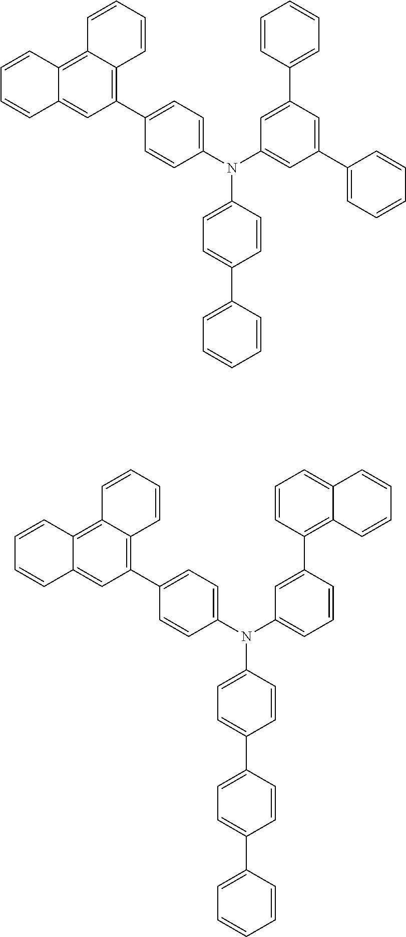 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(-c3ccc(N(c4ccc(-c5cc6ccccc6c6ccccc56)cc4)c4cccc(-c5cccc6ccccc56)c4)cc3)cc2)cc1] 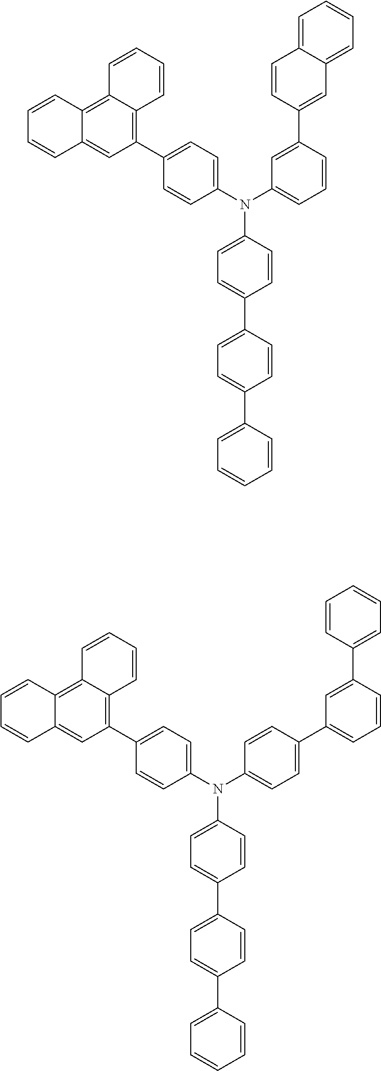 [SMILES: c1ccc(-c2ccc(-c3ccc(N(c4ccc(-c5cc6ccccc6c6ccccc56)cc4)c4cccc(-c5ccc6ccccc6c5)c4)cc3)cc2)cc1 ; c1ccc(-c2ccc(-c3ccc(N(c4ccc(-c5cccc(-c6ccccc6)c5)cc4)c4ccc(-c5cc6ccccc6c6ccccc56)cc4)cc3)cc2)cc1] 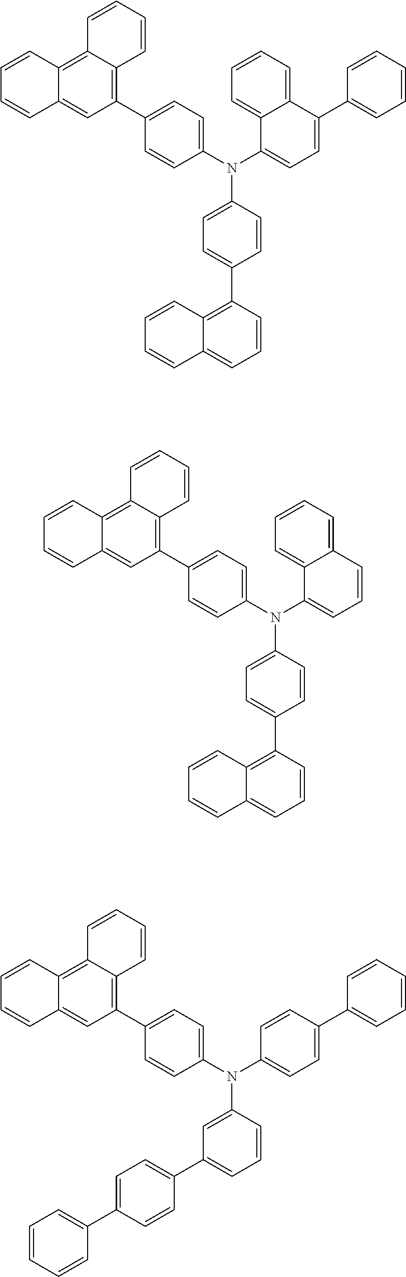 [SMILES: c1ccc(-c2ccc(N(c3ccc(-c4cccc5ccccc45)cc3)c3ccc(-c4cc5ccccc5c5ccccc45)cc3)c3ccccc23)cc1 ; c1ccc2c(-c3ccc(N(c4ccc(-c5cc6ccccc6c6ccccc56)cc4)c4cccc5ccccc45)cc3)cccc2c1 ; c1ccc(-c2ccc(-c3cccc(N(c4ccc(-c5ccccc5)cc4)c4ccc(-c5cc6ccccc6c6ccccc56)cc4)c3)cc2)cc1] 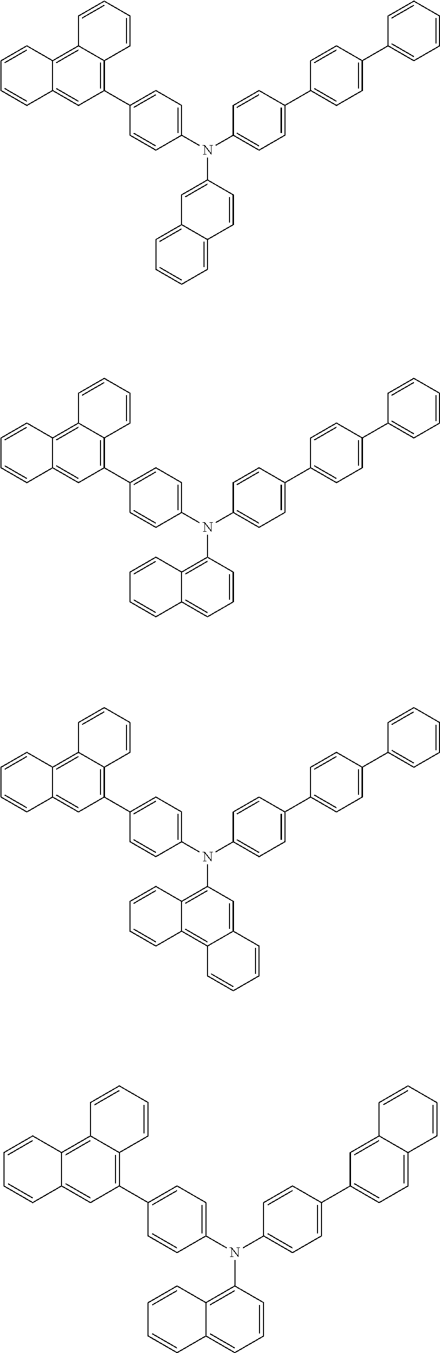 [SMILES: c1ccc(-c2ccc(-c3ccc(N(c4ccc(-c5cc6ccccc6c6ccccc56)cc4)c4ccc5ccccc5c4)cc3)cc2)cc1 ; c1ccc(-c2ccc(-c3ccc(N(c4ccc(-c5cc6ccccc6c6ccccc56)cc4)c4cccc5ccccc45)cc3)cc2)cc1 ; c1ccc(-c2ccc(-c3ccc(N(c4ccc(-c5cc6ccccc6c6ccccc56)cc4)c4cc5ccccc5c5ccccc45)cc3)cc2)cc1 ; c1ccc2cc(-c3ccc(N(c4ccc(-c5cc6ccccc6c6ccccc56)cc4)c4cccc5ccccc45)cc3)ccc2c1] 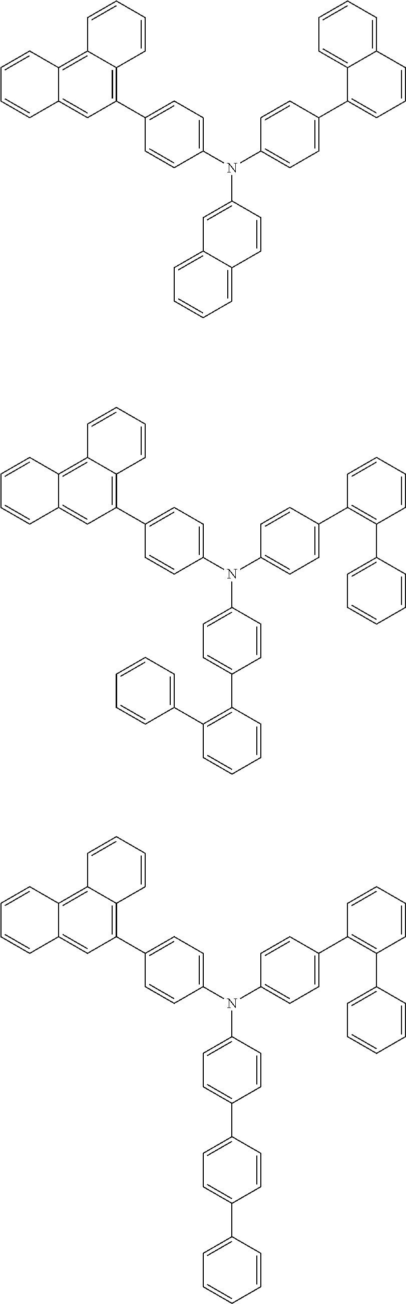 [SMILES: c1ccc2cc(N(c3ccc(-c4cccc5ccccc45)cc3)c3ccc(-c4cc5ccccc5c5ccccc45)cc3)ccc2c1 ; c1ccc(-c2ccccc2-c2ccc(N(c3ccc(-c4ccccc4-c4ccccc4)cc3)c3ccc(-c4cc5ccccc5c5ccccc45)cc3)cc2)cc1 ; c1ccc(-c2ccc(-c3ccc(N(c4ccc(-c5ccccc5-c5ccccc5)cc4)c4ccc(-c5cc6ccccc6c6ccccc56)cc4)cc3)cc2)cc1] 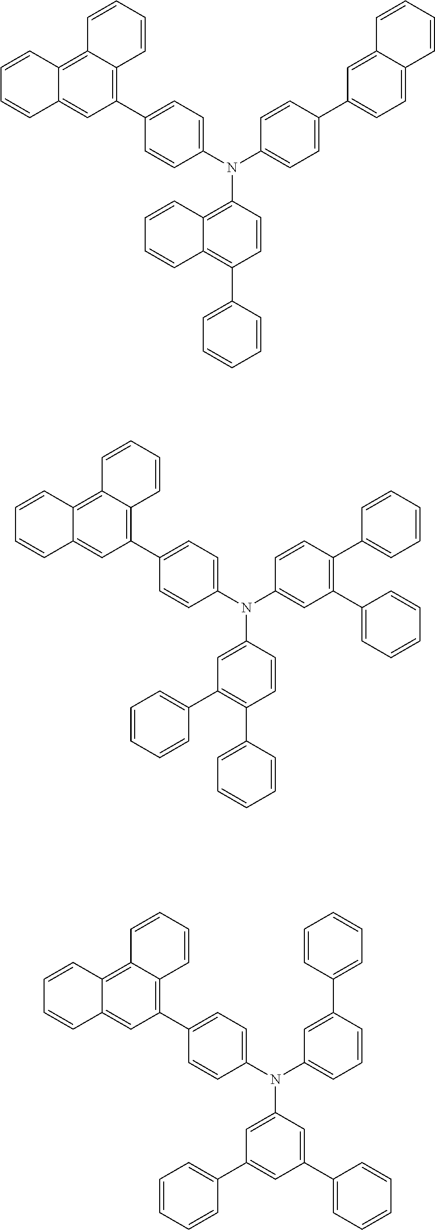 [SMILES: c1ccc(-c2ccc(N(c3ccc(-c4ccc5ccccc5c4)cc3)c3ccc(-c4cc5ccccc5c5ccccc45)cc3)c3ccccc23)cc1 ; c1ccc(-c2ccc(N(c3ccc(-c4cc5ccccc5c5ccccc45)cc3)c3ccc(-c4ccccc4)c(-c4ccccc4)c3)cc2-c2ccccc2)cc1 ; c1ccc(-c2cccc(N(c3ccc(-c4cc5ccccc5c5ccccc45)cc3)c3cc(-c4ccccc4)cc(-c4ccccc4)c3)c2)cc1] 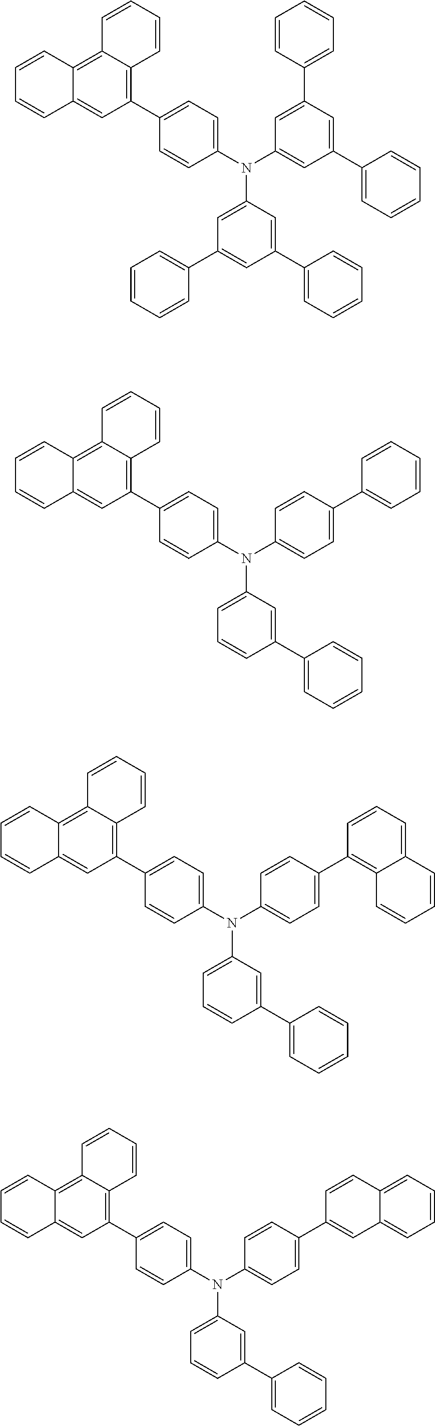 [SMILES: c1ccc(-c2cc(-c3ccccc3)cc(N(c3ccc(-c4cc5ccccc5c5ccccc45)cc3)c3cc(-c4ccccc4)cc(-c4ccccc4)c3)c2)cc1 ; c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1 ; c1ccc(-c2cccc(N(c3ccc(-c4ccc5ccccc5c4)cc3)c3ccc(-c4cc5ccccc5c5ccccc45)cc3)c2)cc1] 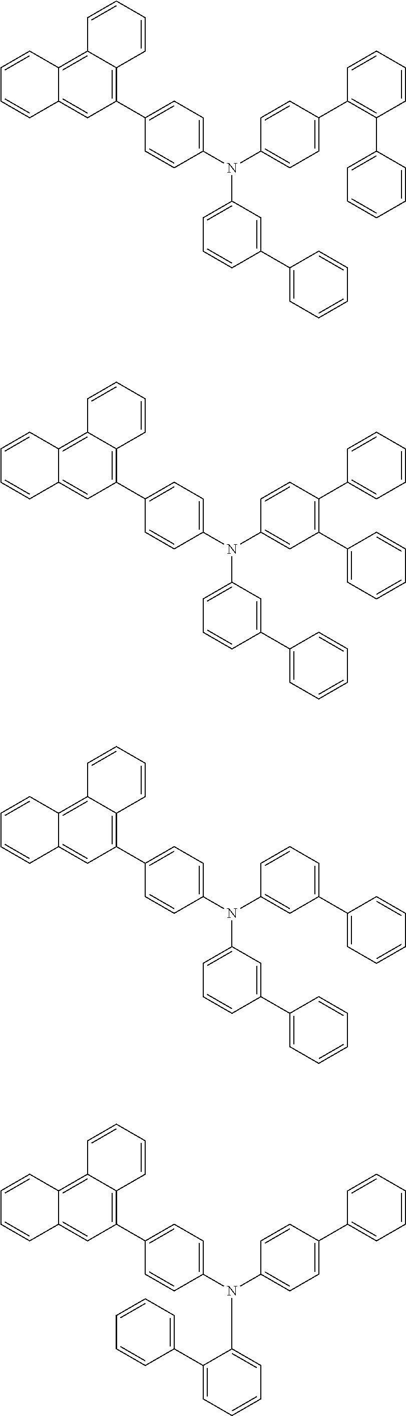 [SMILES: c1ccc(-c2cccc(N(c3ccc(-c4ccccc4-c4ccccc4)cc3)c3ccc(-c4cc5ccccc5c5ccccc45)cc3)c2)cc1 ; c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1 ; c1ccc(-c2ccc(N(c3ccc(-c4cc5ccccc5c5ccccc45)cc3)c3ccccc3-c3ccccc3)cc2)cc1] 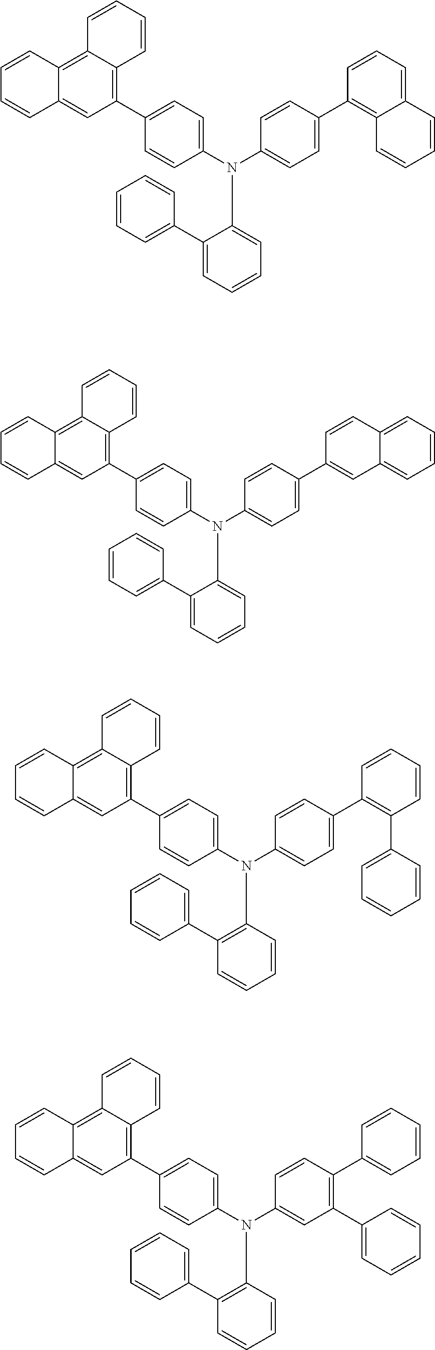 [SMILES: c1ccc(-c2ccccc2N(c2ccc(-c3cccc4ccccc34)cc2)c2ccc(-c3cc4ccccc4c4ccccc34)cc2)cc1 ; c1ccc(-c2ccccc2N(c2ccc(-c3ccc4ccccc4c3)cc2)c2ccc(-c3cc4ccccc4c4ccccc34)cc2)cc1 ; c1ccc(-c2ccccc2-c2ccc(N(c3ccc(-c4cc5ccccc5c5ccccc45)cc3)c3ccccc3-c3ccccc3)cc2)cc1 ; c1ccc(-c2ccc(N(c3ccc(-c4cc5ccccc5c5ccccc45)cc3)c3ccccc3-c3ccccc3)cc2-c2ccccc2)cc1] 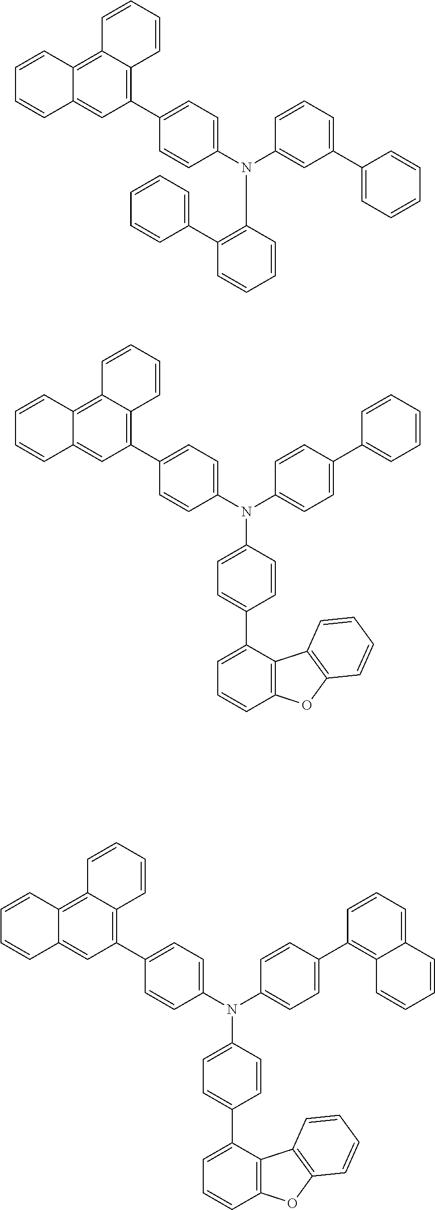 [SMILES: c1ccc(-c2cccc(N(c3ccc(-c4cc5ccccc5c5ccccc45)cc3)c3ccccc3-c3ccccc3)c2)cc1 ; c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1] 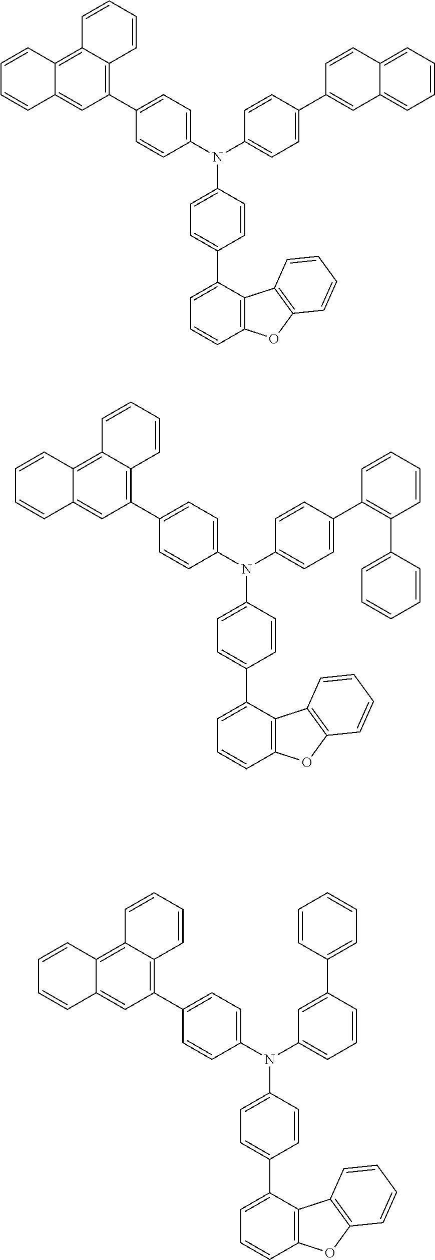 [SMILES: c1ccc2cc(-c3ccc(N(c4ccc(-c5cc6ccccc6c6ccccc56)cc4)c4ccc(-c5cccc6oc7ccccc7c56)cc4)cc3)ccc2c1 ; c1ccc(-c2ccccc2-c2ccc(N(c3ccc(-c4cc5ccccc5c5ccccc45)cc3)c3ccc(-c4cccc5oc6ccccc6c45)cc3)cc2)cc1 ; c1ccc(-c2cccc(N(c3ccc(-c4cc5ccccc5c5ccccc45)cc3)c3ccc(-c4cccc5oc6ccccc6c45)cc3)c2)cc1] 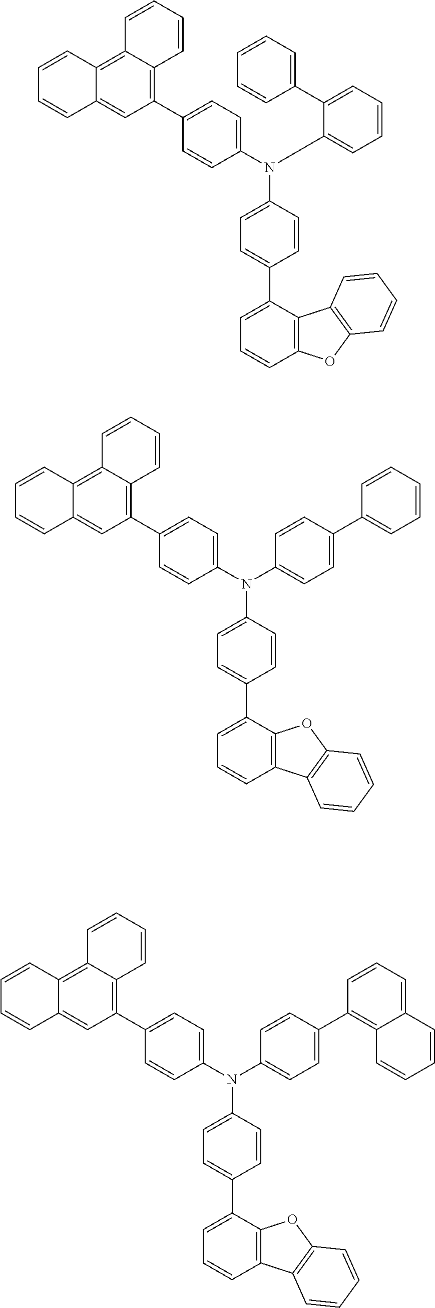 [SMILES: c1ccc(-c2ccccc2N(c2ccc(-c3cc4ccccc4c4ccccc34)cc2)c2ccc(-c3cccc4oc5ccccc5c34)cc2)cc1 ; c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1] 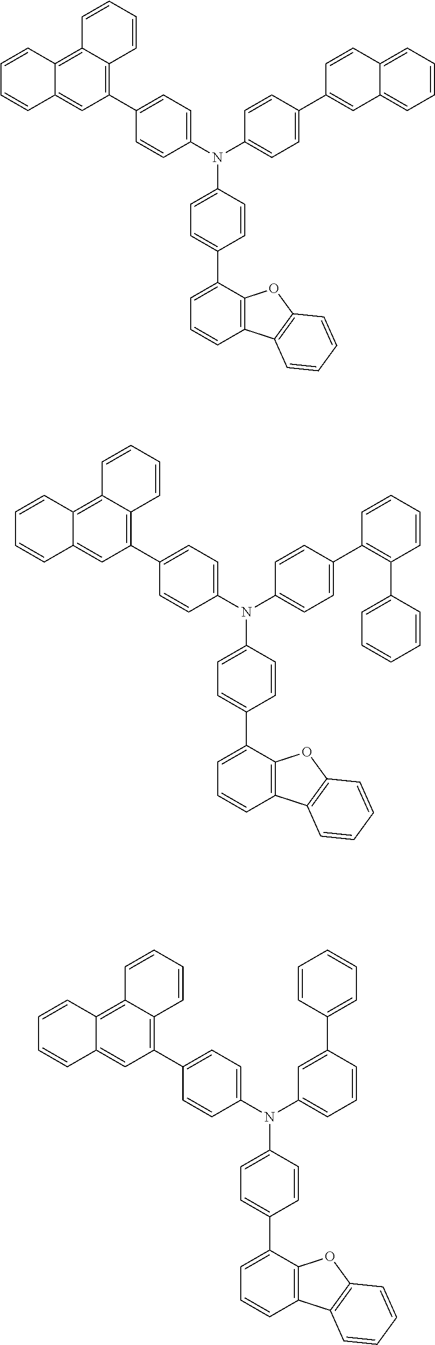 [SMILES: c1ccc2cc(-c3ccc(N(c4ccc(-c5cc6ccccc6c6ccccc56)cc4)c4ccc(-c5cccc6c5oc5ccccc56)cc4)cc3)ccc2c1 ; c1ccc(-c2ccccc2-c2ccc(N(c3ccc(-c4cc5ccccc5c5ccccc45)cc3)c3ccc(-c4cccc5c4oc4ccccc45)cc3)cc2)cc1 ; c1ccc(-c2cccc(N(c3ccc(-c4cc5ccccc5c5ccccc45)cc3)c3ccc(-c4cccc5c4oc4ccccc45)cc3)c2)cc1] 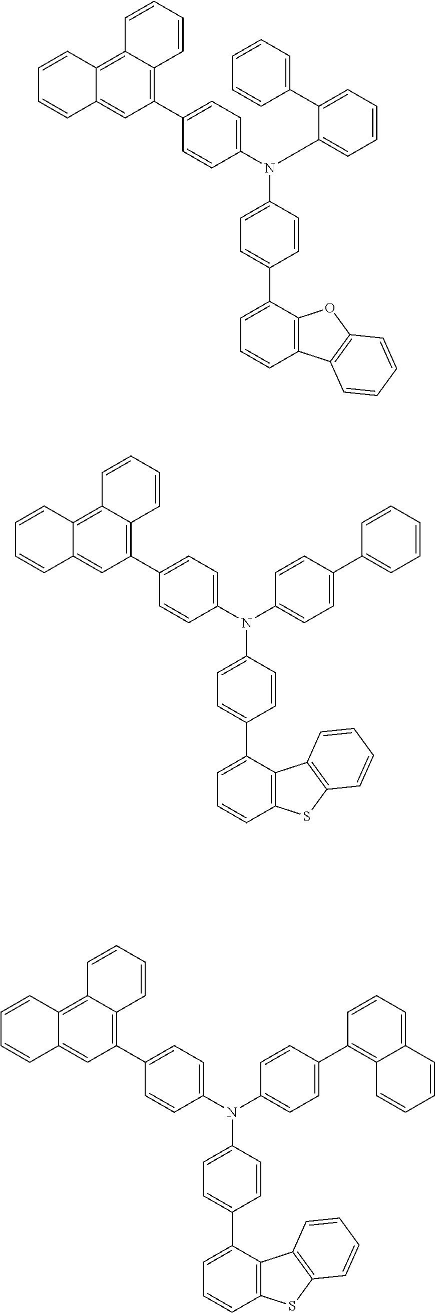 [SMILES: c1ccc(-c2ccccc2N(c2ccc(-c3cc4ccccc4c4ccccc34)cc2)c2ccc(-c3cccc4c3oc3ccccc34)cc2)cc1 ; c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1] 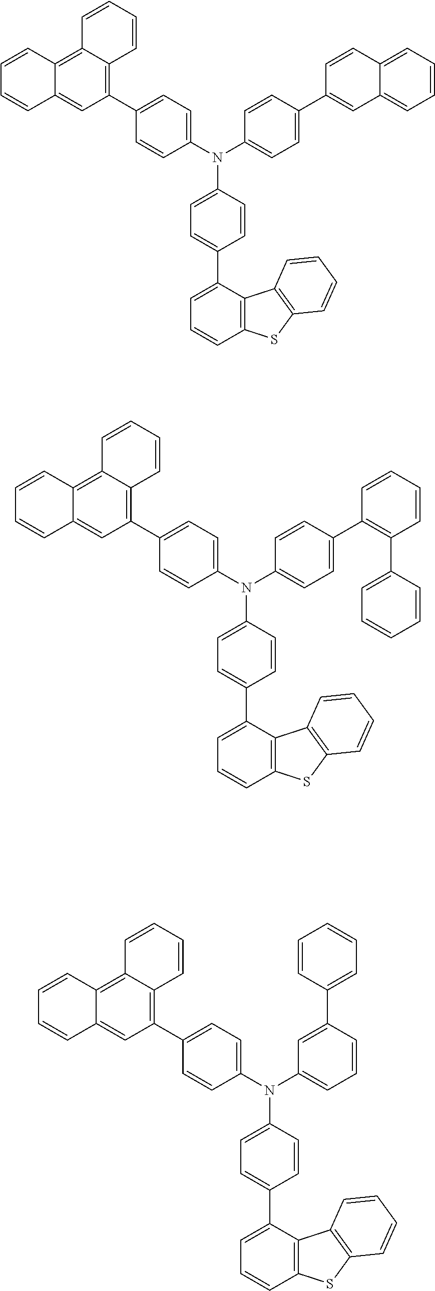 [SMILES: c1ccc2cc(-c3ccc(N(c4ccc(-c5cc6ccccc6c6ccccc56)cc4)c4ccc(-c5cccc6sc7ccccc7c56)cc4)cc3)ccc2c1 ; c1ccc(-c2ccccc2-c2ccc(N(c3ccc(-c4cc5ccccc5c5ccccc45)cc3)c3ccc(-c4cccc5sc6ccccc6c45)cc3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1] 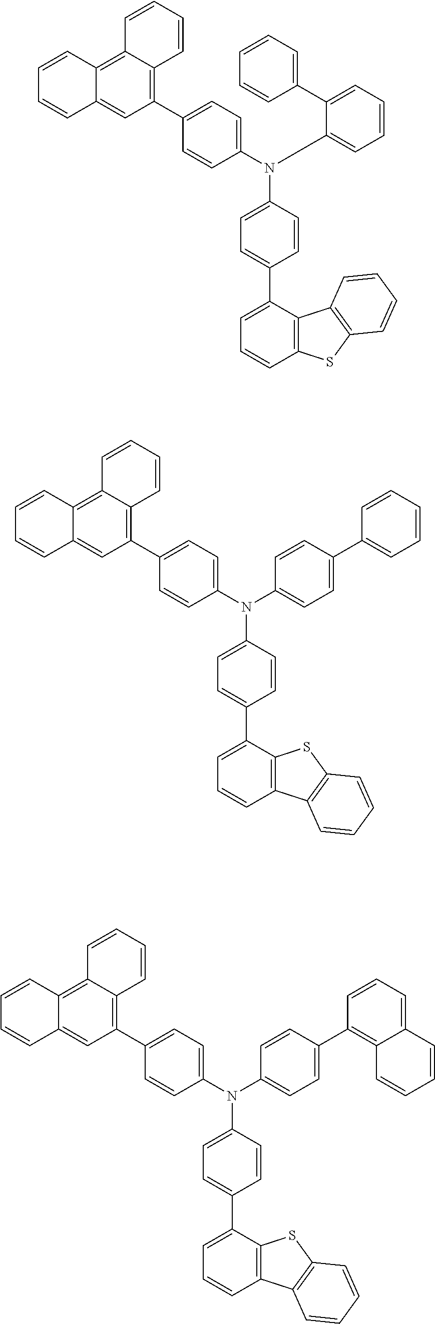 [SMILES: c1ccc(-c2ccccc2N(c2ccc(-c3cc4ccccc4c4ccccc34)cc2)c2ccc(-c3cccc4sc5ccccc5c34)cc2)cc1 ; c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1] 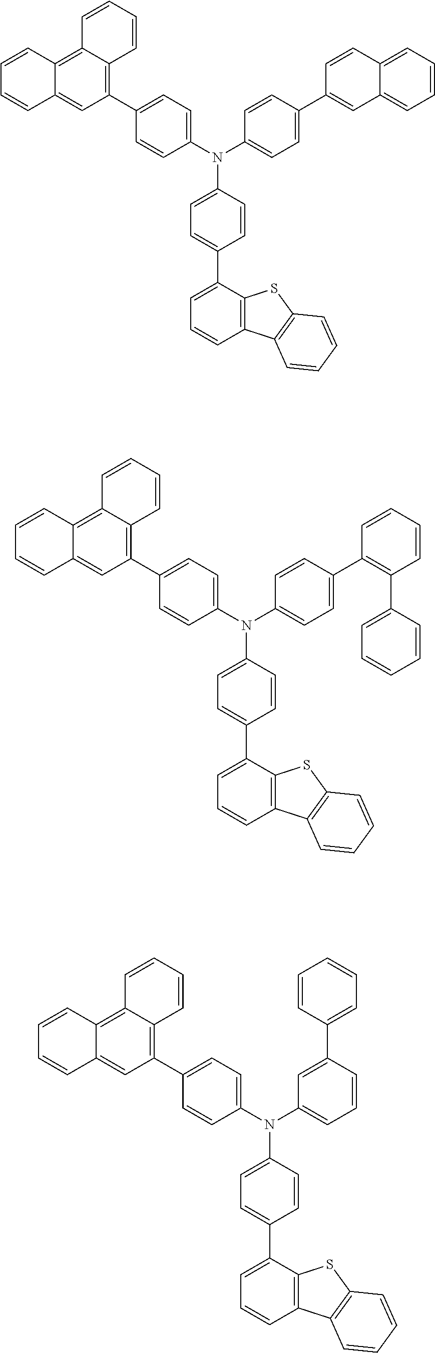 [SMILES: c1ccc2cc(-c3ccc(N(c4ccc(-c5cc6ccccc6c6ccccc56)cc4)c4ccc(-c5cccc6c5sc5ccccc56)cc4)cc3)ccc2c1 ; c1ccc(-c2ccccc2-c2ccc(N(c3ccc(-c4cc5ccccc5c5ccccc45)cc3)c3ccc(-c4cccc5c4sc4ccccc45)cc3)cc2)cc1 ; c1ccc(-c2cccc(N(c3ccc(-c4cc5ccccc5c5ccccc45)cc3)c3ccc(-c4cccc5c4sc4ccccc45)cc3)c2)cc1] 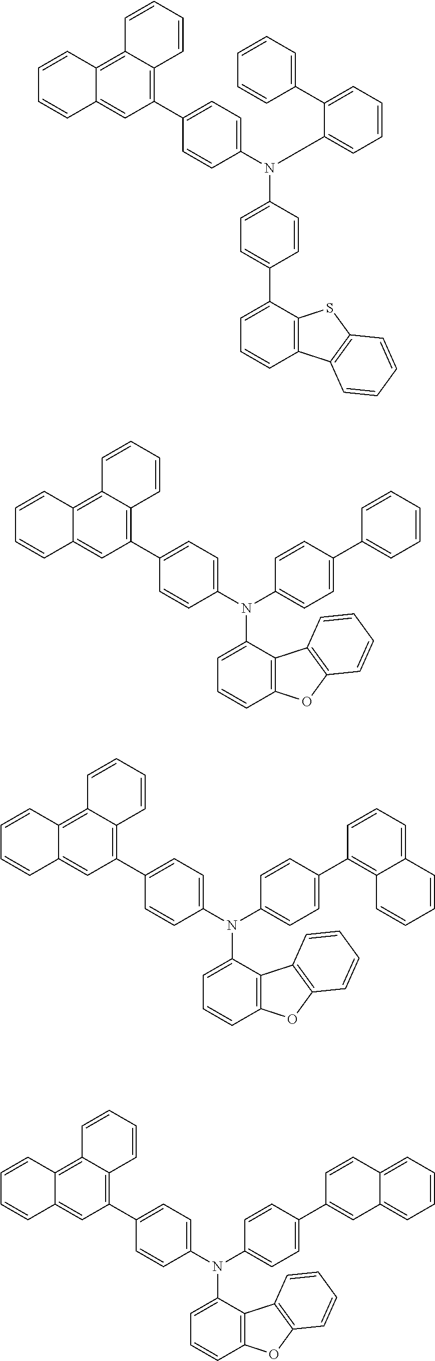 [SMILES: c1ccc(-c2ccccc2N(c2ccc(-c3cc4ccccc4c4ccccc34)cc2)c2ccc(-c3cccc4c3sc3ccccc34)cc2)cc1 ; c1ccc(-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)cc3)cccc2c1 ; c1ccc2cc(-c3ccc(N(c4ccc(-c5cc6ccccc6c6ccccc56)cc4)c4cccc5oc6ccccc6c45)cc3)ccc2c1] 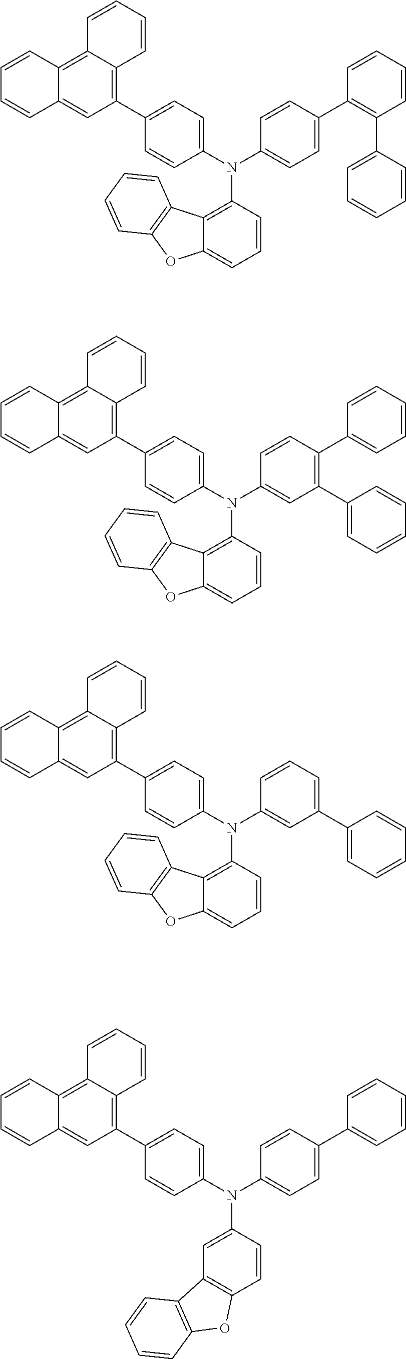 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2-c2ccccc2)cc1 ; c1ccc(-c2cccc(N(c3ccc(-c4cc5ccccc5c5ccccc45)cc3)c3cccc4oc5ccccc5c34)c2)cc1 ; c1ccc(-c2ccc(N(c3ccc(-c4cc5ccccc5c5ccccc45)cc3)c3ccc4oc5ccccc5c4c3)cc2)cc1] 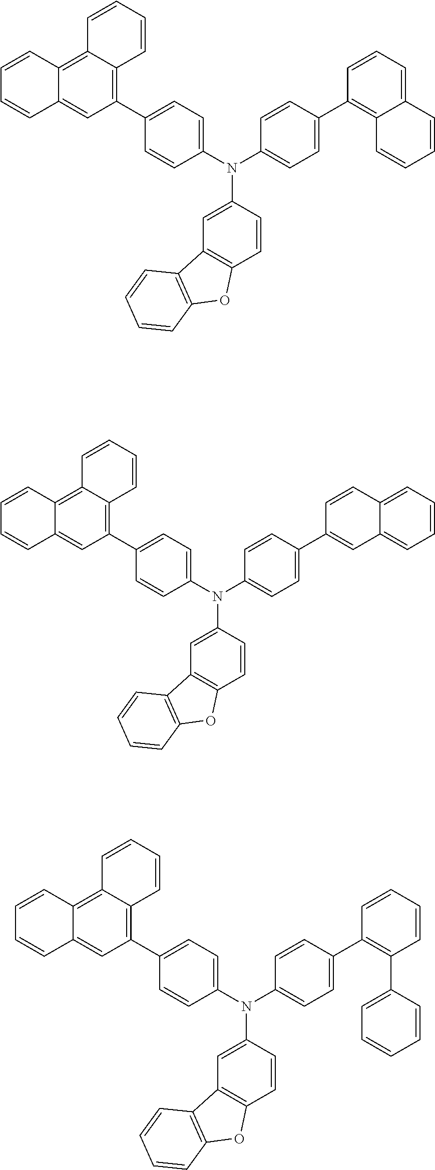 [SMILES: c1ccc2c(-c3ccc(N(c4ccc(-c5cc6ccccc6c6ccccc56)cc4)c4ccc5oc6ccccc6c5c4)cc3)cccc2c1 ; c1ccc2cc(-c3ccc(N(c4ccc(-c5cc6ccccc6c6ccccc56)cc4)c4ccc5oc6ccccc6c5c4)cc3)ccc2c1 ; c1ccc(-c2ccccc2-c2ccc(N(c3ccc(-c4cc5ccccc5c5ccccc45)cc3)c3ccc4oc5ccccc5c4c3)cc2)cc1] 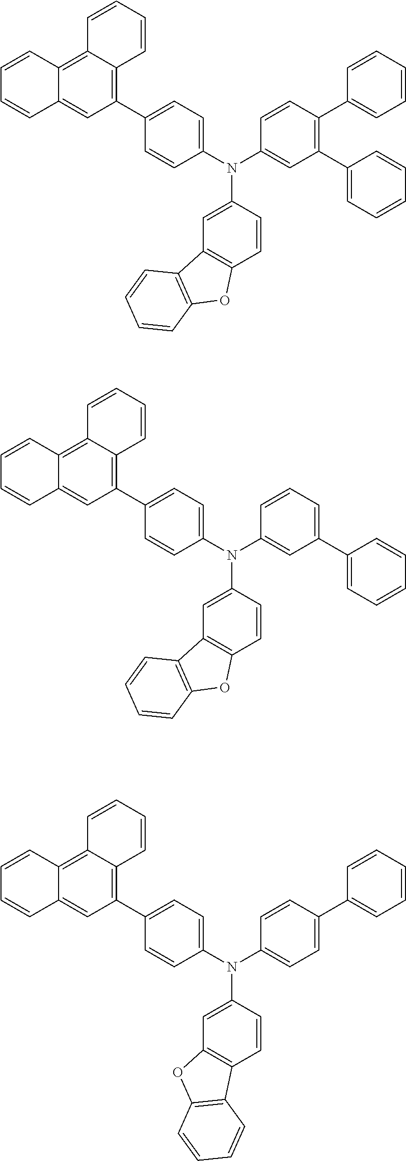 [SMILES: c1ccc(-c2ccc(N(c3ccc(-c4cc5ccccc5c5ccccc45)cc3)c3ccc4oc5ccccc5c4c3)cc2-c2ccccc2)cc1 ; c1ccc(-c2cccc(N(c3ccc(-c4cc5ccccc5c5ccccc45)cc3)c3ccc4oc5ccccc5c4c3)c2)cc1 ; c1ccc(-c2ccc(N(c3ccc(-c4cc5ccccc5c5ccccc45)cc3)c3ccc4c(c3)oc3ccccc34)cc2)cc1] 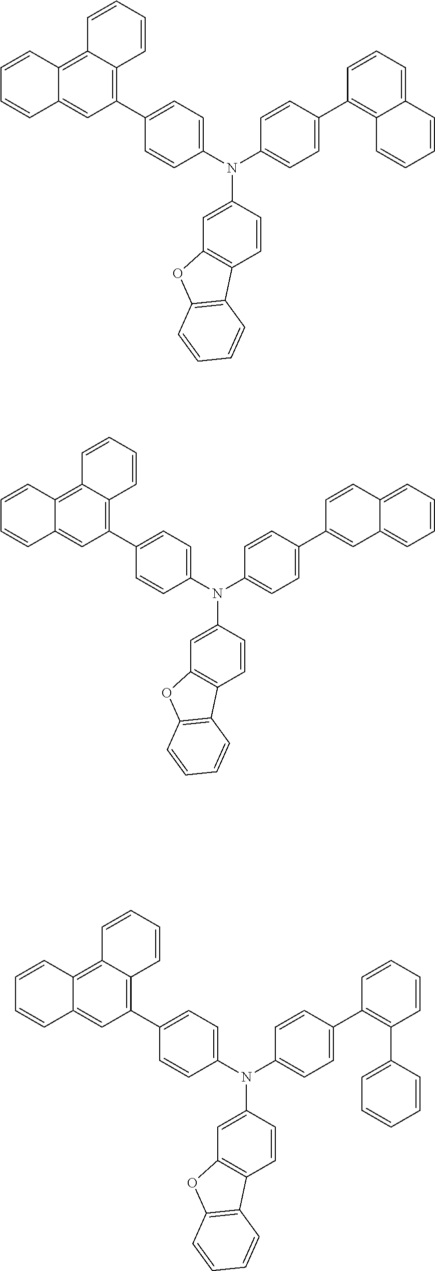 [SMILES: c1ccc2c(-c3ccc(N(c4ccc(-c5cc6ccccc6c6ccccc56)cc4)c4ccc5c(c4)oc4ccccc45)cc3)cccc2c1 ; c1ccc2cc(-c3ccc(N(c4ccc(-c5cc6ccccc6c6ccccc56)cc4)c4ccc5c(c4)oc4ccccc45)cc3)ccc2c1 ; c1ccc(-c2ccccc2-c2ccc(N(c3ccc(-c4cc5ccccc5c5ccccc45)cc3)c3ccc4c(c3)oc3ccccc34)cc2)cc1] 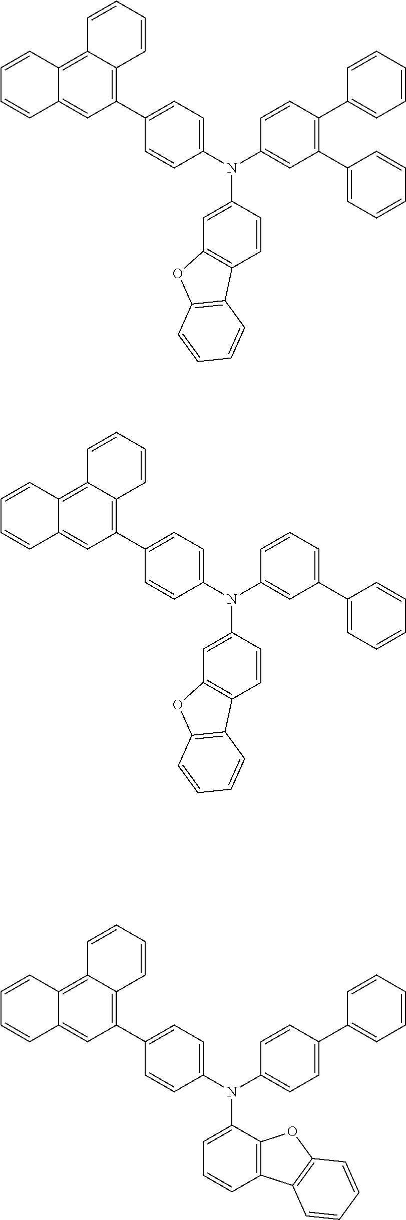 [SMILES: c1ccc(-c2ccc(N(c3ccc(-c4cc5ccccc5c5ccccc45)cc3)c3ccc4c(c3)oc3ccccc34)cc2-c2ccccc2)cc1 ; c1ccc(-c2cccc(N(c3ccc(-c4cc5ccccc5c5ccccc45)cc3)c3ccc4c(c3)oc3ccccc34)c2)cc1 ; c1ccc(-c2ccc(N(c3ccc(-c4cc5ccccc5c5ccccc45)cc3)c3cccc4c3oc3ccccc34)cc2)cc1] 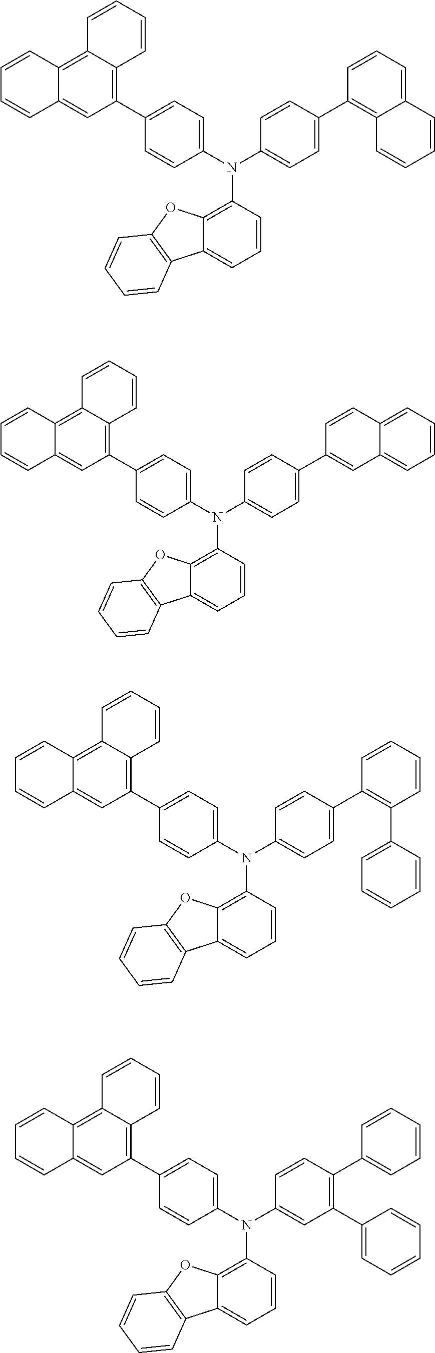 [SMILES: c1ccc2c(-c3ccc(N(c4ccc(-c5cc6ccccc6c6ccccc56)cc4)c4cccc5c4oc4ccccc45)cc3)cccc2c1 ; c1ccc2cc(-c3ccc(N(c4ccc(-c5cc6ccccc6c6ccccc56)cc4)c4cccc5c4oc4ccccc45)cc3)ccc2c1 ; c1ccc(-c2ccccc2-c2ccc(N(c3ccc(-c4cc5ccccc5c5ccccc45)cc3)c3cccc4c3oc3ccccc34)cc2)cc1 ; c1ccc(-c2ccc(N(c3ccc(-c4cc5ccccc5c5ccccc45)cc3)c3cccc4c3oc3ccccc34)cc2-c2ccccc2)cc1] 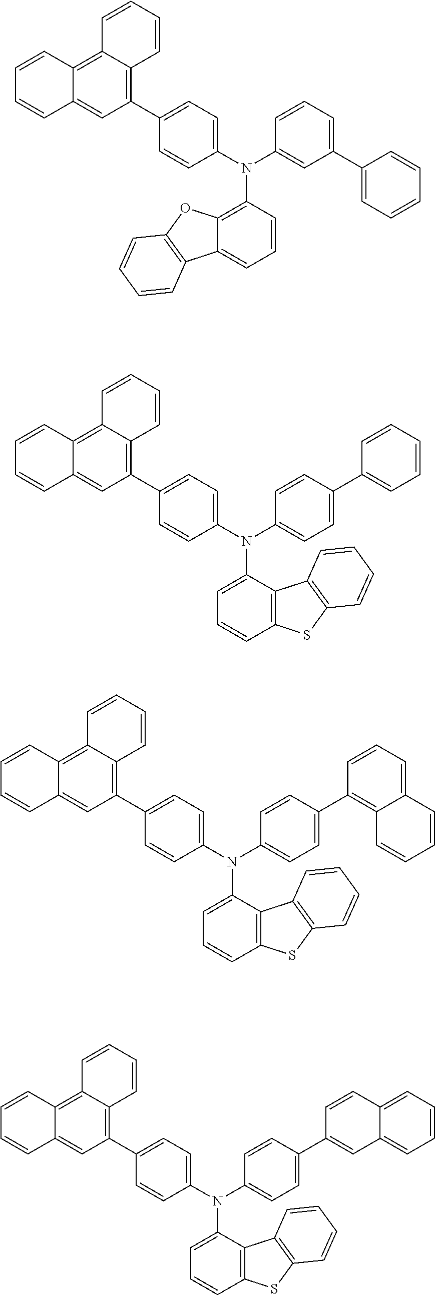 [SMILES: c1ccc(-c2cccc(N(c3ccc(-c4cc5ccccc5c5ccccc45)cc3)c3cccc4c3oc3ccccc34)c2)cc1 ; c1ccc(-c2ccc(N(c3ccc(-c4cc5ccccc5c5ccccc45)cc3)c3cccc4sc5ccccc5c34)cc2)cc1 ; c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1] 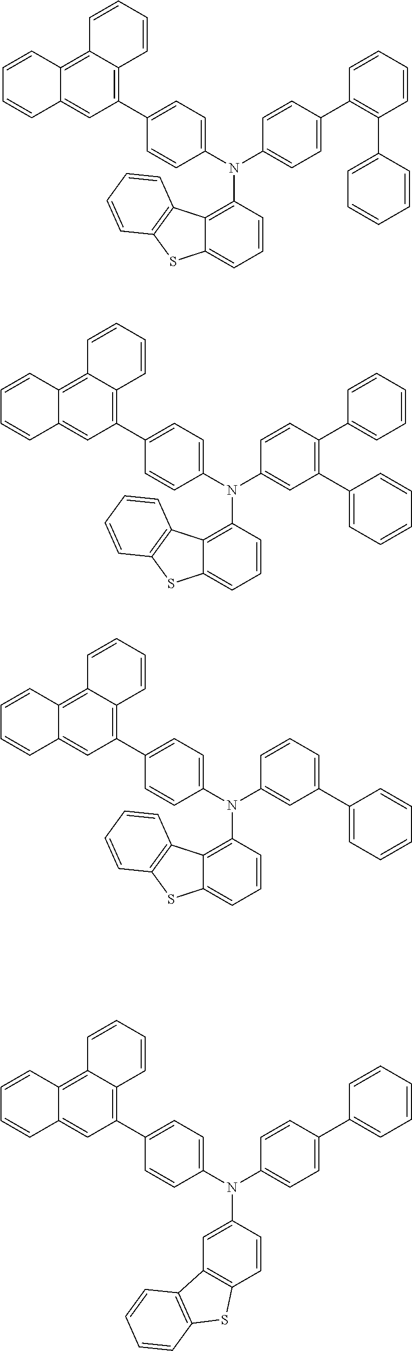 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)cc2-c2ccccc2)cc1 ; c1ccc(-c2cccc(N(c3ccc(-c4cc5ccccc5c5ccccc45)cc3)c3cccc4sc5ccccc5c34)c2)cc1 ; c1ccc(-c2ccc(N(c3ccc(-c4cc5ccccc5c5ccccc45)cc3)c3ccc4sc5ccccc5c4c3)cc2)cc1] 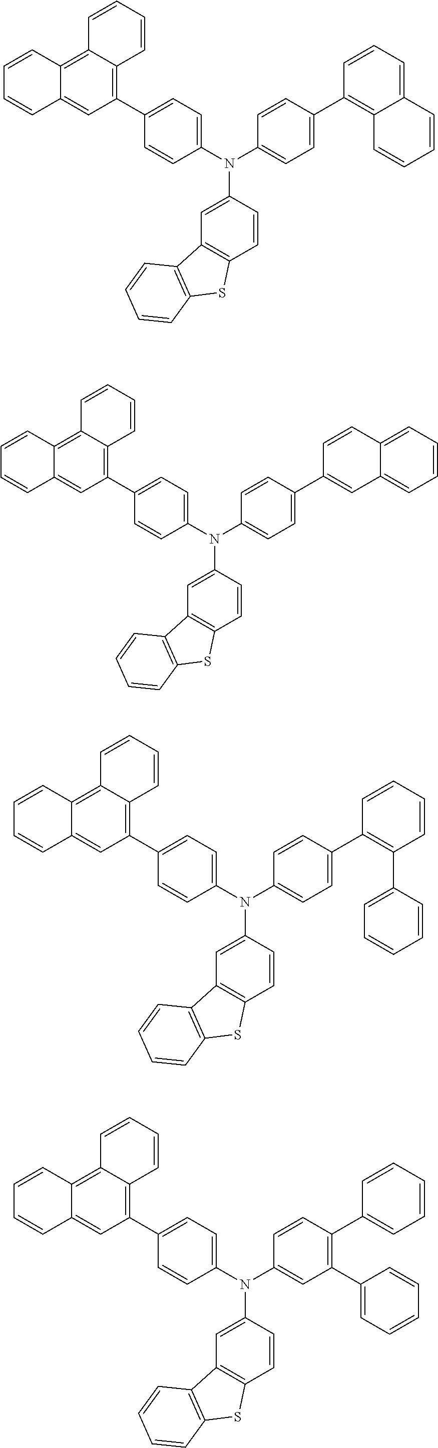 [SMILES: c1ccc2c(-c3ccc(N(c4ccc(-c5cc6ccccc6c6ccccc56)cc4)c4ccc5sc6ccccc6c5c4)cc3)cccc2c1 ; c1ccc2cc(-c3ccc(N(c4ccc(-c5cc6ccccc6c6ccccc56)cc4)c4ccc5sc6ccccc6c5c4)cc3)ccc2c1 ; c1ccc(-c2ccccc2-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4sc5ccccc5c4c3)cc2-c2ccccc2)cc1] 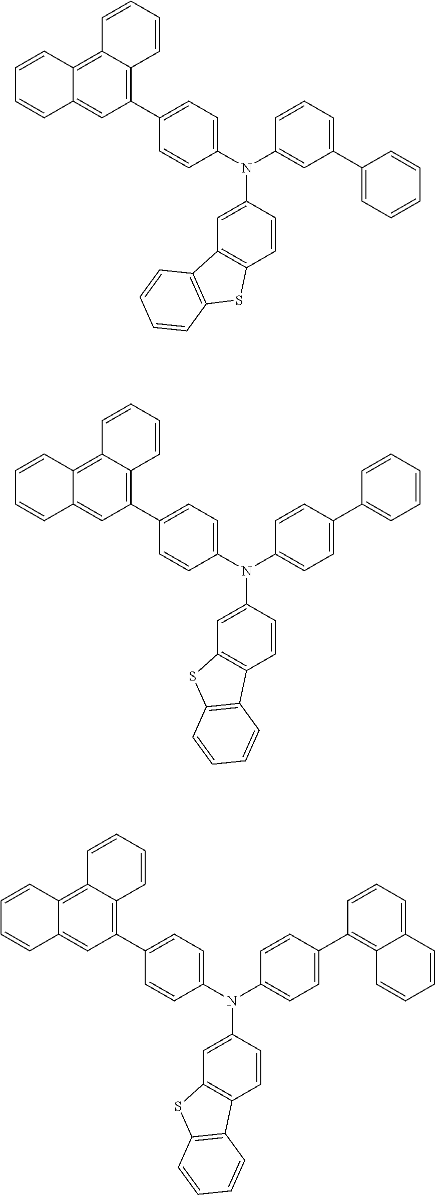 [SMILES: c1ccc(-c2cccc(N(c3ccc(-c4cc5ccccc5c5ccccc45)cc3)c3ccc4sc5ccccc5c4c3)c2)cc1 ; c1ccc(-c2ccc(N(c3ccc(-c4cc5ccccc5c5ccccc45)cc3)c3ccc4c(c3)sc3ccccc34)cc2)cc1 ; c1ccc2c(-c3ccc(N(c4ccc(-c5cc6ccccc6c6ccccc56)cc4)c4ccc5c(c4)sc4ccccc45)cc3)cccc2c1] 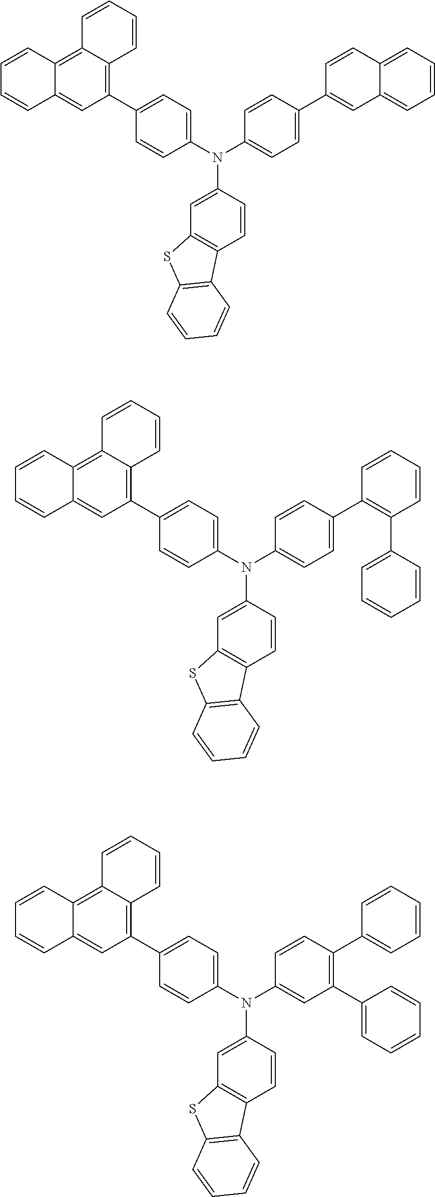 [SMILES: c1ccc2cc(-c3ccc(N(c4ccc(-c5cc6ccccc6c6ccccc56)cc4)c4ccc5c(c4)sc4ccccc45)cc3)ccc2c1 ; c1ccc(-c2ccccc2-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2-c2ccccc2)cc1] 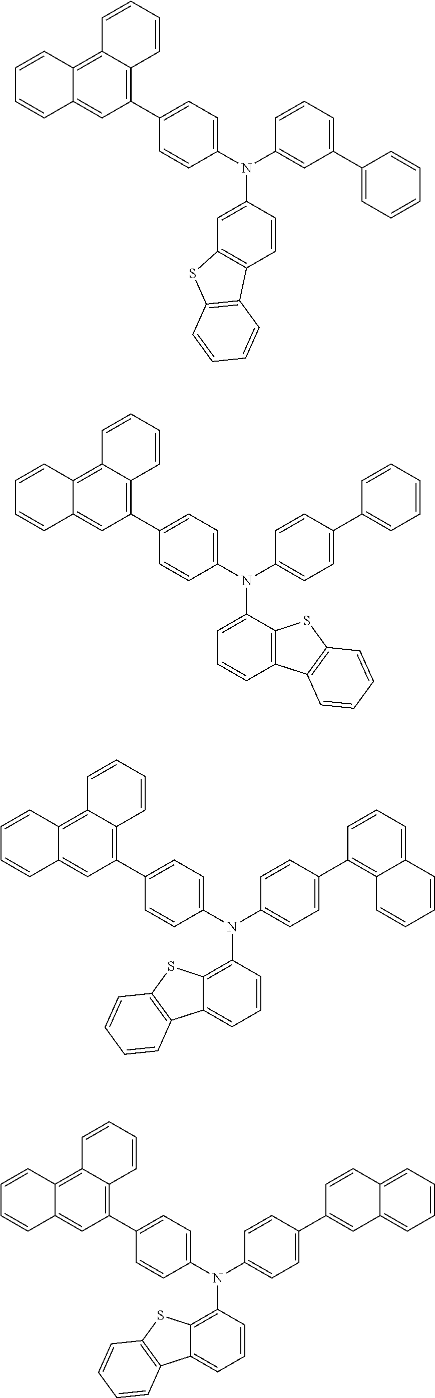 [SMILES: c1ccc(-c2cccc(N(c3ccc(-c4cc5ccccc5c5ccccc45)cc3)c3ccc4c(c3)sc3ccccc34)c2)cc1 ; c1ccc(-c2ccc(N(c3ccc(-c4cc5ccccc5c5ccccc45)cc3)c3cccc4c3sc3ccccc34)cc2)cc1 ; c1ccc2c(-c3ccc(N(c4ccc(-c5cc6ccccc6c6ccccc56)cc4)c4cccc5c4sc4ccccc45)cc3)cccc2c1 ; c1ccc2cc(-c3ccc(N(c4ccc(-c5cc6ccccc6c6ccccc56)cc4)c4cccc5c4sc4ccccc45)cc3)ccc2c1] 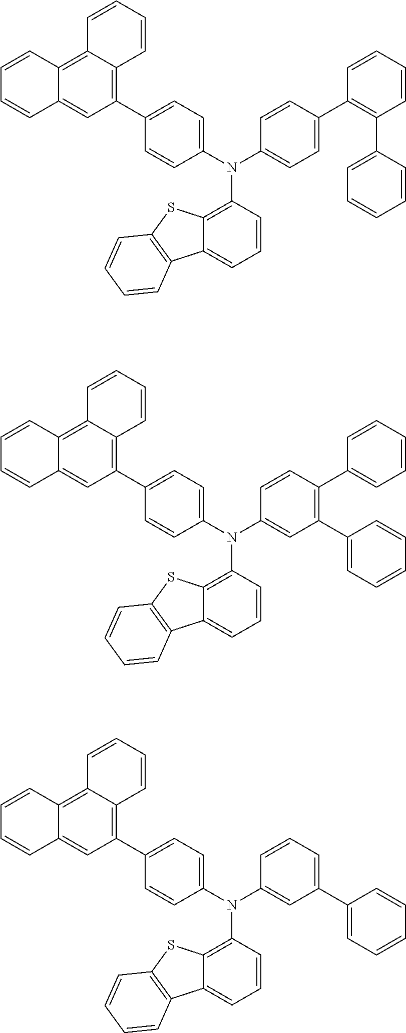 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)cc2-c2ccccc2)cc1 ; c1ccc(-c2cccc(N(c3ccc(-c4cc5ccccc5c5ccccc45)cc3)c3cccc4c3sc3ccccc34)c2)cc1] 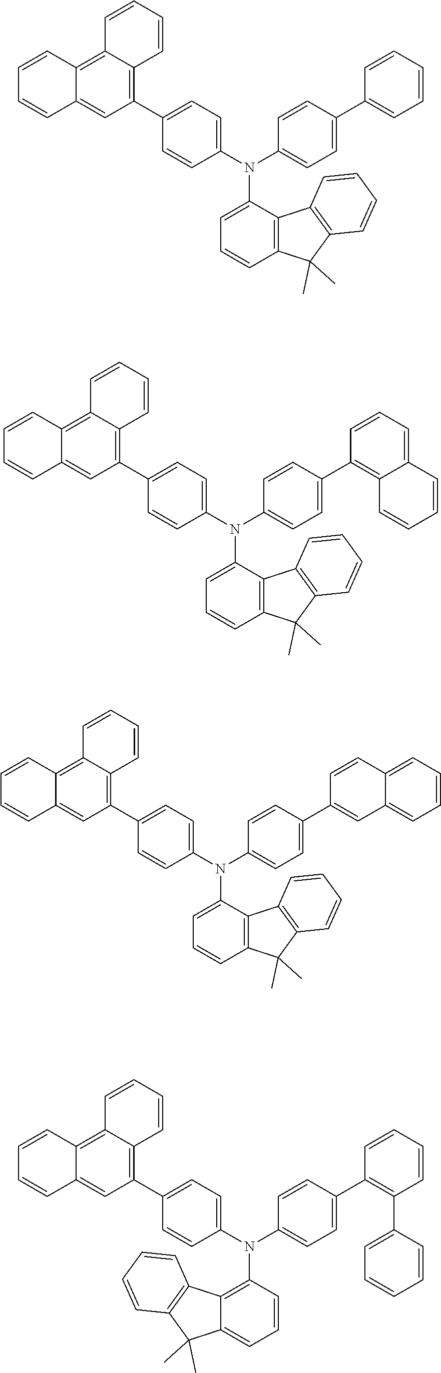 [SMILES: CC1(C)c2ccccc2-c2c(N(c3ccc(-c4ccccc4)cc3)c3ccc(-c4cc5ccccc5c5ccccc45)cc3)cccc21 ; CC1(C)c2ccccc2-c2c(N(c3ccc(-c4cccc5ccccc45)cc3)c3ccc(-c4cc5ccccc5c5ccccc45)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21 ; CC1(C)c2ccccc2-c2c(N(c3ccc(-c4ccccc4-c4ccccc4)cc3)c3ccc(-c4cc5ccccc5c5ccccc45)cc3)cccc21] 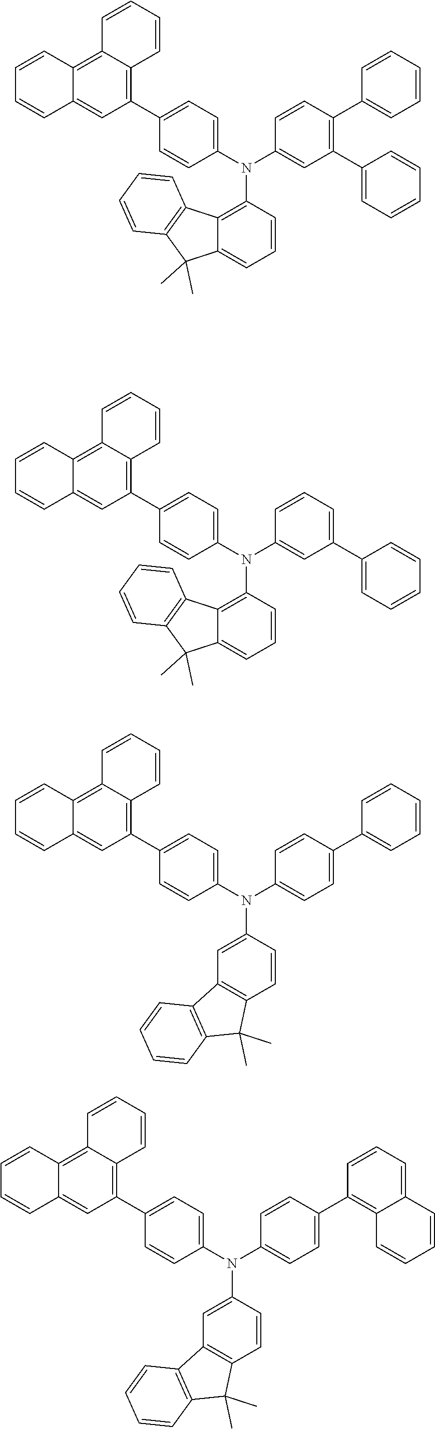 [SMILES: CC1(C)c2ccccc2-c2c(N(c3ccc(-c4cc5ccccc5c5ccccc45)cc3)c3ccc(-c4ccccc4)c(-c4ccccc4)c3)cccc21 ; CC1(C)c2ccccc2-c2c(N(c3ccc(-c4cc5ccccc5c5ccccc45)cc3)c3cccc(-c4ccccc4)c3)cccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4cccc5ccccc45)cc3)c3ccc(-c4cc5ccccc5c5ccccc45)cc3)ccc21] 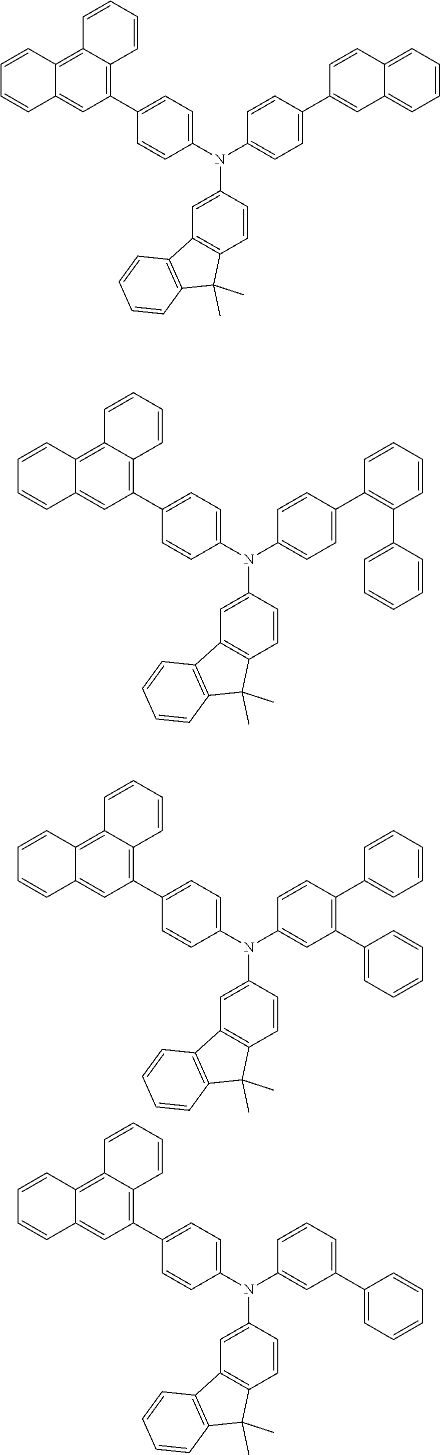 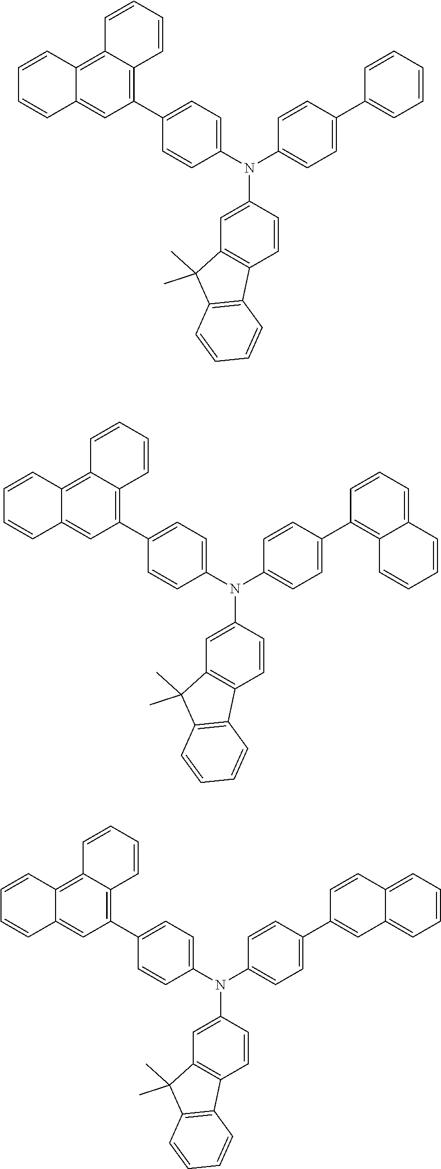 [SMILES: CC1(C)c2ccccc2-c2ccc(N(c3ccc(-c4ccccc4)cc3)c3ccc(-c4cc5ccccc5c5ccccc45)cc3)cc21 ; CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21] 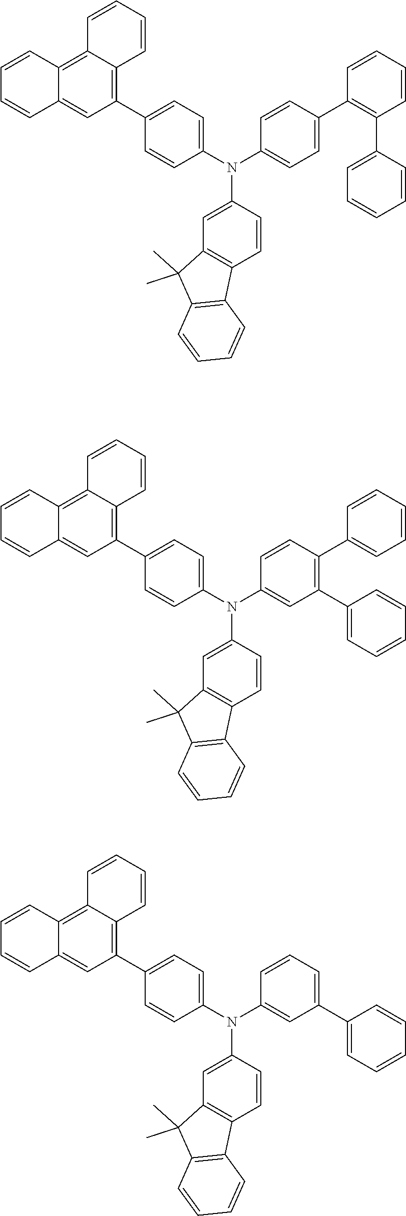 [SMILES: CC1(C)c2ccccc2-c2ccc(N(c3ccc(-c4ccccc4-c4ccccc4)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc(-c4ccccc4)c(-c4ccccc4)c3)cc21 ; CC1(C)c2ccccc2-c2ccc(N(c3ccc(-c4cc5ccccc5c5ccccc45)cc3)c3cccc(-c4ccccc4)c3)cc21] 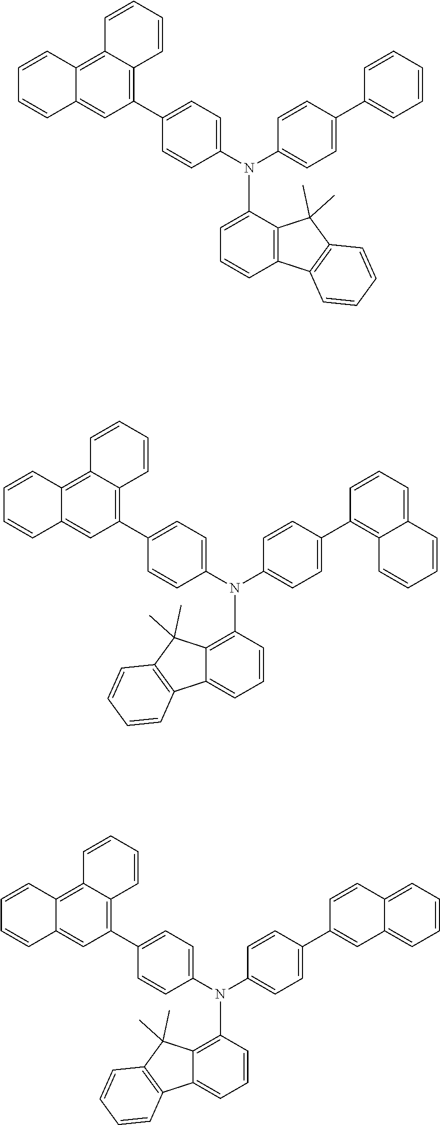 [SMILES: CC1(C)c2ccccc2-c2cccc(N(c3ccc(-c4ccccc4)cc3)c3ccc(-c4cc5ccccc5c5ccccc45)cc3)c21 ; CC1(C)c2ccccc2-c2cccc(N(c3ccc(-c4cccc5ccccc45)cc3)c3ccc(-c4cc5ccccc5c5ccccc45)cc3)c21 ; CC1(C)c2ccccc2-c2cccc(N(c3ccc(-c4ccc5ccccc5c4)cc3)c3ccc(-c4cc5ccccc5c5ccccc45)cc3)c21] 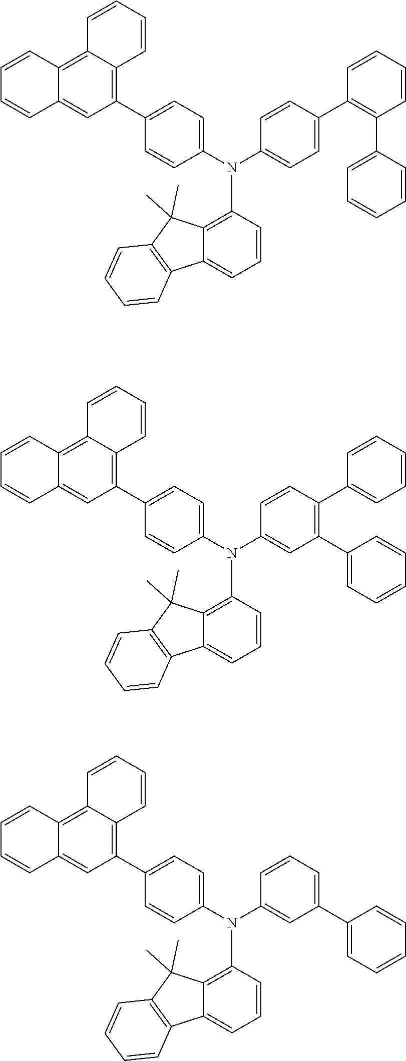 [SMILES: CC1(C)c2ccccc2-c2cccc(N(c3ccc(-c4ccccc4-c4ccccc4)cc3)c3ccc(-c4cc5ccccc5c5ccccc45)cc3)c21 ; CC1(C)c2ccccc2-c2cccc(N(c3ccc(-c4cc5ccccc5c5ccccc45)cc3)c3ccc(-c4ccccc4)c(-c4ccccc4)c3)c21 ; CC1(C)c2ccccc2-c2cccc(N(c3ccc(-c4cc5ccccc5c5ccccc45)cc3)c3cccc(-c4ccccc4)c3)c21] 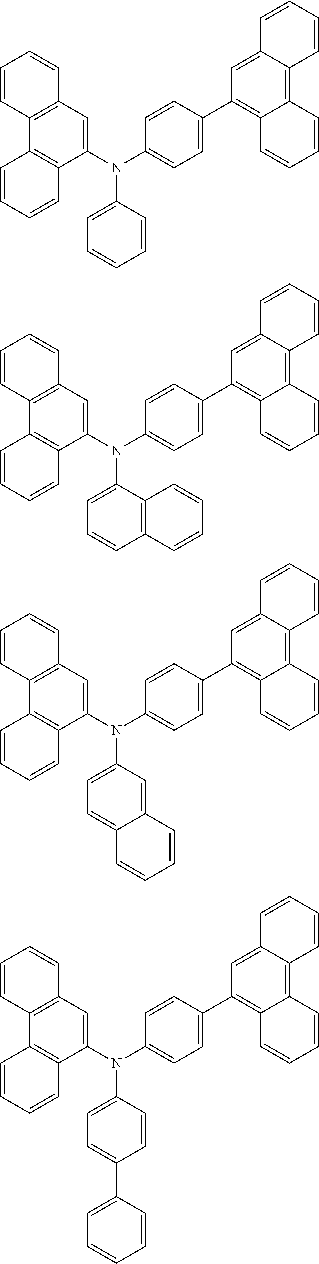 [SMILES: c1ccc(N(c2ccc(-c3cc4ccccc4c4ccccc34)cc2)c2cc3ccccc3c3ccccc23)cc1 ; c1ccc2c(N(c3ccc(-c4cc5ccccc5c5ccccc45)cc3)c3cc4ccccc4c4ccccc34)cccc2c1 ; c1ccc2cc(N(c3ccc(-c4cc5ccccc5c5ccccc45)cc3)c3cc4ccccc4c4ccccc34)ccc2c1 ; c1ccc(-c2ccc(N(c3ccc(-c4cc5ccccc5c5ccccc45)cc3)c3cc4ccccc4c4ccccc34)cc2)cc1] 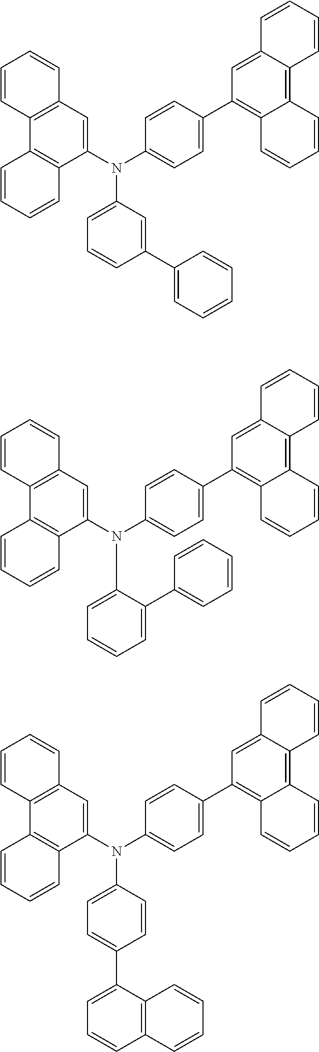 [SMILES: c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1 ; c1ccc2c(-c3ccc(N(c4ccc(-c5cc6ccccc6c6ccccc56)cc4)c4cc5ccccc5c5ccccc45)cc3)cccc2c1] 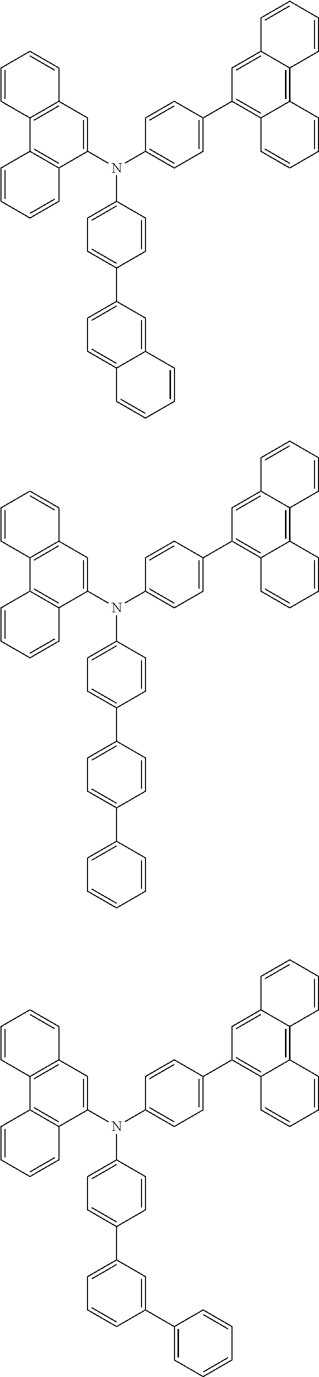 [SMILES: c1ccc2cc(-c3ccc(N(c4ccc(-c5cc6ccccc6c6ccccc56)cc4)c4cc5ccccc5c5ccccc45)cc3)ccc2c1 ; c1ccc(-c2ccc(-c3ccc(N(c4ccc(-c5cc6ccccc6c6ccccc56)cc4)c4cc5ccccc5c5ccccc45)cc3)cc2)cc1 ; c1ccc(-c2cccc(-c3ccc(N(c4ccc(-c5cc6ccccc6c6ccccc56)cc4)c4cc5ccccc5c5ccccc45)cc3)c2)cc1] 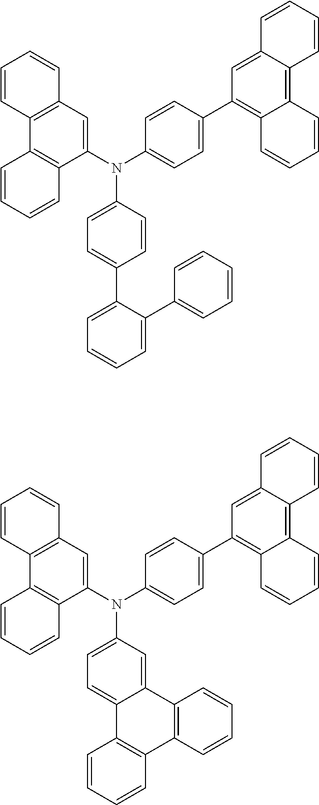 [SMILES: c1ccc(-c2ccccc2-c2ccc(N(c3ccc(-c4cc5ccccc5c5ccccc45)cc3)c3cc4ccccc4c4ccccc34)cc2)cc1 ; c1ccc2c(c1)cc(-c1ccc(N(c3ccc4c5ccccc5c5ccccc5c4c3)c3cc4ccccc4c4ccccc34)cc1)c1ccccc12] 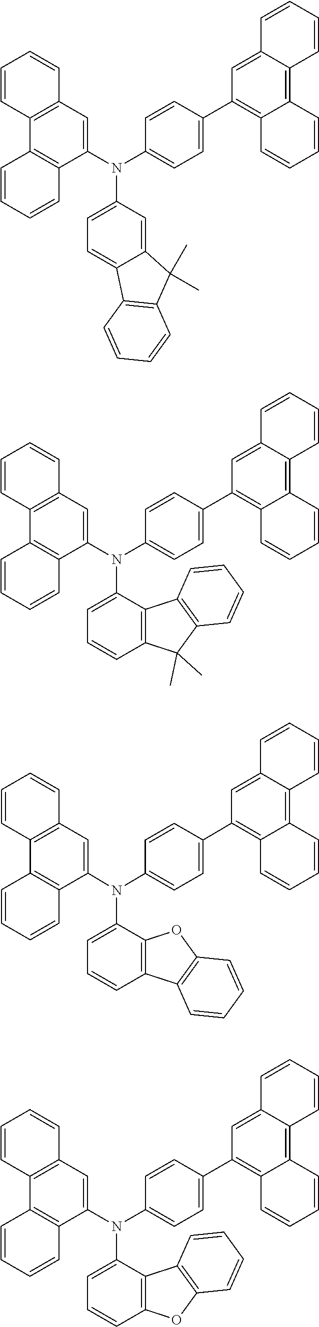 [SMILES: CC1(C)c2ccccc2-c2ccc(N(c3ccc(-c4cc5ccccc5c5ccccc45)cc3)c3cc4ccccc4c4ccccc34)cc21 ; CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12 ; c1ccc2c(c1)cc(-c1ccc(N(c3cc4ccccc4c4ccccc34)c3cccc4oc5ccccc5c34)cc1)c1ccccc12] 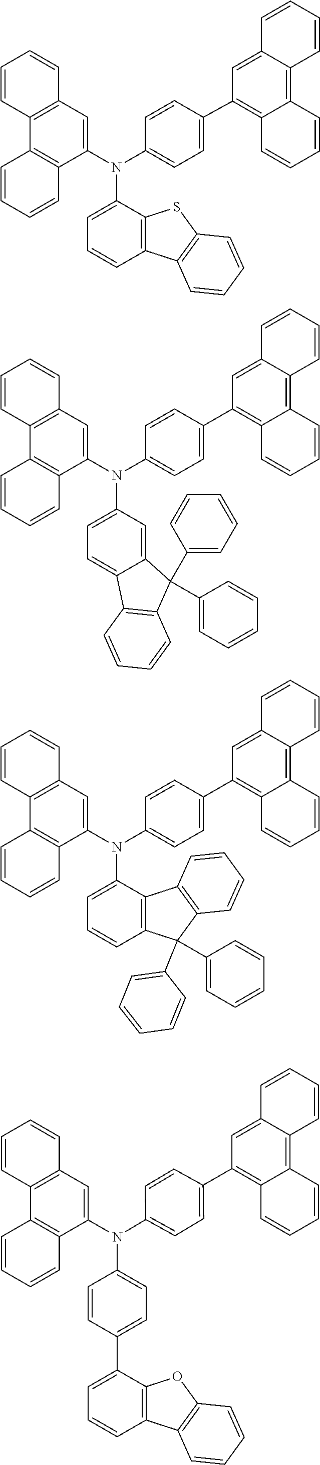 [SMILES: c1ccc2c(c1)cc(-c1ccc(N(c3cc4ccccc4c4ccccc34)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4cc5ccccc5c5ccccc45)cc32)cc1 ; c1ccc(C2(c3ccccc3)c3ccccc3-c3c(N(c4ccc(-c5cc6ccccc6c6ccccc56)cc4)c4cc5ccccc5c5ccccc45)cccc32)cc1 ; c1ccc2c(c1)cc(-c1ccc(N(c3ccc(-c4cccc5c4oc4ccccc45)cc3)c3cc4ccccc4c4ccccc34)cc1)c1ccccc12] 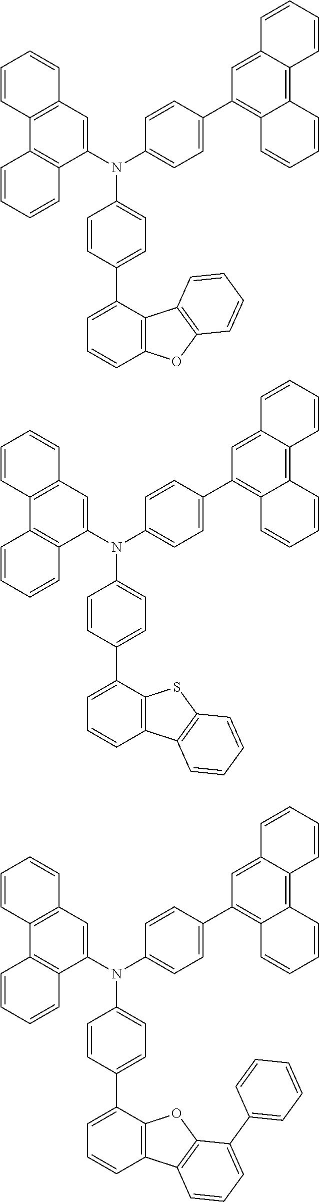 [SMILES: c1ccc2c(c1)cc(-c1ccc(N(c3ccc(-c4cccc5oc6ccccc6c45)cc3)c3cc4ccccc4c4ccccc34)cc1)c1ccccc12 ; c1ccc2c(c1)cc(-c1ccc(N(c3ccc(-c4cccc5c4sc4ccccc45)cc3)c3cc4ccccc4c4ccccc34)cc1)c1ccccc12 ; c1ccc(-c2cccc3c2oc2c(-c4ccc(N(c5ccc(-c6cc7ccccc7c7ccccc67)cc5)c5cc6ccccc6c6ccccc56)cc4)cccc23)cc1] 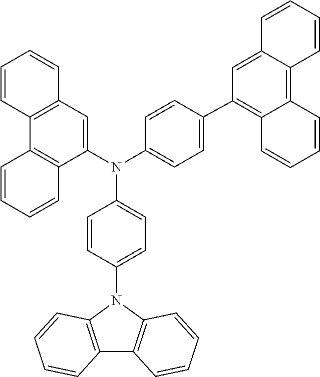 [SMILES: c1ccc2c(c1)cc(-c1ccc(N(c3ccc(-n4c5ccccc5c5ccccc54)cc3)c3cc4ccccc4c4ccccc34)cc1)c1ccccc12] 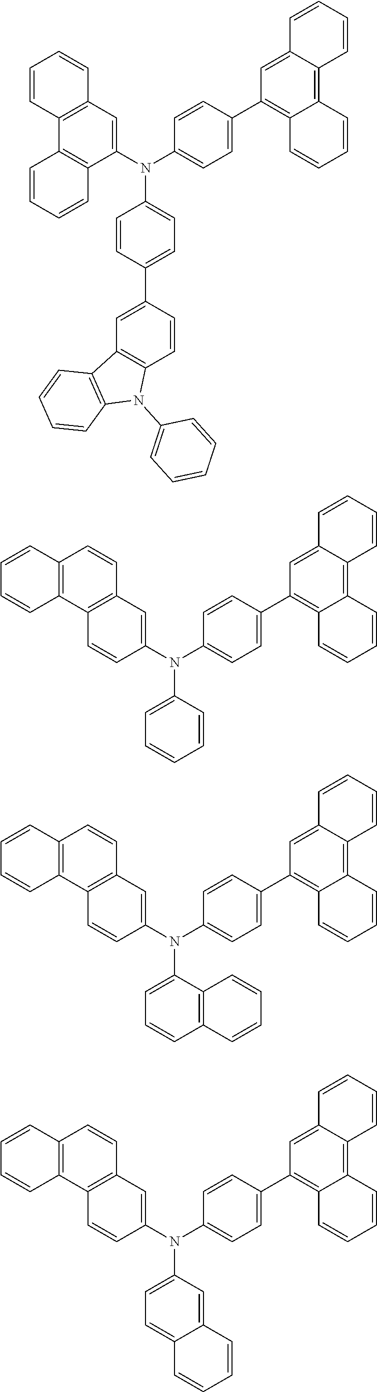 [SMILES: c1ccc(-n2c3ccccc3c3cc(-c4ccc(N(c5ccc(-c6cc7ccccc7c7ccccc67)cc5)c5cc6ccccc6c6ccccc56)cc4)ccc32)cc1 ; c1ccc(N(c2ccc(-c3cc4ccccc4c4ccccc34)cc2)c2ccc3c(ccc4ccccc43)c2)cc1 ; c1ccc2c(N(c3ccc(-c4cc5ccccc5c5ccccc45)cc3)c3ccc4c(ccc5ccccc54)c3)cccc2c1 ; c1ccc2cc(N(c3ccc(-c4cc5ccccc5c5ccccc45)cc3)c3ccc4c(ccc5ccccc54)c3)ccc2c1] 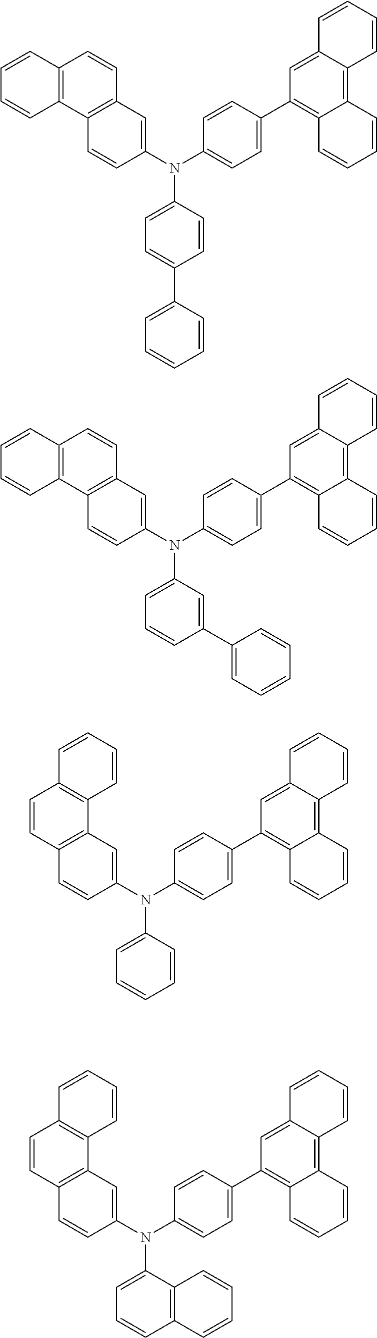 [SMILES: c1ccc(-c2ccc(N(c3ccc(-c4cc5ccccc5c5ccccc45)cc3)c3ccc4c(ccc5ccccc54)c3)cc2)cc1 ; c1ccc(-c2cccc(N(c3ccc(-c4cc5ccccc5c5ccccc45)cc3)c3ccc4c(ccc5ccccc54)c3)c2)cc1 ; c1ccc(N(c2ccc(-c3cc4ccccc4c4ccccc34)cc2)c2ccc3ccc4ccccc4c3c2)cc1 ; c1ccc2c(N(c3ccc(-c4cc5ccccc5c5ccccc45)cc3)c3ccc4ccc5ccccc5c4c3)cccc2c1] 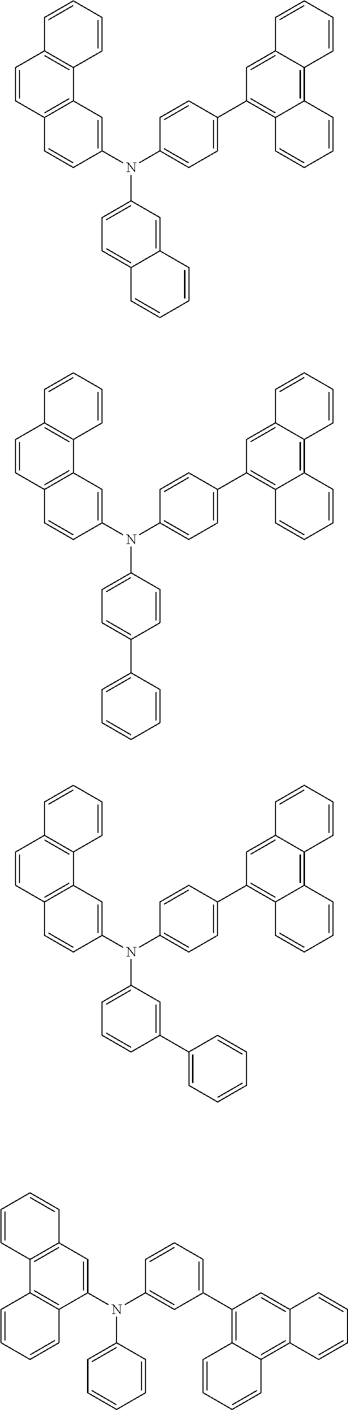 [SMILES: c1ccc2cc(N(c3ccc(-c4cc5ccccc5c5ccccc45)cc3)c3ccc4ccc5ccccc5c4c3)ccc2c1 ; c1ccc(-c2ccc(N(c3ccc(-c4cc5ccccc5c5ccccc45)cc3)c3ccc4ccc5ccccc5c4c3)cc2)cc1 ; c1ccc(-c2cccc(N(c3ccc(-c4cc5ccccc5c5ccccc45)cc3)c3ccc4ccc5ccccc5c4c3)c2)cc1 ; c1ccc(N(c2cccc(-c3cc4ccccc4c4ccccc34)c2)c2cc3ccccc3c3ccccc23)cc1] 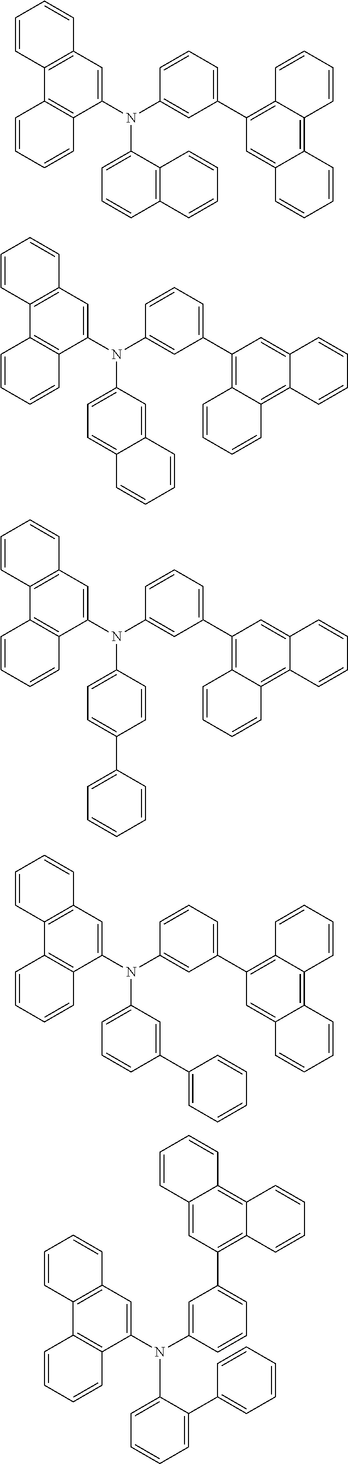 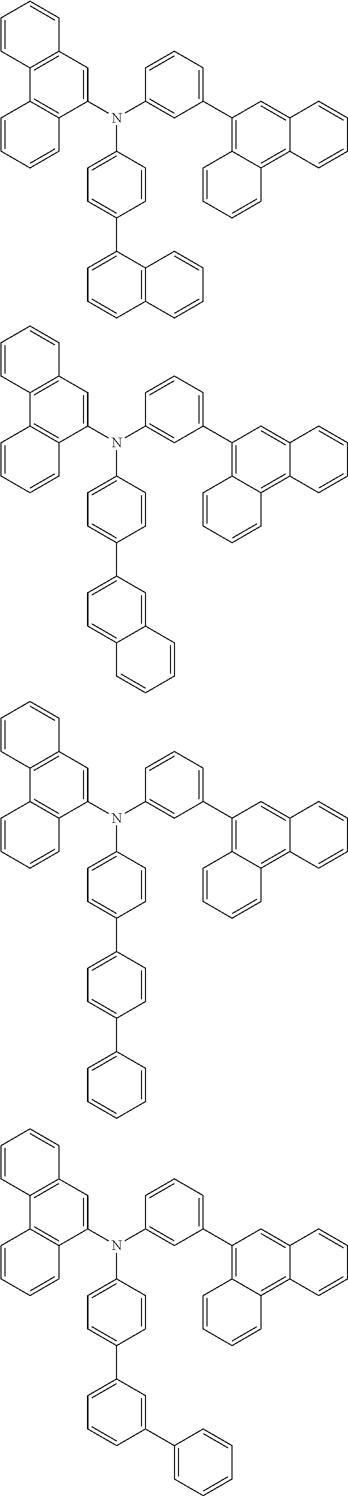 [SMILES: c1cc(-c2cc3ccccc3c3ccccc23)cc(N(c2ccc(-c3cccc4ccccc34)cc2)c2cc3ccccc3c3ccccc23)c1 ; c1cc(-c2cc3ccccc3c3ccccc23)cc(N(c2ccc(-c3ccc4ccccc4c3)cc2)c2cc3ccccc3c3ccccc23)c1 ; c1ccc(-c2ccc(-c3ccc(N(c4cccc(-c5cc6ccccc6c6ccccc56)c4)c4cc5ccccc5c5ccccc45)cc3)cc2)cc1 ; c1ccc(-c2cccc(-c3ccc(N(c4cccc(-c5cc6ccccc6c6ccccc56)c4)c4cc5ccccc5c5ccccc45)cc3)c2)cc1] 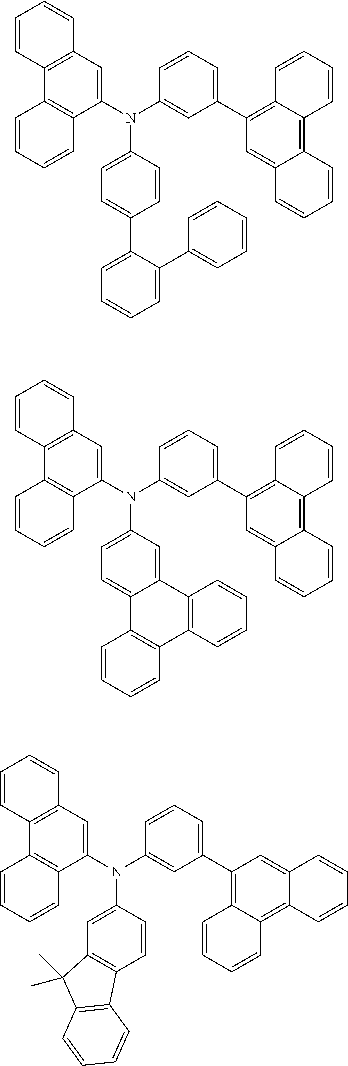 [SMILES: c1ccc(-c2ccccc2-c2ccc(N(c3cccc(-c4cc5ccccc5c5ccccc45)c3)c3cc4ccccc4c4ccccc34)cc2)cc1 ; c1cc(-c2cc3ccccc3c3ccccc23)cc(N(c2ccc3c4ccccc4c4ccccc4c3c2)c2cc3ccccc3c3ccccc23)c1 ; CC1(C)c2ccccc2-c2ccc(N(c3cccc(-c4cc5ccccc5c5ccccc45)c3)c3cc4ccccc4c4ccccc34)cc21] 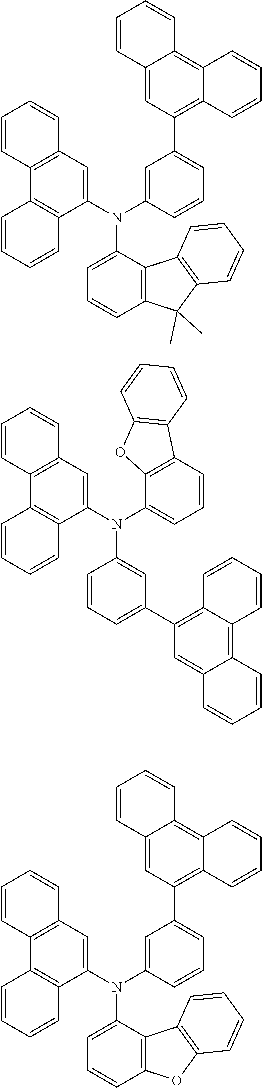 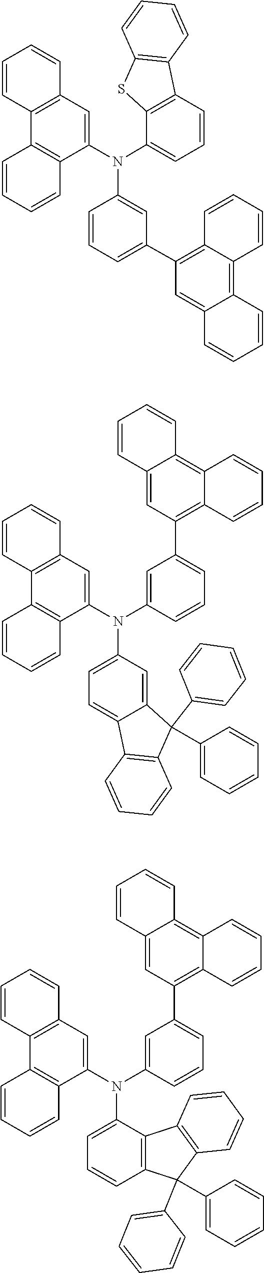 [SMILES: c1cc(-c2cc3ccccc3c3ccccc23)cc(N(c2cc3ccccc3c3ccccc23)c2cccc3c2sc2ccccc23)c1 ; c1ccc(C2(c3ccccc3)c3ccccc3-c3ccc(N(c4cccc(-c5cc6ccccc6c6ccccc56)c4)c4cc5ccccc5c5ccccc45)cc32)cc1 ; c1ccc(C2(c3ccccc3)c3ccccc3-c3c(N(c4cccc(-c5cc6ccccc6c6ccccc56)c4)c4cc5ccccc5c5ccccc45)cccc32)cc1] 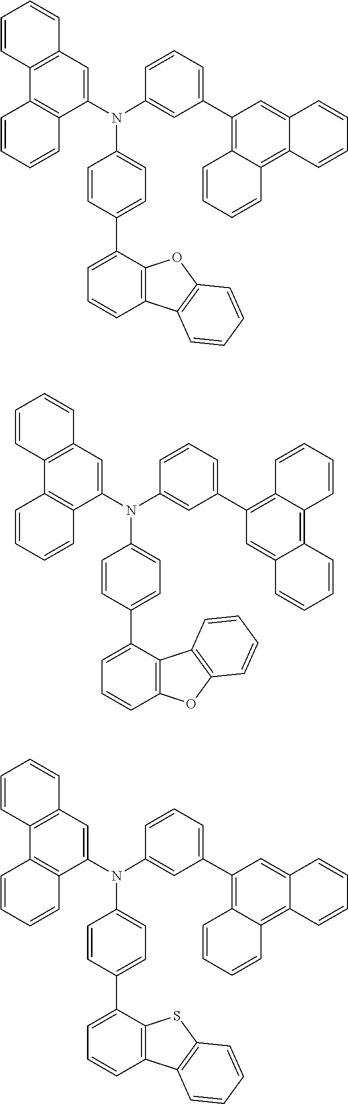 [SMILES: c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1 ; c1cc(-c2cc3ccccc3c3ccccc23)cc(N(c2ccc(-c3cccc4c3sc3ccccc34)cc2)c2cc3ccccc3c3ccccc23)c1] 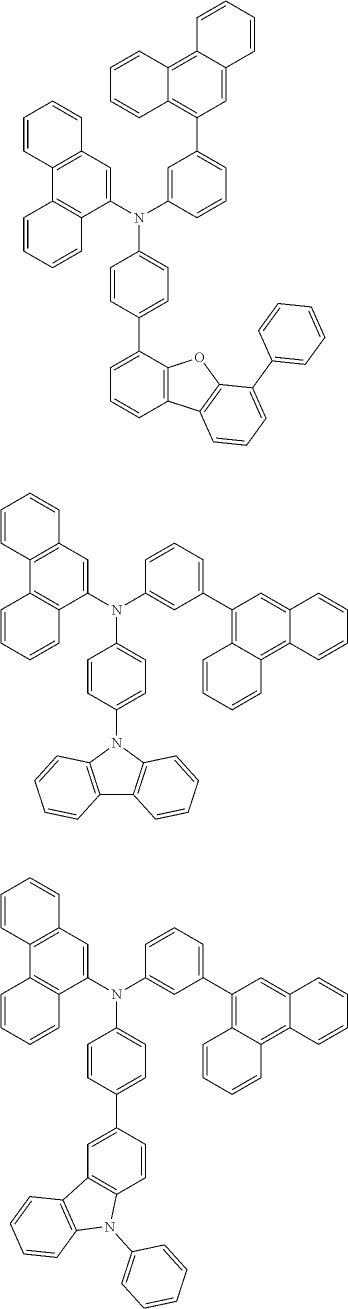 [SMILES: c1ccc(-c2cccc3c2oc2c(-c4ccc(N(c5cccc(-c6cc7ccccc7c7ccccc67)c5)c5cc6ccccc6c6ccccc56)cc4)cccc23)cc1 ; c1cc(-c2cc3ccccc3c3ccccc23)cc(N(c2ccc(-n3c4ccccc4c4ccccc43)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1] 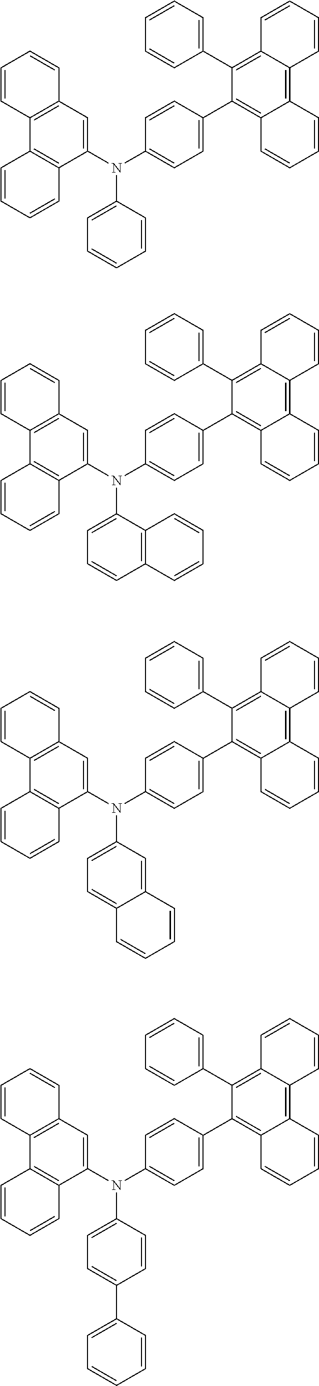 [SMILES: c1ccc(-c2c(-c3ccc(N(c4ccccc4)c4cc5ccccc5c5ccccc45)cc3)c3ccccc3c3ccccc23)cc1 ; c1ccc(-c2c(-c3ccc(N(c4cccc5ccccc45)c4cc5ccccc5c5ccccc45)cc3)c3ccccc3c3ccccc23)cc1 ; c1ccc(-c2c(-c3ccc(N(c4ccc5ccccc5c4)c4cc5ccccc5c5ccccc45)cc3)c3ccccc3c3ccccc23)cc1 ; c1ccc(-c2ccc(N(c3ccc(-c4c(-c5ccccc5)c5ccccc5c5ccccc45)cc3)c3cc4ccccc4c4ccccc34)cc2)cc1] 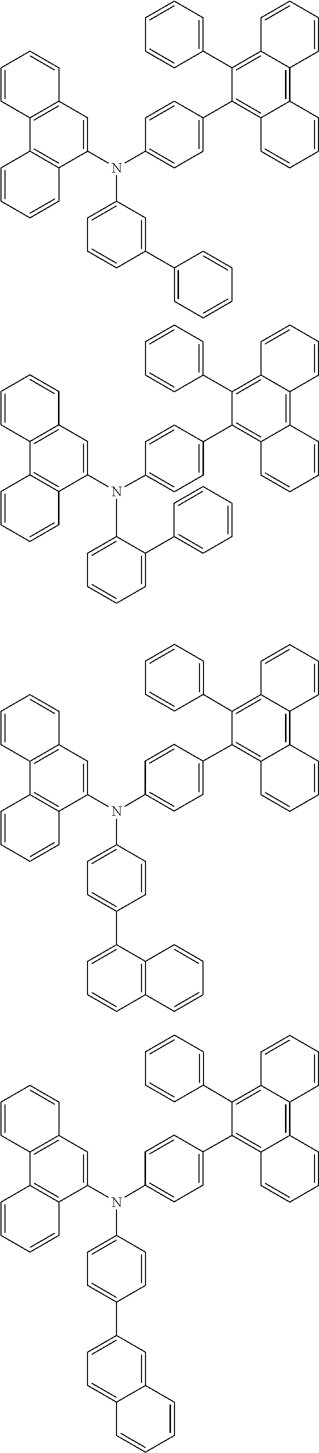 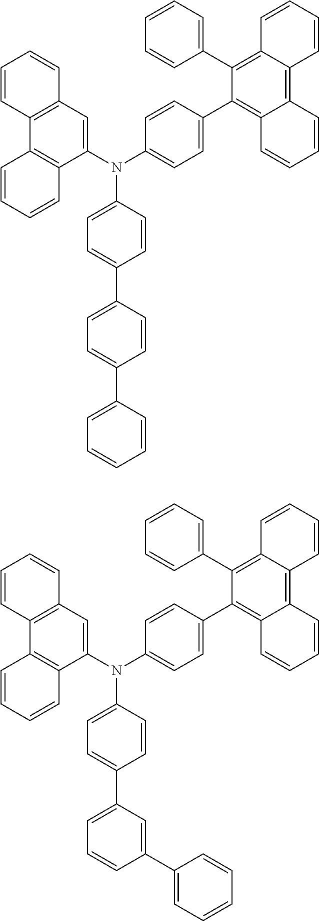 [SMILES: c1ccc(-c2ccc(-c3ccc(N(c4ccc(-c5c(-c6ccccc6)c6ccccc6c6ccccc56)cc4)c4cc5ccccc5c5ccccc45)cc3)cc2)cc1 ; c1ccc(-c2cccc(-c3ccc(N(c4ccc(-c5c(-c6ccccc6)c6ccccc6c6ccccc56)cc4)c4cc5ccccc5c5ccccc45)cc3)c2)cc1] 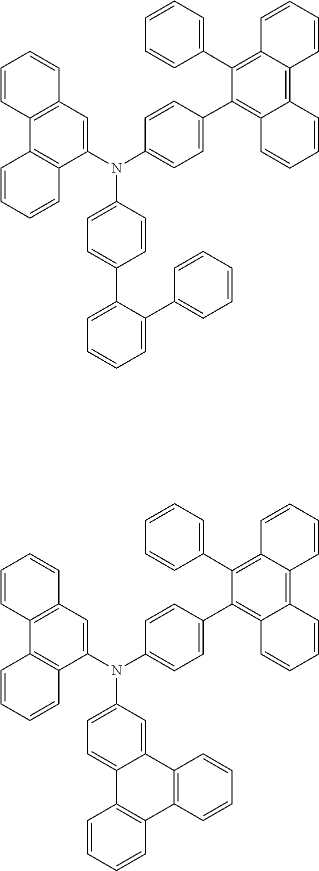 [SMILES: c1ccc(-c2ccccc2-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc5c6ccccc6c6ccccc6c5c4)c4cc5ccccc5c5ccccc45)cc3)c3ccccc3c3ccccc23)cc1] 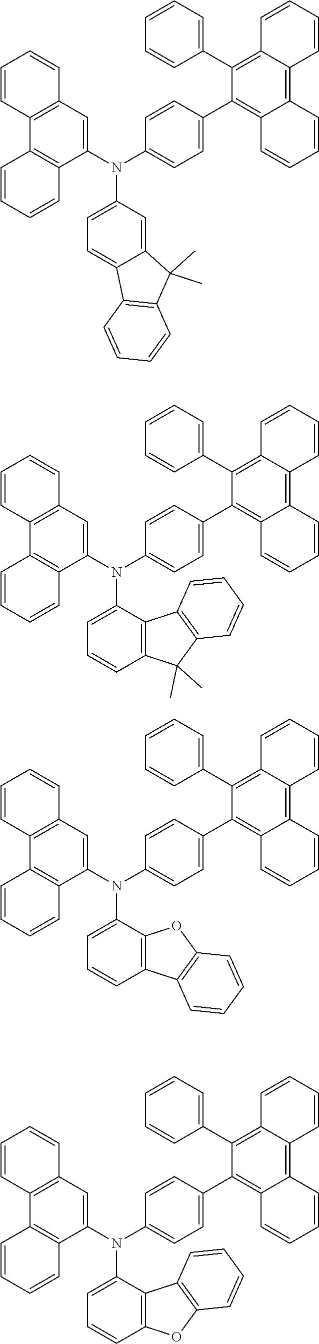 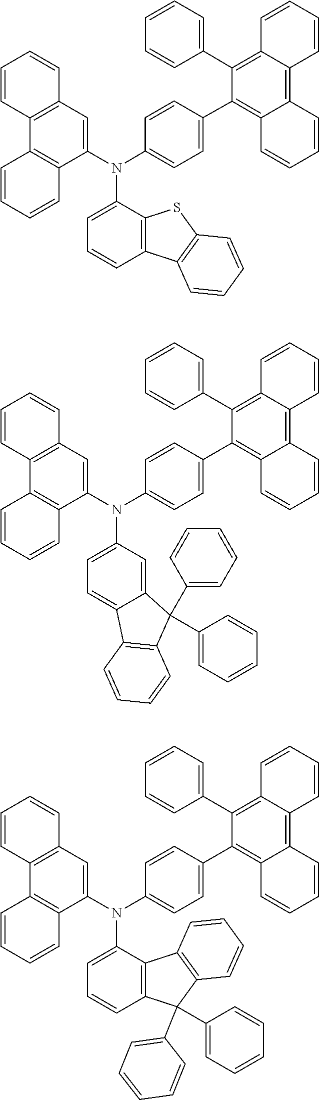 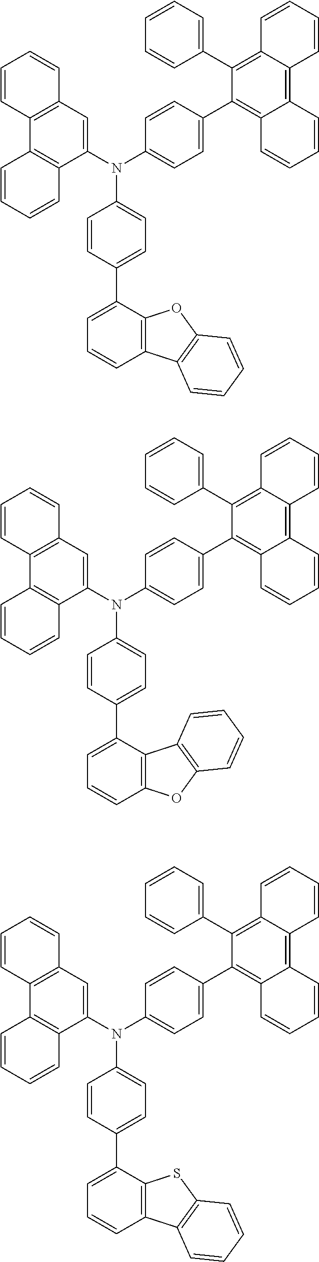 [SMILES: c1ccc(-c2c(-c3ccc(N(c4ccc(-c5cccc6c5oc5ccccc56)cc4)c4cc5ccccc5c5ccccc45)cc3)c3ccccc3c3ccccc23)cc1 ; c1ccc(-c2c(-c3ccc(N(c4ccc(-c5cccc6oc7ccccc7c56)cc4)c4cc5ccccc5c5ccccc45)cc3)c3ccccc3c3ccccc23)cc1 ; c1ccc(-c2c(-c3ccc(N(c4ccc(-c5cccc6c5sc5ccccc56)cc4)c4cc5ccccc5c5ccccc45)cc3)c3ccccc3c3ccccc23)cc1] 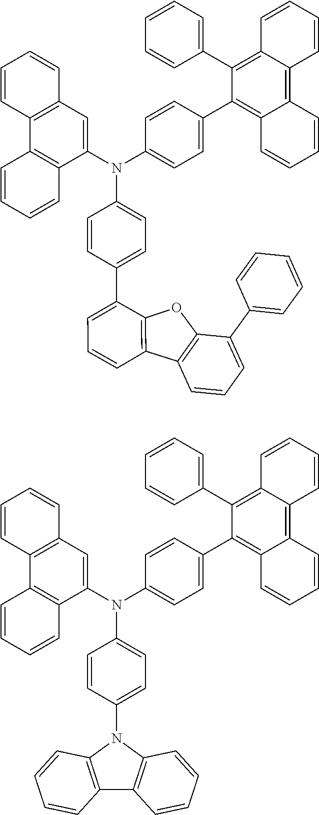 [SMILES: c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1] 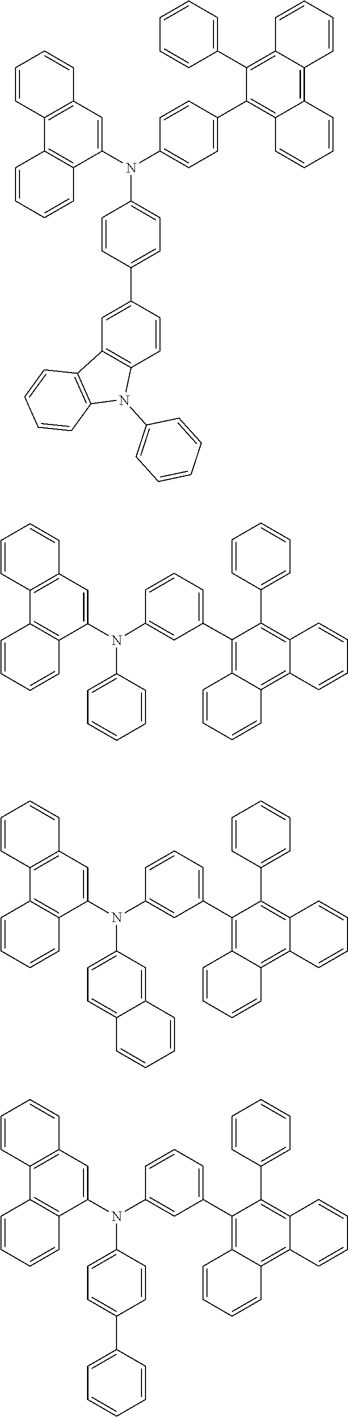 [SMILES: c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1 ; c1ccc(-c2ccc(N(c3cccc(-c4c(-c5ccccc5)c5ccccc5c5ccccc45)c3)c3cc4ccccc4c4ccccc34)cc2)cc1] 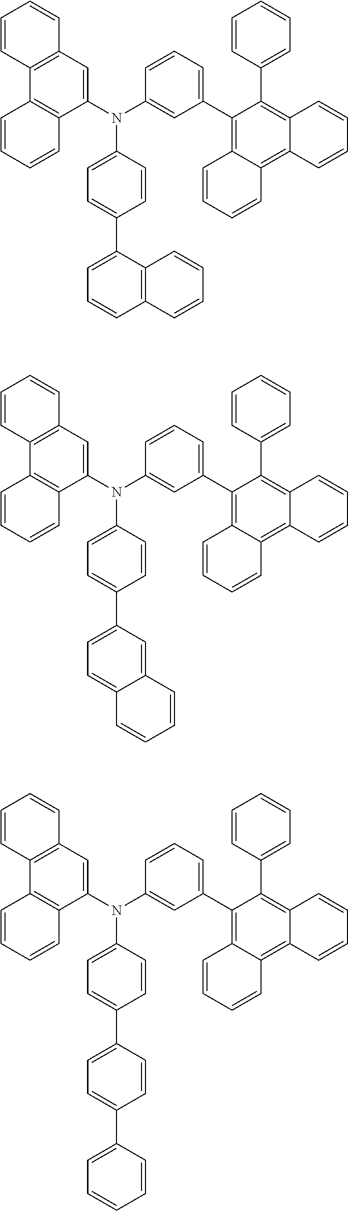 [SMILES: c1ccc(-c2c(-c3cccc(N(c4ccc(-c5cccc6ccccc56)cc4)c4cc5ccccc5c5ccccc45)c3)c3ccccc3c3ccccc23)cc1 ; c1ccc(-c2c(-c3cccc(N(c4ccc(-c5ccc6ccccc6c5)cc4)c4cc5ccccc5c5ccccc45)c3)c3ccccc3c3ccccc23)cc1 ; c1ccc(-c2ccc(-c3ccc(N(c4cccc(-c5c(-c6ccccc6)c6ccccc6c6ccccc56)c4)c4cc5ccccc5c5ccccc45)cc3)cc2)cc1] 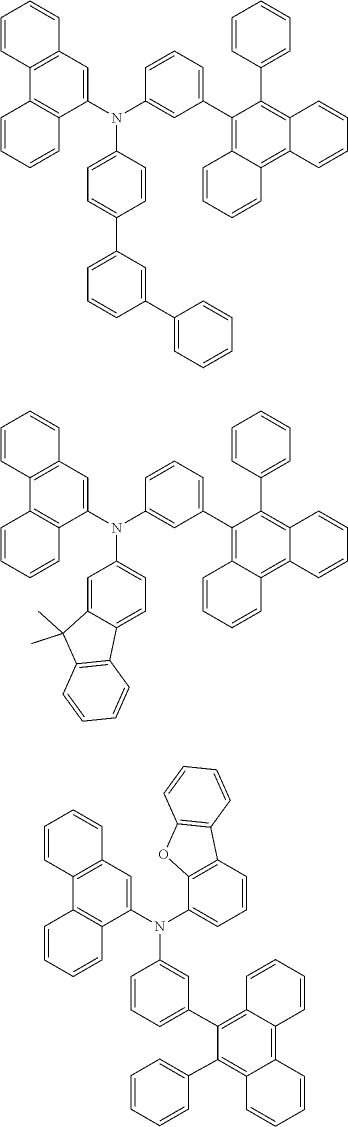 [SMILES: c1ccc(-c2cccc(-c3ccc(N(c4cccc(-c5c(-c6ccccc6)c6ccccc6c6ccccc56)c4)c4cc5ccccc5c5ccccc45)cc3)c2)cc1 ; CC1(C)c2ccccc2-c2ccc(N(c3cccc(-c4c(-c5ccccc5)c5ccccc5c5ccccc45)c3)c3cc4ccccc4c4ccccc34)cc21 ; c1ccc(-c2c(-c3cccc(N(c4cc5ccccc5c5ccccc45)c4cccc5c4oc4ccccc45)c3)c3ccccc3c3ccccc23)cc1] 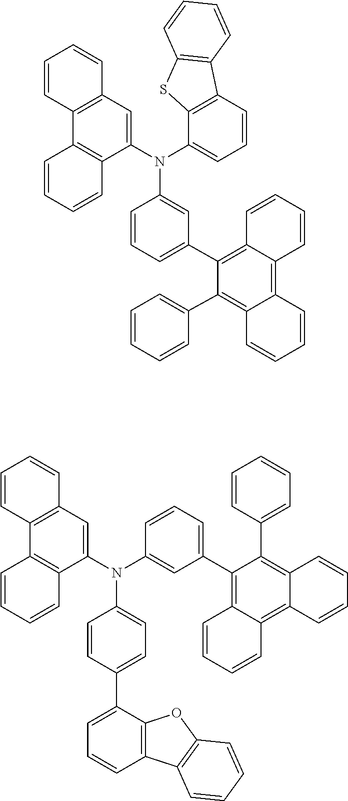 [SMILES: c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3cccc(N(c4ccc(-c5cccc6c5oc5ccccc56)cc4)c4cc5ccccc5c5ccccc45)c3)c3ccccc3c3ccccc23)cc1] 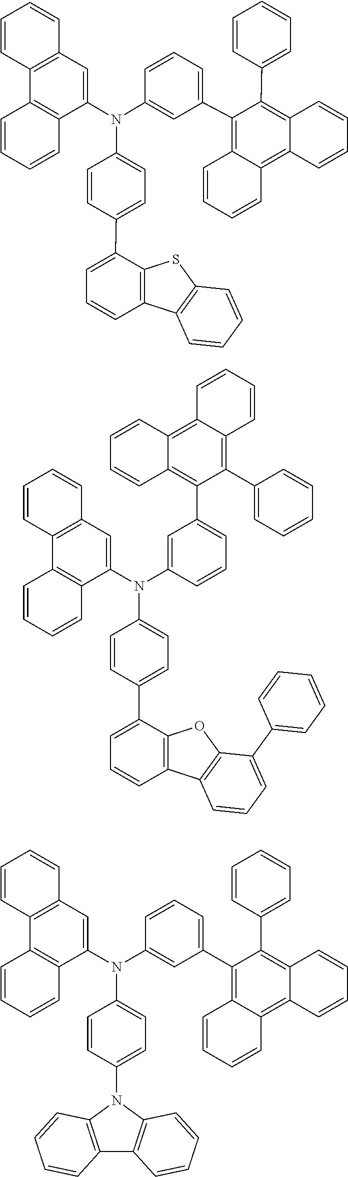 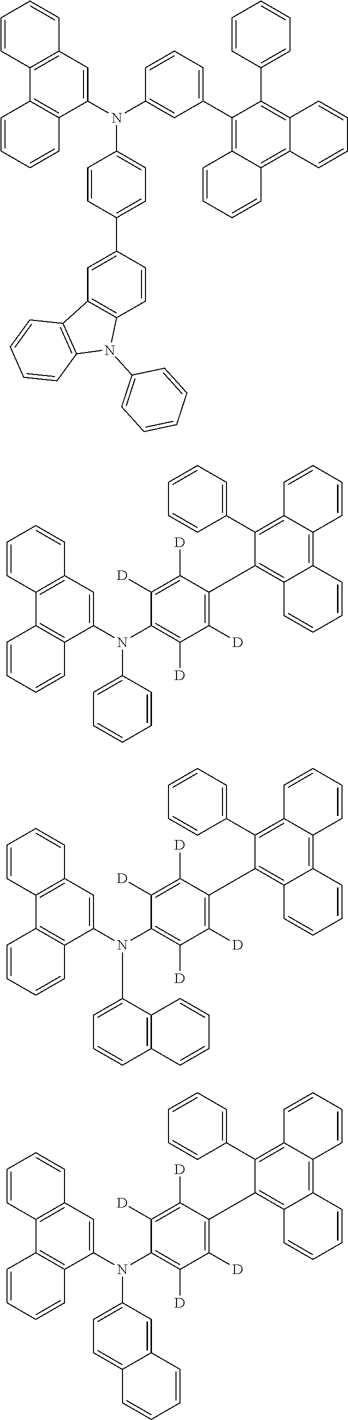 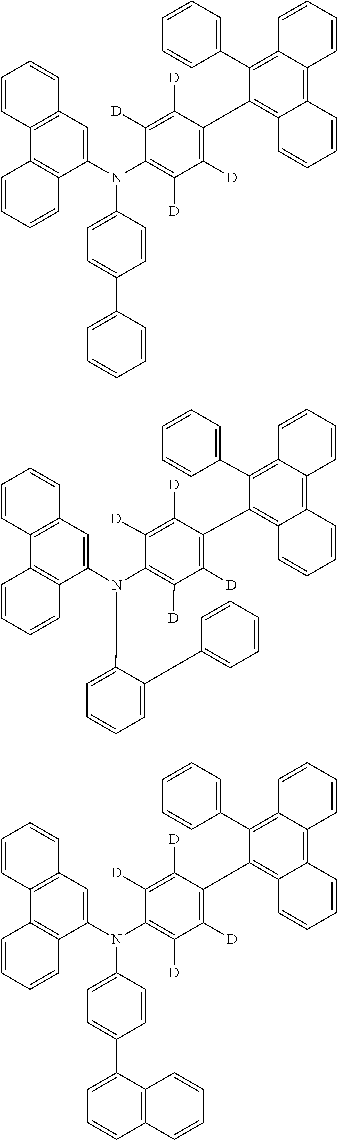 [SMILES: [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12] 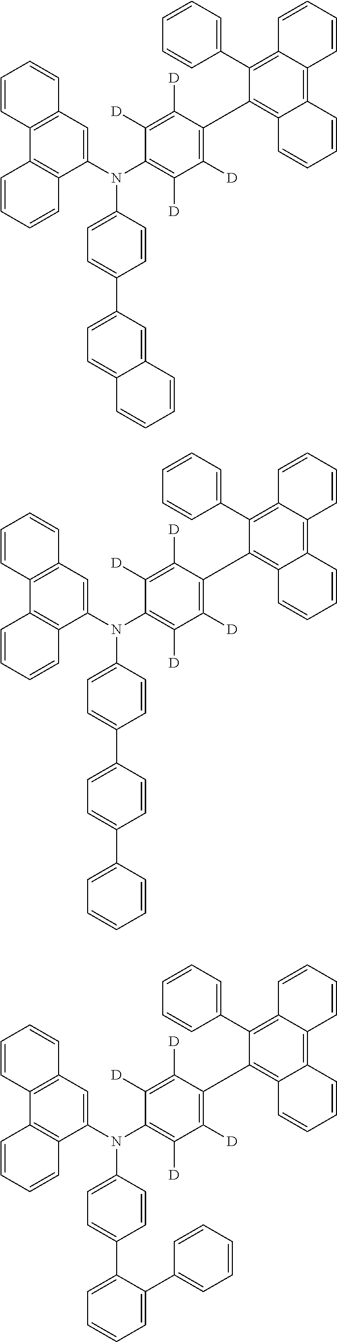 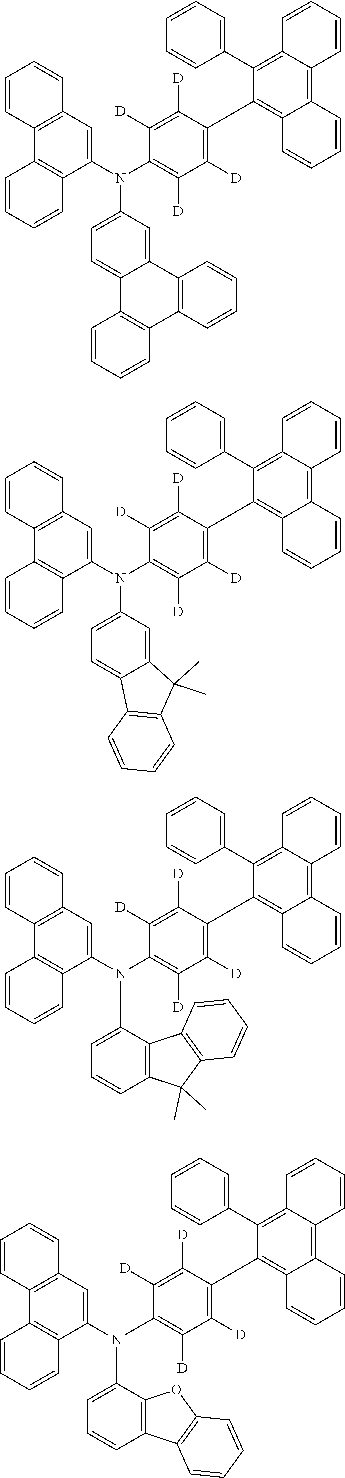 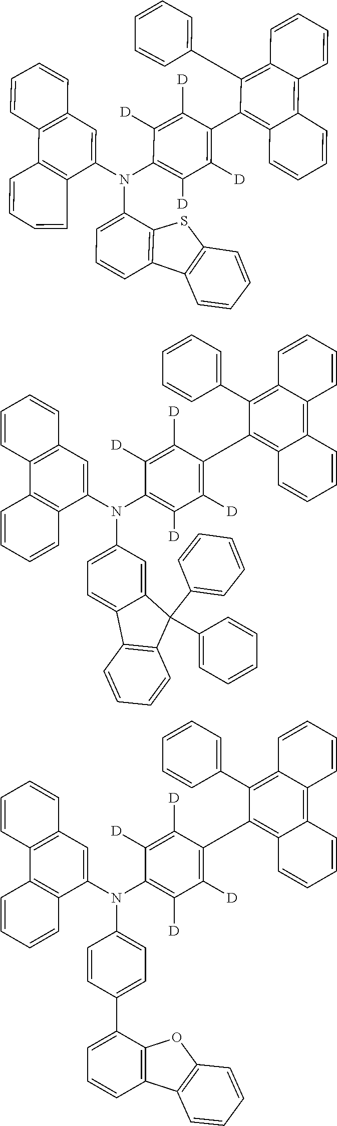 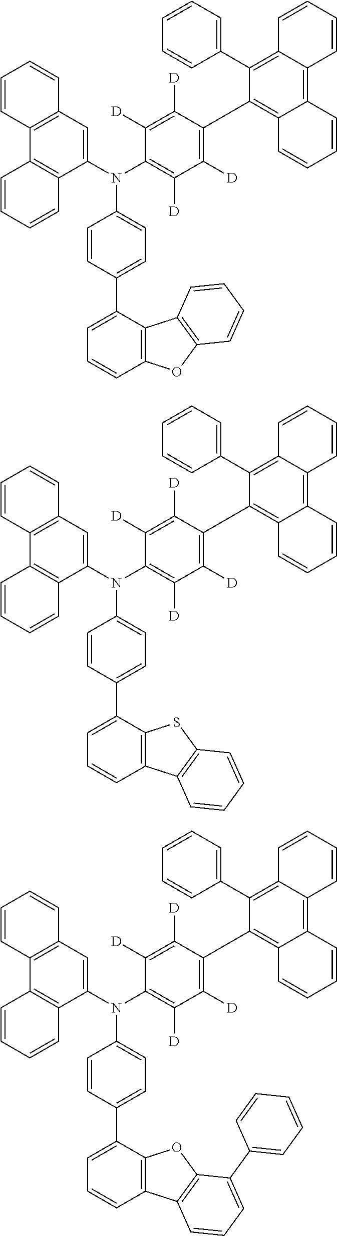 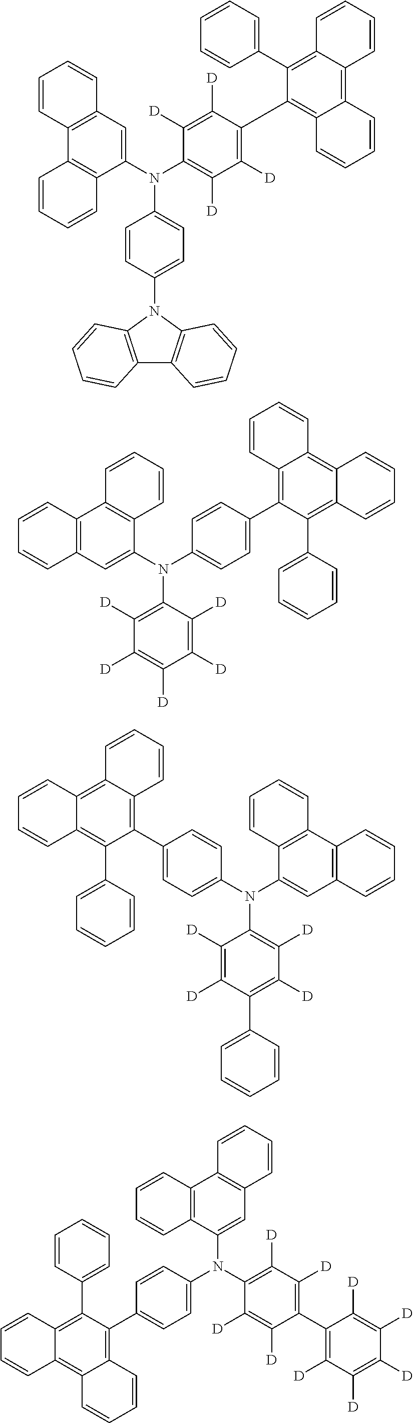 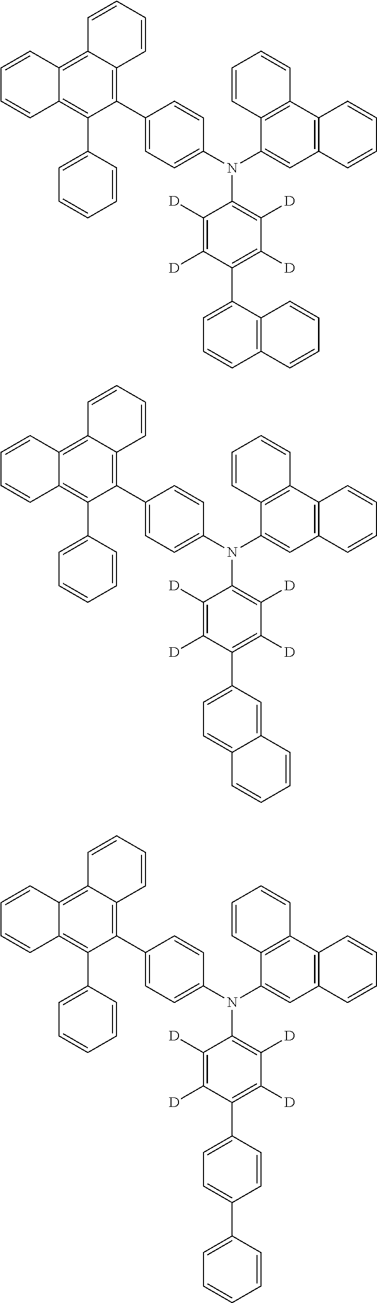 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1] 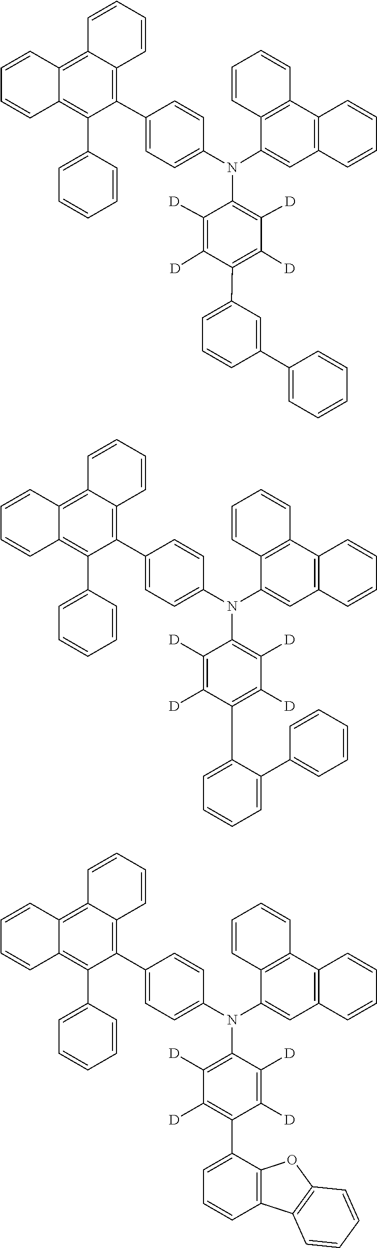 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12] 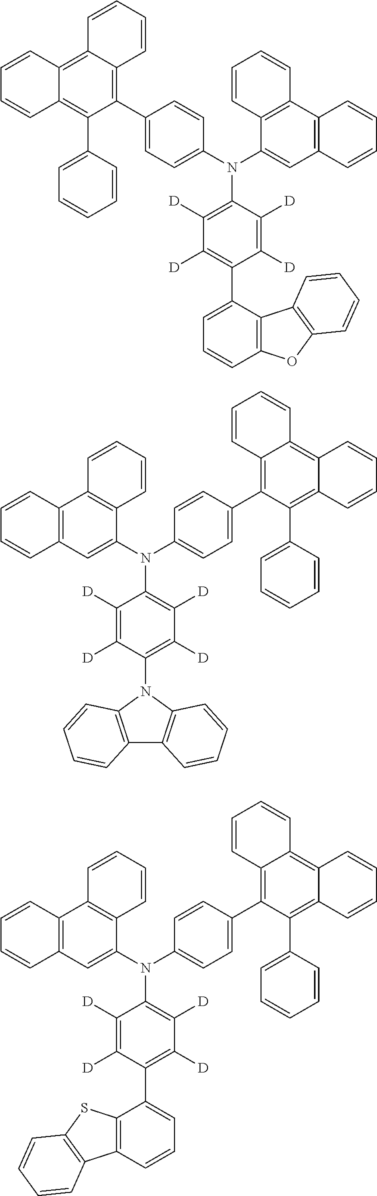 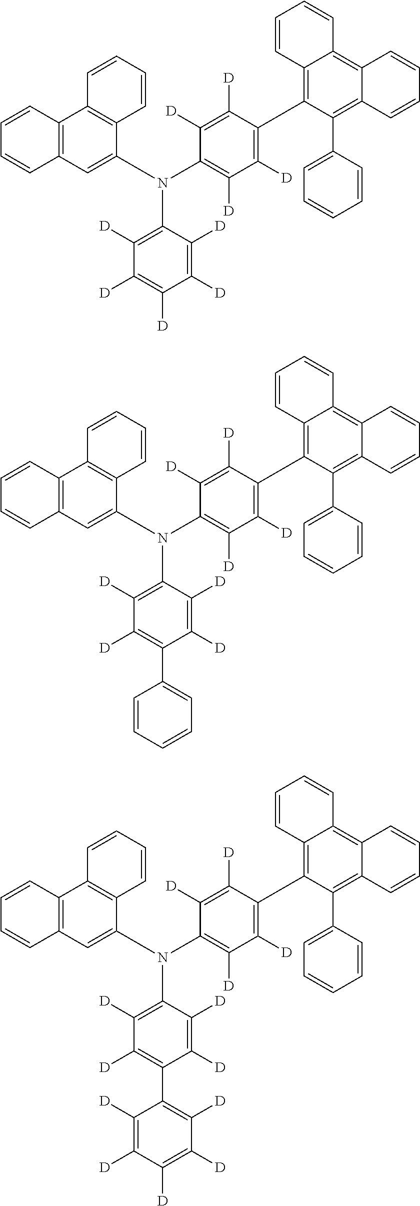 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H]] 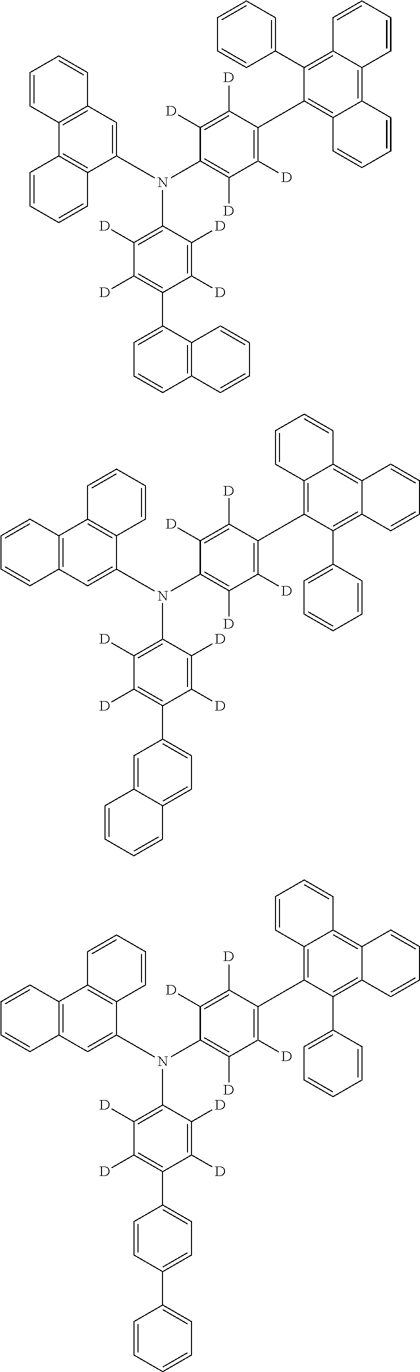 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1] 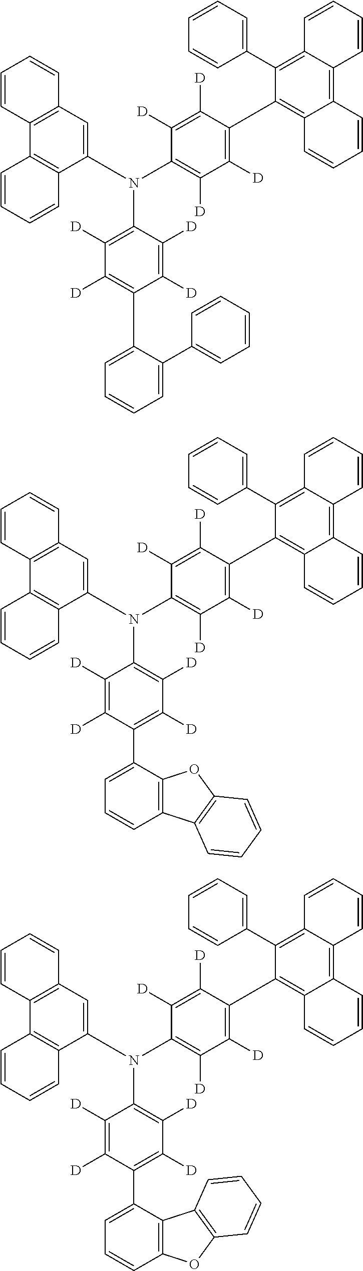 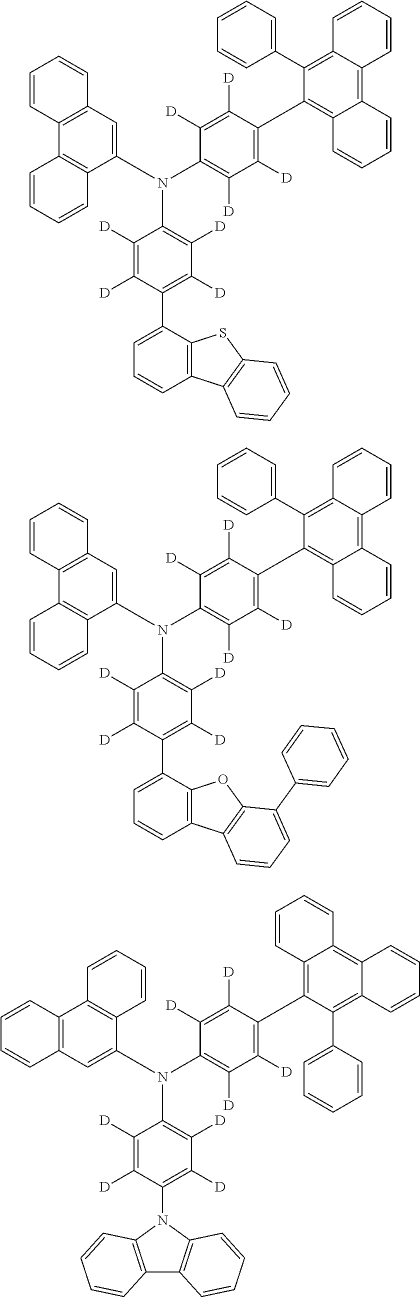 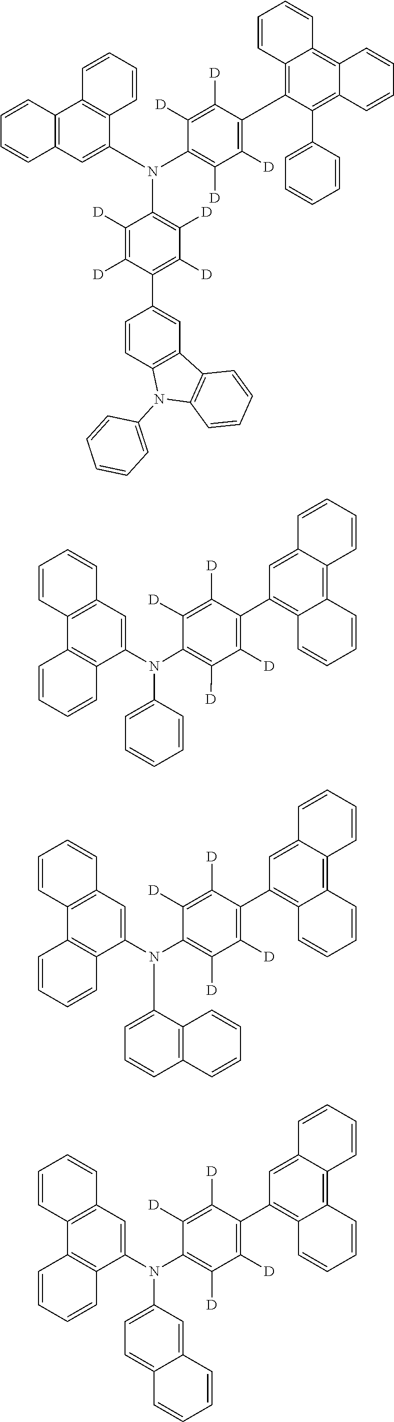 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2c(c1)c1ccccc1n2-c1ccccc1 ; [2H]c1c([2H])c(N(c2ccccc2)c2cc3ccccc3c3ccccc23)c([2H])c([2H])c1-c1cc2ccccc2c2ccccc12 ; [2H]c1c([2H])c(N(c2cccc3ccccc23)c2cc3ccccc3c3ccccc23)c([2H])c([2H])c1-c1cc2ccccc2c2ccccc12 ; [2H]c1c([2H])c(N(c2ccc3ccccc3c2)c2cc3ccccc3c3ccccc23)c([2H])c([2H])c1-c1cc2ccccc2c2ccccc12] 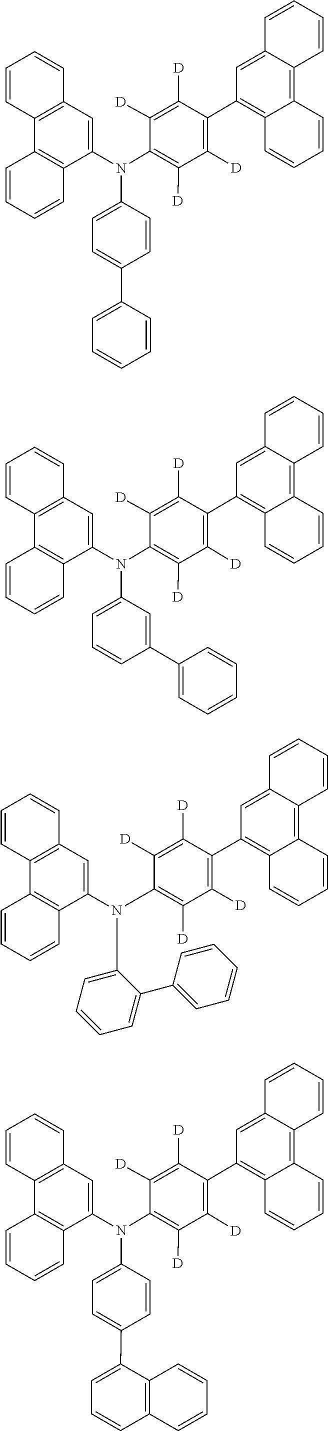 [SMILES: [2H]c1c([2H])c(N(c2ccc(-c3ccccc3)cc2)c2cc3ccccc3c3ccccc23)c([2H])c([2H])c1-c1cc2ccccc2c2ccccc12 ; [2H]c1c([2H])c(N(c2cccc(-c3ccccc3)c2)c2cc3ccccc3c3ccccc23)c([2H])c([2H])c1-c1cc2ccccc2c2ccccc12 ; [2H]c1c([2H])c(N(c2ccccc2-c2ccccc2)c2cc3ccccc3c3ccccc23)c([2H])c([2H])c1-c1cc2ccccc2c2ccccc12 ; [2H]c1c([2H])c(N(c2ccc(-c3cccc4ccccc34)cc2)c2cc3ccccc3c3ccccc23)c([2H])c([2H])c1-c1cc2ccccc2c2ccccc12] 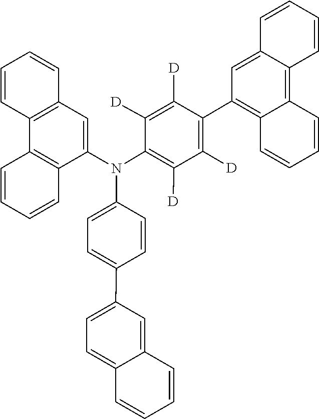 [SMILES: [2H]c1c([2H])c(N(c2ccc(-c3ccc4ccccc4c3)cc2)c2cc3ccccc3c3ccccc23)c([2H])c([2H])c1-c1cc2ccccc2c2ccccc12] 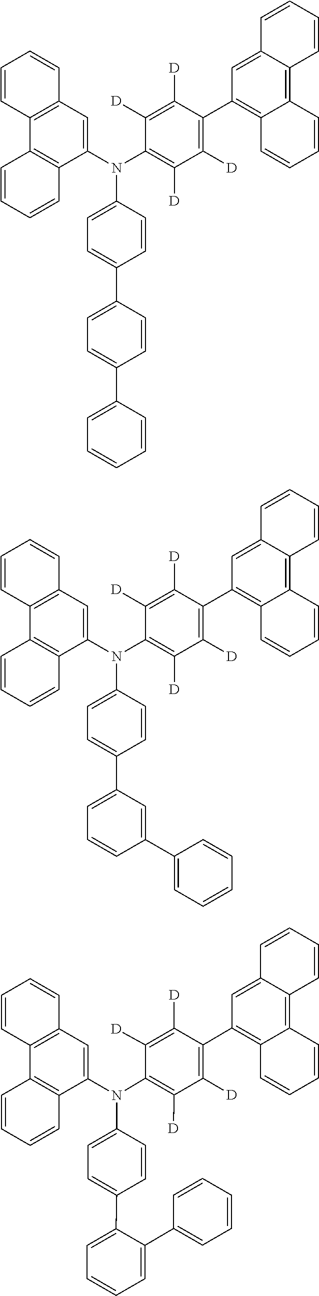 [SMILES: [2H]c1c([2H])c(N(c2ccc(-c3ccc(-c4ccccc4)cc3)cc2)c2cc3ccccc3c3ccccc23)c([2H])c([2H])c1-c1cc2ccccc2c2ccccc12 ; [2H]c1c([2H])c(N(c2ccc(-c3cccc(-c4ccccc4)c3)cc2)c2cc3ccccc3c3ccccc23)c([2H])c([2H])c1-c1cc2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1cc2ccccc2c2ccccc12] 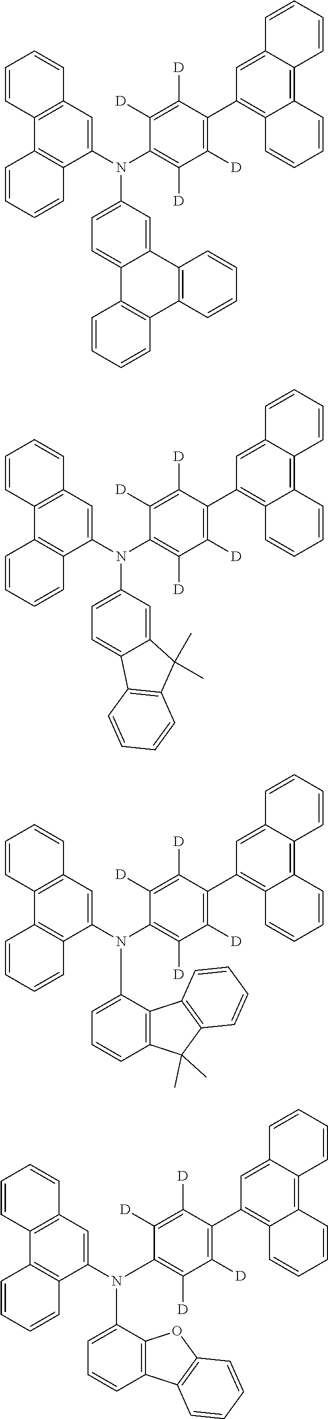 [SMILES: [2H]c1c([2H])c(N(c2ccc3c4ccccc4c4ccccc4c3c2)c2cc3ccccc3c3ccccc23)c([2H])c([2H])c1-c1cc2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(C)C)c2cc3ccccc3c3ccccc23)c([2H])c([2H])c1-c1cc2ccccc2c2ccccc12 ; [2H]c1c([2H])c(N(c2cc3ccccc3c3ccccc23)c2cccc3c2oc2ccccc23)c([2H])c([2H])c1-c1cc2ccccc2c2ccccc12] 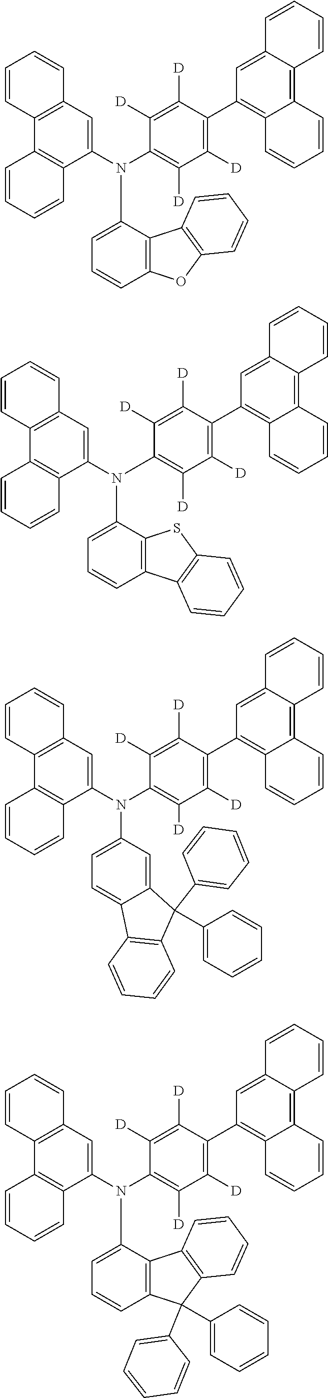 [SMILES: [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12 ; [2H]c1c([2H])c(N(c2ccc3c(c2)C(c2ccccc2)(c2ccccc2)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(c2ccccc2)c2ccccc2)c2cc3ccccc3c3ccccc23)c([2H])c([2H])c1-c1cc2ccccc2c2ccccc12] 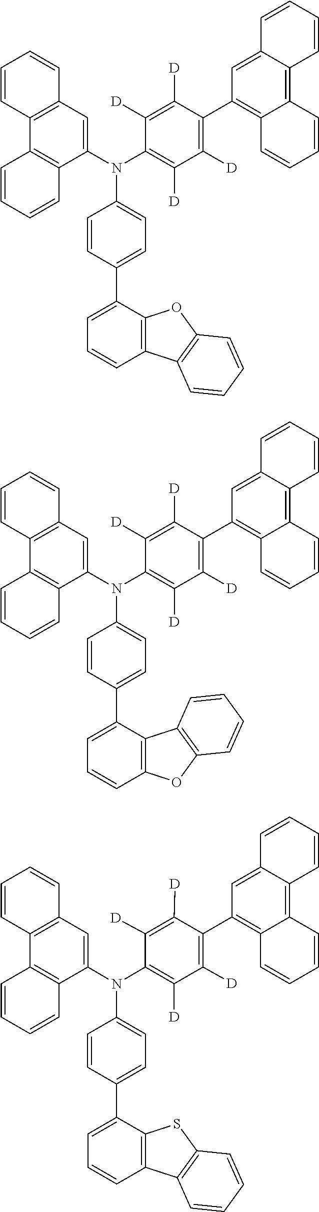 [SMILES: [2H]c1c([2H])c(N(c2ccc(-c3cccc4c3oc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1cc2ccccc2c2ccccc12 ; [2H]c1c([2H])c(N(c2ccc(-c3cccc4c3sc3ccccc34)cc2)c2cc3ccccc3c3ccccc23)c([2H])c([2H])c1-c1cc2ccccc2c2ccccc12] 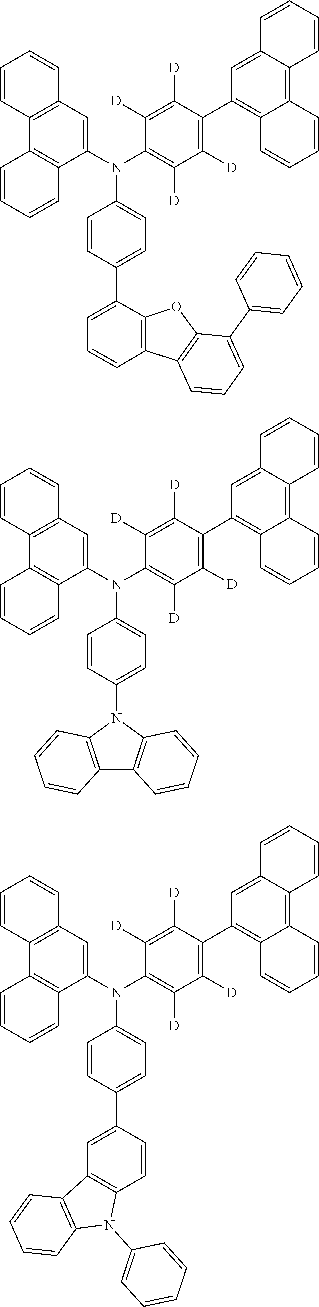 [SMILES: [2H]c1c([2H])c(N(c2ccc(-c3cccc4c3oc3c(-c5ccccc5)cccc34)cc2)c2cc3ccccc3c3ccccc23)c([2H])c([2H])c1-c1cc2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1cc2ccccc2c2ccccc12 ; [2H]c1c([2H])c(N(c2ccc(-c3ccc4c(c3)c3ccccc3n4-c3ccccc3)cc2)c2cc3ccccc3c3ccccc23)c([2H])c([2H])c1-c1cc2ccccc2c2ccccc12] 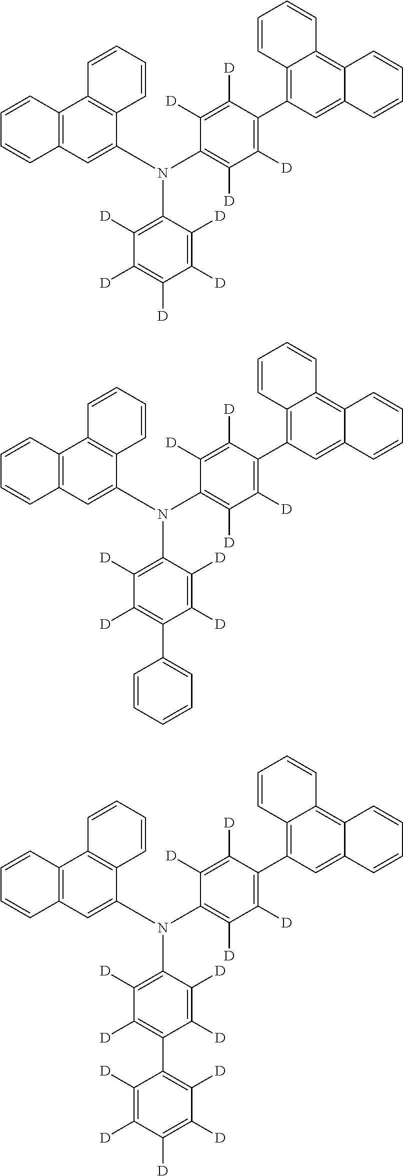 [SMILES: [2H]c1c([2H])c([2H])c(N(c2c([2H])c([2H])c(-c3cc4ccccc4c4ccccc34)c([2H])c2[2H])c2cc3ccccc3c3ccccc23)c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4cc5ccccc5c5ccccc45)c([2H])c3[2H])c3cc4ccccc4c4ccccc34)c([2H])c2[2H])c([2H])c1[2H]] 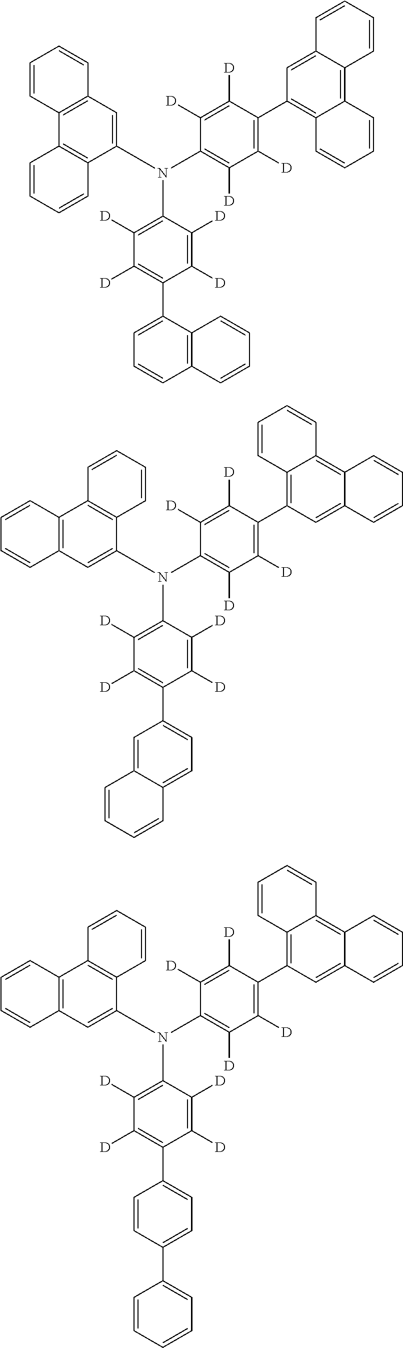 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1] 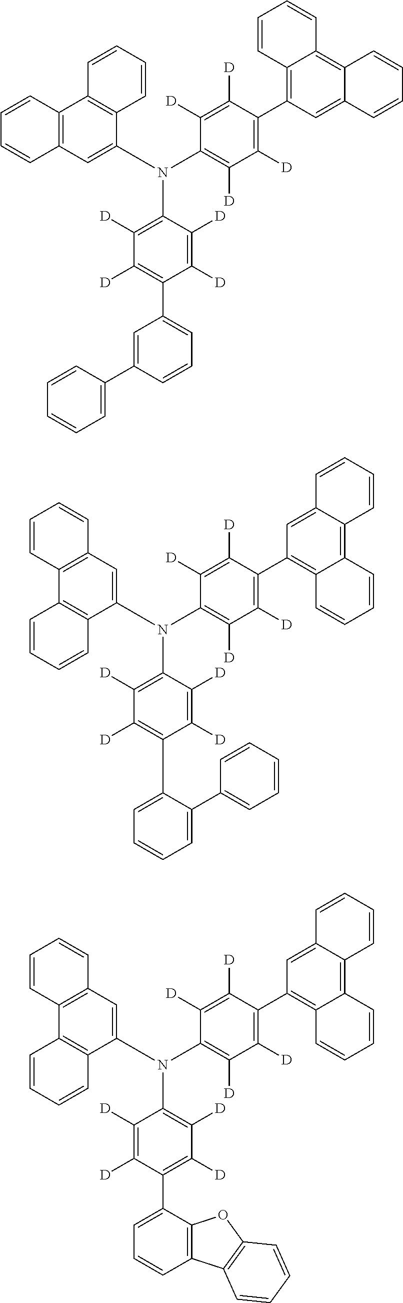 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1cc2ccccc2c2ccccc12] 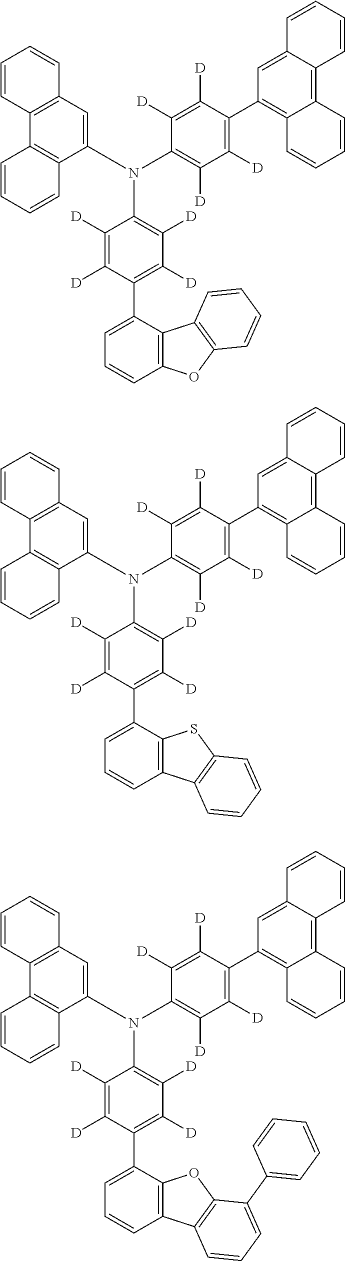 [SMILES: [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1cc2ccccc2c2ccccc12] 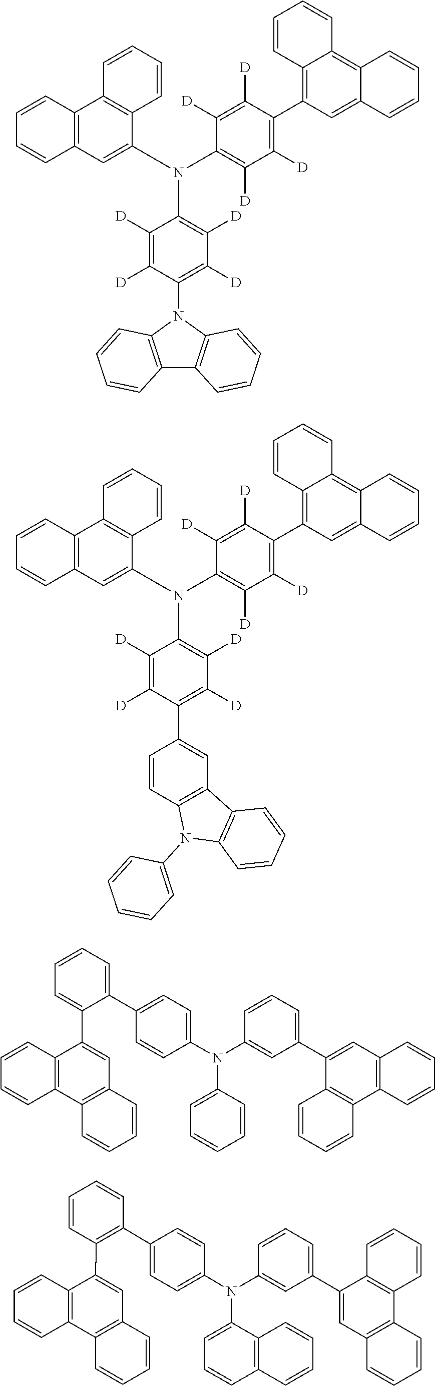 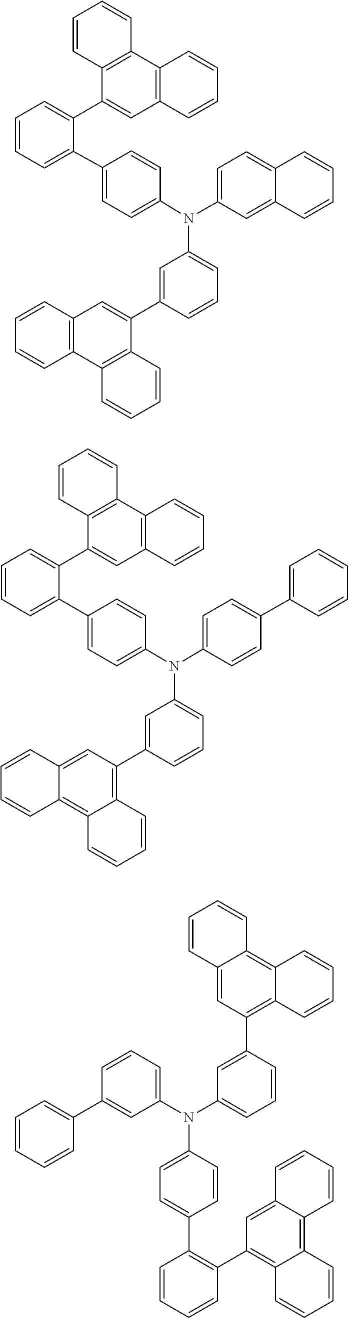 [SMILES: c1cc(-c2cc3ccccc3c3ccccc23)cc(N(c2ccc(-c3ccccc3-c3cc4ccccc4c4ccccc34)cc2)c2ccc3ccccc3c2)c1 ; c1ccc(-c2ccc(N(c3ccc(-c4ccccc4-c4cc5ccccc5c5ccccc45)cc3)c3cccc(-c4cc5ccccc5c5ccccc45)c3)cc2)cc1 ; c1ccc(-c2cccc(N(c3ccc(-c4ccccc4-c4cc5ccccc5c5ccccc45)cc3)c3cccc(-c4cc5ccccc5c5ccccc45)c3)c2)cc1] 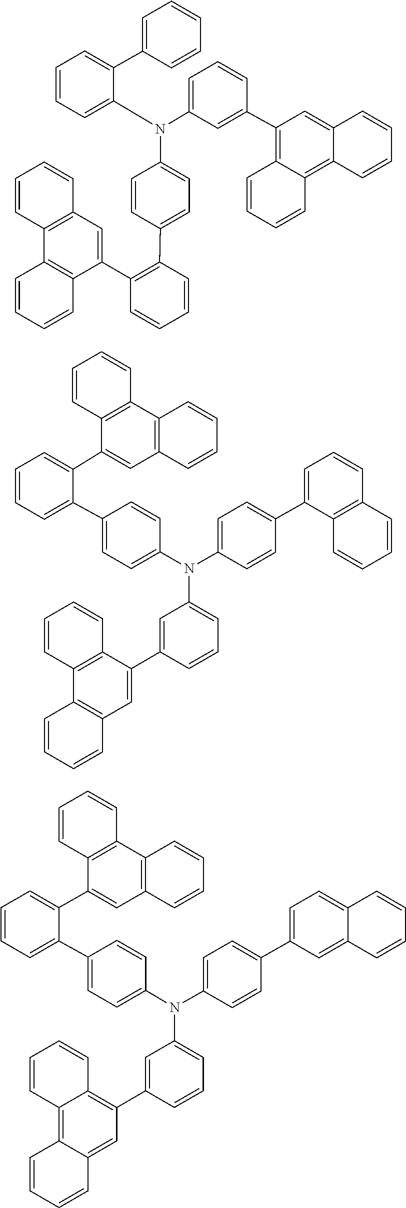 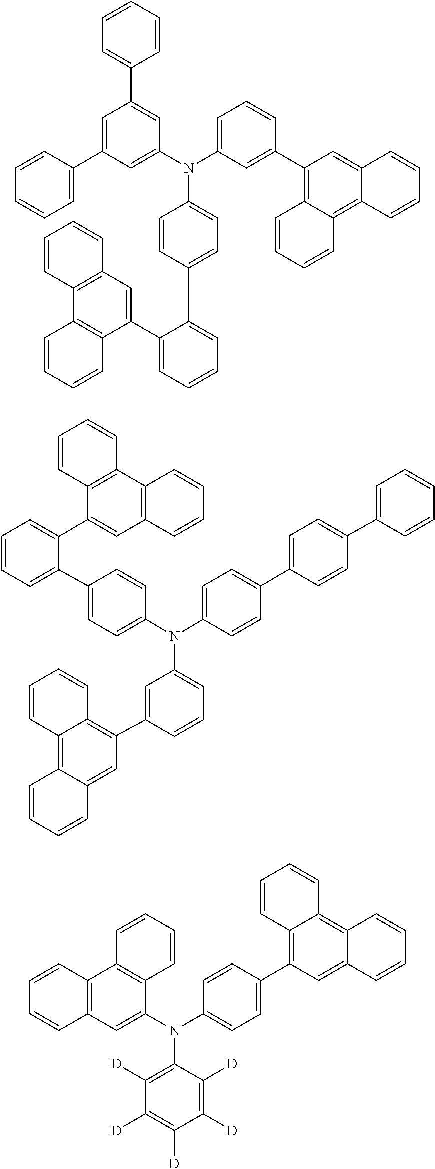 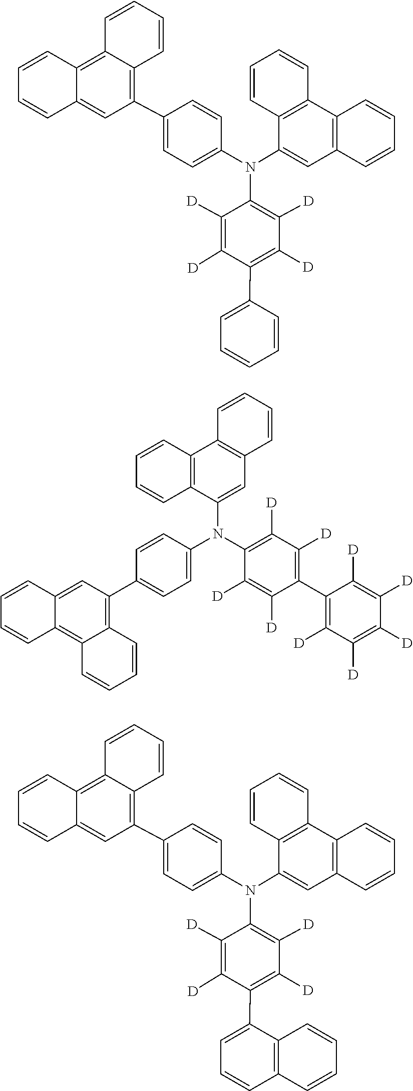 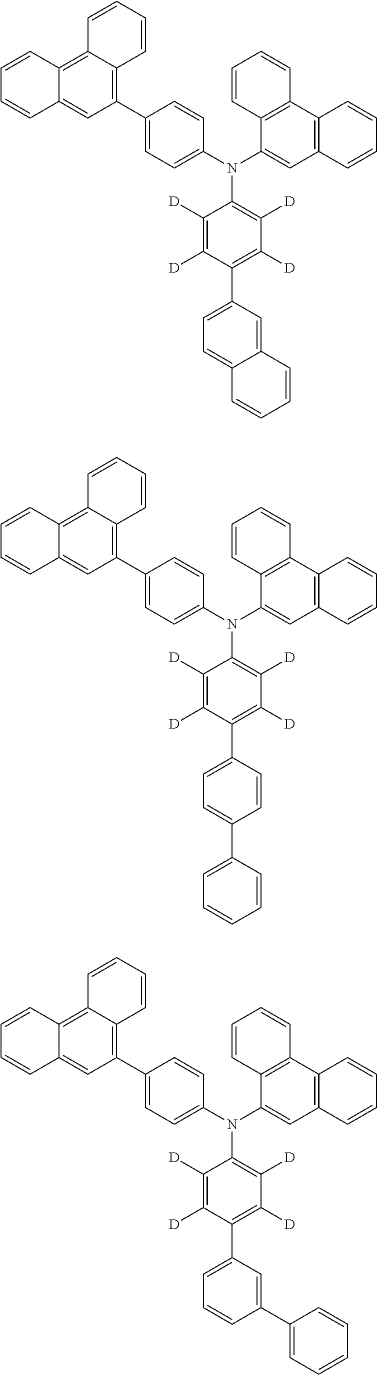 [SMILES: [2H]c1c([2H])c(N(c2ccc(-c3cc4ccccc4c4ccccc34)cc2)c2cc3ccccc3c3ccccc23)c([2H])c([2H])c1-c1ccc2ccccc2c1 ; [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1] 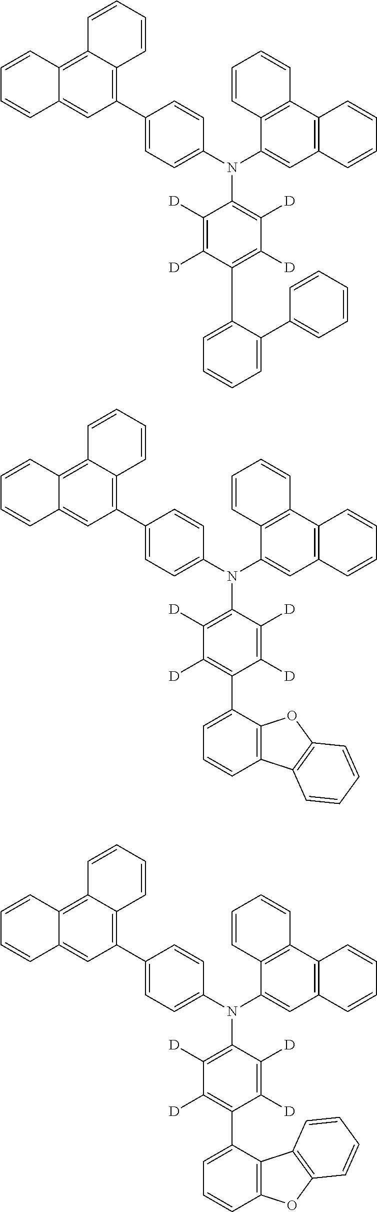 [SMILES: [2H]c1c([2H])c(N(c2ccc(-c3cc4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3cc4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3cc4ccccc4c4ccccc34)cc2)c2cc3ccccc3c3ccccc23)c([2H])c([2H])c1-c1cccc2oc3ccccc3c12] 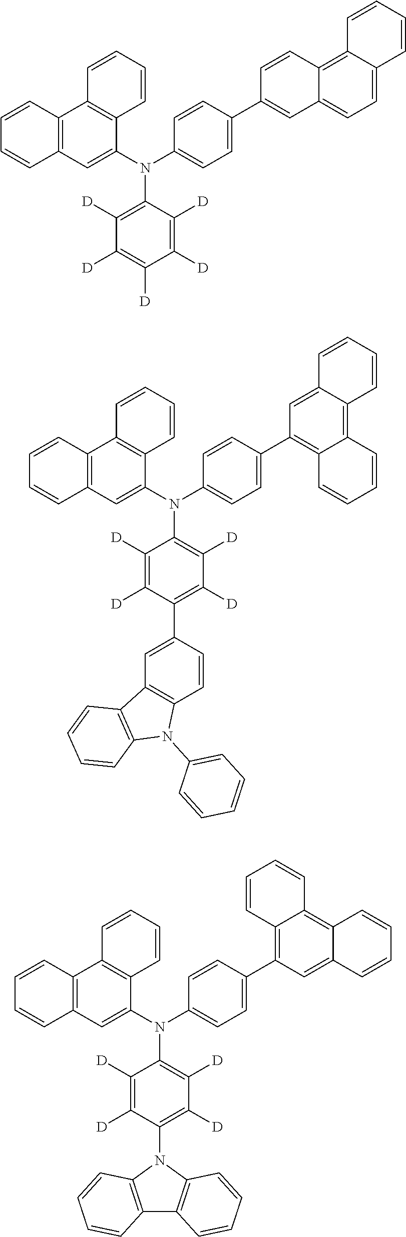 [SMILES: [2H]c1c([2H])c([2H])c(N(c2ccc(-c3ccc4c(ccc5ccccc54)c3)cc2)c2cc3ccccc3c3ccccc23)c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1ccc2c(c1)c1ccccc1n2-c1ccccc1 ; [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12] 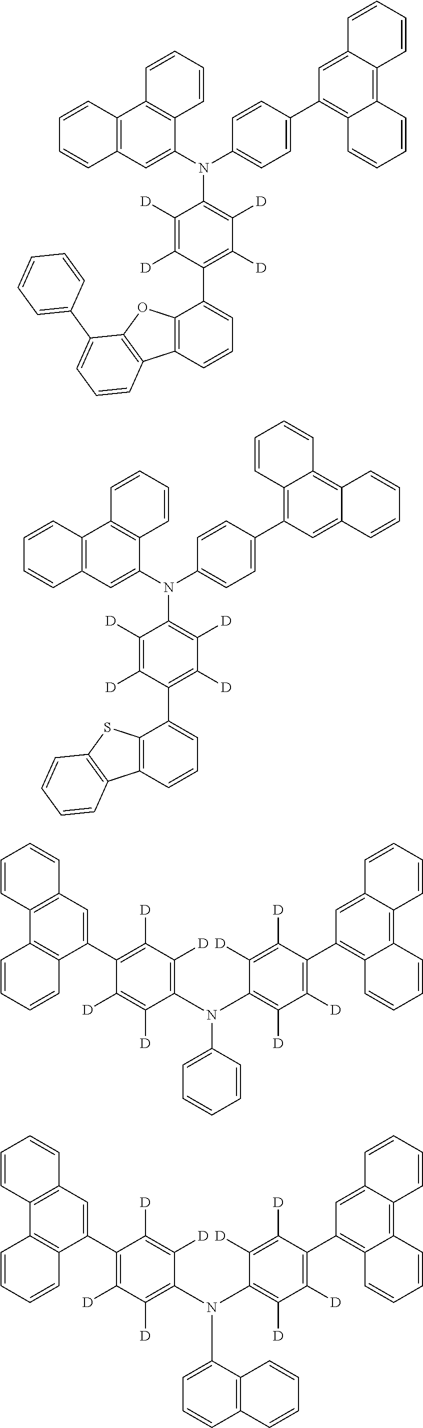 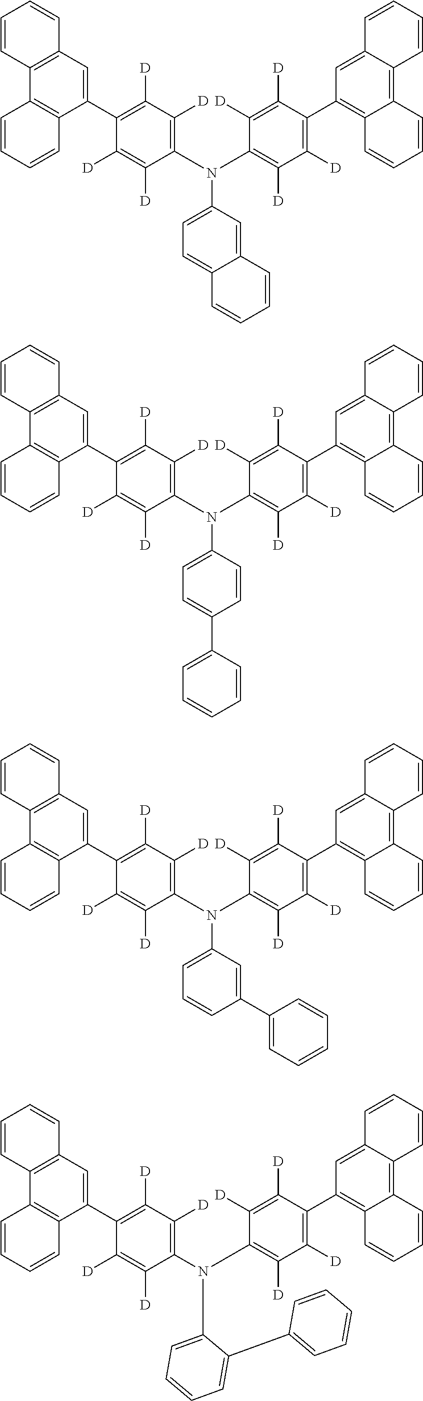 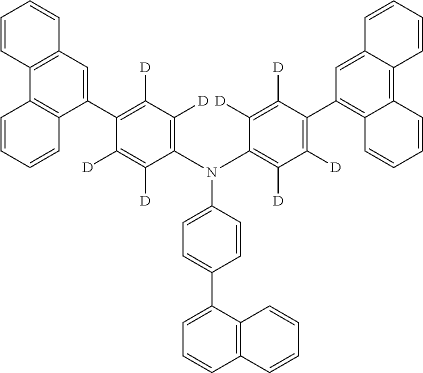 [SMILES: [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12] 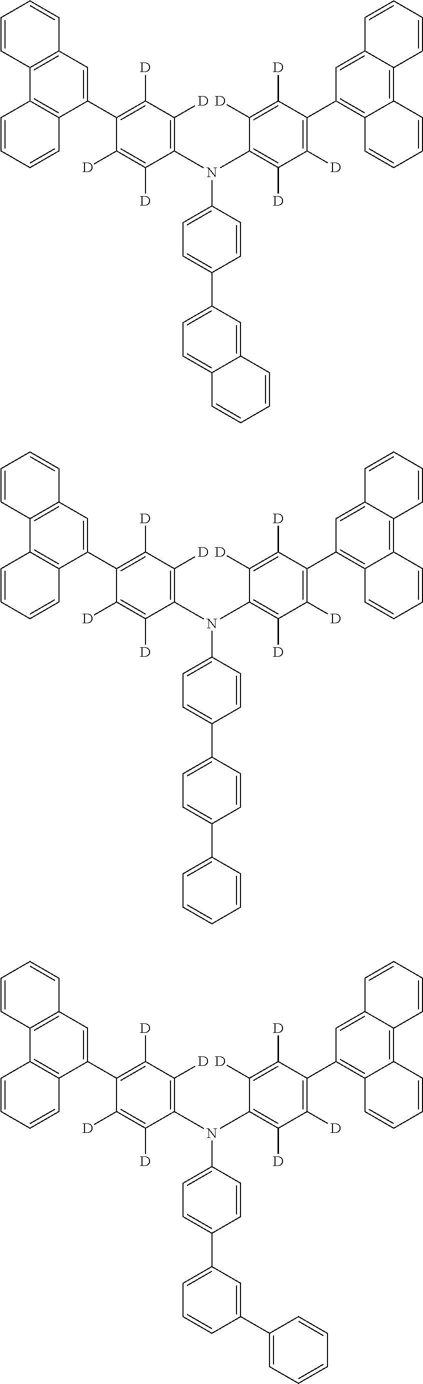 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc(-c4ccccc4)c3)cc2)c2c([2H])c([2H])c(-c3cc4ccccc4c4ccccc34)c([2H])c2[2H])c([2H])c([2H])c1-c1cc2ccccc2c2ccccc12] 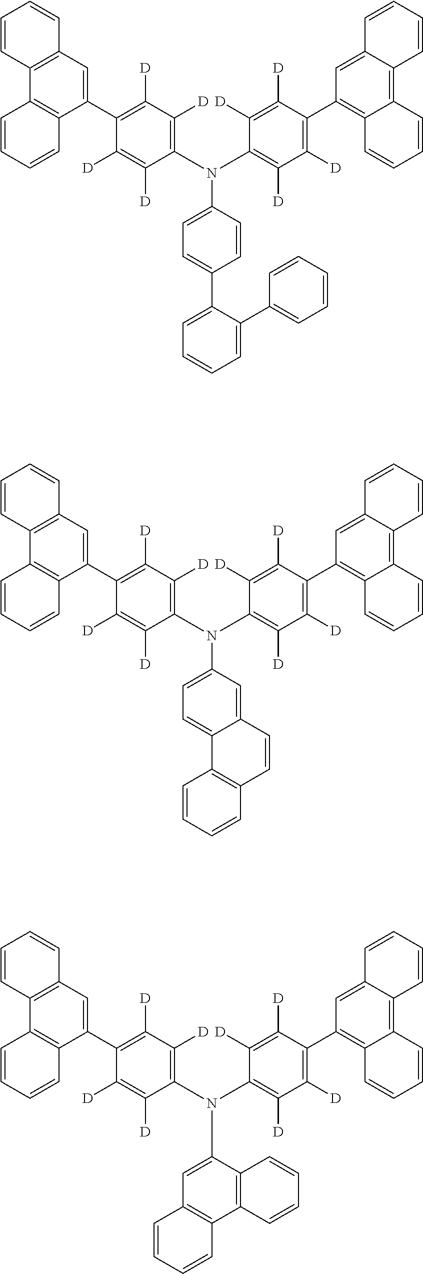 [SMILES: [2H]c1c([2H])c(N(c2ccc(-c3ccccc3-c3ccccc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c(ccc4ccccc43)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cc2ccccc2c2ccccc12] 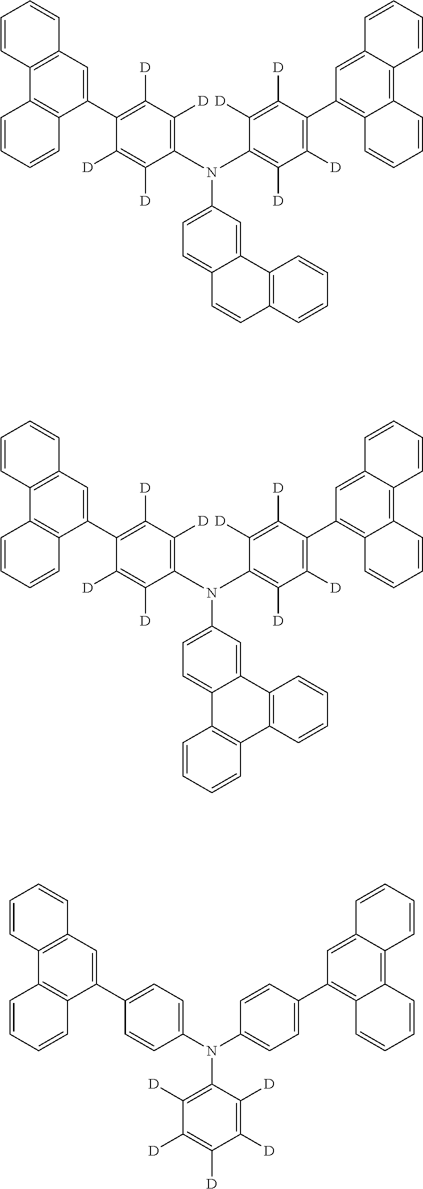 [SMILES: [2H]c1c([2H])c(N(c2ccc3ccc4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c4ccccc4c4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c([2H])c(N(c2ccc(-c3cc4ccccc4c4ccccc34)cc2)c2ccc(-c3cc4ccccc4c4ccccc34)cc2)c([2H])c1[2H]] 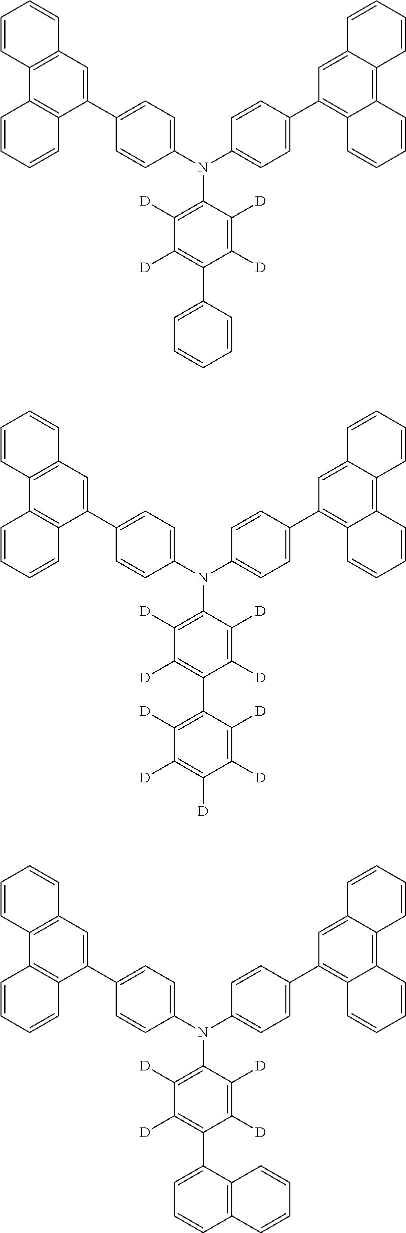 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3ccc(-c4cc5ccccc5c5ccccc45)cc3)c([2H])c2[2H])c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1cccc2ccccc12] 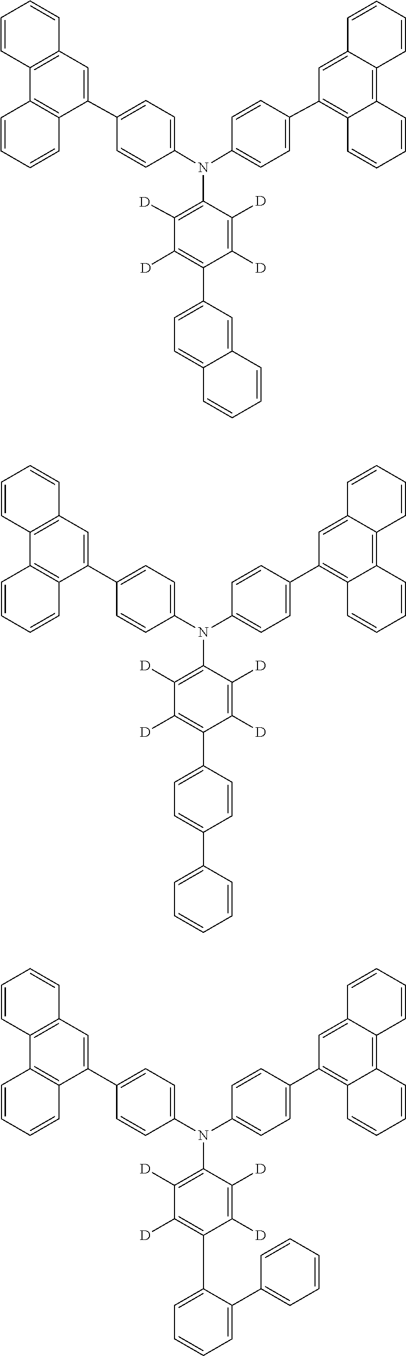 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc2ccccc2c1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc(-c2ccccc2)cc1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1-c1ccccc1] 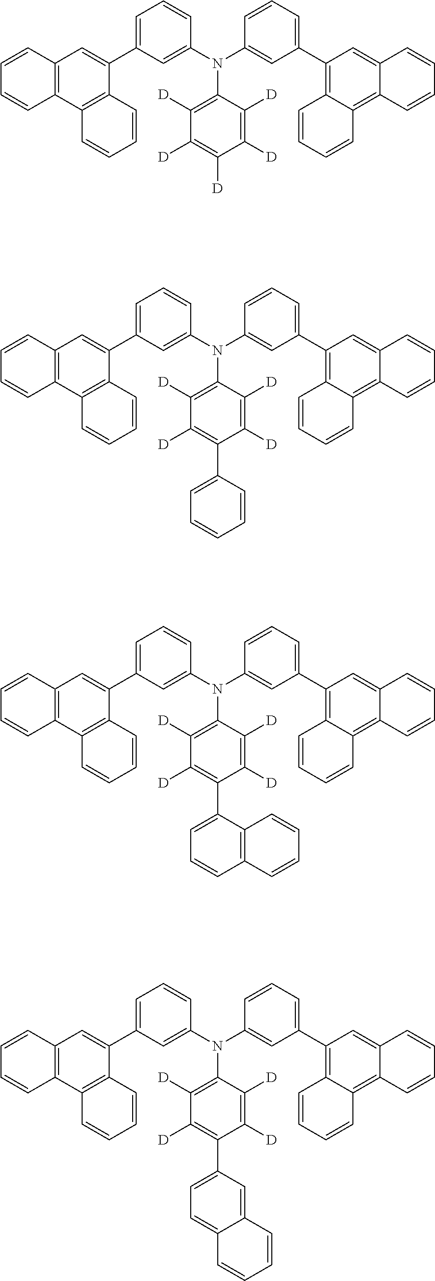 [SMILES: [2H]c1c([2H])c([2H])c(N(c2cccc(-c3cc4ccccc4c4ccccc34)c2)c2cccc(-c3cc4ccccc4c4ccccc34)c2)c([2H])c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1cccc2ccccc12 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccc2ccccc2c1] 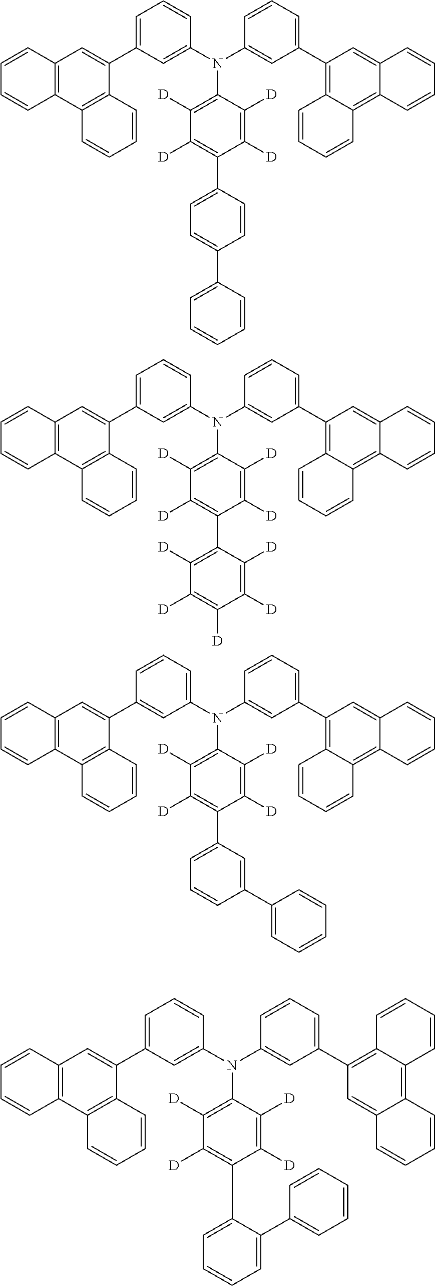 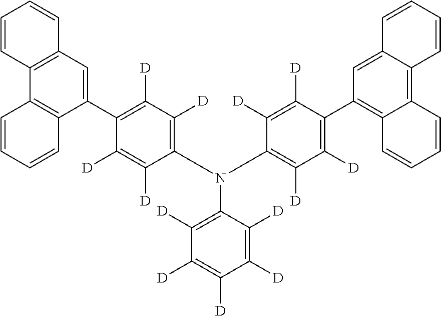 [SMILES: [2H]c1c([2H])c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c1[2H]] 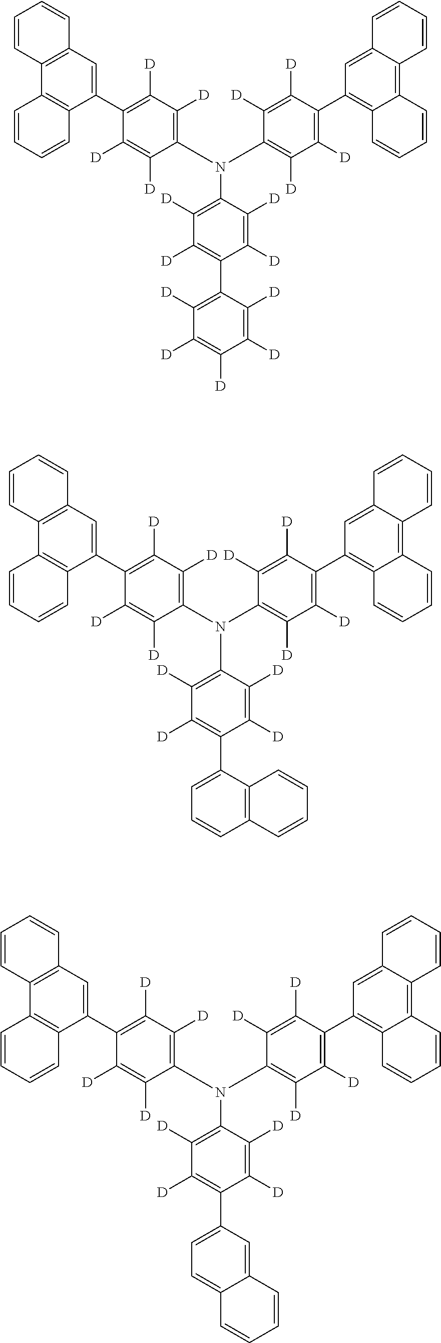 [SMILES: [2H]c1c([2H])c([2H])c(-c2c([2H])c([2H])c(N(c3c([2H])c([2H])c(-c4cc5ccccc5c5ccccc45)c([2H])c3[2H])c3c([2H])c([2H])c(-c4cc5ccccc5c5ccccc45)c([2H])c3[2H])c([2H])c2[2H])c([2H])c1[2H] ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1cccc2ccccc12 ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccc2ccccc2c1] 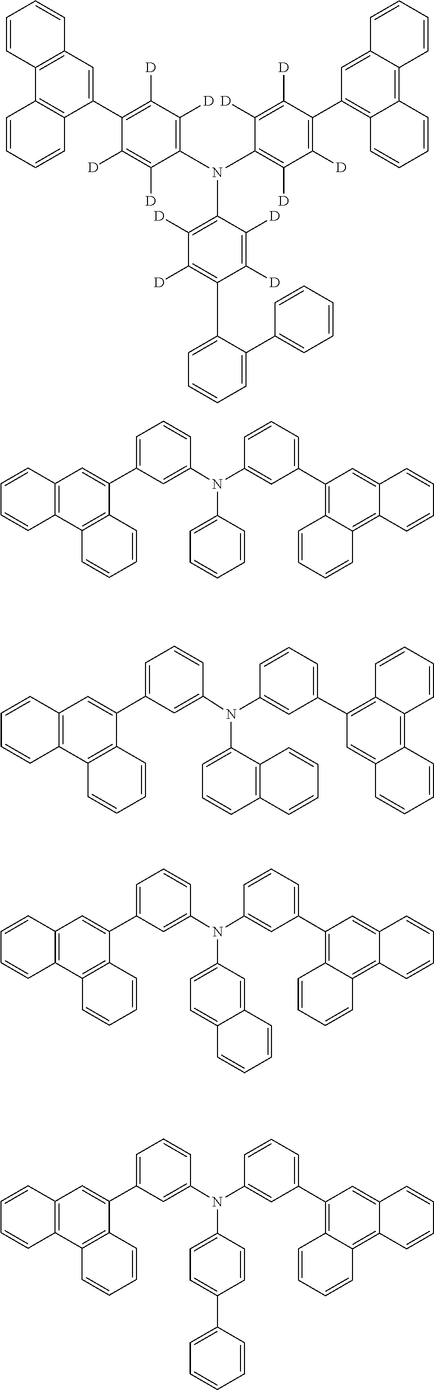 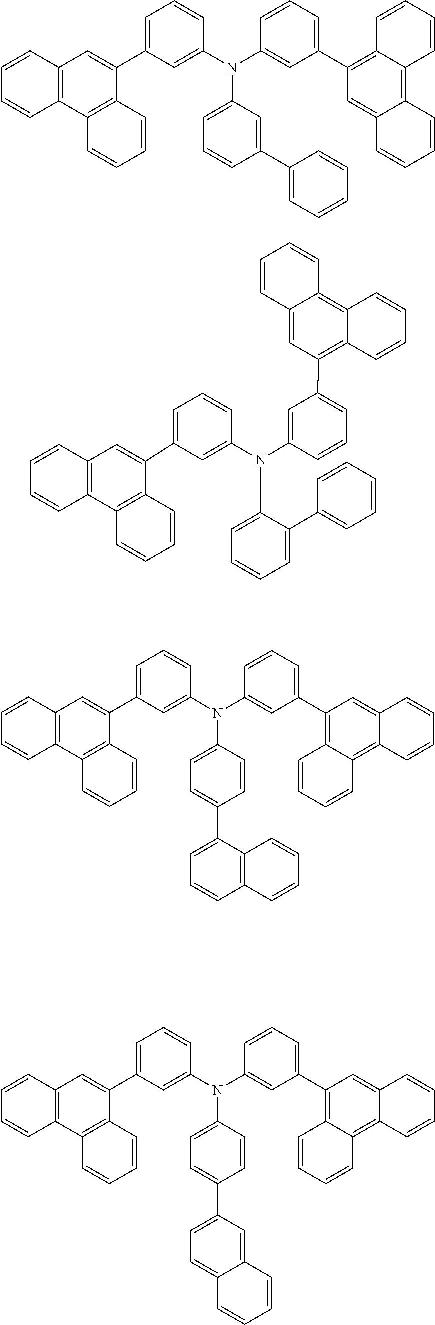 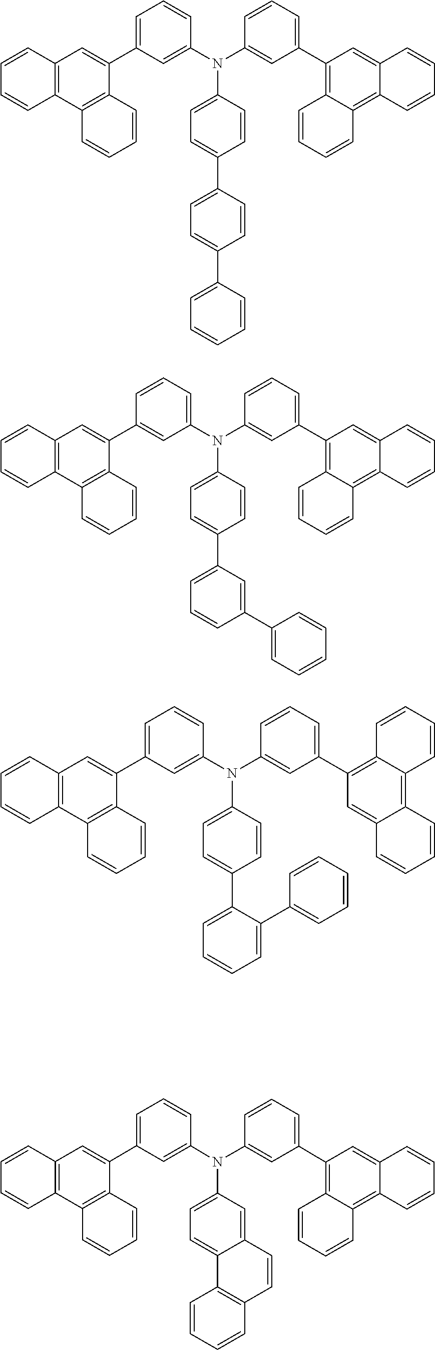 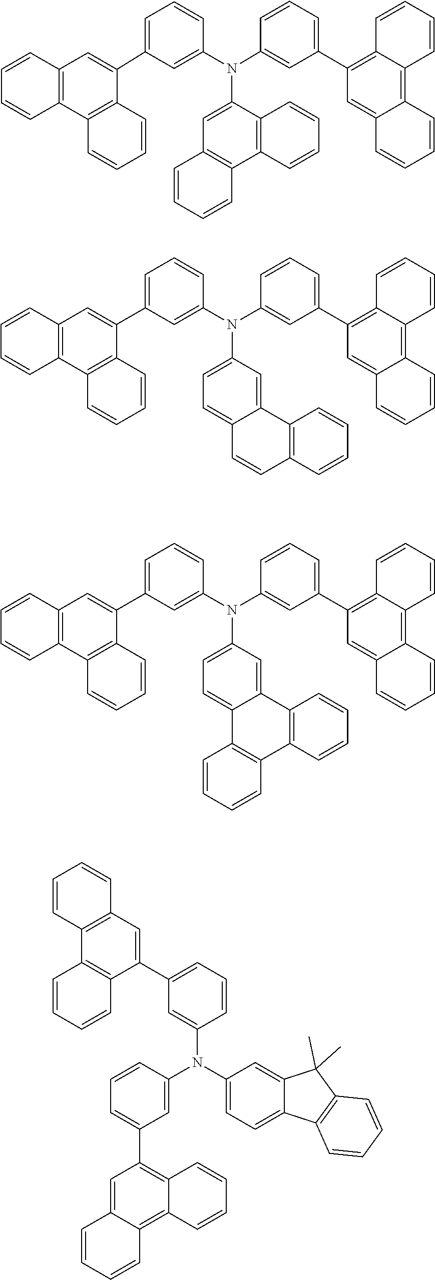 [SMILES: c1cc(-c2cc3ccccc3c3ccccc23)cc(N(c2cccc(-c3cc4ccccc4c4ccccc34)c2)c2cc3ccccc3c3ccccc23)c1 ; c1cc(-c2cc3ccccc3c3ccccc23)cc(N(c2cccc(-c3cc4ccccc4c4ccccc34)c2)c2ccc3ccc4ccccc4c3c2)c1 ; c1cc(-c2cc3ccccc3c3ccccc23)cc(N(c2cccc(-c3cc4ccccc4c4ccccc34)c2)c2ccc3c4ccccc4c4ccccc4c3c2)c1 ; CC1(C)c2ccccc2-c2ccc(N(c3cccc(-c4cc5ccccc5c5ccccc45)c3)c3cccc(-c4cc5ccccc5c5ccccc45)c3)cc21] 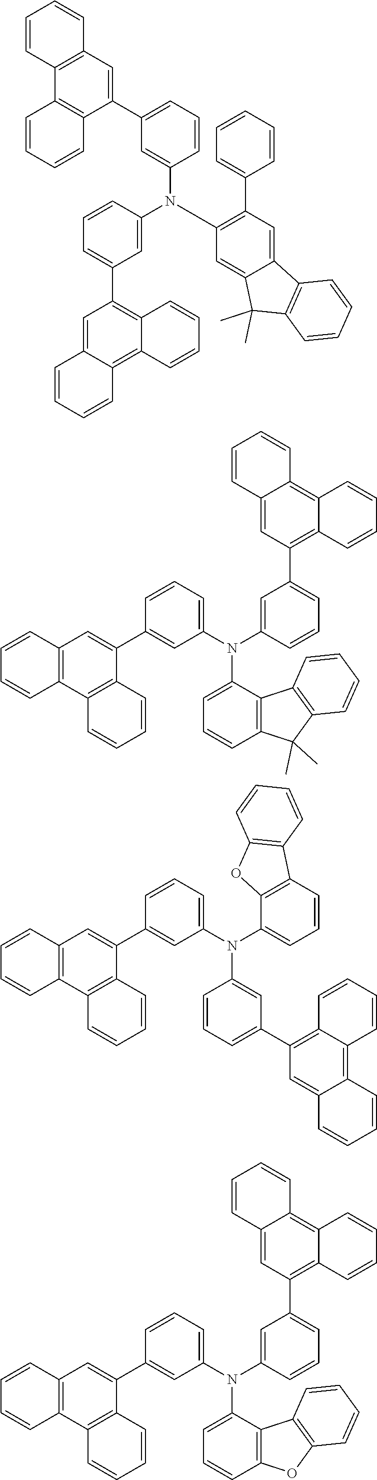 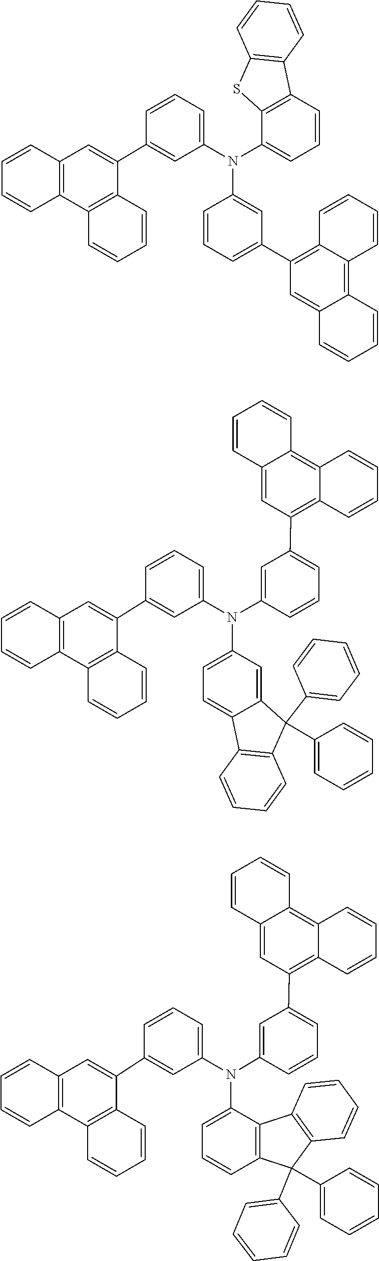 [SMILES: c1cc(-c2cc3ccccc3c3ccccc23)cc(N(c2cccc(-c3cc4ccccc4c4ccccc34)c2)c2cccc3c2sc2ccccc23)c1 ; c1ccc(C2(c3ccccc3)c3ccccc3-c3ccc(N(c4cccc(-c5cc6ccccc6c6ccccc56)c4)c4cccc(-c5cc6ccccc6c6ccccc56)c4)cc32)cc1 ; c1ccc(C2(c3ccccc3)c3ccccc3-c3c(N(c4cccc(-c5cc6ccccc6c6ccccc56)c4)c4cccc(-c5cc6ccccc6c6ccccc56)c4)cccc32)cc1] 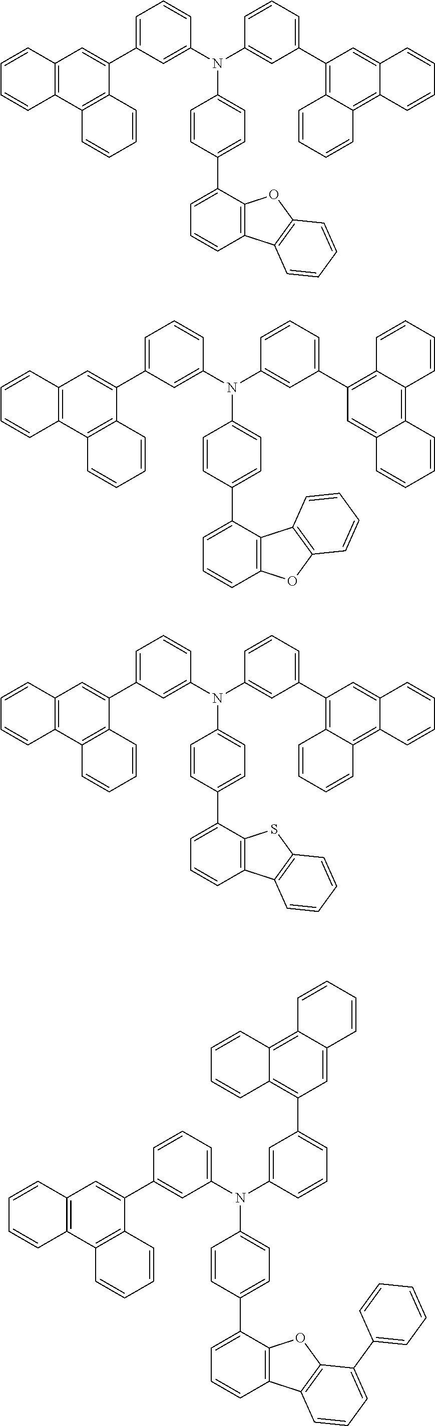 [SMILES: c1cc(-c2cc3ccccc3c3ccccc23)cc(N(c2ccc(-c3cccc4c3oc3ccccc34)cc2)c2cccc(-c3cc4ccccc4c4ccccc34)c2)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1 ; c1cc(-c2cc3ccccc3c3ccccc23)cc(N(c2ccc(-c3cccc4c3sc3ccccc34)cc2)c2cccc(-c3cc4ccccc4c4ccccc34)c2)c1 ; c1ccc(-c2cccc3c2oc2c(-c4ccc(N(c5cccc(-c6cc7ccccc7c7ccccc67)c5)c5cccc(-c6cc7ccccc7c7ccccc67)c5)cc4)cccc23)cc1] 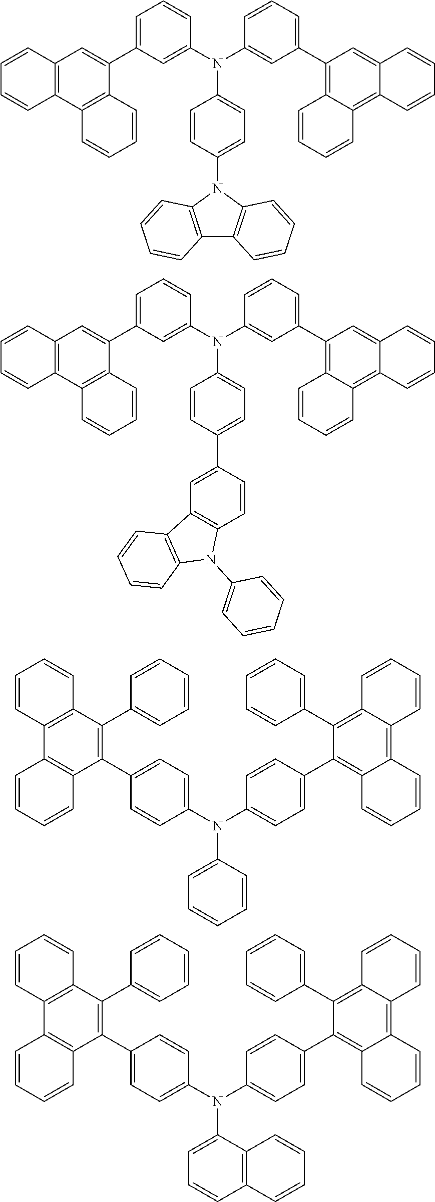 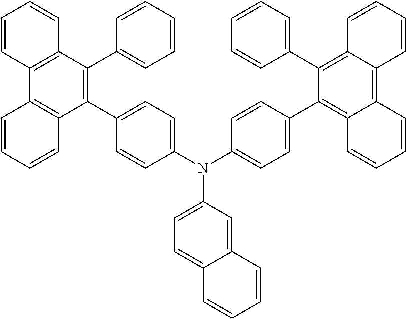 [SMILES: c1ccc(-c2c(-c3ccc(N(c4ccc(-c5c(-c6ccccc6)c6ccccc6c6ccccc56)cc4)c4ccc5ccccc5c4)cc3)c3ccccc3c3ccccc23)cc1] 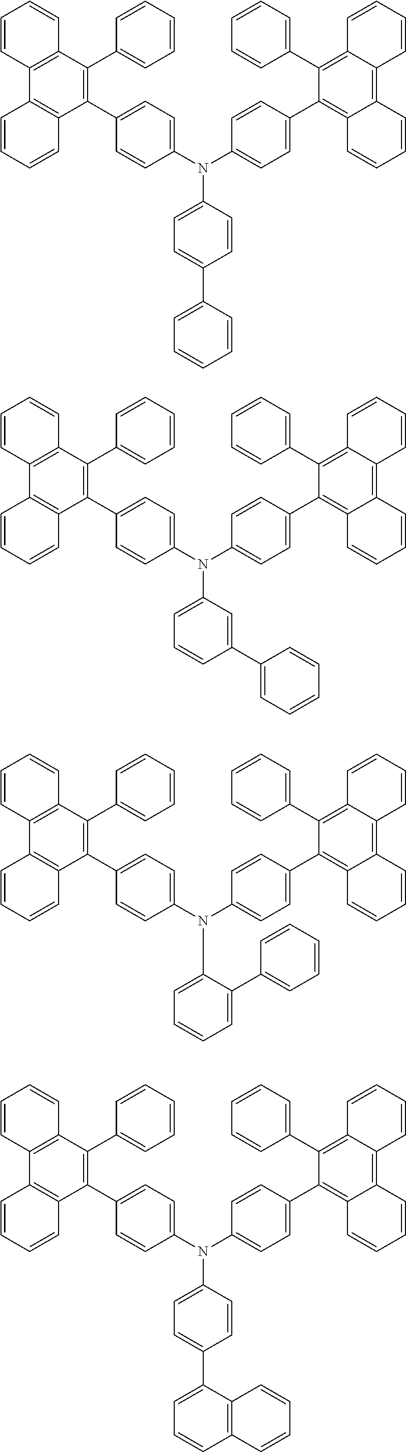 [SMILES: c1ccc(-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1 ; c1ccc(-c2cccc(N(c3ccc(-c4c(-c5ccccc5)c5ccccc5c5ccccc45)cc3)c3ccc(-c4c(-c5ccccc5)c5ccccc5c5ccccc45)cc3)c2)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1] 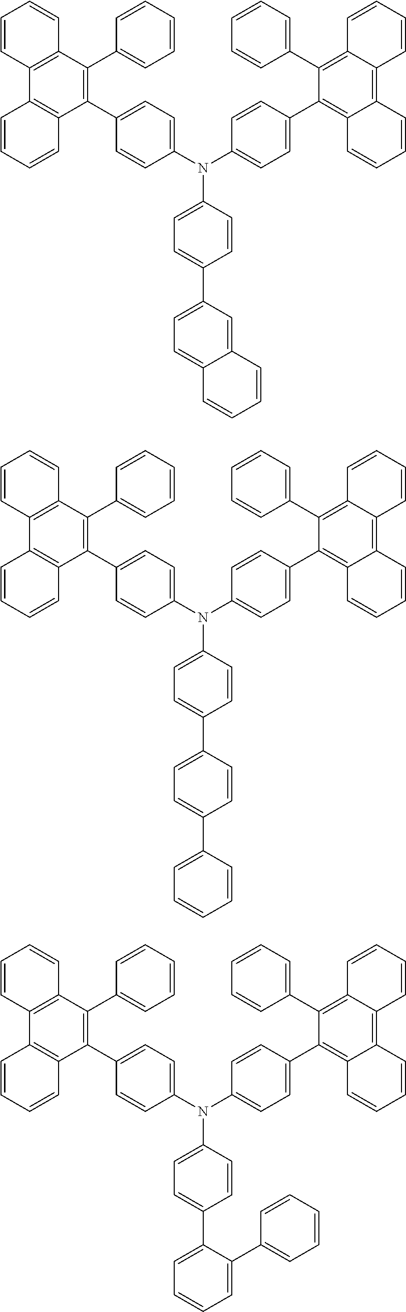 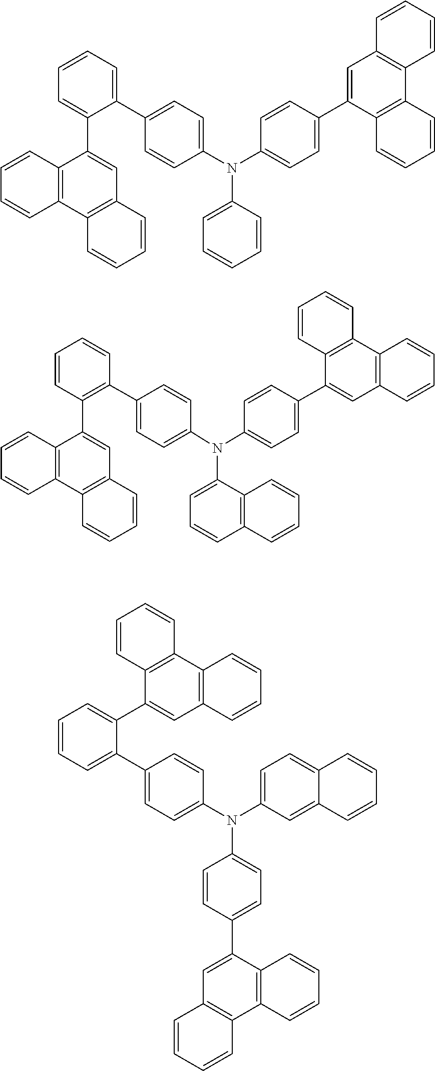 [SMILES: c1ccc(N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cc5ccccc5c5ccccc45)cc3)c3cccc4ccccc34)cc2)c1 ; c1ccc(-c2cc3ccccc3c3ccccc23)c(-c2ccc(N(c3ccc(-c4cc5ccccc5c5ccccc45)cc3)c3ccc4ccccc4c3)cc2)c1] 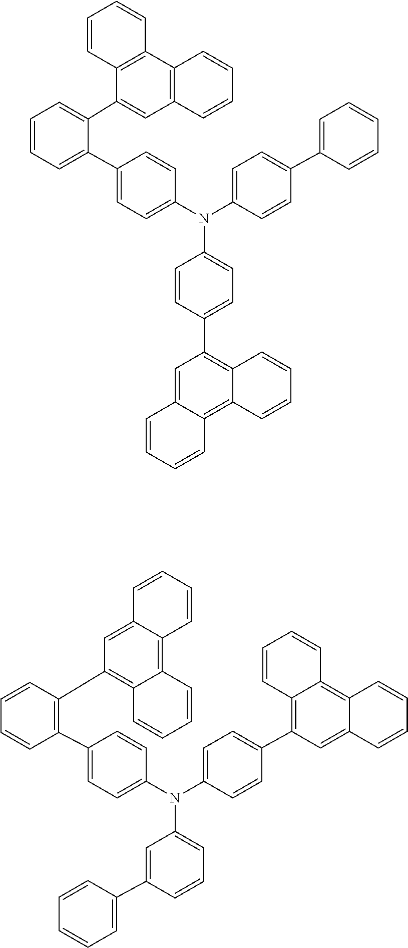 [SMILES: c1ccc(-c2ccc(N(c3ccc(-c4ccccc4-c4cc5ccccc5c5ccccc45)cc3)c3ccc(-c4cc5ccccc5c5ccccc45)cc3)cc2)cc1 ; c1ccc(-c2cccc(N(c3ccc(-c4ccccc4-c4cc5ccccc5c5ccccc45)cc3)c3ccc(-c4cc5ccccc5c5ccccc45)cc3)c2)cc1] 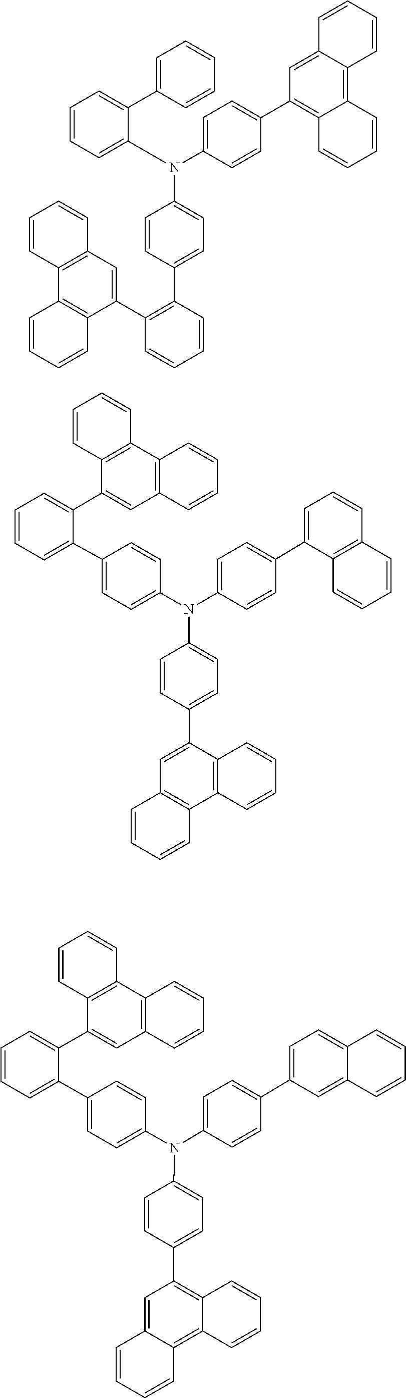 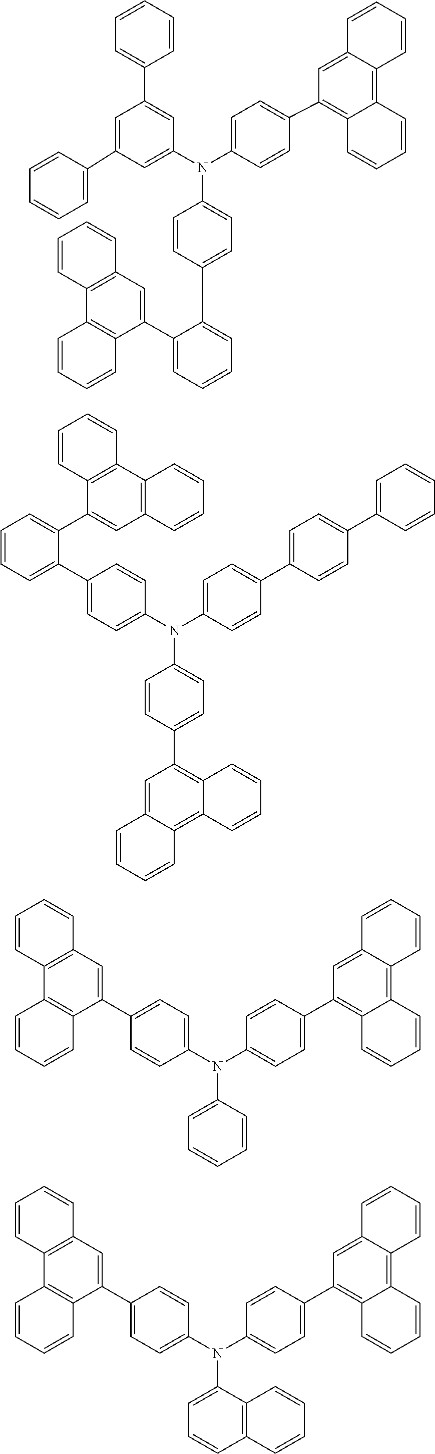 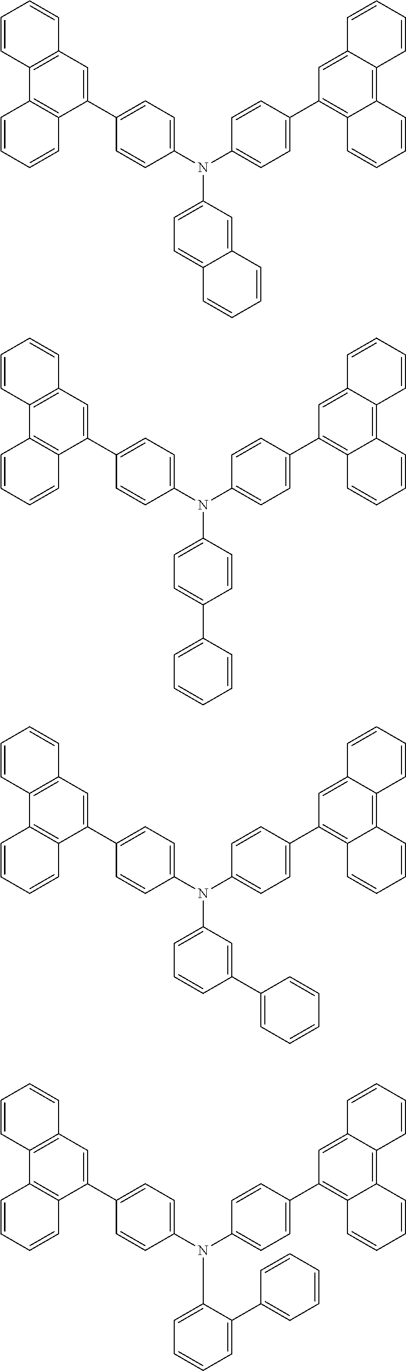 [SMILES: c1ccc2cc(N(c3ccc(-c4cc5ccccc5c5ccccc45)cc3)c3ccc(-c4cc5ccccc5c5ccccc45)cc3)ccc2c1 ; c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2cccc(N(c3ccc(-c4cc5ccccc5c5ccccc45)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cc4ccccc4c4ccccc34)cc2)cc1] 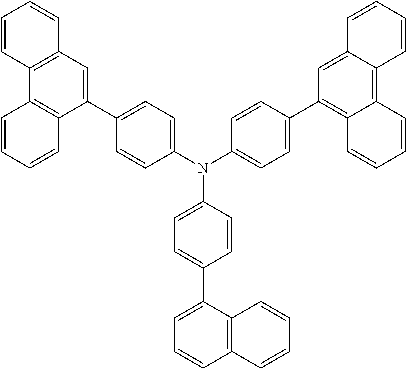 [SMILES: c1ccc2c(-c3ccc(N(c4ccc(-c5cc6ccccc6c6ccccc56)cc4)c4ccc(-c5cc6ccccc6c6ccccc56)cc4)cc3)cccc2c1] 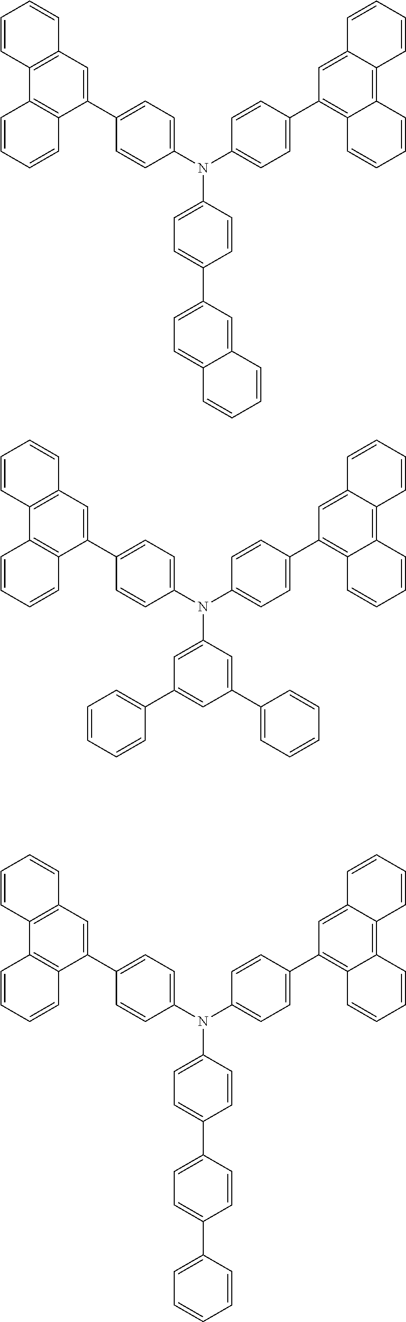 [SMILES: c1ccc2cc(-c3ccc(N(c4ccc(-c5cc6ccccc6c6ccccc56)cc4)c4ccc(-c5cc6ccccc6c6ccccc56)cc4)cc3)ccc2c1 ; c1ccc(-c2cc(-c3ccccc3)cc(N(c3ccc(-c4cc5ccccc5c5ccccc45)cc3)c3ccc(-c4cc5ccccc5c5ccccc45)cc3)c2)cc1 ; c1ccc(-c2ccc(-c3ccc(N(c4ccc(-c5cc6ccccc6c6ccccc56)cc4)c4ccc(-c5cc6ccccc6c6ccccc56)cc4)cc3)cc2)cc1] 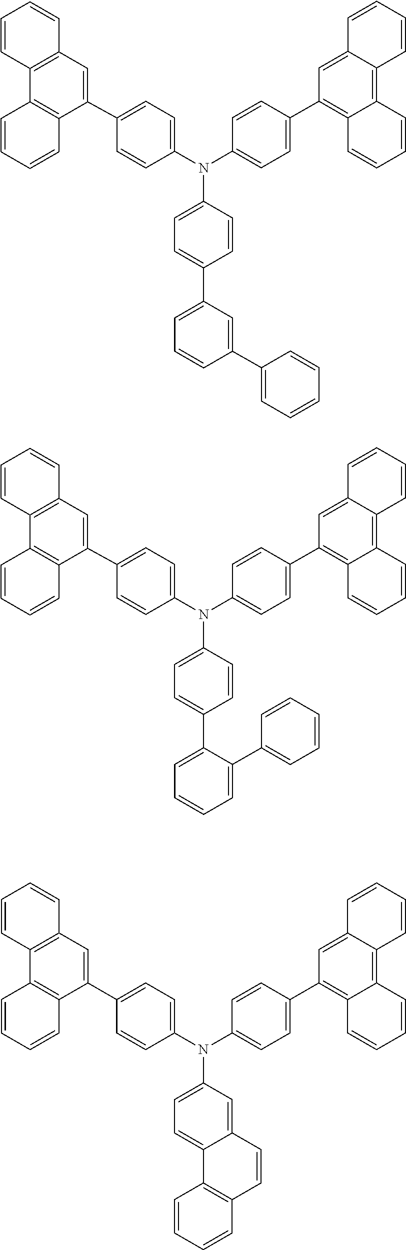 [SMILES: c1ccc(-c2cccc(-c3ccc(N(c4ccc(-c5cc6ccccc6c6ccccc56)cc4)c4ccc(-c5cc6ccccc6c6ccccc56)cc4)cc3)c2)cc1 ; c1ccc(-c2ccccc2-c2ccc(N(c3ccc(-c4cc5ccccc5c5ccccc45)cc3)c3ccc(-c4cc5ccccc5c5ccccc45)cc3)cc2)cc1 ; c1ccc2c(c1)ccc1cc(N(c3ccc(-c4cc5ccccc5c5ccccc45)cc3)c3ccc(-c4cc5ccccc5c5ccccc45)cc3)ccc12] 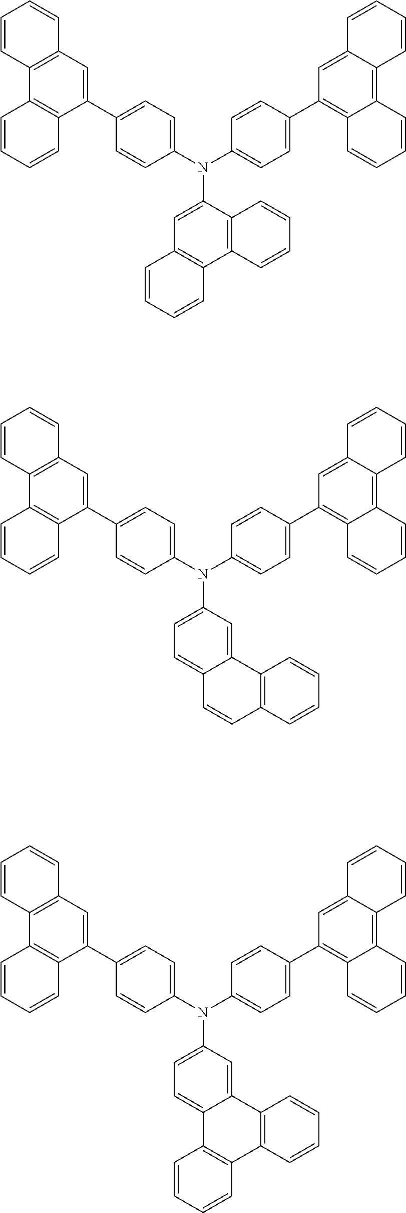 [SMILES: c1ccc2c(c1)cc(-c1ccc(N(c3ccc(-c4cc5ccccc5c5ccccc45)cc3)c3cc4ccccc4c4ccccc34)cc1)c1ccccc12 ; c1ccc2c(c1)ccc1ccc(N(c3ccc(-c4cc5ccccc5c5ccccc45)cc3)c3ccc(-c4cc5ccccc5c5ccccc45)cc3)cc12 ; c1ccc2c(c1)cc(-c1ccc(N(c3ccc(-c4cc5ccccc5c5ccccc45)cc3)c3ccc4c5ccccc5c5ccccc5c4c3)cc1)c1ccccc12] 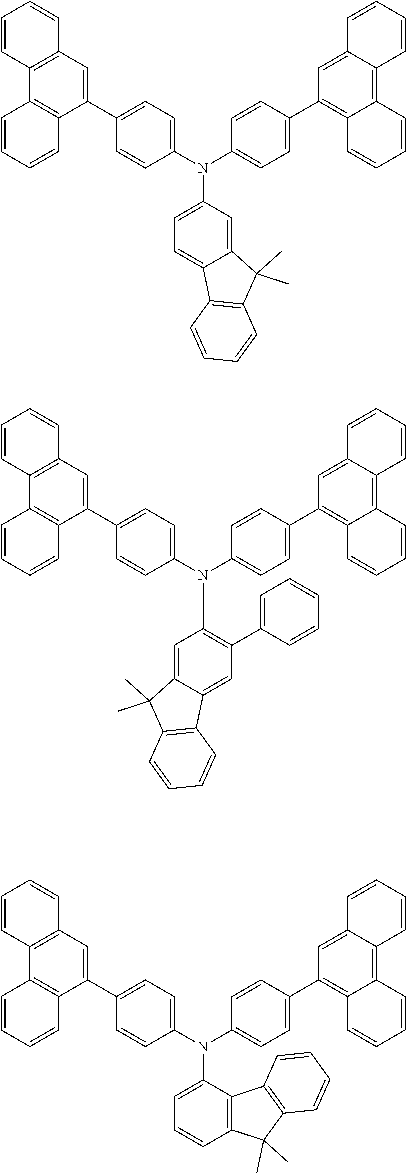 [SMILES: CC1(C)c2ccccc2-c2ccc(N(c3ccc(-c4cc5ccccc5c5ccccc45)cc3)c3ccc(-c4cc5ccccc5c5ccccc45)cc3)cc21 ; CC1(C)c2ccccc2-c2cc(-c3ccccc3)c(N(c3ccc(-c4cc5ccccc5c5ccccc45)cc3)c3ccc(-c4cc5ccccc5c5ccccc45)cc3)cc21 ; CC1(C)c2ccccc2-c2c(N(c3ccc(-c4cc5ccccc5c5ccccc45)cc3)c3ccc(-c4cc5ccccc5c5ccccc45)cc3)cccc21] 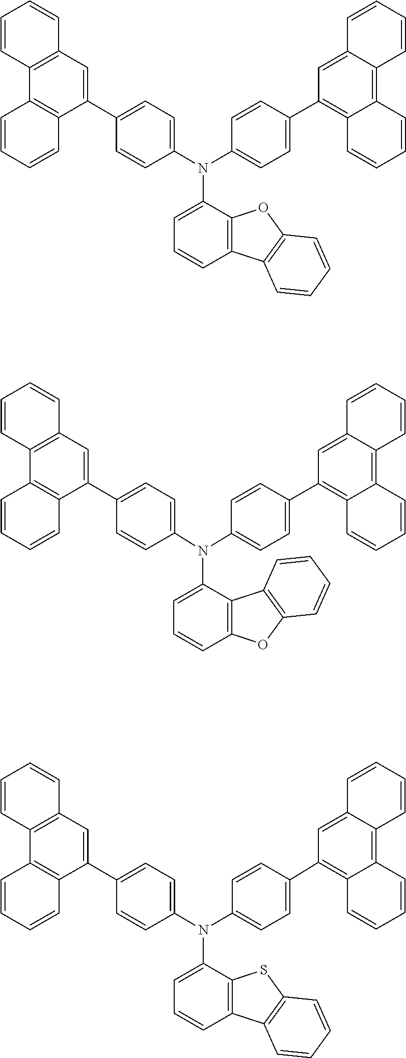 [SMILES: c1ccc2c(c1)cc(-c1ccc(N(c3ccc(-c4cc5ccccc5c5ccccc45)cc3)c3cccc4c3oc3ccccc34)cc1)c1ccccc12 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc2c(c1)cc(-c1ccc(N(c3ccc(-c4cc5ccccc5c5ccccc45)cc3)c3cccc4c3sc3ccccc34)cc1)c1ccccc12] 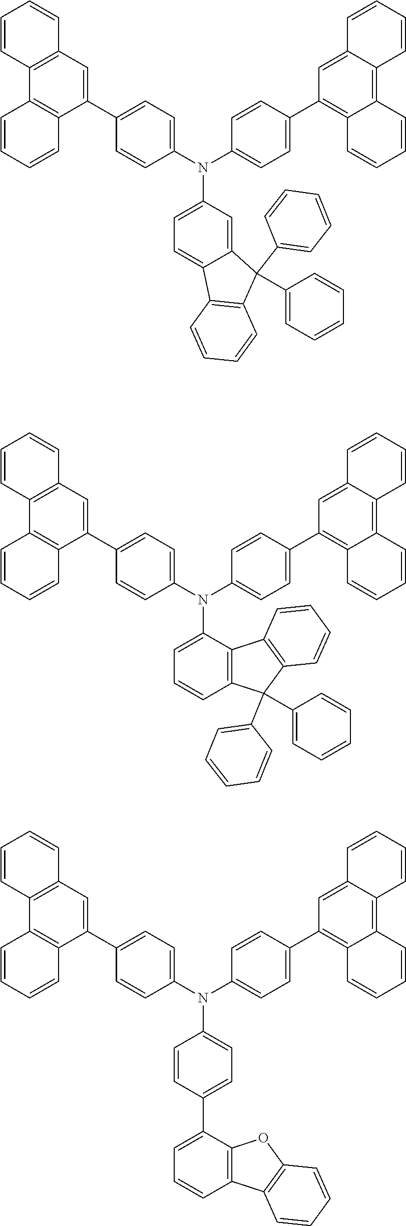 [SMILES: c1ccc(C2(c3ccccc3)c3ccccc3-c3ccc(N(c4ccc(-c5cc6ccccc6c6ccccc56)cc4)c4ccc(-c5cc6ccccc6c6ccccc56)cc4)cc32)cc1 ; c1ccc(C2(c3ccccc3)c3ccccc3-c3c(N(c4ccc(-c5cc6ccccc6c6ccccc56)cc4)c4ccc(-c5cc6ccccc6c6ccccc56)cc4)cccc32)cc1 ; c1ccc2c(c1)cc(-c1ccc(N(c3ccc(-c4cc5ccccc5c5ccccc45)cc3)c3ccc(-c4cccc5c4oc4ccccc45)cc3)cc1)c1ccccc12] 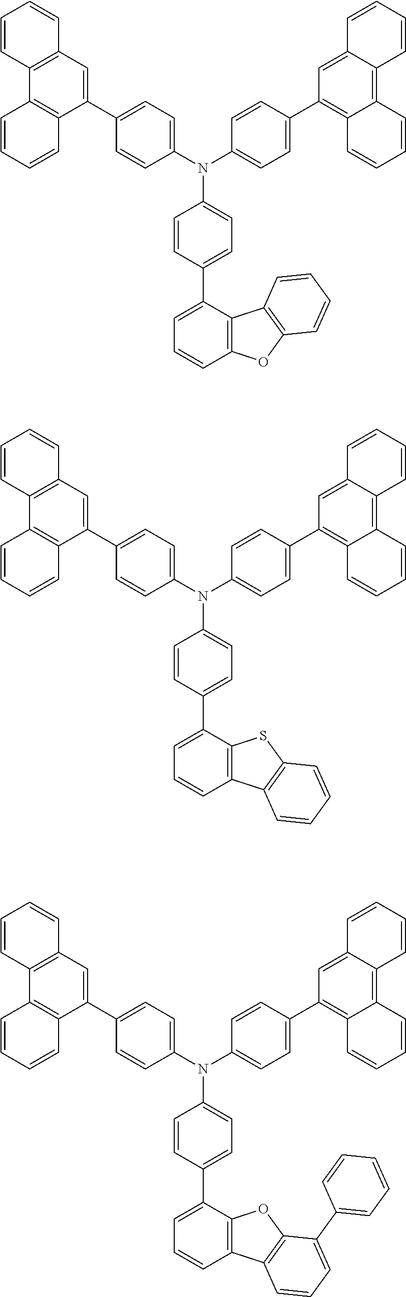 [SMILES: c1ccc2c(c1)cc(-c1ccc(N(c3ccc(-c4cc5ccccc5c5ccccc45)cc3)c3ccc(-c4cccc5oc6ccccc6c45)cc3)cc1)c1ccccc12 ; c1ccc2c(c1)cc(-c1ccc(N(c3ccc(-c4cc5ccccc5c5ccccc45)cc3)c3ccc(-c4cccc5c4sc4ccccc45)cc3)cc1)c1ccccc12 ; c1ccc(-c2cccc3c2oc2c(-c4ccc(N(c5ccc(-c6cc7ccccc7c7ccccc67)cc5)c5ccc(-c6cc7ccccc7c7ccccc67)cc5)cc4)cccc23)cc1] 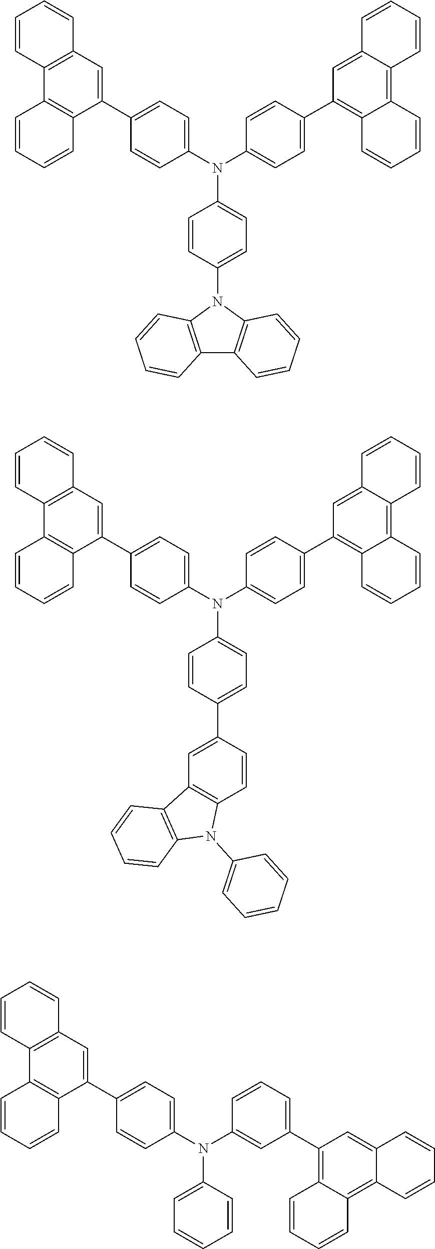 [SMILES: c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12 ; c1ccc(-n2c3ccccc3c3cc(-c4ccc(N(c5ccc(-c6cc7ccccc7c7ccccc67)cc5)c5ccc(-c6cc7ccccc7c7ccccc67)cc5)cc4)ccc32)cc1 ; c1ccc(N(c2ccc(-c3cc4ccccc4c4ccccc34)cc2)c2cccc(-c3cc4ccccc4c4ccccc34)c2)cc1] 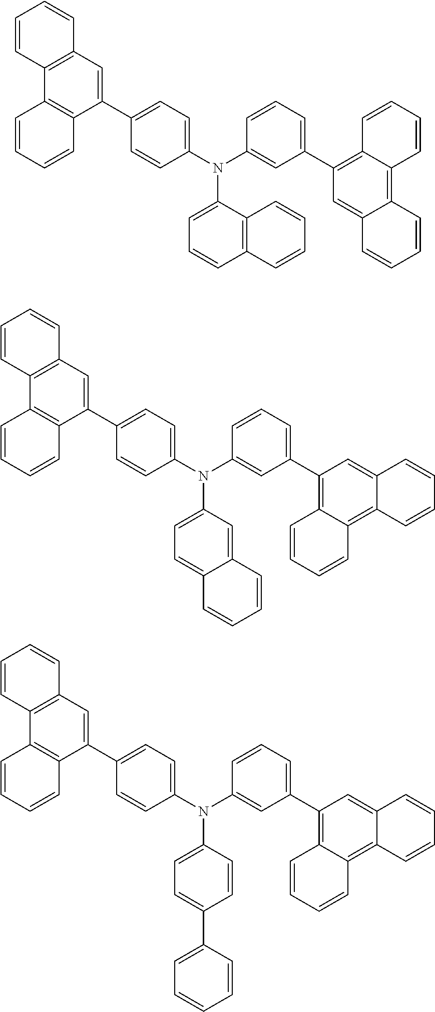 [SMILES: c1cc(-c2cc3ccccc3c3ccccc23)cc(N(c2ccc(-c3cc4ccccc4c4ccccc34)cc2)c2cccc3ccccc23)c1 ; c1cc(-c2cc3ccccc3c3ccccc23)cc(N(c2ccc(-c3cc4ccccc4c4ccccc34)cc2)c2ccc3ccccc3c2)c1 ; c1ccc(-c2ccc(N(c3ccc(-c4cc5ccccc5c5ccccc45)cc3)c3cccc(-c4cc5ccccc5c5ccccc45)c3)cc2)cc1] 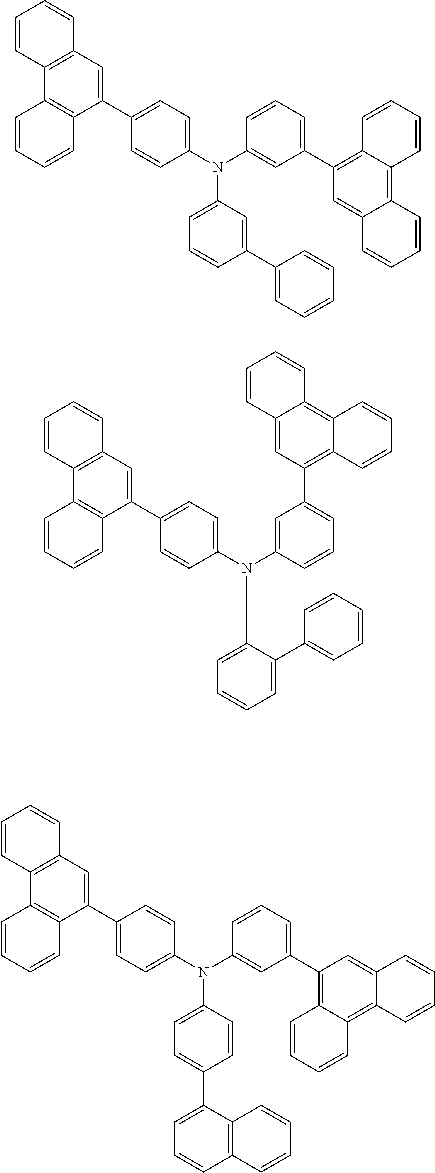 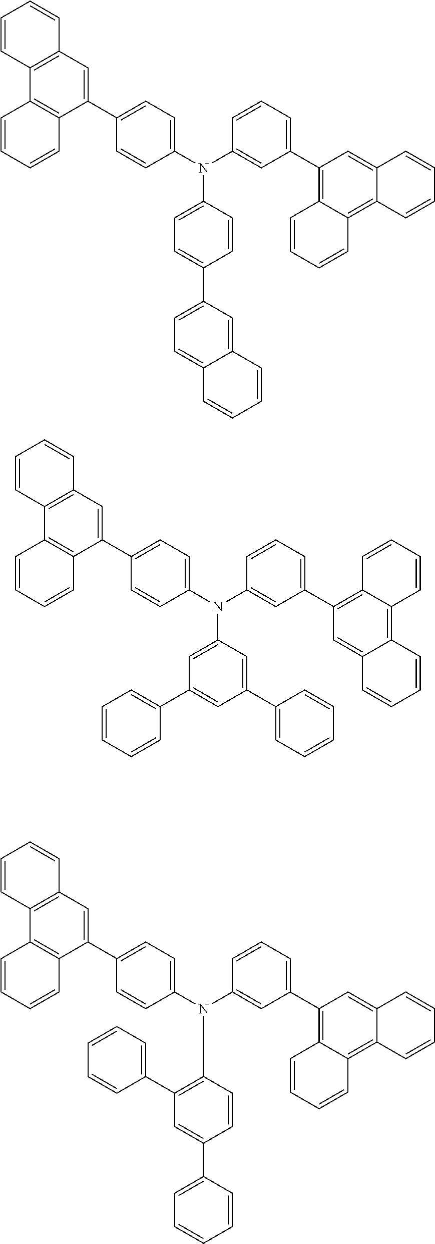 [SMILES: c1cc(-c2cc3ccccc3c3ccccc23)cc(N(c2ccc(-c3ccc4ccccc4c3)cc2)c2ccc(-c3cc4ccccc4c4ccccc34)cc2)c1 ; c1ccc(-c2cc(-c3ccccc3)cc(N(c3ccc(-c4cc5ccccc5c5ccccc45)cc3)c3cccc(-c4cc5ccccc5c5ccccc45)c3)c2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1] 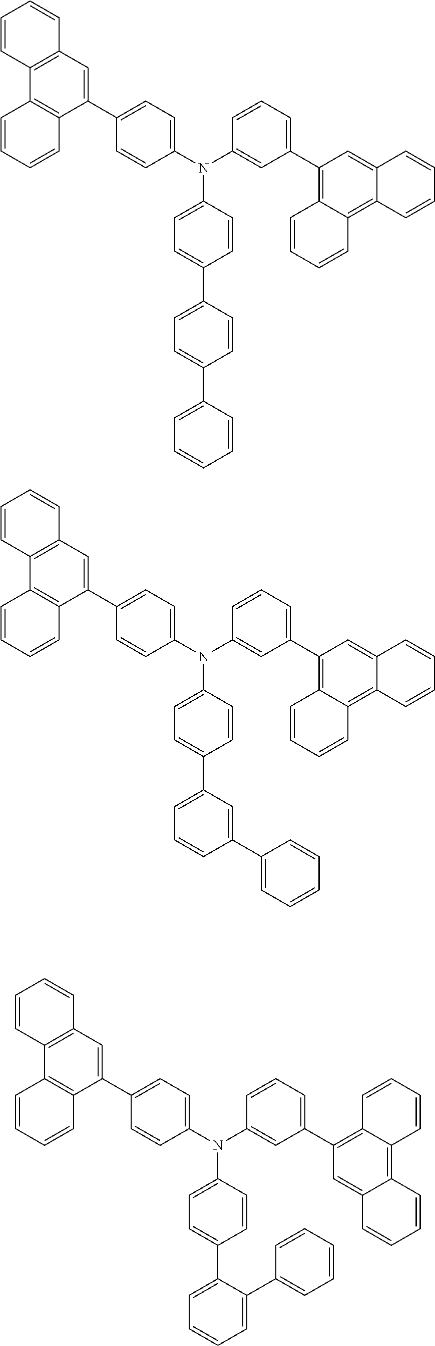 [SMILES: c1ccc(-c2ccc(-c3ccc(N(c4ccc(-c5cc6ccccc6c6ccccc56)cc4)c4cccc(-c5cc6ccccc6c6ccccc56)c4)cc3)cc2)cc1 ; c1ccc(-c2cccc(-c3ccc(N(c4ccc(-c5cc6ccccc6c6ccccc56)cc4)c4cccc(-c5cc6ccccc6c6ccccc56)c4)cc3)c2)cc1 ; c1ccc(-c2ccccc2-c2ccc(N(c3ccc(-c4cc5ccccc5c5ccccc45)cc3)c3cccc(-c4cc5ccccc5c5ccccc45)c3)cc2)cc1] 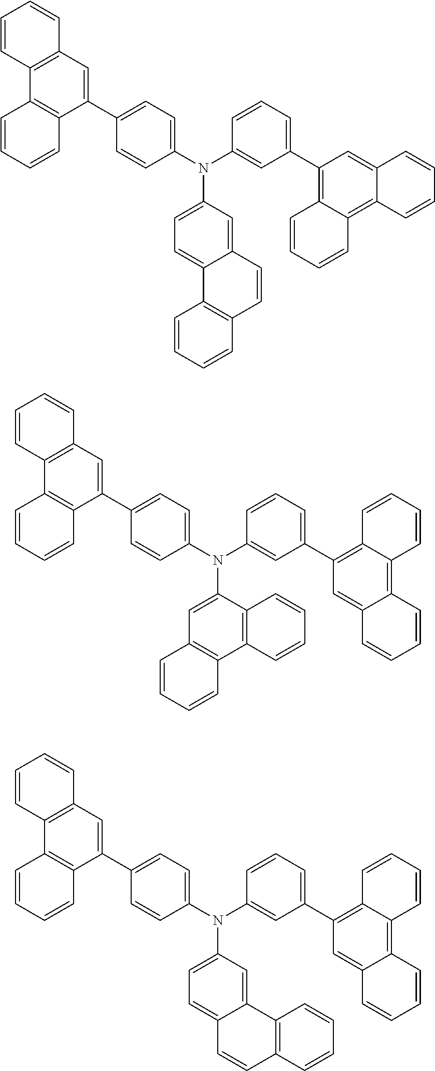 [SMILES: c1cc(-c2cc3ccccc3c3ccccc23)cc(N(c2ccc(-c3cc4ccccc4c4ccccc34)cc2)c2ccc3c(ccc4ccccc43)c2)c1 ; c1cc(-c2cc3ccccc3c3ccccc23)cc(N(c2ccc(-c3cc4ccccc4c4ccccc34)cc2)c2cc3ccccc3c3ccccc23)c1 ; c1cc(-c2cc3ccccc3c3ccccc23)cc(N(c2ccc(-c3cc4ccccc4c4ccccc34)cc2)c2ccc3ccc4ccccc4c3c2)c1] 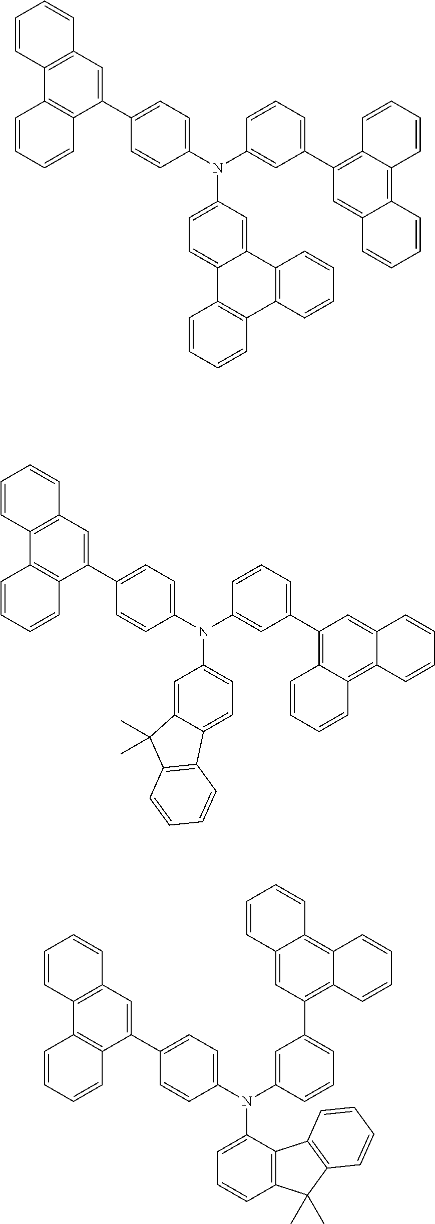 [SMILES: c1cc(-c2cc3ccccc3c3ccccc23)cc(N(c2ccc(-c3cc4ccccc4c4ccccc34)cc2)c2ccc3c4ccccc4c4ccccc4c3c2)c1 ; CC1(C)c2ccccc2-c2ccc(N(c3ccc(-c4cc5ccccc5c5ccccc45)cc3)c3cccc(-c4cc5ccccc5c5ccccc45)c3)cc21 ; CC1(C)c2ccccc2-c2c(N(c3ccc(-c4cc5ccccc5c5ccccc45)cc3)c3cccc(-c4cc5ccccc5c5ccccc45)c3)cccc21] 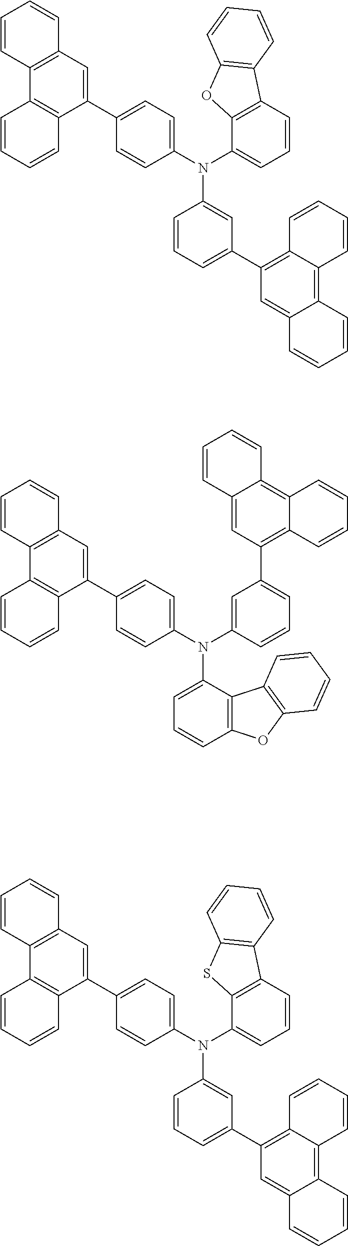 [SMILES: c1cc(-c2cc3ccccc3c3ccccc23)cc(N(c2ccc(-c3cc4ccccc4c4ccccc34)cc2)c2cccc3c2oc2ccccc23)c1 ; c1cc(-c2cc3ccccc3c3ccccc23)cc(N(c2ccc(-c3cc4ccccc4c4ccccc34)cc2)c2cccc3oc4ccccc4c23)c1 ; c1cc(-c2cc3ccccc3c3ccccc23)cc(N(c2ccc(-c3cc4ccccc4c4ccccc34)cc2)c2cccc3c2sc2ccccc23)c1] 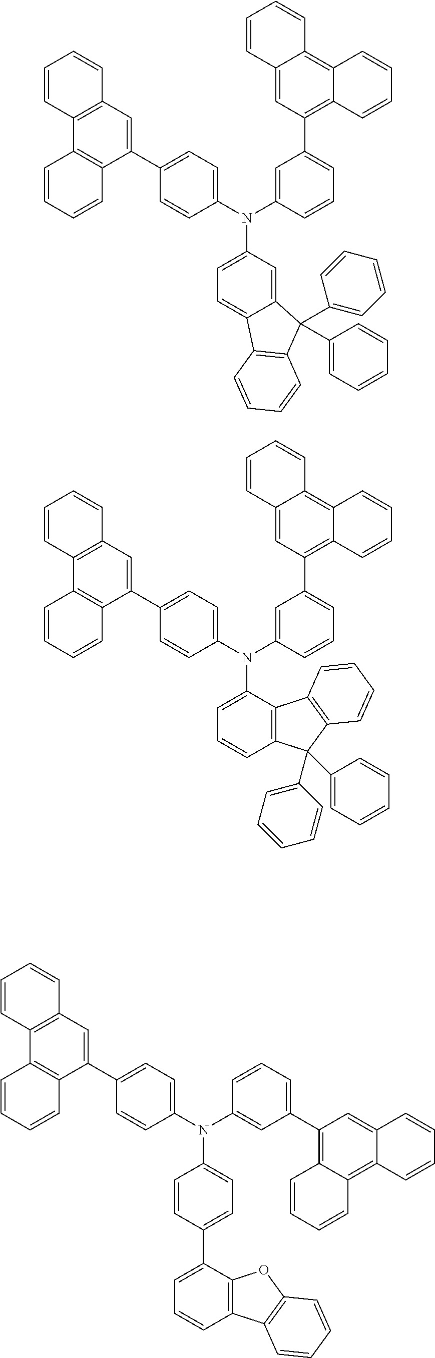 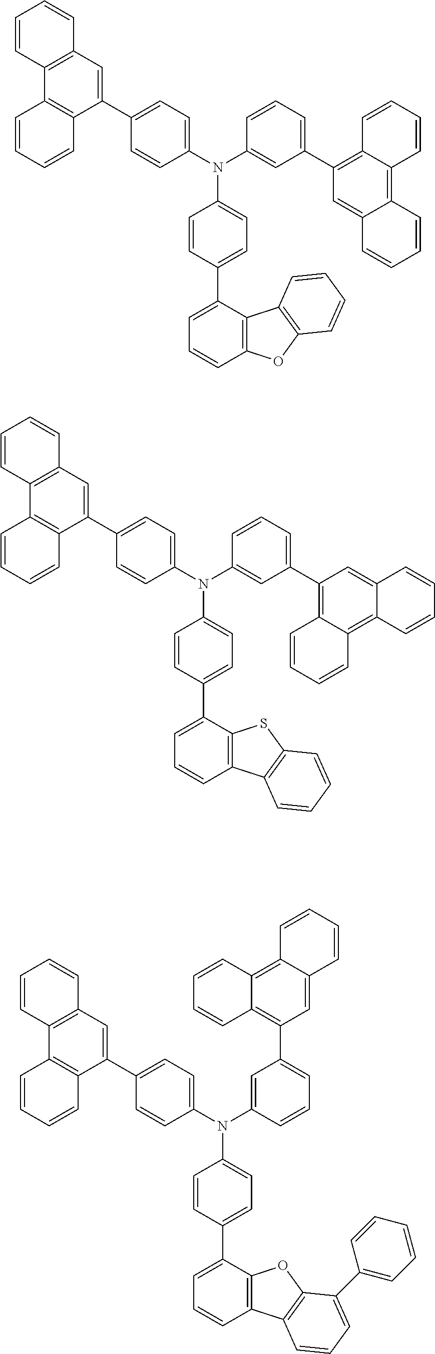 [SMILES: c1cc(-c2cc3ccccc3c3ccccc23)cc(N(c2ccc(-c3cc4ccccc4c4ccccc34)cc2)c2ccc(-c3cccc4oc5ccccc5c34)cc2)c1 ; c1cc(-c2cc3ccccc3c3ccccc23)cc(N(c2ccc(-c3cc4ccccc4c4ccccc34)cc2)c2ccc(-c3cccc4c3sc3ccccc34)cc2)c1 ; c1ccc(-c2cccc3c2oc2c(-c4ccc(N(c5ccc(-c6cc7ccccc7c7ccccc67)cc5)c5cccc(-c6cc7ccccc7c7ccccc67)c5)cc4)cccc23)cc1] 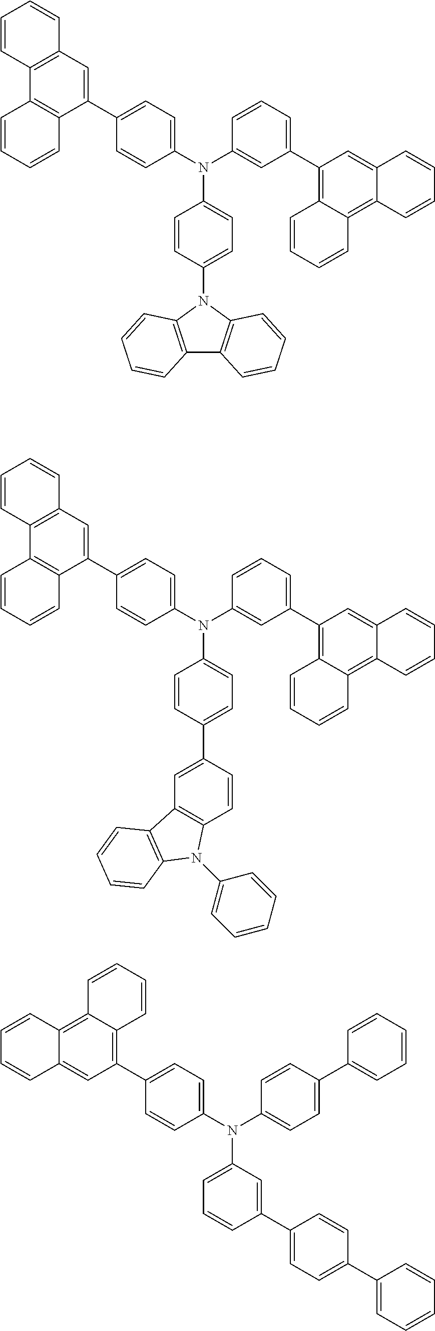 [SMILES: c1cc(-c2cc3ccccc3c3ccccc23)cc(N(c2ccc(-c3cc4ccccc4c4ccccc34)cc2)c2ccc(-n3c4ccccc4c4ccccc43)cc2)c1 ; c1ccc(-n2c3ccccc3c3cc(-c4ccc(N(c5ccc(-c6cc7ccccc7c7ccccc67)cc5)c5cccc(-c6cc7ccccc7c7ccccc67)c5)cc4)ccc32)cc1 ; c1ccc(-c2ccc(-c3cccc(N(c4ccc(-c5ccccc5)cc4)c4ccc(-c5cc6ccccc6c6ccccc56)cc4)c3)cc2)cc1] 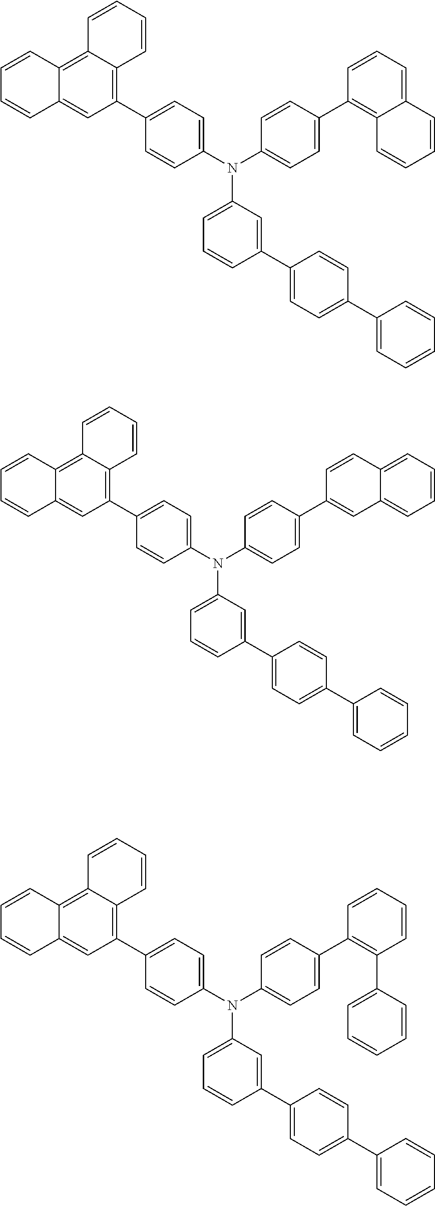 [SMILES: c1ccc(-c2ccc(-c3cccc(N(c4ccc(-c5cccc6ccccc56)cc4)c4ccc(-c5cc6ccccc6c6ccccc56)cc4)c3)cc2)cc1 ; c1ccc(-c2ccc(-c3cccc(N(c4ccc(-c5ccc6ccccc6c5)cc4)c4ccc(-c5cc6ccccc6c6ccccc56)cc4)c3)cc2)cc1 ; c1ccc(-c2ccc(-c3cccc(N(c4ccc(-c5ccccc5-c5ccccc5)cc4)c4ccc(-c5cc6ccccc6c6ccccc56)cc4)c3)cc2)cc1] 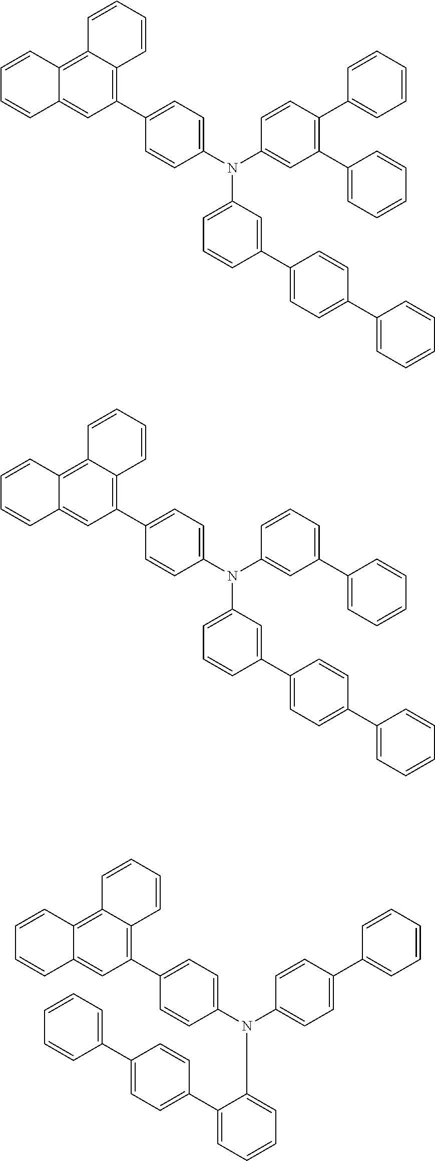 [SMILES: c1ccc(-c2ccc(-c3cccc(N(c4ccc(-c5cc6ccccc6c6ccccc56)cc4)c4ccc(-c5ccccc5)c(-c5ccccc5)c4)c3)cc2)cc1 ; c1ccc(-c2ccc(-c3cccc(N(c4ccc(-c5cc6ccccc6c6ccccc56)cc4)c4cccc(-c5ccccc5)c4)c3)cc2)cc1 ; c1ccc(-c2ccc(-c3ccccc3N(c3ccc(-c4ccccc4)cc3)c3ccc(-c4cc5ccccc5c5ccccc45)cc3)cc2)cc1] 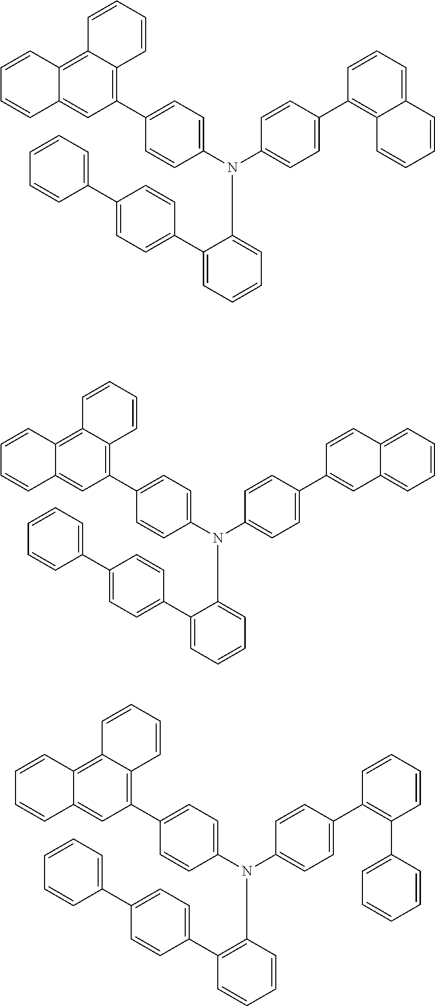 [SMILES: c1ccc(-c2ccc(-c3ccccc3N(c3ccc(-c4cccc5ccccc45)cc3)c3ccc(-c4cc5ccccc5c5ccccc45)cc3)cc2)cc1 ; c1ccc(-c2ccc(-c3ccccc3N(c3ccc(-c4ccc5ccccc5c4)cc3)c3ccc(-c4cc5ccccc5c5ccccc45)cc3)cc2)cc1 ; c1ccc(-c2ccc(-c3ccccc3N(c3ccc(-c4ccccc4-c4ccccc4)cc3)c3ccc(-c4cc5ccccc5c5ccccc45)cc3)cc2)cc1] 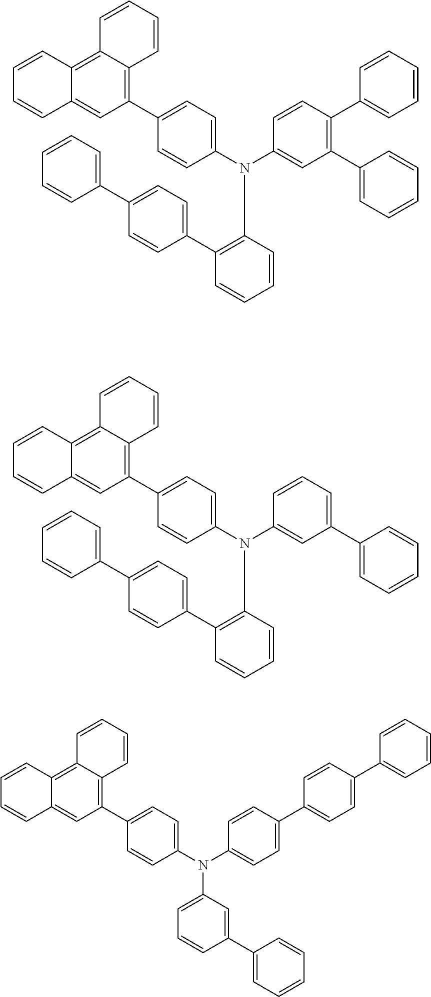 [SMILES: c1ccc(-c2ccc(-c3ccccc3N(c3ccc(-c4cc5ccccc5c5ccccc45)cc3)c3ccc(-c4ccccc4)c(-c4ccccc4)c3)cc2)cc1 ; c1ccc(-c2ccc(-c3ccccc3N(c3ccc(-c4cc5ccccc5c5ccccc45)cc3)c3cccc(-c4ccccc4)c3)cc2)cc1 ; c1ccc(-c2ccc(-c3ccc(N(c4ccc(-c5cc6ccccc6c6ccccc56)cc4)c4cccc(-c5ccccc5)c4)cc3)cc2)cc1] 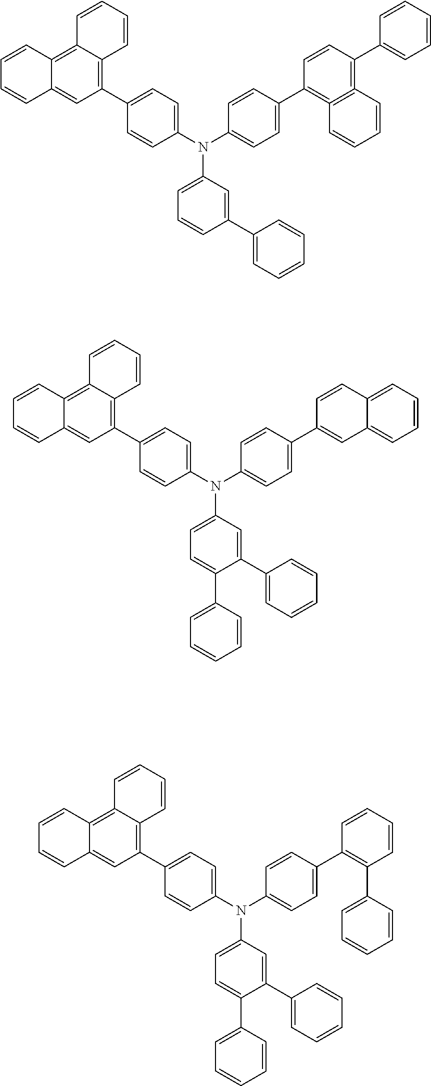 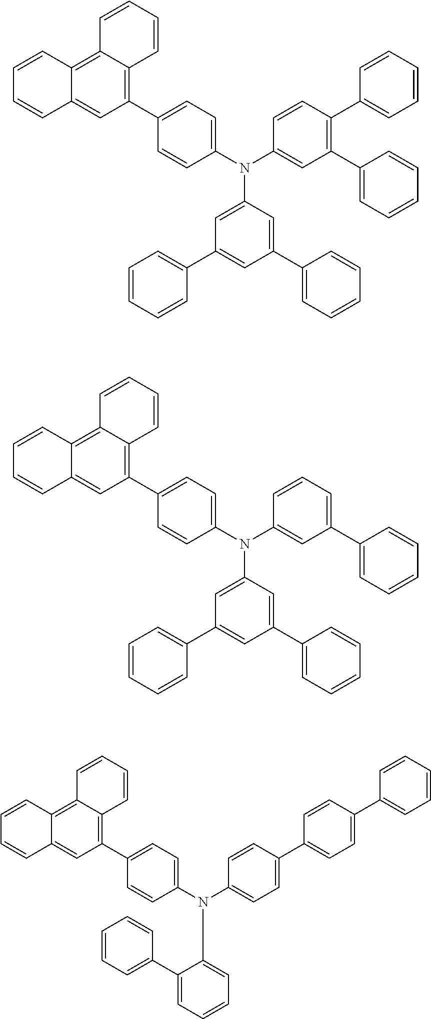 [SMILES: c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2ccc(-c3ccc(N(Cc4ccccc4-c4ccccc4)c4ccc(-c5cc6ccccc6c6ccccc56)cc4)cc3)cc2)cc1] 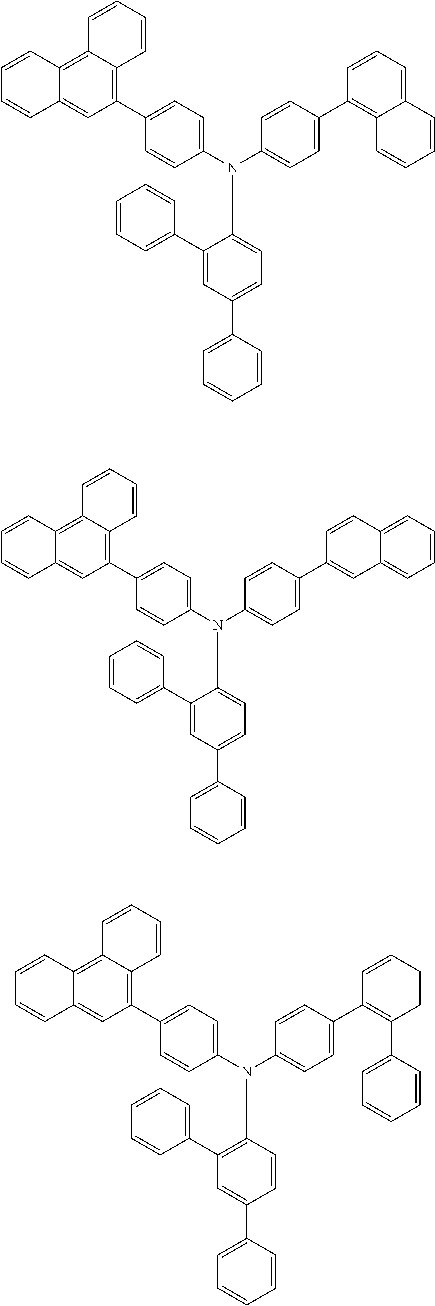 [SMILES: c1ccc(-c2ccc(CN(c3ccc(-c4cccc5ccccc45)cc3)c3ccc(-c4cc5ccccc5c5ccccc45)cc3)c(-c3ccccc3)c2)cc1 ; c1ccc(-c2ccc(CN(c3ccc(-c4ccc5ccccc5c4)cc3)c3ccc(-c4cc5ccccc5c5ccccc45)cc3)c(-c3ccccc3)c2)cc1 ; C1=CC(c2ccc(N(Cc3ccc(-c4ccccc4)cc3-c3ccccc3)c3ccc(-c4cc5ccccc5c5ccccc45)cc3)cc2)=C(c2ccccc2)CC1] 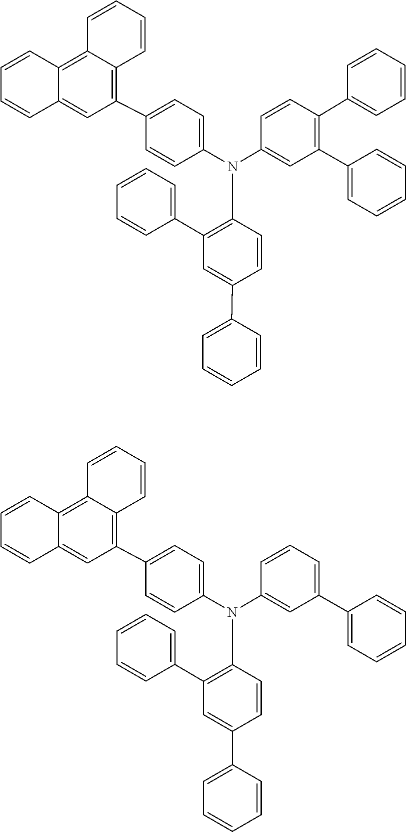 [SMILES: c1ccc(-c2ccc(N(c3ccc(-c4cc5ccccc5c5ccccc45)cc3)c3ccc(-c4ccccc4)c(-c4ccccc4)c3)c(-c3ccccc3)c2)cc1 ; c1ccc(-c2cccc(N(c3ccc(-c4cc5ccccc5c5ccccc45)cc3)c3ccc(-c4ccccc4)cc3-c3ccccc3)c2)cc1] 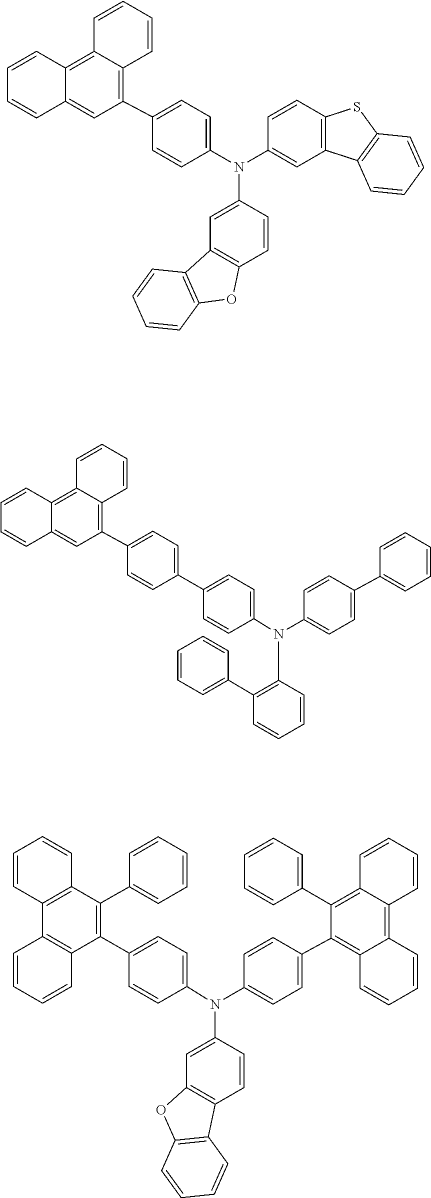 [SMILES: c1ccc2c(c1)cc(-c1ccc(N(c3ccc4oc5ccccc5c4c3)c3ccc4sc5ccccc5c4c3)cc1)c1ccccc12 ; c1ccc(-c2ccc(N(c3ccc(-c4ccc(-c5cc6ccccc6c6ccccc56)cc4)cc3)c3ccccc3-c3ccccc3)cc2)cc1 ; c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1] 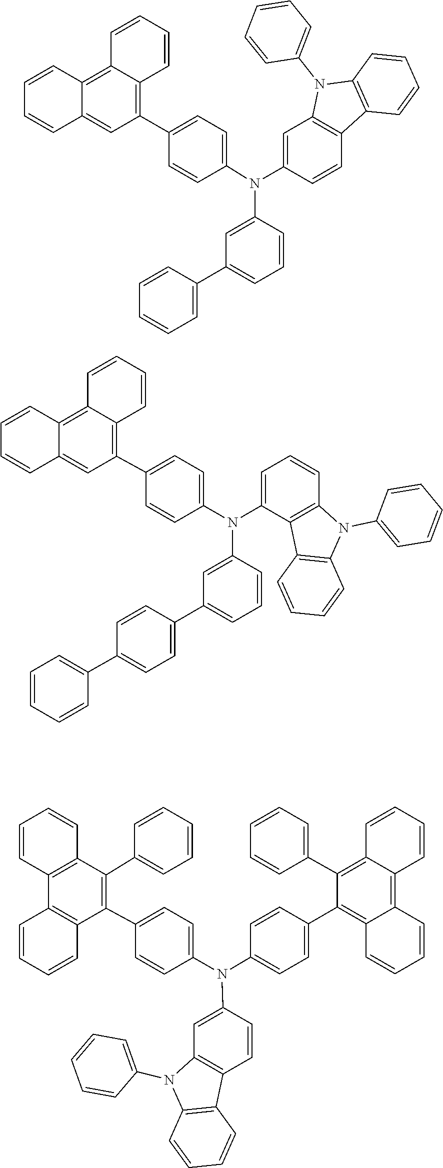 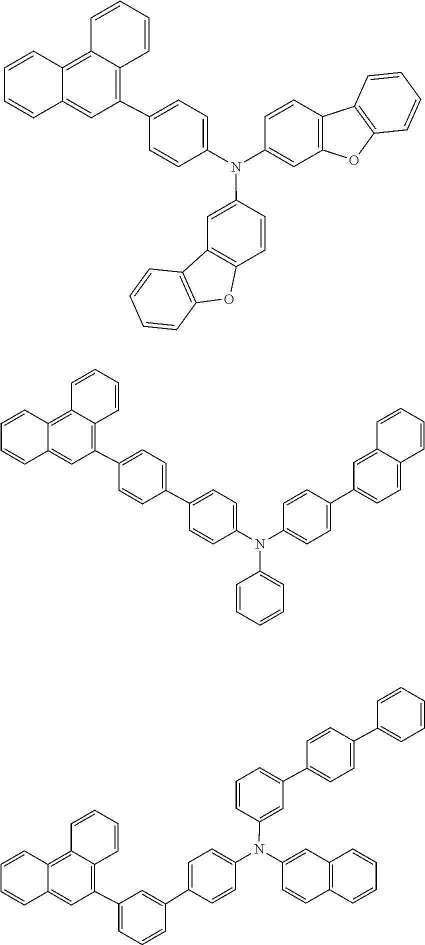 [SMILES: c1ccc2c(c1)cc(-c1ccc(N(c3ccc4c(c3)oc3ccccc34)c3ccc4oc5ccccc5c4c3)cc1)c1ccccc12 ; c1ccc(N(c2ccc(-c3ccc(-c4cc5ccccc5c5ccccc45)cc3)cc2)c2ccc(-c3ccc4ccccc4c3)cc2)cc1 ; c1ccc(-c2ccc(-c3cccc(N(c4ccc(-c5cccc(-c6cc7ccccc7c7ccccc67)c5)cc4)c4ccc5ccccc5c4)c3)cc2)cc1] 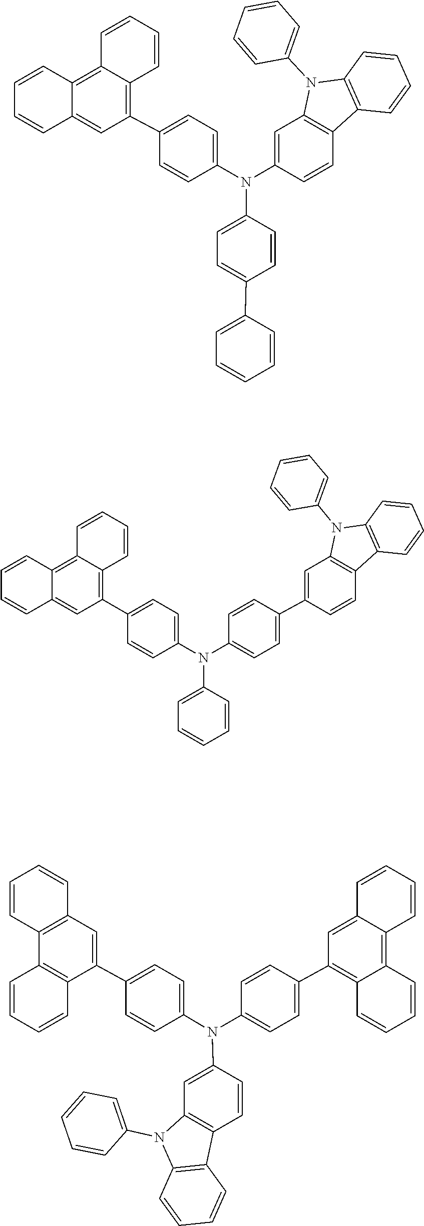 [SMILES: c1ccc(-c2ccc(N(c3ccc(-c4cc5ccccc5c5ccccc45)cc3)c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cc4ccccc4c4ccccc34)cc2)cc1 ; c1ccc(-n2c3ccccc3c3ccc(N(c4ccc(-c5cc6ccccc6c6ccccc56)cc4)c4ccc(-c5cc6ccccc6c6ccccc56)cc4)cc32)cc1] 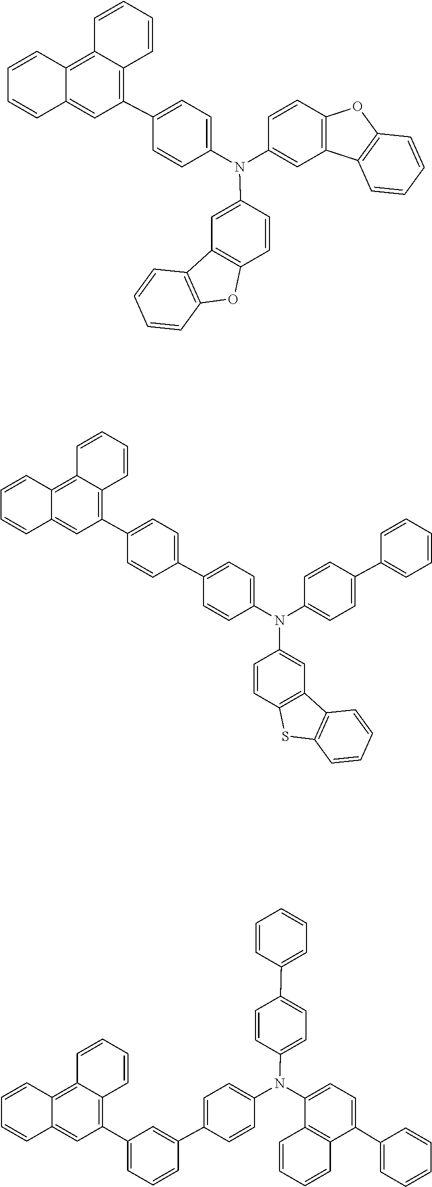 [SMILES: c1ccc2c(c1)cc(-c1ccc(N(c3ccc4oc5ccccc5c4c3)c3ccc4oc5ccccc5c4c3)cc1)c1ccccc12 ; c1ccc(-c2ccc(N(c3ccc(-c4ccc(-c5cc6ccccc6c6ccccc56)cc4)cc3)c3ccc4sc5ccccc5c4c3)cc2)cc1 ; c1ccc(-c2ccc(N(c3ccc(-c4cccc(-c5cc6ccccc6c6ccccc56)c4)cc3)c3ccc(-c4ccccc4)c4ccccc34)cc2)cc1] 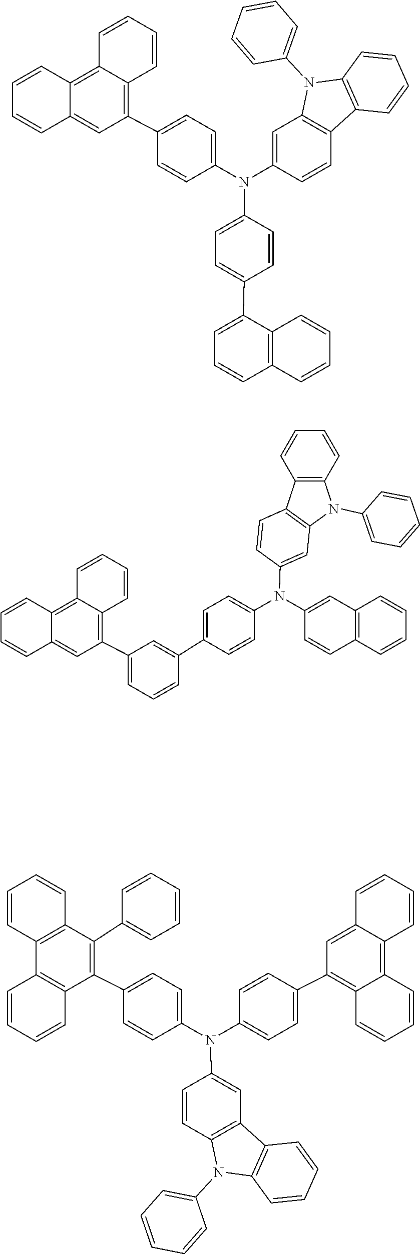 [SMILES: c1ccc(-n2c3ccccc3c3ccc(N(c4ccc(-c5cccc6ccccc56)cc4)c4ccc(-c5cc6ccccc6c6ccccc56)cc4)cc32)cc1 ; c1ccc(-n2c3ccccc3c3ccc(N(c4ccc(-c5cccc(-c6cc7ccccc7c7ccccc67)c5)cc4)c4ccc5ccccc5c4)cc32)cc1 ; c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1] 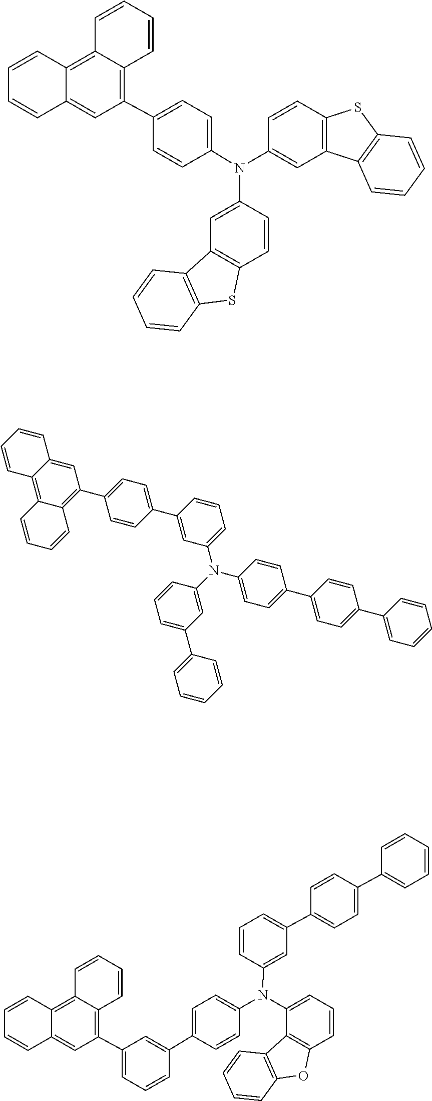 [SMILES: c1ccc2c(c1)cc(-c1ccc(N(c3ccc4sc5ccccc5c4c3)c3ccc4sc5ccccc5c4c3)cc1)c1ccccc12 ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1] 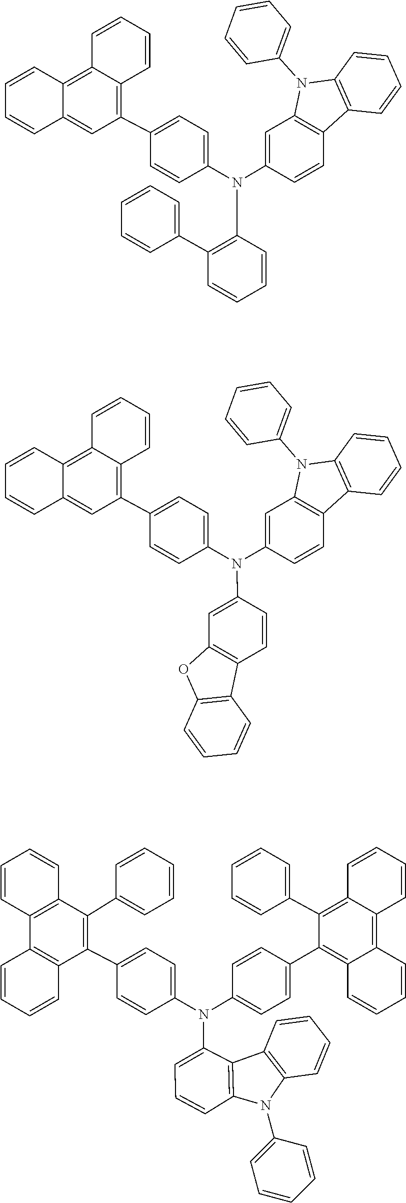 [SMILES: c1ccc(-c2ccccc2N(c2ccc(-c3cc4ccccc4c4ccccc34)cc2)c2ccc3c4ccccc4n(-c4ccccc4)c3c2)cc1 ; c1ccc(-n2c3ccccc3c3ccc(N(c4ccc(-c5cc6ccccc6c6ccccc56)cc4)c4ccc5c(c4)oc4ccccc45)cc32)cc1 ; c1ccc(-c2c(-c3ccc(N(c4ccc(-c5c(-c6ccccc6)c6ccccc6c6ccccc56)cc4)c4cccc5c4c4ccccc4n5-c4ccccc4)cc3)c3ccccc3c3ccccc23)cc1] 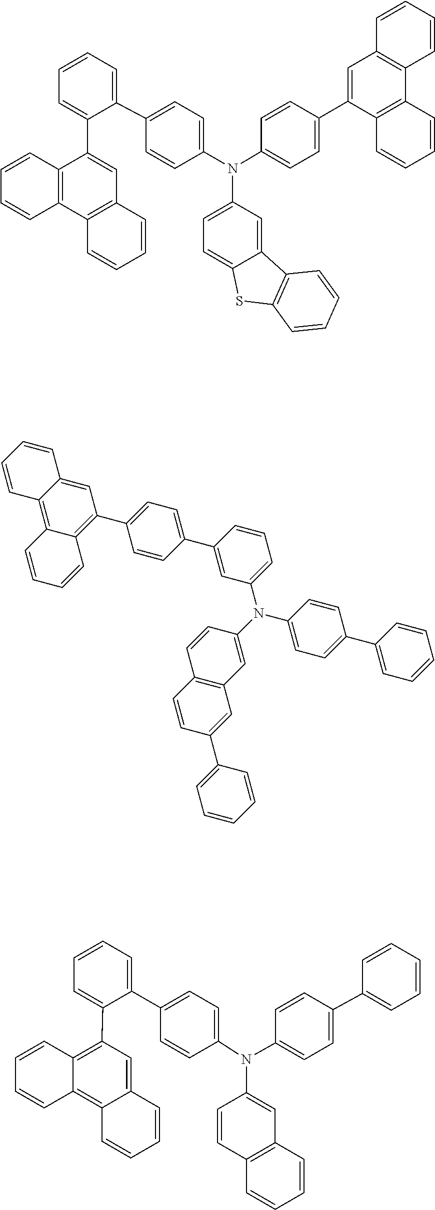 [SMILES: c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1 ; c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1ccc(-c2ccc(N(c3ccc(-c4ccccc4-c4cc5ccccc5c5ccccc45)cc3)c3ccc4ccccc4c3)cc2)cc1] 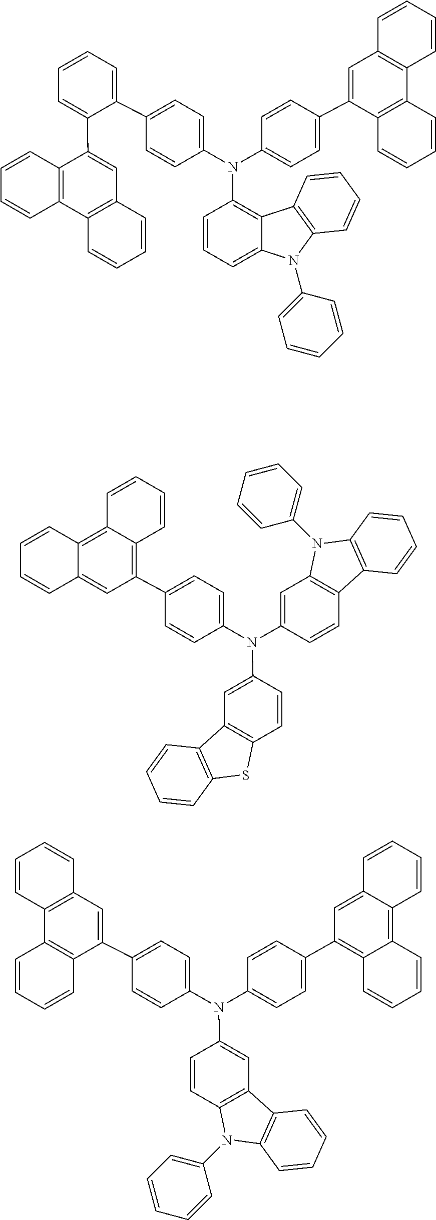 [SMILES: c1ccc(-n2c3ccccc3c3c(N(c4ccc(-c5ccccc5-c5cc6ccccc6c6ccccc56)cc4)c4ccc(-c5cc6ccccc6c6ccccc56)cc4)cccc32)cc1 ; c1ccc(-n2c3ccccc3c3ccc(N(c4ccc(-c5cc6ccccc6c6ccccc56)cc4)c4ccc5sc6ccccc6c5c4)cc32)cc1 ; c1ccc(-n2c3ccccc3c3cc(N(c4ccc(-c5cc6ccccc6c6ccccc56)cc4)c4ccc(-c5cc6ccccc6c6ccccc56)cc4)ccc32)cc1] 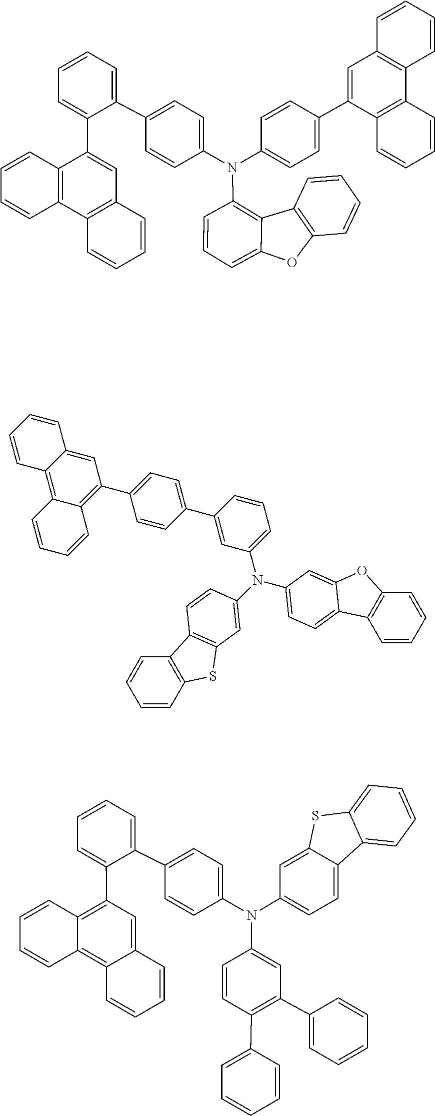 [SMILES: c1ccc(-c2cc3ccccc3c3ccccc23)c(-c2ccc(N(c3ccc(-c4cc5ccccc5c5ccccc45)cc3)c3cccc4oc5ccccc5c34)cc2)c1 ; c1cc(-c2ccc(-c3cc4ccccc4c4ccccc34)cc2)cc(N(c2ccc3c(c2)oc2ccccc23)c2ccc3c(c2)sc2ccccc23)c1 ; c1ccc(-c2ccc(N(c3ccc(-c4ccccc4-c4cc5ccccc5c5ccccc45)cc3)c3ccc4c(c3)sc3ccccc34)cc2-c2ccccc2)cc1] 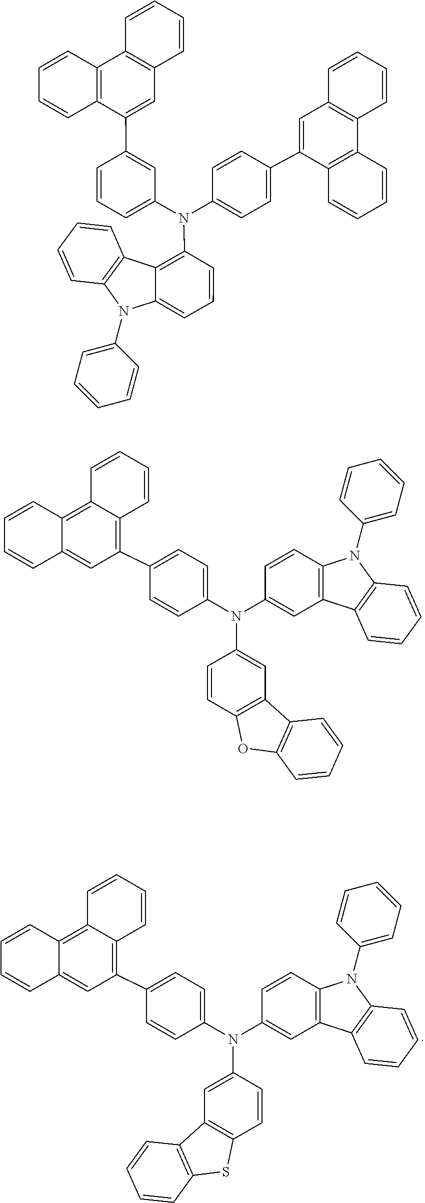 [SMILES: c1ccc(-n2c3ccccc3c3c(N(c4ccc(-c5cc6ccccc6c6ccccc56)cc4)c4cccc(-c5cc6ccccc6c6ccccc56)c4)cccc32)cc1 ; c1ccc(-n2c3ccccc3c3cc(N(c4ccc(-c5cc6ccccc6c6ccccc56)cc4)c4ccc5oc6ccccc6c5c4)ccc32)cc1 ; c1ccc(-n2c3ccccc3c3cc(N(c4ccc(-c5cc6ccccc6c6ccccc56)cc4)c4ccc5sc6ccccc6c5c4)ccc32)cc1]
